SEQUENCE LISTING (184 sequences)

SEQ ID NO: 1 - protein, 5 aa, unidentified
  source 1..5: note = Belimumab HCDR1
NNAIN

SEQ ID NO: 2 - protein, 17 aa, unidentified
  source 1..17: note = Belimumab HCDR2
GIIPMFGTAKYSQNFQG

SEQ ID NO: 3 - protein, 14 aa, unidentified
  source 1..14: note = Belimumab HCDR3
SRDLLLFPHHALSP

SEQ ID NO: 4 - protein, 12 aa, unidentified
  source 1..12: note = Belimumab LCDR1
MQGDSLRSYYAS

SEQ ID NO: 5 - protein, 7 aa, unidentified
  source 1..7: note = Belimumab LCDR2
GKNNRPS

SEQ ID NO: 6 - protein, 11 aa, unidentified
  source 1..11: note = Belimumab LCDR3
SSRDSSGNHWV

SEQ ID NO: 7 - protein, 123 aa, unidentified
  source 1..123: note = Belimumab VH
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 8 - protein, 109 aa, unidentified
  source 1..109: note = Belimumab VL
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 9 - protein, 453 aa, unidentified
  source 1..453: note = Belimumab Heavy Chain
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, unidentified
  source 1..214: note = Belimumab Light Chain
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 5 aa, unidentified
  source 1..5: note = Tabalumab HCDR1
GYYWS

SEQ ID NO: 12 - protein, 16 aa, unidentified
  source 1..16: note = Tabalumab HCDR2
EINHSGSTNYNPSLKS

SEQ ID NO: 13 - protein, 15 aa, unidentified
  source 1..15: note = Tabalumab HCDR3
GYYDILTGYYYYFDY

SEQ ID NO: 14 - protein, 11 aa, unidentified
  source 1..11: note = Tabalumab LCDR1
RASQSVSRYLA

SEQ ID NO: 15 - protein, 7 aa, unidentified
  source 1..7: note = Tabalumab LCDR2
DASNRAT

SEQ ID NO: 16 - protein, 9 aa, unidentified
  source 1..9: note = Tabalumab LCDR3
QQRSNWPRT

SEQ ID NO: 17 - protein, 123 aa, unidentified [sequence omitted]

SEQ ID NO: 18 - protein, 107 aa, unidentified [sequence omitted]

SEQ ID NO: 19 - protein, 450 aa, unidentified
  source 1..450: note = Tabalumab Heavy Chain
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, unidentified
  source 1..214: note = Tabalumab Light Chain
EIVLTQSPATLSLSPGERATLSCRASQSVSRYLAWYQQKPGQAPRLLIYDASNRATGIPARFSGSGSGTDSTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 21 - protein, 5 aa, unidentified
  source 1..5: note = rozibafusp alfa HCDR1
SYWMS

SEQ ID NO: 22 - protein, 17 aa, unidentified
YIKQDGNEKYYVDSVKG

SEQ ID NO: 23 - protein, 12 aa, unidentified
  source 1..12: note = rozibafusp alfa HCDR3
EGILWFGDLPTF

SEQ ID NO: 24 - protein, 11 aa, unidentified
  source 1..11: note = rozibafusp alfa LCDR1
RASQGISNWLA

SEQ ID NO: 25 - protein, 7 aa, unidentified
  source 1..7: note = rozibafusp alfa LCDR2
AASSLQS

SEQ ID NO: 26 - protein, 9 aa, unidentified
  source 1..9: note = rozibafusp alfa LCDR3
QQYDSYPRT

SEQ ID NO: 27 - protein, 121 aa, unidentified
  source 1..121: note = rozibafusp alfa anti-ICOSL VH
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSS

SEQ ID NO: 28 - protein, 107 aa, unidentified
  source 1..107: note = rozibafusp alfa anti-ICOSL VL
DIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIK

SEQ ID NO: 29 - protein, 214 aa, unidentified [sequence omitted]

SEQ ID NO: 30 - protein, 448 aa, unidentified
  source 1..448: note = rozibafusp alfa anti-ICOSL Heavy Chain
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKG

SEQ ID NO: 31 - protein, 511 aa, unidentified
  source 1..511: note = rozibafusp alfa bispecific ICOSL Heavy Chain with 2x BAFF binding protein fusion
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGGLPGCKWDLLIKQWVCDPLGSGSATGGSGSVASSGSGSATHLLPGCKWDLLIKQWVCDPL

SEQ ID NO: 32 - protein, 4 aa, unidentified
  source 1..4: note = rozibafusp alfa Heavy Chain Linker
GGGG

SEQ ID NO: 33 - protein, 23 aa, unidentified
  source 1..23: note = rozibafusp alfa BAFF binding protein Linker
GSGSATGGSGSVASSGSGSATHL

SEQ ID NO: 34 - protein, 18 aa, unidentified
  source 1..18: note = rozibafusp alfa BAFF-Binding Protein
LPGCKWDLLIKQWVCDPL

SEQ ID NO: 35 - protein, 290 aa, unidentified
  source 1..290: note = Blisibimod (fusion protein)
GCKWDLLIKQWVCDPLGSGSATGGSGSTASSGSGSATHMLPGCKWDLLIKQWVCDPLGGGGGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 16 aa, unidentified
  source 1..16: note = Blisibimod BAFF binding protein
GCKWDLLIKQWVCDPL

SEQ ID NO: 37 - protein, 18 aa, synthetic construct
  source 1..18: note = Blisibimod BAFF binding protein
LPGCKWDLLIKQWVCDPL

SEQ ID NO: 38 - protein, 23 aa, unidentified
  source 1..23: note = Blisibimod BAFF binding protein Linker
GSGSATGGSGSTASSGSGSATHM

SEQ ID NO: 39 - protein, 6 aa, unidentified
  source 1..6: note = Blisibimod Linker to hIgG1
GGGGGV

SEQ ID NO: 40 - protein, 81 aa, unidentified
  source 1..81: note = TACI ectodomain (blocks BAFF and APRIL binding)
AMRSCPEEQYWDPLLGTCMSCKTICNHQSQRTCAAFCRSLSCRKEQGKFYDHLLRDCISCASICGQHPKQCAYFCENKLRS

SEQ ID NO: 41 - protein, 313 aa, unidentified
  source 1..313: note = Atacicept (TACI ectodomain fused to hIgG1 Fc)
AMRSCPEEQYWDPLLGTCMSCKTICNHQSQRTCAAFCRSLSCRKEQGKFYDHLLRDCISCASICGQHPKQCAYFCENKLRSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 714 aa, unidentified [sequence omitted]

SEQ ID NO: 43 - protein, 119 aa, unidentified
  source 1..119: note = Ixekizumab anti-IL-17 VH from tibulizumab
QVQLVQSGAEVKKPGSSVKVSCKASGYKFTDYHIHWVRQAPGQCLEWMGVINPTYGTTDYNQRFKGRVTITADESTSTAYMELSSLRSEDTAVYYCARYDYFTGTGVYWGQGTLVTVSS

SEQ ID NO: 44 - protein, 112 aa, unidentified
  source 1..112: note = Ixekizumab anti-IL-17 VL from tibulizumab
DIVMTQTPLSLSVTPGQPASISCRSSRSLVHSRGETYLHWYLQKPGQSPQLLIYKVSNRFIGVPDRFSGSGSGTDFTLKISRVEAEDVGVYYCSQSTHLPFTFGCGTKLEIK

SEQ ID NO: 45 - protein, 251 aa, unidentified
  source 1..251: note = Ixekizumab anti-IL-17 scFv
QVQLVQSGAEVKKPGSSVKVSCKASGYKFTDYHIHWVRQAPGQCLEWMGVINPTYGTTDYNQRFKGRVTITADESTSTAYMELSSLRSEDTAVYYCARYDYFTGTGVYWGQGTLVTVSSGGGGSGGGGSGGGGSGGGGSDIVMTQTPLSLSVTPGQPASISCRSSRSLVHSRGETYLHWYLQKPGQSPQLLIYKVSNRFIGVPDRFSGSGSGTDFTLKISRVEAEDVGVYYCSQSTHLPFTFGCGTKLEIK

SEQ ID NO: 46 - protein, 14 aa, synthetic construct
  source 1..14: note = Tibulizumab Heavy Chain Linker
GGGSGGGGTGGGGS

SEQ ID NO: 47 - protein, 20 aa, unidentified
  source 1..20: note = Tibulizumab anti-IL-17 scFv Linker
GGGGSGGGGSGGGGSGGGGS

SEQ ID NO: 48 - protein, 72 aa, unidentified
  source 1..72: note = BAFF-R Ectodomain
DVRRGPRSLRGRDAPAPTPCNPAECFDPLVRHCVACGLLRTPRPKPAGASSPAPRTALQPQESVGAGAGEAA

SEQ ID NO: 49 - protein, 299 aa, unidentified
  source 1..299: note = Briobacept (BAFF-R ectodomain fused to hIgG1 Fc)
DVRRGPRSLRGRDAPAPTPCNPAECFDPLVRHCVACGLLRTPRPKPAGASSPAPRTALQPQESVGAGAGEAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 74 aa, unidentified
  source 1..74: note = Ianalumab
GRIYYRSKWYNSYAVSVKSRITINPDTSKNQFSLQLNSVTPEDTAVYYCARYQWVPKIGVFDSWGQGTLVTVSS

SEQ ID NO: 51 - protein, 108 aa, unidentified
  source 1..108: note = Ianalumab VL
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGQGTKVEIK

SEQ ID NO: 52 - protein, 404 aa, unidentified
  source 1..404: note = Ianalumab Heavy Chain
GRIYYRSKWYNSYAVSVKSRITINPDTSKNQFSLQLNSVTPEDTAVYYCARYQWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 215 aa, unidentified
  source 1..215: note = Ianalumab Light Chain
DIVLTQSPATLSLSPGERATLSCRASQFILPEYLSWYQQKPGQAPRLLIYGSSSRATGVPARFSGSGSGTDFTLTISSLEPEDFAVYYCQQFYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 121 aa, unidentified [sequence omitted]

SEQ ID NO: 55 - protein, 112 aa, unidentified [sequence omitted]

SEQ ID NO: 56 - protein, 451 aa, unidentified
  source 1..451: note = Vedolizumab Heavy Chain
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 219 aa, unidentified
  source 1..219: note = Vedolizumab Light Chain
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 117 aa, unidentified [sequence omitted]

SEQ ID NO: 59 - protein, 107 aa, unidentified [sequence omitted]

SEQ ID NO: 60 - protein, 446 aa, unidentified
  source 1..446: note = Etrolizumab Heavy Chain
EVQLVESGGGLVQPGGSLRLSCAASGFFITNNYWGWVRQAPGKGLEWVGYISYSGSTSYNPSLKSRFTISRDTSKNTFYLQMNSLRAEDTAVYYCARTGSSGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 214 aa, unidentified
  source 1..214: note = Etrolizumab Light Chain
DIQMTQSPSSLSASVGDRVTITCRASESVDDLLHWYQQKPGKAPKLLIKYASQSISGVPSRFSGSGSGTDFTLTISSLQPEDFATYYCQQGNSLPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 121 aa, unidentified
  source 1..121: note = Bleselumab VH
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 63 - protein, 56 aa, unidentified
  source 1..56: note = Bleselumab VL
ASNLESGVPSRFSGSGSGTDFTLTISSLQPEDFATYYCQQFNSYPTFGQGTKVEIK

SEQ ID NO: 64 - protein, 448 aa, unidentified
  source 1..448: note = Bleselumab Heavy Chain
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 212 aa, unidentified
  source 1..212: note = Bleselumab Light Chain
AIQLTQSPSSLSASVGDRVTITCRASQGISSLAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 120 aa, unidentified
  source 1..120: note = Iscalimab (or lucatumumab) VH
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 67 - protein, 112 aa, unidentified
  source 1..112: note = Iscalimab (or lucatumumab) VL
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 68 - protein, 450 aa, unidentified
  source 1..450: note = Iscalimab Heavy Chain
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 219 aa, unidentified
  source 1..219: note = Iscalimab Light Chain
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 116 aa, unidentified
  source 1..116: note = Ravagalimab VH
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSS

SEQ ID NO: 71 - protein, 113 aa, unidentified
  source 1..113: note = Ravagalimab VL
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIK

SEQ ID NO: 72 - protein, 446 aa, unidentified
  source 1..446: note = Ravagalimab Heavy Chain
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 73 - protein, 220 aa, unidentified
  source 1..220: note = Ravagalimab Light Chain
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 114 aa, unidentified [sequence omitted]

SEQ ID NO: 75 - protein, 112 aa, unidentified [sequence omitted]

SEQ ID NO: 76 - protein, 112 aa, unidentified
  source 1..112: note = Giloralimab VH
EVQLQESGPGLVKPSETLSLTCTVSGYSITSNYYWNWIRQPPGKGLEWMGYIRYDGSNNYNPSLKNRVTISRDTSKNQFSLKLSSVTAADTAVYYCARLDYWGQGTTVTVSS

SEQ ID NO: 77 - protein, 112 aa, unidentified
  source 1..112: note = Giloralimab VL
DIVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 78 - protein, 119 aa, unidentified
  source 1..119: note = Mitazalimab VH
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 79 - protein, 111 aa, unidentified
  source 1..111: note = Mitazalimab VL
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVL

SEQ ID NO: 80 - protein, 126 aa, unidentified
  source 1..126: note = Selicrelumab VH
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 81 - protein, 107 aa, unidentified
  source 1..107: note = Selicrelumab VL
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 82 - protein, 120 aa, unidentified
  source 1..120: note = Sotigalimab VH
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 83 - protein, 108 aa, unidentified
  source 1..108: note = Sotigalimab VL
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 84 - protein, 119 aa, unidentified; identical to SEQ ID NO: 78
  source 1..119: note = Vanalimab VH

SEQ ID NO: 85 - protein, 111 aa, unidentified; identical to SEQ ID NO: 79
  source 1..111: note = Vanalimab VL

SEQ ID NO: 86 - protein, 118 aa, unidentified
  source 1..118: note = dapirolizumab pegol VH
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSS

SEQ ID NO: 87 - protein, 107 aa, unidentified
  source 1..107: note = dapirolizumab pegol VL
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIK

SEQ ID NO: 88 - protein, 229 aa, unidentified
  source 1..229: note = dapirolizumab pegol Heavy Chain
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 89 - protein, 214 aa, unidentified
  source 1..214: note = dapirolizumab pegol Light Chain
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 118 aa, unidentified
  source 1..118: note = letolizumab VH
EVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSS

SEQ ID NO: 91 - protein, 353 aa, unidentified
  source 1..353: note = letolizumab (anti-CD40L IgG1 Fc fusion protein)
EVQLLESGGGLVQPGGSLRLSCAASGFTFNWELMGWARQAPGKGLEWVSGIEGPGDVTYYADSVKGRFTISRDNSKNTLYLQMNSLRAEDTAVYYCVKVGKDAKSDYRGQGTLVTVSSASTEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 785 aa, unidentified
  source 1..785: note = Dazodalibep (anti-CD40L-HSA fusion protein)
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 93 - protein, 85 aa, unidentified
  source 1..85: note = anti-CD40L var1
SQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 94 - protein, 90 aa, unidentified
  source 1..90: note = anti-CD40L var2
RLDAPSQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 95 - protein, 585 aa, unidentified
  source 1..585: note = human serum albumin
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 96 - protein, 15 aa, unidentified
  source 1..15: note = anti-CD40L Gly linker
GGGGGGGGGGGGGGG

SEQ ID NO: 97 - protein, 10 aa, unidentified
  source 1..10: note = albumin linker
GGGGGGGGGG

SEQ ID NO: 98 - protein, 125 aa, unidentified
  source 1..125: note = CTLA4 Ectodomain (high affinity variant from belatacept)
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQ

SEQ ID NO: 99 - protein, 357 aa, unidentified
  source 1..357: note = Belatacept fusion protein
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 125 aa, unidentified
  source 1..125: note = CTLA4 Ectodomain from abatacept
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQ

SEQ ID NO: 101 - protein, 357 aa, unidentified [sequence omitted]

SEQ ID NO: 102 - protein, 125 aa, unidentified
  source 1..125: note = CTLA4 Ectodomain Asp2408 variant from abatacept
MHVAQPAVVLASSRGIASFVCEYASPGKANEVRVTVLRQADSQVTEVCAMTYMKENELTFLDDPICTGTFSGNQVNLTIQGLRAMDTGLYICKVELMYPPPYYLGIGNGTQIYVIDPEPCPDSDQ

SEQ ID NO: 103 - protein, 357 aa, unidentified
  source 1..357: note = Abatacept variant 1 CTLA4-hIgG1 Fc (CTLA4 Asp2408 variant)
MHVAQPAVVLASSRGIASFVCEYASPGKANEVRVTVLRQADSQVTEVCAMTYMKENELTFLDDPICTGTFSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 125 aa, unidentified
  source 1..125: note = CTLA4 Ectodomain Asp2409 variant from abatacept
MHVAQPAVVLASSRGIASFVCEYESPGKANEIRVTVLRQADSQVTEVCAMTYMKGDELTFLDDPSCTGTFSGNQVNLTIQGLRAMDTGLYICKVELMYPPPYYEGIGNGTQIYVIDPEPCPDSDQ

SEQ ID NO: 105 - protein, 357 aa, unidentified
  source 1..357: note = Abatacept variant 2 CTLA4-hIgG1 Fc (CTLA4 Asp2409 variant)
MHVAQPAVVLASSRGIASFVCEYESPGKANEIRVTVLRQADSQVTEVCAMTYMKGDELTFLDDPSCTGTFSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 124 aa, unidentified
  source 1..124: note = Alomfilimab VH
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 107 - protein, 108 aa, unidentified
  source 1..108: note = Alomfilimab VL
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 108 - protein, 121 aa, unidentified
  source 1..121: note = Feladilimab VH
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 109 - protein, 106 aa, unidentified
  source 1..106: note = Feladilimab VL
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 110 - protein, 125 aa, unidentified
  source 1..125: note = Izuralimab (Bispecific mAb scFv targeting ICOS and PD-1 ICOS binding domain provided) VH
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYYCARTYYYDSSGYYHDAFDIWGQGTMVTVSS

SEQ ID NO: 111 - protein, 107 aa, unidentified
  source 1..107: note = Feladilimab VL
DIQMTQSPSSVSASVGDRVTITCRASQGISRLLAWYQQKPGKAPKLLIYVASSLQSGVPSRFSGSGSGTDFTLTISSLQPEDFATYYCQQANSFPWTFGQGTKVEIK

SEQ ID NO: 112 - protein, 117 aa, unidentified
  source 1..117: note = Vopratelimab VH
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 113 - protein, 111 aa, unidentified
  source 1..111: note = Vopratelimab VL
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 114 - protein, 122 aa, unidentified
  source 1..122: note = ICOSL N-terminal fragment (used in acazicolcept)
DTQEKEVRAMVGSDVELSCACPEGSRFDLNDVYVYWQTSESKTVVTYHIPQHSSLEYVDSRYRNRALMSPAGMLRGDFSLRLFNVTPQDEQKFHCLVLSRSLGFQEVLSVEVTLHVAANFSV

SEQ ID NO: 115 - protein, 363 aa, unidentified
  source 1..363: note = acazicolcept (ICOSL fragment-null huFc fusion protein)
DTQEKEVRAMVGSDVELSCACPEGSRFDLNDVYVYWQTSESKTVVTYHIPQHSSLEYVDSRYRNRALMSPAGMLRGDFSLRLFNVTPQDEQKFHCLVLSRSLGFQEVLSVEVTLHVAANFSVGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 10 aa, unidentified
  source 1..10: note = ICOSL Gly-Ser linker
GGGGSGGGGS

SEQ ID NO: 117 - protein, 108 aa, unidentified
  source 1..108: note = Lulizumab pegol (pegylated anti-CD28 V-kappa domain antibody (dAb))
DIQMTQSPSSLSASVGDRVTITCRASRPIWPFLEWYQQKPGKAPKLLIYFTSRLRHGVPSRFSGSGSGTCFTLTISSLQPEDFATYYCLQNVANPATFSQGTKVEIKR

SEQ ID NO: 118 - protein, 118 aa, unidentified [sequence omitted]

SEQ ID NO: 119 - protein, 107 aa, unidentified [sequence omitted]

SEQ ID NO: 120 - protein, 123 aa, unidentified [sequence omitted]

SEQ ID NO: 121 - protein, 106 aa, unidentified [sequence omitted]

SEQ ID NO: 122 - protein, 222 aa, unidentified
  source 1..222: note = Natalizumab Heavy Chain
QVQLVQSGAEVKKPGASVKVSCKASGFNIKDTYIHWVRQAPGQRLEWMGRIDPANGYTKYDPKFQGRVTITADTSASTAYMELSSLRSEDEAVYYCAREGYYGNYGVYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVE

SEQ ID NO: 123 - protein, 210 aa, unidentified
  source 1..210: note = Natalizumab Light Chain
DIQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 124 - protein, 124 aa, unidentified
  source 1..124: note = Ontamalimab VH
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSS

SEQ ID NO: 125 - protein, 112 aa, unidentified
  source 1..112: note = Ontamalimab VL
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIK

SEQ ID NO: 126 - protein, 450 aa, unidentified
  source 1..450: note = Ontamalimab Heavy Chain
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 127 - protein, 219 aa, unidentified
  source 1..219: note = Ontamalimab Light Chain
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 5 aa, unidentified
  source 1..5: note = adalimumab HCDR1
DYAMH

SEQ ID NO: 129 - protein, 17 aa, unidentified
  source 1..17: note = adalimumab HCDR2
AITWNSGHIDYADSVEG

SEQ ID NO: 130 - protein, 12 aa, unidentified
  source 1..12: note = adalimumab HCDR3
VSYLSTASSLDY

SEQ ID NO: 131 - protein, 11 aa, unidentified
  source 1..11: note = adalimumab LCDR1
RASQGIRNYLA

SEQ ID NO: 132 - protein, 7 aa, unidentified
  source 1..7: note = adalimumab LCDR2
AASTLQS

SEQ ID NO: 133 - protein, 9 aa, unidentified
  source 1..9: note = adalimumab LCDR3
QRYNRAPYT

SEQ ID NO: 134 - protein, 121 aa, unidentified [sequence omitted]

SEQ ID NO: 135 - protein, 107 aa, unidentified [sequence omitted]

SEQ ID NO: 136 - protein, 54 aa, unidentified
  source 1..54: note = Etanercept TNFR-CYS Repeat 1
LPAQVAFTPYAPEPGSTCRLREYYDQTAQMCCSKCSPGQHAKVFCTKTSDTVCD

SEQ ID NO: 137 - protein, 42 aa, unidentified
  source 1..42: note = Etanercept TNFR-CYS Repeat 2
SCEDSTYTQLWNWVPECLSCGSRCSSDQVETQACTREQNRIC

SEQ ID NO: 138 - protein, 44 aa, unidentified
  source 1..44: note = Etanercept TNFR-CYS Repeat 3
TCRPGWYCALSKQEGCRLCAPLRKCRPGFGVARPGTETSDVVCK

SEQ ID NO: 139 - protein, 39 aa, unidentified
  source 1..39: note = Etanercept TNFR-CYS Repeat 4
PCAPGTFSNTTSSTDICRPHQICNVVAIPGNASMDAVCT

SEQ ID NO: 140 - protein, 56 aa, unidentified
  source 1..56: note = Etanercept TNFR-CYS Repeat 5
STSPTRSMAPGAVHLPQPVSTRSQHTQPTPEPSTAPSTSFLLPMGPSPPAEGSTGD

SEQ ID NO: 141 - protein, 467 aa, unidentified
  source 1..467: note = Etanercept fusion protein
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 5 aa, unidentified
  source 1..5: note = golimumab HCDR1
SYAMH

SEQ ID NO: 143 - protein, 17 aa, unidentified
  source 1..17: note = golimumab HCDR2
FMSYDGSNKKYADSVKG

SEQ ID NO: 144 - protein, 17 aa, unidentified
  source 1..17: note = golimumab HCDR3
DRGIAAGGNYYYYGMDV

SEQ ID NO: 145 - protein, 11 aa, unidentified
  source 1..11: note = golimumab LCDR1
RASQSVYSYLA

SEQ ID NO: 146 - protein, 7 aa, unidentified
  source 1..7: note = golimumab LCDR2
DASNRAT

SEQ ID NO: 147 - protein, 10 aa, unidentified
  source 1..10: note = golimumab LCDR3
QQRSNWPPFT

SEQ ID NO: 148 - protein, 126 aa, unidentified
  source 1..126: note = golimumab VH
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 149 - protein, 111 aa, unidentified
  source 1..111: note = golimumab VL
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTV

SEQ ID NO: 150 - protein, 456 aa, unidentified
  source 1..456: note = golimumab Heavy Chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 215 aa, unidentified
  source 1..215: note = golimumab Light Chain Heavy Chain
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 119 aa, unidentified
  source 1..119: note = infliximab VH Heavy Chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 153 - protein, 107 aa, unidentified
  source 1..107: note = infliximab VL Heavy Chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 154 - protein, 5 aa, unidentified
  source 1..5: note = infliximab HCDR1 Heavy Chain
NHWMN

SEQ ID NO: 155 - protein, 19 aa, unidentified
  source 1..19: note = infliximab HCDR2 Heavy Chain
EIRSKSINSATHYAESVKG

SEQ ID NO: 156 - protein, 9 aa, unidentified
  source 1..9: note = infliximab HCDR3 Heavy Chain
NYYGSTYDY

SEQ ID NO: 157 - protein, 11 aa, unidentified
  source 1..11: note = infliximab LCDR1 Heavy Chain
RASQFVGSSIH

SEQ ID NO: 158 - protein, 7 aa, unidentified
  source 1..7: note = infliximab LCDR2 Heavy Chain
YASESMS

SEQ ID NO: 159 - protein, 9 aa, unidentified
  source 1..9: note = infliximab LCDR3 Heavy Chain
QQSHSWPFT

SEQ ID NO: 160 - protein, 5 aa, unidentified
  source 1..5: note = certolizumab pegol HCDR1 Heavy Chain
DYGMN

SEQ ID NO: 161 - protein, 17 aa, unidentified
  source 1..17: note = certolizumab pegol HCDR2
WINTYIGEPIYADSVKG

SEQ ID NO: 162 - protein, 9 aa, unidentified
  source 1..9: note = certolizumab pegol HCDR3
GYRSYAMDY

SEQ ID NO: 163 - protein, 7 aa, unidentified
  source 1..7: note = certolizumab pegol LCDR1
NVGTNVA

SEQ ID NO: 164 - protein, 7 aa, unidentified
  source 1..7: note = certolizumab pegol LCDR2
SASFLYS

SEQ ID NO: 165 - protein, 10 aa, unidentified
  source 1..10: note = certolizumab pegol LCDR3
QQYNIYPLTF

SEQ ID NO: 166 - protein, 229 aa, unidentified
  source 1..229: note = certolizumab pegol VH
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 167 - protein, 214 aa, unidentified
  source 1..214: note = certolizumab pegol VL
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 118 aa, unidentified
  source 1..118: note = certolizumab pegol VH
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 169 - protein, 120 aa, unidentified
  source 1..120: note = Sibeprenlimab VH
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSS

SEQ ID NO: 170 - protein, 111 aa, unidentified
  source 1..111: note = Sibeprenlimab VL
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIK

SEQ ID NO: 171 - protein, 446 aa, unidentified
  source 1..446: note = Sibeprenlimab Heavy Chain
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 172 - protein, 218 aa, unidentified
  source 1..218: note = Sibeprenlimab Light Chain
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 8 aa, unidentified
  source 1..8: note = BION-1301 HCDR1
GYTFTSYV

SEQ ID NO: 174 - protein, 16 aa, unidentified
  source 1..16: note = BION-1301 HCDR2
YINPYNDAPKYNEKFK

SEQ ID NO: 175 - protein, 12 aa, unidentified
  source 1..12: note = BION-1301 HCDR3
GLGYALYYAMDY

SEQ ID NO: 176 - protein, 11 aa, unidentified
  source 1..11: note = BION-1301 LCDR1
KASQNVGNNVA

SEQ ID NO: 177 - protein, 7 aa, unidentified
  source 1..7: note = BION-1301 LCDR2
SASNRDS

SEQ ID NO: 178 - protein, 10 aa, unidentified
  source 1..10: note = BION-1301 LCDR3
QQYNIYPFTF

SEQ ID NO: 179 - protein, 121 aa, unidentified
  source 1..121: note = BION-1301 VH
QVQLVQSGAEVKKPGASVKVSCKASGYTFTSYVMHWVRQAPGQGLEWMGYINPYNDAPKYNEKFKGRVTMTRDTSISTAYMELSRLRSDDTAVYYCARGLGYALYYAMDYWGQGTTVTVSS

SEQ ID NO: 180 - protein, 107 aa, unidentified
  source 1..107: note = BION-1301 VL
DIVMTQSPSTLSASVGDRVTITCKASQNVGNNVAWYQQKPGKAPKLLISSASNRDSGVPSRFSGSGSGTEFTLTISSLQPDDFATYFCQQYNIYPFTFGQGTKLEIK

SEQ ID NO: 181 - protein, 330 aa, unidentified
  source 1..330: note = human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 330 aa, unidentified
  source 1..330: note = human IgG1 null constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 107 aa, unidentified [sequence omitted]

SEQ ID NO: 184 - protein, 449 aa, unidentified
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

(19) United States
(12) Patent Application Publication
(10) Pub. No.: US 2024/0218011 A1
(43) Pub. Date: Jul. 4, 2024

(54) GLUCOCORTICOID RECEPTOR AGONISTS AND CONJUGATES THEREOF

(71) Applicant: FireFly Bio, Inc., South San Francisco, CA (US)

(72) Inventors: Sean Wesley Smith, Seattle, WA (US); Scott Allan Mitchell, Seattle, WA (US); Jack Chang Hung Lee, Seattle, WA (US)

(21) Appl. No.: 18/356,148

(22) Filed: Jul. 20, 2023

Related U.S. Application Data
(60) Provisional application No. 63/368,989, filed on Jul. 21, 2022.
(60) Provisional application No. 63/489,700, filed on Mar. 10, 2023.

(51) Int. Cl. C07J 43/00; A61K 31/58; A61K 47/64; C07J 9/00; C07J 17/00; C07J 33/00

(52) U.S. Cl. CPC C07J 43/003; A61K 31/58; A61K 47/6425; C07J 9/00; C07J 17/00; C07J 33/002

(57) ABSTRACT

The present disclosure describes glucocorticoid agonist compounds, their conjugates with binding proteins, pharmaceutical compositions thereof, as well as methods and uses for treating diseases or conditions, such as autoimmune or inflammatory conditions.

### BACKGROUND

Autoimmune and inflammatory diseases are immune cell driven chronic conditions that exact a high toll on quality of life for patients often resulting in a shortened lifespan through disease mediated organ damage. Glucocorticoid receptor agonists (GRA) and modulators (GRM) have been a mainstay for controlling many autoimmune and inflammatory diseases by decreasing immune cell disease activities. However, given the wide cell type and tissue expression of the glucocorticoid receptor, as well as the potent activity of the axis on many cellular functions, systemic administration of GRA or GRM results in a diverse set of unacceptable toxicities in both a dose- and duration-dependent manner (such as deleterious bone, cardiovascular, metabolic, and neuropsychiatric toxicities, among others), even at low doses that are necessary to afford some disease relief.

Medicinal chemistry attempts to direct effective agonism into disease driving immune cells without toxicity-generating agonism in other cell types s by using systemically delivered small molecule GRA or GRM designed to activate a subset of glucocorticoid receptor (GR) cellular activities, have to date been disappointing, either due to inadequate disease control, still unacceptable toxicities, or both. Given this, current practicing guidelines for many autoimmune and inflammatory disease are to limit both dose and duration of GRA or GRM treatment despite evidence for decreased loss of disease control when this is done. Therefore, there is a need for alternative strategies for therapies to treat autoimmune or inflammatory conditions.

### BRIEF SUMMARY

In some embodiments, the present invention provides a compound of Formula II:

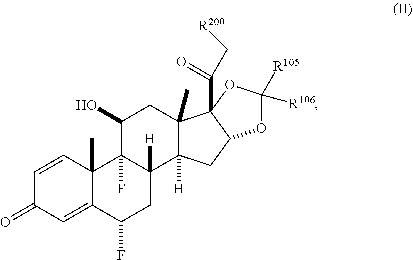

or a pharmaceutically acceptable salt thereof, wherein R¹⁰⁵, R¹⁰⁶, and R²⁰⁰ are defined herein, as well as conjugates, pharmaceutical compositions, methods, and uses thereof.

### DETAILED DESCRIPTION

### I. General

The present disclosure describes glucocorticoid agonist compounds, their conjugates with binding proteins, pharmaceutical compositions thereof, as well as methods and uses for treating diseases or conditions, such as autoimmune or inflammatory conditions.

### II. Definitions

“Alkyl” is a linear or branched saturated monovalent hydrocarbon. For example, an alkyl group can have 1 to 18 carbon atoms (i.e., C₁₋₁₈ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH3)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include heptyl, octyl, nonyl, decyl, undecyl, dodecyl, pentadcyl, hexadecyl, heptadecyl and octadecyl.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆.

Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers an alkoxy group linked to an alkyl group which is linked to the remainder of the compound such that the alkyl group is divalent. Alkoxyalkyl can have any suitable number of carbons, such as from 2 to 6 (C₂₋₆ alkoxyalkyl), 2 to 5 (C₂₋₅ alkoxyalkyl), 2 to 4 (Ca-4 alkoxyalkyl), or 2 to 3 (C₂₋₃ alkoxyalkyl). The number of carbons refers to the total number of carbons in the alkoxy and the alkyl group. For example, C₆ alkoxyalkyl refers to ethoxy (C₂ alkoxy) linked to a butyl (C₄ alkyl), and n-propoxy (C₃ alkoxy) linked to a isopropyl (C₃ alkyl). Alkoxy and alkyl are as defined above where the alkyl is divalent, and can include, but is not limited to, methoxymethyl (CH₃OCH₂—), methoxyethyl (CH₃OCH₂CH₂—) and others.

“Halo” or “halogen” as used herein refers to fluoro (—F), chloro (—Cl), bromo (—Br) and iodo (—I).

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated, and partially unsaturated all carbon ring systems (e.g., ring systems comprising 2, 3 or 4 carbocyclic rings). Accordingly, cycloalkyl includes multicyclic carbocyles such as a bicyclic carbocycles (e.g., bicyclic carbocycles having 6 to 12 annular carbon atoms such as bicyclo[3.1.0]hexane and bicyclo[2.1.1]hexane), and polycyclic carbocycles (e.g., tricyclic and tetracyclic carbocycles with up to 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Heterocyclyl” or “heterocycle” or “heterocycloalkyl” as used herein refers to a single saturated or partially unsaturated non-aromatic ring or a multiple ring system having at least one heteroatom in the ring (i.e., at least one annular heteroatom selected from oxygen, nitrogen, and sulfur) wherein the multiple ring system includes at least non-aromatic ring containing at least one heteroatom. The multiple ring system can also include other aromatic rings and non-aromatic rings. Unless otherwise specified, a heterocyclyl group has from 3 to 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from 1 to 6 annular carbon atoms and from 1 to 3 annular heteroatoms selected from the group consisting of oxygen, nitrogen, and sulfur in the ring. The heteroatoms can optionally be oxidized to form —N(—OH)—, ═N(—O⁻)—, —S(═O)— or —S(═O)₂—. The rings of the multiple condensed ring (e.g., bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like.

Heterocycloalkyl rings also include 9 to 15 membered fused ring heterocycloalkyls having 2, 3, or more rings wherein at least one ring is an aryl ring and at least one ring is a non-aromatic ring containing at least one heteroatom. Representative fused bicyclic heterocycloalkyls include, but are not limited to, indoline (dihydroindole), isoindoline (dihydroisoindole), indazoline (dihydroindazole), benzo[d]imidazole, dihydroquinoline, dihydroisoquinoline, dihydrobenzofuran, dihydroisobenzofuran, benzo[d][1,3]dioxol, dihydrobenzo[b]dioxine, dihydrobenzo[d]oxazole, dihydrobenzo[b]thiophene, dihydroisobenzo[c]thiophene, dihydrobenzo[d]thiazole, dihydrobenzo[c]isothiazole, and benzo[b][1,4]thiazine, as shown in the structures below:

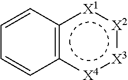

Fused bicyclic heterocycloalkyls can also be represented by the following structure:

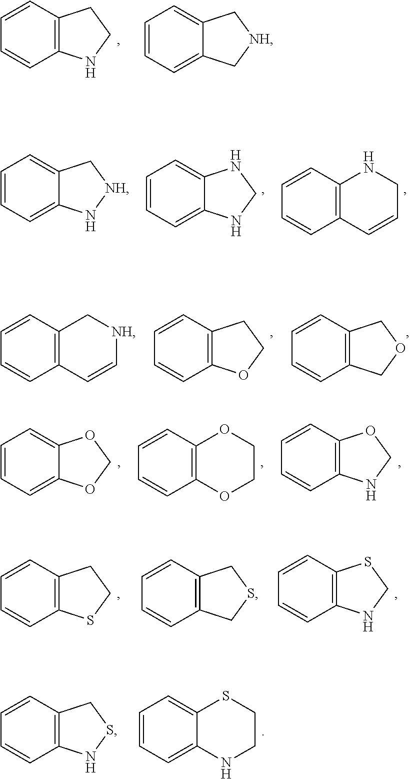

wherein X¹, X², X³ and X⁴ are each independently absent, —CH₂—, —NH—, —O— or —S—, at least one of X¹, X², X³ and X⁴ is —NH—, —O— or —S—, and the dashed circle represents a saturated or partially unsaturated non-aromatic ring. The fused bicyclic heterocycloalkyls are optionally substituted.

“Aryl” as used herein refers to a single all carbon aromatic ring or a multiple condensed all carbon ring system wherein at least one of the rings is aromatic. For example, in some embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having 9 to 20 carbon atoms, e.g., 9 to 16 carbon atoms, in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

“Alkylene-aryl” refers to a radical having an alkylene component and an aryl component, where the alkylene component links the aryl component to the point of attachment. The alkylene component is as defined above to link to the aryl component and to the point of attachment. The alkylene component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The aryl component is as defined above. Examples of alkylene-aryl groups include, but are not limited to, benzyl and ethyl-benzene. Alkylene-aryl groups can be substituted or unsubstituted.

“Heteroaryl” as used herein refers to a single aromatic ring that has at least one atom other than carbon in the ring, wherein the atom is selected from the group consisting of oxygen, nitrogen, and sulfur; “heteroaryl” also includes multiple condensed ring systems that have at least one such aromatic ring, which multiple condensed ring systems are further described below. Thus, “heteroaryl” includes single aromatic rings of from 1 to 6 carbon atoms and 1-4 heteroatoms selected from the group consisting of oxygen, nitrogen, and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has 1-20 carbon atoms and 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, and triazolyl.

“Alkylene-heteroaryl” refers to a radical having an alkylene component and a heteroaryl component, where the alkylene component links the heteroaryl component to the point of attachment. The alkylene component is as defined above to link to the heteroaryl component and to the point of attachment. The alkylene component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined within. Alkylene-heteroaryl groups can be substituted or unsubstituted.

Chemical entities having carbon-carbon double bonds or carbon-nitrogen double bonds may exist in Z- or E- form (or cis- or trans-form). Furthermore, some chemical entities may exist in various tautomeric forms. Unless otherwise specified, compounds of the disclosure are intended to include all Z-, E- and tautomeric forms as well.

A “tautomer” refers to a molecule wherein a proton shift from one atom of a molecule to another atom of the same molecule is possible. The compounds presented herein, in some embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

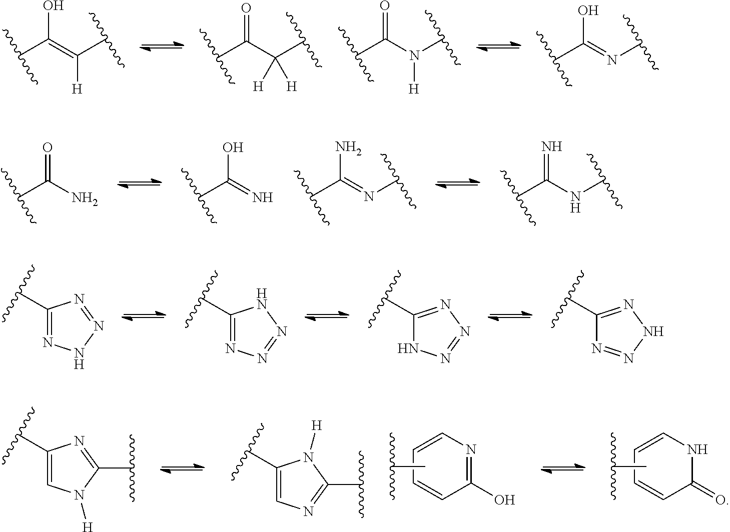

A “compound of the present disclosure” includes compounds disclosed herein, for example a compound of the present disclosure includes compounds of Formula I and II, including the compounds of the Examples.

The compounds of the disclosure may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

“Composition” as used herein is intended to encompass a product comprising the specified ingredients in the specified amounts, as well as any product, which results, directly or indirectly, from combination of the specified ingredients in the specified amounts.

As used herein, the phrase “pharmaceutically acceptable” refers to those compounds, materials, compositions, or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” means a pharmaceutically acceptable material, composition, or vehicle, such as a liquid or solid filler, diluent, excipient, solvent, or encapsulating material. “Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is chosen from ammonium, potassium, sodium, calcium, and magnesium salts.

As used herein, a “binding protein” comprises a polypeptide and a binding domain or an antigen binding fragment thereof that specifically binds to a target or multiple targets. Exemplary binding proteins of this disclosure include fusion proteins, antibodies (e.g., monoclonal antibodies, bispecific antibodies), antibody constructs, targeting moieties, or an antigen binding fragment thereof. In some embodiments, a binding protein of the present disclosure comprises a binding domain of an antibody or an antigen binding fragment thereof. In some embodiments, a binding protein or binding polypeptide of this disclosure (including those in which the term “polypeptide” may be synonymous with the term “protein”) comprises two or more polypeptides. In some embodiments, a binding protein or polypeptide of this disclosure comprises a complex of two or more polypeptides. In some embodiments, a binding protein or polypeptide further comprises a tag, a label, a bioactive molecule, or any combination thereof. In some embodiments, a binding protein or polypeptide of this disclosure comprises a non-naturally occurring amino acid.

In some embodiments, a binding protein or binding polypeptide of this disclosure comprises or consists of a fragment. As used herein, a “polypeptide fragment” means a polypeptide that is lacking one or more amino acid(s) present in a reference sequence, which may be referred to as an “oligopeptide fragment” or “peptide fragment.” In certain embodiments, a polypeptide, oligopeptide, or peptide fragment of this disclosure comprises a deletion of one or more amino acids present in reference polypeptide. In further embodiments, a polypeptide, oligopeptide, or peptide fragment of this disclosure comprises a truncation of one or more amino acids present in reference polypeptide. A polypeptide, oligopeptide, or peptide fragment of this disclosure can comprise a binding domain, an antigen, or an epitope, such as a binding domain, an antigen, or an epitope present in a reference sequence as disclosed herein. A polypeptide fragment of this disclosure may have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of the number of total amino acids of the amino acid sequence of the reference sequence.

“Treatment,” “treat,” or “treating” refer to an intervention that leads to any observable beneficial effect of the treatment or any indicia of statistically significant success in the treatment or amelioration of the disease or condition, such as ameliorating a sign, symptom, or progression of a disease or pathological condition. The beneficial effect can be evidenced by, for example, a reduction, delayed onset, or alleviation of the severity of clinical symptoms of the disease in a subject, a reduction in the frequency with which symptoms of a disease are experienced by a subject, a slower progression of the disease, a reduction in the number of relapses of the disease, an improvement in the overall health or well-being of the subject, or by other parameters that are specific to the particular disease.

A prophylactic treatment meant to “prevent” a disease or condition (e.g., tumor formation or growth, in a subject or patient) is a treatment administered to a subject who does not exhibit signs of a disease or exhibits only early signs, for the purpose of decreasing the risk of developing pathology or further advancement of the early disease. For example, if an individual at risk of developing or having severe symptoms of an inflammatory or autoimmune condition is treated with the methods of the present disclosure and does not later develop or have severe symptoms of an inflammatory or autoimmune condition, then the disease or severity of the disease has been prevented, at least over a period of time, in that individual. A prophylactic treatment can mean preventing recurrence of a disease or condition in a patient that has previously been treated for the disease or condition, e.g., by preventing relapse or recurrence of inflammatory or autoimmune disease.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. The phrases “intravenous administration” and “administered intravenously” as used herein refer to injection or infusion of a conjugate into a vein of a subject. The phrases “subcutaneous administration,” “subcutaneously administering,” or the like refer to administration of a conjugate into the subcutis of a subject. For clarity, a subcutaneous administration is distinct from an intratumoral injection into a tumor or cancerous lesion located in the subcuta.

“GR” refers to the glucocorticoid receptor.

A “GR agonist” is a compound that binds to and activates the glucocorticoid receptor.

### III. Compounds

In some embodiments, the compound of the present disclosure is a compound of Formula I:

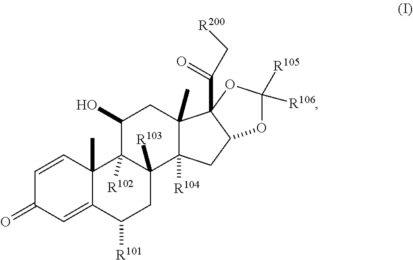

or a pharmaceutically acceptable salt thereof,
wherein
R¹⁰¹, R¹⁰², R¹⁰³, and R¹⁰⁴ are each independently H or F;
R¹⁰⁵ is C₁₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl or heterocyclyl is substituted with 0, 1, 2, or 3 R¹¹⁰;
each R¹⁰⁷ is independently C₂₋₆ alkoxyalkyl, C₁₋₆ haloalkoxy, phenyl, heteroaryl, C₃₋₈ cycloalkyl, heterocyclyl, or halogen, wherein the phenyl is substituted with 0, 1, 2, or 3 R¹¹¹, and the heteroaryl, cycloalkyl, or heterocyclyl is substituted with 0, 1, 2, or 3 R¹¹¹;
each R¹⁰⁸ is independently C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹²;
each R¹⁰⁹ is independently C₁₋₆ alkyl, C₂₋₆ alkenyl, C₂₋₆ alkynyl, C₂₋₆ alkoxyalkyl, C₁₋₆ haloalkyl, halogen, —N₃, —OR¹¹³, or —N(R¹¹³)₂, wherein the alkyl, alkenyl, or alkynyl is substituted with 0 or 1 —S(O)₂(C₁₋₆ alkyl);
each R¹¹⁰ is independently C₂₋₆ alkyl, C₂₋₆ alkenyl, C₂₋₆ alkynyl, C₂₋₆ alkoxyalkyl, —(C₁₋₆ alkylene)-OR¹¹⁴, —(C₁₋₆ alkylene)-N(R¹¹⁴)₂, C₁₋₆ haloalkyl, phenyl, —(C₁₋₆ alkylene)-phenyl, heteroaryl, —(C₁₋₆ alkylene)-heteroaryl, halogen, —N₃, —OR¹¹⁵, —N(R¹¹⁵)₂, —N(R¹¹⁵)(CO)R¹¹⁵, —N(R¹¹⁵)(CO)OR¹¹⁵, —N(R¹¹⁵)S(O)₂R¹¹⁵, —(CO)R¹¹⁵, —SO₂R¹¹⁵, or —SO₂N(R¹¹⁵)₂, wherein the phenyl, alkylene-phenyl, heteroaryl, or alkylene-heteroaryl is substituted with 0, 1, 2, or 3 R¹¹⁶;
each R¹¹¹ and R¹¹² is independently C₁₋₆ alkyl, C₁₋₆ alkoxy, C₁₋₆ haloalkyl, C₁₋₆ haloalkoxy, halogen, —OR¹¹⁴, or —N(R¹¹⁴)₂;
each R¹¹³ is independently H, C₁₋₆ alkyl, C₁₋₆ alkoxy, C₁₋₆ haloalkyl, or phenyl, wherein the haloalkyl is substituted with 0 or 1 N(R¹¹⁴)₂;
each R¹¹⁴ is independently H or C₁₋₆ alkyl;
each R¹¹⁶ is —OR¹¹⁷, —N(R¹¹⁷)₂, —N(R¹¹⁷)(CO)R¹¹⁷, —N(R¹¹⁷)(CO)OR¹¹⁷, —N(R¹¹⁷)S(O)₂R¹¹⁷, —(CO)R¹¹⁷, —SO₂R¹¹⁷, or —SO₂N(R¹¹⁷)₂;
each R¹¹⁵ and R¹¹⁷ is independently H, C₁₋₆ alkyl, C₁₋₆ haloalkyl, or phenyl;
R²⁰⁰ is —OR²⁰¹ or —N(R²⁰¹)₂;
R²⁰¹ is H, C₁₋₆ alkyl, phenyl, or heteroaryl, wherein the phenyl or heteroaryl is substituted with 0, 1, or 2 —OR²⁰² or —N(R²⁰²)₂.
R²⁰² is H or C₁₋₆ alkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, the compound of the present disclosure is a compound of Formula I:

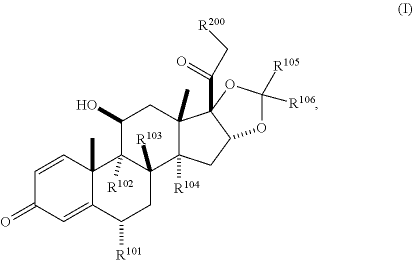

or a pharmaceutically acceptable salt thereof,
wherein
R¹⁰¹, R¹⁰², R¹⁰3, and R¹⁰⁴ are each independently H or F;
R¹⁰⁵ is C₁₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃-s cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl or heterocyclyl is substituted with 0, 1, 2, or 3 R¹¹⁰;
each R¹⁰⁷ is independently C₂₋₆ alkoxyalkyl, C₁₋₆ haloalkoxy, phenyl, heteroaryl, C₃₋₈ cycloalkyl, heterocyclyl, or halogen, wherein the phenyl is substituted with 0, 1, 2, or 3 R¹¹¹, and the heteroaryl, cycloalkyl, or heterocyclyl is substituted with 0, 1, 2, or 3 R¹¹¹;
each R¹⁰⁸ is independently C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹²;
each R¹⁰⁹ is independently C₁₋₆ alkyl, C₂₋₆ alkenyl, C₂₋₆ alkynyl, C₂₋₆ alkoxyalkyl, C₁₋₆ haloalkyl, halogen, —N₃, —OR¹¹³, or —N(R¹¹³)₂, wherein the alkyl, alkenyl, or alkynyl is substituted with 0 or 1 —S(O)₂(C₁₋₆ alkyl);
each R¹¹⁰ is independently C₂₋₆ alkyl, C₂₋₆ alkenyl, C₂₋₆ alkynyl, C₂₋₆ alkoxyalkyl, —(C₁₋₆ alkylene)-OR¹¹⁴, —(C₁₋₆ alkylene)-N(R¹¹⁴)₂, C₁₋₆ haloalkyl, phenyl, —(C₁₋₆ alkylene)-phenyl, heteroaryl, —(C₁₋₆ alkylene)-heteroaryl, halogen, —N3, —OR¹¹⁵, —N(R¹¹⁵)₂, —N(R¹¹¹⁵)(CO)R¹¹⁵, —N(R¹¹¹⁵)(CO)OR¹¹⁵, —N(R¹¹⁵)S(O)₂R¹¹⁵, —(CO)R¹¹⁵, —SO₂R¹¹⁵, or —SO₂N(R¹¹⁵)₂, wherein the phenyl, alkylene-phenyl, heteroaryl, or alkylene-heteroaryl is substituted with 0, 1, 2, or 3 R¹¹⁶;
each R¹¹¹ and R¹¹² is independently C₁₋₆ alkyl, C₁₋₆ alkoxy, C₁₋₆ haloalkyl, C₁₋₆ haloalkoxy, halogen, —OR¹¹⁴, or —N(R¹¹⁴)₂;
each R¹¹³ is independently H, C₁₋₆ alkyl, C₁₋₆ alkoxy, C₁₋₆ haloalkyl, or phenyl, wherein the haloalkyl is substituted with 0 or 1 N(R¹¹⁴)₂;
each R¹¹⁴ is independently H or C₁₋₆ alkyl;
   each R¹¹⁶ is —OR¹¹⁷, —N(R¹¹⁷)₂, —N(R¹¹⁷)(CO)R¹¹⁷, —N(R¹¹⁷)(CO)OR¹¹⁷, —N(R¹¹⁷)S(O)₂R¹¹⁷, —(CO)R¹¹⁷, —SO₂R¹¹⁷, —SO₂N(R¹¹⁷)₂, or R³⁰⁰;
   each R¹¹⁵ and R¹¹⁷ is independently H, C₁₋₆ alkyl, C₁₋₆ haloalkyl, phenyl, or R³⁰⁰
   R²⁰⁰ is —OR²⁰¹ or —N(R²⁰¹)₂;
   R²⁰¹ is H, C₁₋₆ alkyl, phenyl, or heteroaryl, wherein the phenyl or heteroaryl is substituted with 0, 1, or 2 —OR²⁰², —N(R²⁰²)₂ or R³⁰⁰;
   R²⁰² is H or C₁₋₆ alkyl; and
   R³⁰⁰ has one of the following structures:

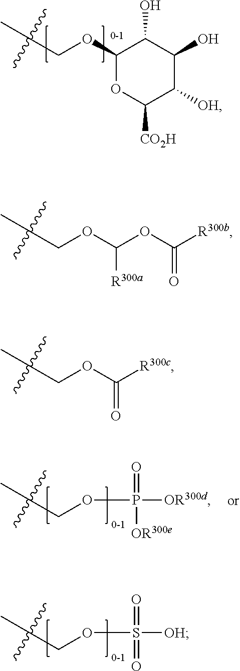

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl; and
   R^{300e} is H or C₁₋₆ alkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In certain embodiments, R²⁰⁰ is —OR²⁰¹ and R²⁰¹ has one of the following structures:

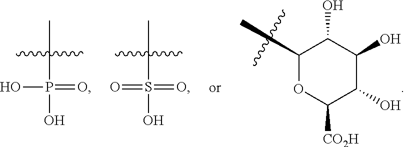

In some embodiments, R²⁰⁰ is —OR²⁰¹ and R²⁰¹ has the following structure:

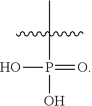 [SMILES: *P(=O)(O)O]

In some embodiments, R²⁰⁰ is —OR²⁰¹ and R²⁰¹ has the following structure:

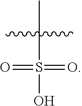 [SMILES: *S(=O)(=O)O]

In some embodiments, R²⁰⁰ is —OR²⁰¹ and R²⁰¹ has the following structure:

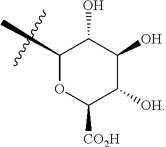

In some embodiments, the compound of the present disclosure is a compound of Formula I or a pharmaceutically acceptable salt thereof,
wherein
R¹⁰¹, R¹⁰², R¹⁰³, and R¹⁰⁴ are each independently H or F;
R¹⁰⁵ is C₂₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 1, 2, or 3 R¹⁰⁷, the alkynyl is substituted with 0, 1, 2, or 3 R¹⁰⁸, the phenyl is substituted with 2 or 3 R¹⁰⁹, and the -alkylene-phenyl, heteroaryl, -alkylene-heteroaryl, cycloalkyl or heterocyclyl is substituted with 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl or heterocyclyl is substituted with 0, 1, 2, or 3 R¹¹⁰;
each R¹⁰⁷ is independently C₂₋₆ alkoxyalkyl, C₁₋₆ haloalkoxy, phenyl, heteroaryl, C₃₋₈ cycloalkyl, heterocyclyl, or halogen, wherein the phenyl is substituted with 1, 2, or 3 R¹¹¹, and the heteroaryl, cycloalkyl, or heterocyclyl is substituted with 0, 1, 2, or 3 R¹¹¹;
each R¹⁰⁸ is independently C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹²;
each R¹⁰⁹ is independently C₁₋₆ alkyl, C₂₋₆ alkenyl, C₂₋₆ alkynyl, C₂₋₆ alkoxyalkyl, C₁₋₆ haloalkyl, halogen, —N₃, —OR¹¹³, or —N(R¹¹³)₂, wherein the alkyl, alkenyl, or alkynyl is substituted with 0 or 1 —S(O)₂(C₁₋₆ alkyl);
each R¹¹⁰ is independently C₂₋₆ alkyl, C₂₋₆ alkenyl, C₂₋₆ alkynyl, C₂₋₆ alkoxyalkyl, —(C₁₋₆ alkylene)-OR¹¹⁴, —(C₁₋₆ alkylene)-N(R¹¹⁴)₂, C₁₋₆ haloalkyl, phenyl, —(C₁₋₆ alkylene)-phenyl, heteroaryl, —(C₁₋₆ alkylene)-heteroaryl, halogen, —N₃, —OR¹¹⁵, —N(R¹¹⁵)₂, —N(R¹¹¹⁵)(CO)R¹¹⁵, —N(R¹¹⁵)(CO)OR¹¹⁵, —N(R¹¹⁵)S(O)₂R¹¹⁵, —(CO)R¹¹⁵, —SO₂R¹¹⁵, or —SO₂N(R¹¹⁵)₂, wherein the phenyl, alkylene-phenyl, heteroaryl, or alkylene-heteroaryl is substituted with 0, 1, 2, or 3 R¹¹⁶;
each R¹¹¹ and R¹¹² is independently C₁₋₆ alkyl, C₁₋₆ alkoxy, C₁₋₆ haloalkyl, C₁₋₆ haloalkoxy, halogen, —OR¹¹⁴, or —N(R¹¹⁴)₂;
each R¹¹³ is independently H, C₁₋₆ alkyl, C₁₋₆ alkoxy, C₁₋₆ haloalkyl, or phenyl, wherein the haloalkyl is substituted with 0 or 1 N(R¹¹⁴)₂;
each R¹¹⁴ is independently H or C₁₋₆ alkyl;
each R¹¹⁶ is —OR¹¹⁷, —N(R¹¹⁷)₂, —N(R¹¹¹⁷)(CO)R¹¹⁷, —N(R¹¹¹⁷)(CO)OR¹¹⁷, —N(R¹¹⁷)S(O)₂R¹¹⁷, —(CO)R¹¹⁷, —SO₂R¹¹⁷, or —SO₂N(R¹¹⁷)₂;
each R¹¹⁵ and R¹¹⁷ is independently H, C₁₋₆ alkyl, C₁₋₆ haloalkyl, or phenyl;
R²⁰⁰ is —OR²⁰¹ or —N(R²⁰¹)₂;
R²⁰¹ is H, C₁₋₆ alkyl, phenyl, or heteroaryl, wherein the phenyl or heteroaryl is substituted with 0, 1, or 2 —OR²⁰² or —N(R²⁰²)₂;
R²⁰² is H or C₁₋₆ alkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments of the compound or pharmaceutically acceptable salt thereof, one of R¹⁰¹, R¹⁰², R¹⁰³, and R¹⁰⁴ is F, and three of R¹⁰¹, R¹⁰², R¹⁰³, and R¹⁰⁴ are H. In some embodiments, two of R¹⁰¹, R¹⁰², R¹⁰³, and R¹⁰⁴ are F, and two of R¹⁰¹, R¹⁰², R¹⁰³, and R¹⁰⁴ are H.

In some embodiments of the compound or pharmaceutically acceptable salt thereof, R¹⁰¹ is H. In some embodiments, R¹⁰¹ is F.

In some embodiments of the compound or pharmaceutically acceptable salt thereof, R¹⁰² is H. In some embodiments, R¹⁰² is F.

In some embodiments of the compound or pharmaceutically acceptable salt thereof, R¹⁰¹ is H, and R¹⁰² is F. In certain embodiments, R¹⁰¹ and R¹⁰² are each F. In some embodiments, R¹⁰¹ and R¹⁰² are each H. In some embodiments, R¹⁰¹ is F and R¹⁰² is H.

In some embodiments of the compound or pharmaceutically acceptable salt thereof, each R¹⁰³ and R¹⁰⁴ is H. In certain embodiments, R¹⁰³ and R¹⁰⁴ are each F. In some embodiments, R¹⁰³ is F and R¹⁰⁴ is H. In some embodiments, R¹⁰³ is H and R¹⁰⁴ is F. In some embodiments, R¹⁰³ and R¹⁰⁴ are both H and R¹⁰¹ and R¹⁰² are each H. In certain embodiments, R¹⁰³ and R¹⁰⁴ are both H and R¹⁰¹ and R¹⁰² are each F. In some embodiments, R¹⁰³ and R¹⁰⁴ are both H and R¹⁰¹ is F, and R¹⁰² is H. In certain embodiments, R¹⁰³ and R¹⁰⁴ are both H and R¹⁰¹ is F, and R¹⁰² is H.

In some embodiments of the compound or pharmaceutically acceptable salt thereof, R¹⁰⁵ is C₂₋₆ alkyl, C₂₋₆ alkenyl, C₂₋₆ alkynyl, or C₁₋₆ haloalkyl, wherein the alkyl or alkenyl is substituted with 1, 2, or 3 R¹⁰⁷, the alkynyl is substituted with 0, 1, 2, or 3 R¹⁰⁸. In some embodiments, R¹⁰⁵ is C₂₋₆ alkenyl, wherein the alkenyl is substituted with 1, 2, or 3 R¹⁰⁷.

In some embodiments of the compound or pharmaceutically acceptable salt thereof, R¹⁰⁵ is phenyl, —(C₁₋₆ alkylene)-phenyl, heteroaryl, —(C₁₋₆ alkylene)-heteroaryl, C₃₋₈ cycloalkyl, or heterocyclyl, wherein the phenyl is substituted with 2 or 3 R¹⁰⁹, and the -alkylene-phenyl, heteroaryl, -alkylene-heteroaryl, cycloalkyl or heterocyclyl is substituted with 1, 2 or 3 R¹¹⁰. In some embodiments, R¹⁰⁵ is phenyl, wherein the phenyl is substituted with 2 or 3 R¹⁰⁹. In some embodiments, R¹⁰⁵ is —(C₁₋₆ alkylene)-phenyl, heteroaryl, —(C₁₋₆ alkylene)-heteroaryl, C₃₋₈ cycloalkyl, or heterocyclyl, wherein the -alkylene-phenyl, heteroaryl, -alkylene-heteroaryl, cycloalkyl or heterocyclyl is substituted with 1, 2 or 3 R¹¹⁰. In some embodiments, R¹⁰⁵ is C₃₋₈ cycloalkyl or heterocyclyl, wherein the cycloalkyl or heterocyclyl is substituted with 1, 2 or 3 R¹¹⁰. In some embodiments, R¹⁰⁵ is heteroaryl or —(C₁₋₆ alkylene)-heteroaryl, wherein the heteroaryl or -alkylene-heteroaryl is substituted with 1, 2 or 3 R¹¹⁰.

In some embodiments of the compound or pharmaceutically acceptable salt thereof, R¹⁰⁵ is heteroaryl, wherein the heteroaryl is substituted with 1, 2 or 3 R¹¹⁰. In some embodiments, R¹⁰⁵ is thienyl, imidazolyl, triazolyl, indolyl, indazolyl, or thienothienyl, which is substituted with 0, 1, 2, or 3 R¹¹⁰. In some embodiments, R¹⁰⁵ is thienyl, which is substituted with 0, 1, 2, or 3 R¹¹⁰. In some embodiments, R¹⁰⁵ is thienyl, which is substituted with 0, 1, or 2 R¹¹⁰.

In some embodiments of the compound or pharmaceutically acceptable salt thereof, R¹⁰⁵ is

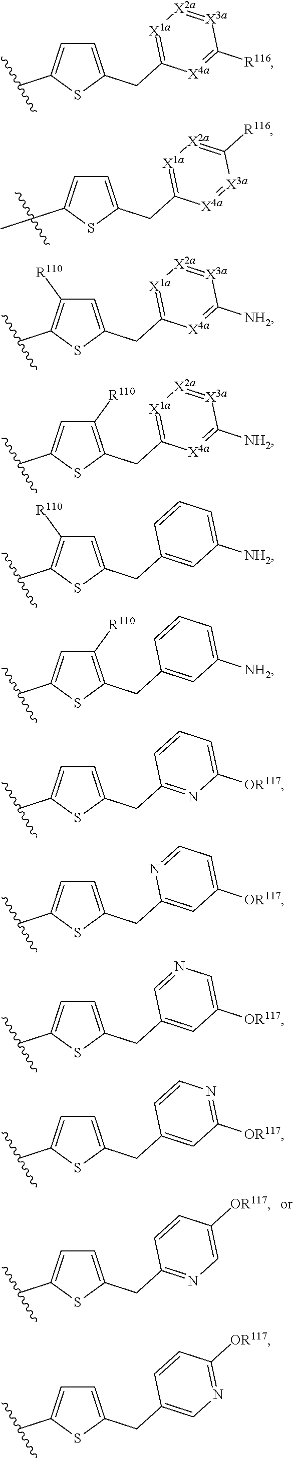

wherein
   each X^{1a}, X^{2a}, X^{3a}, and X^{4a} is independently CH or N;
   R¹¹⁰ is CH₃, CH₂F, CHF₂, or CF₃;
   R¹¹⁶ is —NH(CO)CH₃ or —NHS(O)₂CH₃; and
   R¹¹⁷ is CH₃, CH₂F, CHF₂, or CF₃.

In some embodiments of the compound or pharmaceutically acceptable salt thereof, R¹⁰⁵ is

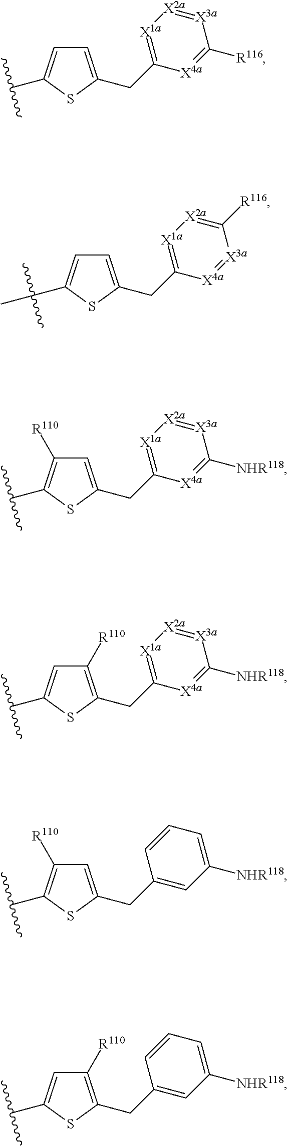

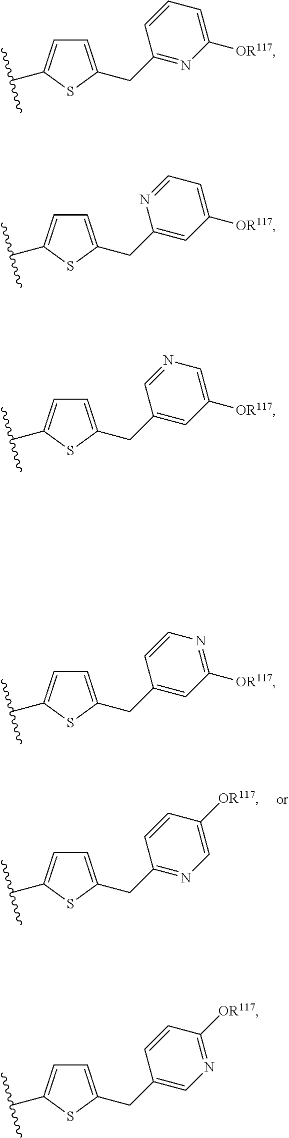

each X^{1a}, X^{2a}, X^{3a}, and X^{4a} is independently CH or N;
R¹¹⁰ is CH₃, CH₂F, CHF₂, or CF₃;
R¹¹⁶ is —NH(CO)CH₃, —NHS(O)₂CH₃ or R³⁰⁰;
R¹¹⁷ is CH₃, CH₂F, CHF₂, CF₃, or R³⁰⁰;
R¹¹⁸ is H or R³⁰⁰; and
R³⁰⁰ has one of the following structures:

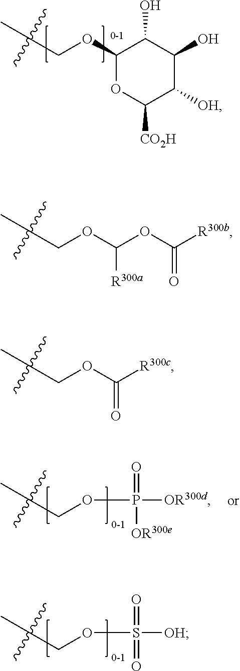

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl; and
   R^{300e} is H or C₁₋₆ alkyl.

In some embodiments of the compound or pharmaceutically acceptable salt thereof, R¹⁰⁵ is

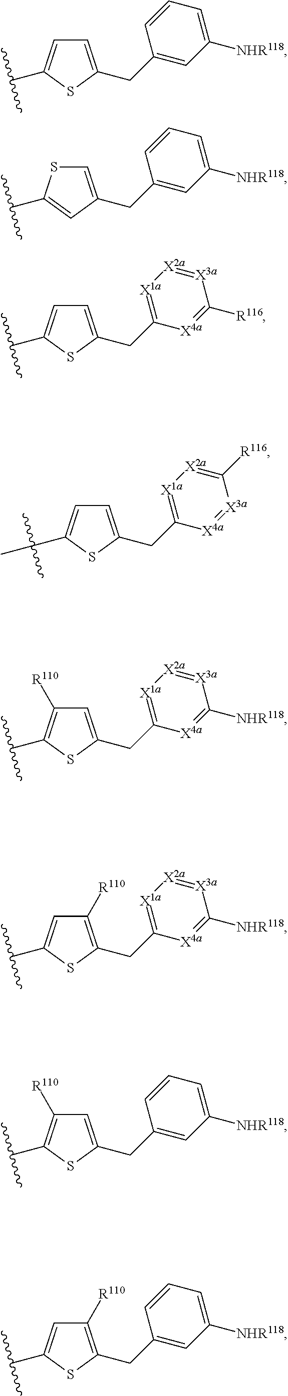

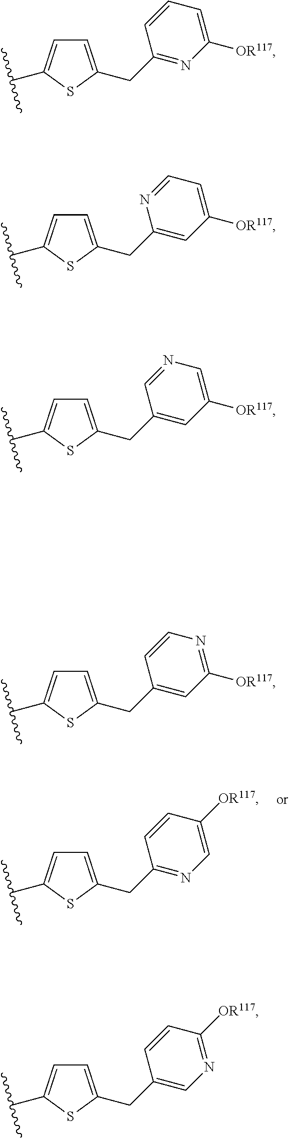

each X^{1a}, X^{2a}, X^{3a}, and X^{4a} is independently CH or N; R¹¹⁰ is CH₃, CH₂F, CHF₂, or CF₃;
R¹¹⁶ is —NH(CO)CH₃, —NHS(O)₂CH₃ or R³⁰⁰;
R¹¹⁷ is CH₃, CH₂F, CHF₂, CF₃, or R³⁰⁰;
R¹⁸ is H or R³⁰⁰; and
R³⁰⁰ has one of the following structures:

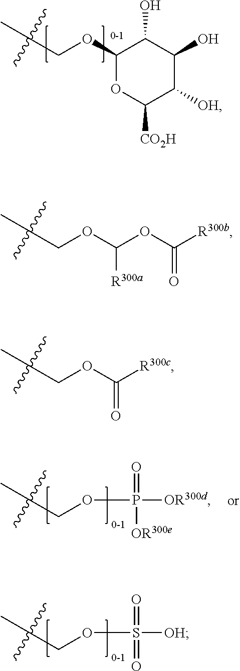

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl; and
   R³⁰⁰, is H or C₁₋₆ alkyl.

In some embodiments of the compound or pharmaceutically acceptable salt thereof, R¹⁰⁵ is

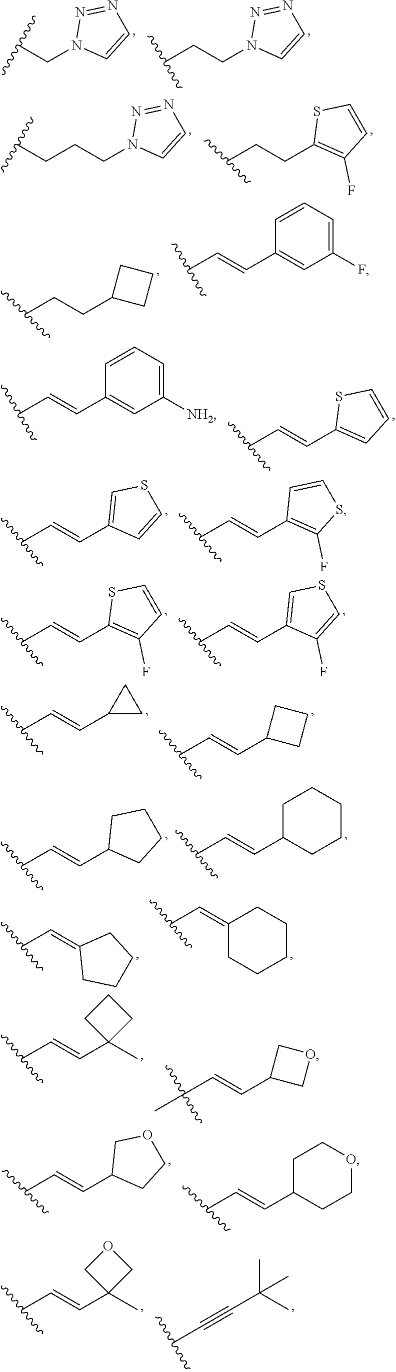

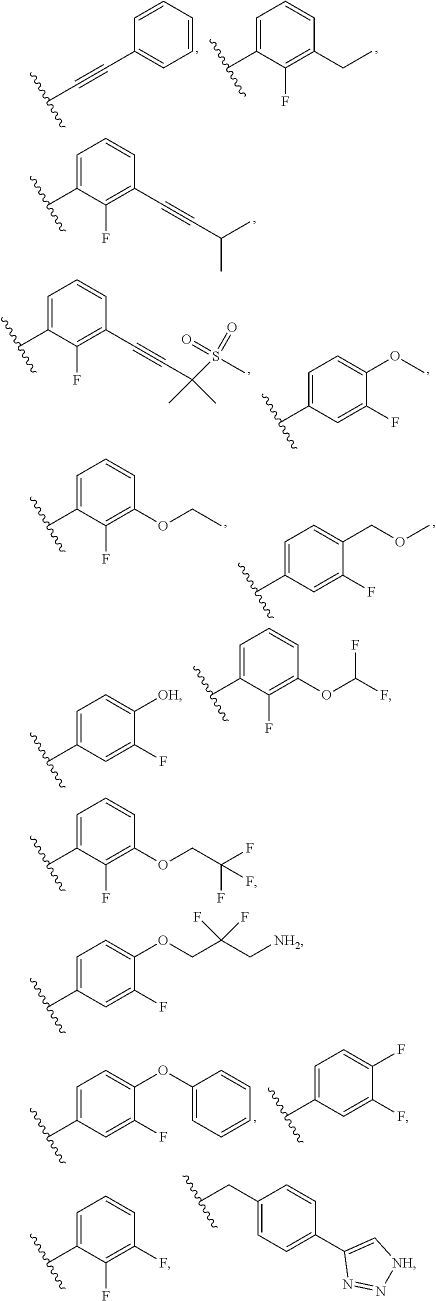

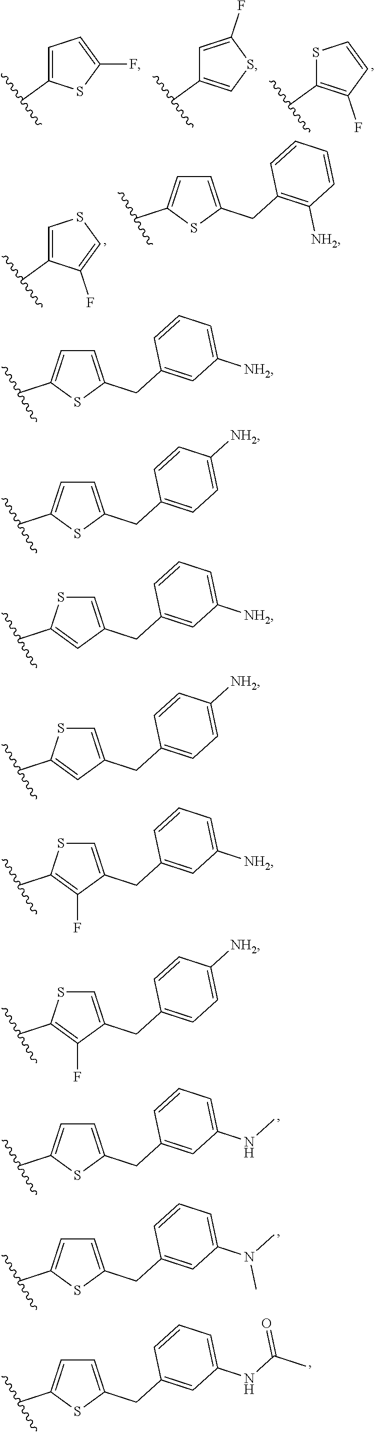

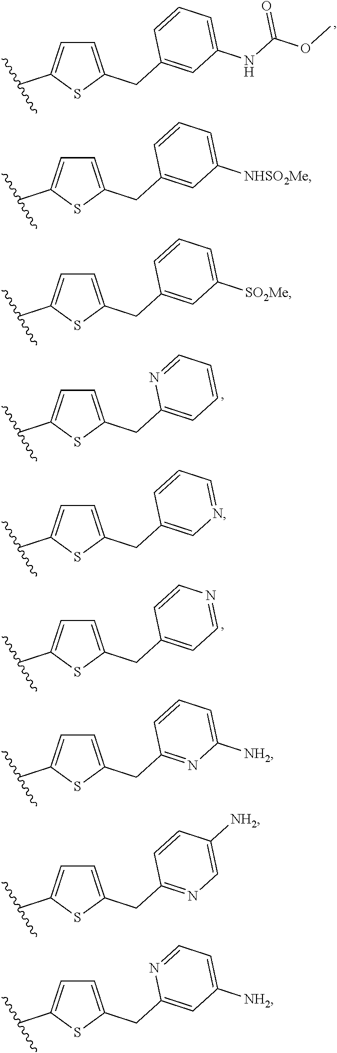

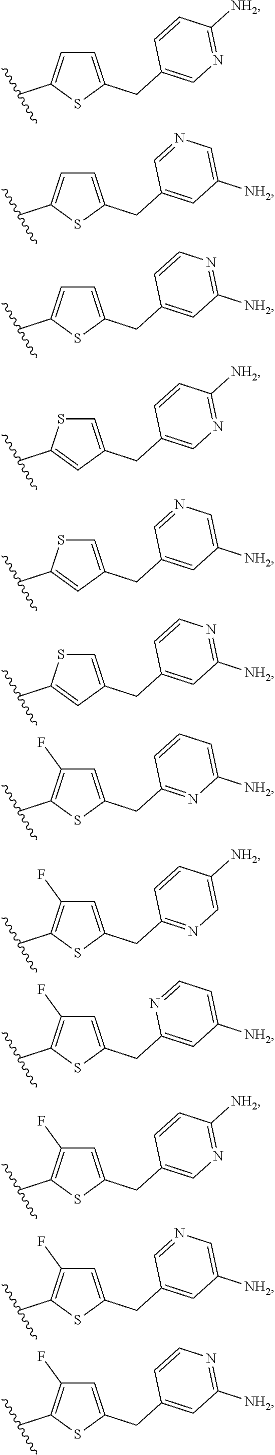

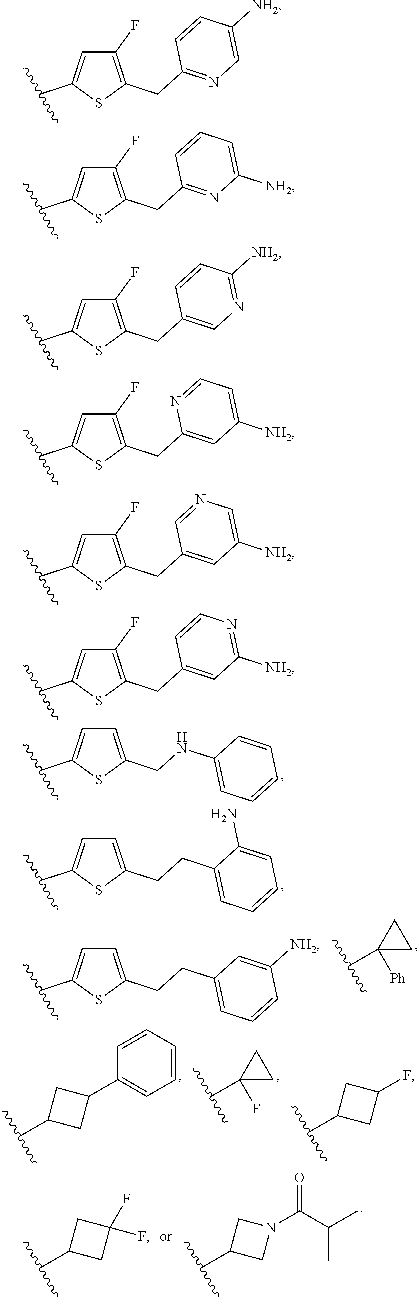

In some embodiments, the compound of the present disclosure is a compound of Formula II:

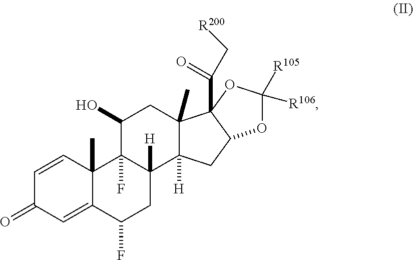

or a pharmaceutically acceptable salt thereof,
wherein
R¹⁰⁵ is C₄₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl is substituted with 1, 2, or 3 R¹¹⁰, and the heterocyclyl is substituted with 0, 1, 2 or 3 R¹¹⁰;
each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N3, —OR¹¹²,
   —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N3, —OR¹¹²,
   —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹¹ is independently C₁₋₆ alkyl, C₁₋₆ alkoxy, C₁₋₆ haloalkyl, C₁₋₆ haloalkoxy, or halogen;
each R¹¹² is independently H, C₁₋₆ alkyl, C₁₋₆ haloalkyl, or phenyl;
each R¹¹³ is independently —S(O)₂(C₁₋₆ alkyl) or —N(R¹¹⁵)₂;
each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶, or —S(O)₂N(R¹¹⁶)₂;
each R¹¹⁵ and R¹¹⁶ is independently H, C₁₋₆ alkyl, or C₁₋₆ haloalkyl;
R²⁰⁰ is —OR²⁰¹ or —N(R²⁰¹)₂;
R²⁰¹ is H, C₁₋₆ alkyl, phenyl, or heteroaryl, wherein the phenyl or heteroaryl is substituted with 0, 1, or 2 —OR²⁰² or —N(R²⁰²)₂;
R²⁰² is H or C₁₋₆ alkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, the compound of the present disclosure is a compound of Formula II:

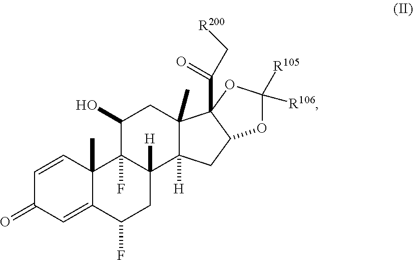

or a pharmaceutically acceptable salt thereof,
wherein
   R¹⁰⁵ is C₄₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
   R¹⁰⁶ is H;
   or, alternatively, R¹⁰⁵ and R¹⁰⁶ taken together combine to form a C₃₋₈ cycloalkyl or a heterocyclyl, wherein the cycloalkyl is substituted with 1, 2, or 3 R¹¹⁰, and the heterocyclyl is substituted with 0, 1, 2 or 3 R¹¹⁰;
   each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
   each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N3, —OR¹¹², —N(R¹¹²)₂, —(CO)R¹¹², or —S(O)₂R¹¹², wherein the alkynyl or haloalkoxy is substituted with 0, 1, 2, or 3 R¹¹³, and the phenyl, -alkylene-phenyl, heteroaryl, -alkylene-heteroaryl, or heterocyclyl is substituted with 0, 1, 2, or 3 R¹¹⁴;
   each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N3, —OR¹¹², —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
   each R¹¹⁰ is independently C₁₋₆ alkyl, C₁₋₆ alkoxy, C₁₋₆ haloalkyl, C₁₋₆ haloalkoxy, or halogen;
   each R¹¹² is independently H, C₁₋₆ alkyl, C₁₋₆ haloalkyl, or phenyl;
   each R¹¹³ is independently —S(O)₂(C₁₋₆ alkyl) or —N(R¹¹⁵)₂;
   each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶, —S(O)₂N(R¹¹⁶)₂ or R³⁰⁰;
   each R¹¹⁵ and R¹¹⁶ is, at each occurrence, independently H, C₁₋₆ alkyl, C₁₋₆ haloalkyl or R³⁰⁰;
   R²⁰⁰ is —OR²⁰¹ or —N(R²⁰¹)₂;
   R²⁰¹ is H, C₁₋₆ alkyl, phenyl, or heteroaryl, wherein the phenyl or heteroaryl is substituted with 0, 1, or 2 —OR²⁰², —N(R²⁰²)₂ or R³⁰⁰;
   R²⁰² is H or C₁₋₆ alkyl; and
   R³⁰⁰ has one of the following structures:

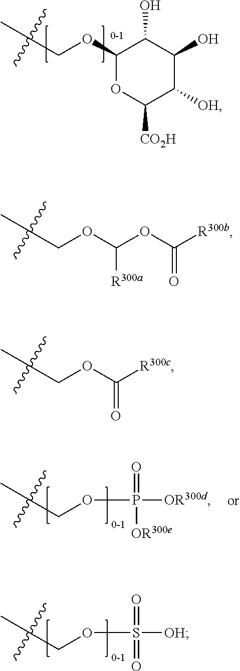

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl;
   R^{300e} is H or C₁₋₆ alkyl; and
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, R²⁰⁰ is —OR²⁰¹ and R²⁰¹ has the following structure:

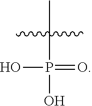 [SMILES: *P(=O)(O)O]

In some embodiments, R²⁰⁰ is —OR²⁰¹ and R²⁰¹ has the following structure:

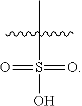 [SMILES: *S(=O)(=O)O]

In some embodiments, R²⁰⁰ is —OR²⁰¹ and R²⁰¹ has the following structure:

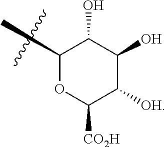

In some embodiments of the compound or pharmaceutically acceptable salt thereof, R¹⁰⁵ is C₄₋₆ alkyl, C₂₋₆ alkenyl, C₂₋₆ alkynyl, or C₁₋₆ haloalkyl, wherein the alkyl or alkenyl is substituted with 0, 1, 2, or 3 R¹⁰⁷, the alkynyl is substituted with 0, 1, 2, or 3 R¹⁰⁸. In some embodiments of the compound or pharmaceutically acceptable salt thereof, R¹⁰⁵ is C₂₋₆ alkenyl, wherein the alkenyl is substituted with 0, 1, 2, or 3 R¹⁰⁷.

In some embodiments of the compound or pharmaceutically acceptable salt thereof, each R¹⁰⁷ is independently phenyl, heteroaryl, C₃₋₈ cycloalkyl, heterocyclyl, or halogen, wherein the phenyl, heteroaryl, cycloalkyl, or heterocyclyl is substituted with 0, 1, or 2 R¹¹¹.

In some embodiments of the compound or pharmaceutically acceptable salt thereof, R¹⁰⁵ is phenyl, —(C₁₋₆ alkylene)-phenyl, heteroaryl, —(C₁₋₆ alkylene)-heteroaryl, C₃₋₈ cycloalkyl, or heterocyclyl, wherein the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰. In some embodiments, R¹⁰⁵ is heteroaryl or —(C₁₋₆ alkylene)-heteroaryl, wherein the heteroaryl or -alkylene-heteroaryl is substituted with 0, 1, 2 or 3 R¹¹⁰. In some embodiments, R¹⁰⁵ is thienyl, imidazolyl, triazolyl, indolyl, indazolyl, or thienothienyl, which is substituted with 0, 1, or 2 R¹¹⁰. In some embodiments, R¹⁰⁵ is thienyl, which is substituted with 0, 1, 2, or 3 R¹¹⁰. In some embodiments, R¹⁰⁵ is thienyl, which is substituted with 0, 1, or 2 R¹¹⁰.

In some embodiments of the compound or pharmaceutically acceptable salt thereof, each R¹¹⁰ is independently C₁₋₆ alkyl, C₂₋₆ alkenyl, C₂₋₆ alkynyl, C₂₋₆ alkoxyalkyl, phenyl, —(C₁₋₆ alkylene)-phenyl, heteroaryl, —(C₁₋₆ alkylene)-heteroaryl, heterocyclyl, halogen, —N₃, —OR¹¹², —N(R¹¹²)², —(CO)R¹¹², or —S(O)₂R¹¹², and the phenyl, alkylene-phenyl, heteroaryl, alkylene-heteroaryl, or heterocyclyl is substituted with 0, 1, 2, or 3 R¹¹⁴. In some embodiments, each R¹¹⁰ is independently C₁₋₃ alkyl or halogen.

In some embodiments of the compound or pharmaceutically acceptable salt thereof, R¹⁰⁵ is

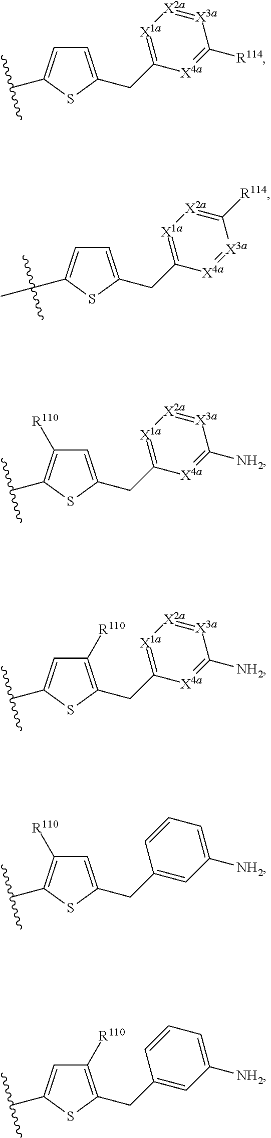

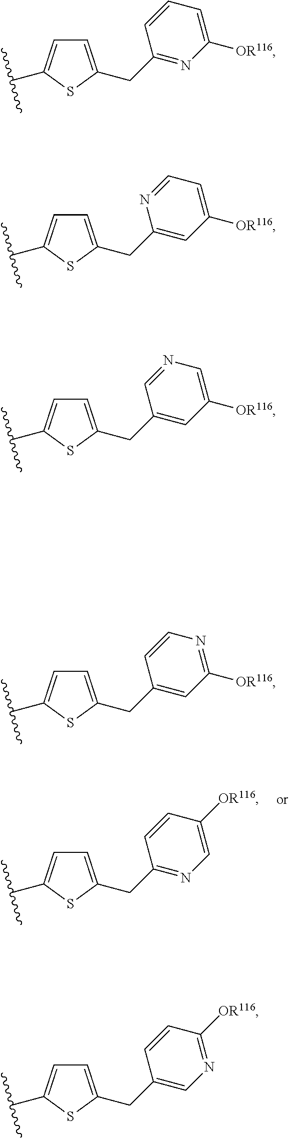

wherein
   each X^{1a}, X^{2a}, X^{3a}, and X^{4a} is independently CH or N;
   R¹¹⁰ is CH₃, CH₂F, CHF₂, or CF₃;
   R¹¹⁴ is —NH(CO)CH₃ or —NHS(O)₂CH₃; and
   R¹¹⁶ is CH₃, CHF₂, or CF₃.

In some embodiments of the compound or pharmaceutically acceptable salt thereof, R¹⁰⁵ is

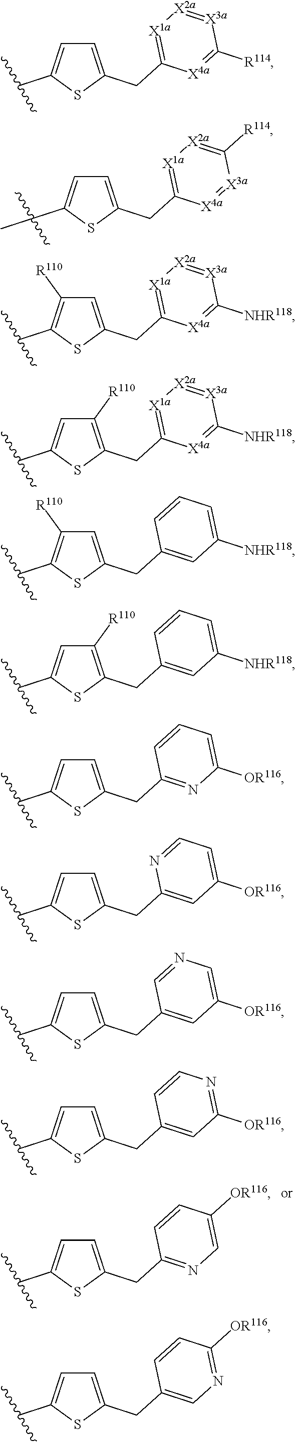

wherein
   each X^{1a}, X^{2a}, X^{3a}, and X^{4a} is independently CH or N;
   R¹¹⁰ is CH₃, CH₂F, CHF₂, or CF₃;
   R¹¹⁴ is —NH(CO)CH₃, —NHS(O)₂CH₃, or R³⁰⁰;
   R¹¹⁶ is CH₃, CH₂F, CHF₂, CF₃, or R³⁰⁰;
   R¹¹⁸ is H or R³⁰⁰; and
   R³⁰⁰ has one of the following structures:

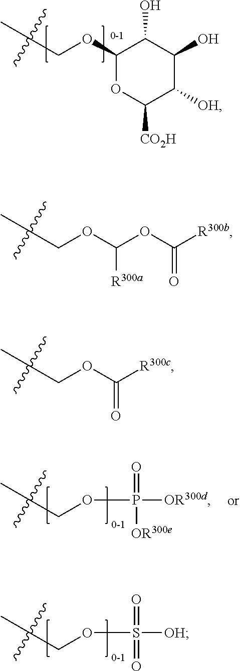

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl; and
   R^{300e} is H or C₁₋₆ alkyl.

In some embodiments of the compound or pharmaceutically acceptable salt thereof, R¹⁰⁵ is

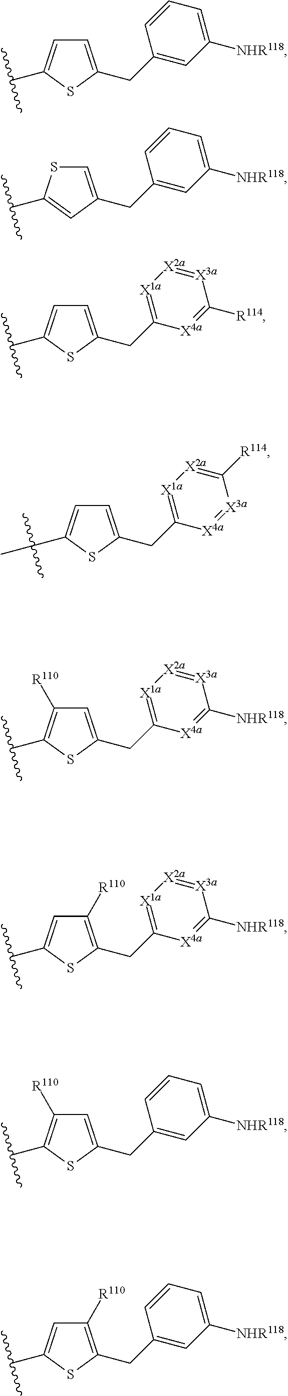

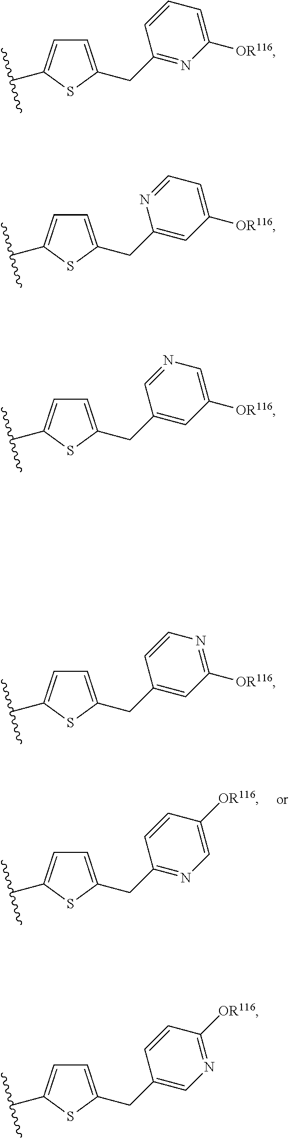

wherein
   each X^{1a}, X^{2a}, X^{3a}, and X^{4a} is independently CH or N;
   R¹¹⁰ is CH₃, CHF₂, or CF₃;
   R¹¹⁴ is —NH(CO)CH₃, —NHS(O)₂CH₃, or R³⁰⁰;
   R¹¹⁶ is CH₃, CH₂F, CHF₂, CF₃, or R³⁰⁰;
   R¹¹⁸ is H or R³⁰⁰; and
   R³⁰⁰ has one of the following structures:

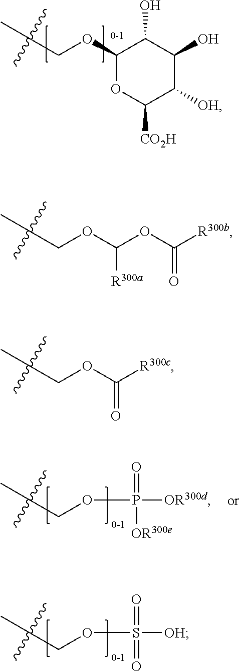

wherein:
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl; and
   R^{300e} is H or C₁₋₆ alkyl.

In some embodiments of the compound or pharmaceutically acceptable salt thereof, R¹⁰⁵ is

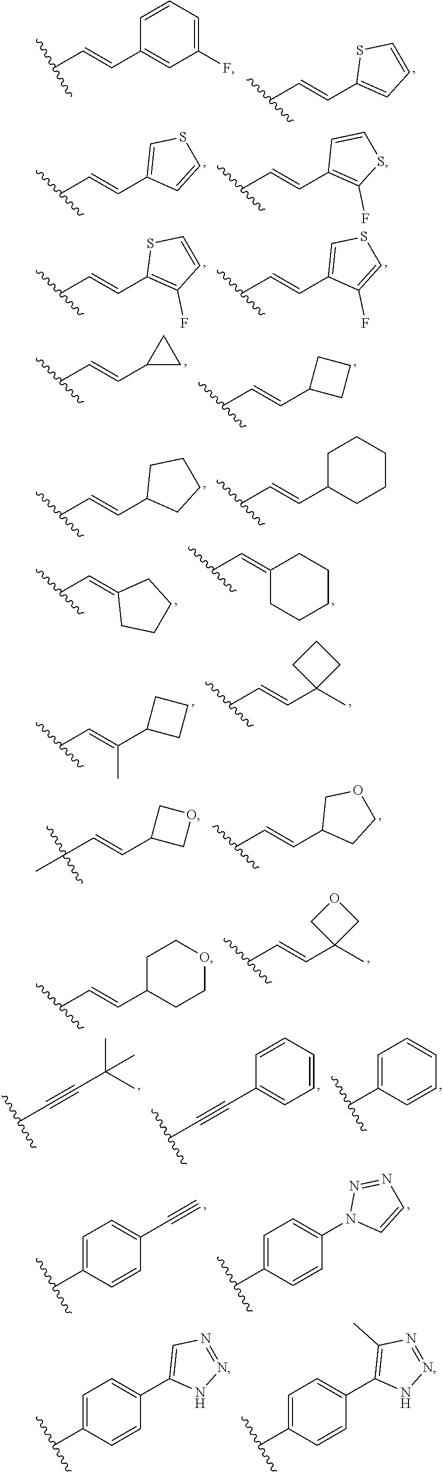

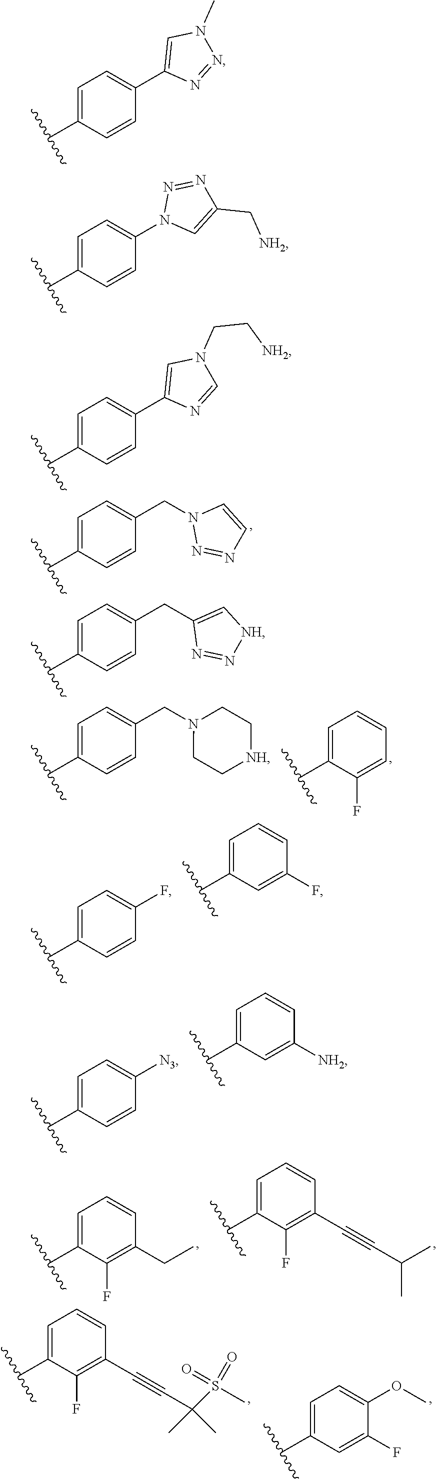

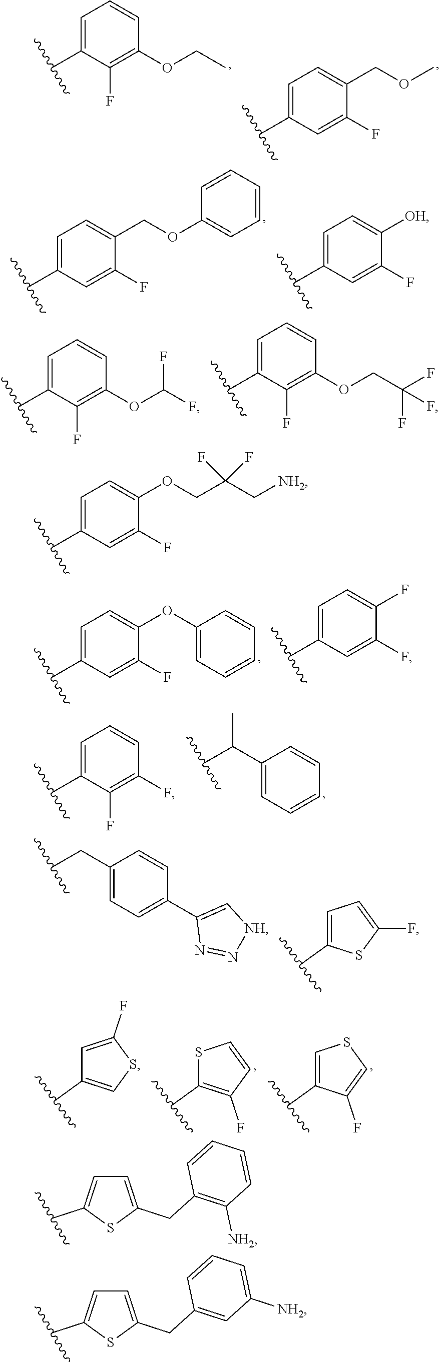

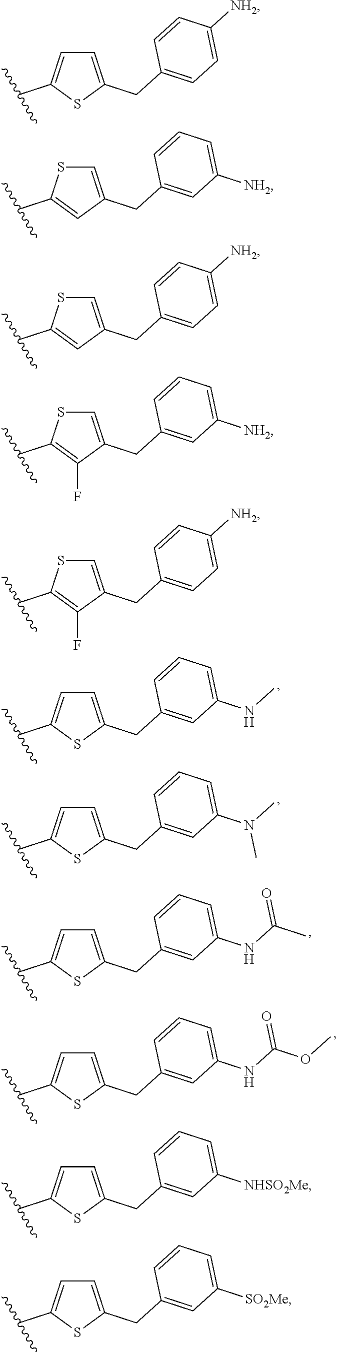

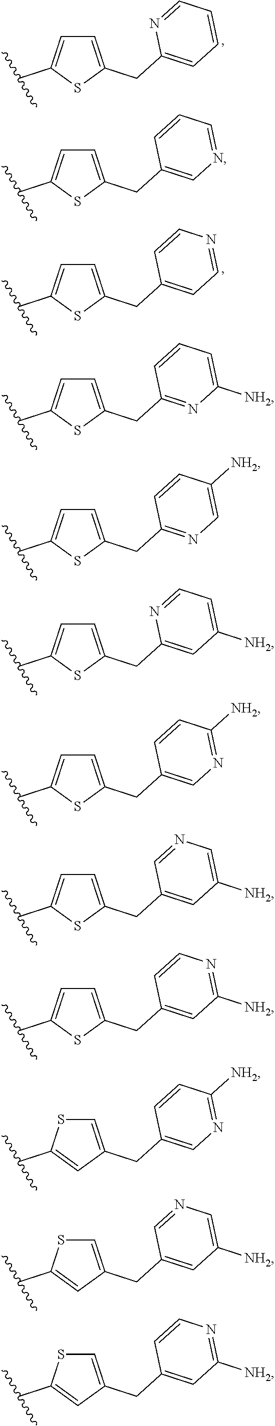

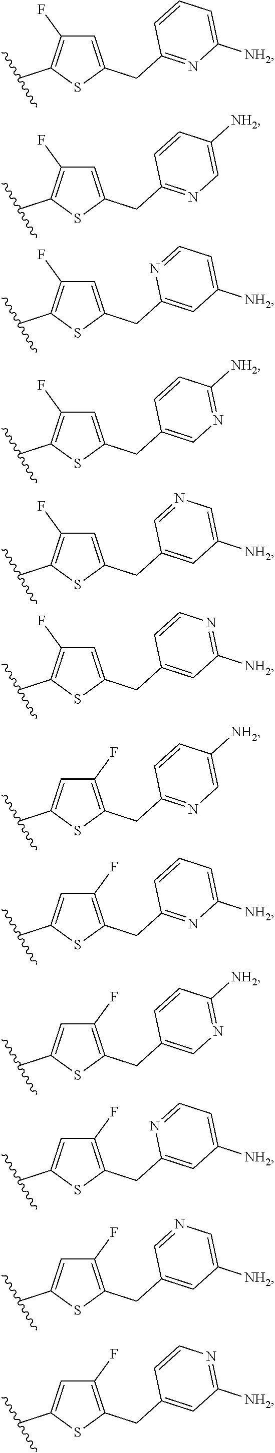

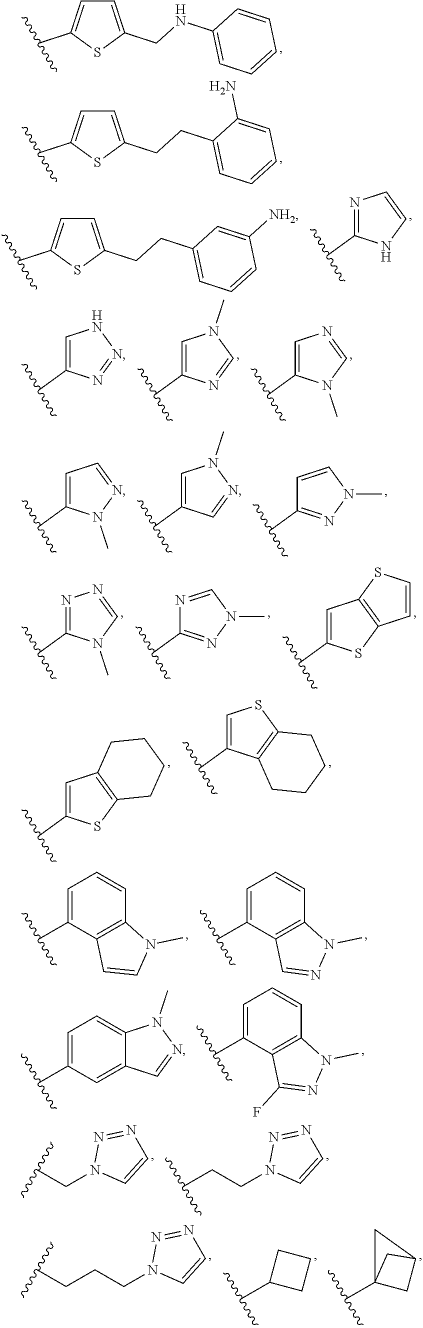

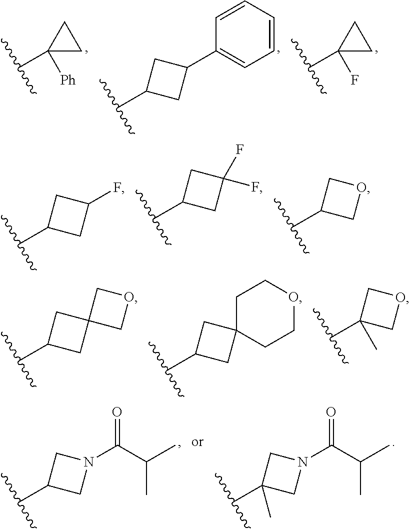

In some embodiments of the compound of the present disclosure or pharmaceutically acceptable salt thereof, such as a compound of Formula I or II, the heteroaryl in each instance is a 5- to 9-membered heteroaryl having 1 or 2 heteroatoms selected from N, O, and S. In some embodiments, the heteroaryl in each instance is a 5- to 6-membered heteroaryl having 1 or 2 heteroatoms selected from N, O, and S.

In some embodiments of the compound of the present disclosure or pharmaceutically acceptable salt thereof, such as a compound of Formula I or II, the heterocyclyl in each instance is a 4- to 9-membered heterocyclyl having 1 or 2 heteroatoms selected from N, O, and S. In some embodiments, the heterocyclyl in each instance is a 4- to 8-membered heterocyclyl having 1 or 2 heteroatoms selected from N, O, and S. In some embodiments, the heterocyclyl in each instance is a 4- to 6-membered heterocyclyl having 1 or 2 heteroatoms selected from N, O, and S.

In some embodiments of the compound of the present disclosure or pharmaceutically acceptable salt thereof, the compound has a structure of a compound described in the Examples herein.

In some embodiments of the compound of the present disclosure or pharmaceutically acceptable salt thereof, the compound has a structure selected from Table 1.

TABLE 1
Compounds

| Cmpd. | Structure/Name |
|---|---|
| 1 | 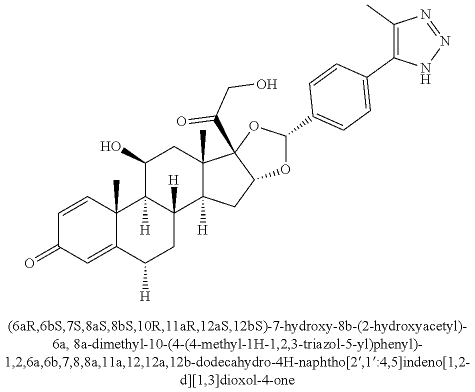 |
| 2 | 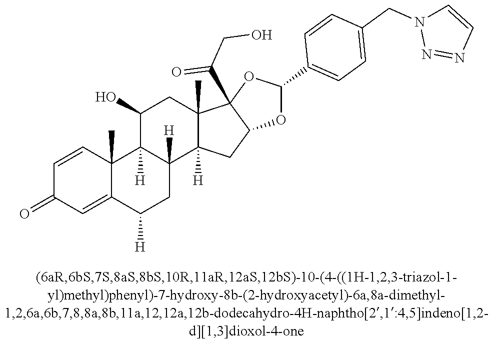 |
| 3 | 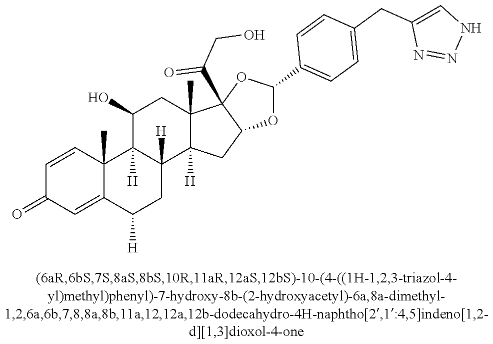 |
| 4 | 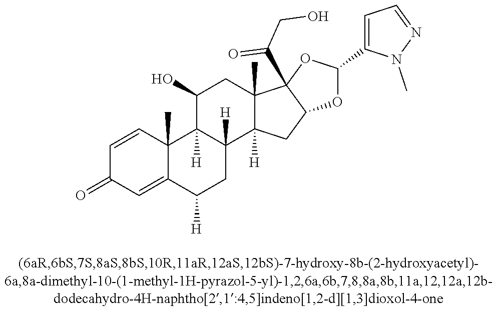 |
| 5 | 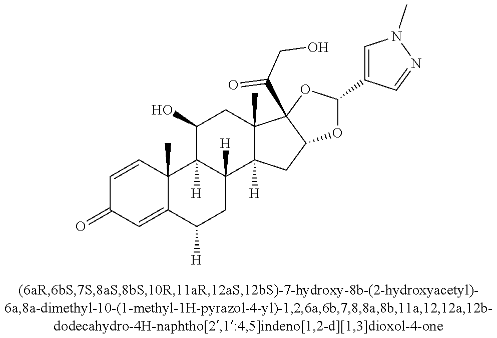 |
| 6 | 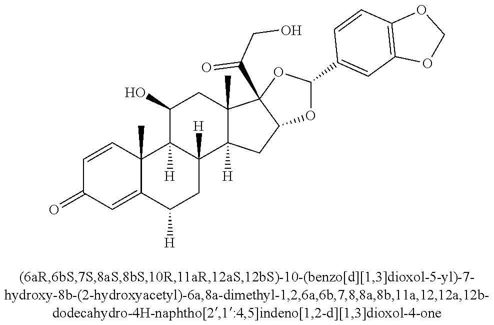 |
| 7 | 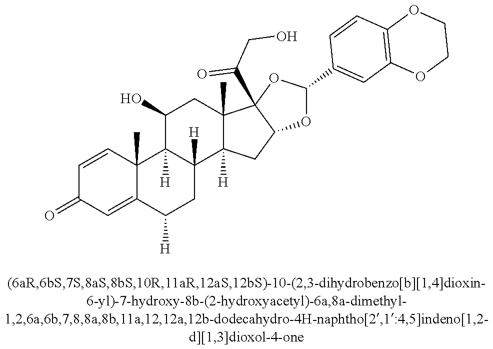 |
| 8 | 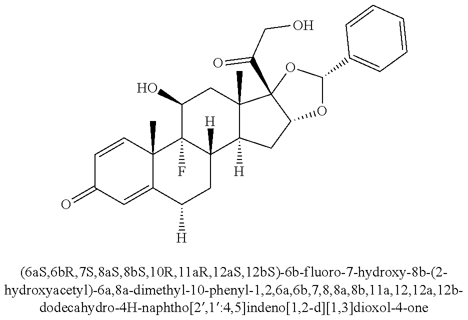 |
| 9 | 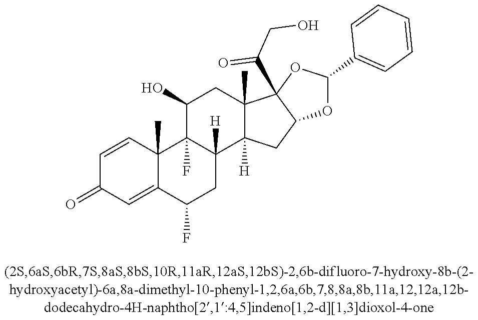 |
| 10 | 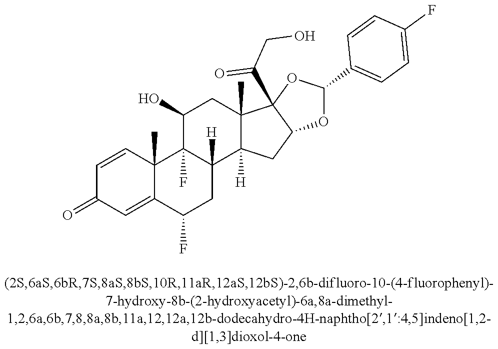 |
| 11 | 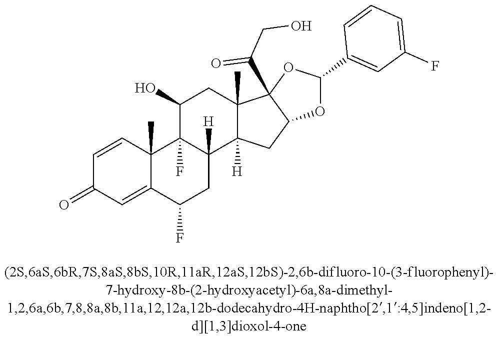 |
| 12 | 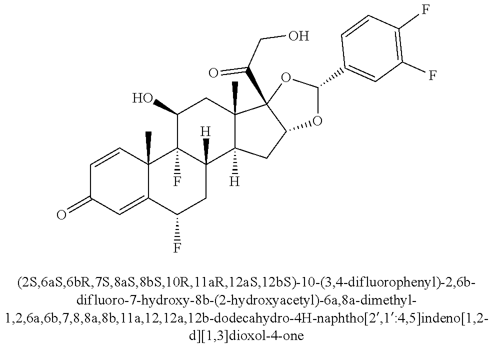 |
| 13 | 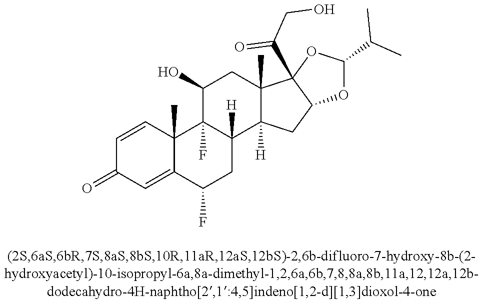 |
| 14 | 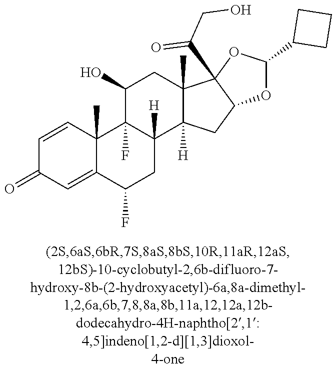 |
| 15 | 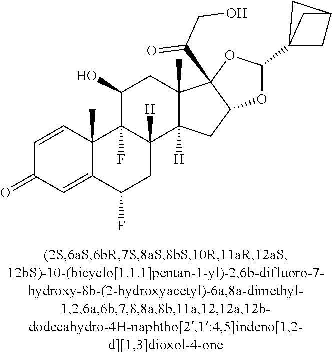 |
| 16 | 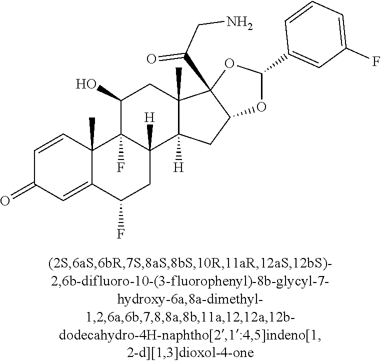 |
| 17 | 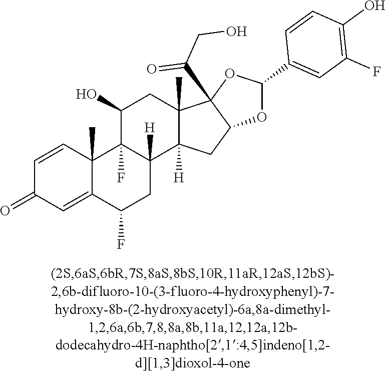 |
| 18 | 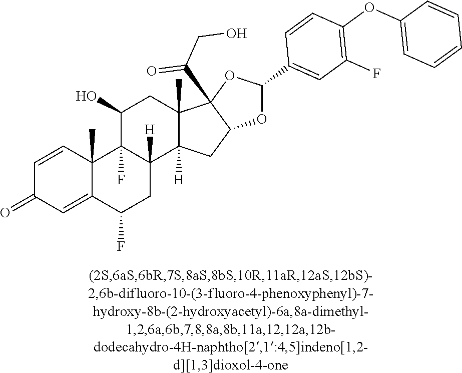 |
| 19 | 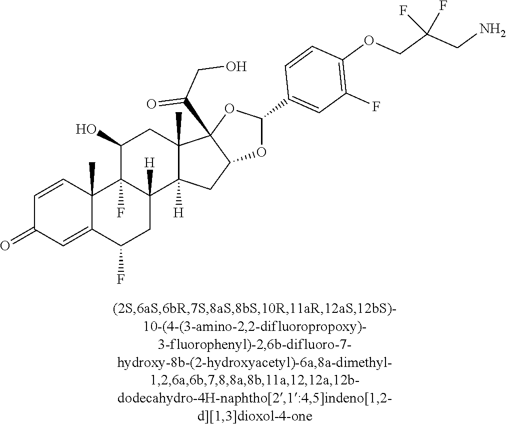 |
| 20 | 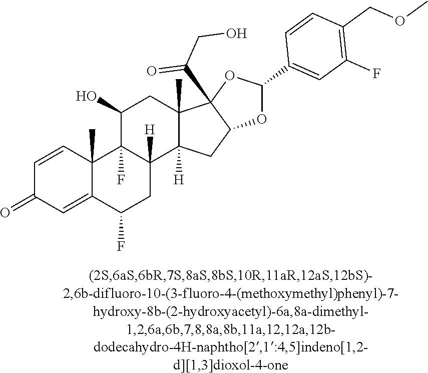 |
| 21 | 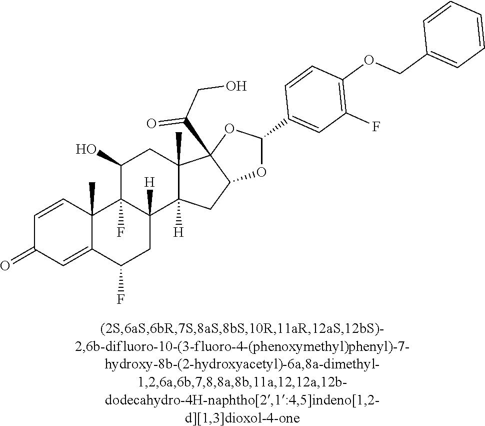 |
| 22 | 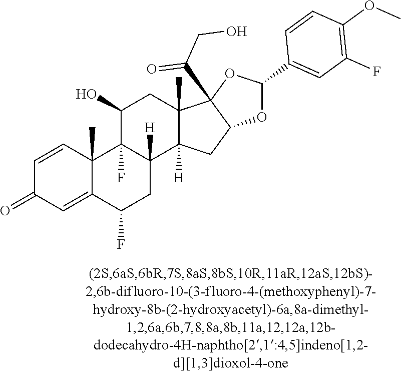 |
| 23 | 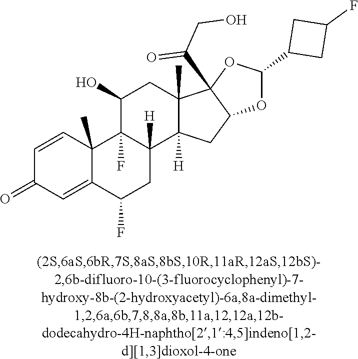 |
| 24 | 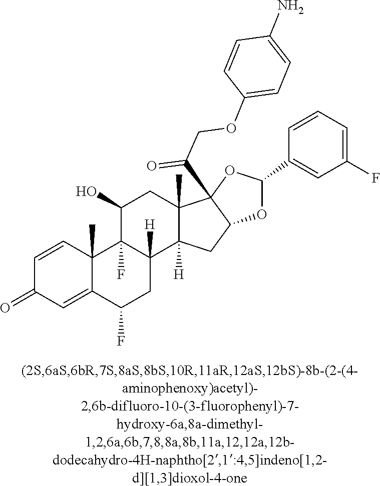 |
| 25 | 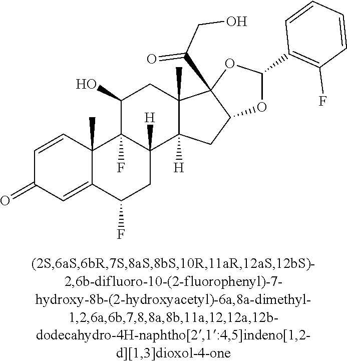 |
| 26 | 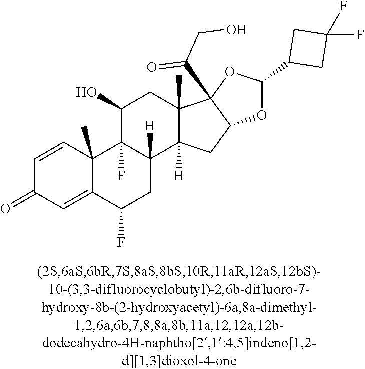 |
| 27 | 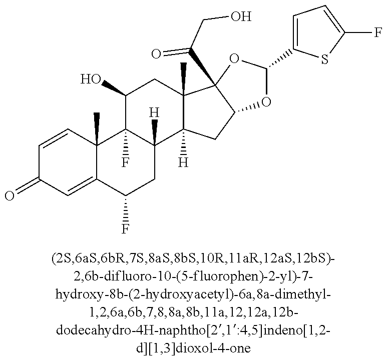 |
| 28 | 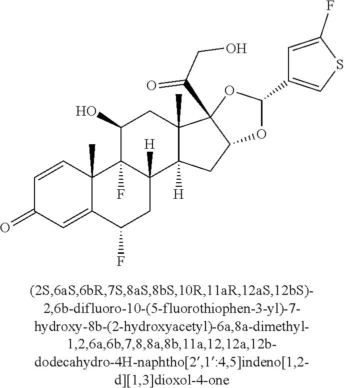 |
| 29 | 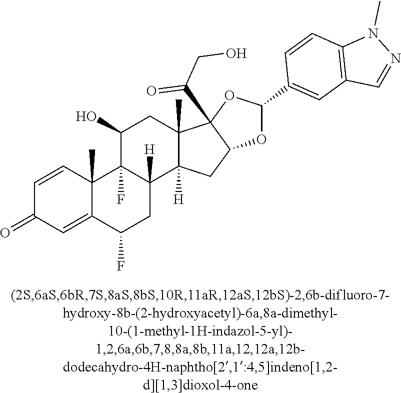 |
| 30a | 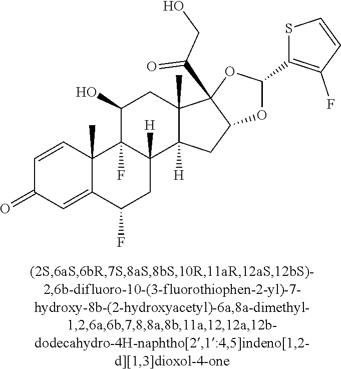 |
| 30b | 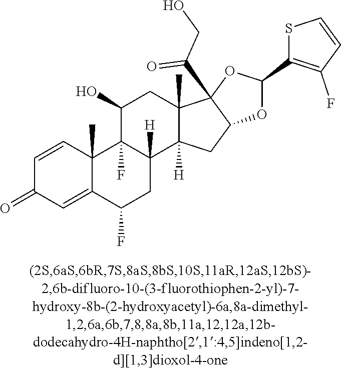 |
| 31 | 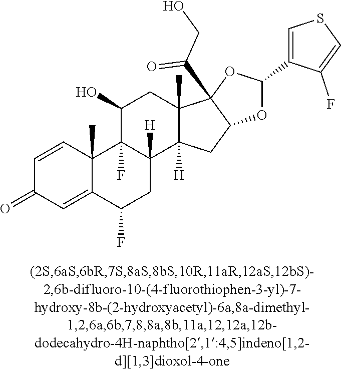 |
| 32 | 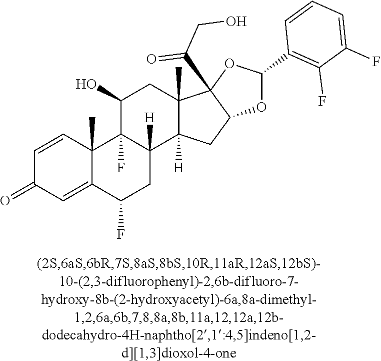 |
| 33 | 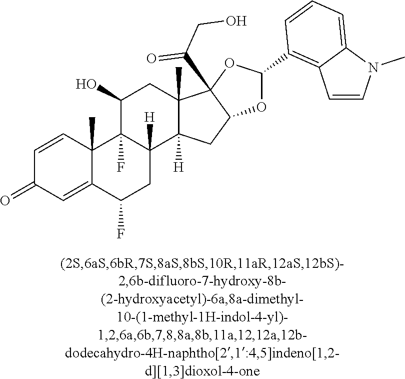 |
| 34 | 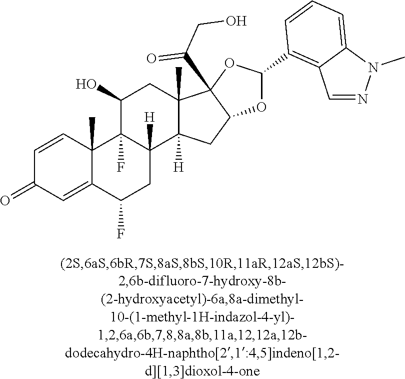 |
| 35 | 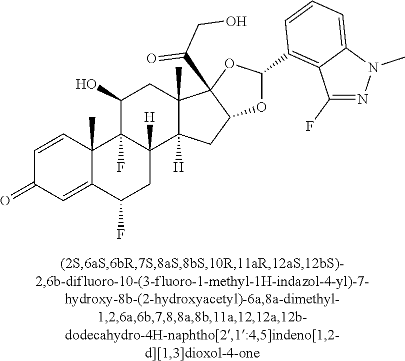 |
| 36 | 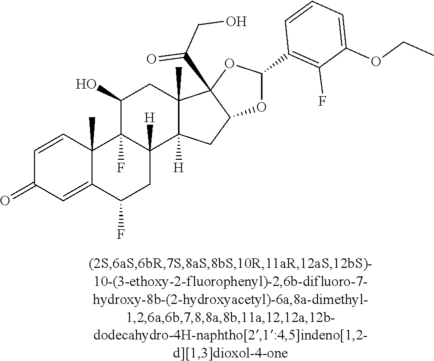 |
| 37 | 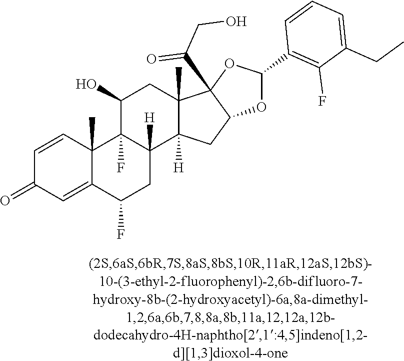 |
| 38 | 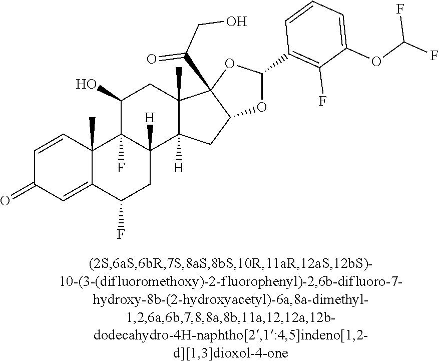 |
| 39 | 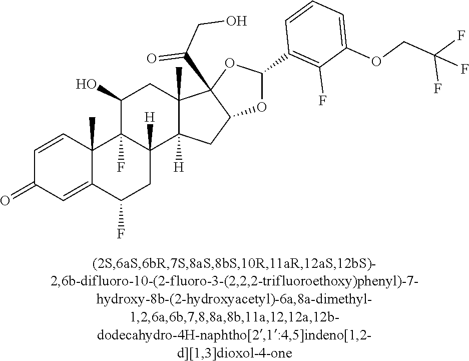 |
| 40 | 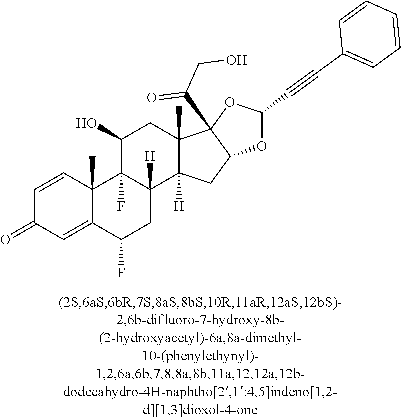 |
| 41 | 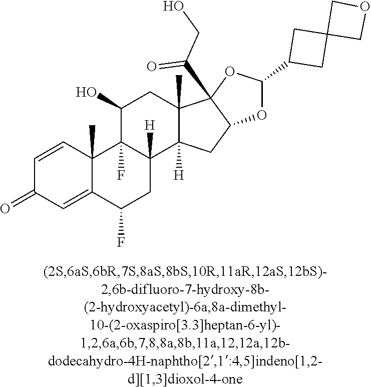 |
| 42 | 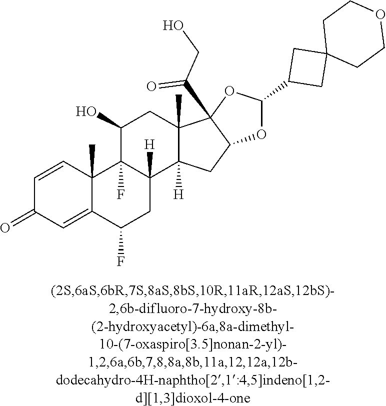 |
| 43 | 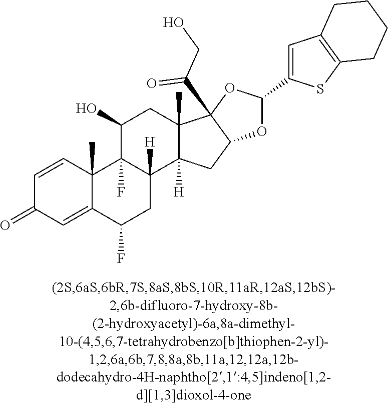 |
| 44 | 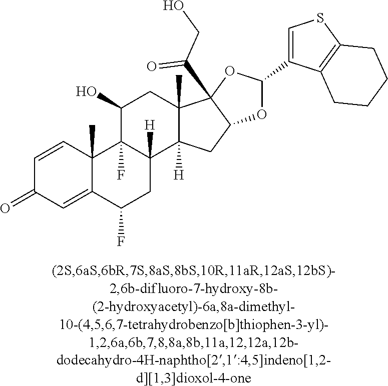 |
| 45 | 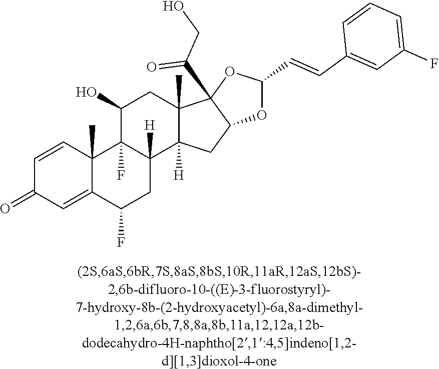 |
| 46 | 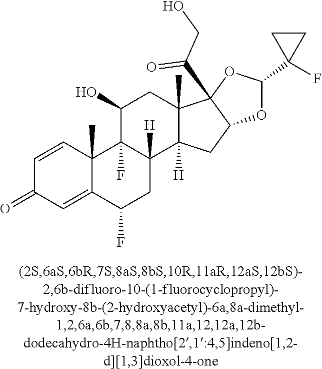 |
| 47 | 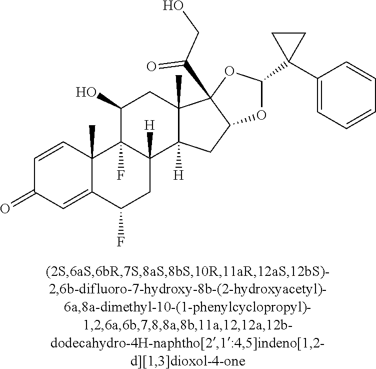 |
| 48 | 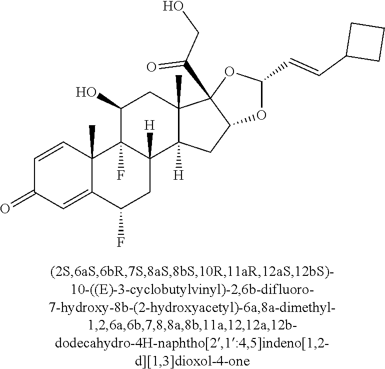 |
| 49 | 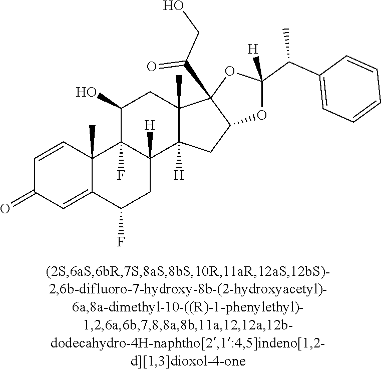 |
| 50 | 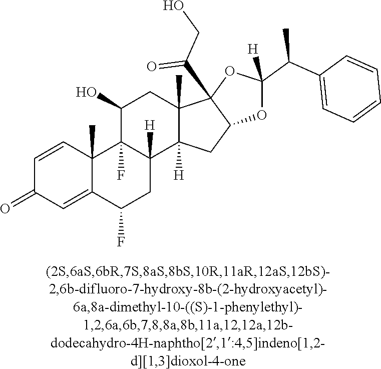 |
| 51 | 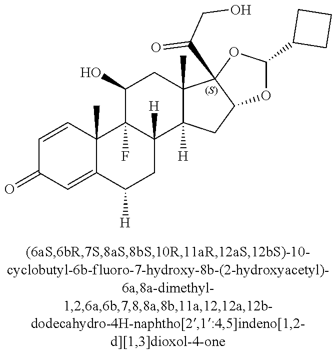 |
| 52a | 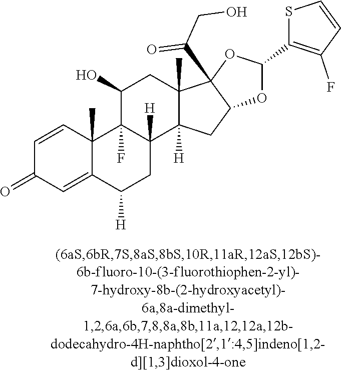 |
| 52b | 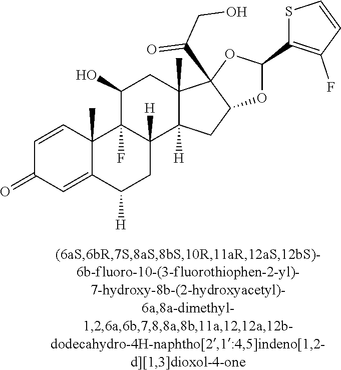 |
| 53a | 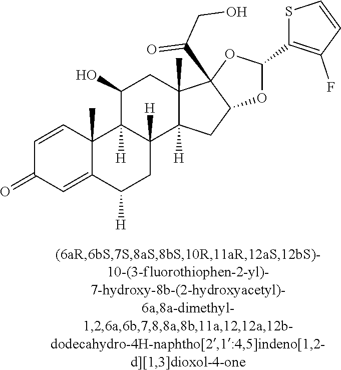 |
| 53b | 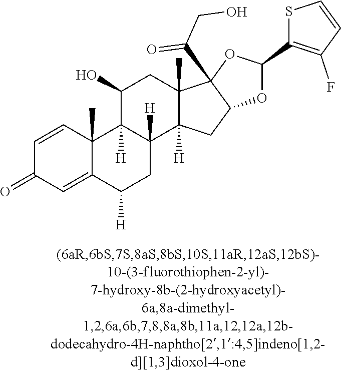 |
| 54 | 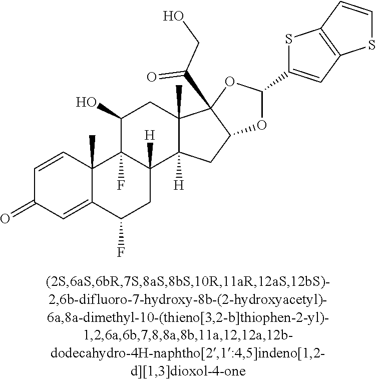 |
| 55 | 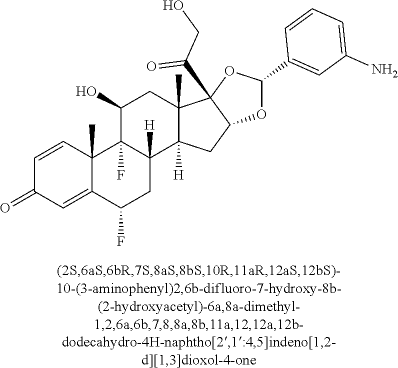 |
| 56 | 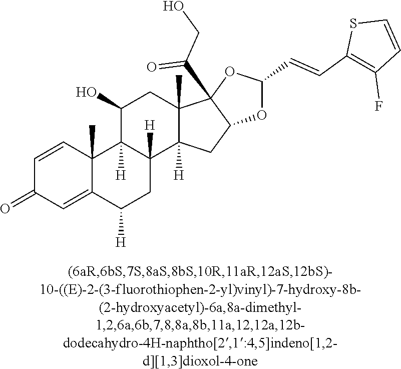 |
| 57 | 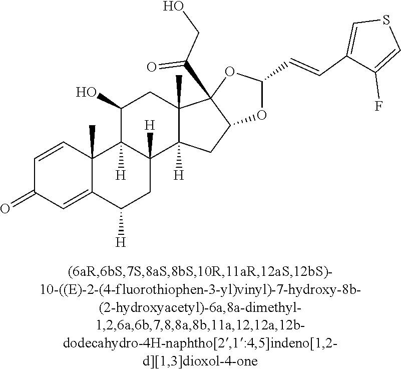 |
| 58 | 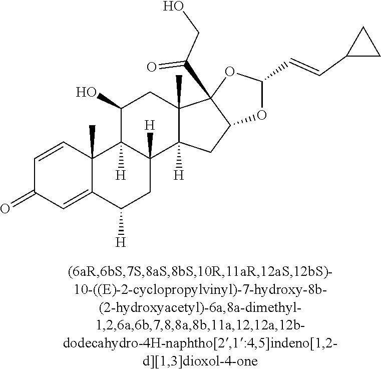 |
| 59 | 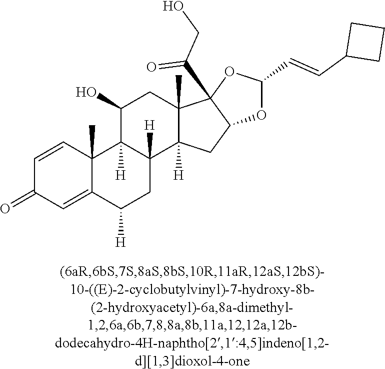 |
| 60 | 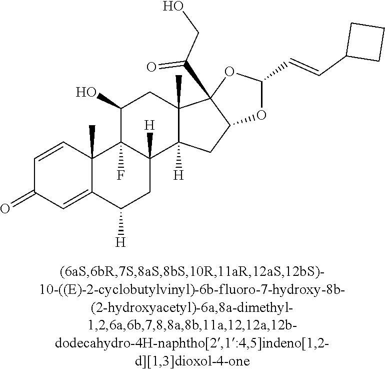 |
| 61a | 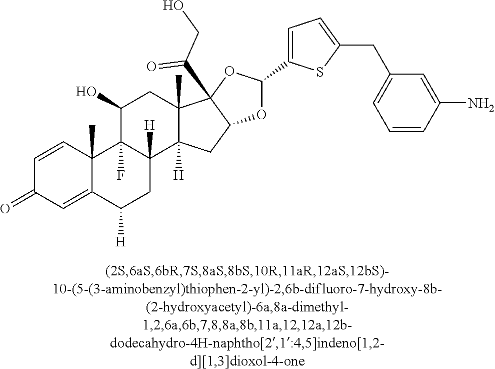 |
| 61b | 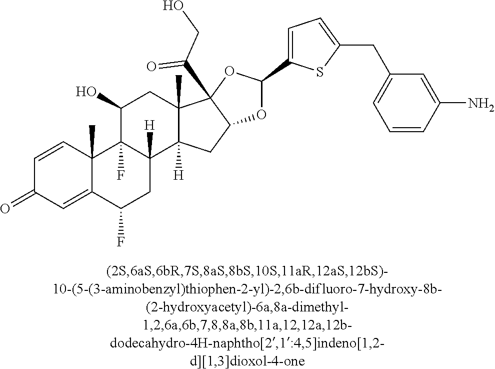 |
| 62a | 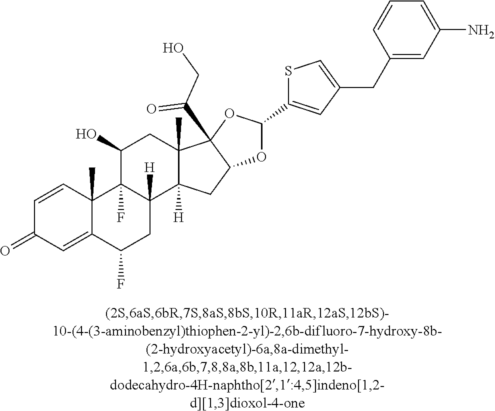 |
| 62b | 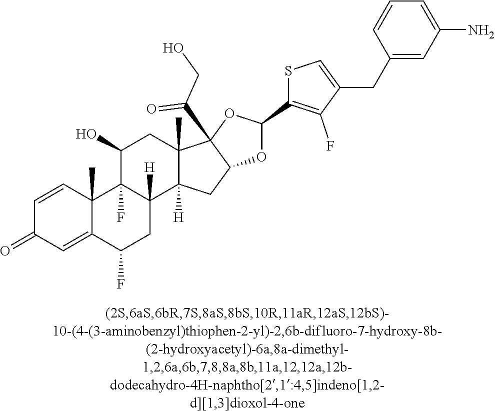 |
| 63 | 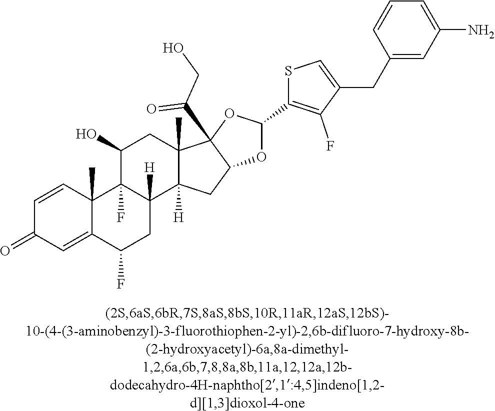 |
| 64 | 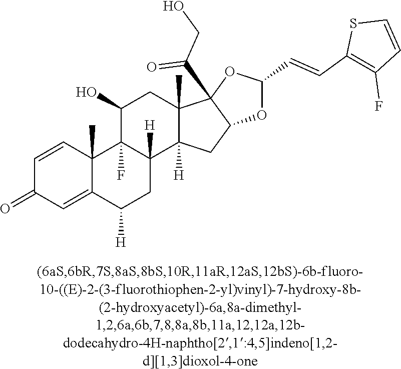 |
| 65 | 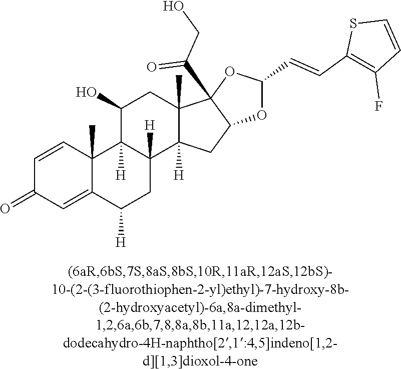 |
| 66 | 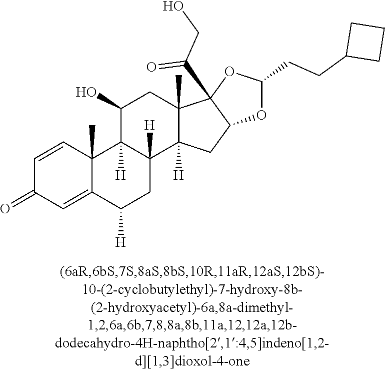 |
| 67 | 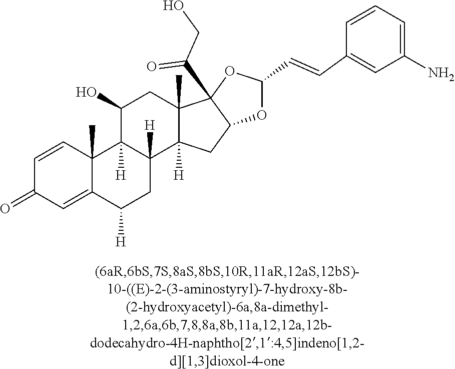 |
| 68 | 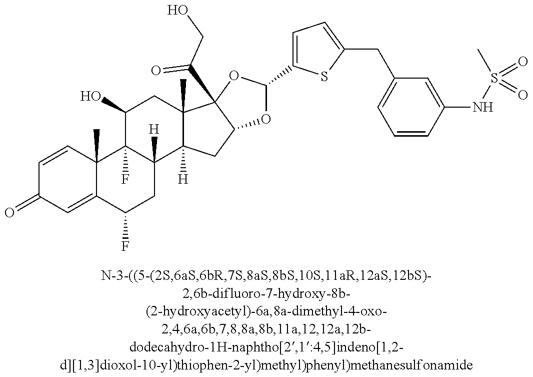 |
| 69 | 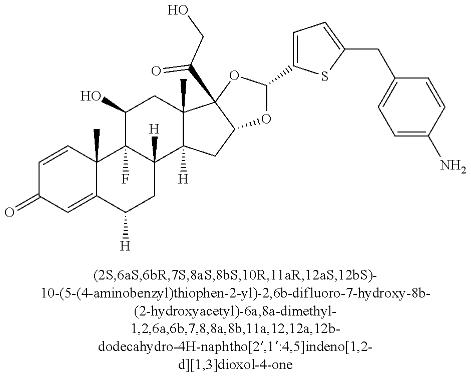 |
| 70 | 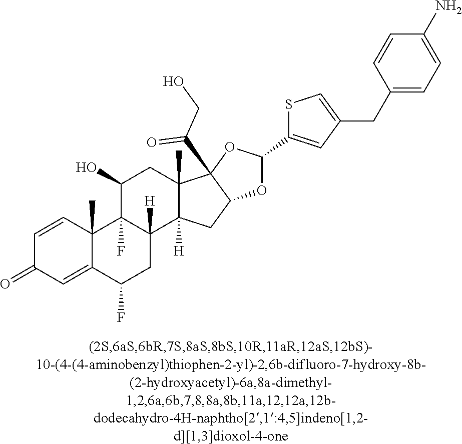 |
| 71 | 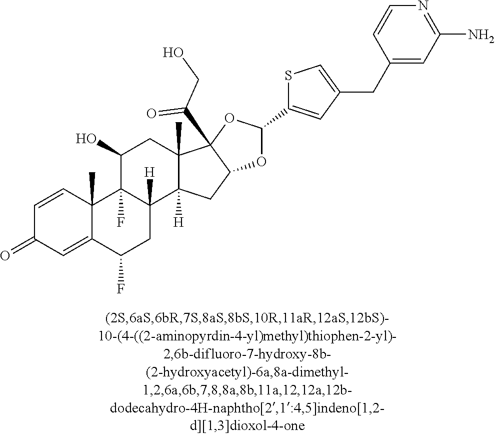 |
| 72 | 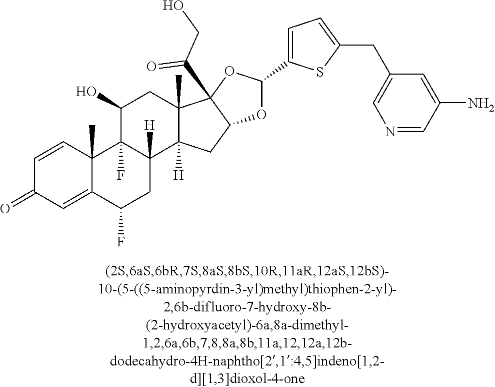 |
| 73 | 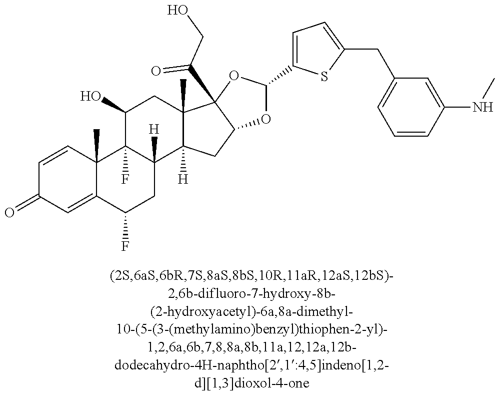 |
| 74 | 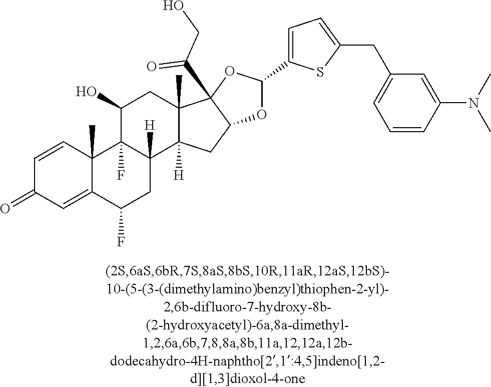 |
| 75 | 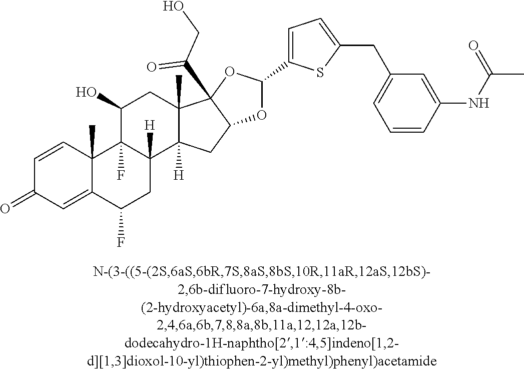 |
| 76 | 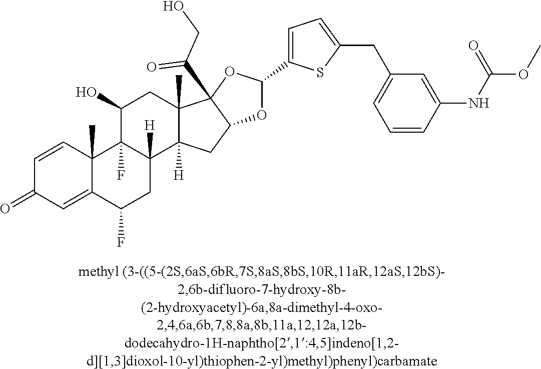 |
| 77 | 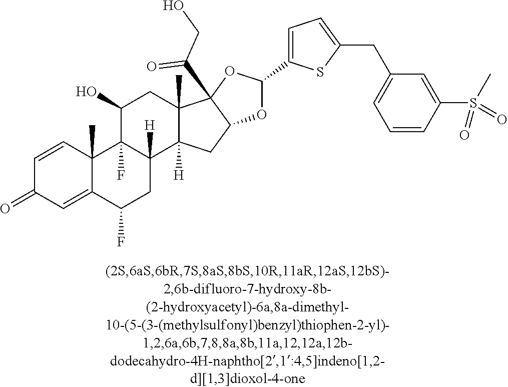 |
| 78 | 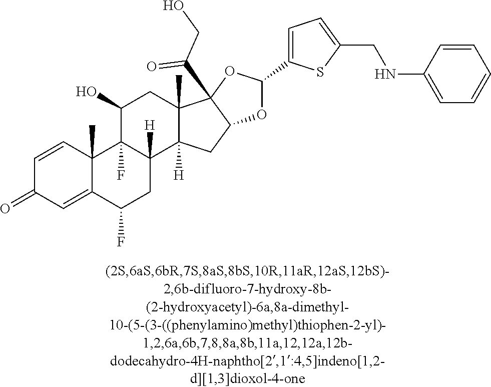 |
| 79 | 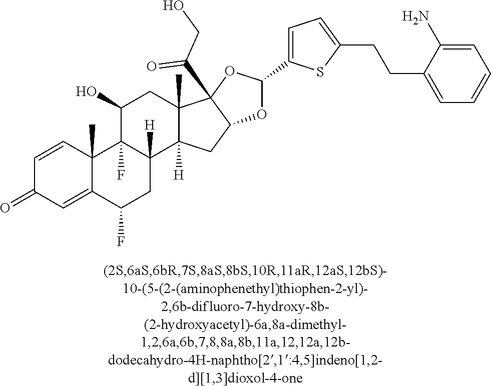 |
| 80 | 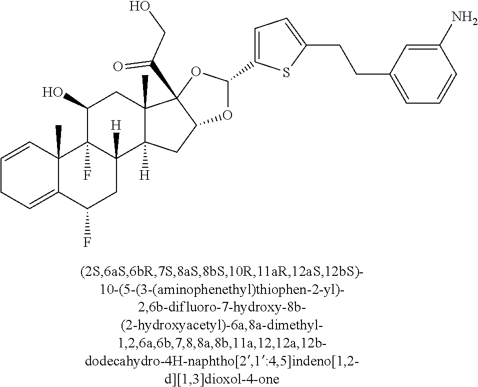 |
| 81 | 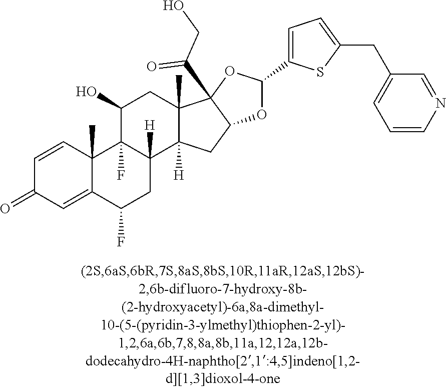 |
| 82 | 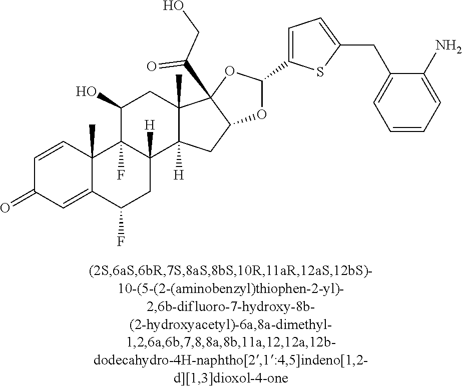 |
| 83 | 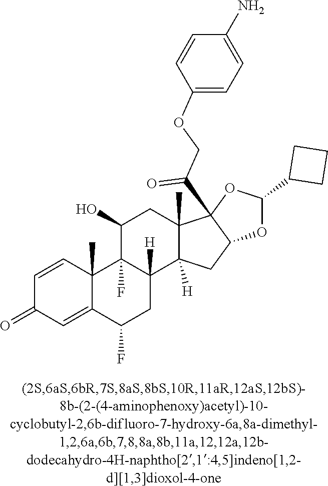 |

In some embodiments of the compound of the present disclosure or pharmaceutically acceptable salt thereof, the compound has a structure selected from Table 2.

TABLE 2
Compounds

| Cmpd. | Structure/Name |
|---|---|
| 84 | 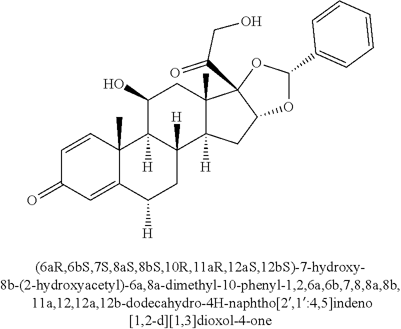 |
| 85 | 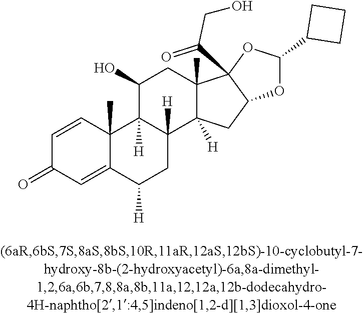 |
| 86a | 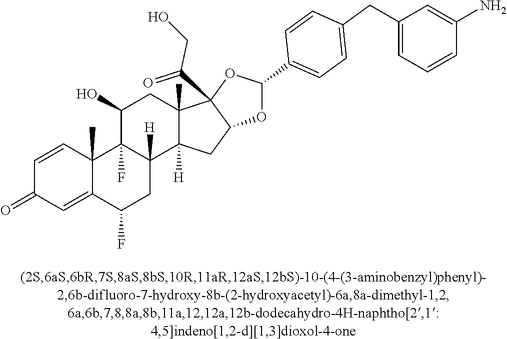 |
| 86b | 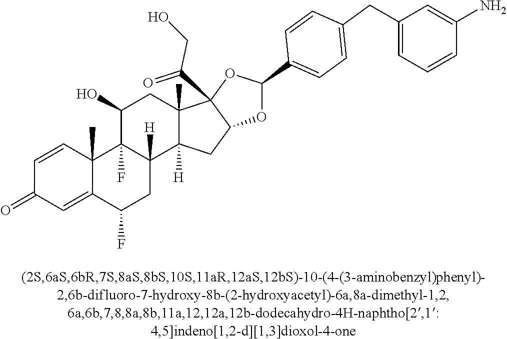 |
| 87 | 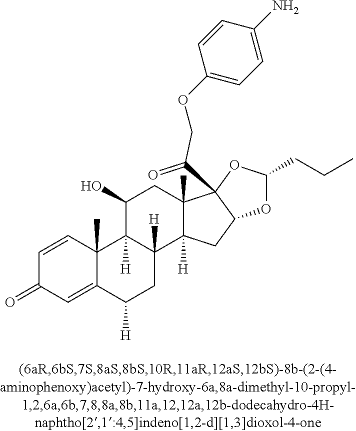 |
| 88 | 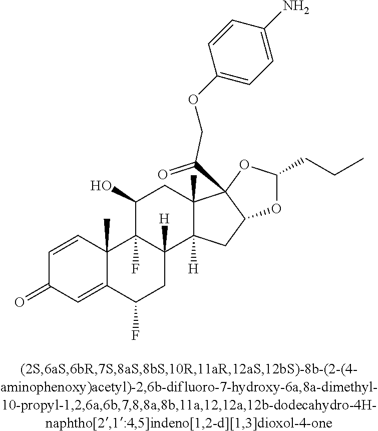 |
| 89a | 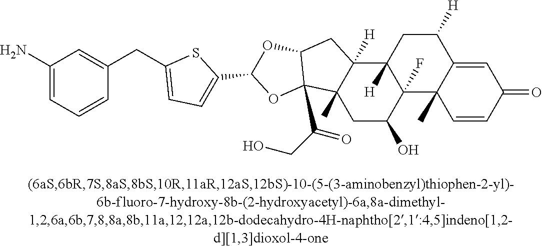 |
| 89b | 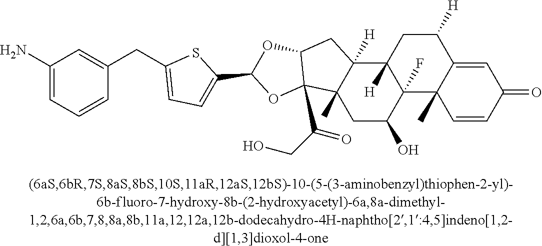 |
| 90a | 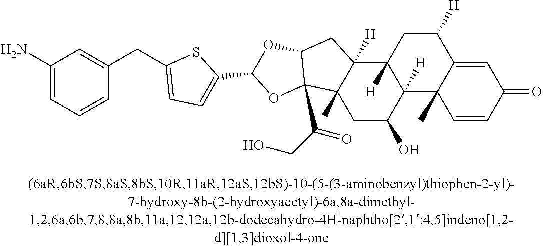 |
| 90b | 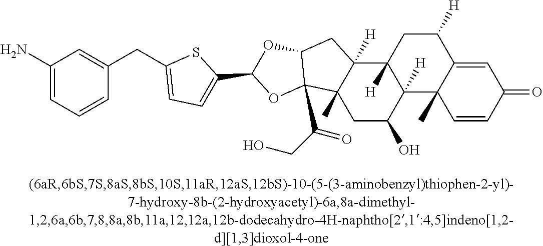 |
| 91a | 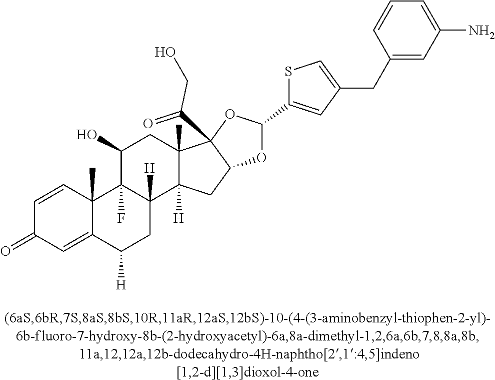 |
| 91b | 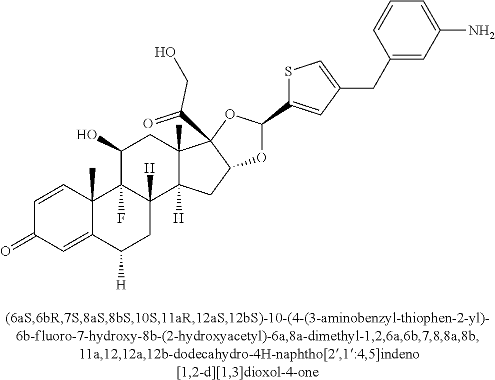 |
| 92a | 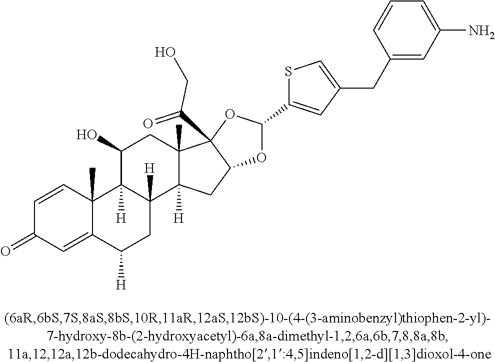 |
| 92b | 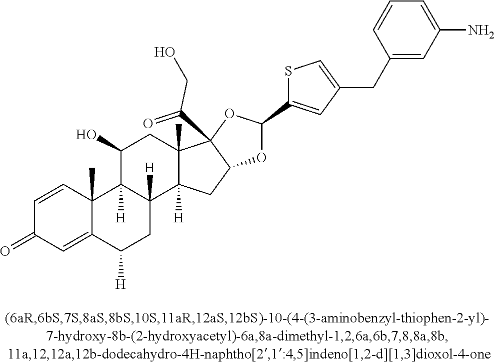 |

Characterization

Compound 30a

¹H NMR (400 MHz, DMSO-d₆) δ=7.63-7.59 (m, 1H), 7.27 (d, J=10.0 Hz, 1H), 6.96 (d, J=5.2 Hz, 1H), 6.33-6.27 (m, 1H), 6.13 (s, 1H), 5.93 (s, 1H), 5.75-5.56 (m, 1H), 5.53 (s, 1H), 4.95 (s, 1H), 4.50 (d, J=19.2 Hz, 1H), 4.28-4.13 (m, 2H), 2.74-2.56 (m, 1H), 2.36-2.28 (m, 1H), 2.27-2.17 (m, 1H), 2.09-1.99 (m, 1H), 1.74-1.64 (m, 3H), 1.55-1.41 (m, 4H), 0.85 (s, 3H). MS (ESI) m/z 525.0 [M+H]⁺

Compound 30B

¹H NMR (400 MHz, DMSO-d₆) δ=7.63-7.59 (m, 1H), 7.27 (d, J=10.0 Hz, 1H), 6.96 (d, J=5.2 Hz, 1H), 6.48 (s, 1H), 6.33-6.27 (m, 1H), 6.13 (s, 1H), 5.75-5.56 (m, 3H), 5.29 (d, J=6.0 Hz, 1H), 4.40-4.05 (m, 3H), 2.74-2.56 (m, 1H), 2.36-2.28 (m, 1H), 2.24-2.05 (m, 2H), 1.99-1.61 (m, 5H), 1.48 (s, 3H), 0.86 (s, 3H). MS (ESI) m/z 525.2 [M+H]⁺

Compound 52a

¹H NMR (400 MHz, DMSO-d₆) δ=7.64-7.57 (m, 1H), 7.29 (d, J 9.6 Hz, 1H), 6.96 (d, J=5.2 Hz, 1H), 6.27-6.20 (m, 1H), 6.04 (s, 1H), 5.92 (s, 1H), 5.45 (s, 1H), 4.97-4.90 (m, 1H), 4.50 (d, J=19.6 Hz, 1H), 4.22-4.14 (m, 3H), 2.69-2.54 (m, 2H), 2.48-2.42 (m, 1H), 2.40-2.31 (m, 1H), 2.22-2.10 (m, 1H), 2.07-1.99 (m, 1H), 1.90-1.80 (m, 1H), 1.70-1.60 (m, 3H), 1.49 (s, 3H), 1.44-1.33 (m, 1H), 0.86 (s, 3H). MS (ESI) m/z 507.2 [M+H]⁺

Compound 52b

¹H NMR (400 MHz, DMSO-d₆) δ=7.63-7.58 (m, 1H), 7.29 (d, J=10.0 Hz, 1H), 6.97 (d, J=5.6 Hz, 1H), 6.45 (s, 1H), 6.27-6.21 (m, 1H), 6.03 (s, 1H), 5.47 (s, 1H), 5.28 (d, J=6.8 Hz, 1H), 4.31 (d, J=19.2 Hz, 1H), 4.22-4.15 (m, 1H), 4.07 (d, J=19.6 Hz, 1H), 2.69-2.58 (m, 1H), 2.48-2.30 (m, 2H), 2.07-1.96 (m, 2H), 1.87-1.76 (m, 2H), 1.74-1.65 (m, 2H), 1.56-1.42 (m, 5H), 0.87 (s, 3H). MS (ESI) m/z 507.2 [M+H]⁺

Compound 53a

¹H NMR (400 MHz, DMSO-d₆) δ=7.65-7.51 (m, 1H), 7.31 (d, J=9.6 Hz, 1H), 6.96 (d, J=4.8 Hz, 1H), 6.17 (d, J=10 Hz, 1H), 5.92 (d, J=19.6 Hz, 2H), 4.92 (s, 1H), 4.87-4.75 (m, 1H), 4.49 (d, J=18.8 Hz, 1H), 4.32 (s, 1H), 4.16 (d, J=19.6 Hz, 1H), 2.62-2.53 (m, 1H), 2.39-2.28 (m, 1H), 2.21-1.98 (m, 3H), 1.75 (s, 2H), 1.72-1.61 (m, 3H), 1.39 (s, 3H), 1.05-0.93 (m, 1H), 0.90 (s, 1H), 0.85 (s, 3H). MS (ESI) m/z 489.0 [M+H]⁺

Compound 53b

¹H NMR (400 MHz, DMSO-d₆) δ=7.63-7.57 (m, 1H), 7.31 (d, J=10.0 Hz, 1H), 6.96 (d, J 5.2 Hz, 1H), 6.44 (s, 1H), 6.21-6.12 (m, 1H), 5.93 (s, 1H), 5.25 (d, J=6.4 Hz, 1H), 4.90-4.68 (m, 1H), 4.38-4.27 (m, 3H), 4.06 (d, J=19.2 Hz, 1H), 2.59-2.52 (m, 1H), 2.34-2.26 (m, 1H), 2.10-1.94 (m, 2H), 1.88-1.62 (m, 6H), 1.38 (s, 3H), 1.27-1.13 (m, 1H), 1.10-1.03 (m, 1H), 0.86 (s, 3H). MS (ESI) m/z 489.2 [M+H]⁺

Compound 61a

¹H NMR (400 MHz, DMSO-d₆) δ=7.27 (dd, J=0.8, 10.0 Hz, 1H), 7.19 (t, J=8.0 Hz, 1H), 7.09 (d, J=3.2 Hz, 1H), 6.92 (d, J=7.6 Hz, 1H), 6.88-6.82 (m, 2H), 6.75 (d, J=3.6 Hz, 1H), 6.30 (dd, J=1.6, 10.0 Hz, 1H), 6.14 (s, 1H), 5.75 (s, 1H), 5.73-5.50 (m, 2H), 4.91 (t, J=2.4 Hz, 1H), 4.49 (d, J=19.2 Hz, 1H), 4.23-4.14 (m, 2H), 4.07 (s, 2H), 2.73-2.55 (m, 1H), 2.34-2.26 (m, 1H), 2.20 (q, J=10.0 Hz, 1H), 1.99 (d, J=13.6 Hz, 1H), 1.72-1.57 (m, 3H), 1.49 (s, 3H), 1.47-1.39 (m, 1H), 0.84 (s, 3H). MS (ESI) m/z 612.0 [M+H]⁺

Compound 61b

¹H NMR (400 MHz, DMSO-d₆) δ=7.26 (d, J=11.2 Hz, 1H), 7.21-7.15 (m, 1H), 7.04 (d, J=3.6 Hz, 1H), 6.88-6.73 (m, 4H), 6.34 (s, 1H), 6.30 (dd, J=1.6, 10.0 Hz, 1H), 6.12 (s, 1H), 5.76-5.55 (m, 1H), 5.53 (d, J=3.6 Hz, 1H), 5.26 (d, J=7.2 Hz, 1H), 4.37 (d, J 12.8 Hz, 1H), 4.23-4.15 (m, 1H), 4.12-4.02 (m, 3H), 2.30-1.98 (m, 4H), 1.91-1.78 (m, 1H), 1.75-1.59 (m, 3H), 1.49 (s, 3H), 0.87 (s, 3H). MS (ESI) m/z 612.2 [M+H]⁺

Compound 62a

¹H NMR (400 MHz, DMSO-d₆) δ=7.29-7.23 (m, 2H), 7.20 (br t, J=7.7 Hz, 1H), 7.05 (d, J=1.3 Hz, 1H), 6.91 (br s, 1H), 6.87-6.78 (m, 2H), 6.30 (dd, J=1.9, 10.2 Hz, 1H), 6.13 (s, 1H), 5.81 (s, 1H), 5.75-5.56 (m, 1H), 5.52 (br d, J=1.8 Hz, 1H), 4.91 (t, J=2.6 Hz, 1H), 4.49 (d, J=19.4 Hz, 1H), 4.26-4.11 (m, 2H), 3.82 (s, 2H), 2.75-2.56 (m, 1H), 2.38-2.17 (m, 3H), 2.07-1.95 (m, 1H), 1.70-1.60 (m, 3H), 1.49 (s, 3H), 1.48-1.40 (m, 1H), 0.84 (s, 3H). MS (ESI) m/z 612.1 [M+H]⁺

Compound 62b

¹H NMR (400 MHz, MeOD) δ=7.48-7.39 (m, 1H), 7.32 (br d, J=10.0 Hz, 2H), 7.21-7.11 (m, 2H), 6.98 (br s, 1H), 6.87 (s, 1H), 6.40 (s, 1H), 6.37-6.27 (m, 2H), 5.65-5.45 (m, 1H), 5.33 (d, J=6.4 Hz, 1H), 4.39 (d, J=19.3 Hz, 1H), 4.29 (br d, J=9.5 Hz, 1H), 4.16 (d, J=19.3 Hz, 1H), 3.98 (s, 2H), 2.76-2.55 (m, 1H), 2.43-2.29 (m, 1H), 2.19-2.13 (m, 2H), 1.95-1.75 (m, 2H), 1.73-1.63 (m, 2H), 1.57 (s, 3H), 0.97 (s, 3H). MS (ESI) m/z 612.1 [M+H]⁺

Compound 86a

¹H NMR (400 MHz, DMSO-d₆) δ=7.36 (d, J=8.0 Hz, 2H), 7.29-7.22 (m, 4H), 7.02 (d, J=7.6 Hz, 1H), 6.95-6.85 (m, 2H), 6.30 (dd, J=1.6, 10.0 Hz, 1H), 6.13 (s, 1H), 5.71-5.60 (m, 2H), 5.46 (s, 1H), 4.95 (d, J=4.4 Hz, 1H), 4.51 (d, J=19.2 Hz, 1H), 4.27-4.13 (m, 2H), 3.91 (s, 2H), 2.75-2.56 (m, 1H), 2.35-2.17 (m, 2H), 2.02 (d, J=13.2 Hz, 1H), 1.76-1.63 (m, 3H), 1.54-1.45 (m, 4H), 0.86 (s, 3H). MS (ESI) m/z 606.3 [M+H]⁺

Compound 86b

¹H NMR (400 MHz, DMSO-d₆) δ=7.26 (d, J=10.4 Hz, 1H), 7.23-7.16 (m, 5H), 6.89 (d, J=7.6 Hz, 1H), 6.84-6.75 (m, 2H), 6.30 (dd, J=2.0, 10.2 Hz, 1H), 6.14-6.07 (m, 2H), 5.74-5.55 (m, 1H), 5.54-5.49 (m, 1H), 5.33 (d, J=7.2 Hz, 1H), 4.26 (d, J=19.2 Hz, 1H), 4.20-4.15 (m, 1H), 4.03 (d, J=19.2 Hz, 1H), 3.89 (s, 2H), 2.68-2.56 (m, 1H), 2.35-2.24 (m, 2H), 2.20-2.10 (m, 2H), 2.09-2.04 (m, 1H), 1.93-1.80 (m, 1H), 1.77-1.60 (m, 3H), 1.49 (s, 3H), 0.88 (s, 3H). MS (ESI) m/z 606.3 [M+H]⁺

Compound 89a

¹H NMR (400 MHz, DMSO-d₆) δ=7.29 (d, J=10.0 Hz, 1H), 7.23-7.16 (m, 1H), 7.09 (d, J=3.6 Hz, 1H), 6.92-6.81 (m, 3H), 6.75 (d, J=3.6 Hz, 1H), 6.24 (dd, J=1.6, 10.0 Hz, 1H), 6.05 (s, 1H), 5.74 (s, 1H), 5.43 (s, 1H), 4.89 (d, J=3.6 Hz, 1H), 4.49 (d, J=19.2 Hz, 1H), 4.17 (d, J=19.2 Hz, 2H), 4.07 (s, 2H), 2.70-2.59 (m, 1H), 2.40-2.31 (m, 1H), 2.18-2.08 (m, 1H), 2.07-1.99 (m, 1H), 1.87-1.78 (m, 1H), 1.71-1.58 (m, 3H), 1.50 (s, 3H), 1.38-1.33 (m, 1H), 0.85 (s, 3H). MS (ESI) m/z 594.2 [M+H]⁺

Compound 89b

¹H NMR (400 MHz, DMSO-d₆) δ=7.29 (d, J=10.0 Hz, 1H), 7.19 (t, J=7.6 Hz, 1H), 7.05 (d, J=3.6 Hz, 1H), 6.90-6.75 (m, 4H), 6.31 (s, 1H), 6.24 (dd, J=1.6, 10.0 Hz, 1H), 6.04 (s, 1H), 5.45 (d, J=2.4 Hz, 1H), 5.24 (d, J=6.8 Hz, 1H), 4.36 (d, J=19.2 Hz, 1H), 4.18 (d, J=10.0 Hz, 1H), 4.12-4.04 (m, 3H), 2.71-2.60 (m, 1H), 2.39-2.32 (m, 2H), 2.07-1.97 (m, 2H), 1.89-1.77 (m, 2H), 1.71-1.63 (m, 2H), 1.50 (s, 3H), 0.88 (s, 3H). MS (ESI) m/z 594.3 [M+H]⁺

Compound 90a

¹H NMR (400 MHz, DMSO-d₆) δ=7.32 (d, J=10.0 Hz, 1H), 7.26-7.19 (m, 1H), 7.09 (d, J=3.6 Hz, 1H), 6.97 (d, J=7.6 Hz, 1H), 6.93-6.87 (m, 2H), 6.75 (d, J=3.6 Hz, 1H), 6.19 (dd, J=1.6, 10.0 Hz, 1H), 5.97 (s, 1H), 5.73 (s, 1H), 4.88 (d, J=4.4 Hz, 1H), 4.86-4.76 (m, 1H), 4.49 (d, J 19.2 Hz, 1H), 4.29 (d, J 2.4 Hz, 1H), 4.16 (d, J=19.6 Hz, 1H), 4.09 (s, 2H), 2.58-2.52 (m, 1H), 2.35-2.30 (m, 1H), 2.18-1.97 (m, 2H), 1.79-1.54 (m, 5H), 1.39 (s, 3H), 1.01-0.86 (m, 2H), 0.84 (s, 3H). MS (ESI) m/z 576.2 [M+H]⁺

Compound 90b

¹H NMR (400 MHz, DMSO-d₆) δ=7.31 (d, J=10.0 Hz, 1H), 7.22 (t, J=7.6 Hz, 1H), 7.01 (d, J=3.2 Hz, 1H), 6.92 (d, J=7.6 Hz, 1H), 6.89-6.82 (m, 2H), 6.77 (d, J=3.2 Hz, 1H), 6.30 (s, 1H), 6.17 (d, J=10.0 Hz, 1H), 5.94 (s, 1H), 5.20 (d, J=6.8 Hz, 1H), 4.80 (s, 1H), 4.36 (d, J=19.2 Hz, 1H), 4.29 (s, 1H), 4.10-3.99 (m, 3H), 2.50-2.40 (m, 1H), 2.12-1.95 (m, 2H), 1.87-1.59 (m, 5H), 1.38 (s, 3H), 1.27-1.09 (m, 1H), 1.05-1.00 (m, 1H), 0.86 (s, 3H). MS (ESI) m/z 576.3 [M+H]⁺

Compound 91a

¹H NMR (400 MHz, DMSO-d₆) δ=7.29-7.23 (m, 2H), 7.20 (br t, J=7.6 Hz, 1H), 7.05 (s, 1H), 6.91 (br s, 1H), 6.87-6.78 (m, 2H), 6.30 (dd, J=2.0, 10.2 Hz, 1H), 6.13 (s, 1H), 5.81 (s, 1H), 5.42 (br d, J=1.8 Hz, 1H), 4.91 (t, J=2.6 Hz, 1H), 4.49 (d, J=19.4 Hz, 1H), 4.26-4.11 (m, 2H), 3.82 (s, 2H), 2.75-2.56 (m, 1H), 2.38-2.17 (m, 3H), 2.07-1.95 (m, 1H), 1.69-1.60 (m, 3H), 1.49 (s, 3H), 1.48-1.40 (m, 1H), 0.84 (s, 3H). MS (ESI) m/z 594.1 [M+H]⁺

Compound 91b

¹H NMR (400 MHz, MeOD) δ=7.44-7.37 (m, 2H), 7.29 (d, J=7.8 Hz, 1H), 7.18-7.11 (m, 2H), 6.96 (s, 1H), 6.87 (s, 1H), 6.37 (s, 1H), 6.30 (d, J=10.4 Hz 1H), 6.10 (s, 1H), 5.32 (d, J=6.4 Hz, 1H), 4.38 (d, J=19.2 Hz, 1H), 4.37-4.22 (m, 1H), 4.15 (d, J=19.2 Hz, 1H), 3.99 (s, 2H), 2.81-2.70 (m, 1H), 2.68-2.51 (m, 1H), 2.49-2.40 (m, 1H), 2.28-2.10 (m, 2H), 1.99-1.52 (m, 5H), 1.63 (s, 3H), 0.97 (s, 3H). MS (ESI) m/z 594.2 [M+H]⁺

Compound 92a

¹H NMR (400 MHz, DMSO-d₆) δ=7.31 (d, J=10.0 Hz, 1H), 7.22 (s, 1H), 7.21-7.16 (m, 1H), 7.04 (d, J=1.4 Hz, 1H), 6.90 (br d, J=4.0 Hz, 1H), 6.87-6.79 (m, 2H), 6.17 (dd, J=2.0, 10.0 Hz, 1H), 5.95 (s, 1H), 5.78 (s, 1H), 4.88 (d, J=4.8 Hz, 1H), 4.81 (br s, 1H), 4.48 (d, J=19.2 Hz, 1H), 4.30 (br d, J=2.8 Hz, 1H), 4.15 (d, J=19.2 Hz, 1H), 3.82 (s, 2H), 2.60-2.52 (m, 1H), 2.37-2.28 (m, 1H), 2.18-2.01 (m, 2H), 1.76-1.71 (m, 2H), 1.70-1.65 (m, 2H), 1.64-1.59 (m, 1H), 1.39 (s, 3H), 1.05-0.86 (m, 2H), 0.85 (s, 3H). MS (ESI) m/z 576.1 [M+H]⁺

Compound 92b

¹H NMR (400 MHz, MeOD) δ=7.51-7.40 (m, 2H), 7.35 (br d, J=7.2 Hz, 1H), 7.24-7.13 (m, 2H), 6.99 (br s, 1H), 6.85 (s, 1H), 6.38 (s, 1H), 6.27 (dd, J=1.6, 10.0 Hz, 1H), 6.04 (s, 1H), 5.31 (t, J=3.2 Hz, 1H), 4.44 (br d, J=2.8 Hz, 1H), 4.41-4.34 (m, 1H), 4.17 (d, J=19.2 Hz, 1H), 4.00 (s, 2H), 2.69-2.64 (m, 1H), 2.44-2.39 (m, 1H), 2.31-2.11 (m, 2H), 2.04-1.98 (m, 1H), 1.89-1.80 (m, 2H), 1.79-1.64 (m, 2H), 1.51 (s, 3H), 1.27-1.16 (m, 1H), 1.13-1.09 (m, 1H), 0.99 (s, 3H). MS (ESI) m/z 576.2 [M+H]⁺

### IV. Conjugates

In some embodiments, a conjugate of the present disclosure comprises a compound of the present disclosure, a binding protein as described herein, and a linker covalently attaching the compound to the binding protein.

In some embodiments, conjugates of this disclosure will have an average ratio of the GR agonist to binding protein (referred to herein as a drug-to-antibody ratio, or DAR) that ranges from 1 to about 20, from 1 to about 10, from 1 to about 8, from 1 to about 6, from 1 to about 5, from 1 to about 3, from 2 to about 8, from 2 to about 6, from 2 to about 5, from 2 to about 4, from about 3 to about 8, from about 3 to about 6, or from about 3 to about 5, wherein the drug is a GR agonist of any one of the formulae of this disclosure. In certain embodiments, the average drug-to-antibody ratio (DAR) of a conjugate of this disclosure ranges from 1 to about 8, or 2 to about 6, or about 3 to about 5, or about 4. In some embodiments, the average ratio of the GR agonist to binding protein of conjugates in a pharmaceutical formulation may range from 1 to 20, from 1 to 10, from 1 to 8, from 1 to 6, from 1 to 5, from 1 to 3, from 2 to 8, from 2 to 6, from 2 to 5, from 2 to 4, from 3 to 8, from 3 to 6, or from 3 to 5, wherein the drug is a GR agonist of any one of the formulae of this disclosure. In some embodiments, the average DAR of a conjugate of this disclosure is 2 or about 2, 3 or about 3, 4 or about 4, 5 or about 5, 6 or about 6, 7 or about 7, or 8 or about 8.

A. Antibodies and Fusion Proteins

In some embodiments of this disclosure, a binding protein or conjugate thereof comprises an antibody (e.g., a monoclonal antibody) or a fusion protein comprising a binding domain that specifically binds to a target of interest. In some embodiments, an anti-target antibody or antigen binding fragment thereof or a fusion protein is conjugated to a compound of the present disclosure, thus forming a conjugate or binding protein conjugate.

In some embodiments, a binding protein conjugate of this disclosure comprises an anti-target fusion protein from naturally occurring or non-naturally occurring source sequences. In some embodiments, an antibody of the disclosure is recombinant.

An antibody of the disclosure may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody can be chimeric or humanized. In some embodiments, an antibody of the disclosure is chimeric. Chimeric and humanized forms of non-human (e.g., murine) antibodies can be intact (full length) chimeric immunoglobulins, immunoglobulin chains or antigen binding fragments thereof (such as Fv, Fab, Fab′, F(ab′)₂ or other target-binding subdomains of antibodies), which can contain sequences derived from non-human immunoglobulin. In some embodiments, an antibody of the disclosure is humanized. In general, a humanized antibody can comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc) or an Fc domain, such as a human immunoglobulin sequence.

An antibody of the disclosure can be a human antibody. As used herein, a “human antibody” can include antibodies having, for example, the amino acid sequence of a human immunoglobulin and include antibodies isolated from human immunoglobulin libraries or from animals transgenic for one or more human immunoglobulins and that typically do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, is used to guide the selection of a completely human antibody recognizing the same epitope.

An antibody can be any class, e.g., IgA, IgD, IgE, IgG, and IgM. Certain classes can be further divided into isotypes, e.g., IgG1, IgG2, IgG3, IgG4, IgA1, and IgA2. The heavy-chain constant regions that correspond to the different classes of immunoglobulins can be α, δ, ε, γ, and μ, respectively. In certain embodiments, an antibody of this disclosure comprises a human IgG1, human IgG2, human IgG3, or human IgG4 heavy chain constant region. The light chains can be either kappa (or κ) or lambda (or λ).

An antibody of the disclosure can be a bispecific antibody or a dual variable domain antibody (DVD). Bispecific and DVD antibodies are monoclonal, often human or humanized, antibodies that have binding specificities for at least two different antigens. In various embodiments, at least one binding domain of a bispecific antibody specifically binds to a target provided in this disclosure. Similarly, a fusion protein of this disclosure can be bispecific and have two binding domains that bind to two different targets, such as BAFF and APRIL, CD28 and ICOS, or BAFF and ICOSL. In some embodiments, one of the two binding domains may be an antigen-binding domain from or derived from an antibody.

In some embodiments, an antibody or fusion protein of this disclosure comprises an antigen binding domain and an Fc domain. In various embodiments, an antibody comprises two light chain polypeptides (light chains) and two heavy chain polypeptides (heavy chains), held together covalently by disulfide linkages. The heavy chain typically comprises a heavy chain variable region (VH) and a heavy chain constant region. The heavy chain constant region comprises three domains, CH1, CH2, and CH3. An Fc domain is located within the heavy chain CH2 and CH3 domains. Non-limiting exemplary heavy chain constant regions include human IgG1, human IgG2, human IgG3, and human IgG4 constant regions. In some embodiments, an antibody provided herein comprises an IgG1 heavy chain constant region. In further embodiments, an antibody provided herein comprises an IgG1 heavy chain constant region comprising one or more substitutions that reduce or eliminate effector function. In some embodiments, an antibody provided herein comprises an IgG1 heavy chain constant region (e.g., human IgG1 constant region) comprising L117A, L118A, G120A, and/or K205A substitutions.

In some embodiments, an antibody provided herein comprises an IgG1 constant region comprising P329G, L234A, L235A, G237A, and/or K322A substitutions. Non-limiting exemplary human IgG1 constant region and human IgG1 null constant region amino acid sequences are shown in SEQ ID NOS:181 and 182. The light chain typically comprises a light chain variable region (VL) and a light chain constant region. Non-limiting exemplary light chain constant regions include kappa and lambda constant regions. A non-limiting exemplary human kappa constant region amino acid sequence is shown in SEQ ID NO:183.

The antigen-recognition regions of antibody variable domains typically comprise six complementarity determining regions (CDRs), or hypervariable regions, that lie within the framework of the heavy chain variable region and light chain variable region at the N-terminal ends of the two heavy and two light chains. In some embodiments an antigen binding domain comprises a light chain complementary determining region 1 (LCDR1), a light chain complementary determining region 2 (LCDR2), a light chain complementary determining region 3 (LCDR3), a heavy chain complementary determining region 1 (HCDR1), a heavy chain complementary determining region 2 (HCDR2), and a heavy chain complementary determining region 3 (HCDR3). In some embodiments, an antibody may be a heavy-chain only antibody, in which case the antigen binding domain comprises HCDR1, HCDR2, and HCDR3, and the antibody lacks a light chain.

Exemplary CDR sequences of antibodies disclosed herein, such as anti-BAFF antibodies, anti-LPAM-1 antibodies, anti-CD40 antibodies, anti-CD86 antibodies, anti-ICOS antibodies, anti-ICOSL antibodies, anti-CD28 antibodies, anti-CD80 antibodies, and anti-Integrin β7 antibodies, may be determined by one or more methods, including Kabat, Chothia, AbM, Contact, IMGT and AHo. Unless otherwise specified herein, CDR sequences are determined according to the Kabat method. References to variable region or CDR numbering as in Kabat, amino acid position numbering as in Kabat, or CDR sequences determined according to the Kabat method, and variations thereof, refer to the numbering system used for heavy chain variable regions or light chain variable regions of the compilation of antibodies in Kabat et al. ((1991) Sequences of Proteins of Immunological Interest, Fifth Edition, U.S. Department of Health and Human Services, NIH Publication No. 91-3242). Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra).

The “EU numbering system” or “EU index” is generally used when referring to a residue in an immunoglobulin heavy chain constant region (e.g., the EU index reported in Kabat et al., supra). The “EU index, as in Kabat,” refers to the residue numbering of the human IgG 1 EU antibody.

Other numbering systems have been described, for example, by AbM (Oxford Molecular's AbM antibody modeling software (see, e.g., Antibody Engineering Vol. 2 (Kontermann and Dithel eds., 2d ed. 2010)), Chothia (see, Chothia and Lesk, 1987, J. Mol. Biol. 196:901-17), Contact, IMGT (ImMunoGeneTics (IMGT) Information System® (see, Lafranc et al., 2003, Dev. Comp. Immunol. 27(1):55-77)), and AHon (see, Honegger and Plückthun, 2001, J. Mol. Biol. 309: 657-70) and are well understood by a person of ordinary skill in the art.

B. Constant Domains and Fc Region Portions

The constant domains of an antibody provide the general framework of an antibody, and may not be involved directly in binding to an antigen, but can be involved in various effector functions, such as participation of the antibody in antibody-dependent cellular cytotoxicity (ADCC), ADCP (antibody-dependent cellular phagocytosis), CDC (complement-dependent cytotoxicity) and complement fixation, binding to Fc receptors (e.g., CD16, CD32, FcRn), greater in vivo half-life relative to a polypeptide lacking an Fc region, protein A binding, and perhaps even placental transfer (see Capon et al., Nature 337:525, 1989).

As used herein, an “Fc region constant domain portion,” or “Fc region portion” refers to the heavy chain constant region segment of the Fc fragment (the “fragment crystallizable” region or Fc region) from an antibody, which can in include one or more constant domains, such as CH2, CH3, CH4, or any combination thereof. An “Fc domain” as used herein refers to a domain from an Fc region portion of an antibody that can specifically bind to an Fc receptor, such as an Fcγ receptor or an FcRn receptor. In certain embodiments, an Fc region portion includes the CH2 and CH3 domains of an IgG, IgA, or IgD antibody and any combination thereof, or the CH3 and CH4 domains of an IgM or IgE antibody and any combination thereof.

An Fc region or domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), trogocytosis, trogoptosis, and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YXXL sequence (where X=any amino acid) flanking seven variable residues. The SRC and SYK kinases may connect the transduced signals with common activation pathways.

In some embodiments, an Fc region portion or domain thereof can exhibit reduced binding affinity to one or more Fc receptors, such as Fcγ receptors, FcRn receptors, or Fcγ and FcRn receptors. In some embodiments, an Fc region portion comprises an Fc null domain. As used herein, an “Fc null domain” refers to a domain that exhibits weak to no binding to any of the Fcγ receptors. In some embodiments, an Fc null domain exhibits a reduction in binding affinity (e.g., increase in Kd) to Fcγ receptors of at least about 1000-fold.

The Fc region or domain may have one or more, two or more, three or more, or four or more, or up to five amino acid substitutions that decrease binding of the Fc region portion or domain thereof to an Fc receptor. In some embodiments, an Fc region portion or domain thereof exhibits decreased binding to FcγRI (CD64), FcγRIIA (CD32), FcγRIIIA (CD16a), FcγRIIIB (CD16b), or any combination thereof. In some embodiments, the Fc region portion or domain thereof is an IgG1 and the one or more substitutions in the Fc region or domain comprise any one or combination of IgG1 heavy chain mutations corresponding to P329G, E233P, L234V, L234A, L235A, L235E, AG236, G237A, E318A, K320A, K322A, A327G, A330S, or P331S according to the EU index of Kabat numbering.

In some embodiments, an Fc region portion or domain thereof can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (referred to as IgG1VLPLL) according to the EU index of Kabat numbering. A modification can comprise a substitution at more than one amino acid residues, such as at 2 different amino acid residues including S239D/1332E (IgG1DE) according to the EU index of Kabat numbering. A modification can comprise a substitution at more than one amino acid residue, such as at 3 different amino acid residues including S298A/E333A/K334A (referred to as IgG1AAA) according to the EU index of Kabat numbering. In certain embodiments of this disclosure, IgG1 constant regions comprise or consist of an amino acid sequence of any one of SEQ ID NOS:181-183. In further embodiments, an antibody of this disclosure comprises a mouse IgG2a heavy chain constant region.

An antibody or an Fc region portion or domain thereof may be modified to acquire or improve at least one constant region-mediated biological effector function relative to an unmodified antibody or Fc domain, e.g., to enhance FcγR interactions. In some embodiments, a modification can increase CD32b binding (and support, for example, transdelivery in a PBMC assay) comprises a substitution at S267L and E329F (IgG1LF, also known as SELF double mutant) according to the EU index of Kabat numbering. In certain embodiments, an antibody of this disclosure may comprise an Fc domain, or a fusion protein of this disclosure may comprise an Fc region portion, that binds to FcγRIIA, FcγRIIB or FcγRIIIA with greater affinity than the corresponding wild-type Fc domain.

In some embodiments, an Fc region portion or domain thereof found in an antibody or fusion protein of this disclosure is capable of mediating one or more effector functions, lacks one or more or all such activities, or has one or more of the effector activities increased by way of, for example, one or more mutations as compared to an unmodified Fc region portion or domain thereof.

In some embodiments, an IgG Fc domain comprises at least one amino acid substitution that reduces its binding affinity to FcγR1, as compared to a wild-type or reference IgG Fc domain. Such a modification can comprise a substitution at F241, such as F241A, at F243, such as F243A, at V264, such as V264A, or at D265, such as D265A, each according to the EU index of Kabat.

In some embodiments, an IgG Fc domain comprises at least one amino acid substitution that increases its binding affinity to FcγR1, as compared to a wild-type or reference IgG Fc domain. Such a modification can comprise a substitution at A327 and P329, such as A327Q/P329A, according to the EU index of Kabat.

In some embodiments, the modification comprises substitution of one or more amino acids that reduce binding affinity of an IgG Fc domain to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitutions of one or more amino acids that increase binding affinity of an IgG Fc domain to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat.

A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitutions of one or more amino acids that decrease binding affinity of an IgG Fc domain to FcγRII receptor but do not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitutions of one or more amino acids that decrease binding affinity of an IgG Fc domain to FcγRII receptor and increase the binding affinity to FcγRIIIA receptor. A modification can be a substitution of S298, such as S298A, according to the EU index of Kabat. A modification can be substitution of S239, I332 and A330, such as S239D/I332E/A330L. A modification can be substitution of S239 and I332, such as S239D/I332E.

In some embodiments, the modification comprises substitution of one or more amino acids that decreases binding affinity of an IgG Fc domain to FcγRIIIA receptor. A modification can be substitutions of F241 and F243, such as F241 S/F243 S or F241I/F243I, according to the EU index of Kabat.

In some embodiments, the modification comprises substitutions of one or more amino acids that decrease binding affinity of an IgG Fc domain to FcγRIIIA receptor and do not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitutions of one or more amino acids that increase binding affinity of an IgG Fc domain to FcγRIIIA receptor and do not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitutions of S239 and I332, such as S239D/I332E.

In some embodiments, the modification comprises substitutions of one or more amino acids that increase binding affinity of an IgG Fc domain to FcγRIIIA receptor. A modification can be substitutions of L235, F243, R292, Y300 and P396, such as L235V/F243L/R292P/Y300L/P396L (IgG1VLPLL) according to the EU index of Kabat. A modification can be substitutions of S298, E333 and K334, such as S298A/E333A/K334A, according to the EU index of Kabat. A modification can be a substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the substitutions of which are incorporated by reference herein).

In some embodiments, an IgG Fc domain comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be substitutions of one or more amino acids selected from P257L, P257N, P257I, V279E, V279Q, V279Y, A281 S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

In some embodiments, an antibody is a human IgG2 antibody, including an IgG2 Fc region. In some embodiments, the heavy chain of the human IgG2 antibody can be mutated at cysteines at positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

C. Targets

As used herein, “target” refers to a molecule of interest to allow specific delivery of a conjugate of this disclosure to the location or tissue where the target is located. Exemplary targets of this disclosure include one or more targets selected from B-cell-activating factor (BAFF), BAFF receptor (BAFF-R), A proliferation-inducing ligand (APRIL), transmembrane activator and CAML interactor (TACI), Peyer patches-specific homing receptor (LPAM-1), B-cell maturation antigen (BCMA), CD40, CD40 Ligand (CD40L), T-lymphocyte activation antigen CD86 (CD86), cytotoxic T-lymphocyte protein 4 (CTLA4), tyrosine kinase-type cell surface receptor HER2 (HER2), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), and tumor necrosis factor receptor 2 (TNF-R2). In some embodiments, the target is selected from the group consisting of cluster of differentiation 40 (CD40, tumor necrosis factor receptor superfamily 5 (TNFSF5)), CD40 Ligand (CD40L, CD154), T-lymphocyte activation antigen CD86 (CD86), cytotoxic T-lymphocyte protein 4 (CTLA4), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), tumor necrosis factor receptor 2 (TNF-R2), killer cell lectin-like receptor G1 (KLRG1), B-cell-activating factor (BAFF), BAFF Receptor (BAFFR), transmembrane activator and CAML interactor (TACI), Peyer patches-specific homing receptor (LPAM-1), B-cell maturation antigen (BCMA), and a proliferation-inducing ligand (APRIL). In some embodiments, a target of this disclosure comprises or consists of a sufficient number of amino acids to be specifically bound by a binding domain, such as having at least 5, 6, 7, 8, about 9, about 10, about 11, about 12, about 13, about 14, about 15, about 20, about 30, about 40, about 50, about 60, about 70, about 80, about 90, or about 100 amino acids of a sequence of any target provided herein.

In some embodiments, a binding protein of this disclosure comprises an anti-target antibody, an anti-target fusion protein, or an antigen-binding fragment thereof. Exemplary anti-target binding proteins include one or more of belimumab, tabalumab, rozibafusp alfa (also known as AMG-570; two tandem copies of BAFF-binding peptides fused to the C-terminus of anti-ICOSL mAb heavy chain), blisibimod (also known as AMG-623; BAFF binding domain fused to the N-terminus of hIgG1), atacicept (TACI ectodomain fused to hIgG1 Fc), briobacept (extracellular ligand binding portion of BAFF-R fused to hIgG1 Fc), tibulizumab (anti-IL-17 scFv derived from ixekizumab fused by Gly-rich linker to anti-BAFF tabalumab), ALPN-303 (high affinity variant form of TACI fused to hIgG), ianalumab (also known as VAY736), vedolizumab, etrolizumab, bleselumab (also known as 4D11, ASKP-1240, ASKP 1240), dacetuzumab, giloralimab, iscalimab (also known as (CFZ-533, NVP-CFZ533, OM11-62MF), lucatumumab, mitazalimab, ravagalimab (also known as ABBV-323), selicrelumab, sotigalimab, vanalimab, BI 655064, ruplizumab, toralizumab, dapirolizumab pegol (also known as CDP7657), letolizumab (also known as BMS-986004, domain antibody targeting CD40L fused at C-terminus to IgG1 Fc), dazodalibep, abatacept (CTLA4 fused with hIgG1 Fc), belatacept (high affinity CTLA4 fused with hIgG1 Fc), CTLA4-Ig (ASP2408), CTLA4-Ig (ASP2409), alomfilimab, feladilimab, izuralimab (bispecific mixed mAb and scFv that targets ICOS and PD-1), vopratelimab, acazicolcept (also known as ALPN-101, ICOSL variant fused to null huFc), lulizumab (also known as BMS-931699 or 1h-239-891(D70C)), abrilumab, natalizumab, ontamalimab, adalimumab, etanercept, golimumab, infliximab, certolizumab pegol, sibeprenlimab (also known as VIS-649), and BION-1301.

In some embodiments, a binding protein conjugate of this disclosure comprises a compound of the present disclosure linked to a BAFF binding protein, wherein the binding protein comprises a binding domain, an antibody, an antibody construct, a fusion protein, or a targeting moiety that specifically binds to BAFF. Such anti-BAFF binding proteins, and GR agonist conjugates thereof, of this disclosure are capable of specifically binding to BAFF expressing cells. In certain embodiments, an anti-BAFF binding protein, or GR agonist conjugate thereof, of this disclosure specifically binds human BAFF (see, e.g., www.uniprot.org/uniprot/Q9Y275, the sequence of which is incorporated herein by reference in its entirety) or to an epitope thereof. In certain other embodiments, a fusion protein conjugate of this disclosure comprises human TACI or an extracellular portion thereof (see, e.g., www.uniprot.org/uniprot/O14836, the sequence of which is incorporated herein by reference in its entirety). In further embodiments, an anti-BAFF binding protein, or GR agonist conjugate thereof, of this disclosure comprises an anti-BAFF antibody (e.g., belimumab or tabalumab) or an anti-BAFF fusion protein (e.g., ALPN-303 (high-affinity variant form of TACI ectodomain fused to human IgG)).

In another embodiment, the binding domain of the targeting protein also binds non-cell bound soluble target and imparts decreased inflammation and/or autoimmunity (e.g., soluble BAFF, April, TNFα).

By way of background, BAFF receptor (BAFF-R) is expressed on B lymphocytes, and when activated by BAFF alone, results in increased B-cell survival and autoimmune signaling through NF-κB. When an anti-BAFF binding protein conjugate of this disclosure binds to BAFF, the conjugate interrupts this signaling pathway and effectively blocks BAFF-R by binding to both membrane-bound and soluble BAFF. Thus, one result of administration of an anti-BAFF conjugate of this disclosure is to inhibit B-cell activation through BAFF-R. Simultaneously, myeloid cells internalize the anti-BAFF conjugate through micropinocytosis, Fc receptor mediated uptake or both. Once internalized by the myeloid cell, the anti-BAFF conjugate releases its GR or GM agonist payload within the myeloid cell altering cell surface and soluble molecule production (e.g., cytokines and chemokines) that affect immune activation of nearby cells such as T cells. In addition, the anti-BAFF conjugates of this disclosure may also secondarily inhibit T cells and other immune cells adjacent to the dendritic cells (or myeloid cells) due to bystander activity (i.e., release of payload by dendritic cells that then subsequently inhibits GR signaling in T cells). In some embodiments, a conjugate of this disclosure comprises an anti-BAFF binding protein that diminishes pathogenic B cell activity, such as belimumab or tabalumab or antigen binding domains thereof, and a payload that delivers disease suppressive GR agonism to myeloid cells and to other cell types (such as plasmacytoid dendritic, or T cells).

In some embodiments, a binding protein conjugate of this disclosure comprises a GR agonist linked to an anti-BAFF antibody, wherein the anti-BAFF antibody comprises heavy chain CDR amino acid sequences of CDR1 (VH-CDR1), VH-CDR2, and VH-CDR3 from belimumab and light chain CDR amino acid sequences of CDR1 (VL-CDR1), VL-CDR2, and VL-CDR3 from belimumab. In certain embodiments, a conjugate of this disclosure comprises an anti-BAFF antibody having a heavy chain variable (VH) region comprising an amino acid sequence that is at least about 90%, 91%, 92%, 93%, 94%, 95%, 96%, 97%, 98%, 99%, or 100% identical to the belimumab VH region amino acid sequence, and a light chain variable (VL) region comprising an amino acid sequence that is at least 90%, 91%, 92%, 93%, 94%, 95%, 96%, 97%, 98%, 99%, or 100% identical to the belimumab VL region amino acid sequence. In other embodiments, a conjugate of this disclosure comprises an anti-BAFF antibody having a heavy chain comprising an amino acid sequence that is at least 90%, 91%, 92%, 93%, 94%, 95%, 96%, 97%, 98%, 99%, or 100% identical to the belimumab heavy chain amino acid sequence, and a light chain comprising an amino acid sequence that is at least 90%, 91%, 92%, 93%, 94%, 95%, 96%, 97%, 98%, 99%, or 100% identical to the belimumab light chain amino acid sequence.

In some embodiments, a conjugate of this disclosure comprises an anti-BAFF antibody, wherein the anti-BAFF antibody comprises heavy chain CDR amino acid sequences of VH-CDR1, VH-CDR2, and VH-CDR3 from tabalumab and light chain CDR amino acid sequences of VL-CDR1, VL-CDR2, and VL-CDR3 from tabalumab. In certain embodiments, a conjugate of this disclosure comprises an anti-BAFF antibody having a VH region comprising an amino acid sequence that is at least 90%, 91%, 92%, 93%, 94%, 95%, 96%, 97%, 98%, 99%, or 100% identical to the tabalumab VH amino acid sequence, and a VL region comprising an amino acid sequence that is at least 90%, 91%, 92%, 93%, 94%, 95%, 96%, 97%, 98%, 99%, or 100% identical to the tabalumab VL amino acid sequence. In other embodiments, a conjugate of this disclosure comprises an anti-BAFF antibody having a heavy chain comprising an amino acid sequence that is at least 90%, 91%, 92%, 93%, 94%, 95%, 96%, 97%, 98%, 99%, or 100% identical to the tabalumab heavy chain amino acid sequence, and a light chain comprising an amino acid sequence that is at least 90%, 91%, 92%, 93%, 94%, 95%, 96%, 97%, 98%, 99%, or 100% identical to the tabalumab light chain amino acid sequence.

In further embodiments, a conjugate of this disclosure comprises a compound of the present disclosure linked to a bispecific antibody construct comprised of an antibody specific for BAFF and a binding domain specific for IL-17, wherein the binding domain specific for IL-17 is linked via a peptide spacer to the anti-BAFF antibody heavy chain and the peptide spacer linking the binding domain specific for IL-17 and anti-ICOSL antibody comprises from about five to about 15 amino acids (preferably 14 amino acids) and the binding domain specific for IL-17 comprises an scFv including the VH and VL regions from ixekizumab linked via a second peptide spacer comprising from about ten to about 30 amino acids (preferably 20 amino acids). In any of these conjugate embodiments, a BAFF binding peptide comprises the anti-BAFF binding domain from tabalumab. In certain embodiments, a conjugate of this disclosure comprises a GR agonist linked to a fusion protein comprising a heavy chain amino acid sequence that is at least 90%, 91%, 92%, 93%, 94%, 95%, 96%, 97%, 98%, 99%, or 100% identical to the tibulizumab heavy chain amino acid sequence and a light chain amino acid sequence that is at least 90%, 91%, 92%, 93%, 94%, 95%, 96%, 97%, 98%, 99%, or 100% identical to the tibulizumab light chain amino acid sequence.

In some embodiments, a conjugate of this disclosure comprises a GR agonist linked to an anti-BAFF binding protein, wherein the binding protein comprises a BAFF-binding peptide that specifically binds to BAFF. In further embodiments, a conjugate of this disclosure comprises a GR agonist linked to a bispecific antibody construct comprised of a BAFF binding peptide and an antibody specific for inducible co-stimulator ligand (ICOSL), wherein one or more BAFF binding peptide are linked via a peptide spacer to the anti-ICOSL antibody heavy chain and the peptide spacer linking the BAFF binding peptide and anti-ICOSL antibody comprises from about five to about 15 amino acids and the two or more BAFF binding peptide are linked via a second peptide spacer comprising from about 15 to about 35 amino acids. In any of these conjugate embodiments, a BAFF binding peptide comprises the BAFF binding peptide from rozibafusp alfa. In certain embodiments, a conjugate of this disclosure comprises a GR agonist linked to a fusion protein comprising an amino acid sequence that is at least 90%, 91%, 92%, 93%, 94%, 95%, 96%, 97%, 98%, 99%, or 100% identical to rozibafusp alfa.

In some embodiments, a conjugate of this disclosure comprises an anti-BAFF fusion protein comprising one or both blisibimod BAFF-binding peptide amino acid sequences fused to an Fc region portion. In certain embodiments, a conjugate of this disclosure comprises an anti-BAFF fusion protein having an amino acid sequence that is at least 90%, 91%, 92%, 93%, 94%, 95%, 96%, 97%, 98%, 99%, or 100% identical to the blisibimod amino acid sequence.

In some embodiments, a conjugate of this disclosure comprises a compound of the present disclosure linked to an anti-BAFF binding protein, wherein the binding protein comprises a TACI ectodomain or portion thereof that specifically binds to BAFF. In further embodiments, a conjugate of this disclosure comprises a GR agonist linked to a bispecific binding fusion protein comprising a TACI ectodomain or portion thereof that specifically binds to BAFF and APRIL. In certain embodiments, a conjugate of this disclosure comprises a GR agonist linked to a fusion protein comprised of a TACI ectodomain and an Fc region portion, wherein the TACI ectodomain is optionally fused to the Fc region portion via a peptide spacer comprised of about five to about 15 amino acids. In any of these conjugate embodiments, a BAFF binding peptide or BAFF and APRIL binding peptide comprises the BAFF or BAFF and APRIL binding peptide from atacicept or ALPN-303. In some embodiments, a conjugate of this disclosure comprises a GR agonist linked to a fusion protein comprising an amino acid sequence that is at least 90%, 91%, 92%, 93%, 94%, 95%, 96%, 97%, 98%, 99%, or 100% identical to the atacicept amino acid sequence or the ALPN-303 amino acid sequence.

In some embodiments, a conjugate of this disclosure comprises a compound of the present disclosure linked to an anti-BAFF binding protein, wherein the binding protein comprises a BAFF-R ectodomain or portion thereof that specifically binds to BAFF. In further embodiments, a conjugate of this disclosure comprises a compound of the present disclosure linked to a fusion protein comprised of a BAFF-R ectodomain and an Fc region portion, such as a human IgG1 Fc region, wherein the TACI ectodomain is optionally fused to the Fc region portion via a peptide spacer comprised of about five to about 15 amino acids. In certain embodiments, an anti-BAFF fusion protein comprises an amino acid sequence that is at least 90%, 91%, 92%, 93%, 94%, 95%, 96%, 97%, 98%, 99%, or 100% identical to the briobacept amino acid sequence.

In certain embodiments, a binding protein conjugate of this disclosure comprises a compound of the present disclosure linked to a BAFF-R binding protein, wherein the binding protein comprises a binding domain, an antibody, an antibody construct, a fusion protein, or a targeting moiety that specifically binds to BAFF-R. Such anti-BAFF-R binding proteins and GR agonist conjugates thereof of the disclosure are capable of specifically binding to BAFF-R expressing cells. In certain embodiments, an anti-BAFF-R binding protein, or conjugate thereof, of this disclosure specifically binds human BAFF-R (see, e.g., www.uniprot.org/uniprot/Q96RJ3, the sequence of which is incorporated herein by reference in its entirety) or to an epitope thereof. In further embodiments, an anti-BAFF-R binding protein, or conjugate thereof, of this disclosure comprises an anti-BAFF-R antibody (e.g., ianalumab) or an anti-BAFF-R fusion protein.

In some embodiments, a conjugate of this disclosure comprises an anti-BAFF antibody, wherein the anti-BAFF-R antibody comprises heavy chain CDR amino acid sequences of VH-CDR1, VH-CDR2, and VH-CDR3 from ianalumab and light chain CDR amino acid sequences of VL-CDR1, VL-CDR2, and VL-CDR3 from ianalumab. In certain embodiments, a conjugate of this disclosure comprises an anti-BAFF-R antibody having a VH region comprising an amino acid sequence that is at least 90%, 91%, 92%, 93%, 94%, 95%, 96%, 97%, 98%, 99%, or 100% identical to the ianalumab VH amino acid sequence, and a VL region comprising an amino acid sequence that is at least 90%, 91%, 92%, 93%, 94%, 95%, 96%, 97%, 98%, 99%, or 100% identical to the ianalumab VL amino acid sequence. In other embodiments, a conjugate of this disclosure comprises an anti-BAFF-R antibody having a heavy chain comprising an amino acid sequence that is at least 90%, 91%, 92%, 93%, 94%, 95%, 96%, 97%, 98%, 99%, or 100% identical to the ianalumab heavy chain amino acid sequence, and a light chain comprising an amino acid sequence that is at least 90%, 91%, 92%, 93%, 94%, 95%, 96%, 97%, 98%, 99%, or 100% identical to the ianalumab light chain amino acid sequence.

In certain embodiments, a binding protein conjugate of this disclosure comprises a compound of the present disclosure linked to a TACI binding protein, wherein the binding protein comprises a binding domain, an antibody, an antibody construct, a fusion protein, or a targeting moiety that specifically binds to TACI. Such anti-TACI binding domains and conjugates of the disclosure are capable of specifically binding to TACI expressing cells. In certain embodiments, an anti-TACI binding protein, or GR agonist conjugate thereof, of this disclosure specifically binds human TACI (see, e.g., Uniprot #014836, the sequence of which is incorporated herein by reference in its entirety) or to an epitope thereof. In further embodiments, an anti-TACI binding protein, or GR agonist conjugate thereof, of this disclosure comprises an anti-TACI antibody or an anti-TACI fusion protein.

In certain embodiments, a binding protein conjugate of this disclosure comprises a GR agonist linked to an α4 integrin or α4β7 integrin heterodimer binding protein, wherein the binding protein comprises a binding domain, an antibody, an antibody construct, a fusion protein, or a targeting moiety that specifically binds to α4 integrin or α4β7 integrin. Such anti-α4 integrin or anti-α4β7 integrin binding domains and conjugates thereof of this disclosure are capable of specifically binding to α4 integrin or α4β7 integrin expressing cells. In certain embodiments, an anti-α4β7 integrin binding protein or GR agonist conjugate thereof of this disclosure specifically binds human LPAM-1 (see, e.g., Uniprot #P26010, the sequence of which is incorporated herein by reference in its entirety) or to an epitope thereof. In certain embodiments, an anti-α4 integrin binding protein or GR agonist conjugate thereof of this disclosure specifically binds human α4 integrin (see, e.g., Uniprot #P13612, the sequence of which is incorporated herein by reference in its entirety) or to an epitope thereof. In further embodiments, an anti-LPAM-1 binding protein, or GR agonist conjugate thereof, of this disclosure comprises an anti-LPAM-1 antibody (e.g., vedolizumab or natrilizumab) or an anti-LPAM-1 fusion protein.

In certain embodiments, a binding protein conjugate of this disclosure comprises a GR agonist linked to an anti-α4 subunit of α4β7 integrin heterodimer antibody, wherein the anti-integrin antibody comprises heavy chain amino acid sequences of CDR1 (VH-CDR1), VH-CDR2, and VH-CDR3 from abrilumab and amino acid sequences of light chain CDR1 (VL-CDR1), VL-CDR2, and VL-CDR3 from abrilumab. In certain embodiments, a conjugate of this disclosure comprises an anti-α4β7 integrin antibody having a VH region comprising an amino acid sequence that is at least 90%, 91%, 92%, 93%, 94%, 95%, 96%, 97%, 98%, 99%, or 100% identical to the abrilumab VH region amino acid sequence, and a VL region comprising an amino acid sequence that is at least 90%, 91%, 92%, 93%, 94%, 95%, 96%, 97%, 98%, 99%, or 100% identical to the abrilumab VL region amino acid sequence. In other embodiments, a conjugate of this disclosure comprises an anti-α4β7 integrin antibody having a heavy chain comprising an amino acid sequence that is at least 90%, 91%, 92%, 93%, 94%, 95%, 96%, 97%, 98%, 99%, or 100% identical to the abrilumab heavy chain amino acid sequence, and a light chain comprising an amino acid sequence that is at least 90%, 91%, 92%, 93%, 94%, 95%, 96%, 97%, 98%, 99%, or 100% identical to the abrilumab light chain amino acid sequence.

In certain embodiments, a binding protein conjugate of this disclosure comprises a GR agonist linked to an anti-α4 integrin antibody, wherein the anti-α4β7 integrin antibody comprises heavy chain amino acid sequences of CDR1 (VH-CDR1), VH-CDR2, and VH-CDR3 from natalizumab and amino acid sequences of light chain CDR1 (VL-CDR1), VL-CDR2, and VL-CDR3 from natalizumab. In certain embodiments, a conjugate of this disclosure comprises an anti-α4 integrin antibody having a VH region comprising an amino acid sequence that is at least 90%, 91%, 92%, 93%, 94%, 95%, 96%, 97%, 98%, 99%, or 100% identical to the natalizumab VH region amino acid sequence, and a VL region comprising an amino acid sequence that is at least 90%, 91%, 92%, 93%, 94%, 95%, 96%, 97%, 98%, 99%, or 100% identical to the natalizumab VL region amino acid sequence. In other embodiments, a conjugate of this disclosure comprises an anti-α4 integrin antibody having a heavy chain comprising an amino acid sequence that is at least 90%, 91%, 92%, 93%, 94%, 95%, 96%, 97%, 98%, 99%, or 100% identical to the natalizumab heavy chain amino acid sequence, and a light chain comprising an amino acid sequence that is at least 90%, 91%, 92%, 93%, 94%, 95%, 96%, 97%, 98%, 99%, or 100% identical to the natalizumab light chain amino acid sequence.

In some embodiments, anti-Integrin α4 binding domains of the disclosure specifically bind to an epitope of Integrin α4 comprising at least 1, 2, 3, 4, 5, 6, 7, 8, 9, 10, 11, or 12 amino acids of at least one of the isoforms shown in Table A (www.uniprot.org/uniprot/P13612).

In particular embodiments, anti-Integrin α4 antibodies, or antigen-binding fragments thereof, of the disclosure specifically bind to an epitope of Integrin α4 comprising at least 1, 2, 3, 4, 5, 6, 7, 8, 9, 10, 11, or 12 amino acids of at least one of the isoforms shown in Table A (www.uniprot.org/uniprot/P13612). In particular embodiments, anti-Integrin α4 fusion proteins of the disclosure specifically bind to an epitope of Integrin α4 comprising at least 1, 2, 3, 4, 5, 6, 7, 8, 9, 10, 11, or 12 amino acids of at least one of the isoforms shown in Table A (www.uniprot.org/uniprot/P13612).

In some embodiments, an epitope of the disclosure is continuous. In some embodiments, an epitope of the disclosure is discontinuous. In some embodiments, an epitope of the disclosure conformational.

Additional information about Integrin α4 may be found at www.uniprot.org/uniprot/P13612, the contents of which are incorporated by reference in their entirety.

In certain embodiments, a binding protein conjugate of this disclosure comprises a GR agonist linked to a LPAM-1 binding protein, wherein the binding protein comprises a binding domain, an antibody, an antibody construct, a fusion protein, or a targeting moiety that specifically binds to LPAM-1. Such anti-LPAM-1 binding domains and conjugates of the disclosure are capable of specifically binding to LPAM-1 expressing cells. In certain embodiments, an anti-LPAM-1 binding protein, or GR agonist conjugate thereof, of this disclosure specifically binds human LPAM-1 (see, e.g., www.uniprot.org/uniprot/P26010, the sequence of which is incorporated herein by reference in its entirety) or to an epitope thereof. In further embodiments, an anti-LPAM-1 binding protein, or GR agonist conjugate thereof, of this disclosure comprises an anti-LPAM-1 antibody (e.g., vedolizumab or etrolizumab) or an anti-LPAM-1 fusion protein.

By way of background, LPAM-1 is most highly expressed on lymphocyte subsets and mediates lymphocyte attachment to mucosal high endothelial venules and homing to the gastrointestinal tract through interaction with MADCAM-1. Lymphocytes expressing LPAM-1 internalize an anti-LPAM-1 GR agonist conjugate of the disclosure through endosomal uptake and, once internalized, the anti-LPAM-1 GR agonist conjugate can release the GR agonist payload within the lymphocyte. In some embodiments, a conjugate of this disclosure comprises a binding protein that directly alters pathogenic T cell activation and the conjugate payload delivers GR agonism to the pathogenic T cells. In some embodiments, a binding protein that directly alters pathogenic T cell activation comprises a binding domain specific for LPAM-1.

In certain embodiments, a binding protein conjugate of this disclosure comprises a GR agonist linked to an anti-LPAM-1 antibody, wherein the anti-LPAM-1 antibody comprises heavy chain amino acid sequences of CDR1 (VH-CDR1), VH-CDR2, and VH-CDR3 from vedolizumab and amino acid sequences of light chain CDR1 (VL-CDR1), VL-CDR2, and VL-CDR3 from vedolizumab. In certain embodiments, a conjugate of this disclosure comprises an anti-LPAM1 antibody having a VH region comprising an amino acid sequence that is at least 90%, 91%, 92%, 93%, 94%, 95%, 96%, 97%, 98%, 99%, or 100% identical to the vedolizumab VH region amino acid sequence, and a VL region comprising an amino acid sequence that is at least 90%, 91%, 92%, 93%, 94%, 95%, 96%, 97%, 98%, 99%, or 100% identical to the vedolizumab VL region amino acid sequence. In other embodiments, a conjugate of this disclosure comprises an anti-LPAM-1 antibody having a heavy chain comprising an amino acid sequence that is at least 90%, 91%, 92%, 93%, 94%, 95%, 96%, 97%, 98%, 99%, or 100% identical to the vedolizumab heavy chain amino acid sequence, and a light chain comprising an amino acid sequence that is at least 90%, 91%, 92%, 93%, 94%, 95%, 96%, 97%, 98%, 99%, or 100% identical to the vedolizumab light chain amino acid sequence.

In some embodiments, a conjugate of this disclosure comprises an anti-LPAM-1 antibody or anti-β7 subunit of α4β7 or αEβ7 integrin heterodimers, wherein the anti-LPAM-1 or anti-β7 subunit antibody comprises heavy chain CDR amino acid sequences of VH-CDR1, VH-CDR2, and VH-CDR3 from etrolizumab and light chain CDR amino acid sequences of VL-CDR1, VL-CDR2, and VL-CDR3 from etrolizumab. In certain embodiments, a conjugate of this disclosure comprises an anti-LPAM-1 or anti-β7 subunit antibody having a VH comprising an amino acid sequence that is at least 90%, 91%, 92%, 93%, 94%, 95%, 96%, 97%, 98%, 99%, or 100% identical to the etrolizumab VH amino acid sequence, and a VL region comprising an amino acid sequence that is at least 90%, 91%, 92%, 93%, 94%, 95%, 96%, 97%, 98%, 99%, or 100% identical to the etrolizumab VL amino acid sequence. In other embodiments, a conjugate of this disclosure comprises an anti-LPAM-1 or anti-β7 subunit antibody having a heavy chain comprising an amino acid sequence that is at least 90%, 91%, 92%, 93%, 94%, 95%, 96%, 97%, 98%, 99%, or 100% identical to the etrolizumab heavy chain amino acid sequence, and a light chain comprising an amino acid sequence that is at least 90%, 91%, 92%, 93%, 94%, 95%, 96%, 97%, 98%, 99%, or 100% identical to the etrolizumab light chain amino acid sequence.

In certain embodiments, a binding protein conjugate of this disclosure comprises a GR agonist linked to a BCMA binding protein, wherein the binding protein comprises a binding domain, an antibody, an antibody construct, a fusion protein, or a targeting moiety that specifically binds to BCMA. Such anti-BCMA binding domains and conjugates of the disclosure are capable of specifically binding to BCMA expressing cells. In certain embodiments, an anti-BCMA binding protein, or GR agonist conjugate thereof, of this disclosure specifically binds human BCMA (see, e.g., www.uniprot.org/uniprot/Q02223, the sequence of which is incorporated herein by reference in its entirety) or to an epitope thereof. In further embodiments, an anti-BCMA binding protein, or GR agonist conjugate thereof, of this disclosure comprises an anti-BCMA antibody or an anti-BCMA fusion protein.

In some embodiments, a conjugate of this disclosure comprises an anti-BCMA antibody, wherein the anti-BCMA antibody comprises heavy chain CDR amino acid sequences of VH-CDR1, VH-CDR2, and VH-CDR3 from belantamab and light chain CDR amino acid sequences of VL-CDR1, VL-CDR2, and VL-CDR3 from belantamab. In certain embodiments, a conjugate of this disclosure comprises an anti-BCMA antibody having a VH region comprising an amino acid sequence that is at least 90%, 91%, 92%, 93%, 94%, 95%, 96%, 97%, 98%, 99%, or 100% identical to the belantamab VH amino acid sequence, and a VL region comprising an amino acid sequence that is at least 90%, 91%, 92%, 93%, 94%, 95%, 96%, 97%, 98%, 99%, or 100% identical to the belantamab VL amino acid sequence. In other embodiments, a conjugate of this disclosure comprises an anti-BCMA antibody having a heavy chain comprising an amino acid sequence that is at least 90%, 91%, 92%, 93%, 94%, 95%, 96%, 97%, 98%, 99%, or 100% identical to the belantamab heavy chain amino acid sequence, and a light chain comprising an amino acid sequence that is at least 90%, 91%, 92%, 93%, 94%, 95%, 96%, 97%, 98%, 99%, or 100% identical to the belantamab light chain amino acid sequence.

In certain embodiments, a binding protein conjugate of this disclosure comprises a compound of the present disclosure linked to a CD40 binding protein, wherein the binding protein comprises a binding domain, an antibody, an antibody construct, a fusion protein, or a targeting moiety that specifically binds to CD40. Such anti-CD40 binding domains and conjugates of the disclosure are capable of specifically binding to CD40 expressing cells (e.g., antigen presenting cells, including B-cells). In certain embodiments, an anti-CD40 binding protein, or conjugate thereof, of this disclosure specifically binds human CD40 (see, e.g., www.uniprot.org/uniprot/P25942, the sequence of which is incorporated herein by reference in its entirety) or to an epitope thereof. In further embodiments, an anti-CD40 binding protein, or conjugate thereof, of this disclosure comprises an anti-CD40 antibody or an anti-CD40 fusion protein.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from bleselumab and light chain CDR amino acid sequences of VL-CDR1, VL-CDR2, and VL-CDR3 from bleselumab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the bleselumab VH amino acid sequence, and a VL region comprising an amino acid sequence that is at least 90%, 91%, 92%, 93%, 94%, 95%, 96%, 97%, 98%, 99%, or 100% identical to the bleselumab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the bleselumab heavy chain amino acid sequence, and a light chain comprising an amino acid sequence that is at least 90%, 91%, 92%, 93%, 94%, 95%, 96%, 97%, 98%, 99%, or 100% identical to the bleselumab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from dacetuzumab and light chain CDR amino acid sequences of VL-CDR1, VL-CDR2, and VL-CDR3 from dacetuzumab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the dacetuzumab VH amino acid sequence, and a VL region comprising an amino acid sequence that is at least 90%, 91%, 92%, 93%, 94%, 95%, 96%, 97%, 98%, 99%, or 100% identical to the dacetuzumab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the dacetuzumab heavy chain amino acid sequence, and a light chain comprising an amino acid sequence that is at least 90%, 91%, 92%, 93%, 94%, 95%, 96%, 97%, 98%, 99%, or 100% identical to the dacetuzumab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from giloralimab and light chain CDR amino acid sequences of VL-CDR1, VL-CDR2, and VL-CDR3 from giloralimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the giloralimab VH amino acid sequence, and a VL region comprising an amino acid sequence that is at least 90%, 91%, 92%, 93%, 94%, 95%, 96%, 97%, 98%, 99%, or 100% identical to the giloralimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the giloralimab heavy chain amino acid sequence, and a light chain comprising an amino acid sequence that is at least 90%, 91%, 92%, 93%, 94%, 95%, 96%, 97%, 98%, 99%, or 100% identical to the giloralimab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from iscalimab and light chain CDR amino acid sequences of VL-CDR1, VL-CDR2, and VL-CDR3 from iscalimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the iscalimab VH amino acid sequence, and a VL region comprising an amino acid sequence that is at least 90%, 91%, 92%, 93%, 94%, 95%, 96%, 97%, 98%, 99%, or 100% identical to the iscalimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the iscalimab heavy chain amino acid sequence, and a light chain comprising an amino acid sequence that is at least 90%, 91%, 92%, 93%, 94%, 95%, 96%, 97%, 98%, 99%, or 100% identical to the iscalimab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from lucatumumab and light chain CDR amino acid sequences of VL-CDR1, VL-CDR2, and VL-CDR3 from lucatumumab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the lucatumumab VH amino acid sequence, and a VL region comprising an amino acid sequence that is at least 90%, 91%, 92%, 93%, 94%, 95%, 96%, 97%, 98%, 99%, or 100% identical to the lucatumumab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the lucatumumab heavy chain amino acid sequence, and a light chain comprising an amino acid sequence that is at least 90%, 91%, 92%, 93%, 94%, 95%, 96%, 97%, 98%, 99%, or 100% identical to the lucatumumab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from mitazalimab and light chain CDR amino acid sequences of VL-CDR1, VL-CDR2, and VL-CDR3 from mitazalimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the mitazalimab VH amino acid sequence, and a VL region comprising an amino acid sequence that is at least 90%, 91%, 92%, 93%, 94%, 95%, 96%, 97%, 98%, 99%, or 100% identical to the mitazalimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the mitazalimab heavy chain amino acid sequence, and a light chain comprising an amino acid sequence that is at least 90%, 91%, 92%, 93%, 94%, 95%, 96%, 97%, 98%, 99%, or 100% identical to the mitazalimab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from ravagalimab and light chain CDR amino acid sequences of VL-CDR1, VL-CDR2, and VL-CDR3 from ravagalimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the ravagalimab VH amino acid sequence, and a VL region comprising an amino acid sequence that is at least 90%, 91%, 92%, 93%, 94%, 95%, 96%, 97%, 98%, 99%, or 100% identical to the ravagalimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the ravagalimab heavy chain amino acid sequence, and a light chain comprising an amino acid sequence that is at least 90%, 91%, 92%, 93%, 94%, 95%, 96%, 97%, 98%, 99%, or 100% identical to the ravagalimab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from selicrelumab and light chain CDR amino acid sequences of VL-CDR1, VL-CDR2, and VL-CDR3 from selicrelumab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the selicrelumab VH amino acid sequence, and a VL region comprising an amino acid sequence that is at least 90%, 91%, 92%, 93%, 94%, 95%, 96%, 97%, 98%, 99%, or 100% identical to the selicrelumab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the selicrelumab heavy chain amino acid sequence, and a light chain comprising an amino acid sequence that is at least 90%, 91%, 92%, 93%, 94%, 95%, 96%, 97%, 98%, 99%, or 100% identical to the selicrelumab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from sotigalimab and light chain CDR amino acid sequences of VL-CDR1, VL-CDR2, and VL-CDR3 from sotigalimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the sotigalimab VH amino acid sequence, and a VL region comprising an amino acid sequence that is at least 90%, 91%, 92%, 93%, 94%, 95%, 96%, 97%, 98%, 99%, or 100% identical to the sotigalimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the sotigalimab heavy chain amino acid sequence, and a light chain comprising an amino acid sequence that is at least 90%, 91%, 92%, 93%, 94%, 95%, 96%, 97%, 98%, 99%, or 100% identical to the sotigalimab light chain amino acid sequence.

In some embodiments, a conjugate of this disclosure comprises an anti-CD40 antibody, wherein the anti-CD40 antibody comprises heavy chain CDR amino acid sequences of VH-CDR1, VH-CDR2, and VH-CDR3 from vanalimab and light chain CDR amino acid sequences of VL-CDR1, VL-CDR2, and VL-CDR3 from vanalimab. In certain embodiments, a conjugate of this disclosure comprises an anti-CD40 antibody having a VH region comprising an amino acid sequence that is at least 90%, 91%, 92%, 93%, 94%, 95%, 96%, 97%, 98%, 99%, or 100% identical to the vanalimab VH amino acid sequence, and a VL region comprising an amino acid sequence that is at least 90%, 91%, 92%, 93%, 94%, 95%, 96%, 97%, 98%, 99%, or 100% identical to the vanalimab VL amino acid sequence. In other embodiments, a conjugate of this disclosure comprises an anti-CD40 antibody having a heavy chain comprising an amino acid sequence that is at least 90%, 91%, 92%, 93%, 94%, 95%, 96%, 97%, 98%, 99%, or 100% identical to the vanalimab heavy chain amino acid sequence, and a light chain comprising an amino acid sequence that is at least 90%, 91%, 92%, 93%, 94%, 95%, 96%, 97%, 98%, 99%, or 100% identical to the vanalimab light chain amino acid sequence.

In certain embodiments, a binding protein conjugate of this disclosure comprises a GR agonist linked to a CD40L binding protein, wherein the binding protein comprises a binding domain, an antibody, an antibody construct, a fusion protein, or a targeting moiety that specifically binds to CD40L. Such anti-CD40L binding domains and conjugates of the disclosure are capable of specifically binding to CD40L expressing cells. In certain embodiments, an anti-CD40L binding protein, or GR agonist conjugate thereof, of this disclosure specifically binds human CD40L (see, e.g., www.uniprot.org/uniprot/P25942, the sequence of which is incorporated herein by reference in its entirety) or to an epitope thereof. In further embodiments, an anti-CD40L binding protein, or GR agonist conjugate thereof, of this disclosure comprises an anti-CD40L antibody or an anti-CD40L fusion protein.

In some embodiments, a conjugate of this disclosure comprises an anti-CD40L antibody, wherein the anti-CD40L antibody comprises heavy chain CDR amino acid sequences of VH-CDR1, VH-CDR2, and VH-CDR3 from ruplizumab and light chain CDR amino acid sequences of VL-CDR1, VL-CDR2, and VL-CDR3 from ruplizumab. In certain embodiments, a conjugate of this disclosure comprises an anti-CD40L antibody having a VH region comprising an amino acid sequence that is at least 90%, 91%, 92%, 93%, 94%, 95%, 96%, 97%, 98%, 99%, or 100% identical to the ruplizumab VH amino acid sequence, and a VL region comprising an amino acid sequence that is at least 90%, 91%, 92%, 93%, 94%, 95%, 96%, 97%, 98%, 99%, or 100% identical to the ruplizumab VL amino acid sequence. In other embodiments, a conjugate of this disclosure comprises an anti-CD40L antibody having a heavy chain comprising an amino acid sequence that is at least 90%, 91%, 92%, 93%, 94%, 95%, 96%, 97%, 98%, 99%, or 100% identical to the ruplizumab heavy chain amino acid sequence, and a light chain comprising an amino acid sequence that is at least 90%, 91%, 92%, 93%, 94%, 95%, 96%, 97%, 98%, 99%, or 100% identical to the ruplizumab light chain amino acid sequence.

In some embodiments, a conjugate of this disclosure comprises an anti-CD40L antibody, wherein the anti-CD40L antibody comprises heavy chain CDR amino acid sequences of VH-CDR1, VH-CDR2, and VH-CDR3 from toralizumab and light chain CDR amino acid sequences of VL-CDR1, VL-CDR2, and VL-CDR3 from toralizumab. In certain embodiments, a conjugate of this disclosure comprises an anti-CD40L antibody having a VH region comprising an amino acid sequence that is at least 90%, 91%, 92%, 93%, 94%, 95%, 96%, 97%, 98%, 99%, or 100% identical to the toralizumab VH amino acid sequence, and a VL region comprising an amino acid sequence that is at least 90%, 91%, 92%, 93%, 94%, 95%, 96%, 97%, 98%, 99%, or 100% identical to the toralizumab VL amino acid sequence. In other embodiments, a conjugate of this disclosure comprises an anti-CD40L antibody having a heavy chain comprising an amino acid sequence that is at least 90%, 91%, 92%, 93%, 94%, 95%, 96%, 97%, 98%, 99%, or 100% identical to the toralizumab heavy chain amino acid sequence, and a light chain comprising an amino acid sequence that is at least 90%, 91%, 92%, 93%, 94%, 95%, 96%, 97%, 98%, 99%, or 100% identical to the toralizumab light chain amino acid sequence.

In some embodiments, a conjugate of this disclosure comprises an anti-CD40L binding domain, wherein the anti-CD40L binding domain comprises heavy chain CDR amino acid sequences of VH-CDR1, VH-CDR2, and VH-CDR3 from dapirolizumab and light chain CDR amino acid sequences of VL-CDR1, VL-CDR2, and VL-CDR3 from dapirolizumab. In certain embodiments, a conjugate of this disclosure comprises an anti-CD40L binding domain having a VH region comprising an amino acid sequence that is at least 90%, 91%, 92%, 93%, 94%, 95%, 96%, 97%, 98%, 99%, or 100% identical to the dapirolizumab VH amino acid sequence, and a VL region comprising an amino acid sequence that is at least 90%, 91%, 92%, 93%, 94%, 95%, 96%, 97%, 98%, 99%, or 100% identical to the dapirolizumab VL amino acid sequence, optionally the a conjugate of this disclosure comprises a pegylated anti-CD40L binding domain. In further embodiments, a conjugate of this disclosure comprises a pegylated anti-CD40L binding domain having an amino acid sequence that is at least 90%, 91%, 92%, 93%, 94%, 95%, 96%, 97%, 98%, 99%, or 100% identical to dapirolizumab pegol.

In some embodiments, a conjugate of this disclosure comprises an anti-CD40L fusion protein, wherein the fusion protein comprises an anti-CD40L binding domain having heavy chain CDR amino acid sequences of VH-CDR1, VH-CDR2, and VH-CDR3 from letolizumab fused to an Fc region portion. In certain embodiments, a conjugate of this disclosure comprises an anti-CD40L fusion protein having a VH region comprising an amino acid sequence that is at least 90%, 91%, 92%, 93%, 94%, 95%, 96%, 97%, 98%, 99%, or 100% identical to the letolizumab VH amino acid sequence fused to an Fc region portion.

In some embodiments, a conjugate of this disclosure comprises an anti-CD40L fusion protein comprising one or both dazodalibep anti-CD40L binding peptide amino acid sequences fused to human serum albumin (HSA), wherein the dazodalibep anti-CD40L binding peptides are linked via a peptide spacer from about five to about 20 amino acids to the HSA (preferably 10 amino acids) and the anti-CD40L binding peptides are linked via a second peptide spacer comprising from about five to about 30 amino acids (preferably 20 amino acids). In certain embodiments, a conjugate of this disclosure comprises an anti-CD40L fusion protein having an amino acid sequence that is at least 90%, 91%, 92%, 93%, 94%, 95%, 96%, 97%, 98%, 99%, or 100% identical to the dazodalibep amino acid sequence.

In certain embodiments, a binding protein conjugate of this disclosure comprises a GR agonist linked to a CD86 binding protein or a CD80 binding protein, wherein the binding protein comprises a binding domain, an antibody, an antibody construct, a fusion protein, or a targeting moiety that specifically binds to CD86, CD80, or both. Such CD86 or CD80 binding domains and conjugates thereof of this disclosure are capable of specifically binding to cells expressing CD86, CD80, or both. In certain embodiments, a binding protein or GR agonist conjugate thereof of this disclosure specifically binds human CD86 (see, e.g., www.uniprot.org/uniprot/P42081, the sequence of which is incorporated herein by reference in its entirety), human CD80 (see, e.g., www.uniprot.org/uniprot/P33681, the sequence of which is incorporated herein by reference in its entirety), both human CD86 and human CD80, or to an epitope thereof. In further embodiments, a fusion protein conjugate of this disclosure comprises human CTLA4 or an extracellular portion thereof (see, e.g., www.uniprot.org/uniprot/P16410, the sequence of which is incorporated herein by reference in its entirety). In further embodiments, a CD86 or CD80 binding protein, or GR agonist conjugate thereof, of this disclosure comprises an anti-CD86 antibody, an anti-CD80 antibody, or a CTLA4 fusion protein (e.g., abatacept, belatacept, ASP2408, or ASP2409).

By way of background, when a CTLA4 fusion protein conjugate (e.g., CTLA4-Ig conjugate) of this disclosure binds to CD80 or CD86, which are receptors for both CD28 and CTLA4, the conjugate interrupts the CD28 signaling pathway and effectively blocks T-cell activation. Thus, one result of administration of a CTLA4 fusion protein conjugate of this disclosure is to inhibit T-cell activation through the binding of CD80 or CD86. Simultaneously, APCs expressing CD80 or CD86 internalize the CTLA4 fusion protein conjugate through endosomal uptake and, once internalized, the GR agonist payload is released within the APC. In some embodiments, these two actions not only occur simultaneously within the T-cell and APC, but also provide a synergistic combination of results for treating inflammatory or autoimmune conditions. In some embodiments, a conjugate of this disclosure comprises a binding protein that directly alters T cell activation and the conjugate GR agonist payload delivers immune suppressive GR agonism to antigen presenting cells, including myeloid antigen presenting cells, through ligand binding or macropinocytosis. In certain embodiments, a binding protein that directly alters T cell activation comprises a binding domain specific for CD80, CD86, or both.

In some embodiments, a conjugate of this disclosure comprises an anti-CD86 or anti-CD80 fusion protein comprising a CTLA ectodomain fused to an Fc region portion. In certain embodiments, a conjugate of this disclosure comprises a CTLA ectodomain having an amino acid sequence that is at least 90%, 91%, 92%, 93%, 94%, 95%, 96%, 97%, 98%, 99%, or 100% identical to the CTLA ectodomain amino acid sequence from belatacept, abatacept, ASP2408, or ASP2409. In further embodiments, a conjugate of this disclosure comprises an anti-CD86 or anti-CD80 fusion protein having an amino acid sequence that is at least 90%, 91%, 92%, 93%, 94%, 95%, 96%, 97%, 98%, 99%, or 100% identical to the belatacept, abatacept, ASP2408, or ASP2409 amino acid sequence.

In certain embodiments, a binding protein conjugate of this disclosure comprises a GR agonist linked to an anti-CTLA4 binding protein, wherein the binding protein comprises a binding domain, an antibody, an antibody construct, a fusion protein, or a targeting moiety that specifically binds to CTLA4. Such anti-CTLA4 binding domains and conjugates of the disclosure are capable of specifically binding to CTLA4 expressing cells. In certain embodiments, an anti-CTLA4 binding protein, or GR agonist conjugate thereof, of this disclosure specifically binds human CTLA4 (see, e.g., www.uniprot.org/uniprot/P16410, the sequence of which is incorporated herein by reference in its entirety) or to an epitope thereof. In further embodiments, an anti-CTLA4 binding protein, or GR agonist conjugate thereof, of this disclosure comprises an agonistic anti-CTLA4 antibody or an agonistic anti-CTLA4 fusion protein.

By way of background, CTLA4 is expressed on activated T lymphocytes and Treg T cells. Binding by agonistic anti-CTLA4 antibody conjugates results in decreased T cell activation and increased Treg immune suppression by activating the CTLA4 signaling pathway and also via internalization and delivery of GR agonism into the T cells. In some embodiments, a conjugate of this disclosure comprises a binding protein that directly alters immune suppression to activated and Treg cells and the conjugate GR agonist payload delivers immune suppressive GR agonism to macropinocytotic myeloid cells. In certain embodiments, a binding protein that directly alters immune suppression to activated and Treg T cells comprises a binding domain specific for CTLA4.

In certain embodiments, a binding protein conjugate of this disclosure comprises a GR agonist linked to an ICOS binding protein, wherein the binding protein comprises a binding domain, an antibody, an antibody construct, a fusion protein, or a targeting moiety that specifically binds to ICOS. Such anti-ICOS binding domains and conjugates of the disclosure are capable of specifically binding to ICOS expressing cells. In certain embodiments, an anti-ICOS binding protein, or GR agonist conjugate thereof, of this disclosure specifically binds human ICOS (see, e.g., www.uniprot.org/uniprot/Q9Y6W8, the sequence of which is incorporated herein by reference in its entirety) or to an epitope thereof. In further embodiments, an anti-ICOS binding protein, or GR agonist conjugate thereof, of this disclosure comprises an agonistic anti-ICOS antibody or an agonistic anti-ICOS fusion protein.

By way of background, ICOS is expressed on T lymphocytes (T cells). ICOS ligand is expressed on B cells, macrophages and dendritic cells. When bound by ICOSL, ICOS mediates T cell proliferation and cytokine secretion. An anti-ICOS GR agonist conjugate of this disclosure will be capable of interrupting this signaling pathway and effectively block both T cell proliferation and cytokine secretion, and simultaneously deliver immune suppressive GR agonism within the ICOS expressing cell (T cell). In some embodiments, a conjugate of this disclosure comprises a binding protein that directly alters pathogenic T cell activation and the conjugate payload delivers GR agonism to the pathogenic T cells. In some embodiments, a binding protein that directly alters pathogenic T cell activation comprises a binding domain specific for ICOS.

In some embodiments, a conjugate of this disclosure comprises an anti-ICOS antibody, wherein the anti-ICOS antibody comprises heavy chain CDR amino acid sequences of VH-CDR1, VH-CDR2, and VH-CDR3 from alomfilimab and light chain CDR amino acid sequences of VL-CDR1, VL-CDR2, and VL-CDR3 from alomfilimab. In certain embodiments, a conjugate of this disclosure comprises an anti-ICOS antibody having a VH region comprising an amino acid sequence that is at least 90%, 91%, 92%, 93%, 94%, 95%, 96%, 97%, 98%, 99%, or 100% identical to the alomfilimab VH amino acid sequence, and a VL region comprising an amino acid sequence that is at least 90%, 91%, 92%, 93%, 94%, 95%, 96%, 97%, 98%, 99%, or 100% identical to the alomfilimab VL amino acid sequence. In other embodiments, a conjugate of this disclosure comprises an anti-ICOS antibody having a heavy chain comprising an amino acid sequence that is at least 90%, 91%, 92%, 93%, 94%, 95%, 96%, 97%, 98%, 99%, or 100% identical to the alomfilimab heavy chain amino acid sequence, and a light chain comprising an amino acid sequence that is at least 90%, 91%, 92%, 93%, 94%, 95%, 96%, 97%, 98%, 99%, or 100% identical to the alomfilimab light chain amino acid sequence.

In some embodiments, a conjugate of this disclosure comprises an anti-ICOS antibody, wherein the anti-ICOS antibody comprises heavy chain CDR amino acid sequences of VH-CDR1, VH-CDR2, and VH-CDR3 from feladilimab and light chain CDR amino acid sequences of VL-CDR1, VL-CDR2, and VL-CDR3 from feladilimab. In certain embodiments, a conjugate of this disclosure comprises an anti-ICOS antibody having a VH region comprising an amino acid sequence that is at least 90%, 91%, 92%, 93%, 94%, 95%, 96%, 97%, 98%, 99%, or 100% identical to the feladilimab VH amino acid sequence, and a VL region comprising an amino acid sequence that is at least 90%, 91%, 92%, 93%, 94%, 95%, 96%, 97%, 98%, 99%, or 100% identical to the feladilimab VL amino acid sequence. In other embodiments, a conjugate of this disclosure comprises an anti-ICOS antibody having a heavy chain comprising an amino acid sequence that is at least 90%, 91%, 92%, 93%, 94%, 95%, 96%, 97%, 98%, 99%, or 100% identical to the feladilimab heavy chain amino acid sequence, and a light chain comprising an amino acid sequence that is at least 90%, 91%, 92%, 93%, 94%, 95%, 96%, 97%, 98%, 99%, or 100% identical to the feladilimab light chain amino acid sequence.

In some embodiments, a conjugate of this disclosure comprises an anti-ICOS antibody, wherein the anti-ICOS antibody comprises heavy chain CDR amino acid sequences of VH-CDR1, VH-CDR2, and VH-CDR3 from vopratelimab and light chain CDR amino acid sequences of VL-CDR1, VL-CDR2, and VL-CDR3 from vopratelimab. In certain embodiments, a conjugate of this disclosure comprises an anti-ICOS antibody having a VH region comprising an amino acid sequence that is at least 90%, 91%, 92%, 93%, 94%, 95%, 96%, 97%, 98%, 99%, or 100% identical to the vopratelimab VH amino acid sequence, and a VL region comprising an amino acid sequence that is at least 90%, 91%, 92%, 93%, 94%, 95%, 96%, 97%, 98%, 99%, or 100% identical to the vopratelimab VL amino acid sequence. In other embodiments, a conjugate of this disclosure comprises an anti-ICOS antibody having a heavy chain comprising an amino acid sequence that is at least 90%, 91%, 92%, 93%, 94%, 95%, 96%, 97%, 98%, 99%, or 100% identical to the vopratelimab heavy chain amino acid sequence, and a light chain comprising an amino acid sequence that is at least 90%, 91%, 92%, 93%, 94%, 95%, 96%, 97%, 98%, 99%, or 100% identical to the vopratelimab light chain amino acid sequence.

In further embodiments, a conjugate of this disclosure comprises a GR agonist linked to a bispecific antibody construct comprised of an antibody specific for ICOS and a binding domain specific for PD-1, wherein the binding domain specific for PD-1 is optionally linked via a peptide spacer of about five to about 30 amino acids, and the binding domain specific for PD-1 comprises an scFv including the VH and VL regions linked via a peptide spacer comprising from about ten to about 30 amino acids. In certain embodiments, a conjugate of this disclosure comprises a GR agonist linked to a fusion protein comprising a heavy chain amino acid sequence that is at least 90%, 91%, 92%, 93%, 94%, 95%, 96%, 97%, 98%, 99%, or 100% identical to the izuralimab heavy chain amino acid sequence and a light chain amino acid sequence that is at least 90%, 91%, 92%, 93%, 94%, 95%, 96%, 97%, 98%, 99%, or 100% identical to the izuralimab light chain amino acid sequence.

In some embodiments, a conjugate of this disclosure comprises an anti-ICOS or anti-CD28 fusion protein comprising a ICOSL fragment fused to a null Fc region portion. In certain embodiments, a conjugate of this disclosure comprises a ICOSL fragment having an amino acid sequence that is at least 90%, 91%, 92%, 93%, 94%, 95%, 96%, 97%, 98%, 99%, or 100% identical to the ICOSL fragment amino acid sequence from acazicolcept. In further embodiments, a conjugate of this disclosure comprises an anti-ICOS or anti-CD28 fusion protein having an amino acid sequence that is at least 90%, 91%, 92%, 93%, 94%, 95%, 96%, 97%, 98%, 99%, or 100% identical to the acazicolcept amino acid sequence.

In certain embodiments, a binding protein conjugate of this disclosure comprises a GR agonist linked to an ICOSL binding protein, wherein the binding protein comprises a binding domain, an antibody, an antibody construct, a fusion protein, or a targeting moiety that specifically binds to ICOSL. Such anti-ICOSL binding domains and conjugates of the disclosure are capable of specifically binding to ICOSL expressing cells. In certain embodiments, an anti-ICOSL binding protein, or GR agonist conjugate thereof, of this disclosure specifically binds human ICOSL (see, e.g., www.uniprot.org/uniprot/O75144, the sequence of which is incorporated herein by reference in its entirety) or to an epitope thereof.

In further embodiments, an anti-ICOSL binding protein, or GR agonist conjugate thereof, of this disclosure comprises an agonistic anti-ICOSL antibody or an agonistic anti-ICOSL fusion protein.

By way of background, an anti-ICOSL conjugate or ICOS fusion protein conjugate of this disclosure will be capable of interrupting the ICOS signaling pathway and effectively block both T cell proliferation and cytokine secretion while simultaneously delivering immune suppressive GR agonism into ICOSL expressing cells (e.g., B cells, macrophages, or dendritic cells), which provides a synergistic combination of activities for treating inflammatory or autoimmune conditions.

In certain embodiments, a binding protein conjugate of this disclosure comprises a GR agonist linked to a CD28 binding protein, wherein the binding protein comprises a binding domain, an antibody, an antibody construct, a fusion protein, or a targeting moiety that specifically binds to CD28. Such anti-CD28 binding domains and conjugates of the disclosure are capable of specifically binding to CD28 expressing cells. In certain embodiments, an anti-CD28 binding protein, or GR agonist conjugate thereof, of this disclosure specifically binds human CD28 (see, e.g., www.uniprot.org/uniprot/P10747, the sequence of which is incorporated herein by reference in its entirety) or to an epitope thereof. In further embodiments, an anti-CD28 binding protein, or GR agonist conjugate thereof, of this disclosure comprises an anti-CD28 antibody or an anti-CD28 fusion protein. In some embodiments, a conjugate of this disclosure comprises a binding protein that directly alters pathogenic T cell activation and the conjugate payload delivers GR agonism to the pathogenic T cells. In some embodiments, a binding protein that directly alters pathogenic T cell activation comprises a binding domain specific for CD28.

In some embodiments, a conjugate of this disclosure comprises an anti-CD28 domain antibody, wherein the domain antibody comprises an anti-CD28 binding domain having light chain CDR amino acid sequences of VL-CDR1, VL-CDR2, and VL-CDR3 from lulizumab, optionally wherein the anti-CD28 domain antibody is pegylated. In certain embodiments, a conjugate of this disclosure comprises an anti-CD28 domain antibody having a VL region comprising an amino acid sequence that is at least 90%, 91%, 92%, 93%, 94%, 95%, 96%, 97%, 98%, 99%, or 100% identical to the lulizumab VL amino acid sequence, optionally pegylated (lulizumab pegol).

In certain embodiments, a binding protein conjugate of this disclosure comprises a GR agonist linked to an MADCAM binding protein, wherein the binding protein comprises a binding domain, an antibody, an antibody construct, a fusion protein, or a targeting moiety that specifically binds to MADCAM. Such anti-MADCAM binding domains and conjugates of the disclosure are capable of specifically binding to MADCAM expressing cells. In certain embodiments, an anti-MADCAM binding protein, or GR agonist conjugate thereof, of this disclosure specifically binds human MADCAM (see, e.g., www.uniprot.org/uniprot/Q13477, the sequence of which is incorporated herein by reference in its entirety) or to an epitope thereof. In further embodiments, an anti-MADCAM binding protein, or GR agonist conjugate thereof, of this disclosure comprises an anti-MADCAM antibody or an anti-MADCAM fusion protein.

In some embodiments, a conjugate of this disclosure comprises an anti-MADCAM antibody, wherein the anti-MADCAM antibody comprises heavy chain CDR amino acid sequences of VH-CDR1, VH-CDR2, and VH-CDR3 from ontamalimab and light chain CDR amino acid sequences of VL-CDR1, VL-CDR2, and VL-CDR3 from ontamalimab. In certain embodiments, a conjugate of this disclosure comprises an anti-MADCAM antibody having a VH region comprising an amino acid sequence that is at least 90%, 91%, 92%, 93%, 94%, 95%, 96%, 97%, 98%, 99%, or 100% identical to the ontamalimab VH amino acid sequence, and a VL region comprising an amino acid sequence that is at least 90%, 91%, 92%, 93%, 94%, 95%, 96%, 97%, 98%, 99%, or 100% identical to the ontamalimab VL amino acid sequence. In other embodiments, a conjugate of this disclosure comprises an anti-MADCAM antibody having a heavy chain comprising an amino acid sequence that is at least 90%, 91%, 92%, 93%, 94%, 95%, 96%, 97%, 98%, 99%, or 100% identical to the ontamalimab heavy chain amino acid sequence, and a light chain comprising an amino acid sequence that is at least 90%, 91%, 92%, 93%, 94%, 95%, 96%, 97%, 98%, 99%, or 100% identical to the ontamalimab light chain amino acid sequence.

In certain embodiments, a binding protein conjugate of this disclosure comprises a GR agonist linked to a TNFα binding protein, wherein the binding protein comprises a binding domain, an antibody, an antibody construct, a fusion protein, or a targeting moiety that specifically binds to TNFα. Such anti-TNFα binding domains and conjugates thereof of this disclosure are capable of specifically binding to TNFα expressing cells. In certain embodiments, an anti-TNFα binding protein, or GR agonist conjugate thereof, of this disclosure specifically binds human TNFα (see, e.g., www.uniprot.org/uniprot/P01375, the sequence of which is incorporated herein by reference in its entirety) or to an epitope thereof. In further embodiments, an anti-TNFα binding protein, or GR agonist conjugate thereof, of this disclosure comprises an anti-TNFα antibody or an anti-TNFα fusion protein.

In some embodiments, a conjugate of this disclosure comprises an anti-TNFα antibody, wherein the anti-TNFα antibody comprises heavy chain CDR amino acid sequences of VH-CDR1, VH-CDR2, and VH-CDR3 from adalimumab and light chain CDR amino acid sequences of VL-CDR1, VL-CDR2, and VL-CDR3 from adalimumab. In certain embodiments, a conjugate of this disclosure comprises an anti-TNFα antibody having a VH region comprising an amino acid sequence that is at least 90%, 91%, 92%, 93%, 94%, 95%, 96%, 97%, 98%, 99%, or 100% identical to the adalimumab VH amino acid sequence, and a VL region comprising an amino acid sequence that is at least 90%, 91%, 92%, 93%, 94%, 95%, 96%, 97%, 98%, 99%, or 100% identical to the adalimumab VL amino acid sequence. In other embodiments, a conjugate of this disclosure comprises an anti-TNFα antibody having a heavy chain comprising an amino acid sequence that is at least 90%, 91%, 92%, 93%, 94%, 95%, 96%, 97%, 98%, 99%, or 100% identical to the adalimumab heavy chain amino acid sequence, and a light chain comprising an amino acid sequence that is at least 90%, 91%, 92%, 93%, 94%, 95%, 96%, 97%, 98%, 99%, or 100% identical to the adalimumab light chain amino acid sequence.

In some embodiments, a conjugate of this disclosure comprises an anti-TNFα antibody, wherein the anti-TNFα antibody comprises heavy chain CDR amino acid sequences of VH-CDR1, VH-CDR2, and VH-CDR3 from infliximab and light chain CDR amino acid sequences of VL-CDR1, VL-CDR2, and VL-CDR3 from infliximab. In certain embodiments, a conjugate of this disclosure comprises an anti-TNFα antibody having a VH region comprising an amino acid sequence that is at least 90%, 91%, 92%, 93%, 94%, 95%, 96%, 97%, 98%, 99%, or 100% identical to the infliximab VH amino acid sequence, and a VL region comprising an amino acid sequence that is at least 90%, 91%, 92%, 93%, 94%, 95%, 96%, 97%, 98%, 99%, or 100% identical to the infliximab VL amino acid sequence. In other embodiments, a conjugate of this disclosure comprises an anti-TNFα antibody having a heavy chain comprising an amino acid sequence that is at least 90%, 91%, 92%, 93%, 94%, 95%, 96%, 97%, 98%, 99%, or 100% identical to the infliximab heavy chain amino acid sequence, and a light chain comprising an amino acid sequence that is at least 90%, 91%, 92%, 93%, 94%, 95%, 96%, 97%, 98%, 99%, or 100% identical to the infliximab light chain amino acid sequence.

In some embodiments, a conjugate of this disclosure comprises an anti-TNFα antibody, wherein the anti-TNFα antibody comprises heavy chain CDR amino acid sequences of VH-CDR1, VH-CDR2, and VH-CDR3 from golimumab and light chain CDR amino acid sequences of VL-CDR1, VL-CDR2, and VL-CDR3 from golimumab. In certain embodiments, a conjugate of this disclosure comprises an anti-TNFα antibody having a VH region comprising an amino acid sequence that is at least 90%, 91%, 92%, 93%, 94%, 95%, 96%, 97%, 98%, 99%, or 100% identical to the golimumab VH amino acid sequence, and a VL region comprising an amino acid sequence that is at least 90%, 91%, 92%, 93%, 94%, 95%, 96%, 97%, 98%, 99%, or 100% identical to the golimumab VL amino acid sequence. In other embodiments, a conjugate of this disclosure comprises an anti-TNFα antibody having a heavy chain comprising an amino acid sequence that is at least 90%, 91%, 92%, 93%, 94%, 95%, 96%, 97%, 98%, 99%, or 100% identical to the golimumab heavy chain amino acid sequence, and a light chain comprising an amino acid sequence that is at least 90%, 91%, 92%, 93%, 94%, 95%, 96%, 97%, 98%, 99%, or 100% identical to the golimumab light chain amino acid sequence.

In some embodiments, a conjugate of this disclosure comprises an anti-TNFα antibody, wherein the anti-TNFα antibody, optionally pegylated, comprises heavy chain CDR amino acid sequences of VH-CDR1, VH-CDR2, and VH-CDR3 from certolizumab and light chain CDR amino acid sequences of VL-CDR1, VL-CDR2, and VL-CDR3 from certolizumab. In certain embodiments, a conjugate of this disclosure comprises an anti-TNFα antibody, optionally pegylated, having a VH region comprising an amino acid sequence that is at least 90%, 91%, 92%, 93%, 94%, 95%, 96%, 97%, 98%, 99%, or 100% identical to the certolizumab VH amino acid sequence, and a VL region comprising an amino acid sequence that is at least 90%, 91%, 92%, 93%, 94%, 95%, 96%, 97%, 98%, 99%, or 100% identical to the certolizumab VL amino acid sequence. In other embodiments, a conjugate of this disclosure comprises an anti-TNFα antibody, optionally pegylated, having a heavy chain comprising an amino acid sequence that is at least 90%, 91%, 92%, 93%, 94%, 95%, 96%, 97%, 98%, 99%, or 100% identical to the certolizumab heavy chain amino acid sequence, and a light chain comprising an amino acid sequence that is at least 90%, 91%, 92%, 93%, 94%, 95%, 96%, 97%, 98%, 99%, or 100% identical to the certolizumab light chain amino acid sequence (certolizumab pegol).

In some embodiments, a conjugate of this disclosure comprises an anti-TNFα fusion protein comprising from one to six etanercept TNFα-binding peptide amino acid sequences fused to an Fc region portion. In certain embodiments, one to six etanercept TNFα-binding peptides are each a TNFR ectodomain. In certain embodiments, a conjugate of this disclosure comprises an anti-TNFα fusion protein having an amino acid sequence that is at least 90%, 91%, 92%, 93%, 94%, 95%, 96%, 97%, 98%, 99%, or 100% identical to the etanercept amino acid sequence.

In certain embodiments, a binding protein conjugate of this disclosure comprises a GR agonist linked to a TNFR2 binding protein, wherein the binding protein comprises a binding domain, an antibody, an antibody construct, a fusion protein, or a targeting moiety that specifically binds to TNFR2. Such anti-TNFR2 binding domains and conjugates of the disclosure are capable of specifically binding to TNFR2 expressing cells. In certain embodiments, an anti-TNFR2 binding protein, or GR agonist conjugate thereof, of this disclosure specifically binds human TNFR2 (see, e.g., www.uniprot.org/uniprot/P20333, the sequence of which is incorporated herein by reference in its entirety) or to an epitope thereof. In further embodiments, an anti-TNFR2 binding protein, or GR agonist conjugate thereof, of this disclosure comprises an anti-TNFR2 antibody or an anti-TNFR2 fusion protein.

In certain embodiments, a binding protein conjugate of this disclosure comprises a GR agonist linked to an APRIL binding protein, wherein the binding protein comprises a binding domain, an antibody, an antibody construct, a fusion protein, or a targeting moiety that specifically binds to APRIL. Such anti-APRIL binding domains and conjugates of this disclosure are capable of specifically binding to APRIL expressing cells. In certain embodiments, an anti-APRIL binding protein, or GR agonist conjugate thereof, of this disclosure specifically binds human APRIL (see, e.g., www.uniprot.org/uniprot/O75888, the sequence of which is incorporated herein by reference in its entirety) or to an epitope thereof. In further embodiments, an anti-APRIL binding protein, or GR agonist conjugate thereof, of this disclosure comprises an anti-APRIL antibody or an anti-APRIL fusion protein.

In some embodiments, a conjugate of this disclosure comprises an anti-APRIL antibody, wherein the anti-APRIL antibody comprises heavy chain CDR amino acid sequences of VH-CDR1, VH-CDR2, and VH-CDR3 from sibeprenlimab and light chain CDR amino acid sequences of VL-CDR1, VL-CDR2, and VL-CDR3 from sibeprenlimab. In certain embodiments, a conjugate of this disclosure comprises an anti-APRIL antibody having a VH region comprising an amino acid sequence that is at least 90%, 91%, 92%, 93%, 94%, 95%, 96%, 97%, 98%, 99%, or 100% identical to the sibeprenlimab VH amino acid sequence, and a VL region comprising an amino acid sequence that is at least 90%, 91%, 92%, 93%, 94%, 95%, 96%, 97%, 98%, 99%, or 100% identical to the sibeprenlimab VL amino acid sequence. In other embodiments, a conjugate of this disclosure comprises an anti-APRIL antibody having a heavy chain comprising an amino acid sequence that is at least 90%, 91%, 92%, 93%, 94%, 95%, 96%, 97%, 98%, 99%, or 100% identical to the sibeprenlimab heavy chain amino acid sequence, and a light chain comprising an amino acid sequence that is at least 90%, 91%, 92%, 93%, 94%, 95%, 96%, 97%, 98%, 99%, or 100% identical to the sibeprenlimab light chain amino acid sequence.

In some embodiments, a conjugate of this disclosure comprises an anti-APRIL antibody, wherein the anti-APRIL antibody comprises heavy chain CDR amino acid sequences of VH-CDR1, VH-CDR2, and VH-CDR3 from BION-1301 and light chain CDR amino acid sequences of VL-CDR1, VL-CDR2, and VL-CDR3 from BION-1301. In certain embodiments, a conjugate of this disclosure comprises an anti-APRIL antibody having a VH region comprising an amino acid sequence that is at least 90%, 91%, 92%, 93%, 94%, 95%, 96%, 97%, 98%, 99%, or 100% identical to the BION-1301 VH amino acid sequence, and a VL region comprising an amino acid sequence that is at least 90%, 91%, 92%, 93%, 94%, 95%, 96%, 97%, 98%, 99%, or 100% identical to the BION-1301 VL amino acid sequence. In other embodiments, a conjugate of this disclosure comprises an anti-APRIL antibody having a heavy chain comprising an amino acid sequence that is at least 90%, 91%, 92%, 93%, 94%, 95%, 96%, 97%, 98%, 99%, or 100% identical to the BION-1301 heavy chain amino acid sequence, and a light chain comprising an amino acid sequence that is at least 90%, 91%, 92%, 93%, 94%, 95%, 96%, 97%, 98%, 99%, or 100% identical to the BION-1301 light chain amino acid sequence.

In any of the aforementioned embodiments, a binding protein or a GR agonist conjugate thereof of this disclosure specifically binds to an epitope that has a continuous amino acid sequence or a discontinuous amino acid sequence. In any of the aforementioned embodiments, a binding protein or a conjugate thereof of this disclosure specifically binds to a conformational epitope.

D. Nucleic Acids, Vectors, and Host Cells

The instant disclosure provides an isolated nucleic acid that encodes a fusion protein (e.g., an anti-target fusion protein as provided herein) or an antibody (e.g., an anti-target antibody as provided herein), or a target or antigen binding fragment thereof, of this disclosure. In some embodiments, a nucleic acid encoding a fusion protein or antibody of this disclosure, or an antigen binding fragment thereof, is codon optimized to enhance or maximize expression in certain types of cells (e.g., Scholten et al., Clin. Immunol. 119: 135-145, 2006). As used throughout the disclosure, a “codon optimized” polynucleotide is a heterologous polypeptide having codons modified with silent mutations corresponding to the abundances of host cell tRNA levels.

In some embodiments, a nucleic acid molecule encodes a fusion protein (e.g., an anti-target fusion protein as provided herein) or antibody (e.g., an anti-target antibody as provided herein), or a target or antigen binding fragment thereof (e.g., an antibody heavy and light chains, or an antibody binding domain comprising VH and VL binding regions) as disclosed herein wherein two or more chains or regions are separated by a cleavage site. In further embodiments, a cleavage site is a self-cleaving amino acid sequence comprising a 2A peptide from porcine teschovirus-1 (P2A), equine rhinitis A virus (E2A), Thosea asigna virus (T2A), foot-and-mouth disease virus (F2A), or any combination thereof (see, e.g., Kim et al., PLOS One 6:e18556, 2011, which 2A nucleic acid and amino acid sequences are incorporated herein by reference in their entirety). In some embodiments, a nucleic acid molecule comprising a nucleotide sequence encoding a heavy chain of a fusion protein or an antibody (e.g., an anti-target antibody as provided herein) or its variable region is provided. In some embodiments, a nucleic acid molecule comprising a nucleotide sequence encoding a light chain of a fusion protein or an antibody (e.g., an anti-target antibody as provided herein) or its variable region is provided.

In certain embodiments, an expression construct comprising a nucleic acid encoding a fusion protein (e.g., an anti-target fusion protein as provided herein) or an antibody (e.g., an anti-Target antibody as provided herein), or a target or antigen binding fragment thereof, of the disclosure is provided. In some embodiments, a nucleic acid may be operably linked to an expression control sequence. As used herein, “expression construct” refers to a DNA construct containing a nucleic acid molecule that is operably linked to a suitable control sequence capable of effecting the expression of the nucleic acid molecule in a suitable host. An expression construct may be present in a vector (e.g., a bacterial vector, a viral vector) or may be integrated into a genome. The term “operably linked” refers to the association of two or more nucleic acids on a single polynucleotide fragment so that the function of one is affected by the other. For example, a promoter is operably linked with a coding sequence when it can affect the expression of that coding sequence (i.e., the coding sequence is under the transcriptional control of the promoter). The term “expression control sequence” (also called a regulatory sequence) refers to nucleic acid sequences that effect the expression and processing of coding sequences to which they are operably linked. For example, expression control sequences may include transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In some embodiments, a nucleic acid or an expression construct encoding a fusion protein (e.g., an anti-target fusion protein as provided herein) or an antibody (e.g., an anti-Target antibody as provided herein), or a target or antigen binding fragment thereof, is present in a vector. A “vector” is a nucleic acid molecule that can transport another nucleic acid. Vectors may be, for example, plasmids, cosmids, viruses, an RNA vector or a linear or circular DNA or RNA molecule that may include chromosomal, non-chromosomal, semi-synthetic or synthetic nucleic acids. Exemplary vectors are those capable of autonomous replication (episomal vector) or expression of nucleic acids to which they are linked (expression vectors).

Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). In some embodiments, a vector is a plasmid. In some other embodiments, a vector is a viral vector. In some such embodiments, the viral vector is a lentiviral vector or a γ-retroviral vector.

In some embodiments, the instant disclosure provides an isolated host cell comprising a nucleic acid, expression construct, or vector encoding a fusion protein (e.g., an anti-target fusion protein as provided herein) or an antibody (e.g., an anti-target antibody as provided herein), or a target or antigen binding fragment thereof, of the disclosure. As used herein, the term “host” refers to a cell or microorganism targeted for genetic modification with a heterologous or exogenous nucleic acid molecule to produce a polypeptide of interest (e.g., a fusion protein or its target binding domain an antibody or its antigen-binding fragment). In some embodiments, a host cell may optionally already possess or be modified to include other genetic modifications that confer desired properties related or unrelated to biosynthesis of the heterologous or exogenous protein (e.g., inclusion of a detectable marker). More than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two more exogenous nucleic acid molecules can be introduced as a single nucleic acid molecule (e.g., on a single vector), on separate vectors, integrated into the host chromosome at a single site or multiple sites. The number of referenced heterologous nucleic acid molecules or protein activities refers to the number of encoding nucleic acid molecules or the number of protein activities, not the number of separate nucleic acid molecules introduced into a host cell.

E. Linkers and Linker-Payloads

A compound of the present disclosure, such as a compound of Formula I or II, may be bound to a linker, e.g., a peptide containing linker or cleavable linker. In some embodiments, a linker is also bound to a polypeptide comprising a binding domain (e.g., a fusion protein), an antibody, an antibody construct, or a targeting moiety that specifically binds a target, thereby forming a conjugate comprising the polypeptide and the compound. Linkers of the conjugates may not affect the binding of active portions of a conjugate, e.g., the antigen binding domains, Fc region or domains, target binding domain, antibody, targeting moiety, or the like, to a target, which can be a cognate binding partner, such as an antigen. A conjugate can comprise multiple linkers, each having one or more compounds attached. The multiple linkers can be the same linker or different linkers contained on a single conjugate or on separate conjugates.

In some embodiments of this disclosure, a linker connects one or more compound(s) of the disclosure to a polypeptide comprising a target binding domain (e.g., a fusion protein, or an antibody or an antigen-binding fragment thereof) by forming a covalent linkage to the compound at one location and a covalent linkage to the polypeptide comprising the binding domain at another location. The covalent linkages can be formed by reaction between functional groups on the linker and functional groups on the compound and on the polypeptide comprising the binding domain. As used herein, the term “linker” can include (i) unattached forms of the linker that can include a functional group capable of covalently attaching the linker to a compound and a functional group capable of covalently attaching the linker to the polypeptide comprising the binding domain or binding fragment thereof (e.g., an antibody or an antigen-binding fragment thereof); (ii) partially attached forms of the linker that can include a functional group capable of covalently attaching the linker to polypeptide comprising the binding domain or binding fragment thereof (e.g., an antibody or an antigen-binding fragment thereof), and that can be covalently attached to a compound, or vice versa; and (iii) fully attached forms of the linker that can be covalently attached to both an GR agonist and to the polypeptide comprising the binding domain or binding fragment thereof (e.g., an antibody or an antigen-binding fragment thereof). In some embodiments, a functional group on a linker and covalent linkages formed between the linker and a polypeptide comprising the binding domain, can be specifically illustrated as Rx and Rx′, respectively.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave or immolate or otherwise breakdown specifically or non-specifically inside cells. A cleavable linker can be sensitive to enzymes. A cleavable linker can be cleaved by enzymes such as proteases.

A cleavable linker can include a valine-citrulline (Val-Cit) peptide, a valine-alanine (Val-Ala) peptide, a phenylalanine-lysine (Phe-Lys) or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a maleimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)— linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain a maleimide(s) linked to polyethylene glycol molecules in which the polyethylene glycol can allow for more linker flexibility or can be used lengthen the linker.

A linker can be a (maleimidocaproyl)-(valine-alanine)-(para-aminobenzyloxycarbonyl) linker. A linker can be a (maleimidocaproyl)-(valine-citrulline)-(para-aminobenzyloxycarbonyl) linker. A linker can be a (maleimidocaproyl)-(phenylalanine-lysine)-(para-aminobenzyloxycarbonyl) linker. A linker can be a linker suitable for attachment to an engineered cysteine (THIOMAB). A THIOMAB linker can be a (maleimidocaproyl)-(valine-citrulline)-(para-aminobenzyloxycarbonyl)-linker.

A linker can also contain segments of alkylene, alkenylene, alkynylene, polyether, polyester, polyamide, polyamino acids, peptides, polypeptides, cleavable peptides, or aminobenzyl-carbamates. A linker can contain a maleimide at one end and an N-hydroxysuccinimidyl ester at the other end. A linker can contain a lysine with an N-terminal amine acetylated, and a valine-citrulline, valine-alanine or phenylalanine-lysine cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG recognition motif to an N-terminal GGG motif to regenerate a native amide bond. The linker created can therefore link to a moiety attached to the LPXTG recognition motif with a moiety attached to the N-terminal GGG motif. A linker can be a link created between an unnatural amino acid on one moiety reacting with oxime bond that was formed by modifying a ketone group with an alkoxyamine on another moiety. A moiety can be part of a conjugate. A moiety can be part of an antibody. A moiety can be part of a GR agonist. A moiety can be part of a binding domain. A linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In some embodiments of conjugates of the disclosure, a compound, or salt, stereoisomer, solvate or prodrug thereof is linked to the polypeptide comprising a target binding domain (e.g., a fusion protein, or an antibody or an antigen-binding fragment thereof) by way of a linker(s), also referred to herein as L or L³. L, as used herein, may be selected from any of the linker moieties discussed herein. The linker linking the compound or salt thereof to the polypeptide of the binding domain of a conjugate may be short, long, hydrophobic, hydrophilic, flexible, or rigid, or may be composed of segments that each independently have one or more of the above-mentioned properties such that the linker may include segments having different properties. The linkers may be polyvalent such that they covalently link more than one compound or salt to a single site on the polypeptide comprising the binding domain, or fragment thereof, or monovalent such that covalently they link a single compound or salt to a single site on the binding domain, or fragment thereof.

A linker can be polyvalent such that it covalently links more than one compound of the present disclosure to a single site on the polypeptide comprising the binding domain (e.g., a fusion protein, or an antibody or an antigen-binding fragment thereof) or monovalent such that it covalently links a single compound to a single site on the binding domain, or fragment thereof.

In some embodiments for a compound of the present disclosure, such as a compound of Formula I or II, or pharmaceutically acceptable isomer, tautomer, racemate, hydrate, solvate, isotope, or salt thereof, the compound may further comprise a linker (L), which results in a linker-payload. The linker may be covalently bound to any position, valence permitting, on a compound, or a pharmaceutically acceptable isomer, tautomer, racemate, hydrate, solvate, isotope, or salt thereof. For example, the linker may be bound to a nitrogen atom, e.g., an amine, or oxygen atom, e.g., a hydroxyl, a sulfur, e.g., a thiol, of a compound, or pharmaceutically acceptable isomer, tautomer, racemate, hydrate, solvate, isotope, or salt thereof. The linker may comprise a reactive moiety, e.g., an electrophile that can react to form a covalent bond with a reactive moiety of a binding protein of this disclosure, e.g., a lysine, serine, threonine, cysteine, tyrosine, aspartic acid, glutamine, a non-natural amino acid residue, or glutamic acid residue. In some embodiments, a compound, or pharmaceutically acceptable isomer, tautomer, racemate, hydrate, solvate, isotope, or salt thereof, may be covalently bound through the linker to a binding domain, such as an antibody, an antibody construct, or a targeting moiety.

In the conjugates, a compound of the present disclosure, such as a compound of Formula I or II, or a pharmaceutically acceptable isomer, tautomer, racemate, hydrate, solvate, isotope, or salt thereof, is linked to a polypeptide comprising a binding domain (e.g., fusion protein), such as an antibody, an antibody construct, or a targeting moiety by way of a linker(s), also referred to herein as L, as used herein, may be selected from any of the linker moieties discussed herein. The linker linking the compound or salt to a polypeptide comprising a binding domain, such as an antibody, an antibody construct, or a targeting moiety of a conjugate may be short, long, hydrophobic, hydrophilic, flexible, or rigid, or may be composed of segments that each independently have one or more of the above-mentioned properties, such that the linker may include segments having different properties. The linkers may be polyvalent such that they covalently link more than one compound or salt to a single site on a polypeptide comprising a binding domain, such as an antibody, an antibody construct, or a targeting moiety, or monovalent, such that covalently they link a single compound or salt to a single site on the polypeptide comprising a binding domain, such as an antibody, an antibody construct, or a targeting moiety.

Linkers of the disclosure (L) may have from about 10 to about 500 atoms in a linker, such as from about 10 to about 400 atoms, such as about 10 to about 300 atoms in a linker. In some embodiments, linkers of the disclosure have from about 30 to about 400 atoms, such as from about 30 to about 300 atoms in the linker.

Linkers of the disclosure may link a compound of the present disclosure, such as a compound of Formula I or II, or pharmaceutically acceptable isomer, tautomer, racemate, hydrate, solvate, isotope, or salt thereof, to a binding protein of this disclosure by covalent linkages between the linker and the binding protein of this disclosure, and the GR agonist compound, to form a conjugate.

As used throughout the disclosure, the expression “linker” is intended to include (i) unconjugated forms of the linker that include a functional group capable of covalently linking the linker to a compound of the present disclosure, or pharmaceutically acceptable isomer, tautomer, racemate, hydrate, solvate, isotope, or salt thereof, and a functional group capable of covalently linking the linker to a binding protein of this disclosure; (ii) partially conjugated forms of the linker that include a functional group capable of covalently linking the linker to the polypeptide, and that is covalently linked to at least one compound of the present disclosure, or pharmaceutically acceptable isomer, tautomer, racemate, hydrate, solvate, isotope or salt thereof, or vice versa; and (iii) fully conjugated forms of the linker that is covalently linked to both a compound of the present disclosure, or pharmaceutically acceptable isomer, tautomer, racemate, hydrate, solvate, isotope, or salt thereof, and a binding protein of this disclosure. Some embodiments pertain to a conjugate formed by contacting a binding protein of this disclosure that binds a cell surface receptor or antigen expressed on a target cell with a linker-compound of this disclosure under conditions in which the linker-compound covalently links to a binding protein of this disclosure. Further embodiments pertain to a method of making a conjugate formed by contacting a linker-compound under conditions in which the linker-compound covalently links to a binding protein of this disclosure.

In some embodiments, a compound of the present disclosure, or pharmaceutically acceptable isomer, tautomer, racemate, hydrate, solvate, isotope, or salt thereof, is covalently bound to a linker (L) to form a linker-payload (L-P or “linker payload”). The linker may be covalently bound to any position of the compound, valence permitting. The linker may comprise a reactive moiety, e.g., an electrophile that can react to form a covalent bond with a moiety of a binding protein of this disclosure, such as, for example, a lysine, serine, threonine, cysteine, tyrosine, aspartic acid, glutamine, a non-natural amino acid residue, or glutamic acid residue. In some embodiments, a linker-payload, comprising a GR agonist compound or salt of a GR agonist compound and a linker, L, is covalently bound through the linker to a binding protein of this disclosure.

In some embodiments, a linker-payload, comprising a compound or salt thereof of this disclosure and a linker, L, is covalently bound through L to an antibody. In further embodiments, a linker-payload, comprising a compound or salt thereof of this disclosure and a linker, L, is covalently bound through L to an antigen-binding fragment of an antibody. In still further embodiments, a linker-payload, comprising a compound or salt thereof of this disclosure and a linker, L, is covalently bound through L to a fusion protein. In some embodiments, for a linker-payload comprising a compound of Formula I or II, or pharmaceutically acceptable isomer, tautomer, racemate, hydrate, solvate, isotope, or salt thereof, L is a non-cleavable linker. Alternatively, in some embodiments, for a linker-payload comprising a compound of Formula I or II, or pharmaceutically acceptable isomer, tautomer, racemate, hydrate, solvate, isotope, or salt thereof, L is a cleavable linker, such as a linker cleavable by a lysosomal enzyme. In some embodiments, the polypeptide may further comprise a second antigen or target binding domain.

In certain embodiments, a GR agonist compound of this disclosure is covalently attached to an antibody. In further embodiments, a GR agonist compound of this disclosure is covalently attached to an antigen-binding fragment of an antibody. In other embodiments, a compound of this disclosure is covalently attached to a fusion protein. In some embodiments, a binding protein of this disclosure further comprises a second target binding domain.

Category I Linkers

Exemplary polyvalent linkers that may be used to link compounds of this disclosure to a polypeptide comprising a target binding domain, such as an antibody construct, are described. For example, Fleximer® linker technology has the potential to enable high-DAR conjugates with good physicochemical properties. As shown below, the Fleximer® linker technology is based on incorporating drug molecules into a solubilizing poly-acetal backbone via a sequence of ester bonds:

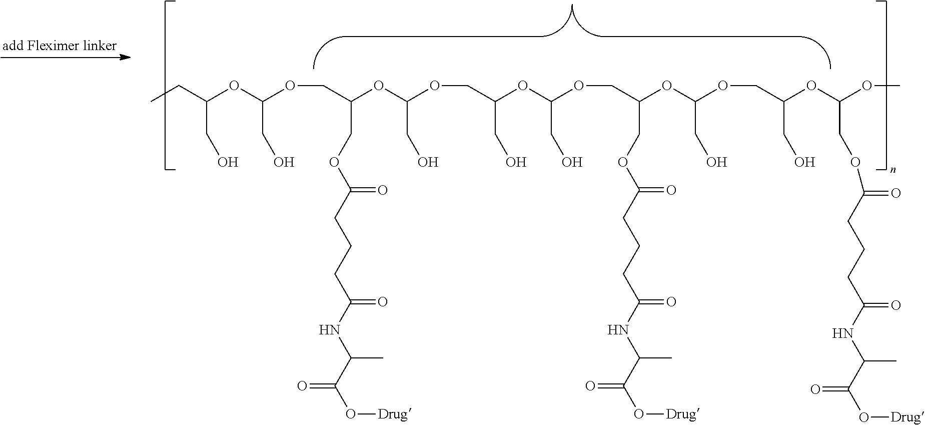

The methodology renders highly loaded conjugates (DAR up to 20) whilst maintaining good physicochemical properties. This methodology can be utilized with a GR agonist compound as shown in the scheme below, where Drug′ refers to the GR agonist compound. To utilize the Fleximer® linker technology depicted in the scheme above, an aliphatic alcohol can be present or introduced into the GR agonist compound. The alcohol moiety is then attached to an alanine moiety, which is then synthetically incorporated into the Fleximer® linker. Liposomal processing of the conjugate in vitro releases the parent alcohol-containing drug.

In some embodiments, a moiety, construct, or conjugate of the disclosure includes the symbol [symbol] , which indicates the point of attachment, e.g., the point of attachment of a chemical or functional moiety to the compound, the point of attachment of a linker to a compound of the disclosure, or the point of attachment of a linker to a polypeptide comprising the binding domain (e.g., a fusion protein, or an antibody or an antigen-binding fragment thereof).

By way of example and not limitation, some cleavable and non-cleavable linkers that may be included in the conjugates are described below, in addition to any other of the disclosure.

Sulfamide linkers may be used to link many compounds of the present disclosure to an antibody construct. Sulfamide linkers of the disclosure include, e.g., U.S. Patent Publication Number 2019/0038765, the linkers of which are incorporated by reference herein.

Cleavable linkers can be cleavable in vitro, in vivo, or both. Cleavable linkers can include chemically or enzymatically unstable or degradable linkages. Cleavable linkers can rely on processes inside the cell to liberate a compound of Categories A to K such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

In some embodiments, L is a linker comprising a reactive moiety. In some embodiments, for a linker-payload comprising a compound of the present disclosure or pharmaceutically acceptable isomer, tautomer, racemate, hydrate, solvate, isotope, or salt thereof, -L is represented by the formula:

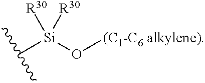

In some embodiments, -L is represented by the formula:

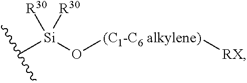

wherein each R³⁰ is independently selected from optionally substituted C₁-C₆ alkyl and optionally substituted phenyl, and RX is the reactive moiety. RX may comprise a leaving group. RX may be a maleimide. L may be further covalently bound to a binding protein of this disclosure. In some embodiments, -L- is represented by the formula:

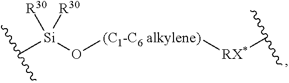

wherein RX* is a bond, a succinimide moiety, or a hydrolyzed succinimide moiety bound to a residue of a binding protein of this disclosure, wherein

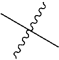

on RX* represents the point of attachment to a residue of the polypeptide; and each R³⁰ is independently selected from optionally substituted C₁-C₆ alkyl and optionally substituted phenyl.

In some embodiments, for a linker-payload comprising a compound of the present disclosure or pharmaceutically acceptable isomer, tautomer, racemate, hydrate, solvate, isotope, or salt thereof, and linker L; L comprises a methylene carbamate unit.

In some embodiments, for a linker-payload (L-P) comprising a compound of the present disclosure or pharmaceutically acceptable isomer, tautomer, racemate, hydrate, solvate, isotope, or salt thereof, and linker L-RX*; the L-P is part of a conjugate and RX* comprises a hydrolyzed succinimide moiety and is bound to a cysteine residue of a polypeptide comprising a binding domain.

By way of example and not limitation, some cleavable and non-cleavable linkers that may be included in the conjugates are described below, in addition to any others of the disclosure.

A linker can contain a chemically labile group such as hydrazone or disulfide groups. Linkers comprising chemically labile groups can exploit differential properties between the plasma and some cytoplasmic compartments. The intracellular conditions that can facilitate release of a compound of the present disclosure, or pharmaceutically acceptable isomer, tautomer, racemate, hydrate, solvate, isotope, or salt thereof for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a compound of the present disclosure once the conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

In some embodiments, for a linker-payload comprising a compound of the present disclosure, or pharmaceutically acceptable isomer, tautomer, racemate, hydrate, solvate, isotope, or salt thereof, and a linker L, -L comprises a hydrazone moiety. For example, L may be selected from:

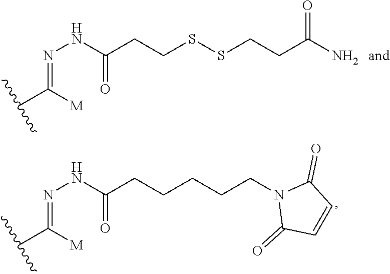

wherein M is selected from C₁-C₆ alkyl, aryl, and —O—C₁-C₆ alkyl.

Hydrazone-containing linkers can contain additional cleavage sites, such as additional acid-labile cleavage sites or enzymatically labile cleavage sites. Conjugates including exemplary hydrazone-containing linkers can include, for example, the following structures:

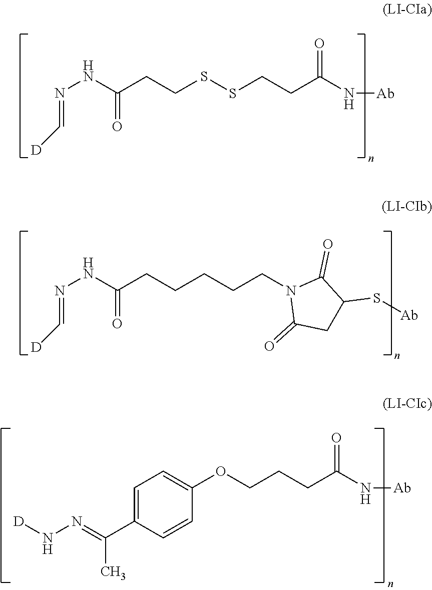

wherein D is a compound or salt of the present disclosure, and Ab is a binding protein of this disclosure, respectively, and n represents the number of compound-bound linkers (LP) bound to the polypeptide. In certain linkers, such as linker (Ia), the linker can comprise two cleavable groups, a disulfide, and a hydrazone moiety. For such linkers, effective release of the unmodified free compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release a compound of the present disclosure, or pharmaceutically acceptable isomer, tautomer, racemate, hydrate, solvate, isotope, or salt thereof, upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing a compound of the present disclosure, or pharmaceutically acceptable isomer, tautomer, racemate, hydrate, solvate, isotope, or salt thereof; in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Conjugates comprising a compound of the present disclosure, or pharmaceutically acceptable isomer, tautomer, racemate, hydrate, solvate, isotope, or salt thereof, and including exemplary disulfide-containing linkers can include the following structures:

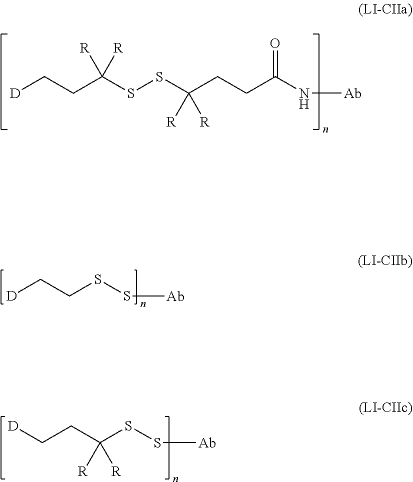

wherein D is a compound of the present disclosure, or pharmaceutically acceptable isomer, tautomer, racemate, hydrate, solvate, isotope, or salt thereof, and Ab is a binding protein of this disclosure, n represents the number of compounds bound to linkers (L) bound to the polypeptide and R is independently selected at each occurrence from, for example, hydrogen or alkyl. Increasing steric hindrance adjacent to the disulfide bond can increase the stability of the linker.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a compound of the present disclosure, or pharmaceutically acceptable isomer, tautomer, racemate, hydrate, solvate, isotope, or salt thereof, from a binding protein of this disclosure can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly, Ala-Leu-Ala-Leu or dipeptides such as Val-Cit, Val-Ala, and Phe-Lys. Dipeptides can have lower hydrophobicity compared to longer peptides.

A variety of dipeptide-based cleavable linkers can be used with a binding protein of this disclosure to form conjugates of a compound of the present disclosure, or pharmaceutically acceptable isomer, tautomer, racemate, hydrate, solvate, isotope, or salt thereof, of the disclosure.

Enzymatically cleavable linkers can include a self-immolative spacer to spatially separate a compound of the present disclosure, or pharmaceutically acceptable isomer, tautomer, racemate, hydrate, solvate, isotope, or salt thereof, from the site of enzymatic cleavage. The direct attachment of a compound of the present disclosure, or pharmaceutically acceptable isomer, tautomer, racemate, hydrate, solvate, isotope, or salt thereof, to a peptide linker can result in proteolytic release of a compound of the present disclosure, or pharmaceutically acceptable isomer, tautomer, racemate, hydrate, solvate, isotope, or salt thereof, or of an amino acid adduct of a compound of the present disclosure, or pharmaceutically acceptable isomer, tautomer, racemate, hydrate, solvate, isotope, or salt thereof, thereby impairing its activity. The use of a self-immolative spacer can allow for the elimination of the fully active, chemically unmodified compound of the present disclosure, or pharmaceutically acceptable isomer, tautomer, racemate, hydrate, solvate, isotope, or salt thereof, upon amide bond hydrolysis.

One self-immolative spacer can be a bifunctional para-aminobenzyl alcohol (PABA) group, which can link to a peptide through an amino group, forming an amide bond, while an amine containing compound of the present disclosure, or pharmaceutically acceptable isomer, tautomer, racemate, hydrate, solvate, isotope, or salt thereof, can be attached through carbamate functionalities to the benzylic hydroxyl group of the linker (to give a p-amidobenzylcarbamate, PABC). The resulting pro-compound can be activated upon protease-mediated cleavage, leading to a 1,6-elimination reaction releasing the unmodified compound of the present disclosure, or pharmaceutically acceptable isomer, tautomer, racemate, hydrate, solvate, isotope, or salt thereof, carbon dioxide, and remnants of the linker group.

In some embodiments, the compound of Categories A to K, or pharmaceutically acceptable isomer, tautomer, racemate, hydrate, solvate, isotope, or salt thereof, is bound to an LI-type linker, which linker may comprise a reactive group capable of forming a covalent bond with a reactive group (e.g., —SH or NH₂) on a binding protein of this disclosure, or the linker may already be covalently linked to a binding protein of this disclosure. In some embodiments, the linker has the following structure:

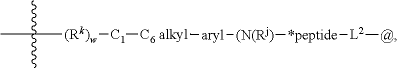

wherein:

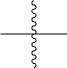

indicates the covalent bond R′;
R^{k} is

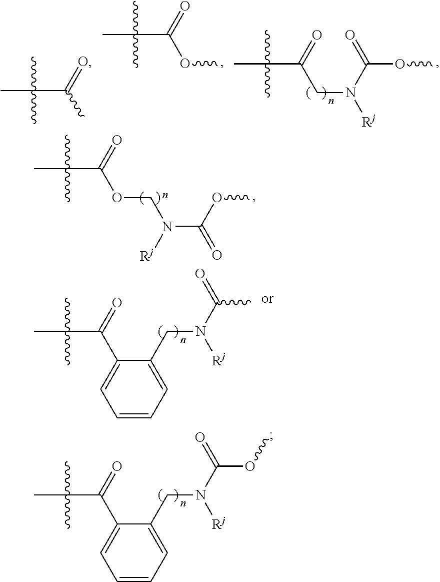

w is 0 or 1;
R^{j} is, at each occurrence, independently H, or C₁-C₂₀ alkyl;
* is the C-terminal of the peptide;
L² is absent, —C₁-C₁₂ alkyl, —C₁-C₁₂ heteroalkyl, —C(═O)C₁-C₁₂ alkyl or —C(O)C₁-C₁₂ heteroalkyl;
@ is a reactive group capable of forming a covalent bond with a binding protein of this disclosure, or is a group comprising a covalent bond to a residue of a binding protein of this disclosure;
[symbol] is a covalent attachment to the C₁-C₆ alkyl; and
n is an integer from 1 to 6.

The following scheme depicts the fragmentation of p-amidobenzyl carbamate and release of a compound of the present disclosure, or pharmaceutically acceptable isomer, tautomer, racemate, hydrate, solvate, isotope, or salt thereof:

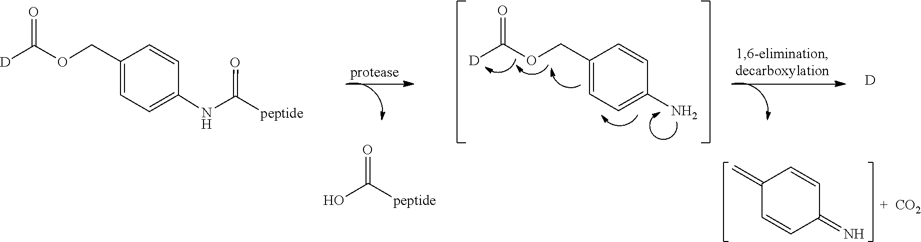

wherein D represents the drug or payload having the structure of a compound of the present disclosure, or pharmaceutically acceptable isomer, tautomer, racemate, hydrate, solvate, isotope, or salt thereof, wherein one hydrogen atom has been replaced with a bond to the —C(═O)O— group. In some embodiments of the above scheme, D is bound to the —C(═O)O— group via a N atom.

In some other embodiments, D is bound to the L1-type linker via a S atom on D. In such embodiments, the following scheme depicts the fragmentation of p-amidobenzyl carbamate and release of a compound of the present disclosure, or pharmaceutically acceptable isomer, tautomer, racemate, hydrate, solvate, isotope, or salt thereof:

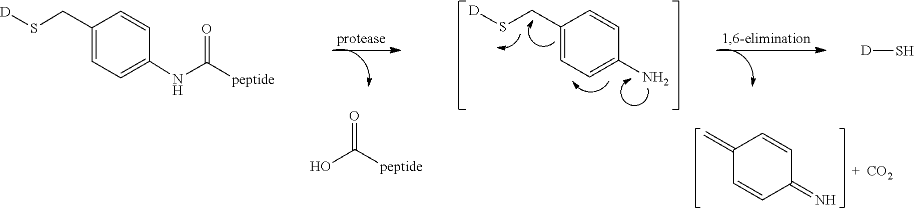

wherein D-S represents the drug or payload having the structure of a compound of the present disclosure, or pharmaceutically acceptable isomer, tautomer, racemate, hydrate, solvate, isotope,
or salt thereof, wherein one hydrogen atom has been replaced with a bond to the benzylic carbon atom and D-SH represents the released GR agonist.

In some embodiments, for a linker-payload comprising a compound of the present disclosure, or pharmaceutically acceptable isomer, tautomer, racemate, hydrate, solvate, isotope, or salt thereof, the linker (i.e., L) is represented by the formula:

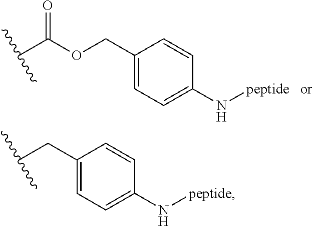

wherein peptide comprises from one to ten amino acids, and [symbol] represents the point of attachment to the compound (payload).

In some embodiments, for a linker-payload comprising a compound of the present disclosure, or pharmaceutically acceptable isomer, tautomer, racemate, hydrate, solvate, isotope, or salt thereof, -L is represented by the formula:

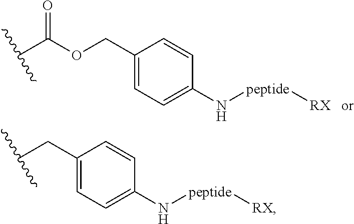

wherein peptide comprises from one to ten amino acids and RX is a reactive moiety, and [symbol] represents the point of attachment to the compound (payload).

In some embodiments, for a linker-payload comprising a compound of the present disclosure, or pharmaceutically acceptable isomer, tautomer, racemate, hydrate, solvate, isotope, or salt thereof, -L is represented by the formula:

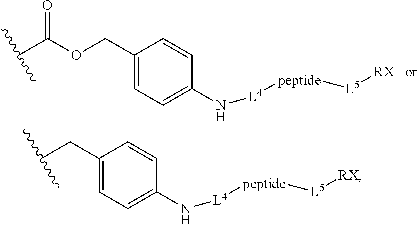

wherein peptide comprises from one to ten amino acids, L⁴ is the C-terminus of the peptide and L⁵ is selected from a bond, alkylene and heteroalkylene, wherein L⁵ is optionally substituted with one or more groups independently selected from R³², RX is a reactive moiety; and R³² is independently selected at each occurrence from halogen, —OH, —CN, —O-alkyl, —SH, ═O, ═S, —S(O)₂OH, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —S(O)₂OH, —NH₂, and —NO₂. The reactive moiety may be selected from an electrophile, e.g., an αβ-unsaturated carbonyl, such as a maleimide, and a leaving group. In some embodiments, RX comprises a leaving group. In some embodiments, RX is a maleimide.

In some embodiments, for a linker-payload comprising a GR agonist compound, or pharmaceutically acceptable isomer, tautomer, racemate, hydrate, solvate, isotope, or salt thereof, the L-P is part of a conjugate and -L is represented by the formula:

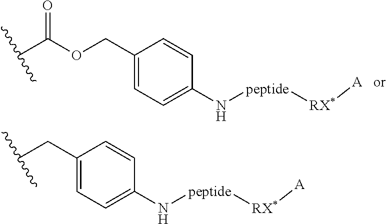

wherein A is a binding protein of this disclosure, RX* is a reactive moiety that has reacted with a moiety on the polypeptide to form a conjugate, peptide comprises from one to ten amino acids, and [symbol] represents the point of attachment to the compound (payload).

In further embodiments, L-P is part of a conjugate and -L- is represented by the formula:

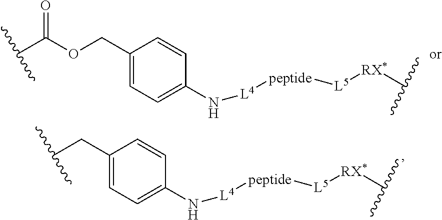

wherein peptide comprises from one to ten amino acids, L⁴ is the C-terminus of the peptide and L⁵ is selected from a bond, an alkylene and a heteroalkylene, each of which is optionally substituted with one or more groups independently selected from R¹²; [symbol] on the left represents the point of attachment to the compound (payload), RX* is a bond, a succinimide moiety, or a hydrolyzed succinimide moiety attached at the [symbol] on the right to a residue of a polypeptide comprising a target binding domain, such as an antibody or a fusion protein.

In some embodiments, L-P is part of a conjugate and -L- is represented by the formula:

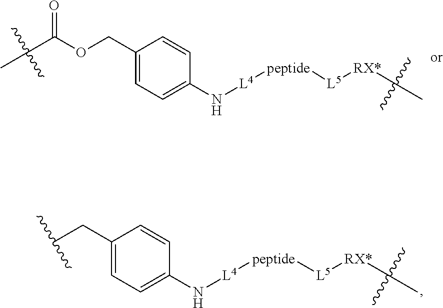

wherein peptide comprises from one to ten amino acids, L⁴ represents the C-terminus of the peptide and L⁵ is selected from a bond, alkylene and heteroalkylene, wherein L⁵ is optionally substituted with one or more groups independently selected from R³²; RX* is a bond, a succinimide moiety, or a hydrolyzed succinimide moiety bound to a residue of a binding protein of this disclosure, wherein

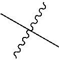

on RX* represents the point of attachment to the residue of the binding protein of this disclosure; and R³² is independently selected at each occurrence from halogen, —OH, —CN, —O-alkyl, —SH, ═O, ═S, —S(O)₂OH, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —S(O)₂OH, —NH₂, and —NO₂. In some embodiments, the peptide of L comprises Val-Cit or Val-Ala.

In some embodiments, -L is

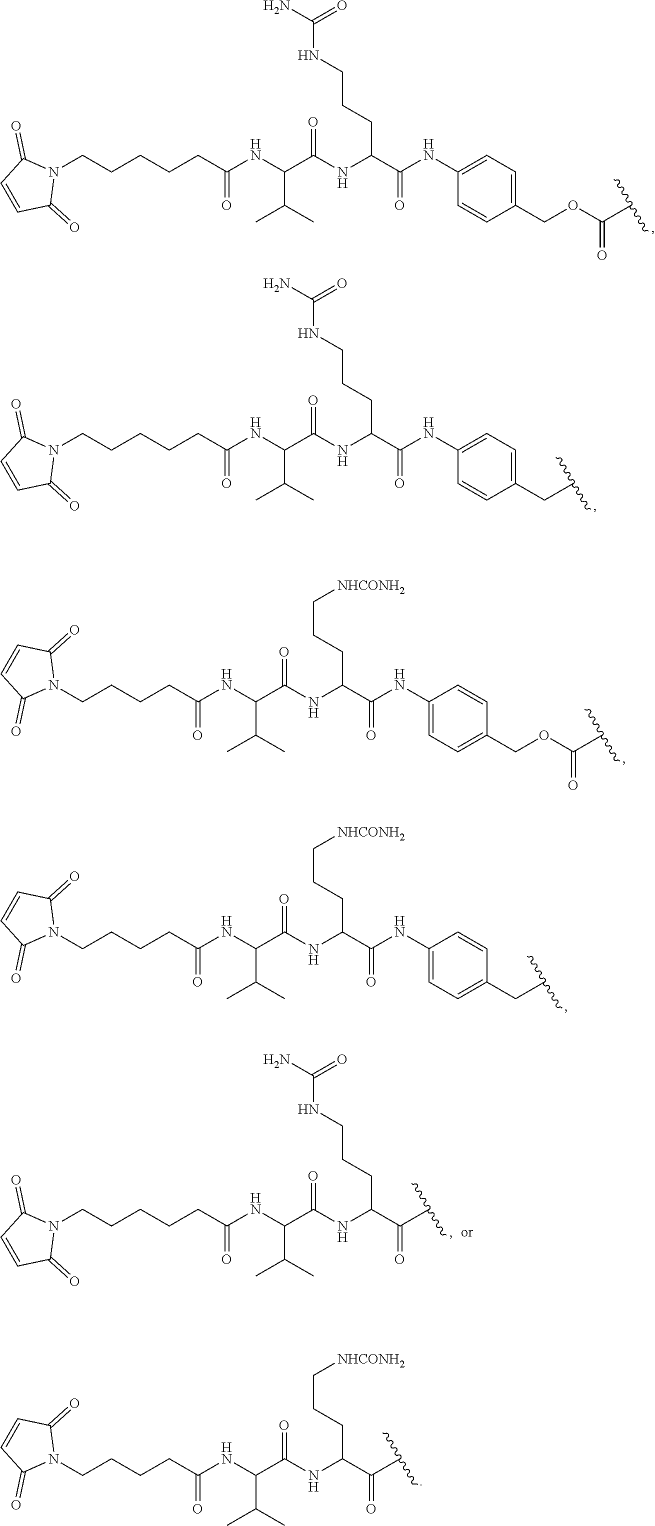

Heterocyclic variants of this self-immolative group may also be used.

The enzymatically cleavable linker can be a ß-glucuronic acid-based linker. Facile release of a compound of the present disclosure, or pharmaceutically acceptable isomer, tautomer, racemate, hydrate, solvate, isotope, or salt thereof, can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of a polypeptide conjugate of a compound of the present disclosure, or pharmaceutically acceptable isomer, tautomer, racemate, hydrate, solvate, isotope, or salt thereof, to undergo aggregation due to the hydrophilic nature of ß-glucuronides. In some embodiments, ß-glucuronic acid-based linkers can link a binding protein of this disclosure to a hydrophobic compound of the present disclosure, or pharmaceutically acceptable isomer, tautomer, racemate, hydrate, solvate, isotope, or salt thereof.

The following scheme depicts the release of a compound of the present disclosure, or pharmaceutically acceptable isomer, tautomer, racemate, hydrate, solvate, isotope, or salt thereof, (D) from a conjugate of a compound of the present disclosure, or pharmaceutically acceptable isomer, tautomer, racemate, hydrate, solvate, isotope, or salt thereof, containing a ß-glucuronic acid-based linker:

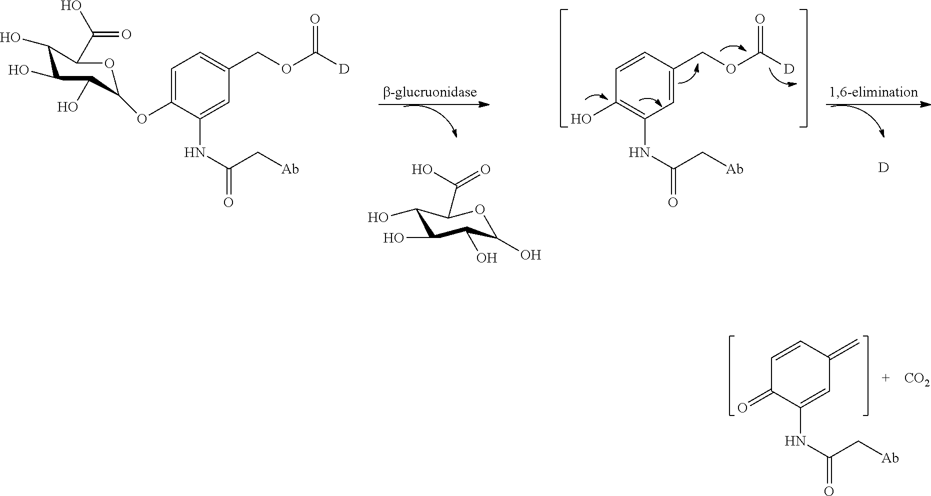

wherein Ab indicates a binding protein of this disclosure.

A variety of cleavable β-glucuronic acid-based linkers useful for linking drugs such as auristatins, camptothecin analogues, doxorubicin analogues, CBI minor-groove binders, and psymberin to antibodies have been described. These β-glucuronic acid-based linkers may be used in the conjugates. In some embodiments, an enzymatically cleavable linker is a β-galactoside-based linker. β-Galactoside is present abundantly within lysosomes, while the enzyme activity outside cells is low.

Additionally, a compound of the present disclosure, or pharmaceutically acceptable isomer, tautomer, racemate, hydrate, solvate, isotope, or salt thereof, containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a compound of any one of compounds of Categories A to K, or pharmaceutically acceptable isomer, tautomer, racemate, hydrate, solvate, isotope, or salt thereof, wherein such ester groups can hydrolyze under physiological conditions to release a compound of any one of compounds of Categories A to K, or pharmaceutically acceptable isomer, tautomer, racemate, hydrate, solvate, isotope, or salt thereof. Hydrolytically degradable linkages can include carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including at the end of a polymer, and a 5′ hydroxyl group of an oligonucleotide.

A linker can contain an enzymatically cleavable peptide, for example, a linker comprising structural formula (LI-CIIIa), (LI-CIIIb), (LI-CIIIc), or (LI-CIIId):

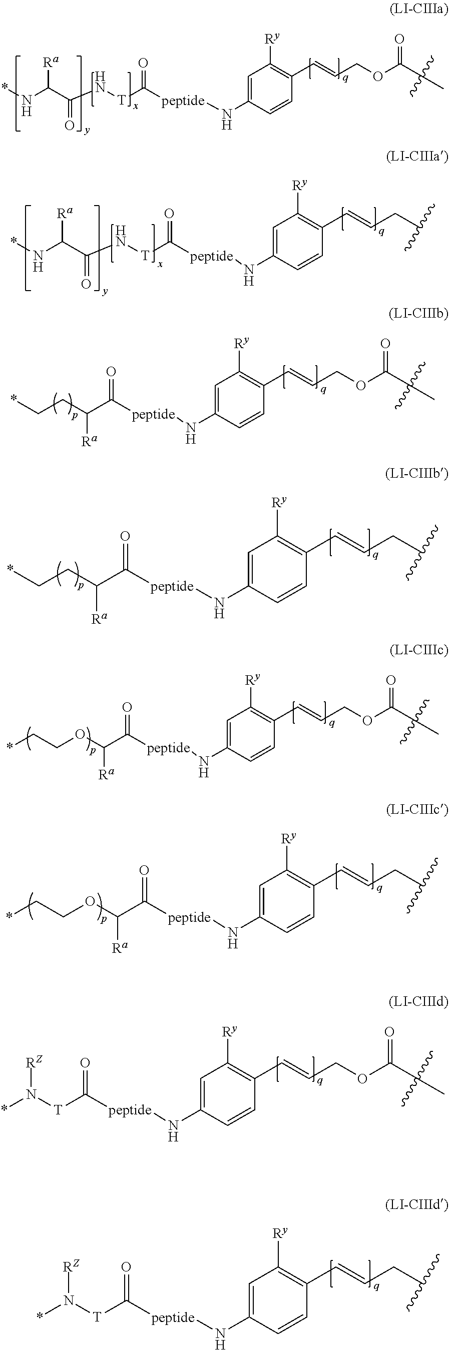

or a salt thereof, wherein: “peptide” represents a peptide (illustrated in N→C orientation, wherein peptide includes the amino and carboxy “termini”) that is cleavable by a lysosomal enzyme; T represents a polymer comprising one or more ethylene glycol units or an alkylene chain, or combinations thereof, R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is —SO₃H, —CO₂H, PEG 4-32, or a sugar moiety; G² is —SO₃H, —CO₂H, or a PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound of the present disclosure, or pharmaceutically acceptable isomer, tautomer, racemate, hydrate, solvate, isotope, or salt thereof; and * represents the point of attachment to the remainder of the linker.

In some embodiments, a peptide can be selected to contain natural amino acids, unnatural amino acids, or any combination thereof. In some embodiments, a peptide can be a tripeptide or a dipeptide. In particular embodiments, a dipeptide comprises L-amino acids, such as Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (LI-Cilia) are illustrated below (as illustrated, the linkers include a reactive group suitable for covalently linking the linker to a binding protein of this disclosure):

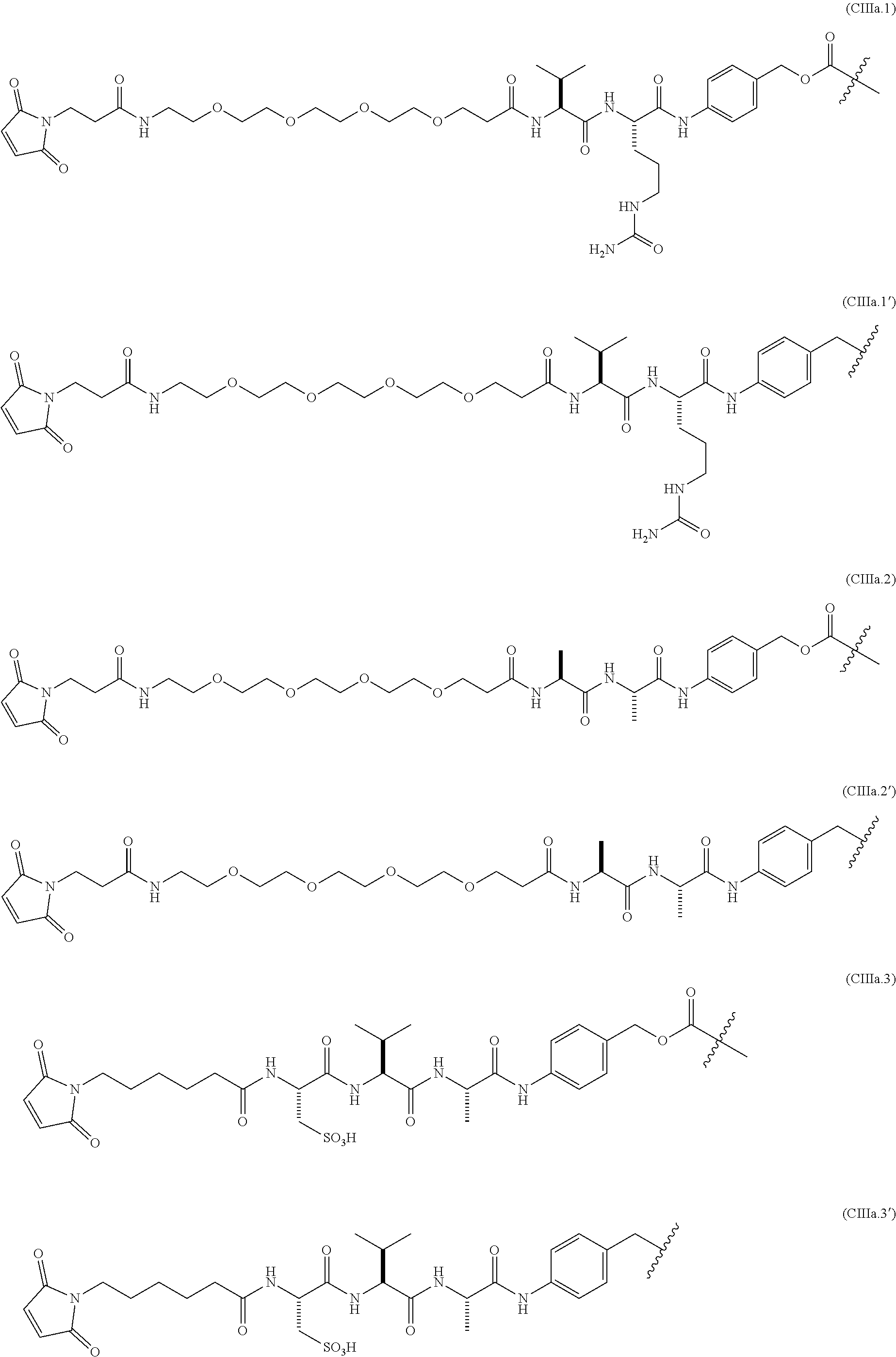

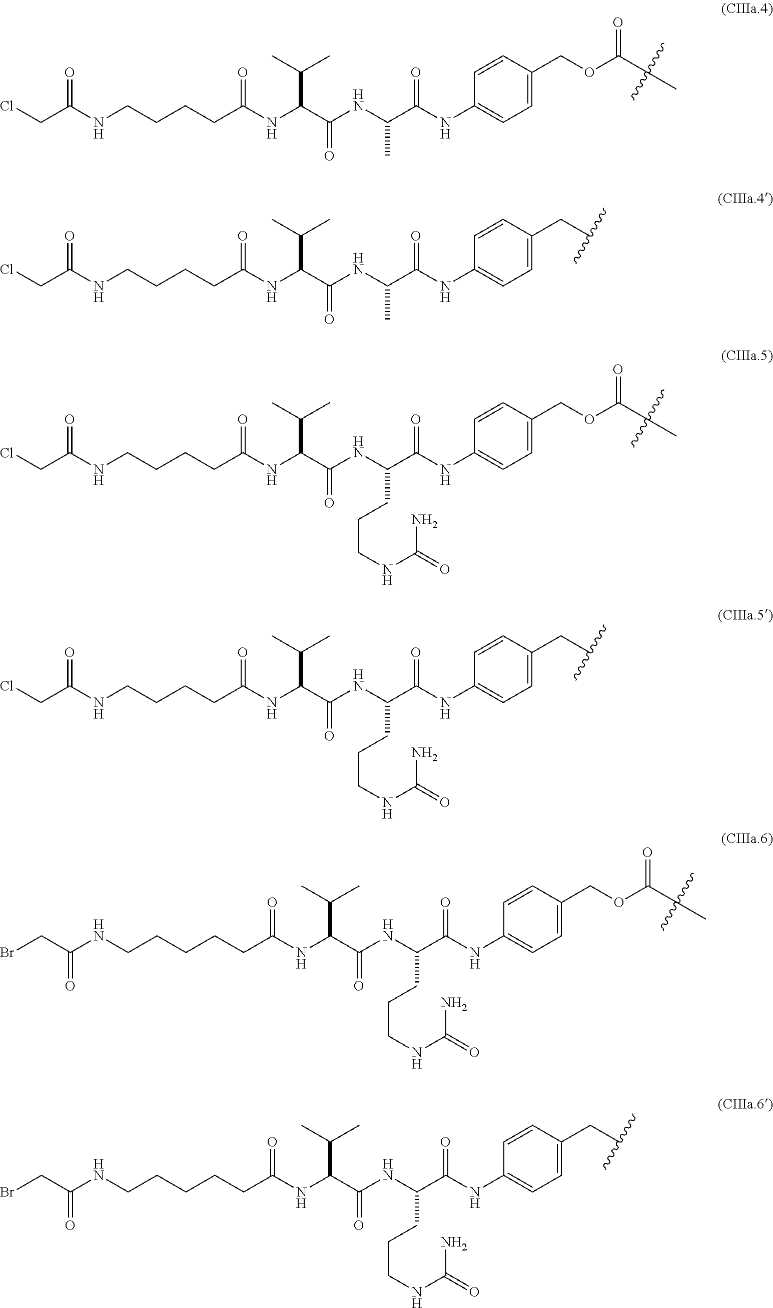 [SMILES: *C(=O)OCc1ccc(NC(=O)[C@H](C)NC(=O)[C@@H](NC(=O)CCCCNC(=O)CCl)C(C)C)cc1 ; *Cc1ccc(NC(=O)[C@H](C)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCNC(=O)CCl)C(C)C)cc1 ; *Cc1ccc(NC(=O)[C@H](CCCNC(N)=O)NC(=O)[C@@H](NC(=O)CCCCNC(=O)CCl)C(C)C)cc1 ; *C(=O)OCc1ccc(NC(=O)[C@H](CCCNC(N)=O)NC(=O)[C@@H](NC(=O)CCCCCNC(=O)CBr)C(C)C)cc1 ; *Cc1ccc(NC(=O)[C@H](CCCNC(N)=O)NC(=O)[C@@H](NC(=O)CCCCCNC(=O)CBr)C(C)C)cc1]

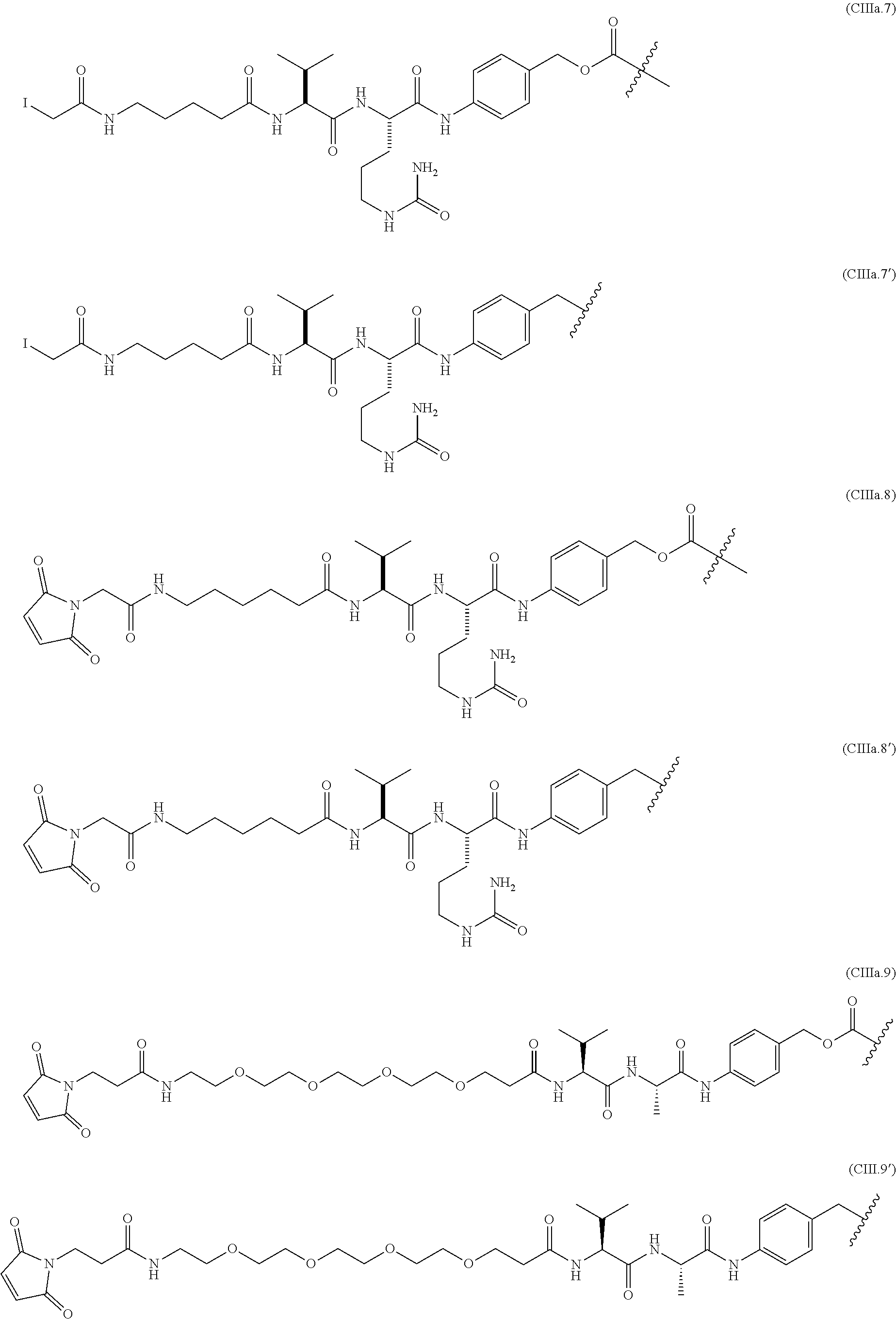

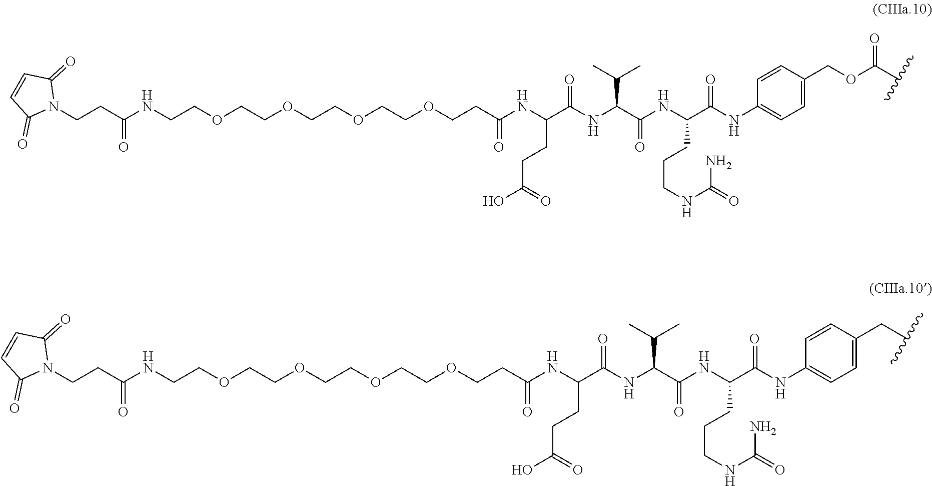 [SMILES: *C(=O)OCc1ccc(NC(=O)[C@H](CCCNC(N)=O)NC(=O)[C@@H](NC(=O)C(CCC(=O)O)NC(=O)CCOCCOCCOCCOCCNC(=O)CCN2C(=O)C=CC2=O)C(C)C)cc1 ; *Cc1ccc(NC(=O)[C@H](CCCNC(N)=O)NC(=O)[C@@H](NC(=O)C(CCC(=O)O)NC(=O)CCOCCOCCOCCOCCNC(=O)CCN2C(=O)C=CC2=O)C(C)C)cc1]

wherein [symbol] indicates an attachment site of a linker (L) to a compound of the present disclosure, or pharmaceutically acceptable isomer, tautomer, racemate, hydrate, solvate, isotope, or salt thereof.

Exemplary embodiments of linkers according to structural formula (CIIIb), (CIIIc), or (CIIId) that can be included in the conjugates can include the linkers illustrated below (as illustrated, the linkers include a reactive group suitable for covalently linking the linker to a polypeptide comprising a binding domain, such as a fusion protein, or an antibody or an antigen-binding fragment thereof):

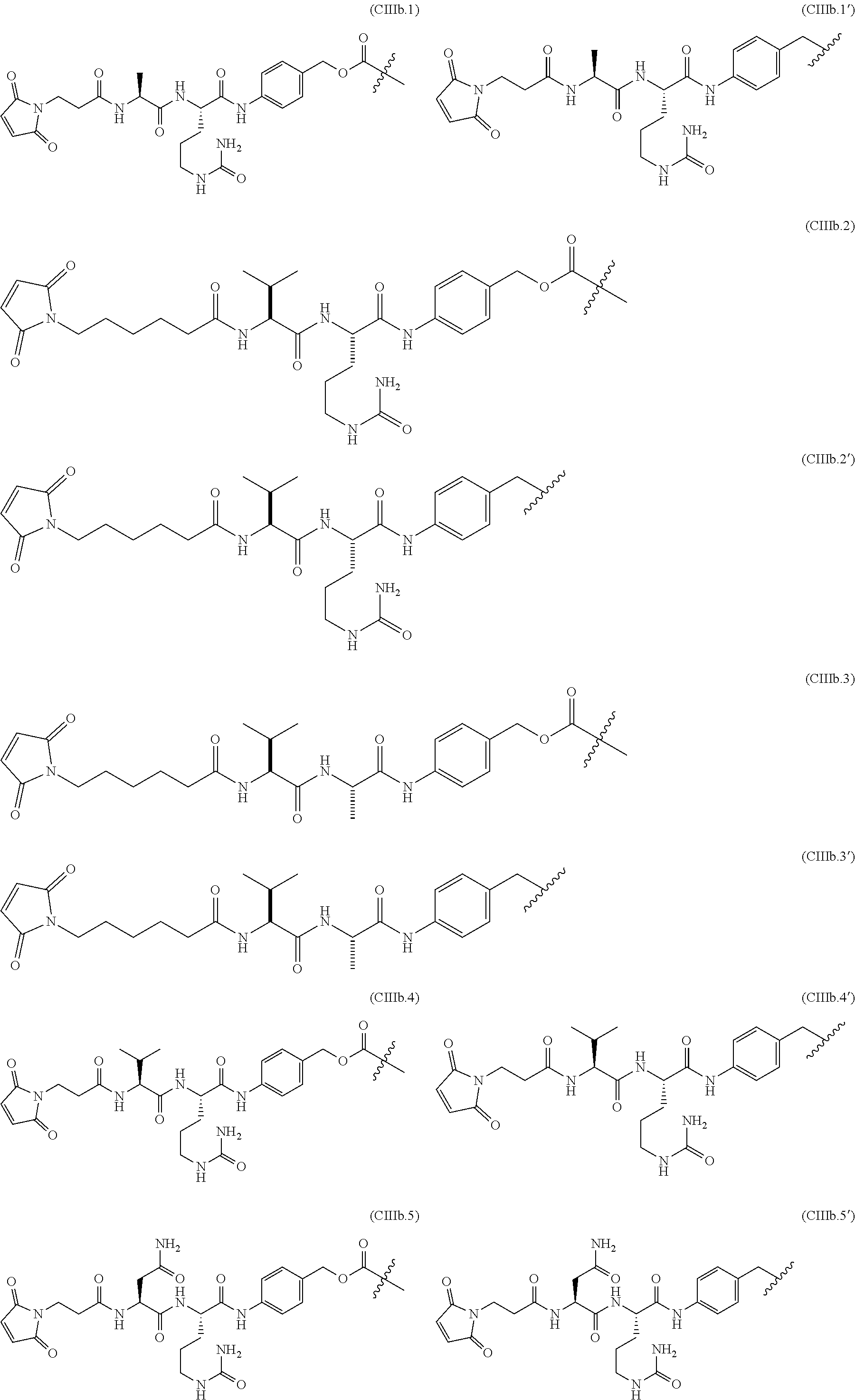

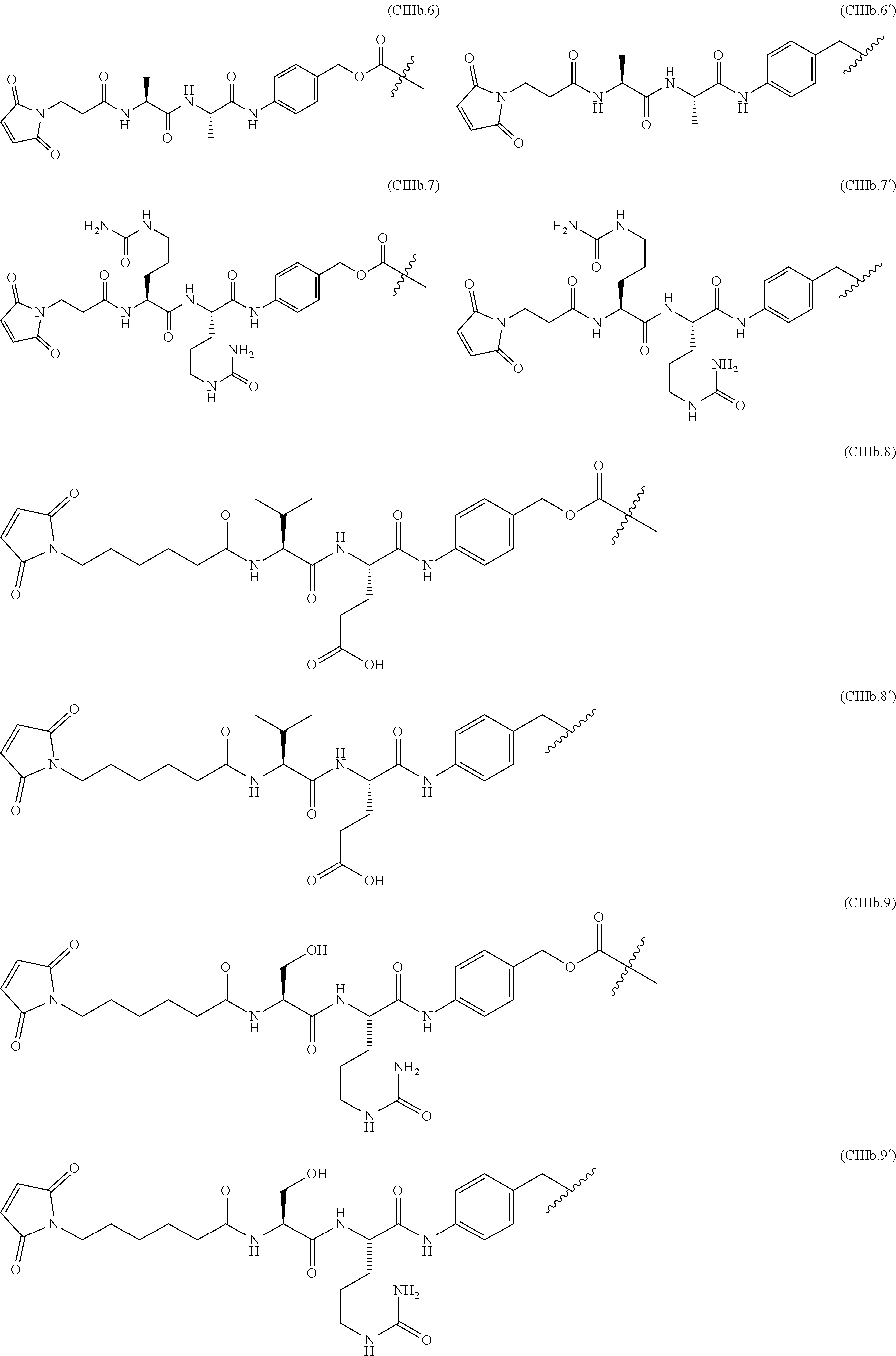

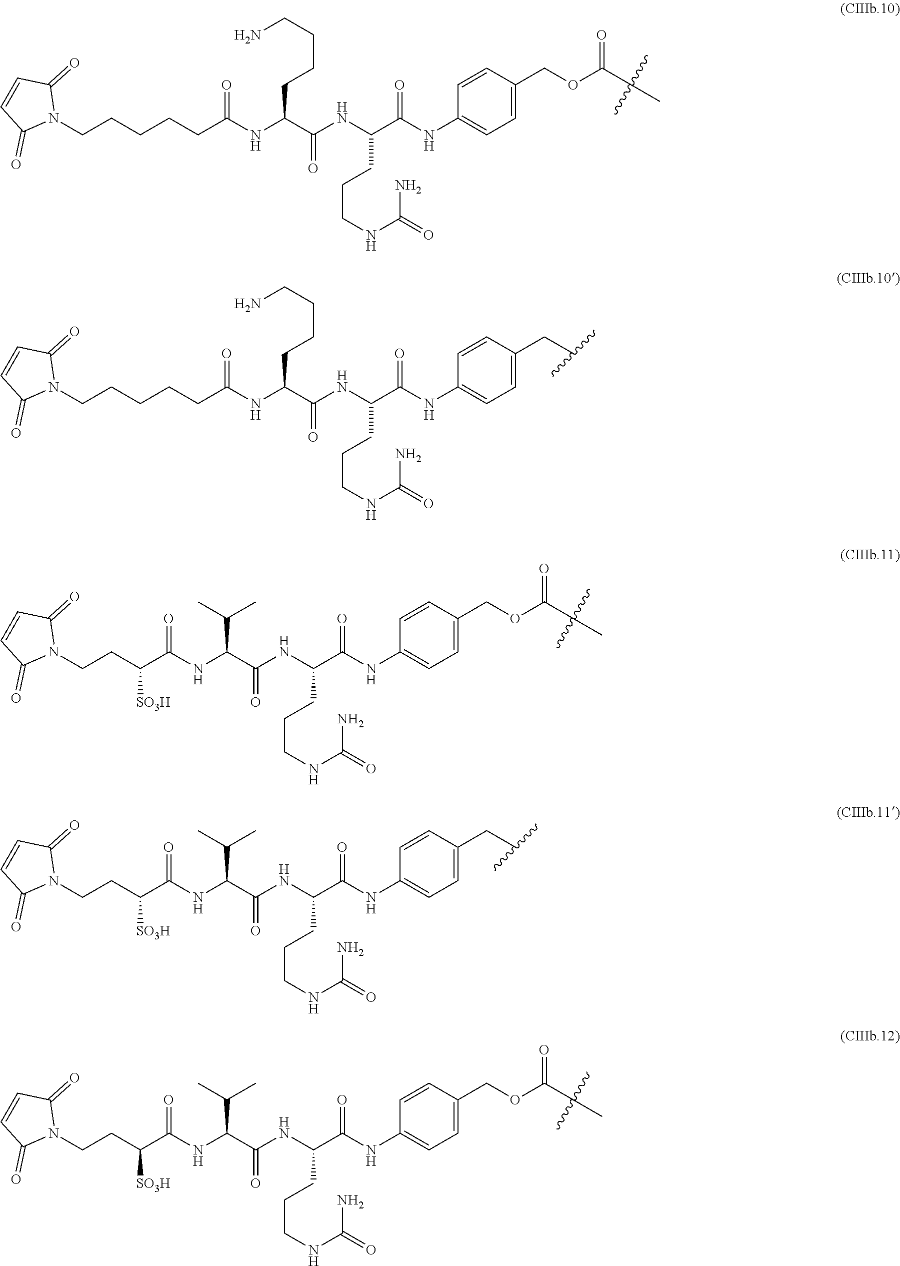 [SMILES: *C(=O)OCc1ccc(NC(=O)[C@H](CCCNC(N)=O)NC(=O)[C@H](CCCCN)NC(=O)CCCCCN2C(=O)C=CC2=O)cc1 ; *Cc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *Cc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

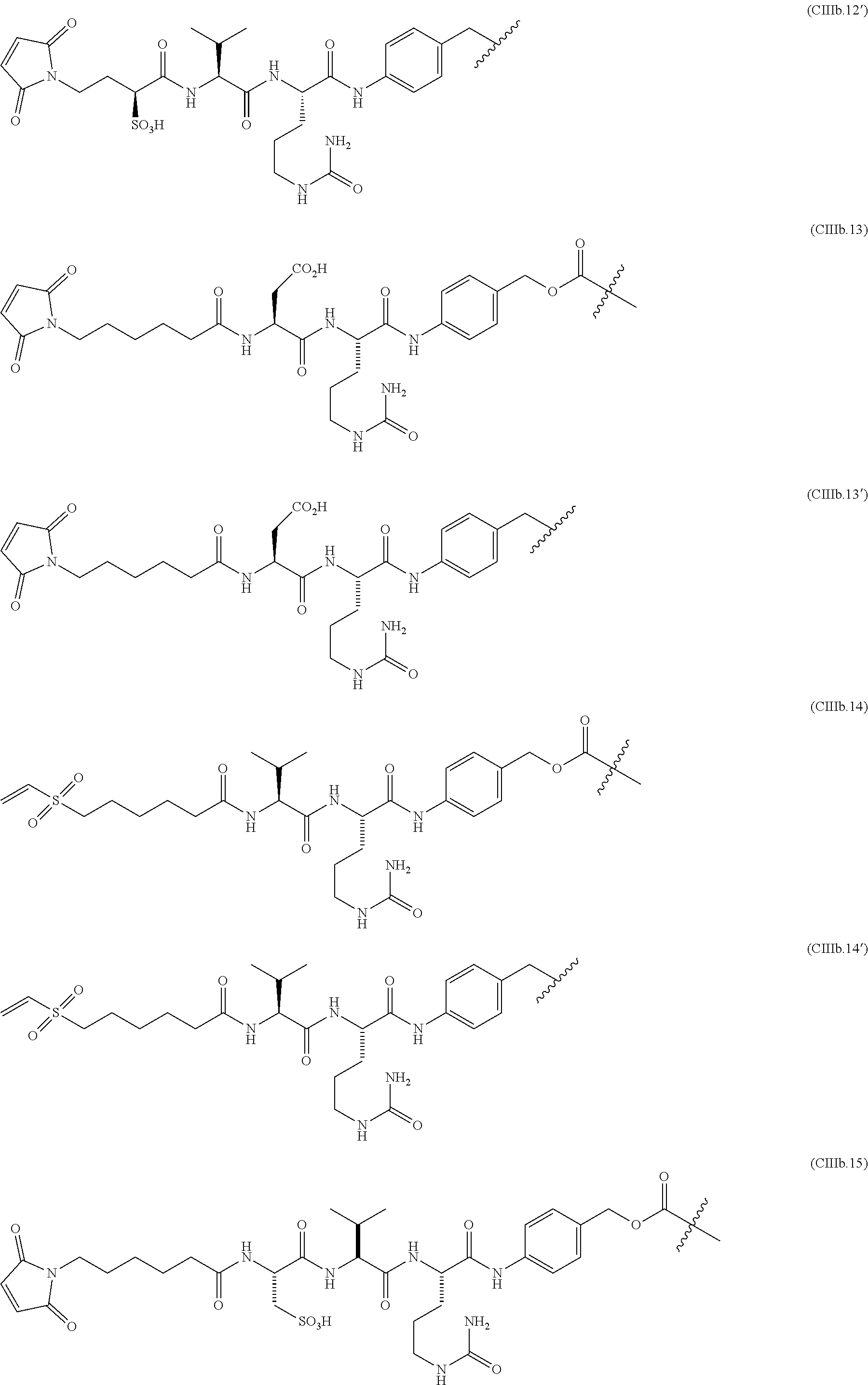

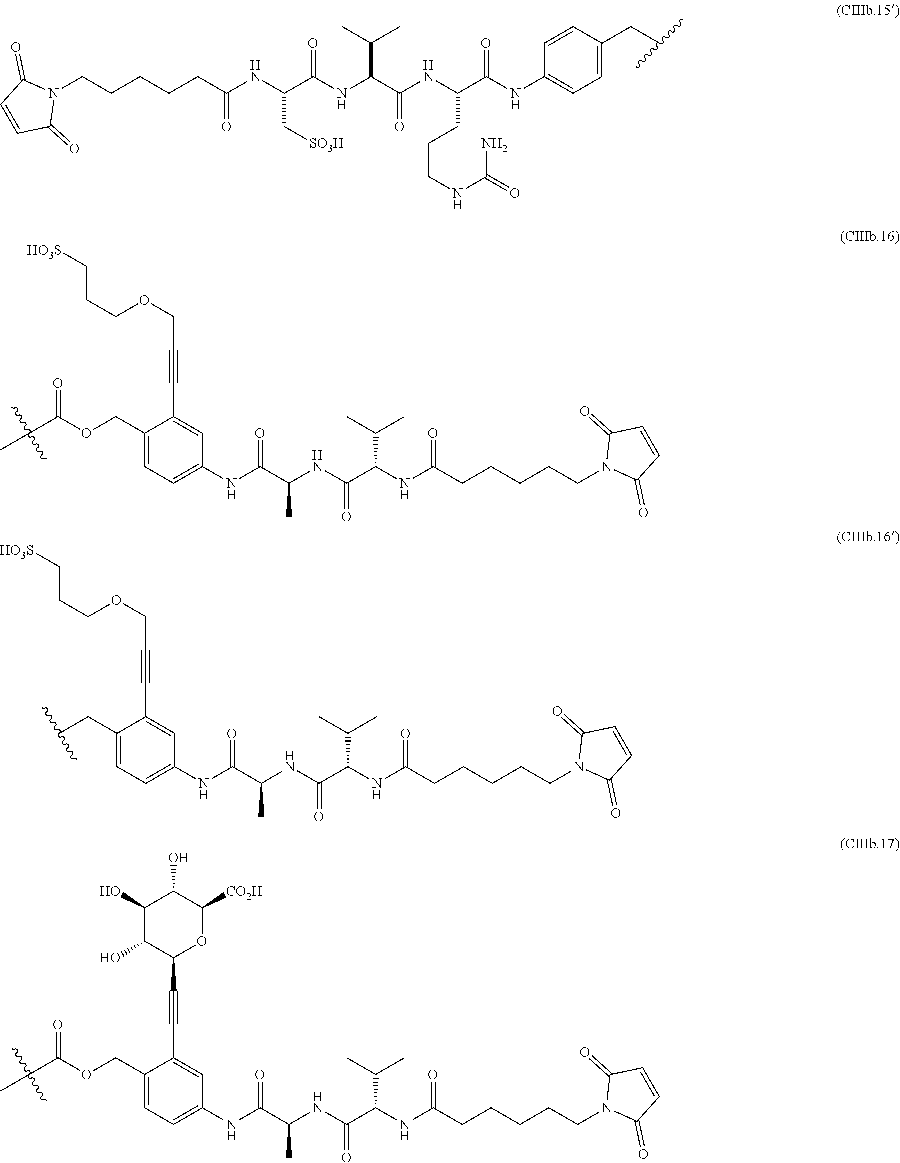 [SMILES: *Cc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *Cc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O]

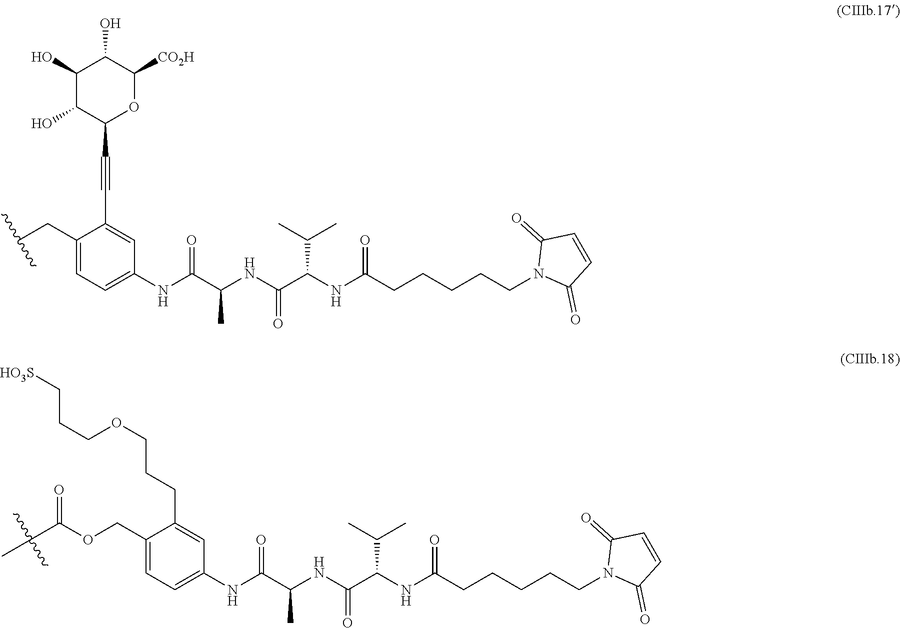 [SMILES: *Cc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O]

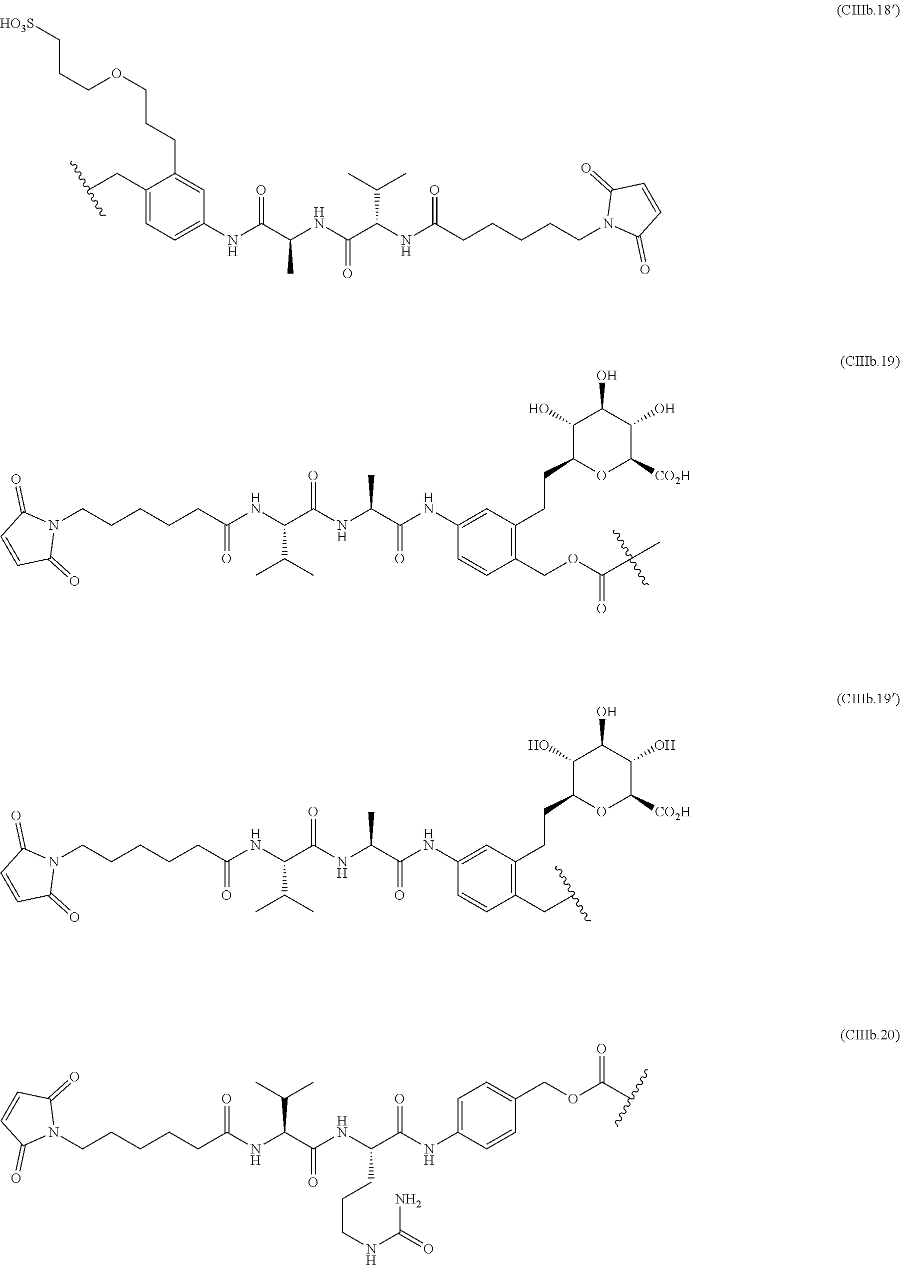 [SMILES: *Cc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *Cc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)CCCCCN2C(=O)C=CC2=O)C(C)C)cc1]

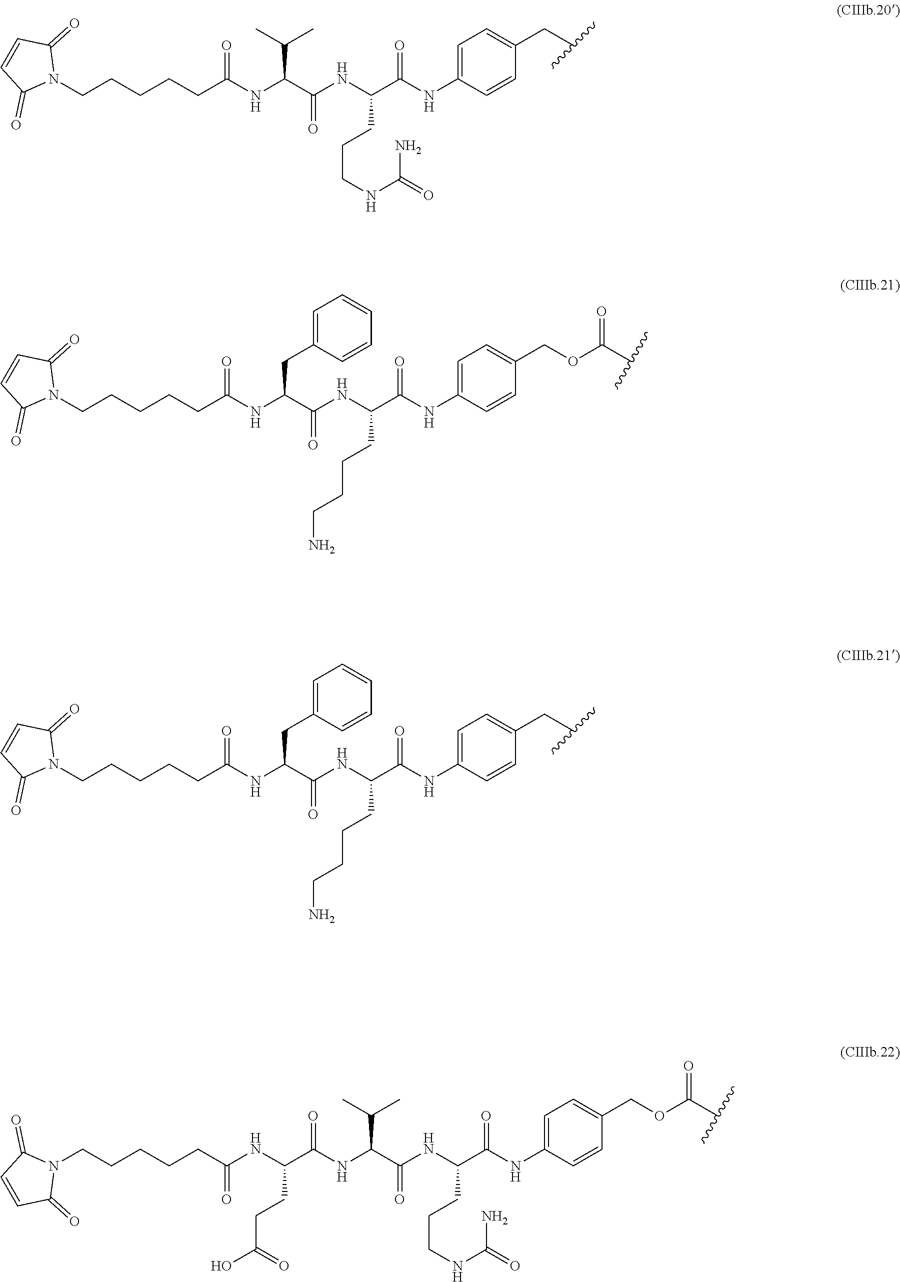 [SMILES: *Cc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](CCCCN)NC(=O)[C@H](Cc2ccccc2)NC(=O)CCCCCN2C(=O)C=CC2=O)cc1 ; *Cc1ccc(NC(=O)[C@H](CCCCN)NC(=O)[C@H](Cc2ccccc2)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@H](CCC(=O)O)NC(=O)CCCCCN2C(=O)C=CC2=O)C(C)C)cc1]

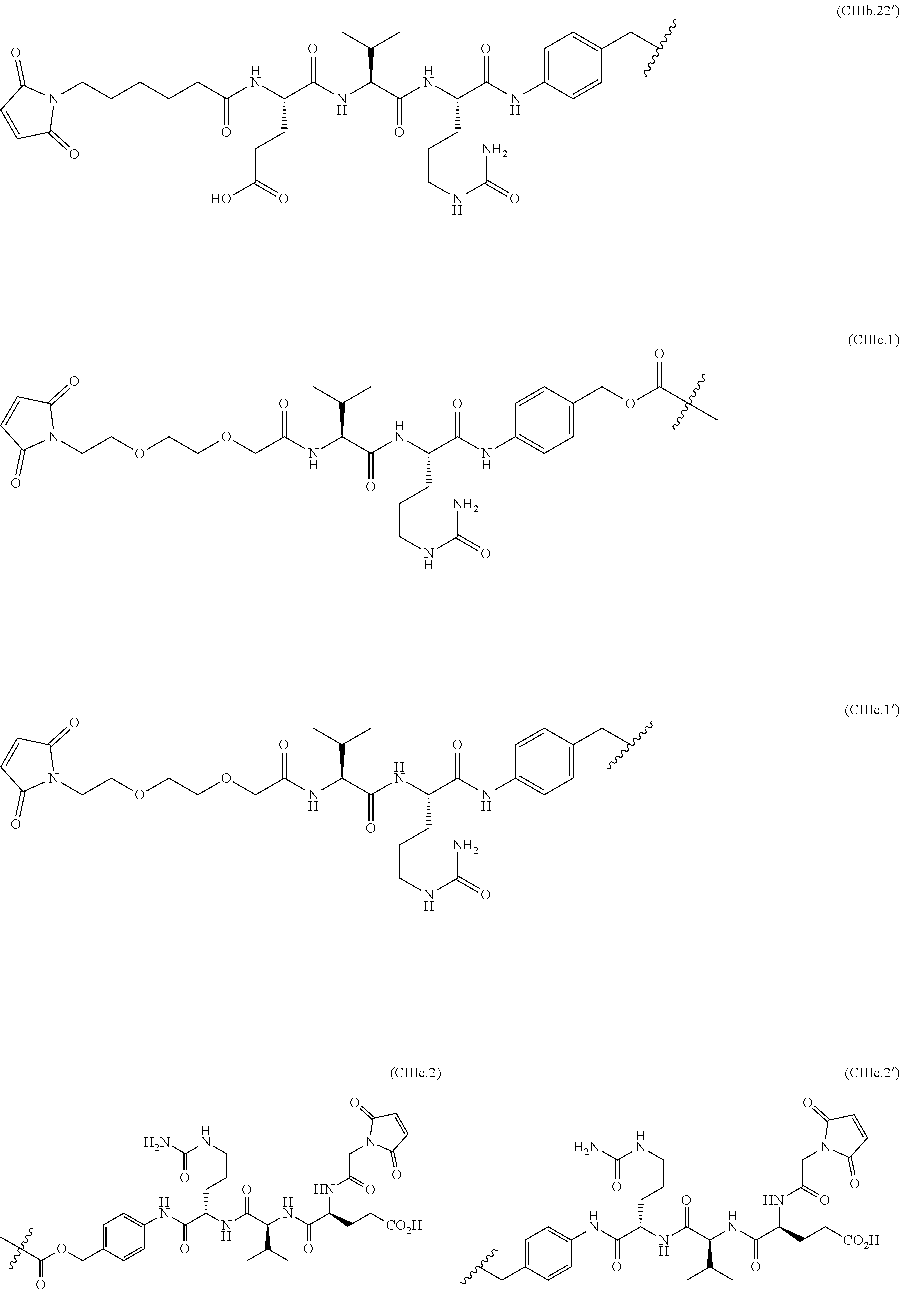 [SMILES: *Cc1ccc(NC(=O)[C@H](CCCNC(N)=O)NC(=O)[C@@H](NC(=O)[C@H](CCC(=O)O)NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)COCCOCCN2C(=O)C=CC2=O)C(C)C)cc1 ; *Cc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *Cc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1]

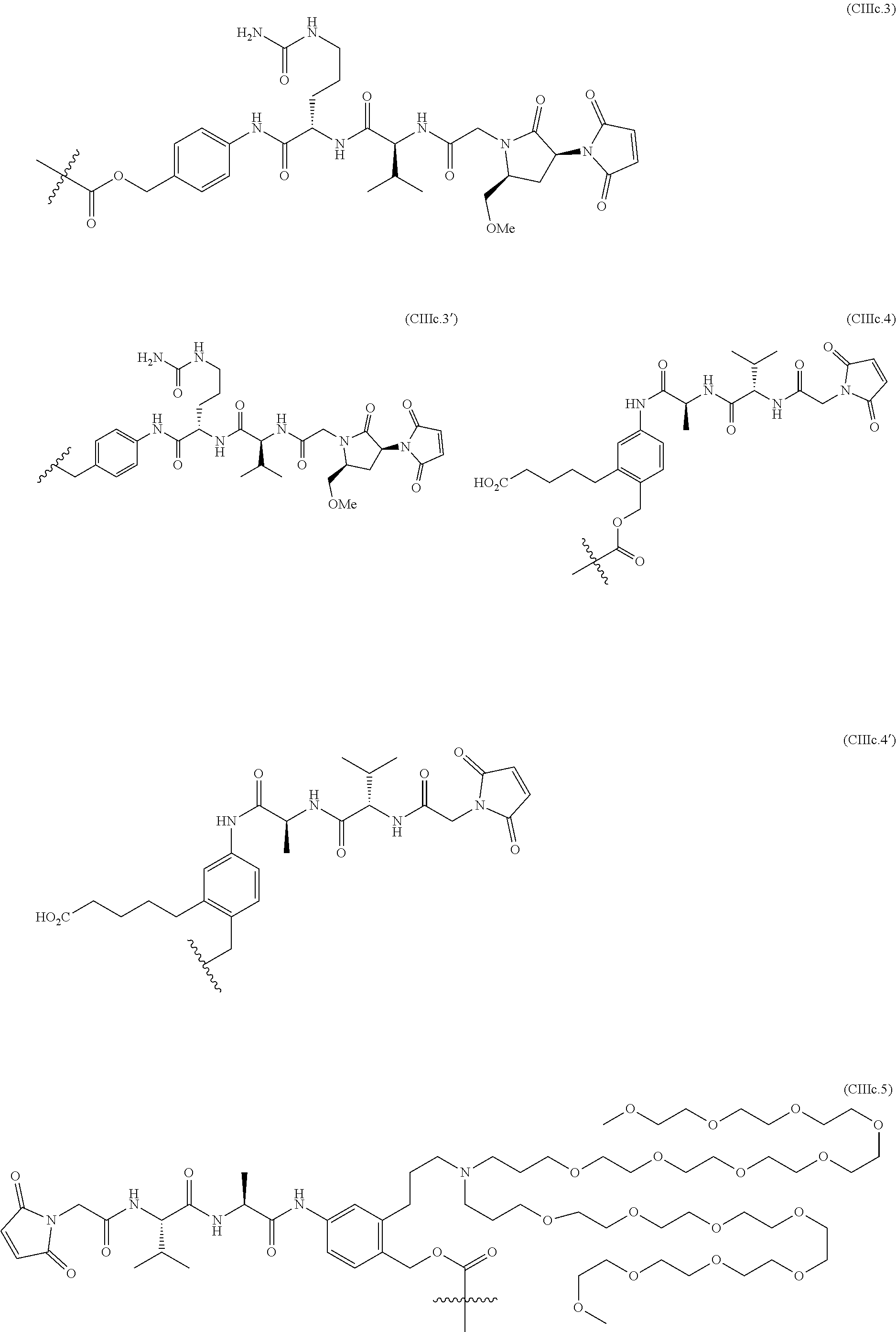

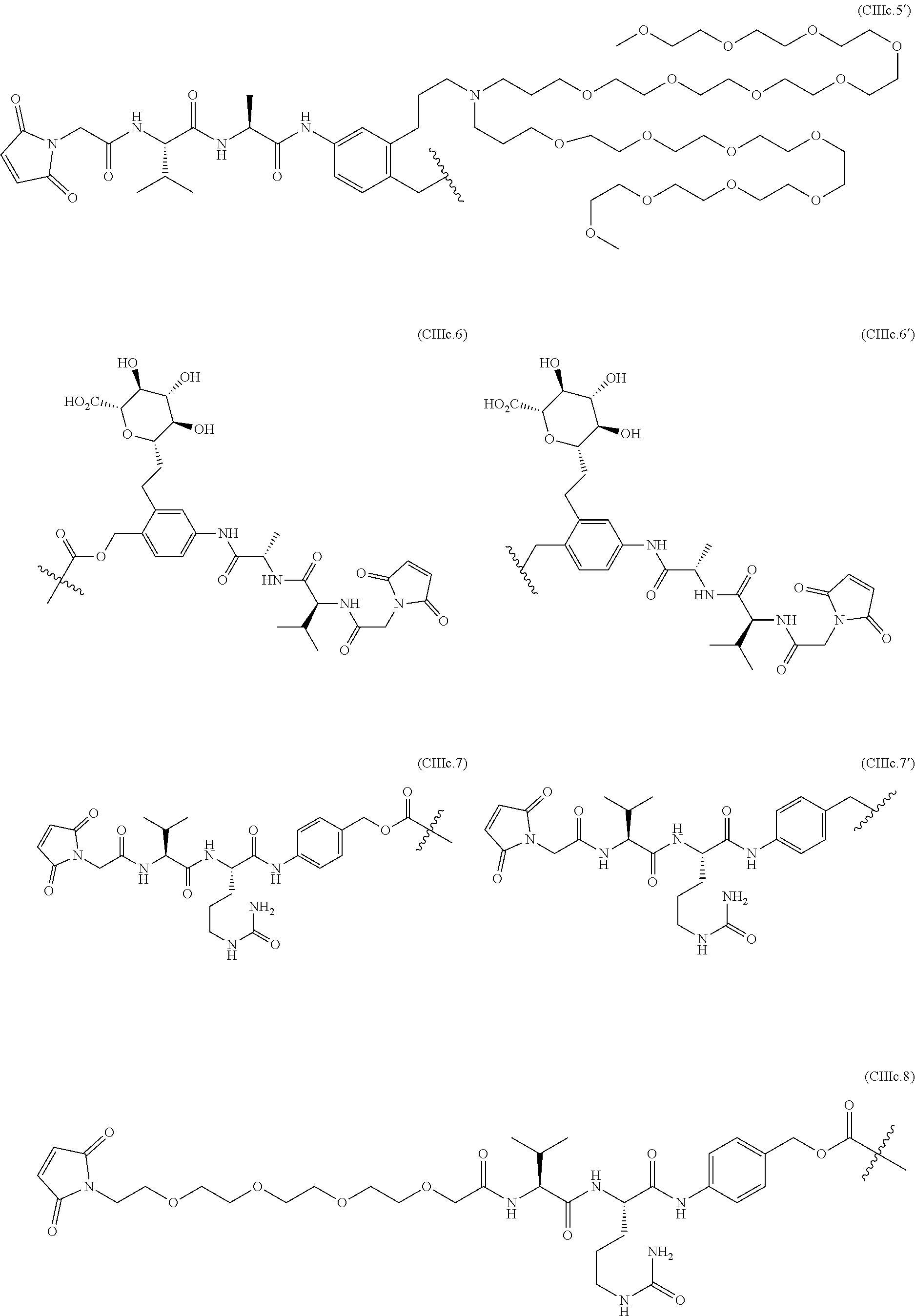

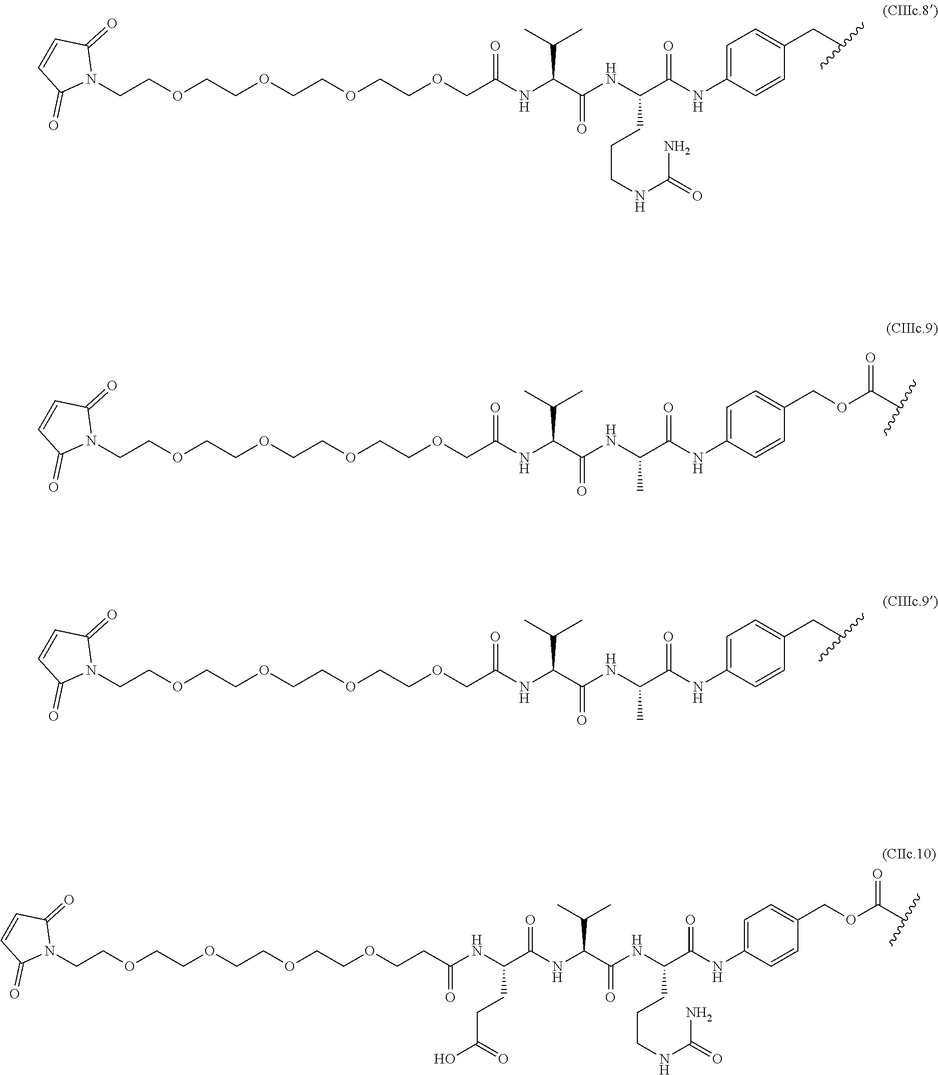 [SMILES: *Cc1ccc(NC(=O)[C@H](CCCNC(N)=O)NC(=O)[C@@H](NC(=O)COCCOCCOCCOCCN2C(=O)C=CC2=O)C(C)C)cc1 ; *C(=O)OCc1ccc(NC(=O)[C@H](C)NC(=O)[C@@H](NC(=O)COCCOCCOCCOCCN2C(=O)C=CC2=O)C(C)C)cc1 ; *Cc1ccc(NC(=O)[C@H](C)NC(=O)[C@@H](NC(=O)COCCOCCOCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CCOCCOCCOCCOCCN2C(=O)C=CC2=O)C(C)C)cc1]

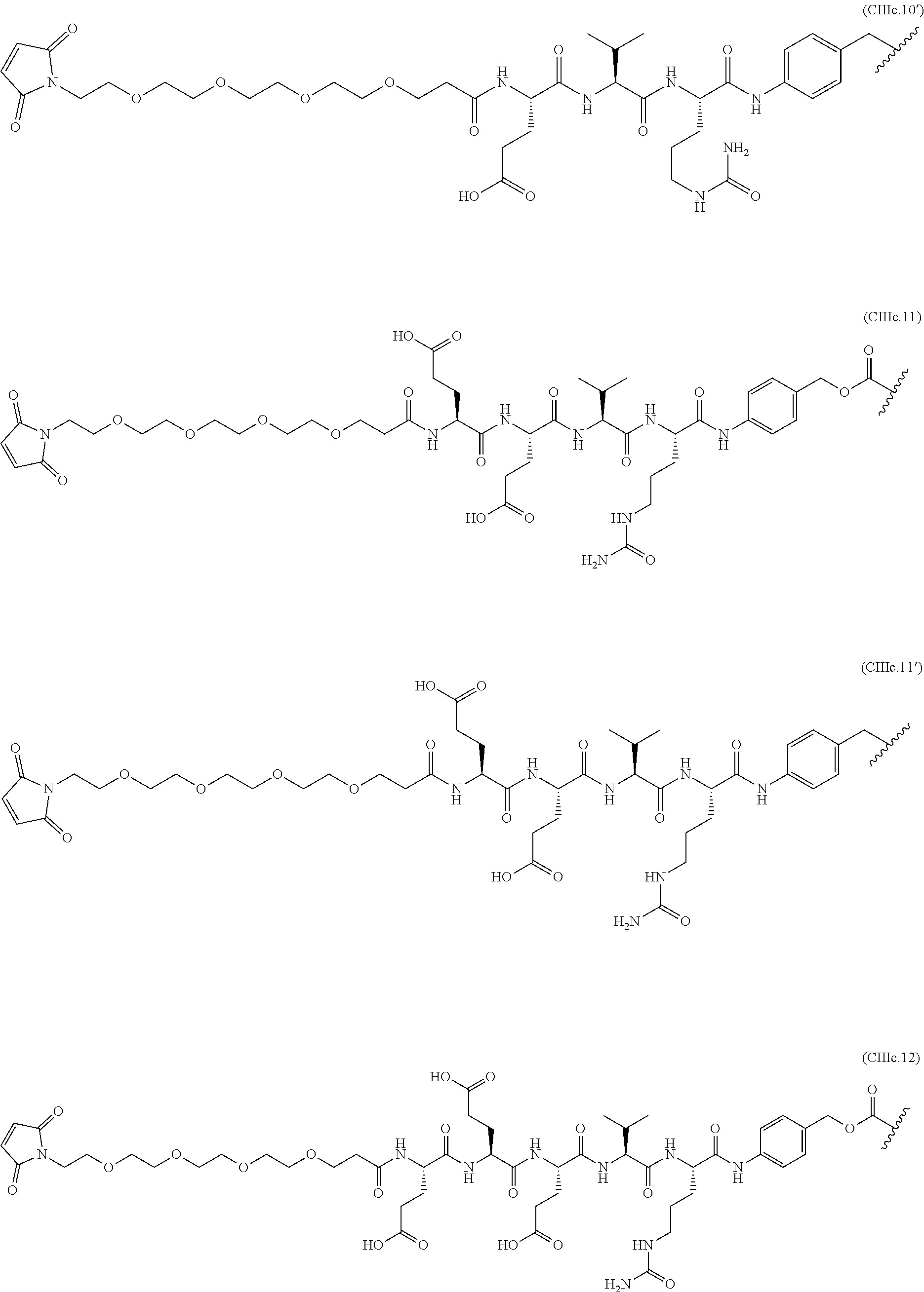

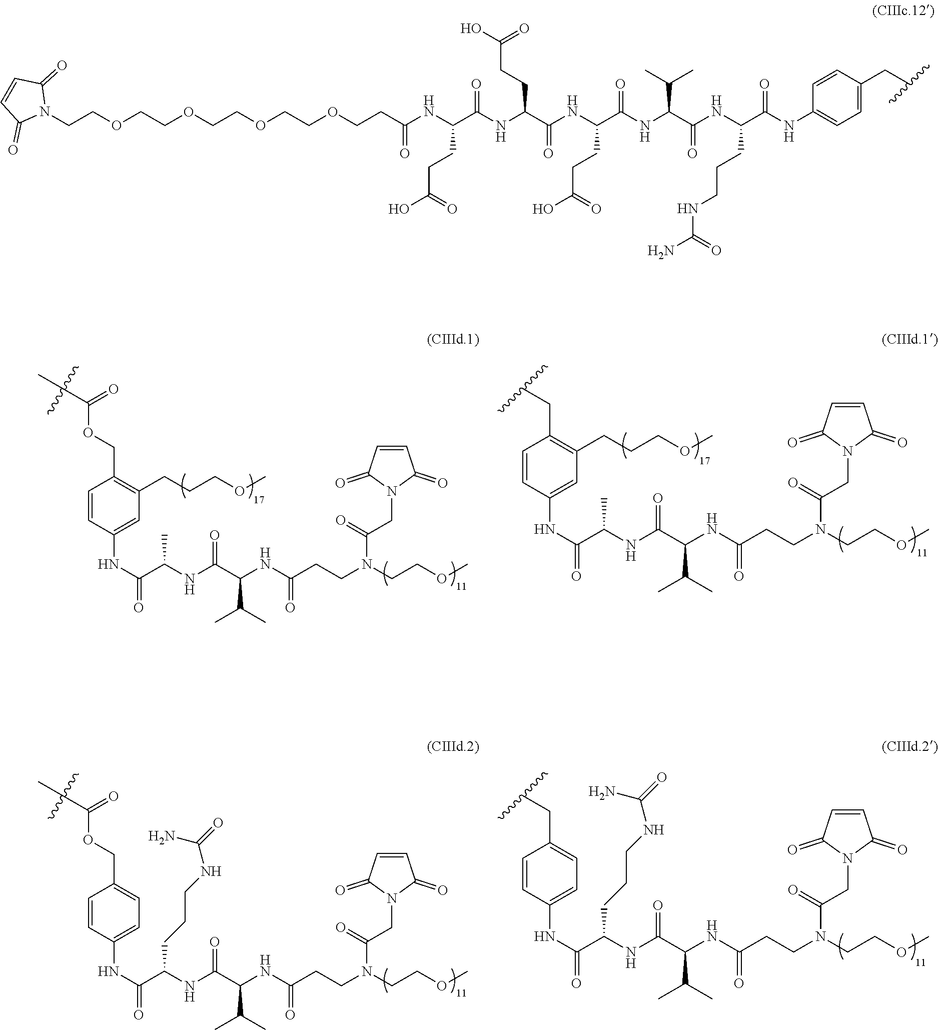

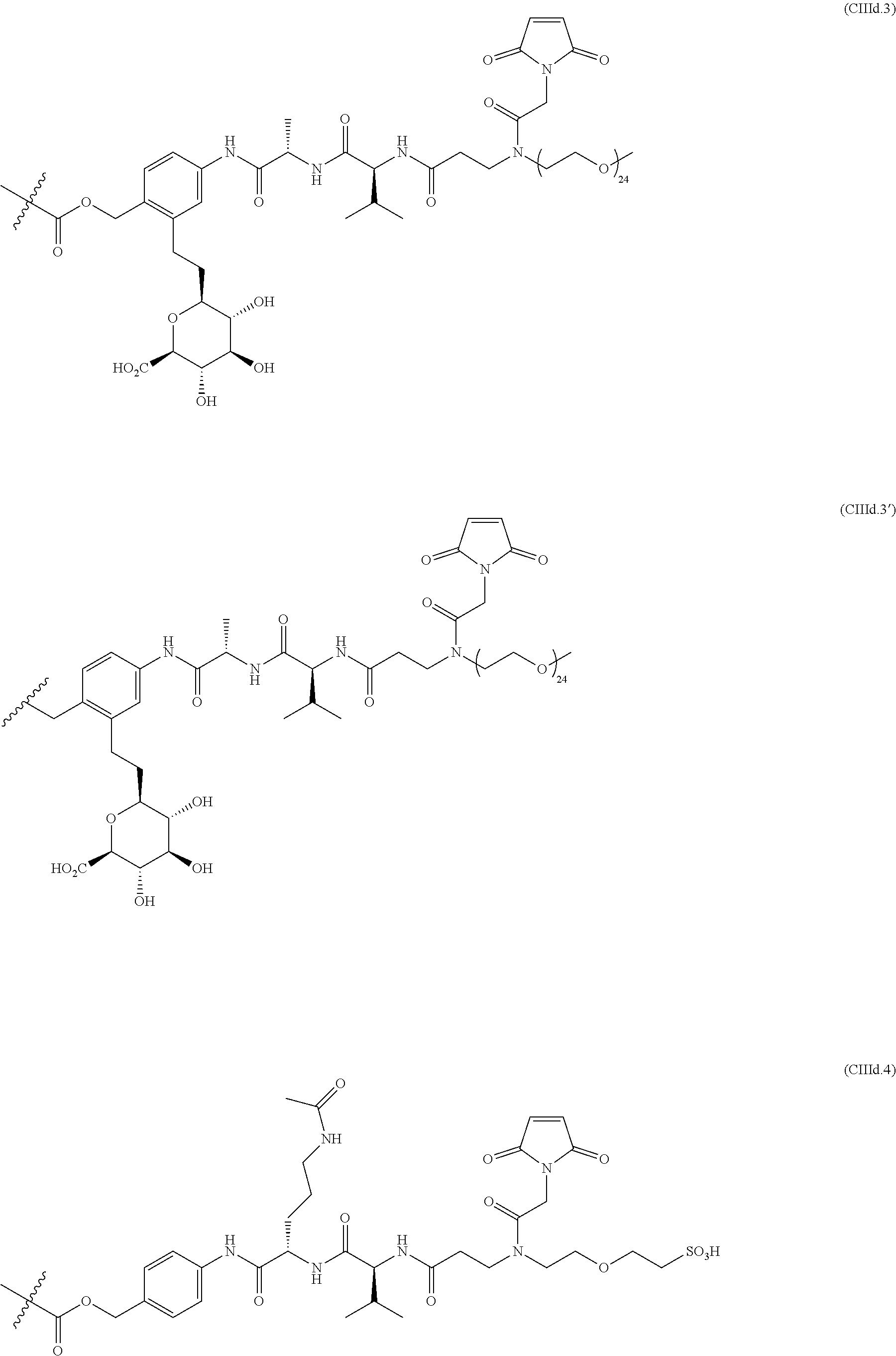

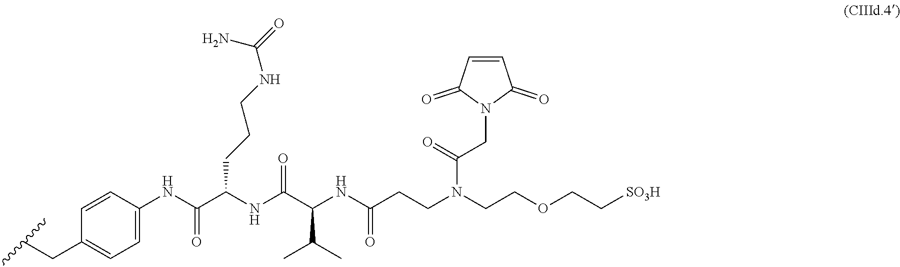 [SMILES: *Cc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

wherein [symbol] indicates an attachment site to a compound of any one of structure (Iq) or (IIq), or pharmaceutically acceptable isomer, tautomer, racemate, hydrate, solvate, isotope, or salt thereof.

The linker can contain an enzymatically cleavable sugar moiety, for example, a linker comprising structural formula (CIVa), (CIVb), (CIVc), (CIVd), or (CIVe):

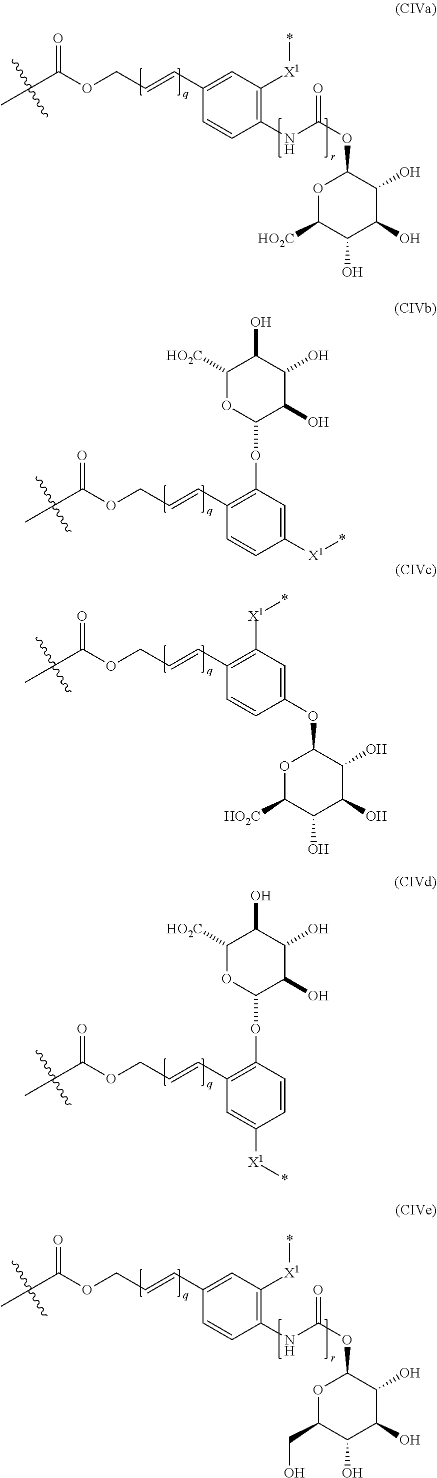

or a salt thereof, wherein: q is 0 or 1; r is 0 or 1; X¹ is CH₂, O or NH; [symbol] represents the point of attachment of the linker (L) to a compound of the present disclosure, or pharmaceutically acceptable isomer, tautomer, racemate, hydrate, solvate, isotope, or salt thereof; and * represents the point of attachment to the remainder of the linker.

Exemplary embodiments of linkers according to structural formula (CIVa) that may be included in the antibody construct conjugates of a compound of the present disclosure, or pharmaceutically acceptable isomer, tautomer, racemate, hydrate, solvate, isotope, or salt thereof, of the disclosure can include the linkers illustrated below (as illustrated, the linkers include a group suitable for covalently linking the linker to a binding protein of this disclosure):

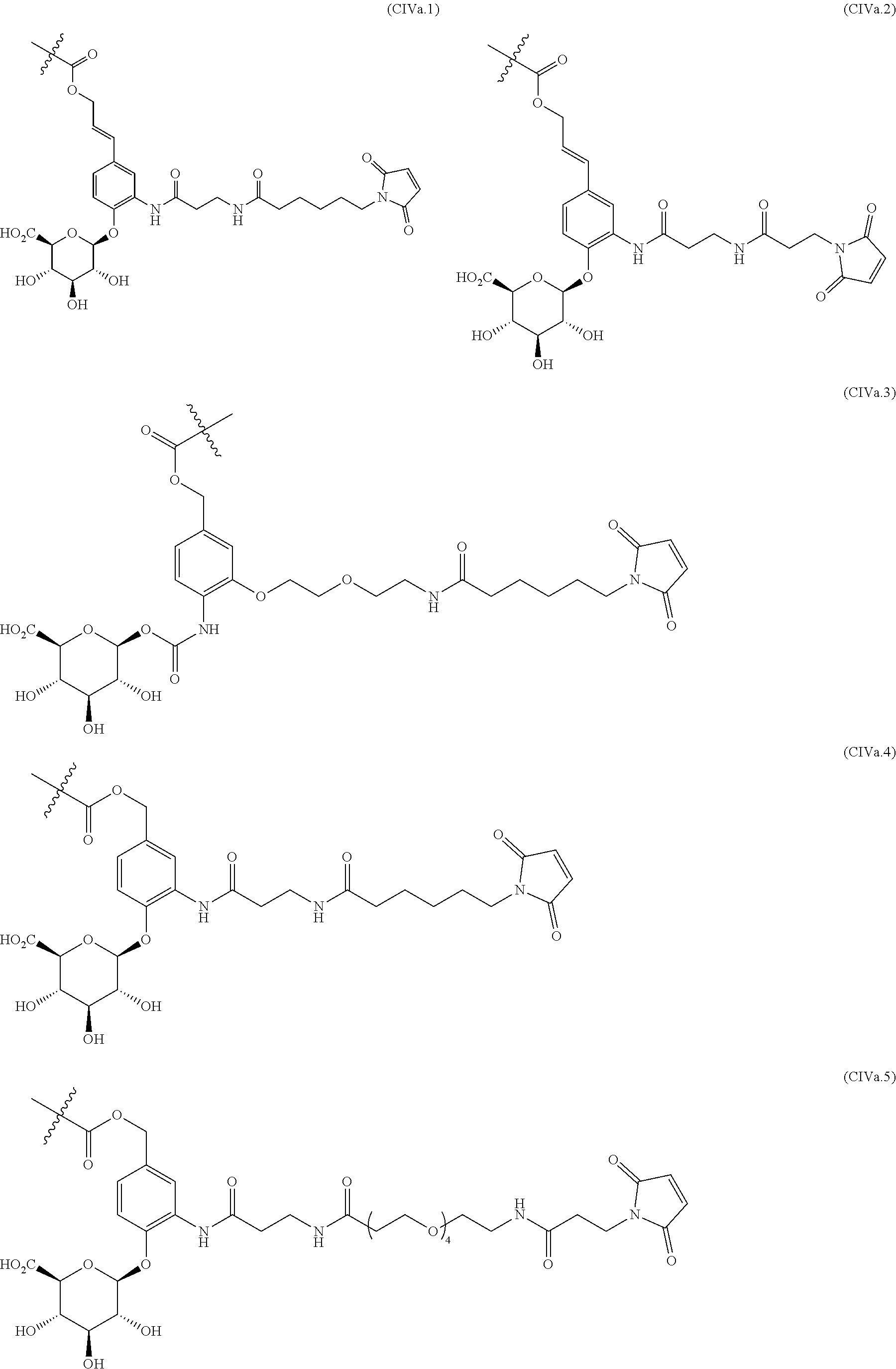

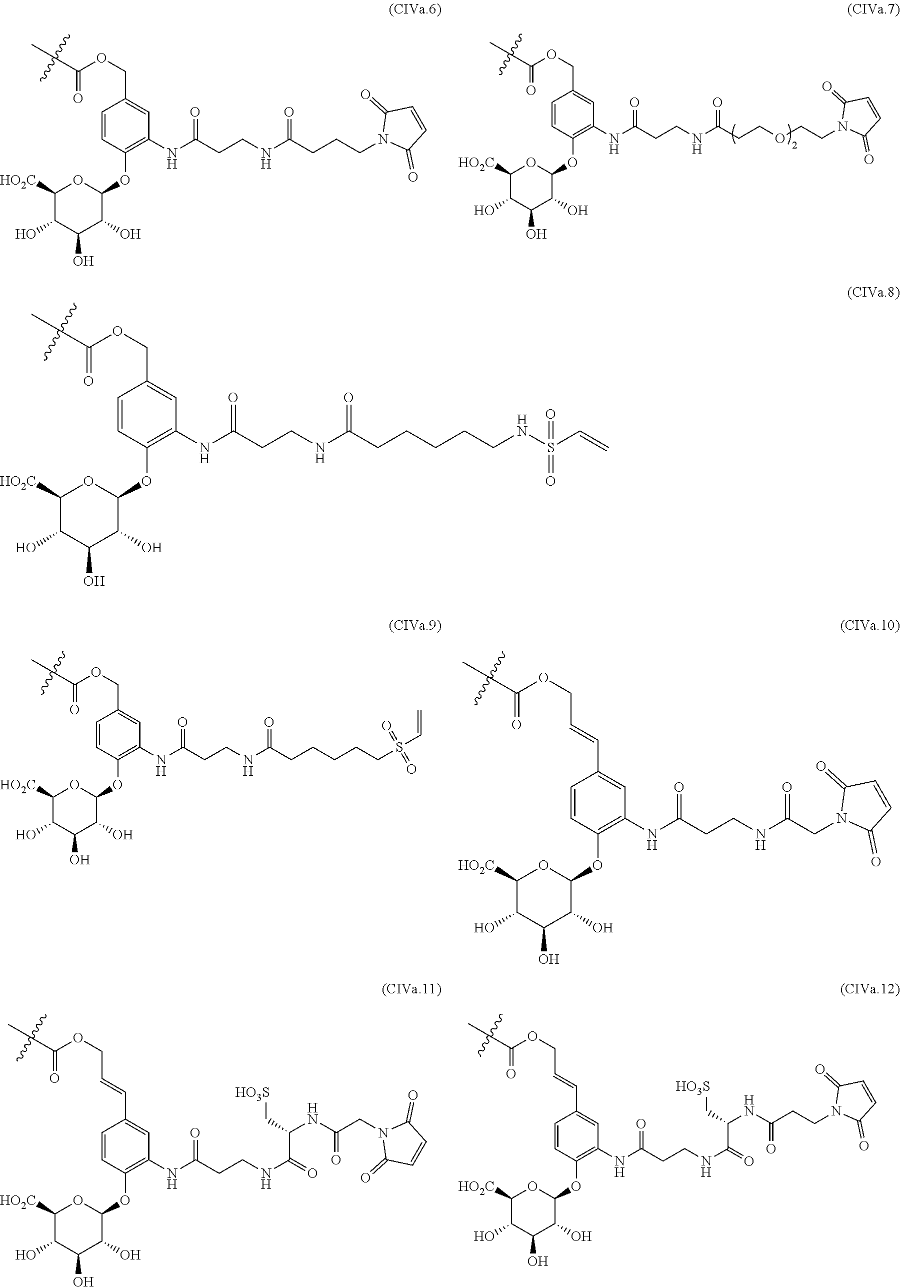

wherein [symbol] represents the point of attachment of the linker (L) to a compound of the present disclosure, or pharmaceutically acceptable isomer, tautomer, racemate, hydrate, solvate, isotope, or salt thereof.

Exemplary embodiments of linkers according to structural formula (CIVb) that may be included in the conjugates include the linkers illustrated below (as illustrated, the linkers include a group suitable for covalently linking the linker to a binding protein of this disclosure):

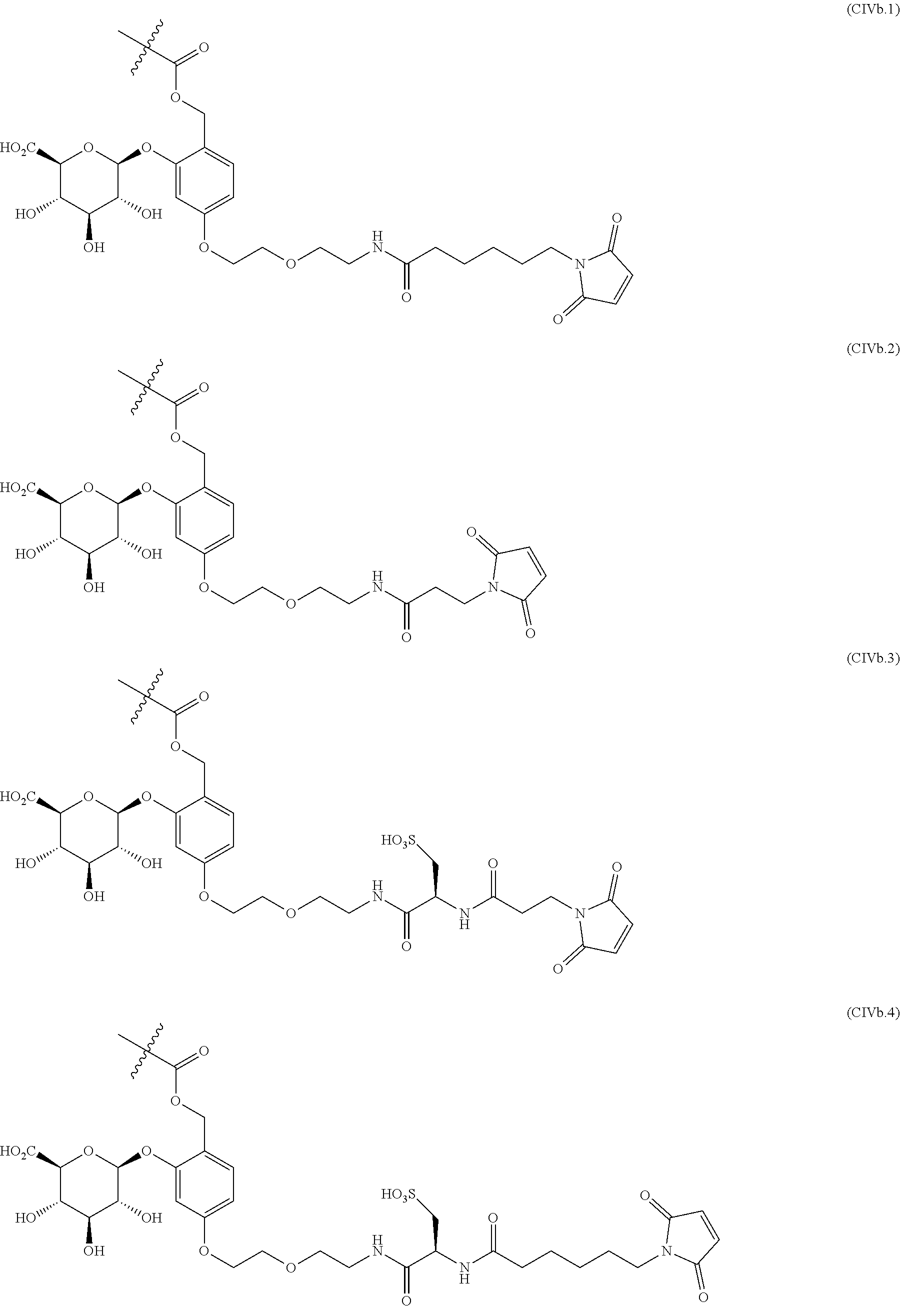

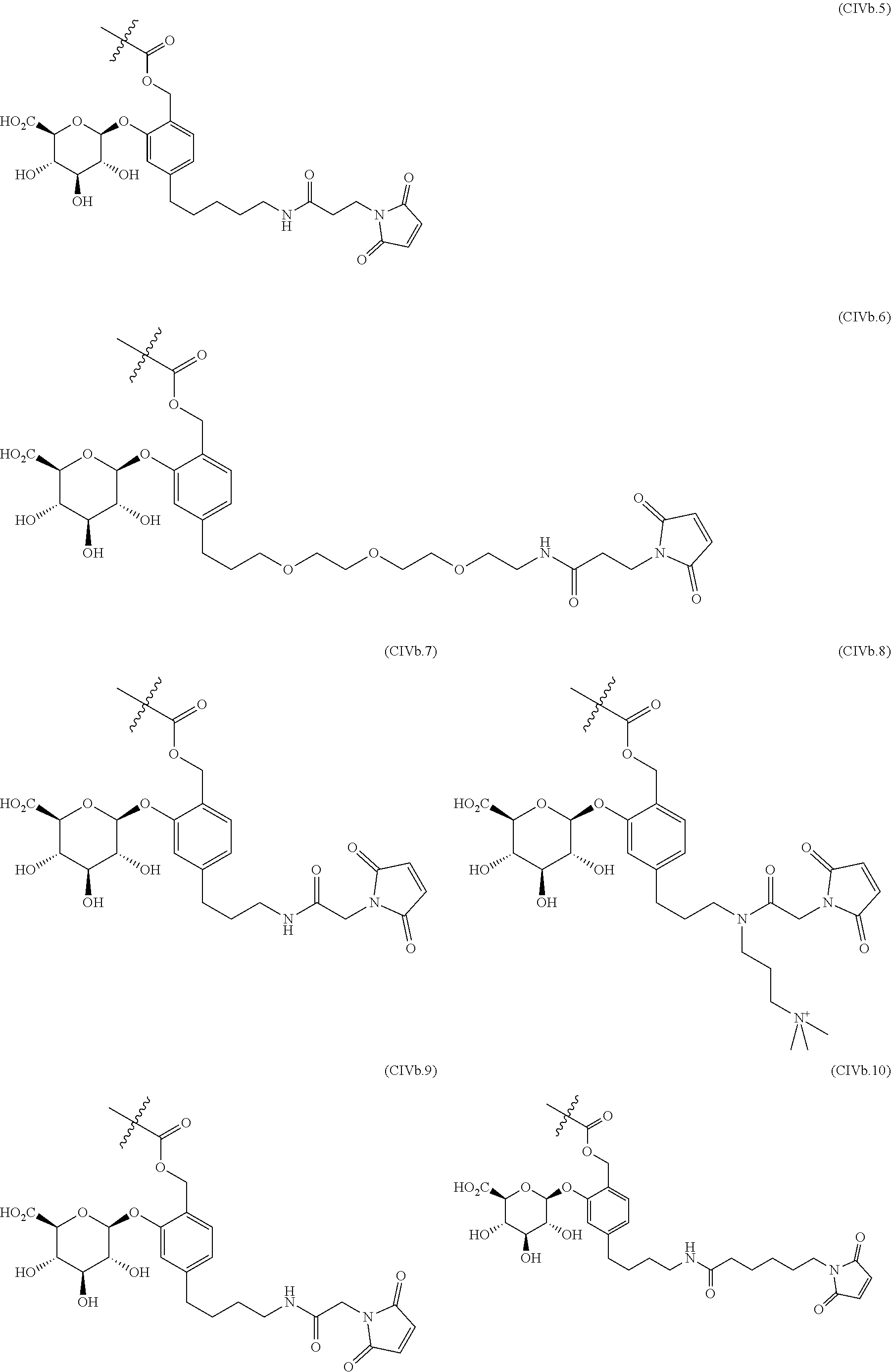

wherein [symbol] represents the point of attachment of the linker (L) to a compound of the present disclosure, or pharmaceutically acceptable isomer, tautomer, racemate, hydrate, solvate, isotope, or salt thereof.

Exemplary embodiments of linkers according to structural formula (CIVc) that may be included in the conjugates include the linkers illustrated below (as illustrated, the linkers include a group suitable for covalently linking the linker to a binding protein of this disclosure):

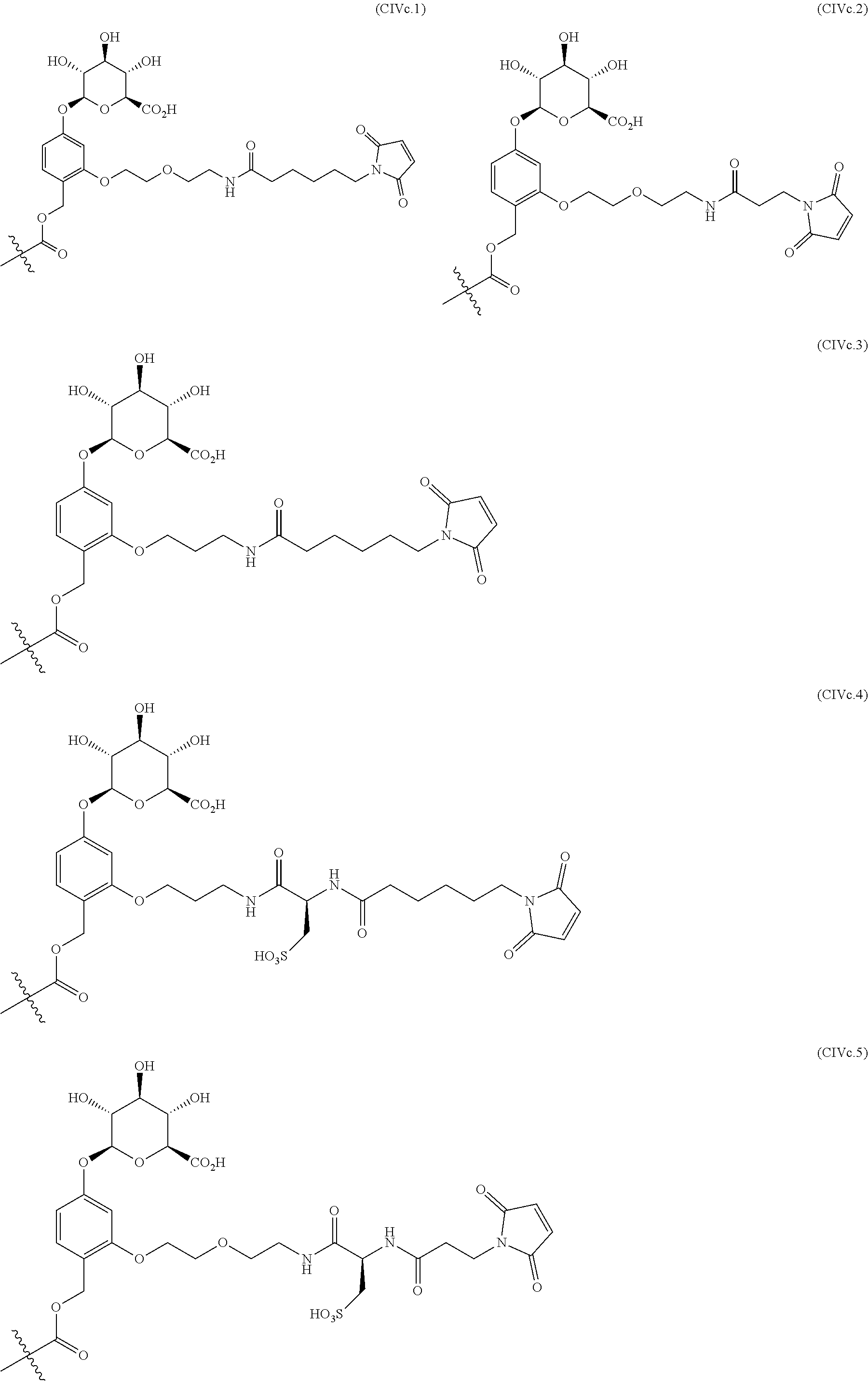

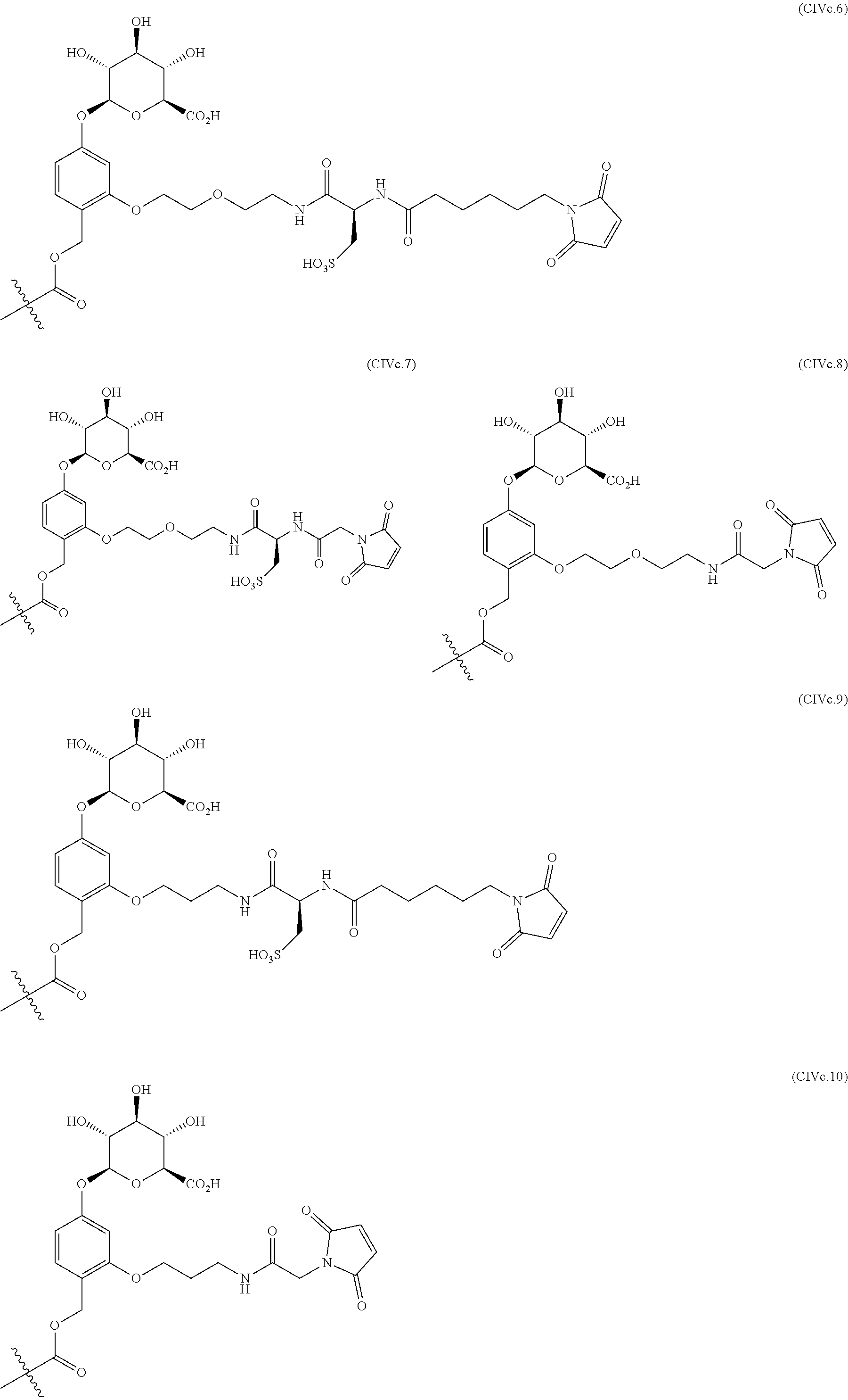

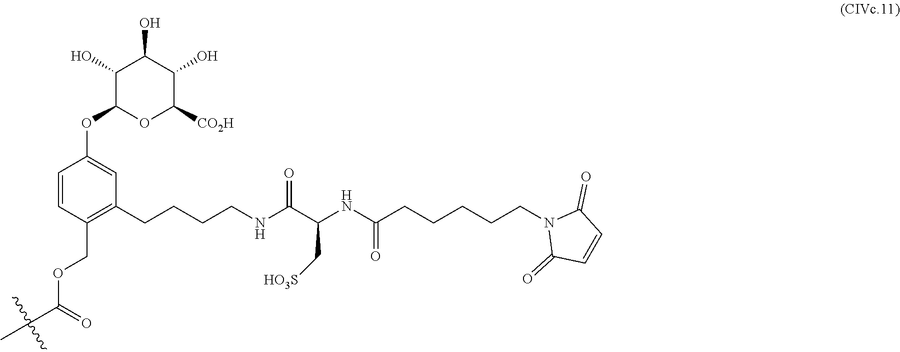

wherein [symbol] represents the point of attachment of the linker (L) to a compound of the present disclosure, or pharmaceutically acceptable isomer, tautomer, racemate, hydrate, solvate, isotope, or salt thereof.

Exemplary embodiments of linkers according to structural formula (CIVd) that may be included in the conjugates include the linkers illustrated below (as illustrated, the linkers include a group suitable for covalently linking the linker to a binding protein of this disclosure):

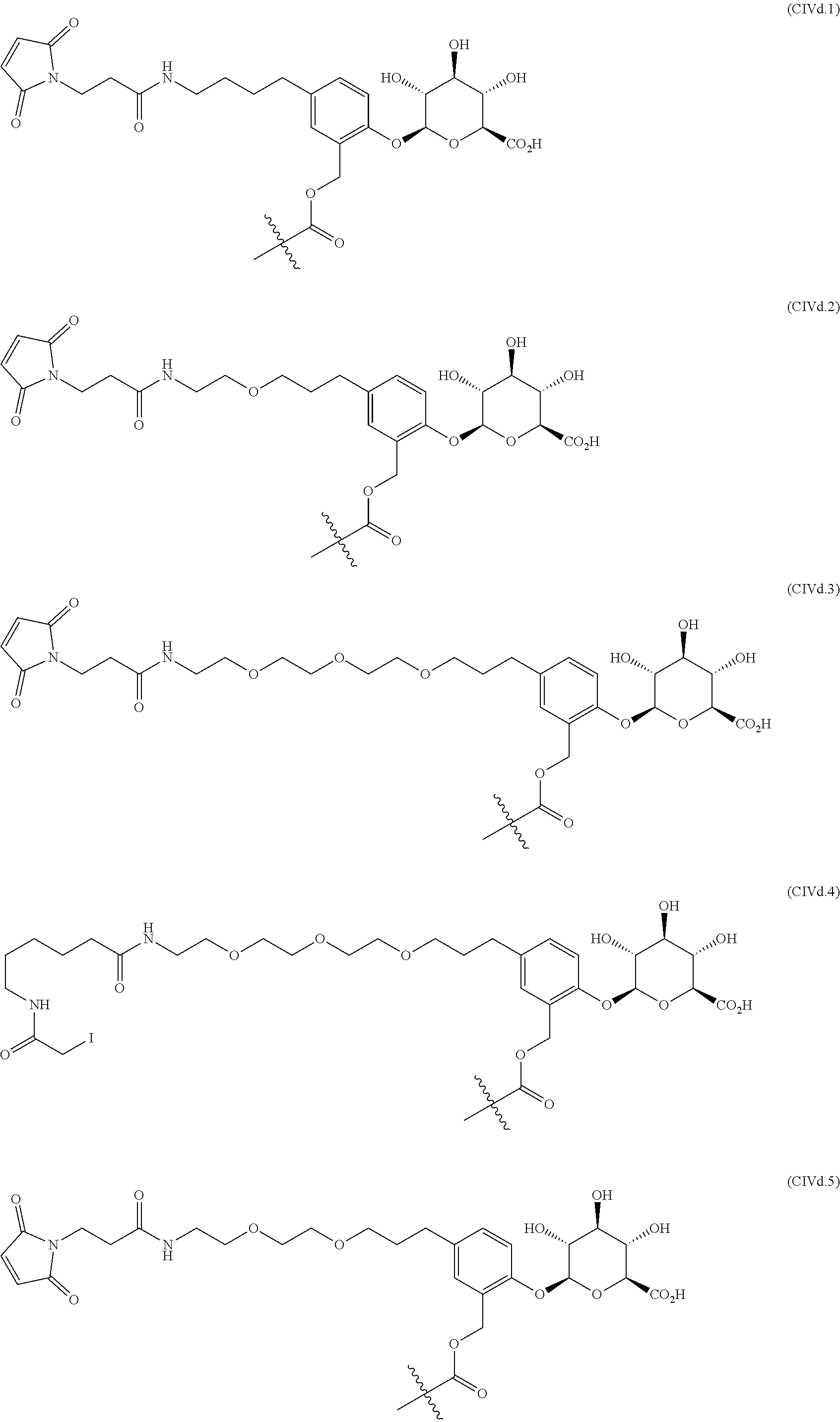

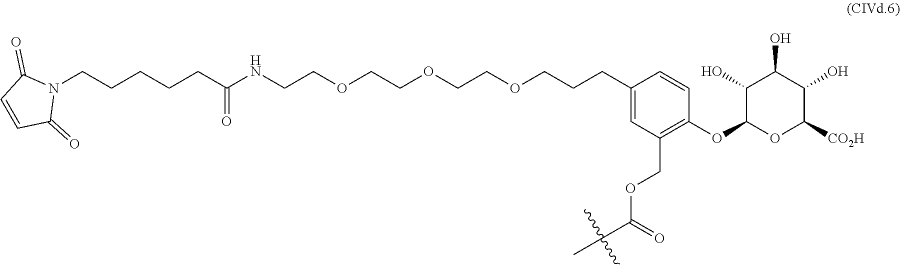

wherein [symbol] represents the point of attachment of the linker (L) to a compound of the present disclosure, or pharmaceutically acceptable isomer, tautomer, racemate, hydrate, solvate, isotope, or salt thereof.

Exemplary embodiments of linkers according to structural formula (CIVe) that may be included in the conjugates include the linkers illustrated below (as illustrated, the linkers include a group suitable for covalently linking the linker to a binding protein of this disclosure):

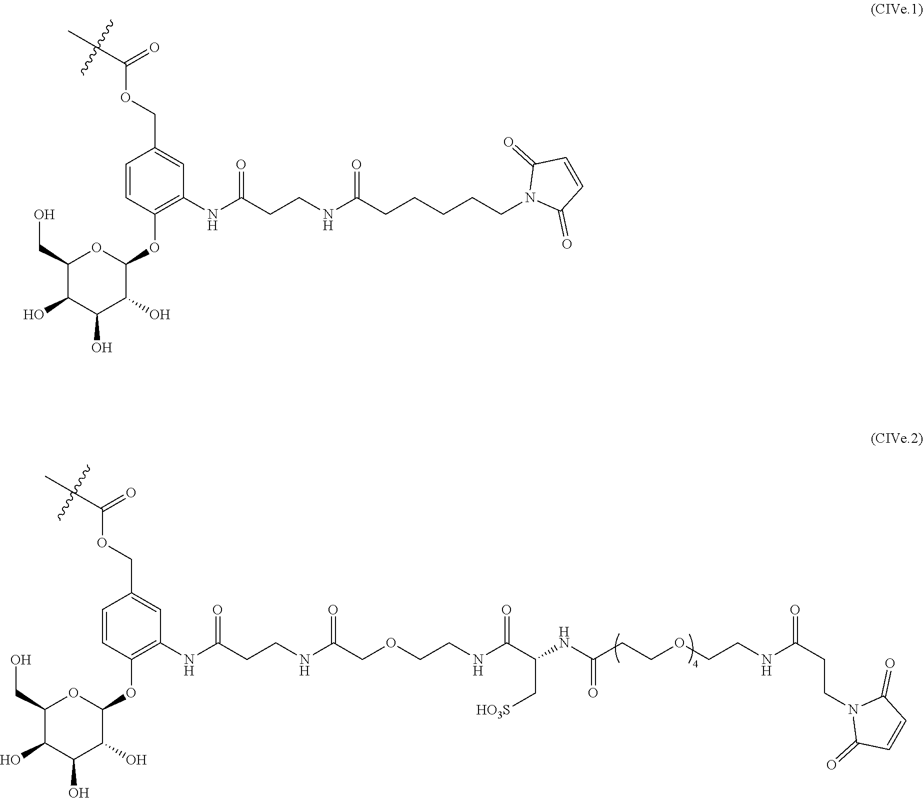

wherein [symbol] represents the point of attachment of the linker (L) to a compound of the present disclosure, or pharmaceutically acceptable isomer, tautomer, racemate, hydrate, solvate, isotope, or salt thereof.

Although cleavable linkers can provide certain advantages, the linkers comprising the conjugate need not be cleavable. For non-cleavable linkers, the payload compound release may not depend on the differential properties between the plasma and some cytoplasmic compartments. The release of the payload compound can occur after internalization of the conjugate via antigen-mediated endocytosis and delivery to lysosomal compartment, where a binding protein of this disclosure can be degraded to the level of amino acids through intracellular proteolytic degradation. This process can release a payload compound derivative (a metabolite of the conjugate containing a non-cleavable linker-heterocyclic compound), which is formed by the payload compound, the linker, and the amino acid residue or residues to which the linker was covalently attached. The payload compound derivative from conjugates with non-cleavable linkers can be more hydrophilic and less membrane permeable, which can lead to less bystander effects and less nonspecific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols or amide polymers. The linker can contain a polyethylene glycol segment having from 1 to 6 ethylene glycol units.

The linker can be non-cleavable in vivo, for example, a linker-payload comprising a compound of the present disclosure, or pharmaceutically acceptable isomer, tautomer, racemate, hydrate, solvate, isotope, or salt thereof, and a linker L; -L is represented by the formulas below:

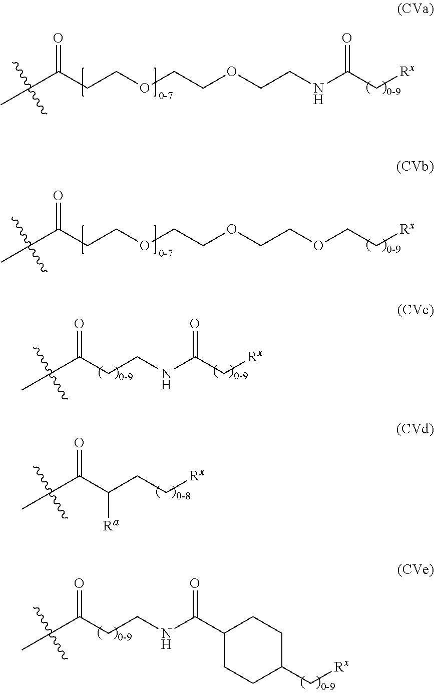

or salts thereof, wherein: R^{a} is selected from hydrogen, alkyl, sulfonate and methyl sulfonate; RX is a reactive moiety including a functional group capable of covalently linking the linker to a binding protein of this disclosure; and [symbol] represents the point of attachment of the linker (L) to a compound of the present disclosure, or pharmaceutically acceptable isomer, tautomer, racemate, hydrate, solvate, isotope, or salt thereof.

In some embodiments, for a linker-payload comprising a compound of the present disclosure, or pharmaceutically acceptable isomer, tautomer, racemate, hydrate, solvate, isotope, or salt thereof, and a linker L; -L is represented by the formula:

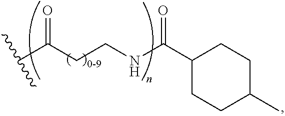

wherein n=0-9 and [symbol] represents the point of attachment to the compound (payload).

In some embodiments, for a linker-payload comprising a compound of the present disclosure, or pharmaceutically acceptable isomer, tautomer, racemate, hydrate, solvate, isotope, or salt thereof, and a linker L; -L is represented by the formula:

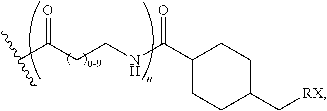

wherein RX comprises a reactive moiety, e.g., a maleimide or a leaving group, n=0-9, and [symbol] represents the point of attachment to the compound (payload).

In some embodiments, for a conjugate comprising a compound of the present disclosure, or pharmaceutically acceptable isomer, tautomer, racemate, hydrate, solvate, isotope, or salt thereof, a linker L, and a binding protein of this disclosure; -L- is represented by the formula:

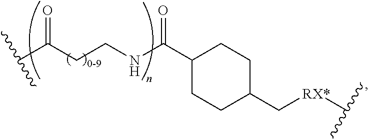

RX* is a bond, a succinimide moiety, or a hydrolyzed succinimide moiety attached at the [symbol] on the right to a residue of the binding protein of this disclosure, [symbol] on the left represents the point of attachment to the compound (payload), and n=0-9.

Exemplary embodiments of linkers according to structural formula (CVa)-(Ve) that may be included in the conjugates include the linkers illustrated below (as illustrated, the linkers include a group suitable for covalently linking the linker to a binding protein of this disclosure, and [symbol] represents the point of attachment of the linker (L) to a compound of the present disclosure, or pharmaceutically acceptable isomer, tautomer, racemate, hydrate, solvate, isotope, or salt thereof:

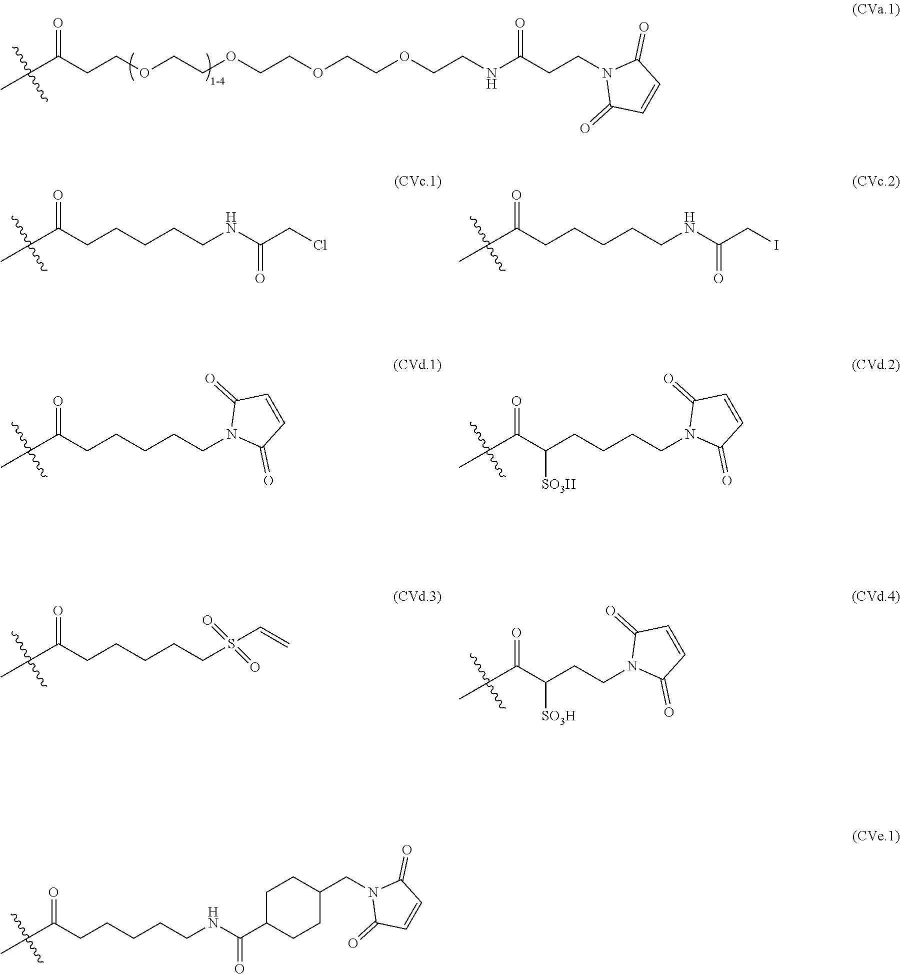

Attachment groups that are used to attach the linkers to a binding protein of this disclosure can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used with GR agonist compounds of the disclosure. Examples of cysteine-based linkers are provided in PCT Patent Application Publication Number WO 2020/092385, the linkers of which are incorporated by reference herein.

Maleimide groups are frequently used in the preparation of conjugates because of their specificity for reacting with thiol groups of, for example, cysteine groups of a binding protein of this disclosure. The reaction between a thiol group of a binding protein of this disclosure and a drug with a linker (linker-payload) including a maleimide group proceeds according to the following scheme:

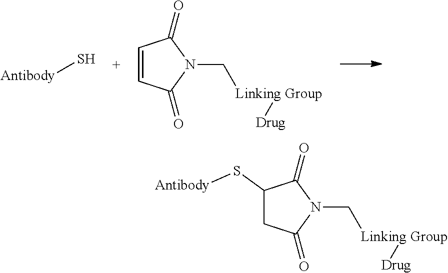

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linking group shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

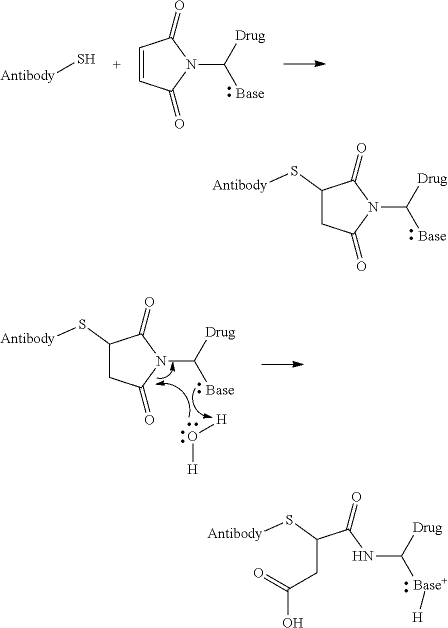

The hydrolysis reaction schematically represented above may occur at either carbonyl group of the succinimide group. Accordingly, two possible isomers may result, as shown below:

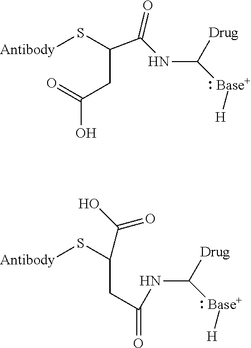

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker, e.g., any L with a maleimide group prior to conjugation to a binding protein of this disclosure may facilitate hydrolysis of a nearby succinimide group formed after conjugation of the binding protein of this disclosure to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH2)ₘ, where m is an integer from 0 to 10. An alkylene chain may be optionally substituted with other functional groups of the disclosure.

A linker (L) with a maleimide group may include an electron withdrawing groups, such as —C(O)R, ═O, —CN, —NO₂, —CX₃, —X, —C(O)OR, —C(O)NR₂, —C(O)R, —C(O)X, —SO₂R, —SO₂OR, —SO₂NHR, —SO₂NR₂, PO₃R₂, —P(O)(CH₃)NHR, —NO, —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups, such as those of the disclosure.

Examples of self-stabilizing linkers are provided in, e.g., U.S. Patent Application Publication Number US 2013/0309256, the linkers of which are incorporated by reference herein. It will be understood that a self-stabilizing linker useful in conjunction with the compounds of the present disclosure may be equivalently described as unsubstituted maleimide-including linkers, thio-substituted succinimide-including linkers, or hydrolyzed, ring-opened thio-substituted succinimide-including linkers.

In some embodiments, for a linker-payload comprising a compound of the present disclosure, or pharmaceutically acceptable isomer, tautomer, racemate, hydrate, solvate, isotope, or salt thereof, and a linker L; -L comprises a self-stabilizing moiety. For example, L may be selected from:

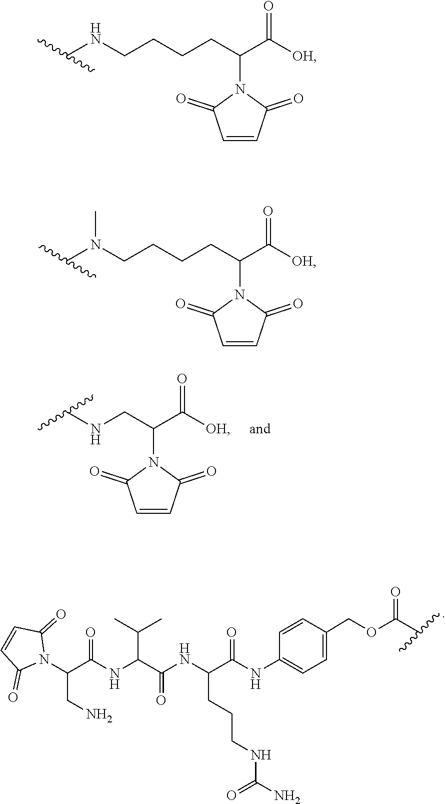 [SMILES: *NCCCCC(C(=O)O)N1C(=O)C=CC1=O ; *N(C)CCCCC(C(=O)O)N1C(=O)C=CC1=O ; *NCC(C(=O)O)N1C(=O)C=CC1=O ; "and" ; *C(=O)OCc1ccc(NC(=O)C(CCCNC(N)=O)NC(=O)C(NC(=O)C(CN)N2C(=O)C=CC2=O)C(C)C)cc1]

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represent the point of attachment to a compound of any one of Structure (Iq) or (IIq) or pharmaceutically acceptable isomer, tautomer, racemate, hydrate, solvate, isotope, or salt thereof.

A method for bridging a pair of sulfhydryl groups derived from reduction of a native hinge disulfide bond has been disclosed and is depicted in the schematic below. An advantage of this methodology is the ability to synthesize homogenous conjugates by full reduction of IgGs (to give 4 pairs of sulfhydryls from interchain disulfides, wherein the DAR can range from 1 to 8) followed by reaction with 4 equivalents of the alkylating agent. Conjugates containing “bridged disulfides” are also claimed to have increased stability.

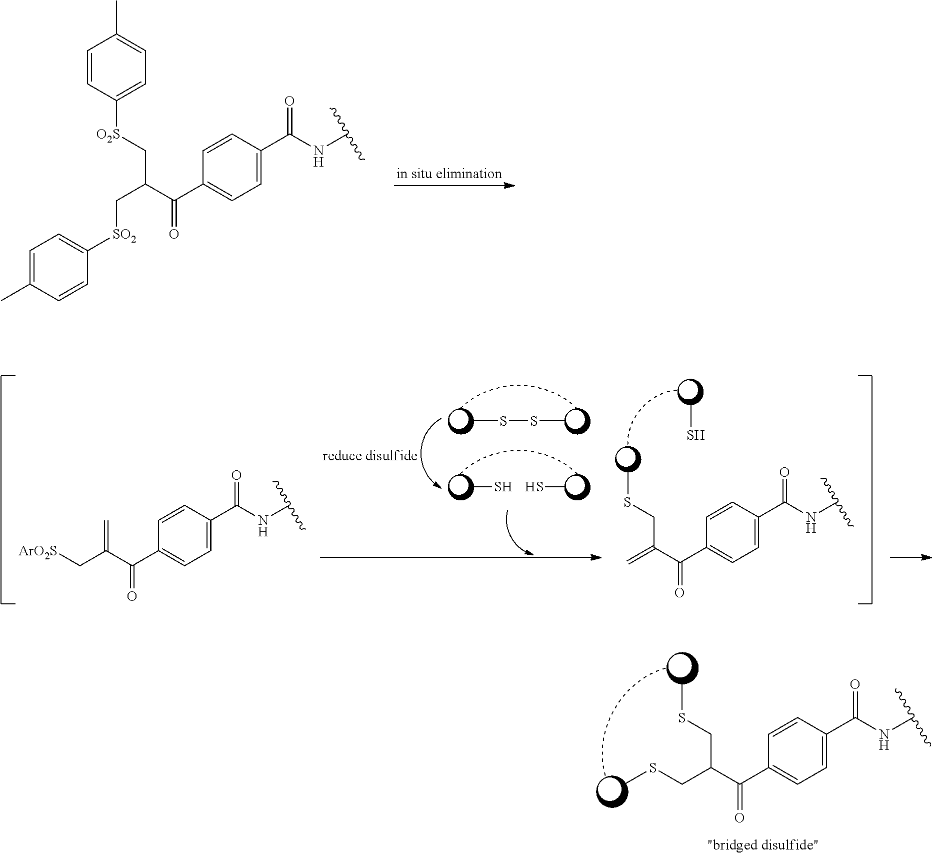

Similarly, as depicted below, a maleimide derivative that is capable of bridging a pair of sulfhydryl groups has been developed.

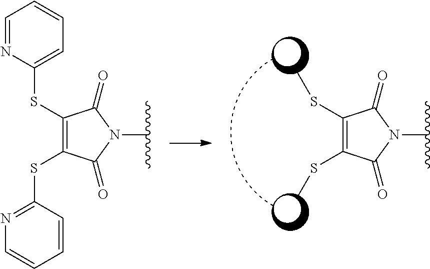 [SMILES: *N1C(=O)C(Sc2ccccn2)=C(Sc2ccccn2)C1=O ; *N1C(=O)C(SC)=C(SC)C1=O]

A linker of the disclosure, L, can contain the following structural formulas (CVIa), (CVIb), or (CVIc):

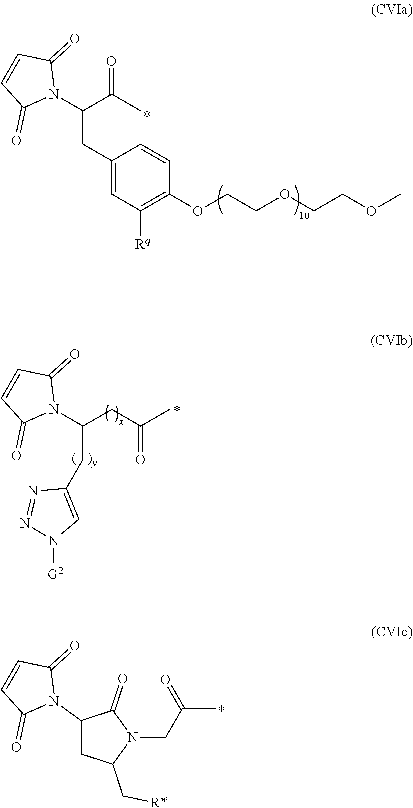

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R_{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (CVIa) and (CVIb), which can be included in linker-payload and conjugate structures of this disclosure, include the linkers illustrated below (as illustrated, the linkers include a group suitable for covalently linking the linker to an antibody construct):

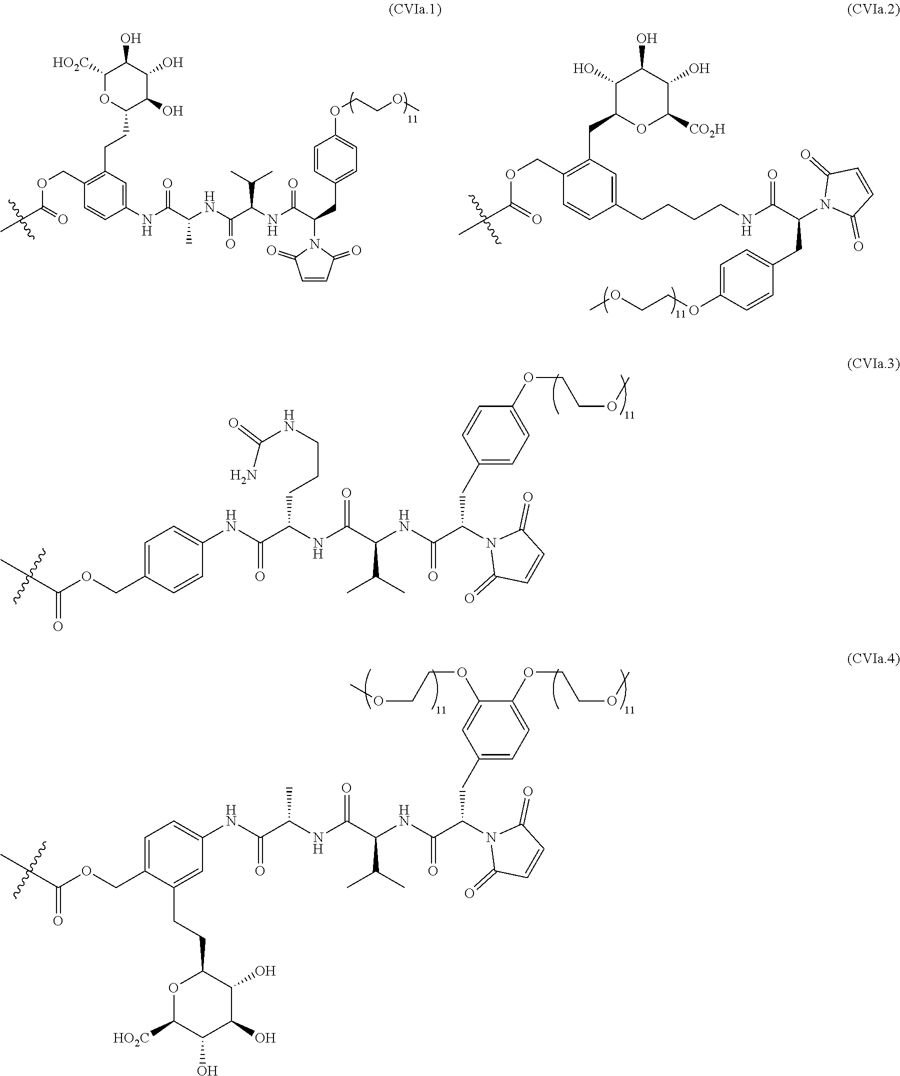

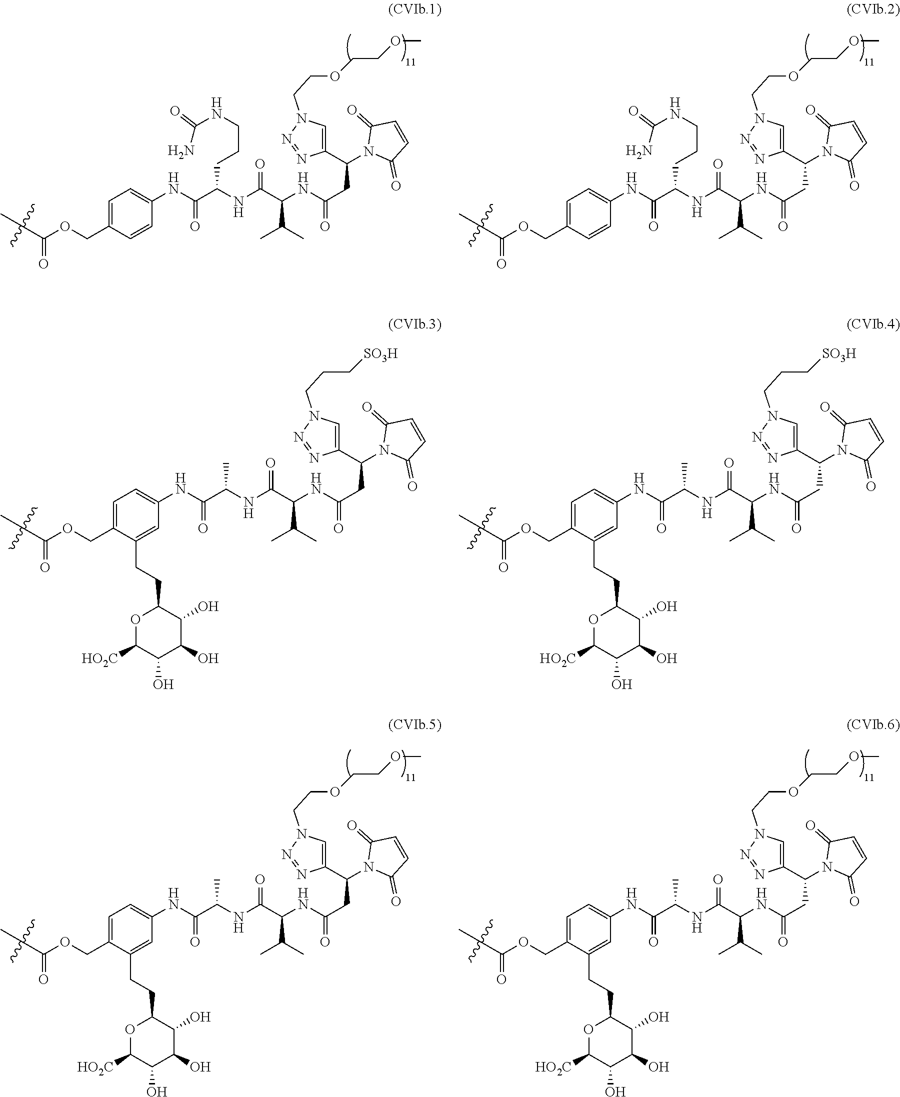

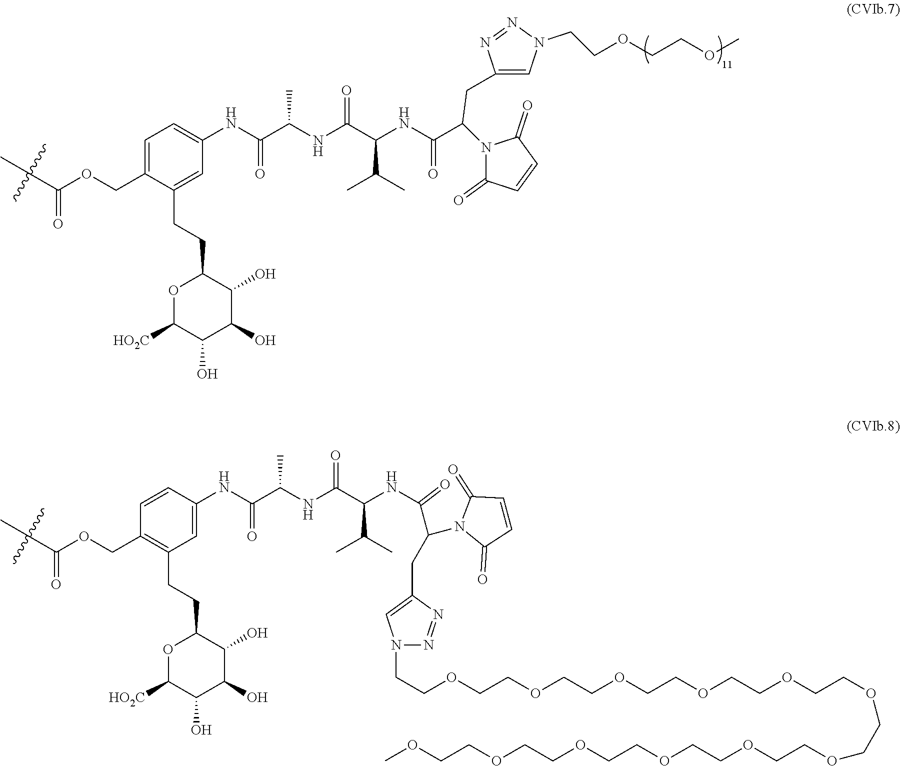

wherein [symbol] represents the point of attachment of the linker (L) to a compound of the present disclosure, or pharmaceutically acceptable isomer, tautomer, racemate, hydrate, solvate, isotope, or salt thereof.

Exemplary embodiments of linkers according to structural formula (CVIc), which can be included in linker-payload and conjugate structure of this disclosure, include the linkers illustrated below (as illustrated, the linkers include a group suitable for covalently linking the linker to a binding protein of this disclosure):

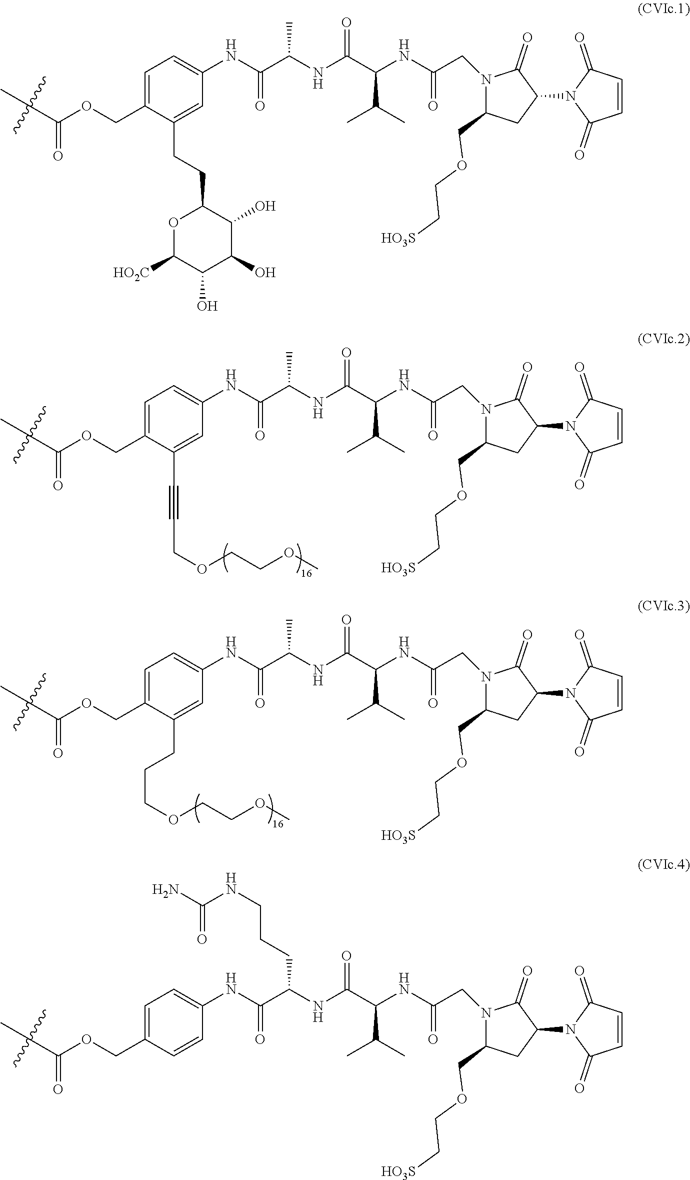

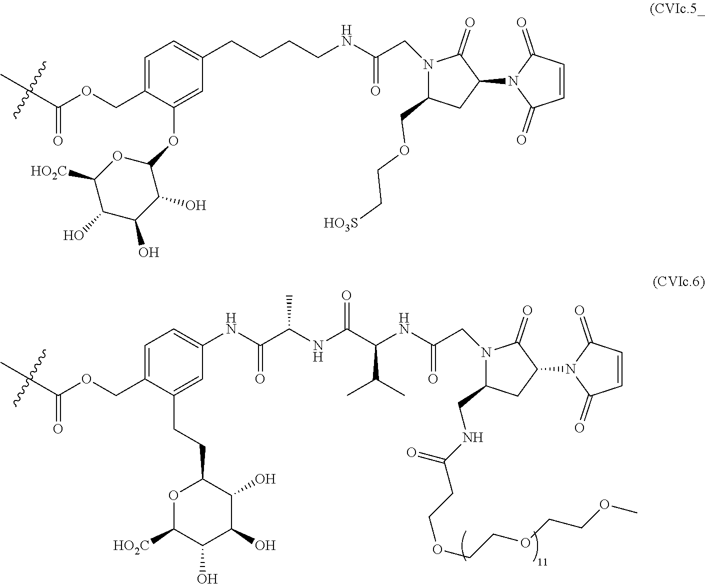

wherein [symbol] represents the point of attachment of the linker (L) to a compound of the present disclosure, or pharmaceutically acceptable isomer, tautomer, racemate, hydrate, solvate, isotope, or salt thereof.

Some exemplary linkers (L) are described in the following paragraphs. In some embodiments for a compound of the present disclosure, or pharmaceutically acceptable isomer, tautomer, racemate, hydrate, solvate, isotope, or salt thereof, wherein attachment of the linker is to a nitrogen of the compound and conjugation is to a cysteine residue of an antibody or targeting moiety, -L is represented by the formulas set forth in Table 3 below:

TABLE 3
Exemplary Linkers Targeting Cysteine

| Linker | |
|---|---|
| Name | Structural Formula |
| L1 | 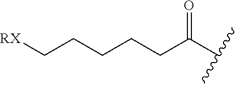 |
| L2 | 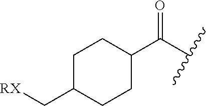 |
| L3 | 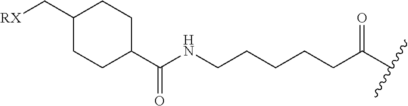 |
| L4 | 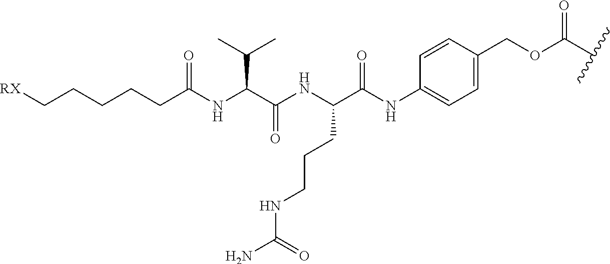 |
| L5 | 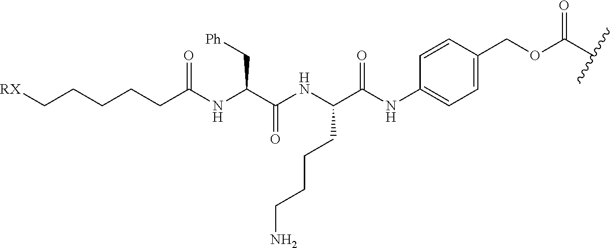 |
| L6 | 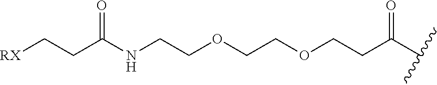 |
| L7 | 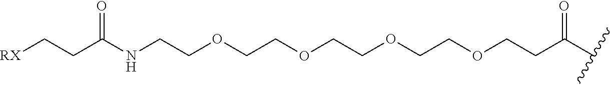 |
| L8 | 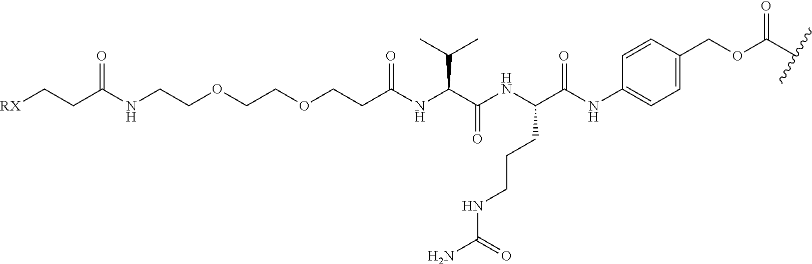 |
| L9 | 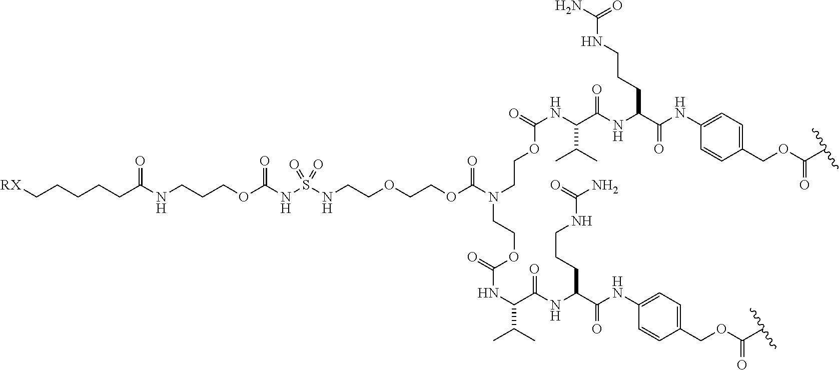 |
| L10 | 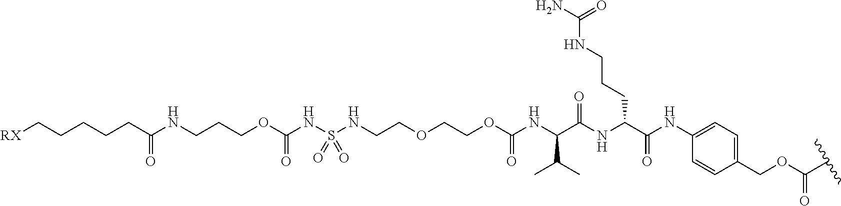 |
| L11 | 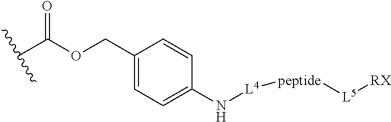 |

wherein

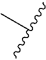

represents attachment to a nitrogen of a compound or salt of the present disclosure; L⁴ represents the C-terminus of the peptide and L⁵ is selected from a bond, alkylene and heteroalkylene, wherein L⁵ is optionally substituted with one or more groups independently selected from R³⁰, and R³⁰ is independently selected at each occurrence from halogen, —OH, —CN, —O-alkyl, —SH, ═O, ═S, —S(O)₂OH, —NH₂, —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —S(O)₂OH, —NH₂, and —NO₂; and RX represents a reactive moiety. The reactive moiety may be selected, for example, from an electrophile, e.g., an α,β-unsaturated carbonyl, such as a maleimide, and a leaving group. In certain embodiments, RX of any one of linkers L1 to L11 is a maleimide. In certain further embodiments, RX is

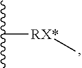

wherein RX* is a bond, a succinimide moiety, or a hydrolyzed succinimide moiety bound to a cysteine residue of an antibody, an antibody construct or a targeting moiety, wherein

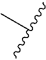

on RX* represents the point of attachment to such residue.

In some embodiments for a compound of the present disclosure, or pharmaceutically acceptable isomer, tautomer, racemate, hydrate, solvate, isotope, or salt thereof, attachment of the linker is to a nitrogen of a compound of the present disclosure, or pharmaceutically acceptable isomer, tautomer, racemate, hydrate, solvate, isotope, or salt thereof, and conjugation is to a lysine residue of a binding protein of this disclosure. In certain embodiments, -L is represented by the formulas set forth in Table 4 below.

TABLE 4
Exemplary Linkers Targeting Lysine

| Linker | |
|---|---|
| Name | Structural Formula |
| L38 | 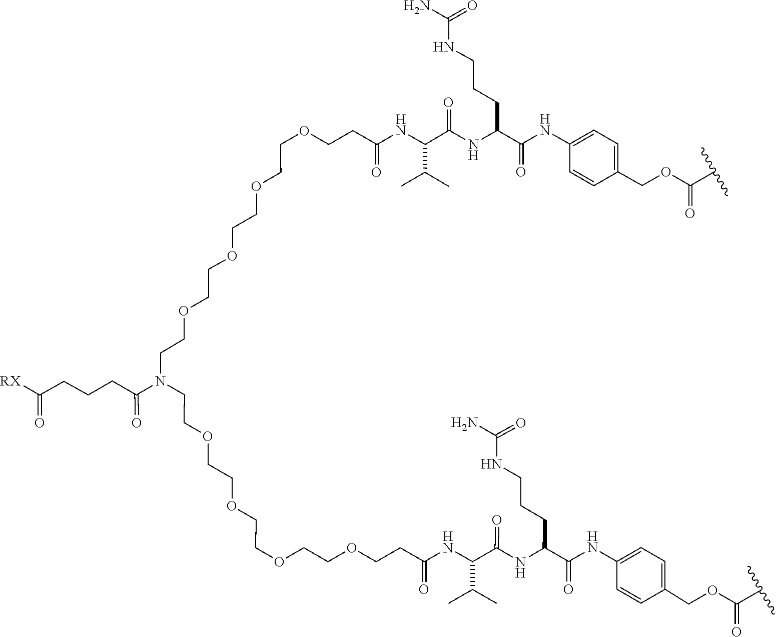 |
| L39 | 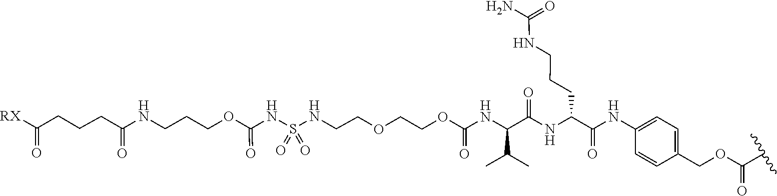 |

wherein

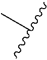

represents attachment to a nitrogen of a compound of the present disclosure, or pharmaceutically acceptable isomer, tautomer, racemate, hydrate, solvate, isotope, or salt thereof, and RX represents a reactive moiety. In certain embodiments, RX of linker L12 or L13 is a maleimide. In certain further embodiments, RX is

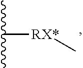

wherein RX* is a bond, a succinimide moiety, or a hydrolyzed succinimide moiety bound to a lysine residue of an antibody, an antibody construct, or a targeting moiety, wherein

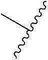

on RX* represents the point of attachment to such residue.

As is known by skilled artisans, the linker selected for a particular conjugate may be influenced by a variety of factors, including the site of attachment to a binding protein of this disclosure, lysine, cysteine, or other amino acid residues, structural constraints of the drug pharmacophore, and the lipophilicity of the drug. The specific linker selected for a conjugate should seek to balance these different factors for the polypeptide comprising the binding domain (e.g., a fusion protein, or an antibody or an antigen-binding fragment thereof) and drug combination.

For example, cytotoxic conjugates have been observed to effect killing of bystander antigen-negative cells present in the vicinity of the antigen-positive tumor cells. The mechanism of the bystander effect by cytotoxic conjugates has indicated that metabolic products formed during intracellular processing of the conjugates may play a role. Neutral cytotoxic metabolites generated by metabolism of the conjugates in antigen-positive cells appear to play a role in bystander cell killing while charged metabolites may be prevented from diffusing across the membrane into the medium, or from the medium across the membrane and, therefore, cannot affect cell killing via the bystander effect. In some embodiments, a linker is selected to attenuate the bystander effect caused by cellular metabolites of the conjugate. In further embodiments, a linker is selected to increase the bystander effect.

The properties of the linker, or linker-payload, may also impact aggregation of a conjugate under conditions of use or storage. Generally, conjugates reported in the literature contain about 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the payload compound is more hydrophobic in nature, it may be desirable to select linkers that are relatively hydrophilic as a means of reducing conjugate aggregation, especially in instances where DARs greater than 3-4 are desired. Thus, in some embodiments, a linker incorporates chemical moieties that reduce aggregation of the conjugates during storage or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In preferred embodiments, aggregation of conjugates during storage or use is less than about 40% as determined by size-exclusion chromatography (SEC). In particular embodiments, the aggregation of the conjugates during storage or use is less than about 35%, such as less than about 30%, about 25%, about 20%, about 15%, about 10%, about 5%, about 4%, or even less, as determined by size-exclusion chromatography (SEC).

2. Category II Linkers

Other linkers useful in various embodiments include Category II linkers. In certain embodiments, a conjugate comprises a binding protein of this disclosure wherein the linker is a Category II linker linking the GR agonist to the polypeptide comprising segments, e.g., a spacer which does not affect the binding of the active portions of the conjugate, i.e., the antigen binding domains or in the release of the drug compound.

In another embodiment, the conjugate includes a Category II linker and is represented by formula (LII-1):

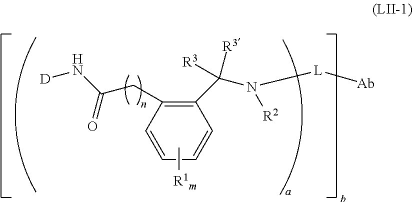

or a pharmaceutically acceptable salt thereof, wherein:
a is an integer from 1 to 20; b is an integer from 1 to 20; m is 0, 1, 2, 3, or 4; n is 0 or 1; D-NH— is a compound of Categories A to K, wherein one H has been replaced with a covalent bond to the —C(═O)X— group; each R¹ is independently selected from C₁-C₄alkyl, O—C₁-C₄alkyl, and halogen; R² is selected from C₁-C₄alkyl and —(CH₂CH₂O)s-CH₃; wherein s is an integer from 1 to 10; R³ and R^{3′} are each independently selected from hydrogen and C₁-C₃alkyl; L is a cleavable linker; and Ab is a binding protein of this disclosure.

In another embodiment of LIT-1, a is an integer from 1 to 4; b is an integer from 1 to 10; and m is 0. In yet another embodiment, a is an integer from 1 to 4; m is 0; n is 0; and R³ and R^{3′} are each hydrogen. In some embodiments, the linker L is a Category II represented by formula (LII-2):

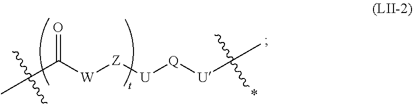

wherein [symbol] is the point of attachment to a nitrogen atom of a compound of Structure I or II; [symbol] is the point of attachment to Ab; t is an integer from 1 and 10; W is absent or a self-immolative moiety;
Z is absent or a peptide of 2 to 5 amino acids; U and U′ are independently absent or a spacer; and
Q is a heterobifunctional group; provided that W and Z are not both absent.

In some embodiments, W is a self-immolative moiety segment in the linker of group L^{II}. In some embodiments, W is a group of its own and is selected from:

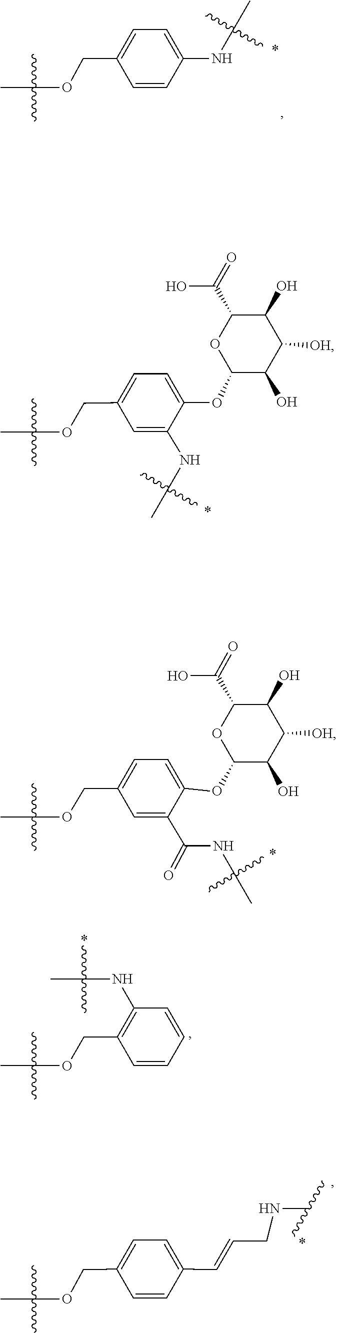

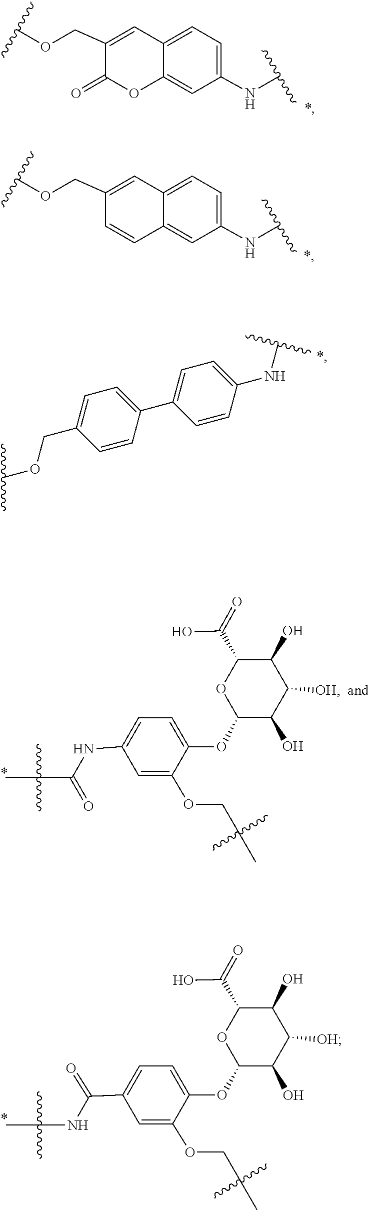

wherein:
[symbol] the point of attachment to the carbonyl group; and [symbol] is the point of attachment to Z.

In other embodiments, W is selected from

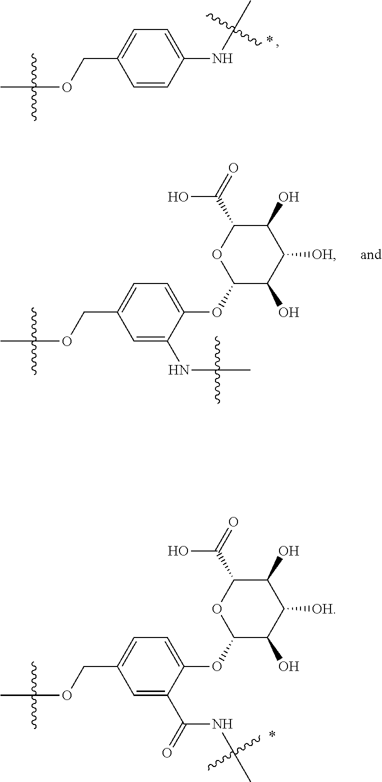 [SMILES: *Nc1ccc(CO*)cc1 ; *Nc1cc(CO*)ccc1O[C@@H]1O[C@H](C(=O)O)[C@@H](O)[C@H](O)[C@H]1O ; *NC(=O)c1cc(CO*)ccc1O[C@@H]1O[C@H](C(=O)O)[C@@H](O)[C@H](O)[C@H]1O]

In some embodiments, W is

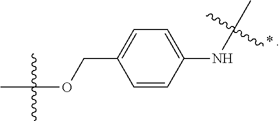 [SMILES: *Nc1ccc(CO*)cc1]

In further embodiments, Z in linker LII-2 comprises a peptide capable of being enzymatically cleaved. In still further embodiments, Z is a cathepsin cleavable group of peptides.

In some embodiments, Z is a two-amino acid peptide selected from Val-Cit, Cit-Val, Val-Ala, Ala-Val, Phe-Lys, and Lys-Phe. In one embodiment, Z is Val-Ala or Ala-Val. In certain embodiments, U and U′ are permutable conjugates in linker LII-2. In some embodiments of linker LII-2, U and U′ are independently absent or selected from

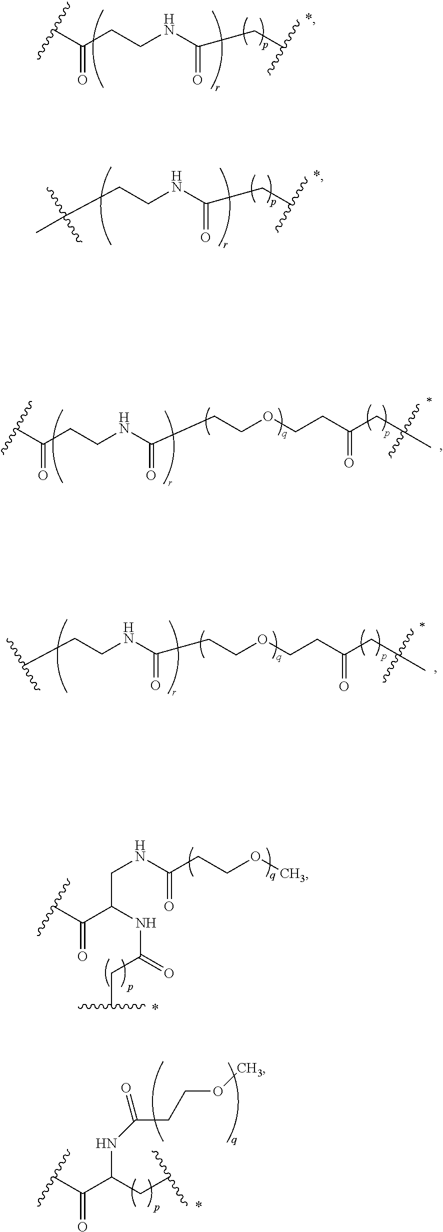

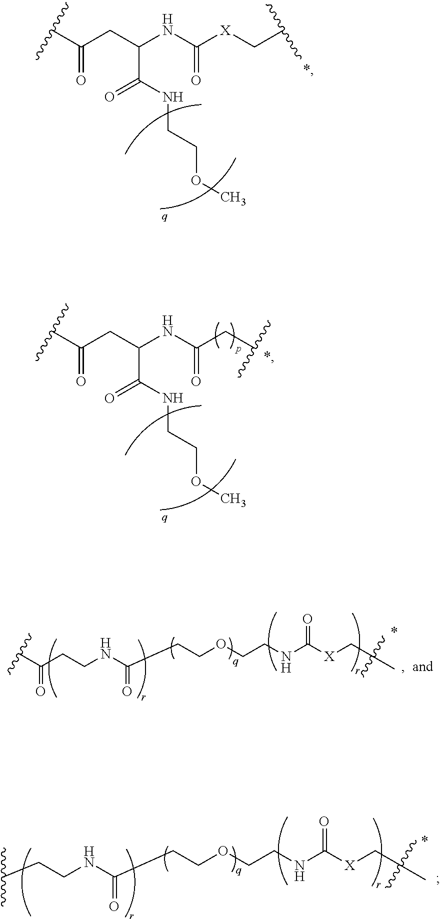

wherein
[symbol] is the point of attachment to Z; [symbol] is the point of attachment to Q; p is an integer from 1 to 6; q is an integer from 1 to 20; X is O or —CH₂—; and each r is independently 0 or 1.

In one embodiment, U′ is absent and U is represented by formula (LII-3)

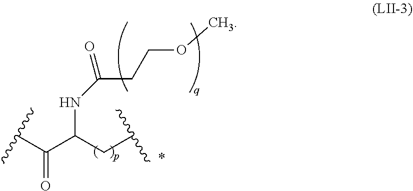

In some embodiments, Q is a permutable conjugate in linker LII-2. In other embodiments, Q is a heterobifunctional group or RG which is capable of being attached to Ab through chemical or enzyme-mediated conjugation. In further embodiments, Q is selected from

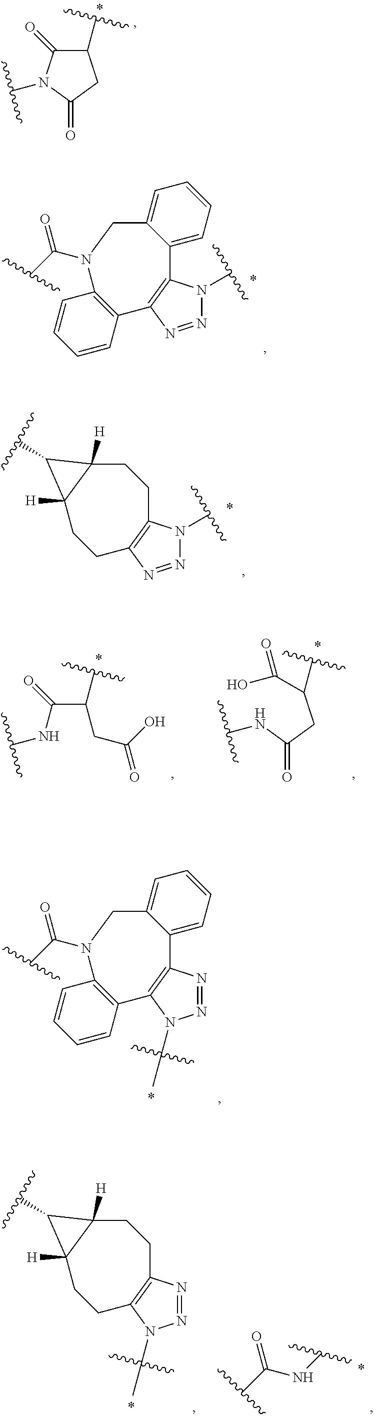

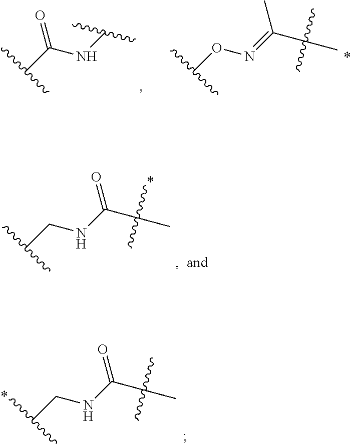

wherein [symbol] is the point of attachment to U or, when U is absent, the point of attachment to Z; and [symbol] is the point of attachment to U′, or, when U′ is absent, the point of attachment to Ab.

In some embodiments, a GR agonists of Categories A to K, or a pharmaceutically acceptable salt thereof, is linked to a binding protein of this disclosure, via a linker LII-4:

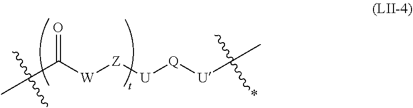

and wherein t is 1; W is absent or a self-immolative group; and Z is absent or a peptide of two amino acids.

In another embodiment, the conjugate of the present disclosure is represented by formula (LII-5):

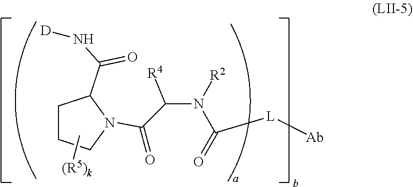

or a pharmaceutically acceptable salt thereof, wherein:
a is an integer from 1 to 20; b is an integer from 1 to 20; k is 0, 1, 2, or 3; m is 0, 1, 2, 3, or 4; D-NH— is a compound of the present invention, wherein one H has been replaced with a covalent bond to the —C(═O)X— group; R² is selected from H, C₁-C₄alkyl and —(CH₂CH₂O)ₛ—CH₃, wherein s is an integer from 1 and 10; R⁴ is selected from hydrogen and any naturally occurring amino acid side chain; R⁵ is selected from C₁-C₄alkyl, and O—C₁-C₄alkyl; L is a cleavable linker; and
Ab is a binding protein of this disclosure.

In another embodiment Category II linkers are represented by formula (LII-6), wherein:

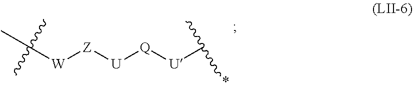

[symbol] is the point of attachment to the carbonyl group; [symbol] is the point of attachment to Ab; W is a self-immolative moiety; Z is absent or a peptide of 2 to 5 amino acids; and U and U′ are independently absent or a spacer; and Q is a heterobifunctional group.

In another embodiment, a conjugate of the present disclosure is represented by formula (LII-7):

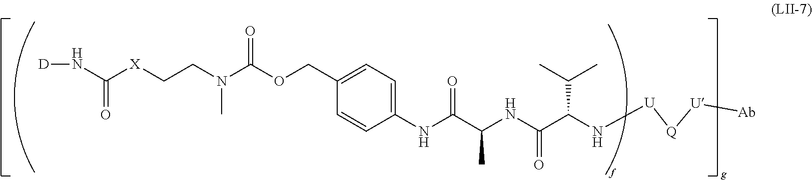

or a pharmaceutically acceptable salt thereof, wherein:
D-NH— is a compound of Categories A to K, wherein one H has been replaced with a covalent bond to the —C(═O)X— group.

In one embodiment, the LII-2 type is represented by formula (LII-8-LII-10):

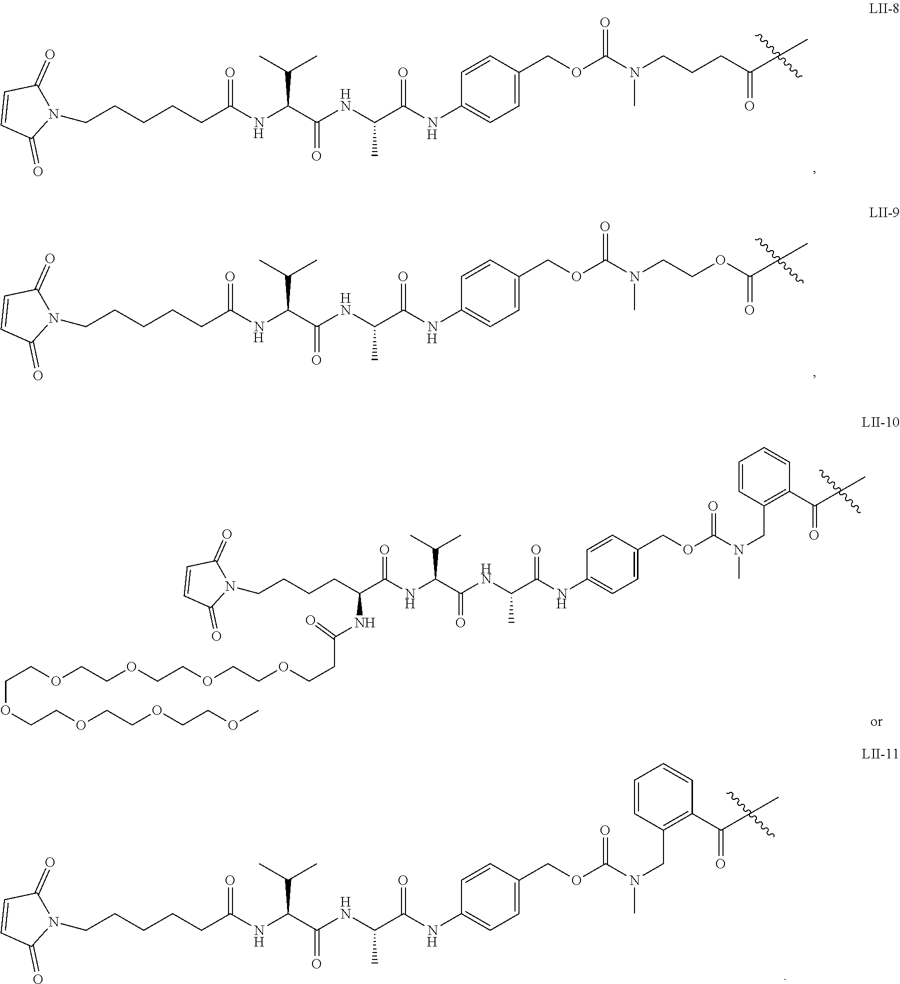

wherein [symbol] is the point of attachment to an amino group on a compound of the present invention.

In one embodiment, the term “self-immolative moiety” or “self-immolative group” refers to a functional group that undergoes an electronic cascade which results in the release of the moiety, functional group, or molecule to which it is attached. In some embodiments, the self-immolative group comprises one or more groups which can undergo 1,4-elimination, 1,6-elimination, 1,8-elimination, 1,6-cyclization elimination, 1,5-cyclization elimination, 1,3-cyclization elimination, intramolecular 5-exo-trig cyclization, or 6-exo-trig cyclization. In some embodiments the self-immolative group can be any of those disclosed in PCT publications WO 2018/200812 and WO 2018/100558, which moieties are incorporated by reference herein in their entireties.

In one embodiment the group “Z” in Category II linkers is absent or a peptide of 2 to 5 amino acids. In some embodiments, the peptide is the site of cleavage of the linker, thereby facilitating release of the drug upon exposure to intracellular proteases, such as lysosomal enzymes (Doronina et al. (2003) Nat. Biotechnol. 21:778-784). Examples of peptides having two amino acids include alanine-alanine (ala-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Examples of peptides having three amino acids include glycine-valine-citrulline (gly-val-cit) and glycine-glycine-glycine (gly-gly-gly). The amino acid combinations above can also be present in the reverse order (i.e., cit-val).

The peptides of the present disclosure may comprise naturally-occurring or non-natural amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, and Tyr. “Non-natural amino acids” include homoserine, homoarginine, citrulline, phenylglycine, taurine, iodotyrosine, selenocysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor-associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

The groups “U” and “U” in Category II linkers are independently absent or a spacer. As used herein, the term “spacer,” refers to chemical moiety that serves as a connector. In the present disclosure the spacer can connect the binding protein of this disclosure to the heterobifunctional group or connect the heterobifunctional group to peptide “Z,” or, when “Z” is absent, to group “W”. Non-limiting exemplary spacers include —NH—, —S—, —O—, —NHC(═O)CH₂CH₂—, —S(═O)₂—CH₂CH₂—, —C(═O)NHNH—, —C(═O)O—, —C(═O)NH—, —CH₂—, —CH₂CH₂—, —CH₂CH₂CH₂—, —CH₂═CH₂—, —C^{o}C—, —CH═N—O—, polyethylene glycol (PEG),

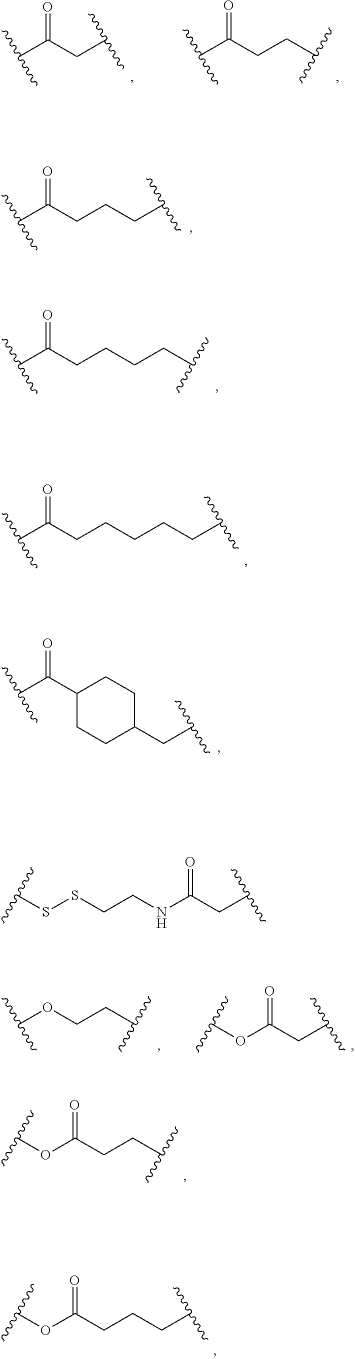

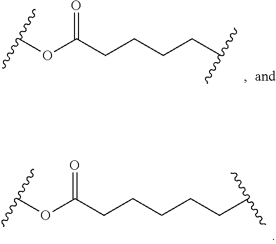

In the compounds having Category II linkers, when “U” is present, it can be a branched group substituted by from 1 to 10 “—C(O)—W—Z—” groups. In some embodiments, “U” is substituted by from 1 to 5“—C(O)—W—Z—” groups. In some embodiments, “U” is substituted with 1 or 2 “—C(O)—W—Z—” groups. In some embodiments, “U” is substituted with 1 “—C(O)—W—Z—” group. In some embodiments the spacer can be any of those disclosed in PCT publications WO 2018/200812, WO 2018/100558, which are incorporated by reference in their entireties.

Group “Q” as defined for compounds with a linker of Category II, is a heterobifunctional or reactive group (RG). In the present disclosure, the term “heterobifunctional group” refers to a chemical moiety that connects the linker of which it is a part to the binding protein of this disclosure. See, e.g., WO2017/191579. Heterobifunctional groups are characterized as having different reactive groups at either end of the chemical moiety. The heterobifunctional group may be attached directly to “Ab,” or alternatively, may connect through linker “U”. Attachment to “Ab,” can be accomplished through chemical or enzymatic conjugation, or a combination of both. Chemical conjugation involves the controlled reaction of accessible amino acid residues on the surface of the polypeptide comprising a binding domain with a reaction handle on “Q” or “U”. Examples of chemical conjugation include lysine amide coupling, cysteine coupling, and coupling via a non-natural amino acid incorporated by genetic engineering, wherein non-natural amino acid residues with a desired reaction handle are installed onto “Ab”. In enzymatic conjugation, an enzyme mediates the coupling of the linker with an accessible amino residue on the binding protein of this disclosure. Examples of enzymatic conjugation include transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Ab” to be utilized in subsequent chemical conjugation. In some embodiments the heterobifunctional group can be any of those disclosed in PCT publications WO 2018/200812, WO 2018/100558, which are incorporated by reference in their entireties.

3. Category III Linkers

In some other aspects, the present disclosure relates to a conjugate comprising a compound of Categories A to K linked to a binding protein of this disclosure, via a Category III linker, wherein the conjugate has Formula (LIII-I):

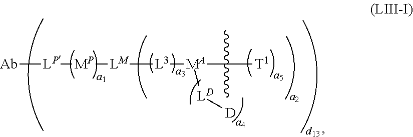

wherein:
Ab is a binding protein comprising a binding domain (e.g., a fusion protein, or an antibody or an antigen-binding fragment thereof); a₁, when present, is an integer from 0 to 1; a₂ is an integer from 1 to 3; a₃, when present, is an integer from 0 to 1; a₄ is an integer from 1 to about 5; a₅ is an integer from 1 to 3; d₁₃ is an integer from 1 to about 6; L^{p′} is a divalent linker moiety connecting the antibody to M^{p}; of which the corresponding monovalent moiety L^{p} comprises a functional group W^{p} that is capable of forming a covalent bond with the binding protein; M^{p}, when present, is a Stretcher unit; L^{M} is a bond, or a trivalent or tetravalent linker, and when L^{M} is a bond, a₂ is 1, when L^{M} is trivalent linker, a₂ is 2, or when L^{M} is a tetravalent linker, a₃ is 3; L³, when present, is a carbonyl-containing moiety; M^{A} comprises a peptide moiety that contains at least two amino acids; T¹ is a hydrophilic group and the [symbol] between T¹ and M^{A} denotes direct or indirect attachment of T¹ and M^{A}; each occurrence of D is independently a GR agonist payload, wherein one H has been replaced with a covalent bond to L^{D}; and each occurrence of L^{D} is independently a divalent linker moiety connecting D to M^{A} and comprises at least one cleavable bond such that when the bond is broken, D is released in an active form for its intended therapeutic effect (e.g., as a GR agonist).

In certain embodiments, the instant disclosure relates to a binding protein suitable for forming a conjugate of a compound of Categories A to K comprising a Category III linker, represented by Formulae (LIII-3) or (LIII-4):

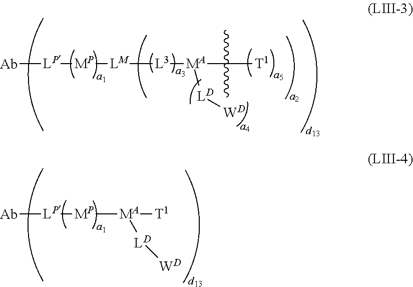

wherein:
a₁ when present, is an integer from 0 to 1; a₂, when present, is an integer from 1 to 3; a₃, when present, is an integer from 0 to 1; a₄, when present, is an integer from 1 to about 5; as when present, is an integer from 1 to 3; d₁₃ is an integer from 1 to about 6; Ab represents a binding protein of this disclosure; L^{p′} is a divalent linker moiety connecting the antibody to M^{p}; of which the corresponding monovalent moiety L^{p} comprises a functional group W^{p} that is capable of forming a covalent bond with a functional group of the antibody; M^{p}, when present, is a Stretcher unit; L^{M} when present, is a bond, or a trivalent or tetravalent linker, and when L^{M} is a bond, a 2 is 1, when L^{M} is a trivalent linker, a₂ is 2, or when L^{M} is a tetravalent linker, a₂ is 3; L³, when present, is a carbonyl-containing moiety; M^{A} comprises a peptide moiety that contains at least two amino acids; T¹ is a hydrophilic group; and the [symbol] between T¹ and M^{A} denotes direct or indirect attachment of T¹ and M^{A}; each occurrence of W^{D} when present, is independently a functional group that is capable of forming a covalent bond with a functional group of a compound of Structure I or II; and each occurrence of L^{o} is independently a divalent linker moiety connecting W^{D} or D to M^{A} and L^{o} comprises at least one cleavable bond such that when the bond is broken, D is released in an active form for its intended therapeutic effect.

In one embodiment, the peptide moiety in Category III linkers comprises from three to about ten amino acids, e.g., about 3, about 4, about 5, about 6, about 7, about 8, about 9, or about 10 amino acids.

In one embodiment, the hydrophilic group comprises:

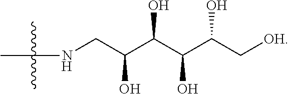

In another embodiment, the hydrophilic group comprises:

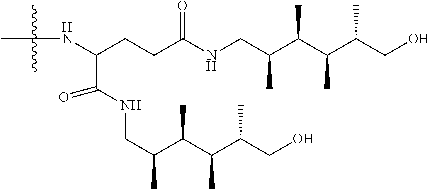

In some embodiments, the amino polyalcohol is

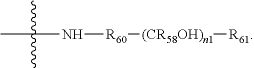

wherein
m is an integer from 0 to about 6; each R₅₈, when present, is independently hydrogen or C₁₋₈ alkyl; R₆₀ is a bond, a C₁₋₆ alkyl linker, or —CHR₅₉— in which R₅₉ is —H, C₁₋₈ alkyl, cycloalkyl, or arylalkyl; R₆₁ is CH₂OR₆₂, COOR₆₂, —(CH₂)n2COOR₆₂, or a heterocycloalkyl substituted with one or more hydroxyl; R₆₂ is H or C₁₋₈ alkyl; and n₂ is an integer from 1 to about 5.

In some embodiments, the hydrophilic group comprises:

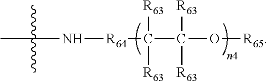

wherein:
n₄ is an integer from 1 to about 25; each R₆₃ is independently hydrogen or C₁₋₈ alkyl; R₆₄ is a bond or a C₁₋₈ alkyl linker; R₆₂ is H, C₁₋₈ alkyl, or —(CH₂)ₙ₂COOR₆₂; R₆₂ is H or C₁₋₈ alkyl; and n₂ is an integer from 1 to about 5.

In certain embodiments, the hydrophilic group comprises polyethylene glycol, e.g., polyethylene glycol with from about 6 to about 24 PEG subunits. In some embodiments, the hydrophilic group comprises a polyethylene glycol with from about 6 to about 12 PEG subunits.

In some embodiments, the hydrophilic group comprises a polyethylene glycol with from about 8 to about 12 PEG subunits.

In further embodiments, L³, when present, comprises —X—C₁₋₁₀ alkylene-C(Q)-, with X directly connected to L^{M}, in which X is CH₂, O, or NR₅, and R₅ is hydrogen, C₁₋₆ alkyl, C₆₋₁₀ aryl, C₃₋₈ cycloalkyl, COOH, or COO—C₁₋₆ alkyl.

In some embodiments, L³, when present, is —NR5-(CH₂)v-C(O)— or —CH₂—(CH₂)v-C(O)—NR5-(CH2)v-C(O)—, in which each v independently is an integer from 1 to 10 (e.g., each v independently being an integer from 1 to 6, or from 2 to 4, or 2). In some embodiments, LIII is —NH—(CH₂)₂—C(O)— or H(CH₂)₂—C(O)—NH—(CH₂)₂—C(O)—.

In some embodiments, a₄ is 1, 2, or 3. In some embodiments, d₁₃ is an integer from about 1 to about 6. In some embodiments, d₁₃ is an integer from about 1 to about 4. In some embodiments, d₁₃ is an integer from about 4 to about 6. In some embodiments, d₁₃ is an integer from about 2 to about 4. In some embodiments, d₁₃ is an integer from about 1 to about 2. In some embodiments, d₁₃ is 2. In some embodiments, each W^{p}, when present, is independently:

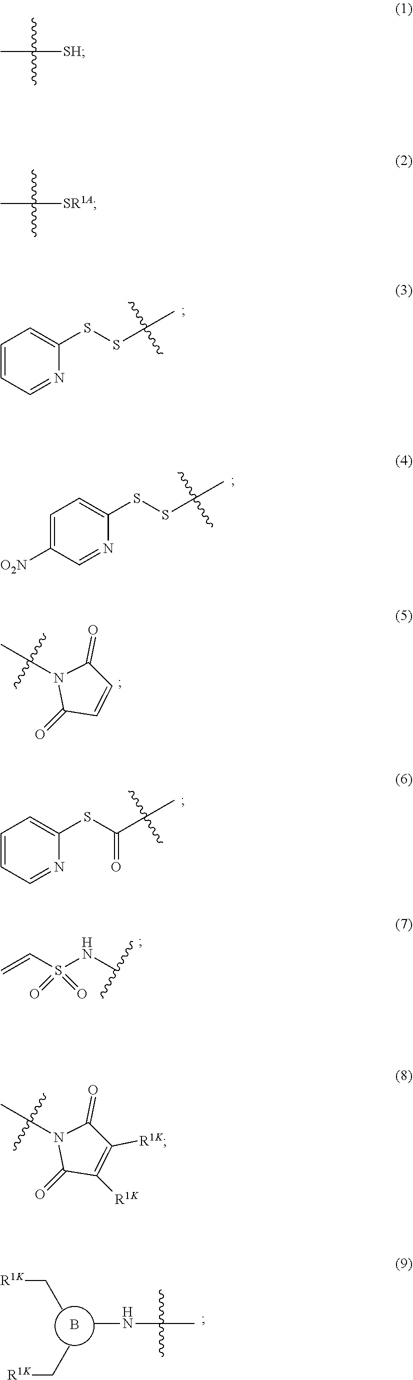

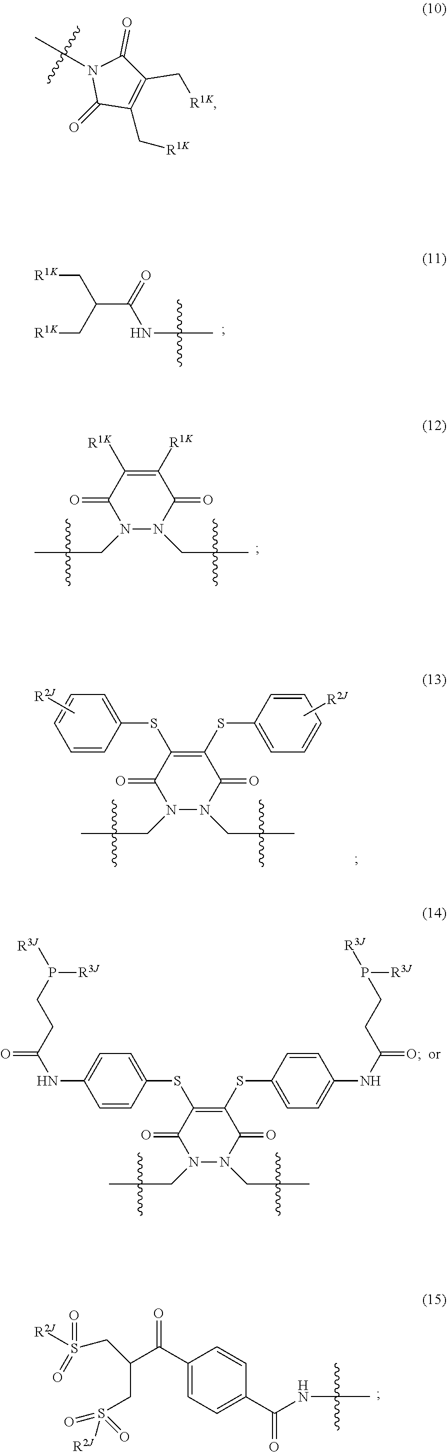

wherein
ring B is cycloalkyl, heterocycloalkyl, aryl, or heteroaryl;
R^{1K}— is a leaving group;
R^{1A} is a sulfur protecting group;
R^{2J} is hydrogen, an aliphatic, aryl, heteroaliphatic, or carbocyclic moiety; and
R^{3J} is C₁₋₆ alkyl and each of Z₁, Z₂, Z₃, and Z₇ is independently a carbon or nitrogen atom.

In some embodiments, R^{1K} is halo or RC(O)O— in which R is hydrogen, an aliphatic, heteroaliphatic, carbocyclic, or heterocycloalkyl moiety.

In one embodiment,
R^{1A} is

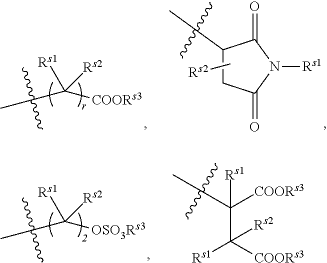

wherein r is 1 or 2 and each of R^{s1}, R^{s2}, and R^{s3} heteroaliphatic moiety, a carbocyclic moiety, or a heterocycloalkyl moiety.

In some embodiments, each W^{P} is independently

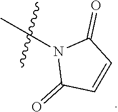 [SMILES: *N1C(=O)C=CC1=O]

R^{s2}, and R^{s3} is hydrogen, an aliphatic, heteroaliphatic, carbocyclic, or heterocycloalkyl moiety.

In some embodiments, W^{P} is

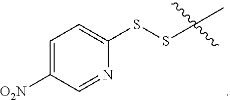 [SMILES: *SSc1ccc([N+](=O)[O-])cn1]

In some embodiments, when
W^{P} is

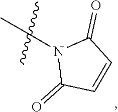 [SMILES: *N1C(=O)C=CC1=O]

L^{P′} comprises

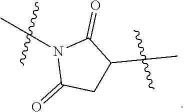 [SMILES: *C1CC(=O)N(*)C1=O]

In some embodiments,
W_{P} is

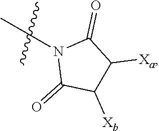

wherein one of Xa and Xb is H and the other is a maleimido blocking moiety. In some embodiments, a maleimido blocking compound (i.e., a compound that can react with maleimide to convert it to succinimide) may be used to quench the reaction between, e.g., the Linker-Drug moiety and the PERM (e.g., the engineered cysteine of the PERM), and a maleimido blocking moiety refers to the chemical moiety attached to the succinimide upon conversion. In some embodiments, the maleimido blocking moieties are moieties that can be covalently attached to one of the two olefin carbon atoms upon reaction of the maleimido group with a thiol-containing compound of Formula (LIII-7)

R90-(CH₂)_{d}—SH (LIII-7)
wherein:
R₉₀ is NHR₉₁, OH, COOR₉₃, CH(NHR₉₁)COOR₉₃, or a substituted phenyl group;
R₉₃ is hydrogen or C1-4 alkyl;
R₉₁ is hydrogen, CH₃, or CH₂CO and
d is an integer from 1 to 3.

In some embodiments, the maleimido blocking compound can be cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is optionally substituted with one or more hydrophilic substituents, or 1-aminopropane-1-thiol. In some embodiments, the one or more hydrophilic substituents on phenyl comprise OH, SH, methoxy, ethoxy, COOH, CHO, COC₁₋₄ alkyl, F, cyano, SO₃H, PO₃H, and the like.

In some embodiments, the maleimido blocking group is —S—(CH₂)_{d}—R₉₀, in which, R₉₀ is OH, COOH, or CH(NHR₉₁)COOR₉₃;
R₉₃ is hydrogen or CH₃;
R₉₁ is hydrogen or CH₃CO; and
d is 1 or 2.

In one embodiments, the maleimido blocking group is —S—CH₂—CH(NH₂)COOH.

In one embodiment, the stretcher unit, M^{p} is a group of its own.

In some embodiments, M^{p}, when present, is —(Z₄)—[(Z₅)—(Z₆)]z with Z₄ connected to L^{p′} or L^{p} and Z₆ connected to L^{M}; wherein z is 1, 2, or 3;

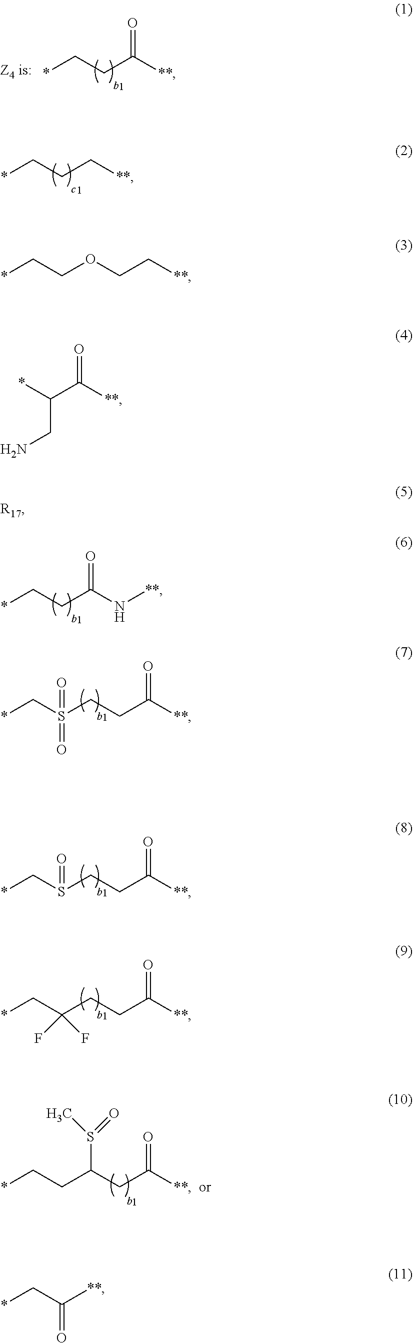

wherein * denotes attachment to L^{p′} or L^{p} and ** denotes attachment to Z₅ or Z₆, when present, or to L^{M} when Z₅ and Z₆ are both absent;
b₁ is an integer from 0 to 6;
e₁ is an integer from 0 to 8,
R₁₇ is C₁₋₁₀ alkylene, C₁₋₁₀ heteroalkylene, C₃₋₈ cycloalkylene, O—(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4- to 14-membered heterocycloalkylene, —C₁₋₁₀ alkylene-(4- to 14-membered heterocycloalkylene)-, -(4- to 14-membered heterocycloalkylene)-C₁₋₁₀ alkylene-, —C₁₋₁₀ alkylene-C(═O)—, —C₁₋₁₀ heteroalkylene-C(═O)—, -C3-8 cycloalkylene-C(═O)—, —O-(C1-8 alkyl)-C(═O)—, -arylene-C(═O)—, -C1-10 alkylene-arylene-C(═O)—, -arylene-C1-10 alkylene-C(═O)—, -C1-10 alkylene-(C3-s cycloalkylene)-C(═O)—, -(C3-8 cycloalkylene)-C₁₋₁₀alkylene-C(═O)—, 4 to 14-membered heterocyeloalkylene-C(═O)—, -C1-10 alkylene-(4- to 14-membered heterocycloalkylene)-C(═O))—, -(4- to 14-membered heterocycloalkylene)-C₁₋₁₀ alkylene-C(═O)—, -C1-10 alkylene-NH—, -C1-10 heteroalkylene-NH—, -C3-8 cycloalkylene-NH—, —O-(C1-8 alkyl)-NH—, -arylene-NH—, -C1-10 alkylene-arylene-NH—, -arylene-C1-10 alkylene-NH—, -C1-10 alkylene-(C3-8 cycloalkylene)-NH—, -(C3-8 cycloalkylene)-C1-10 alkylene-NH—, -4- to 14-membered heterocycloalkylene-NH—, -C1-10 alkylene-(4 to 14-membered heterocycloalkylene)-NH—, -(4 to 14-membered heterocycloalkylene)-C1-10 alkylene-NH—, -C1-10 alkylene-S—, -C1-10 heteroalkylene-S—, -C3-8 cycloalkylene-S—, —O-C1-8 alkyl)-S—, -arylene-S—, -C1-10 alkylene-arylene-S—, -arylene-C1-10 alkylene-S—, -C1-10 alkylene-(C3-8 cycloalkylene)-S—, -(C3-8 cycloalkylene)-C1-10 alkylene-S—, -4 to 14-membered heterocycloalkylene-S—, -C1-10 alkylene-(4 to 14-membered heterocycloalkylene)-S—, or -(4 to 14-membered heterocycloalkyl ene)-C1-10 alkylene-S—;
each Z₅ independently is absent, R57-R17 or a polyether unit;
each R₅₇ independently is a bond, NR23, S or 0;
each R₂₃ independently is hydrogen, C1-6 alkyl, C6-10 aryl, C3-8 cycloalkyl, COOH, or COO-C1-6 alkyl; and
each Z6 independently is absent, -C1-10 alkyl-R5-, -C1-10 alkyl-NR5-, -C1-10 alkyl-C(O)—, -C1-10 alkyl-O—, -C1-10 alkyl-S— or -(C1-1) alkyl-R3)_{g1}-C1-10 alkyl-C(O)—;
each R₃ independently is —C(O)—NR5- or —NR5-C(O)—;
each R5 independently is hydrogen, C1-6 alkyl, Ce-io aryl, C3-8 cycloalkyl, COOH, or COO—C1-6 alkyl; and
g₁ is an integer from 1 to 4.

In some embodiments, Z₄ is

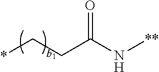

In other embodiments, Z₄ is

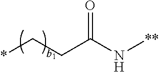

wherein b₁ is 1 or 4.

In some embodiments, Z₄ is

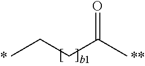

In other embodiments, Z₄ is

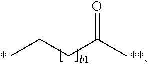

e.g. wherein bi is 1

In other embodiments, Z₄ is

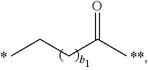

e.g., wherein bi is 0.

In some embodiments, Z₄ is

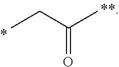

In other embodiments,
Z₄ is

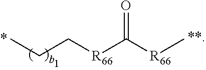

In some embodiments, b₁ is 0. In some embodiments, one of R₆₆ is O, and the other is NH.

In some embodiments, Z₄ is

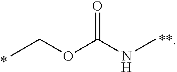

In some embodiments,

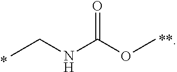

In some embodiments, each Z₅ independently is a polyalkylene glycol (PAO), including but are not limited to, polymers of lower alkylene oxides (e.g., polymers of ethylene oxide, such as, for example, propylene oxide, polypropylene glycols, polyethylene glycol (PEG), polyoxyethylenated polyols, copolymers thereof and block copolymers thereof). In some embodiments, the polyalkylene glycol is a polyethylene glycol (PEG) including polydisperse PEG, monodisperse PEG and discrete PEG. in some embodiments, polydisperse PEGS are a heterogeneous mixture of sizes and molecular weights whereas monodisperse PEGS are purified from heterogeneous mixtures and therefore provide a single chain length and molecular weight. In some embodiments, the PEG units are discrete PEGs. In some embodiments, the discrete PEGs provide a single molecule with defined and specified chain length. In some embodiments, the PEG is mPEG.

As used herein a subunit when referring to the PEG unit refers to a polyethylene glycol subunit having the formula

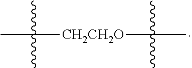

In some such embodiments, the PEG unit comprises multiple PEG subunits.

In some embodiments, when z is 2 or 3, at least one Z₅ is a polyalkylene glycol (PAO), e.g., a PEG unit.

In some embodiments, when z is 2, at least one Z₅ is a polyalkylene glycol (PAO), e.g., a PEG unit.

In some embodiments, when z is 3, at least one Z₅ is a polyalkylene glycol (PAO), e.g., a PEG unit.

In some embodiments, the PEG unit comprises 1 to 6 subunits. In some embodiments, the PEG unit comprises 1 to 4 subunits. In some embodiments, the PEG unit comprises 1 to 3 subunits. In some embodiments, the PEG unit comprises 1 subunit. In some embodiments, the PEG unit comprises 2 subunits. In some embodiments, the PEG unit comprises 3 subunits. In some embodiments, the PEG unit comprises 4 subunits. In some embodiments, the PEG unit comprises 5 subunits. In some embodiments, the PEG unit comprises 6 subunits.

In some embodiments, the PEG unit comprises one or multiple PEG subunits linked together by a PEG linking unit. In some embodiments, the PEG linking unit that connects one or more chains of repeating CH₂CH₂O— subunits is Z₆. In some embodiments, Z₆ is —C1-10 alkyl-R3-, —C₂₋₁₀ alkyl-NH—, —C2-10 alkyl-C(O)—, -C2-10 alkyl-O— or -C1-10 alkyl-S, wherein R3 is —C( ))—CR5- or —NR5-C(O)—.

In some embodiments, the PEG linking unit is —C1-10 alkyl-C(O)—NH— or —C1-10 alkyl-NH—C(O)—. In some embodiments, the PEG linking unit is —C1-10 alkyl-C(O)—NH—. In some embodiments, the PEG linking unit is —C1-10 alkyl-NH—C(O)—.

In some embodiments, the PEG linking unit is —(CH₂)₂—C(O)—NH—.

In some embodiments, each Z₅ is absent.

In some embodiments, when z is 2 or 3, at least one Z₅ is absent.

In some embodiments, when z is 2, at least one Z₅ is absent. In some embodiments, when z is 3, at least one Z₅ is absent.

In some embodiments, each Z₅ is —(CH₂—CH₂—O-)₂-.

In some embodiments, when z is 2 or 3, at least one Z₅ is —(CH₂—CH2-O—)₂—. In some embodiments, when z is 2, at least one Z5 is —(CH₂-CH2-O—)₂—. In some embodiments, when z is 3, at least one Z5 is —(CH₂—CH₂—O-)₂—

In some embodiments, each Z5 independently is R₅₇—R₁₇. In some embodiments, each Z₅ independently is R₁₇, NHR₁₇, OR₁₇, or SR₁₇.

In some embodiments, when z is 2 or 3, at least one Z₅ is R₅₇—R₁₇ (e.g., R17, NHR17, OR17, or SR:—)

In some embodiments, when z is 2, at least one Z₅ is R₅₇—R₁₇ (e.g., R17, NHR17, OR17, or SR17). In some embodiments, when z is 3, at least one Z₅ is R57-R17 (e.g., R17, NHR17, OR17, or SR17).

In some embodiments, each Z₆ is absent.

In some embodiments, when z is 2 or 3, at least one Z₆ is absent.

In some embodiments, when z is 2, at least one Z₆ is absent. In some embodiments, when z is 3, at least one Z₆ is absent.

In some embodiments, at least one of Z₅ and Z₆ is not absent.

In some embodiments, each Z₆ independently is —C₁₋₁₀ alkyl-R₃—, —C₁₋₁₀ alkyl-NH—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—.

In some embodiments, g₁ is an integer from 1 to 4.

In some embodiments, when z is 2 or 3, at least one Z₆ is —C₁₋₁₀ alkyl-R₃-, —C₁₋₁₀ alkyl-NH—, —C₁₋₁₀ alkyl-C(O)—, —C₁₋₁₀ alkyl-O—, —C₁₋₁₀ alkyl-S—, or —(C₁₋₁₀ alkyl-R₃)_{g1}—C₁₋₁₀ alkyl-C(O)—. In some embodiments, g₁ is an integer from 1 to 4.

In some embodiments, each Z₆ independently is —C₂₋₁₀ alkyl-C(O)— (e.g., —(—(CH₂)₂C(O)—).

In some embodiments, at least one Z₆ is —C₂₋₁₀ alkyl-C(O)— (e.g., —(—(CH₂)₂C(O)—).

In some embodiments, each Z₆ independently is —C₂₋₁₀ alkyl-R₃-C₂₋₁₀ alkyl-C(O)— (e.g., —(CH₂)₂—C(O)_{w}-(CH₂)₂—C(O)—).

In some embodiments, at least one Z₆ is —C₂₋₁₀ alkyl-R₃—C₂₋₁₀ alkyl-C(O)— (e.g., —(CH₂)₂—C(O)NH—(CH₂)₂—C(O)—).

In some embodiments, each Z₆ independently is —(C₂₋₁₀ alkyl-R₃)_{g1}—C₂₋₁₀ alkyl-C(O)— (e.g., —(CH₂)₂—C(O)NH—(CH₂)₂—NHC(O)—(CH₂)—C(O)—).

In some embodiments, at least one Z₆ is —(C₂₋₁₀ alkyl-R₃)_{g1}—C₂₋₁₀ alkyl-C(O)— (e.g., —(CH₂)₂—C(O)NH—(CH₂)₂—NHC(O)—(CH₂)—C(O)—) or —(CH₂)₂—NH—C(O)—(CH₂)₂—C(O)— NH—(CH₂)—C(O)—).

In some embodiments, each Z₆ independently is —(CH₂)₂—NH—C(O)—(CH₂)₂—C(O)— NH—(CH₂)—C(O)—).

In some embodiments, —[(Z₅)—(Z₆)]z- is not absent.

In some embodiments, —[(Z₅)—(Z₆)]z- is a bond.

In some embodiments, —[(Z₅)—(Z₆)]z- is —(CH₂CH₂O)₂—(CH₂)₂—C(O)—

In some embodiments, —[(Z₅)—(Z₆)]z- is —(CH₂CH₂O)₂—(CH₂)₂—C(O)—NH—(CH₂CH₂O)₂—.

In some embodiments, —[(Z₅)—(Z₆)]z- is —(CH₂CH₂O)₂—(CH₂)₂—C(O)—NH—(CH₂)—C(O).

In some embodiments, M^{p}, when present, is

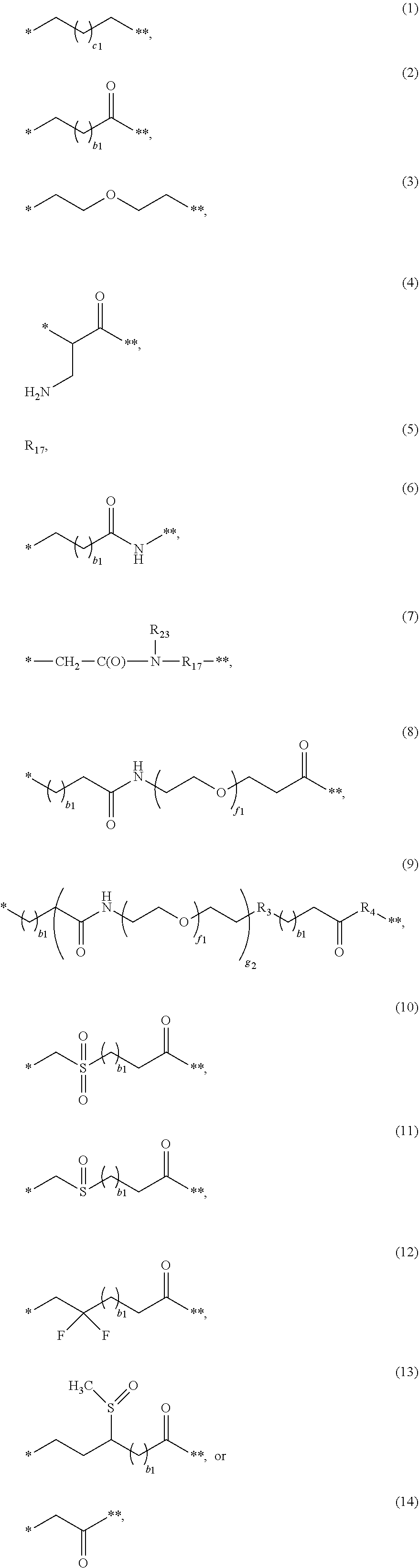

wherein * denotes attachment to L′ or L′ and ** denotes attachment to I^{M};
R₃ is —C(O)—N5 or —NR₅—C(O)—;
R₄ is a bond or —NR₅—(CR₂₀R₂₁)—C(O)—;
R₅ is hydrogen, C1-6 alkyl, C6-10aryl, C₃₋₈ cycloalkyl, —COOH, or —COO—C₁₋₆ alkyl;
R₁₇ is C₁₋₁₀ alkylene, C₁₋₁₀ heteroalkylene, C₃₋₈ cycloalkylene, O-(C₁₋₈ alkylene, arylene, —C₁₋₁₀ alkylene-arylene-, -arylene-C₁₋₁₀ alkylene-, —C₁₋₁₀ alkylene-(C₃₋₈ cycloalkylene)-, —(C₃₋₈ cycloalkylene-C₁₋₁₀ alkylene-, 4- to 14-membered heterocycloalkylene, —C₁₋₁₀ alkylene-(4- to 14-membered heterocycloalkylene)-, -(4- to 14-membered heterocycloalkylene)-C₁₋₁₀ alkylene-, —C₁₋₁₀ alkylene-C(═O)—, —C₁₋₁₀ heteroalkylene-C(═O)—, —C₃₋₈ cycloalkylene-C(═O)—, —O-(C₁₋₈ alkyl)-C(═O)—, -arylene-C(═O)—, —C₁₋₁₀ alkylene-arylene-C(═O)—, -arylene-C₁₋₁₀ alkylene-C(═O)—, —C₁₋₁₀ alkylene-(C₃₋₈ cycloalkylene)-C(═O)—, —(C₃₋₈ cycloalkylene)-C₁₋₁₀alkylene-C(═O)—, 4 to 14-membered heterocyeloalkylene-C(═O)—, —C₁₋₁₀ alkylene-(4- to 14-membered heterocycloalkylene)-C(═O))—, -(4- to 14-membered heterocycloalkylene)-C₁₋₁₀ alkylene-C(═O)—, —C₁₋₁₀ alkylene-NH—, —C₁₋₁₀ heteroalkylene-NH—, —C₃₋₈ cycloalkylene-NH—, —O-(C₁₋₈ alkyl)-NH—, -arylene-NH—, —C₁₋₁₀ alkylene-arylene-NH—, -arylene-C₁₋₁₀ alkylene-NH—, —C₁₋₁₀ alkylene-(C₃₋₈ cycloalkylene)-NH—, —(C₃₋₈ cycloalkylene)-C₁₋₁₀ alkylene-NH—, -4- to 14-membered heterocycloalkylene-NH—, —C₁₋₁₀ alkylene-(4 to 14-membered heterocycloalkylene)-NH—, -(4 to 14-membered heterocycloalkylene)-C₁₋₁₀ alkylene-NH—, —C₁₋₁₀ alkylene-S—, —C₁₋₁₀ heteroalkylene-S—, —C₃₋₈ cycloalkylene-S—, —O—C₁₋₈ alkyl)-S—, -arylene-S—, —C₁₋₁₀ alkylene-arylene-S—, -arylene-C₁₋₁₀ alkylene-S—, —C₁₋₁₀ alkylene-(C₃₋₈ cycloalkylene)-S—, —(C₃₋₈ cycloalkylene)-C₁₋₁₀ alkylene-S—, -4 to 14-membered heterocycloalkylene-S—, —C₁₋₁₀ alkylene-(4 to 14-membered heterocycloalkylene)-S—, or -(4 to 14-membered heterocycloalkylene)-C₁₋₁₀ alkylene-S—; each Rao and Rai independently is hydrogen, C₁₋₆ alkyl, C₆₋₁₀ aryl, hydroxylated C₆₋₁₀ aryl, polyhydroxylated C₆₋₁₀ aryl, 5- to 12-membered heterocycle, C₃₋₈ cycloalkyl, hydroxylated C₃₋₈ cycloalkyl, polyhydroxylated C₃₋₈ cycloalkyl or a side chain of a natural or unnatural amino acid: each R₂₃ independently is hydrogen, C₁₋₆ alkyl, C₆₋₁₀ aryl, C3-8 cycloalkyl, COOH, or COO—C₁₋₆ alkyl;
each b₁ independently is an integer from 0 to 6;
e₁ is an integer from 0 to 8;
each f₁ independently is an integer from 1 to 6; and
g₂ is an integer from 1 to 4.

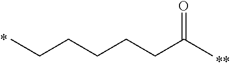

In some embodiments, M^{p}, when present, is

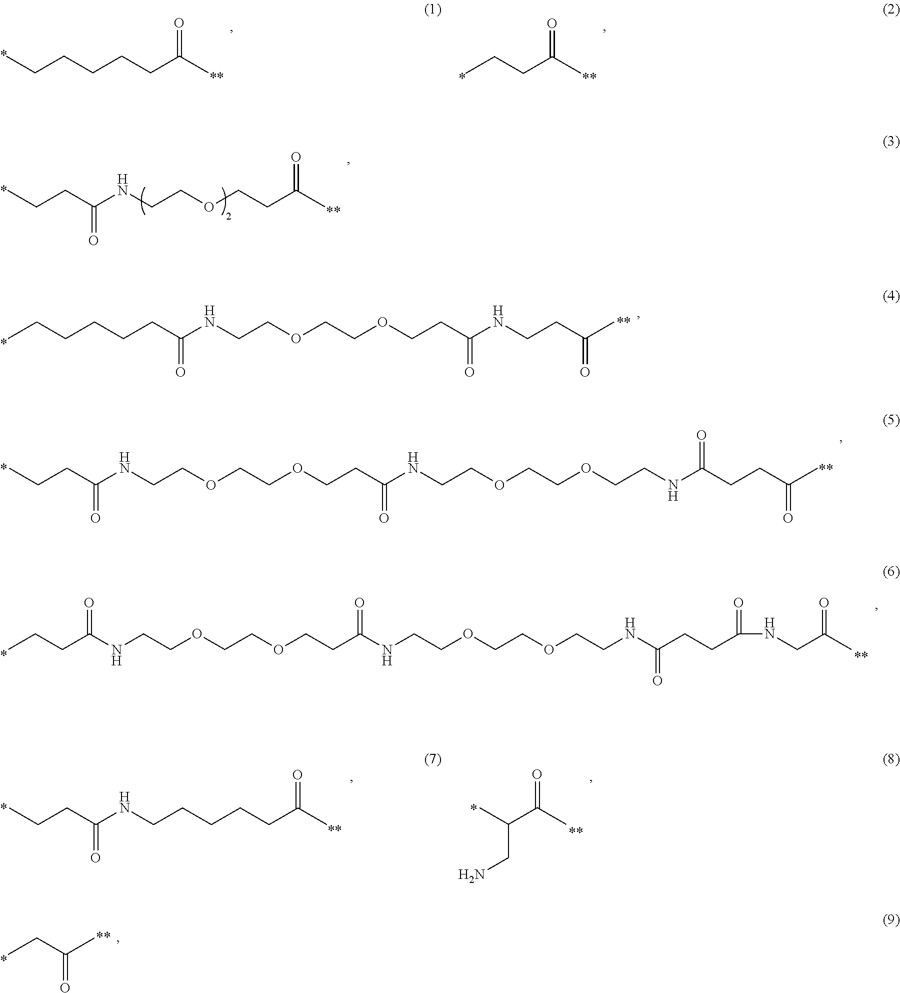

wherein * denotes attachment to L^{p′} or L^{p} and ** denotes attachment to L^{M}

In one embodiment, L^{M} is a bond and a₂ is 1.

In some embodiments, a₂ is 2, and L^{M} is

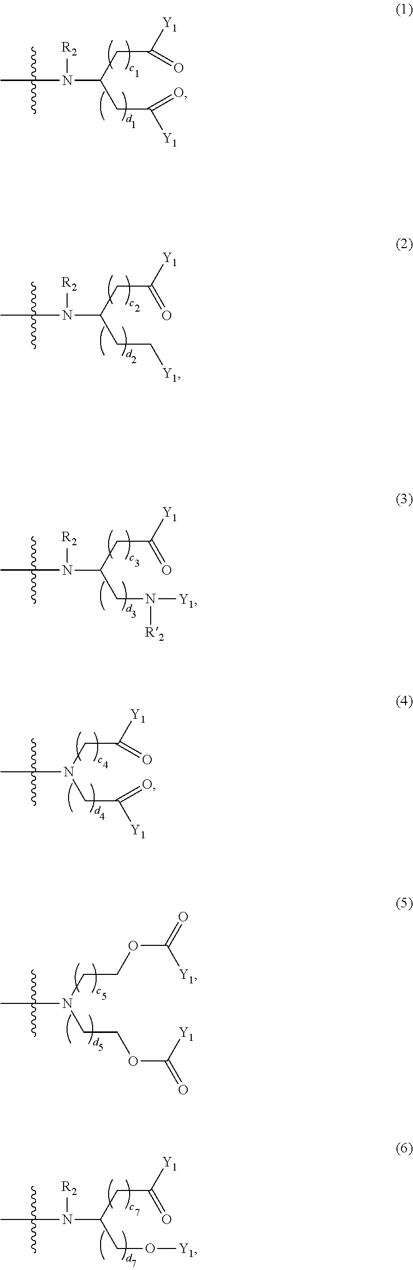

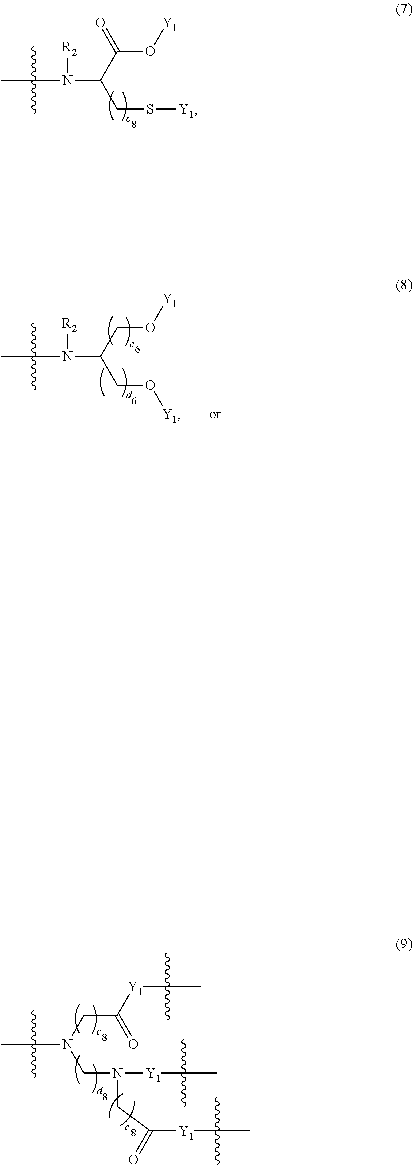

wherein
[symbol] denotes attachment to M^{p} when present or attachment to L^{p} or L^{p′} when M^{p} is absent;
Y₁ denotes attachment to L^{III} when present or attachment to M^{A} when L^{III} is absent;
R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxy carbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, COOH, or COO—C₁₋₆ alkyl;
each of c₁, c₂, c₃, c₄, c₅, c₇, and c₈ is an integer independently ranging between 0 and 10; and
each of d₁, d₂, d₃, d₄, d₅, and d₇ is an integer independently ranging between 0 and 10.

In some embodiments,
a₂ is 2 and L^{M} is:

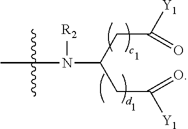

In some embodiments, a₂ is 3 and L^{M} is:

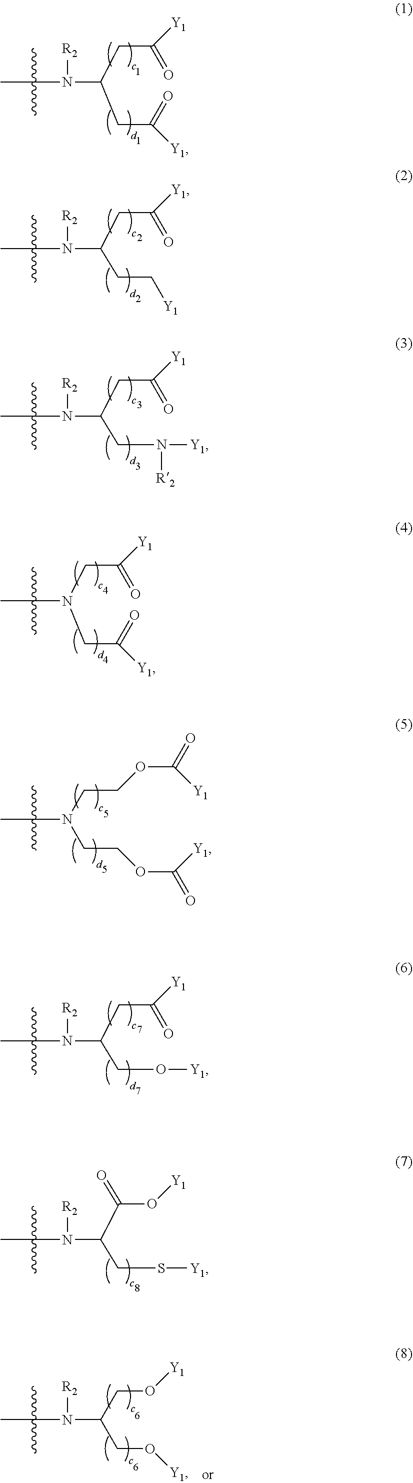

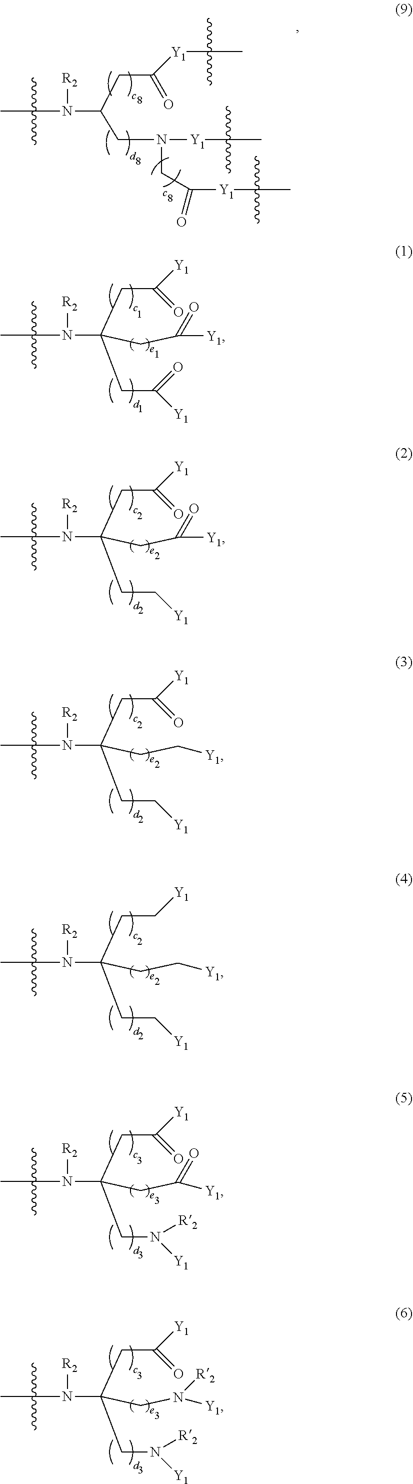

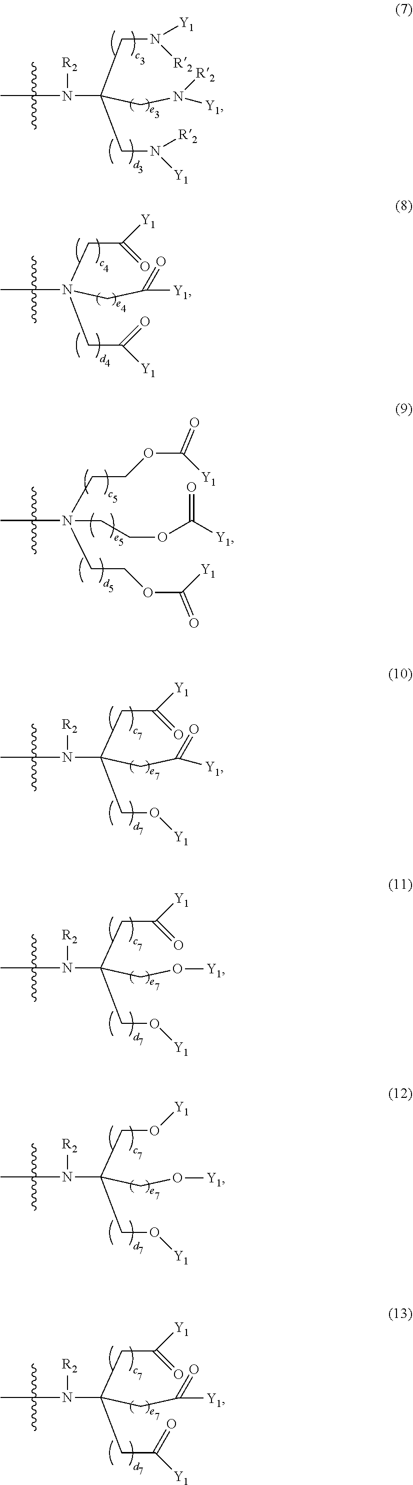

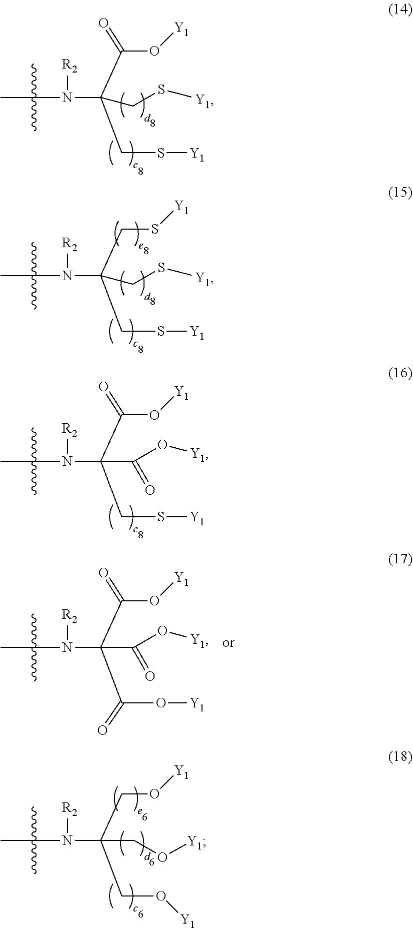

wherein: [symbol] denotes attachment to M^{p} when present or attachment to L^{p} or L^{p′} wherein M^{p} is absent; Y₁ denotes attachment to L³ when present or attachment to M^{A} when L³ is absent; R₂ and R′₂ are each independently hydrogen, an optionally substituted C₁₋₆ alkyl, an optionally substituted C₂₋₆ alkenyl, an optionally substituted C₂₋₆ alkynyl, an optionally substituted C₃₋₁₉ branched alkyl, an optionally substituted C₃₋₈ cycloalkyl, an optionally substituted C₆₋₁₀ aryl, an optionally substituted heteroaryl, an optionally substituted C₁₋₆ heteroalkyl, C₁₋₆ alkoxy, aryloxy, C₁₋₆ heteroalkoxy, C₂₋₆ alkanoyl, an optionally substituted arylcarbonyl, C₂₋₆ alkoxy carbonyl, C₂₋₆ alkanoyloxy, arylcarbonyloxy, an optionally substituted C₂₋₆ alkanoyl, an optionally substituted C₂₋₆ alkanoyloxy, an optionally substituted C₂₋₆ substituted alkanoyloxy, COOH, or COO—C₁₋₆ alkyl;
each of c₁, c₂, c₃, c₄, c₅, c6, c₇, and c₈ is an integer independently ranging between 0 and 10;
each of d₁, d₂, d₃, d₄, d₅, d₆, d₇ and d₈ is an integer independently ranging between 0 and 10; and
each of e₁, e₂, e₃, e₄, e₅, e₆, e₇ and e₈ is an integer independently ranging between 0 and 10.

In some embodiments,
a₂ is 3 and L^{M} is

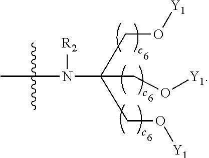

In one embodiment, the peptide moiety, M^{A} is a group of its own.

In other embodiments, M^{A} comprises a peptide moiety that comprises at least about five amino acids.

In one embodiment, M^{A} comprises a peptide moiety that comprises at most about sixteen amino acids.

In some embodiments, M^{A} comprises a peptide moiety that comprises about 4, about 5, about 6, about 7, about 8, about 9, about 10, about 11, about 12, about 13, about 14, about 15, or about 16 amino acids.

In some embodiments, M^{A} comprises a peptide moiety that comprises at most about ten amino acids.

In some embodiments, M^{A} comprises a peptide moiety that comprises about 4, about 5, about 6, about 7, about 8, about 9, or about 10 amino acids.

In some embodiments, M^{A} comprises a peptide moiety that comprises from about three to about ten amino acids selected from glycine, serine, glutamic acid, aspartic acid, lysine, cysteine, a stereoisomer thereof (e.g., isoglutamic acid or isoaspartic acid), and a combination thereof.

In some embodiments, M^{A} comprises a peptide moiety that comprises at least four glycines and at least one serine.

In some embodiments, M^{A} comprises a peptide moiety that comprises at least four glycines and at least one glutamic acid.

In some embodiments, M^{A} comprises a peptide moiety that comprises at least four glycines, at least one serine and at least one glutamic acid.

In some embodiments, the peptide moiety comprises:

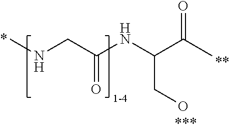

wherein:
* indicates attachment to L³ when present, or to L^{M} when L³ is absent; ** indicates attachment to T¹ when present, or —OH when T¹ is absent; and *** indicates attachment to L^{D} when present, or —H when L^{D} is absent.

In some embodiments, the peptide moiety comprises (glycine)-(serine), wherein:
the peptide moiety is attached to L³ when present, or to L^{M} when L³ is absent, via the glycine; the peptide moiety is attached to T¹ when present, via the serine; and the peptide moiety is attached to L^{D} when present, via the serine.

In some embodiments, the peptide moiety comprises

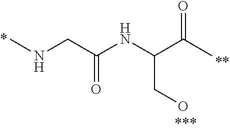

wherein:
* indicates attachment to L³ when present, or to L^{M} when L³ is absent;
** indicates attachment to T¹ when present, or —OH when T¹ is absent; and
*** indicates attachment to L^{D} when present, or —H when L^{o} is absent.

In some embodiments, the peptide moiety comprises (glycine)₄-(serine), wherein: the peptide moiety is attached to L³ when present, or to L^{M} when L³ is absent, via one of the glycine; the peptide moiety is attached to T¹ when present, via the serine; and the peptide moiety is attached to L^{D} when present, via the serine.

In some embodiments, the peptide moiety comprises:

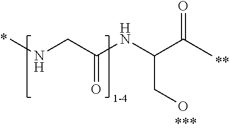

wherein:
* indicates attachment to L³ when present, or to L^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises (serine)-(glycine)₄, wherein: the peptide moiety is attached to L³ when present, or to L^{M} when L³ is absent, via the serine; the peptide moiety is attached to T¹ when present, via one of the glycine; and the peptide moiety is attached to L^{D} when present, via the serine.

In some embodiments, the peptide moiety comprises

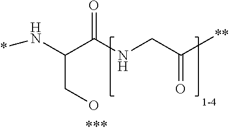

wherein
* indicates attachment to L³ when present, or to L^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises:

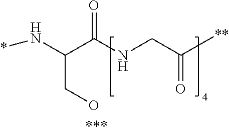

wherein:
* indicates attachment to L³ when present, or to I.^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises (-alanine)-(glycine)₁₋₄-(serine), wherein:
the peptide moiety is attached to L³ when present, or to L^{M} when L³ is absent, via the β-alanine;
the peptide moiety is attached to T¹ when present, via the serine; and
the peptide moiety is attached to L^{D} when present, via the serine.

In some embodiments, the peptide moiety comprises:

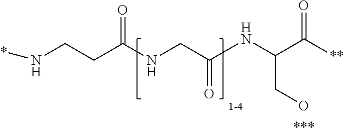

wherein:
* indicates attachment to L³ when present, or to L^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises (P-alanine)-(glycine)₄-(serine), wherein:
the peptide moiety is attached to L³ when present, or to L^{M} when L³ is absent, via the β-alanine;
the peptide moiety is attached to T¹ when present, via the serine; and
the peptide moiety is attached to L^{D} when present, via the serine.

In some embodiments, the peptide moiety comprises:

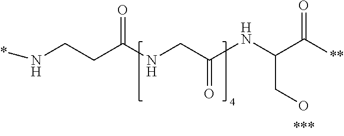

wherein:
* indicates attachment to L³ when present, or to L^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises (glycine)₁₋₄-(glutamic acid), wherein:
the peptide moiety is attached to L³ when present, or to L^{M} when L³ is absent, via one of the glycine;
the peptide moiety is attached to T¹ when present, via the glutamic acid; and
the peptide moiety is attached to L^{D} when present, via the glutamic acid.

In some embodiments, the peptide moiety comprises (glycine)₁₋₄-(glutamic acid, wherein: the peptide moiety is attached to L³ when present, or to L^{M} when L³ is absent, via the glutamic acid;
the peptide moiety is attached to T¹ when present, via the glycine; and
the peptide moiety is attached to L^{D} when present, via the glutamic acid.

In some embodiments, the peptide moiety comprises:

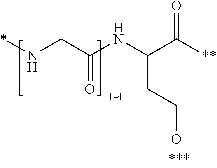

wherein:
* indicates attachment to L³ when present, or to L^{M} when L³ is absent;
** indicates attachment to T¹ when present, or —OH when T¹ is absent; and
*** indicates attachment to L^{D} when present, or —H when L^{D} is absent.

In some embodiments, the peptide moiety comprises (glycine)-(glutamic acid), wherein:
the peptide moiety is attached to L when present, or to L^{M} when L³ is absent, via the glycine;
the peptide moiety is attached to T¹ when present, via the glutamic acid; and the peptide moiety is attached to L^{D} when present, via the glutamic acid.

In some embodiments, the peptide moiety comprises:

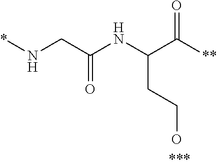

wherein:
* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments, the peptide moiety comprises (glycine)₄-(glutamic acid), wherein: the peptide moiety is attached to L³ when present, or to L^{M} when L³ is absent, via one of the glycine;
the peptide moiety is attached to T¹ when present, via the glutamic acid; and
the peptide moiety is attached to L^{D} when present, via the glutamic acid.

In some embodiments, the peptide moiety comprises:

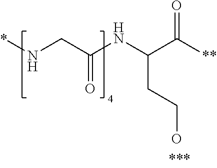

wherein:
* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments, the peptide moiety comprises (glutamic acid)-(glycine)₄, wherein:
the peptide moiety is attached to L³ when present, or to L^{M} when L³ is absent, via the glutamic acid;
the peptide moiety is attached to T¹ when present, via one of the glycine; and
the peptide moiety is attached to L^{D} when present, via the glutamic acid.

In some embodiments, the peptide moiety comprises:

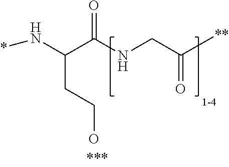

* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments, the peptide moiety comprises:

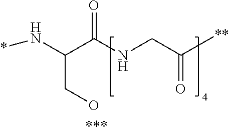

wherein:
* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments, the peptide moiety comprises (p-alanine)-(glycine)₁₋₄-(glutamic acid), wherein:
the peptide moiety is attached to L³ when present, or to L^{M} when L³ is absent, via the β-alanine;
the peptide moiety is attached to T¹ when present, via the glutamic acid; and the peptide moiety is attached to L^{D} when present, via the glutamic acid

In some embodiments, the peptide moiety comprises:

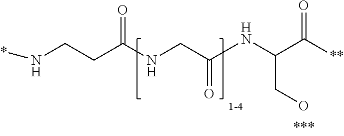

wherein:
* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments, the peptide moiety comprises (β-alanine)-(glycine)₄-(glutamic acid), wherein:
the peptide moiety is attached to L³m when present, or to L^{M} when L³ is absent, via the β-alanine;
the peptide moiety is attached to T¹ when present, via the glutamic acid; and the peptide moiety s attached to L^{D} when present, via the glutamic acid.

In some embodiments, the peptide moiety comprises:

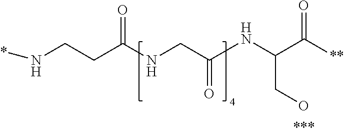

wherein:
* indicates attachment to L³ when present, or to L^{M} when L³ is absent;
** indicates attachment to T¹ when present, or —OH when T¹ is absent; and
*** indicates attachment to LD when present, or —H when L^{D} is absent.

In some embodiments of linker L^{III}, each occurrence of L^{D} is independently a divalent linker moiety connecting D to M^{A} and comprises at least one cleavable bond such that when the bond is cleaved, D is released in an active form for its intended therapeutic effect.

In some embodiments, L^{D} is a component of the Releasable Assembly Unit. In other embodiments, L^{D} is the Releasable Assembly Unit.

In some embodiments, L^{D} comprises one cleavable bond.

In some embodiments, L^{D} comprises multiple cleavage sites or bonds.

In some embodiments, functional groups for forming a cleavable bond can include, for example, sulfhydryl groups to form disulfide bonds, aldehyde, ketone, or hydrazine groups to form hydrazone bonds, hydroxylamine groups to form oxime bonds, carboxylic or amino groups to form peptide bonds, carboxylic or hydroxy groups to form ester bonds, and sugars to form glycosidic bonds. In some embodiments, L^{D} comprises a disulfide bond that is cleavable through disulfide exchange, an acid-labile bond that is cleavable at acidic pH, or bonds that are cleavable by hydrolases (e.g., peptidases, esterases, and glucuronidases).

In some embodiments, L^{D} comprises a carbamate bond (i.e., —O—C(O)—NR—, in which R is H or alkyl or the like).

In some embodiments, the structure and sequence of the cleavable bond in I^{D} can be such that the bond is cleaved by the action of enzymes present at the target site. In other embodiments, the cleavable bond can be cleavable by other mechanisms.

In some embodiments, the structure and sequence of the cleavable bonds in L^{D} can be such that the bonds are cleaved by the action of enzymes present at the target site. In other embodiments, the cleavable bonds can be cleavable by other mechanisms.

In some embodiments, the cleavable bond(s) can be enzymatically cleaved by one or more enzymes, including a tumor-associated protease, to liberate the linker-payload, wherein the conjugate of the present disclosure, or intermediate, or scaffold thereof, is protonated in vivo upon release to provide a linker-payload.

In some embodiments, L^{D} can comprise one or more amino acids. In some embodiments, for example, each amino acid in L^{D} can be natural or unnatural or a D or L isomer, provided that there is a cleavable bond. In some embodiments, L^{D} comprises an alpha, beta, or gamma amino acid that can be natural or non-natural. In some embodiments, L^{D} comprises 1 to 12 (e.g., 1 to 6, or 1 to 4, or 1 to 3, or 1, 2, 3, 4, 5, 6, 7, 8, 9, 10, 11, or 12) amino acids in contiguous sequence.

In some embodiments, L^{D} can comprise only natural amino acids. In some embodiments, L^{o} can comprise only non-natural amino acids. In some embodiments, L^{D} can comprise a natural amino acid linked to a non-natural amino acid. In some embodiments, L^{D} can comprise a natural amino acid linked to a D-isomer of a natural amino acid. In some embodiments, L^{D} comprises a dipeptide such as -Val-Cit-, -Phe-Lys-, or -Val-Ala-.

In some embodiments, L^{D} comprises a monopeptide, a dipeptide, a tripeptide, a tetrapeptide, a pentapeptide, a hexapeptide, a heptapeptide, an octapeptide, a nonapeptide, a decapeptide, an undecapeptide, or a dodecapeptide unit.

In some embodiments, L^{D} comprises a peptide (e.g., of 1 to 12 amino acids), which is conjugated directly to the payload. In some such embodiments, the peptide is a single amino acid or a dipeptide. In some such embodiments, the peptide is a single amino acid. In some such embodiments, the peptide is a dipeptide.

In some embodiments, each amino acid in L^{D} is independently selected from alanine, βalanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoaikanedioic acid, aminobenzoic acid, amino-heterocyclo-alkanoic acid, heterocyclo-carboxylic acid, citrulline, statine, diaminoaikanoic acid, and derivatives thereof.

In some embodiments, each amino acid is independently selected from alanine, b alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, citrulline, and selenocysteine.

In some embodiments, each amino acid is independently selected from the group consisting of alanine, b-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline, and derivatives thereof.

In some embodiments, each amino acid is selected from the proteinogenic or the non proteinogenic amino acids.

In some embodiments, each amino acid in L^{D} can be independently selected from L or D isomers of the following amino acids: alanine, beta-alanine, arginine, aspartic acid, asparagine, cysteine, histidine, glycine, glutamic acid, glutamine, phenylalanine, lysine, leucine, methionine, serine, tyrosine, threonine, tryptophan, proline, ornithine, penicillamine, aminoalkynoic acid, aminoalkanedioic acid, heterocyclo-carboxylic acid, statine, diaminoalkanoic acid, valine, citrulline, and derivatives thereof.

In some embodiments, each amino acid in L^{D} is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, citrulline, or alanine.

In some embodiments, each amino acid in L^{D} is independently selected from L-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in L^{D} is independently selected from D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in L^{D} independently is L- or D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, or valine.

In some embodiments, each amino acid in L^{D} is alanine, β-alanine, glutamic acid, isoglutamic acid, isoaspartic acid, valine, citrulline, or aspartic acid.

In some embodiments, L^{o} comprises β-alanine. In some embodiments, L^{D} comprises (β-alanine -alanine). In some embodiments, L^{D} comprises β-alanine-(glutamic acid). In some embodiments, L^{D} comprises (β-alanine)-(isoglutamic acid). In some embodiments, L^{D} comprises (β-alanine)-(aspartic acid). In some embodiments, L^{D} comprises (β-alanine)-(isoaspartic acid). In some embodiments, L^{D} comprises (β-alanine)-(valine). In some embodiments, L^{D} comprises (β-alanine)-(valine)-(alanine). In some embodiments, L^{D} comprises (β-alanine)-(alanine)-(alanine).

In some embodiments, L^{D} comprises (β-alanine)-(valine)-(citrulline).

In some embodiments, L^{D} comprises a carbamate bond in addition to one or more amino acids.

In some embodiments, L^{D} can be designed and optimized in selectivity for enzymatic cleavage by a particular enzyme. In some embodiments, the enzyme is a tumor-associated protease.

In some embodiments, L^{D} comprises a bond whose cleavage is catalyzed by cathepsin B, C and D, or a plasmin protease.

In some embodiments, L^{D} comprises a sugar cleavage site. In some embodiments, L^{D} comprises a sugar moiety (Su) linked via an oxygen glycosidic bond to a self-immolative group. In some embodiments, a “self-immolative group” can be a tri-functional chemical moiety that is capable of covalently linking together three spaced chemical moieties (i.e., the sugar moiety (via a glycosidic bond), a GR agonist payload (directly or indirectly), and M^{A} (directly or indirectly). In some embodiments, the glycosidic bond can be cleaved at the target site to initiate a self-immolative reaction sequence that leads to a release of the drug.

In some embodiments, L^{D} comprises a sugar moiety (Su) linked via a glycoside bond (0′-) to a self-immolative group (K) of the formula:

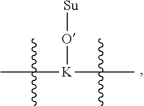

wherein the self-immolative group (K) forms a covalent bond with the GR agonist payload (directly or indirectly) and forms a covalent bond with M^{A} (directly or indirectly). In some embodiments, examples of self-immolative groups are described in WO 2015/057699, the contents of which are hereby incorporated by reference in its entirety.

In some embodiments, L^{D}, when not connected to or prior to connecting to a drug comprises a functional group W^{D}. In some embodiments, each W^{D} independently can be a functional group as listed for W^{p}. In some embodiments, each W^{D} independently is

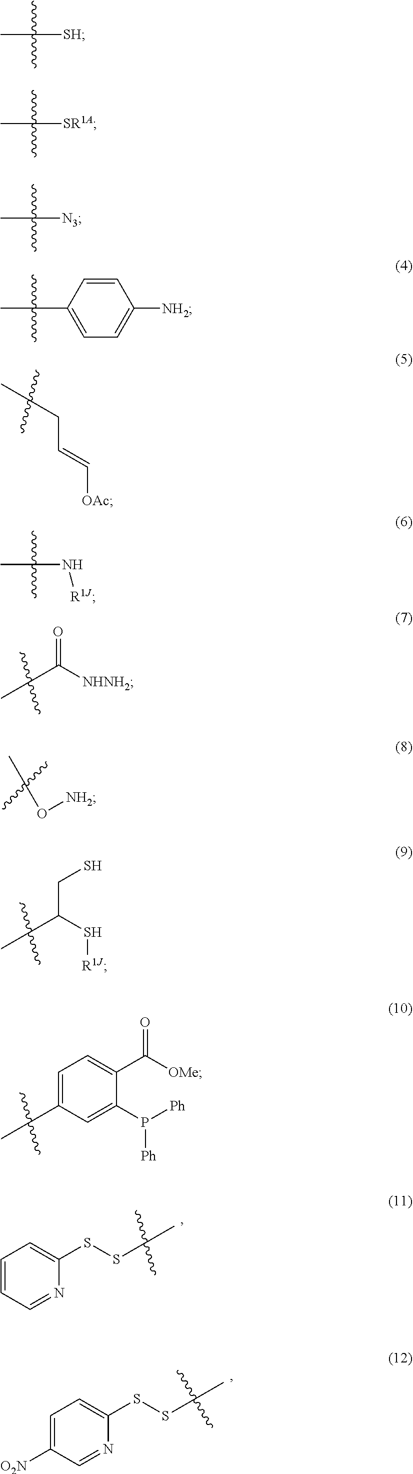

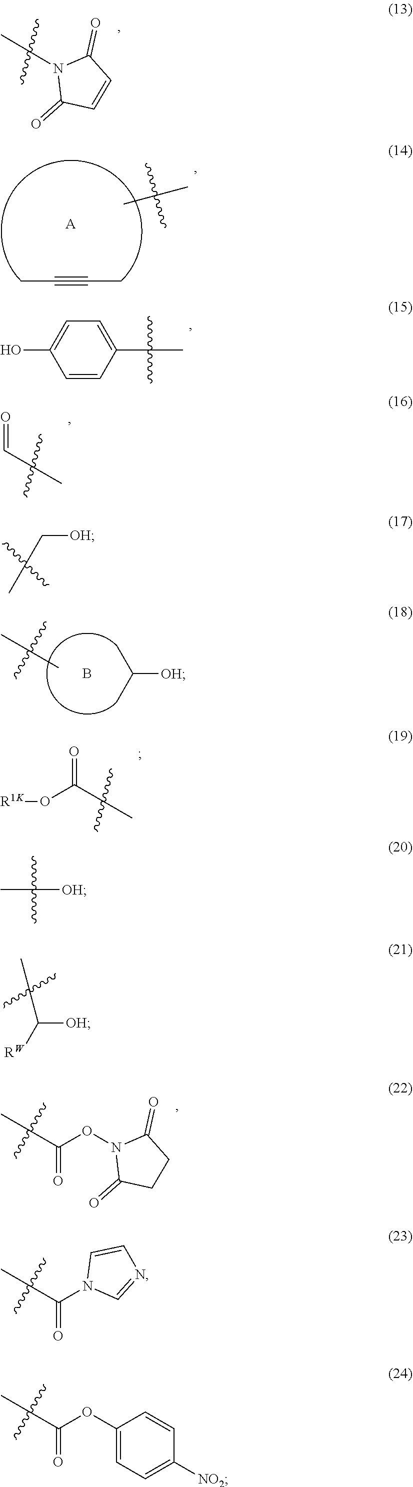

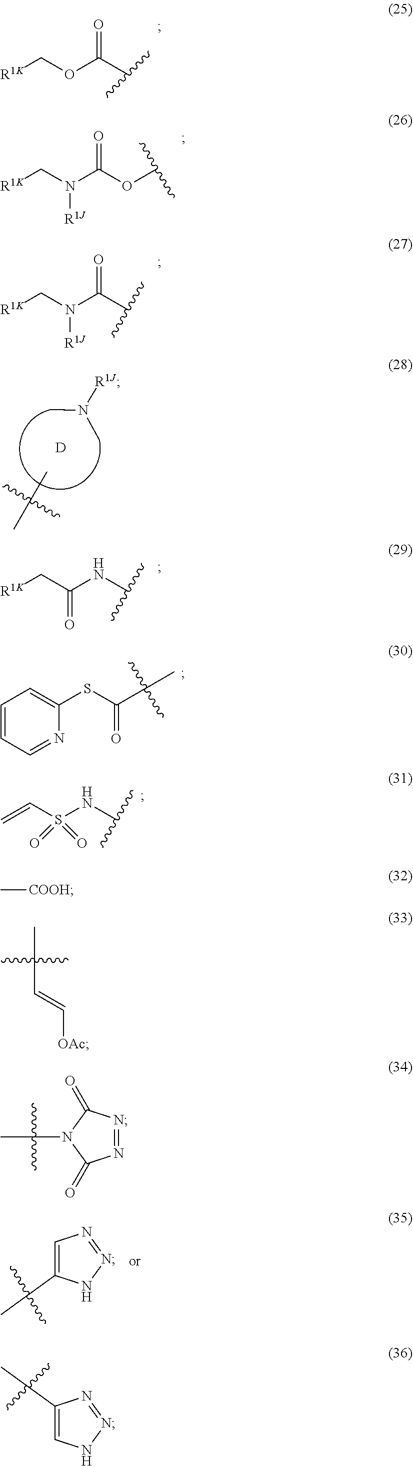

in which R^{1A} is a sulfur protecting group, each of ring A and B, independently, is cycloalkyl or heterocycloalkyl; R^{W} is an aliphatic, heteroaliphatic, carbocyclic, or heterocycloalkyl moiety; ring D is heterocycloalkyl; R^{1J} is hydrogen, an aliphatic, heteroaliphatic, carbocyclic, or heterocycloalkyl moiety; and R^{1K} is a leaving group (e.g., halide or RC(O)O— in which R is hydrogen, an aliphatic, heteroaliphatic, carbocyclic, or heterocycloalkyl moiety).

4. Category IV Linkers

In different embodiments, such as embodiments wherein the GR agonist includes a hydroxyl moiety, the drug-linker includes a Category IV linker represented by formula (LIV-1):

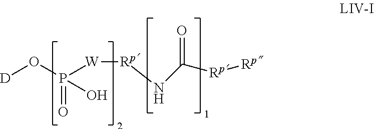

or a pharmaceutically acceptable salt thereof, wherein: D-O is a portion of a hydroxyl-containing compound of Categories A to K, wherein the hydroxyl-substituted compound has the formula D-OH; W is 0 or N; R^{p} is C₁-C₆ alkylene or phenyl; y is 0 or 1;
R^{p′} is absent, —CH₂O, —(CH₂CH₂O)ₙ; wherein n is 1-10, or —C₁-C₆ alkylene; and R^{p″} is a reactive group capable of forming a covalent bond with reactive side chains of a binding protein of this disclosure.

In some embodiments, R^{p′} is —(CH₂CH₂O)ₙ; wherein n is 1-10, or —C₁-C₆ alkylene.

In other embodiments, Rp″ is:

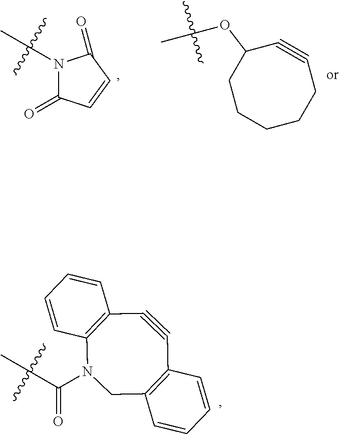

wherein [symbol] is the point of attachment to the rest of drug-linker.

In one embodiment, the Category IC linker is represented by formula (LIV-2) or (LIV-3):

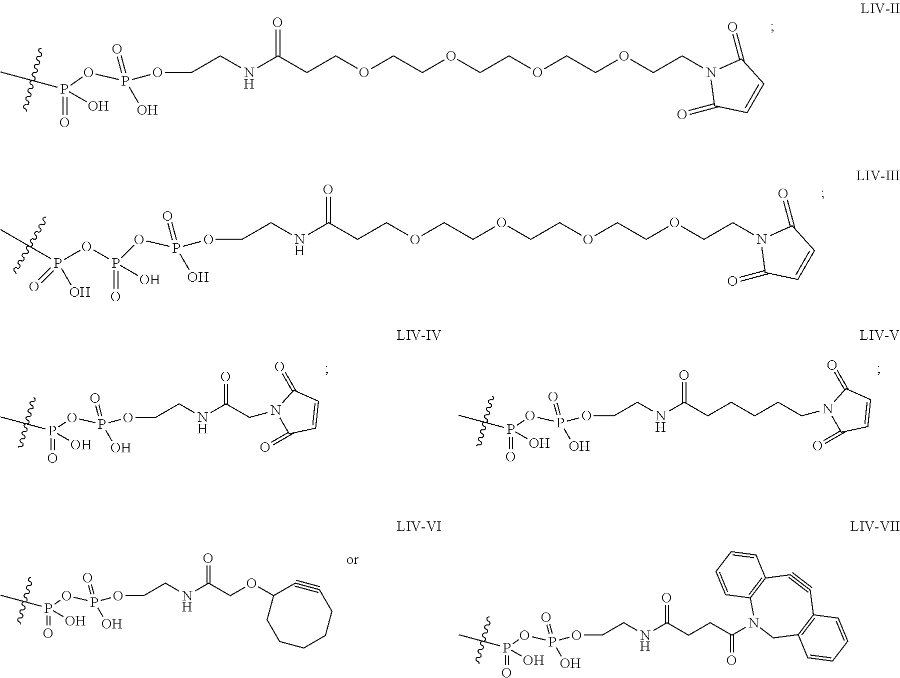

wherein [symbol] is the point of attachment to an oxygen atom in a compound of Categories A to K.

In various other embodiments, conjugates comprising a Category IV linker, have one of the following structures (LIV-VIII) or (LIV-IX):

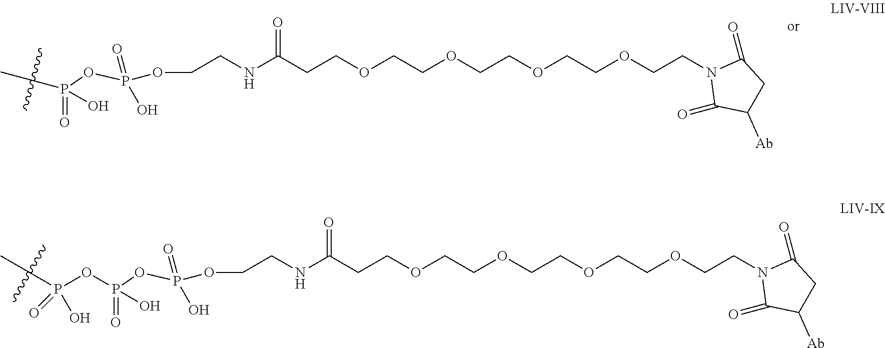

In one embodiment, polypeptide is a multifunctional antibody and can be used to link one or more Linker-payload moieties. In certain embodiments, the ratio between Linker-payload moiety and the antibody is about 6:1, about 5:1, about 4:1, about 3:1, about 2:1, or about 1:1. In some embodiments, the ratio between the compound of Categories A to K and the antibody is about 6:1, 5:1, 4:1, 3:1, about 2:1, or about 1:1.

5. Category V Linkers

Exemplary linkers are described in some detail in, for example, PCT Publication No. WO 2019/106608, the linkers of which is incorporated by reference herein in their entirety. In some specific embodiments, the linker has the following structure:

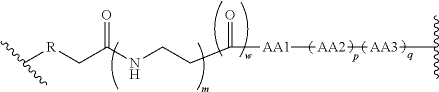

wherein R, AA1, AA2, AA3, m, w, p, and q are as defined in PCT Publication No. WO 2019/106608.

In certain embodiments, the linker has one of the following structures:

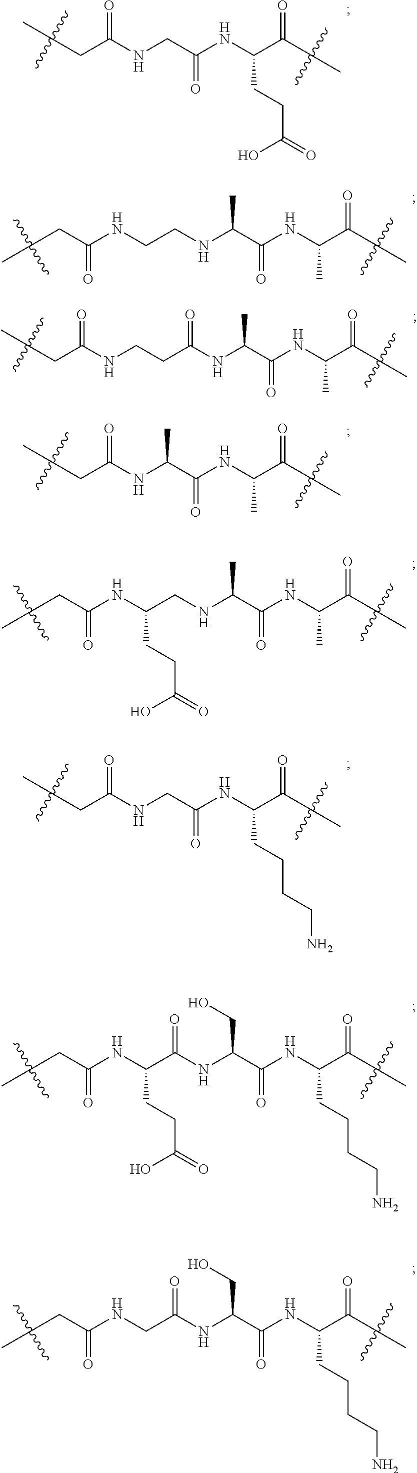

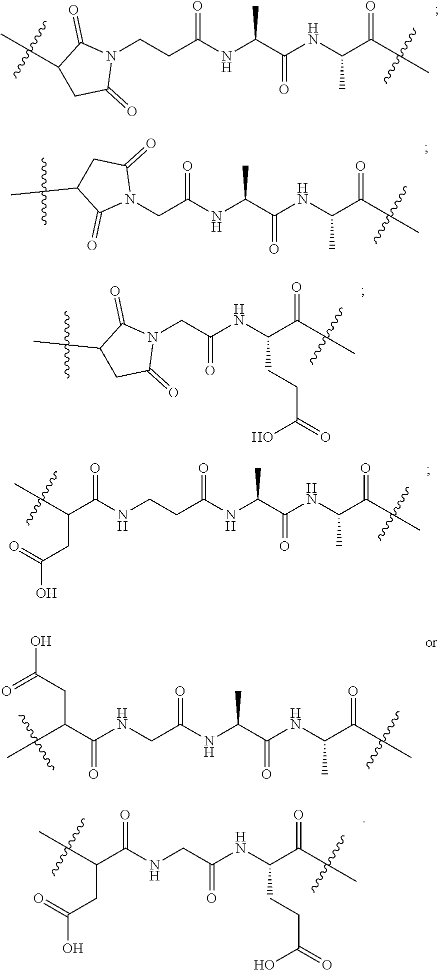

6. Category VI Linkers

Exemplary linkers are described in some detail in, for example: PCT Publication No. WO 2017/210471, US Publication No. US 2019/0167804, and U.S. Pat. Nos. 10,772,970 and 10,668,167, the contents of each of which are incorporated by reference herein in their entirety. In some specific embodiments, the linker is -L-Q- wherein L is a linker and Q is a heterobifunctional group, a heterotrifunctional group, or absent. In some more specific embodiments, Q is a heterobifunctional group selected from the group consisting of:

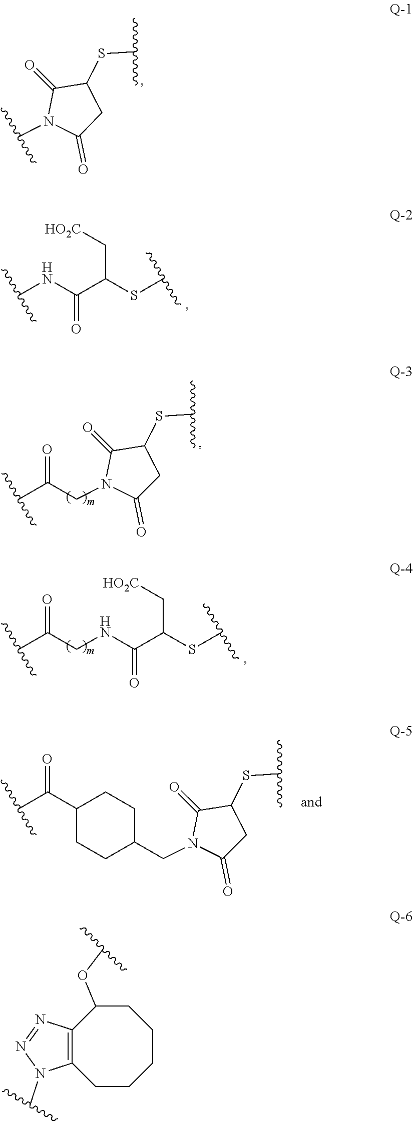

wherein m is 1, 2, 3, 4, 5, or 6.

In some embodiments, Q is a heterotrifuncitonal group that is

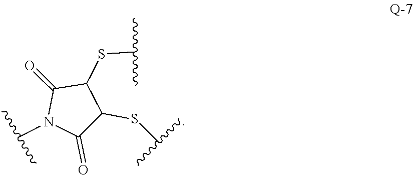 [SMILES: *SC1C(=O)N(*)C(=O)C1S*]

In some more specific embodiments, -L-Q- is:

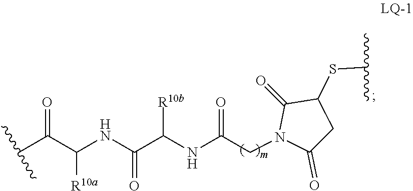

wherein m is 2 or 3; and
R^{10a} and R^{10b} are independently selected from the group consisting of hydrogen and optionally substituted C₁₋₆ alkyl. In another embodiment, m is 2. In another embodiment, m is 1. In another embodiment, -L-Q- is:

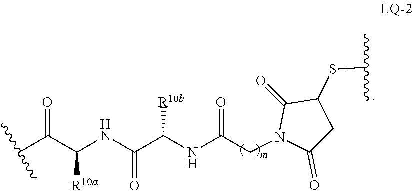

In another embodiment, -L-Q- is:

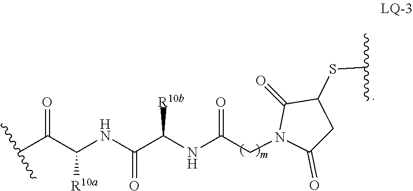

In another embodiment, -L-Q- is:

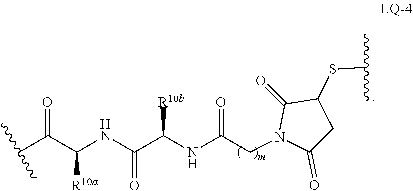

In another embodiment, -L-Q- is:

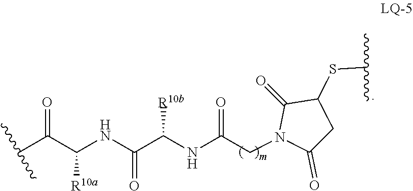

In some embodiments, -L-Q- is:

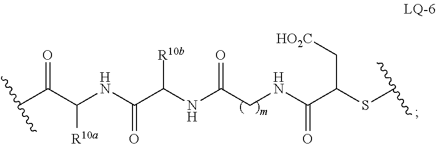

wherein m is 2 or 3; and R^{10a} and R^{10b} are independently selected from the group consisting of hydrogen and optionally substituted C₁₋₆ alkyl. In another embodiment, m is 2. In another embodiment, -L-Q- is:

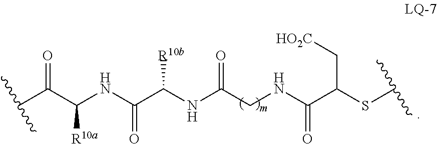

In another embodiment, -L-Q- is:

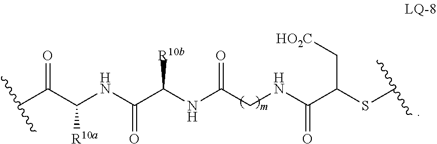

In another embodiment, -L-Q- is:

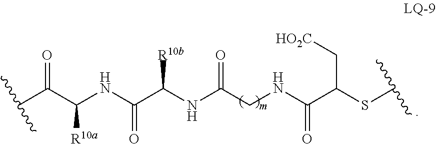

In another embodiment, -L-Q- is:

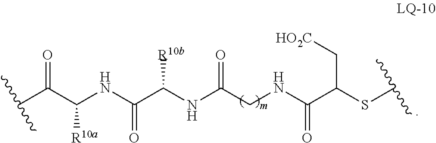

In some embodiments, L is a non-cleavable linker. In another embodiment, the linker (L) comprises one or more polyethylene glycol units.

In some embodiments, -L-Q- is:

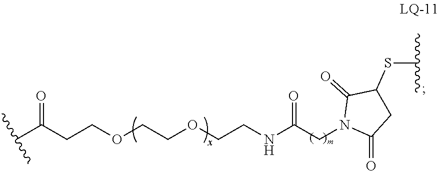

m is 2 or 3; and x is 0, 1, 2, 3, 4, 5, 6, 7, 8, 9, 10, 11, 12, 13, 14, or 15. In another embodiment, m is 2.

In some embodiments, -L-Q- is:

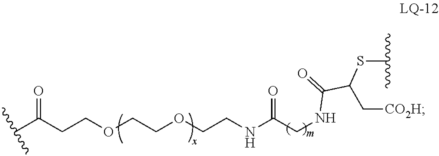

m is 2 or 3; and x is 0, 1, 2, 3, 4, 5, 6, 7, 8, 9, 10, 11, 12, 13, 14, or 15. In another embodiment, m is 2.

In some embodiments, -L-Q- is:

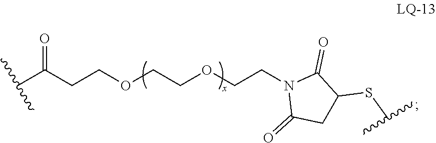

and x is 0, 1, 2, 3, 4, 5, 6, 7, 8, 9, 10, 11, 12, 13, 14, or 15.

In some embodiments, -L-Q- is:

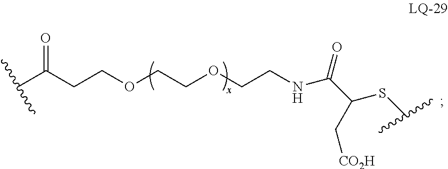

and x is 0, 1, 2, 3, 4, 5, 6, 7, 8, 9, 10, 11, 12, 13, 14, or 15.

In some embodiments, -L-Q- is:

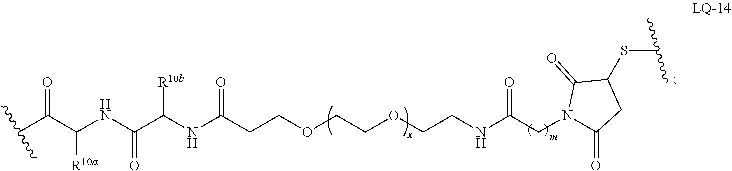

m is 1 or 2; x is 0, 1, 2, 3, 4, 5, 6, 7, 8, 9, 10, 11, 12, 13, 14, or 15; and R^{10a} and R^{10b} are independently selected from the group consisting of hydrogen and optionally substituted C₁₋₆ alkyl.

In another embodiment, -L-Q- is:

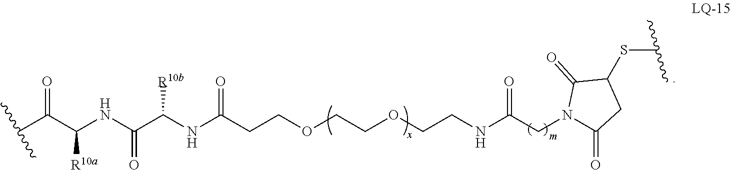

In another embodiment, -L-Q- is:

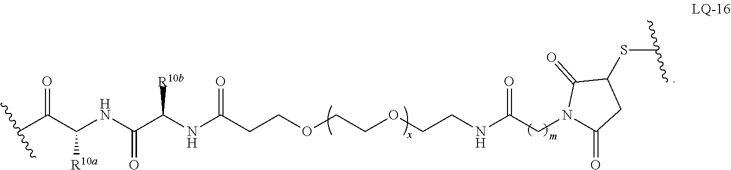

In another embodiment, -L-Q- is:

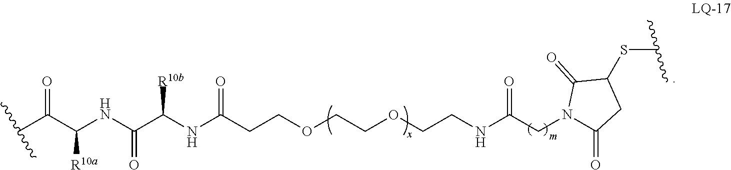

In another embodiment, -L-Q- is:

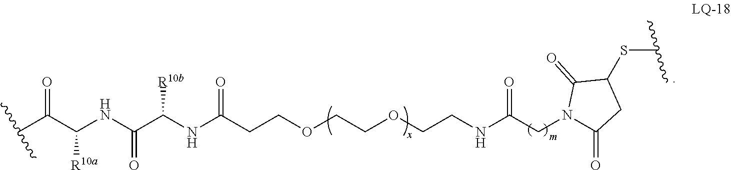

In some embodiments, -L-Q- is:

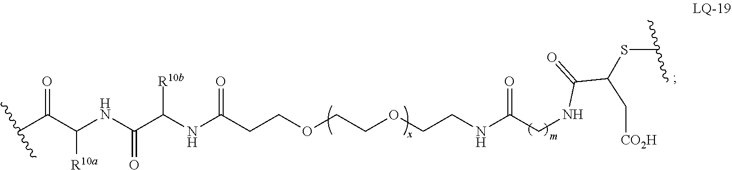

m is 1 or 2; x is 0, 1, 2, 3, 4, 5, 6, 7, 8, 9, 10, 11, 12, 13, 14, or 15; and R^{10a} and R^{10b} are independently selected from the group consisting of hydrogen and optionally substituted C₁₋₆ alkyl.

In another embodiment, -L-Q- is:

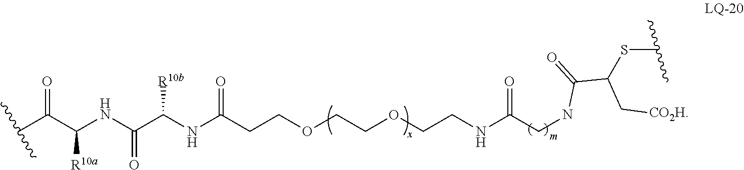

In another embodiment, -L-Q- is:

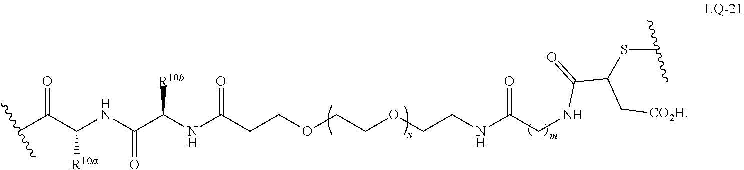

In another embodiment, -L-Q- is:

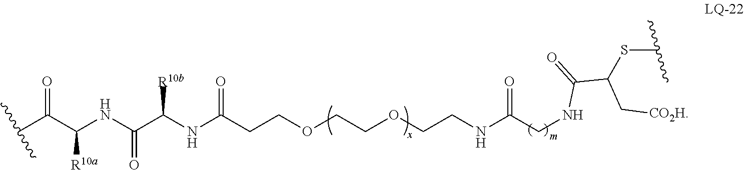

In another embodiment, -L-Q- is:

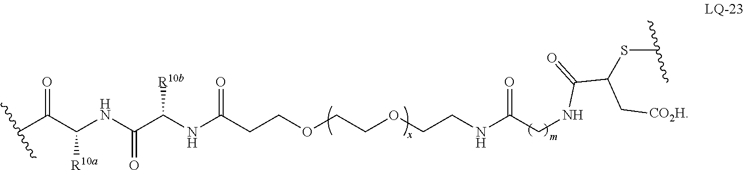

In some embodiments, -L-Q- is:

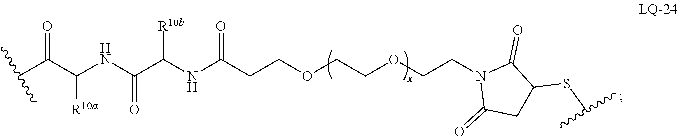

x is 0, 1, 2, 3, 4, 5, 6, 7, 8, 9, 10, 11, 12, 13, 14, or 15; and R^{10a} and R^{10b} are independently selected from the group consisting of hydrogen and optionally substituted C₁₋₆ alkyl.

In another embodiment, -L-Q- is:

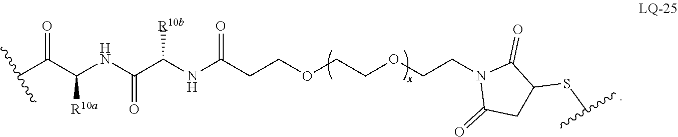

In another embodiment, -L-Q- is:

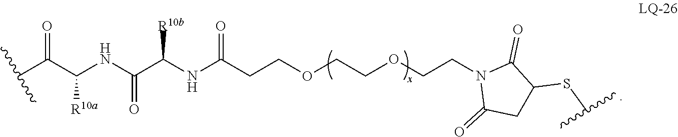

In another embodiment, -L-Q- is:

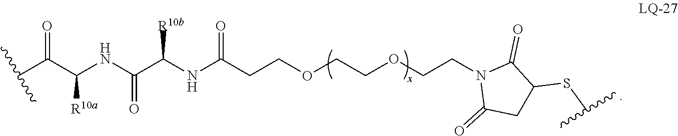

In another embodiment, -L-Q- is:

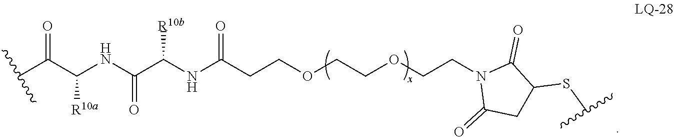

In some embodiments, -L-Q- is:

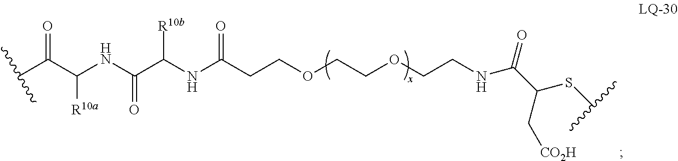

x is 0, 1, 2, 3, 4, 5, 6, 7, 8, 9, 10, 11, 12, 13, 14, or 15; and R^{10a} and R^{10b} are independently selected from the group consisting of hydrogen and optionally substituted C₁₋₆ alkyl.

In another embodiment, -L-Q- is:

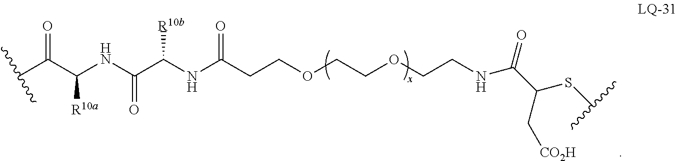

In another embodiment, -L-Q- is:

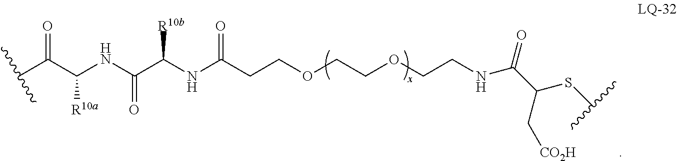

In another embodiment, -L-Q- is:

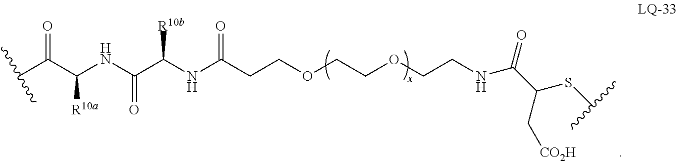

In another embodiment, -L-Q- is:

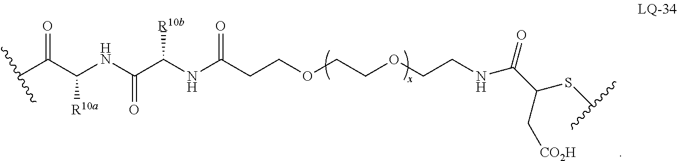

In some more specific embodiments, -L-Q- is selected from the group consisting of:

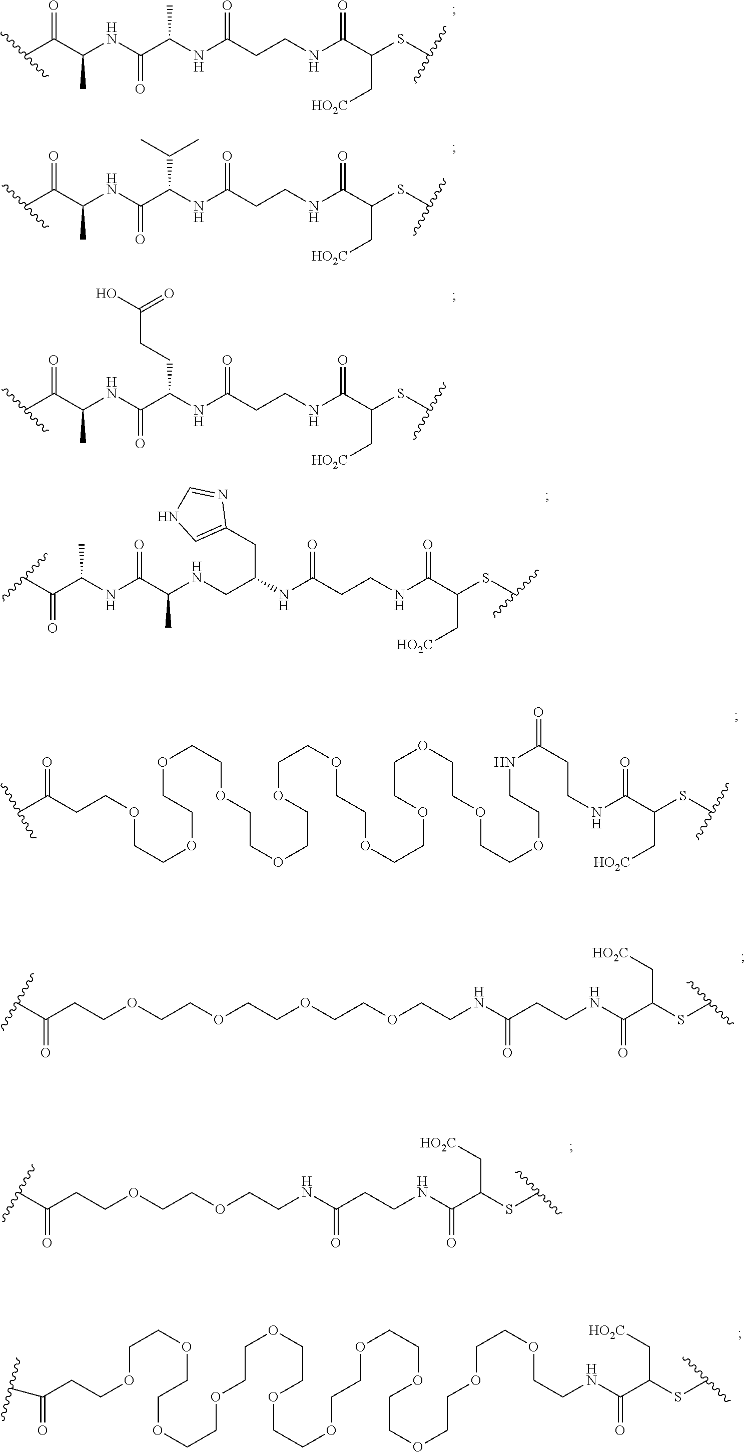

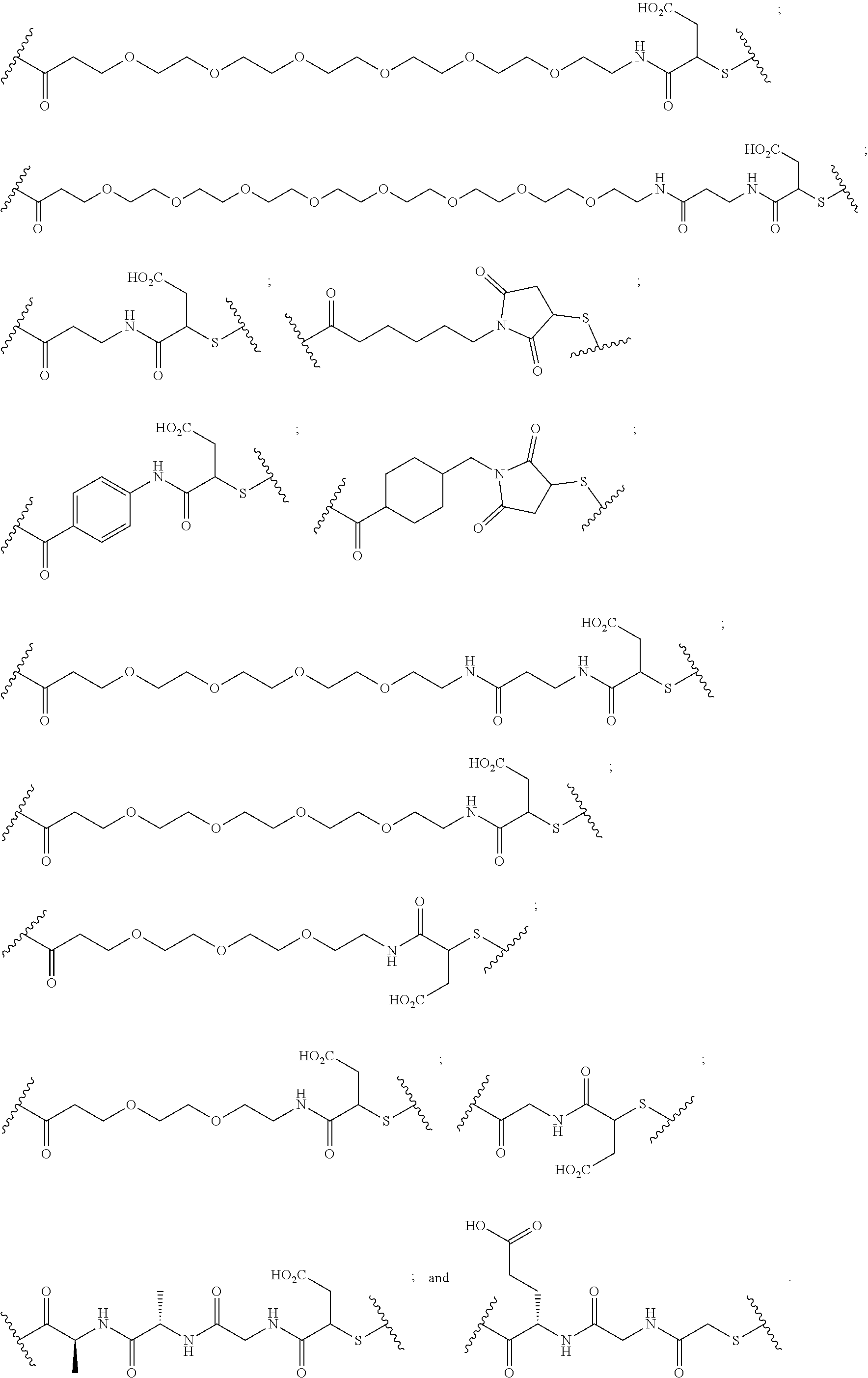

7. Category VII Linkers

Exemplary linkers are described in some detail in, for example: PCT Publication No. 2019/217591, the linkers of which are incorporated by reference herein in their entirety. In some specific embodiments, the linker has the following structure:

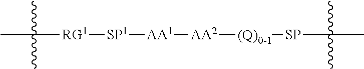

wherein:
SP¹ is absent, or a spacer; RG¹ is a reactive group residue; AA¹ is absent, or a divalent or trivalent linker comprising an amino acid residue which is optionally bonded directly or indirectly to a group HG; AA² is absent, or a dipeptide, tripeptide, or tetrapeptide residue; Q, when present, is a connector group residue; SP is absent, or a spacer; and HG, when present, is a hydrophilic group.

In some more specific embodiments, the linker has the following structure:

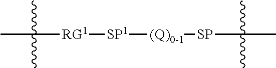

wherein:
SP¹ is absent, or a spacer; RG¹ is a reactive group residue; Q, when pre is

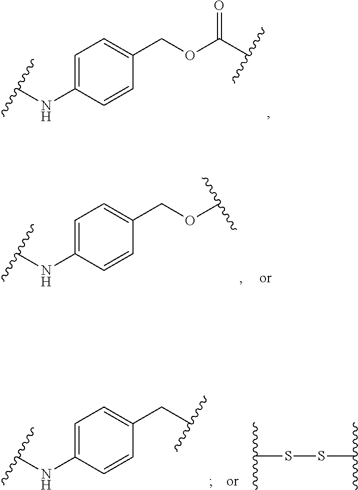

and
SP is absent, or a spacer; wherein the

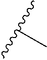

indicates the atoms through which the referenced group is bonded to the adjacent groups in the formula.

In some embodiments, SP¹ is absent, or a spacer;
RG¹ is a reactive group residue; AA¹ is absent, or a divalent or trivalent linker comprising an amino acid residue which is optionally bonded directly or indirectly to a group HG; AA¹ is absent, or a dipeptide, tripeptide, or tetrapeptide residue; Q, when present,

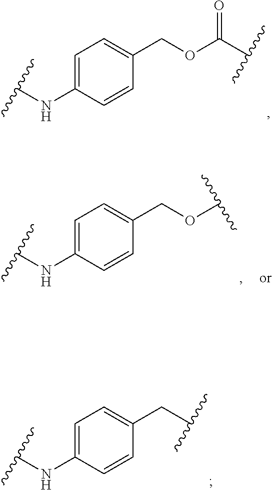 [SMILES: *Nc1ccc(COC(*)=O)cc1 ; *Nc1ccc(CO*)cc1 ; *Cc1ccc(N*)cc1]

SP is absent, or a spacer; and
HG, when present, is

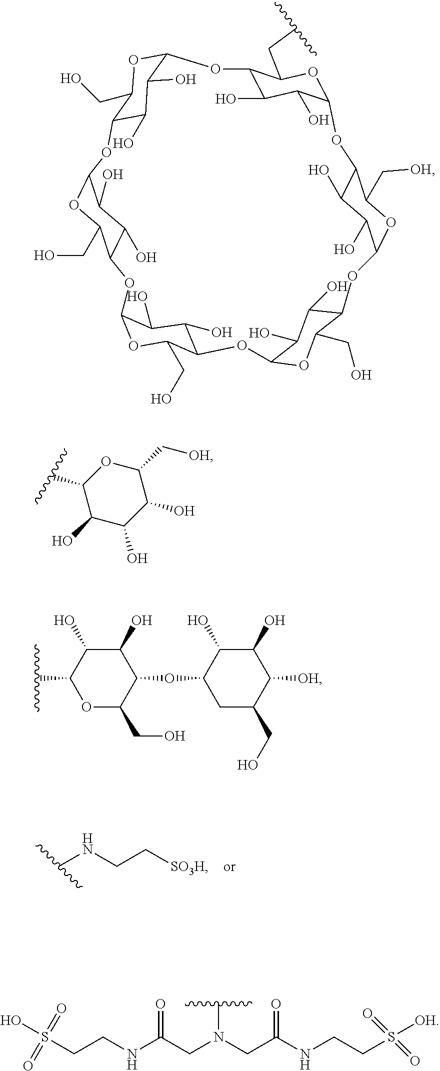 [SMILES: *CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O ; *[C@@H]1O[C@H](CO)[C@H](O)[C@H](O)[C@H]1O ; *[C@H]1O[C@H](CO)[C@@H](O[C@H]2C[C@H](CO)[C@@H](O)[C@H](O)[C@H]2O)[C@H](O)[C@H]1O ; *NCCS(=O)(=O)O ; *N(CC(=O)NCCS(=O)(=O)O)CC(=O)NCCS(=O)(=O)O]

wherein the

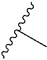

indicates the atoms through which the referenced group is bonded to the adjacent groups in the formula.

In some embodiments, AA¹-AA² has the following structure:

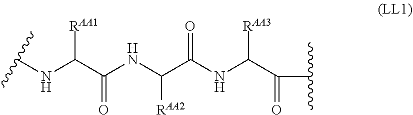

wherein R^{AA1}, R^{AA2}, and R^{AA3} are each, independently, amino acid side chains, at least one of which is bonded to (RG²)-SP²-HG, (RG²)-HG or HG; wherein the

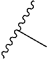

indicates the atoms through which AA¹-AA² is bonded to the adjacent groups in the formula. In some of such embodiments, R^{AA1} is a lysine, glutamine, glutamic acid or aspartic acid side chain bonded directly or indirectly to HG, and R^{AA2} and R^{AA3} are either valine and alanine or valine and citrulline sidechains respectively.

In some more specific embodiments, AA¹-AA² has one of the following structures:

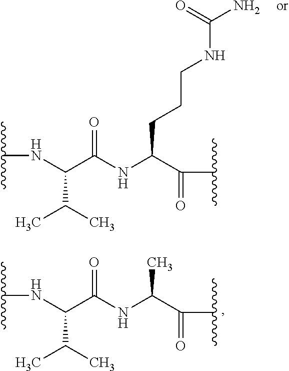

wherein the

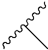

indicates the atoms through which AA¹-AA² is bonded to the adjacent groups in the formula.

In more specific embodiments, the RG¹ and RG² residues are independently, in each instance, selected from the group consisting of:

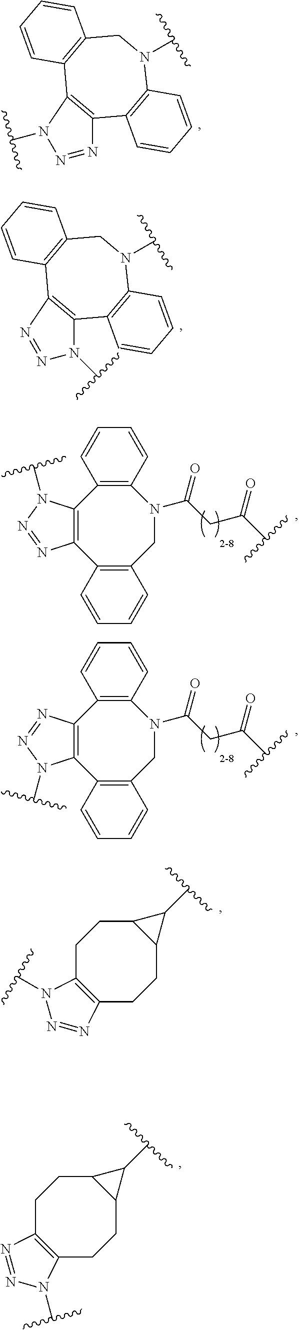

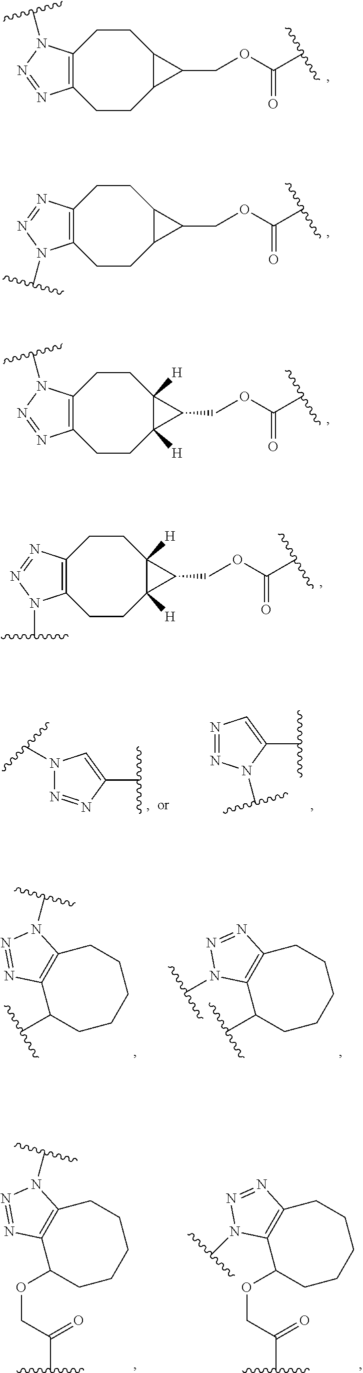

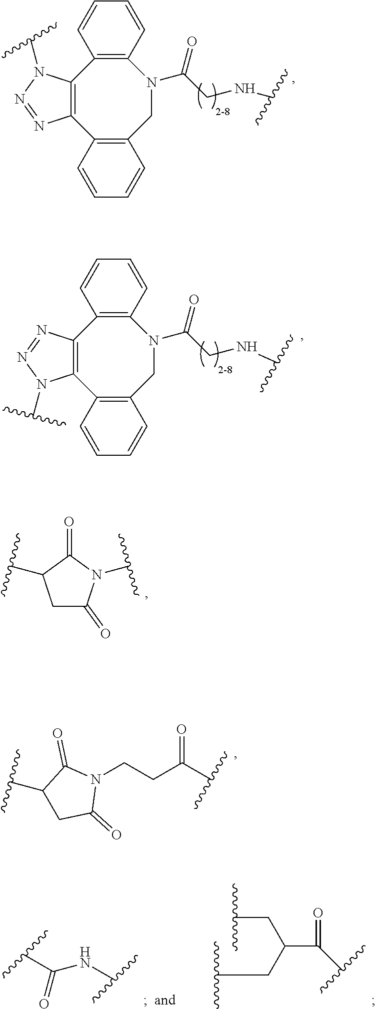

wherein the

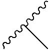

indicates the atom through which the RG¹ or RG² residue is bonded to the adjacent groups in the formula.

In some embodiments, SP, SP¹ and SP² are independently, in each instance, absent, or selected from the group consisting of C₁₋₆ alkylene, —NH—, —S—, —O—, —C(O)—, (—CH₂—CH₂—O)ₑ, —NH—CH₂—CH₂— (—O—CH₂—CH₂)ₑ—C(O)—, —C(O)—(CH₂)ᵤ—C(O)—, —C(O)—NH—(CH₂)ᵥ—, (glycine)₄-serine, and combinations thereof, wherein subscript e is an integer from 0 to 4, subscript u is an integer from 1 to 8, and subscript v is an integer from 1 to 8.

In some embodiments, n is an integer from 1 to 10, from 1 to 8, from 1 to 6, from 1 to 4, or n is 1, 2, 3, or 4.

In some embodiments, the linker has one of the following structures:

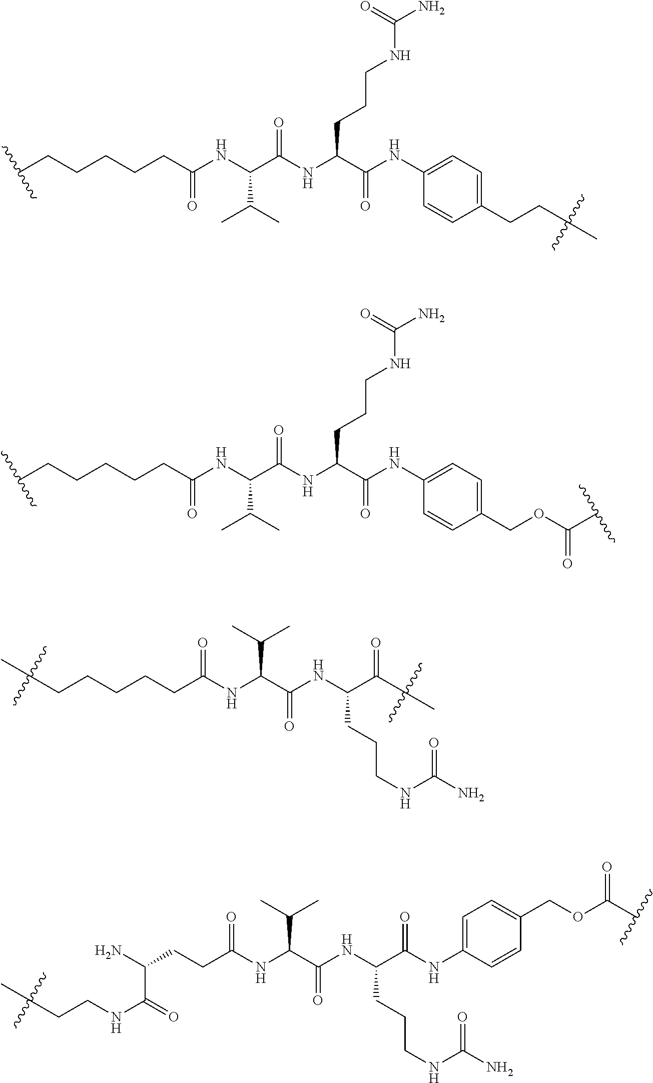 [SMILES: *CCCCCC(=O)N[C@H](C(=O)N[C@@H](CCCNC(N)=O)C(=O)Nc1ccc(CC*)cc1)C(C)C ; *CCCCCC(=O)N[C@H](C(=O)N[C@@H](CCCNC(N)=O)C(=O)Nc1ccc(COC(*)=O)cc1)C(C)C ; *CCCCCC(=O)N[C@H](C(=O)N[C@@H](CCCNC(N)=O)C(*)=O)C(C)C ; *CCNC(=O)[C@H](N)CCC(=O)N[C@H](C(=O)N[C@@H](CCCNC(N)=O)C(=O)Nc1ccc(COC(*)=O)cc1)C(C)C]

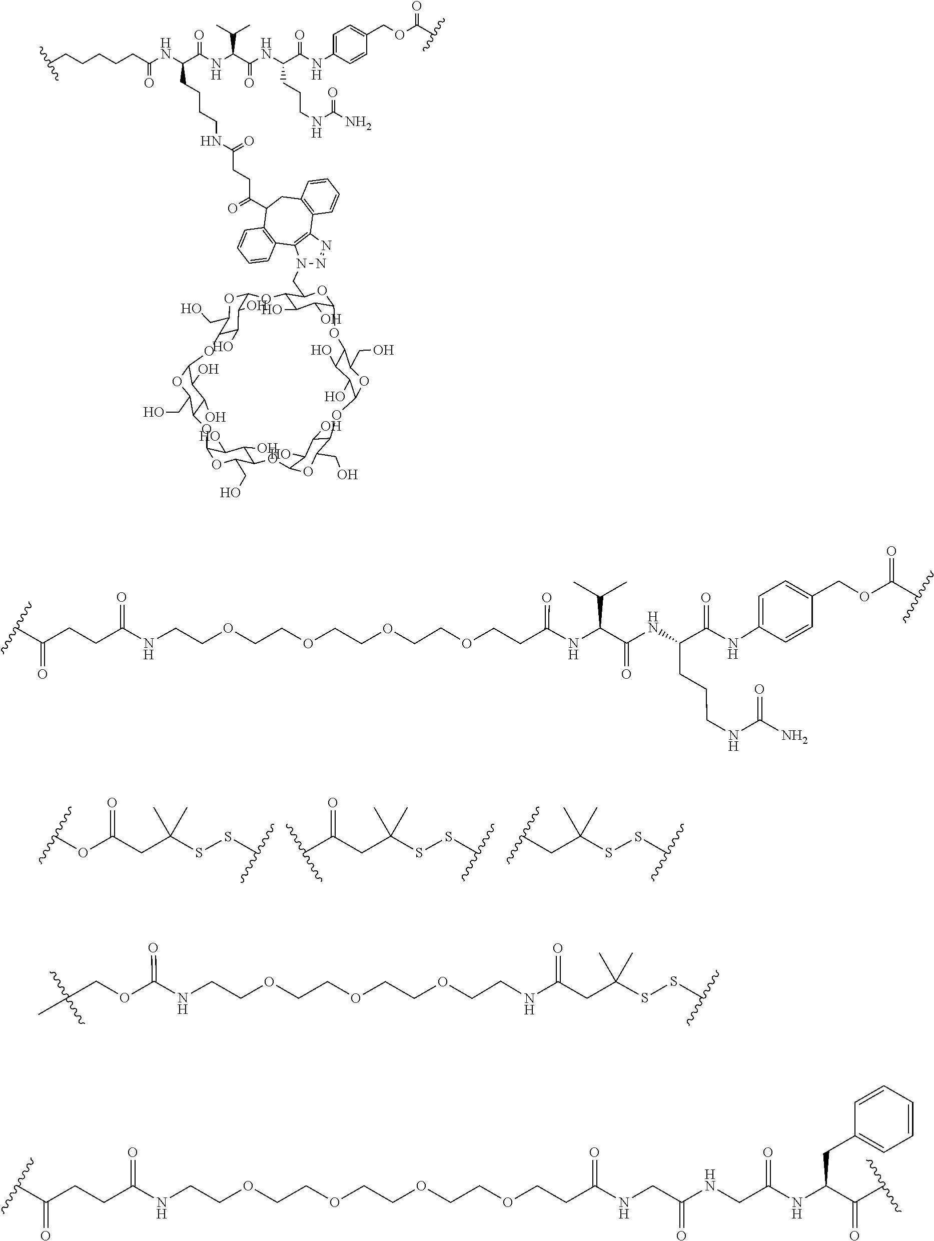

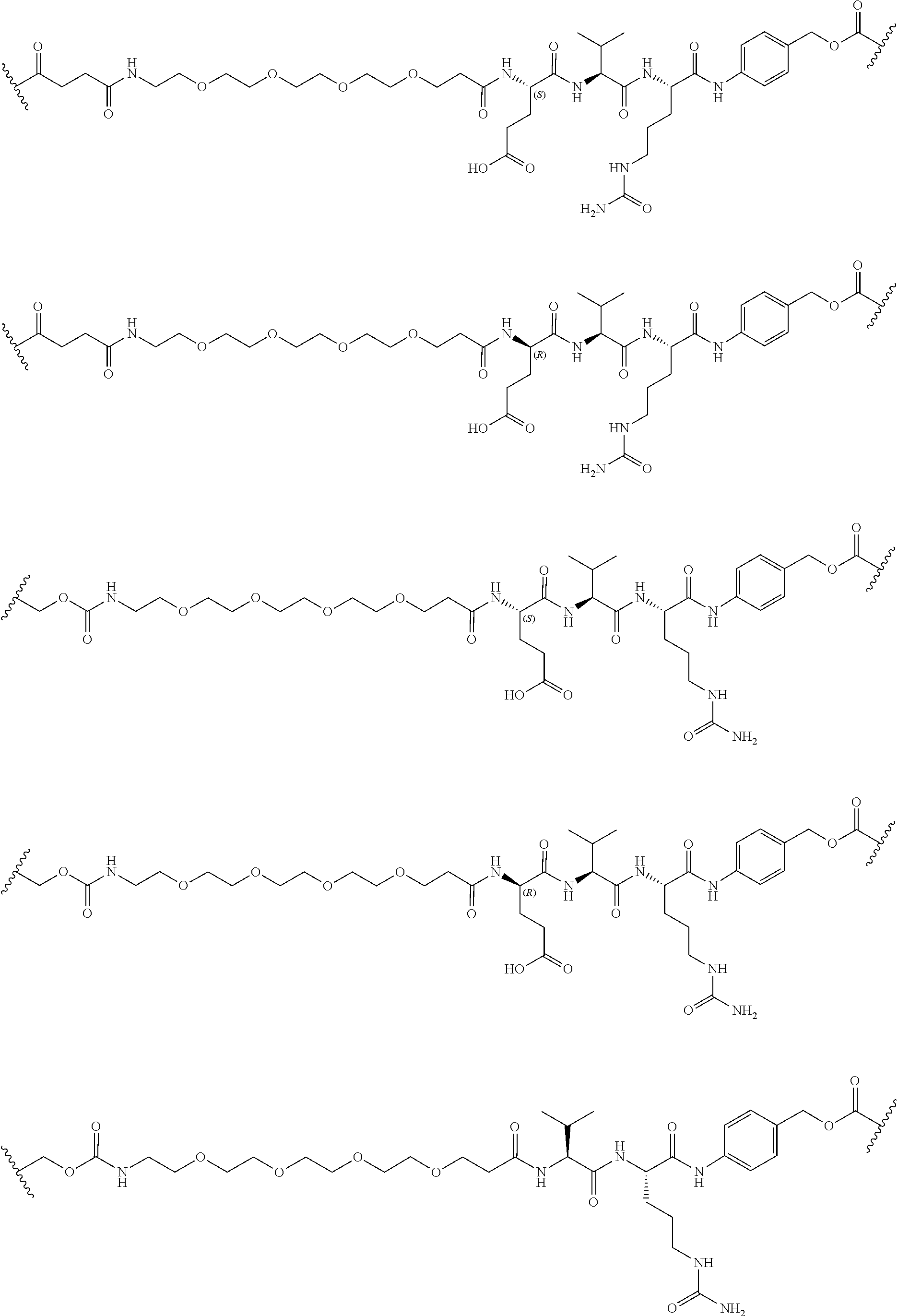

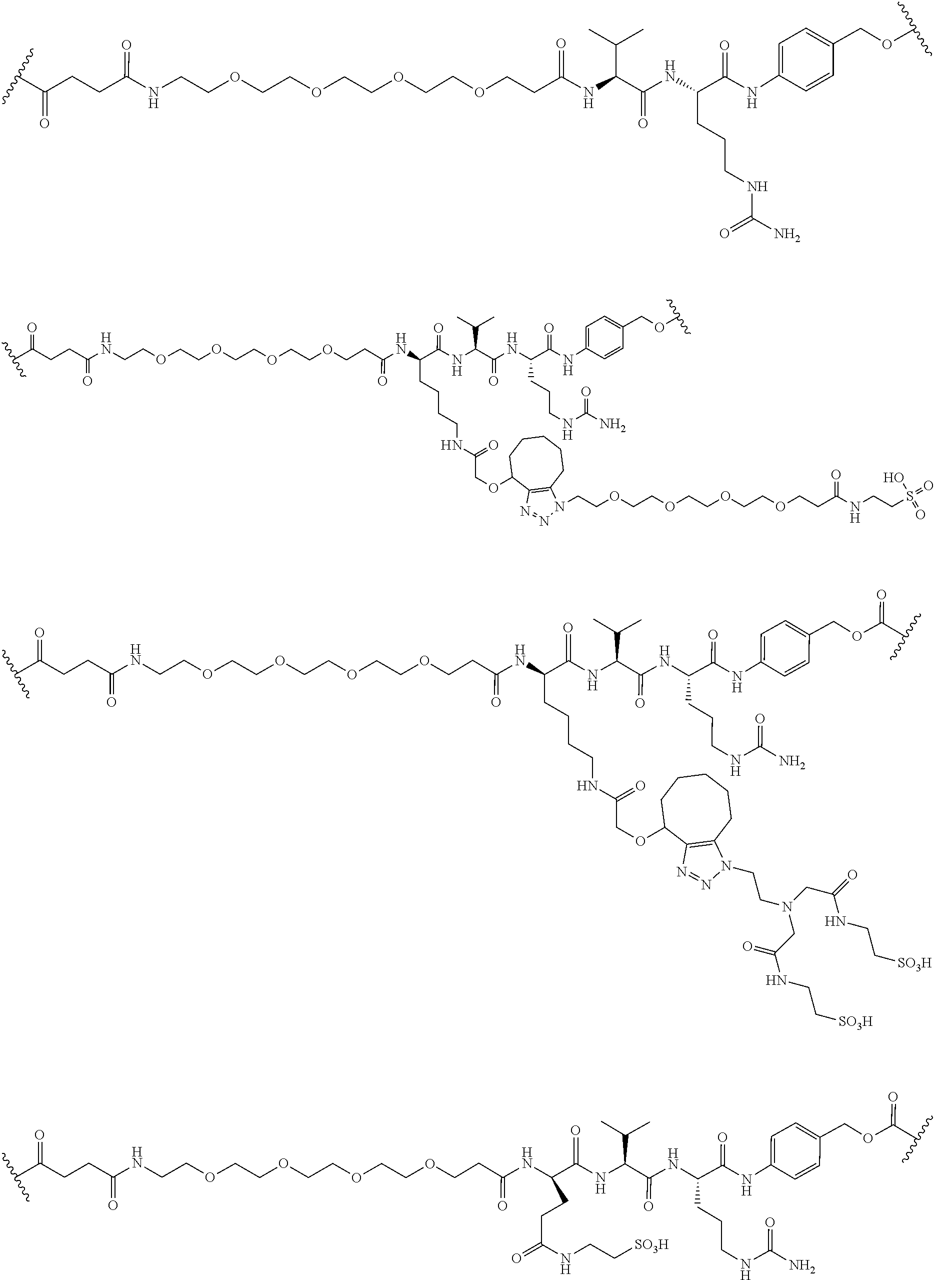

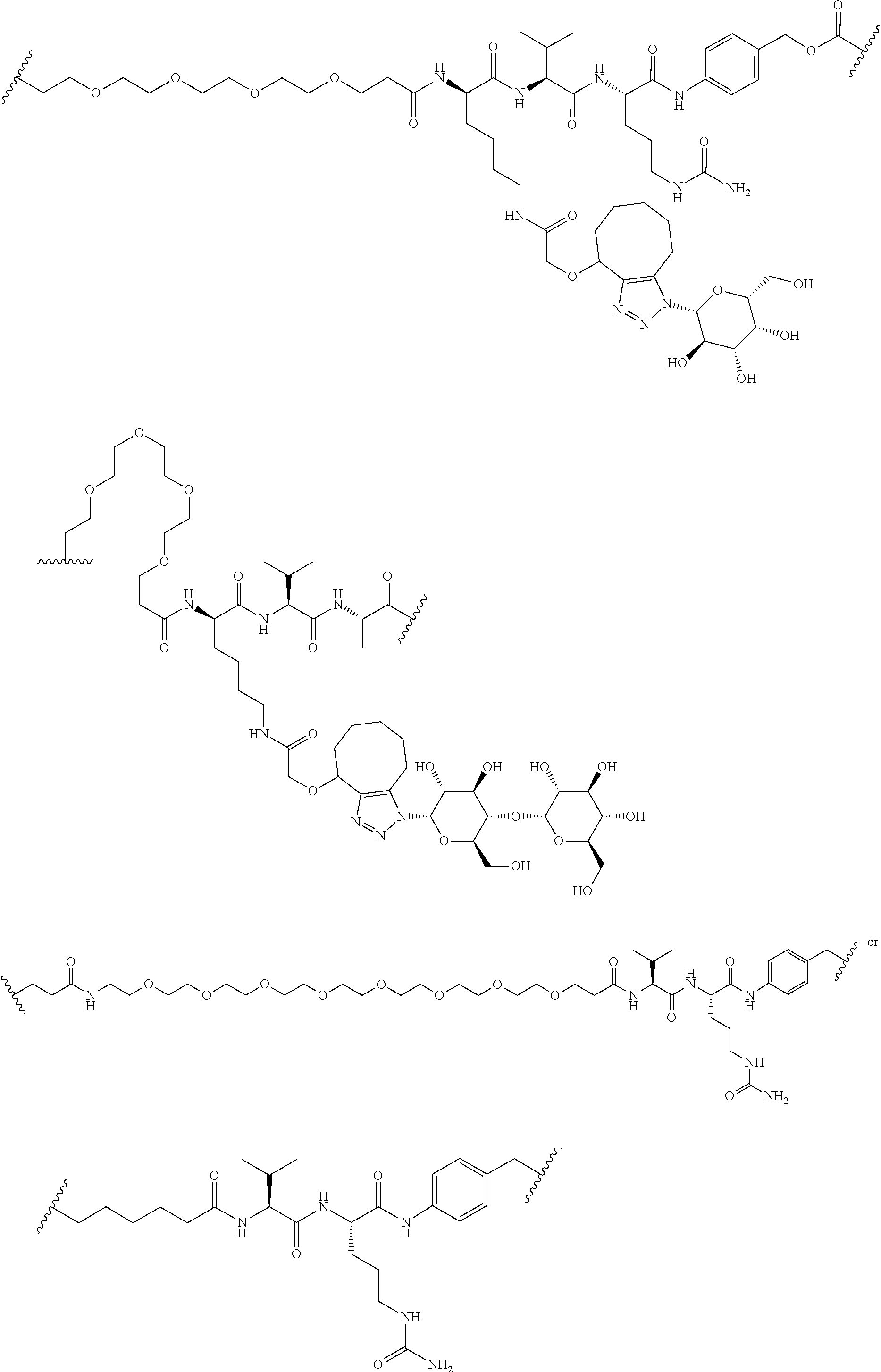

In some embodiments, the linker has the following structure: -L-SP-, wherein SP is —C(O)—C₁-C₁₀-alkylene-C(O)—, —C(O)—N(C₁₋₆alkyl)-C₁-C₁₀-alkylene-X¹— where X¹ is attached to L, —C(O)—N(H)—(C₁-C₁₀-alkylene)-S— where S is attached to L, —C(O)—N(C₁₋₆alkyl)-(C₁-C₁₀-alkylene)-S— where S is attached to L;

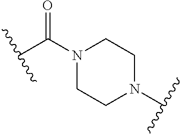 [SMILES: *C(=O)N1CCN(*)CC1]

where the point of attachment on the right-hand side (i.e., at N) is attached to L, —CH₂—NH— where the N is attached to L,

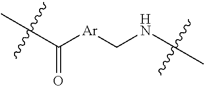

where the N is attached to L and where Ar is optionally substituted arylene or optionally substituted heteroarylene, —(C₁-C₁₀-alkylene)-NR⁵⁰C(O)—(C₁-C₁₀-alkylene)-NR^{50a}— where NR^{50a} is attached to L, —C(O)—(C₁-C₁₀-alkylene)-NR⁵⁰C(O)—(C₁-C₁₀-alkylene)-NR^{50a}— where NR^{50a} is attached to L and where each C₁-C₁₀-alkylene is independently optionally substituted with one or more hydroxy, —C(O)—N(R³⁵)—C₁-C₁₀-alkylene-C(O)NH—X²— where X² is attached to L, or

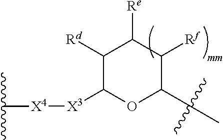

where X⁴ is attached to L; or SP is —C(O)—C₁-C₁₀-alkylene-C(O)—, —C(O)—N(C₁₋₆alkyl)-C₁-C₁₀-alkylene-X^{1b}— where X^{1b} is attached to L, —C(O)—N(H)—(C₁-C₁₀-alkylene)-X^{1b}— where X^{1b} is attached to L,

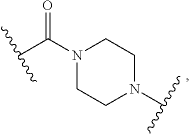 [SMILES: *C(=O)N1CCN(*)CC1]

where the point of attachment on the right-hand side (i.e., at N) is to L, —CH₂—NH— where the N is attached to L,

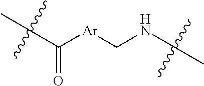

where the N is attached to L and where Ar is optionally substituted arylene or optionally substituted heteroarylene, —(C₁-C₁₀-alkylene)-NR⁵⁰C(O)—C₁-C₁₀-alkylene)-NR^{50a}— where NR^{50a} is attached to L, —C(O)—(C₁-C₁₀alkylene)-NR^{50a}— where NR^{50a} is attached to L and where each C₁—C₁₀-alkylene is independently optionally substituted with one or more hydroxy, —C(O)—N(R³⁵)—(C₁-C₁₀-alkylene)-C(O)NH—X²— where X² is attached to L, or

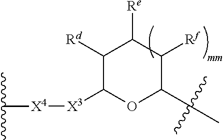

where X⁴ is attached to L; and X¹ is —N(C₁₋₆alkyl)-; X^{1b} is —S—, —NH—, or —N(C₁₋₆alkyl)-; X² is NH—; X³ is —CH₂—, X³ is —CH₂—O-(C₁-C₁₀-alkylene)-C(O)— where the C(O) is attached to X⁴, or X³ is —C(O)—; X⁴ is —O—; R³⁵ is H, —OH, —OCH₃, or C₁₋₆alkyl; R⁵⁰ and R^{50a} are independently hydrogen or C₁-C₆-alkyl; R^{d}, R^{e}, and R^{f} are independently —H, —OH, hydroxyalkyl, alkoxycarbonyl, —C(O)OH, or —CH₂OR^{g}, where each R^{g} is independently —CH₂C(O)OH or —CH₂C(O)O(alkyl); and mm is 0 or 1; n is an integer selected from 1-30, inclusive; and L is a linker.

In some embodiments, the linker comprises:

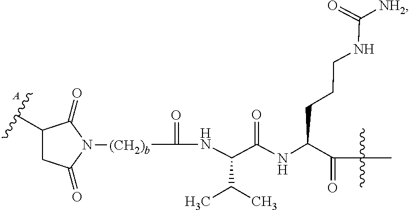

wherein b is an integer from 2 to 8 and

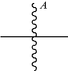

is a bond to the binding protein of this disclosure. In some embodiments, the linker comprises two reactive groups and can form bonds with, for example, thiols on two different chains of an antibody, or antigen binding fragment thereof:

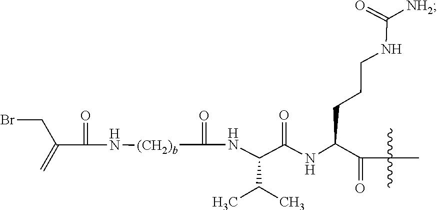

wherein b is an integer from 2 to 8. In further embodiments, the linker comprises two reactive groups and can form bonds with, for example, thiols on two different chains of an antibody, or antigen binding fragment thereof:

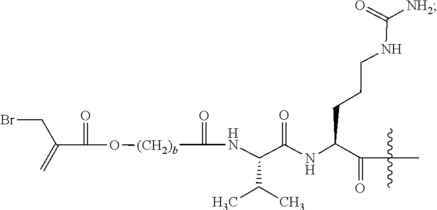

wherein b is an integer from 2 to 8. In some other embodiments, the linker comprises two reactive groups and can form bonds with, for example, thiols on two different chains of an antibody, or antigen binding fragment thereof:

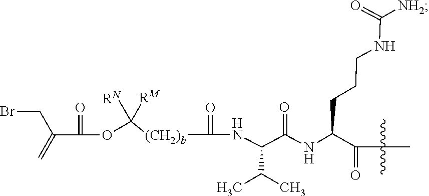

wherein b is an integer from 2 to 8, R^{N} is a hydrogen atom or alkyl, and R^{M} is alkyl. In additional embodiments, the linker comprises two reactive groups and can form bonds with, for example, thiols on two different chains of an antibody, or antigen binding fragment thereof:

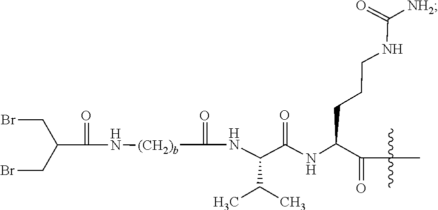

wherein b is an integer form 2 to 8. In certain embodiments, the linker comprises two reactive groups and can form bonds with, for example, thiols on two different chains of an antibody, or antigen binding fragment thereof:

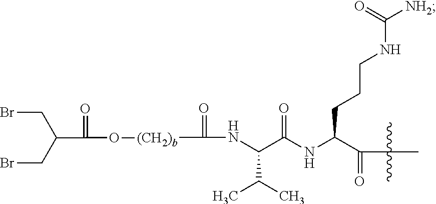

wherein b is an integer from 2 to 8. In some embodiments, the linker comprises two reactive groups and can form bonds with, for example, thiols on two different chains of an antibody, or antigen binding fragment thereof:

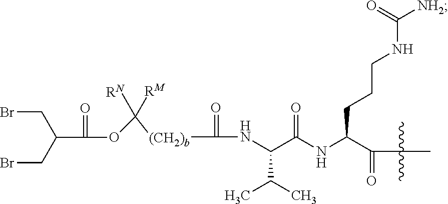

wherein b is an integer from 2 to 8; R^{N} is a hydrogen atom or alkyl; and R^{M} is alkyl.

Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference.

Generally, suitable linkers for the conjugates of the disclosure are those that are sufficiently stable to exploit the circulating half-life of the polypeptide and, at the same time, capable of releasing its payload (e.g., GR agonists) after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include acid-labile linkers, hydrolysis-labile linkers, enzymatically cleavable linkers, reduction labile linkers, self-immolative linkers/groups, and non-cleavable linkers. Suitable linkers also include those that are or comprise peptides, glucuronides, succinimide-thioethers, polyethylene glycol (PEG) units, hydrazones, mal-caproyl units, dipeptide units, valine-citrulline units, and para-aminobenzyl (PAB) units.

Any linker molecule or linker technology known in the art can be used to create or construct an ADC of the present disclosure. In some embodiments, the linker is a cleavable linker. According to other embodiments, the linker is a non-cleavable linker. Exemplary linkers that can be used in the context of the present disclosure include, linkers that comprise or consist of e.g., MC (6-maleimidocaproyl), MP (maleimidopropanoyl), val-cit (valine-citrulline), val-ala (valine-alanine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 21:5-13, and the references cited therein, the contents of which are incorporated by reference herein in their entireties.

In some embodiments, the linkers are stable in physiological conditions. In some embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include peptide bonds, ester linkages, hydrazones, and disulfide linkages. In some embodiments, the linker comprises a cathepsin-cleavable linker.

In some embodiments, the linker comprises a non-cleavable moiety.

Suitable linkers also include those that are chemically bonded to two cysteine residues of a single binding protein of this disclosure.

In some embodiments, the linker comprises one or more amino acids. Suitable amino acids include natural, non-natural, standard, non-standard, proteinogenic, non-proteinogenic, and L- or D-′-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In some embodiments, one or more side chains of the amino acids is linked to a side chain group, described below. In some embodiments, the linker comprises valine and citrulline. In some embodiments, the linker comprises lysine, valine, and citrulline. In some embodiments, the linker comprises lysine, valine, and alanine. In some embodiments, the linker comprises valine and alanine.

In some embodiments, the linker comprises a self-immolative group. The self-immolative group can be any such group known to those of skill. In particular embodiments, the self-immolative group is p-aminobenzyl (PAB), or a derivative thereof. In some embodiments, the self-immolative group is p-aminobenzyloxy. In some embodiments the self-immolative group comprises a cleavable di-sulfide group. Useful derivatives include p-aminobenzyloxycarbonyl (PABC). Those of skill will recognize that a self-immolative group is capable of carrying out a chemical reaction which releases the remaining atoms of a linker from a payload (e.g., a GR agonist).

In some embodiments, the linker is

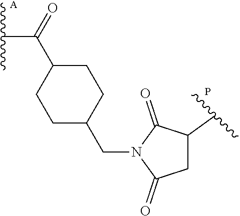

wherein

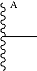

is a bond to a binding protein of this disclosure, e.g., via lysine residue, and

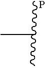

is a bond to the payload (i.e., a GR agonist). In some embodiments, the linker is:

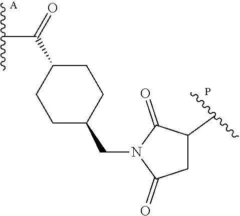

wherein

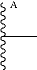

is a bond to a binding protein of this disclosure, e.g., via lysine residue, and

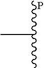

is a bond to the payload (i.e., a GR agonist).

In some embodiments, the linker is:

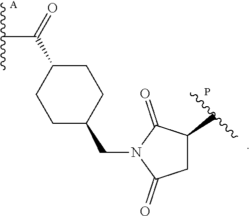

In some embodiments, the linker is:

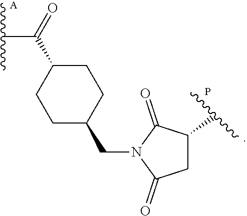

In some embodiments, the linker is derived from maleimidylmethyl-4-trans-cyclohexanecarboxysuccinate:

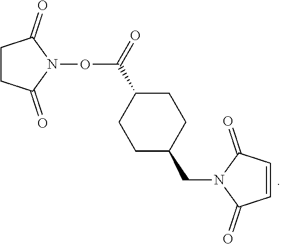 [SMILES: O=C1C=CC(=O)N1C[C@H]1CC[C@H](C(=O)ON2C(=O)CCC2=O)CC1]

In some embodiments, the linker is:

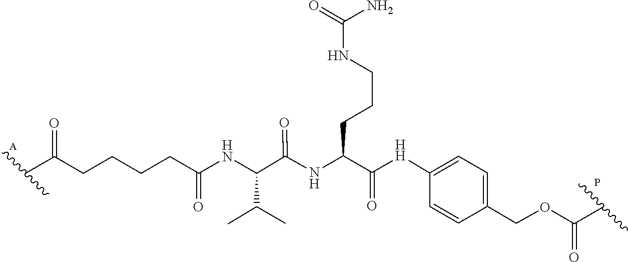

wherein

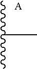

is a bond to a binding protein of this disclosure, e.g., via lysine residue, and

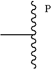

is a bond to the payload (i.e., a GR agonist).

In some embodiments, L is a cleavable linker. In some embodiments, L is a non-cleavable linker. In some embodiments, L comprises a dipeptide. In some embodiments, L comprises a PAB moiety. In some embodiments, L comprises a disulfide moiety.

In some embodiments, L comprises a moiety having the following structure:

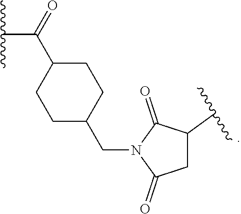 [SMILES: *C(=O)C1CCC(CN2C(=O)CC(*)C2=O)CC1]

In some embodiments, L comprises a moiety having the following structure:

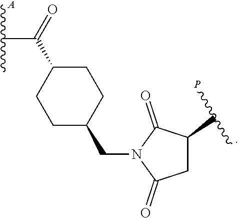

In some embodiments, L comprises a moiety having the following structure:

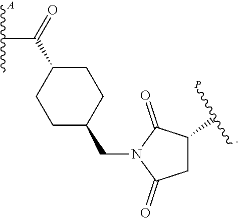

In some embodiments, L comprises a moiety having the following structure:

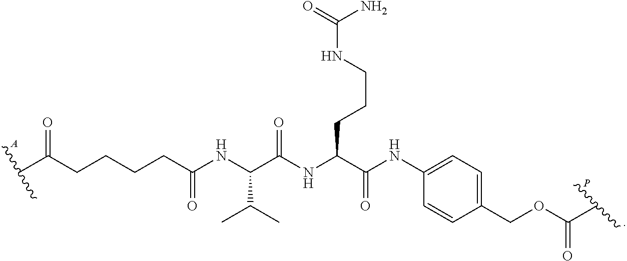

In some embodiments, the linker comprises a cyclodextrin group. In some embodiments, the linker provides a compound with the following structure:

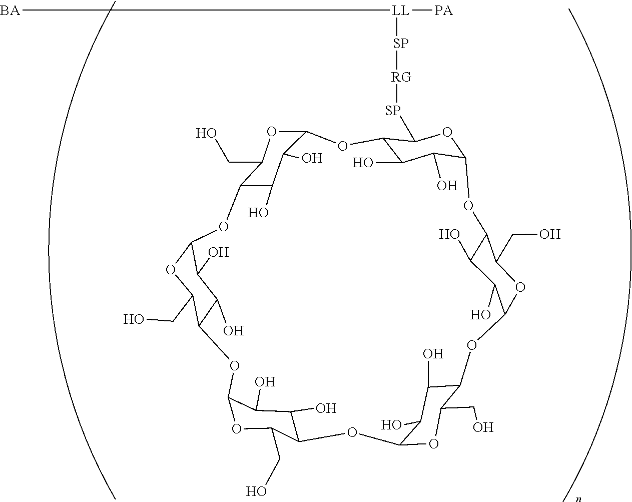

wherein BA is a binding protein of this disclosure, LL is a trivalent linker, RG is a reactive linker residue, SP is, independently in each instance, absent or a spacer group, subscript n is an integer from 1 to 30; and PA is a payload (i.e., a GR agonist). In some embodiments, n is from 1 to 4. In some embodiments, n is 4. In some embodiments, n is 2. In some embodiments, n is 1. In some embodiments, n is 3.

In some embodiments, the linker comprises a cyclodextrin group. In some embodiments, the linker provides a compound having the following structure:

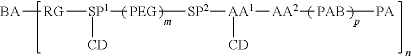

wherein BA is a binding protein of this disclosure; RG is a reactive group residue; SP¹ and SP² are each, independently in each instance, absent or a spacer group residue, and wherein SP¹ comprises a trivalent linker; AA¹ is a trivalent linker comprising an amino acid residue; AA² is a di-peptide residue; PEG is a polyethylene glycol residue, PAB is

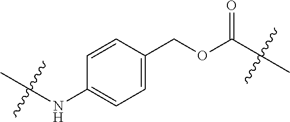 [SMILES: *Nc1ccc(COC(*)=O)cc1]

wherein the

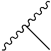

indicates the atom through which the PAB is bonded to the adjacent groups in the formula, CD is, independently in each instance, absent or a cyclodextrin residue, wherein at least one CD is present, subscript n is an integer from 1 to 30; subscript m is an integer from 0 to 5; subscript p is 0 or 1; and PA is a payload moiety (i.e., a GR agonist). In these examples, subscript m is 0, 1, 2, 3, 4, or 5. In some examples, subscript m is 0. In some examples, subscript m is 1. In some examples, subscript m is 2. In some examples, subscript m is 3. In some examples, subscript m is 4. In some examples, subscript m is 5. In some examples, subscript p is 0. In some examples, subscript p is 1. In some examples, any one of AA¹ or AA² comprises, independently in each instance, an amino acid selected from alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is an amino acid selected from alanine, valine, leucine, isoleucine, methionine, tryptophan, phenylalanine, proline, glycine, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is lysine. In some embodiments, AA¹ is lysine or a derivative of lysine. In some embodiments, the AA² is valine-citrulline. In some embodiments, the AA² is citrulline-valine. In some embodiments, the AA² is valine-alanine. In some embodiments, the AA² is alanine-valine. In some embodiments, the AA² is valine-glycine. In some embodiments, the AA² is glycine-valine. In some embodiments, the AA¹-AA² glutamine-valine-citrulline. In some embodiments, the AA¹-AA² is glutamine-valine-citrulline. In some embodiments, the AA¹-AA² is lysine-valine-alanine. In some embodiments, the AA¹-AA² is lysine-valine-citrulline. In some embodiments, the AA¹-AA² is glutamine-valine-citrulline. In some embodiments, the lysine is L-lysine. In some embodiments, the lysine is D-lysine. In some examples, SP¹ is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ— C(O)—, —C(O)—NH—(CH₂)ᵥ—, and combinations thereof, wherein subscript e is an integer from 0 to 4, subscript u is an integer from 1 to 8, and subscript v is an integer from 1 to 8. In some examples, SP² is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, and combinations thereof, wherein subscript e is an integer from 0 to 4, subscript u is an integer from 1 to 8, and subscript v is an integer from 1 to 8.

In some the linker is selected from:

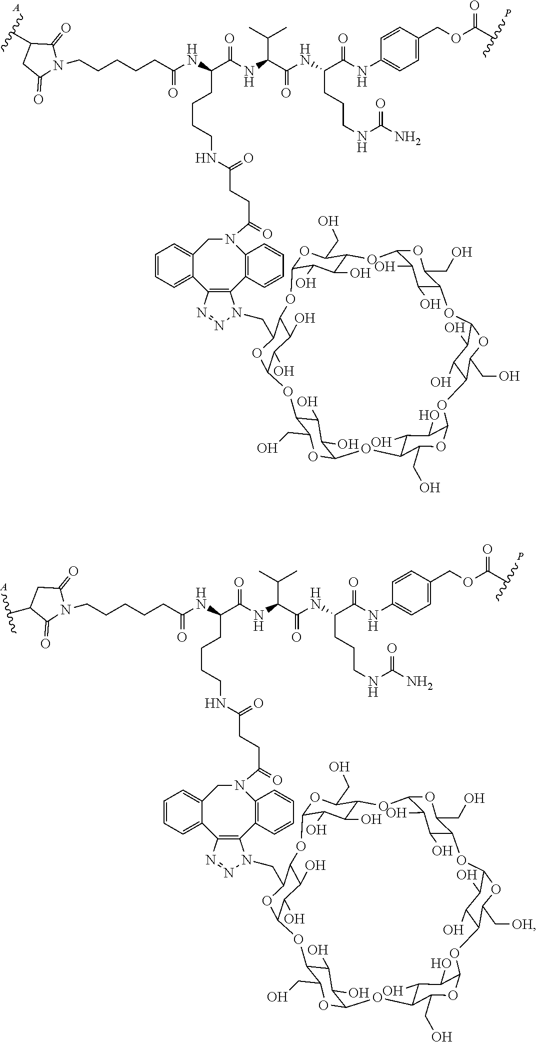

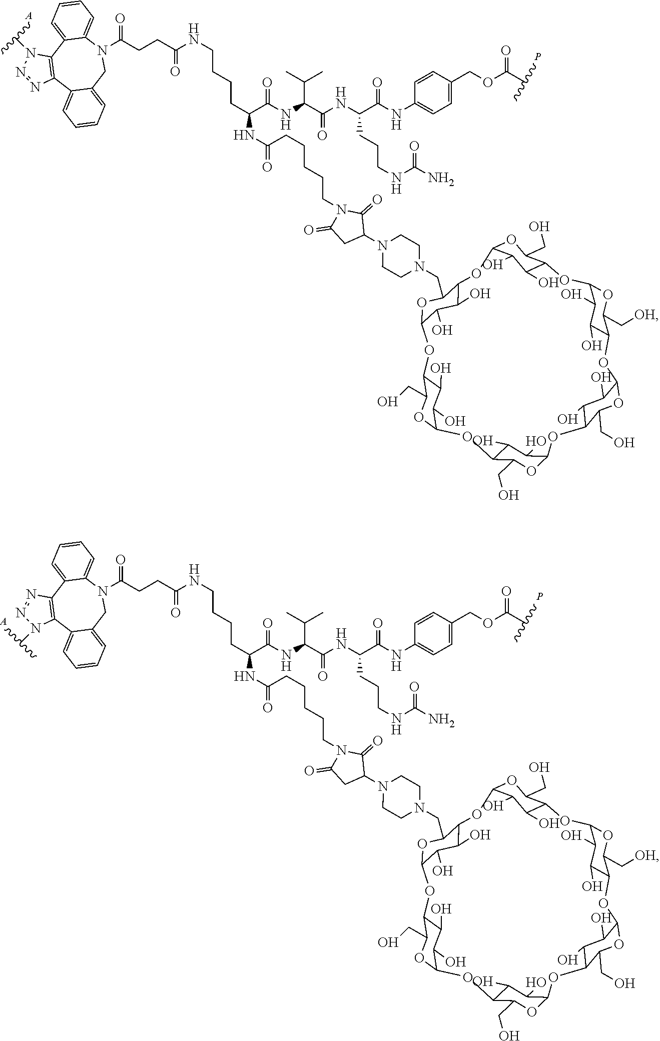

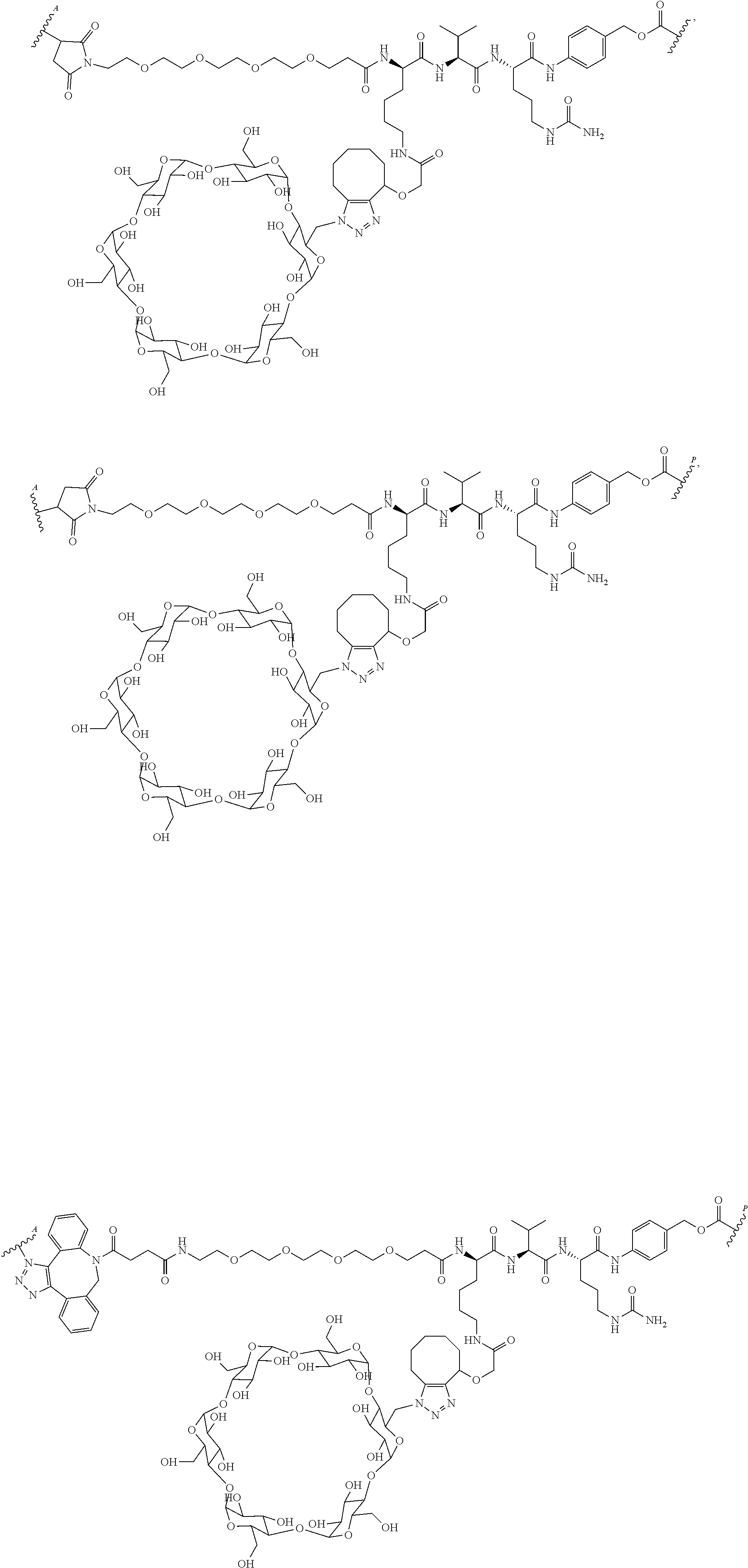

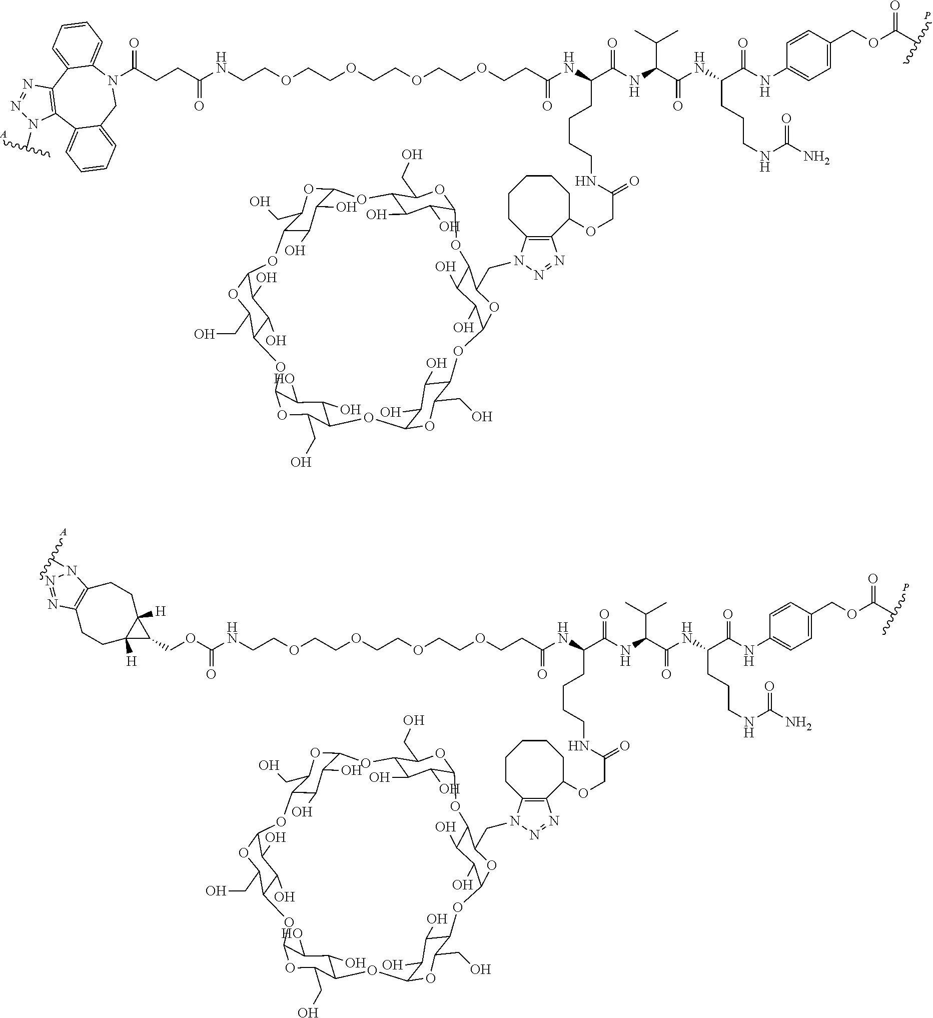

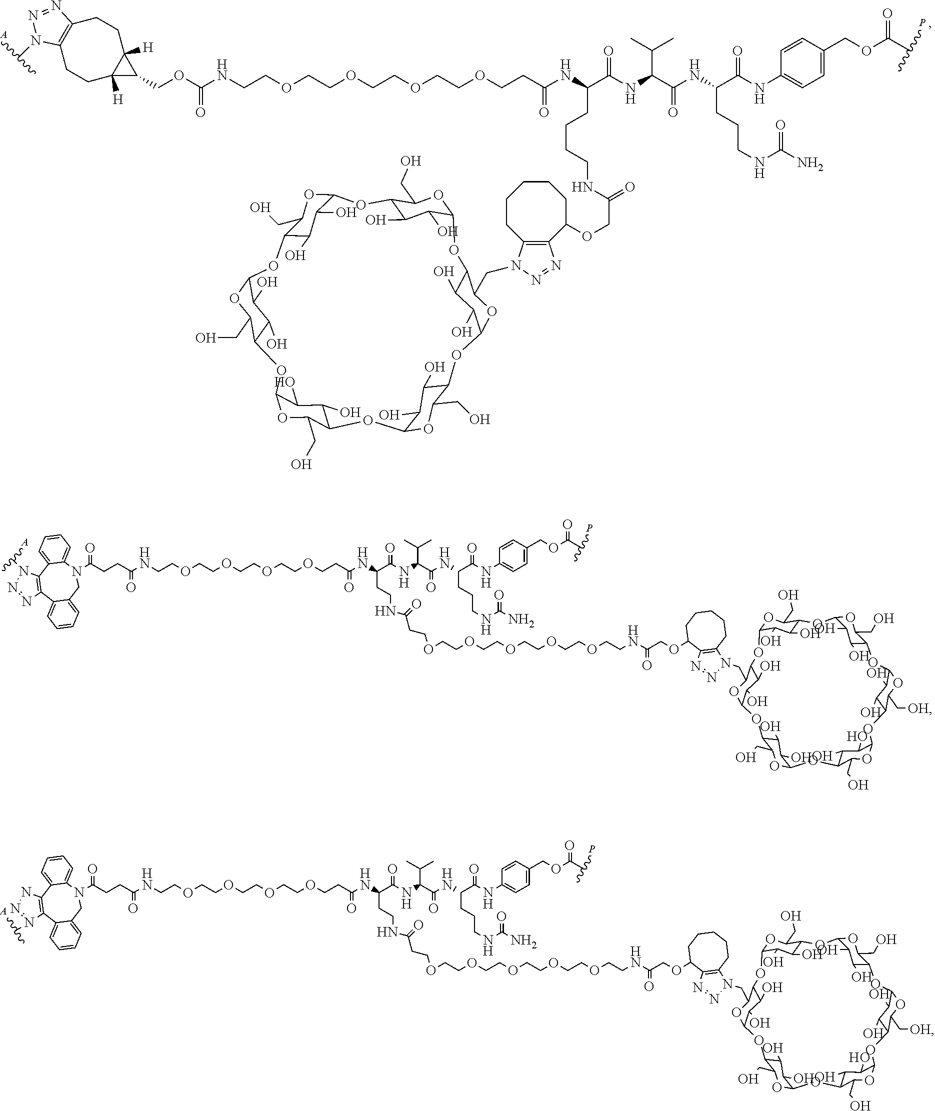

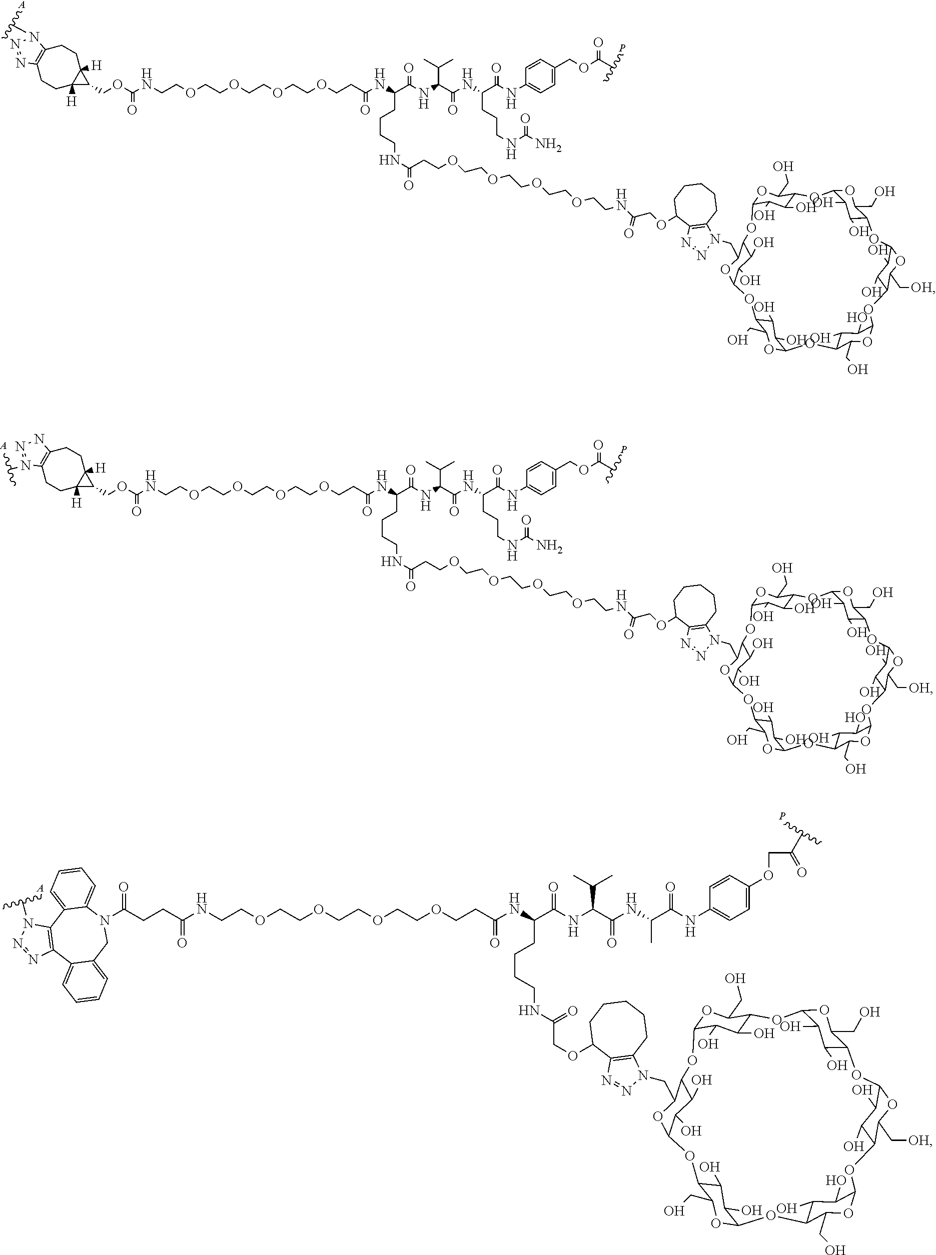

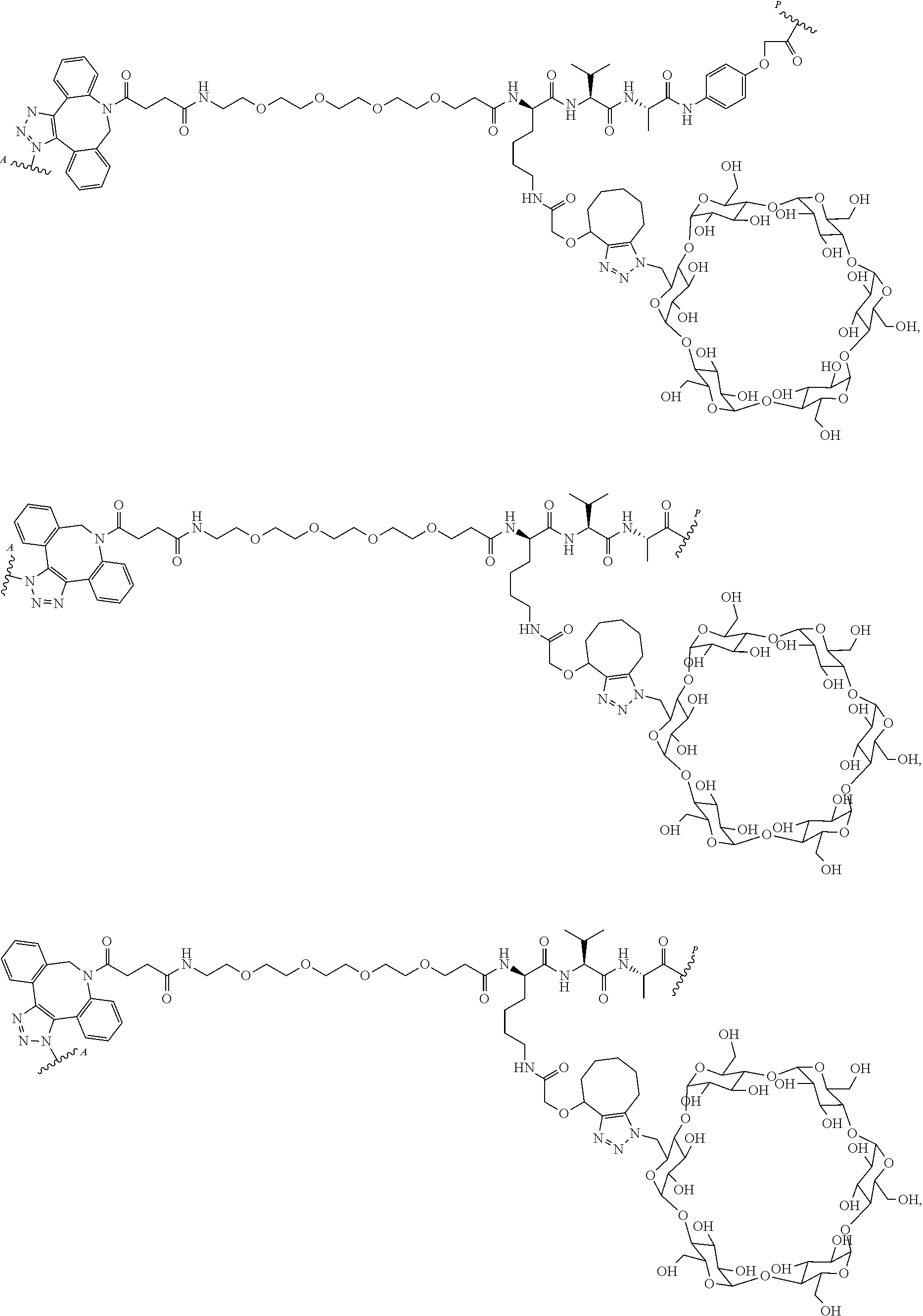

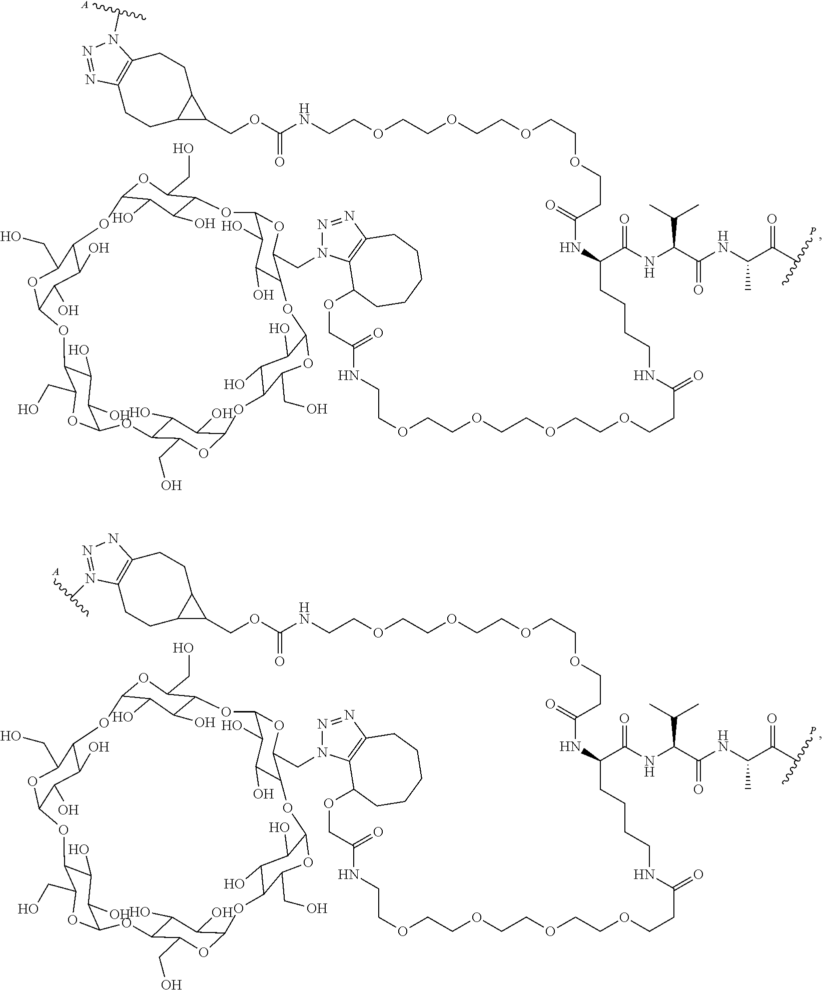

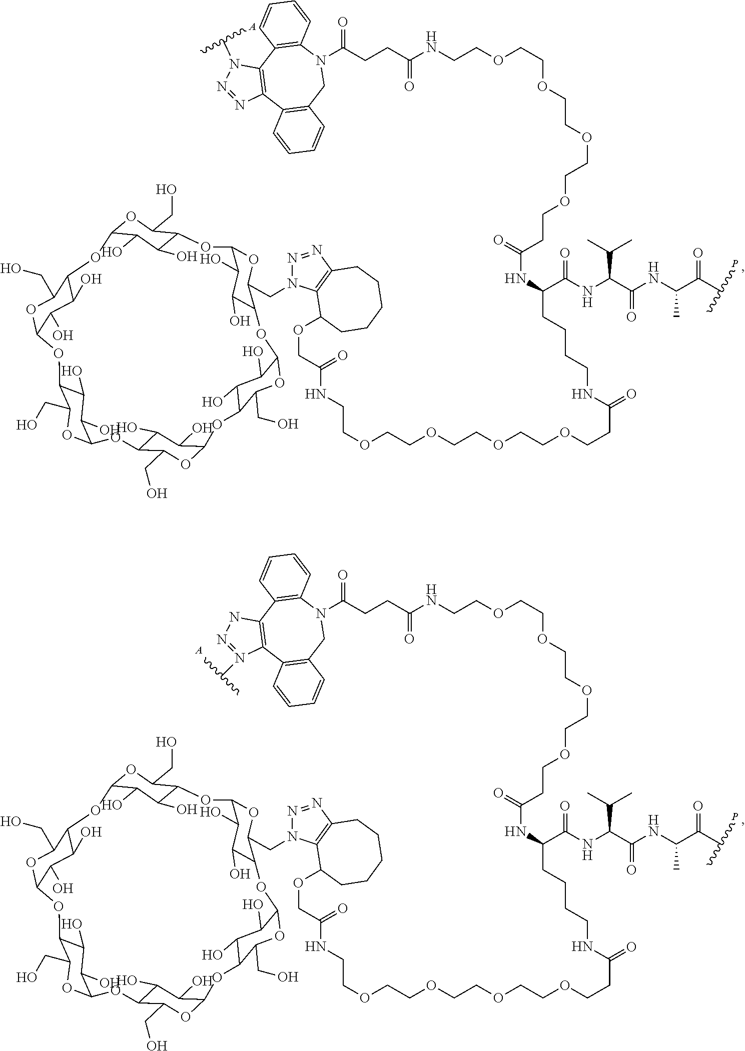

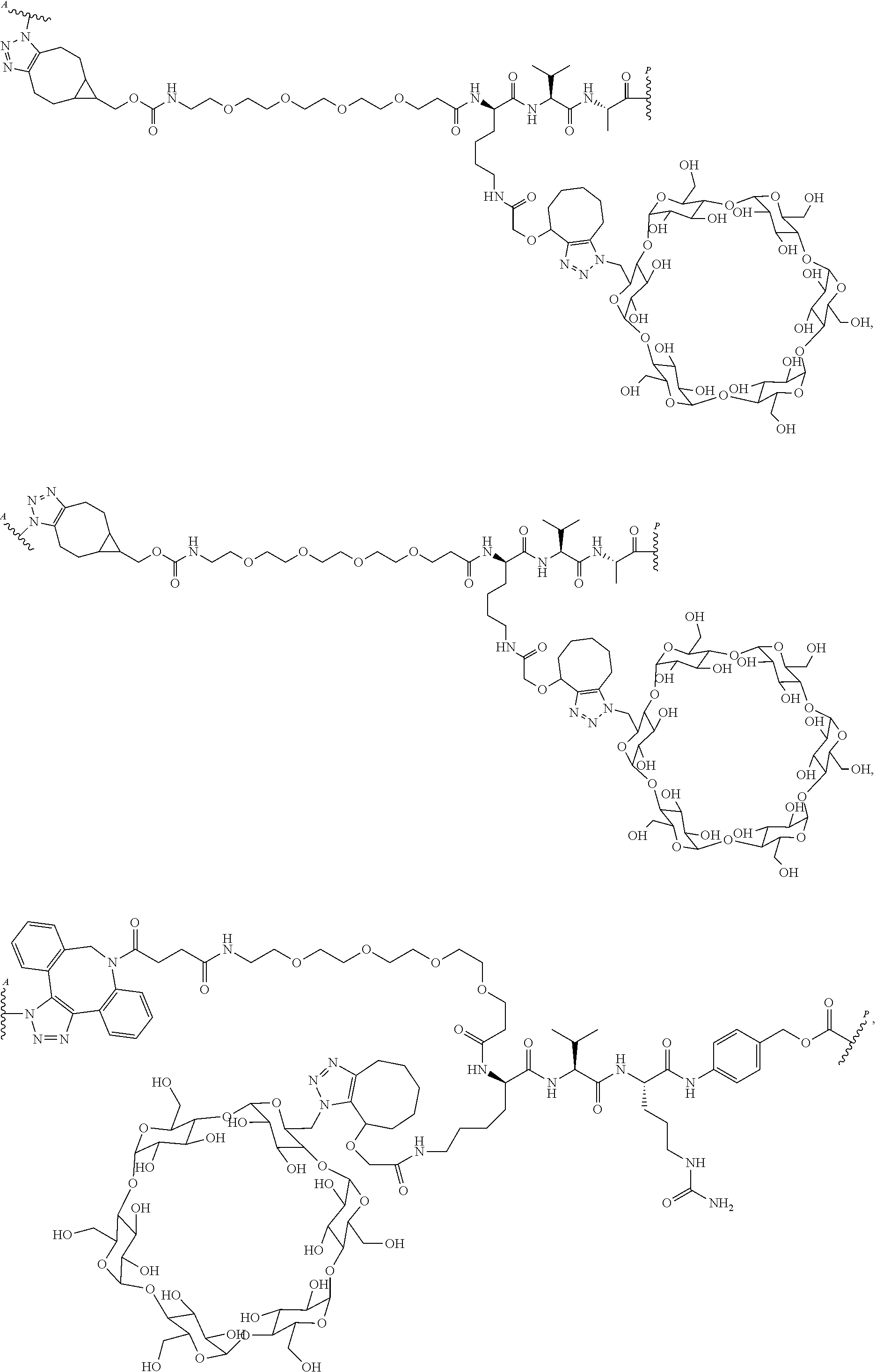

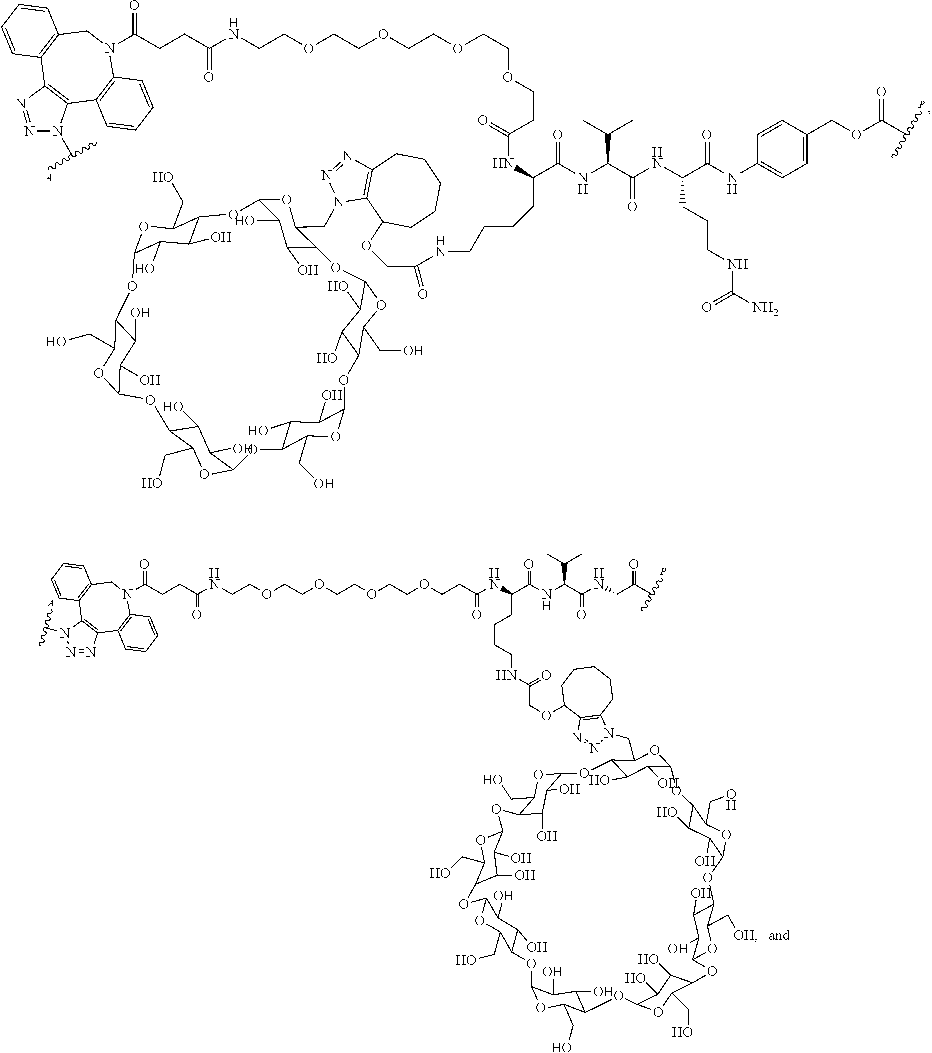

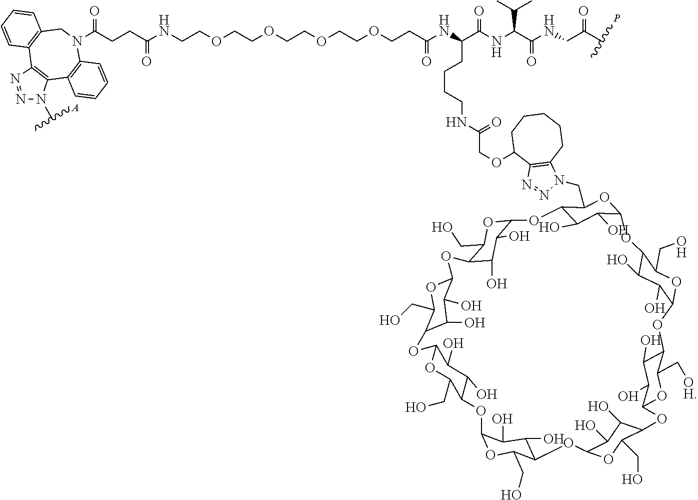

Also included in these examples, is a pharmaceutically acceptable salt, solvate, stereoisomeric form thereof, a regioisomer thereof, or mixture of regioisomers thereof, wherein each

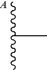

is a bond to a binding protein of this disclosure; and each

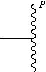

is a bond to the payload (i.e., a GR agonist).

In some embodiments, the linker comprises a terminal hydrophilic group (HG). In some embodiments, the linker comprises a taurine group. In some embodiments, the linker comprises a terminal sulfonic acid group. In some embodiments, the compound has the following structure:

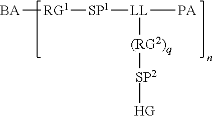

wherein, BA is a binding protein of this disclosure; LL is a trivalent linker; RG¹ and RG² are reactive group residues; SP¹ and SP² are independently, in each instance, absent, or a spacer group residue; HG is a hydrophilic residue; PA is a payload residue (i.e., a GR agonist); subscript n is an integer from 1 to 30; and subscript q is 0 or 1.

In some instances, more than one trivalent linker LL may be present. In some instances, n is an integer from 1 to 4. In some instances. n is 1. In some instances, n is 2. In some instances, n is 3. In some instances, n is 4. In some instances, HG is a terminal hydrophilic group. In some instances, HG comprises one terminal sulfonic acid group or a salt thereof. In other instances, HG comprises more than one terminal sulfonic acid groups or salts thereof. In some instances, HG comprises one terminal phosphonic acid group or a salt thereof. In other instances, HG comprises more than one terminal phosphonic acid groups or salts thereof. In some instances, HG comprises one terminal tertiary amine group or a salt thereof. In other instances, HG comprises more than one terminal tertiary amine groups or salts thereof. In some instances, HG comprises one terminal polyol (e.g., glucose, maltose) or a derivative thereof. In other instances, HG comprises more than one terminal polyol (e.g., glucose, maltose) or derivatives thereof.

In another example, the compound has the following structure:

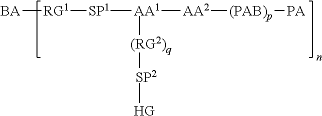

wherein BA, RG¹, SP¹, RG², SP² and HG are as defined above, AA¹ is a trivalent linker comprising an amino acid residue; AA² is a dipeptide residue; and PAB is wherein the

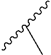

indicates the atom through which the PAB is bonded to the

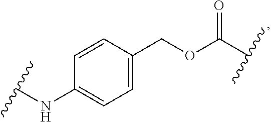 [SMILES: *Nc1ccc(COC(*)=O)cc1]

adjacent groups in the formula; subscript p is 0 or 1; and subscript q is 0 or 1.

In some instances, subscript p is 0 and subscript q is 0. In some instances, subscript p is 1; and subscript q is 0. In some instances, subscript p is 0; and subscript q is 1. In some instances, subscript p is 1; and subscript q is 1. In some instances, SP¹ comprises from 0-5 polyethylene glycol (PEG) residues. In some instances, SP² comprises from 0-5 PEG residues. In some examples, SP¹ is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, and combinations thereof, wherein subscript e is an integer from 0 to 4, subscript u is an integer from 1 to 8, and subscript v is an integer from 1 to 8. In some examples, SP² is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, and combinations thereof, wherein subscript e is an integer from 0 to 4, subscript u is an integer from 1 to 8, and subscript v is an integer from 1 to 8. In some examples, any one of AA¹ or AA² comprises, independently in each instance, an amino acid selected from alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is an amino acid selected from alanine, valine, leucine, isoleucine, methionine, tryptophan, phenylalanine, proline, glycine, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is lysine. In some embodiments, AA¹ is lysine or a derivative of lysine. In some embodiments, AA¹ is glutamic acid. In some embodiments, the AA² is valine-citrulline. In some embodiments, the AA² is citrulline-valine. In some embodiments, the AA² is valine-alanine. In some embodiments, the AA² is alanine-valine. In some embodiments, the AA² is valine-glycine. In some embodiments, the AA² is glycine-valine. In some embodiments, the AA¹-AA² is glutamine-valine-citrulline. In some embodiments, the AA¹-AA² is lysine-valine-citrulline. In some embodiments, the AA¹-AA² is lysine-valine-alanine. In some embodiments, the AA¹-AA² is glutamine-valine-alanine. In some embodiments, the lysine is L-lysine. In some embodiments, the lysine is D-lysine.

In some embodiments, the linker is selected from:

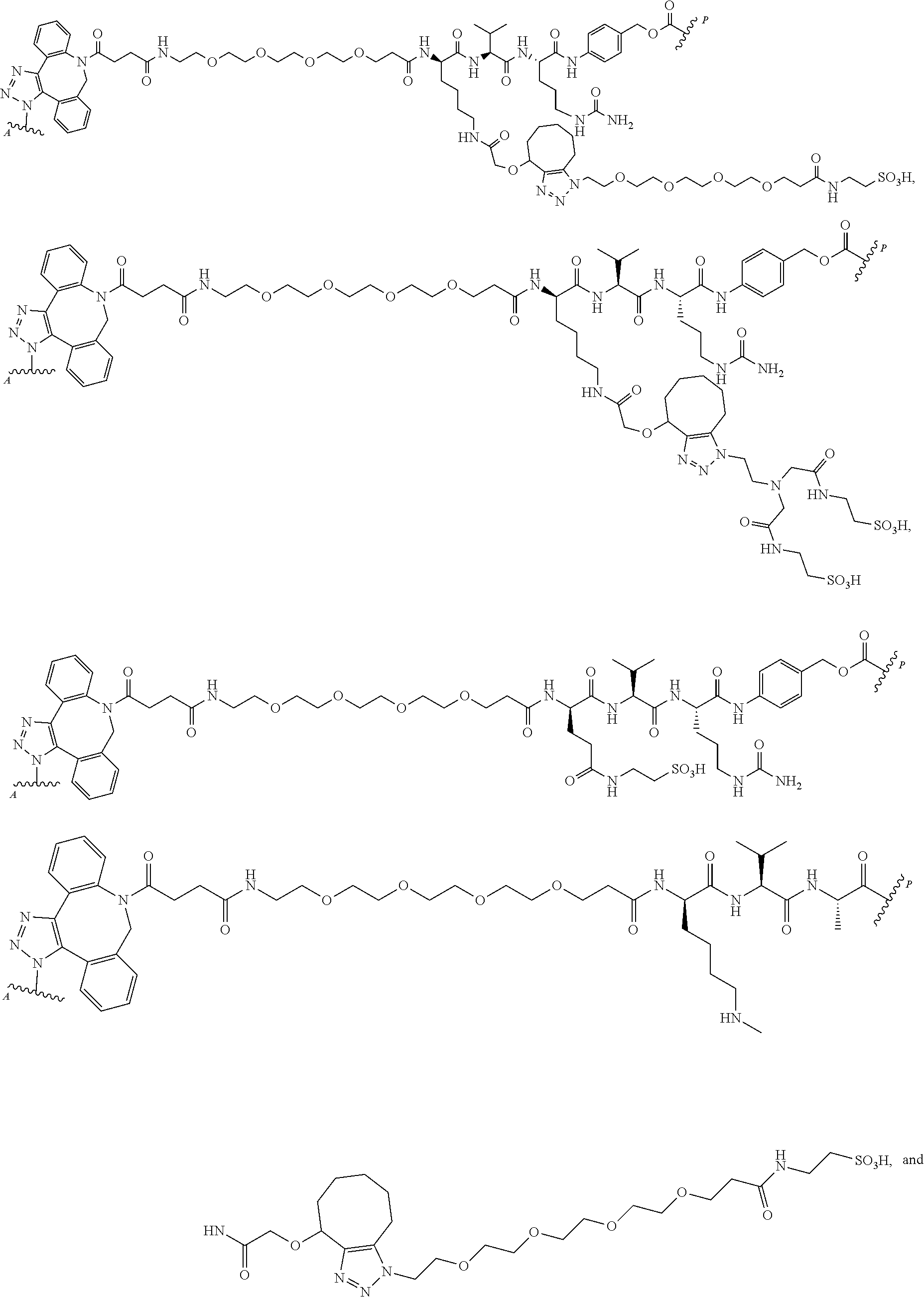

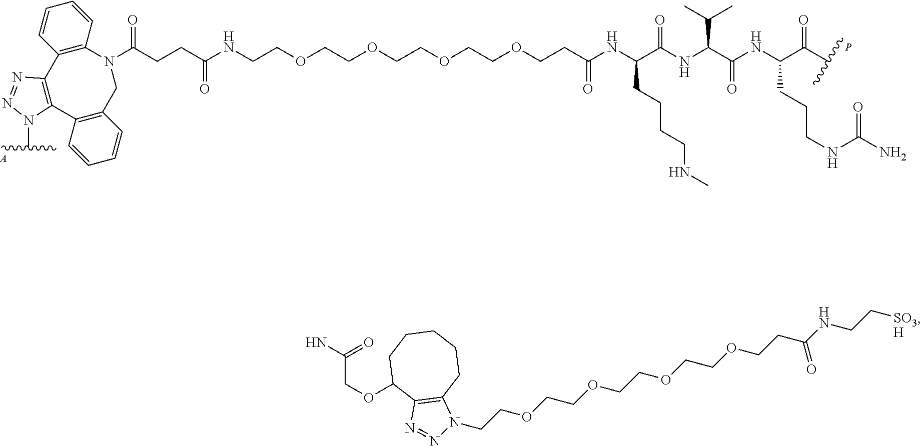

or a stereoisomeric form thereof, or a regioisomer thereof, or a mixture of regioisomers thereof, wherein each is a bond to a binding protein of this disclosure; and each

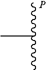

is a bond to the payload residue (i.e., a GR agonist).

8. Category VIII Linkers

Exemplary linkers are described in some detail in, for example: PCT Publication No. 2018/089373, the contents of which is incorporated by reference herein in its entirety.

In some embodiments, the linker comprises the following moiety:

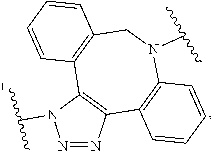 [SMILES: *N1Cc2ccccc2-c2c(nnn2*)-c2ccccc21]

its regioisomer, or mixture thereof, wherein one of the [symbol] shown is a bond to a binding protein of this disclosure.

In some examples, the linker comprises the following

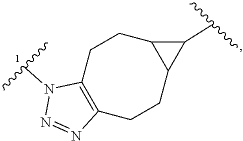 [SMILES: *C1C2CCc3nnn(*)c3CCC12]

its regioisomer, or mixture thereof, wherein one of the [symbol] shown is a bond to a binding protein of this disclosure.

In some examples, the linker comprises the following moiety:

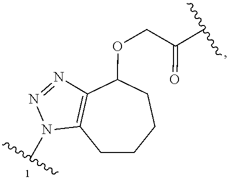 [SMILES: *C(=O)COC1CCCCc2c1nnn2*]

its regioisomer, or mixture thereof, wherein one of the [symbol] shown is a bond to a binding protein of this disclosure).

In some examples, the linker comprises the following moiety:

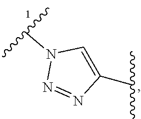 [SMILES: *c1cn(*)nn1]

its regioisomer, or mixture thereof, wherein one of the [symbol] shown is a bond to a binding protein of this disclosure.

In some examples, the linker comprises the following moiety:

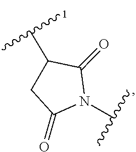 [SMILES: *C1CC(=O)N(*)C1=O]

its regioisomer, or mixture thereof, wherein one of the [symbol] shown is a bond to a binding protein of this disclosure.

In some examples, the linker comprises the following moiety:

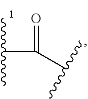 [SMILES: *C(*)=O]

its regioisomer, or mixture thereof, wherein one of the [symbol] shown is a bond to a binding protein of this disclosure.

In some specific embodiments, the linker has the following structure:

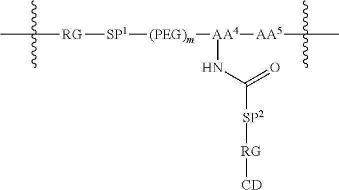

wherein:
RG is a reactive group residue; CD is a cyclodextrin; SP¹ is a spacer group; SP² is a spacer group; AA⁴ is an amino acid residue; AA⁵ is a dipeptide residue; PEG is polyethylene glycol; and m is an integer from 0 to 4.

In some embodiments, SP¹ is absent or a spacer group residue, and wherein SP¹ comprises a trivalent linker; AA⁴ is a trivalent linker comprising an amino acid residue; AA⁵ is a di-peptide residue; PEG is a polyethylene glycol residue; wherein the [symbol] indicates the atom through which the indicated chemical group is bonded to the adjacent groups in the formula, CD is, independently in each instance, absent or a cyclodextrin residue, wherein at least one CD is present, subscript m is an integer from 0 to 5;

In these examples, subscript m is 0, 1, 2, 3, 4, or 5. In some examples, subscript m is 0. In some examples, subscript m is 1. In some examples, subscript m is 2. In some examples, subscript m is 3. In some examples, subscript m is 4. In some examples, subscript m is 5. In some examples, any one of AA⁴ or AA⁵ comprises, independently in each instance, an amino acid selected from alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof. In some embodiments, AA⁴ is an amino acid selected from alanine, valine, leucine, isoleucine, methionine, tryptophan, phenylalanine, proline, glycine, serine, threonine, cysteine, tyrosine, asparagine, glutamine, aspartic acid, glutamic acid, lysine, arginine, histidine, or citrulline, a derivative thereof, or a combination thereof. In some embodiments, AA⁴ is lysine. In some embodiments, AA⁴ is lysine or a derivative of lysine. In some embodiments, the AA⁵ is valine-citrulline. In some embodiments, the AA⁵ is citrulline-valine. In some embodiments, the AA⁵ is valine-alanine. In some embodiments, the AA⁵ is alanine-valine. In some embodiments, the AA⁵ is valine-glycine. In some embodiments, the AA⁵ is glycine-valine. In some embodiments, the AA⁵ glutamate-valine-citrulline. In some embodiments, the AA⁵ is glutamine-valine-citrulline. In some embodiments, the AA⁵ is lysine-valine-alanine. In some embodiments, the AA⁵ is lysine-valine-citrulline. In some embodiments, the AA⁵ is glutamate-valine-citrulline. In some examples, SP¹ is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, and combinations thereof, wherein subscript e is an integer from 0 to 4, subscript u is an integer from 1 to 8, and subscript v is an integer from 1 to 8. In some examples, SP² is independently in each instance, selected from the group consisting of C₁₋₆ alkylene, —NH—, —C(O)—, (—CH₂—CH₂—O)ₑ, —NH—CH₂ —CH₂ —(—O—CH₂ —CH₂)ₑ —C(O)—, —C(O)—(CH₂)ᵤ —C(O)—, —C(O)—NH—(CH₂)ᵥ—, and combinations thereof, wherein subscript e is an integer from 0 to 4, subscript u is an integer from 1 to 8, and subscript v is an integer from 1 to 8.

In some embodiments, the linker is RG^{N}-(SP¹)_{q}-(A)_{z}-. In some embodiments, the linker is —RG^{N}-(SP¹)_{q}-(A)₂-. In some embodiments, the linker is a moiety of Formula (BL^{A1})

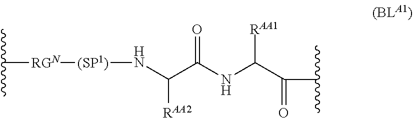

wherein R^{AA1} and R^{AA2} are each, independently, amino acid side chains. In some examples of Formula RL^{A1}, SP¹ is a divalent polyethylene glycol group and RG^{N} is a 1,3-cycloaddition reaction adduct of the reaction between an alkyne and an azide.

In some embodiments, the linker is RG^{N}-(SP¹)_{q}-(A)_{z}-. In some embodiments, the linker is RG^{N}-(SP¹)_{q}-(A)₂-. In some embodiments, the linker is a moiety of Formula (BL^{B1}):

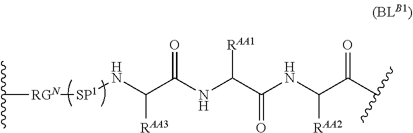

wherein R^{AA1} and R^{AA2} are each, independently, amino acid side chains. R^{AA3} is an amino acid side chain that is bonded directly or indirectly to a cyclodextrin moiety. In some examples of Formula BL^{B1}, SP¹ is a divalent polyethylene glycol group and RG^{N} is a 1,3-cycloaddition reaction adduct of the reaction between an alkyne and an azide.

In some embodiments, the linker has the following structure:

RG^{N}-(SP¹)q-Z¹-Z²—Z³₀₋₁—
wherein:
RG^{N}, SP¹, are as defined herein; q is 0 or 1; Z¹ is a polyethylene glycol or caproyl group; Z² is a dipeptide or tripeptide; and Z³ is a PAB group.

In some embodiments, RG^{N} is derived from a click-chemistry reactive group and Z¹ is a polyethylene glycol group. In some embodiments, RGN—(SP¹)_{q}—Z¹— is:

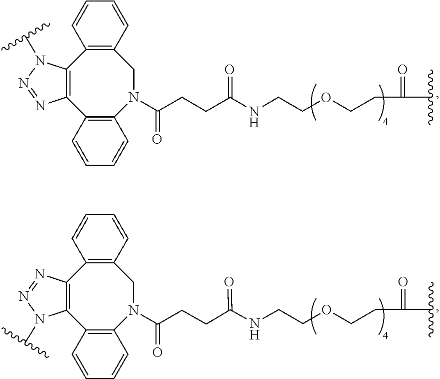

or mixture thereof, or

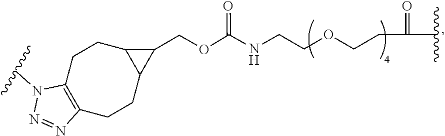

In some embodiments, the dipeptide is valine-citrulline or valine alanine.

In some examples, herein RG^{N} is derived from a click-chemistry reactive group. In some examples, RG^{N} is:

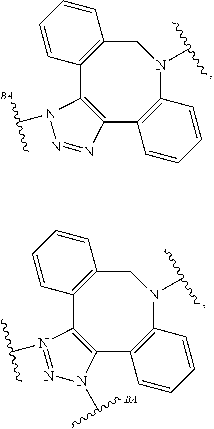

or mixture thereof,

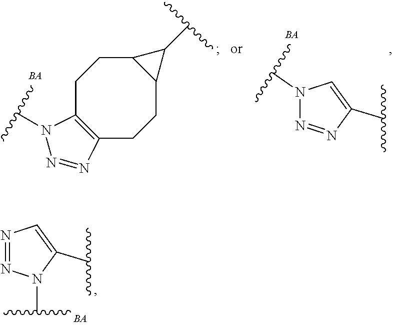 [SMILES: *C1C2CCc3nnn(*)c3CCC12 ; *c1cn(*)nn1 ; *c1cnnn1*]

or mixture thereof, wherein [symbol] denotes bonding to a binding agent (e.g., an antibody or binding fragment thereof).

In some other examples, herein RG^{N} is selected from a group which reacts with a cysteine or lysine residue on an antibody or an antigen-binding fragment thereof. In some examples, RG^{N} is

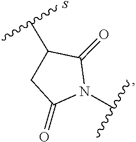

wherein [symbol] is a bond to cysteine of a binding agent, e.g., antibody. In some examples, RG^{N} is

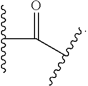 [SMILES: *C(*)=O]

In some embodiments, SP¹ is selected from:

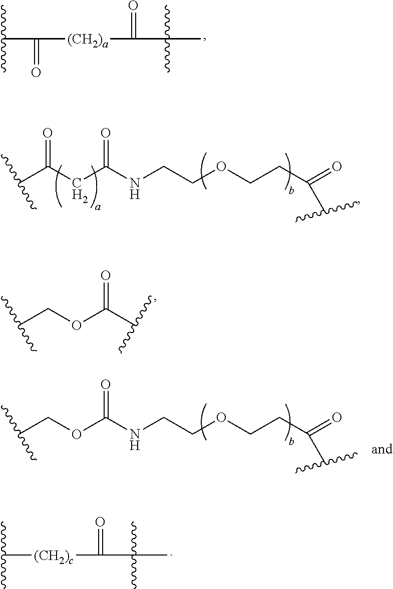

In some examples, SP¹ is

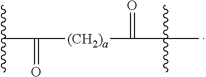

In some other examples, SP¹ is

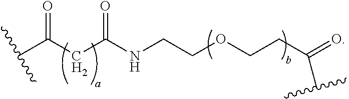

In other examples, SP¹ is

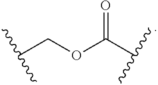 [SMILES: *COC(*)=O]

In still other examples, SP¹ is

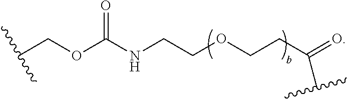

In some other examples, SP¹ is

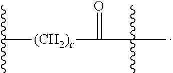

In any of the above examples, subscripts a, b, and c are independently, in each instance, an integer from 1 to 20.

In some embodiments, R^{AA3} is selected from

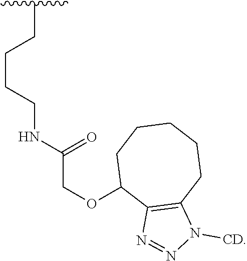

wherein CD is a cyclodextrin moiety. In some embodiments, R^{AA3} is selected from

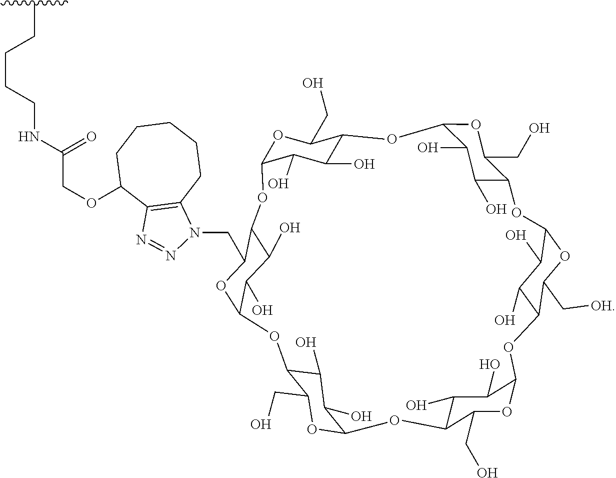 [SMILES: *CCCCNC(=O)COC1CCCCCc2c1nnn2CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

In some enbodiments, SP¹ is selected from:

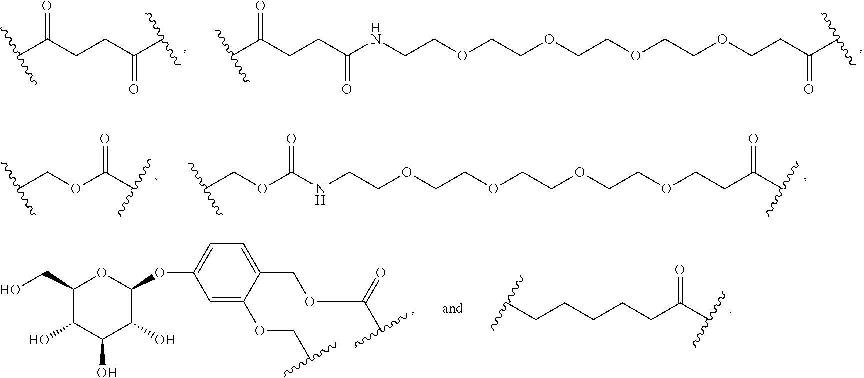 [SMILES: *C(=O)CCC(*)=O ; *C(=O)CCOCCOCCOCCOCCNC(=O)CCC(*)=O ; *COC(*)=O ; *COC(=O)NCCOCCOCCOCCOCCC(*)=O ; *COc1cc(O[C@@H]2O[C@H](CO)[C@@H](O)[C@H](O)[C@H]2O)ccc1COC(*)=O ; *CCCCCC(*)=O]

In some examples, SP¹ is

In some examples, SP¹ is

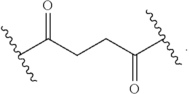 [SMILES: *C(=O)CCC(*)=O]

In some examples, SP¹ is

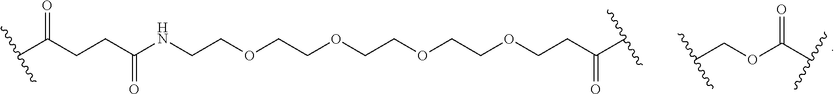 [SMILES: *C(=O)CCOCCOCCOCCOCCNC(=O)CCC(*)=O ; *COC(*)=O]

In some examples, SP¹ is

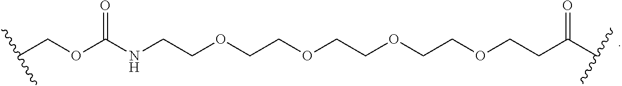 [SMILES: *COC(=O)NCCOCCOCCOCCOCCC(*)=O]

In some examples, SP¹ is

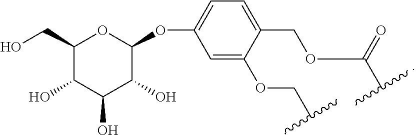 [SMILES: *COc1cc(O[C@@H]2O[C@H](CO)[C@@H](O)[C@H](O)[C@H]2O)ccc1COC(*)=O]

In some examples, SP¹ is

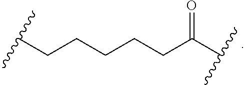 [SMILES: *CCCCCC(*)=O]

In some examples, SP¹ is

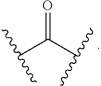 [SMILES: *C(*)=O]

In some examples, SP¹ is

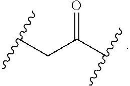 [SMILES: *CC(*)=O]

In some examples, SP¹ is

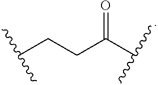 [SMILES: *CCC(*)=O]

In some examples, SP¹ is

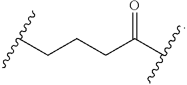 [SMILES: *CCCC(*)=O]

In some examples, SP¹ is

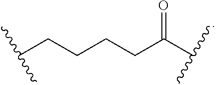 [SMILES: *CCCCC(*)=O]

In some examples, SP¹ is

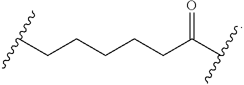 [SMILES: *CCCCCC(*)=O]

In some embodiments, the linker comprises:

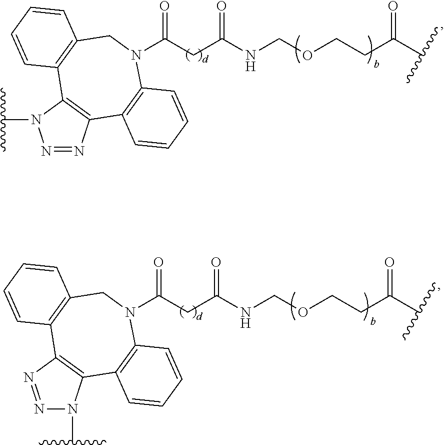

or mixture thereof,

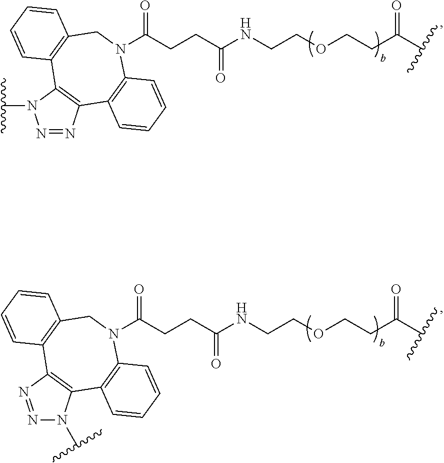

or mixture thereof,

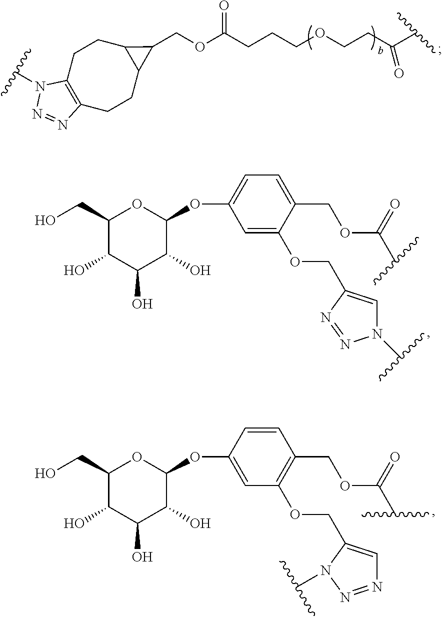

or mixture thereof, or

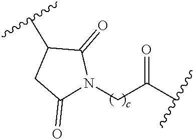

In some of these examples, subscripts b, c, and d are independently, in each instance, an integer from 1 to 20.

In some embodiments, the linker comprises:

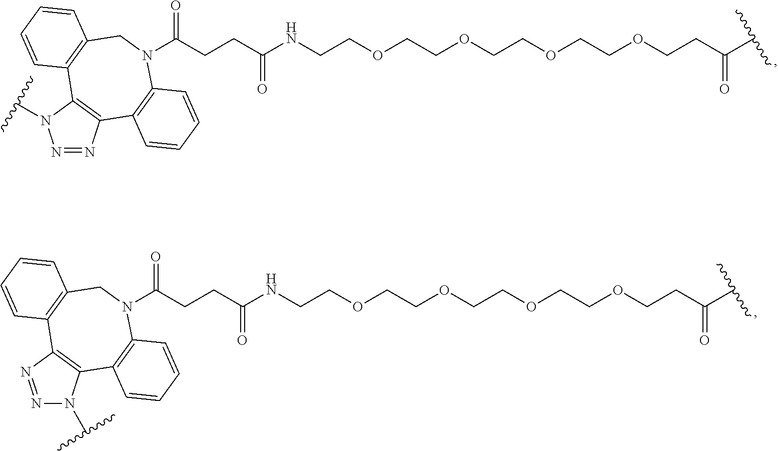 [SMILES: *C(=O)CCOCCOCCOCCOCCNC(=O)CCC(=O)N1Cc2ccccc2-c2c(nnn2*)-c2ccccc21 ; *C(=O)CCOCCOCCOCCOCCNC(=O)CCC(=O)N1Cc2ccccc2-c2nnn(*)c2-c2ccccc21]

or mixture thereof,

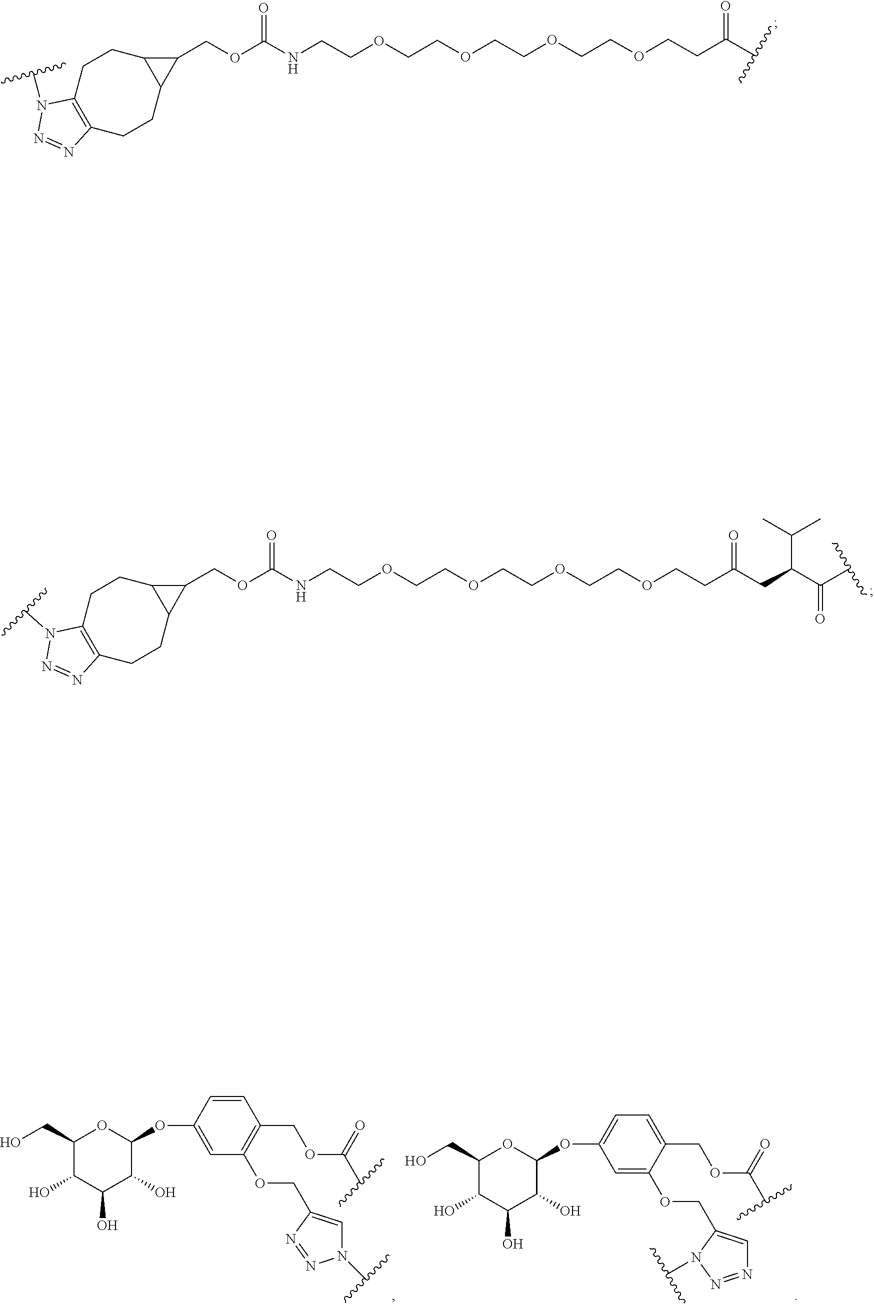 [SMILES: *C(=O)CCOCCOCCOCCOCCNC(=O)OCC1C2CCc3nnn(*)c3CCC21 ; *C(=O)[C@@H](CC(=O)CCOCCOCCOCCOCCNC(=O)OCC1C2CCc3nnn(*)c3CCC21)C(C)C ; *C(=O)OCc1ccc(O[C@@H]2O[C@H](CO)[C@@H](O)[C@H](O)[C@H]2O)cc1OCc1cn(*)nn1 ; *C(=O)OCc1ccc(O[C@@H]2O[C@H](CO)[C@@H](O)[C@H](O)[C@H]2O)cc1OCc1cnnn1*]

or mixture thereof, or

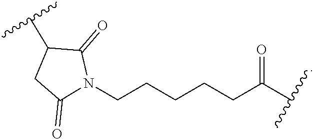 [SMILES: *C(=O)CCCCCN1C(=O)CC(*)C1=O]

In some embodiments, A is a peptide selected from valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some examples, A is valine-citrulline or citrulline-valine. In some examples, A is valine-alanine or alanine-valine. In some examples, A is lysine-valine-alanine or alanine-valine-lysine.

In some examples, A is lysine-valine-citrulline or citrulline-valine-lysine. In some examples, A is valine. In some examples, A is alanine. In some examples, A is citrulline.

In some examples, A is:

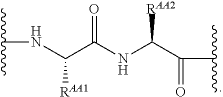

In some of these examples, R^{AA1} is an amino acid side chain, and wherein R^{AA2} is an amino acid side chain. In some examples, A is:

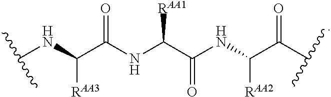

In some of these examples, R^{AA1} is an amino acid side chain, R^{AA2} is an amino acid side chain, and R^{AA3} is an amino acid side chain that is bonded directly or indirectly to a cyclodextrin moiety. In some examples, A is:

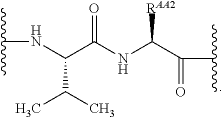

In some examples, A is:

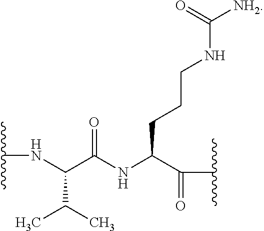 [SMILES: *N[C@H](C(=O)N[C@@H](CCCNC(N)=O)C(*)=O)C(C)C]

In some examples, A is:

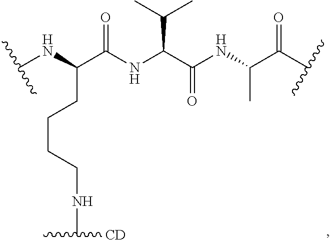

wherein [symbol] represents a direct or indirect bond to a cyclodextrin moiety.

In some examples, including any of the foregoing, CD is, independently in each instance, selected from

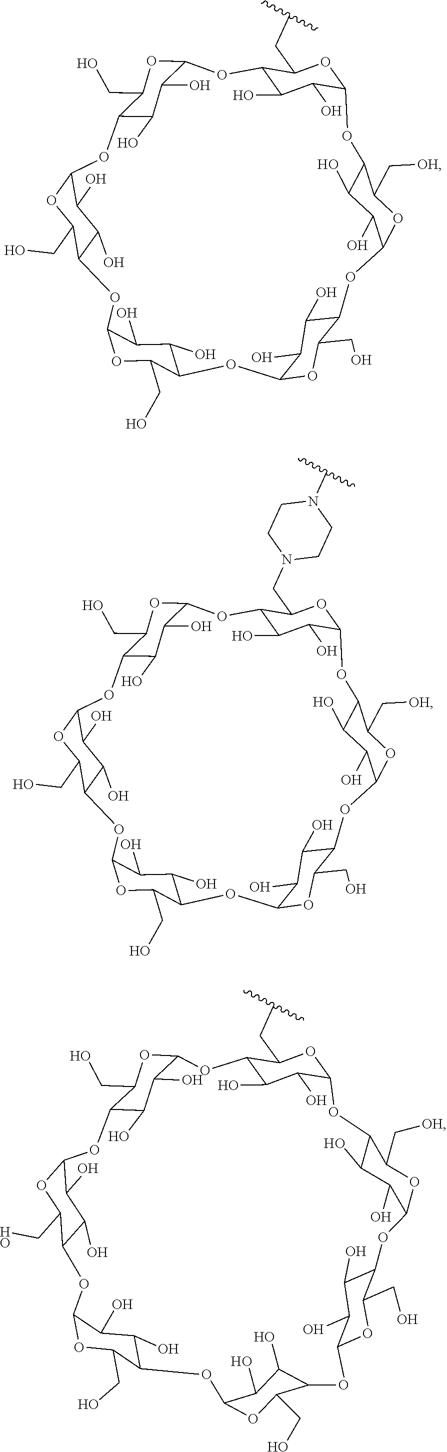 [SMILES: *CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O ; *N1CCN(CC2OC3OC4C(CO)OC(OC5C(CO)OC(OC6C(CO)OC(OC7C(CO)OC(OC8C(CO)OC(OC2C(O)C3O)C(O)C8O)C(O)C7O)C(O)C6O)C(O)C5O)C(O)C4O)CC1 ; *CC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

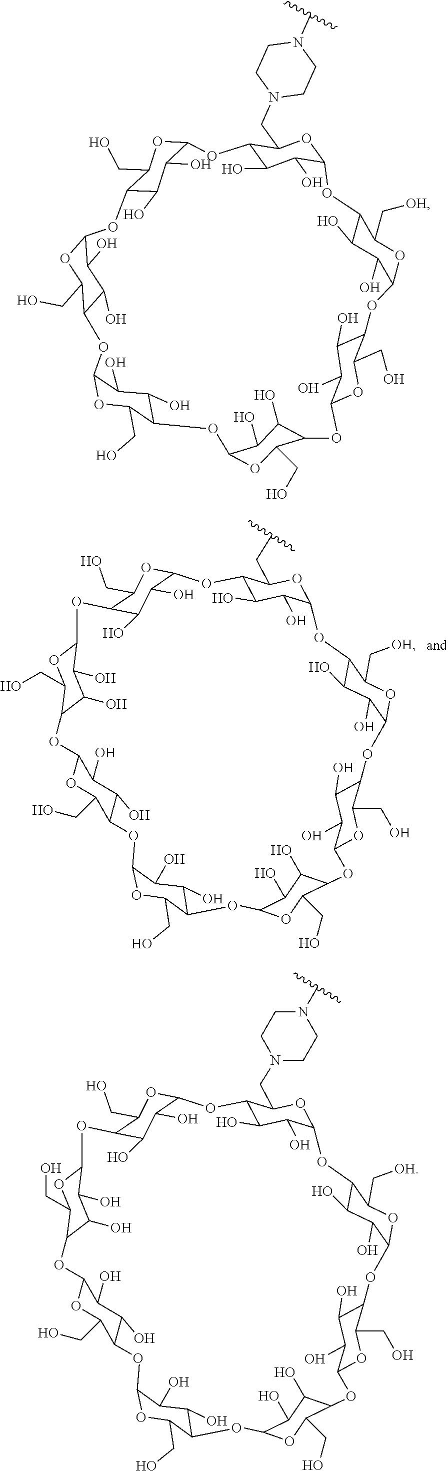 [SMILES: *N1CCN(CC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)CC1 ; *CC1OC(OC2C(CO)OC(OC3C(CO)OC(OC4C(CO)OC(OC5C(CO)OC(C)C(O)C5O)C(O)C4O)C(O)C3O)C(O)C2O)C(O)C(O)C1OC1OC(CO)C(OC2OC(CO)C(OC3OC(CO)C(O)C(O)C3O)C(O)C2O)C(O)C1O ; "and" ; *N1CCN(CC2OC(OC3C(CO)OC(OC4C(CO)OC(OC5C(CO)OC(OC6C(CO)OC(C)C(O)C6O)C(O)C5O)C(O)C4O)C(O)C3O)C(O)C(O)C2OC2OC(CO)C(OC3OC(CO)C(OC4OC(CO)C(O)C(O)C4O)C(O)C3O)C(O)C2O)CC1]

In some examples, the CD is

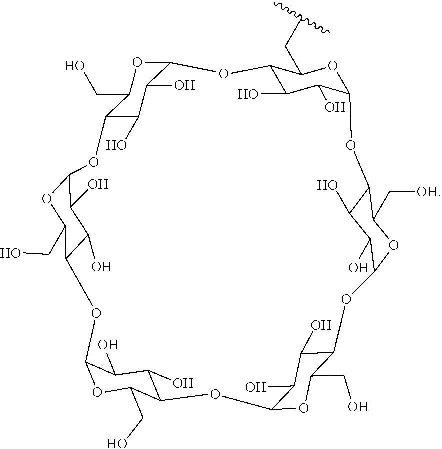 [SMILES: *CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

In some examples, the CD is

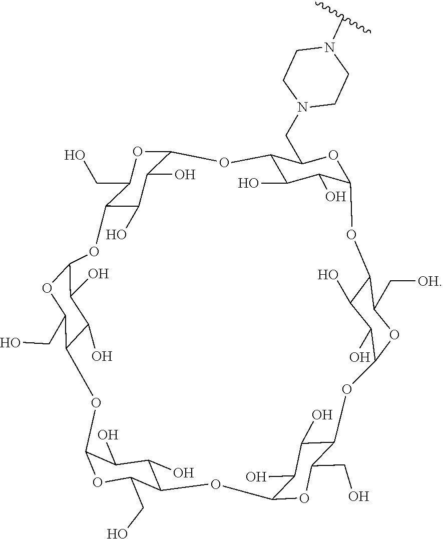 [SMILES: *N1CCN(CC2OC3OC4C(CO)OC(OC5C(CO)OC(OC6C(CO)OC(OC7C(CO)OC(OC8C(CO)OC(OC2C(O)C3O)C(O)C8O)C(O)C7O)C(O)C6O)C(O)C5O)C(O)C4O)CC1]

In some examples, the CD is

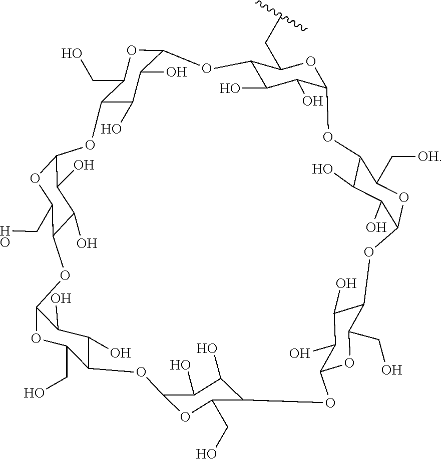 [SMILES: *CC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

In some examples, the CD is

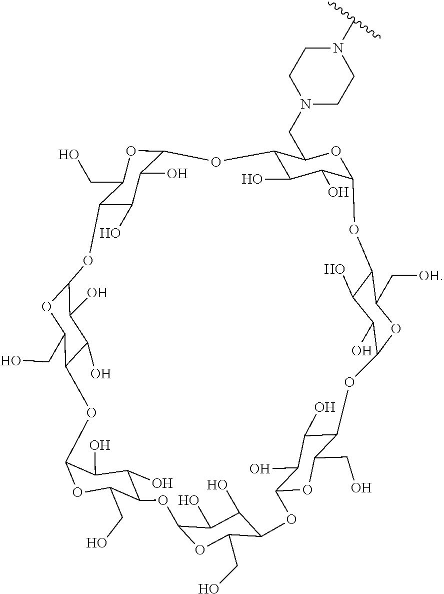 [SMILES: *N1CCN(CC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)CC1]

In some examples, the CD is

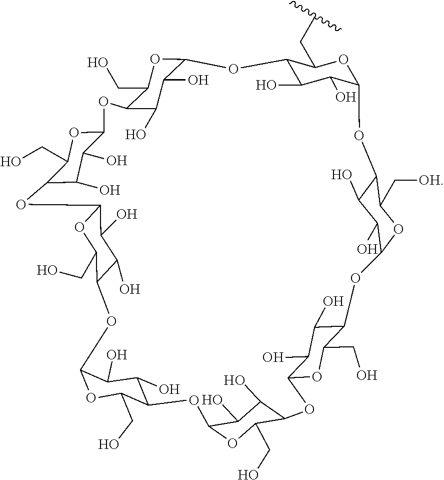 [SMILES: *CC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O]

In some examples, the CD is

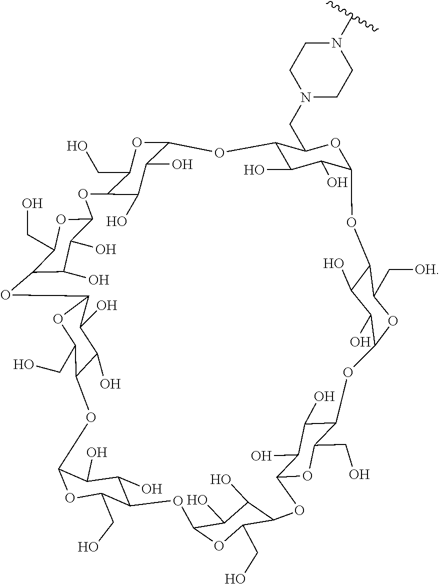 [SMILES: *N1CCN(CC2OC3OC4C(CO)OC(OC5C(CO)OC(OC6C(CO)OC(OC7C(CO)OC(OC8C(CO)OC(OC9C(CO)OC(OC%10C(CO)OC(OC2C(O)C3O)C(O)C%10O)C(O)C9O)C(O)C8O)C(O)C7O)C(O)C6O)C(O)C5O)C(O)C4O)CC1]

In some examples, A is

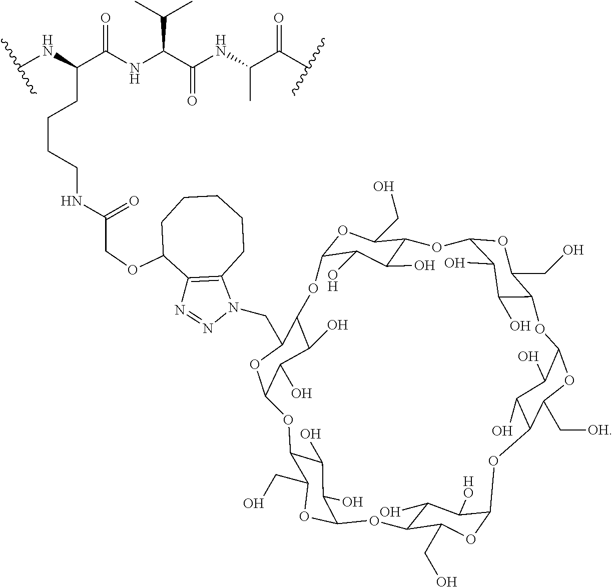 [SMILES: *N[C@H](CCCCNC(=O)COC1CCCCCc2c1nnn2CC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O)C(=O)N[C@H](C(=O)N[C@@H](C)C(*)=O)C(C)C]

In some examples, R^{a} is H. In some examples, R^{a} is alkyl. In some examples, R^{a} is methyl, ethyl, n-propyl, i-propyl, n-butyl, t-butyl, i-butyl, or pentyl.

In some embodiments, B is aryl. In some examples, B is phenyl. In some embodiments, B is phenyl or pyridinyl. In some examples herein, B is:

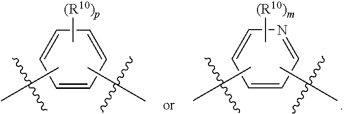

In these examples, R¹⁰ is alkyl, alkenyl, alkynyl, alkoxy, aryl, alkylaryl, arylalkyl, halo, haloalkyl, haloalkoxy, heteroaryl, heterocycloalkyl, hydroxyl, cyano, nitro,

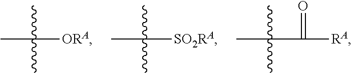

NR^{a}R^{b}, or azido. In these examples, subscripts p and m are independently, in each instance, selected from an integer from 0 to 4.

In some examples herein, B is

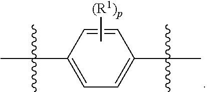

In these examples, p is 0, 1, 2, 3 or 4. In some of these examples, R¹ is, independently at each occurrence, alkyl, alkoxy, haloalkyl, or halo. In some examples, R¹ is alkyl. In some examples, R¹ is alkoxy. In some examples, R¹ is haloalkyl. In some examples, R¹ is halo.

In some embodiments, —(NR^{a})^{s}—(B)ₜ—(CH₂)ᵤ—(O)ᵥ—(SP²)_{w} is:

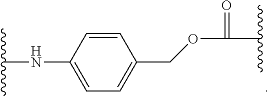 [SMILES: *Nc1ccc(COC(*)=O)cc1]

The binding agent linkers can be bonded to the binding agent, e.g., antibody or antigen-binding molecule, through an attachment at a particular amino acid within the antibody or antigen-binding molecule. Exemplary amino acid attachments that can be used in the context of this aspect of the disclosure include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can be conjugated via glutamine via transglutaminase-based chemoenzymatic conjugation (see, e.g., Dennler et al., Bioconjugate Chem. 2014, 25, 569-578). Linkers can also be conjugated to an antigen binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, WO 2014/197854, and Shaunak et al., Nat. Chem. Biol., 2006, 2:312-313). In some examples, the binding agent is an antibody, and the antibody is bonded to the linker through a lysine residue. In some embodiments, the antibody is bonded to the linker through a cysteine residue.

9. Category IX Linkers

Exemplary linkers are described in some detail in, for example: PCT Publication No. WO 2019/094395 and U.S. Pat. No. 10,711,032, the linkers of each of which are incorporated by reference herein in their entirety.

In some specific embodiments, the linker has the following structure:

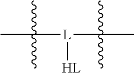

wherein L is a trivalent linker; and HL is a hydrophilic residue.

In some embodiments, the linker has the following structure:

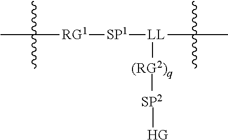

wherein: LL is a trivalent linker; RG¹ and RG² are reactive group residues; SP¹ and SP² are independently, in each instance, absent, or a spacer group residue; HG is a hydrophilic residue; and q is 0 or 1.

In some instances, HG is a terminal hydrophilic group. In some instances, HG comprises one terminal sulfonic acid group (SO₃H), or salts thereof. In other instances, HG comprises more than one terminal sulfonic acid groups, or salts thereof. In some instances, HG comprises one terminal taurine group or salts thereof. In other instances, HG comprises more than one terminal taurine groups or salts thereof. In some instances, HG comprises one terminal phosphonic acid group (PO₃H), or salt thereof. In other instances, HG comprises more than one terminal phosphonic acid groups, or salts thereof. In some instances, HG comprises one terminal amine group, or salt thereof. In other instances, HG comprises more than one terminal amine group, or salts thereof. In further instances, HG comprises one terminal quaternary amine group, or salts thereof. In further instances, HG comprises more than one terminal quaternary amine group, or salts thereof. In some instances, HG comprises one terminal sugar group, or salt thereof. In other instances, HG comprises more than one terminal sugar groups, or salts thereof.

In some embodiments, the linker has the following structure:

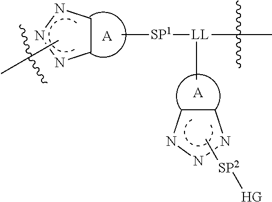

wherein ring A is fused to the triazole and is selected from the group consisting of cycloalkyl, cycloalkenyl, heterocycloalkyl, and heterocycloalkenyl; wherein cycloalkyl, cycloalkenyl, heterocycloalkyl, and heterocycloalkenyl are optionally substituted with alkyl, —OH, or —NR^{a}R^{b}, where each of R^{a} and R^{b} is alkyl or H.

In some embodiments, the linker has the following structure:

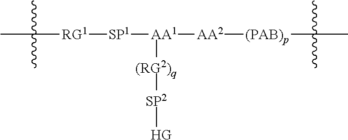

wherein RG¹, SP¹, RG², SP² and HG are as defined above, AA¹ is a trivalent linker comprising an amino acid residue and is directly or indirectly linked to a binding protein of this disclosure, a payload and a hydrophilic group; AA² is a dipeptide, tripeptide or tetrapeptide residue; and PAB is

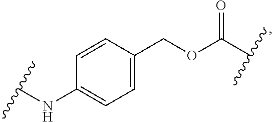 [SMILES: *Nc1ccc(COC(*)=O)cc1]

wherein the

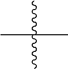

indicates the atom through which the PAB is bonded to the adjacent groups in the formula; subscript p is 0 or 1; and subscript q is 0 or 1. In some instances, subscript p is 0 and subscript q is 0. In some instances, subscript p is 1; and subscript q is 0. In some instances, subscript p is 0; and subscript q is 1. In some instances, subscript p is 1; and subscript q is 1. In some instances SP¹ comprises from 0-5 polyethylene glycol (PEG) residues. In some instances SP² comprises from 0-5 PEG residues. In some examples, SP¹ is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, polyglycine (e.g., ((glycine)₄-serine)_{f} wherein subscript f is an integer from 1 to 6), and combinations thereof, wherein subscript e is an integer from 0 to 4, subscript u is an integer from 1 to 8, and subscript v is an integer from 1 to 8. In some examples, SP² is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ— C(O)—, —C(O)—NH—(CH₂)ᵥ—, polyglycine (e.g., ((glycine)₄-serine)f wherein subscript f is an integer from 1 to 6), and combinations thereof, wherein subscript e is an integer from 0 to 4, subscript u is an integer from 1 to 8, and subscript v is an integer from 1 to 8. In some examples, any one of AA¹ or AA² comprises, independently in each instance, an amino acid selected from alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof.

In some embodiments, AA¹ is an amino acid selected from alanine, valine, leucine, isoleucine, methionine, tryptophan, phenylalanine, proline, glycine, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is an amino acid with three functional groups to link to a payload, to a binding agent (e.g., antibody or antigen binding fragment thereof), and to a linker comprising a hydrophilic group, e.g., lysine, asparagine, glutamic acid, aspartic acid, glutamine, cysteine, threonine, serine, or tyrosine. In some embodiments, AA¹ is lysine. In some embodiments, AA¹ is lysine or a derivative of lysine. In some embodiments, AA¹ is L-lysine. In some embodiments, the AA¹ is D-lysine. In some embodiments, AA¹ is glutamine. In some embodiments, AA¹ is glutamic acid. In some embodiments, AA¹ is aspartic acid. In some embodiments, the AA² is valine-citrulline. In some embodiments, the AA² is citrulline-valine. In some embodiments, the AA² is valine-alanine. In some embodiments, the AA² is alanine-valine. In some embodiments, the AA² is valine-glycine. In some embodiments, the AA² is glycine-valine. In some embodiments, the AA¹-AA² is glutamine-valine-citrulline. In some embodiments, the AA¹-AA² is lysine-valine-citrulline. In some embodiments, the AA¹-AA² is lysine-valine-alanine. In some embodiments, the AA¹-AA² is glutamine-valine-alanine. In some embodiments, ((glycine)₄-serine)_{f} is (glycine)₄-serine.

In some more specific examples, the linker has one of the following structures

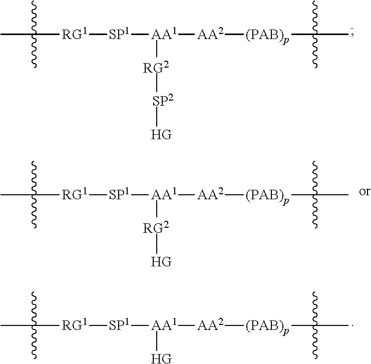

AA¹ is a trivalent linker comprising an amino acid residue and is directly or indirectly linked to an antibody, a payload and a hydrophilic group; AA² is a dipeptide, tripeptide, or tetrapeptide residue; and PAB is

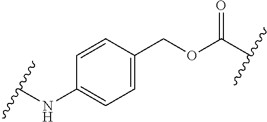 [SMILES: *Nc1ccc(COC(*)=O)cc1]

wherein the

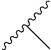

indicates the atom through which the PAB is bonded to the adjacent groups in the formula. In some instances, subscript p is 0. In some instances, subscript p is 1. In some instances, SP¹ comprises from 0-5 polyethylene glycol (PEG) residues. In some instances, SP² comprises from 0-5 PEG residues. In some examples, SP¹ is independently in each instance, selected from the group consisting of C₁₋₆ alkylene, —NH—, —C(O)—(—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, polyglycine (e.g., ((glycine)₄-serine)_{f} wherein subscript f is an integer from 1 to 6), and combinations thereof, wherein subscript e is an integer from 0 to 4, subscript u is an integer from 1 to 8, and subscript v is an integer from 1 to 8. In some examples, SP² is independently in each instance, selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, polyglycine (e.g., ((glycine)₄-serine)_{f} wherein subscript f is an integer from 1 to 6), and combinations thereof, wherein subscript e is an integer from 0 to 4, subscript u is an integer from 1 to 8, and subscript v is an integer from 1 to 8. In some examples, any one of AA¹ or AA² comprises, independently in each instance, an amino acid selected from alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is an amino acid selected from alanine, valine, leucine, isoleucine, methionine, tryptophan, phenylalanine, proline, glycine, serine, threonine, cysteine, tyrosine, asparagine, glutamine, aspartic acid, glutamic acid, lysine, arginine, histidine, or citrulline, a derivative thereof, or a combination thereof. In some embodiments, AA¹ is lysine. In some embodiments, AA¹ is an amino acid with three functional groups to link to a payload, to a binding agent (e.g., antibody or antigen binding fragment thereof), and to a linker comprising a hydrophilic group, e.g., lysine, asparagine, glutamic acid, aspartic acid, glutamine, cysteine, threonine, serine, or tyrosine. In some embodiments, AA¹ is lysine or a derivative of lysine. In some embodiments, AA¹ is L-lysine. In some embodiments, the AA¹ is D-lysine. In some embodiments, AA¹ is glutamine. In some embodiments, AA¹ is glutamic acid. In some embodiments, AA¹ is aspartic acid. In some embodiments, the AA¹ is valine-citrulline. In some embodiments, the AA¹ is citrulline-valine. In some embodiments, the AA¹ is valine-alanine. In some embodiments, the AA¹ is alanine-valine. In some embodiments, the AA¹ is valine-glycine. In some embodiments, the AA¹ is glycine-valine. In some embodiments, the AA¹-AA² is glutamine-valine-citrulline. In some embodiments, the AA¹-AA² is lysine-valine-citrulline. In some embodiments, the AA¹-AA² is lysine-valine-alanine. In some embodiments, the AA¹-AA² is glutamine-valine-alanine. In some embodiments, ((glycine)₄-serine)_{f} is (glycine)₄-serine.

In some embodiments, the linker has one of the following structures:

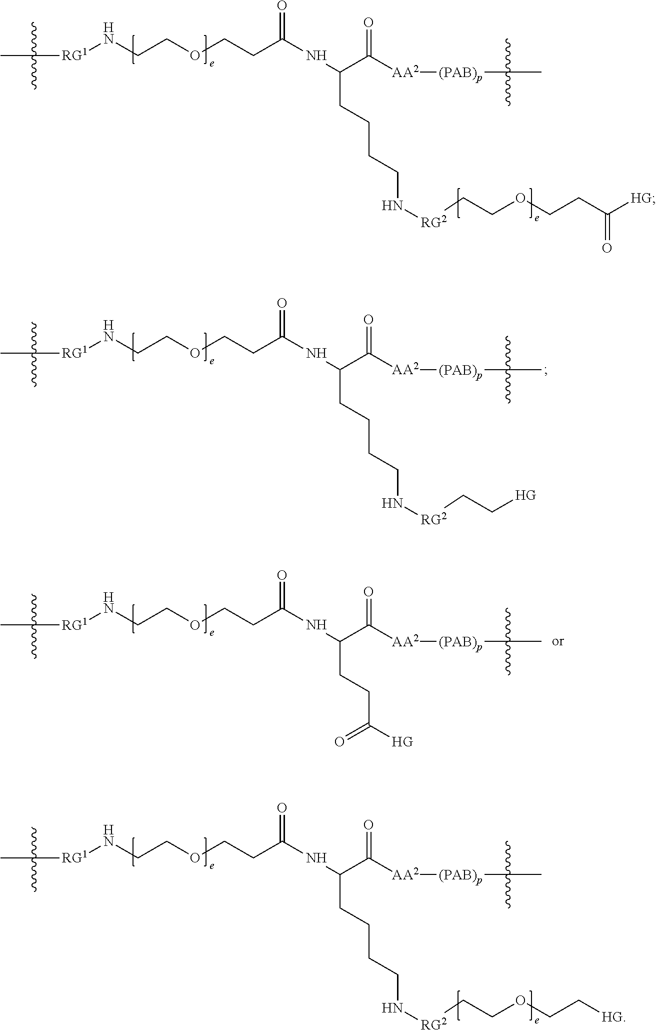

RG¹ and RG² are independently in each instance, a click chemistry residue. In some examples, RG¹ and RG² independently in each instance, comprise a triazaole or a fused triazole. In some instances, RG¹ and RG² are independently, in each instance, selected from the group consisting of:

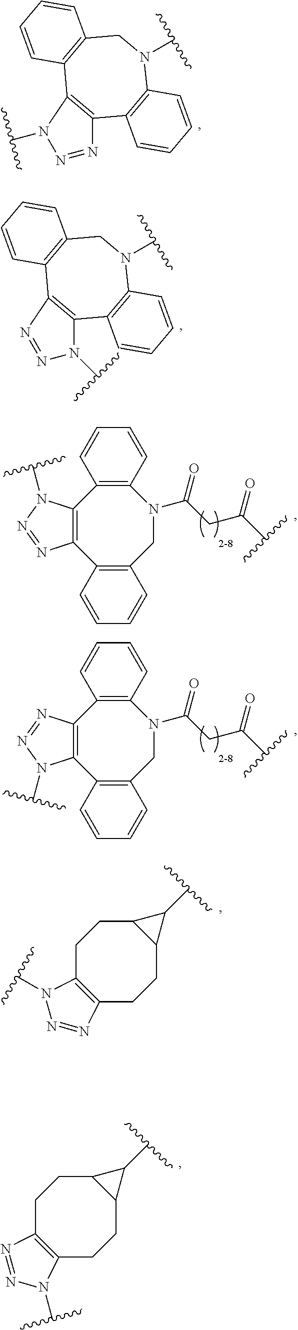

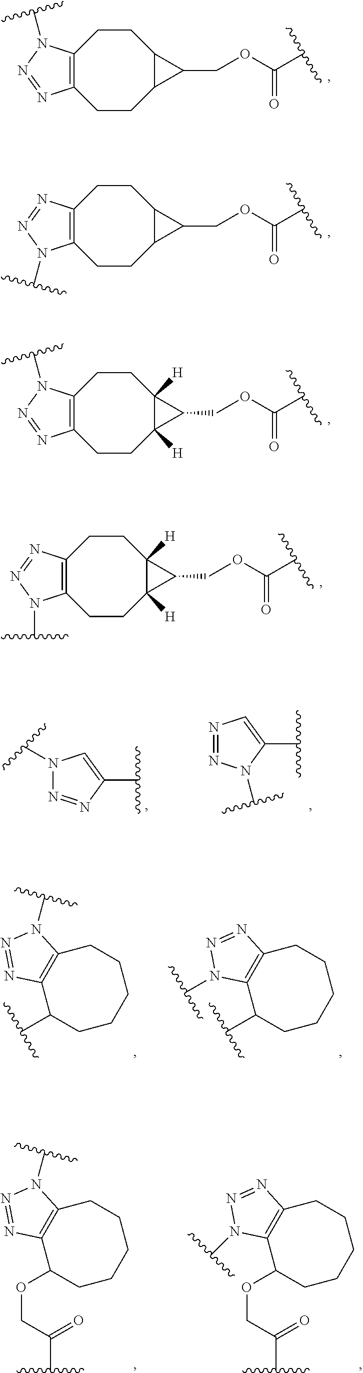

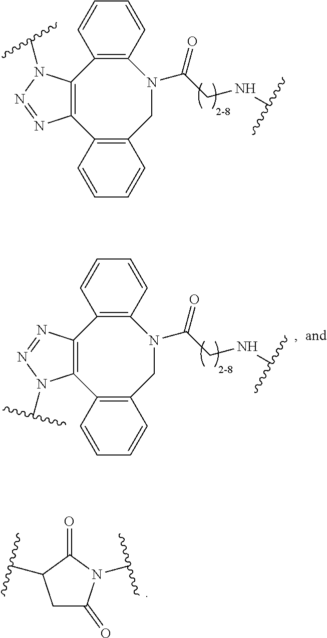

wherein the

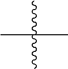

indicates the atom through which the RG¹ or RG² is bonded to the adjacent groups in the formula.

In some embodiments,

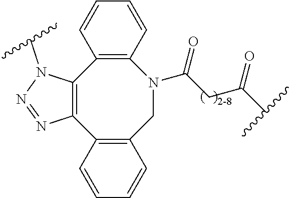

is

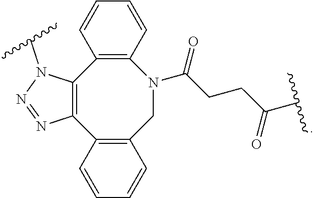 [SMILES: *C(=O)CCC(=O)N1Cc2ccccc2-c2nnn(*)c2-c2ccccc21]

In some embodiments,

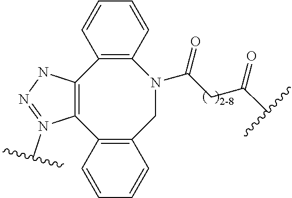

is

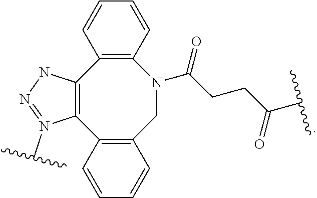

In some embodiments,

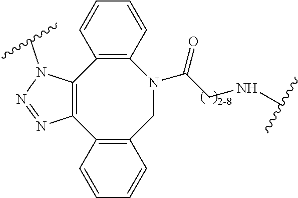

is

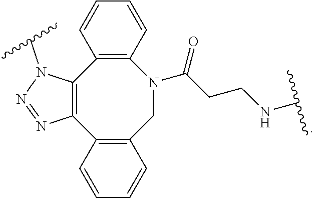 [SMILES: *NCCC(=O)N1Cc2ccccc2-c2nnn(*)c2-c2ccccc21]

In some embodiments,

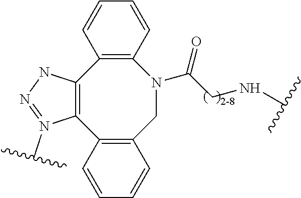

is

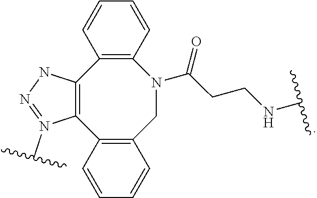

In certain instances, RG¹ and RG² are independently, in each instance, as shown in Table 5 below.

TABLE 5
RG1 and RG2

| RG¹ | RG² |
|---|---|
| 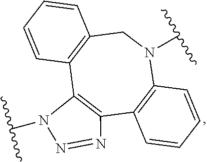 [SMILES: *N1Cc2ccccc2-c2c(nnn2*)-c2ccccc21] | 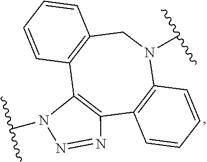 [SMILES: *N1Cc2ccccc2-c2c(nnn2*)-c2ccccc21] |
| or | or |
| 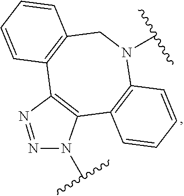 [SMILES: *N1Cc2ccccc2-c2nnn(*)c2-c2ccccc21] | 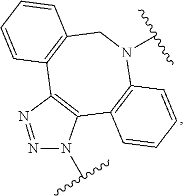 [SMILES: *N1Cc2ccccc2-c2nnn(*)c2-c2ccccc21] |
| 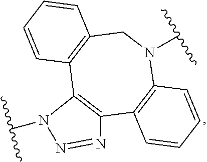 [SMILES: *N1Cc2ccccc2-c2c(nnn2*)-c2ccccc21] | 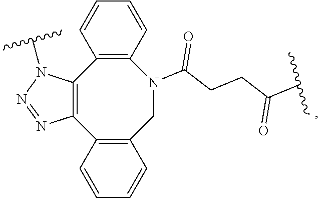 [SMILES: *C(=O)CCC(=O)N1Cc2ccccc2-c2nnn(*)c2-c2ccccc21] |
| or | or |
| 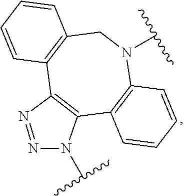 [SMILES: *N1Cc2ccccc2-c2nnn(*)c2-c2ccccc21] | 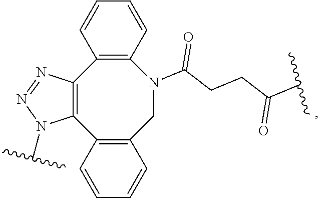 [SMILES: *C(=O)CCC(=O)N1Cc2ccccc2-c2c(nnn2*)-c2ccccc21] |
| 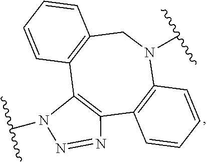 [SMILES: *N1Cc2ccccc2-c2c(nnn2*)-c2ccccc21] | 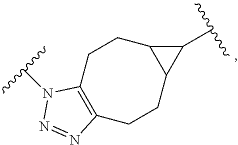 [SMILES: *C1C2CCc3nnn(*)c3CCC12] |
| or | |
| 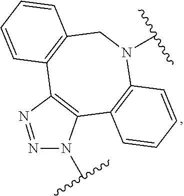 [SMILES: *N1Cc2ccccc2-c2nnn(*)c2-c2ccccc21] | 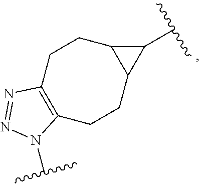 [SMILES: *C1C2CCc3nnn(*)c3CCC12] |
| 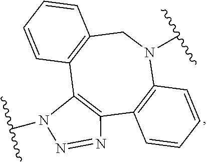 [SMILES: *N1Cc2ccccc2-c2c(nnn2*)-c2ccccc21] | 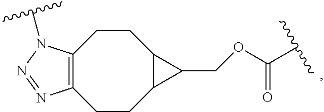 [SMILES: *C(=O)OCC1C2CCc3nnn(*)c3CCC21] |
| or | |
| 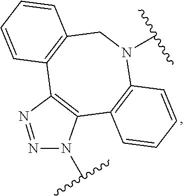 [SMILES: *N1Cc2ccccc2-c2nnn(*)c2-c2ccccc21] | 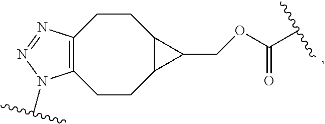 [SMILES: *C(=O)OCC1C2CCc3nnn(*)c3CCC21] |
| | 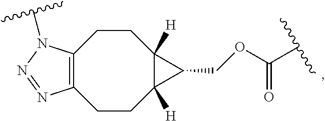 [SMILES: *C(=O)OC[C@@H]1[C@@H]2CCc3nnn(*)c3CC[C@@H]21] |
| | or |
| | 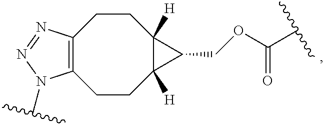 [SMILES: *C(=O)OC[C@@H]1[C@@H]2CCc3c(nnn3*)CC[C@@H]21] |
| 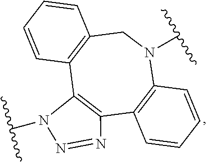 [SMILES: *N1Cc2ccccc2-c2c(nnn2*)-c2ccccc21] | 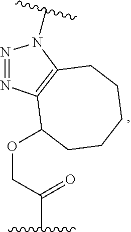 [SMILES: *C(=O)COC1CCCCCc2c1nnn2*] |
| or | or |
| 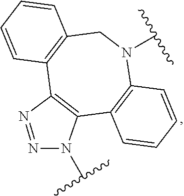 [SMILES: *N1Cc2ccccc2-c2nnn(*)c2-c2ccccc21] | 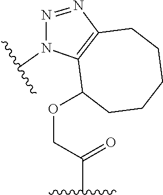 [SMILES: *C(=O)COC1CCCCCc2nnn(*)c21] |
| 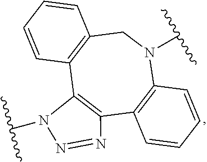 [SMILES: *N1Cc2ccccc2-c2c(nnn2*)-c2ccccc21] | 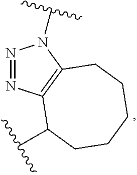 [SMILES: *C1CCCCCc2c1nnn2*] |
| or | or |
| 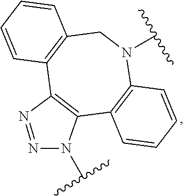 [SMILES: *N1Cc2ccccc2-c2nnn(*)c2-c2ccccc21] | 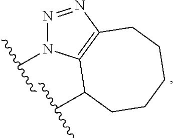 [SMILES: *C1CCCCCc2nnn(*)c21] |
| 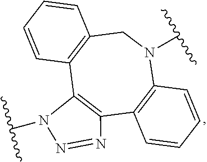 [SMILES: *N1Cc2ccccc2-c2c(nnn2*)-c2ccccc21] | 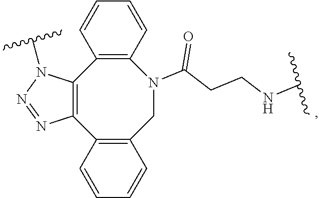 [SMILES: *NCCC(=O)N1Cc2ccccc2-c2nnn(*)c2-c2ccccc21] |
| or | or |
| 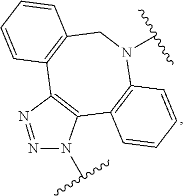 [SMILES: *N1Cc2ccccc2-c2nnn(*)c2-c2ccccc21] | 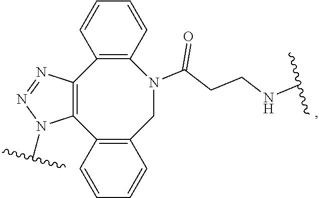 [SMILES: *NCCC(=O)N1Cc2ccccc2-c2c(nnn2*)-c2ccccc21] |
| 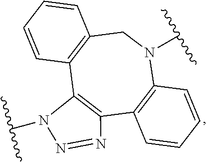 [SMILES: *N1Cc2ccccc2-c2c(nnn2*)-c2ccccc21] | 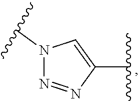 [SMILES: *c1cn(*)nn1] |
| or | or |
| 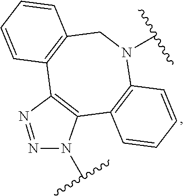 [SMILES: *N1Cc2ccccc2-c2nnn(*)c2-c2ccccc21] | 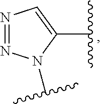 [SMILES: *c1cnnn1*] |
| 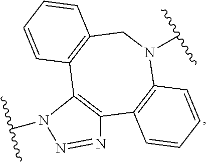 [SMILES: *N1Cc2ccccc2-c2c(nnn2*)-c2ccccc21] | 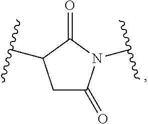 [SMILES: *C1CC(=O)N(*)C1=O] |
| or | |
| 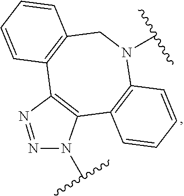 [SMILES: *N1Cc2ccccc2-c2nnn(*)c2-c2ccccc21] | |
| 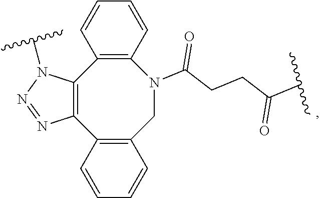 [SMILES: *C(=O)CCC(=O)N1Cc2ccccc2-c2nnn(*)c2-c2ccccc21] | 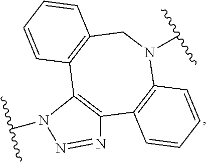 [SMILES: *N1Cc2ccccc2-c2c(nnn2*)-c2ccccc21] |
| or | or |
| 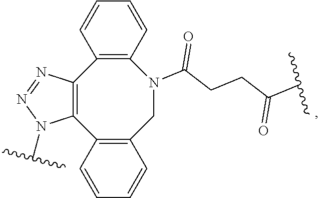 [SMILES: *C(=O)CCC(=O)N1Cc2ccccc2-c2c(nnn2*)-c2ccccc21] | 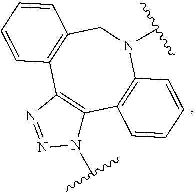 [SMILES: *N1Cc2ccccc2-c2nnn(*)c2-c2ccccc21] |
| 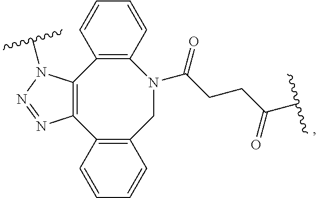 [SMILES: *C(=O)CCC(=O)N1Cc2ccccc2-c2nnn(*)c2-c2ccccc21] | 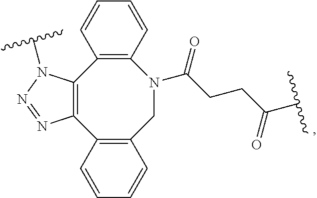 [SMILES: *C(=O)CCC(=O)N1Cc2ccccc2-c2nnn(*)c2-c2ccccc21] |
| or | or |
| 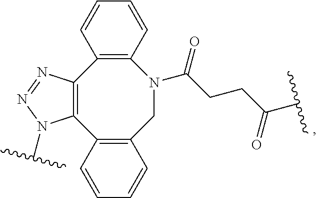 [SMILES: *C(=O)CCC(=O)N1Cc2ccccc2-c2c(nnn2*)-c2ccccc21] | 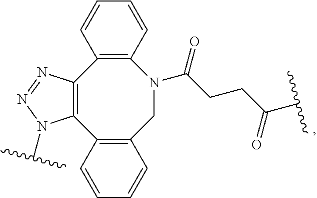 [SMILES: *C(=O)CCC(=O)N1Cc2ccccc2-c2c(nnn2*)-c2ccccc21] |
| 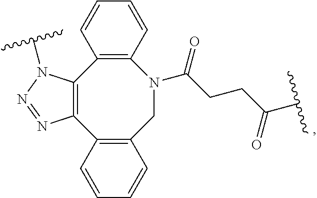 [SMILES: *C(=O)CCC(=O)N1Cc2ccccc2-c2nnn(*)c2-c2ccccc21] | 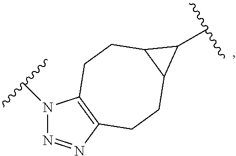 [SMILES: *C1C2CCc3nnn(*)c3CCC12] |
| or | |
| 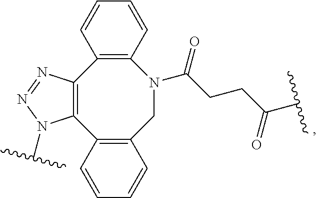 [SMILES: *C(=O)CCC(=O)N1Cc2ccccc2-c2c(nnn2*)-c2ccccc21] | 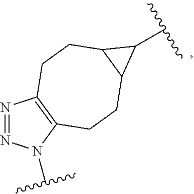 [SMILES: *C1C2CCc3nnn(*)c3CCC12] |
| 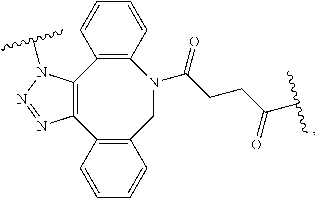 [SMILES: *C(=O)CCC(=O)N1Cc2ccccc2-c2nnn(*)c2-c2ccccc21] | 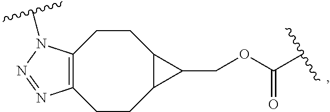 [SMILES: *C(=O)OCC1C2CCc3nnn(*)c3CCC21] |
| or | |
| 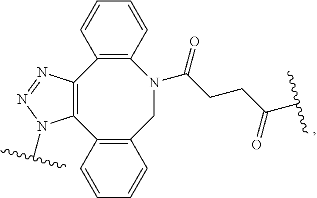 [SMILES: *C(=O)CCC(=O)N1Cc2ccccc2-c2c(nnn2*)-c2ccccc21] | 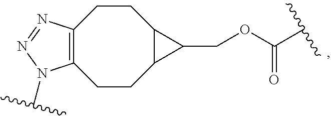 [SMILES: *C(=O)OCC1C2CCc3nnn(*)c3CCC21] |
| | 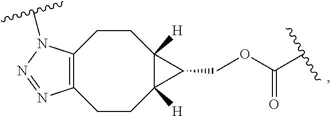 [SMILES: *C(=O)OC[C@@H]1[C@@H]2CCc3nnn(*)c3CC[C@@H]21] |
| | or |
| | 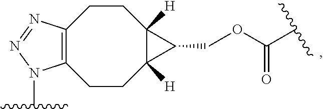 [SMILES: *C(=O)OC[C@@H]1[C@@H]2CCc3c(nnn3*)CC[C@@H]21] |
| 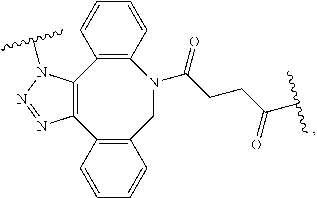 [SMILES: *C(=O)CCC(=O)N1Cc2ccccc2-c2nnn(*)c2-c2ccccc21] | 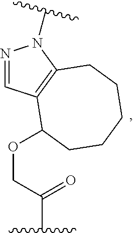 [SMILES: *C(=O)COC1CCCCCc2c1cnn2*] |
| or | or |
| 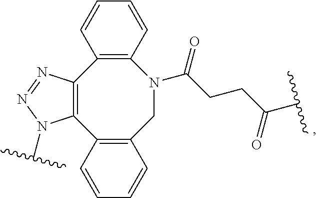 [SMILES: *C(=O)CCC(=O)N1Cc2ccccc2-c2c(nnn2*)-c2ccccc21] | 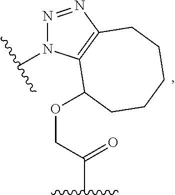 [SMILES: *C(=O)COC1CCCCCc2nnn(*)c21] |
| 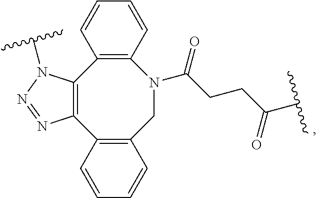 [SMILES: *C(=O)CCC(=O)N1Cc2ccccc2-c2nnn(*)c2-c2ccccc21] | 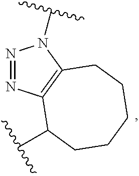 [SMILES: *C1CCCCCc2c1nnn2*] |
| or | or |
| 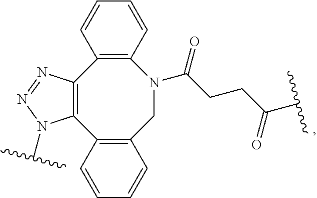 [SMILES: *C(=O)CCC(=O)N1Cc2ccccc2-c2c(nnn2*)-c2ccccc21] | 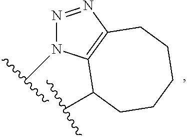 [SMILES: *C1CCCCCc2nnn(*)c21] |
| 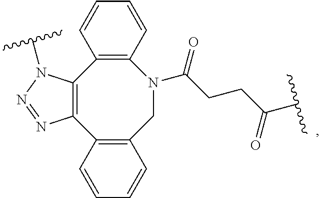 [SMILES: *C(=O)CCC(=O)N1Cc2ccccc2-c2nnn(*)c2-c2ccccc21] | 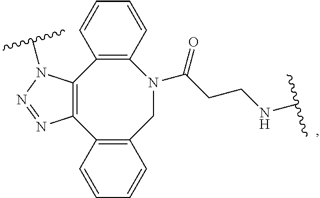 [SMILES: *NCCC(=O)N1Cc2ccccc2-c2nnn(*)c2-c2ccccc21] |
| or | or |
| 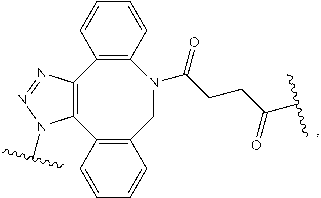 [SMILES: *C(=O)CCC(=O)N1Cc2ccccc2-c2c(nnn2*)-c2ccccc21] | 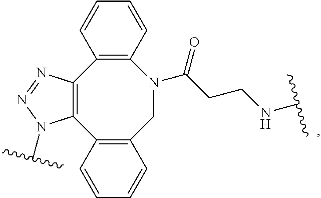 [SMILES: *NCCC(=O)N1Cc2ccccc2-c2c(nnn2*)-c2ccccc21] |
| 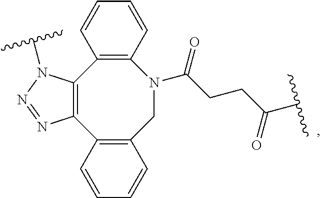 [SMILES: *C(=O)CCC(=O)N1Cc2ccccc2-c2nnn(*)c2-c2ccccc21] | 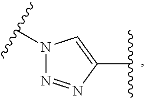 [SMILES: *c1cn(*)nn1] |
| or | or |
| 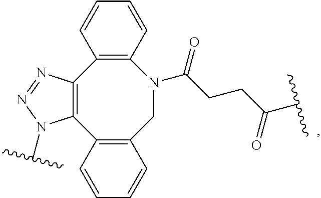 [SMILES: *C(=O)CCC(=O)N1Cc2ccccc2-c2c(nnn2*)-c2ccccc21] | 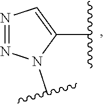 [SMILES: *c1cnnn1*] |
| 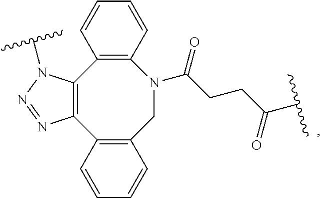 [SMILES: *C(=O)CCC(=O)N1Cc2ccccc2-c2nnn(*)c2-c2ccccc21] | 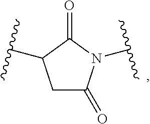 [SMILES: *C1CC(=O)N(*)C1=O] |
| or | |
| 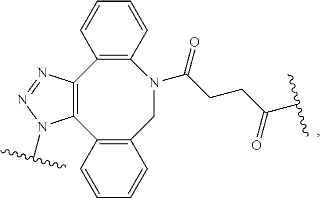 [SMILES: *C(=O)CCC(=O)N1Cc2ccccc2-c2c(nnn2*)-c2ccccc21] | |
| 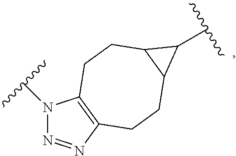 [SMILES: *C1C2CCc3nnn(*)c3CCC12] | 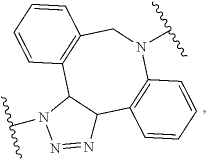 [SMILES: *N1Cc2ccccc2C2C(N=NN2*)c2ccccc21] |
| | or |
| 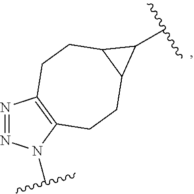 [SMILES: *C1C2CCc3nnn(*)c3CCC12] | 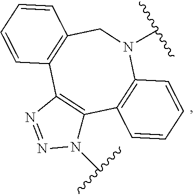 [SMILES: *N1Cc2ccccc2-c2nnn(*)c2-c2ccccc21] |
| 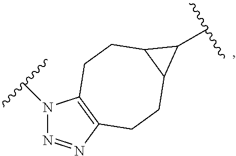 [SMILES: *C1C2CCc3nnn(*)c3CCC12] | 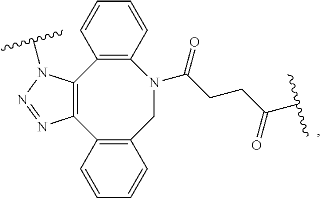 [SMILES: *C(=O)CCC(=O)N1Cc2ccccc2-c2nnn(*)c2-c2ccccc21] |
| | or |
| 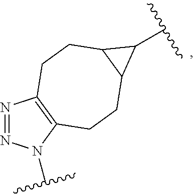 [SMILES: *C1C2CCc3nnn(*)c3CCC12] | 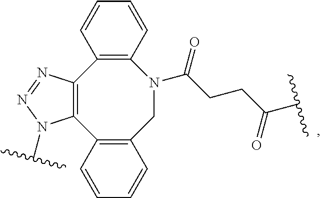 [SMILES: *C(=O)CCC(=O)N1Cc2ccccc2-c2c(nnn2*)-c2ccccc21] |
| 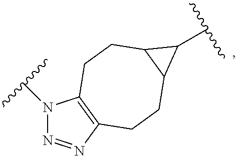 [SMILES: *C1C2CCc3nnn(*)c3CCC12] | 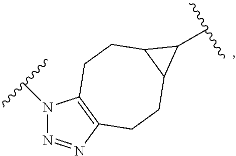 [SMILES: *C1C2CCc3nnn(*)c3CCC12] |
| 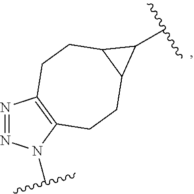 [SMILES: *C1C2CCc3nnn(*)c3CCC12] | 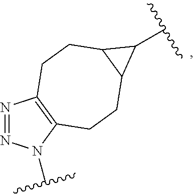 [SMILES: *C1C2CCc3nnn(*)c3CCC12] |
| 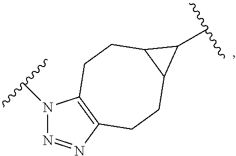 [SMILES: *C1C2CCc3nnn(*)c3CCC12] | 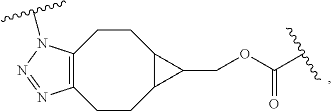 [SMILES: *C(=O)OCC1C2CCc3nnn(*)c3CCC21] |
| 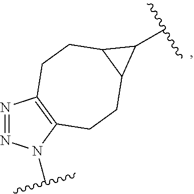 [SMILES: *C1C2CCc3nnn(*)c3CCC12] | 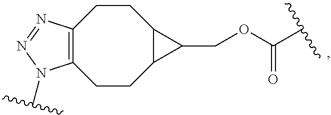 [SMILES: *C(=O)OCC1C2CCc3nnn(*)c3CCC21] |
| | 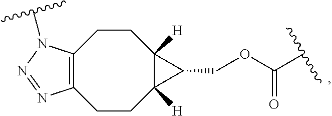 [SMILES: *C(=O)OC[C@@H]1[C@@H]2CCc3nnn(*)c3CC[C@@H]21] |
| | or |
| | 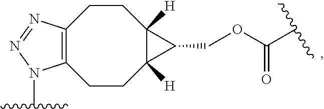 [SMILES: *C(=O)OC[C@@H]1[C@@H]2CCc3c(nnn3*)CC[C@@H]21] |
| 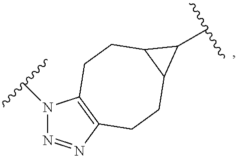 [SMILES: *C1C2CCc3nnn(*)c3CCC12] | 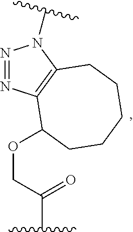 [SMILES: *C(=O)COC1CCCCCc2c1nnn2*] |
| | or |
| 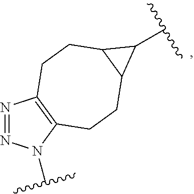 [SMILES: *C1C2CCc3nnn(*)c3CCC12] | 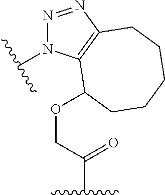 [SMILES: *C(=O)COC1CCCCCc2nnn(*)c21] |
| 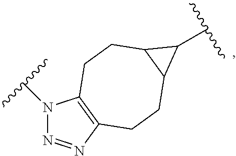 [SMILES: *C1C2CCc3nnn(*)c3CCC12] | 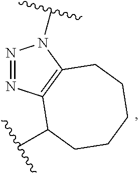 [SMILES: *C1CCCCCc2c1nnn2*] |
| | or |
| 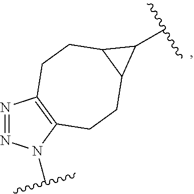 [SMILES: *C1C2CCc3nnn(*)c3CCC12] | 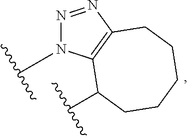 [SMILES: *C1CCCCCc2nnn(*)c21] |
| 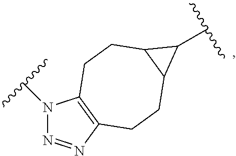 [SMILES: *C1C2CCc3nnn(*)c3CCC12] | 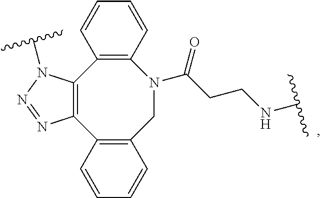 [SMILES: *NCCC(=O)N1Cc2ccccc2-c2nnn(*)c2-c2ccccc21] |
| | or |
| 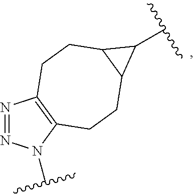 [SMILES: *C1C2CCc3nnn(*)c3CCC12] | 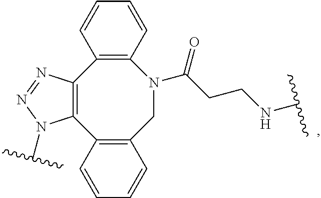 [SMILES: *NCCC(=O)N1Cc2ccccc2-c2c(nnn2*)-c2ccccc21] |
| 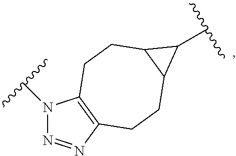 [SMILES: *C1C2CCc3nnn(*)c3CCC12] | 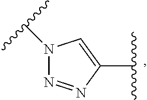 [SMILES: *c1cn(*)nn1] |
| | or |
| 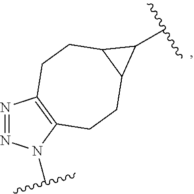 [SMILES: *C1C2CCc3nnn(*)c3CCC12] | 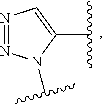 [SMILES: *c1cnnn1*] |
| 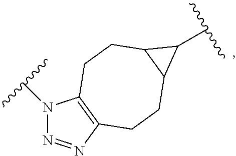 [SMILES: *C1C2CCc3nnn(*)c3CCC12] | 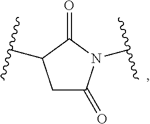 [SMILES: *C1CC(=O)N(*)C1=O] |
| 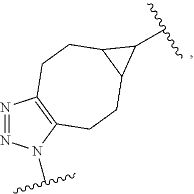 [SMILES: *C1C2CCc3nnn(*)c3CCC12] | |
| 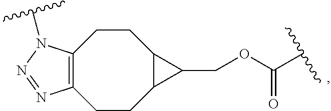 [SMILES: *C(=O)OCC1C2CCc3nnn(*)c3CCC21] | 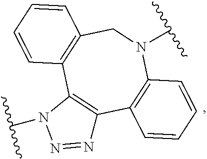 [SMILES: *N1Cc2ccccc2-c2c(nnn2*)-c2ccccc21] |
| | or |
| 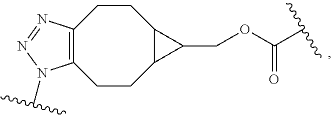 [SMILES: *C(=O)OCC1C2CCc3nnn(*)c3CCC21] | 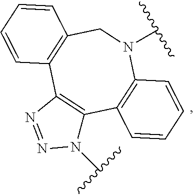 [SMILES: *N1Cc2ccccc2-c2nnn(*)c2-c2ccccc21] |
| 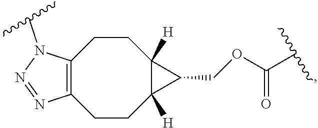 [SMILES: *C(=O)OC[C@@H]1[C@@H]2CCc3nnn(*)c3CC[C@@H]21] | |
| or | |
| 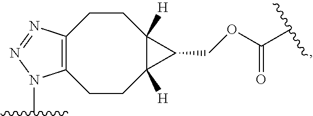 [SMILES: *C(=O)OC[C@@H]1[C@@H]2CCc3c(nnn3*)CC[C@@H]21] | |
| 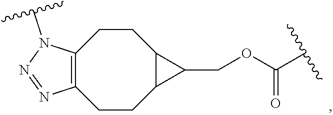 [SMILES: *C(=O)OCC1C2CCc3nnn(*)c3CCC21] | 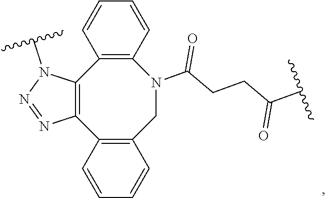 [SMILES: *C(=O)CCC(=O)N1Cc2ccccc2-c2nnn(*)c2-c2ccccc21] |
| | or |
| 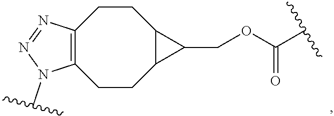 [SMILES: *C(=O)OCC1C2CCc3nnn(*)c3CCC21] | 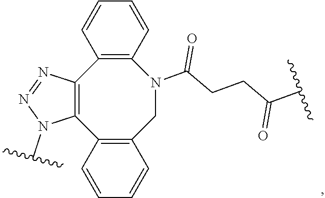 [SMILES: *C(=O)CCC(=O)N1Cc2ccccc2-c2c(nnn2*)-c2ccccc21] |
| 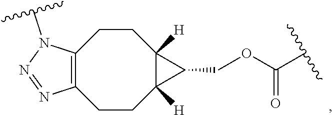 [SMILES: *C(=O)OC[C@@H]1[C@@H]2CCc3nnn(*)c3CC[C@@H]21] | |
| or | |
| 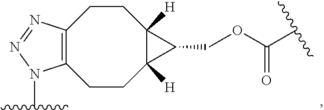 [SMILES: *C(=O)OC[C@@H]1[C@@H]2CCc3c(nnn3*)CC[C@@H]21] | |
| 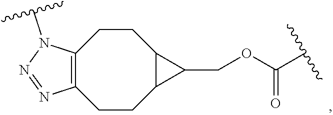 [SMILES: *C(=O)OCC1C2CCc3nnn(*)c3CCC21] | 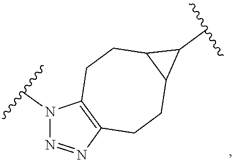 [SMILES: *C1C2CCc3nnn(*)c3CCC12] |
| 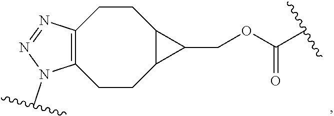 [SMILES: *C(=O)OCC1C2CCc3nnn(*)c3CCC21] | 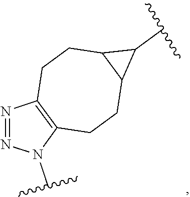 [SMILES: *C1C2CCc3nnn(*)c3CCC12] |
| 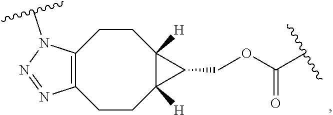 [SMILES: *C(=O)OC[C@@H]1[C@@H]2CCc3nnn(*)c3CC[C@@H]21] | |
| or | |
| 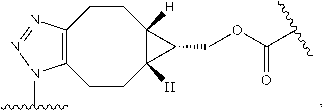 [SMILES: *C(=O)OC[C@@H]1[C@@H]2CCc3c(nnn3*)CC[C@@H]21] | |
| 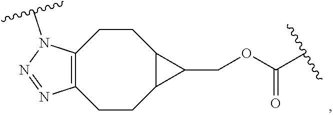 [SMILES: *C(=O)OCC1C2CCc3nnn(*)c3CCC21] | 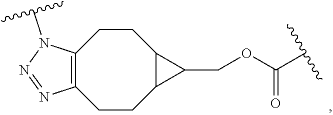 [SMILES: *C(=O)OCC1C2CCc3nnn(*)c3CCC21] |
| 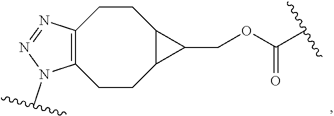 [SMILES: *C(=O)OCC1C2CCc3nnn(*)c3CCC21] | 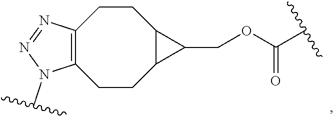 [SMILES: *C(=O)OCC1C2CCc3nnn(*)c3CCC21] |
| 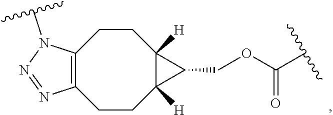 [SMILES: *C(=O)OC[C@@H]1[C@@H]2CCc3nnn(*)c3CC[C@@H]21] | 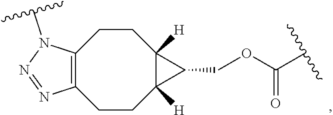 [SMILES: *C(=O)OC[C@@H]1[C@@H]2CCc3nnn(*)c3CC[C@@H]21] |
| or | or |
| 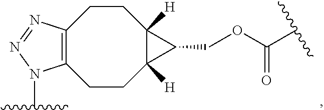 [SMILES: *C(=O)OC[C@@H]1[C@@H]2CCc3c(nnn3*)CC[C@@H]21] | 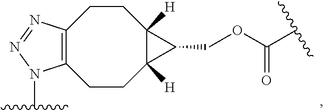 [SMILES: *C(=O)OC[C@@H]1[C@@H]2CCc3c(nnn3*)CC[C@@H]21] |
| 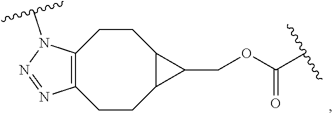 [SMILES: *C(=O)OCC1C2CCc3nnn(*)c3CCC21] | 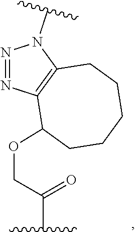 [SMILES: *C(=O)COC1CCCCCc2c1nnn2*] |
| | or |
| 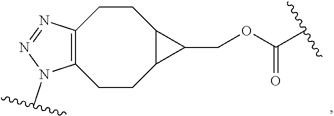 [SMILES: *C(=O)OCC1C2CCc3nnn(*)c3CCC21] | 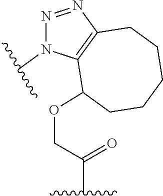 [SMILES: *C(=O)COC1CCCCCc2nnn(*)c21] |
| 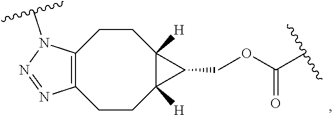 [SMILES: *C(=O)OC[C@@H]1[C@@H]2CCc3nnn(*)c3CC[C@@H]21] | |
| or | |
| 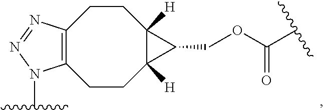 [SMILES: *C(=O)OC[C@@H]1[C@@H]2CCc3c(nnn3*)CC[C@@H]21] | |
| 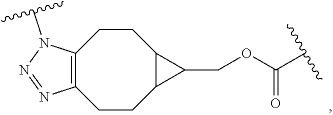 [SMILES: *C(=O)OCC1C2CCc3nnn(*)c3CCC21] | 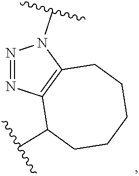 [SMILES: *C1CCCCCc2c1nnn2*] |
| | or |
| 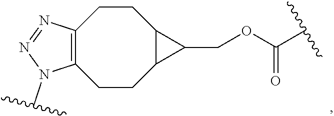 [SMILES: *C(=O)OCC1C2CCc3nnn(*)c3CCC21] | 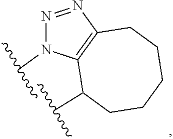 [SMILES: *C1CCCCCc2nnn(*)c21] |
| 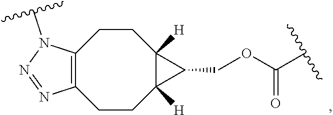 [SMILES: *C(=O)OC[C@@H]1[C@@H]2CCc3nnn(*)c3CC[C@@H]21] | |
| or | |
| 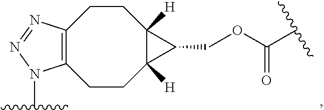 [SMILES: *C(=O)OC[C@@H]1[C@@H]2CCc3c(nnn3*)CC[C@@H]21] | |
| 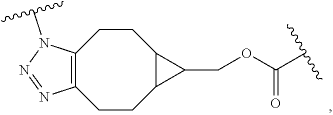 [SMILES: *C(=O)OCC1C2CCc3nnn(*)c3CCC21] | 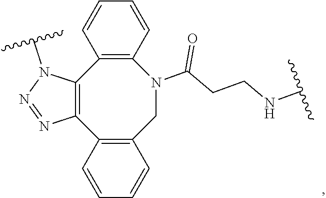 [SMILES: *NCCC(=O)N1Cc2ccccc2-c2nnn(*)c2-c2ccccc21] |
| | or |
| 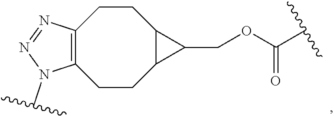 [SMILES: *C(=O)OCC1C2CCc3nnn(*)c3CCC21] | 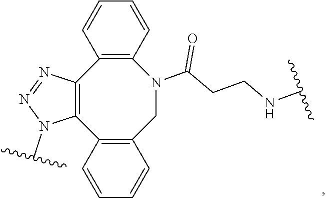 [SMILES: *NCCC(=O)N1Cc2ccccc2-c2c(nnn2*)-c2ccccc21] |
| 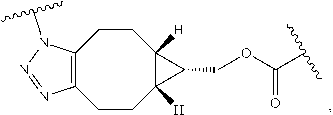 [SMILES: *C(=O)OC[C@@H]1[C@@H]2CCc3nnn(*)c3CC[C@@H]21] | |
| or | |
| 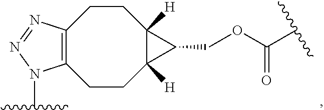 [SMILES: *C(=O)OC[C@@H]1[C@@H]2CCc3c(nnn3*)CC[C@@H]21] | |
| 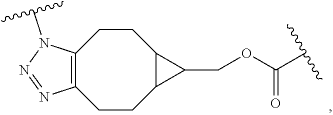 [SMILES: *C(=O)OCC1C2CCc3nnn(*)c3CCC21] | 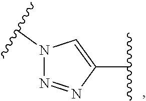 [SMILES: *c1cn(*)nn1] |
| | or |
| 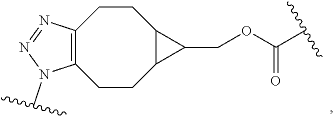 [SMILES: *C(=O)OCC1C2CCc3nnn(*)c3CCC21] | 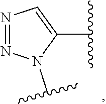 [SMILES: *c1cnnn1*] |
| 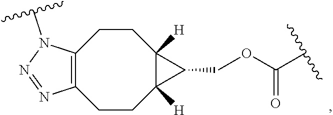 [SMILES: *C(=O)OC[C@@H]1[C@@H]2CCc3nnn(*)c3CC[C@@H]21] | |
| or | |
| 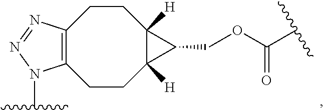 [SMILES: *C(=O)OC[C@@H]1[C@@H]2CCc3c(nnn3*)CC[C@@H]21] | |
| 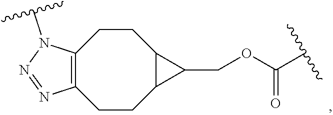 [SMILES: *C(=O)OCC1C2CCc3nnn(*)c3CCC21] | 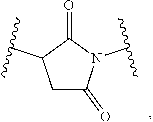 [SMILES: *C1CC(=O)N(*)C1=O] |
| 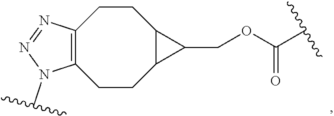 [SMILES: *C(=O)OCC1C2CCc3nnn(*)c3CCC21] | |
| 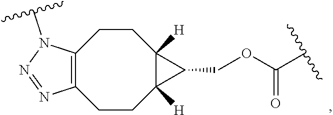 [SMILES: *C(=O)OC[C@@H]1[C@@H]2CCc3nnn(*)c3CC[C@@H]21] | |
| or | |
| 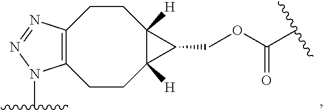 [SMILES: *C(=O)OC[C@@H]1[C@@H]2CCc3c(nnn3*)CC[C@@H]21] | |
| 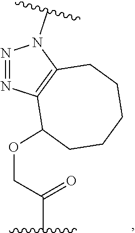 [SMILES: *C(=O)COC1CCCCCc2c1nnn2*] | 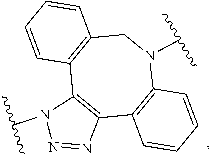 [SMILES: *N1Cc2ccccc2-c2c(nnn2*)-c2ccccc21] |
| or | or |
| 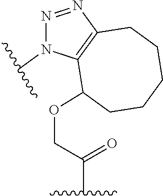 [SMILES: *C(=O)COC1CCCCCc2nnn(*)c21] | 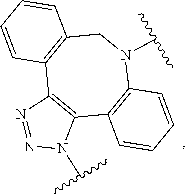 [SMILES: *N1Cc2ccccc2-c2nnn(*)c2-c2ccccc21] |
| 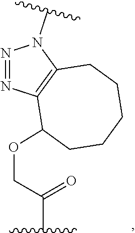 [SMILES: *C(=O)COC1CCCCCc2c1nnn2*] | 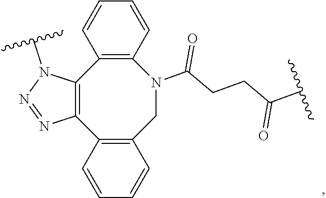 [SMILES: *C(=O)CCC(=O)N1Cc2ccccc2-c2nnn(*)c2-c2ccccc21] |
| or | or |
| 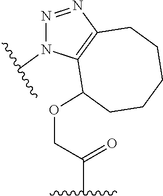 [SMILES: *C(=O)COC1CCCCCc2nnn(*)c21] | 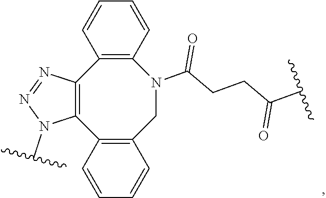 [SMILES: *C(=O)CCC(=O)N1Cc2ccccc2-c2c(nnn2*)-c2ccccc21] |
| 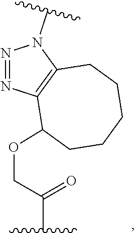 [SMILES: *C(=O)COC1CCCCCc2c1nnn2*] | 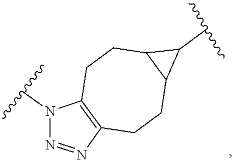 [SMILES: *C1C2CCc3nnn(*)c3CCC12] |
| 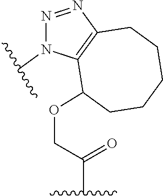 [SMILES: *C(=O)COC1CCCCCc2nnn(*)c21] | 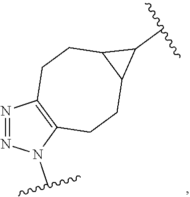 [SMILES: *C1C2CCc3nnn(*)c3CCC12] |
| 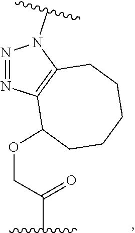 [SMILES: *C(=O)COC1CCCCCc2c1nnn2*] | 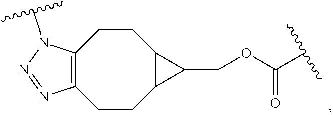 [SMILES: *C(=O)OCC1C2CCc3nnn(*)c3CCC21] |
| or | |
| 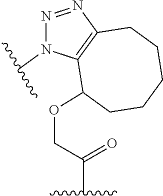 [SMILES: *C(=O)COC1CCCCCc2nnn(*)c21] | 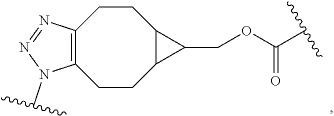 [SMILES: *C(=O)OCC1C2CCc3nnn(*)c3CCC21] |
| | 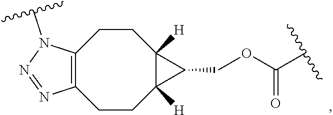 [SMILES: *C(=O)OC[C@@H]1[C@@H]2CCc3nnn(*)c3CC[C@@H]21] |
| | or |
| | 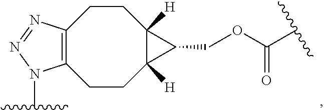 [SMILES: *C(=O)OC[C@@H]1[C@@H]2CCc3c(nnn3*)CC[C@@H]21] |
| 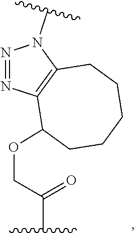 [SMILES: *C(=O)COC1CCCCCc2c1nnn2*] | 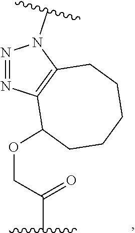 [SMILES: *C(=O)COC1CCCCCc2c1nnn2*] |
| or | or |
| 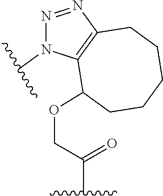 [SMILES: *C(=O)COC1CCCCCc2nnn(*)c21] | 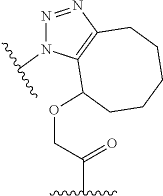 [SMILES: *C(=O)COC1CCCCCc2nnn(*)c21] |
| 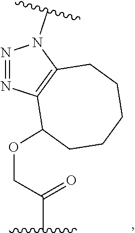 [SMILES: *C(=O)COC1CCCCCc2c1nnn2*] | 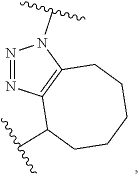 [SMILES: *C1CCCCCc2c1nnn2*] |
| or | or |
| 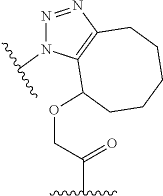 [SMILES: *C(=O)COC1CCCCCc2nnn(*)c21] | 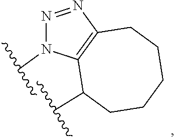 [SMILES: *C1CCCCCc2nnn(*)c21] |
| 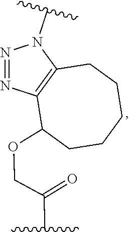 [SMILES: *C(=O)COC1CCCCCc2c1nnn2*] | 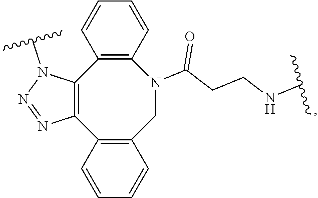 [SMILES: *NCCC(=O)N1Cc2ccccc2-c2nnn(*)c2-c2ccccc21] |
| or | or |
| 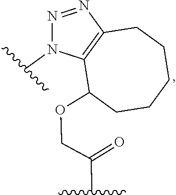 [SMILES: *C(=O)COC1CCCCCc2nnn(*)c21] | 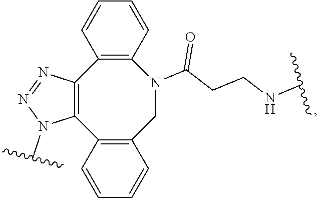 [SMILES: *NCCC(=O)N1Cc2ccccc2-c2c(nnn2*)-c2ccccc21] |
| 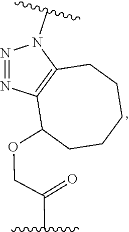 [SMILES: *C(=O)COC1CCCCCc2c1nnn2*] | 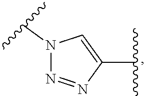 [SMILES: *c1cn(*)nn1] |
| or | or |
| 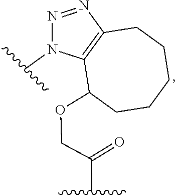 [SMILES: *C(=O)COC1CCCCCc2nnn(*)c21] | 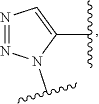 [SMILES: *c1cnnn1*] |
| 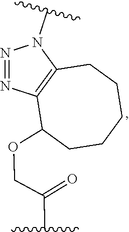 [SMILES: *C(=O)COC1CCCCCc2c1nnn2*] | 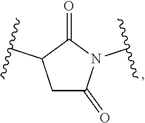 [SMILES: *C1CC(=O)N(*)C1=O] |
| or | |
| 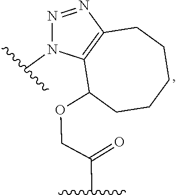 [SMILES: *C(=O)COC1CCCCCc2nnn(*)c21] | |
| 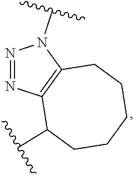 [SMILES: *C1CCCCCc2c1nnn2*] | 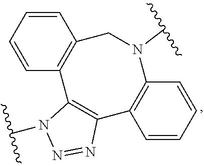 [SMILES: *N1Cc2ccccc2-c2c(nnn2*)-c2ccccc21] |
| or | or |
| 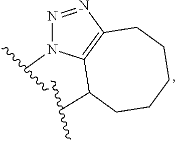 [SMILES: *C1CCCCCc2nnn(*)c21] | 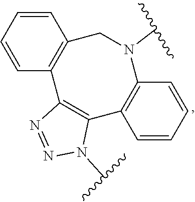 [SMILES: *N1Cc2ccccc2-c2nnn(*)c2-c2ccccc21] |
| 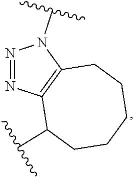 [SMILES: *C1CCCCCc2c1nnn2*] | 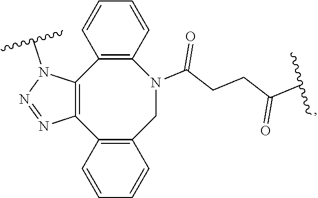 [SMILES: *C(=O)CCC(=O)N1Cc2ccccc2-c2nnn(*)c2-c2ccccc21] |
| or | or |
| 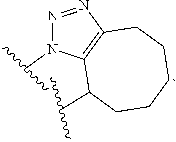 [SMILES: *C1CCCCCc2nnn(*)c21] | 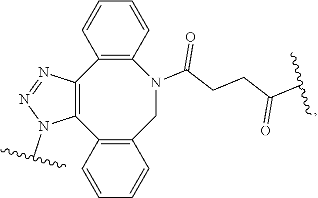 [SMILES: *C(=O)CCC(=O)N1Cc2ccccc2-c2c(nnn2*)-c2ccccc21] |
| 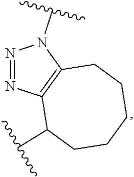 [SMILES: *C1CCCCCc2c1nnn2*] | 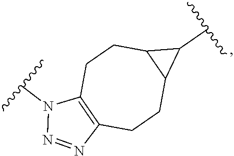 [SMILES: *C1C2CCc3nnn(*)c3CCC12] |
| or | |
| 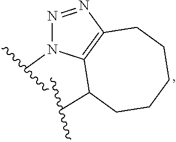 [SMILES: *C1CCCCCc2nnn(*)c21] | 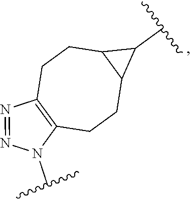 [SMILES: *C1C2CCc3nnn(*)c3CCC12] |
| 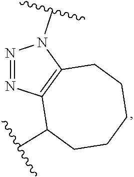 [SMILES: *C1CCCCCc2c1nnn2*] | 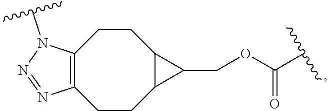 [SMILES: *C(=O)OCC1C2CCc3nnn(*)c3CCC21] |
| or | |
| 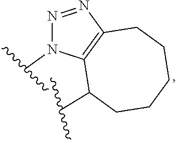 [SMILES: *C1CCCCCc2nnn(*)c21] | 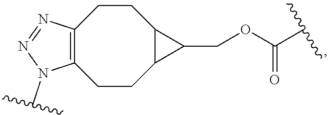 [SMILES: *C(=O)OCC1C2CCc3nnn(*)c3CCC21] |
| | 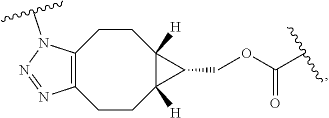 [SMILES: *C(=O)OC[C@@H]1[C@@H]2CCc3nnn(*)c3CC[C@@H]21] |
| | or |
| | 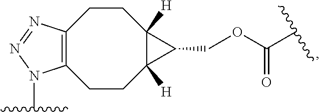 [SMILES: *C(=O)OC[C@@H]1[C@@H]2CCc3c(nnn3*)CC[C@@H]21] |
| 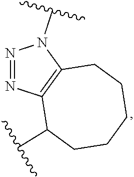 [SMILES: *C1CCCCCc2c1nnn2*] | 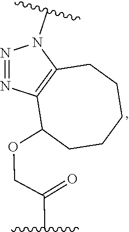 [SMILES: *C(=O)COC1CCCCCc2c1nnn2*] |
| or | or |
| 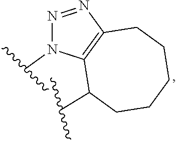 [SMILES: *C1CCCCCc2nnn(*)c21] | 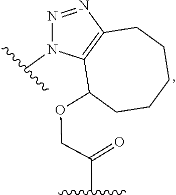 [SMILES: *C(=O)COC1CCCCCc2nnn(*)c21] |
| 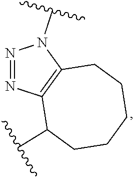 [SMILES: *C1CCCCCc2c1nnn2*] | 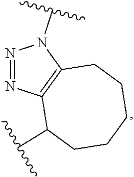 [SMILES: *C1CCCCCc2c1nnn2*] |
| or | or |
| 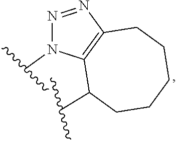 [SMILES: *C1CCCCCc2nnn(*)c21] | 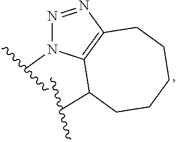 [SMILES: *C1CCCCCc2nnn(*)c21] |
| 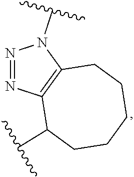 [SMILES: *C1CCCCCc2c1nnn2*] | 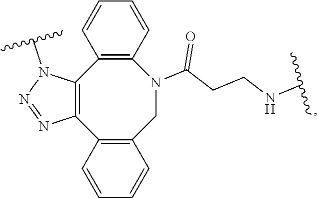 [SMILES: *NCCC(=O)N1Cc2ccccc2-c2nnn(*)c2-c2ccccc21] |
| or | or |
| 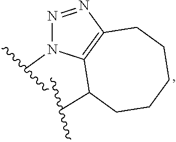 [SMILES: *C1CCCCCc2nnn(*)c21] | 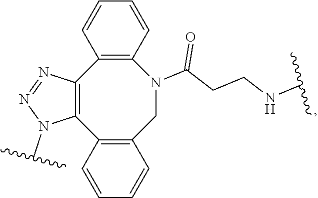 [SMILES: *NCCC(=O)N1Cc2ccccc2-c2c(nnn2*)-c2ccccc21] |
| 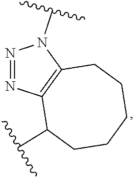 [SMILES: *C1CCCCCc2c1nnn2*] | 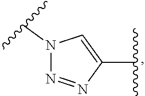 [SMILES: *c1cn(*)nn1] |
| or | or |
| 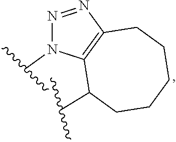 [SMILES: *C1CCCCCc2nnn(*)c21] | 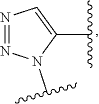 [SMILES: *c1cnnn1*] |
| 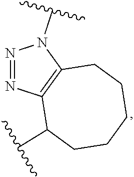 [SMILES: *C1CCCCCc2c1nnn2*] | 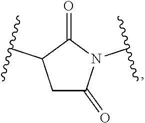 [SMILES: *C1CC(=O)N(*)C1=O] |
| or | |
| 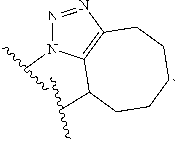 [SMILES: *C1CCCCCc2nnn(*)c21] | |
| 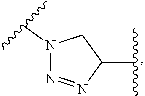 [SMILES: *C1CN(*)N=N1] | 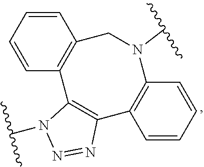 [SMILES: *N1Cc2ccccc2-c2c(nnn2*)-c2ccccc21] |
| or | or |
| 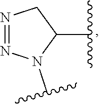 [SMILES: *C1CN=NN1*] | 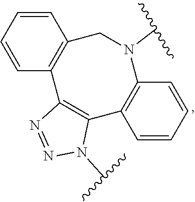 [SMILES: *N1Cc2ccccc2-c2nnn(*)c2-c2ccccc21] |
| 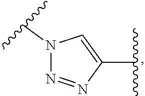 [SMILES: *c1cn(*)nn1] | 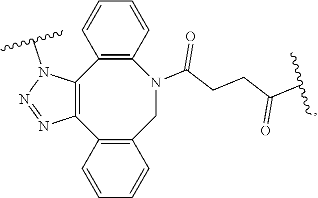 [SMILES: *C(=O)CCC(=O)N1Cc2ccccc2-c2nnn(*)c2-c2ccccc21] |
| or | or |
| 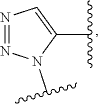 [SMILES: *c1cnnn1*] | 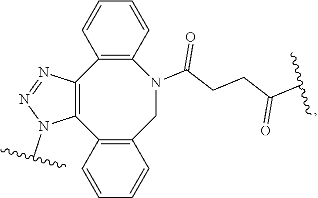 [SMILES: *C(=O)CCC(=O)N1Cc2ccccc2-c2c(nnn2*)-c2ccccc21] |
| 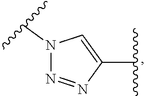 [SMILES: *c1cn(*)nn1] | 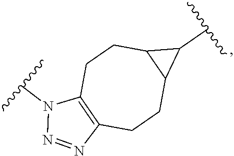 [SMILES: *C1C2CCc3nnn(*)c3CCC12] |
| or | |
| 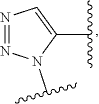 [SMILES: *c1cnnn1*] | 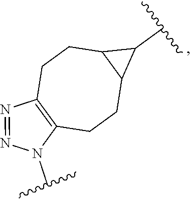 [SMILES: *C1C2CCc3nnn(*)c3CCC12] |
| 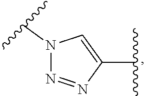 [SMILES: *c1cn(*)nn1] | 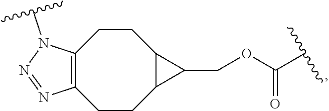 [SMILES: *C(=O)OCC1C2CCc3nnn(*)c3CCC21] |
| or | |
| 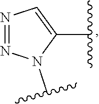 [SMILES: *c1cnnn1*] | 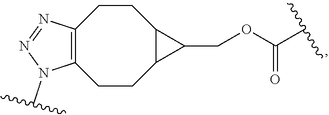 [SMILES: *C(=O)OCC1C2CCc3nnn(*)c3CCC21] |
| | 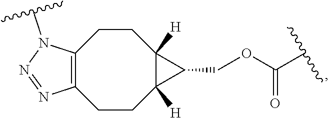 [SMILES: *C(=O)OC[C@@H]1[C@@H]2CCc3nnn(*)c3CC[C@@H]21] |
| | or |
| | 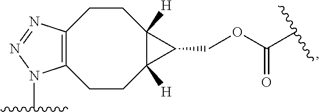 [SMILES: *C(=O)OC[C@@H]1[C@@H]2CCc3c(nnn3*)CC[C@@H]21] |
| 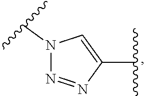 [SMILES: *c1cn(*)nn1] | 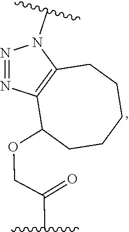 [SMILES: *C(=O)COC1CCCCCc2c1nnn2*] |
| or | or |
| 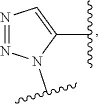 [SMILES: *c1cnnn1*] | 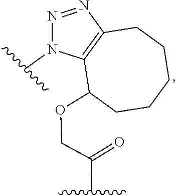 [SMILES: *C(=O)COC1CCCCCc2nnn(*)c21] |
| 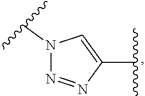 [SMILES: *c1cn(*)nn1] | 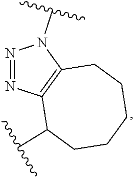 [SMILES: *C1CCCCCc2c1nnn2*] |
| or | or |
| 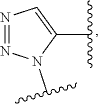 [SMILES: *c1cnnn1*] | 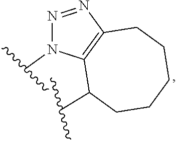 [SMILES: *C1CCCCCc2nnn(*)c21] |
| 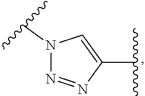 [SMILES: *c1cn(*)nn1] | 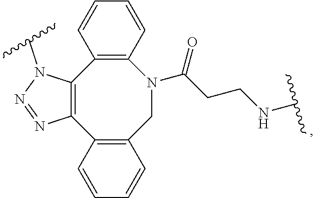 [SMILES: *NCCC(=O)N1Cc2ccccc2-c2nnn(*)c2-c2ccccc21] |
| or | or |
| 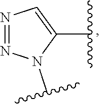 [SMILES: *c1cnnn1*] | 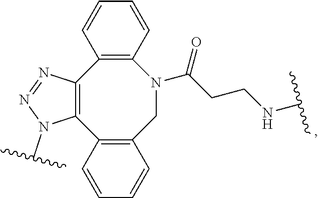 [SMILES: *NCCC(=O)N1Cc2ccccc2-c2c(nnn2*)-c2ccccc21] |
| 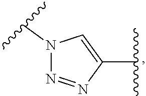 [SMILES: *c1cn(*)nn1] | 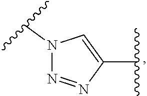 [SMILES: *c1cn(*)nn1] |
| or | or |
| 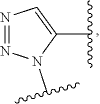 [SMILES: *c1cnnn1*] | 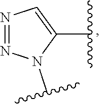 [SMILES: *c1cnnn1*] |
| 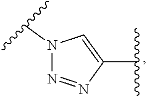 [SMILES: *c1cn(*)nn1] | 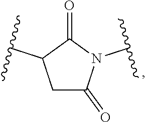 [SMILES: *C1CC(=O)N(*)C1=O] |
| or | |
| 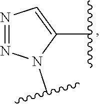 [SMILES: *c1cnnn1*] | |
| 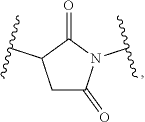 [SMILES: *C1CC(=O)N(*)C1=O] | 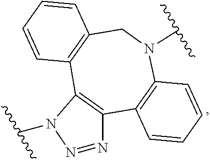 [SMILES: *N1Cc2ccccc2-c2c(nnn2*)-c2ccccc21] |
| | or |
| | 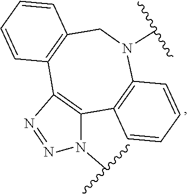 [SMILES: *N1Cc2ccccc2-c2nnn(*)c2-c2ccccc21] |
| 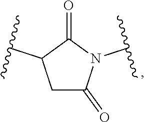 [SMILES: *C1CC(=O)N(*)C1=O] | 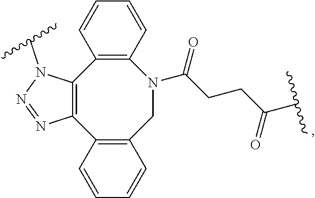 [SMILES: *C(=O)CCC(=O)N1Cc2ccccc2-c2nnn(*)c2-c2ccccc21] |
| | or |
| | 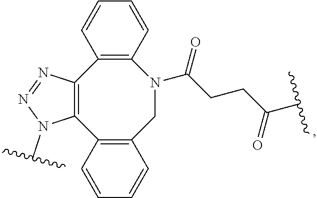 [SMILES: *C(=O)CCC(=O)N1Cc2ccccc2-c2c(nnn2*)-c2ccccc21] |
| 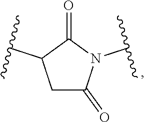 [SMILES: *C1CC(=O)N(*)C1=O] | 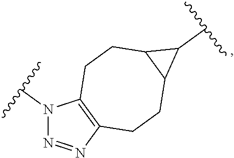 [SMILES: *C1C2CCc3nnn(*)c3CCC12] |
| | 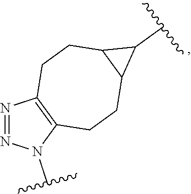 [SMILES: *C1C2CCc3nnn(*)c3CCC12] |
| 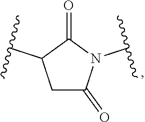 [SMILES: *C1CC(=O)N(*)C1=O] | 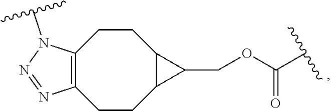 [SMILES: *C(=O)OCC1C2CCc3nnn(*)c3CCC21] |
| | 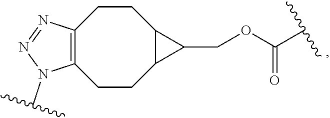 [SMILES: *C(=O)OCC1C2CCc3nnn(*)c3CCC21] |
| | 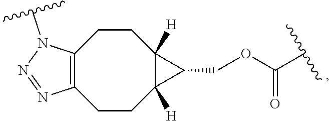 [SMILES: *C(=O)OC[C@@H]1[C@@H]2CCc3nnn(*)c3CC[C@@H]21] |
| | or |
| | 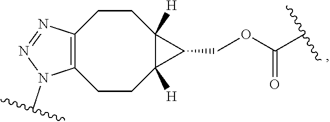 [SMILES: *C(=O)OC[C@@H]1[C@@H]2CCc3c(nnn3*)CC[C@@H]21] |
| 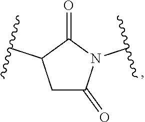 [SMILES: *C1CC(=O)N(*)C1=O] | 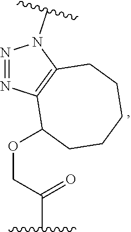 [SMILES: *C(=O)COC1CCCCCc2c1nnn2*] |
| | or |
| | 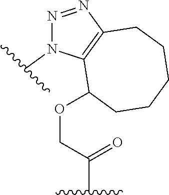 [SMILES: *C(=O)COC1CCCCCc2nnn(*)c21] |
| 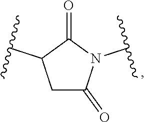 [SMILES: *C1CC(=O)N(*)C1=O] | 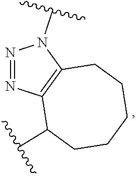 [SMILES: *C1CCCCCc2c1nnn2*] |
| | or |
| | 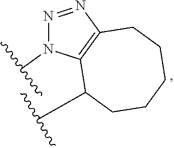 [SMILES: *C1CCCCCc2nnn(*)c21] |
| 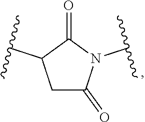 [SMILES: *C1CC(=O)N(*)C1=O] | 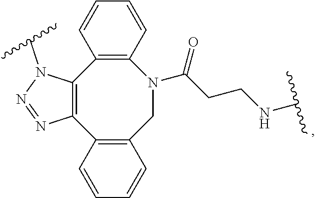 [SMILES: *NCCC(=O)N1Cc2ccccc2-c2nnn(*)c2-c2ccccc21] |
| | or |
| | 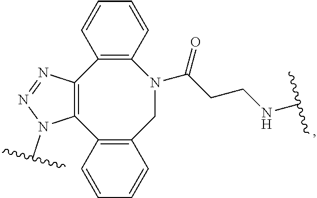 [SMILES: *NCCC(=O)N1Cc2ccccc2-c2c(nnn2*)-c2ccccc21] |
| 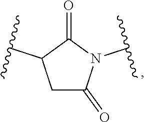 [SMILES: *C1CC(=O)N(*)C1=O] | 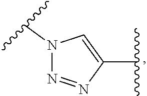 [SMILES: *c1cn(*)nn1] |
| | or |
| | 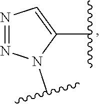 [SMILES: *c1cnnn1*] |
| 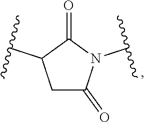 [SMILES: *C1CC(=O)N(*)C1=O] | 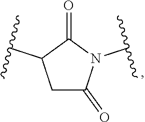 [SMILES: *C1CC(=O)N(*)C1=O] |
| 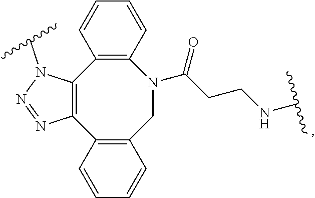 [SMILES: *NCCC(=O)N1Cc2ccccc2-c2nnn(*)c2-c2ccccc21] | 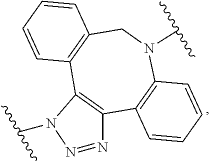 [SMILES: *N1Cc2ccccc2-c2c(nnn2*)-c2ccccc21] |
| or | or |
| 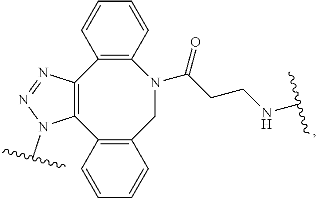 [SMILES: *NCCC(=O)N1Cc2ccccc2-c2c(nnn2*)-c2ccccc21] | 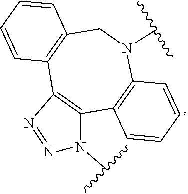 [SMILES: *N1Cc2ccccc2-c2nnn(*)c2-c2ccccc21] |
| 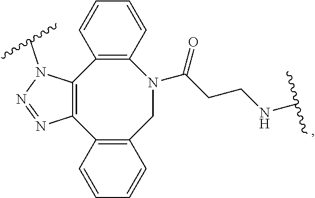 [SMILES: *NCCC(=O)N1Cc2ccccc2-c2nnn(*)c2-c2ccccc21] | 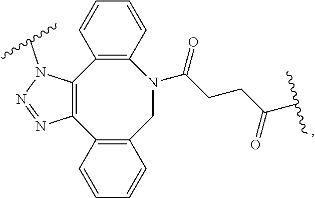 [SMILES: *C(=O)CCC(=O)N1Cc2ccccc2-c2nnn(*)c2-c2ccccc21] |
| or | or |
| 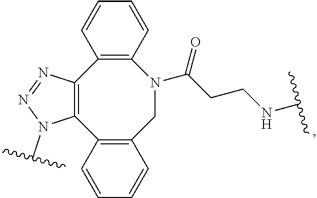 [SMILES: *NCCC(=O)N1Cc2ccccc2-c2c(nnn2*)-c2ccccc21] | 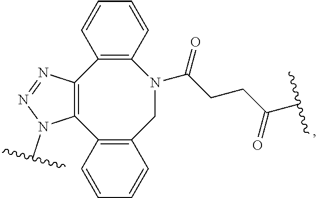 [SMILES: *C(=O)CCC(=O)N1Cc2ccccc2-c2c(nnn2*)-c2ccccc21] |
| 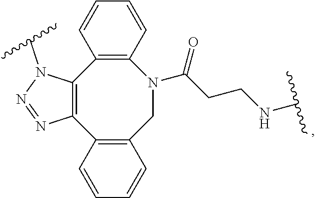 [SMILES: *NCCC(=O)N1Cc2ccccc2-c2nnn(*)c2-c2ccccc21] | 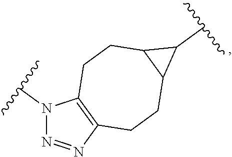 [SMILES: *C1C2CCc3nnn(*)c3CCC12] |
| or | |
| 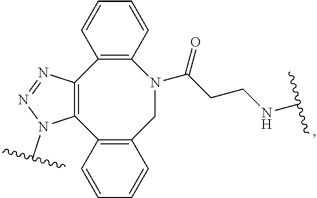 [SMILES: *NCCC(=O)N1Cc2ccccc2-c2c(nnn2*)-c2ccccc21] | 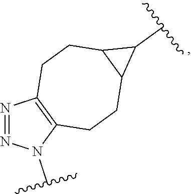 [SMILES: *C1C2CCc3nnn(*)c3CCC12] |
| 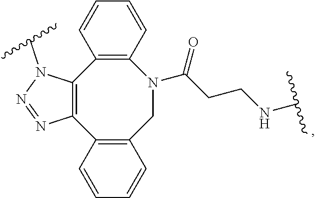 [SMILES: *NCCC(=O)N1Cc2ccccc2-c2nnn(*)c2-c2ccccc21] | 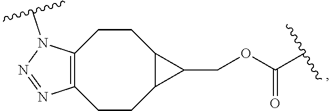 [SMILES: *C(=O)OCC1C2CCc3nnn(*)c3CCC21] |
| or | |
| 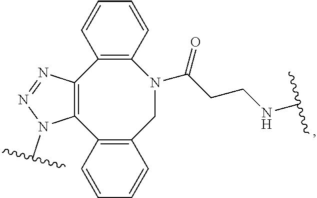 [SMILES: *NCCC(=O)N1Cc2ccccc2-c2c(nnn2*)-c2ccccc21] | 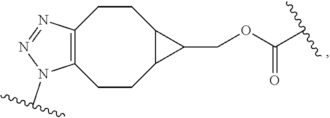 [SMILES: *C(=O)OCC1C2CCc3nnn(*)c3CCC21] |
| | 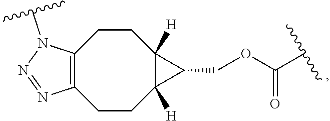 [SMILES: *C(=O)OC[C@@H]1[C@@H]2CCc3nnn(*)c3CC[C@@H]21] |
| | or |
| | 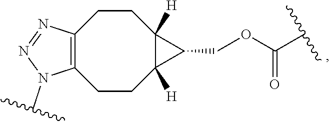 [SMILES: *C(=O)OC[C@@H]1[C@@H]2CCc3c(nnn3*)CC[C@@H]21] |
| 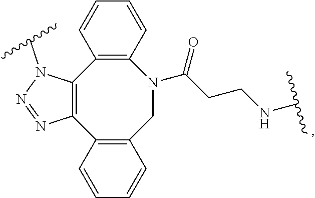 [SMILES: *NCCC(=O)N1Cc2ccccc2-c2nnn(*)c2-c2ccccc21] | 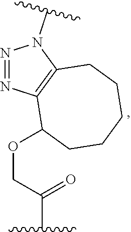 [SMILES: *C(=O)COC1CCCCCc2c1nnn2*] |
| or | or |
| 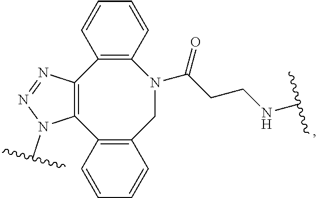 [SMILES: *NCCC(=O)N1Cc2ccccc2-c2c(nnn2*)-c2ccccc21] | 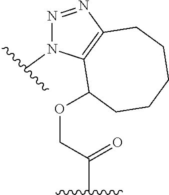 [SMILES: *C(=O)COC1CCCCCc2nnn(*)c21] |
| 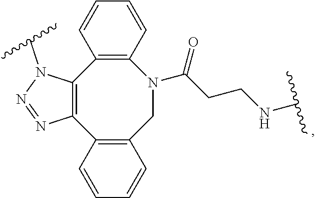 [SMILES: *NCCC(=O)N1Cc2ccccc2-c2nnn(*)c2-c2ccccc21] | 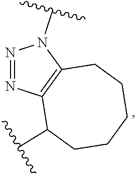 [SMILES: *C1CCCCCc2c1nnn2*] |
| or | or |
| 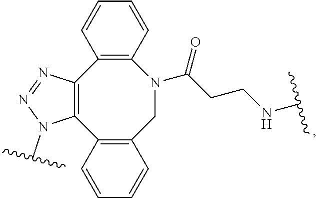 [SMILES: *NCCC(=O)N1Cc2ccccc2-c2c(nnn2*)-c2ccccc21] | 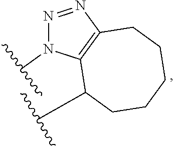 [SMILES: *C1CCCCCc2nnn(*)c21] |
| 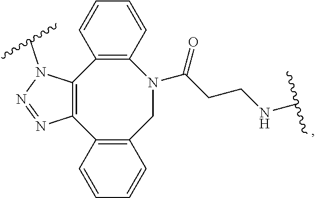 [SMILES: *NCCC(=O)N1Cc2ccccc2-c2nnn(*)c2-c2ccccc21] | 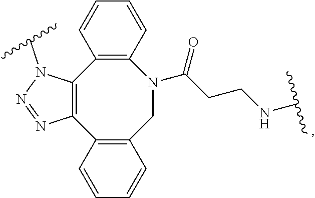 [SMILES: *NCCC(=O)N1Cc2ccccc2-c2nnn(*)c2-c2ccccc21] |
| or | or |
| 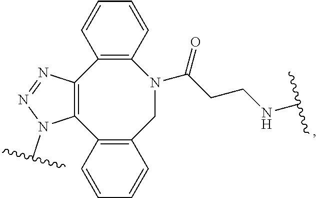 [SMILES: *NCCC(=O)N1Cc2ccccc2-c2c(nnn2*)-c2ccccc21] | 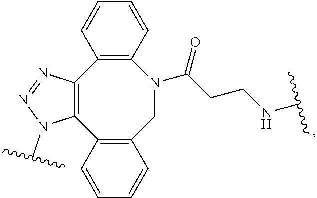 [SMILES: *NCCC(=O)N1Cc2ccccc2-c2c(nnn2*)-c2ccccc21] |
| 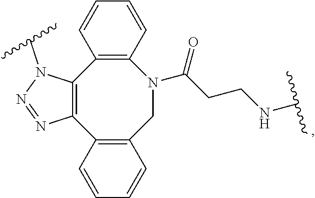 [SMILES: *NCCC(=O)N1Cc2ccccc2-c2nnn(*)c2-c2ccccc21] | 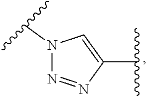 [SMILES: *c1cn(*)nn1] |
| or | or |
| 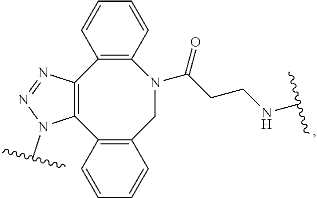 [SMILES: *NCCC(=O)N1Cc2ccccc2-c2c(nnn2*)-c2ccccc21] | 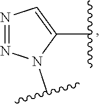 [SMILES: *c1cnnn1*] |
| 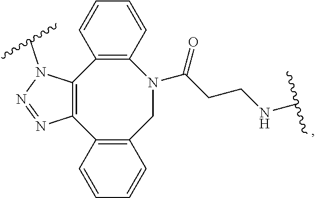 [SMILES: *NCCC(=O)N1Cc2ccccc2-c2nnn(*)c2-c2ccccc21] | 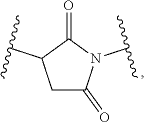 [SMILES: *C1CC(=O)N(*)C1=O] |
| or | |
| 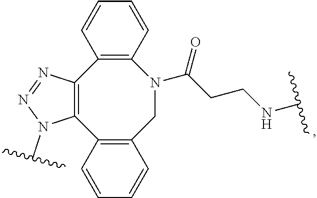 [SMILES: *NCCC(=O)N1Cc2ccccc2-c2c(nnn2*)-c2ccccc21] | |

wherein the

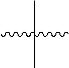

indicates the atom through which the RG¹ or RG² is bonded to the adjacent groups in the formula.

In some embodiments, HG is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, HG is —(CH₂)₁₋₅SO₃H. In another embodiment, HG is —(CH₂)ₙ—NH—(CH₂)₁₋₅SO₃H, wherein n is 1, 2, 3, 4, or 5. In another embodiment, HG is —(CH₂)ₙ C(O)NH—(CH₂)₁₋₅SO₃H, wherein n is 1, 2, 3, 4, or 5. In another embodiment, HG is —(CH₂CH₂O)ₘ—C(O)NH—(CH₂)₁₋₅SO₃H, wherein m is 1, 2, 3, 4, or 5. In another embodiment, HG is —(CH₂)ₙ—N((CH₂)₁₋₅C(O)NH(CH₂)₁₋₅SO₃H)₂, wherein n is 1, 2, 3, 4, or 5. In another embodiment, HG is —(CH₂)ₙ—C(O)N((CH₂)₁₋₅C(O)NH(CH₂)₁₋₅SO₃H)₂, wherein n is 1, 2, 3, 4, or 5. In another embodiment, HG is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, HG is —(CH₂)₁₋₅PO₃H, —(CH₂)ₙ—NH—(CH₂)₁₋₅PO₃H, —(CH₂)ₙ—C(O)NH—(CH₂)₁₋₅PO₃H, —(CH₂CH₂O)ₘ—C(O)NH—(CH₂)₁₋₅PO₃H, —(CH₂)ₙ—N((CH₂)₁₋₅C(O)NH(CH₂)₁₋₅PO₃H)₂, —(CH₂)ₙ—C(O)N((CH₂)₁₋₅C(O)NH(CH₂)₁₋₅PO₃H)₂, or —(CH₂CH₂O)ₘ—C(O)N((CH₂)₁₋₅C(O)NH(CH₂)₁₋₅PO₃H)₂, wherein n is 1, 2, 3, 4, or 5, and m is 1, 2, 3, 4, or 5. In one embodiment, HG is —(CH₂)₁₋₅PO₃H. In another embodiment, HG is —(CH₂)ₙ—NH—(CH₂)₁₋₅PO₃H, wherein n is 1, 2, 3, 4, or 5. In another embodiment, HG is —(CH₂)ₙ C(O)NH—(CH₂)₁₋₅ PO₃H, wherein n is 1, 2, 3, 4, or 5. In another embodiment, HG is —(CH₂CH₂O)ₘ—C(O)NH—(CH₂)₁₋₅PO₃H, wherein m is 1, 2, 3, 4, or 5. In another embodiment, HG is —(CH₂)ₙ—N((CH₂)₁₋₅C(O)NH(CH₂)₁₋₅PO₃H)₂, wherein n is 1, 2, 3, 4, or 5. In another embodiment, HG is —(CH₂)ₙ—C(O)N((CH₂)₁₋₅C(O)NH(CH₂)₁₋₅PO₃H)₂, wherein n is 1, 2, 3, 4, or 5. In another embodiment, HG is —(CH₂CH₂O)ₘ—C(O)N((CH₂)₁₋₅C(O)NH(CH₂)₁₋₅PO₃H)₂, wherein m is 1, 2, 3, 4, or 5.

In some embodiments, HG is —(CH₂)₁₋₅N⁺(R^{M})₃, —(CH₂)ₙ—NH—(CH₂)₁₋₅N⁺(R^{M})₃, —(CH₂)ₙ—C(O)NH—(CH₂)₁₋₅N⁺(R^{M})₃, —(CH₂CH₂O)ₘ—C(O)N—(CH₂)₁₋₅N⁺(R^{M})₃, —(CH₂)ₙ—N((CH₂)₁₋₅C(O)NH(CH₂)₁₋₅N⁺(R^{M})₃)₂, —(CH₂)ₙ—C(O)N((CH₂)₁₋₅C(O)NH(CH₂)₁₋₅N⁺(R^{M})₃)₂, or —(CH₂CH₂O)ₘ—C(O)N((CH₂)₁₋₅C(O)NH(CH₂)₁₋₅N⁺(R^{M})₃)₂, wherein n is 1, 2, 3, 4, or 5, m is 1, 2, 3, 4, or 5, and R^{M}, at each occurrence, is independently H, C₁₋₆alkyl, C₃₋₇cycloalkyl or C₁₋₆alkyl-C₃₋₇cycloalkyl, or, two R^{M} together with the nitrogen atom to which they are attached, form a 3-7-membered heterocycloalkyl ring. In one embodiment, HG is —(CH₂)₁₋₅N⁺(R^{M})₃. In another embodiment, HG is —(CH₂)ₙ—NH—(CH₂)₁₋₅N⁺(R^{M})₃, wherein n is 1, 2, 3, 4, or 5. In another embodiment, HG is —(CH₂)ₙ—C(O)NH—(CH₂)₁₋₅N⁺(R^{M})₃, wherein n is 1, 2, 3, 4, or 5. In another embodiment, HG is —(CH₂CH₂O)ₘ—C(O)NH—(CH₂)₁₋₅N⁺(R^{M})₃, wherein m is 1, 2, 3, 4, or 5. In another embodiment, HG is —(CH₂)ₙ—N((CH₂)₁₋₅C(O)NH(CH₂)₁₋₅N⁺(R^{M})₃)₂, wherein n is 1, 2, 3, 4, or 5. In another embodiment, HG is —(CH₂)ₙ—C(O)N((CH₂)₁₋₅C(O)NH(CH₂)₁₋₅N⁺(R^{M})₃)₂, wherein n is 1, 2, 3, 4, or 5. In another embodiment, HG is —(CH₂CH₂O)ₘ—C(O)N((CH₂)₁₋₅C(O)NH(CH₂)₁₋₅N⁺(R^{M})₃)₂, wherein m is 1, 2, 3, 4, or 5.

In some embodiments, HG is —(CH₂)₁₋₅N⁺Me₃, —(CH₂)ₙ—NH—(CH₂)₁₋₅N⁺Me₃, —(CH₂)ₙ—C(O)NH—(CH₂)₁₋₅N⁺Me₃, —(CH₂CH₂O)ₘ—C(O)NH—(CH₂)₁₋₅N⁺Me₃, —(CH₂)ₙ—N((CH₂)₁₋₅C(O)NH(CH₂)₁₋₅N⁺Me₃)₂, —(CH₂)ₙ—C(O)N((CH₂)₁₋₅C(O)NH(CH₂)₁₋₅N⁺Me₃)₂, or —(CH₂CH₂O)ₘ—C(O)N((CH₂)₁₋₅C(O)NH(CH₂)₁₋₅N⁺Me₃)₂, wherein n is 1, 2, 3, 4, or 5, and m is 1, 2, 3, 4, or 5. In one embodiment, HG is —(CH₂)₁₋₅N⁺Me₃. In another embodiment, HG is —(CH₂)ₙ NH—(CH₂)₁₋₅N⁺Me₃, wherein n is 1, 2, 3, 4, or 5. In another embodiment, HG is —(CH₂)ₙ—C(O)NH—(CH₂)₁₋₅N⁺Me₃, wherein n is 1, 2, 3, 4, or 5. In another embodiment, HG is —(CH₂CH₂O)ₘ—C(O)NH—(CH₂)₁₋₅N⁺Me₃, wherein m is 1, 2, 3, 4, or 5. In another embodiment, HG is —(CH₂)ₙ—N((CH₂)₁₋₅C(O)NH(CH₂)₁₋₅N⁺Me₃)₂, wherein n is 1, 2, 3, 4, or 5. In another embodiment, HG is —(CH₂)ₙ—C(O)N((CH₂)₁₋₅C(O)NH(CH₂)₁₋₅N⁺Me₃)₂, wherein n is 1, 2, 3, 4, or 5. In another embodiment, HG is —(CH₂CH₂O)ₘ—C(O)N((CH₂)₁₋₅C(O)NH(CH₂)₁₋₅N⁺Me₃)₂, wherein m is 1, 2, 3, 4, or 5.

In some embodiments, HG is:

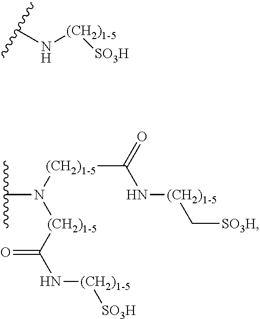 [SMILES: *NCS(=O)(=O)O ; *N(CC(=O)NCCS(=O)(=O)O)CC(=O)NCS(=O)(=O)O]

or salts thereof, wherein the

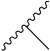

indicates the atom through which the HG is bonded to the adjacent groups in the formula. In one instance HG has one of the following structures:

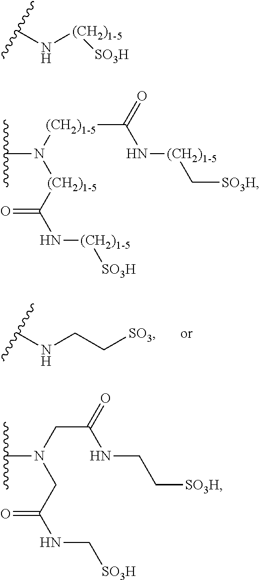

In some embodiments, HG is an amine, or salts thereof, for instance, a quaternary amine, e.g.,

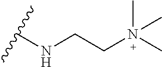

wherein the

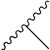

indicates the atom through which the HG is bonded to the adjacent groups in the formula.

In some embodiments, HG is a phosphonic acid, or salts thereof, e.g.,

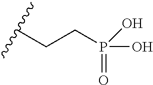 [SMILES: *CCP(=O)(O)O]

wherein the

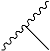

indicates the atom through which the HG is bonded to the adjacent groups in the formula.

In some embodiments, HG is a phosphonic acid, or salts thereof, e.g.,

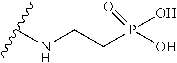 [SMILES: *NCCP(=O)(O)O]

wherein the

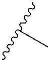

indicates the atom through which the HG is bonded to the adjacent groups in the formula.

In some embodiments, HG is a sugar residue, e.g., (galactose),

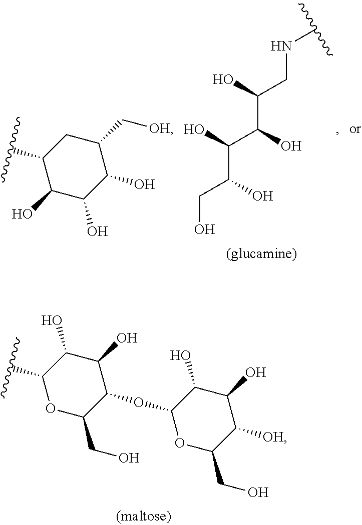

wherein the

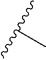

indicates the atom through which the HG is bonded to the adjacent groups in the formula.

In some embodiments, SP¹ and SP² are independently, in each instance, absent, or selected from the group consisting of C₁₋₆ alkylene, —NH—, —C(O)—, (—CH₂—CH₂—O)ₑ, —NH—CH₂—CH₂—(—O—CH₂—CH₂)ₑ—C(O)—, —C(O)—(CH₂)ᵤ—C(O)—, —C(O)—NH—(CH₂)ᵥ—, polyglycine (e.g., ((glycine)₄-sehne)_{f} wherein subscript f is an integer from 1 to 6), and combinations thereof, wherein subscript e is an integer from 0 to 4, subscript u is an integer from 1 to 8, and subscript v is an integer from 1 to 8. In some instances, SP¹ and SP² are independently, in each instance, as shown in Table 6 below.

TABLE 6
SP¹ and SP²

| SP¹ | SP² |
|---|---|
| absent | absent |
| absent | Ch₁₋ [symbol] alkylene, |
| absent | —NH—, |
| absent | —C(O)—, |
| absent | (—CH₂—CH₂—O) [symbol] |
| absent | —NH—CH₂—CH₂—(—O—CH₂—CH₂) [symbol] —C(O)—, |
| absent | —C(O)—(CH₃) [symbol] —C(O)—, |
| absent | —C(O)—NH—(CH₂) [symbol] |
| absent | (glycine)₄-serine |
| C₁₋ [symbol] alkylene, | absent |
| C₁₋ [symbol] alkylene, | C₁₋₈ alkylene, |
| C₁₋ [symbol] alkylene, | —NH—, |
| C₁₋ [symbol] alkylene, | —C(O)—, |
| C₁₋ [symbol] alkylene, | (—CH₂—CH₂—O) [symbol] |
| C₁₋ [symbol] alkylene, | —NH—CH₂—CH₂—(—O—CH₂—CH₂) [symbol] —C(O)—, |
| C₁₋ [symbol] alkylene, | —C(O)—(CH₂) [symbol] —C(O)—, |
| C₁₋ [symbol] alkylene, | —C(O)—NH—(CH₂) [symbol] |
| C₁₋ [symbol] alkylene, | (glycine)₄₋serine |
| NH—, | absent |
| NH—, | C₁₋ [symbol] alkylene, |
| NH—, | —NH—, |
| NH—, | —C(O)—, |
| NH—, | (—CH₂—CH₂—O) [symbol] |
| NH—, | —NH—CH₂—CH₂—(—O—CH₂—CH₂) [symbol] —C(O)—, |
| NH—, | —C(O)—(CH₂) [symbol] —C(O)—, |
| NH—, | —C(O)—NH—(CH₂) [symbol] |
| NH—, | (glycine)₄₋serine |
| NH—, | absent |
| —C(O)—, | C₁₋ [symbol] alkylene, |
| —C(O)—, | —NH—, |
| —C(O)—, | —C(O)—, |
| —C(O)—, | (—CH₂—CH₂—O) [symbol] |
| —C(O)—, | —NH—CH₂—CH₂—(—O—CH₂—CH₂) [symbol] —C(O)—, |
| —C(O)—, | —C(O)—(CH₂) [symbol] —C(O)—, |
| —C(O)—, | —C(O)—NH—(CH₂) [symbol] |
| —C(O)—, | (glycine)₄-serine |
| (—CH₂—CH₂—O) [symbol] | absent |
| (—CH₂—CH₂—O) [symbol] | C₁₋ [symbol] alkylene, |
| (—CH₂—CH₂—O) [symbol] | —NH—, |
| (—CH₂—CH₂—O) [symbol] | —C(O)—, |
| (—CH₂—CH₂—O) [symbol] | (—CH₂—CH₂—O) [symbol] |
| (—CH₂—CH₂—O) [symbol] | —NH—CH₂—CH₂—(—O—CH₂—CH₂) [symbol] C(O)—, |
| (—CH₂—CH₂—O) [symbol] | —C(O)—(CH₂) [symbol] C(O)—, |
| (—CH₂—CH₂—O) [symbol] | —C(O)—NH—(CH₂) [symbol] |
| (—CH₂—CH₂—O) [symbol] | (glycine)₄-serine |
| —NH—CH₂—CH₂—(—O—CH₂—CH₂) [symbol] —C(O)—, | absent |
| —NH—CH₂—CH₂—(—O—CH₂—CH₂) [symbol] —C(O)—, | C₁₋ [symbol] alkylene, |
| —NH—CH₂—CH₂—(—O—CH₂—CH₂) [symbol] —C(O)—, | —NH—, |
| —NH—CH₂—CH₂—(—O—CH₂—CH₂) [symbol] —C(O)—, | —C(O)—, |
| —NH—CH₂—CH₂—(—O—CH₂—CH₂) [symbol] —C(O)—, | (—CH₂—CH₂—O) [symbol] |
| —NH—CH₂—CH₂—(—O—CH₂—CH₂) [symbol] —C(O)—, | —NH—CH₂—CH₂—(—O—CH₂—CH₂) [symbol] —C(O)—, |
| —NH—CH₂—CH₂—(—O—CH₂—CH₂) [symbol] —C(O)—, | —C(O)—(CH₂) [symbol] —C(O)—, |
| —NH—CH₂—CH₂—(—O—CH₂—CH₂) [symbol] —C(O)—, | —C(O)—NH—(CH₂) [symbol] |
| —NH—CH₂—CH₂—(—O—CH₂—CH₂) [symbol] —C(O)—, | (glycine)₄-serine |
| —C(O)—(CH₂) [symbol] —C(O)—, | absent |
| —C(O)—(CH₂) [symbol] —C(O)—, | C₁₋ [symbol] alkylene, |
| —C(O)—(CH₂) [symbol] —C(O)—, | —NH—, |
| —C(O)—(CH₂) [symbol] —C(O)—, | —C(O)—, |
| —C(O)—(CH₂) [symbol] —C(O)—, | (—CH₂—CH₂—O) [symbol] |
| —C(O)—(CH₂) [symbol] —C(O)—, | —NH—CH₂—CH₂—(—O—CH₂—CH₂) [symbol] —C(O)—, |
| —C(O)—(CH₂) [symbol] —C(O)—, | —C(O)—(CH₂) [symbol] —C(O)—, |
| —C(O)—(CH₂) [symbol] —C(O)—, | —C(O)—NH—(CH₂) [symbol] |
| —C(O)—(CH₂) [symbol] —C(O)—, | (glycine)₄-serine |
| —C(O)—NH—(CH₂) [symbol] | absent |
| —C(O)—NH—(CH₂) [symbol] | C₁₋ [symbol] alkylene, |
| —C(O)—NH—(CH₂) [symbol] | —NH—, |
| —C(O)—NH—(CH₂) [symbol] | —C(O)—, |
| —C(O)—NH—(CH₂) [symbol] | (—CH₂—CH₂—O) [symbol] |
| —C(O)—NH—(CH₂) [symbol] | —NH—CH₂—CH₂—(—O—CH₂—CH₂ [symbol] —C(O)—, |
| —C(O)—NH—(CH₂) [symbol] | —C(O)—(CH₂) [symbol] —C(O)—, |
| —C(O)—NH—(CH₂) [symbol] | —C(O)—NH—(CH₂) [symbol] |
| —C(O)—NH—(CH₂) [symbol] | (glycine)₄-serine |

[symbol]

indicates data missing or illegible when filed

Any combination of a row from Table R and a row from Table S may be present in a linker of the disclosure.

In some embodiments, the linker is selected from the group consisting of:

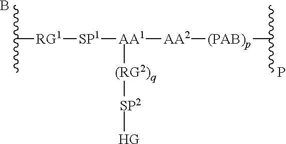

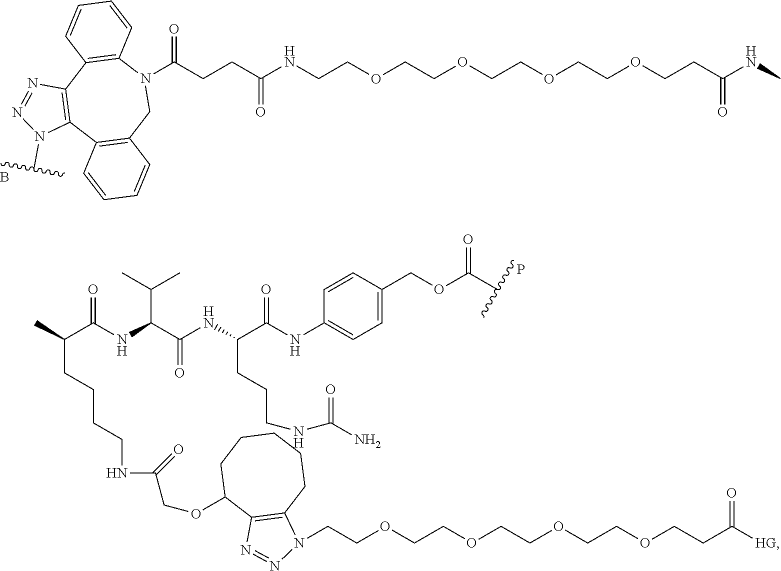

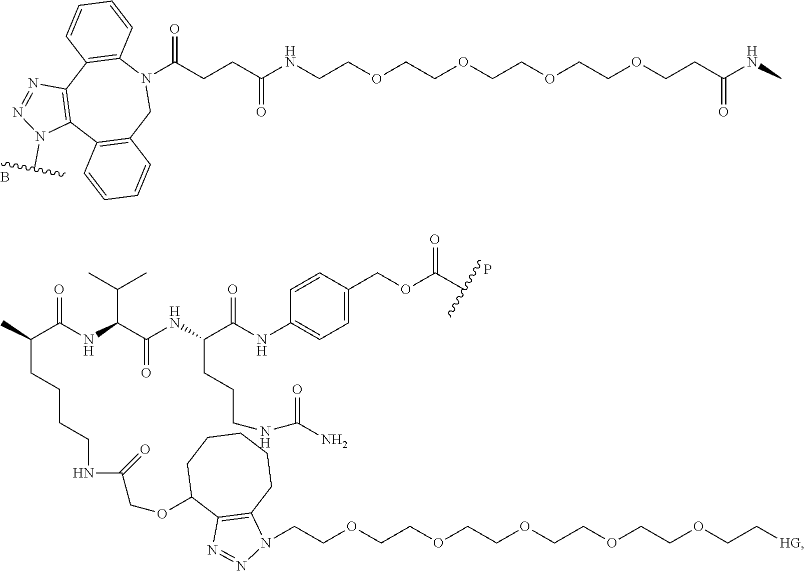

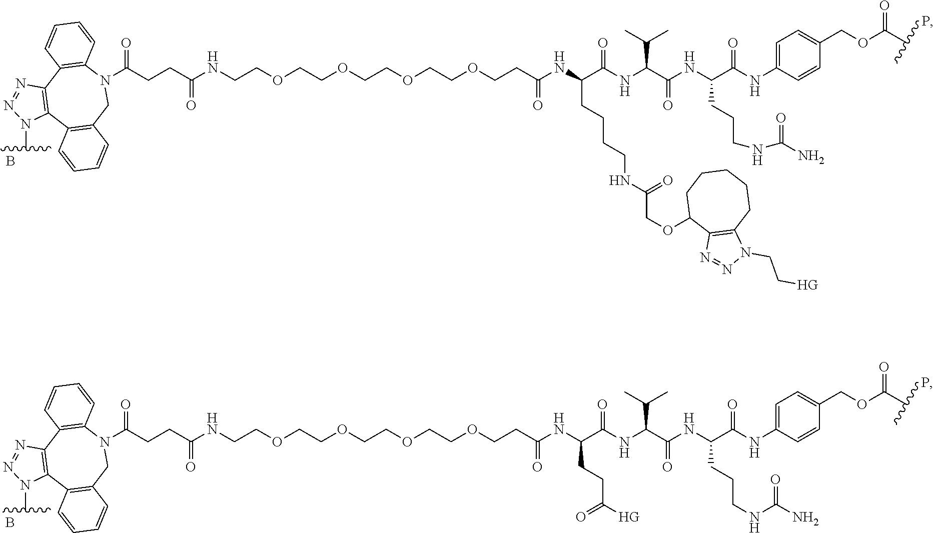

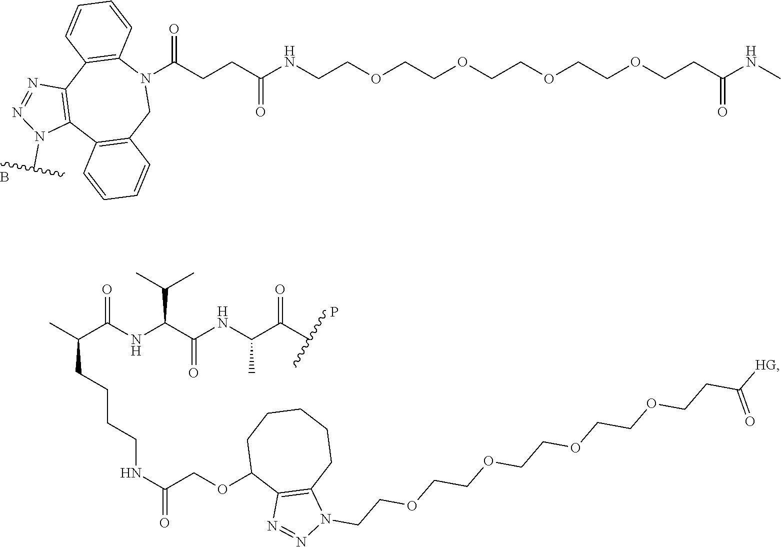

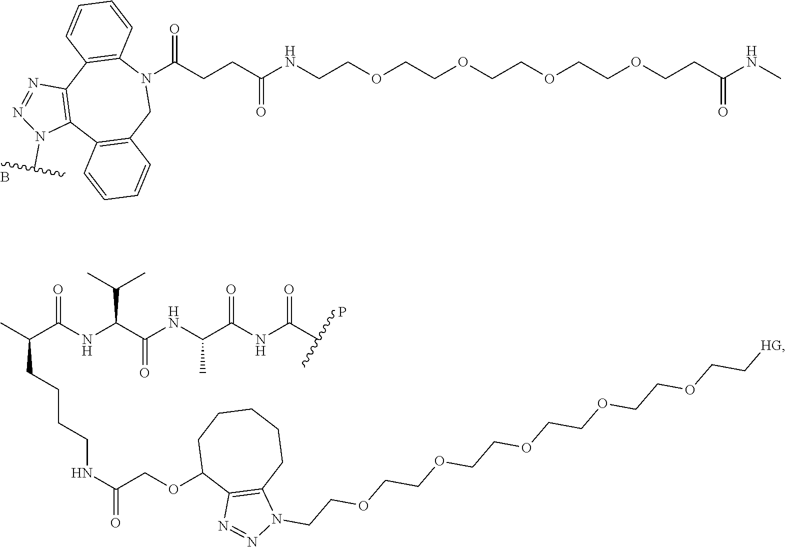

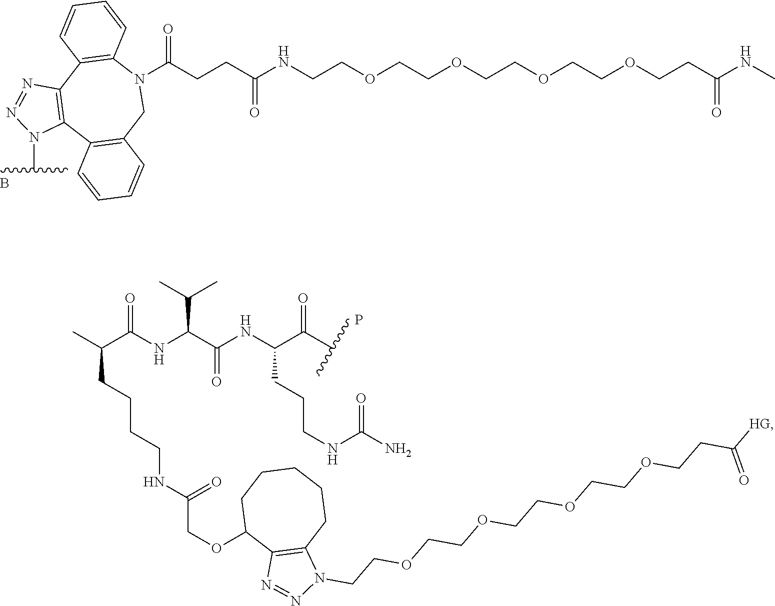

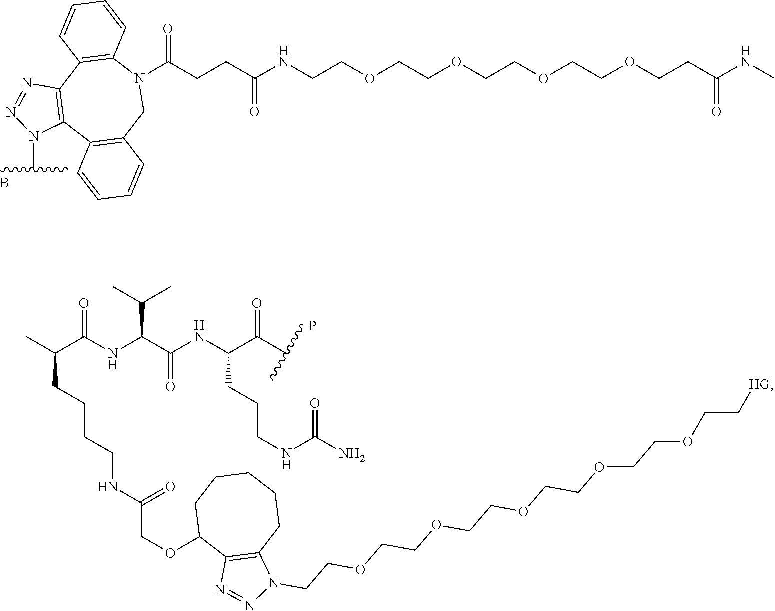

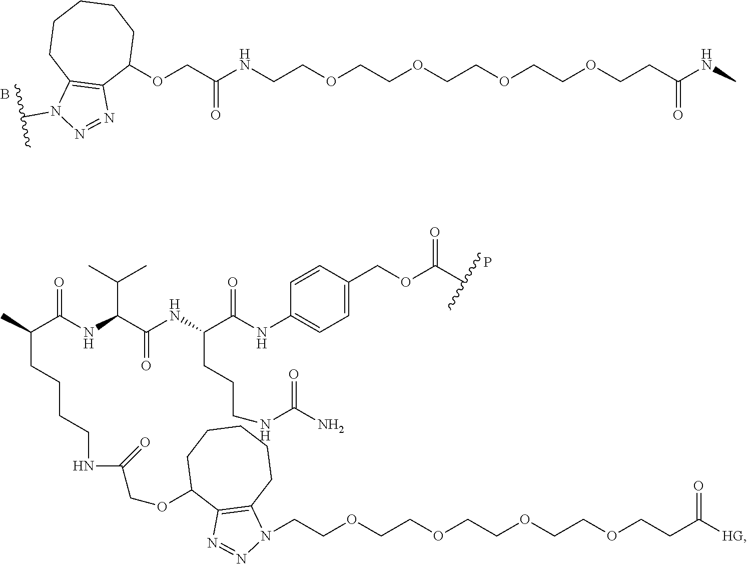

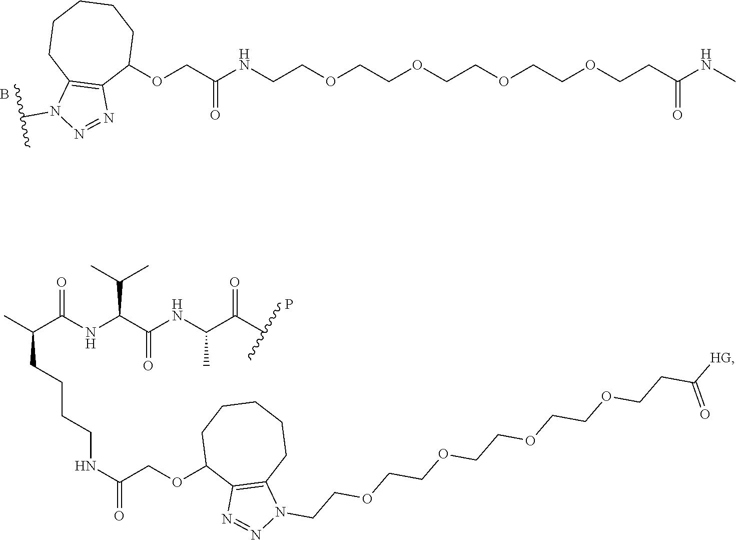

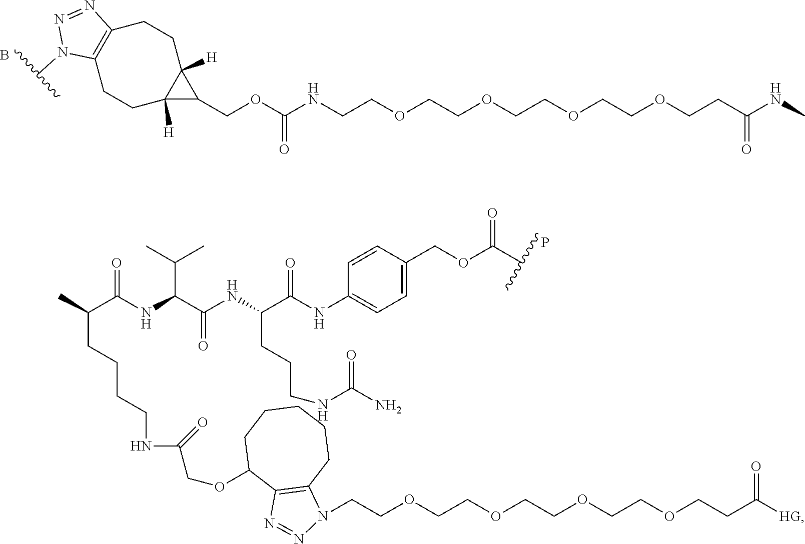

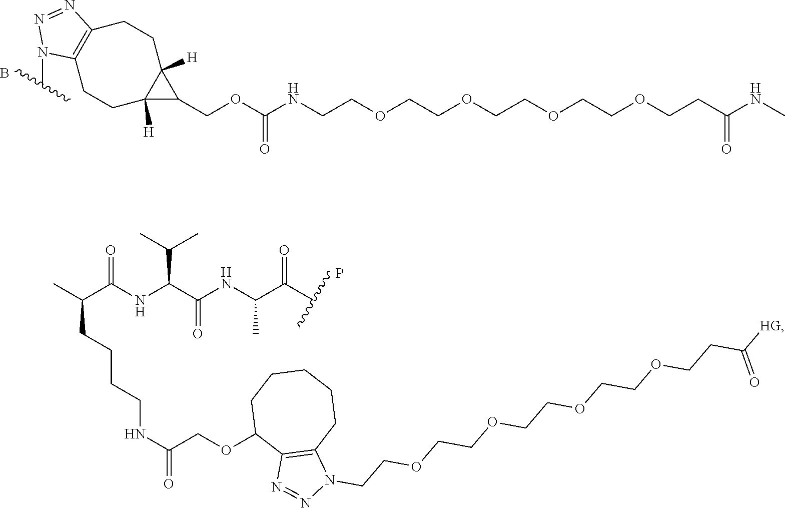

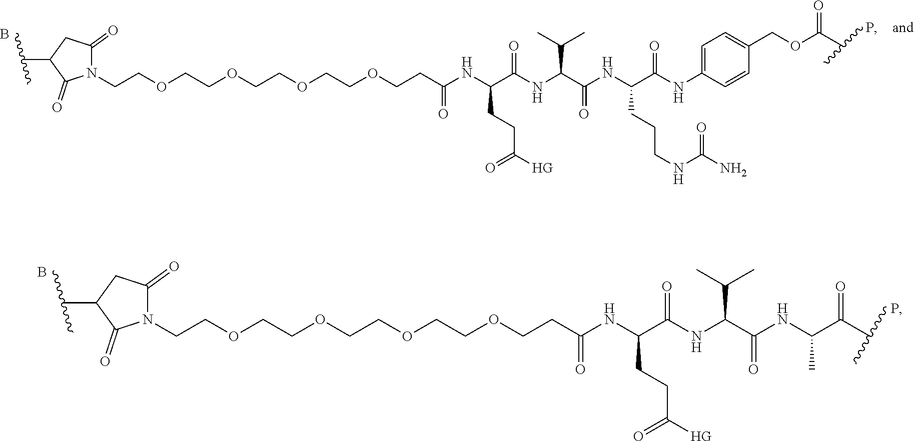

or a stereoisomeric form thereof, or a regioisomer thereof, or a mixture of regioisomers thereof, wherein each

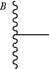

is a bond to a binding protein of this disclosure; and each

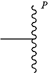

is a bond to a payload (i.e., a GR agonist).

In some embodiments, the linker is selected from the group consisting of:

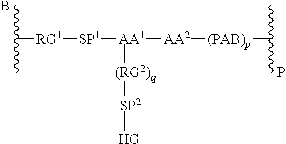

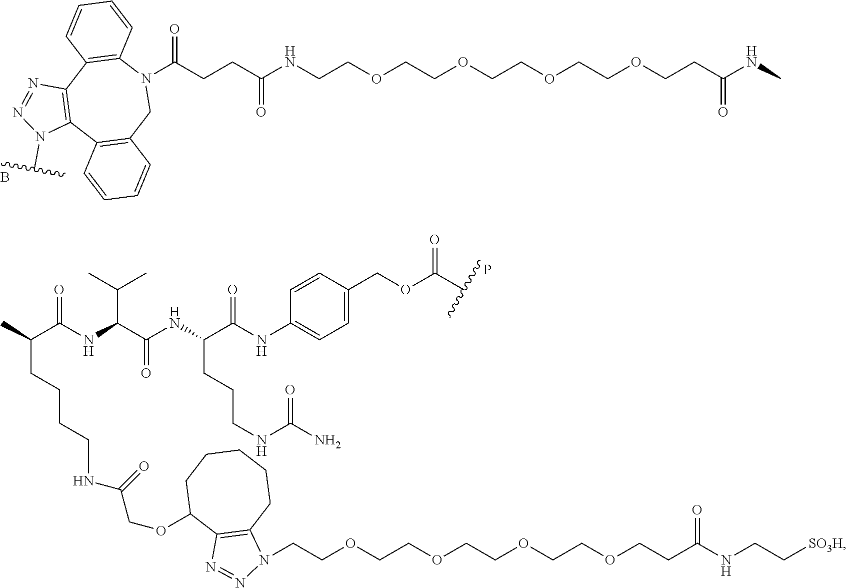

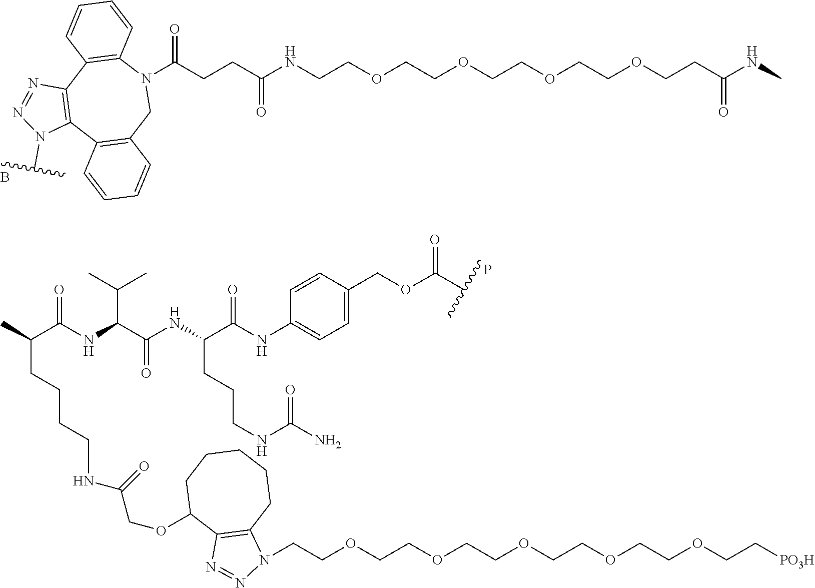

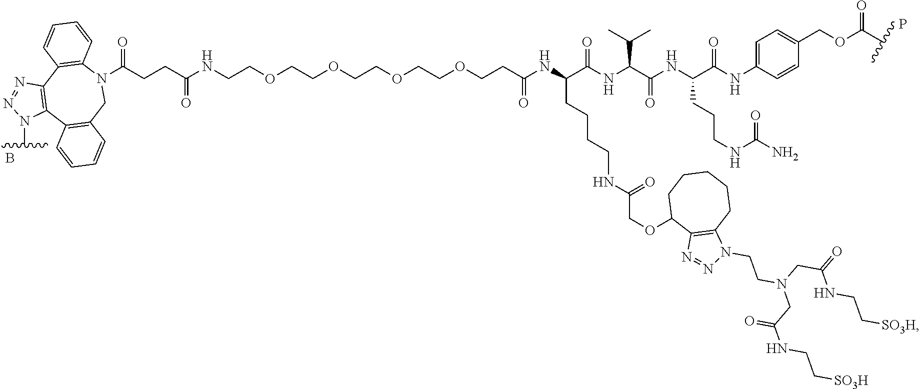

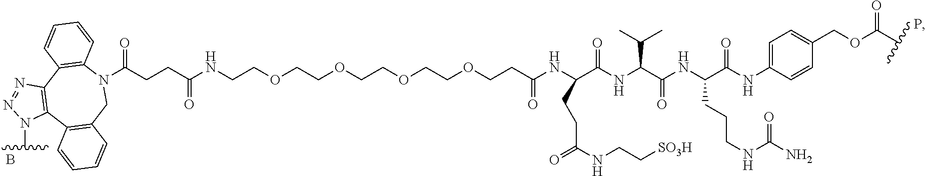

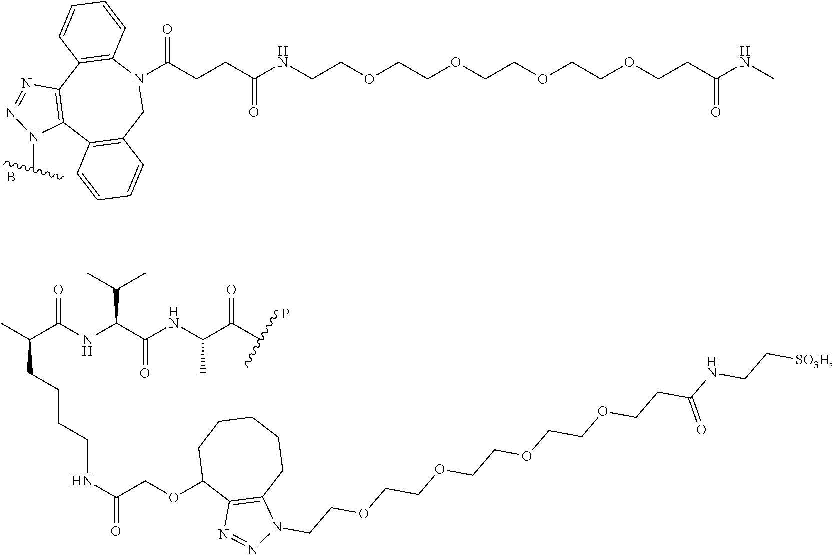

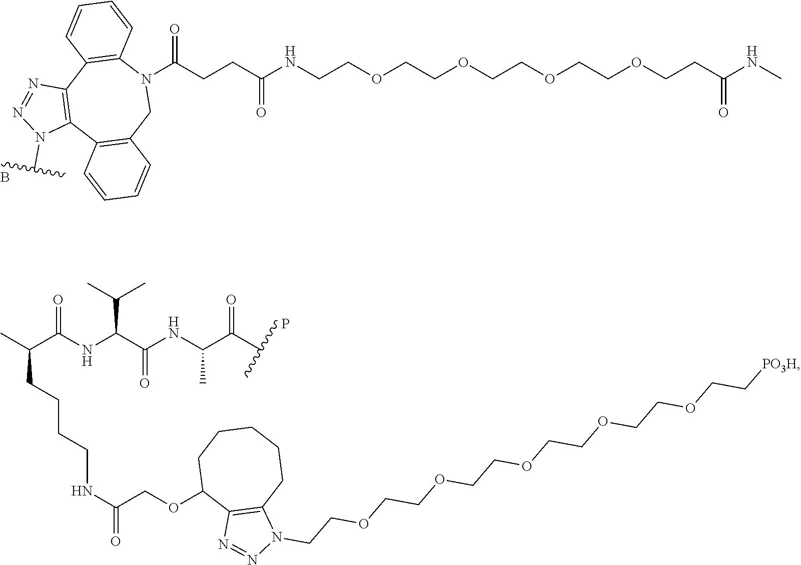

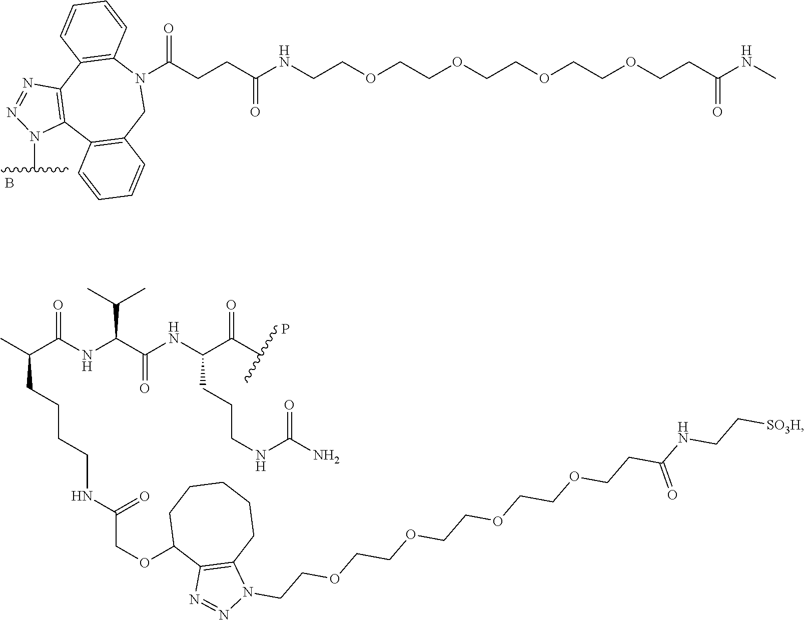

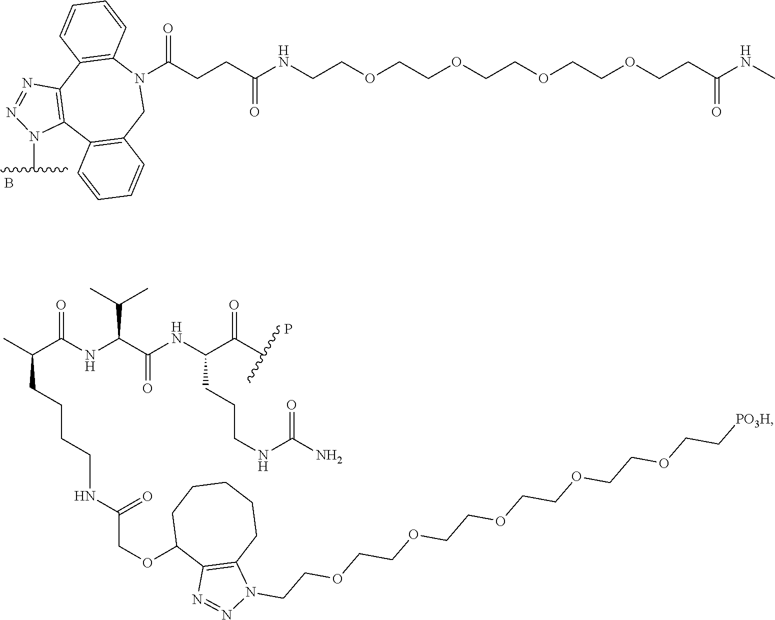

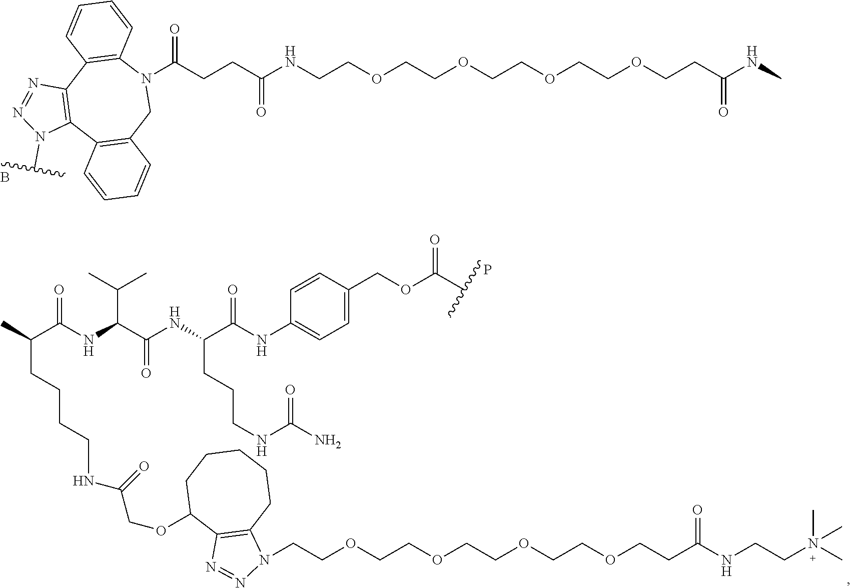

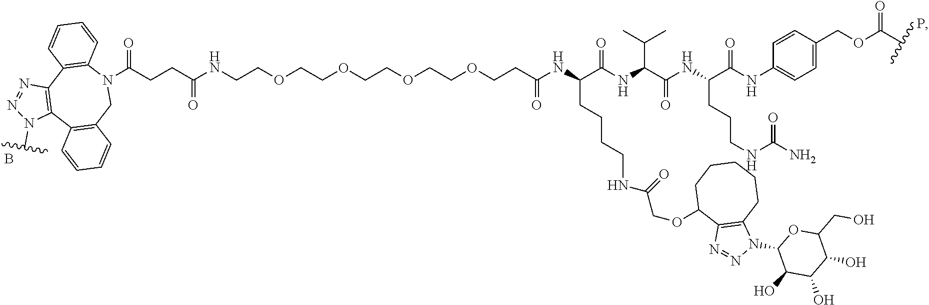

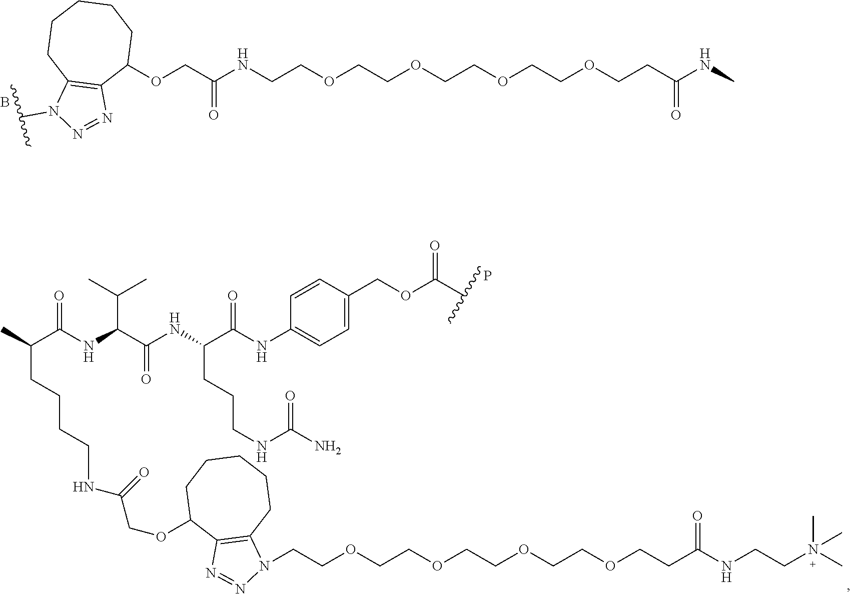

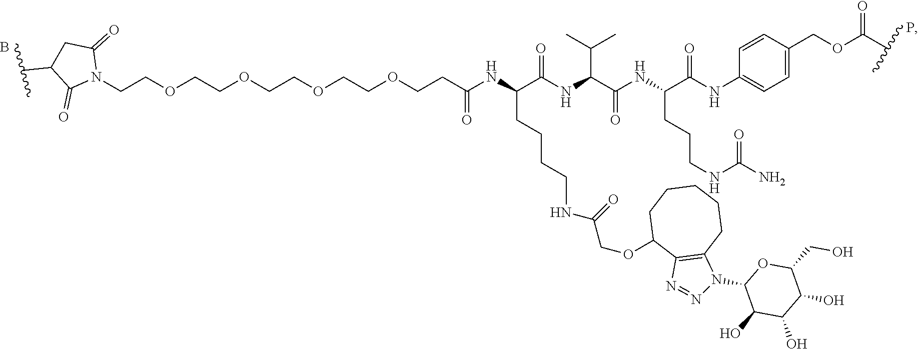

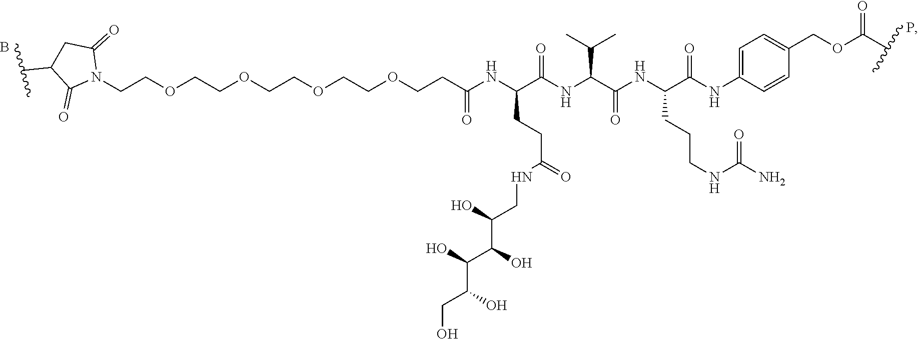

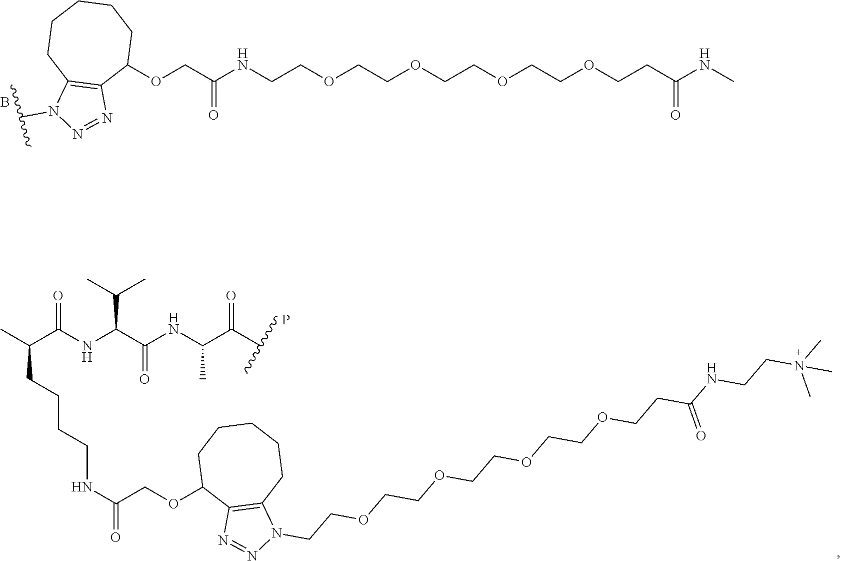

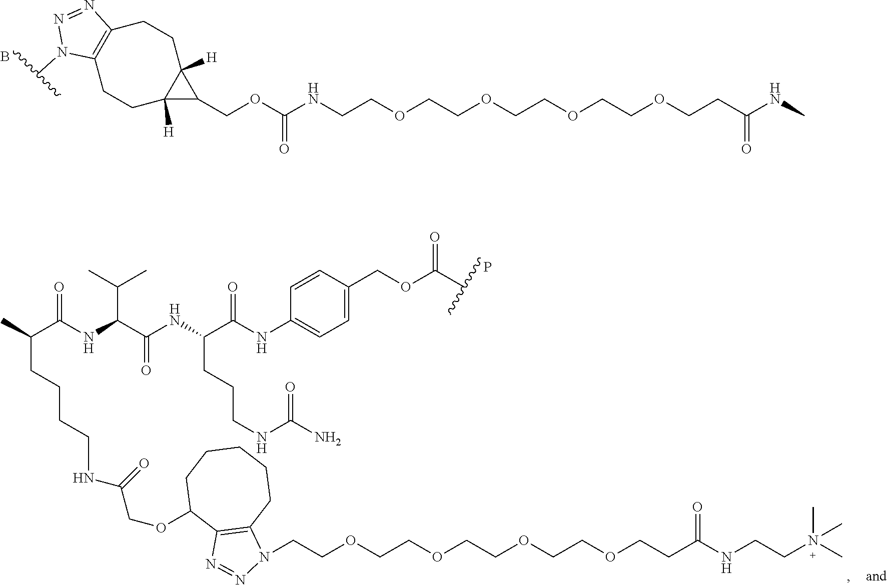

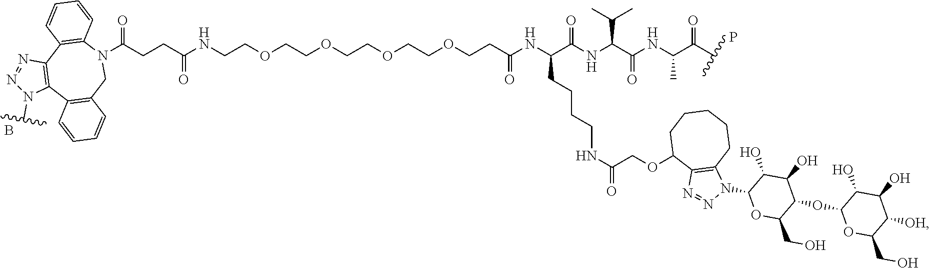

or a stereoisomeric form thereof, or a regioisomer thereof, or a mixture of regioisomers thereof, wherein each

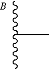

is a bond to a binding protein of this disclosure; and each

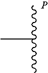

is a bond to the payload (i.e., a GR agonist).

In some embodiments, in the structure

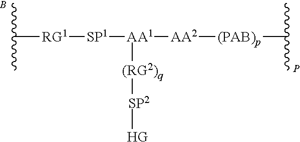

or a stereoisomeric form thereof, or a regioisomer thereof, or a mixture of regioisomers thereof, RG¹ is

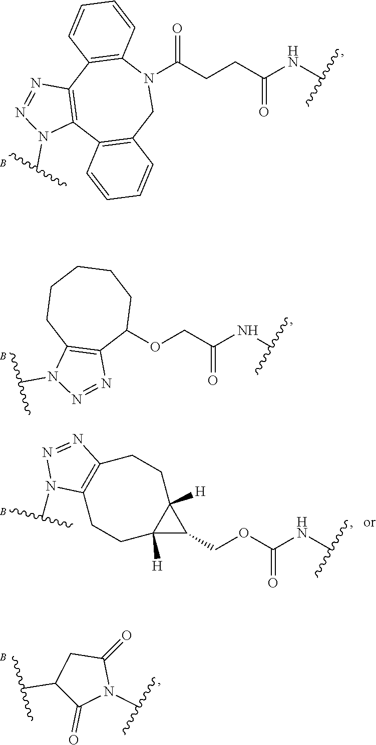

### HG is

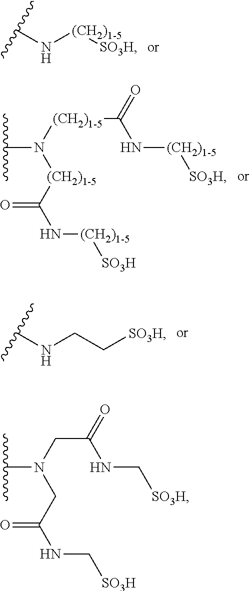

wherein each

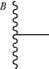

is a bond to a binding protein of this disclosure, each

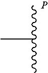

is a bond to the payload (i.e., a GR agonist); and each

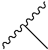

indicates the atom through which the group is attached to the rest of the molecule.

In some embodiments, the linker comprises the following structure:

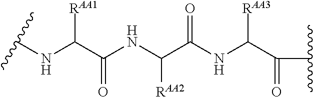

wherein R^{AA1}, R^{AA2}, and R^{AA3} are each, independently, amino acid side chains, at least one of which is bonded directly or indirectly to -(RG²)_{q}-SP²-HG

In some embodiments, R^{AA1} is a lysine, glutamine, glutamic acid, or aspartic acid side chain bonded directly or indirectly to HG, and R^{AA2} and R^{AA3} are either valine and alanine or valine and citrulline sidechains respectively.

In some embodiments, AA² is

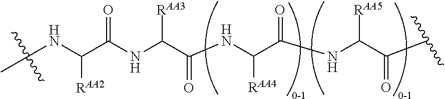

wherein R^{AA2}, R^{AA3}, R^{AA4}, and R^{AA5} are each, independently, amino acid side chains, at least one of which is bonded directly or indirectly to -(RG²)_{q}-SP²-HG, wherein the

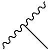

indicates the atom through which AA² is bonded to the adjacent groups in the formula. In some examples, R^{AA2}, R^{AA3}, R^{AA4}, and R^{AA5} are independently in each instance, an amino acid side chain selected from the sidechains of alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or a combination thereof.

In some embodiments, AA² is

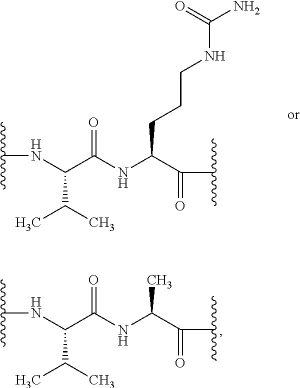 [SMILES: *N[C@H](C(=O)N[C@@H](CCCNC(N)=O)C(*)=O)C(C)C ; *N[C@H](C(=O)N[C@@H](C)C(*)=O)C(C)C]

wherein the

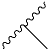

indicates the atom through which AA¹ is bonded to the adjacent groups in the formula.

In one instance AA² is

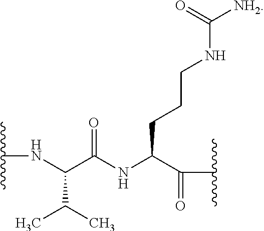 [SMILES: *N[C@H](C(=O)N[C@@H](CCCNC(N)=O)C(*)=O)C(C)C]

In another instance, AA² is

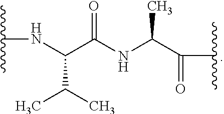 [SMILES: *N[C@H](C(=O)N[C@@H](C)C(*)=O)C(C)C]

In some embodiments, the linker further comprises one or more polyethylene glycol units. For example, in some embodiments, the linker further comprises the following structure:

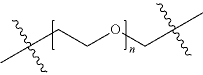

wherein:
n is an integer greater than 1. In some embodiments, n is 1, 2, 3, 4, 5, 6, 7, or 8. In some embodiments, polyethylene glycol units are bound to RG¹.

10. Category X Linkers

Exemplary linkers are described in some detail in, for example: PCT publication No. 2020/146541, the contents of which is incorporated by reference herein in its entirety.

In some specific embodiments, the linker has the following structure:

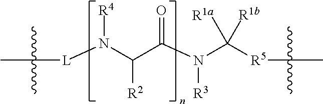

wherein:
L is a linker;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 3-, 4-, 5-, 6-, 7-, or 8-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 3-, 4-, 5-, 6-, 7-, or 8-membered heterocyclyl;
R⁴ is hydrogen or alkyl;
R⁵ is oxygen, NR⁶, or sulfur;
R⁶ is hydrogen or alkyl; and
n is zero, one, two, three, four, five, or six.

In some embodiments, L is conjugated to a binding protein of this disclosure via a click chemistry residue, an amide residue, and a residue comprising two cysteine residues of the polypeptide that are chemically bonded to L.

In some embodiments, R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁴ is hydrogen or alkyl;
R⁵ is oxygen, NR⁶, or sulfur;
R⁶ is hydrogen or alkyl; and
n is zero, one, two, three, four, five, or six.

In some more specific embodiments, the linker has the following structure:

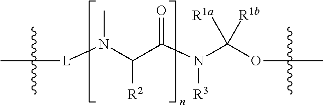

wherein:
L is a linker;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R¹ is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl; and
n is zero, one, two, three, four, live, or six.

In some more specific embodiments, the linker has the following structure:

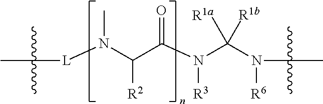

wherein:
L is a linker;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁶ is hydrogen or alkyl; and
n is zero, one, two, three, four, or five.

In some embodiments, the linker has the following structure:

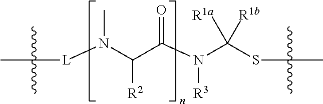

wherein:
L is a linker;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; and
n is zero, one, two, three, four, or five.

In certain more specific embodiments, the linker has the following structure:

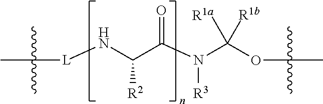

wherein:
L is a linker;
R^{1a} and R^{1b} are, independently, hydrogen, alkyl, alkoxy, alkenyl, alkynyl, aryl, arylalkyl, heteroaryl, or alkylene, wherein when R^{1a} is alkylene, the alkylene is further bonded to R³ to form a 4-, 5-, or 6-membered heterocyclyl;
R² is hydrogen or an amino acid side chain;
R³ is hydrogen, alkyl, or alkylene, wherein when R³ is alkylene, the alkylene is further bonded to R^{1a} to form the 4-, 5-, or 6-membered heterocyclyl; and
n is zero, one, two, three, four, five, or six.

In some embodiments, the linker has the following structure:

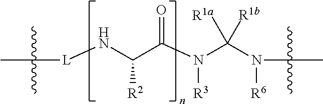

wherein:
L is a linker;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl;
R⁶ is hydrogen or alkyl; and
n is zero, one, two, three, four, or five.

In some more specific embodiments, the linker has the following structure:

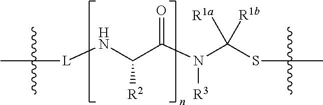

wherein:
L is a linker;
R^{1a} and R^{1b} are, independently, hydrogen, alkyl, alkoxy, alkenyl, alkynyl, aryl, arylalkyl, heteroaryl, alkylene, or heteroalkylene, wherein when R^{1a} is alkylene or heteroalkylene, the alkylene or heteroalkylene is further bonded to R³ to form a 4-, 5-, or 6-membered heterocyclyl;
R² is hydrogen, alkylene, heteroalkylene, or an amino acid side chain, wherein when R² is alkylene or heteroalkylene, the alkylene or heteroalkylene is further bonded to R³ to form a 4-, 5-, or 6-membered heterocyclyl;
R³ is hydrogen, alkyl, alkylene, or heteroalkylene, wherein when R³ is alkylene or heteroalkylene, the alkylene or heteroalkylene is further bonded to R^{1a} or R² to form the 4-, 5-, or 6-membered heterocyclyl; and
n is zero, one, two, three, four, or five.

In certain specific embodiments, the linker has the following structure:

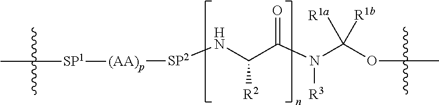

wherein:
R^{1a}, R^{1b}, R², R³, and n are as described in any of the embodiments disclosed herein, and wherein SP¹ and SP², when present, are spacer groups wherein SP¹ further comprises a moiety reactive with a binding protein of this disclosure;
each AA is an amino acid; and
p is an integer from 1 to 10.

In some embodiments, the linker comprises the following structure:

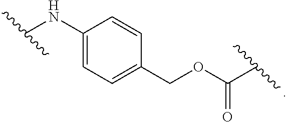 [SMILES: *Nc1ccc(COC(*)=O)cc1]

In some embodiments, the linker further comprises a moiety formed via a reaction with a binding protein of this disclosure that has been modified with a primary amine compound according to the Formula H2N-LL-X, wherein LL is a divalent linker selected from the group consisting of a divalent polyethylene glycol (PEG) group; —(CH₂)ₙ—; —(CH₂CH₂O)ₙ—(CH₂)ₚ—; —(CH₂)ₙ—N(H)C(O)—(CH₂)ₘ—; —(CH₂CH₂O)ₙ—N(H)C(O)—(CH₂CH₂O)ₘ—(CH₂)ₚ—; —(CH₂)ₙ—C(O)N(H)—(CH₂)ₘ—; —(CH₂CH₂O)ₙ—C(O)N(H)—(CH₂CH₂O)ₘ—(CH₂)ₚ—; —(CH₂)ₙ—N(H)C(O)—(CH₂CH₂O)ₘ—(CH₂)ₚ—; —(CH₂CH₂O)ₙ—N(H)C(O)—(CH₂)ₘ—; —(CH₂)ₙ—C(O)N(H)—(CH₂CH₂O)ₘ—(CH₂)ₚ—; and —(CH₂CH₂O)ₙ—C(O)N(H)—(CH₂)ₘ—,
wherein
n is an integer selected from 1 to 12;
m is an integer selected from 0 to 12;
p is an integer selected from 0 to 2; and
X is selected from the group consisting of —SH, —N₃, —C═CH, —C(O)H, tetrazole,

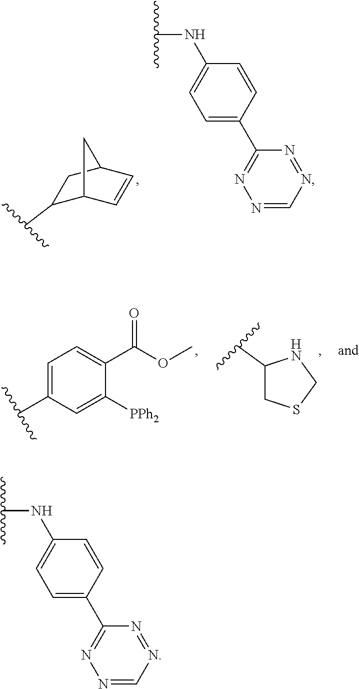

In the above, any of the alkyl or alkylene (i.e., —CH₂—) groups can optionally be substituted, for example with C₁₋₈ alkyl, methylformyl, or —SO₃H. In some embodiments, the alkyl groups are unsubstituted.

In certain embodiments, the primary amine compound is selected from the group consisting of:

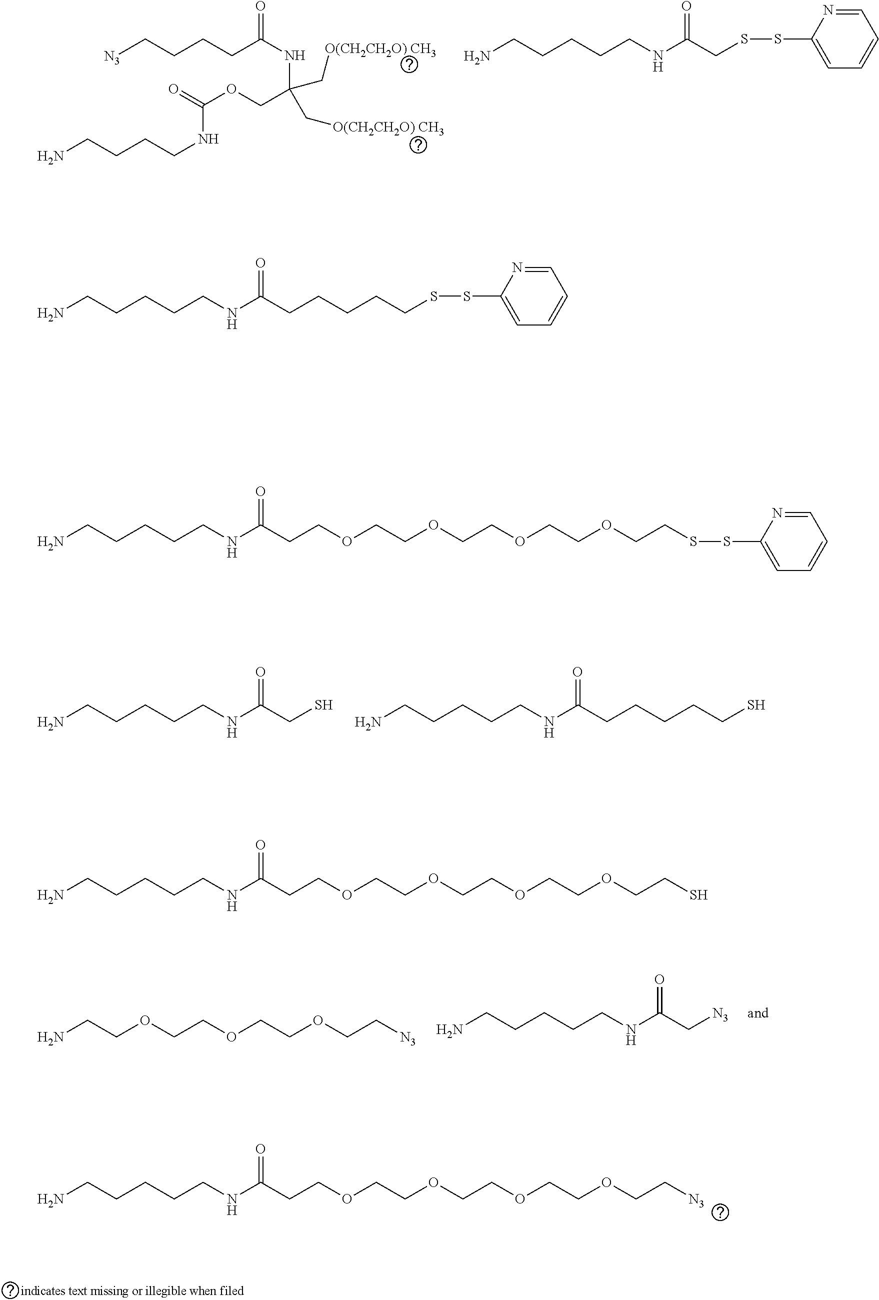

In particular embodiments, the primary amine compound is

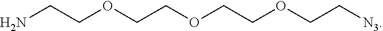 [SMILES: [N-]=[N+]=NCCOCCOCCOCCN]

In some embodiments, the linker of the disclosure is a moiety, for instance a divalent moiety, that covalently links a binding protein of this disclosure to a GR agonist of the disclosure. In other instances, the linker s a trivalent or multivalent moiety that covalently links a binding protein of this disclosure to a GR agonist of the disclosure. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference.

In some embodiments, the linkers are stable in physiological conditions. In some embodiments, the linkers are cleavable, for instance, able to release at least the GR agonist in the presence of an enzyme or at a particular pH range or value. In some embodiments, a linker comprises an enzyme-cleavable moiety. Illustrative enzyme-cleavable moieties include peptide bonds, ester linkages, hydrazones, and disulfide linkages. In some embodiments, the linker comprises a cathepsin-cleavable linker. In some embodiments, the linker comprises a moiety that is stable at certain pHs and cleavable to release the payload portion at other pHs. For instance, in some embodiments, the linker is stable at physiological pH and capable of releasing the payload portion at a local pH in the vicinity of a target.

In some embodiments, the linker comprises a non-cleavable moiety. In some embodiments, the non-cleavable linker is derived from maleimide. In some embodiments, the non-cleavable linkers are derived from an ester. In some embodiments, the non-cleavable linker is derived from an N-hydroxysuccinimide ester. In some embodiments, the non-cleavable linker is derived from

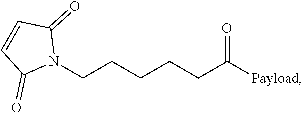

or a residue thereof (e.g., wherein “payload” is a GR agonist). In some embodiments, the non-cleavable linker-payload residue is

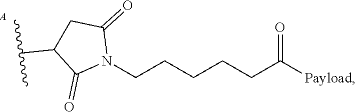

or a regioisomer thereof (e.g., wherein “payload” is a GR agonist).

In some embodiments, the non-cleavable linker is derived from

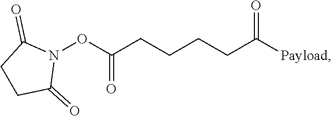

or a residue thereof (e.g., wherein “payload” is a GR agonist).

In some embodiments, the non-cleavable linker-payload residue is

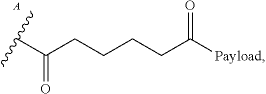

(e.g., wherein “payload” is a GR agonist).

In some embodiments, the linker is maleimide cyclohexane carboxylate or 4-(N-maleimidomethyl)cyclohexanecarboxylic acid (MCC), where the payload (e.g., wherein “payload” is a GR agonist) can be added to either end of the MCC linker. In another embodiment, the linker is

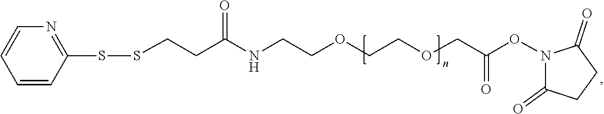

where the payload (e.g., wherein “payload” is a GR agonist) can be added to either end of this linker. In certain embodiments, the linker is a self-stabilizing maleimide:

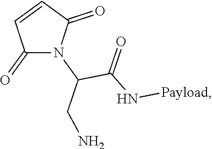

(e.g., wherein “payload” is a GR agonist)

In one exemplary embodiment, the self-stabilizing maleimide linker is where the bond from the amide nitrogen to the payload (e.g., wherein “payload” is a GR agonist) can be a direct bond to the payload; or the bond from the amide nitrogen to the payload (e.g., wherein “payload” is a GR agonist), as shown, contemplates the remainder of the linker. In another exemplary embodiment, the self-stabilizing linker manifests as

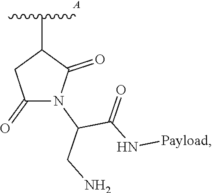

where the bond from the amide nitrogen to the payload can be a direct bond to the payload (e.g., wherein “payload” is a GR agonist of Structure (I) or (II)); or the bond from the amide nitrogen to the payload, as shown, contemplates the remainder of the linker. Without being bound by any particular theory, the self-stabilizing linker includes moieties that stabilize the bond from the self-stabilizing linker to a binding protein of this disclosure. For example, in some embodiments, when the bond from a polypeptide comprising a binding domain (e.g., a fusion protein, or an antibody or an antigen-binding fragment thereof) to a self-stabilizing linker is a carbon-sulfur bond (e.g., following a Michael addition of a polypeptide cysteine to the self-stabilizing maleimide linker), the self-stabilizing linker mitigates retro-Michael additions. More specifically, in the self-stabilizing maleimide (or succinimide) linkers shown, the aminomethyl functionality facilitates rapid hydrolysis of the succinimide Michael addition product to provide

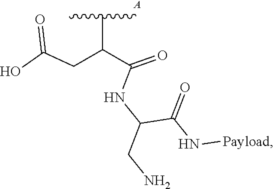

where the bond from the amide nitrogen to the payload (e.g., wherein “payload” is a GR agonist) can be a direct bond to the payload (e.g., wherein “payload” is a GR agonist of Structure (I) or (II)); or the bond from the amide nitrogen to the payload (e.g., wherein “payload” is a GR agonist of Structure (I) or (II)), as shown, contemplates the remainder of the linker; thus, decreasing susceptibility to retro-Michael additions. Moieties other than aminomethyl within self-stabilizing maleimide linkers that stabilize conjugates will be appreciated by those of skill in the art. In the structures, indicates a bond to a binding protein of this disclosure.

In the structures,

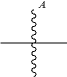

in some examples, indicates a click chemistry residue which results from the reaction of, for example, a polypeptide having an azide or alkyne functionality and a linker-payload having a complementary alkyne or azide functionality. In the structures, in other examples,

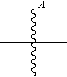

indicates a divalent sulfide which results from the reaction of, for example, one or more polypeptide cysteines with one or more linkers or linker-payloads having maleimide functionality via Michael addition reactions. In the structures, in other examples, indicates an amide bond which results from the reaction of, for example, one or more polypeptide lysines with one or more linkers or linker-payloads having activated or inactivated carboxyl functionality, as would be appreciated by a person of skill in the art.

In one embodiment,

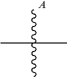

indicates an amide bond which results from the reaction of, for example, one or more polypeptide lysines with one or more linkers or linker-payloads having activated carboxyl functionality, as would be appreciated by a person of skill in the art.

In some embodiments, suitable linkers include those that are chemically bonded to two cysteine residues of a polypeptide, e.g., an antibody. Such linkers can serve to mimic the polypeptide's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L- or D-α-amino acids. In some embodiments, the linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combination thereof (e.g., dipeptides, tripeptides, oligopeptides, polypeptides, and the like). In some embodiments, one or more side chains of the amino acids are linked to a side chain group, described below. In some embodiments, the linker is a peptide comprising or consisting of the amino acids valine and citrulline (e.g., divalent -Val-Cit- or divalent -VCit-). In some embodiments, the linker is a peptide comprising or consisting of the amino acids alanine and alanine, or divalent -AA-. In some embodiments, the linker is a peptide comprising or consisting of the amino acids glutamic acid and alanine, or -EA-. In some embodiments, the linker is a peptide comprising or consisting of the amino acids glutamic acid and glycine, or -EG-. In some embodiments, the linker is a peptide comprising or consisting of the amino acids glycine and glycine, or -GG-. In some embodiments, the linker is a peptide comprising or consisting of the amino acids glutamine, valine, and citrulline, or -Q-V-Cit- or -QVCit-. In some embodiments, the linker is a peptide comprising or consisting of the amino acids glutamic acid, valine, and citrulline, or -E-V-Cit- or -EVCit-. In some embodiments, the linker is a peptide comprising or consisting of the amino acids -GGGGS-. In some embodiments, the linker is a peptide comprising or consisting of the amino acids -GGGGG-. In some embodiments, the linker is a peptide comprising or consisting of the amino acids -GGGGK-. In some embodiments, the linker is a peptide comprising or consisting of the amino acids -GFGG-. In some embodiments, the linker is a peptide comprising or consisting of the amino acids lysine, valine, and citrulline, or -KVCit-. In some embodiments, the linker is a peptide comprising or consisting of the amino acids -KVK-. In some embodiments, the linker is a peptide comprising or consisting of the amino acids -VA-. In any of the embodiments in this paragraph, and throughout this disclosure, the standard three-letter or one-letter amino acid designations are used, as would be appreciated by a person of skill in the art. Exemplary single-letter amino acid designations include G for glycine, K for lysine, S for serine, V for valine, A for alanine, and F for phenylalanine.

In some embodiments, the linker comprises a self-immolative group. The self-immolative group can be any such group known to those of skill. In particular embodiments, the self-immolative group is p-aminobenzyl (PAB), or a derivative thereof. Useful derivatives include p-aminobenzyloxycarbonyl (PABC). Those of skill will recognize that a self-immolative group is capable of carrying out a chemical reaction which releases the remaining atoms of a linker from a payload (e.g., wherein “payload” is a GR agonist of Structure (I) or (II)).

In some embodiments, the linker is

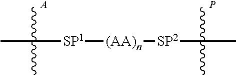

wherein:
   SP¹ is a spacer;
   SP² is a spacer;

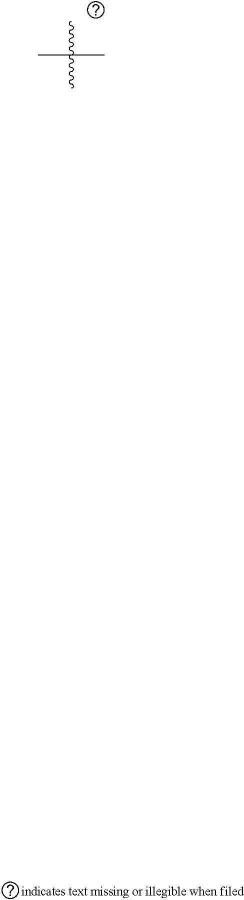

is one or more bonds to a binding protein of this disclosure;

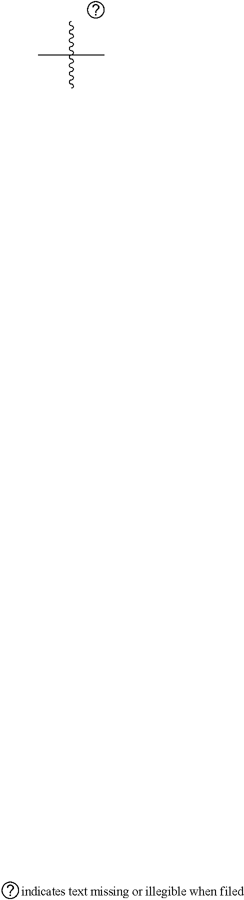

is one or more bonds to a GR agonist;
each AA is an amino acid residue; and
n is an integer from 0 to 10.

The SP¹ spacer is a moiety that connects the (AA)ₙ moiety or residue to a binding protein of this disclosure or to a reactive group residue which is bonded to the polypeptide. Suitable SP¹ spacers include those comprising alkylene or polyether, or both. The ends of the spacers, for example, the portion of the spacer bonded to the polypeptide or an AA, can be moieties derived from reactive moieties that are used for purposes of coupling the polypeptide or an AA to the spacer during chemical synthesis of the conjugate. In some embodiments, n is 1, 2, 3, or 4. In particular embodiments, n is 2. In particular embodiments, n is 3. In particular embodiments, n is 4. In some embodiments, when n is zero, then (AA)ₙ is a bond.

In some embodiments, the SP¹ spacer comprises an alkylene. In some embodiments, the SP¹ spacer comprises a C₅₋₇ alkylene. In some embodiments, the SP¹ spacer comprises a polyether. In some embodiments, the SP¹ spacer comprises a polymer of ethylene oxide such as polyethylene glycol.

In some embodiments, the SP¹ spacer is:

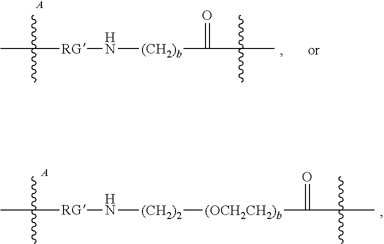

wherein:
RG¹ is a reactive group residue following reaction of a reactive group RG with a binding protein of this disclosure;
is a bond to the polypeptide;

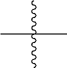

is a bond to (AA)ₙ wherein n is an integer from 0 to 10; and
b is an integer from 2 to 8.

The reactive group RG can be any reactive group known to those of skill in the art to be capable of forming one or more bonds to the polypeptide. The reactive group RG is a moiety comprising a portion in its structure that is capable of reacting with a binding protein of this disclosure, e.g., reacting with a polypeptide at its cysteine or lysine residues, or at an azide moiety, for example, a PEG-N₃ functionalized antibody at one or more glutamine residues. Following conjugation to the polypeptide, the reactive group becomes the reactive group residue (RG′). Illustrative reactive groups include those that comprise haloacetyl, isothiocyanate, succinimide, N-hydroxysuccinimide, or maleimide portions that are capable of reacting with a binding protein of this disclosure.

In some embodiments, reactive groups include alkynes. In some embodiments, the alkynes are alkynes capable of undergoing 1,3-cycloaddition reactions with azides in the absence of copper catalysts, such as strained alkynes. Strained alkynes are suitable for strain-promoted alkyne-azide cycloadditions (SPAAC), and include cycloalkynes, for example, cyclooctynes and benzannulated alkynes. Suitable alkynes include, moieties having one of the following structures:

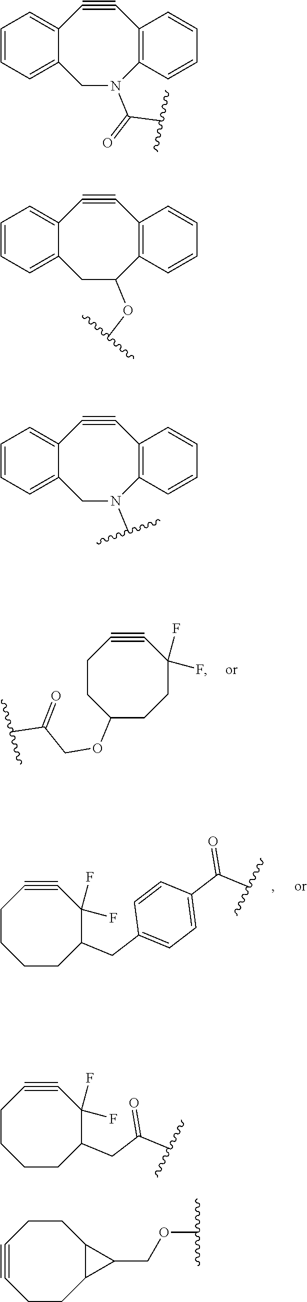

In more specific embodiments, moieties include one of the following structures:

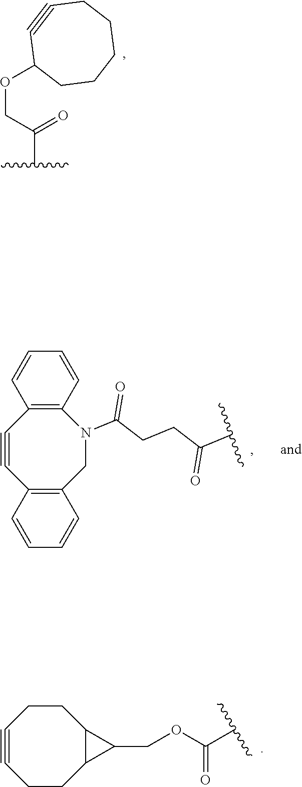 [SMILES: *C(=O)COC1C#CCCCCC1 ; *C(=O)CCC(=O)N1Cc2ccccc2C#Cc2ccccc21 ; *C(=O)OCC1C2CCC#CCCC21]

In some embodiments, a binding protein of this disclosure is bonded directly to RG′. In some embodiments, a binding protein of this disclosure is bonded to RG′ via a spacer, for instance SP⁴, located between and RG′. In particular embodiments, a binding protein of this disclosure is bonded indirectly to RG′ via SP⁴,

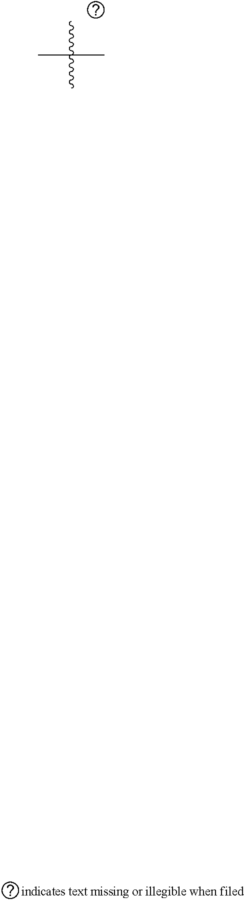

for example, a PEG spacer. As discussed in detail below, in some embodiments, a binding protein of this disclosure is prepared by functionalizing with one or more azido groups. Each azido group can react with RG to form RG′. In particular embodiments, a binding protein of this disclosure is derivatized with -PEG-N₃ linked to a glutamine residue. Exemplary —N₃ derivatized polypeptides, methods for their preparation, and methods for their use in reacting with RG are provided herein. In some embodiments, RG is an alkyne suitable for participation in 1,3-cycloadditions, and RG′ is a regioisomeric 1,2,3-triazolyl moiety formed from the reaction of RG with an azido-functionalized polypeptide. By way of further example, in some embodiments, RG′ is linked to a binding protein of this disclosure as shown in or a mixture of each regioisomer, for example the following structures:

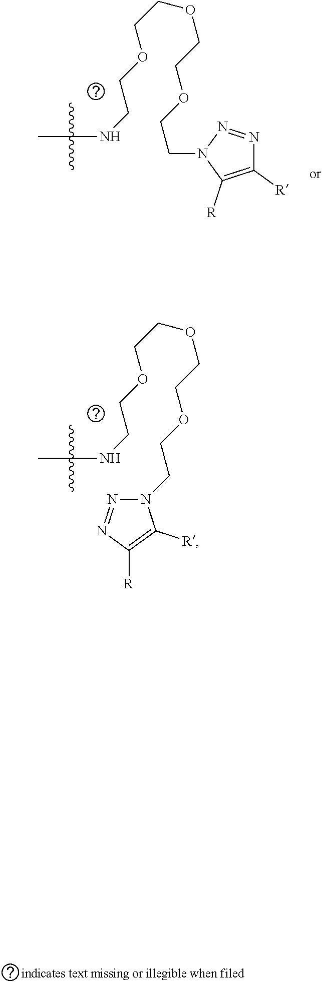

wherein each R and R′ is as described or exemplified herein.

The SP² spacer, when present, is a moiety that connects the (AA)ₙ moiety to the payload (i.e., a GR agonist). Suitable spacers include those described above as SP¹ spacers. Further suitable SP² spacers include those comprising alkylene or polyether, or both. The ends of the SP² spacers, for example, the portion of the spacer directly bonded to the payload (i.e., a GR agonist) or an AA, can be moieties derived from reactive moieties that are used for purposes of coupling the payload (e.g., wherein “payload” is a GR agonist of Structure (I) or (II)) or AA to the SP² spacer during the chemical synthesis of the conjugate. In some examples, the ends of the SP² spacers, for example, the portion of the SP² spacer directly bonded to the payload (e.g., wherein “payload” is a GR agonist of Structure (I) or (II)) or an AA, can be residues of reactive moieties that are used for purposes of coupling the payload (e.g., wherein “payload” is a GR agonist of Structure (I) or (II)) or an AA to the spacer during the chemical synthesis of the conjugate.

In some embodiments, the SP² spacer, when present, is selected from the group consisting of —NH-(p-C₆H₄)-CH2-, —NH-(p-C₆H₄)—CH₂OC(O)—, an amino acid, a dipeptide, a tripeptide, an oligopeptide, —O—, —N(H)—

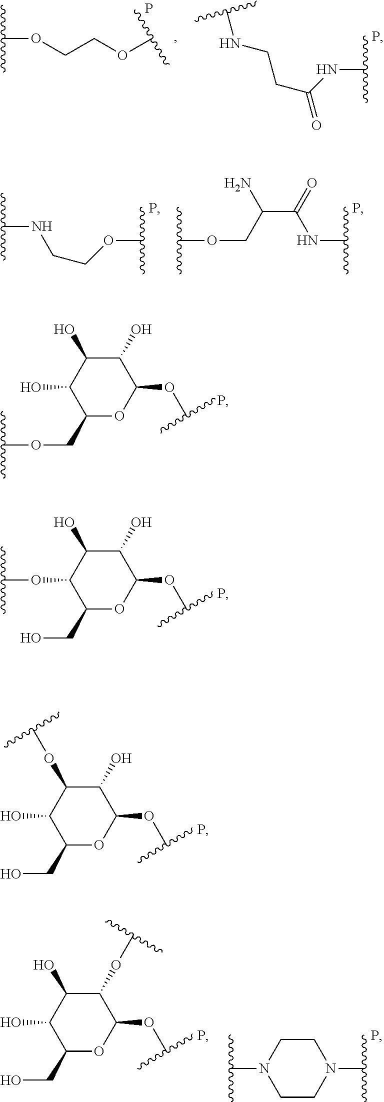

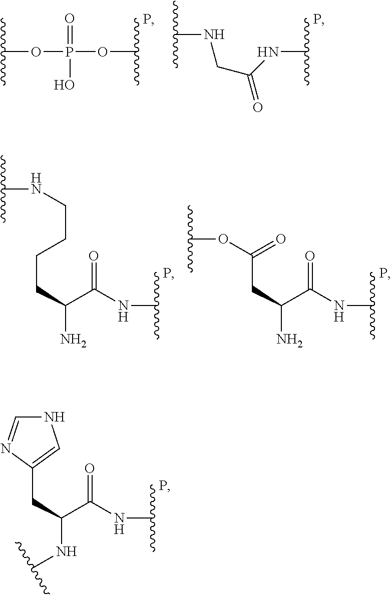

and any combinations thereof. In some embodiments, each

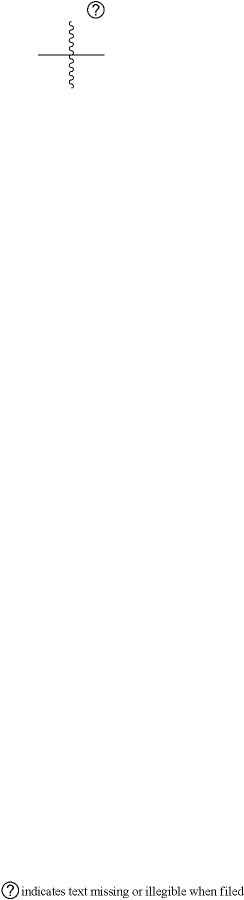

is a bond to the payload (e.g., wherein “payload” is a GR agonist of Structure (I) or (II)), and each

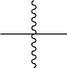

is a bond to (AA)ₙ.

In the above formulae, each (AA)ₙ is an amino acid or, optionally, a p-aminobenzyloxycarbonyl residue (PABC),

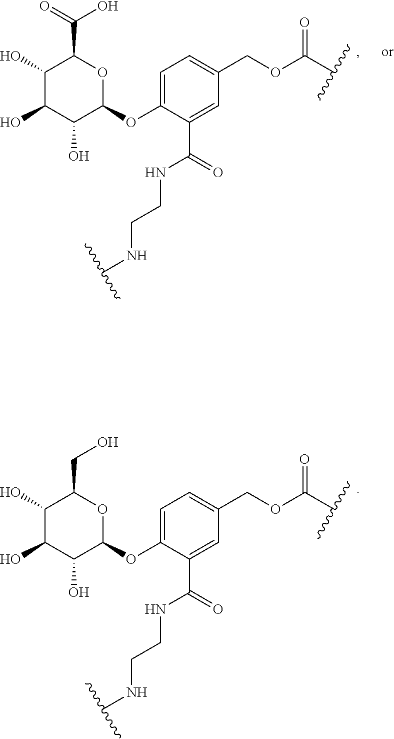 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

If PABC is present, in some embodiments, then only one PABC is present. In some embodiments, the PABC residue, if present, is bonded to a terminal AA in the (AA)ₙ group, proximal to the payload (e.g., wherein “payload” is a GR agonist of Structure (I) or (II)). If

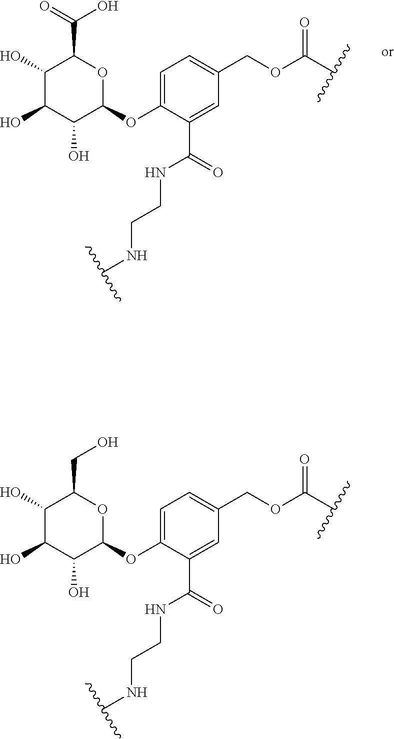 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

are present, then only

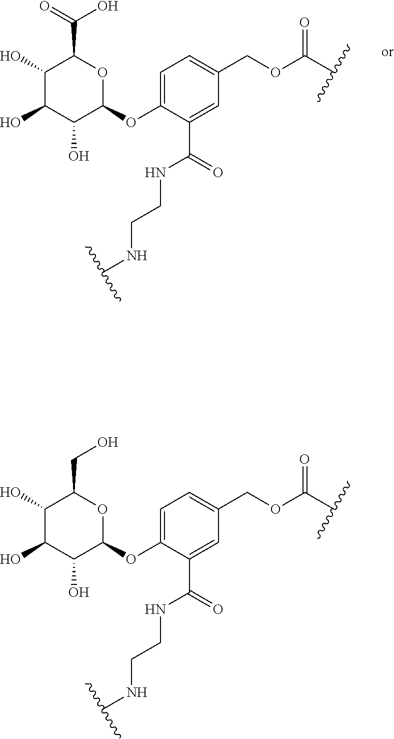 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

is present.

In some embodiments, the

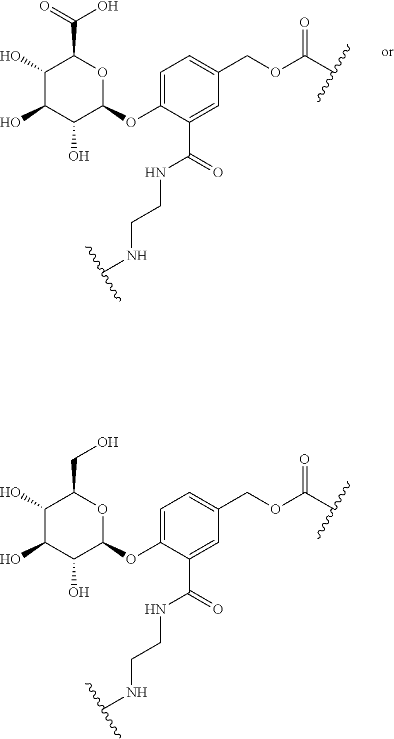 [SMILES: *NCCNC(=O)c1cc(COC(*)=O)ccc1O[C@@H]1O[C@H](C(=O)O)[C@@H](O)[C@H](O)[C@H]1O ; *NCCNC(=O)c1cc(COC(*)=O)ccc1O[C@@H]1O[C@H](CO)[C@@H](O)[C@H](O)[C@H]1O]

residues, if present, are bonded to the payload (e.g., wherein “payload” is a GR agonist of Structure (I) or (II)) via the benzyloxycarbonyl moiety, and no AA is present. Suitable amino acids for each AA include natural, non-natural, standard, non-standard, proteinogenic, non-proteinogenic, and L- or D-α-amino acids. In some embodiments, the AA comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or any combinations thereof (e.g., dipeptides, tripeptides, and oligopeptides, and the like). In some embodiments, one or more side chains of the amino acids is linked to a side chain group, described below. In some embodiments, n is two. In some embodiments, the (AA)ₙ is valine-citrulline. In some embodiments, (AA)ₙ is citrulline-valine. In some embodiments, (AA)ₙ is valine-alanine. In some embodiments, (AA)ₙ is alanine-valine. In some embodiments, (AA)ₙ is valine-glycine. In some embodiments, (AA)ₙ is glycine-valine. In some embodiments, n is three. In some embodiments, the (AA)ₙ is valine-citrulline-PABC. In some embodiments, (AA)ₙ is citrulline-valine-PABC. In some embodiments, (AA)ₙ is glutamate-valine-citrulline. In some embodiments, (AA)ₙ is glutamine-valine-citrulline. In some embodiments, (AA)ₙ is lysine-valine-alanine. In some embodiments, (AA)ₙ is lysine-valine-citrulline. In some embodiments, n is four. In some embodiments, (AA)ₙ is glutamate-valine-citrulline-PABC.

In some embodiments, (AA)ₙ is glutamine-valine-citrulline-PABC. Those of skill will recognize PABC as a residue of p-aminobenzyloxycarbonyl with the following structure:

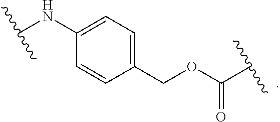 [SMILES: *Nc1ccc(COC(*)=O)cc1]

The PABC residue has been shown to facilitate cleavage of certain linkers in vitro and in vivo. Those of skill will recognize PAB as a divalent residue of p-aminobenzyl or —NH-(p-C₆H₄)—CH₂—.

In some embodiments, the linker is:

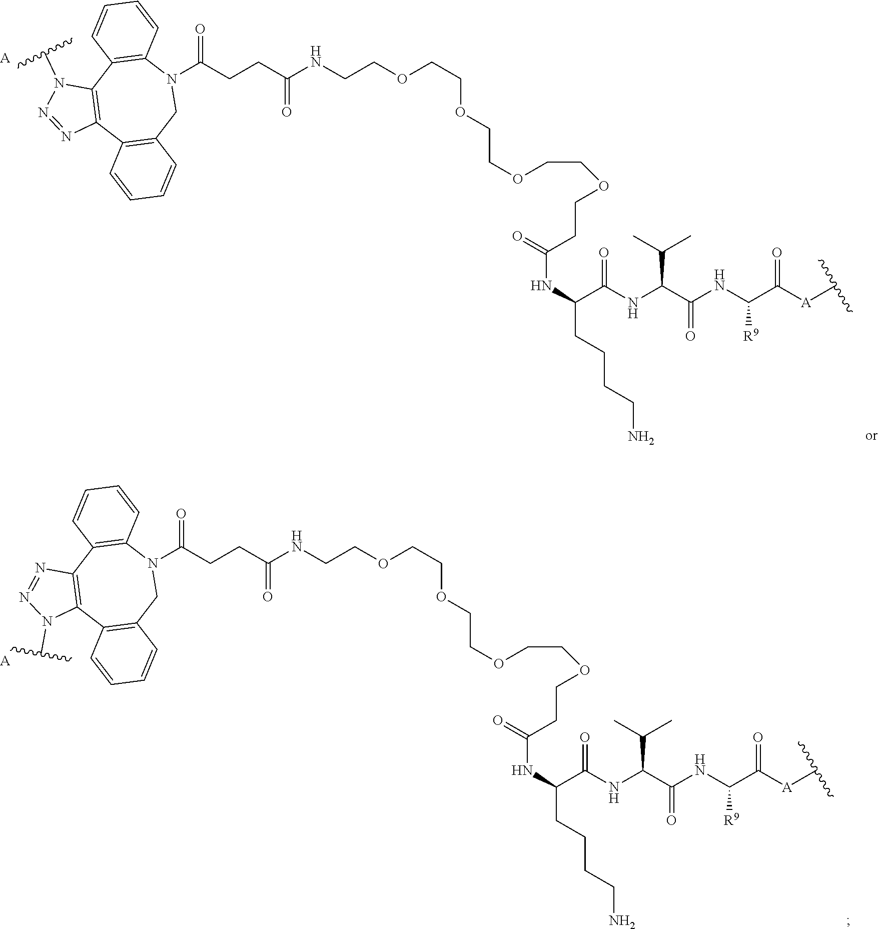

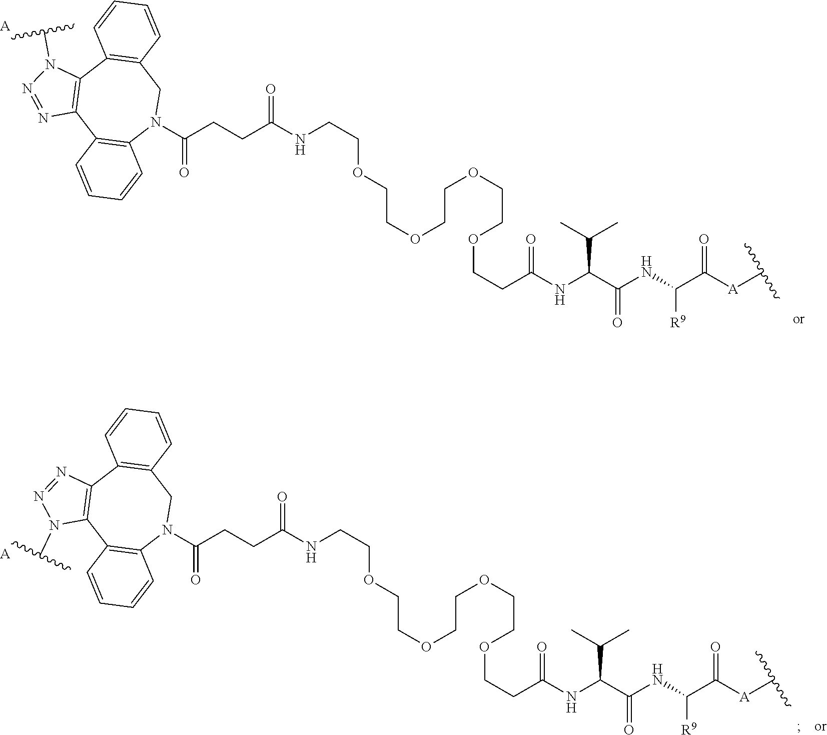

wherein:

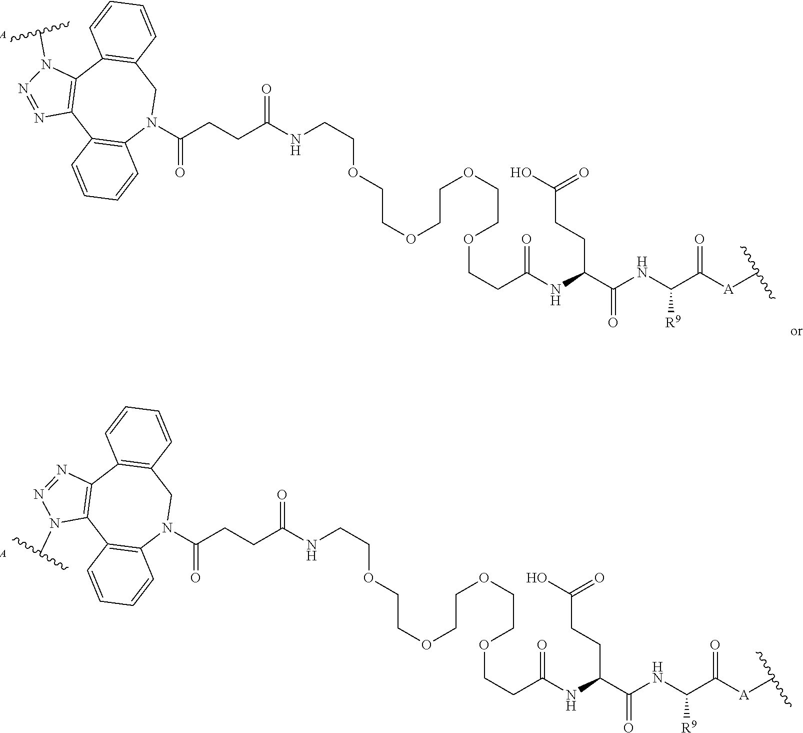

each

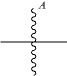

is a bond to a binding protein of this disclosure;
each

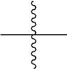

is a bond to the payload (e.g., wherein “payload” is a GR agonist of Structure (I) or (II));
each R⁹ is —CH₃ or —(CH₂)₃N(H)C(O)NH₂; and
each A is —O—, —N(H)—,

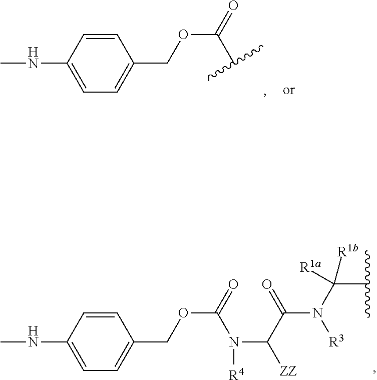

where ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to a binding protein of this disclosure is via a PEG spacer to a glutamine residue of the polypeptide.

In some embodiments, the linker is:

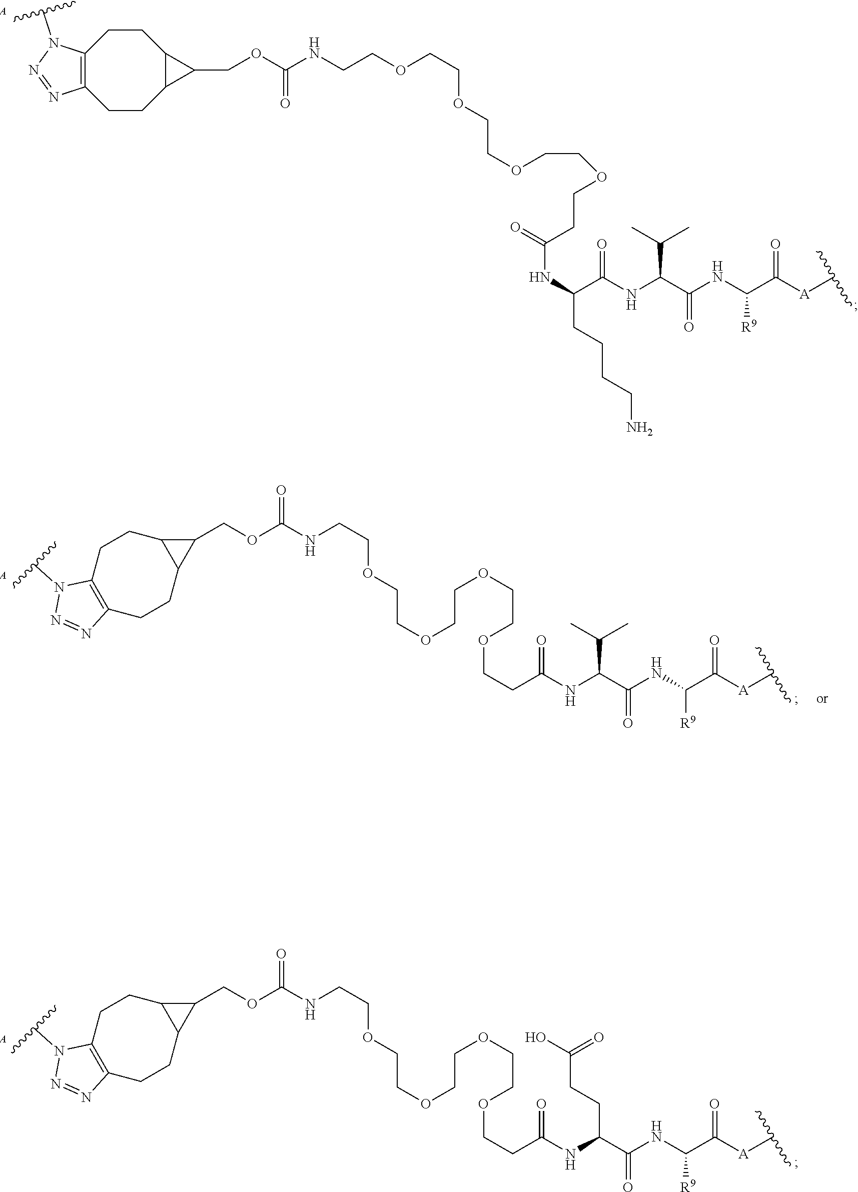

wherein
each

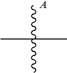

is a bond to a binding protein of this disclosure;
each

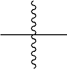

is a bond to the payload (e.g., wherein “payload” is a GR agonist of Structure (I) or (II));
each R⁹ is —CH₃ or —(CH₂)₃N(H)C(O)NH₂; and each A is —O—, —N(H)—

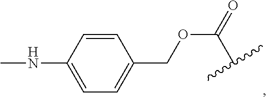 [SMILES: *C(=O)OCc1ccc(NC)cc1]

or where ZZ is hydrogen, alkylene, heteroalkylene, or a side

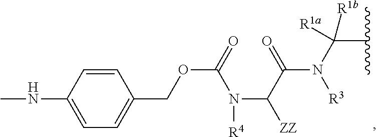

chain for an amino acid as discussed elsewhere herein. As discussed above, the bond to a binding protein of this disclosure can be direct, or via a spacer. In some embodiments, the bond to a binding protein of this disclosure is via a PEG spacer to a glutamine residue of a binding protein of this disclosure.

In any of the above embodiments, the (AA)ₙ group can be modified with one or more enhancement groups. Advantageously, the enhancement group can be linked to the side chain of any amino acid in (AA)ₙ. Useful amino acids for linking enhancement groups include lysine, asparagine, aspartate, glutamine, glutamate, and citrulline. The link to the enhancement group can be a direct bond to the amino acid side chain, or the link can be indirect via a spacer or reactive group. Useful spacers and reactive groups include any described above. The enhancement group can be any group deemed useful by those of skill in the art. For example, the enhancement group can be any group that imparts a beneficial effect to the compound, prodrug, payload (e.g., wherein “payload” is a GR agonist of Structure (I) or (II)), linker-payload, or conjugate including biological, biochemical, synthetic, solubilizing, imaging, detecting, and reactivity effects, and the like. In some embodiments, the enhancement group is a hydrophilic group. In some embodiments, the enhancement group is a cyclodextrin. In some embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In some embodiments, sugars include, without limitation, monosaccharides, disaccharides, and polysaccharides. Exemplary monosaccharides include glucose, ribose, deoxyribose, xylose, arabinose, mannose, galactose, fructose, and the like. In some embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In some embodiments, the cyclodextrin is alpha cyclodextrin, beta cyclodextrin, or gamma cyclodextrin, or mixtures thereof. In some embodiments, the cyclodextrin is alpha cyclodextrin. In some embodiments, the cyclodextrin is beta cyclodextrin. In some embodiments, the cyclodextrin is gamma cyclodextrin. In some embodiments, the enhancement group can improve solubility of the remainder of the conjugate. In some embodiments, the alkyl, heteroalkyl, alkylenyl, or heteroalkylenyl sulfonic acid is substituted or non-substituted. In some embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5. In some embodiments, the linker is:

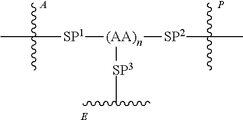

wherein:
SP¹ is a spacer; SP² is a spacer; SP³ is a spacer, linked to one AA of (AA)ₙ;

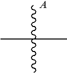

is one or more bonds to a binding protein of this disclosure;

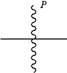

is one or more bonds to the payload (e.g., wherein “payload” is a GR agonist of Structure (I) or (II));

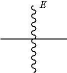

is one or more bonds to the enhancement group EG; each AA is an amino acid; and n is an integer from 1 to 10.

As discussed above, the bond to a binding protein of this disclosure can be direct, or via a spacer. In some embodiments, the bond to a binding protein of this disclosure f is via a PEG spacer to a glutamine residue of a binding protein of this disclosure.

The SP¹ spacer group is as described above. The SP² spacer group is as described above. Each (AA)ₙ group is as described above.

The SP³ spacer is a moiety that connects the (AA)ₙ moiety to the enhancement group (EG). Suitable SP³ spacers include those comprising alkylene or polyether, or both. The ends of the SP³ spacers, i.e., the portion of the SP³ spacer directly bonded to the enhancement group or an AA, can be moieties derived from reactive moieties that are used for purposes of coupling the enhancement group or an AA to the SP³ spacer during the chemical synthesis of the conjugate. In some examples, the ends of the SP³ spacers, i.e., the portion of the spacer directly bonded to the enhancement group or an AA, can be residues of reactive moieties that are used for purposes of coupling the enhancement group or an AA to the spacer during the chemical synthesis of the conjugate. In some embodiments, SP³ is a spacer, linked to one and only one AA of (AA)ₙ. In some embodiments, the SP³ spacer is linked to the side chain of a lysine residue of (AA)ₙ.

n some embodiments, the SY³ spacer is:

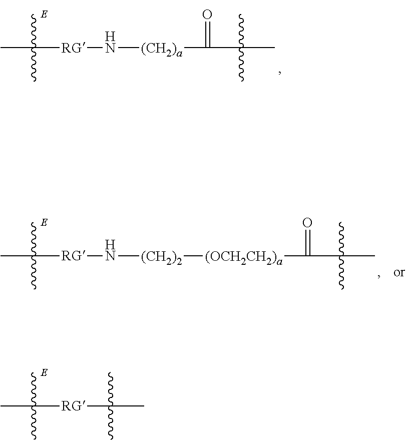

wherein:
RG′ is a reactive group residue following reaction of a reactive group RG with an enhancement agent EG;

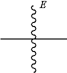

is a bond to the enhancement agent;

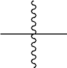

is a bond to (AA)ₙ;
a is an integer from 2 to 8; and
n is an integer from 1 to 4.

The reactive group RG can be any reactive group known to those of skill in the art to be capable of forming one or more bonds to the enhancement agent. The reactive group RG is a moiety comprising a portion in its structure that can react with the enhancement group to form a linker. Following conjugation to the enhancement group, the reactive group becomes the reactive group residue (RG′). The reactive group RG can be any reactive group described above. Illustrative reactive groups include those that comprise haloacetyl, isothiocyanate, succinimide, N-hydroxysuccinimide, or maleimide portions that can react with a binding protein of this disclosure.

In some embodiments, reactive groups include alkynes. In some embodiments, the alkynes are alkynes capable of undergoing 1,3-cycloaddition reactions with azides in the absence of copper catalysts such as strained alkynes. Strained alkynes are suitable for strain-promoted alkyne-azide cycloadditions (SPAAC), cycloalkynes, e.g., cyclooctynes, and benzannulated alkynes. Suitable alkynes include moieties having one of the following structures:

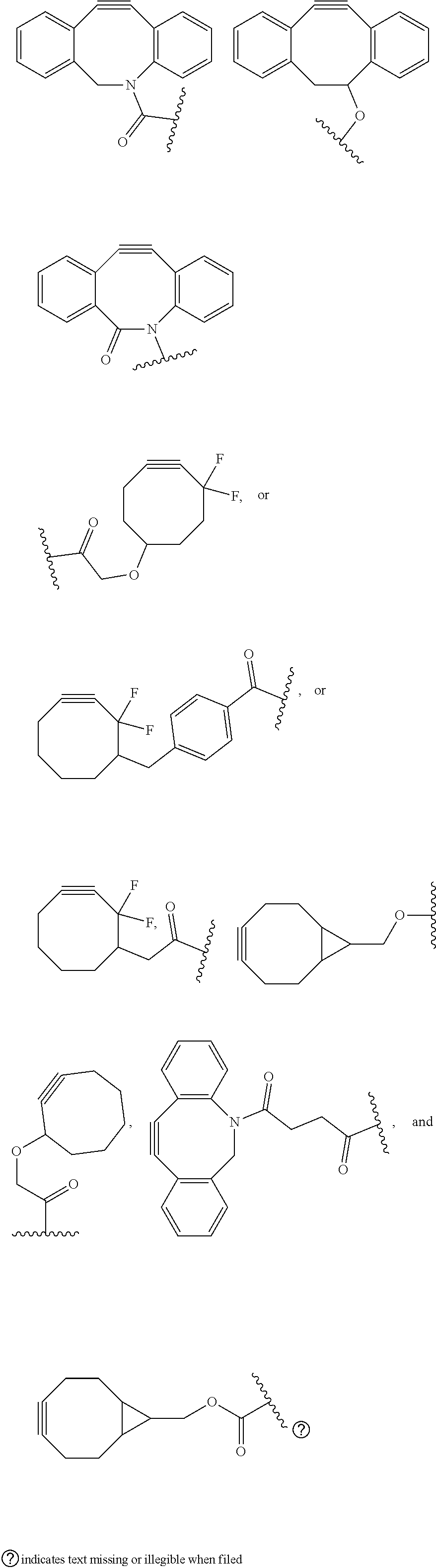

In some embodiments, the linker is:

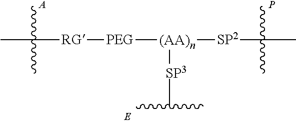

wherein:
RG′ is a reactive group residue following reaction of a reactive group RG with a binding protein of this disclosure;
PEG is NH PEG4-C(O)—;
SP² is a spacer;
SP³ is a spacer, linked to one AA residue of (AA)ₙ;

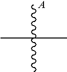

is one or more bonds to a binding protein of this disclosure;

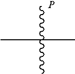

is one or more bonds to the payload (e.g., wherein “payload” is a GR agonist of Structure (I) or (II));

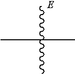

is one or more bonds to the enhancement group EG;
each AA is an amino acid residue; and
n is an integer from 1 to 10.

As discussed above, the bond to a binding protein of this disclosure can be direct, or via a spacer. In some embodiments, the bond to a binding protein of this disclosure is via a PEG spacer to a glutamine residue of the polypeptide.

In some embodiments, the linker is:

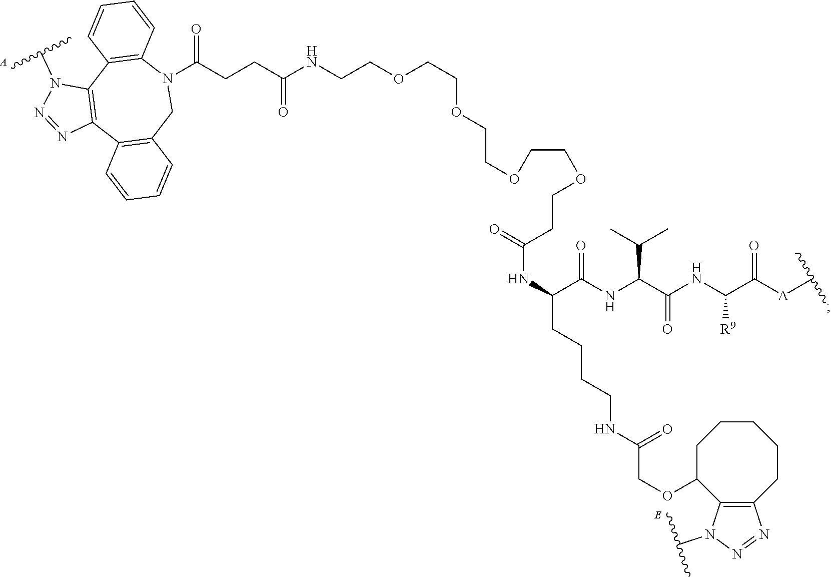

or a pharmaceutically acceptable salt, solvate, or stereoisomeric form thereof, or a regioisomer thereof, or a mixture of regioisomers thereof, wherein:
each

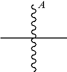

is a bond to a binding protein of this disclosure;
each

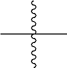

is a bond to the payload (e.g., wherein “payload” is a GR agonist of Structure (I) or (II));
each

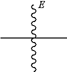

is a bond to the enhancement agent;
each R⁹ is —CH₃ or —(CH₂)₃N(H)C(O)NH₂; and
each A is —O—, —N(H)—,

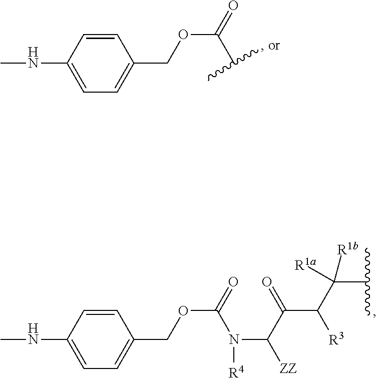

where ZZ is hydrogen, alkylene, heteroalkylene, or a side chain for an amino acid as discussed elsewhere herein. In some embodiments, 1,3-cycloaddition or SPAAC regioisomers, or mixture of regioisomers, are derived from PEG-N₃ derivatized antibodies treated with suitable alkynes. For example, in one embodiment, the linker is:

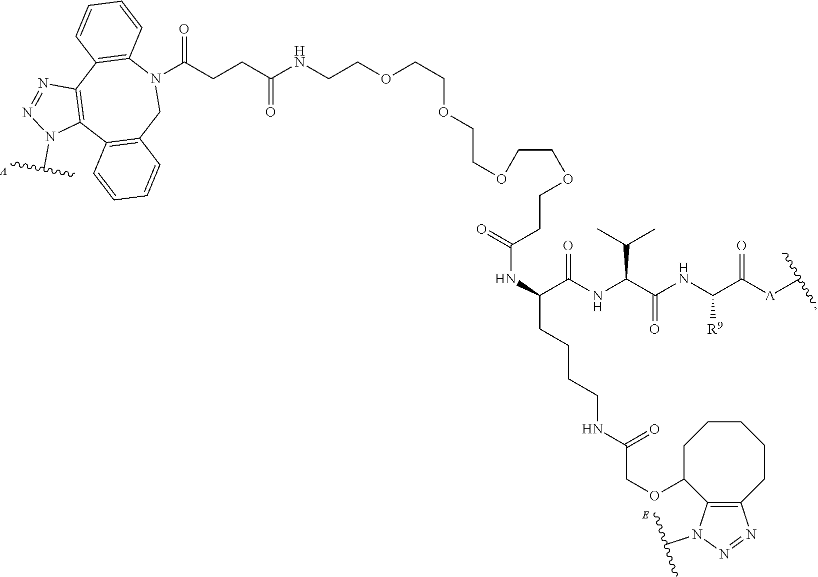

or a pharmaceutically acceptable salt, solvate, or stereoisomeric form thereof, or a regioisomer thereof, or a mixture of regioisomers thereof. By way of further example, in one embodiment, the linker is:

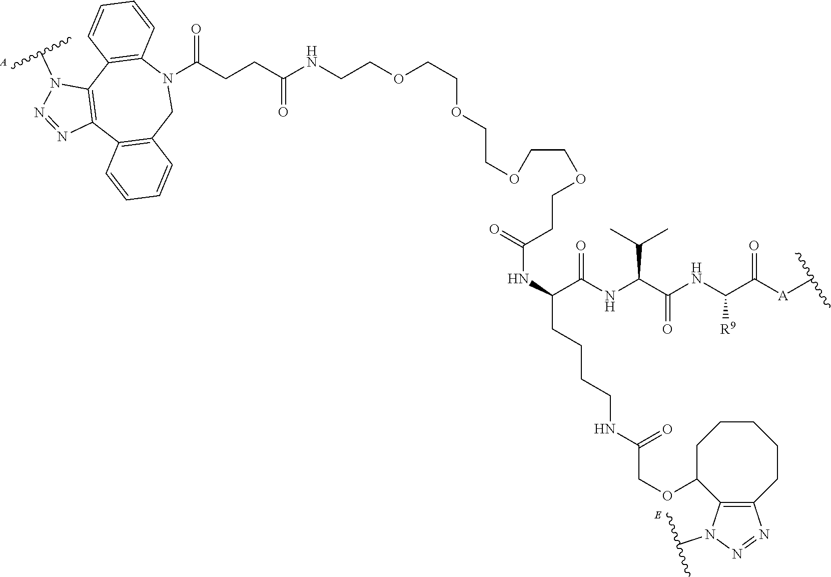

or a pharmaceutically acceptable salt, solvate, or stereoisomeric form thereof, or a regioisomer thereof, or a mixture of regioisomers thereof. By way of further example, the linker is:

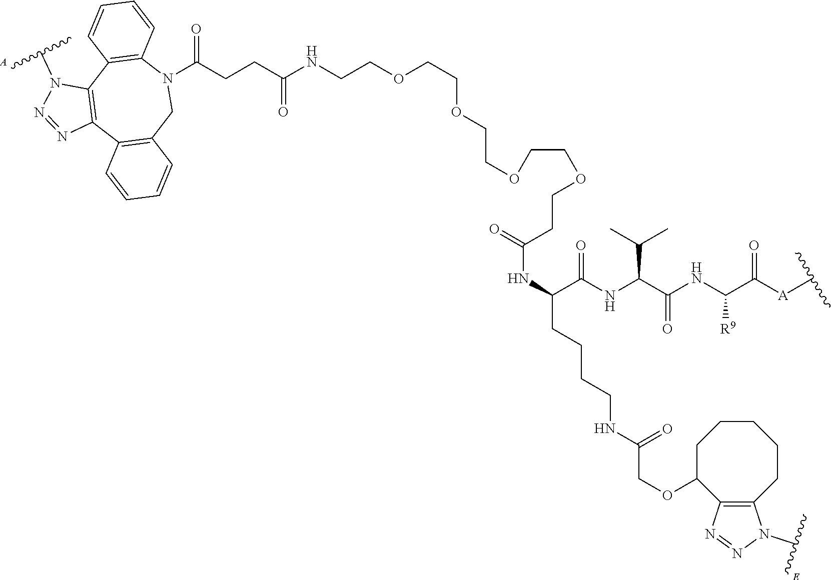

or a pharmaceutically acceptable salt, solvate, or stereoisomeric form thereof, or a regioisomer thereof, or a mixture of regioisomers thereof. By way of further example, in one embodiment, the linker is:

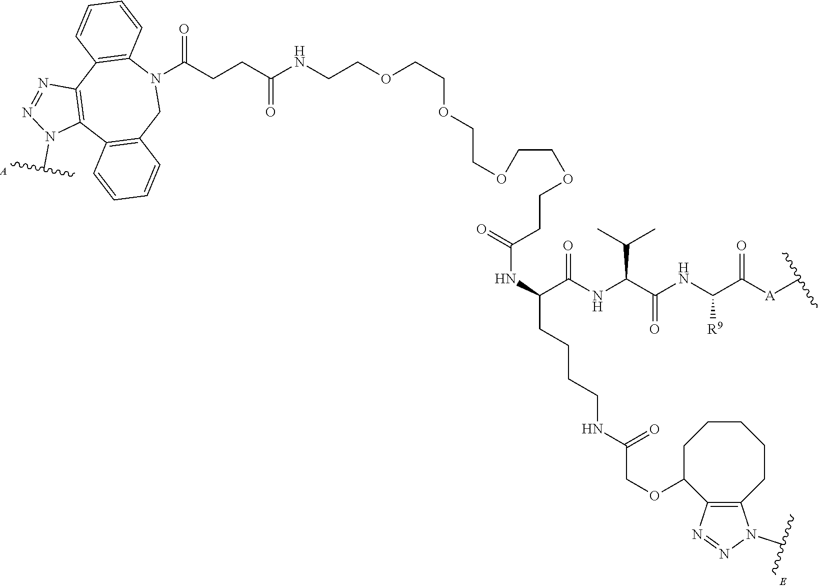

or a pharmaceutically acceptable salt, solvate, or stereoisomeric form thereof, or a regioisomer thereof, or a mixture of regioisomers thereof. As discussed above, the bond to a binding protein of this disclosure can be direct, or via a spacer. In some embodiments, the bond to a binding protein of this disclosure is via a PEG spacer to a glutamine residue of the polypeptide. In some embodiments, the enhancement agent is a hydrophilic group. In some embodiments, the enhancement agent is cyclodextrin. In some embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In some embodiments, sugars include, without limitation, monosaccharides, disaccharides, and polysaccharides. Exemplary monosaccharides include glucose, ribose, deoxyribose, xylose, arabinose, mannose, galactose, fructose, and the like. In some embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In some embodiments, the cyclodextrin is alpha cyclodextrin, beta cyclodextrin, or gamma cyclodextrin, or mixtures thereof. In some embodiments, the cyclodextrin is alpha cyclodextrin. In some embodiments, the cyclodextrin is beta cyclodextrin. In some embodiments, the cyclodextrin is gamma cyclodextrin. In some embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH)₁₋₅SO₃H, —(CH₂CH₂O)ₘ C(O)NH—(CH₂)₁₋₅SO₃H, —(CH₂)ₙ—N((CH₂)₁₋₅C(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is

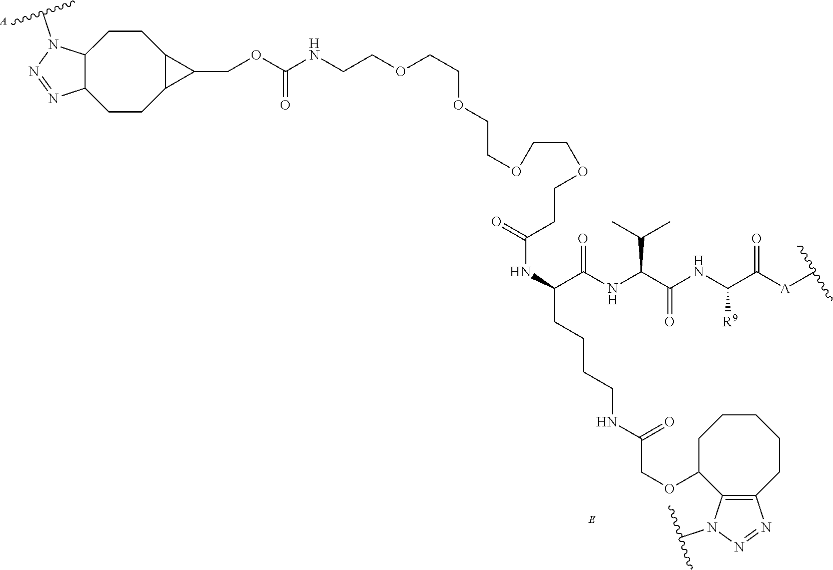

or a pharmaceutically acceptable salt, solvate, or stereoisomeric form thereof, or a regioisomer thereof, or mixture of regioisomers thereof, wherein:
each

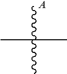

is a bond to a binding protein of this disclosure;
each

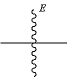

is a bond to the enhancement agent;
each

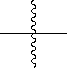

is a bond to the payload (e.g., wherein “payload” is a GR agonist of Structure (I) or (II));
each R⁹ is —CH₃ or —(CH₂)₃N(H)C(O)NH₂; and
each A is O, —N(H)—,

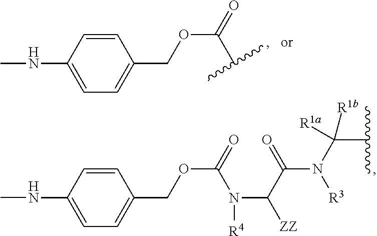

where ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to a binding protein of this disclosure thereof is via a PEG spacer to a glutamine residue of the polypeptide. In some embodiments, the enhancement agent is a hydrophilic group. In some embodiments, the enhancement agent is cyclodextrin. In some embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In some embodiments, sugars include, without limitation, monosaccharides, disaccharides, and polysaccharides. Exemplary monosaccharides include glucose, ribose, deoxyribose, xylose, arabinose, mannose, galactose, fructose, and the like. In some embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In some embodiments, the cyclodextrin is alpha cyclodextrin, beta cyclodextrin, or gamma cyclodextrin, or mixtures thereof. In some embodiments, the cyclodextrin is alpha cyclodextrin. In some embodiments, the cyclodextrin is beta cyclodextrin. In some embodiments, the cyclodextrin is gamma cyclodextrin. In some embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂) i-SC(O)NH(CH₂)₁₋₅SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is (CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is

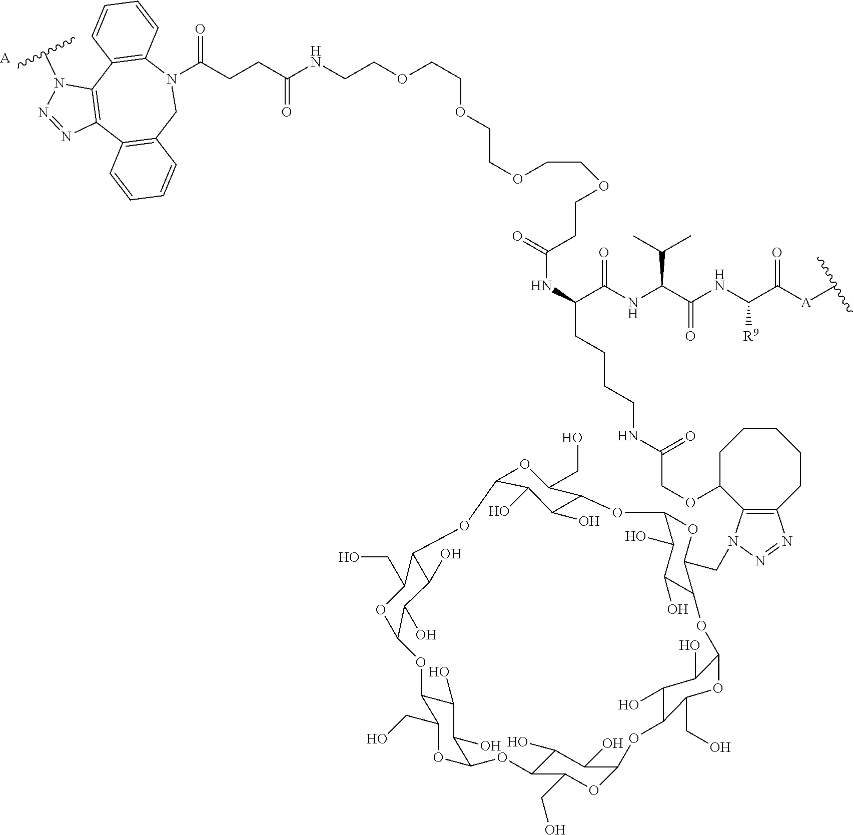

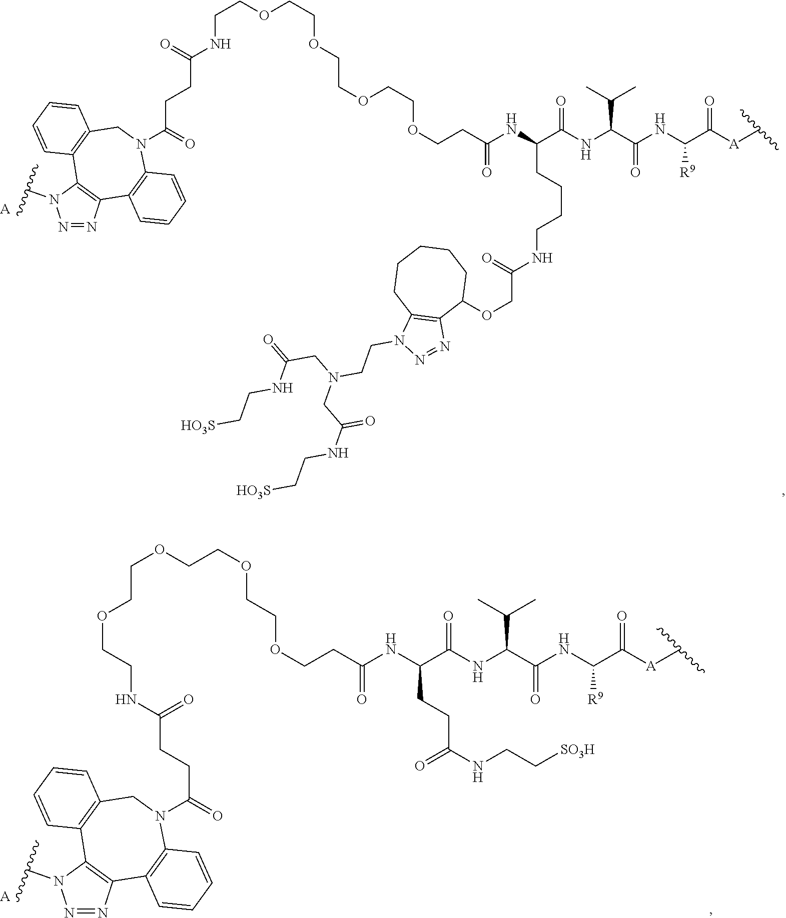

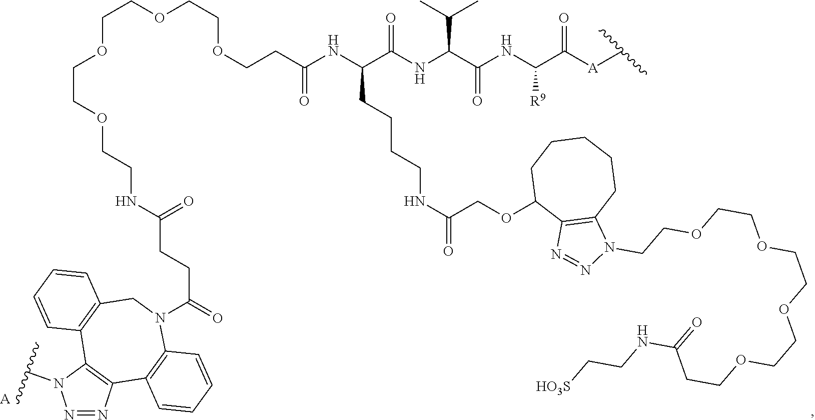

or a pharmaceutically acceptable salt, solvate, or stereoisomeric form thereof, or a regioisomer thereof, or mixture of regioisomers thereof, wherein:
each

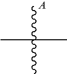

is a bond to a binding protein of this disclosure;
each

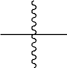

is a bond to the payload (e.g., wherein “payload” is a GR agonist of Structure (I) or (II));
R⁹ is —CH₃ or —(CH₂)₃N(H)C(O)NH₂; and
A is —O—, —N(H)—,

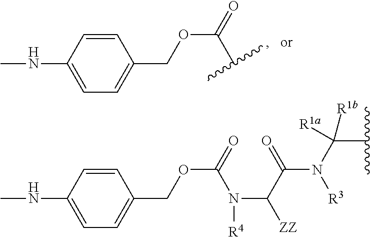

where ZZ is hydrogen, alkylene, heteroalkylene, or a side chain for an amino acid as discussed elsewhere herein. As discussed above, the bond to the polypeptide can be direct, or via a spacer.

In some embodiments, the bond to a binding protein of this disclosure is via a PEG spacer to a glutamine residue of the polypeptide.

In some embodiments, the linker is:

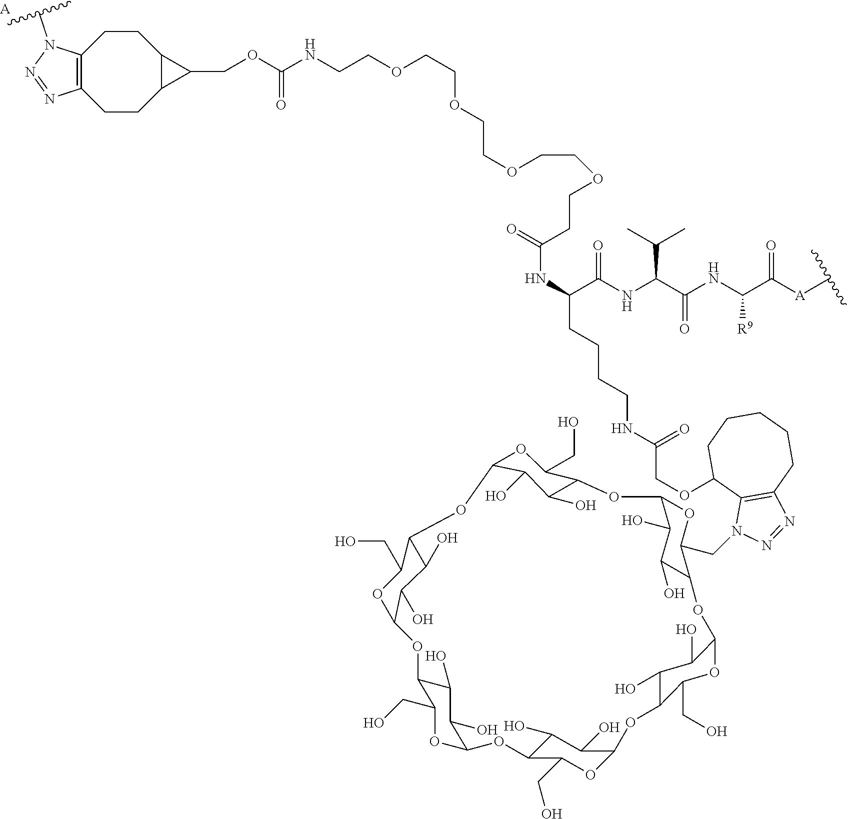

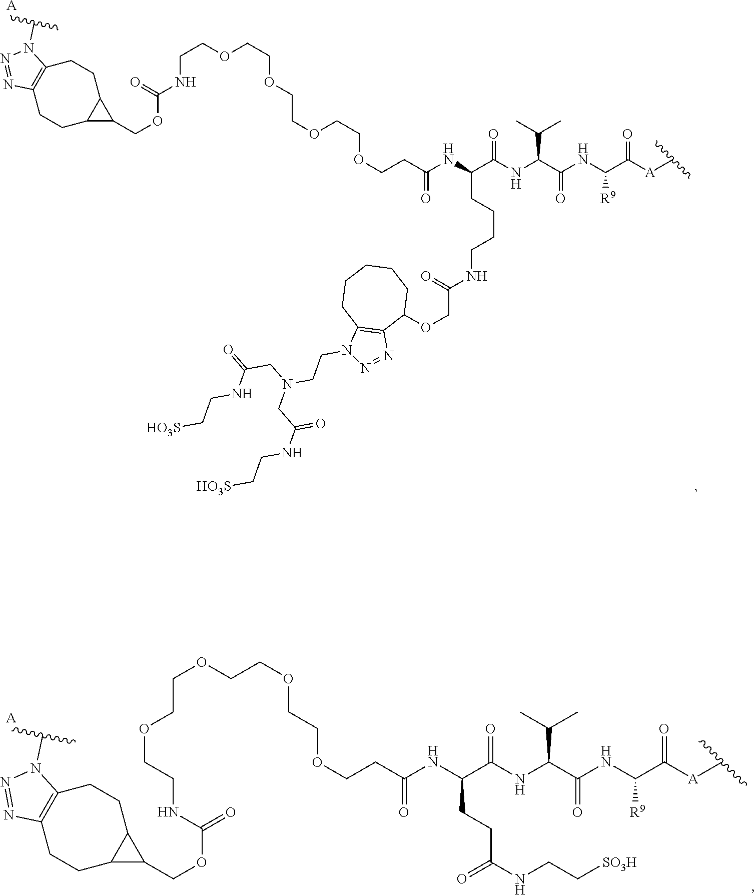

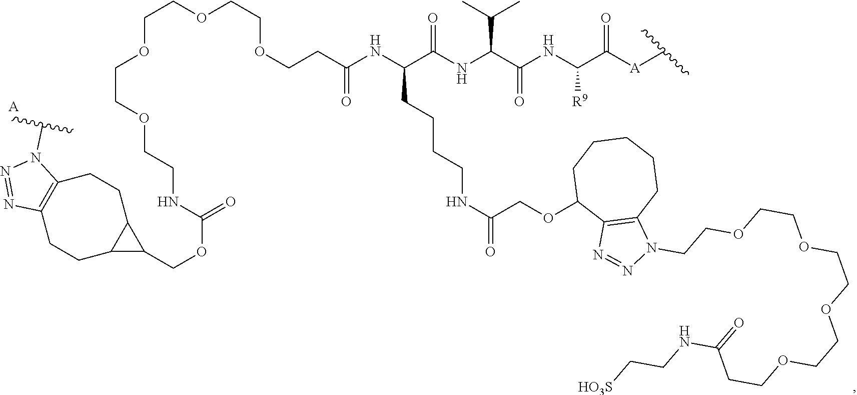

or a pharmaceutically acceptable salt, solvate, or stereoisomeric form thereof, or a regioisomer thereof, or mixture of regioisomers thereof, wherein:
each

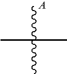

is a bond to a binding protein of this disclosure;
each

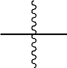

is a bond to the payload (e.g., wherein “payload” is a GR agonist of Structure (I) or (II));
R⁹ is —CH₃ or —(CH₂)₃N(H)C(O)NH₂; and
A is —O—, —N(H)—,

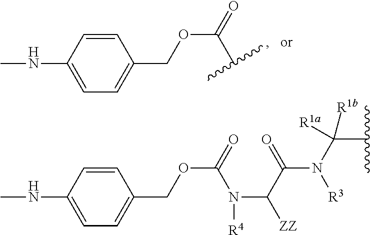

where ZZ is hydrogen, alkylene, heteroalkylene, or a side chain for an amino acid as discussed elsewhere herein. As discussed above, the bond to the peptide can be direct, or via a spacer.

In some embodiments, the bond to a binding protein of this disclosure is via a PEG spacer to a glutamine residue of the polypeptide.

In some embodiments, the linker is:

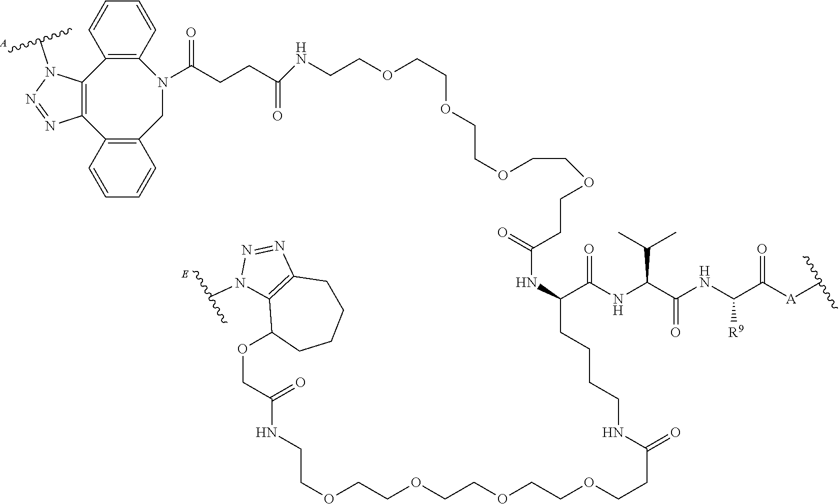

or a pharmaceutically acceptable salt, solvate, or stereoisomeric form thereof, or a regioisomer thereof, or mixture of regioisomers thereof, wherein:

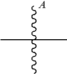

each is a bond to a binding protein of this disclosure;

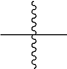

each is a bond to the payload (e.g., wherein “payload” is a GR agonist of Structure (I) or (II);

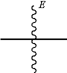

each is a bond to the enhancement group;
each R⁹ is —CH₃ or —(CH₂)₃N(H)C(O)NH₂; and
each A is —O—, —N(H)—,

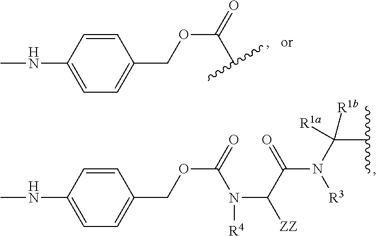

where ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to a binding protein of this disclosure is via a PEG spacer to a glutamine residue of the polypeptide. In some embodiments, the enhancement agent is a hydrophilic group. In some embodiments, the enhancement agent is cyclodextrin. In some embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In some embodiments, sugars include, without limitation, monosaccharides, disaccharides, and polysaccharides.

Exemplary monosaccharides include glucose, ribose, deoxyribose, xylose, arabinose, mannose, galactose, fructose, and the like. In some embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In some embodiments, the cyclodextrin is alpha cyclodextrin, beta cyclodextrin, or gamma cyclodextrin, or mixtures thereof. In some embodiments, the cyclodextrin is alpha cyclodextrin. In some embodiments, the cyclodextrin is beta cyclodextrin. In some embodiments, the cyclodextrin is gamma cyclodextrin. In some embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂) i-SC(O)NH(CH₂) i-s SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is (CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is:

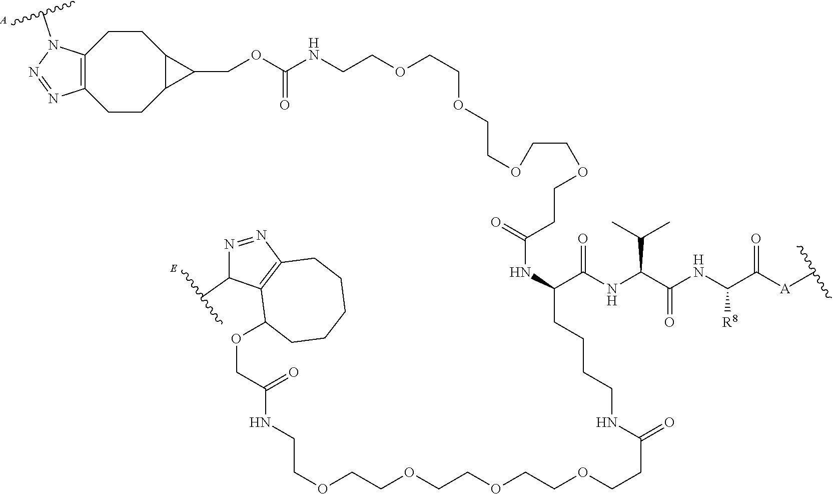

or a pharmaceutically acceptable salt, solvate, or stereoisomeric form thereof, or a regioisomer thereof, or mixture of regioisomers thereof, wherein:
each

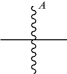

is a bond to a binding protein of this disclosure;
each

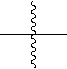

is a bond to the payload (e.g., wherein “payload” is a GR agonist of Structure (I) or (II));
each R⁹ is —CH₃ or —(CH₂)₃N(H)C(O)NH₂; and
each A is —O—, —N(H)—,

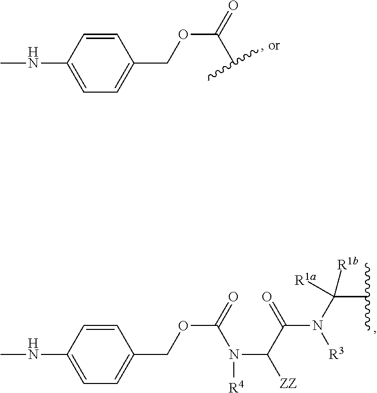

where ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to a binding protein of this disclosure is via a PEG spacer to a glutamine residue of the polypeptide. In some embodiments, the enhancement agent is a hydrophilic group. In some embodiments, the enhancement agent is cyclodextrin. In some embodiments, the enhancement group is an alkyl, heteroalkyl, alkylenyl, heteroalkylenyl sulfonic acid, heteroalkylenyl taurine, heteroalkylenyl phosphoric acid or phosphate, heteroalkylenyl amine (e.g., quaternary amine), or heteroalkylenyl sugar. In some embodiments, sugars include, without limitation, monosaccharides, disaccharides, and polysaccharides. Exemplary monosaccharides include glucose, ribose, deoxyribose, xylose, arabinose, mannose, galactose, fructose, and the like. In some embodiments, sugars include sugar acids such as glucuronic acid, further including conjugated forms such as glucuronides (i.e., via glucuronidation). Exemplary disaccharides include maltose, sucrose, lactose, lactulose, trehalose, and the like. Exemplary polysaccharides include amylose, amylopectin, glycogen, inulin, cellulose, and the like. The cyclodextrin can be any cyclodextrin known to those of skill. In some embodiments, the cyclodextrin is alpha cyclodextrin, beta cyclodextrin, or gamma cyclodextrin, or mixtures thereof. In some embodiments, the cyclodextrin is alpha cyclodextrin. In some embodiments, the cyclodextrin is beta cyclodextrin. In some embodiments, the cyclodextrin is gamma cyclodextrin. In some embodiments, the alkyl, heteroalkyl, alkylenyl, or heteroalkylenyl sulfonic acid is —(CH₂)₁₋₅SO₃H, —(CH₂)ₙ—NH—(CH₂)₁₋₅SO₃H, —(CH₂)ₙ—C(O)NH—(CH₂)₁₋₅SO₃H, —(CH₂CH₂O)ₘ—C(O)NH—(CH₂)₁₋₅SO₃H, —(CH₂)ₙ—N((CH₂) i-SC(O)NH(CH₂) i-s SO₃H)₂, —(CH₂)ₙ—C(O)N((CH₂)₁₋₅C(O)NH(CH₂)₁₋₅SO₃H)₂, or —(CH₂CH₂O)ₘ—C(O)N((CH₂)₁₋₅C(O)NH(CH₂)₁₋₅SO₃H)₂, wherein n is 1, 2, 3, 4, or 5, and m is 1, 2, 3, 4, or 5. In one embodiment, the alkyl or alkylenyl sulfonic acid is —(CH₂)₁₋₅SO₃H. In another embodiment, the heteroalkyl or heteroalkylenyl sulfonic acid is —(CH₂)ₙ—NH—(CH₂)₁₋₅SO₃H, wherein n is 1, 2, 3, 4, or 5. In another embodiment, the alkyl, heteroalkyl, alkylenyl, or heteroalkylenyl sulfonic acid is —(CH₂)ₙ—C(O)NH—(CH₂)₁₋₅SO₃H, wherein n is 1, 2, 3, 4, or 5. In another embodiment, the alkyl, heteroalkyl, alkylenyl, or heteroalkylenyl sulfonic acid is (CH₂CH₂O)ₘ—C(O)NH—(CH₂)₁₋₅SO₃H, wherein m is 1, 2, 3, 4, or 5. In another embodiment, the alkyl, heteroalkyl, alkylenyl, or heteroalkylenyl sulfonic acid is —(CH₂)ₙ—N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂)ₙ—C(O)N((CH₂)₁₋₅C(O)NH(CH₂)₁₋₅SO₃H)₂, wherein n is 1, 2, 3, 4, or 5. In another embodiment, the alkyl, heteroalkyl, alkylenyl, or heteroalkylenyl sulfonic acid is —(CH₂CH₂O)ₘ—C(O)N((CH₂)₁₋₅C(O)NH(CH₂)₁₋₅SO₃H)₂, wherein m is 1, 2, 3, 4, or 5.

In some embodiments, the linker is

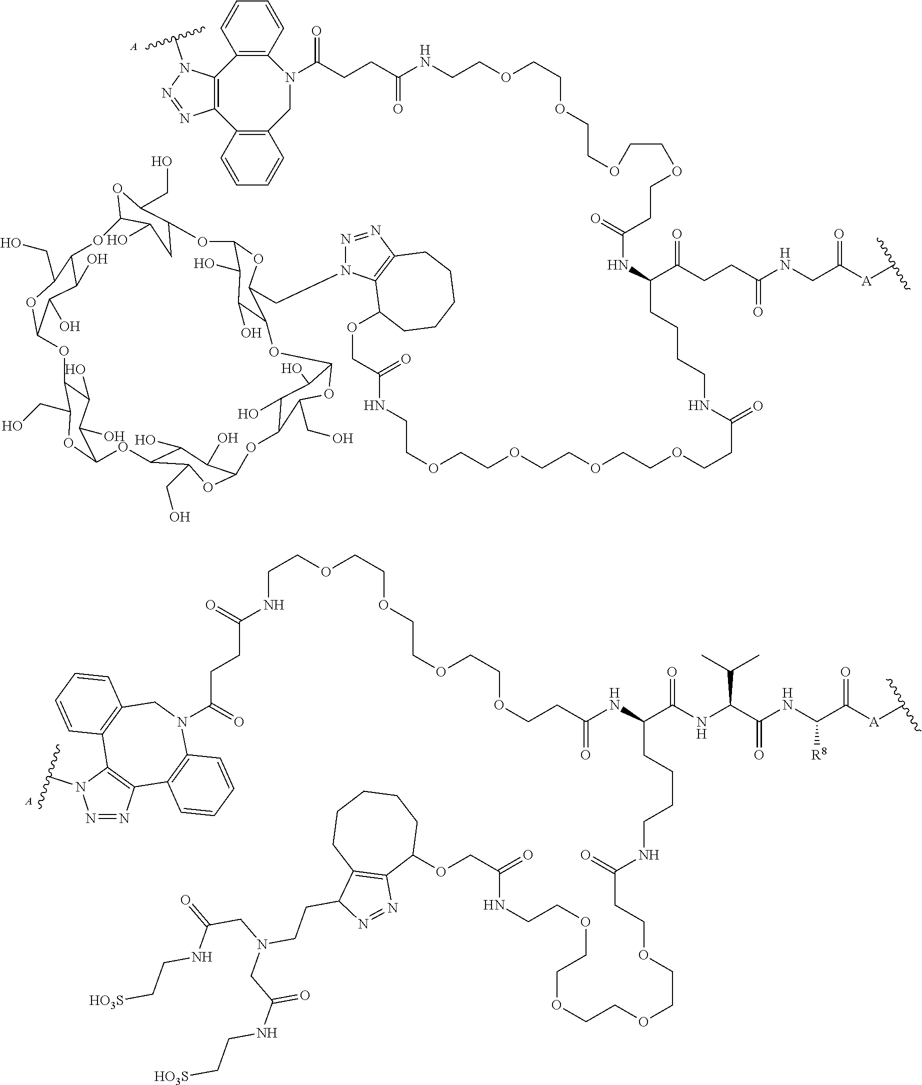

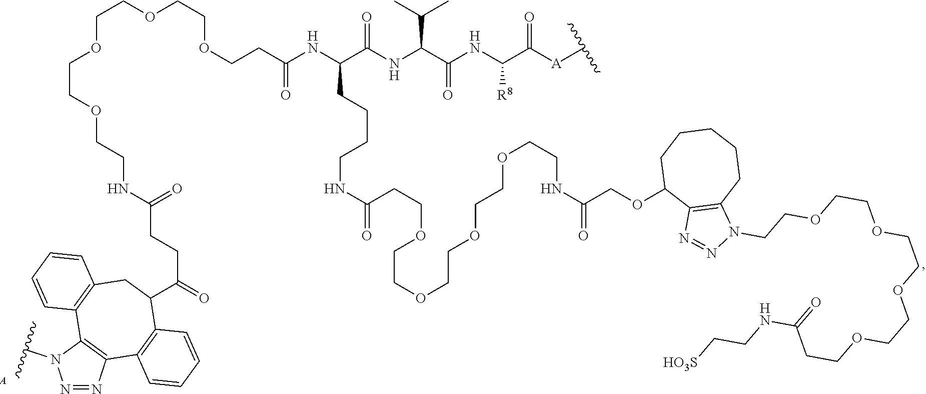

or a pharmaceutically acceptable salt, solvate, or stereoisomeric form thereof, or a regioisomer thereof, or mixture of regioisomers thereof, wherein:
each

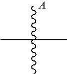

is a bond to a binding protein of this disclosure;
each

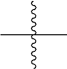

is a bond to the payload (e.g., wherein “payload” is a GR agonist of Structure (I) or (II));
R⁹ is —CH₃ or —(CH₂)₃(H)C(O)NH₂, and
A is —O—, —N(H)—,

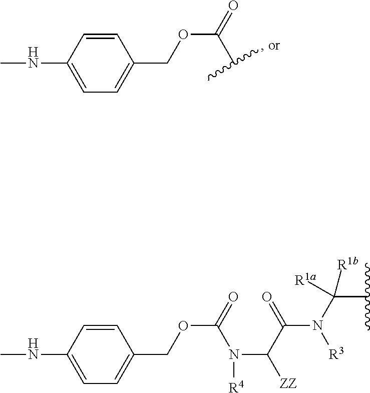

where ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to the polypeptide is via a PEG spacer to a glutamine residue of the polypeptide.

In some embodiments, the linker is:

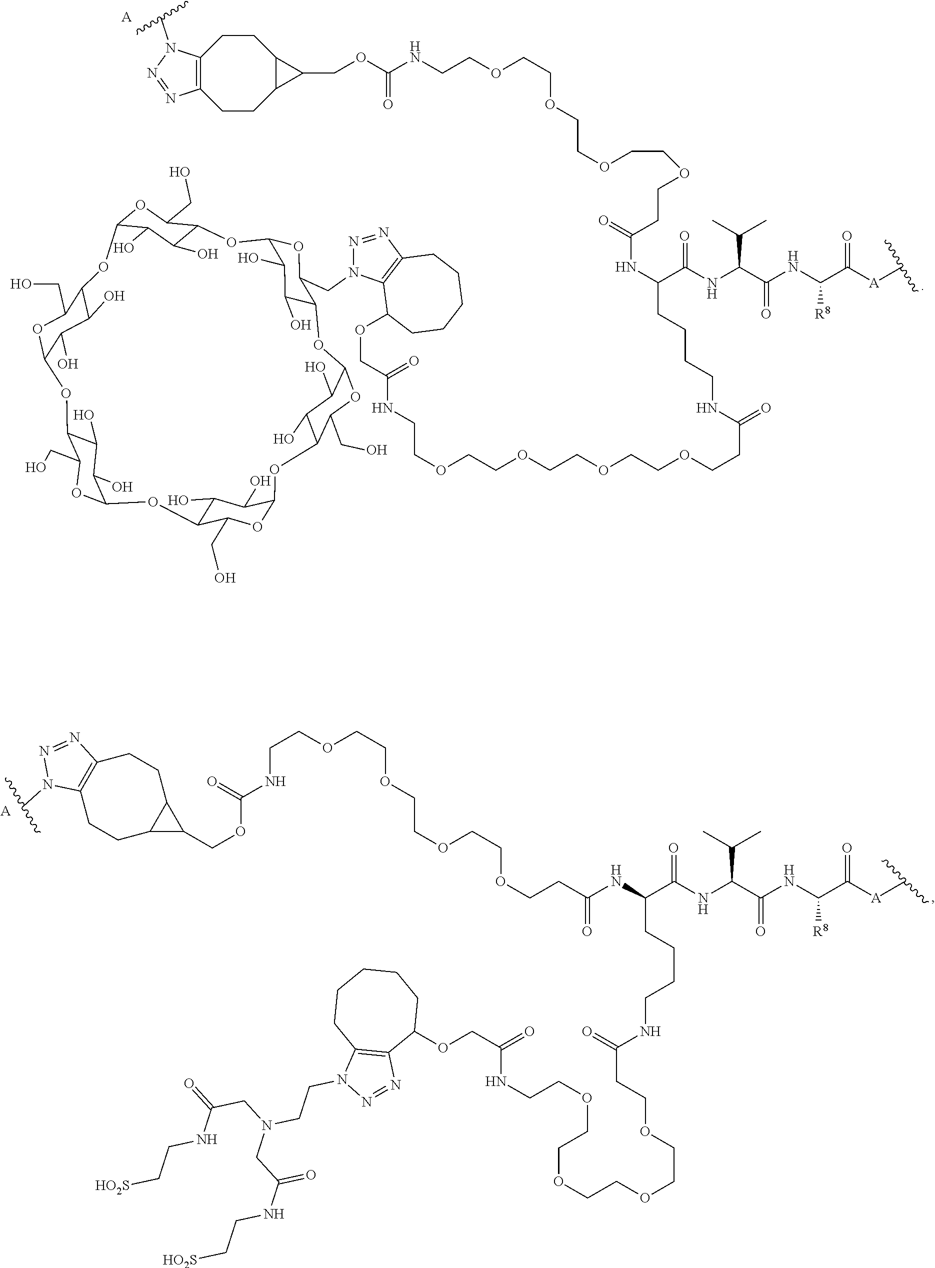

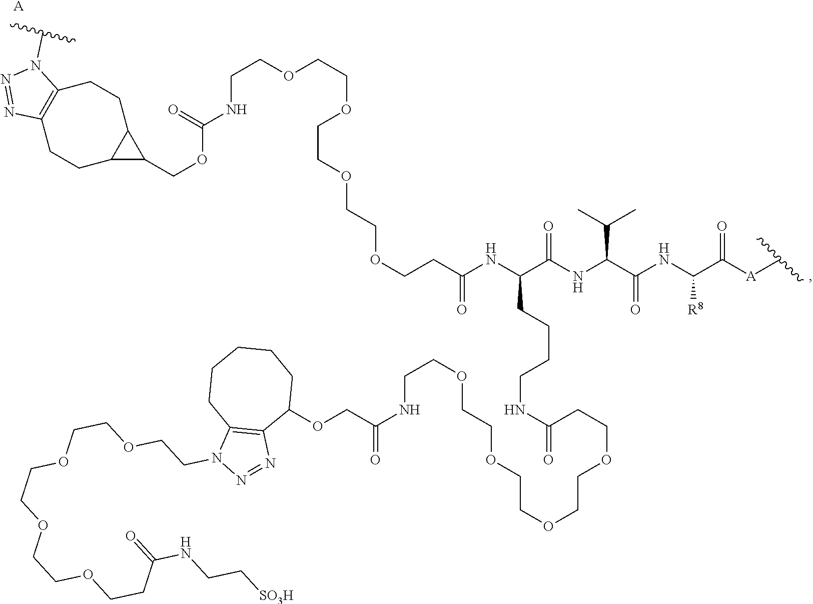

or a pharmaceutically acceptable salt, solvate, or stereoisomeric form thereof, or a regioisomer thereof, or mixture of regioisomers thereof, wherein:
each

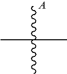

is a bond to a binding protein of this disclosure;
each

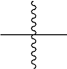

is a bond t the compound of Structure (I) or (II);
R⁹ is —CH₃ or —(CH₂)₃N(H)C(O)NH₂; and
A is —O—, —N(H)—, where

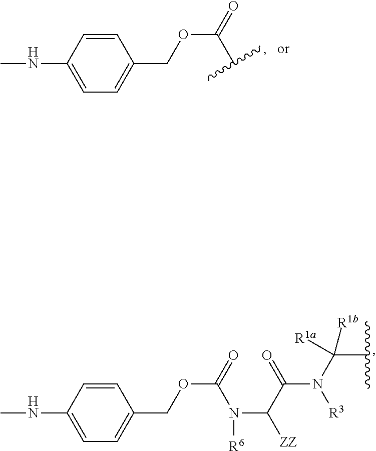

ZZ is hydrogen, alkylene, heteroalkylene, or a side chain for an amino acid as discussed elsewhere herein. As discussed above, the bond to a binding protein of this disclosure can be direct, or via a spacer. In some embodiments, the bond to the polypeptide is via a PEG spacer to a glutamine residue of the polypeptide.

11. Category XI Linkers

In certain embodiments, the present disclosure provides conjugates comprising branched phenyl maleimide linkers (e.g., linkers of Structure (Ix)) that enable the formation of a covalent bond between a linker-payload and a protein. As noted above, in certain embodiments of the present disclosure, compounds useful as linkers between payloads (including fragments thereof) and targeting peptides (including fragments thereof—e.g., antibodies) are provided.

Accordingly, some embodiments provide a linker having the following Structure (Ix):

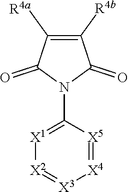

wherein:
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a compound of Structure (I) or (II), and combinations thereof;
   provided that at least one of R¹ and R² comprises a compound of Structure (I) or (II);
each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
R^{4a} and R^{4b} are each independently hydrogen, deuterium, halo, or —S—R^{4c} wherein R^{4c} is substituted or unsubstituted C₆-C₁₀ aryl or substituted or unsubstituted 5-12 membered heteroaryl; and
L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene, or combinations thereof;
   as a stereoisomer, enantiomer or tautomer thereof or a mixture thereof, or a pharmaceutically acceptable salt, solvate or prodrug thereof.

In some embodiments, R^{4a} and R^{4b} are both hydrogen. In some embodiments, R^{4a} is halo or R^{4b} is halo. In some embodiments, R^{4a}, R^{4b}, or both have one of the following structures:

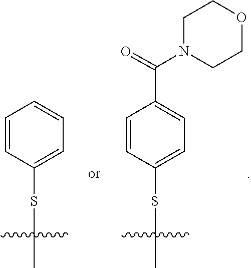 [SMILES: *Sc1ccccc1 ; *Sc1ccc(C(=O)N2CCOCC2)cc1]

In some embodiments, R¹, R², and/or R^{3a} comprises elements selected from an amino acid element, a charged element, a heteroalkylene element, a hydrophilic element, a trigger element, an immolative element, a polar cap, a compound of Structure (I) or (II), and combinations thereof. It is understood that these elements can be connected in any order and be connected in a linear manner or via a branched connection. In some embodiments, R¹ R², and/or R^{3a} comprises multiple occurrences of an element (e.g., two or more heteroalkylene elements, two or more hydrophilic elements, two or more polar caps, etc.).

In some embodiments, R¹, R², or R^{3a} comprises a branch point as part of an amino acid element (e.g., lysine) wherein additional elements are attached via an epsilon amine of the lysine and other additional elements are linked to the amino acid element via one or more peptide bonds to the alpha carbon of a lysine. A similar motif could be utilized with a glutamic acid of an amino acid element. In some embodiments, an amino acid element comprises one of the following structures:

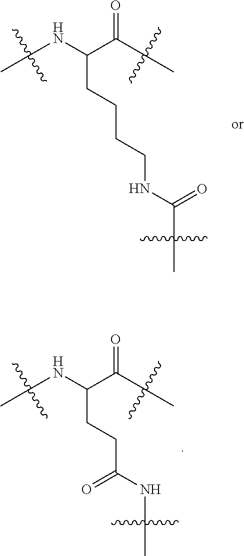 [SMILES: *NC(CCCCNC(*)=O)C(*)=O ; *NC(=O)CCC(N*)C(*)=O]

In some embodiments, a linker of Structure (Ix) comprises one of the following structures:

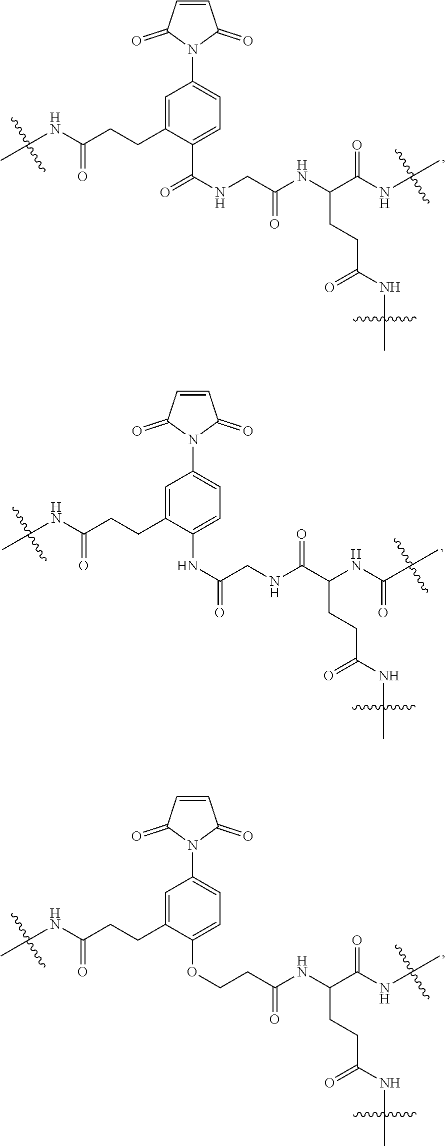 [SMILES: *NC(=O)CCc1cc(N2C(=O)C=CC2=O)ccc1C(=O)NCC(=O)NC(CCC(=O)N*)C(=O)N* ; *NC(=O)CCc1cc(N2C(=O)C=CC2=O)ccc1NC(=O)CNC(=O)C(CCC(=O)N*)NC(*)=O ; *NC(=O)CCc1cc(N2C(=O)C=CC2=O)ccc1OCCC(=O)NC(CCC(=O)N*)C(=O)N*]

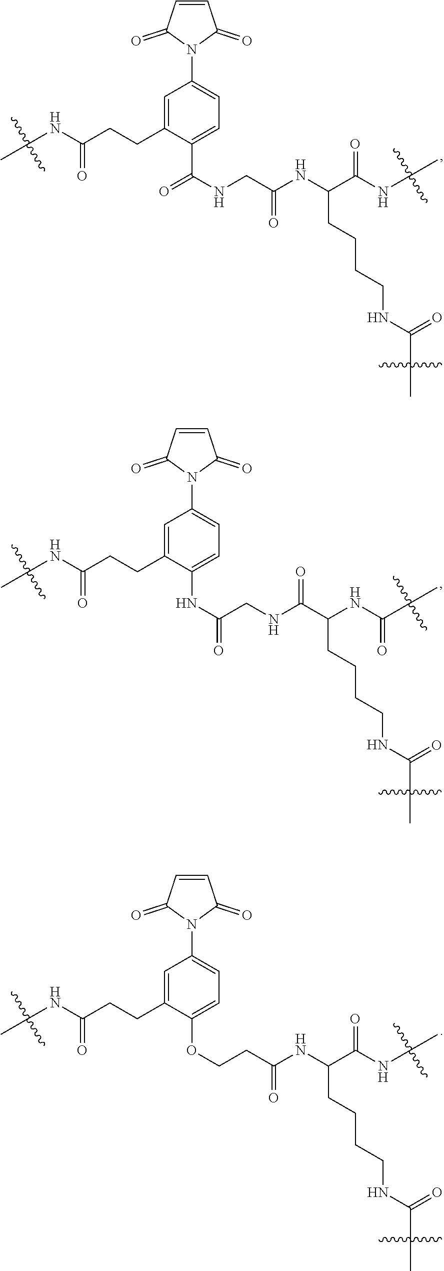 [SMILES: *NC(=O)CCc1cc(N2C(=O)C=CC2=O)ccc1C(=O)NCC(=O)NC(CCCCNC(*)=O)C(=O)N* ; *NC(=O)CCc1cc(N2C(=O)C=CC2=O)ccc1NC(=O)CNC(=O)C(CCCCNC(*)=O)NC(*)=O ; *NC(=O)CCc1cc(N2C(=O)C=CC2=O)ccc1OCCC(=O)NC(CCCCNC(*)=O)C(=O)N*]

In certain embodiments, R¹ has the following structure:

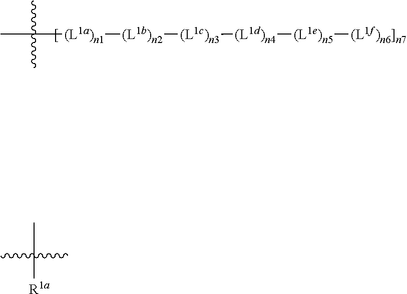

wherein:
L^{1a} is an amino acid element;
L^{1b} is a charged element;
L^{1v} is a heteroalkylene element;
L^{1d} is a hydrophilic element;
L^{1e} is a trigger element; and
L^{1f} is an immolative element;
wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a compound of Structure (I) or (II) that is covalently bound to one occurrence of L^{1a}, L^{1b}, L^{1c}, L^{1d}, L^{1e}, or L^{1f} and the compound of Structure (I) or (II) is optionally substituted with a polar cap.

In some embodiments, n7 is 1, 2, or 3. In some embodiments, n7 is 1 or 2. In some embodiments, n7 is 1.

In some embodiments, R¹ has the following structure:

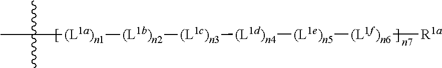

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a compound of Structure (I) or (II) optionally substituted with a polar cap.

In more embodiments, R² has the following structure:

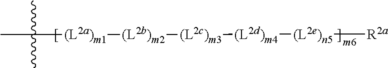

wherein:
L^{2a} is an amino acid element;
L^{2b} is a charged element;
L^{2c} is a heteroalkylene element;
L^{2d} is a hydrophilic element;
L^{2e} is a trigger element;
each occurrence of m1, m2, m3, m4, and m5 is independently an integer from 0-3, provided that m1+m2+m3+m4+m5=1 or more;
m6 is 1, 2, 3, 4, or 5; and
R^{2a} is hydrogen, alkyl, a compound of Structure (I) or (II), or a polar cap.

In some embodiments, m6 is 1, 2, or 3. In some embodiments, m6 is 1 or 2. In some embodiments, m6 is 1.

In still more embodiments, R^{3a} has the following structure:

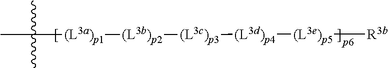

wherein:
L^{3a} is an amino acid element;
L^{3b} is a charged element;
L^{3c} is a heteroalkylene element;
L^{3d} is a hydrophilic element;
L^{3e} is a trigger element;
each occurrence of p1, p2, p3, p4, and p5 is independently an integer from 0-3, provided that p1+p2+p3+p4+p5=1 or more;
p6 is 1, 2, 3, 4, or 5; and
R^{3b} is hydrogen, alkyl, or a polar cap.

In some embodiments, p6 is 1, 2, or 3. In some embodiments, p6 is 1 or 2. In some embodiments, p6 is 1.

In certain embodiments, n7 is 1 and each of n1 through n6 are 1. In some embodiments, n7 is 1 and each of n1 through n4 are 0, n5 is 1, and n6 is 1. In certain embodiments, n7 is 1 and n1 is 0, n2 is 0, n3 is 1, n4 is 0, n5 is 1, and n6 is 1. In certain embodiments, n7 is 1 and n1 is 1, n2 is 0, n3 is 1, n4 is 0, n5 is 1, and n6 is 1. In certain embodiments, n7 is 1 and n1 is 1, n2 is 1, n3 is 1, n4 is 0, n5 is 1, and n6 is 1. In certain embodiments, n7 is 1 and n1 is 1, n2 is 1, n3 is 1, n4 is 1, n5 is 1, and n6 is 1. In certain embodiments, n7 is 1 and n1 is 1, n2 is 0, n3 is 1, n4 is 0, n5 is 1, and n6 is 1. In certain embodiments, n7 is 2. In certain embodiments, n7 is 3.

In some embodiments, m6 is 1 and each of m1 through m5 are 1. In some embodiments, m6 is 1, m1 is 1, m2 is 0, m3 is 0, m4 is 1, and m5 is 0. In some embodiments, m6 is 1, m1 is 1, m2 is 1, m3 is 0, m4 is 1, and m5 is 0. In some embodiments, m6 is 1, m1 is 1, m2 is 0, m3 is 1, m4 is 1, and m5 is 0. In some embodiments, m6 is 1, m1 is 1, m2 is 0, m3 is 0, m4 is 1, and m5 is 1. In some embodiments, m6 is 2. In certain embodiments, m6 is 3.

In some embodiments, p6 is 1 and each of p1 through p5 is 1. In certain embodiments, p6 is 1, p1 is 1, p2 is 0, p3 is 0, p4 is 1, and p5 is 0. In some embodiments, p6 is 1, p1 is 1, p2 is 1, p3 is 0, p4 is 1, and p5 is 0. In some embodiments, p6 is 1, p1 is 1, p2 is 0, p3 is 1, p4 is 1, and p5 is 0. In some embodiments, p6 is 1, p1 is 1, p2 is 0, p3 is 0, p4 is 1, and p5 is 1. In some embodiments, p6 is 2 and at least one occurrence of p1 is 1, p2 is 1, p3 is 0, p4 is 1, and p5 is 0. In some embodiments, p6 is 2. In certain embodiments, p6 is 3.

In some embodiments, an amino acid element comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine.

In certain embodiments, an amino acid element is selected from the group consisting of glycine, sarcosine, beta-alanine, and glutamic acid.

In some embodiments, an amino acid element comprises a dipeptide, a tripeptide, a tetrapeptide, or a pentapeptide.

In more embodiments, an amino acid element has one of the following structures:

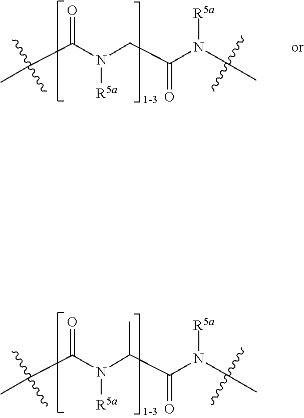

wherein each occurrence of R^{5a} is independently hydrogen, alkyl, hydroxyalkyl, or alkoxyalkyl.

In some embodiments, a charged element comprises moieties with a negative charge at pH 7.4 (i.e., a range from 6.3 to 8.5). In certain embodiments, a charged element comprises moieties with a positive charge at pH 7.4 (i.e., a range from 6.3 to 8.5).

In some embodiments, a charged element comprises one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof.

In certain embodiments, a charged amino acid is aspartic acid, glutamic acid, histidine, lysine, or arginine.

In some embodiments, R¹, R², or R^{3a} comprises a non-cleavable linker (e.g., a linker, or segment thereof, that does not include a trigger element or immolative element).

In some embodiments, R¹, R², or R^{3a} comprises one of the following structures:

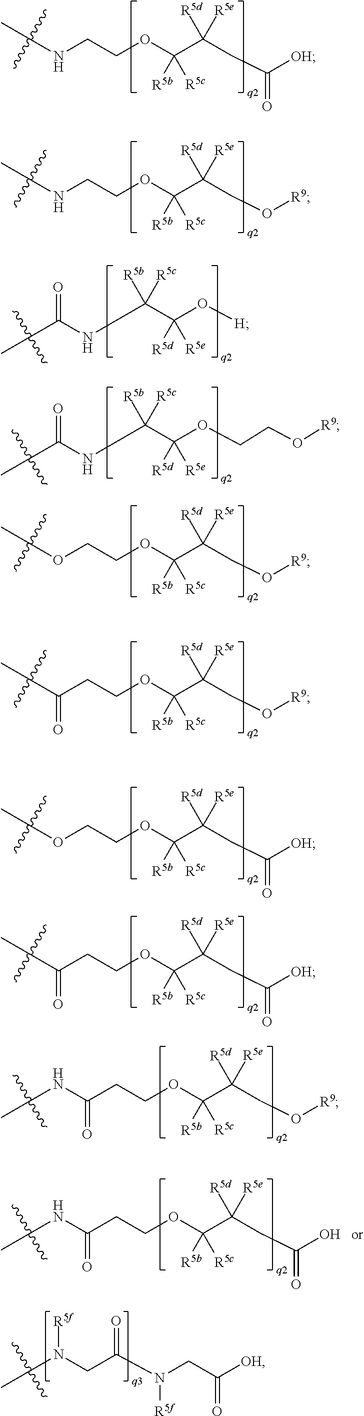

wherein
each occurrence of R^{5b}, R^{5c}, R^{5d} and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide;
each occurrence of R^{5f} is independently hydrogen, alkyl, hydroxyalkyl, or alkoxyalkyl;
each occurrence of R⁹ is independently hydrogen or alkyl;
each occurrence of q2 is independently an integer from 1-25; and
each occurrence of q3 is independently an integer from 5-15.

In some embodiments, a hydrophilic element comprises polyethylene glycol, polysarcosine, cyclodextrin, c-glycosides, or combinations thereof. In some embodiments, a hydrophilic element comprises one of the following structures:

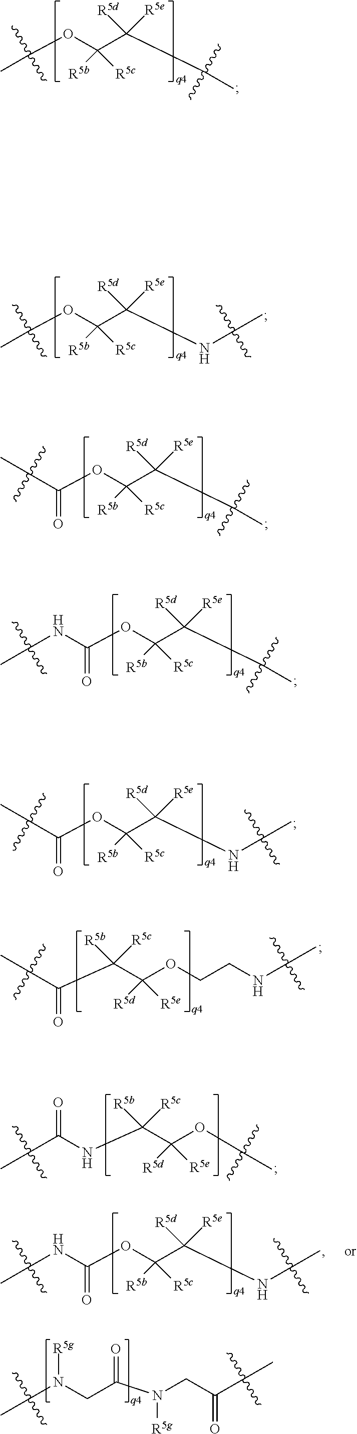

wherein:
   each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
   each occurrence R^{5g} is independently hydrogen, alkyl, hydroxyalkyl, or alkoxyalkyl; and
   each occurrence of q4 is, independently an integer from 1-24.

In some embodiments, a hydrophilic element comprises one of the following structures:

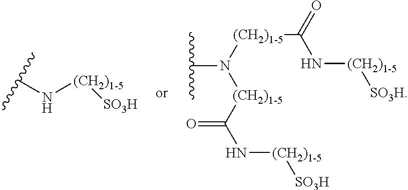 [SMILES: *NCS(=O)(=O)O ; *N(CC(=O)NCS(=O)(=O)O)CC(=O)NCS(=O)(=O)O]

In some embodiments, a hydrophilic element comprises one of the following structures:

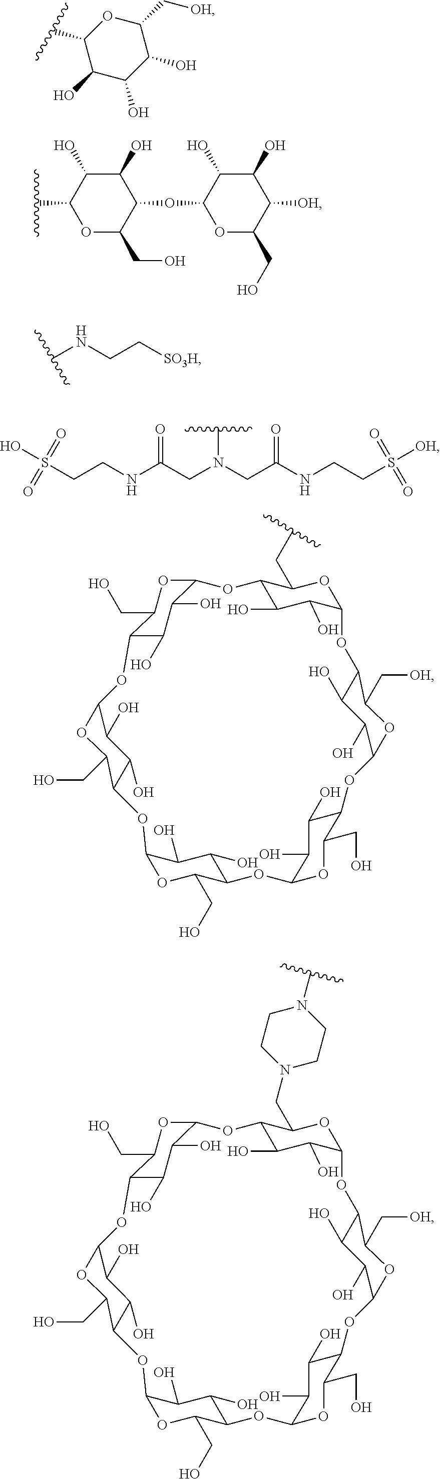

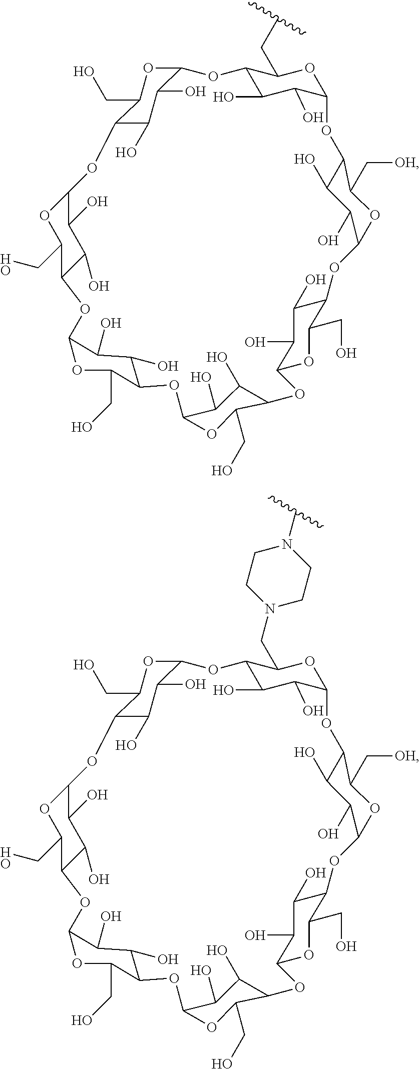 [SMILES: *CC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O ; *N1CCN(CC2OC3OC4C(CO)OC(OC5C(CO)OC(OC6C(CO)OC(OC7C(CO)OC(OC8C(CO)OC(OC9C(CO)OC(OC2C(O)C3O)C(O)C9O)C(O)C8O)C(O)C7O)C(O)C6O)C(O)C5O)C(O)C4O)CC1]

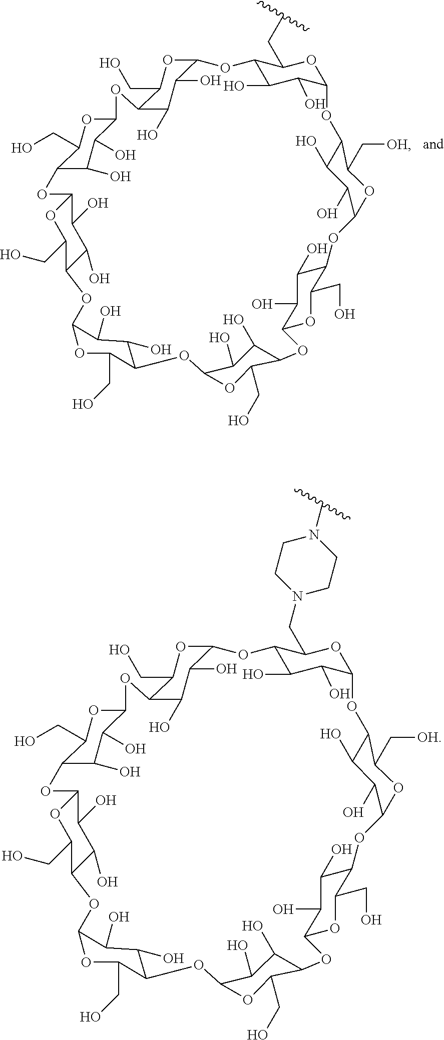 [SMILES: *CC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O ; "and" ; *N1CCN(CC2OC3OC4C(CO)OC(OC5C(CO)OC(OC6C(CO)OC(OC7C(CO)OC(OC8C(CO)OC(OC9C(CO)OC(OC%10C(CO)OC(OC2C(O)C3O)C(O)C%10O)C(O)C9O)C(O)C8O)C(O)C7O)C(O)C6O)C(O)C5O)C(O)C4O)CC1]

In some embodiments, a hydrophilic element has one of the following structures:

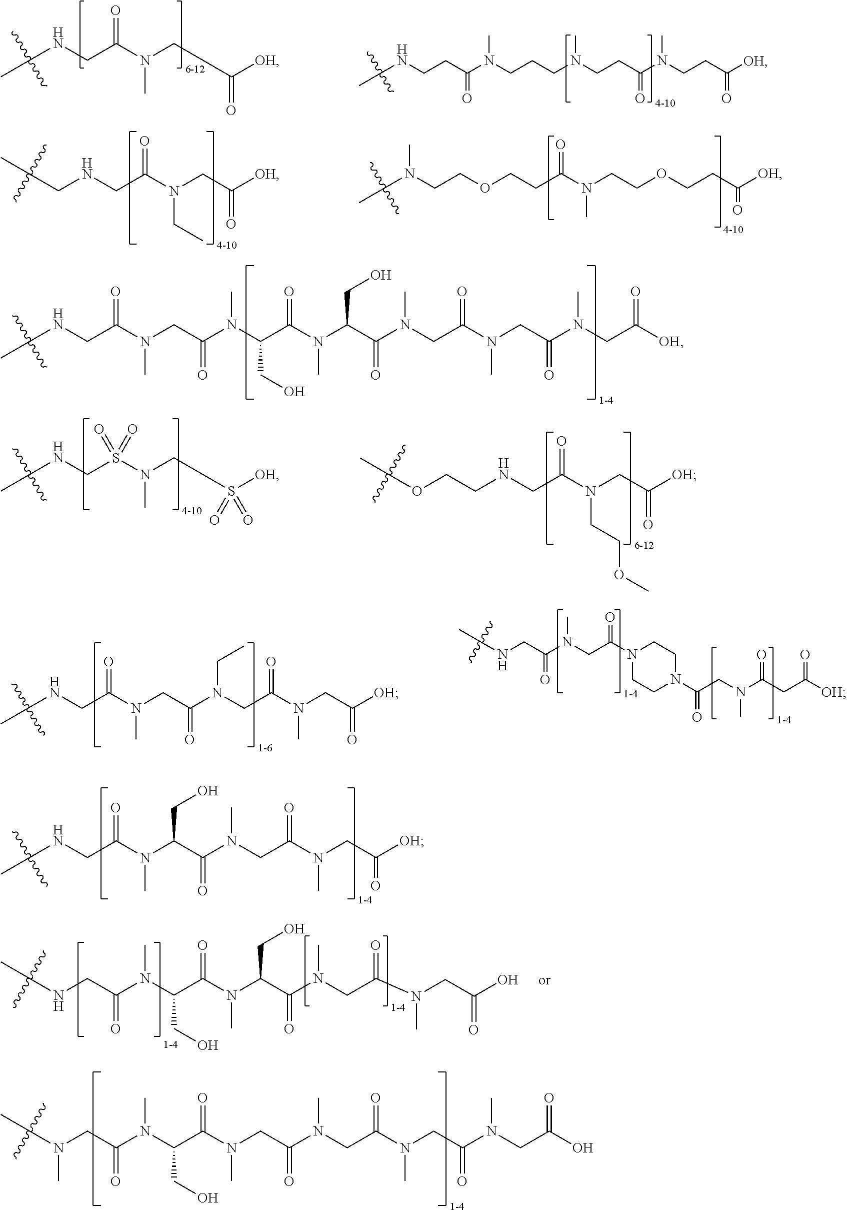

In some embodiments, a hydrophilic element has the following structure:

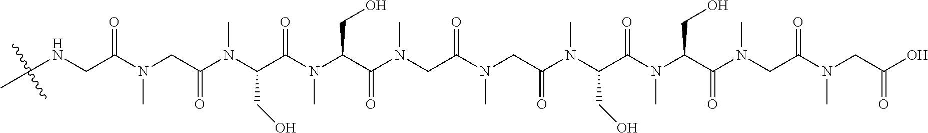 [SMILES: *NCC(=O)N(C)CC(=O)N(C)[C@@H](CO)C(=O)N(C)[C@@H](CO)C(=O)N(C)CC(=O)N(C)CC(=O)N(C)[C@@H](CO)C(=O)N(C)[C@@H](CO)C(=O)N(C)CC(=O)N(C)CC(=O)O]

In some embodiments, a hydrophilic element has one of the following structures:

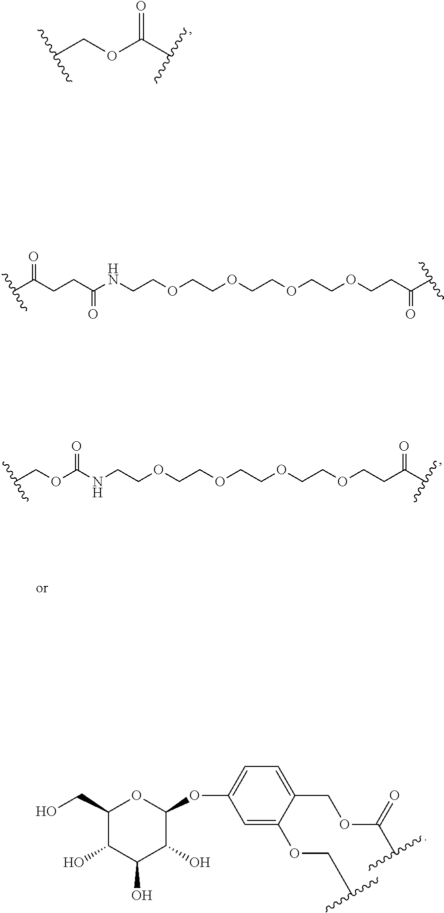 [SMILES: *COC(*)=O ; *C(=O)CCOCCOCCOCCOCCNC(=O)CCC(*)=O ; *COC(=O)NCCOCCOCCOCCOCCC(*)=O ; *COc1cc(O[C@@H]2O[C@H](CO)[C@@H](O)[C@H](O)[C@H]2O)ccc1COC(*)=O]

In some embodiments, a hydrophilic element has one of the following structures:

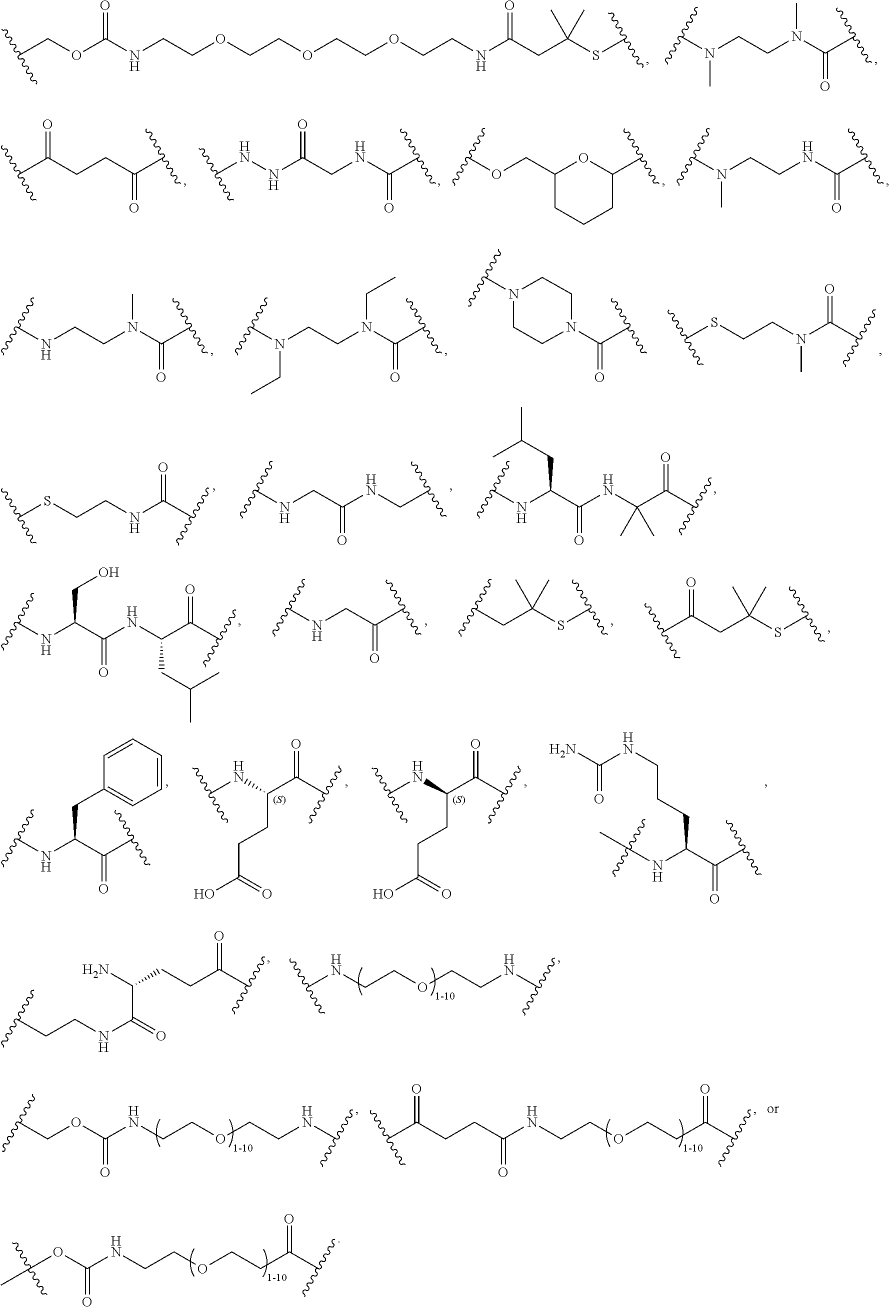

In some embodiments, a hydrophilic element comprises a polysarcosine. In some embodiments, a hydrophilic element is a polysarcosine comprising the following structure:

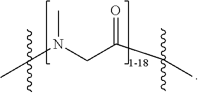

In some embodiments, a hydrophilic element is a polysarcosine with one of the following structures:

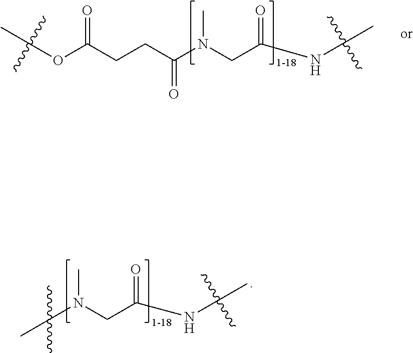

In some embodiments, a hydrophilic element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a hydrophilic element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In certain embodiments, L¹ is alkylene. In some embodiments, L¹ is C₁-C₆ alkylene. In certain embodiments, L² is alkylene. In some embodiments, L² is C₁-C₆ alkylene. In certain embodiments, L³ is alkylene. In some embodiments, L³ is C₁-C₆ alkylene.

In certain embodiments, L¹ is heteroalkylene. In some embodiments, L¹ is C₁-C₆ heteroalkylene (i.e., contains from 1-6 carbon atoms and one or more heteroatoms). In certain embodiments, L² is heteroalkylene. In some embodiments, L² is C₁-C₆ heteroalkylene. In certain embodiments, L³ is heteroalkylene. In some embodiments, L³ is C₁-C₆ heteroalkylene.

In more embodiments, L¹, L², or L³ are C₁-C₆ heteroalkylene and contain heteroatoms selected form 0 and N. In some embodiments, L³ is a direct bond.

In more embodiments, L¹, L², or L³ have one of the following structures:

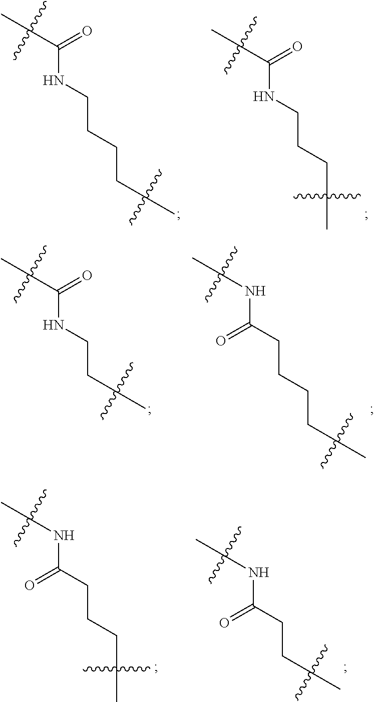 [SMILES: *CCCCNC(*)=O ; *CCCNC(*)=O ; *CCNC(*)=O ; *CCCCC(=O)N* ; *CCCC(=O)N* ; *CCC(=O)N*]

In some embodiments, a trigger element comprises a dipeptide, a tripeptide, a tetrapeptide, a pentapeptide, a glucuronide, a disulfide, a phosphate, a diphosphate, a triphosphate, a hydrazone, or combinations thereof. In some other embodiments, a trigger element comprises beta-glucuronic acid. In certain embodiments, a trigger element comprises a dipeptide, a tripeptide, a tetrapeptide, or a pentapeptide. In some embodiments, a trigger element comprises two or more amino acids selected from the group consisting of valine, citrulline, alanine, glycine, phenylalanine, lysine, or combinations thereof. In certain embodiments, a trigger element comprises a sequence of amino acids selected from the group consisting of valine-citrulline, valine-alanine, glycine-glycine-phenylalanine-glycine, and combinations thereof. In some embodiments, a trigger element comprises one of the following structures, including combinations thereof:

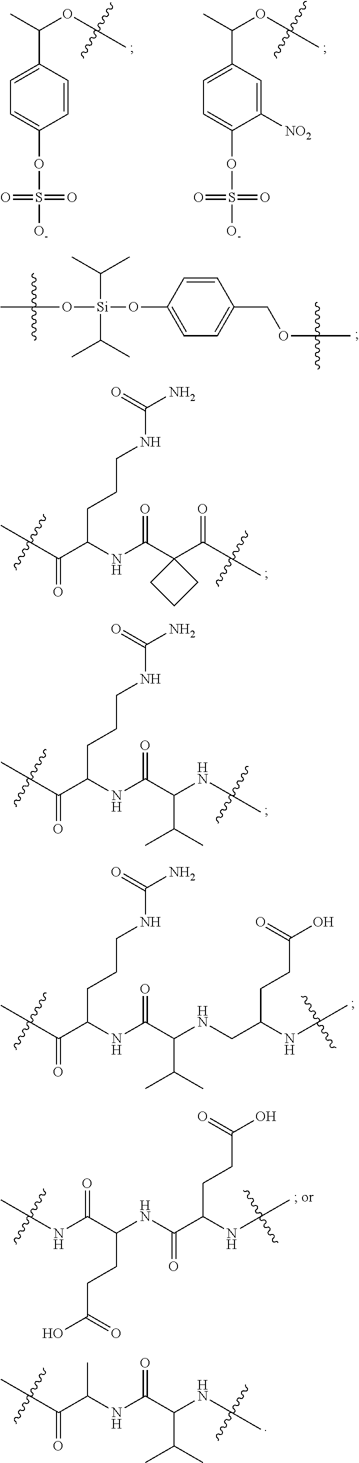

In some embodiments, a trigger element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, a trigger element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, a trigger element has the following structure:

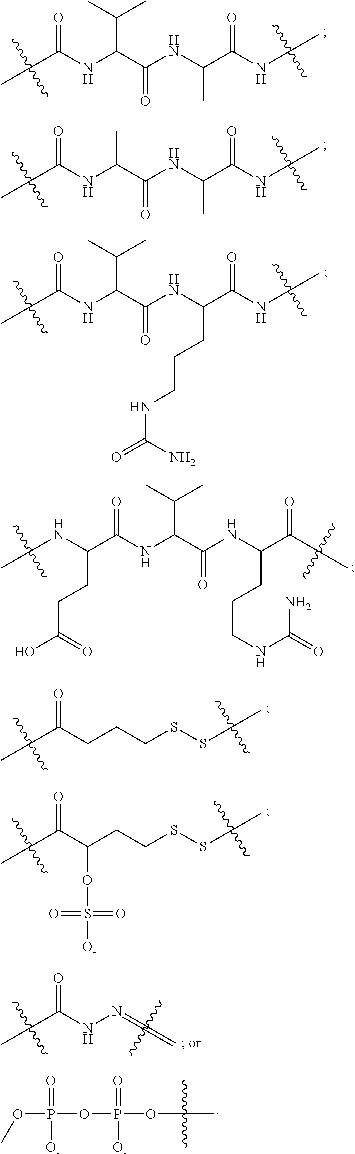

In some embodiments, a trigger element is specifically cleaved by an enzyme. For example, a trigger element can be cleaved by a lysosomal enzyme. A trigger element can be peptide-based or can include peptidic regions that can act as substrates for enzymes. Peptide based trigger elements can be more stable in plasma and extracellular milieu than chemically labile linkers.

Exemplary disulfide-containing trigger elements can include the following structures:

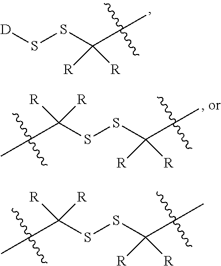

wherein D is a compound of Structure (I) or (II) and R is independently selected at each occurrence from, for example, hydrogen or C₁-C₆ alkyl. Increasing steric hindrance adjacent to the disulfide bond can increase the stability of the linker. The above structures can result in increased in vivo stability when one or more R groups is selected from a lower alkyl, such as methyl.

Peptide bonds can have good serum stability, as lysosomal proteolytic enzymes can have very low activity in blood due to endogenous inhibitors and the unfavorably high pH value of blood compared to lysosomes. Release of a compound of Structure (I) or (II) from conjugate of Structure (IIx) can occur due to the action of lysosomal proteases, e.g., cathepsin and plasmin. These proteases can be present at elevated levels in certain tumor tissues. A trigger element can be cleavable by a lysosomal enzyme. The lysosomal enzyme can be, for example, cathepsin B, β-glucuronidase, or β-galactosidase.

A cleavable peptide of a trigger element can be selected from tetrapeptides such as Gly-Phe-Leu-Gly, Ala-Leu-Ala-Leu, tripeptides such as Glu-Val-Cit, or dipeptides such as Val-Cit, Val-Ala, Ala-Ala, and Phe-Lys. Dipeptides can have lower hydrophobicity compared to longer peptides.

In some embodiments, a trigger element may be a single amino acid residue. In some embodiments, the trigger element comprises Asn (e.g., a legumain cleavable).

Enzymatically cleavable trigger elements be combined with an immolative element to provide additional spatial separation between a compound of Structure (I) or (II) and the site of enzymatic cleavage. The direct attachment of a compound of Structure (I) or (II) to a peptidic trigger element can result in proteolytic release of a compound of Structure (I) or (II) or of an amino acid adduct of a compound of Structure (I) or (II) thereby impairing its activity. The use of an immolative element can allow for the release of the fully active, chemically unmodified compound of Structure (I) or (II) upon amide bond hydrolysis.

A trigger element can contain a chemically labile group such as hydrazone and/or disulfide groups. A trigger element comprising chemically labile group or groups can exploit differential properties between the plasma and some cytoplasmic compartments. The intracellular conditions that can facilitate release of a compound of Structure (I) or (II) for hydrazone containing trigger elements can be the acidic environment of endosomes and lysosomes, while the disulfide containing trigger elements can be reduced in the cytosol, which can contain high thiol concentrations, e.g., glutathione. The plasma stability of a trigger element containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a compound of Structure (I) or (II) once the conjugate of Structure (IIx) is internalized into mildly acidic endosomal (pH 5.0-6.5) and lysosomal (pH 4.5-5.0) compartments of the cell. This pH dependent release mechanism can be associated with non-specific release of a compound of Structure (I) or (II). To increase the stability of a hydrazone group of a trigger element, a trigger element can be varied by chemical modification, e.g., substitution, allowing tuning to achieve more efficient release in the lysosome with a minimized loss in circulation.

In some embodiments, a trigger element comprises a hydrazone moiety having one of the following structures:

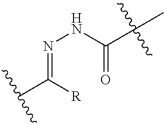

wherein R is selected from C₁-C₆ alkyl, aryl, and —O-ci-C₆ alkyl.

Hydrazone-containing trigger elements can contain additional cleavage sites, such as additional acid-labile cleavage sites and/or enzymatically labile cleavage sites (e.g., a disulfide). Conjugates and compounds including exemplary hydrazone-containing trigger elements can include, for example, the following structure:

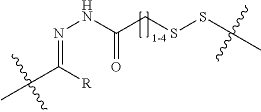

wherein R is selected from C₁-C₆ alkyl, aryl, and —O—C₁-C₆ alkyl.

Other acid-labile groups that can be included in trigger elements include cis-aconityl-containing linkers. cis-Aconityl chemistry can use a carboxylic acid juxtaposed to an amide bond to accelerate amide hydrolysis under acidic conditions.

Trigger elements can also include a disulfide group. Disulfides can be thermodynamically stable at physiological pH and release a compound of Structure (I) or (II) upon internalization of the conjugate of Structure (IIx) into cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing trigger element can be reasonably stable in circulation, selectively releasing a compound of Structure (I) or (II) in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing trigger element can be enhanced by chemical modification of a trigger element, e.g., use of steric hindrance adjacent to the disulfide bond.

A trigger element can also be a ß-glucuronic acid-based linker. Facile release of a compound of Structure (I) or (II), can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of a conjugate to undergo aggregation due to the hydrophilic nature of ß-glucuronides. In some embodiments, a trigger element comprises a ß-glucuronic acid.

The following scheme depicts the release of a compound of Structure (I) or (II) (D) from a conjugate of Structure (IIx) containing a ß-glucuronic acid-based trigger element:

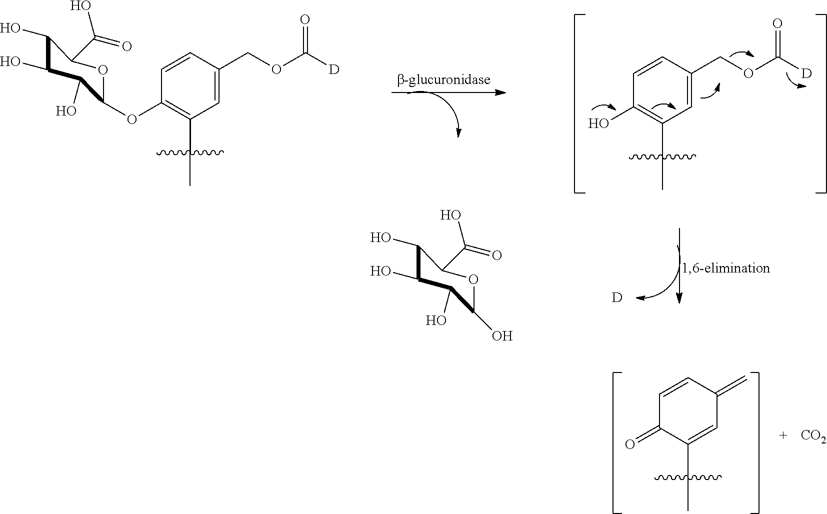

A variety of cleavable β-glucuronic acid-based linkers useful for linking drugs such as auristatins, camptothecin analogues, doxorubicin analogues, CBI minor-groove binders, and psymberin to antibodies have been described. Accordingly, these β-glucuronic acid-based trigger elements are used in the conjugates of Structure (IIx). In some embodiments, a trigger element comprises a β-galactoside-based linker. β-Galactoside is present abundantly within lysosomes, while the enzyme activity outside cells is low.

A trigger element may include one or more peptides. In some embodiments, a peptide can be selected to contain natural amino acids, unnatural amino acids, or any combination thereof. In some embodiments, a peptide can be a tripeptide or a dipeptide. In particular embodiments, a dipeptide comprises L-amino acids, such as Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Trigger elements and immolative groups are known in the art, for example in International Application No. PCT/US2021/054296; the trigger elements and immolative elements of which are hereby incorporated by reference in their entirety.

One immolative element can be a bifunctional para-aminobenzyl alcohol group, which can link to a trigger element through an amino group, forming an amide bond, while an amine containing compound of Structure (I) or (II) can be attached through carbamate functionalities to the benzylic hydroxyl group of thepara-aminobenzyl alcohol (to give ap-amidobenzylcarbamate. The resulting pro-compound can be activated upon protease-mediated cleavage, leading to a 1,6-elimination reaction releasing the unmodified compound of Structure (I) or (II) and remnants of the antibody-linker.

In some embodiments, an immolative element comprises para-aminobenzyloxycarbonyl, an aminal, a hydrazine, a disulfide, an amide, an ester, a hydrazine, a phosphotriester, a diester, a β-glucuronide, a double bond, a triple bond, an ether bond, a ketone, a diol, a cyano, a nitro, a quaternary amine, or combinations thereof. In certain embodiments, an immolative element comprises a paramethoxybenzyl, a dialkyldialkoxysilane, a diaryldialkoxysilane, an orthoester, an acetal, an optionally substituted β-thiopropionate, a ketal, a phosphoramidate, a hydrazone, a vinyl ether, an imine, an aconityl, a trityl, a polyketal, a bis-arylhydrazone, a diazobenzene, a vivinal diol, a pyrophosphate diester, or combinations thereof.

In certain embodiments, an immolative element comprises one of the following structures:

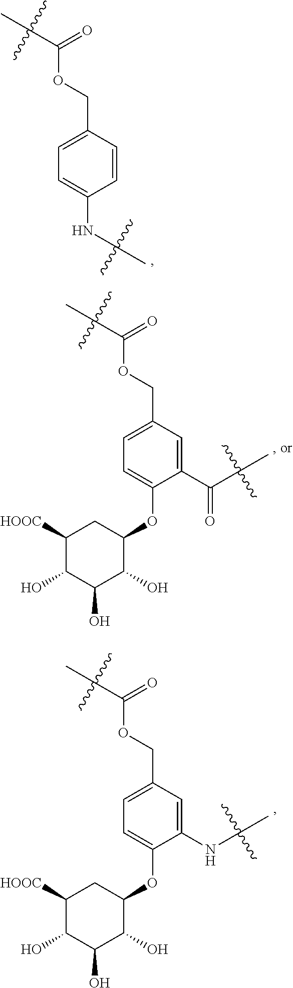 [SMILES: *Nc1ccc(COC(*)=O)cc1 ; *C(=O)OCc1ccc(O[C@@H]2C[C@H](C(=O)O)[C@@H](O)[C@H](O)[C@H]2O)c(C(*)=O)c1 ; *Nc1cc(COC(*)=O)ccc1O[C@@H]1C[C@H](C(=O)O)[C@@H](O)[C@H](O)[C@H]1O]

In some embodiments, an immolative element comprises the following structure:

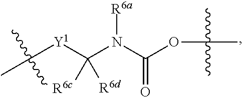

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6b} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—.

In certain embodiments, an immolative element comprises the following structure:

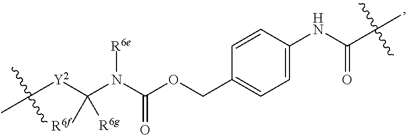

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—.

In some embodiments, an immolative element comprises the following structure:

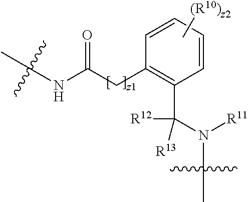

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10.

In some embodiments, an immolative element comprises one of the following structures:

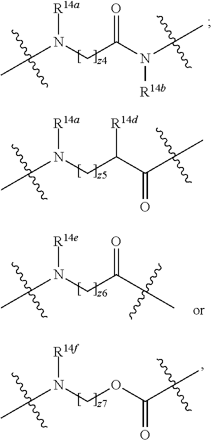

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6.

In some embodiments, an immolative element comprises one of the following structures:

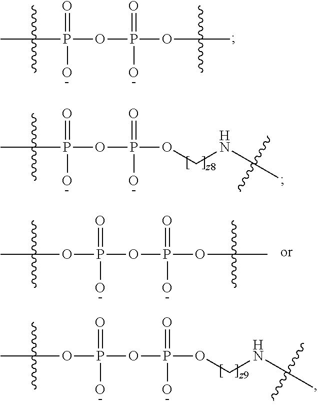

wherein:
z8 and z9 are each independently 1, 2, 3, 4, 5, or 6.

In certain embodiments, an immolative element comprises one of the following structures:

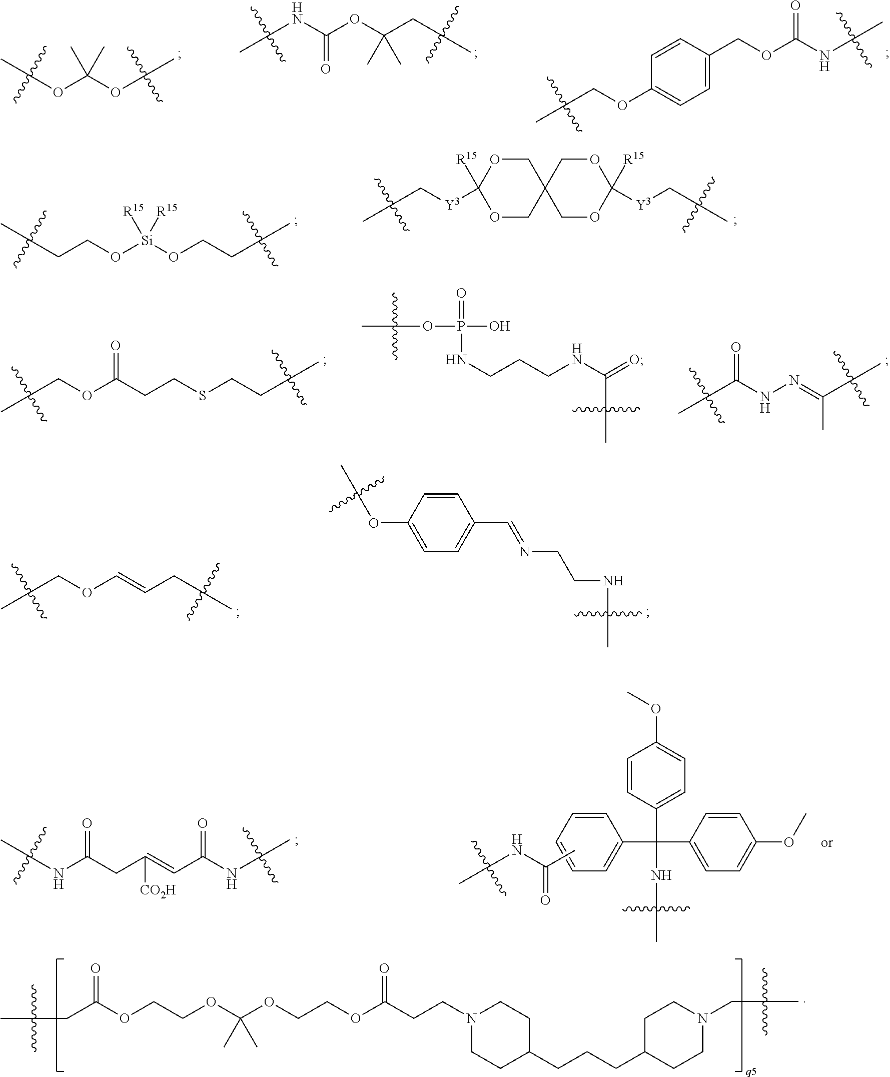

wherein:
each occurrence of R¹⁵ is independently H, methyl, ethyl, isopropyl, tert-butyl, or phenyl;
Y³ is O or CH₂; and
q5 is an integer ranging from 1-5.

In some embodiments, an immolative element has a molecular weight greater than 150 g/mole, greater than 200 g/mole, greater than 300 g/mole, greater than 400 g/mole, greater than 500 g/mole, greater than 600 g/mole, greater than 700 g/mole, greater than 800 g/mole, greater than 900 g/mole, or greater than 1000 g/mole. In some embodiments, an immolative element has a molecular weight less than 150 g/mole, less than 200 g/mole, less than 300 g/mole, less than 400 g/mole, less than 500 g/mole, less than 600 g/mole, less than 700 g/mole, less than 800 g/mole, less than 900 g/mole, or less than 1000 g/mole.

In some embodiments, an immolative element and a trigger element together have the following structure:

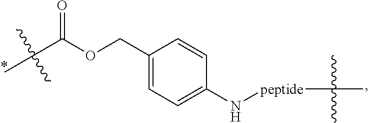

wherein a trigger element is denoted with “peptide” and comprises from one to ten amino acids, and [symbol] represents the point of attachment to a compound of Structure (I) or (II). In some embodiments, the peptide comprises Val-Cit or Val-Ala. Heterocyclic variants (e.g., pyridinyl, pyrimidinyl, etc.) of this immolative element may also be used.

In some embodiments, an immolative element contains a phenol group that is covalently bound to the remainder of the molecule through the phenolic oxygen. One such immolative element relies on a methodology in which a diamino-ethane “Space Link” is used in conjunction with traditional “PABO”-based immolative element to deliver phenols.

In some embodiments, a trigger element can include non-cleavable portions or segments. Polyethylene glycol (PEG) and related polymers can be included with cleavable groups such as a disulfide, a hydrazone or a dipeptide to form an immolative group and/or trigger element.

Other degradable linkages that can be included in immolative elements can include esters. Esters can be formed by the reaction of PEG carboxylic acids or activated PEG carboxylic acids with alcohol groups on a compound of Structure (I) or (II) such ester groups can hydrolyze under physiological conditions to release a compound of Structure (I) or (II). Other hydrolytically degradable linkages can include carbonate linkages, imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including at the end of a polymer, and a 5′ hydroxyl group of an oligonucleotide.

In some embodiments, a trigger element, immolative group, and compound of Structure I or II (depicted as “Payload”) together have the following structure:

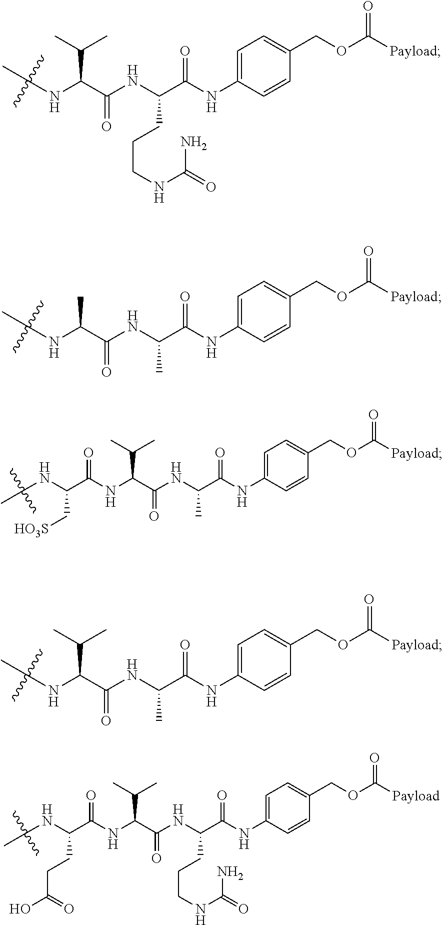

wherein [symbol] indicates an attachment site to the remainder of the molecule (i.e., a linker of Structure (Ix) or conjugate of Structure (IIx)) and a compound of Structure (I) or (II) is indicated with text (i.e., “payload”). Amino acids of embodiments above may be replaced or used in addition to other amino acids, in some embodiments, a trigger element is Asn-Cit, Arg-Cit, Val-Glu, Ser-Cit, Lys-Cit, Asp-Cit, Phe-Lys, Glu-Val-Cit, Glu-Val-Cit, Glu-Glu-Val-Cit, or Glu-Glu-Glu-Val-Cit, and an immolative element is PABC.

In some embodiments, the phenyl portion of the PABC is substituted with one or more substituents. In some embodiments, the substituents have one of the following structures:

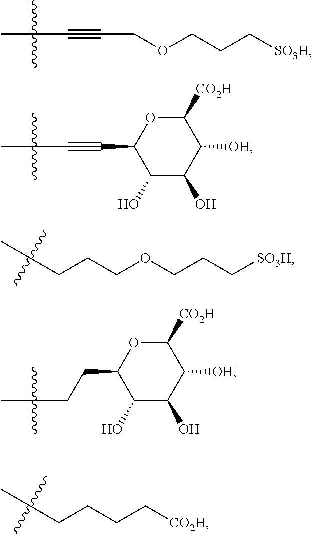

In some embodiments, an immolative group comprises one of the following structures:

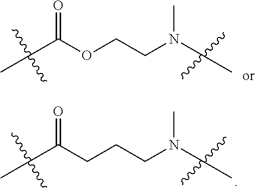

In some embodiments, a trigger element, an immolative element, and a compound of Structure (I) or (II) (represented as “Payload”) together have one of the following structures:

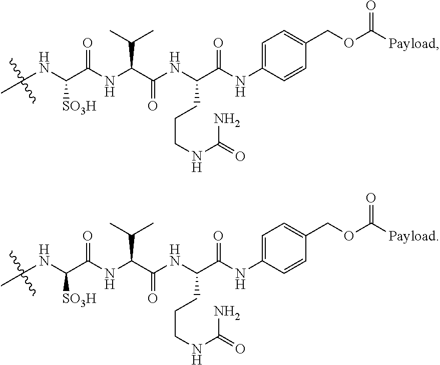

In some embodiments, an immolative element has the following structure:

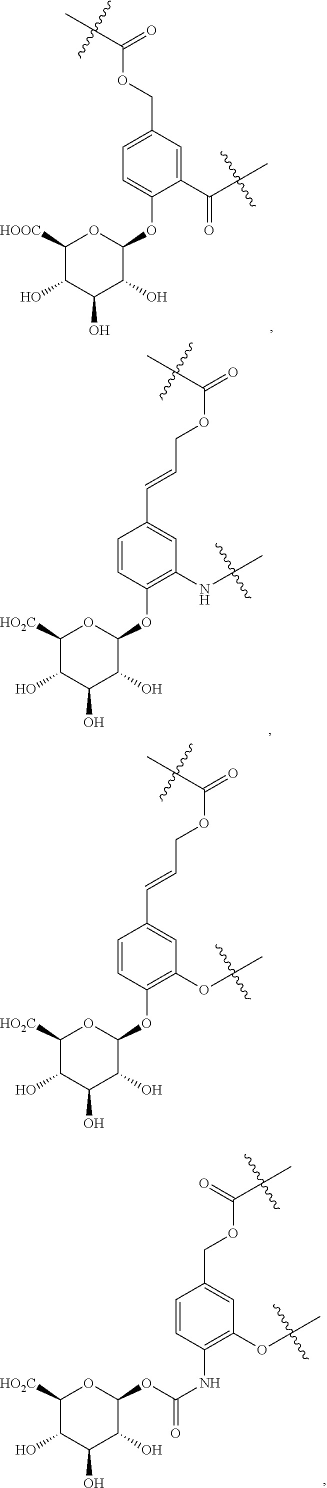

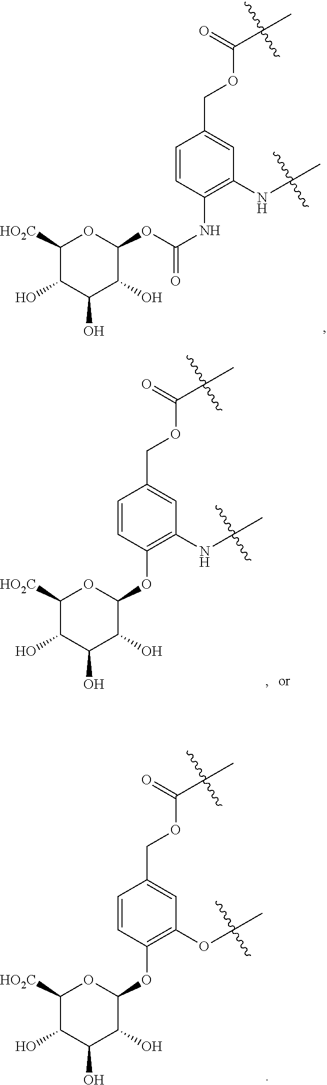

The structures above show a substitution pattern of 1, 3, 4 on the phenyl ring of an immolative element. In some embodiments, a substitution pattern may be 1, 2, 4 (i.e., 1 being a linkage to a compound of Structure (I) or (II), 2 being a linkage to the remainder of the molecule and 4 being a linkage to the carbohydrate) or 1, 3, 5 (i.e., 1 being a linkage to a compound of Structure (I) or (II), 3 being a linkage to the remainder of the molecule and 4 being a linkage to the carbohydrate).

Although cleavable linker (e.g., linkers with trigger elements or immolative elements) can provide certain advantages, linkers need not be cleavable. For non-cleavable linkers, a compound of Structure (I) or (II) release may not depend on the differential properties between the plasma and some cytoplasmic compartments. The release of a compound of Structure (I) or (II) can occur after internalization of the conjugate of Structure (IIx) via antigen-mediated endocytosis and delivery to lysosomal compartment, where the targeting moiety (or binding fragment thereof) can be degraded to the level of amino acids through intracellular proteolytic degradation. This process can release a compound of Structure (I) or (II) or compound of Structure (I) or (II) derivative. A compound of Structure (I) or (II) or compound of Structure (I) or (II) derivative can be more hydrophilic and less membrane permeable, which can lead to less bystander effects and less non-specific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units. In some embodiments, L¹-R¹ or L²-R² comprises a linker that is non-cleavable in vivo.

In some embodiments, a trigger element and an immolative element together comprise one of the following structures:

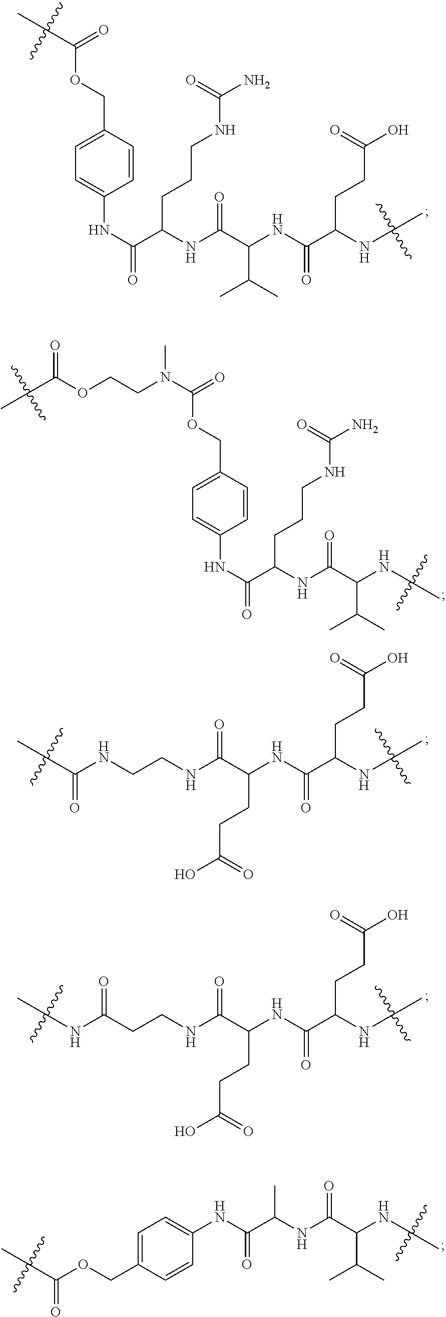

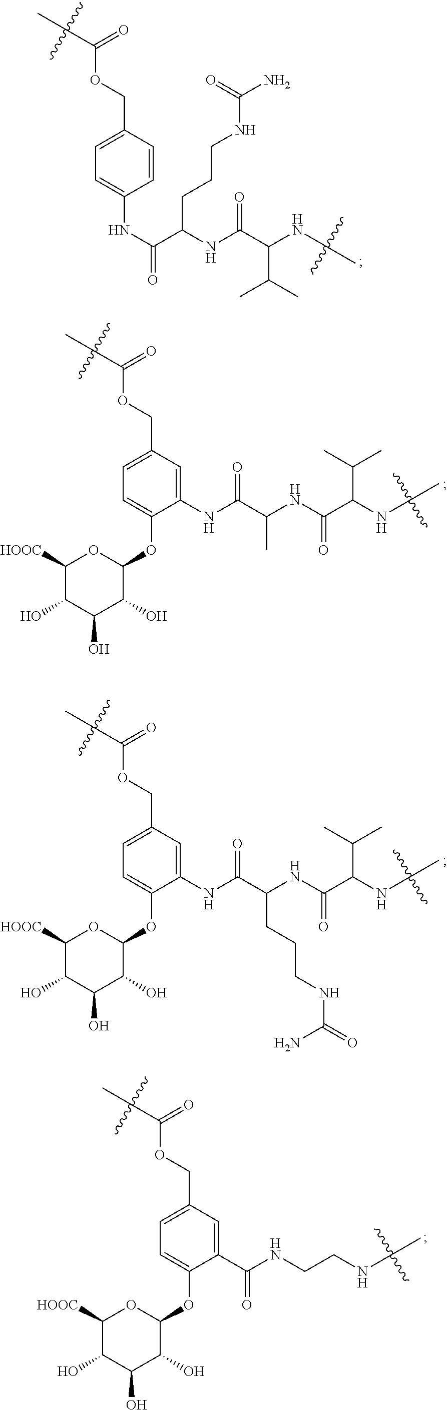 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

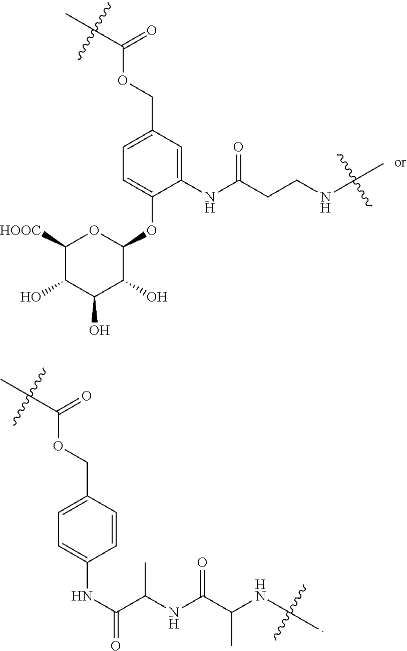 [SMILES: *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, a heteroalkylene element comprises polyethylene glycol or polypropylene glycol. In some embodiments, a heteroalkylene element comprises one of the following structures:

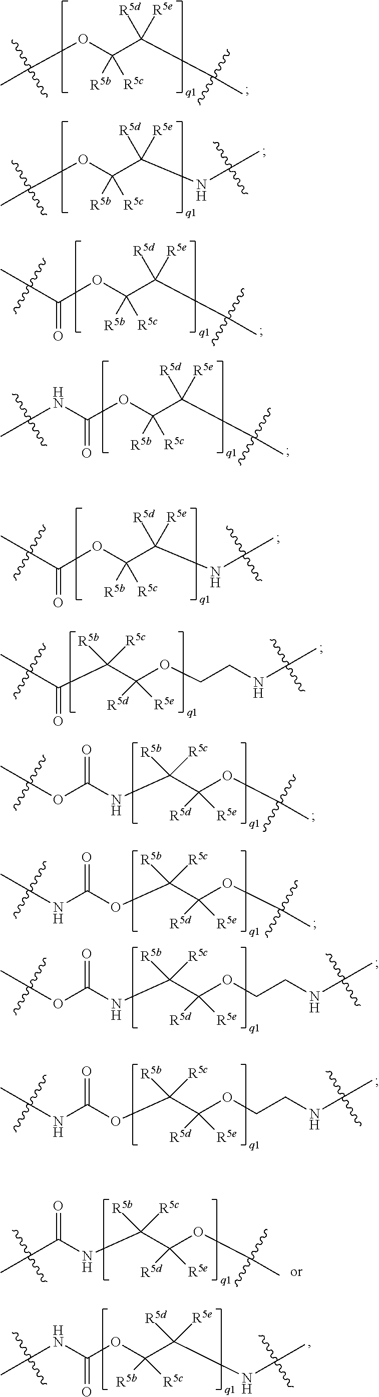

wherein:
each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24.

In some embodiments, R^{5b}, R^{5c}, R^{5d}, and R^{5e} are all hydrogen.

In some embodiments, a polar cap comprises one or more charged amino acid, one or more polyol, or combinations thereof. In certain embodiments, a polar cap comprises a diol, a triol, a tetraol, or combinations thereof. In some embodiments, a polar cap comprises glycerol, trimethylolpropane, pentaerythritol, maltitol, sorbitol, xylitol, erythritol, isomalt, or combinations thereof. In certain embodiments, a polar cap comprises one or more natural amino acids. In some embodiments, a polar cap comprises one or more non-natural amino acids. In some embodiments, a polar cap comprises one or more non-natural amino acids and one or more natural amino acids. In certain embodiments, a polar cap comprises serine, threonine, cysteine, proline, asparagine, glutamine, lysine, arginine, histidine, aspartate, glutamate, 4-hydroxyproline, 5-hydroxylysine, homoserine, homocysteine, ornithine, beta-alanine, statine, or gamma aminobutyric acid. In certain embodiments, a polar cap comprises aspartic acid, serine, glutamic acid, serine-beta-glucose, or combinations thereof.

In some embodiments, a polar cap comprises one of the following structures:

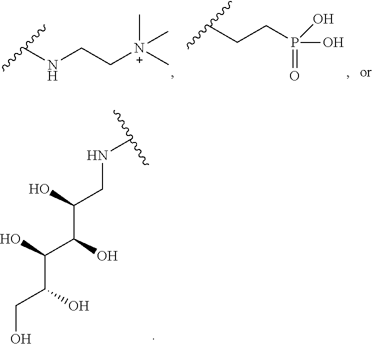

In more embodiments, a polar cap has one of the following structures, including combinations thereof:

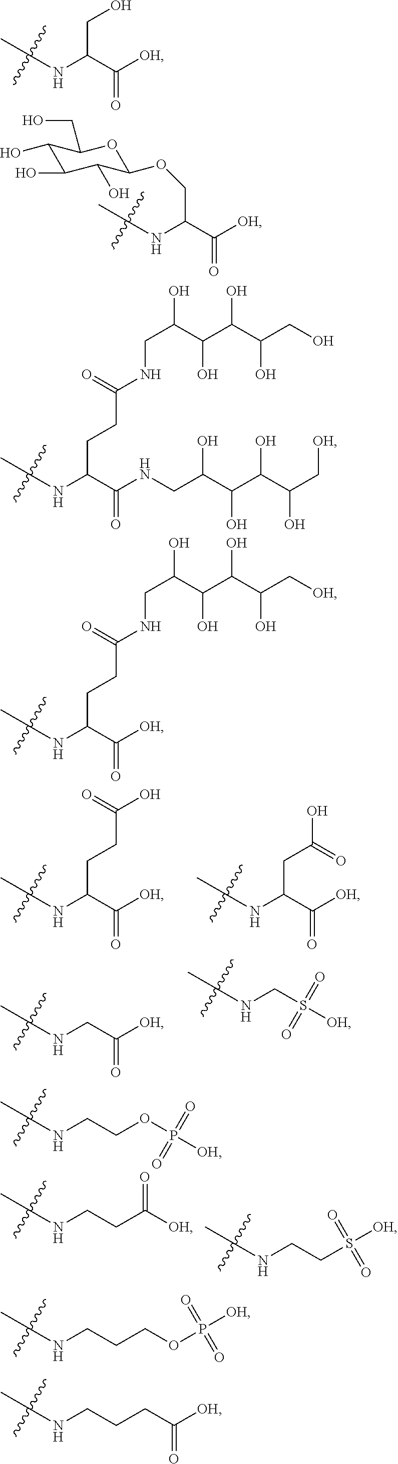

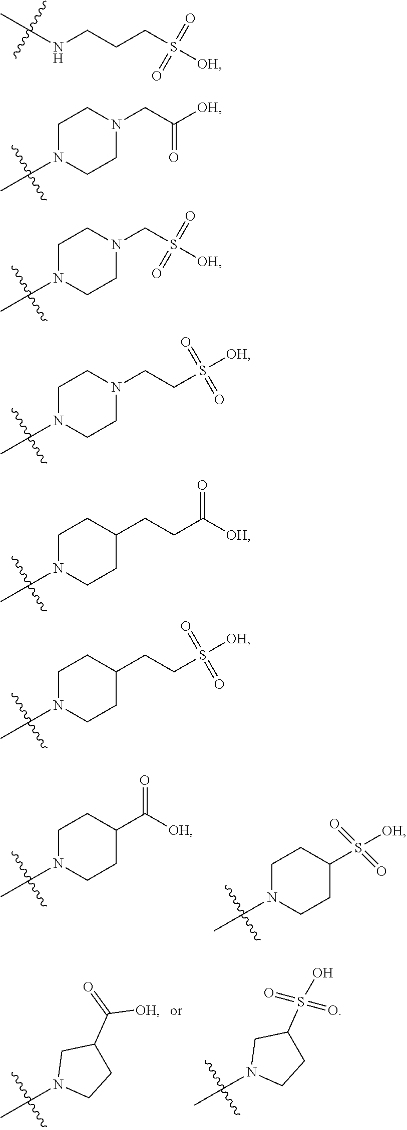

In more embodiments, L¹, L², or L³ comprise a linker selected from the group alkylene, alkylene-L^{a}-, alkenylene, alkenylene-L^{a}-, alkynylene, alkynylene-L^{a}-, -L^{a}-, -L^{a}-alkylene-L^{a}-, -L^{a}-alkenylene-L^{a}-, -L^{a}-alkynylene-L^{a}-, and combinations thereof, wherein each alkylene, alkenylene, and alkynylene is optionally substituted and each occurrence of L^{a} is independently selected from —O—, —S—, —N(R⁷)—, —C(O)—, —C(S)—, —C(O)O—, —OC(O)—, —OC(O)O—, —C(O)N(R⁷)—, —N(R⁷)C(O)—, —C(O)N(R⁷)C(O)—, —C(O)N(R⁷)C(O)N(R⁷), —N(R⁷)C(O)N(R⁷)—, —N(R⁷)C(O)O—, —OC(O)N(R⁷)—, —C(NR⁷)—, —N(R⁷)C(NR⁷)—, —C(NR⁷)N(R⁷)—, —N(R⁷)C(NR⁷)N(R⁷)—, —S(O)₂—, —OS(O)—, —S(O)O—, —S(O), —OS(O)₂—, —S(O)₂O, —N(R⁷)S(O)₂—, —S(O)₂N(R⁷)—, —N(R⁷)S(O)—, —S(O)N(R⁷)—, —N(R⁷)S(O)₂N(R⁷)—, and —N(R⁷)S(O)N(R⁷)— and R⁷ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C3-12 cycloalkyl, and 3- to 12-membered heterocycle, each of which is optionally substituted with one or more substituents independently selected from halo, hydroxyl, cyano, nitro, amino, oxo, thioxo, —C(O)OCH₂C₆H5, —NHC(O)OCH₂C₆H₅, C₁₋₁₀ alkyl, C₁₋₁₀ haloalkyl, C₁₋₁₀ alkoxy, C₂₋₁₀ alkenyl, C₂₋₁₀ alkynyl, C₃₋₁₂ cycloalkyl, and 3- to 12-membered heterocyclyl.

In some embodiments, each L¹, L², or L³ is optionally substituted with one or more substituents selected from alkyl, alkenyl, alkynyl, halo, hydroxyl, cyano, —OR⁸, —SR⁸, amino, aminyl, amido, cycloalkyl, aryl, heterocyclyl, heteroaryl, cycloclkylalkyl, arylalkyl, heterocyclylalkyl, heteroarylalkyl, —C(O)R⁸, —C(O)N(R⁸)₂, —N(R⁸)C(O)R⁸, —C(O)OR⁸, —OC(O)R⁸, —S(O)R⁸, —S(O)₂R⁸, —P(O)(OR⁸)₂, —OP(O)(OR⁸)₂, nitro, oxo, thioxo, ═N(R⁸), or cyano, and R⁸ is hydrogen, alkyl, alkenyl, alkynyl, cycloalkyl, aryl, heterocyclyl, heteroaryl, cycloclkylalkyl, arylalkyl, heterocyclylalkyl, or heteroarylalkyl.

In some embodiments, L¹, L², or L³ are independently selected from the following structures:

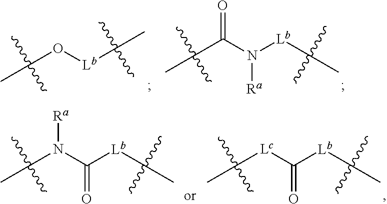

wherein:
   R^{a} is hydrogen or alkyl;
   each occurrence of L^{b} is independently a direct bond, an optionally substituted alkylene linker, an optionally substituted heteroalkylene linker, a heteroatomic linker, or a combination thereof, and each occurrence of L^{c} is independently an optionally substituted alkylene linker and provided that at least one of L¹, L², or L³ has the following structure:

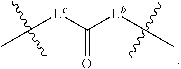

In some embodiments, L¹ and L² are independently selected from the following structures:

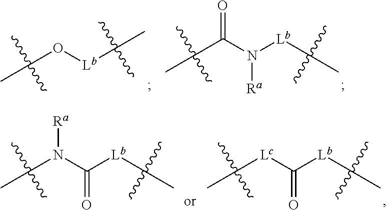

wherein:
R^{a} is hydrogen or alkyl;
each occurrence of L^{b} is independently a direct bond, an optionally substituted alkylene linker, an optionally substituted heteroalkylene linker, a heteroatomic linker, or a combination thereof;
each occurrence of L^{c} is independently an optionally substituted alkylene linker;
provided that at least one of L¹ or L² has the following structure:

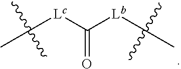

In more embodiments, L² has the following structure:

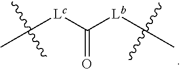

In some embodiments, L^{c} is unsubstituted. In some embodiments, L^{c} is a C₁-C₆ alkylene. In some more embodiments, L^{c} is a C2-C4 alkylene. In some embodiments, L^{c} is a straight C₁-C₆ alkylene. In more embodiments, L^{c} is a straight, unsubstituted C₁-C₆ alkylene. In more embodiments, L^{c} is a straight, unsubstituted C2-C4 alkylene.

In more embodiments, L¹, L², and L³ each independently have one of the following structures:

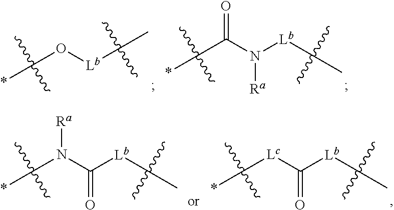

wherein:
* indicates a direct bond to a substitutable position on the phenyl group of following structure:

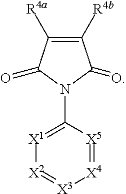

In some embodiments, L¹ or L² has one of the following structures:

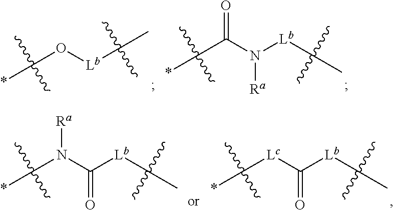

wherein:
* indicates a direct bond to a substitutable position on the phenyl group of following structure:

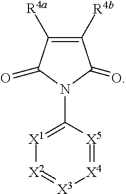

In some embodiments, L^{c} is substituted with one or more substituents selected from the group consisting of halo, haloalkyl, alkoxy, cyano, nitro, carboxy, sulfonamide, sulfonic acid, or combinations thereof.

In some embodiments, the compound has one of the following Structures (Ia) or (Ib):

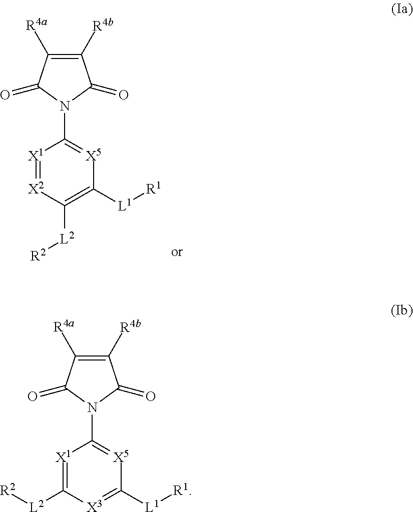

In some embodiments, the compound has one of the following Structures (Ic′) or (Ic″):

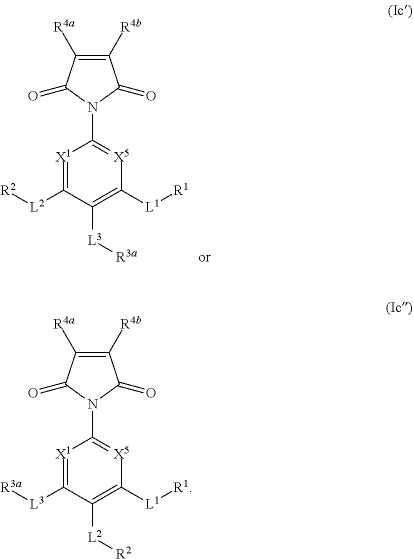

In some embodiments, X¹ is C—F. In certain embodiments, X⁵ is C—F. In certain embodiments, X², X³, or both are C—H, or C—F. In some embodiments, X¹, X⁵, or both are C—R³ and R³ is H or halo. In some embodiments, X¹ and X⁵ are both C—R³ and R³ is H or halo. In some embodiments, X¹ is C—R³ and R³ is halo. In certain embodiments, X⁵ is C—R³ and R³ is halo. In some embodiments, halo is fluoro. In some embodiments, X¹ is C—F. In some embodiments, X¹ is C—H. In some embodiments, X⁵ is C—H. In some embodiments, X⁵ is C—F. In some embodiments, X³ is C—R³ and R³ is H. In some embodiments, X³ is C—R³ and R³ is halo (e.g., fluoro).

In some embodiments, the compound has one of the following structures (Ia-1), (Ia-2), (Ia-3), (Ia-4), (Ia-5), or (Ia-6):

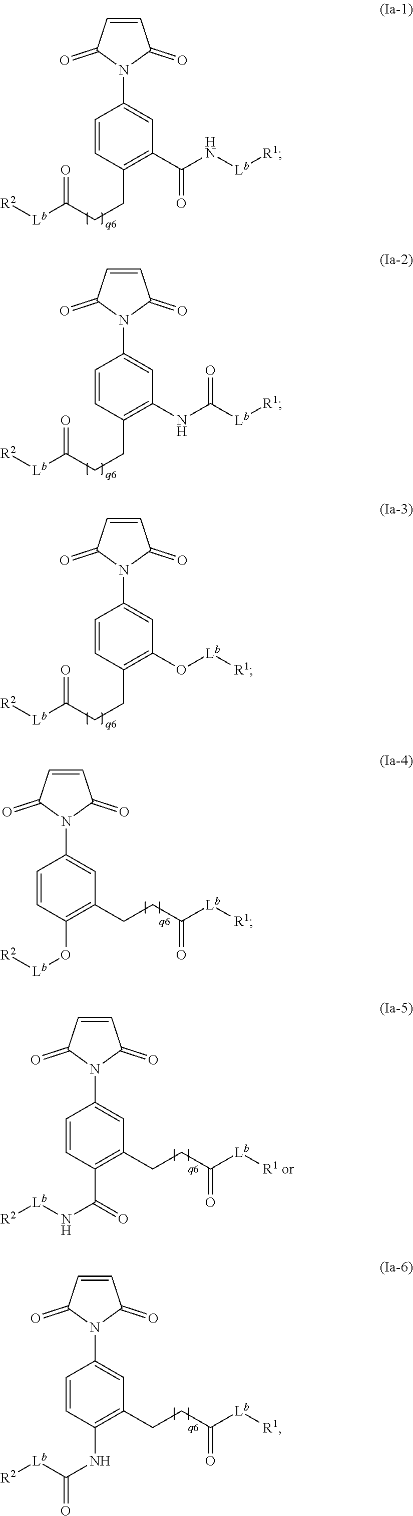

wherein:
   each occurrence of L^{b} is independently a direct bond, an optionally substituted alkylene linker, an optionally substituted heteroalkylene linker, a heteroatomic linker, or combinations thereof, and
   q6 is 0, 1, or 2.

In some embodiments, the compound has one of the following structures (Ia-1), (Ia-2), (Ia-3), (Ia-4), (Ia-5), or (Ia-6):

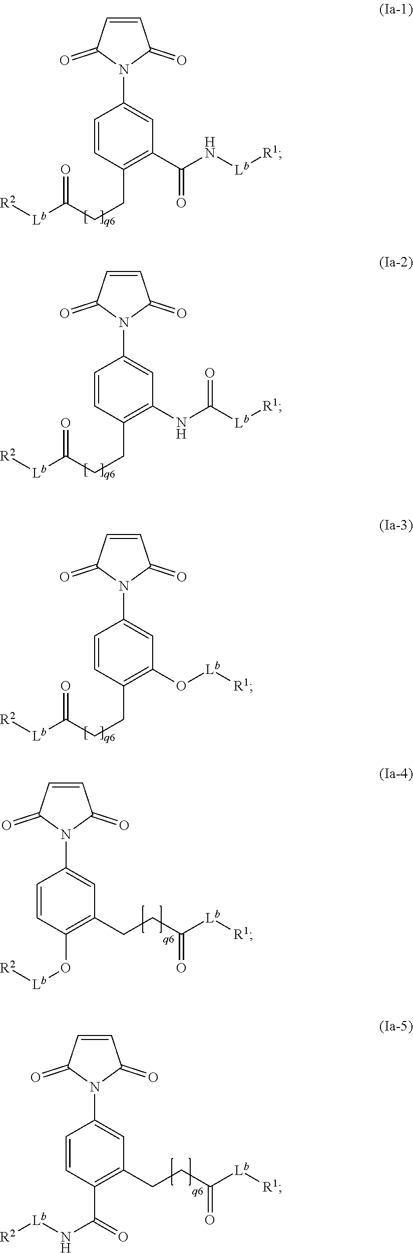

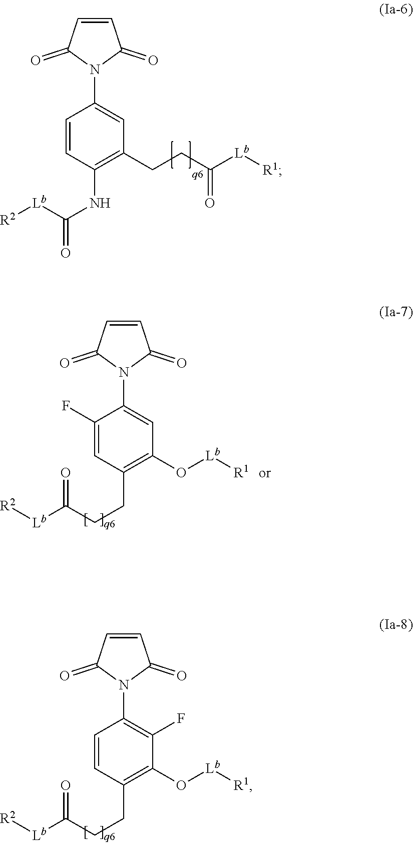

wherein:
   each occurrence of L^{b} is independently a direct bond, an optionally substituted alkylene linker, an optionally substituted heteroalkylene linker, a heteroatomic linker, or combinations thereof, and
q6 is 0, 1, or 2.

In some embodiments, q6 is 1 and L^{b} is gly-gly.

In certain embodiments, the compound has one of the following Structures (Ic-1), (Ic-2), (Ic-3), or (Ic-4):

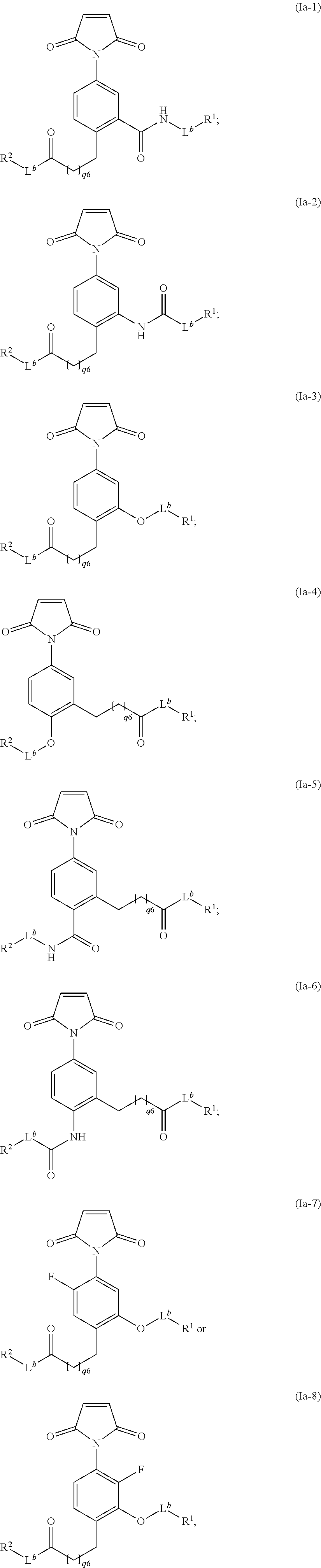

wherein:
   each occurrence of L^{b} is independently a direct bond, an optionally substituted alkylene linker, an optionally substituted heteroalkylene linker, a heteroatomic linker, or combinations thereof, and
q7 is 1, 2, or 3.

In some embodiments, q7 is 2.

In some embodiments, the compound has the following Structure (Id), (Ie), (If), or (Ig):

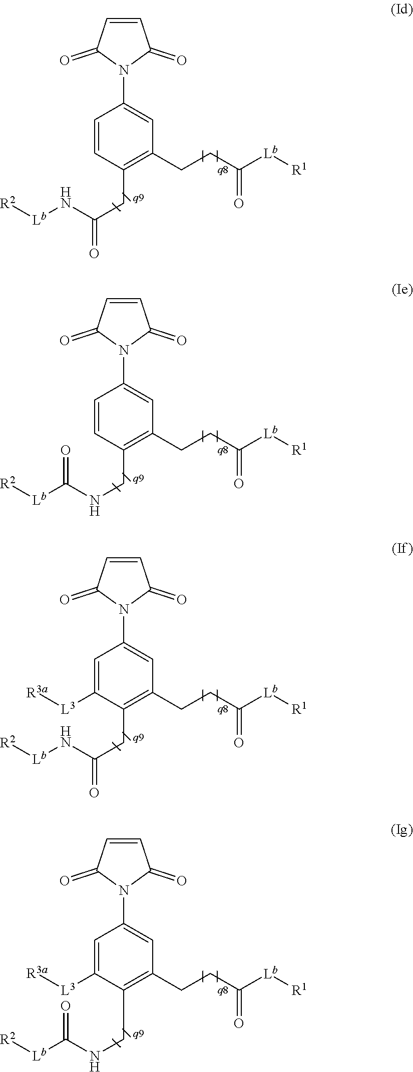

wherein:
each occurrence of L^{b} is independently a direct bond, an optionally substituted alkylene linker,
an optionally substituted heteroalkylene linker, a heteroatomic linker, or combinations thereof,
q8 is 0, 1, or 2; and
q9 is 0, 1, or 2.

In some embodiments, q9 is 0 and q8 is 1.

In some embodiments, the compound has one of the following Structures (Ih) or (Ii):

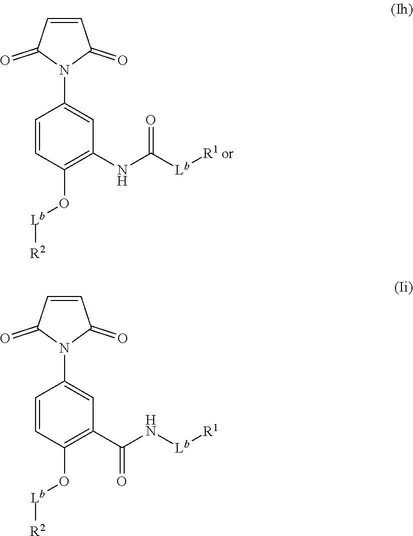

wherein:
each occurrence of L^{b} is independently a direct bond, an optionally substituted alkylene linker, an optionally substituted heteroalkylene linker, a heteroatomic linker, or combinations thereof.

In some embodiments, L^{b} is a direct bond, an optionally substituted alkylene linker or an optionally substituted heteroalkylene linker.

In some embodiments, L^{b} is a direct bond or has one of the following structures:

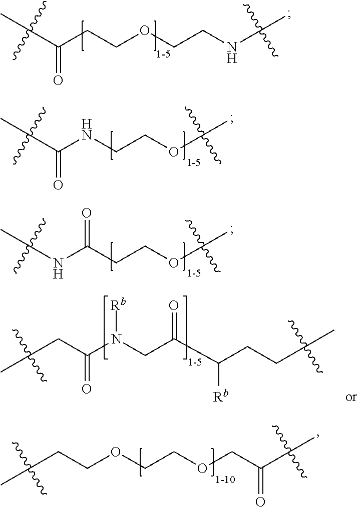

wherein:
each occurrence of R^{b} is independently hydrogen, alkyl, hydroxyalkyl, or alkoxyalkyl.

In some embodiments, each occurrence of R^{b} is —CH₃.

In some embodiments, L¹ or L² has the following structure:

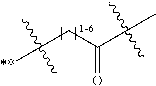

wherein:
** shows a bond to X¹, X², X³, X⁴ or X⁵.

In certain embodiments, X² is C-L¹-R¹ and X³ is C-L²-R². In some embodiments, X³ is C-L¹-R¹ and X² is C-L²-R². In some embodiments, X¹, X⁴, and X⁵ are all CR³. In some embodiments, X¹, X⁴, and X⁵ are all CH.

In some embodiments, the compound has one of the following structures:

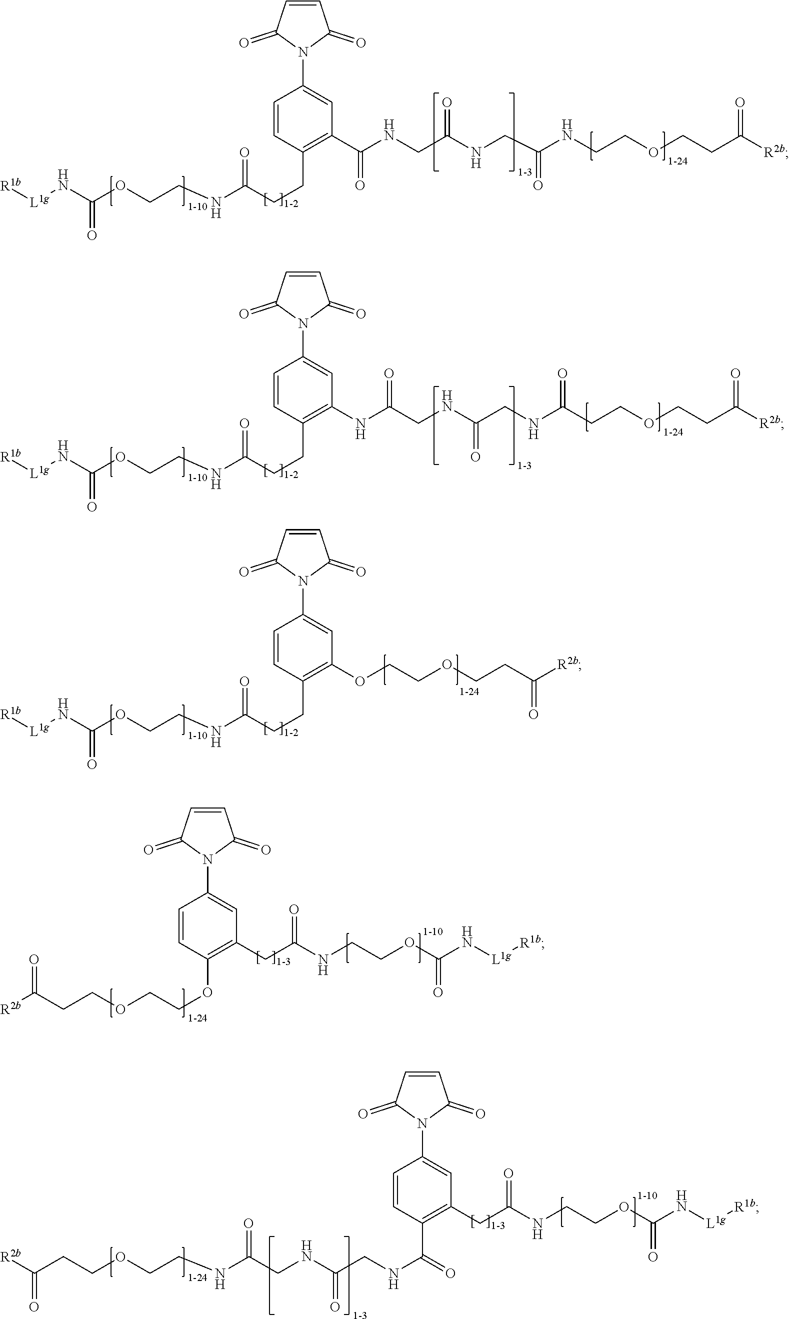

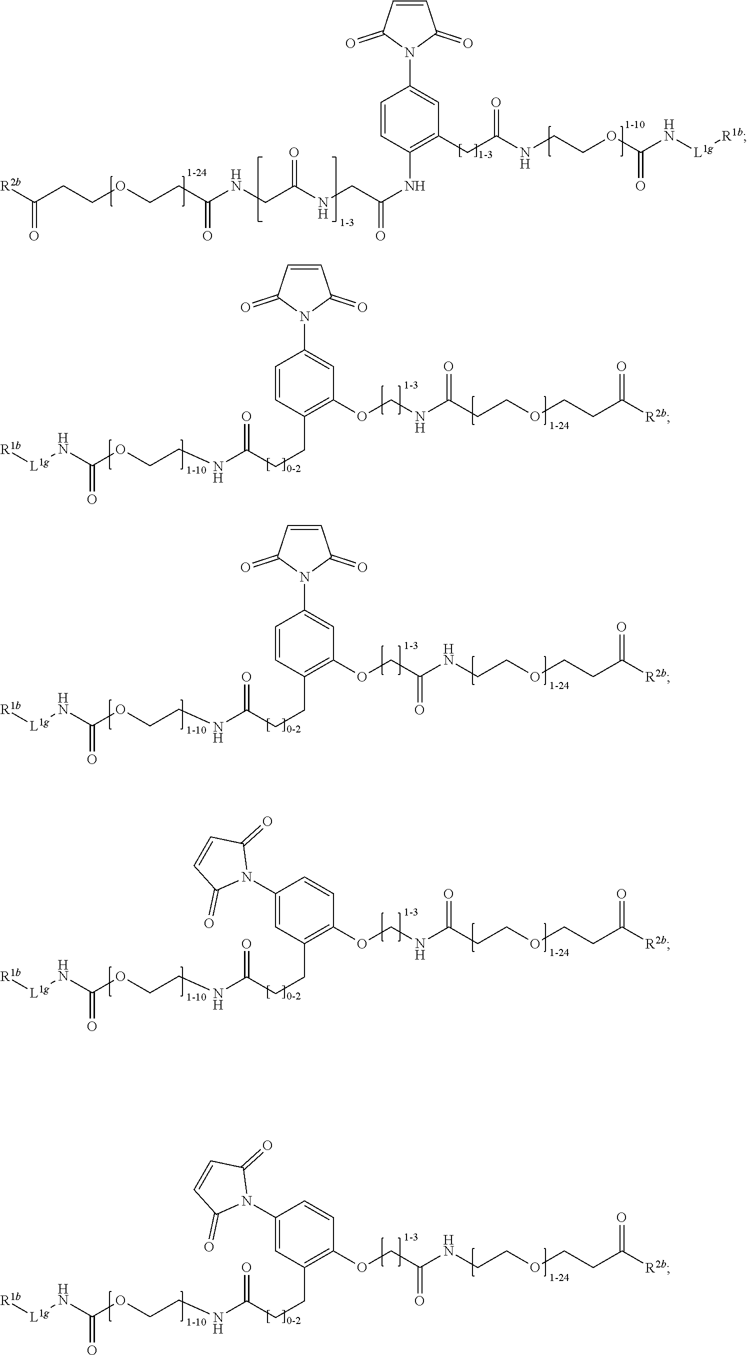

wherein:
R^{1b} is a chemotherapeutic, a cytotoxic agent, or a myeloid cell agonist;
R^{2b} has one of the following structures:

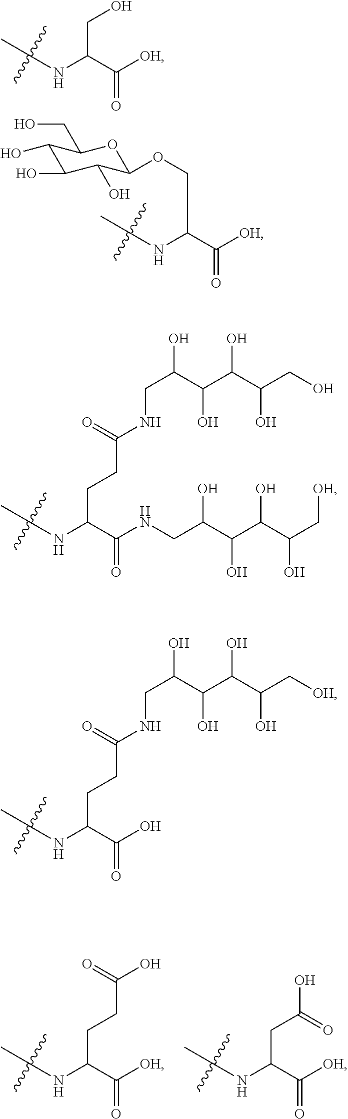

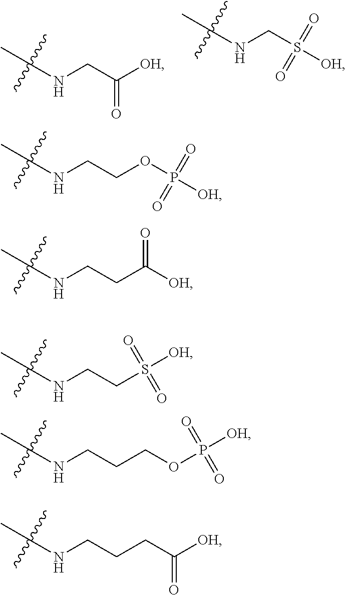

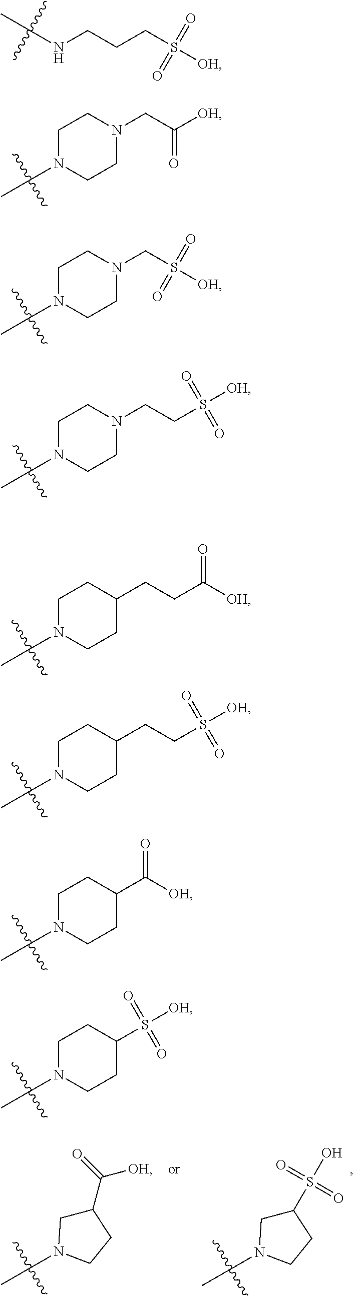

L^{1g} has one of the following structures:

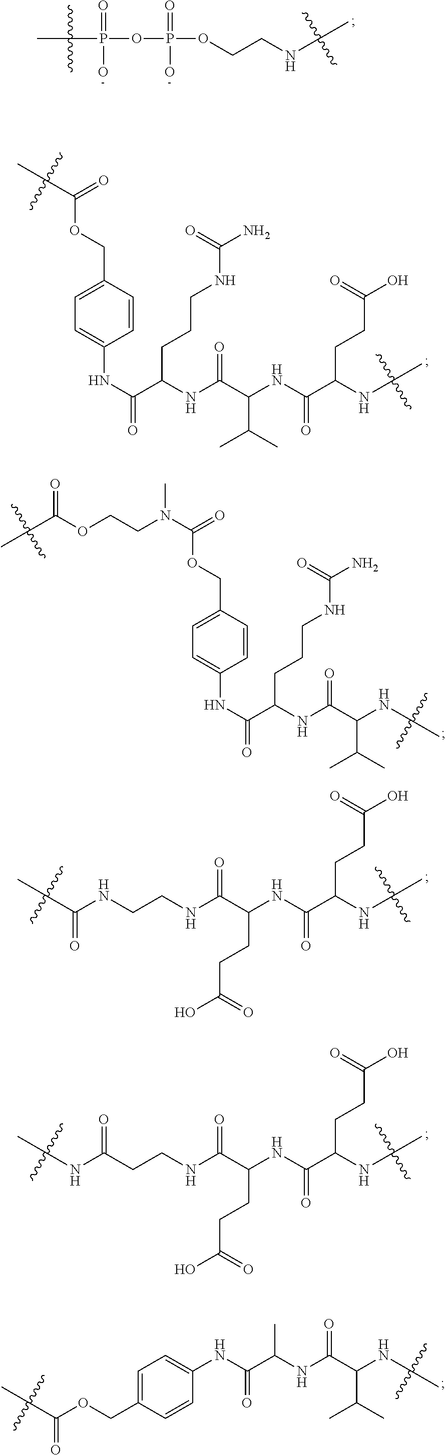

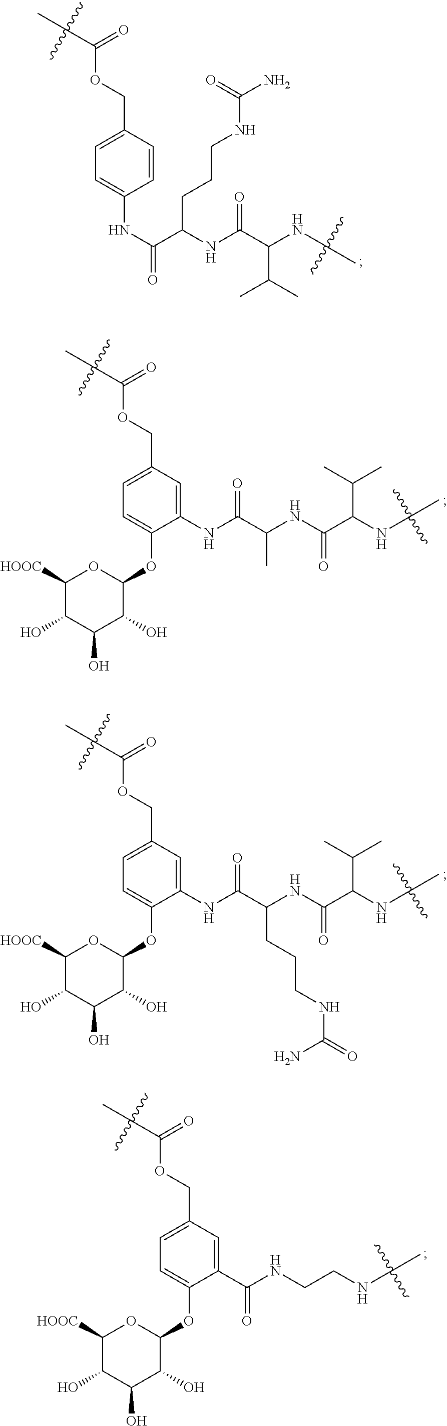 [SMILES: *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C ; *NCCNC(=O)c1cc(COC(*)=O)ccc1O[C@@H]1O[C@H](C(=O)O)[C@@H](O)[C@H](O)[C@H]1O]

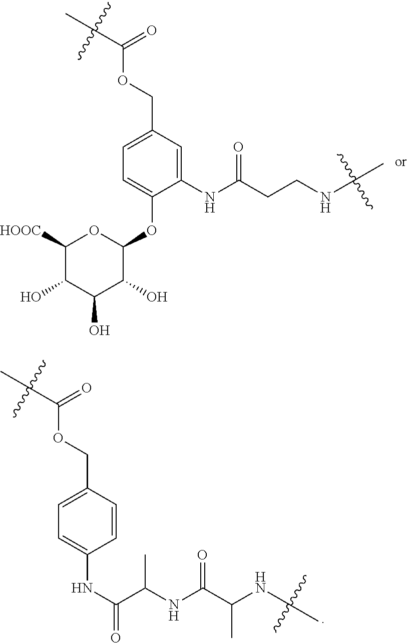 [SMILES: *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the compound has one of the following structures:

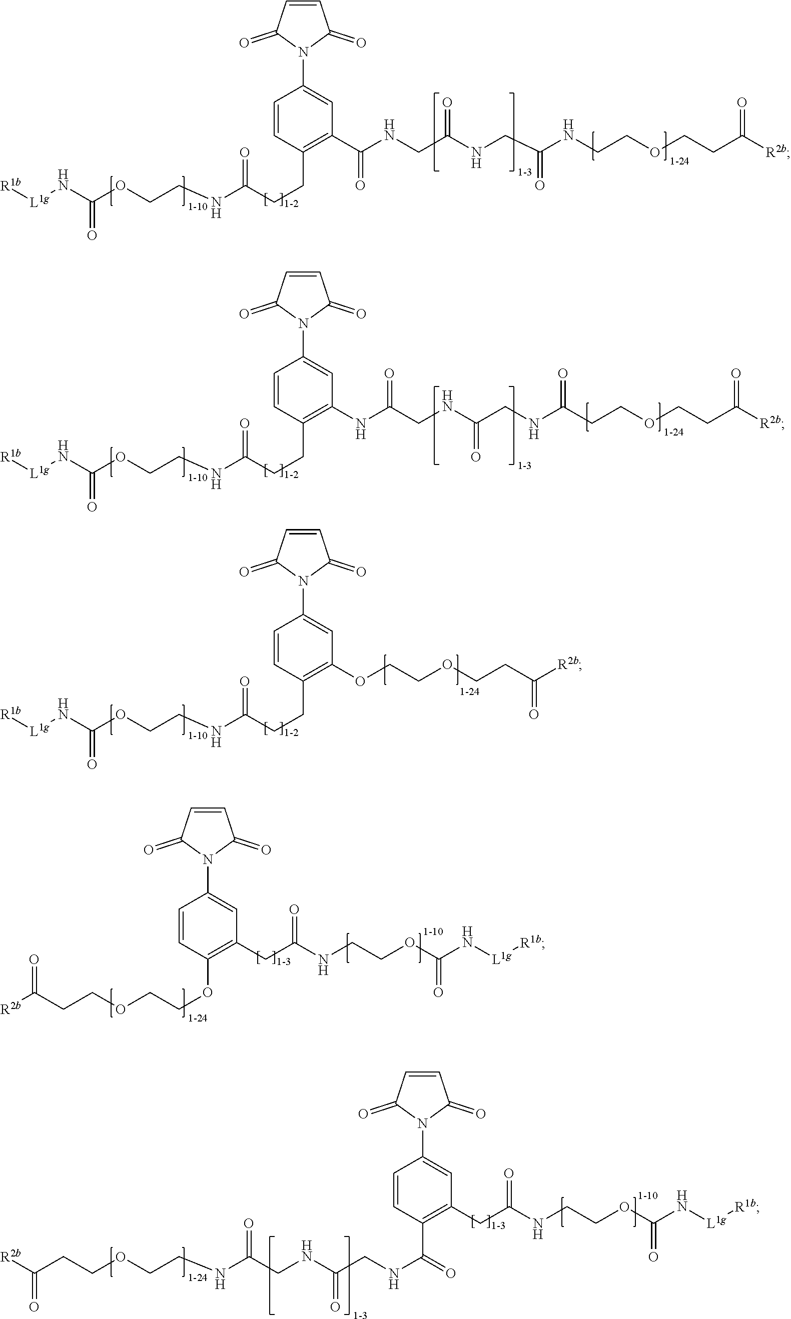

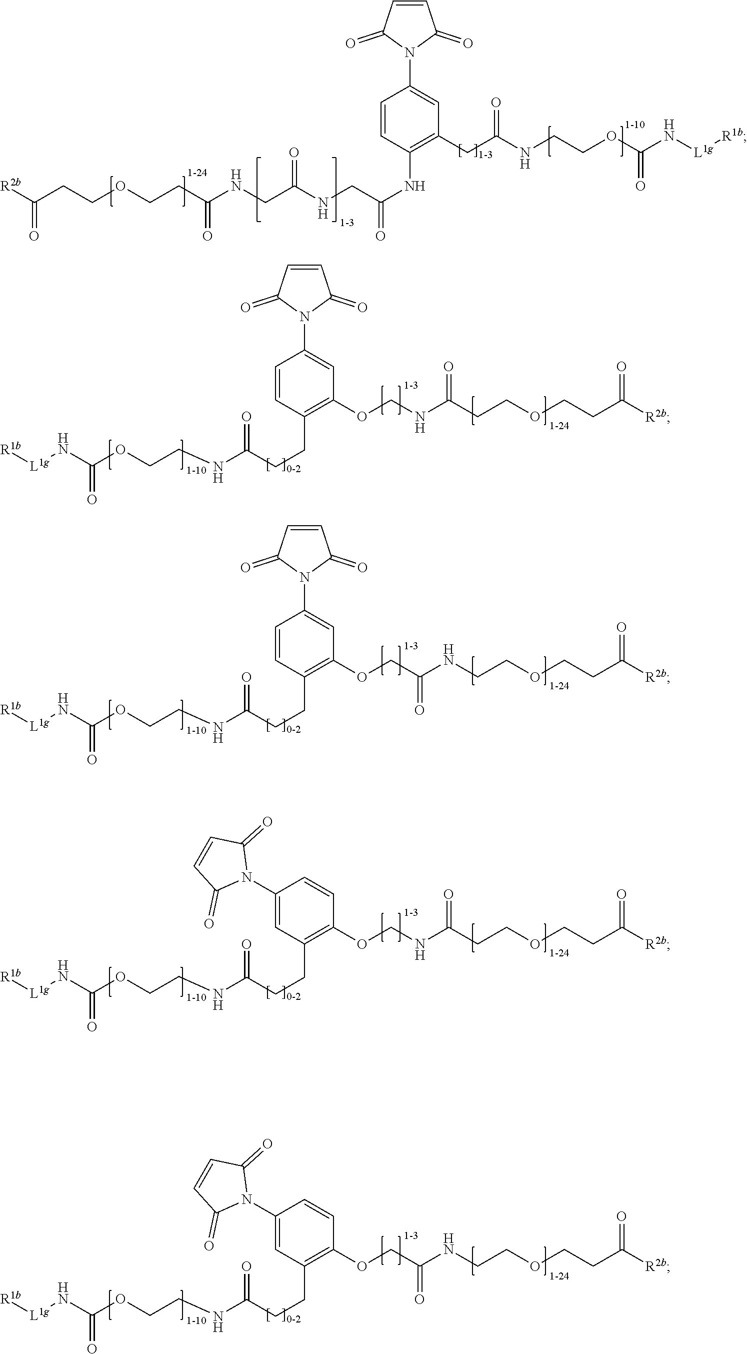

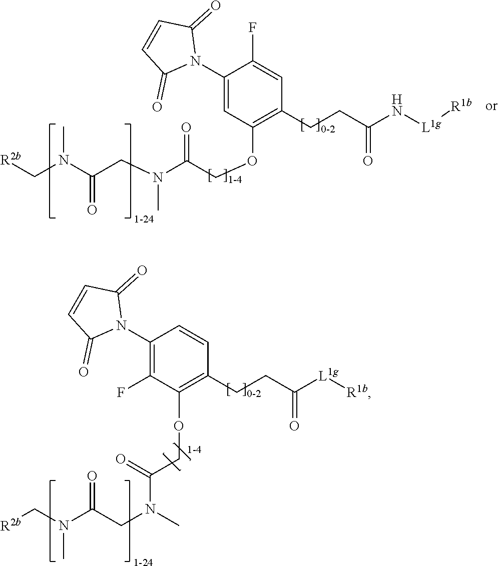

wherein
   R^{1b} is a chemotherapeutic, a cytotoxic agent, or a myeloid cell agonist;
   R^{2b} has one of the following structures:

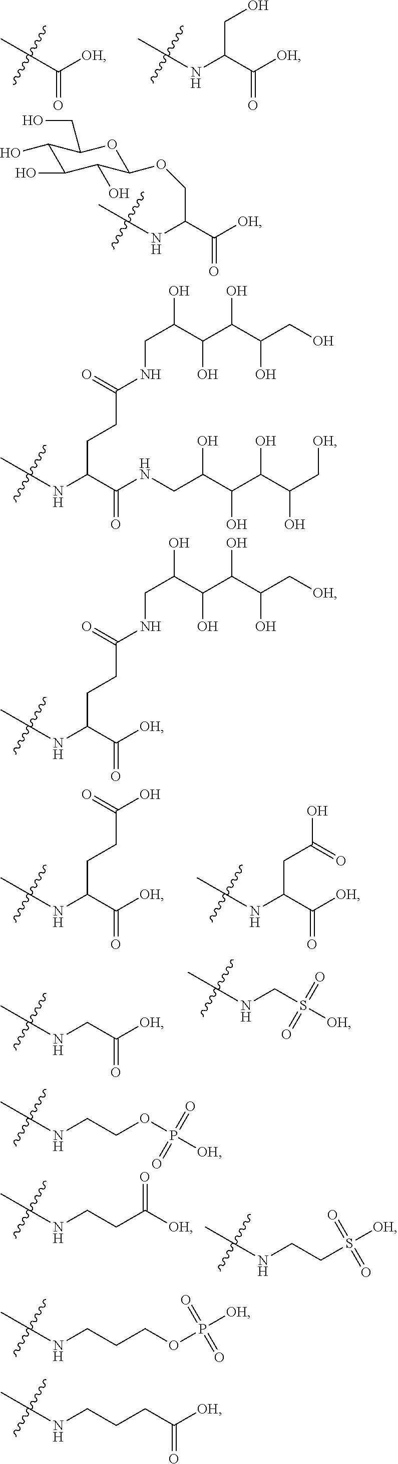

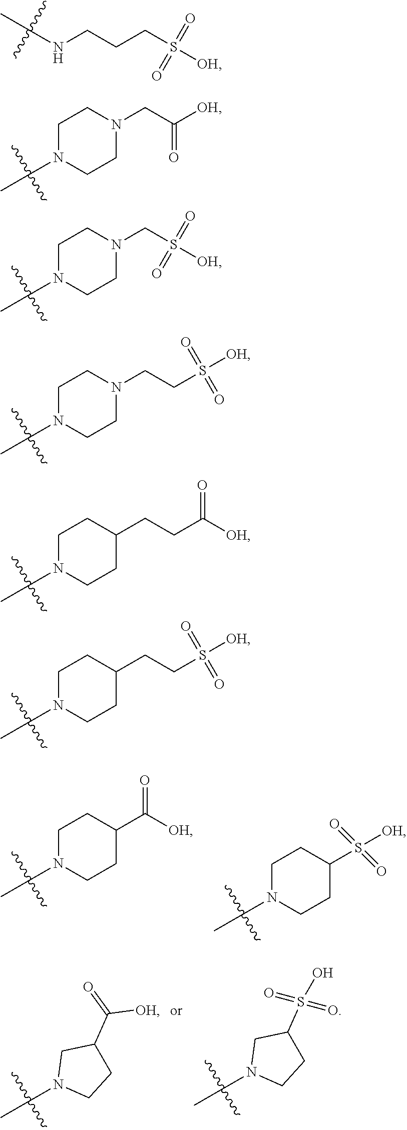

L^{1g} has one of the following structures:

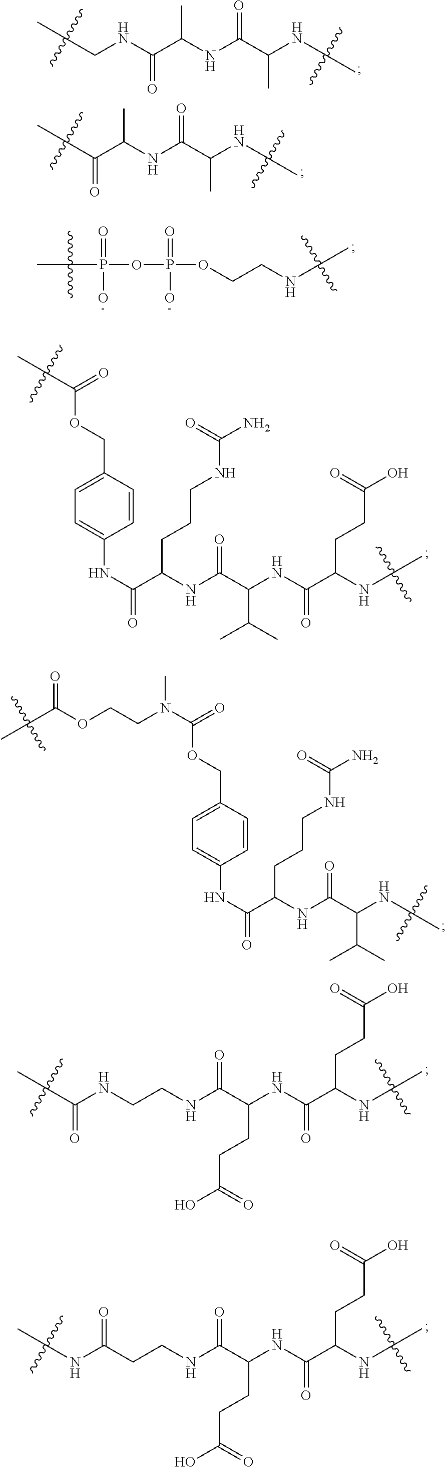

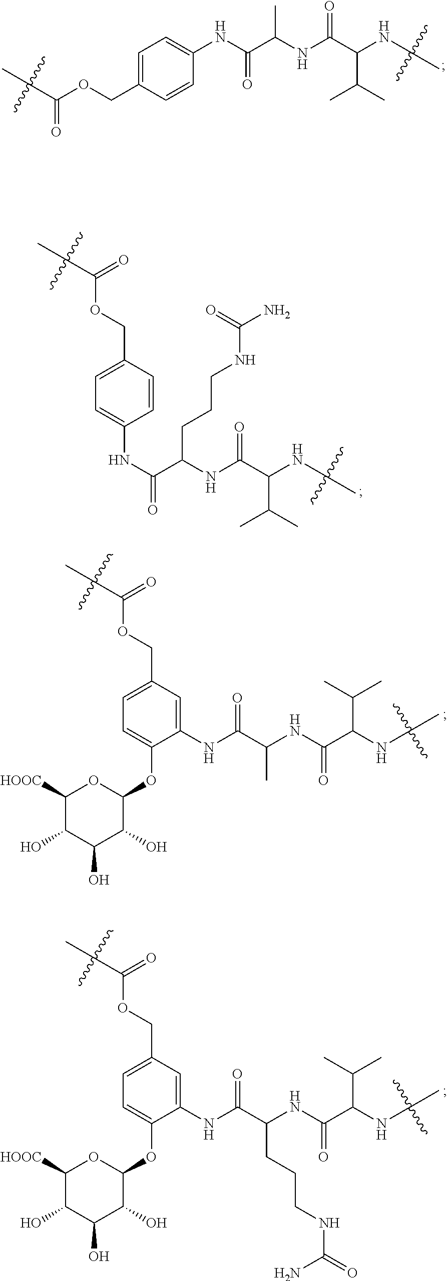 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

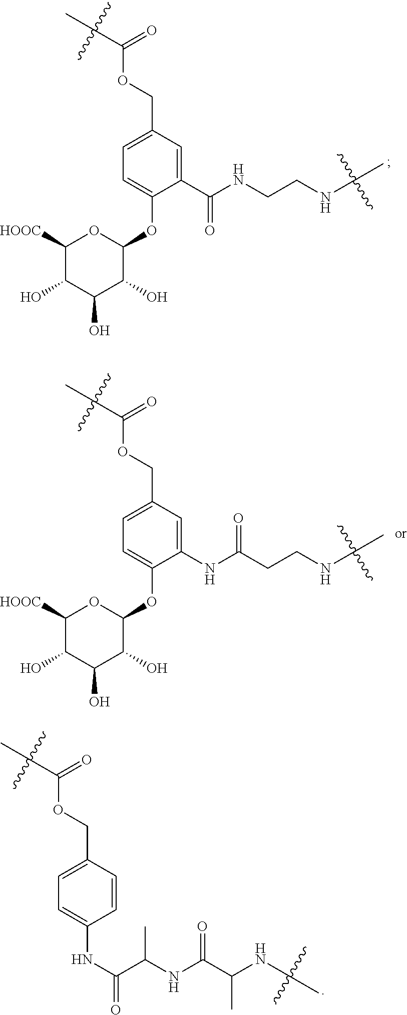 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the compound has one of the following structures:

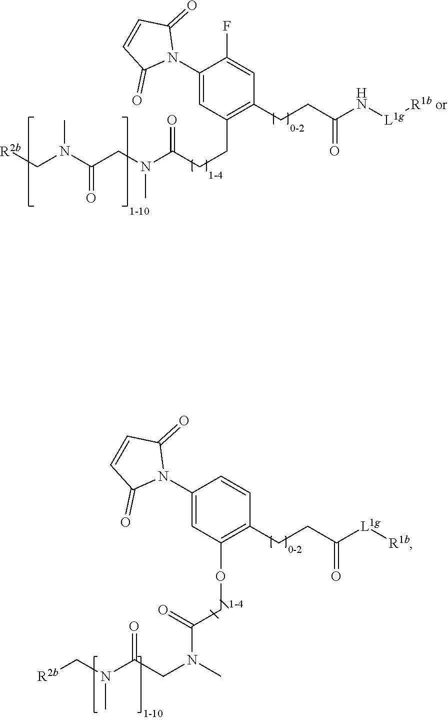

In some embodiments, the compound has one of the following structures:

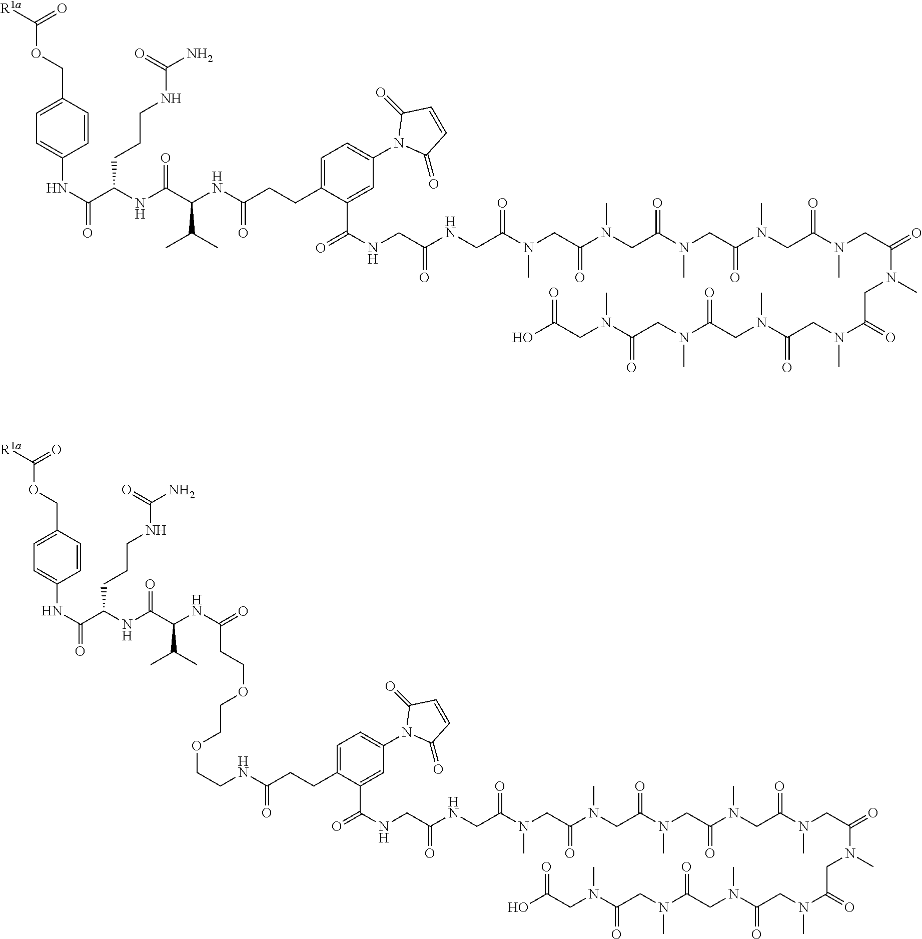

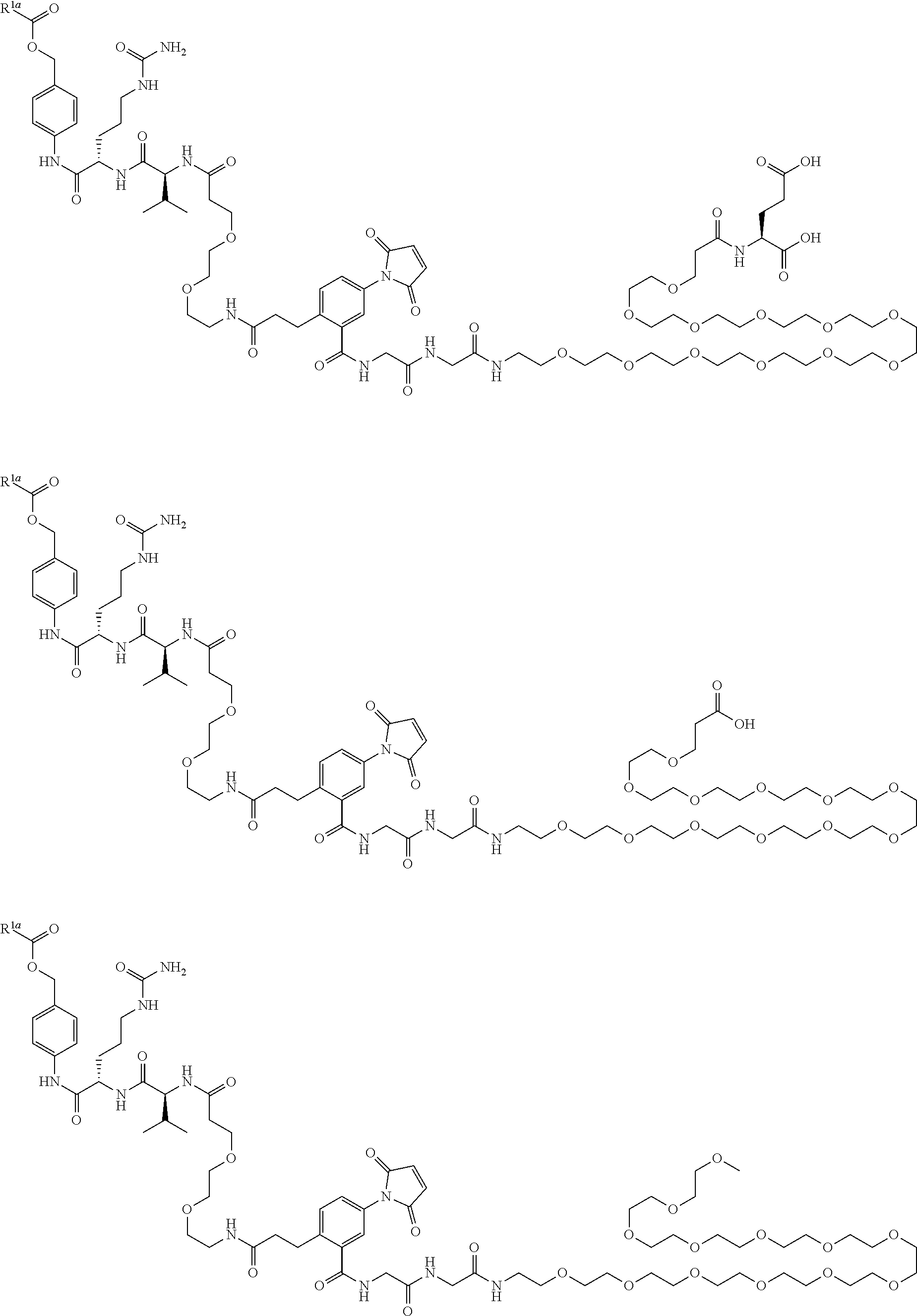

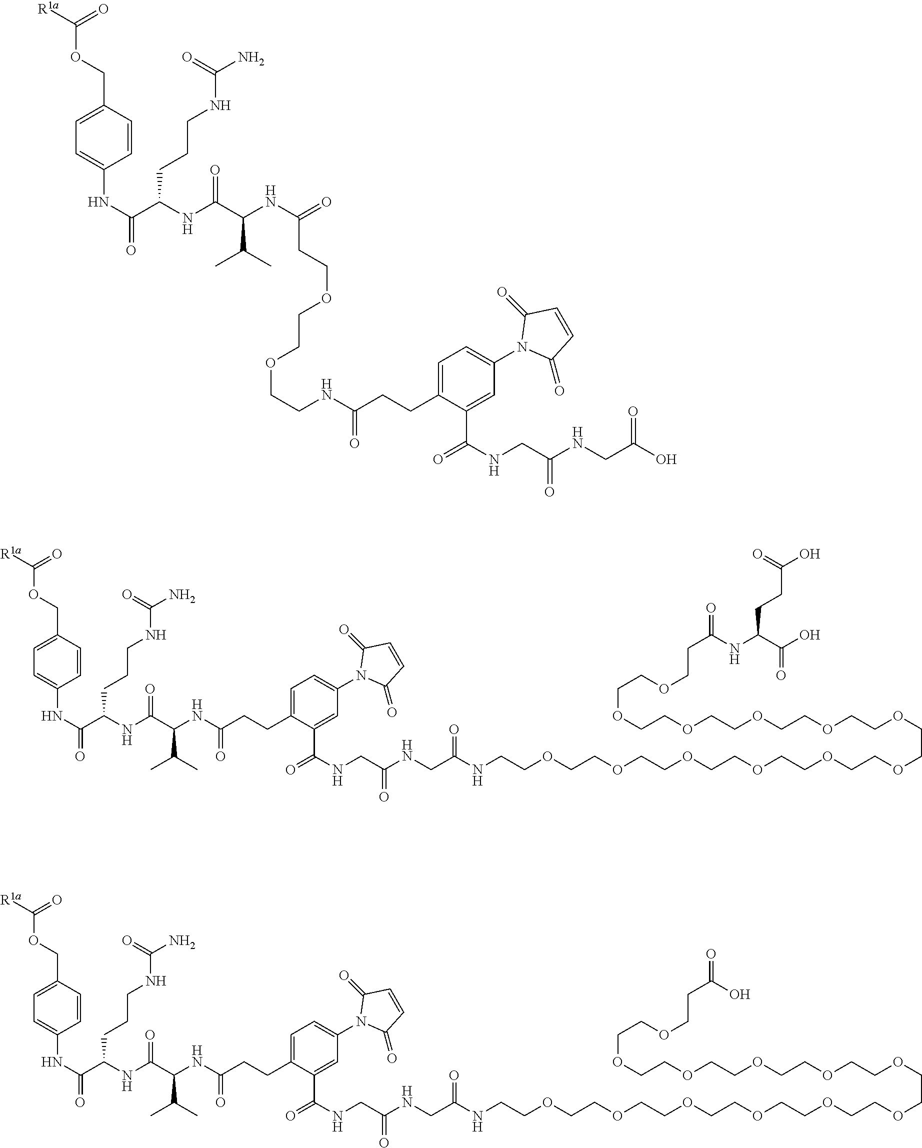

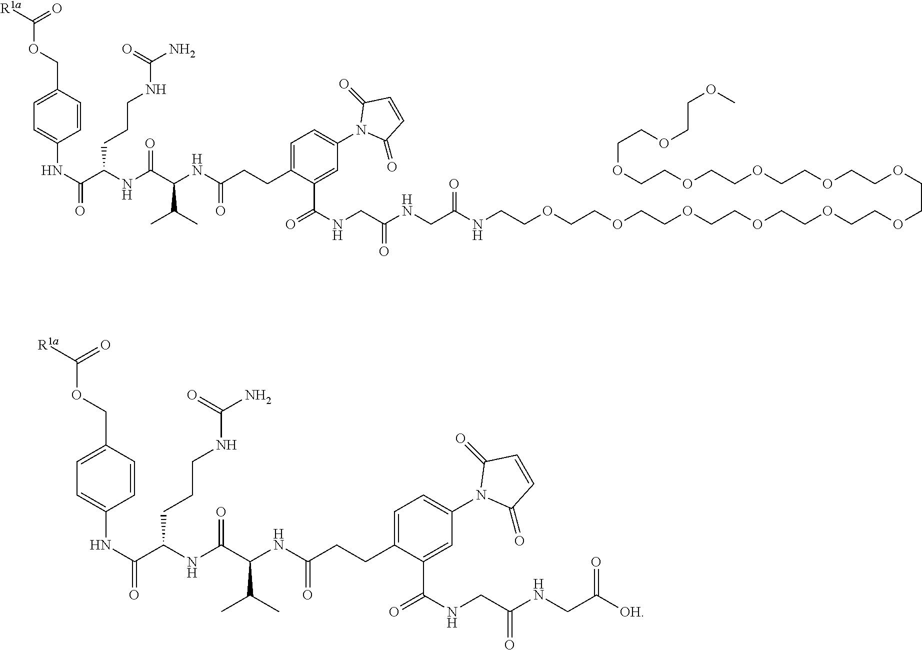

In some embodiments, the compound has one of the following structures:

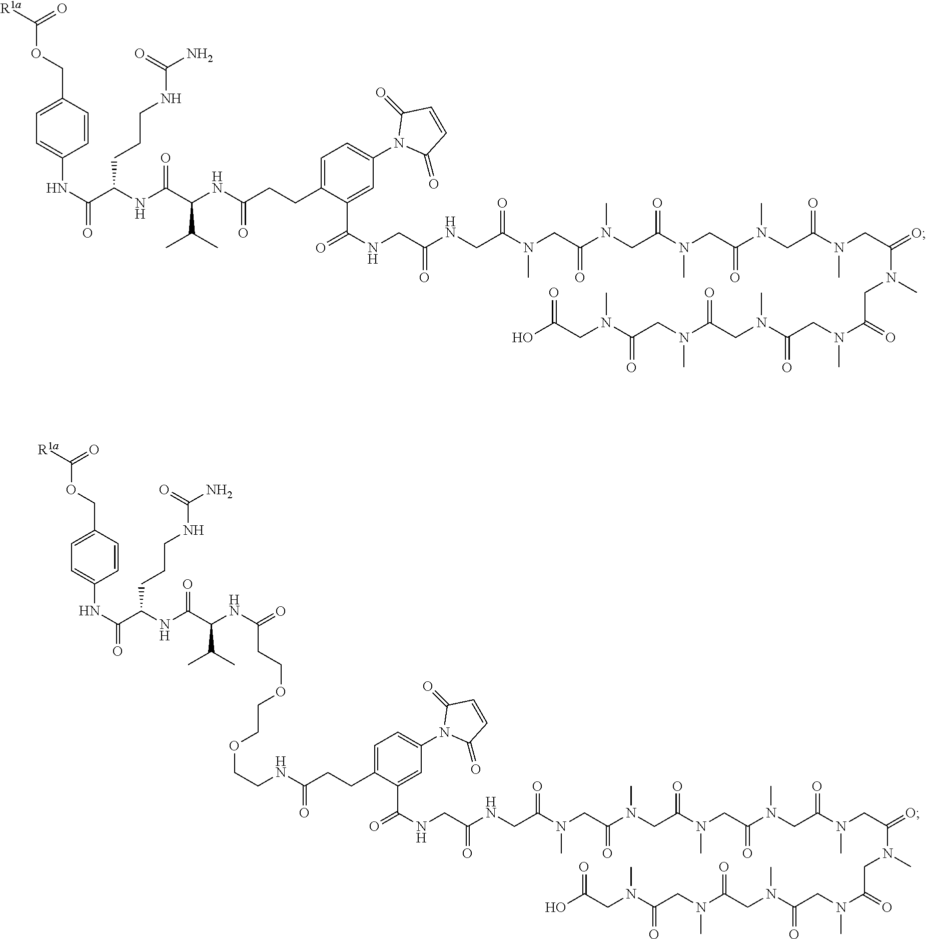

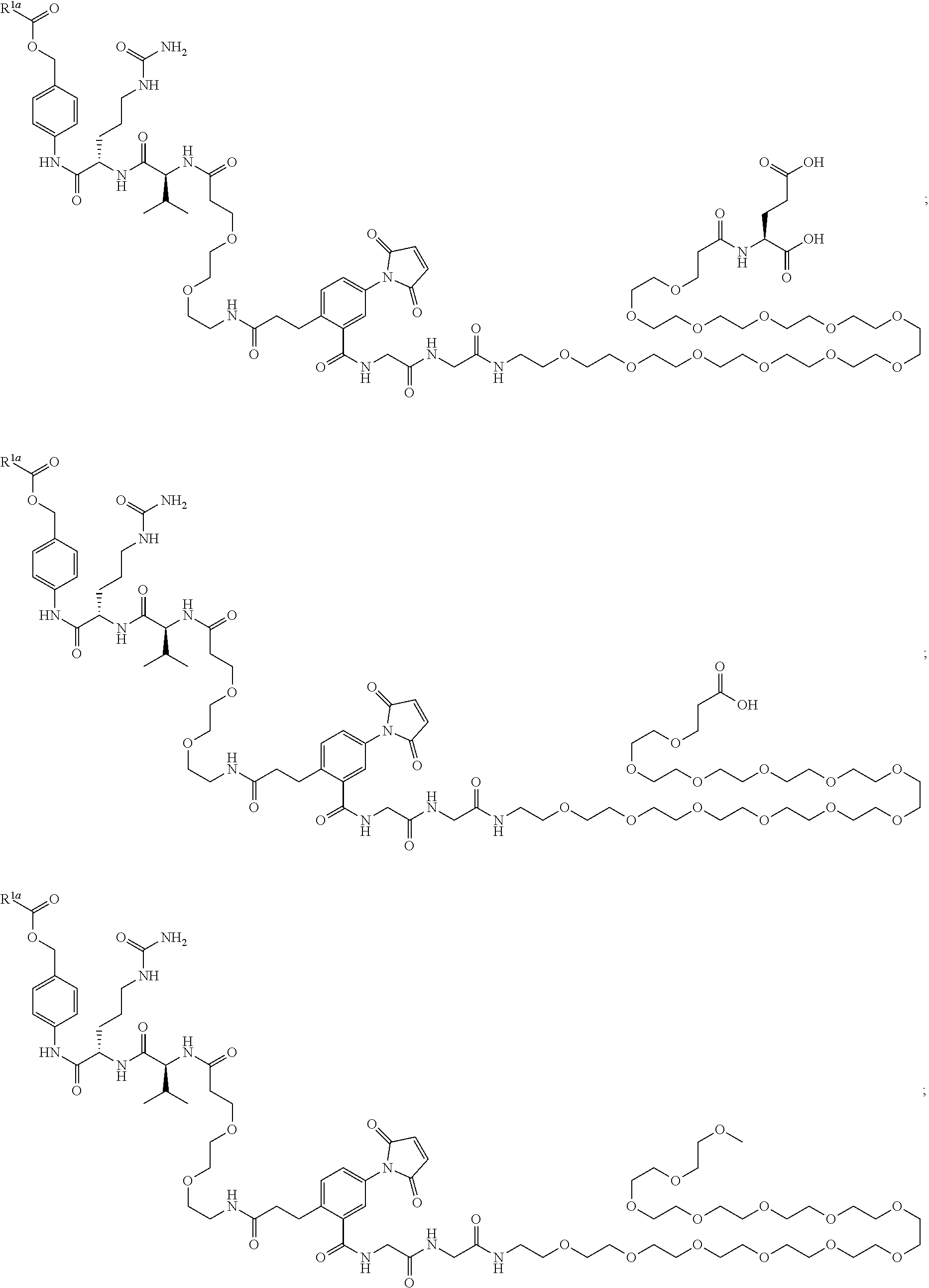

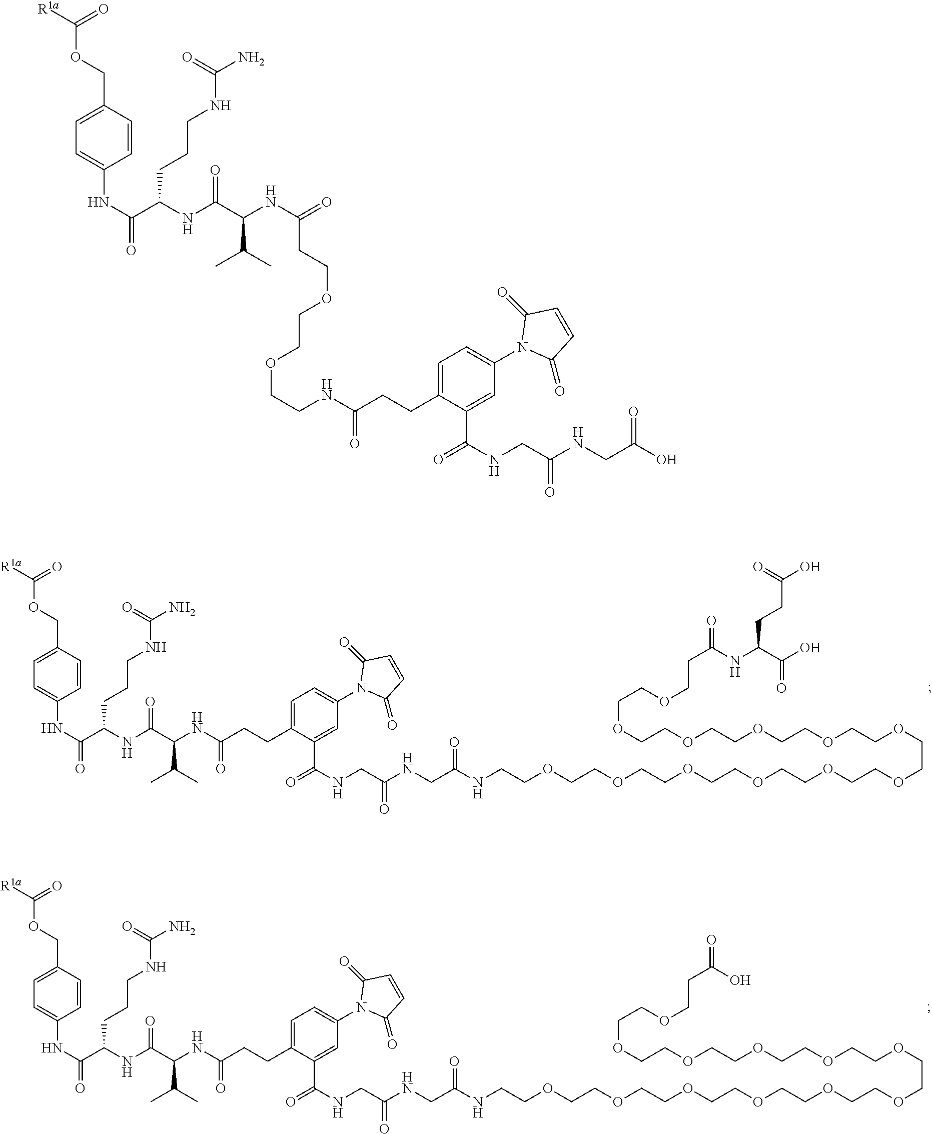

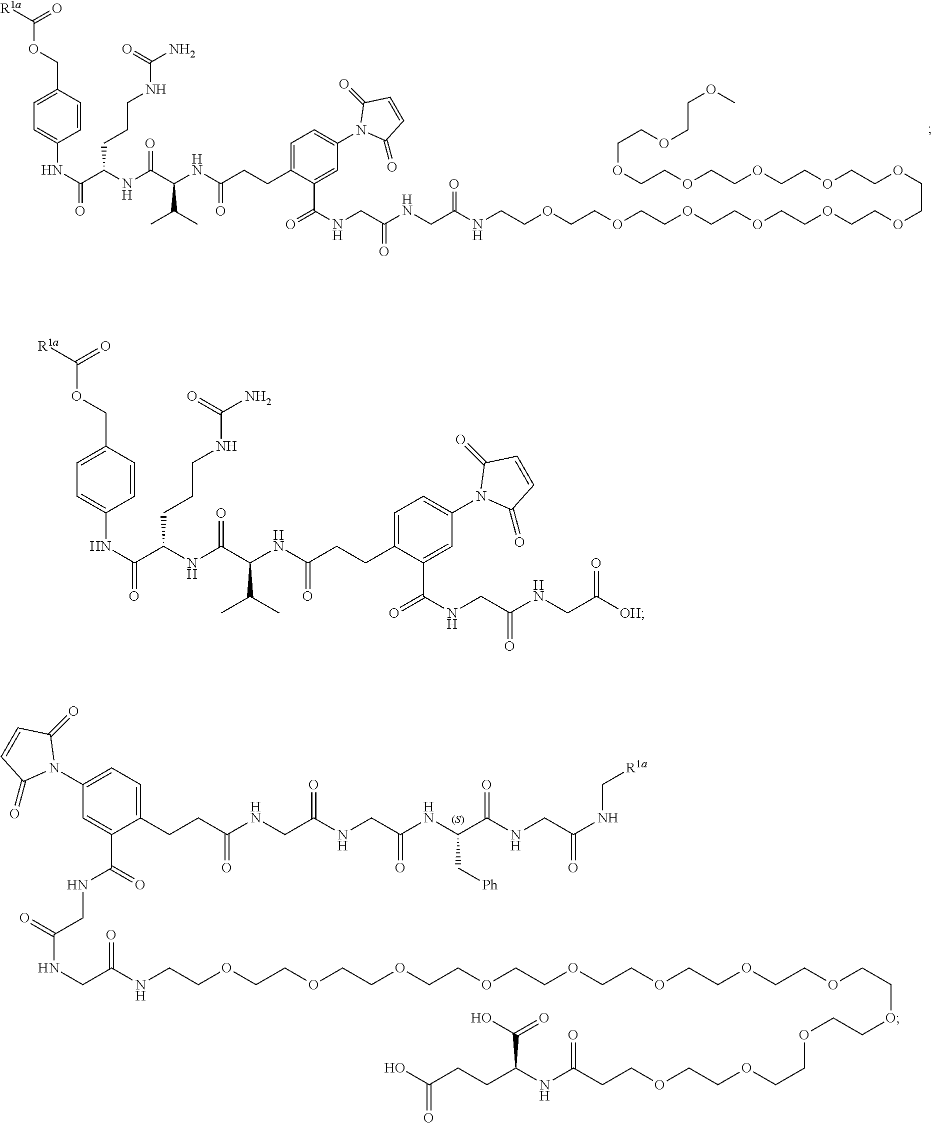

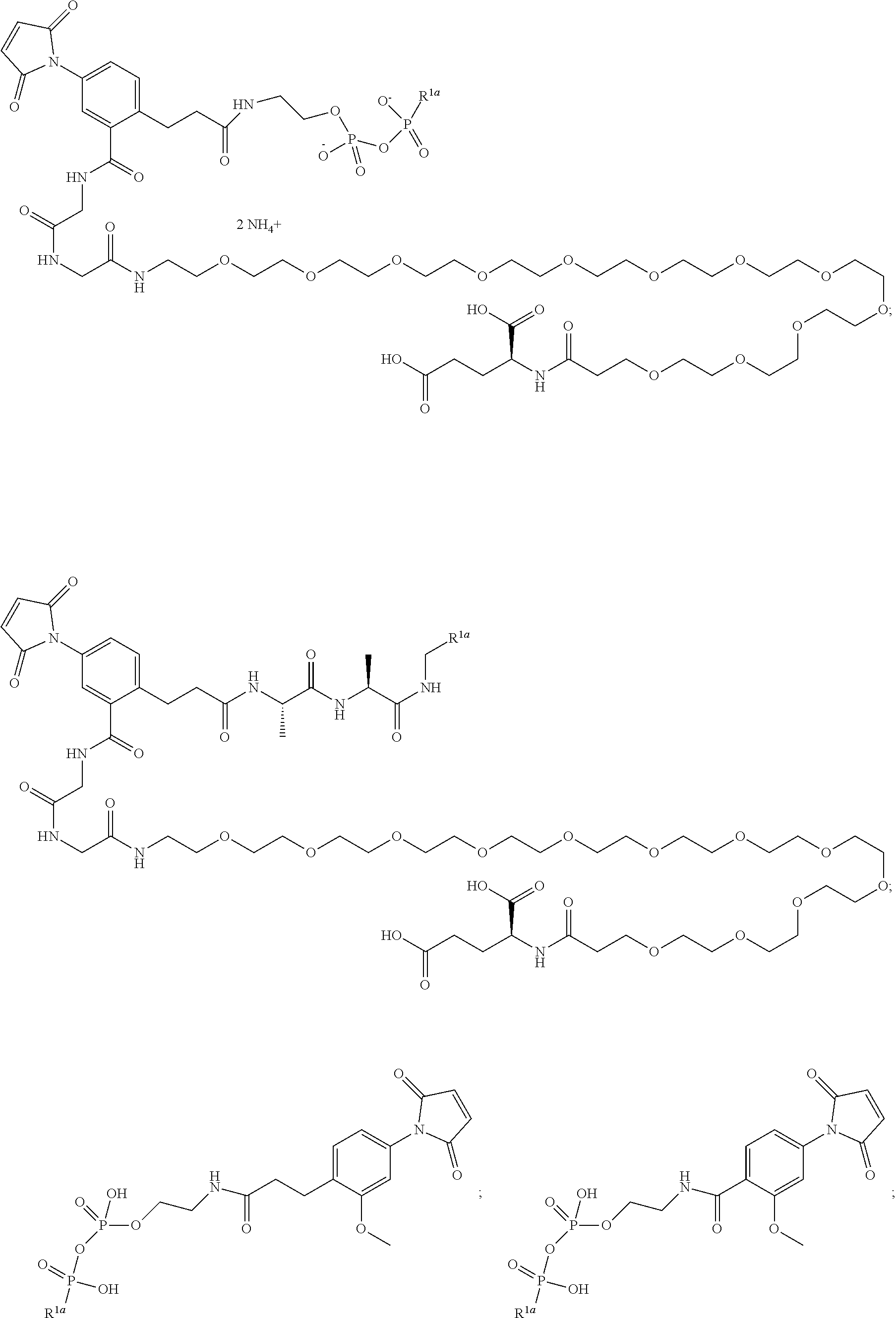

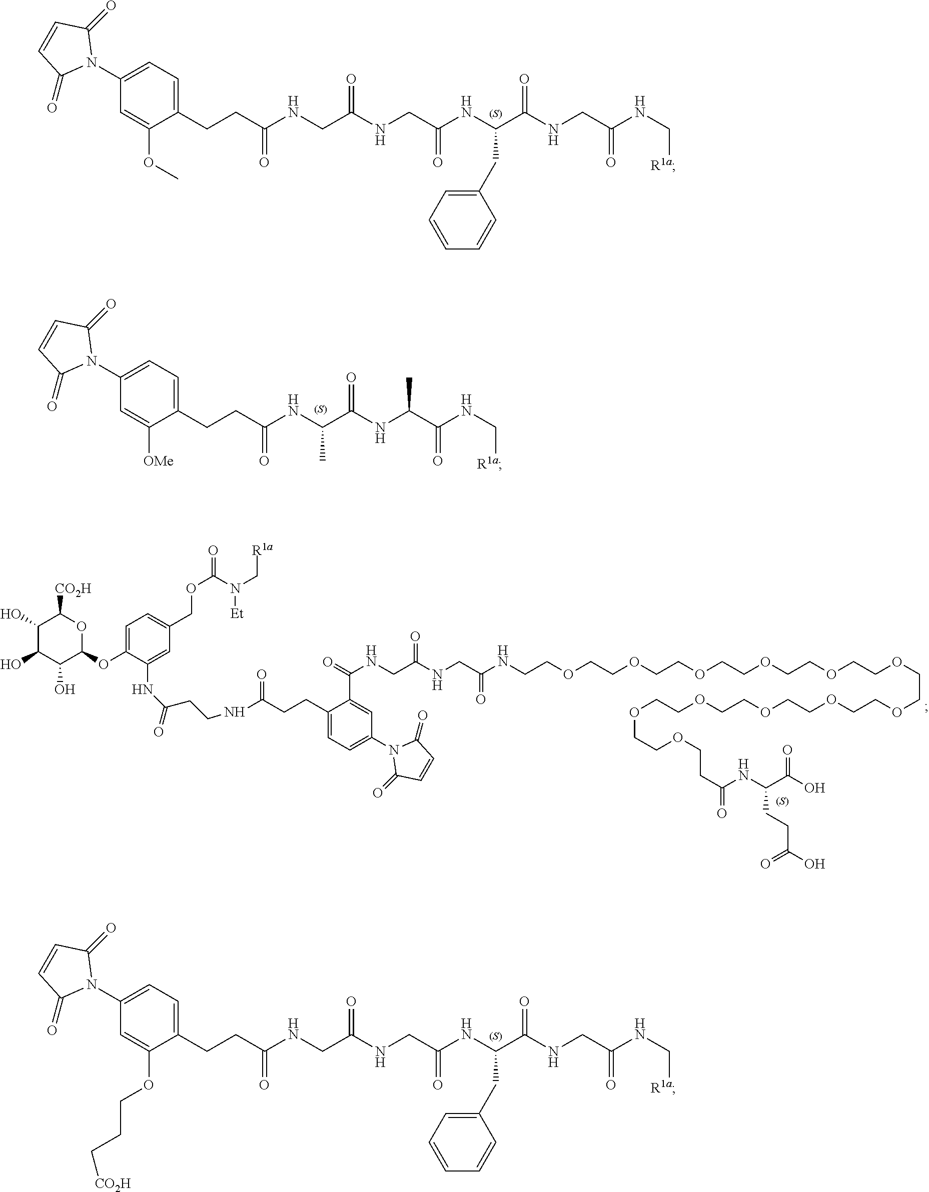

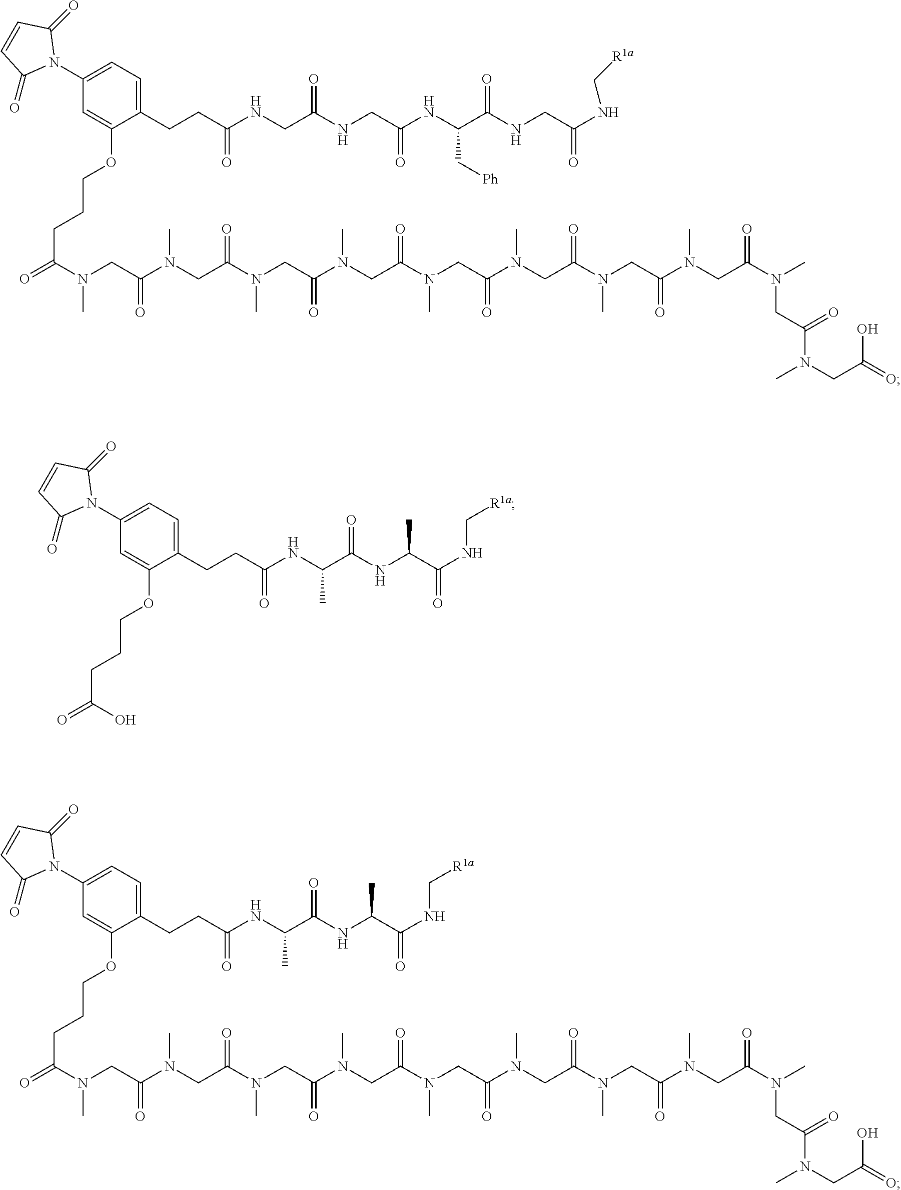

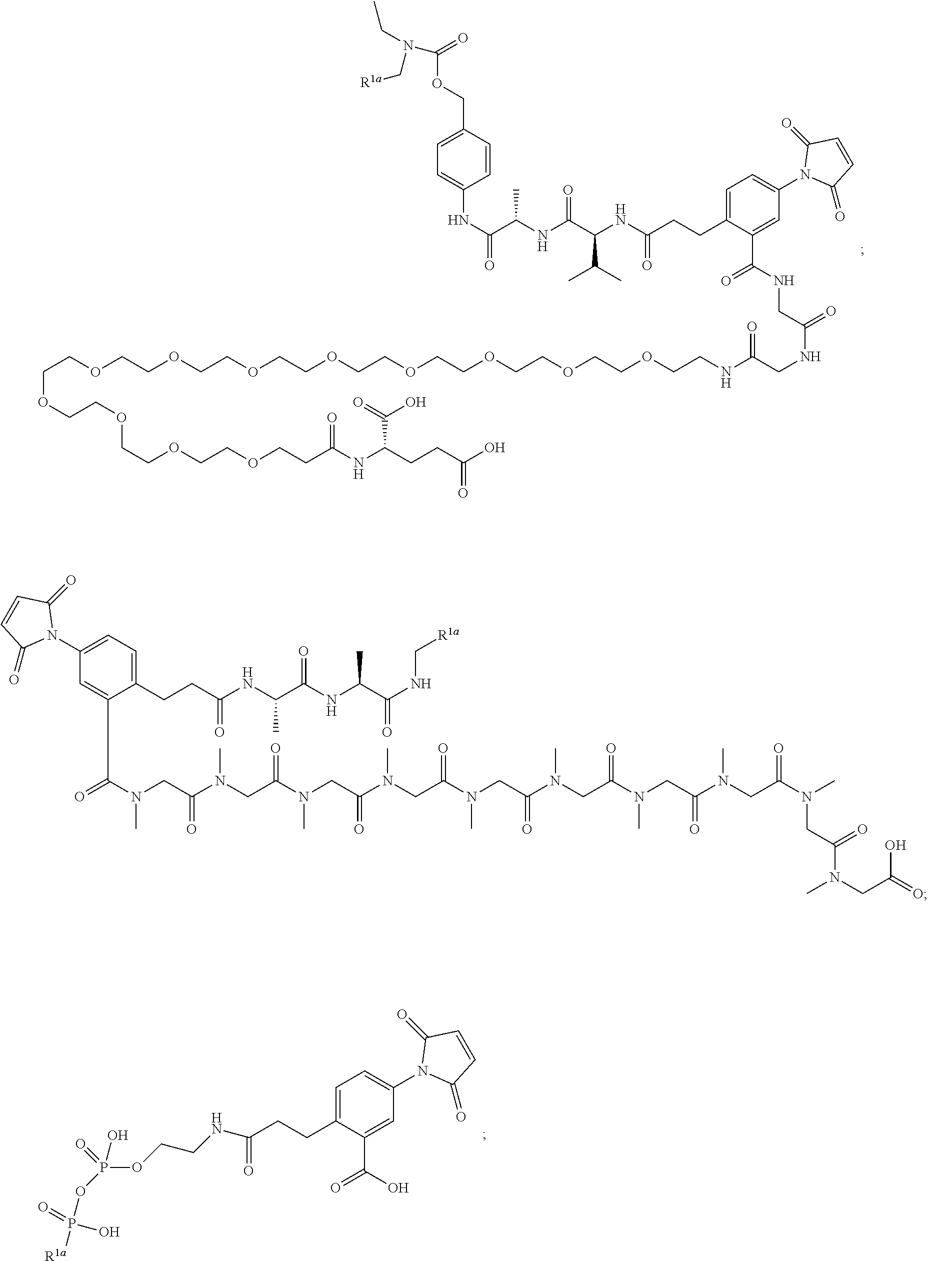

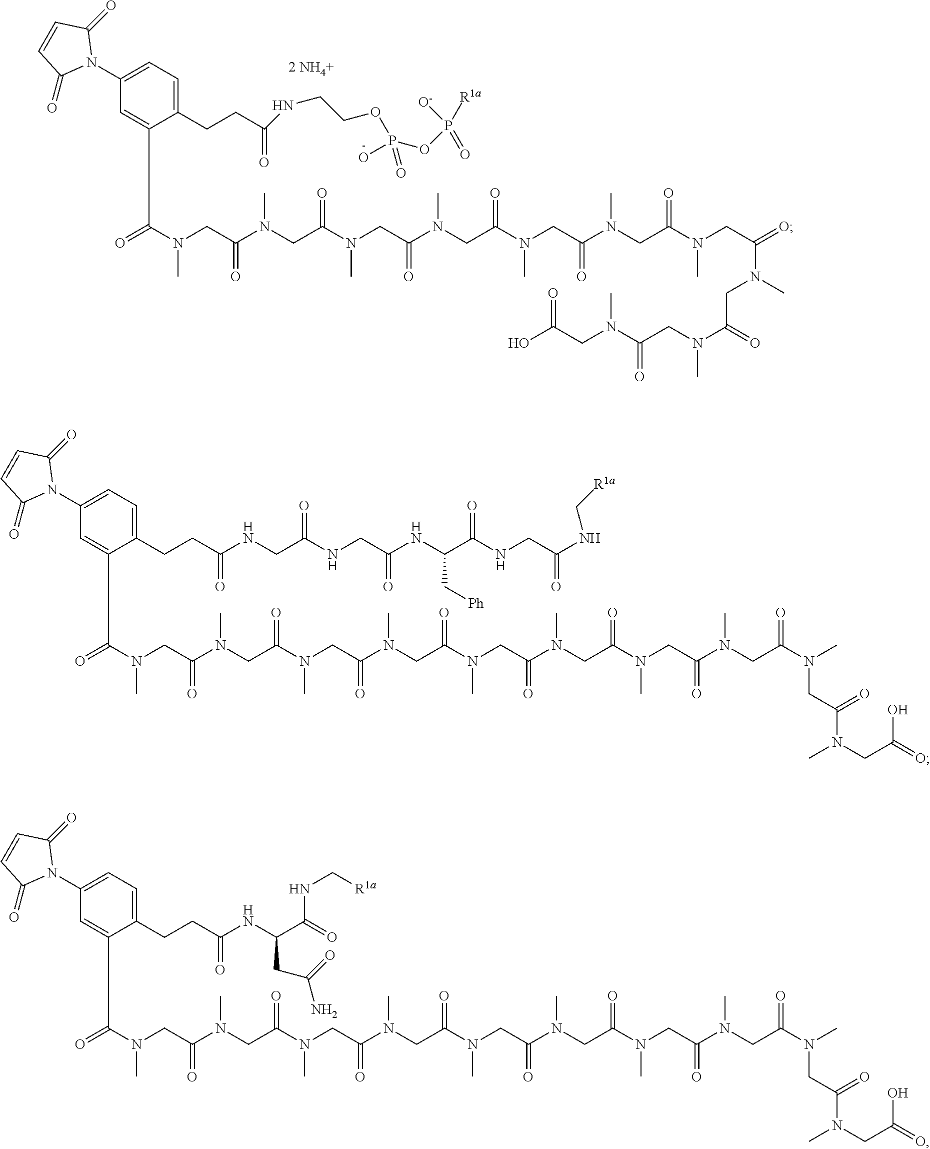

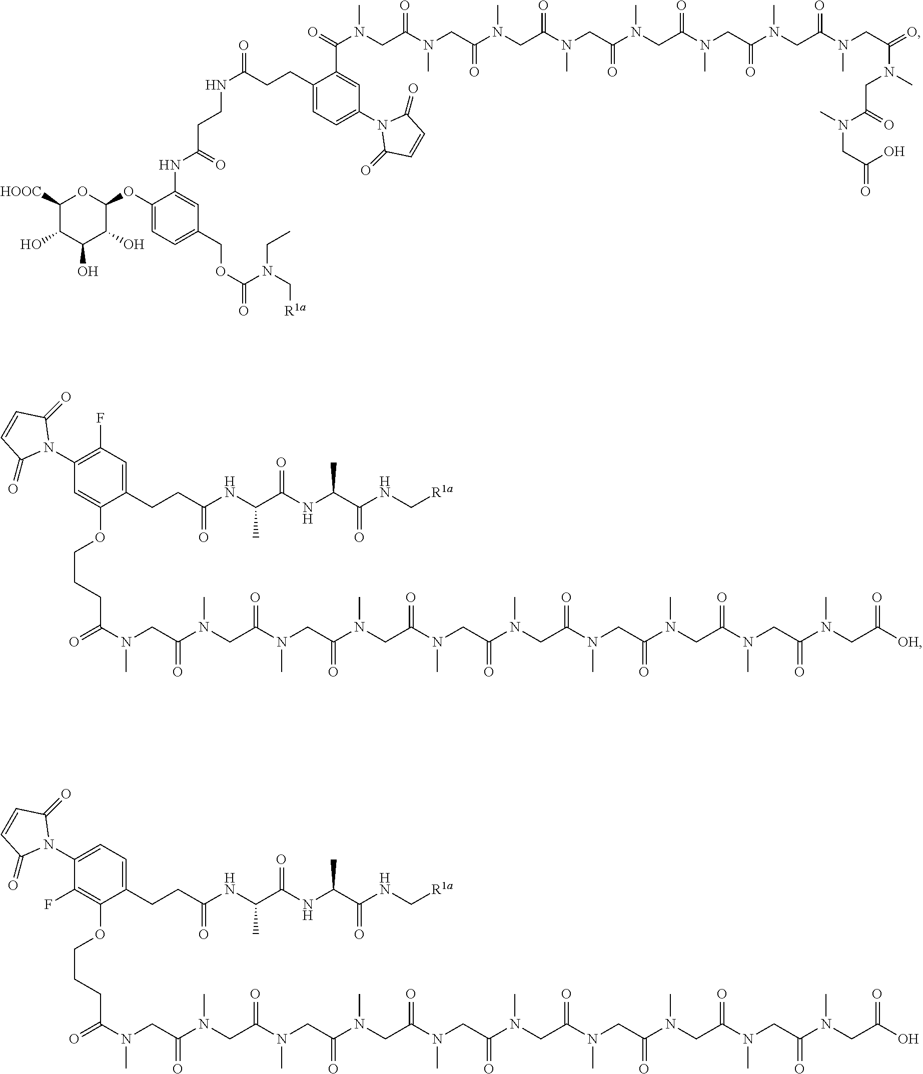

In some embodiments, a conjugate of Structure (IIx) is prepared from the linker of Structure (Ix).

Accordingly, an additional embodiment provides a conjugate having the following Structure (IIx):

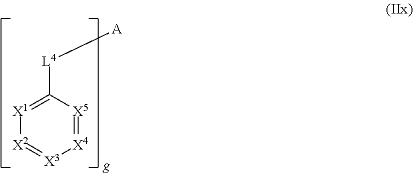

wherein:
   A is a targeting moiety or a binding fragment thereof,
   L⁴ has one of the following structures:

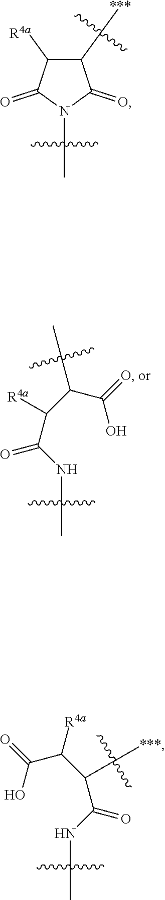

*** indicates an attachment point to A;
g is an integer from 1-20;
one of X¹, X², X³, X⁴ and X⁵ is C-L¹-R¹, another one of X¹, X², X³, X⁴ and X⁵ is C-L²-R², and the remaining three of X¹, X², X³, X⁴ and X⁵ are each independently N, C—R³, or C-L³-R^{3a};
R¹, R², and R^{3a} each independently comprise one or more moieties selected from an amino acid element, a charged element, a heteroalkylene element, a hydrophilic element, a trigger element, an immolative element, a polar cap, a compound of Structure (I) or (II), and combinations thereof;
provided that at least one of R¹ and R² comprises a compound of Structure (I) or (II);
   each occurrence of R³ is independently selected from the group consisting of hydrogen, deuterium, alkyl, haloalkyl, halo, alkoxy, haloalkoxy, amino, aminyl, amidyl, aldehyde, hydroxyl, cyano, nitro, thiol, carboxy, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide, and a carbohydrate;
   R^{4a} is hydrogen, deuterium, halo, or —S—R^{4c} wherein R^{4c} is substituted or unsubstituted C₆-C₁₀ aryl or substituted or unsubstituted 5-12 membered heteroaryl; and
   L¹, L², and L³ are each independently a linker comprising an optionally substituted alkylene, an optionally substituted alkenylene, an optionally substituted alkynylene, an optionally substituted heteroalkylene, an optionally substituted heteroalkenylene, an optionally substituted heteroalkynylene, a heteroatomic linker, an optionally substituted cycloalkylene, an optionally substituted arylene, an optionally substituted heterocyclylene, an optionally substituted heteroarylene,
   or combinations thereof, as a stereoisomer, enantiomer or tautomer thereof or a mixture thereof, or a pharmaceutically acceptable salt, solvate or prodrug thereof.

In some embodiments, R¹ has the following structure:

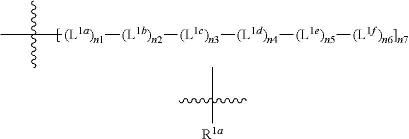

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
wherein one or more occurrence of L^{1a}L^{1b}, -L^{1c}, L^{1d}, L^{1e}, and L^{1f} optionally joins with one or more of another of L^{1a}, L^{1b}, -L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a compound of Structure (I) or (II) that is covalently bound to one occurrence of L^{1a}, L^{1b}, L^{1c}, L^{1d}, L^{1e}, or L^{1f} and the compound of Structure (I) or (II) is optionally substituted with a polar cap.

In certain embodiments, R¹ has the following structure:

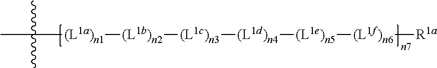

wherein:
L^{1a} is an amino acid element;
L^{1b} is a charged element;
L^{1c} is a heteroalkylene element;
L^{1d} is a hydrophilic element;
L^{1e} is a trigger element; and
L^{1f} is an immolative element;
wherein one or more occurrence of L^{1a}, L^{1b}, L^{1c}, L^{1d}, L^{1e}, and L^{1f} optionally joins with one or more of another of L^{1a}, L^{1b}, L^{1c}, L^{1d}, L^{1e}, and L^{1f} to form one or more ring;
each occurrence of n1, n2, n3, n4, n5, and n6 is independently an integer from 0-3, provided that n1+n2+n3+n4+n5+n6=1 or more;
n7 is 1, 2, 3, 4, 5, or 6; and
R^{1a} is a compound of Structure (I) or (II) optionally substituted with a polar cap.

In some embodiments, R² has the following structure:

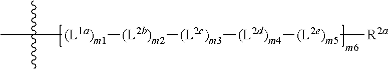

wherein:
L^{2a} is an amino acid element;
L^{2b} is a charged element;
L^{2c} is a heteroalkylene element;
L^{2d} is a hydrophilic element;
L^{2e} is a trigger element;
each occurrence of m1, m2, m3, m4, and m5 is independently an integer from 0-3, provided that m1+m2+m3+m4+m5=1 or more;
m6 is 1, 2, or 3; and
R^{2a} is hydrogen, alkyl, a compound of Structure (I) or (II), or a polar cap.

In certain embodiments, R^{3a} has the following structure:

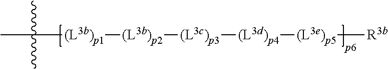

wherein:
L^{3a} is an amino acid element;
L^{3b} is a charged element;
L^{3c} is a heteroalkylene element;
L^{3d} is a hydrophilic element;
L^{3e} is a trigger element;
each occurrence of p1, p2, p3, p4, and p5 is independently an integer from 0-3, provided that p1+p2+p3+p4+p5=1 or more;
p6 is 1, 2, or 3; and
R^{3b} is hydrogen, alkyl, or a polar cap.

In some embodiments, n7 is 1 and each of n1 through n6 are 1. In certain embodiments, n7 is 1 and each of n1 through n4 are 0, n5 is 1, and n6 is 1. In some embodiments, n7 is 1 and n1 is 0, n2 is 0, n3 is 1, n4 is 0, n5 is 1, and n6 is 1.

In some embodiments, m6 is 1 and each of m1 through m5 are 1. In certain embodiments, m6 is 1, m1 is 1, m2 is 0, m3 is 0, m4 is 1, and m5 is 0.

In certain embodiments, p6 is 1 and each of p1 through p5 is 1. In some embodiments, p6 is 1, p1 is 1, p2 is 0, p3 is 0, p4 is 1, and p5 is 0.

In some embodiments, an amino acid element comprises one or more amino acids selected from the group consisting of glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, arginine, sarconsine, and beta-alanine. In more embodiments, an amino acid element comprises one or more amino acids selected from the group consisting of glycine, sarcosine, beta-alanine, and glutamic acid.

In some embodiments, an amino acid element comprises a dipeptide, a tripeptide, a tetrapeptide, or a pentapeptide. In some embodiments, an amino acid element has one of the following structures:

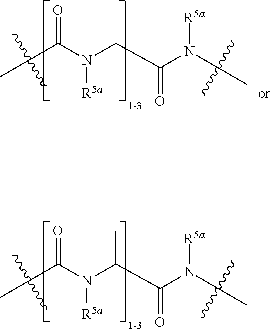

wherein:
each occurrence of R^{5a} is independently hydrogen, alkyl, hydroxyalkyl, or alkoxyalkyl.

In some embodiments, a charged element comprises moieties with a negative charge at pH 7.4 (i.e., a range from 6.3 to 8.5). In some embodiments, a charged element comprises moieties with a positive charge at pH 7.4 (i.e., a range from 6.3 to 8.5).

In some embodiments, a charged element comprises one or more charged amino acid, one or more carboxylic acid, one or more sulfonic acid, one or more sulfonamide, one or more sulfate, one or more phosphate, one or more quaternary amine, one or more sulfamide, one or more sulfinimide, or combinations thereof.

In some embodiments, a charged amino acid is aspartic acid, glutamic acid, histidine, lysine, or arginine.

In some embodiments, a heteroalkylene element comprises polyethylene glycol or polypropylene glycol. In some embodiments, a heteroalkylene element comprises one of the following structures:

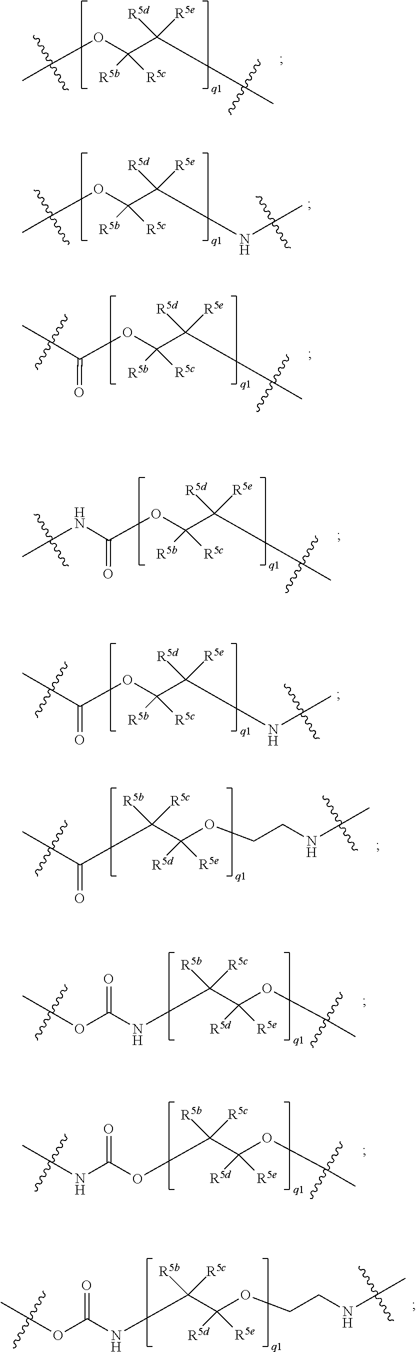

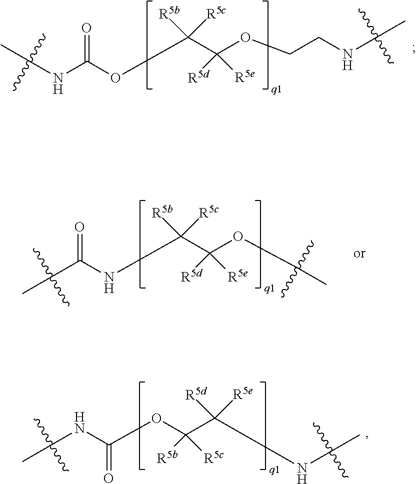

wherein:
   each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
each occurrence of q1 is, independently an integer from 1-24.

In some embodiments, q1 is 8, 10, 12, or 14.

In some embodiments, R¹, R², or R^{3a} comprises a non-cleavable linker.

In more embodiments, R¹, R², or R^{3a} comprises one of the following structures:

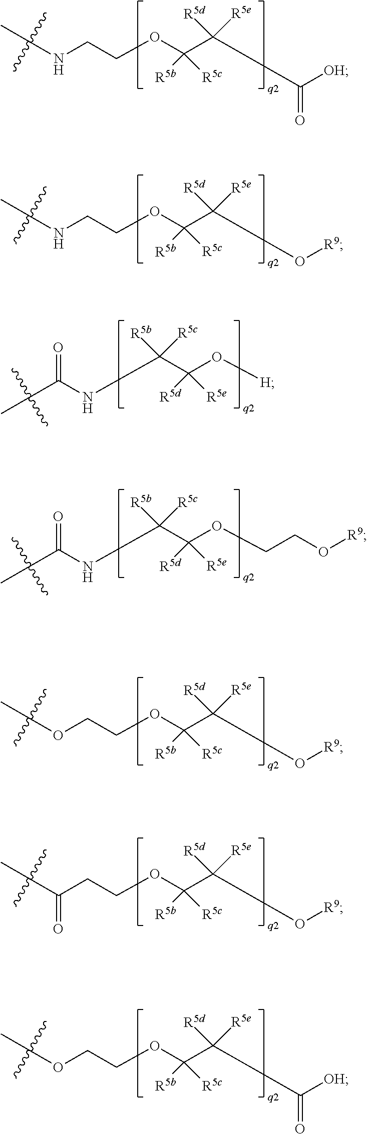

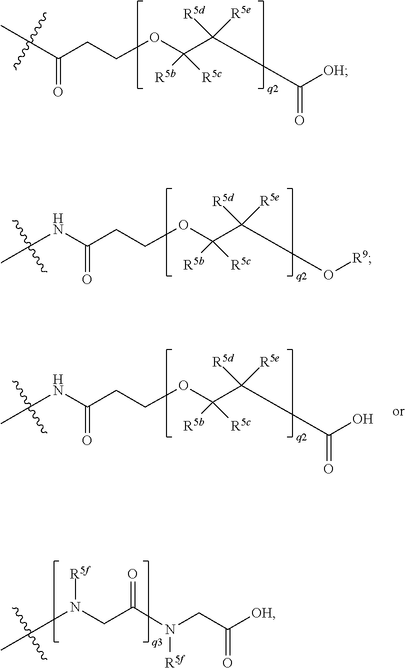

wherein:
each occurrence of R^{5b}, R^{5c}, R^{5d}, and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H, alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, —P(O)₃H, alkyl-O—P(O)₃-alkyl, alkyl-P(O)₃-alkyl, —O-alkyl-S(O)₃-alkyl, —O-alkyl-O—P(O)₃-alkyl, —O-alkyl-P(O)₃-alkyl, —S(O)₃-alkyl, —OP(O)₃-alkyl, —P(O)₃-alkyl, sulfamide, sulfinimide;
each occurrence of R^{5f} is independently hydrogen, alkyl, hydroxyalkyl, or alkoxyalkyl;
each occurrence of R⁹ is independently hydrogen or alkyl;
each occurrence of q2 is independently an integer from 1-25; and
each occurrence of q3 is independently an integer from 5-15.

In some embodiments, q2 is 8, 10, 12, or 14. In some embodiments, q3 is 6, 8, 10, 12, or 14.

In some embodiments, a hydrophilic element comprises polyethylene glycol, polysarcosine, cyclodextrin, c-glycosides, or combinations thereof. In some embodiments, a hydrophilic element comprises polyethylene glycol. In some embodiments, a hydrophilic element comprises polysarcosine. In some embodiments, a hydrophilic element comprises cyclodextrin. In some embodiments, a hydrophilic element comprises c-glycosides. In some embodiments, a hydrophilic element comprises one of the following structures:

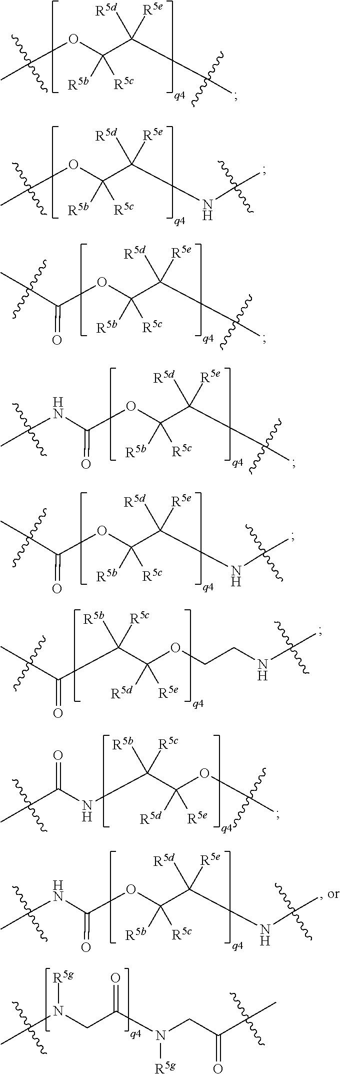

wherein:
   each occurrence of R^{5b}, R^{5c}, R^{5d} and R^{5e} is independently selected from the group consisting of hydrogen, deuterium, alkyl, haloalkyl, halo, alkoxy, haloalkoxy, amino, hydroxyl, cyano, nitro, thiol, carboxyalkyl, alkyl-S(O)₃H, alkyl-O—P(O)₃H,
   alkyl-P(O)₃H, —O-carboxyalkyl, —O-alkyl-S(O)₃H, —O-alkyl-O—P(O)₃H, —O-alkyl-P(O)₃H, —S(O)₃H, —OP(O)₃H, and —P(O)₃H,
   each occurrence of R⁵ is independently hydrogen, alkyl, hydroxyalkyl, or alkoxyalkyl; and
   each occurrence of q4 is, independently an integer from 1-24.

In some embodiments, R^{5g} is —CH₃ at each occurrence.

In some embodiments, a trigger element comprises a dipeptide, a tripeptide, a tetrapeptide, a pentapeptide, a glucuronide, a disulfide, a phosphate, a diphosphate, a triphosphate, a hydrazone, or combinations thereof. In some embodiments, a trigger element comprises beta-glucuronic acid. In certain embodiments, a trigger element comprises a dipeptide, a tripeptide, a tetrapeptide, or a pentapeptide. In certain embodiments, a trigger element comprises two or more amino acids selected from the group consisting of valine, citrulline, alanine, glycine, phenylalanine, lysine, or combinations thereof. In some embodiments, a trigger element comprises a sequence of amino acids selected from the group consisting of valine-citrulline, valine-alanine, glycine-glycine-phenylalanine-glycine, and combinations thereof. In certain embodiments, a trigger element comprises one of the following structures, including combinations thereof:

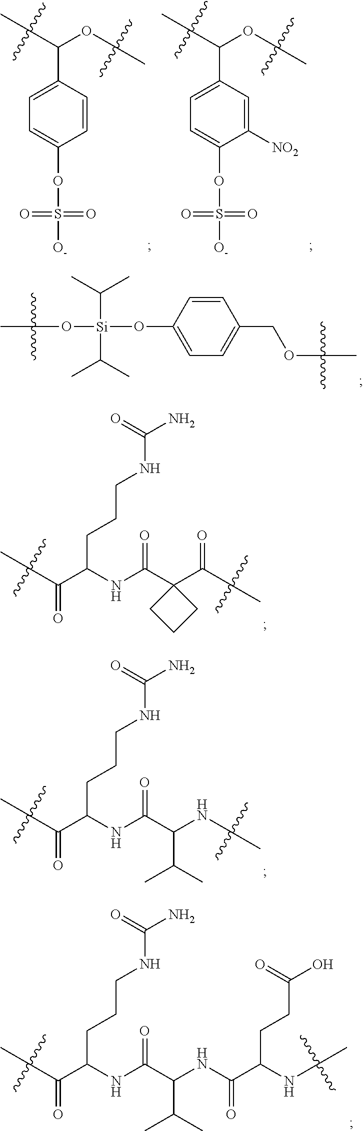

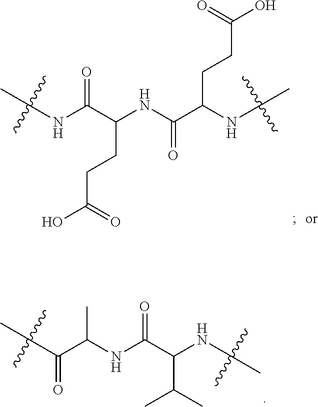 [SMILES: *NC(=O)C(CCC(=O)O)NC(=O)C(CCC(=O)O)N* ; *NC(C(=O)NC(C)C(*)=O)C(C)C]

In some embodiments, an immolative element comprises para-aminobenzyloxycarbonyl, an aminal, a hydrazine, a disulfide, an amide, an ester, a hydrazine, a phosphotriester, a diester, a 3-glucuronide, a double bond, a triple bond, an ether bond, a ketone, a diol, a cyano, a nitro, a quaternary amine, or combinations thereof. In certain embodiments, an immolative element comprises a paramethoxybenzyl, a dialkyldialkoxysilane, a diaryldialkoxysilane, an orthoester, an acetal, an optionally substituted β-thiopropionate, a ketal, a phosphoramidate, a hydrazone, a vinyl ether, an imine, an aconityl, a trityl, a polyketal, a bis-arylhydrazone, a diazobenzene, a vivinal diol, a pyrophosphate diester, or combinations thereof.

In some embodiments, an immolative element comprises one of the following structures:

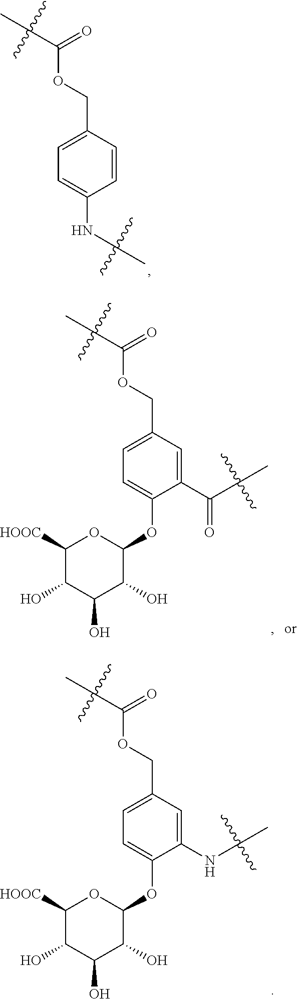 [SMILES: *Nc1ccc(COC(*)=O)cc1 ; *C(=O)OCc1ccc(O[C@@H]2O[C@H](C(=O)O)[C@@H](O)[C@H](O)[C@H]2O)c(C(*)=O)c1 ; *Nc1cc(COC(*)=O)ccc1O[C@@H]1O[C@H](C(=O)O)[C@@H](O)[C@H](O)[C@H]1O]

In certain embodiments, an immolative element comprises the following structure:

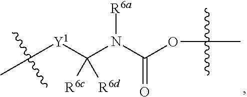

wherein:
R^{6a}, R^{6b}, R^{6c}, and R^{6d} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y¹ is —O—, —S—, or —NR^{6b}—.

In some embodiments, an immolative element comprises the following structure:

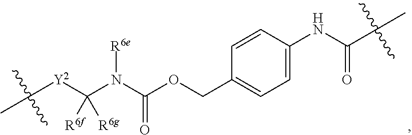

wherein:
R^{6e}, R^{6f}, R^{6g}, and R^{6h} are independently hydrogen, an optionally substituted alkyl, an optionally substituted aryl, or optionally substituted heteroaryl, or
R^{6a} and R^{6c} together with the nitrogen and carbon atoms to which they are attached form azetidinyl, pyrrolodinyl, piperidinyl, or homopiperidinyl and R^{6d} is hydrogen; and
Y² is —O—, —S—, or —NR^{6f}—.

In some embodiments, an immolative element comprises the following structure:

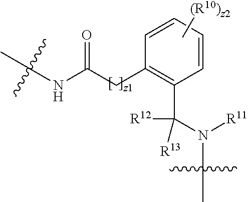

wherein:
each occurrence of R¹⁰ is independently alkyl, alkoxy, or halo;
R¹¹ is hydrogen, alkyl, or —(CH₂CH₂O)_{z3}—CH₃;
R¹² is hydrogen or alkyl;
R¹³ is hydrogen or alkyl;
z1 is 0 or 1;
z2 is 0, 1, 2, 3, or 4; and
z3 is 0, 1, 2, 3, 4, 5, 6, 7, 8, 9, or 10.

In some embodiments, an immolative element comprises one of the following structures:

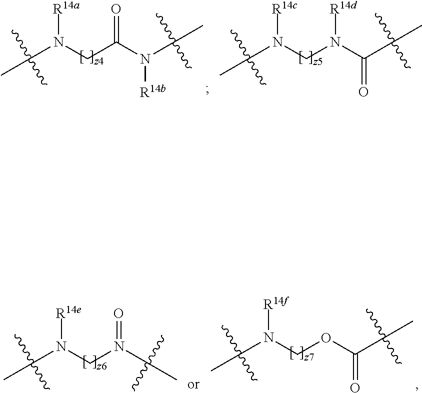

wherein:
R^{14a}, R^{14b}, R^{14c}, R^{14d}, R^{14e}, and R^{14f} are each independently hydrogen, alkyl, hydroxyalkyl, or alkoxyalkyl;
z4, z5, z6, and z7 are each independently 1, 2, 3, 4, 5, or 6.

In certain embodiments, an immolative element comprises one of the following structures:

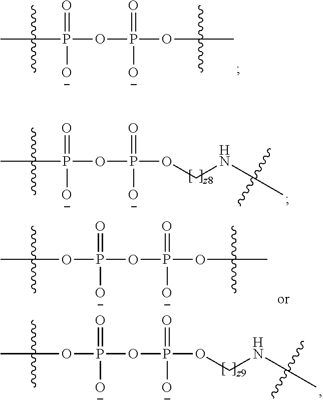

wherein:
z8 and z9 are each independently 1, 2, 3, 4, 5, or 6.

In some embodiments, an immolative element comprises one of the following structures:

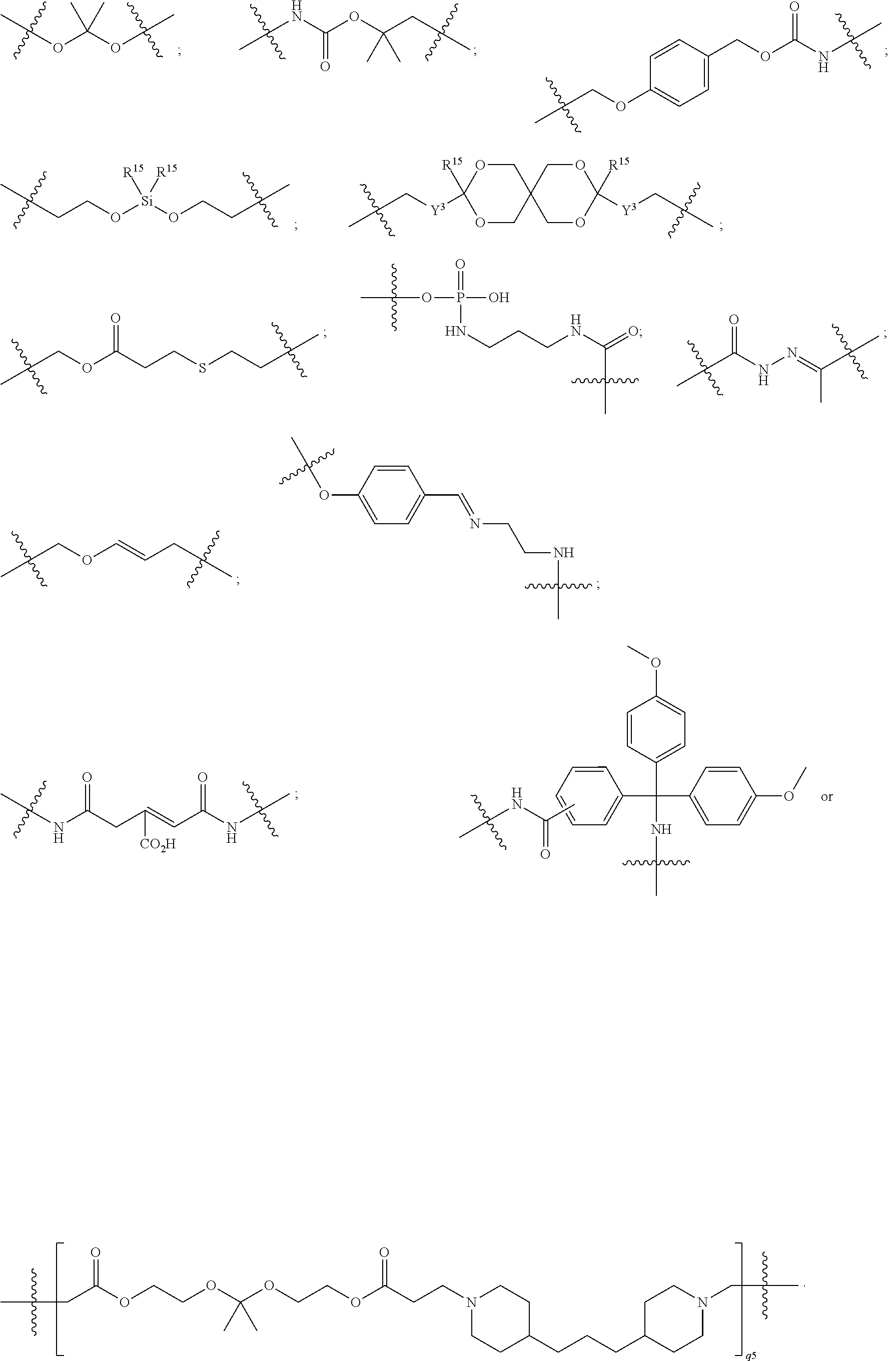

wherein:
each occurrence of R¹⁵ is independently H, methyl, ethyl, isopropyl, tert-butyl, or phenyl;
Y³ is O or CH₂; and
q5 is an integer ranging from 1-5.

In some embodiments, R^{4a} is hydrogen and [symbol] is a single bond. In some embodiments, R^{4a} is hydrogen and [symbol] is absent.

In some embodiments, a trigger element and an immolative element together comprise one of the following structures:

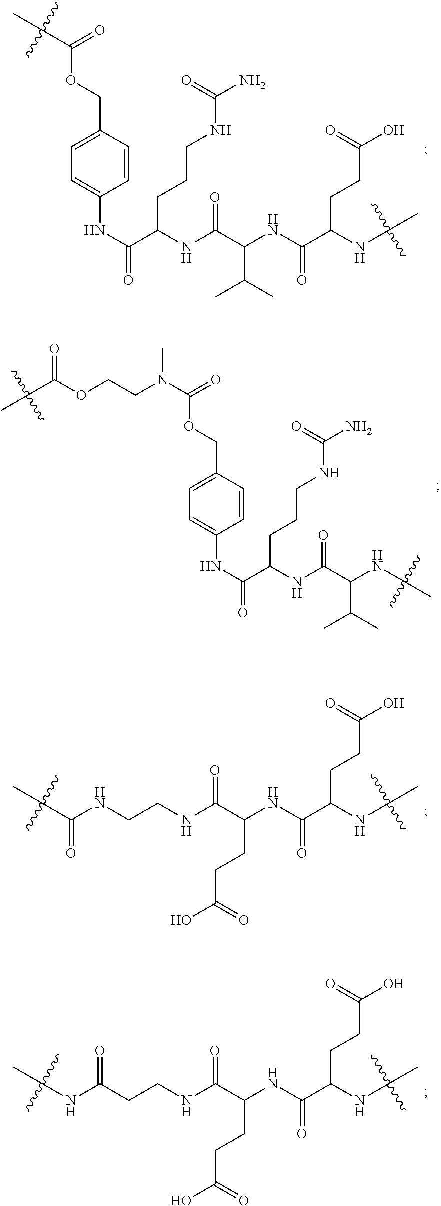 [SMILES: *NC(CCC(=O)O)C(=O)NC(C(=O)NC(CCCNC(N)=O)C(=O)Nc1ccc(COC(*)=O)cc1)C(C)C ; *NC(C(=O)NC(CCCNC(N)=O)C(=O)Nc1ccc(COC(=O)N(C)CCOC(*)=O)cc1)C(C)C ; *NC(CCC(=O)O)C(=O)NC(CCC(=O)O)C(=O)NCCNC(*)=O ; *NC(=O)CCNC(=O)C(CCC(=O)O)NC(=O)C(CCC(=O)O)N*]

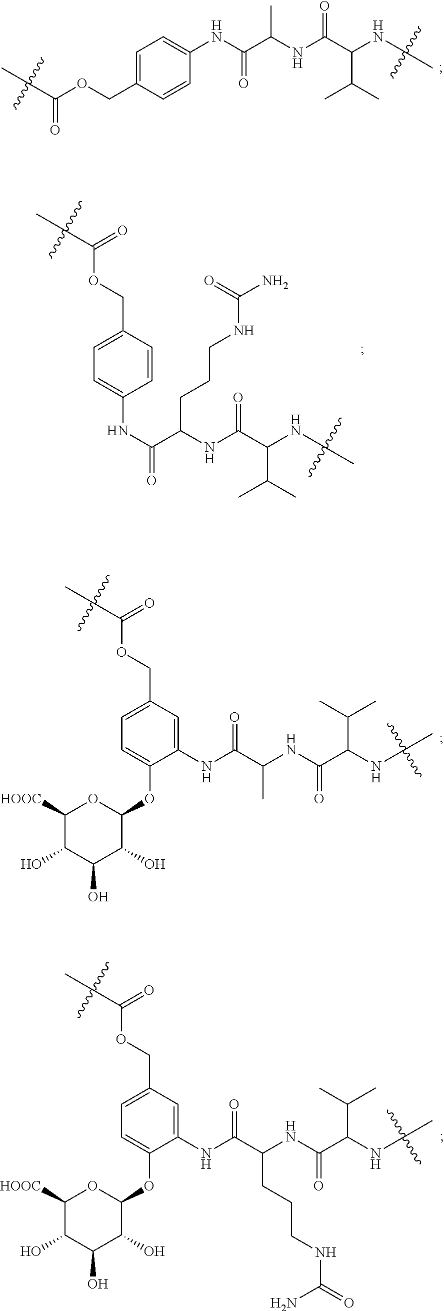 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

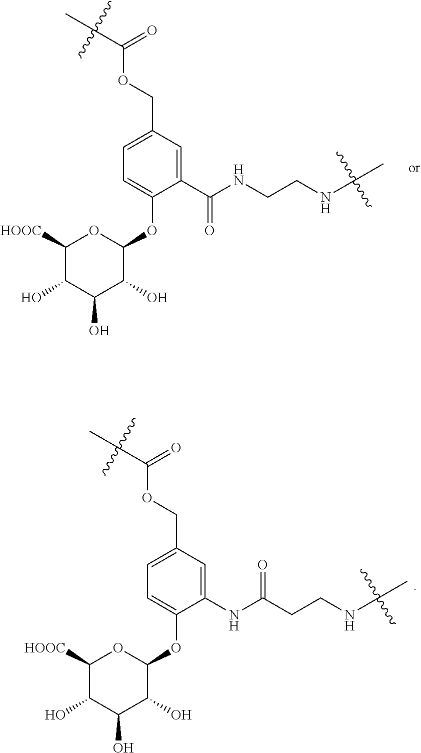 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O]

In some embodiments, a polar cap comprises one or more charged amino acid, one or more polyol, or combinations thereof. In certain embodiments, a polar cap comprises a diol, a triol, a tetraol, or combinations thereof. In some embodiments, a polar cap comprises glycerol, trimethylolpropane, pentaerythritol, maltitol, sorbitol, xylitol, erythritol, isomalt, or combinations thereof. In certain embodiments, a polar cap comprises one or more natural amino acids. In some embodiments, a polar cap comprises one or more non-natural amino acids. In certain embodiments, a polar cap comprises one or more non-natural amino acids and one or more natural amino acids. In some embodiments, a polar cap comprises serine, threonine, cysteine, proline, asparagine, glutamine, lysine, arginine, histidine, aspartate, glutamate, 4-hydroxyproline, 5-hydroxylysine, homoserine, homocysteine, ornithine, beta-alanine, statine, or gamma aminobutyric acid. In certain embodiments, a polar cap comprises aspartic acid, serine, glutamic acid, serine-beta-glucose, or combinations thereof.

In some embodiments, a polar cap has one of the following structures, including combinations thereof:

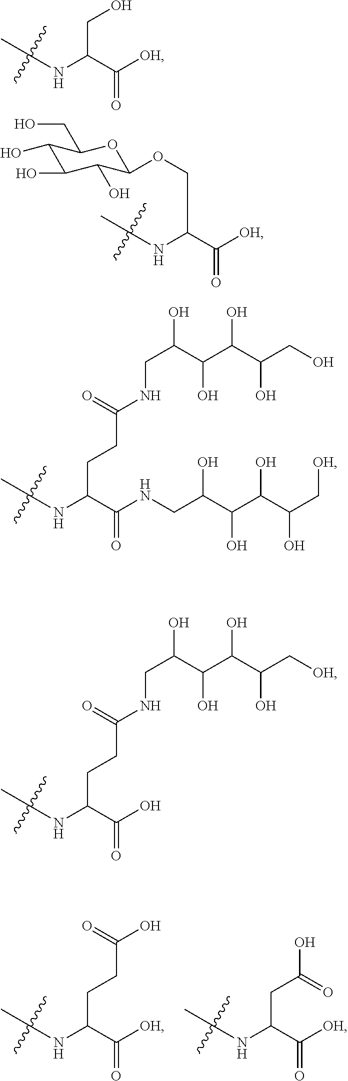

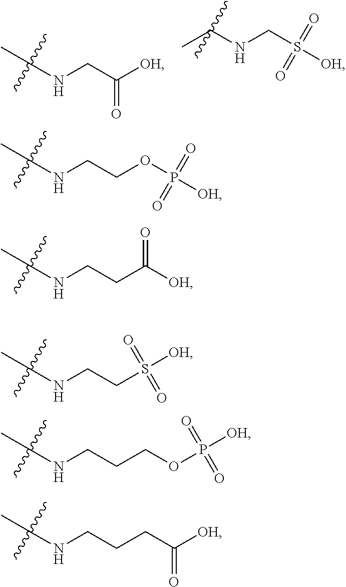

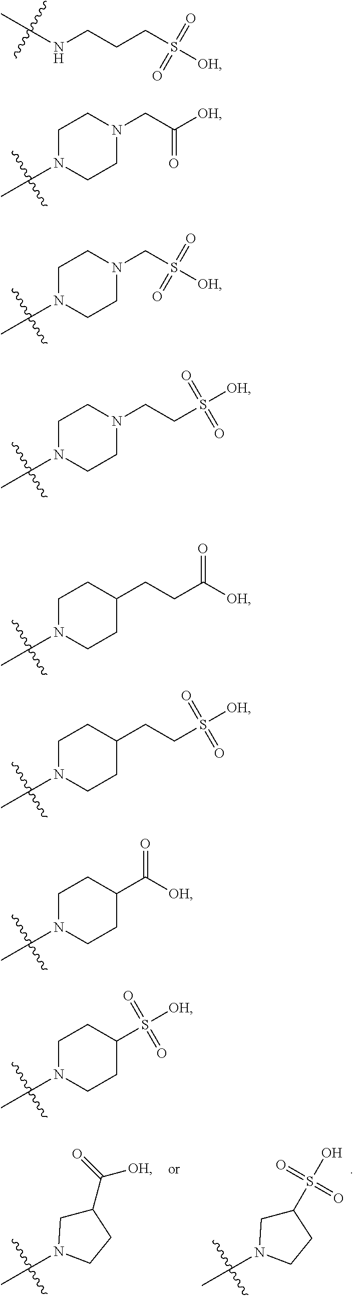

In some embodiments, L¹, L², or L³ comprise a linker selected from the group alkylene, alkylene-L^{a}-, alkenylene, alkenylene-L^{a}-, alkynylene, alkynylene-L^{a}-, -L^{a}-, -L^{a}-alkylene-L^{a}-, -L^{a}-alkenylene-L^{a}-, -L^{a}-alkynylene-L^{a}-, and combinations thereof, wherein each alkylene, alkenylene, and alkynylene is optionally substituted;
each occurrence of L^{a} is independently selected from —O—, —S—, —N(R⁷)—, —C(O)—, —C(S)—, —C(O)O—, —OC(O)—, —OC(O)O—, —C(O)N(R⁷)—, —N(R⁷)C(O)—, —C(O)N(R⁷)C(O)—, —C(O)N(R⁷)C(O)N(R⁷), —N(R⁷)C(O)N(R⁷)—, —N(R⁷)C(O)O—, —OC(O)N(R⁷)—, —C(NR⁷)—, —N(R⁷)C(NR⁷)—, —C(NR⁷)N(R⁷)—, —N(R⁷)C(NR⁷)N(R⁷)—, —S(O)₂—, —OS(O)—, —S(O)O—, —S(O), —OS(O)₂—, —S(O)₂O, —N(R⁷)S(O)₂—, —S(O)₂N(R⁷)—, —N(R⁷)S(O)—, —S(O)N(R⁷)—, —N(R⁷)S(O)₂N(R⁷)—, and —N(R⁷)S(O)N(R⁷)—;
R⁷ is independently selected at each occurrence from hydrogen, —NH₂, —C(O)OCH₂C₆H₅; and C₁₋₁₀ alkyl, C₂₋₁₀ alkenyl, C₂₋₁₀ alkynyl, C₃₋₁₂ cycloalkyl, and 3- to 12-membered heterocycle, each of which is optionally substituted with one or more substituents independently selected from halo, hydroxyl, cyano, nitro, amino, oxo, thioxo, —C(O)OCH₂C₆H₅, —NHC(O)OCH₂C₆H₅, C₁₋₁₀ alkyl, C₁₋₁₀ haloalkyl, C₁₋₁₀ alkoxy, C₂₋₁₀ alkenyl, C₂₋₁₀ alkynyl, C₃₋₁₂ cycloalkyl, and 3- to 12-membered heterocyclyl.

In some embodiments, each L¹, L², or L³ is optionally substituted with one or more substituents selected from alkyl, alkenyl, alkynyl, halo, hydroxyl, cyano, —OR⁸, —SR⁸, amino, aminyl, amido, cycloalkyl, aryl, heterocyclyl, heteroaryl, cycloclkylalkyl, arylalkyl, heterocyclylalkyl, heteroarylalkyl, —C(O)R⁸, —C(O)N(R⁸)₂, —N(R⁸)C(O)R⁸, —C(O)OR⁸, —OC(O)R⁸, —S(O)R⁸, —S(O)₂R⁸, —P(O)(OR⁸)₂, —OP(O)(OR⁸)₂, nitro, oxo, thioxo, ═N(R⁸), or cyano; and
R⁸ is hydrogen, alkyl, alkenyl, alkynyl, cycloalkyl, aryl, heterocyclyl, heteroaryl, cycloclkylalkyl, arylalkyl, heterocyclylalkyl, or heteroarylalkyl.

In some embodiments, L¹, L², or L³ are independently selected from the following structures:

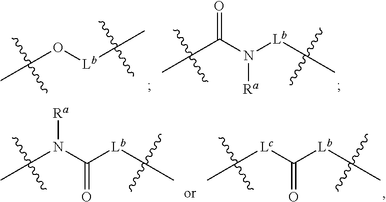

wherein:
   R^{a} is hydrogen or alkyl;
   each occurrence of L^{b} is independently a direct bond, an optionally substituted alkylene linker, an optionally substituted heteroalkylene linker, a heteroatomic linker, or a combination thereof,
   each occurrence of L^{c} is independently an optionally substituted alkylene linker;
provided that at least one of L¹, L², or L³ has the following structure:

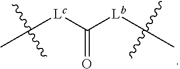

In some embodiments, L¹ and L² are independently selected from the following structures:

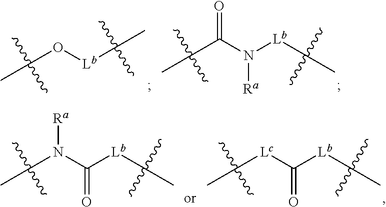

wherein:
   R^{a} is hydrogen or alkyl;
   each occurrence of L^{b} is independently a direct bond, an optionally substituted alkylene linker, an optionally substituted heteroalkylene linker, a heteroatomic linker, or a combination thereof,
   each occurrence of L^{c} is independently an optionally substituted alkylene linker;
provided that at least one of L¹ or L² has the following structure:

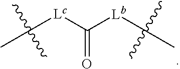

In some embodiments, L² has the following structure:

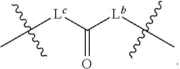

In some embodiments, L¹, L², and L³ each independently have one of the following structures:

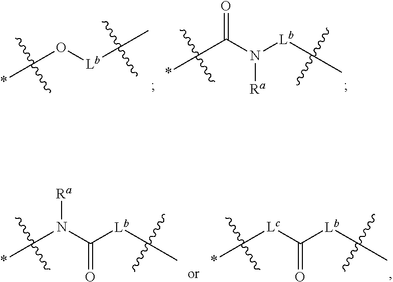

wherein:
   * indicates a direct bond to a substitutable position on the phenyl group of following structure:

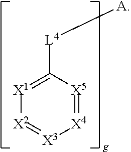

In some embodiments, L¹ or L² has one of the following structures:

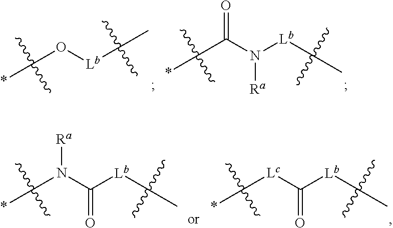

wherein:
   * indicates a direct bond to a substitutable position on the phenyl group of following structure:

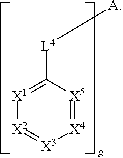

In some embodiments, L^{c} is C₁-C₆ alkylene.

In some embodiments, L^{c} is substituted with one or more substituents selected from the group consisting of halo, haloalkyl, alkoxy, cyano, nitro, carboxy, sulfonamide, sulfonic acid, or combinations thereof. In some embodiments, L^{c} is unsubstituted.

In some embodiments, the conjugate has one of the following Structures (IIxa) or (IIxb):

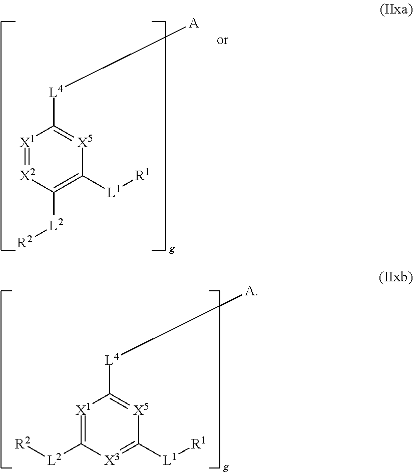

In some embodiments, the conjugate has one of the following Structures (IIxc′) or (IIxc″):

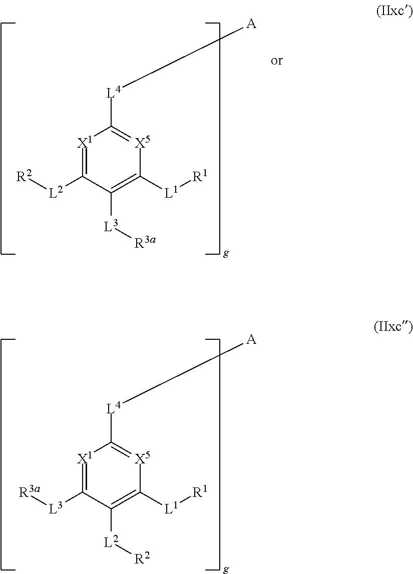

In certain embodiments, X², X³, or both are C—H, or C—F. In some embodiments, X¹, X⁵, or both are C—R³ and R³ is H or halo. In some embodiments, X¹ and X⁵ are both C—R³ and R³ is H or halo. In some embodiments, X¹ is C—R³ and R³ is halo. In certain embodiments, X⁵ is C—R³ and R³ is halo. In some embodiments, halo is fluoro. In some embodiments, X¹ is C—F. In some embodiments, X¹ is C—H. In some embodiments, X⁵ is C—H. In some embodiments, X⁵ is C—F. In some embodiments, X³ is C—R³ and R³ is H. In some embodiments, X³ is C—R³ and R³ is halo (e.g., fluoro).

In some embodiments, the conjugate has one of the following structures (IIIa-1), (IIxa-2), (IIxa-3), (IIxa-4), (IIxa-5), or (IIxa-6):

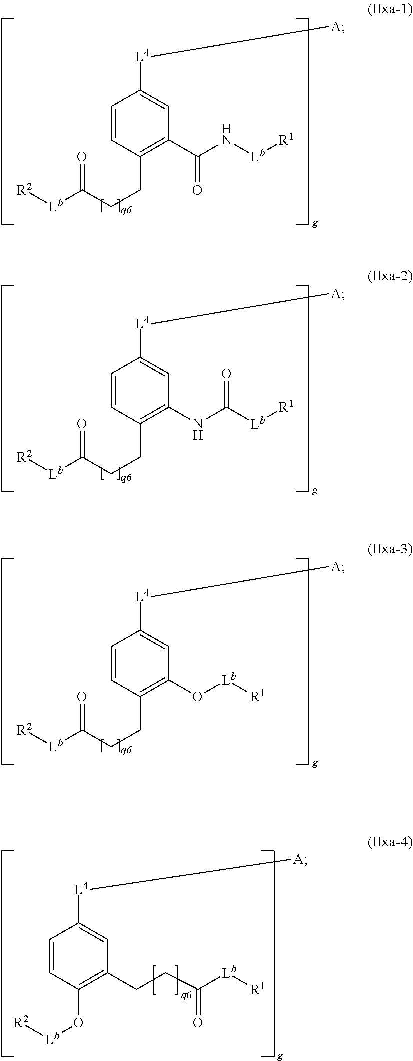

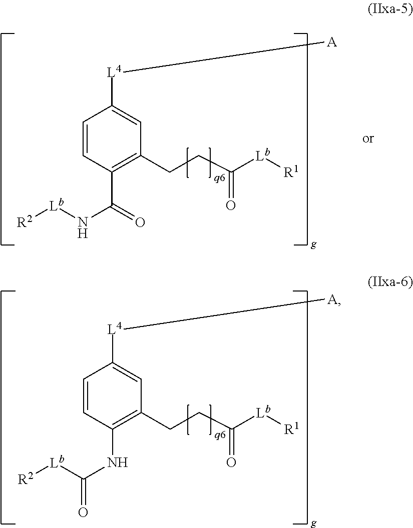

wherein:
   each occurrence of L^{b} is independently a direct bond, an optionally substituted alkylene linker, an optionally substituted heteroalkylene linker, a heteroatomic linker, or combinations thereof, and
   q6 is 0, 1, or 2.

In some embodiments, the conjugate has one of the following structures (IIxa-1), (IIxa-2), (IIxa-3), (IIxa-4), (IIxa-5), (IIxa-6), (IIXa-7), and (IIxa-8):

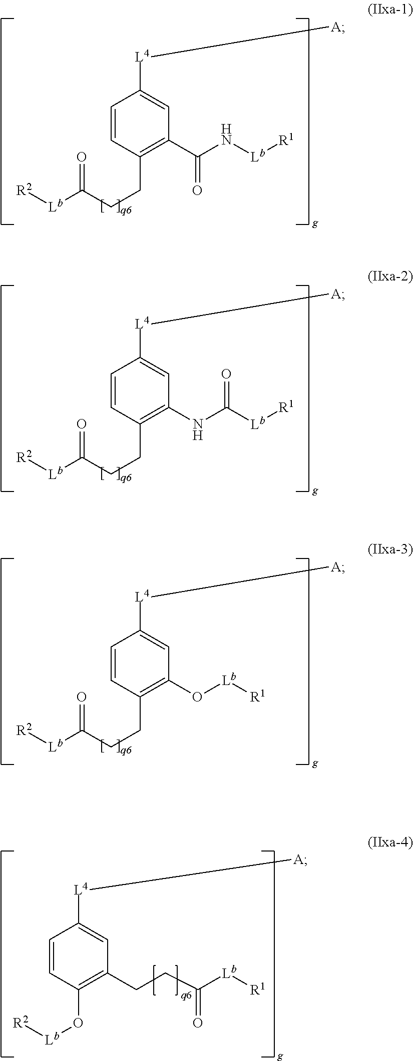

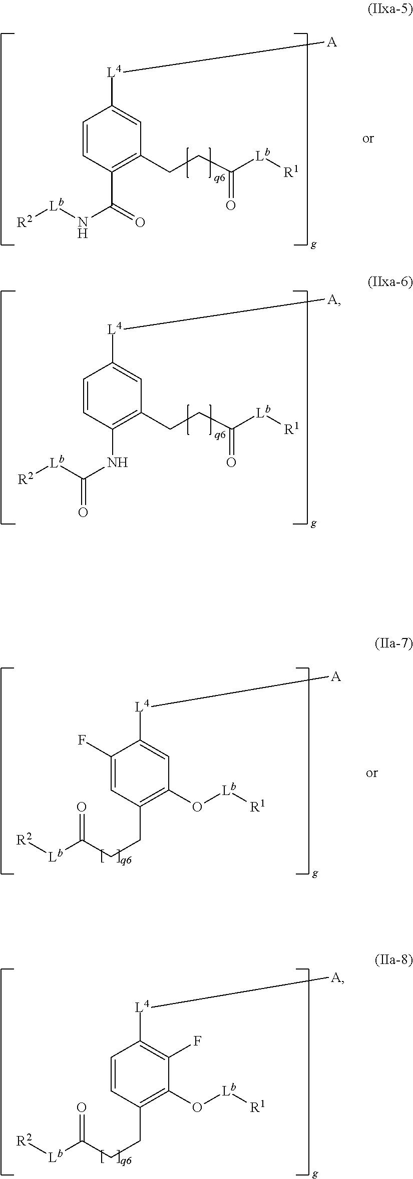

wherein:
   each occurrence of L^{b} is independently a direct bond, an optionally substituted alkylene linker, an optionally substituted heteroalkylene linker, a heteroatomic linker, or combinations thereof; and
   q6 is 0, 1, or 2.

In certain embodiments, the conjugate has one of the following Structures (IIxc-1), (IIxc-2), (IIxc-3), or (IIxc-4):

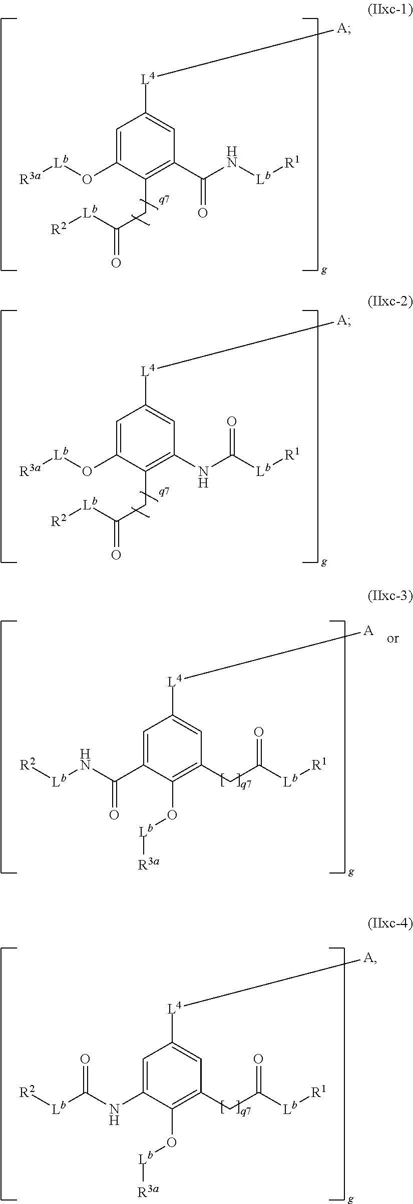

wherein:
   each occurrence of L^{b} is independently a direct bond, an optionally substituted alkylene linker, an optionally substituted heteroalkylene linker, a heteroatomic linker, or combinations thereof; and
   q7 is 1, 2, or 3.

In some embodiments, the conjugate has the following Structure (IIxd), (IIxe), (IIxf), or (IIxg):

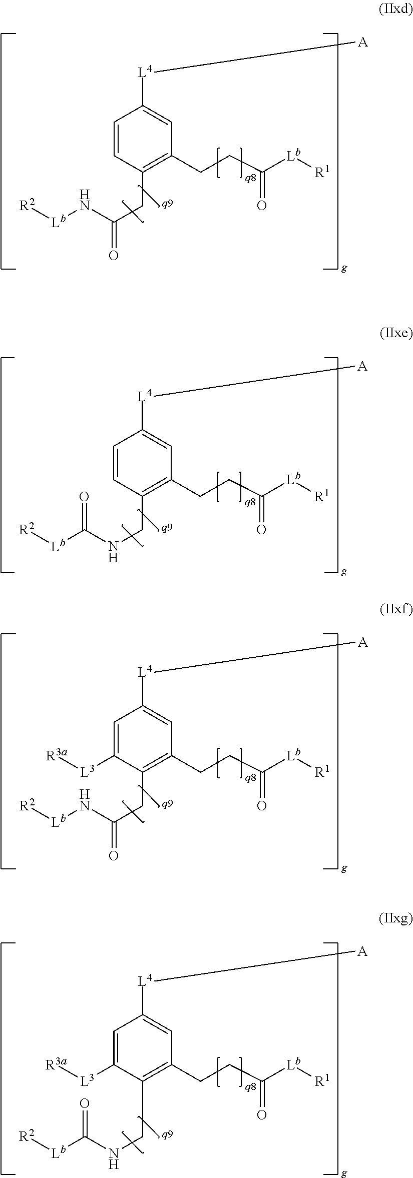

wherein:
   each occurrence of L^{b} is independently a direct bond, an optionally substituted alkylene linker, an optionally substituted heteroalkylene linker, a heteroatomic linker, or combinations thereof,
q8 is 0, 1, or 2; and
q9 is 0, 1, or 2.

In some embodiments, the conjugate has one of the following Structures (IIxh) or (IIxi):

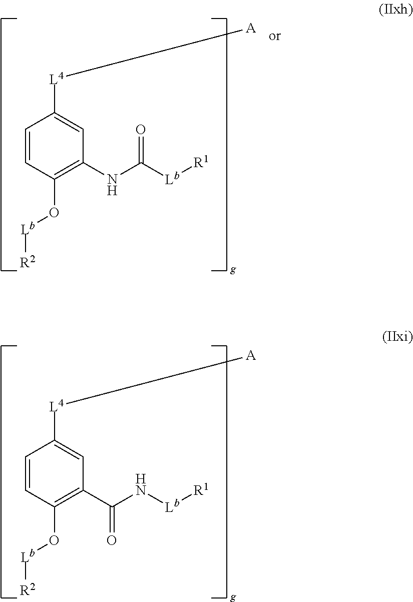

wherein:
   each occurrence of L^{b} is independently a direct bond, an optionally substituted alkylene linker, an optionally substituted heteroalkylene linker, a heteroatomic linker, or combinations thereof.

In some embodiments, L^{b} is a direct bond, an optionally substituted alkylene linker or an optionally substituted heteroalkylene linker.

In certain embodiments, L^{b} is a direct bond or has one of the following structures:

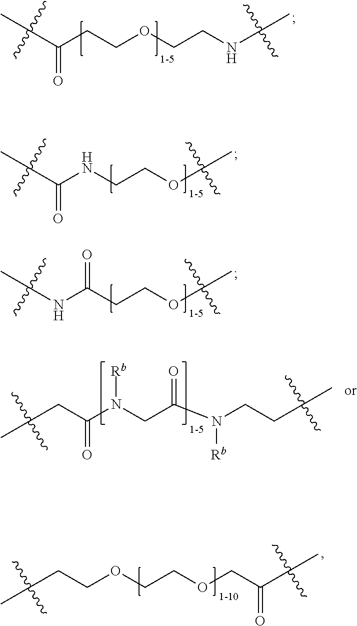

wherein:
each occurrence of R^{b} is independently hydrogen, alkyl, hydroxyalkyl, or alkoxyalkyl.

In some embodiments, the conjugate has one of the following structures:

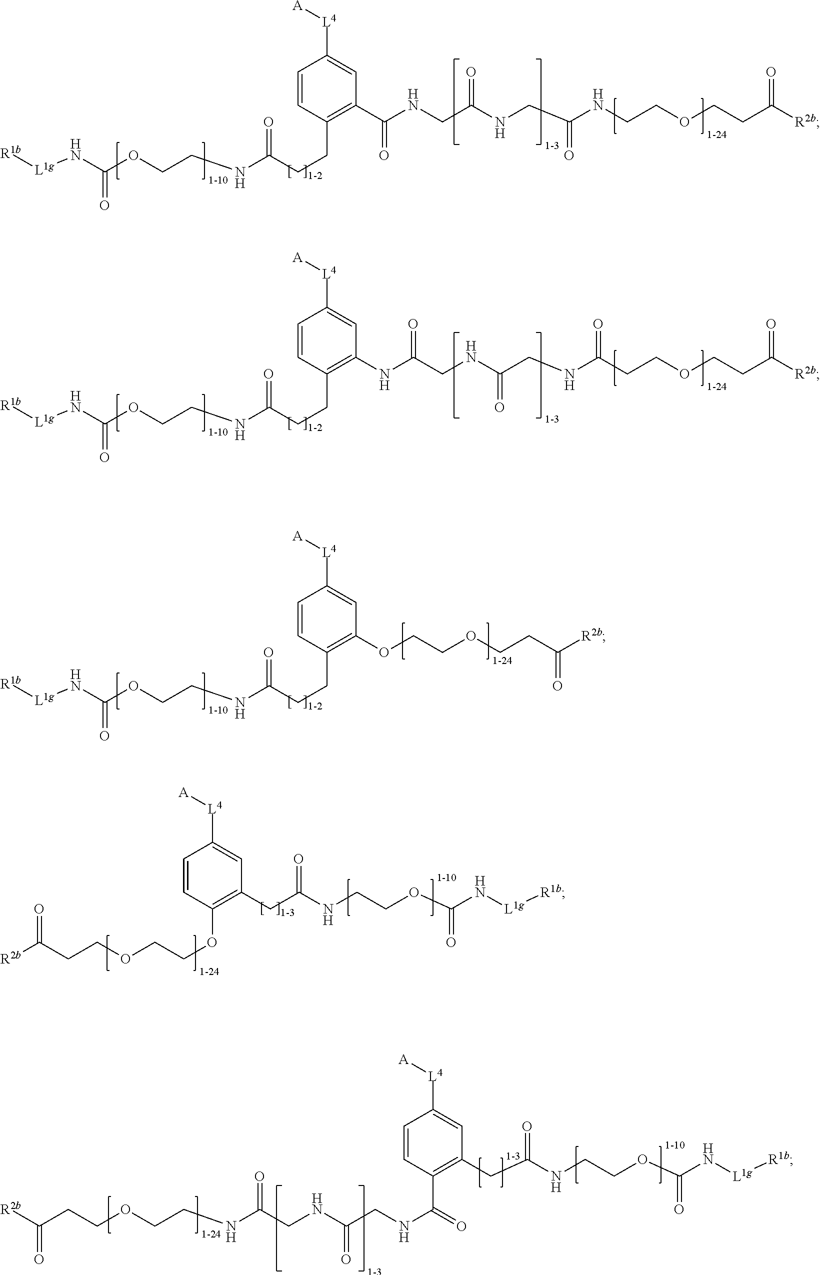

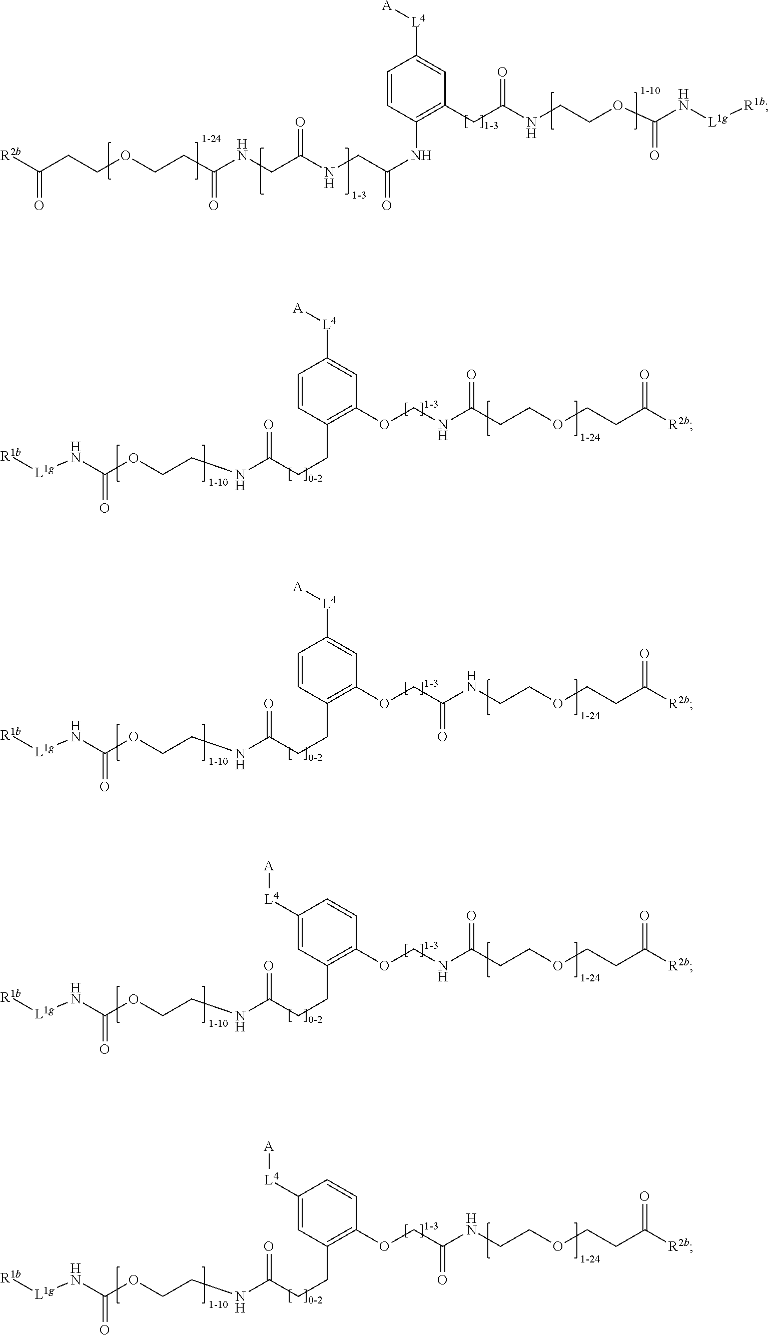

wherein:
R^{1b} is a chemotherapeutic, a cytotoxic agent, or a myeloid cell agonist;
R^{2b} has one of the following structures:

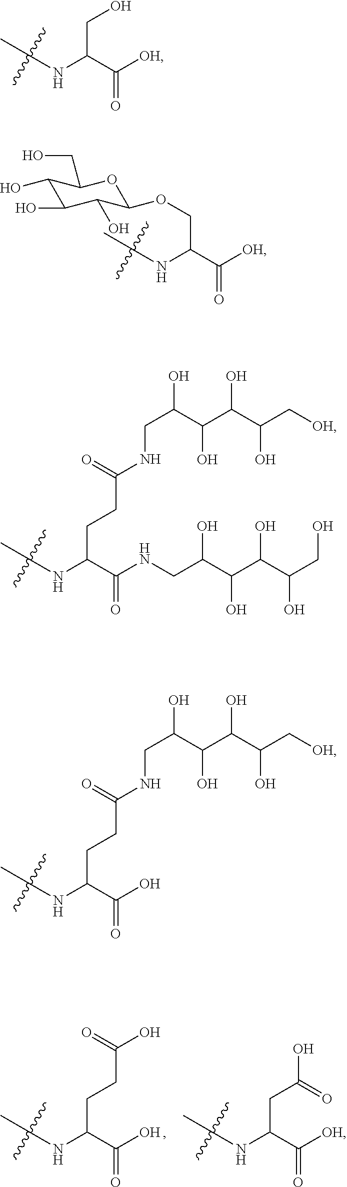 [SMILES: *NC(CO)C(=O)O ; *NC(COC1OC(CO)C(O)C(O)C1O)C(=O)O ; *NC(CCC(=O)NCC(O)C(O)C(O)C(O)CO)C(=O)NCC(O)C(O)C(O)C(O)CO ; *NC(CCC(=O)NCC(O)C(O)C(O)C(O)CO)C(=O)O ; *NC(CCC(=O)O)C(=O)O ; *NC(CC(=O)O)C(=O)O]

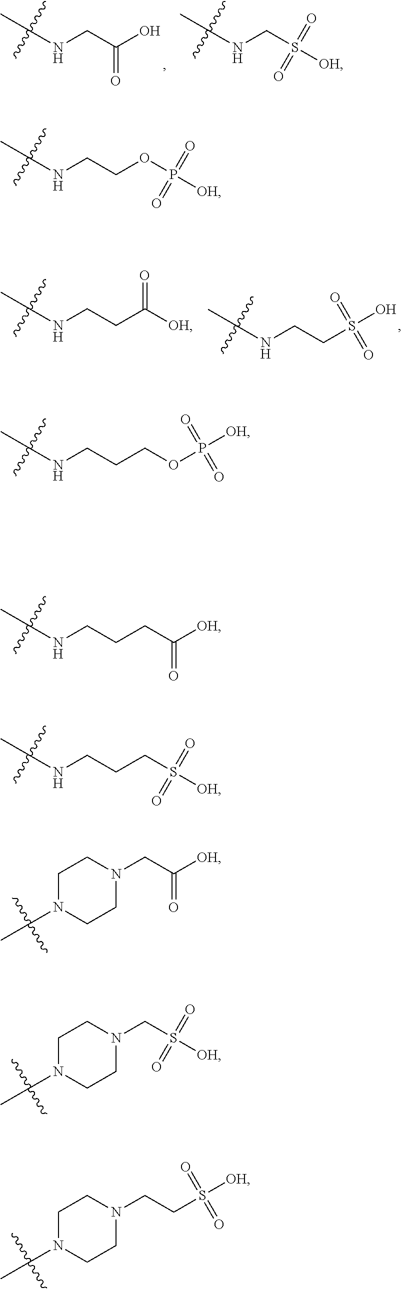

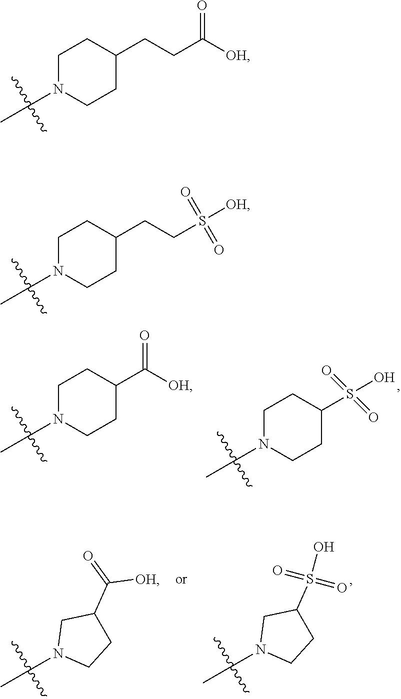 [SMILES: *N1CCC(CCC(=O)O)CC1 ; *N1CCC(CCS(=O)(=O)O)CC1 ; *N1CCC(C(=O)O)CC1 ; *N1CCC(S(=O)(=O)O)CC1 ; *N1CCC(C(=O)O)C1 ; *N1CCC(S(=O)(=O)O)C1]

L^{1g} has one of the following structures:

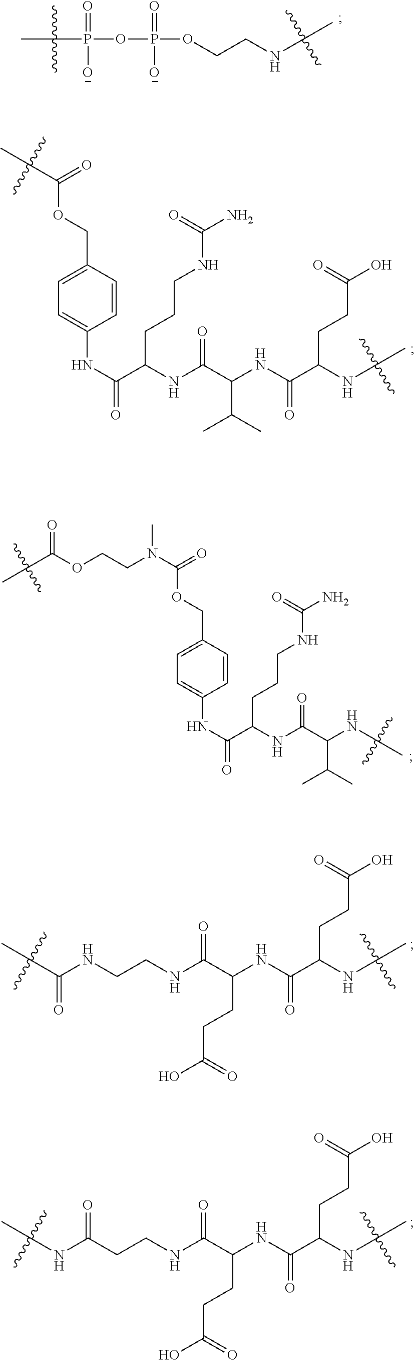 [SMILES: *NCCOP(=O)([O-])OP(*)(=O)[O-] ; *NC(CCC(=O)O)C(=O)NC(C(=O)NC(CCCNC(N)=O)C(=O)Nc1ccc(COC(*)=O)cc1)C(C)C ; *NC(C(=O)NC(CCCNC(N)=O)C(=O)Nc1ccc(COC(=O)N(C)CCOC(*)=O)cc1)C(C)C ; *NC(CCC(=O)O)C(=O)NC(CCC(=O)O)C(=O)NCCNC(*)=O ; *NC(=O)CCNC(=O)C(CCC(=O)O)NC(=O)C(CCC(=O)O)N*]

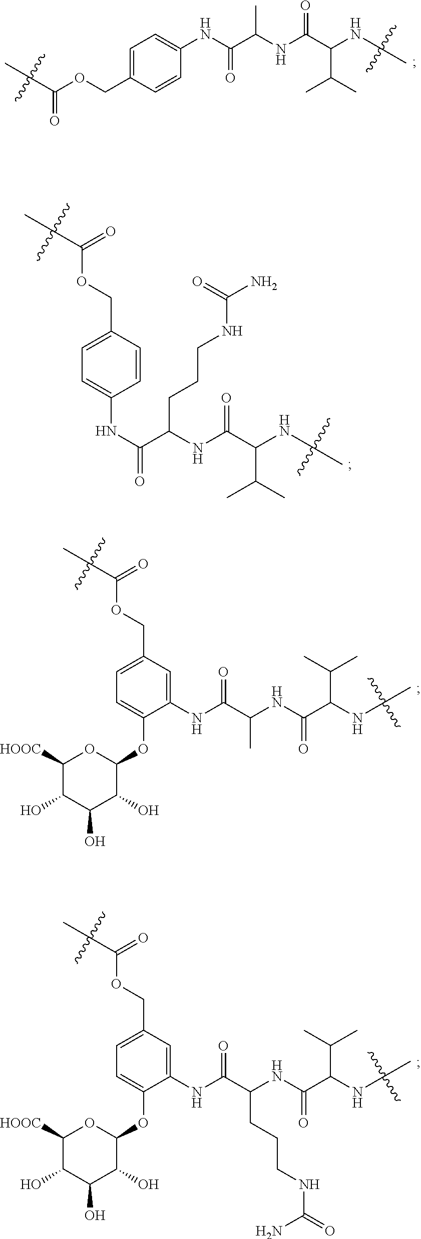 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

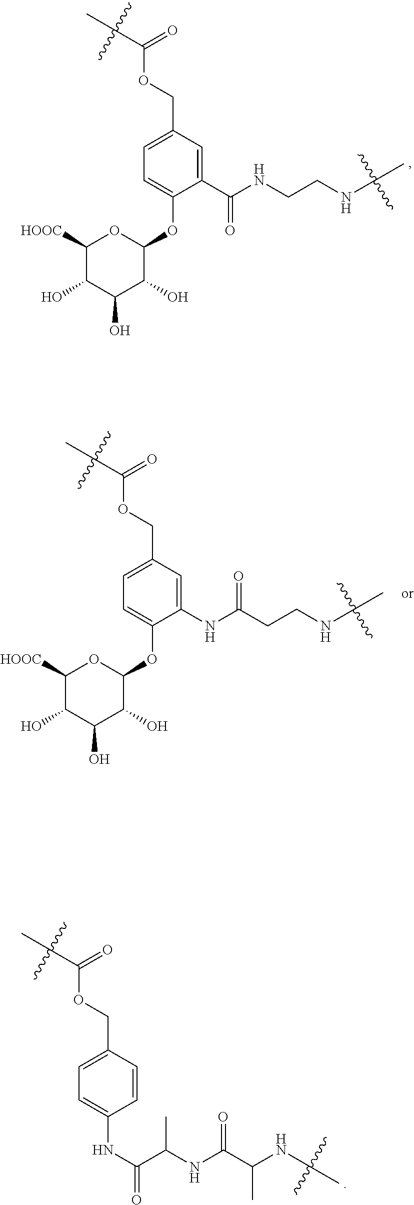 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the conjugate has one of the following structures:

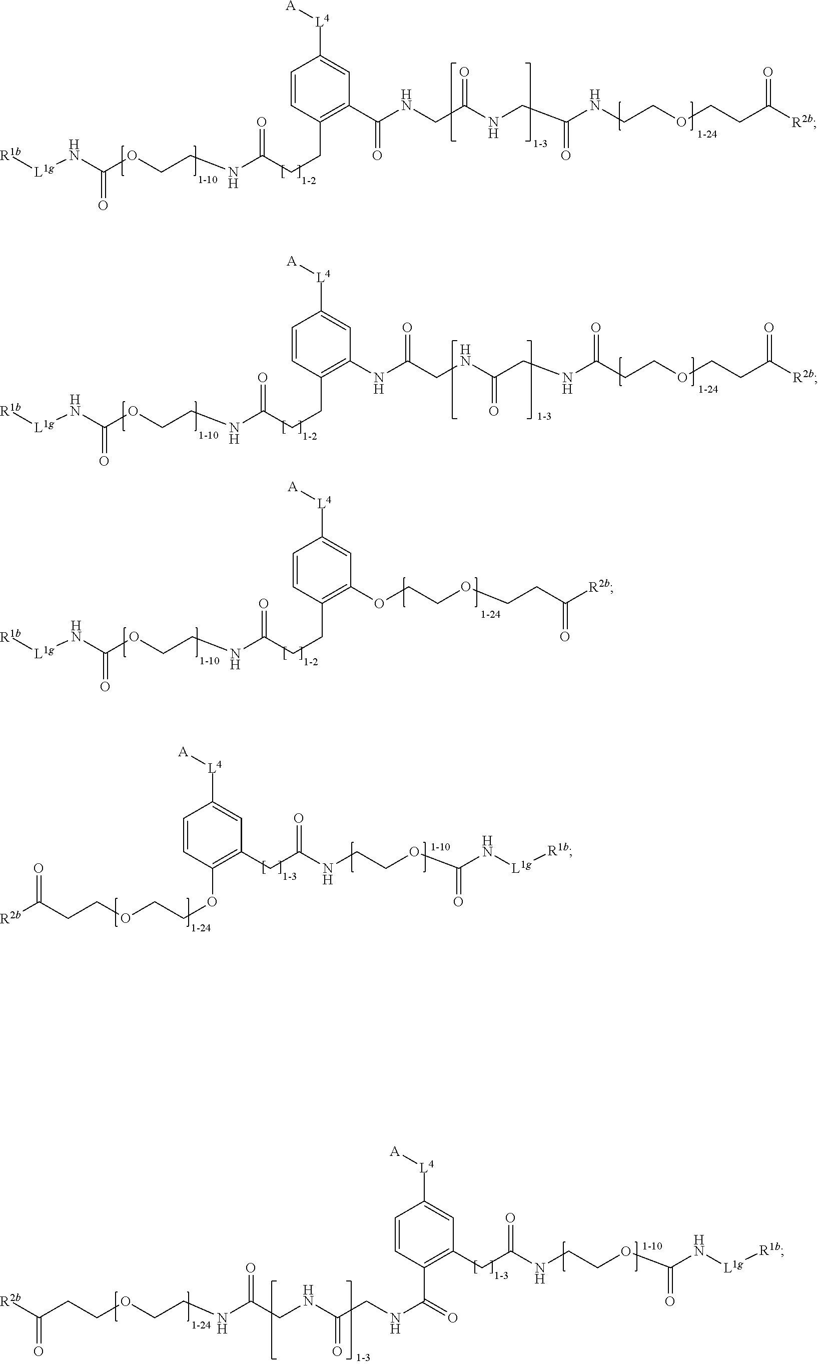

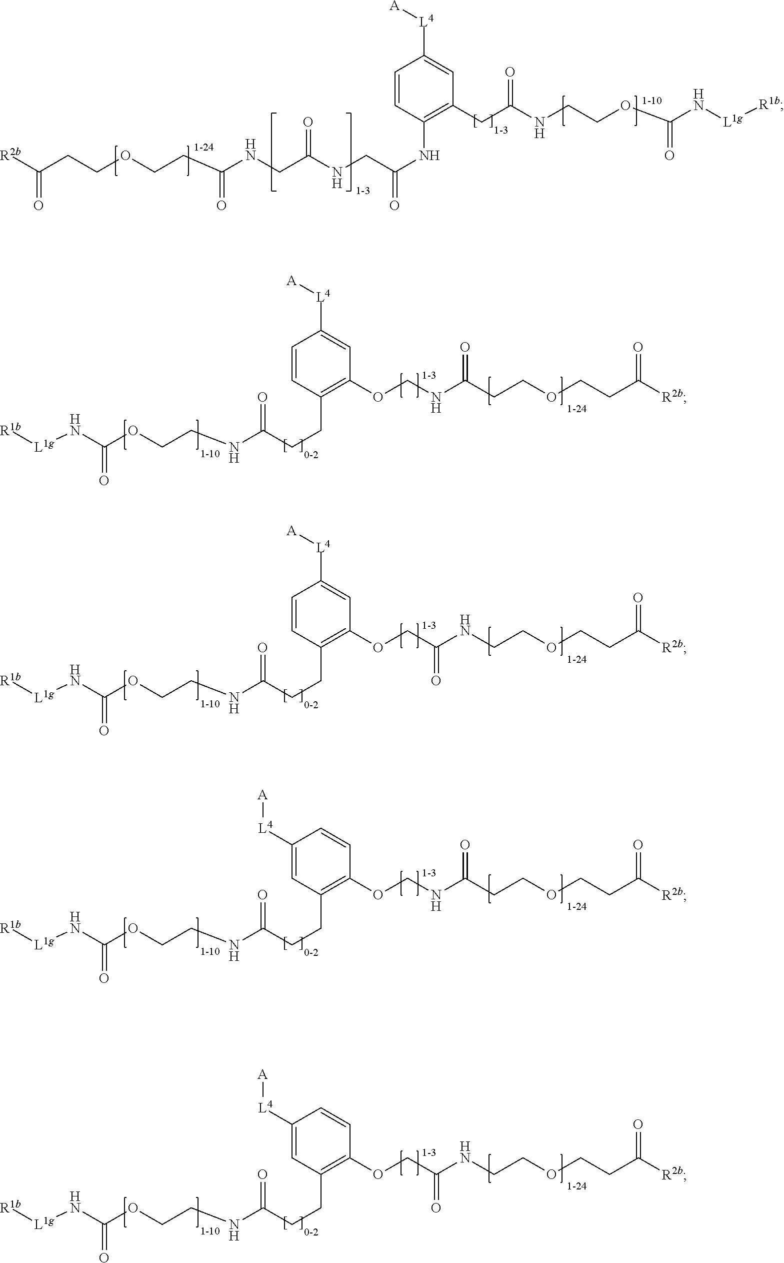

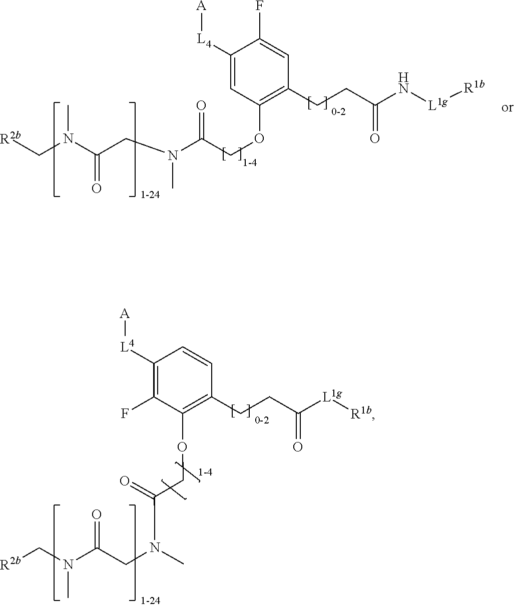

wherein:
   R^{2b} is a chemotherapeutic, a cytotoxic agent, or a myeloid cell agonist;
   R^{2b} has one of the following structures:

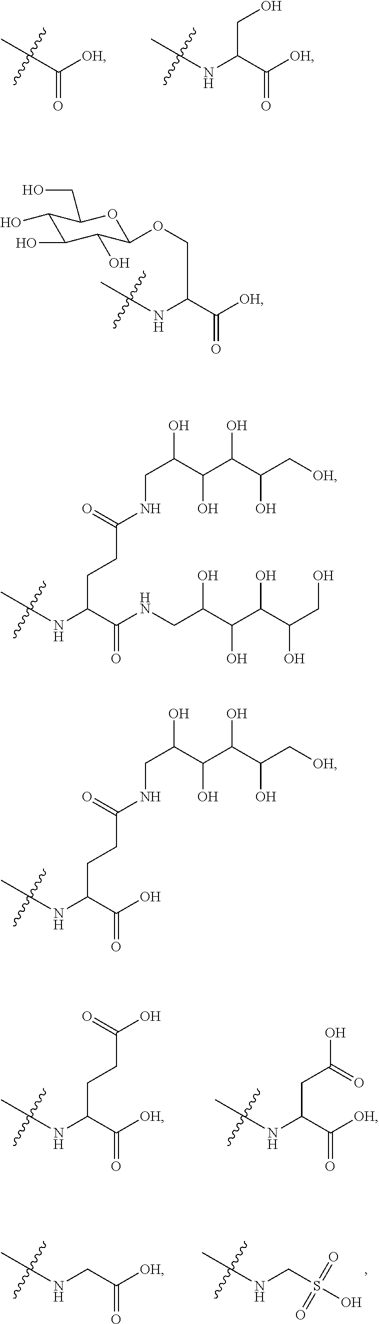

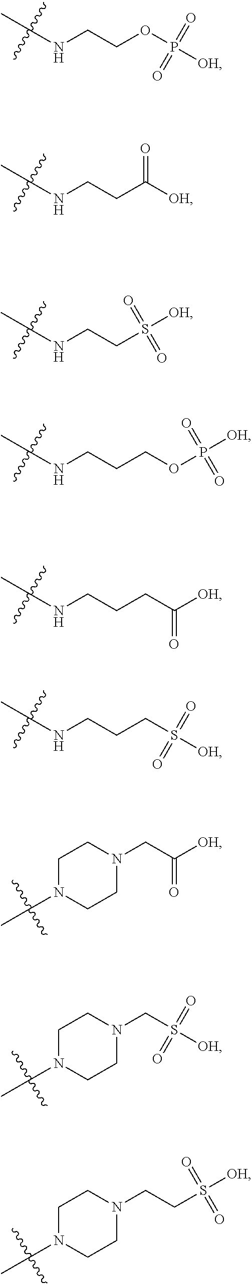

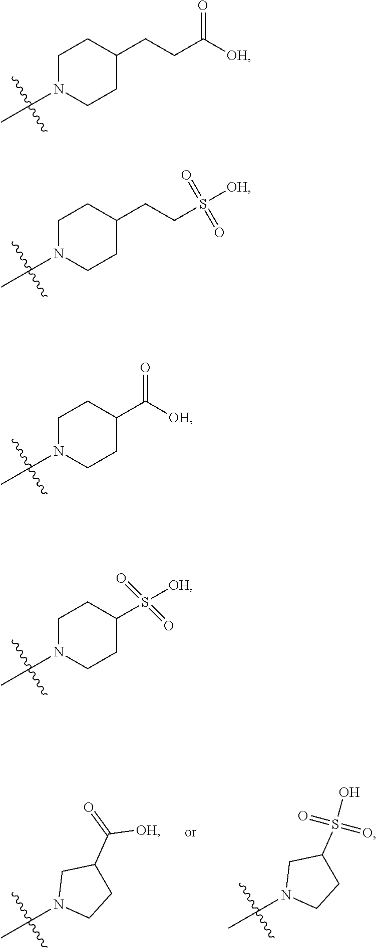 [SMILES: *N1CCC(CCC(=O)O)CC1 ; *N1CCC(CCS(=O)(=O)O)CC1 ; *N1CCC(C(=O)O)CC1 ; *N1CCC(S(=O)(=O)O)CC1 ; *N1CCC(C(=O)O)C1 ; *N1CCC(S(=O)(=O)O)C1]

L^{1g} has one of the following structures:

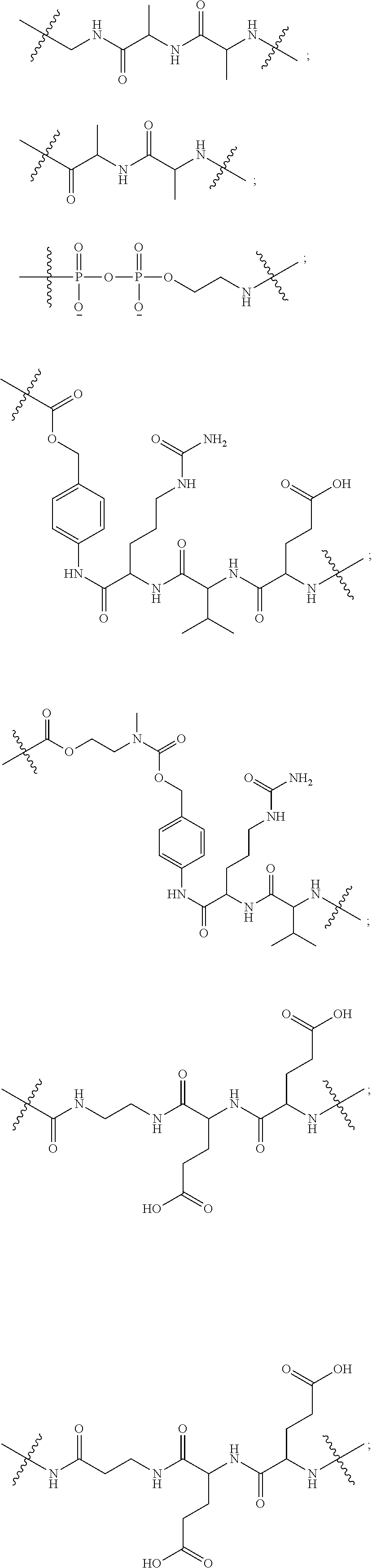

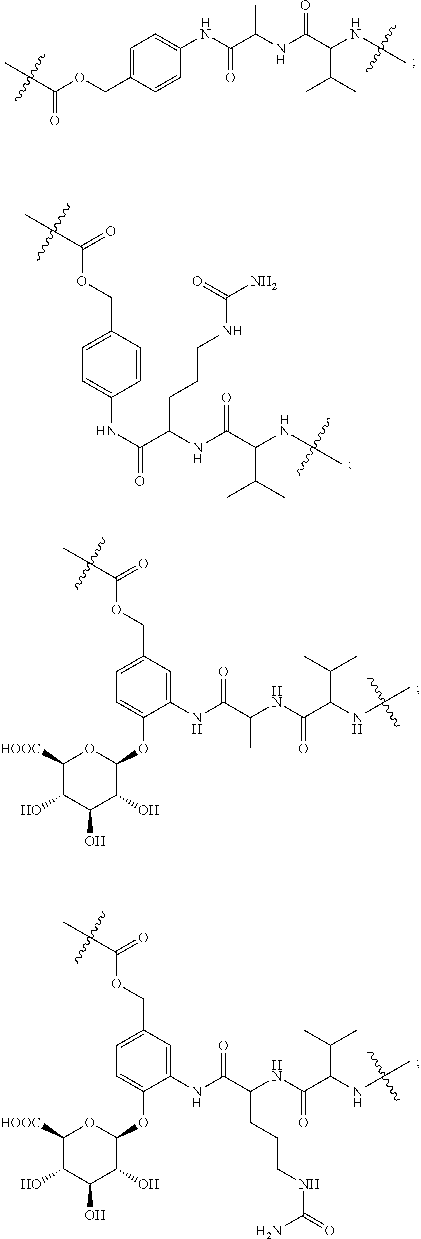 [SMILES: *NC(C(=O)NC(C)C(=O)Nc1ccc(COC(*)=O)cc1)C(C)C ; *NC(C(=O)NC(CCCNC(N)=O)C(=O)Nc1ccc(COC(*)=O)cc1)C(C)C ; *NC(C(=O)NC(C)C(=O)Nc1cc(COC(*)=O)ccc1O[C@@H]1O[C@H](C(=O)O)[C@@H](O)[C@H](O)[C@H]1O)C(C)C ; *NC(C(=O)NC(CCCNC(N)=O)C(=O)Nc1cc(COC(*)=O)ccc1O[C@@H]1O[C@H](C(=O)O)[C@@H](O)[C@H](O)[C@H]1O)C(C)C]

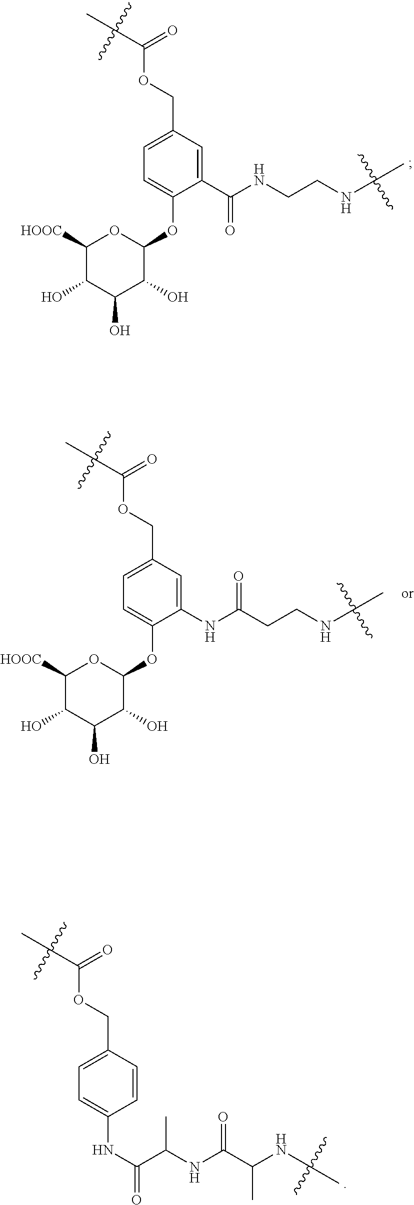 [SMILES: *NCCNC(=O)c1cc(COC(*)=O)ccc1O[C@@H]1O[C@H](C(=O)O)[C@@H](O)[C@H](O)[C@H]1O ; *NCCC(=O)Nc1cc(COC(*)=O)ccc1O[C@@H]1O[C@H](C(=O)O)[C@@H](O)[C@H](O)[C@H]1O ; *NC(C)C(=O)NC(C)C(=O)Nc1ccc(COC(*)=O)cc1]

In some embodiments, the conjugate has one of the following structures:

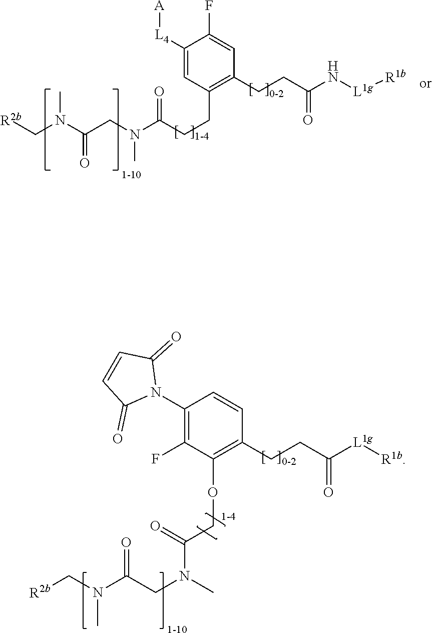

In some embodiments, the conjugate has one of the following structures:

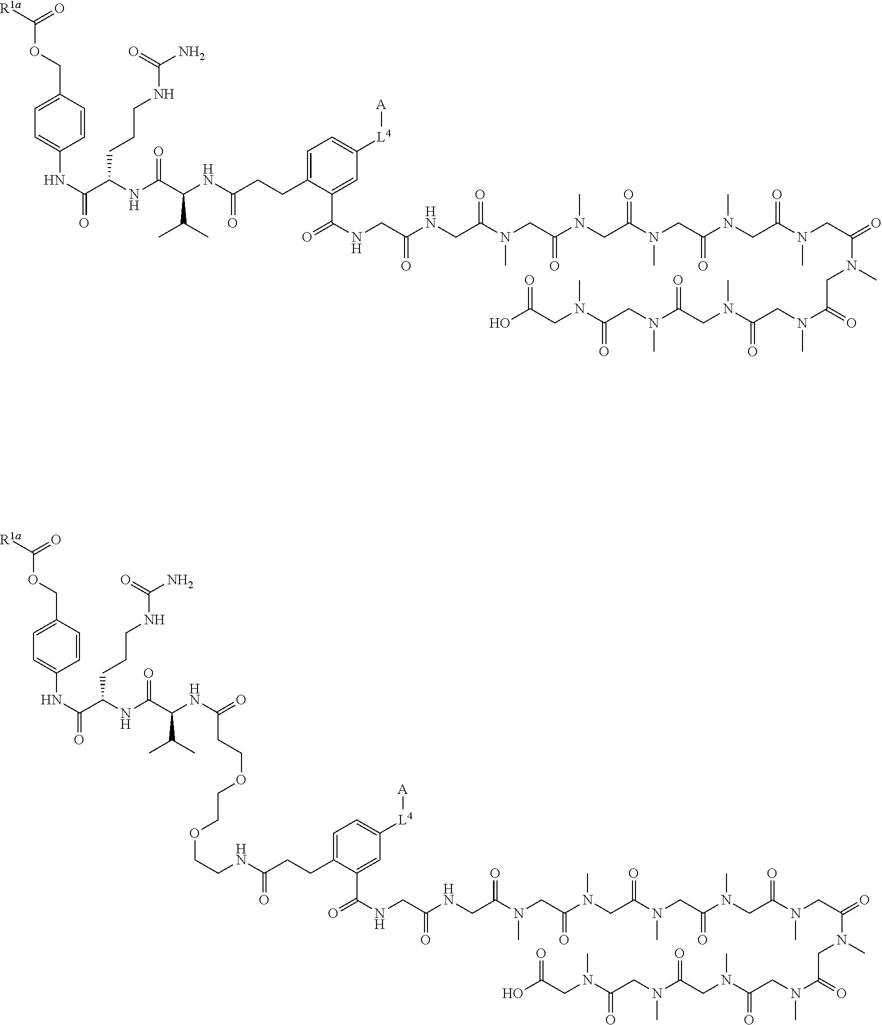

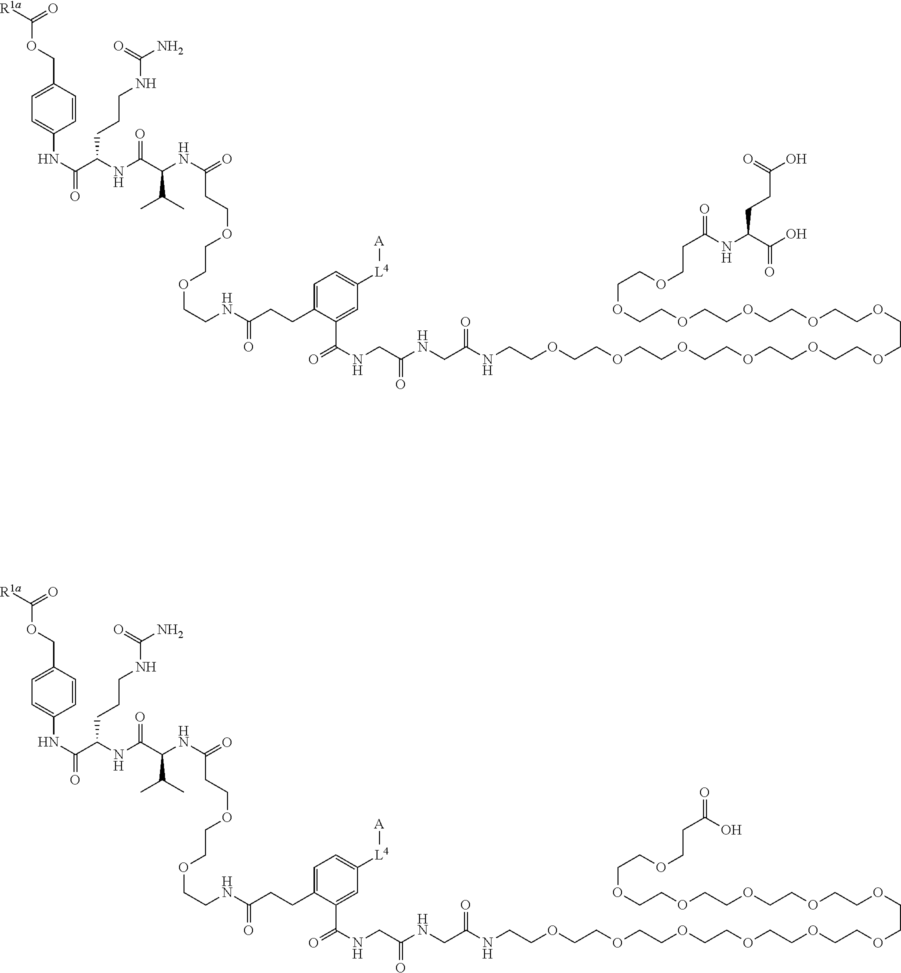

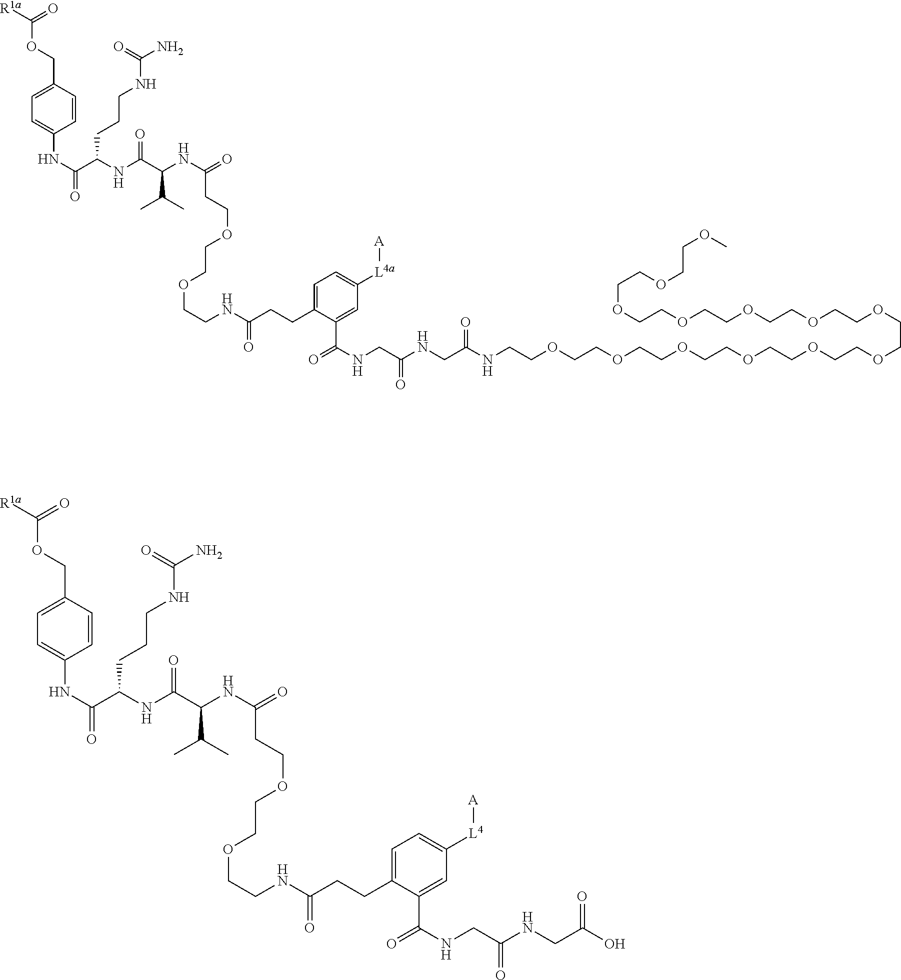

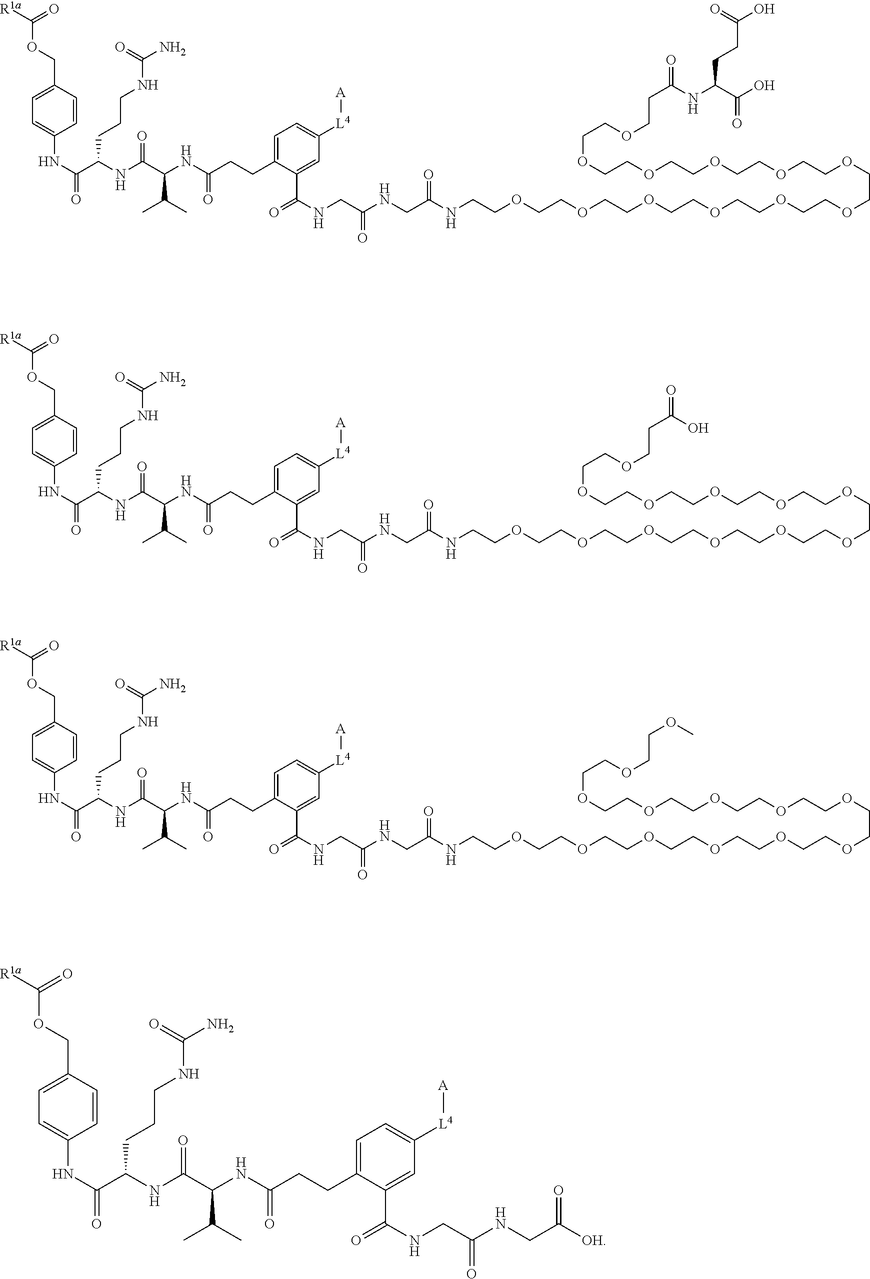

In certain embodiments, a conjugate has one of the following structures:

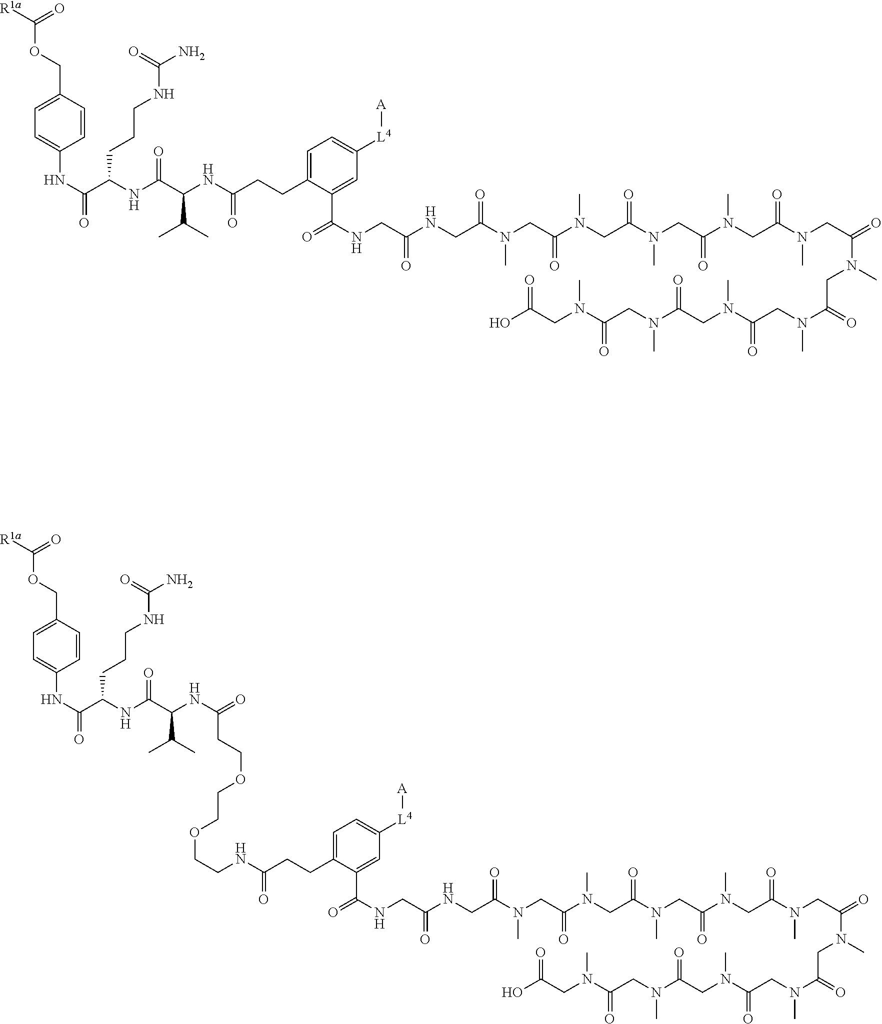

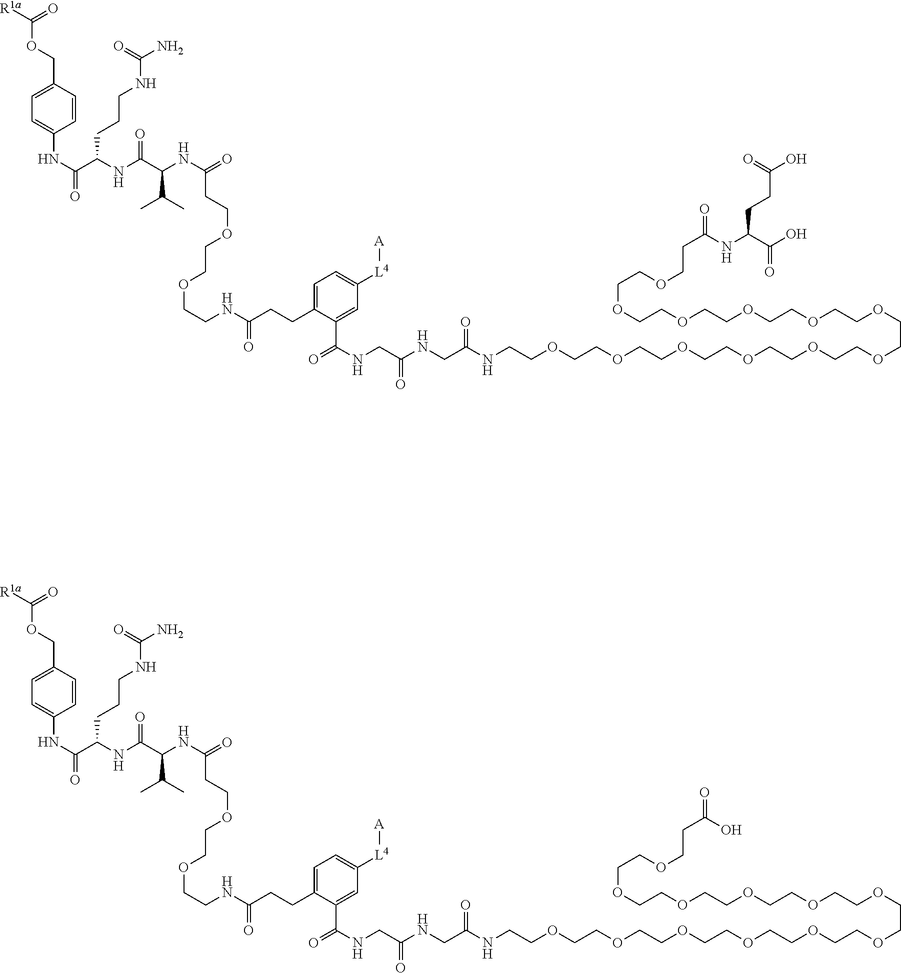

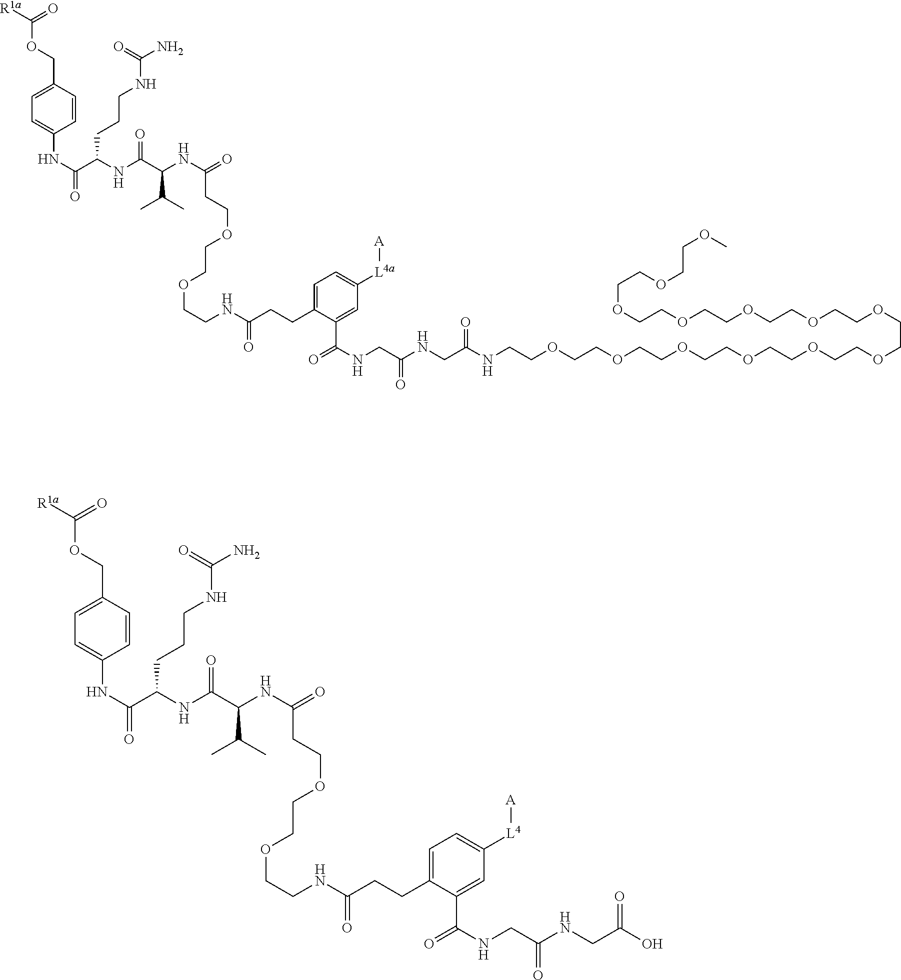

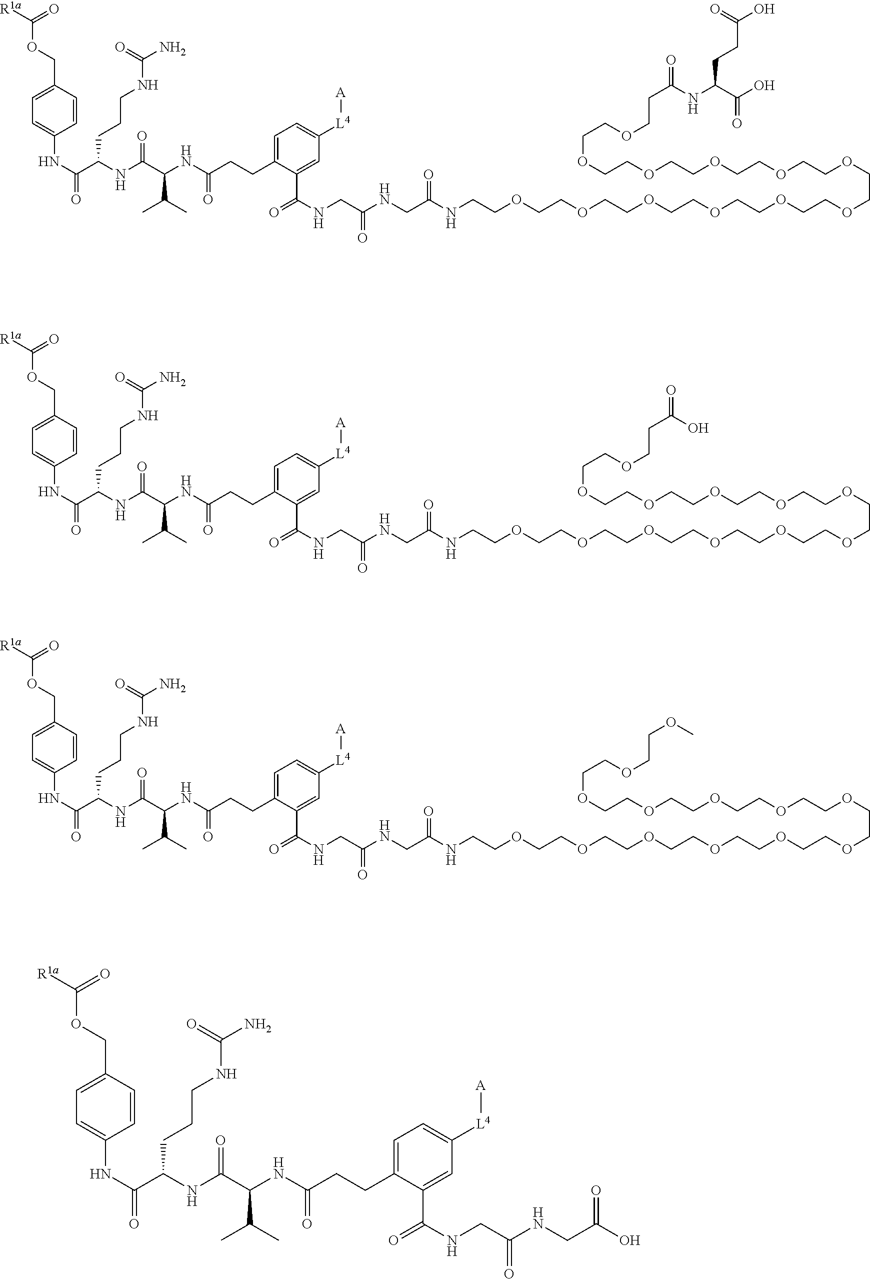

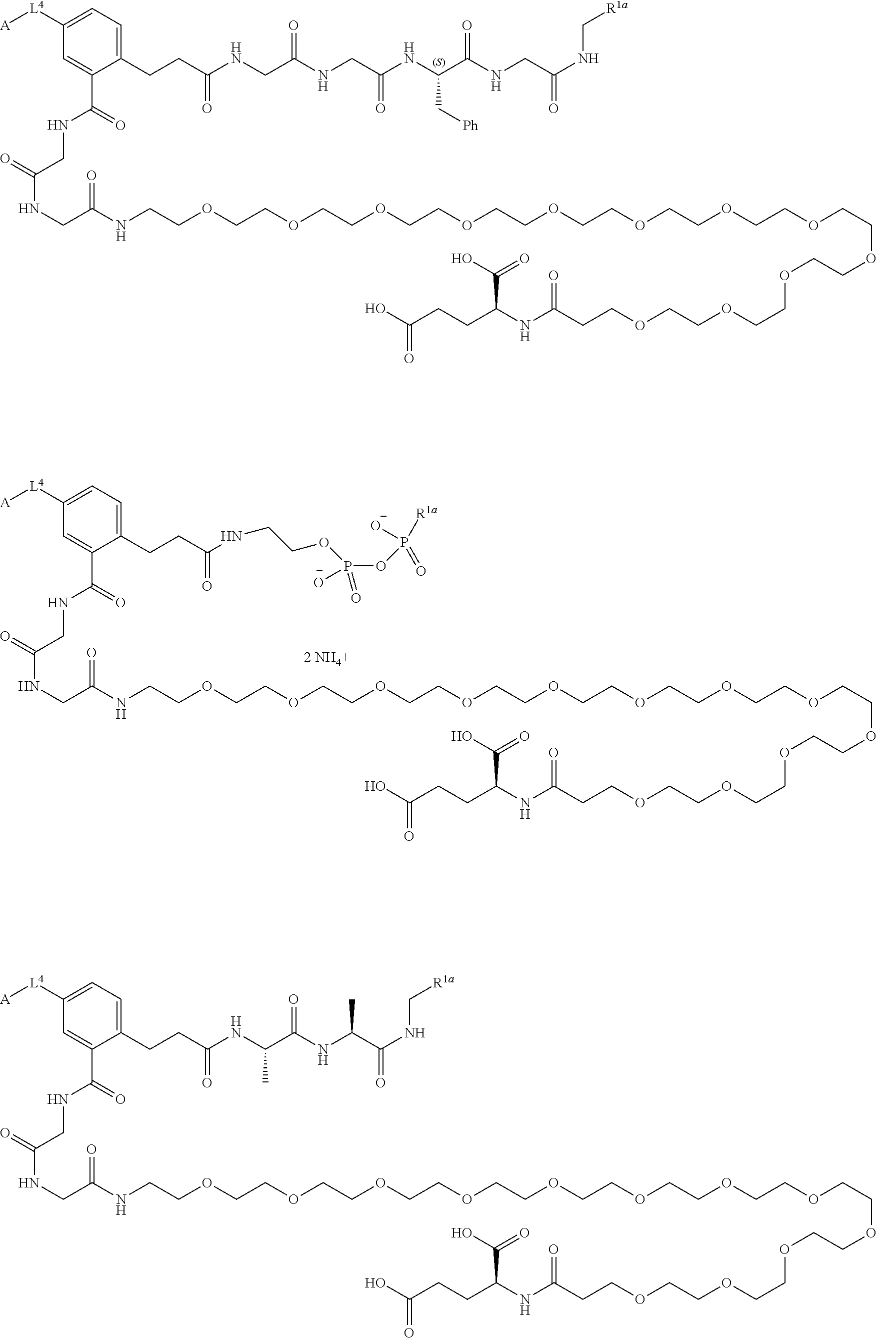

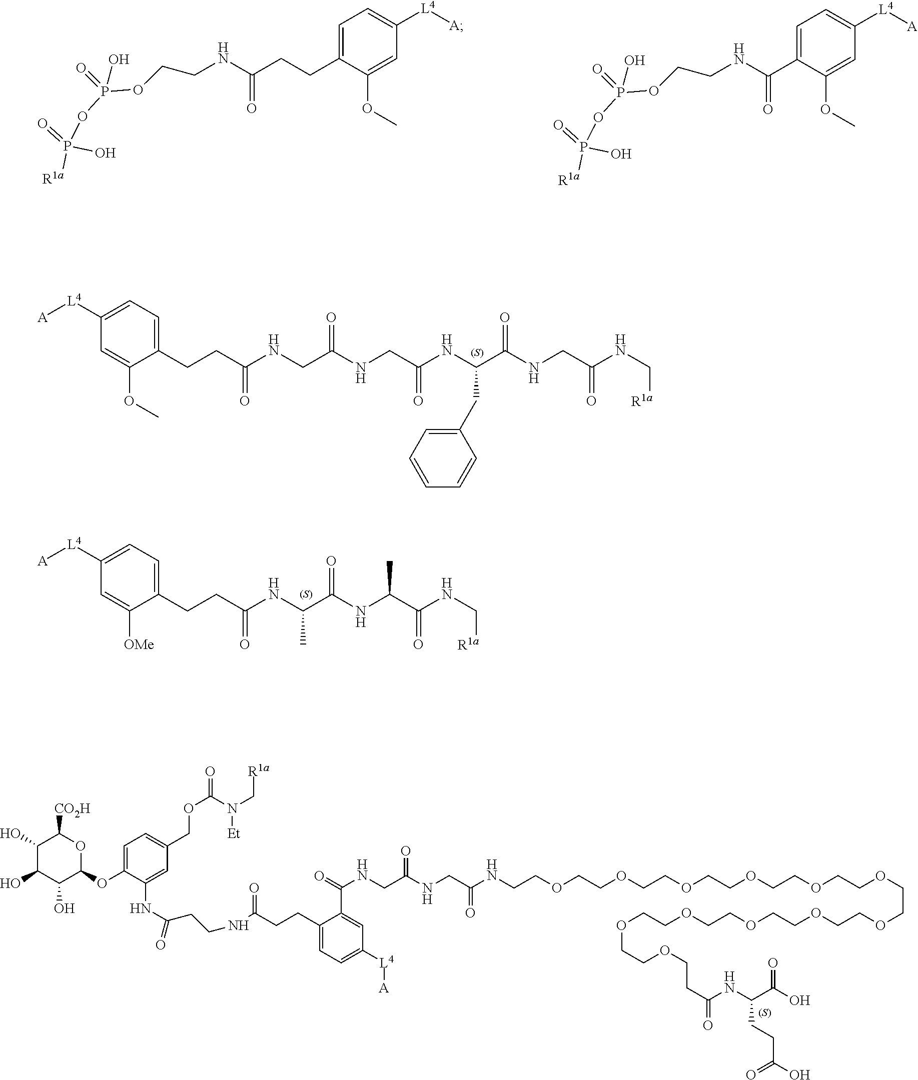

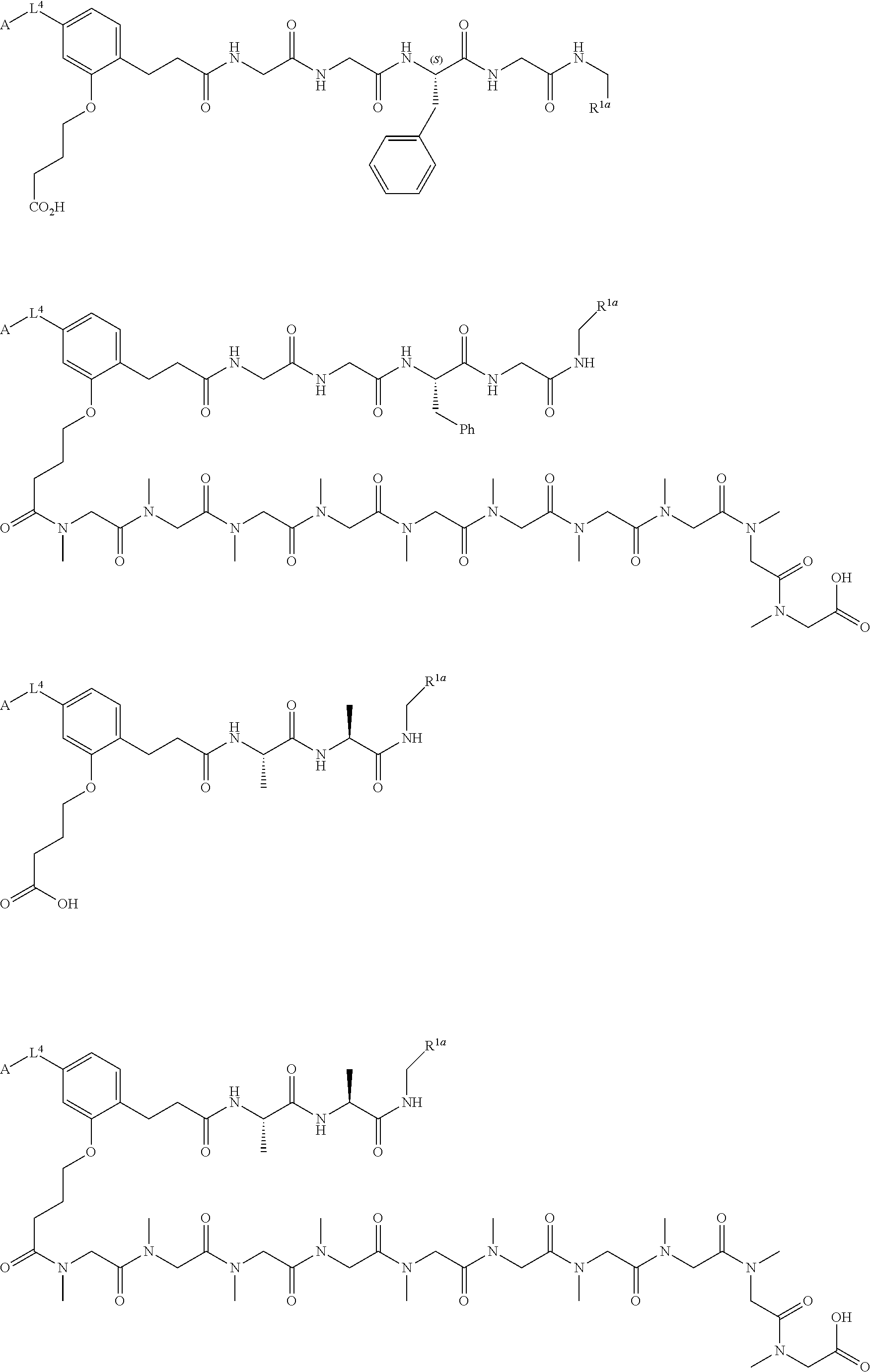

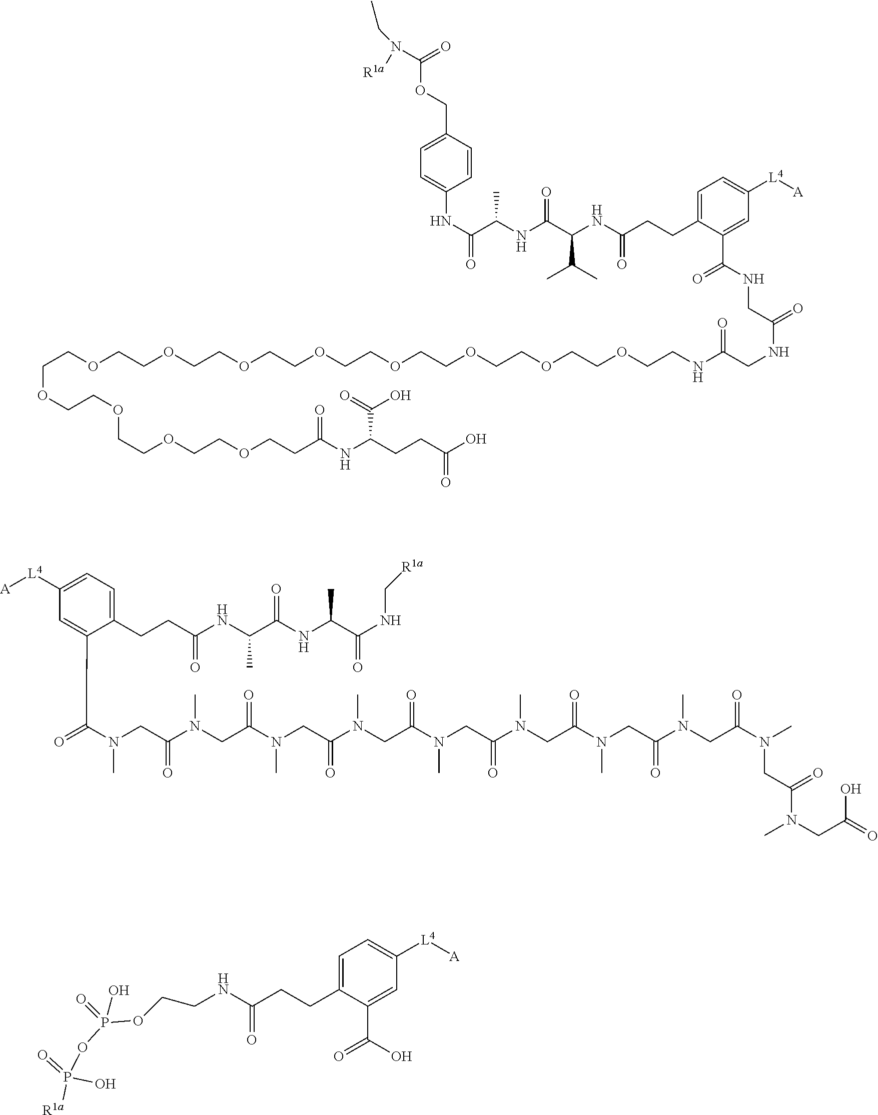

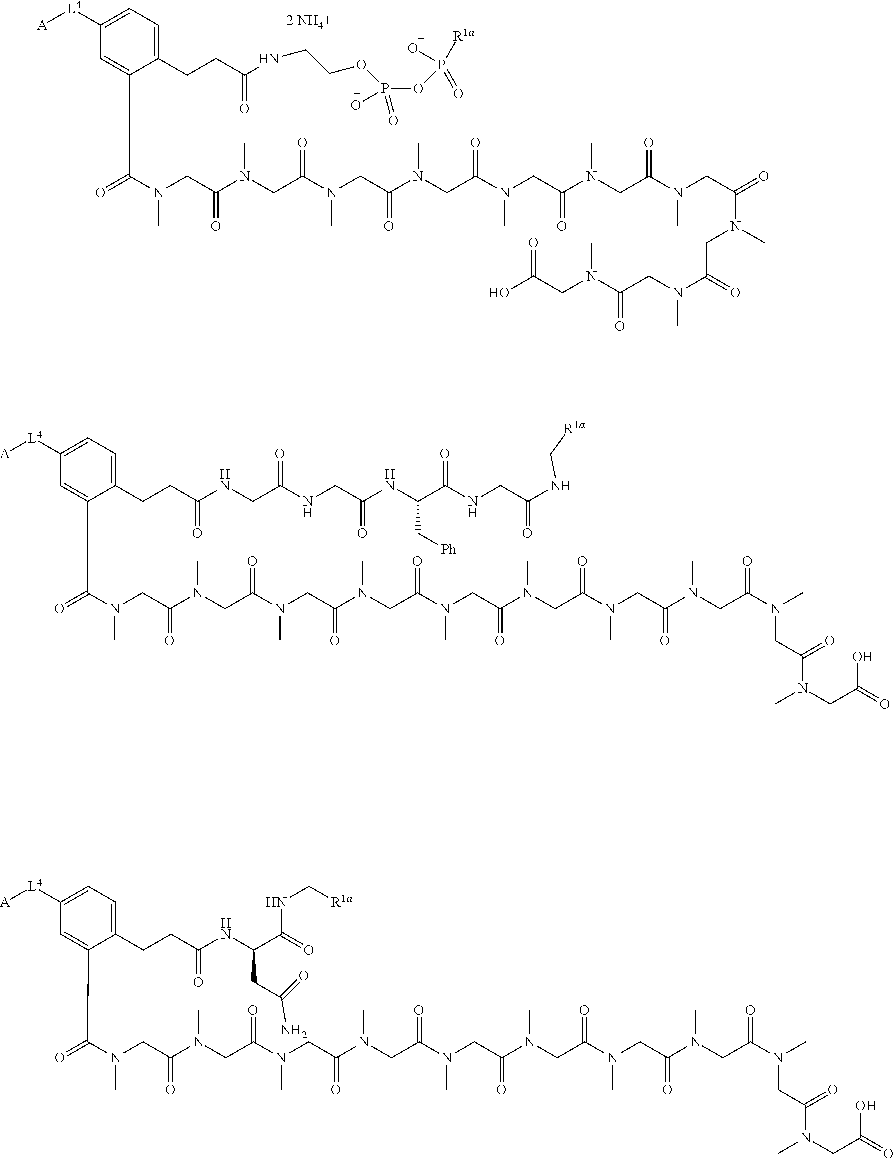

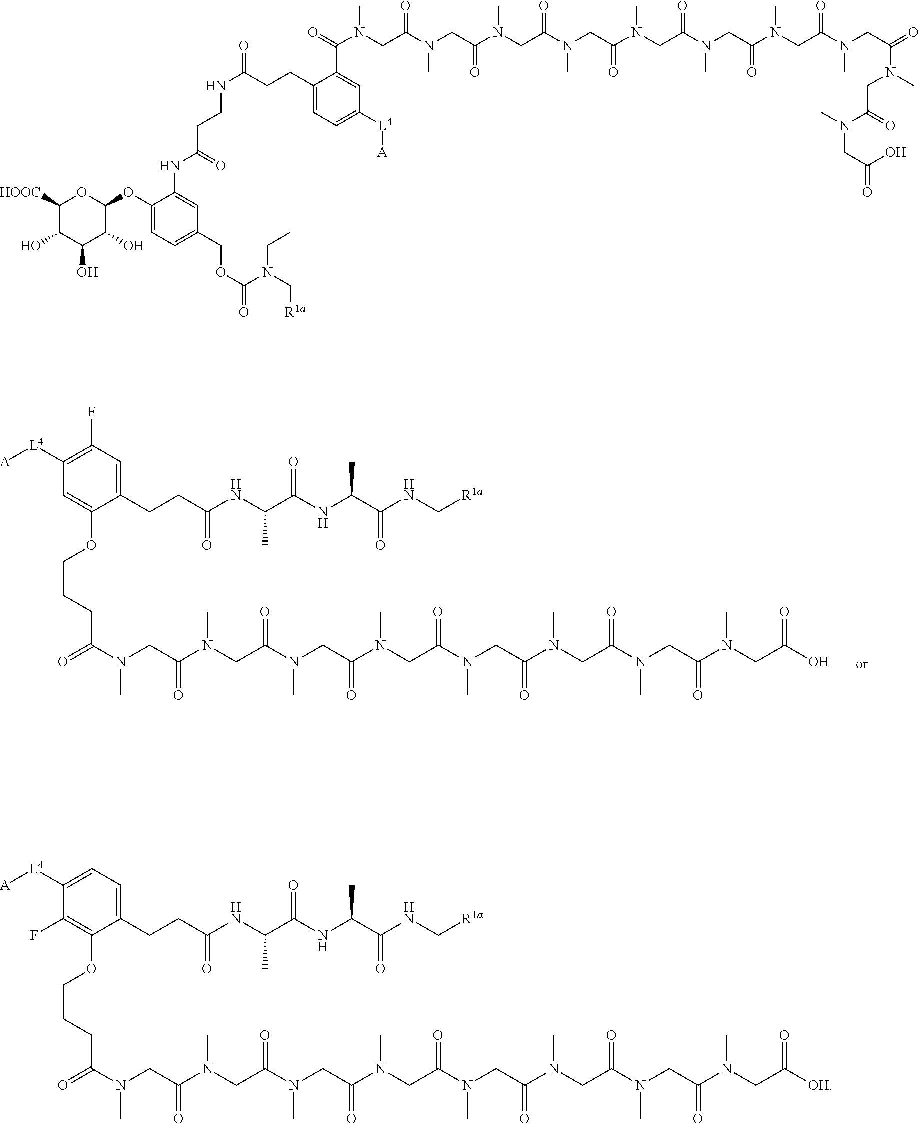

In some embodiments, a conjugate of this disclosure comprises: (a) a compound of Formula I or Formula II, or a pharmaceutically acceptable salt thereof, (b) a binding protein comprising a binding domain capable of specifically binding to a target or multiple targets, wherein the target is selected from the group consisting of CD40, CD40 Ligand, T-lymphocyte activation antigen CD86 (CD86), cytotoxic T-lymphocyte protein 4 (CTLA4), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), tumor necrosis factor receptor 2 (TNF-R2), killer cell lectin-like receptor G1 (KLRG1), B-cell-activating factor (BAFF), BAFF Receptor (BAFFR), transmembrane activator and CAML interactor (TACI), Peyer patches-specific homing receptor (LPAM-1), B-cell maturation antigen (BCMA), and a proliferation-inducing ligand (APRIL); and (c) a linker covalently attaching the compound to the binding protein.

In some embodiments, the conjugate of this disclosure comprises an anti-CD40 antibody, a compound of Formula I or Formula II or pharmaceutically acceptable salt thereof, and a Category XI linker linking the antibody to the compound. In some embodiments, the anti-CD40 antibody comprises the CDRs of bleselumab, dacetuzumab, giloralimab, iscalimab, lucatumumab, mitazalimab, ravagalimab, selicrelumab, sotigalimab, or vanalimab.

In some embodiments, the conjugate of this disclosure comprises an anti-TNFα antibody, a compound of Formula I or Formula II or pharmaceutically acceptable salt thereof, and a Category XI linker linking the antibody to the compound.

In some embodiments, the linker bound to a compound of Structure (I) or (II) (a “linker-payload”) of the present disclosure has a structure of a GR agonist-linker compound described in the Examples herein, or pharmaceutically acceptable salt thereof.

In some embodiments, the linker-payload of the present disclosure has a structure of a compound as shown in Table 7 below, or pharmaceutically acceptable salt thereof.

TABLE 7
Linker-Payloads
Structure
Compound Number

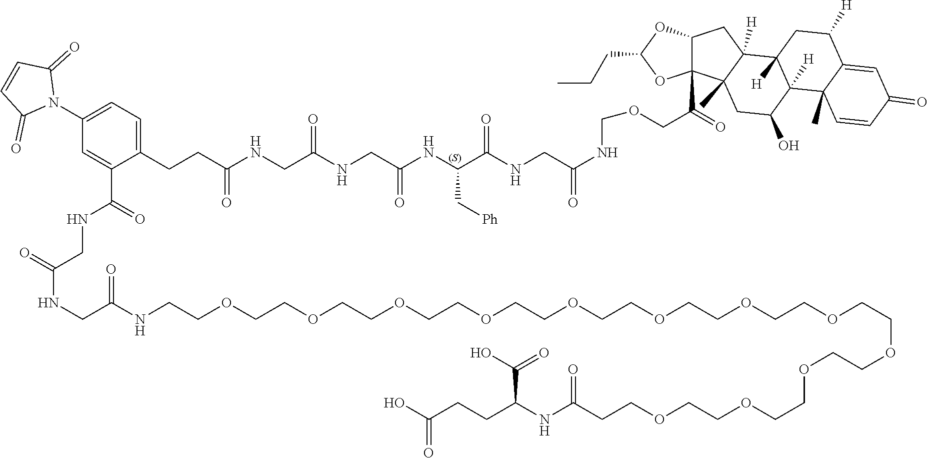

II-1

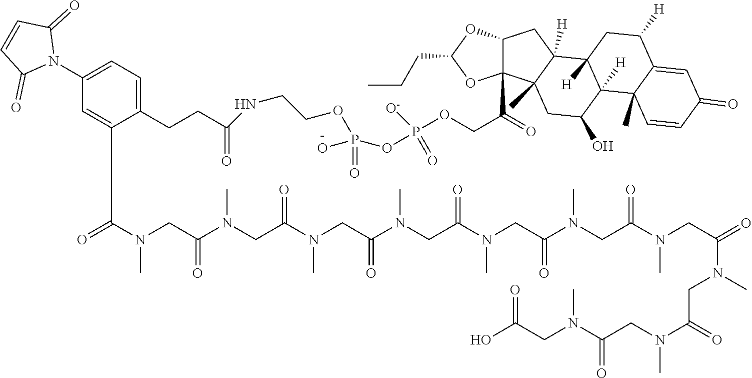

II-2

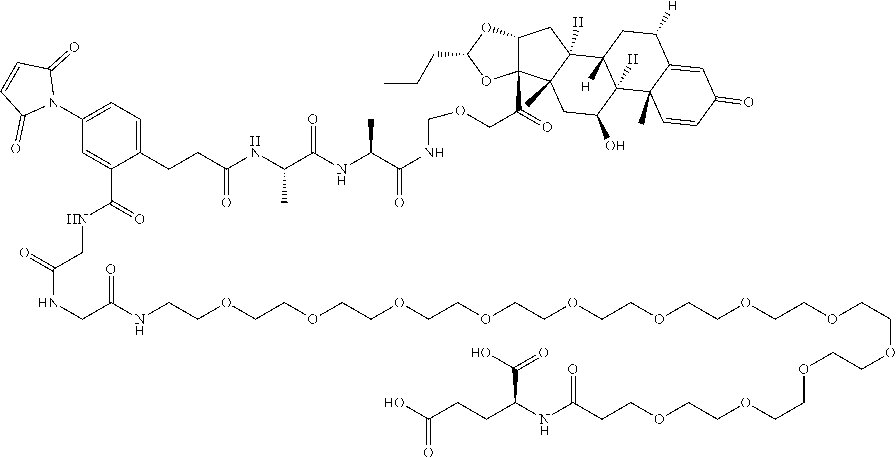

II-3

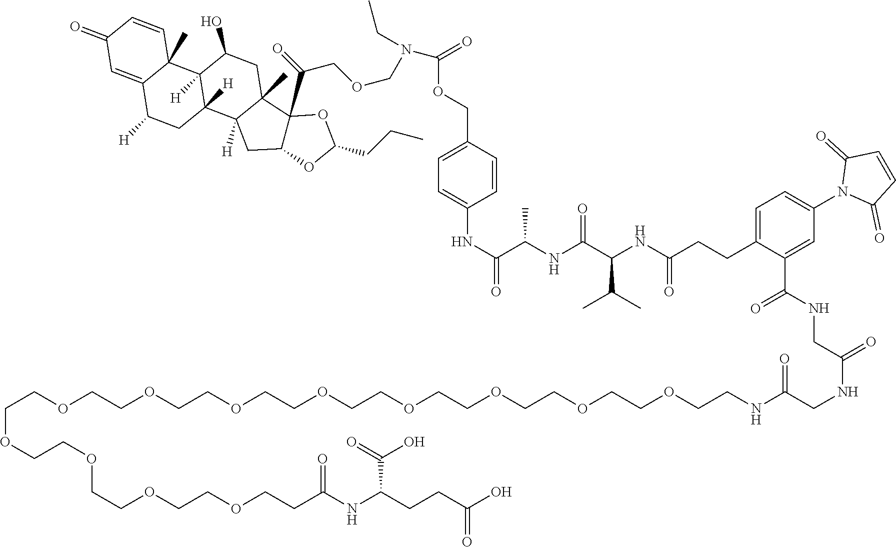

II-4

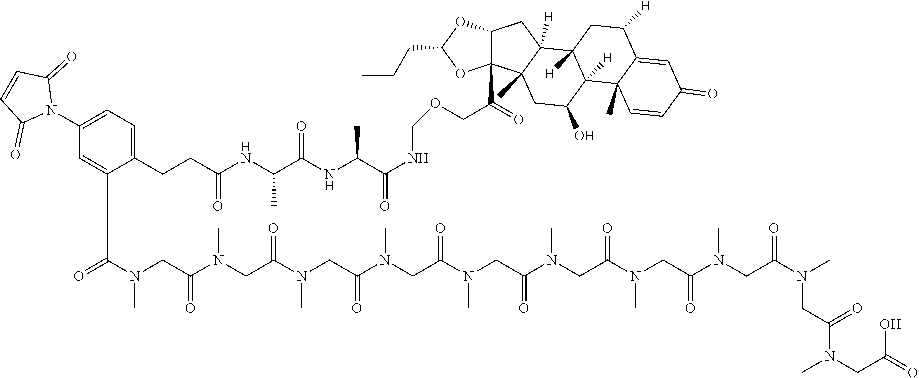

II-5

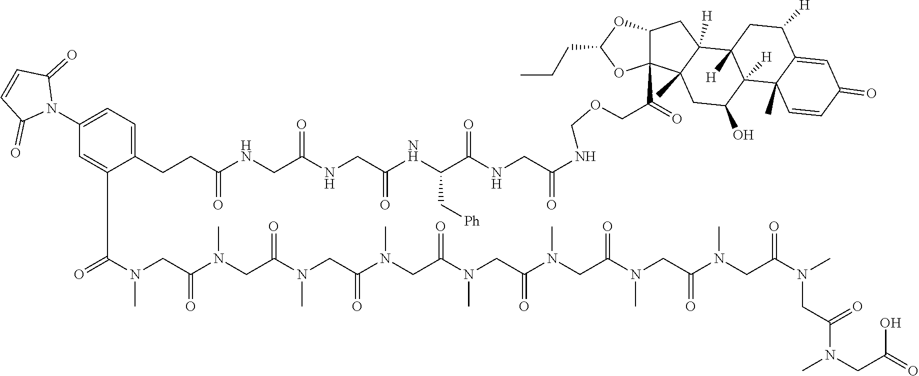

II-6

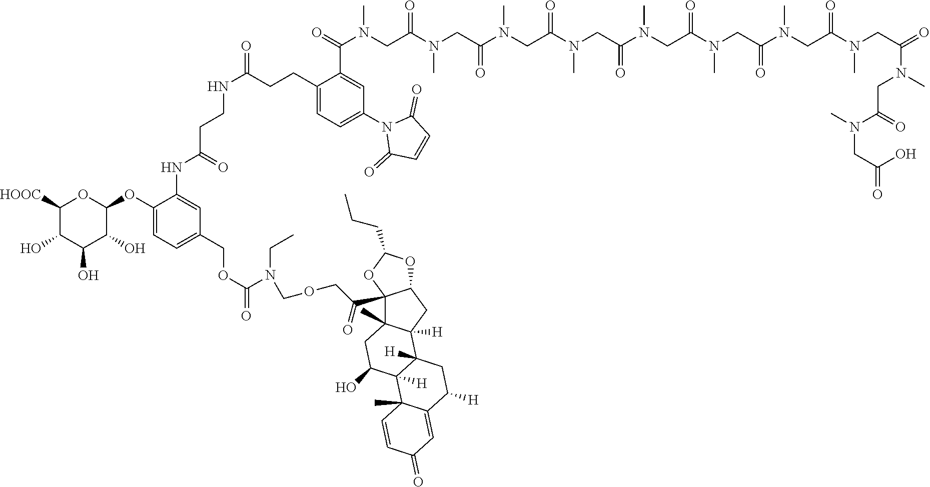

II-7

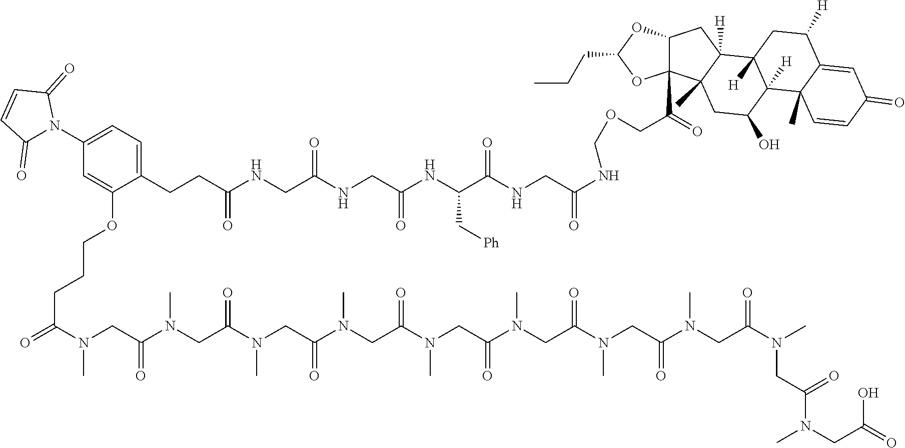

II-8

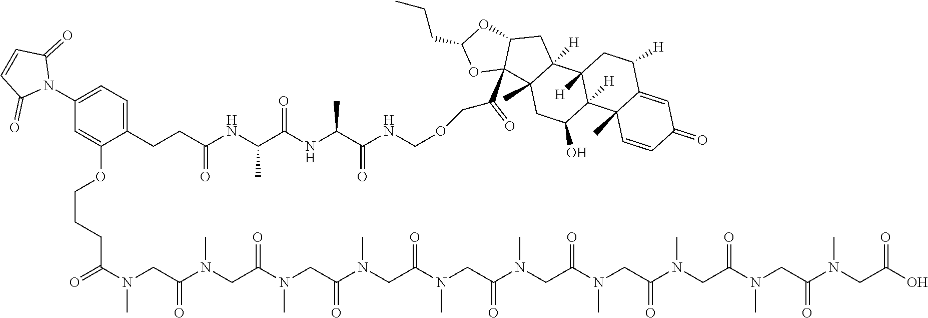

II-9

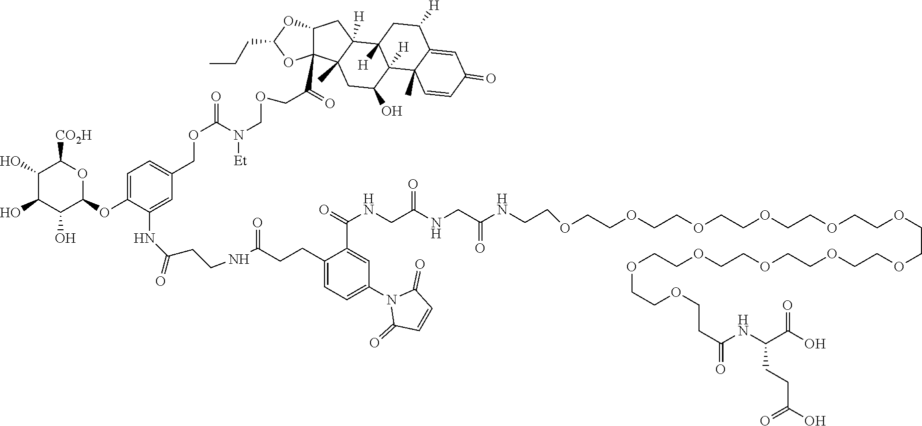

II-10

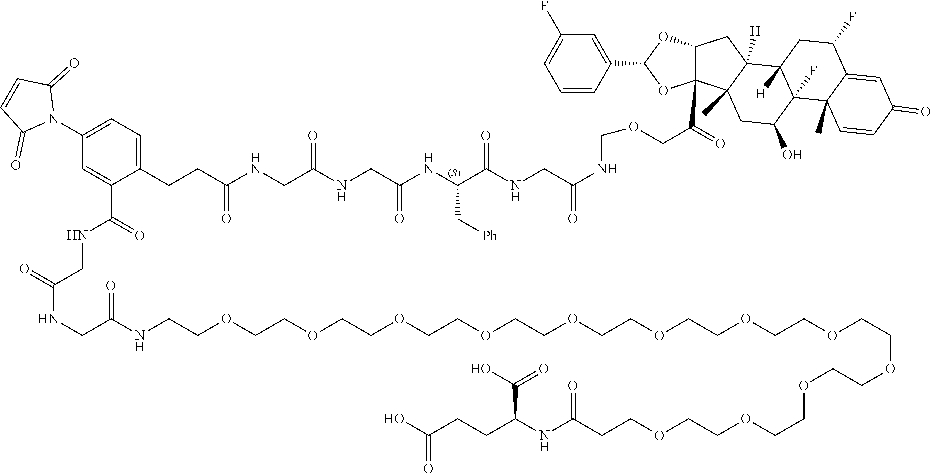

II-11

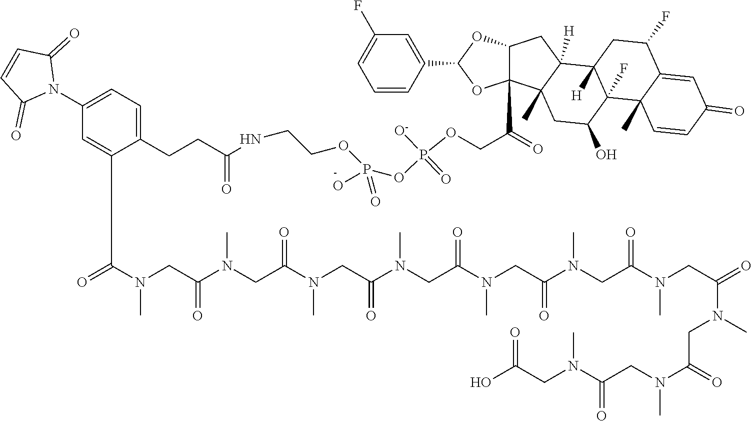

II-12

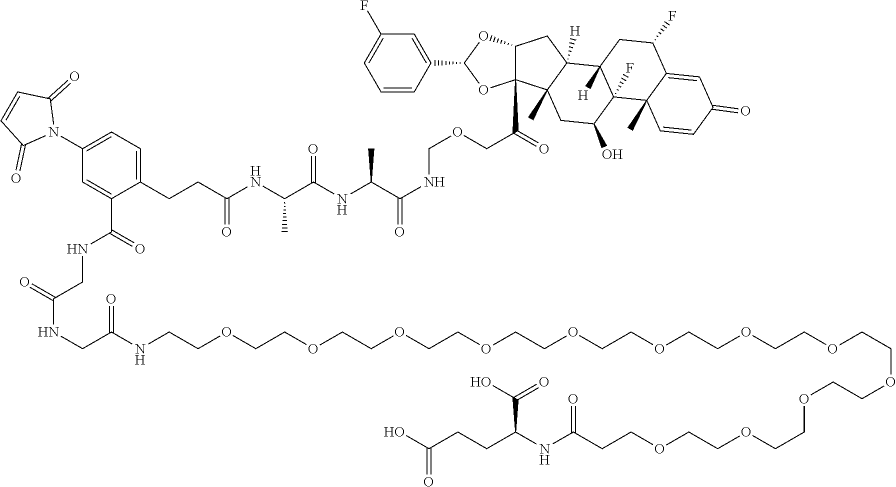

II-13

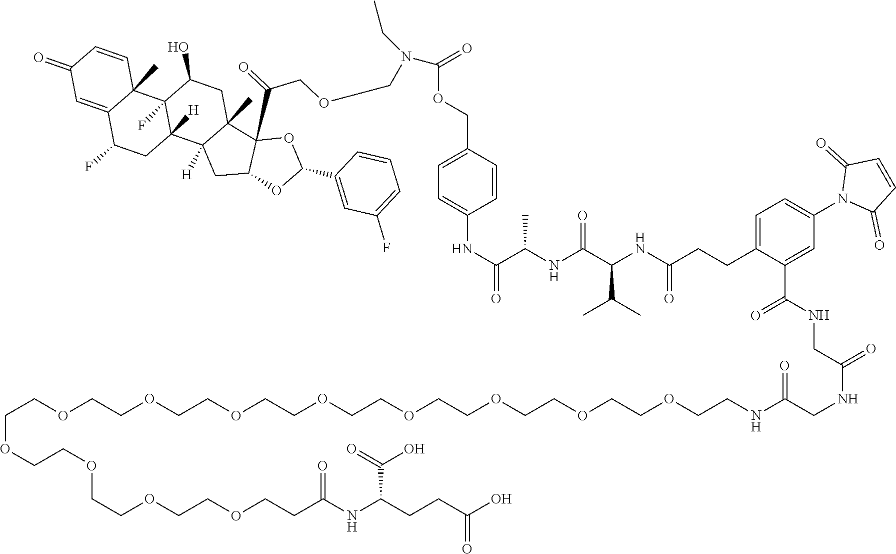

II-14

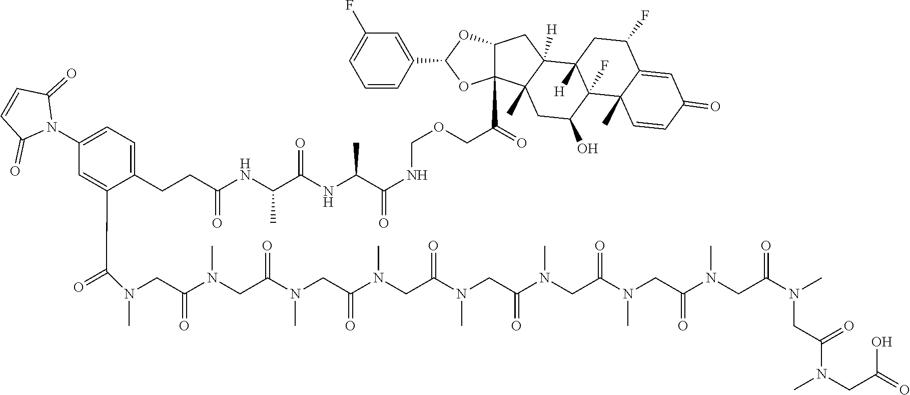

II-15

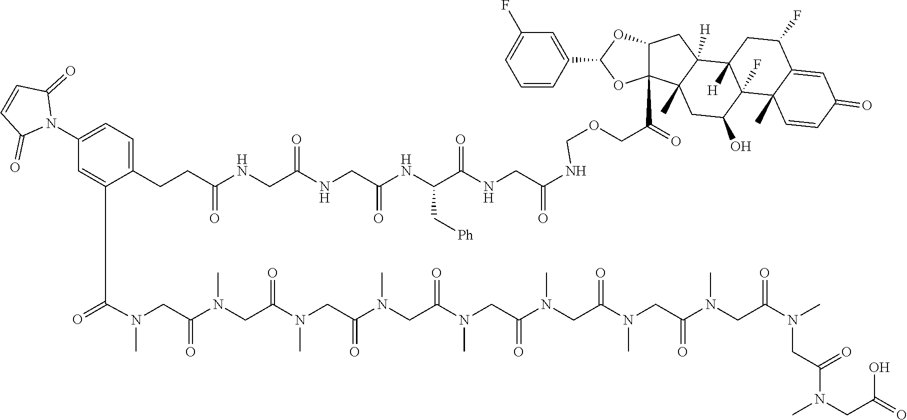

II-16

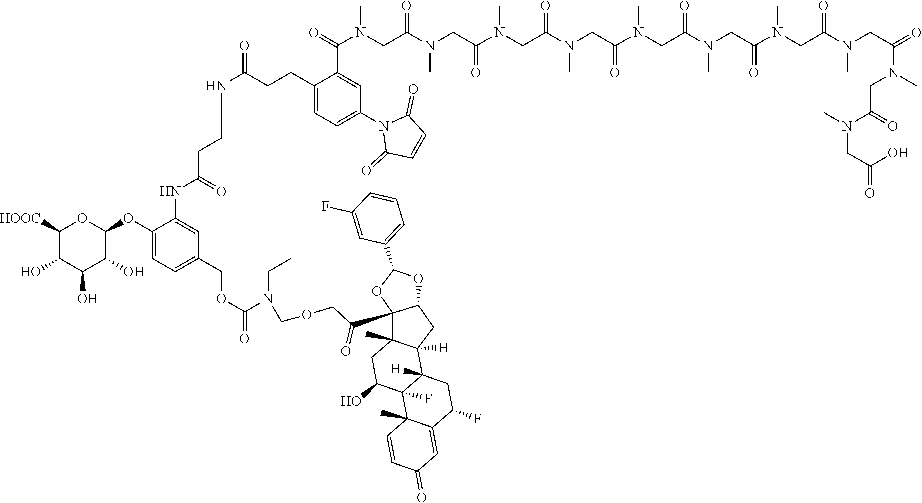

II-17

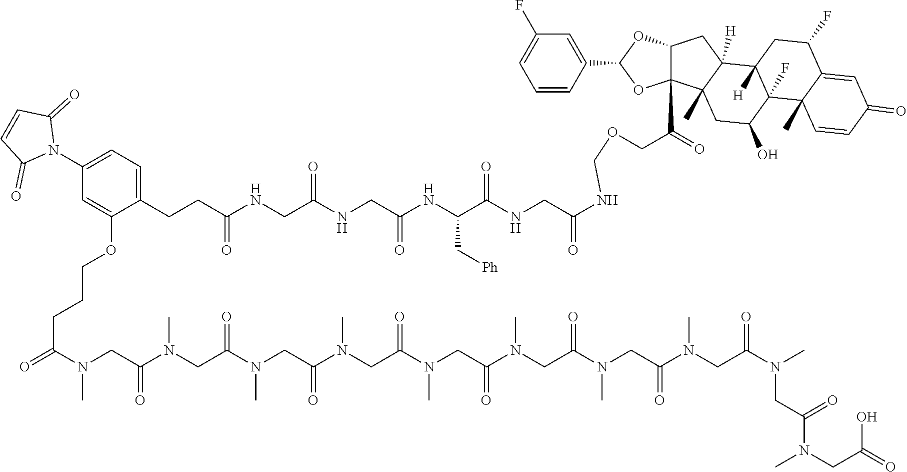

II-18

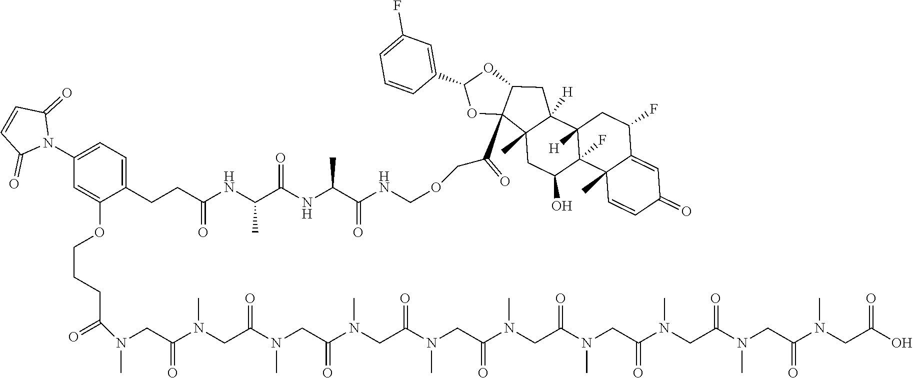

II-19

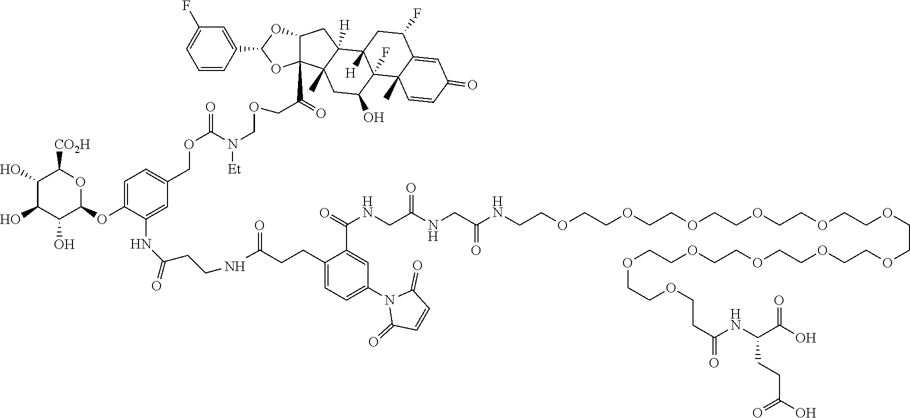

II-20

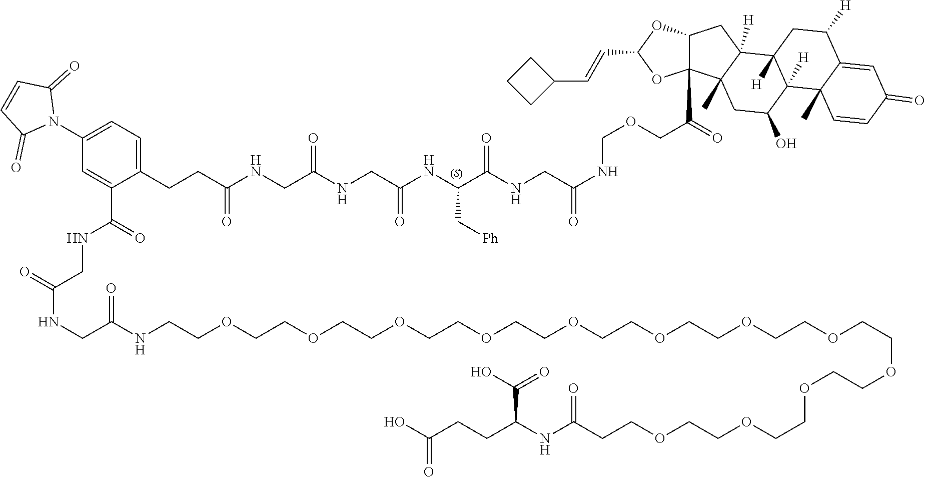

II-21

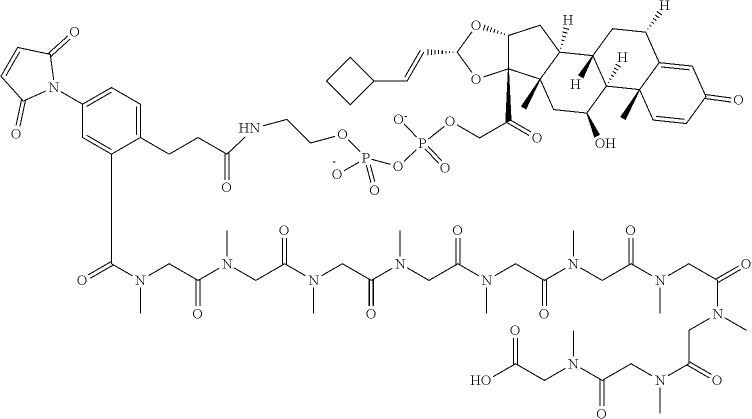

II-22

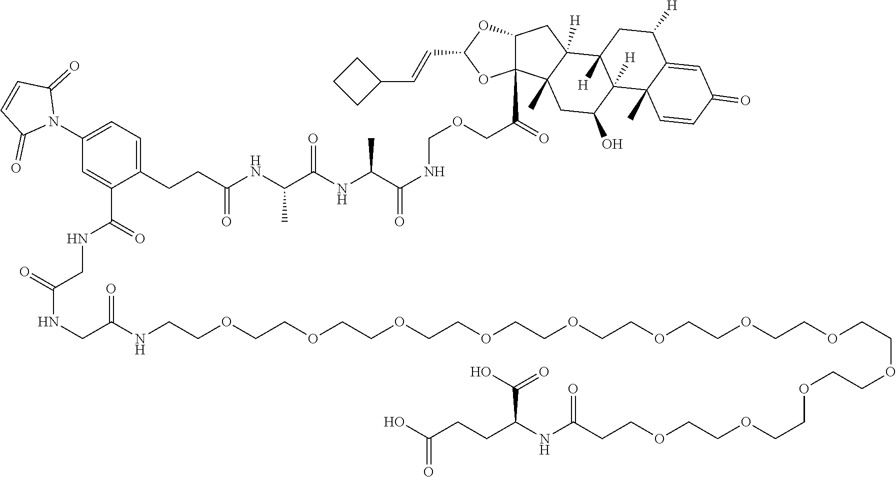

II-23

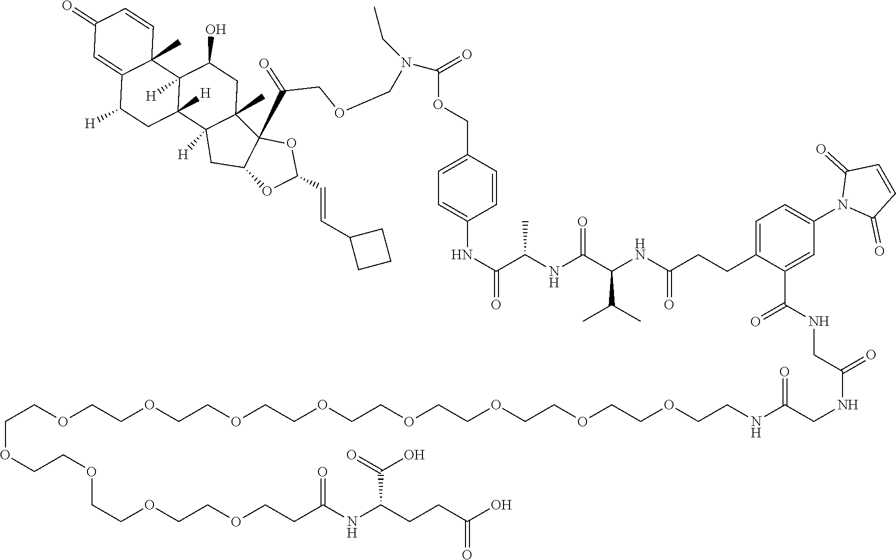

II-24

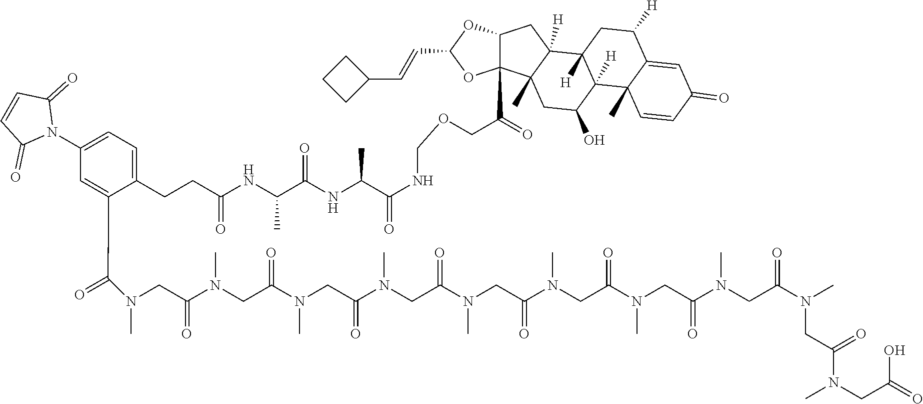

II-25

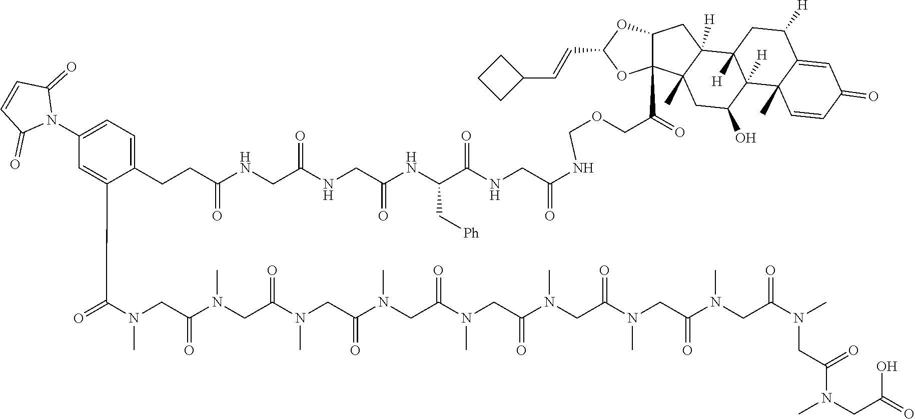

II-26

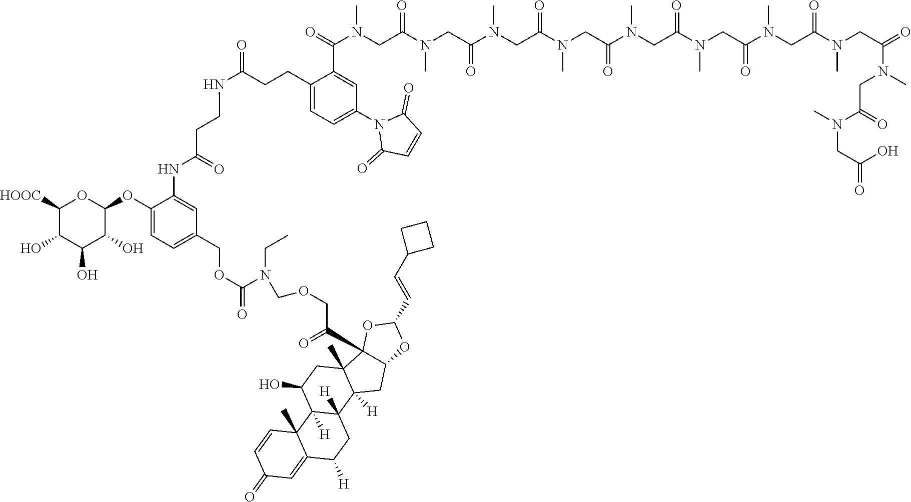

II-27

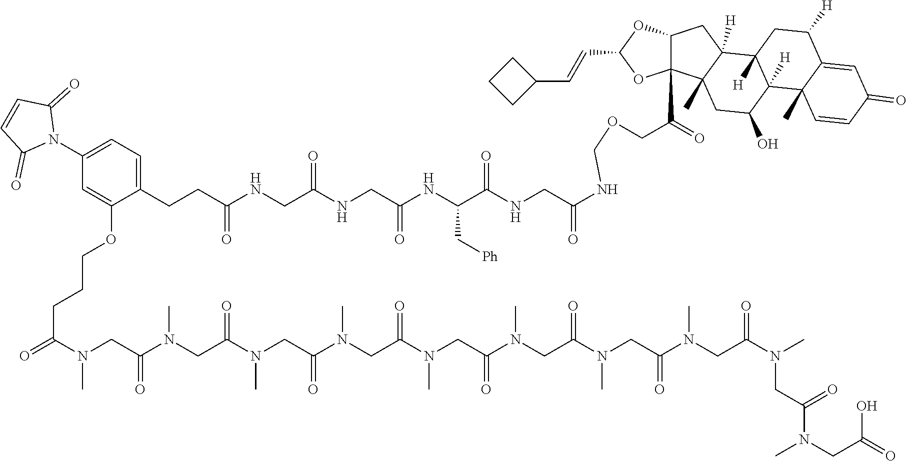

II-28

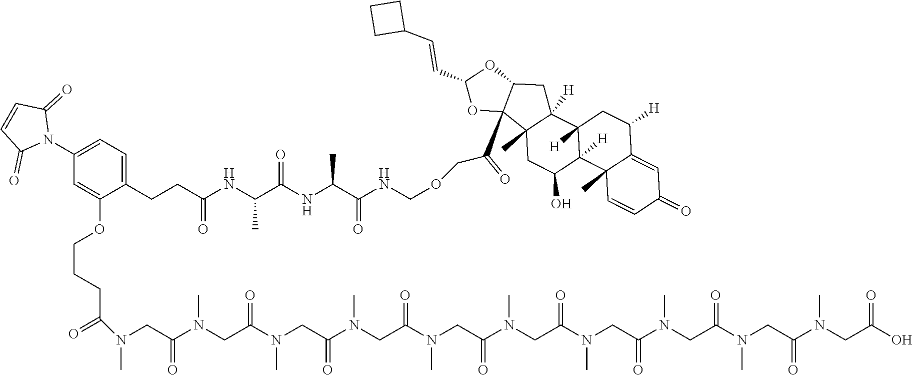

II-29

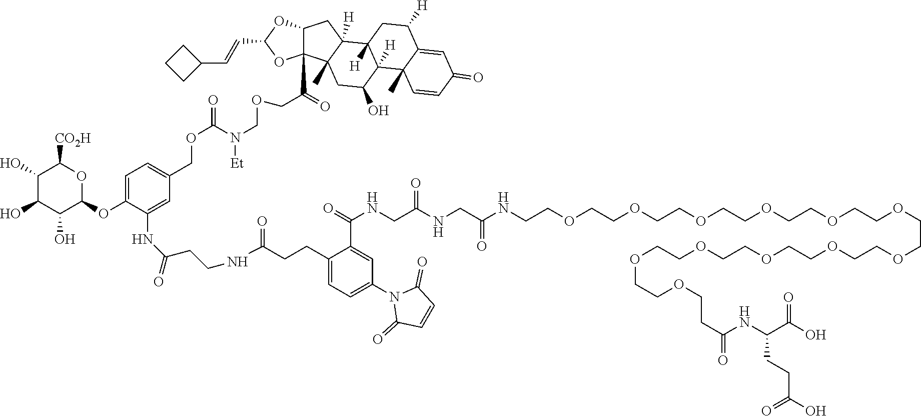

II-30

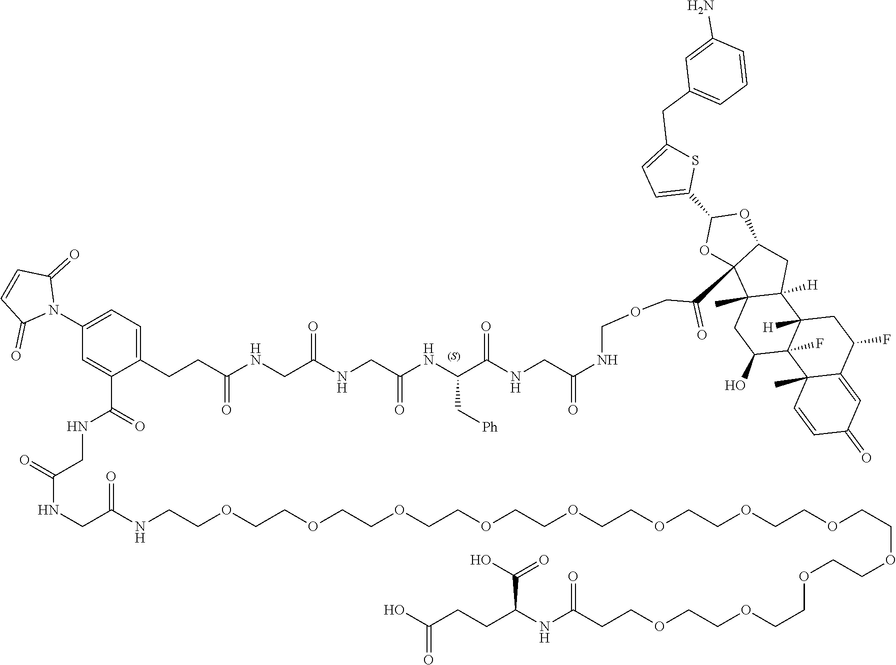

II-31

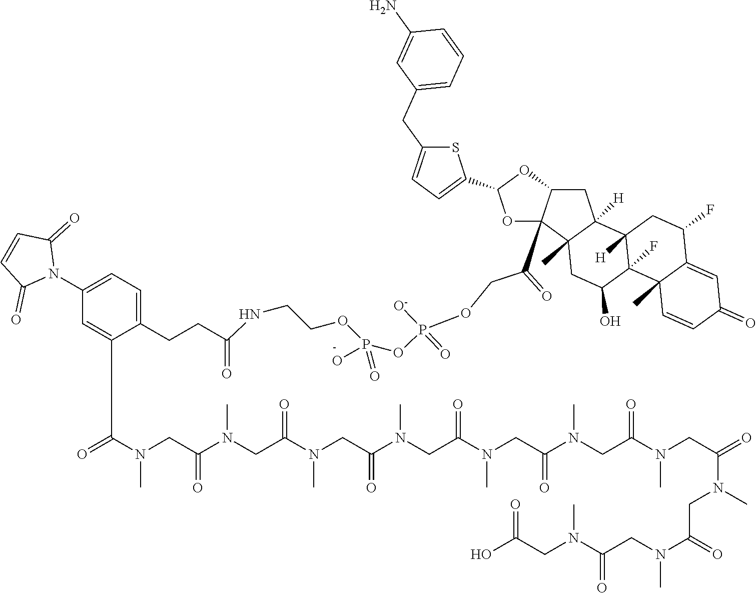

II-32

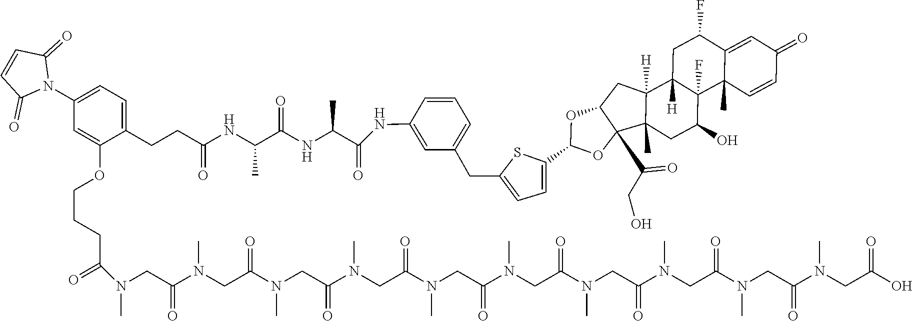

II-33

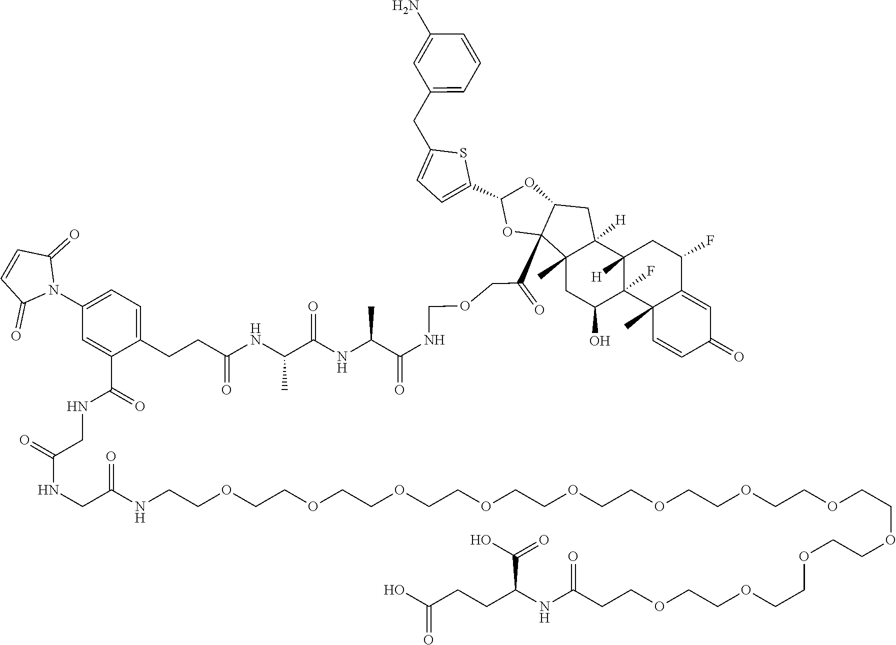

II-34

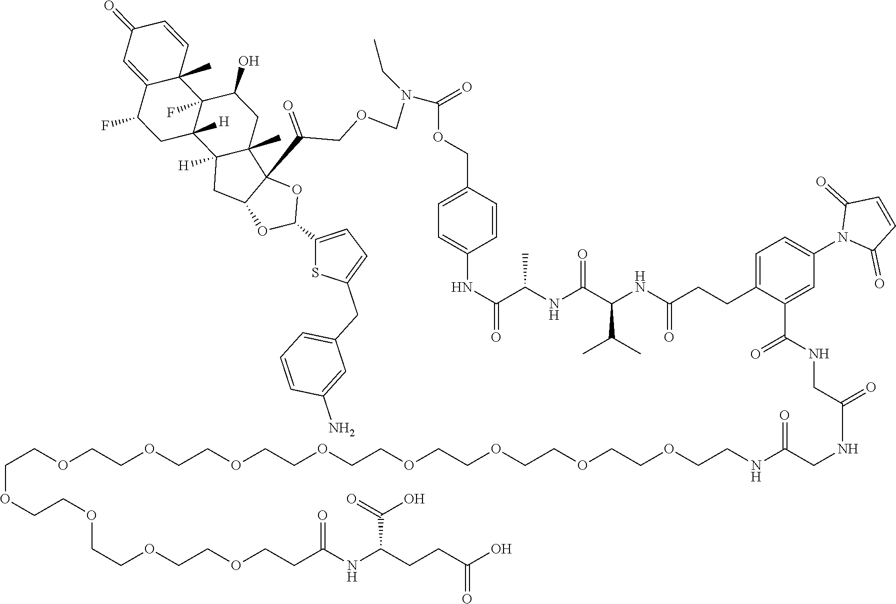

II-35

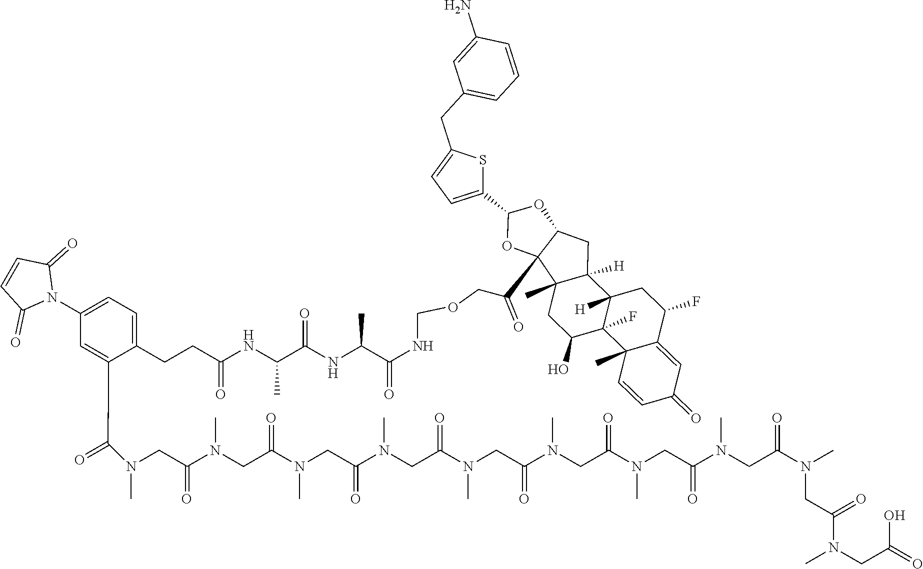

II-36

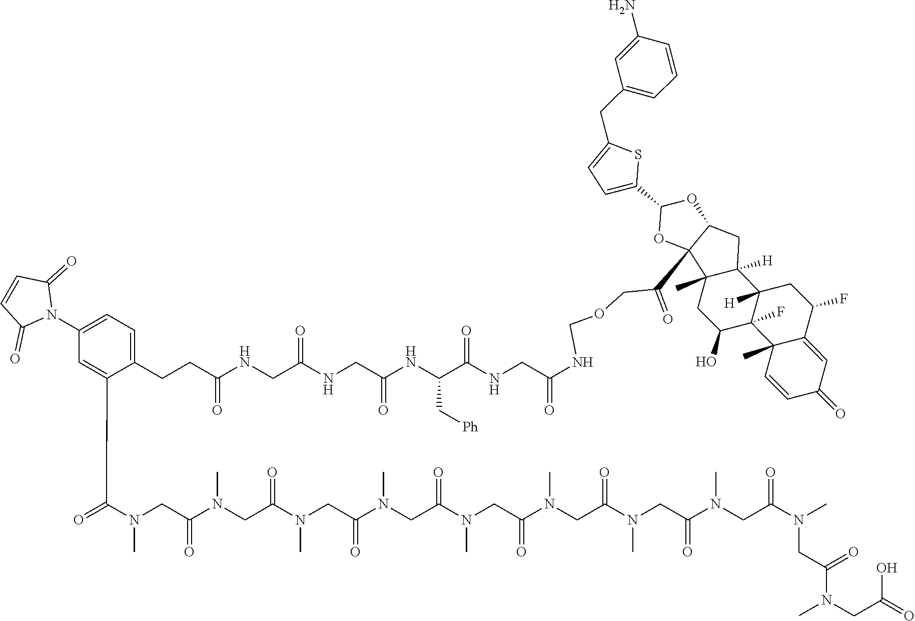

II-37

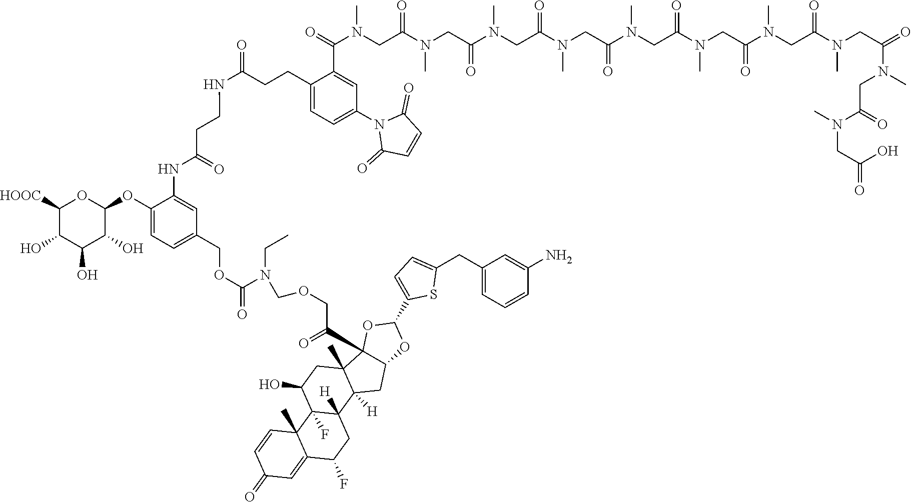

II-38

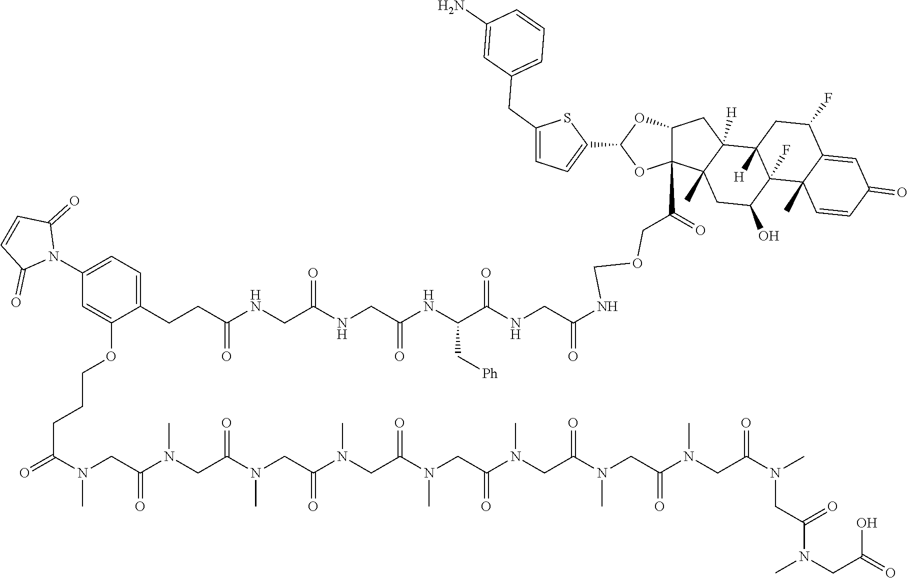

II-39

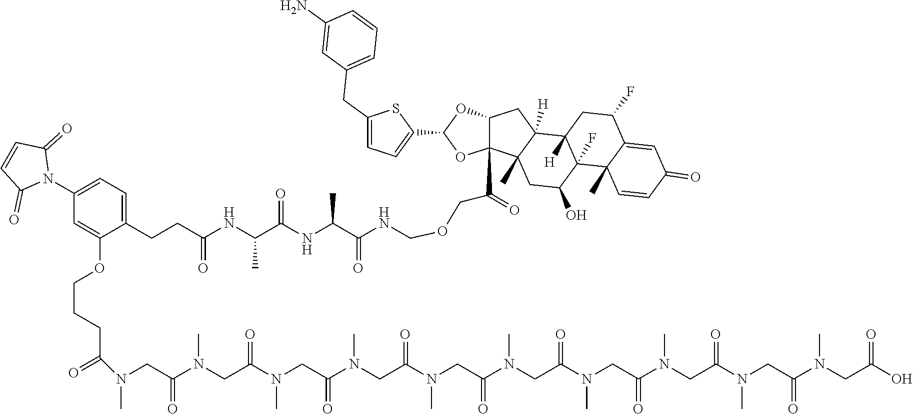

II-40

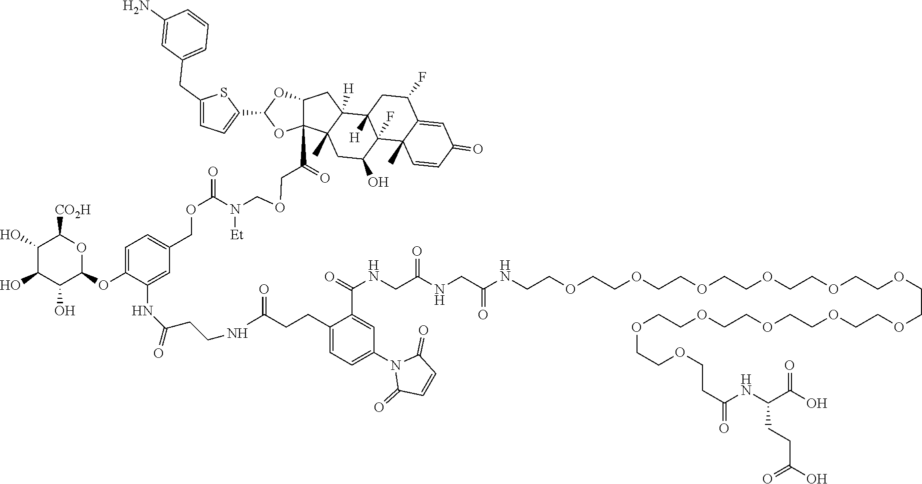

II-41

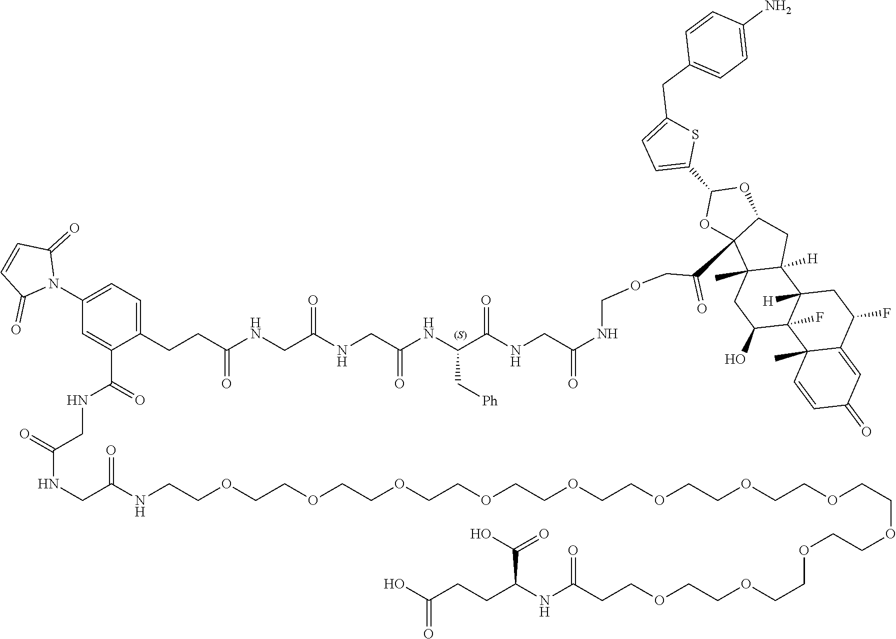

II-42

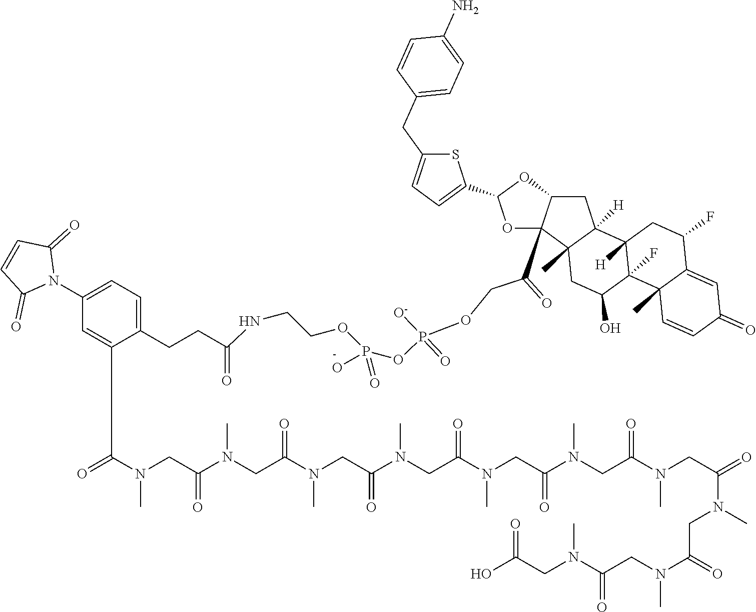

II-43

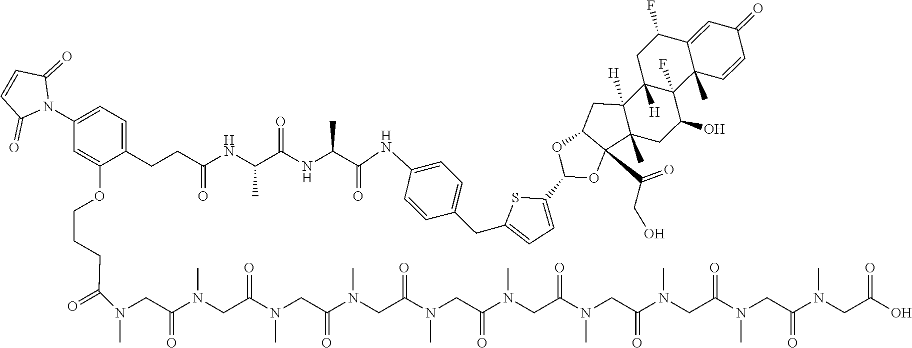

II-44

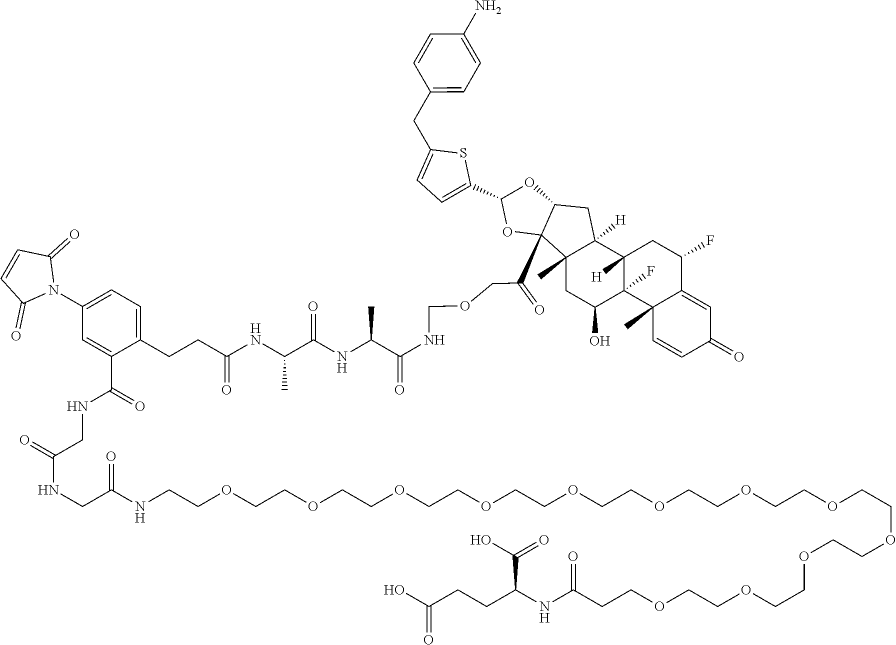

II-45

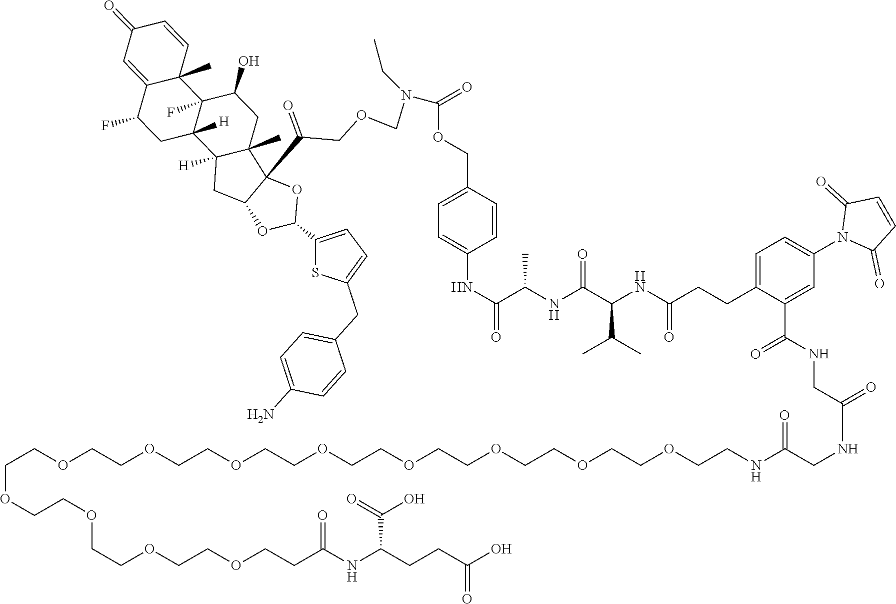

II-46

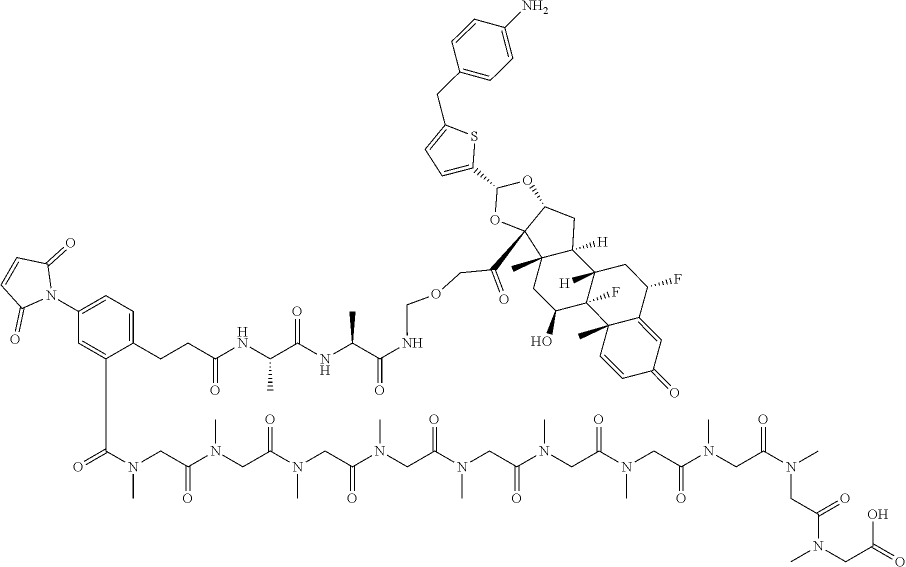

II-47

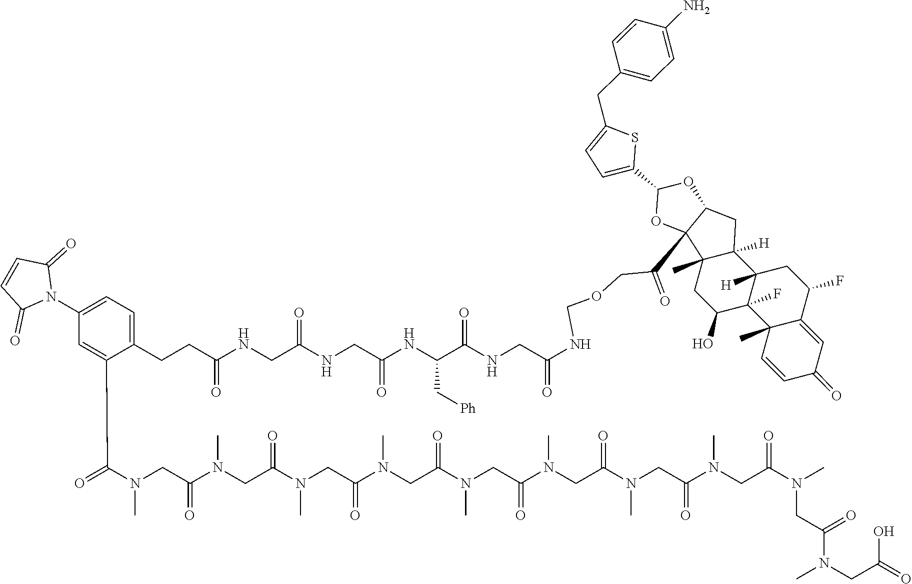

II-48

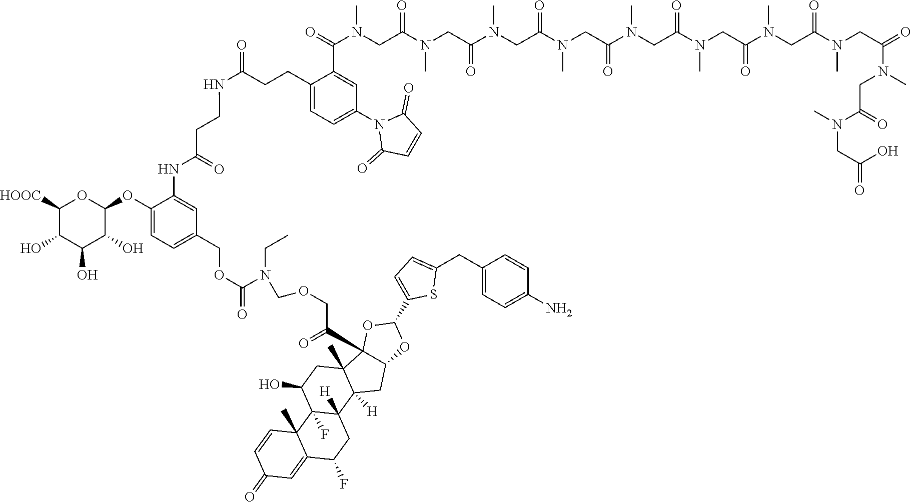

II-49

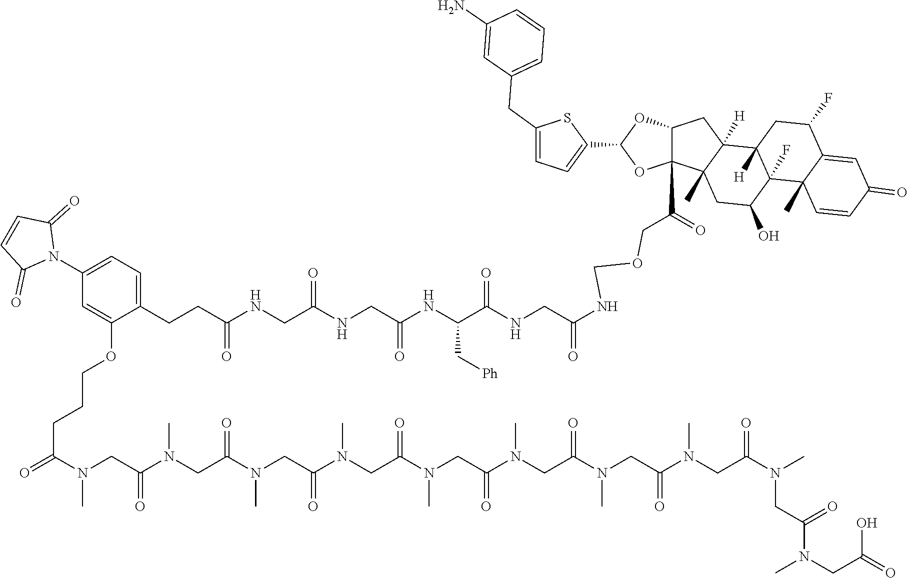

II-50

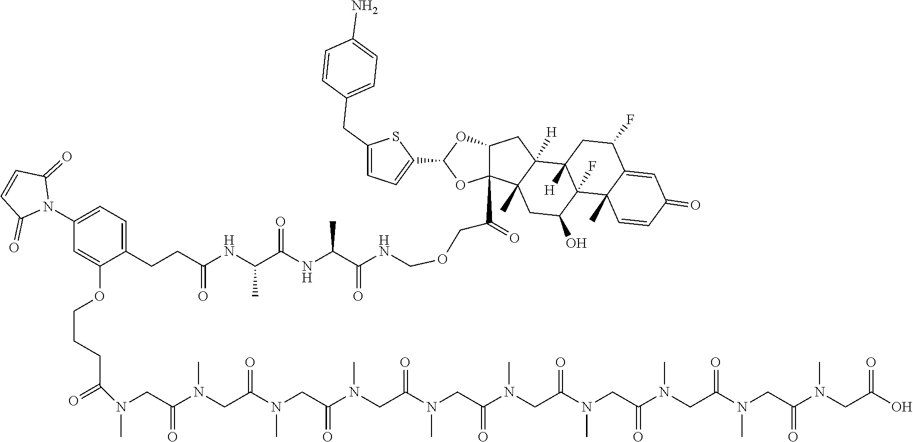

II-51

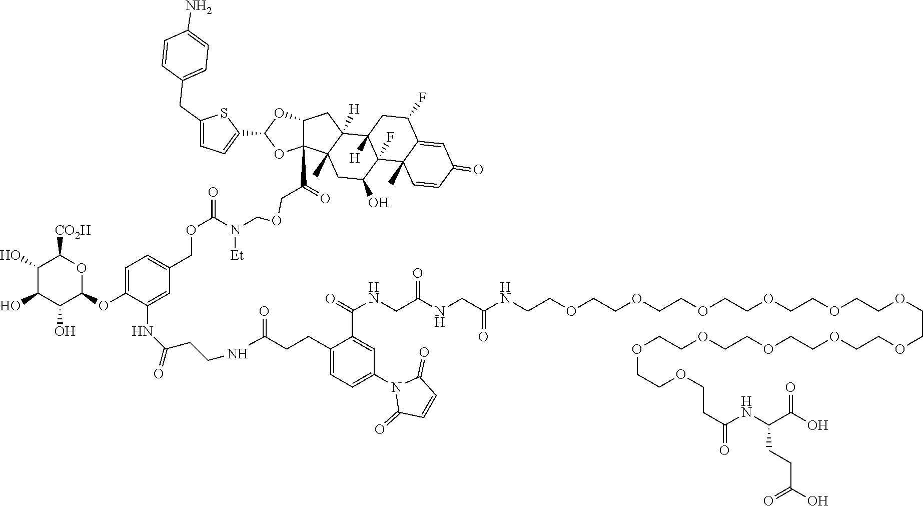

II-52

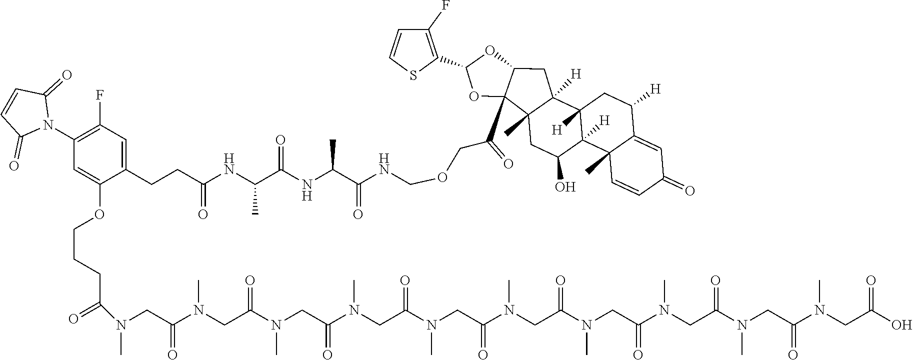

II-53

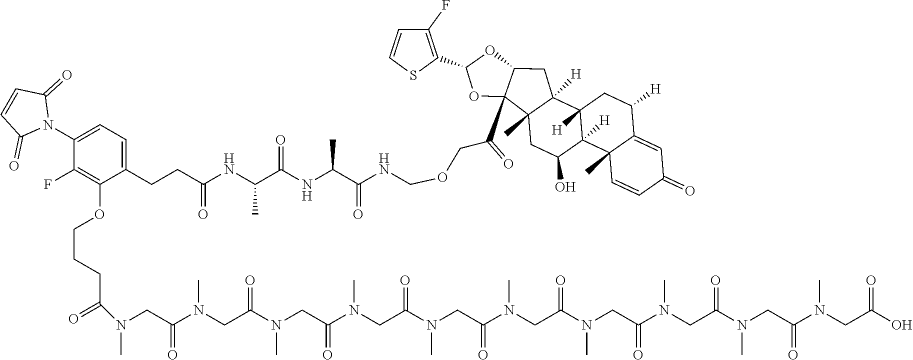

II-54

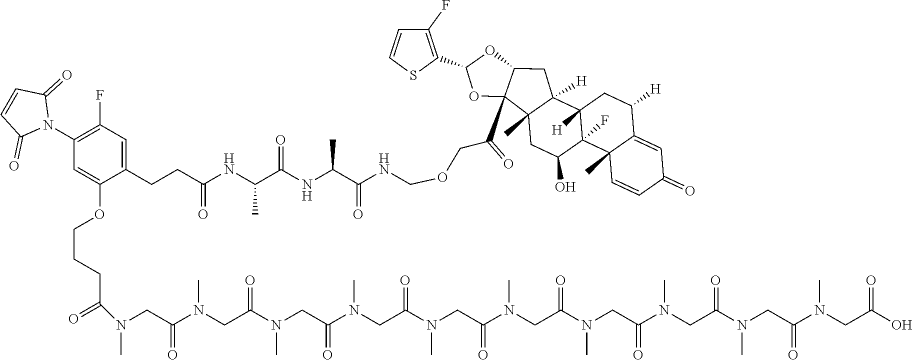

II-55

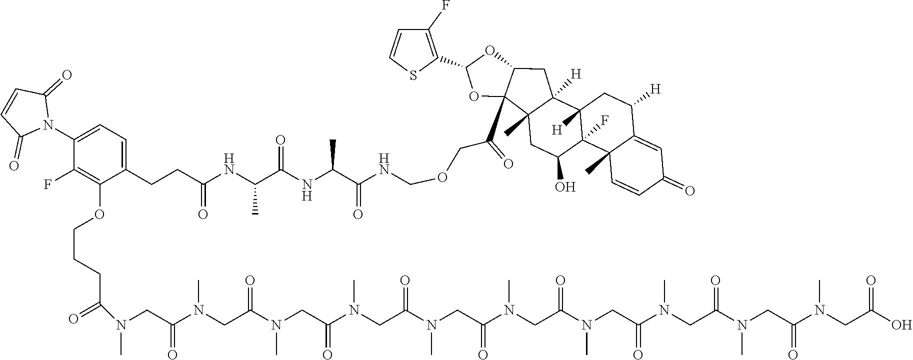

II-56

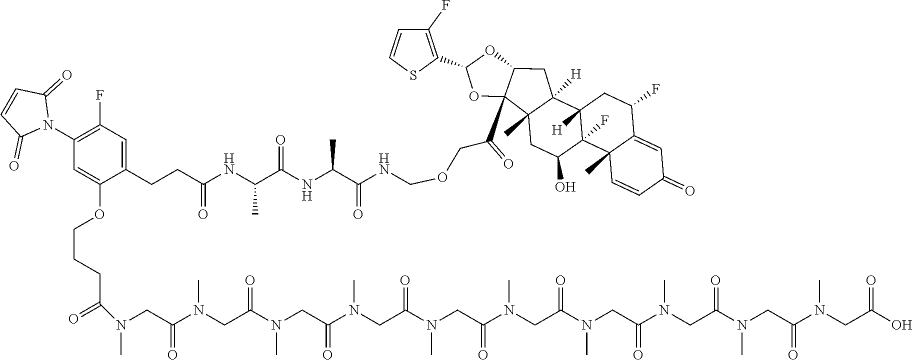

II-57

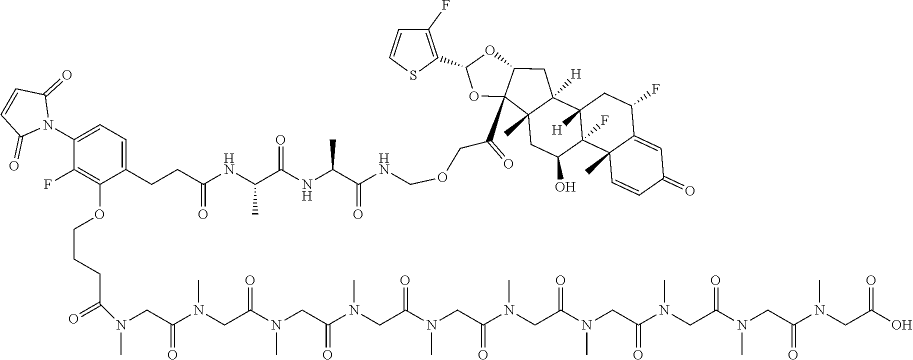

II-58

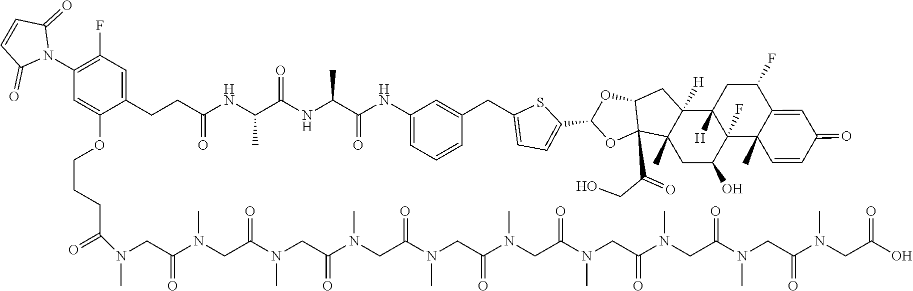

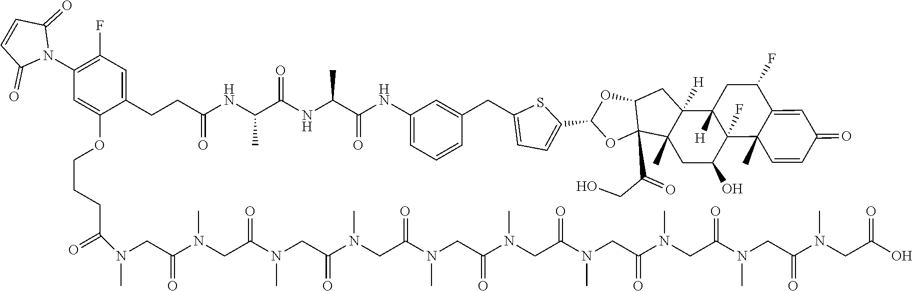

II-60

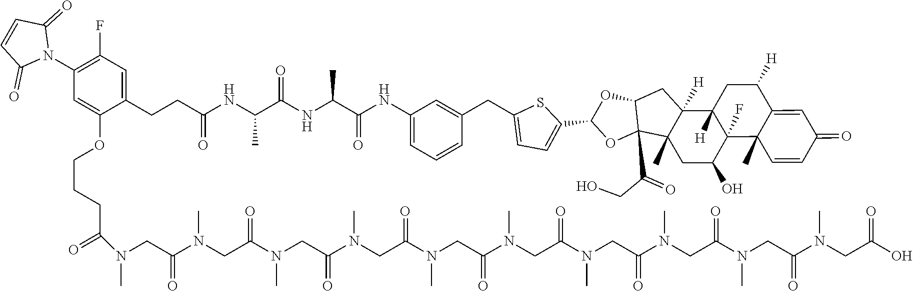

II-61

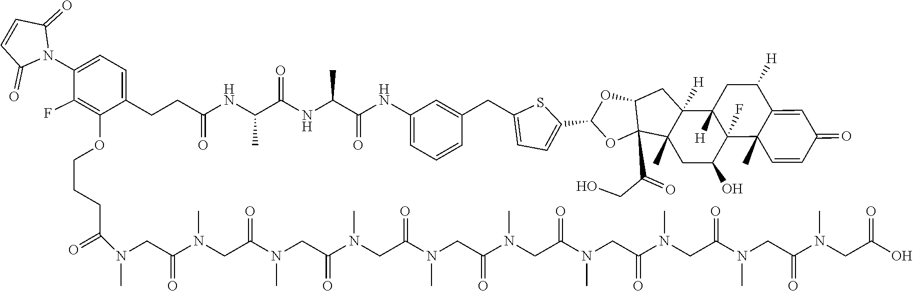

II-62

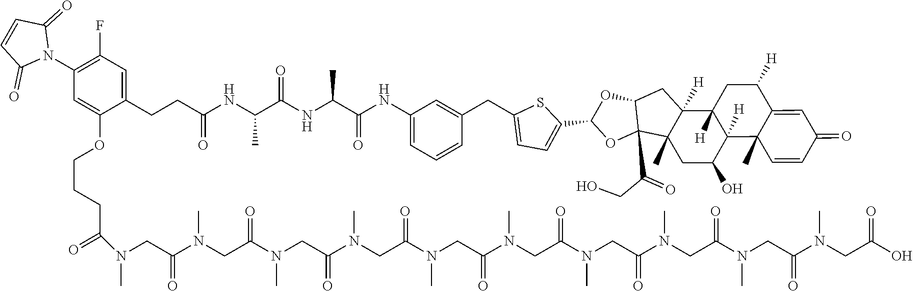

II-63

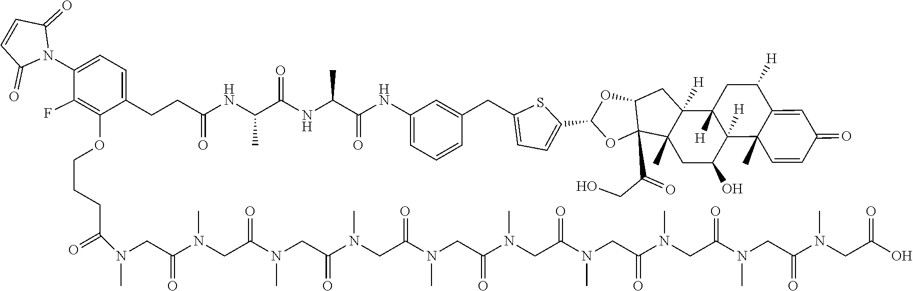

II-64

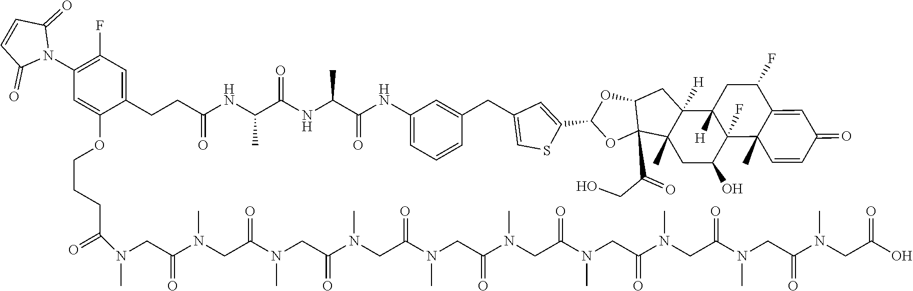

II-65

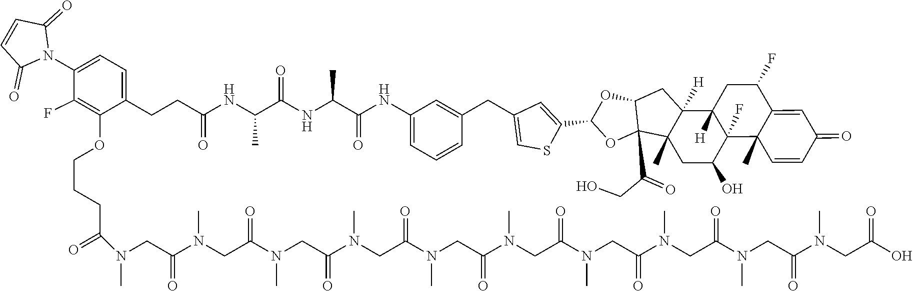

II-66

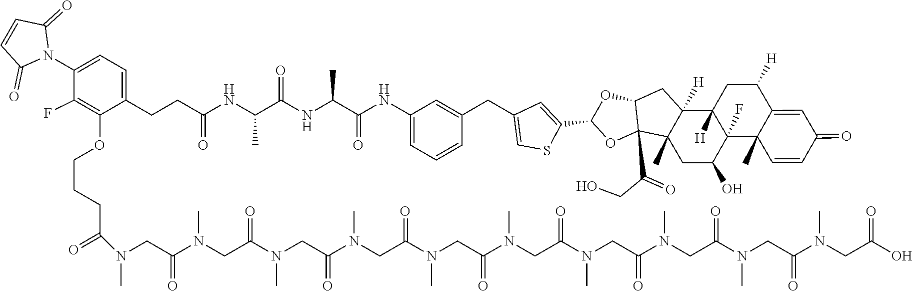

II-67

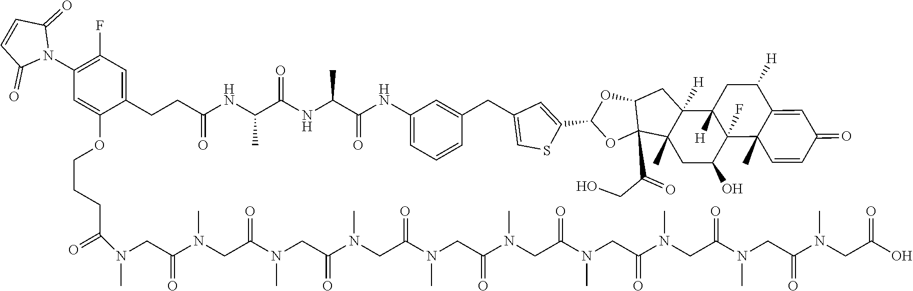

II-68

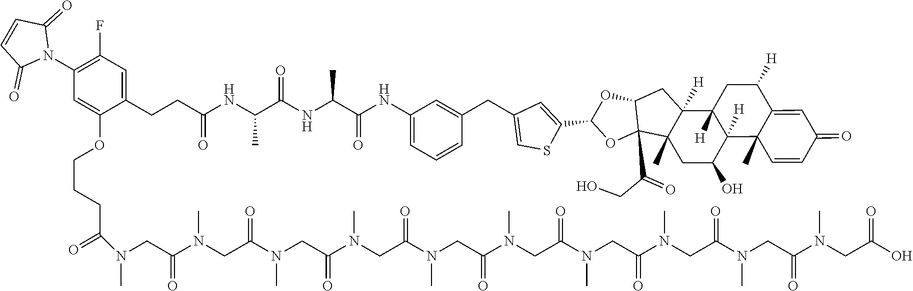

II-69

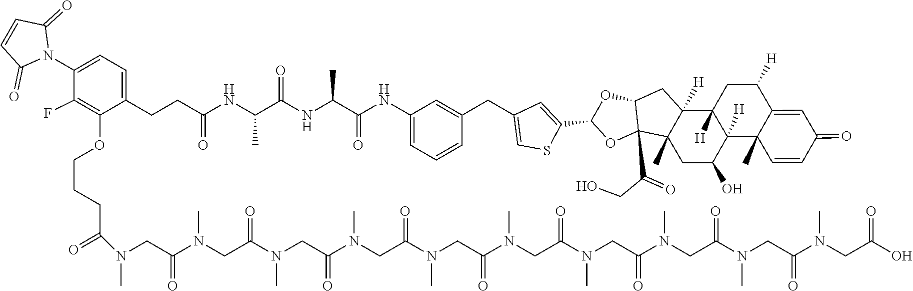

II-70

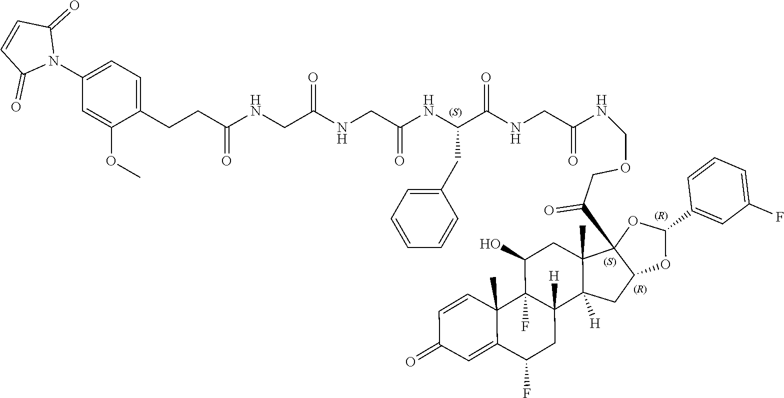

II-71

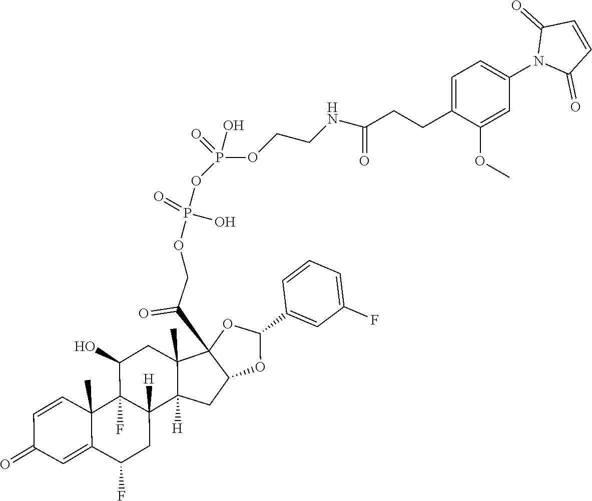

II-72

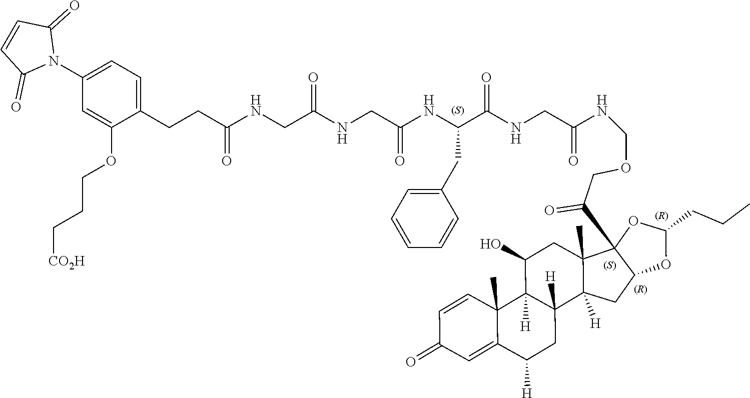

II-73

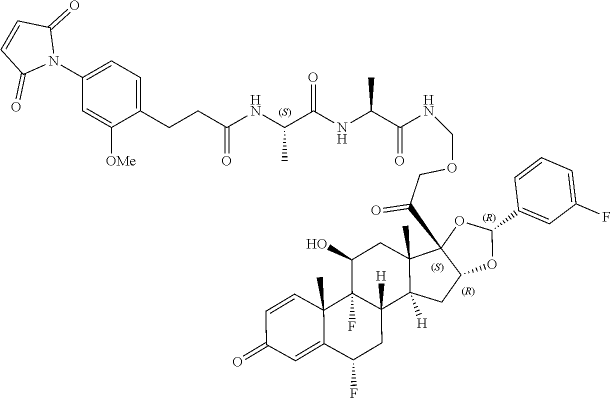

II-74

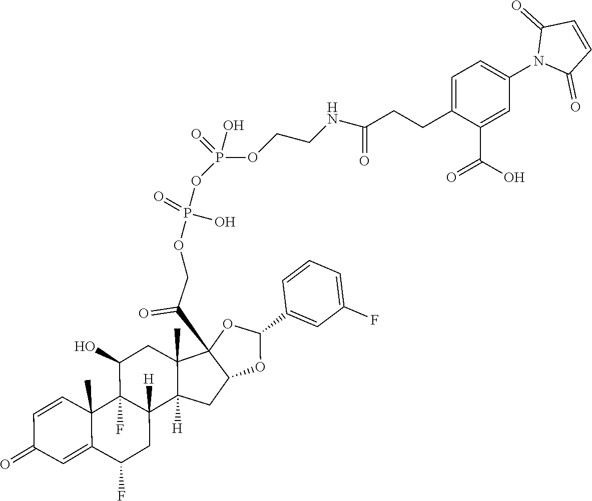

II-75

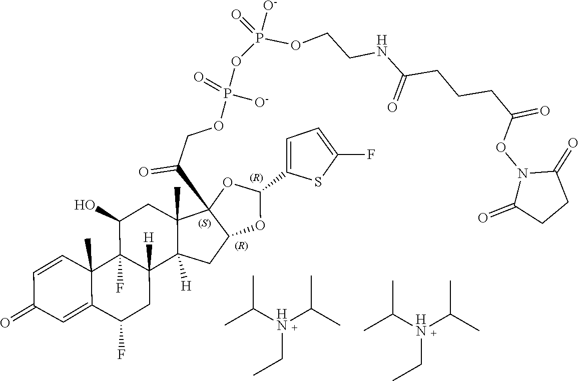

II-76

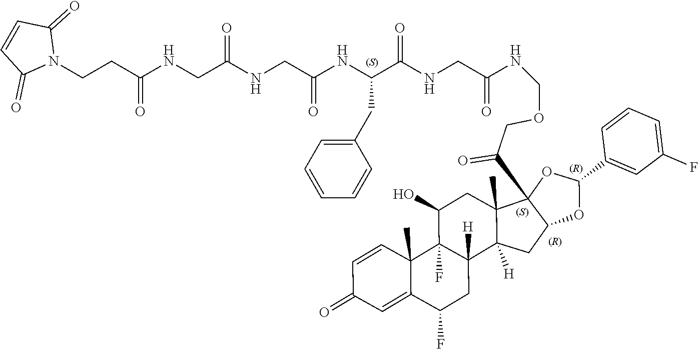

II-77

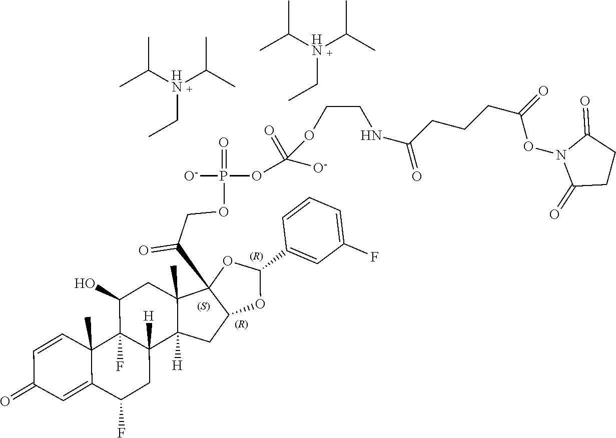

II-78

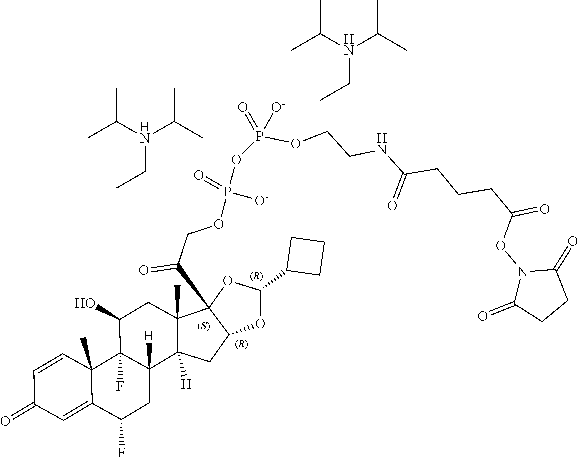

II-79

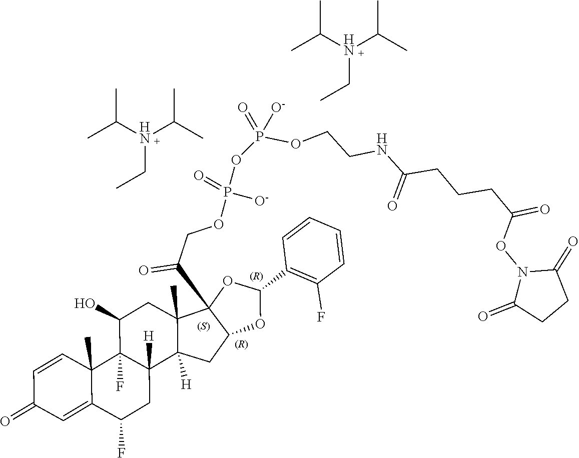

II-80

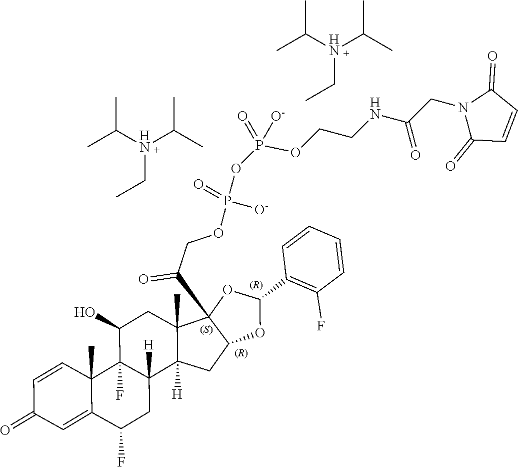

II-81

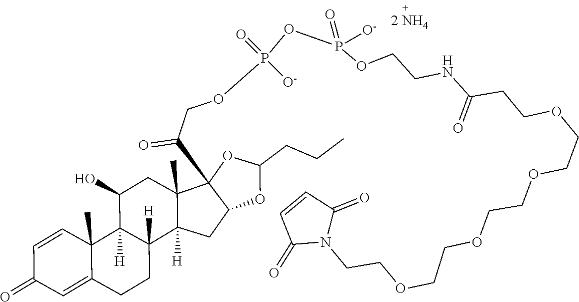

II-82

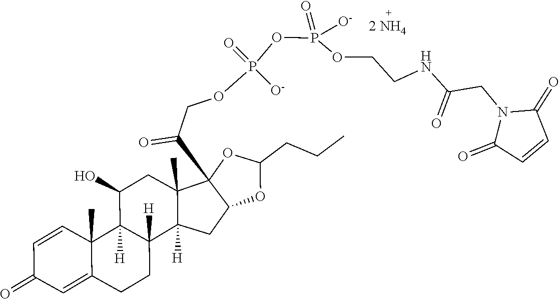

II-83

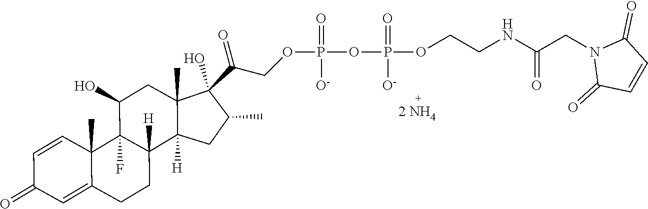

II-84

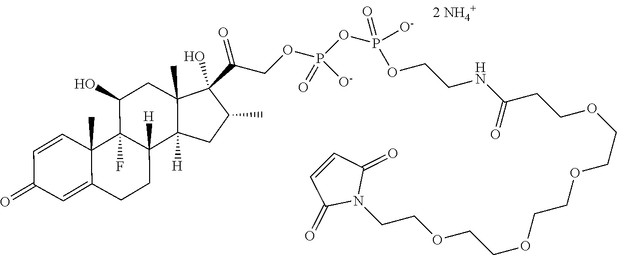

II-85

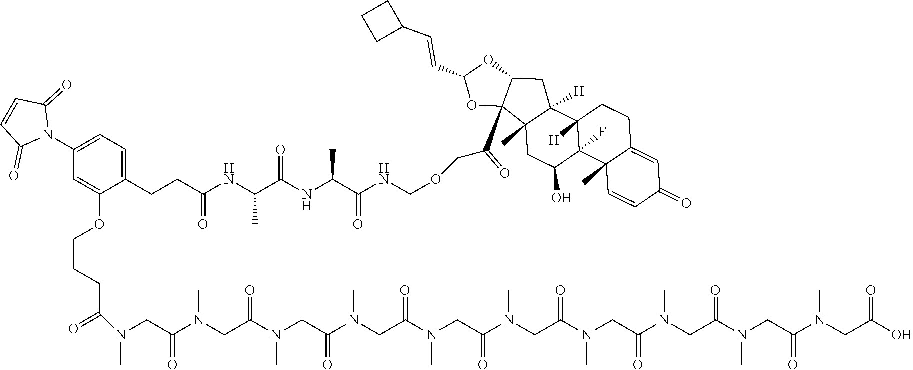

II-86

In some embodiments, the conjugate of the disclosure comprises a compound that has the structure of Formula II-1:

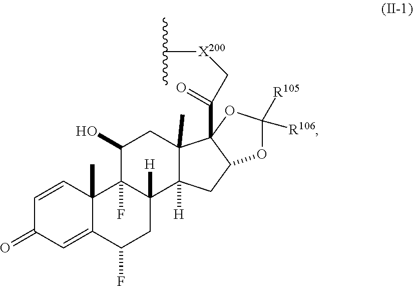

or a pharmaceutically acceptable salt thereof,
wherein
R¹⁰⁵ is C₄₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl is substituted with 1, 2, or 3 R¹¹⁰, and the heterocyclyl is substituted with 0, 1, 2 or 3 R¹¹⁰;
each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹²,
   —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹²,
   —(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹¹ is independently C₁₋₆ alkyl, C₁₋₆ alkoxy, C₁₋₆ haloalkyl, C₁₋₆ haloalkoxy, or halogen;
each R¹¹² is independently H, C₁₋₆ alkyl, C₁₋₆ haloalkyl, or phenyl;
each R¹¹³ is independently —S(O)₂(C₁₋₆ alkyl) or —N(R¹¹⁵)₂;
each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶ or —S(O)₂N(R¹¹⁶)₂;
each R¹¹⁵ and R¹¹⁶ is independently H, C₁₋₆ alkyl, or C₁₋₆ haloalkyl;
X²⁰⁰ is —O— or —NH— and is covalently attached to the linker;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, the conjugate of the disclosure comprises a compound that has the structure of Formula II-1a or II-Ib:

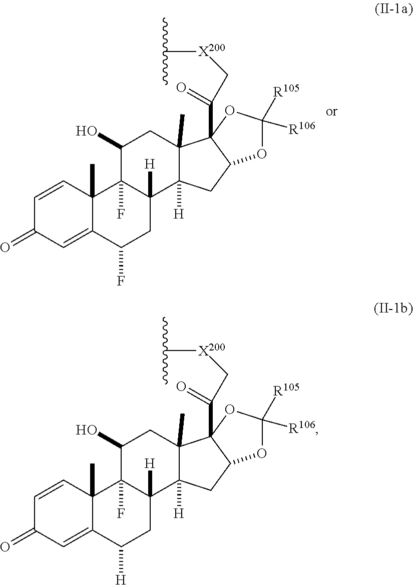

or a pharmaceutically acceptable salt thereof,
wherein
R¹⁰⁵ is C₄₋₆ alkyl, C₂₋₆ alkenyl, C₂₋₆ alkynyl, C₁₋₆ haloalkyl, phenyl, —(C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl is substituted with 1, 2, or 3 R¹¹⁰, and the heterocyclyl is substituted with 0, 1, 2 or 3 R¹¹⁰;
each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹², —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹², —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹¹ is independently C₁₋₆ alkyl, C₁₋₆ alkoxy, C₁₋₆ haloalkyl, C₁₋₆ haloalkoxy, or halogen;
each R¹¹² is independently H, C₁₋₆ alkyl, C₁₋₆ haloalkyl, or phenyl;
each R¹¹³ is independently —S(O)₂(C₁₋₆ alkyl) or —N(R¹¹⁵)₂;
each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶, —S(O)₂N(R¹¹⁶)₂ or R³⁰⁰;
each R¹¹⁵ and R¹¹⁶ is, at each occurrence, independently H, C₁₋₆ alkyl, C₁₋₆ haloalkyl or R³⁰⁰;
X²⁰⁰ is —O— or —NH— and is covalently attached to the linker; and
R³⁰⁰ has one of the following structures:

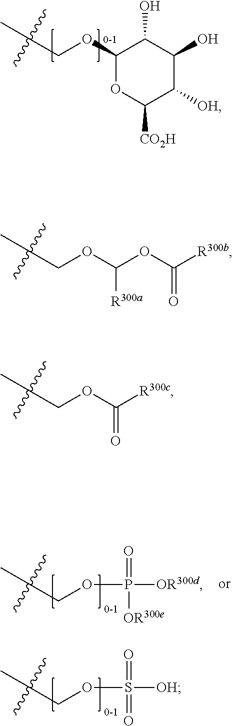

wherein
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl;
   R^{300e} is H or C₁₋₆ alkyl; and
wherein the heteroaryl in each instance is a 5- to 10-membered heteroaryl having 1, 2, or 3 heteroatoms selected from N, O, and S; and
   the heterocyclyl in each instance is a 4- to 10-membered heterocyclyl having 1, 2, or 3 heteroatoms selected from N, O, and S.

In some embodiments, X²⁰⁰ is —O— and is covalently attached to the linker.

In some embodiments, the conjugate of the disclosure comprises a compound that has the structure of Formula II-2:

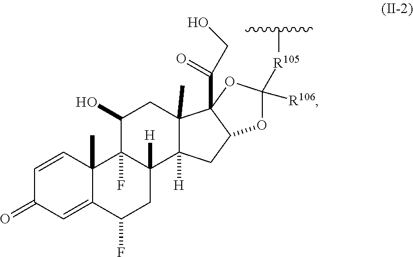

or a pharmaceutically acceptable salt thereof,
wherein
R¹⁰⁵ is C₄₋₆ alkyl, C₂₋₆ alkenyl, C₂₋₆ alkynyl, C₁₋₆ haloalkyl, phenyl, (C₁₋₆ alkylene)-phenyl, heteroaryl, —(C₁₋₆ alkylene)-heteroaryl, C₃₋₈ cycloalkyl, or heterocyclyl, wherein the alkyl or alkenyl is substituted with 0, 1, 2, or 3 R¹⁰⁷, the alkynyl is substituted with 0, 1, 2, or 3 R¹⁰⁸, the phenyl is substituted with 0, 1, 2 or 3 R¹⁰⁹, and the -alkylene-phenyl, heteroaryl, -alkylene-heteroaryl, cycloalkyl or heterocyclyl is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkyl or a heterocyclyl, wherein the cycloalkyl is substituted with 1, 2, or 3 R¹¹⁰, and the heterocyclyl is substituted with 0, 1, 2 or 3 R¹¹⁰; and
R¹⁰⁵ is covalently attached to the linker;
each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹²,
   N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹²,
   N(R¹¹²)₂, —(CO)R¹¹², or —S(O)₂R¹¹², wherein the alkynyl or haloalkoxy is substituted with 0, 1, 2, or 3 R¹¹³, and the phenyl, -alkylene-phenyl, heteroaryl, -alkylene-heteroaryl, or heterocyclyl is substituted with 0, 1, 2, or 3 R¹¹⁴;
each R¹¹¹ is independently C₁₋₆ alkyl, C₁₋₆ alkoxy, C₁₋₆ haloalkyl, C₁₋₆ haloalkoxy, or halogen;
each R¹¹² is independently H, C₁₋₆ alkyl, C₁₋₆ haloalkyl, or phenyl;
each R¹¹³ is independently —S(O)₂(C₁₋₆ alkyl) or —N(R¹¹⁵)₂;
each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶, or —S(O)₂N(R¹¹⁶)₂;
each R¹¹⁵ and R¹¹⁶ is independently H, C₁₋₆ alkyl, or C₁₋₆ haloalkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, the conjugate of the disclosure comprises a compound that has the structure of Formula II-3a or II-3b:

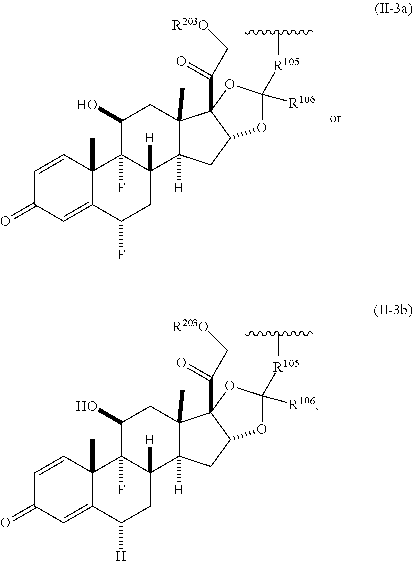

or a pharmaceutically acceptable salt thereof,
wherein
R¹⁰⁵ is C₄₋₆ alkylene, C₂₋₆ alkenylene, C₂₋₆ alkynylene, C₁₋₆ haloalkylene, phenylene, —(C₁₋₆ alkylene)-phenylene, heteroarylene, —(C₁₋₆ alkylene)-heteroarylene, C₃₋₈ cycloalkylene, or heterocyclylene, wherein the alkylene or alkenylene is substituted with 0, 1, 2, or 3 R¹⁰⁷, the alkynylene is substituted with 0, 1, 2, or 3 R¹⁰⁸, the phenylene is substituted with 0, 1, 2 or 3 R¹⁰⁹, and the -alkylene-phenylene, heteroarylene, -alkylene-heteroarylene, cycloalkylene or heterocyclylene is substituted with 0, 1, 2 or 3 R¹¹⁰;
R¹⁰⁶ is H;
or, alternatively, R¹⁰⁵ and R¹⁰⁶ taken together combine to form a C₃₋₈ cycloalkylene or a heterocyclylene, wherein the cycloalkylene is substituted with 1, 2, or 3 R¹¹⁰, and the heterocyclylene is substituted with 0, 1, 2 or 3 R¹¹⁰; and
R¹⁰⁵ is covalently attached to the linker;
each R¹⁰⁷ and R¹⁰⁸ is independently C₁₋₆ alkyl, C₂₋₆ alkoxyalkyl, C₁₋₆ haloalkoxy, phenyl, heteroaryl, C₃₋₈ cycloalkyl, heterocyclyl, or halogen, wherein the phenyl, heteroaryl, cycloalkyl, or heterocyclyl is substituted with 0, 1, 2, or 3 R¹¹¹;
each R¹⁰⁹ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₂₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹², —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹⁰ is independently C₁₋₆ alkyl, C₂₋₆ alkenyl, C₂₋₆ alkynyl, C₂₋₆ alkoxyalkyl, —(C₁₋₆ alkylene)-OR¹¹², —(C₁₋₆ alkylene)-N(R¹¹²)₂, C₁₋₆ haloalkyl, C₁₋₆ haloalkoxy, phenyl, —(C₁₋₆ alkylene)-phenyl, heteroaryl, —(C₁₋₆ alkylene)-heteroaryl, heterocyclyl, —(C₁₋₆ alkylene)-heterocyclyl, halogen, —N₃, —OR¹¹², —N(R¹¹²)₂, —(CO)R¹¹², or —S(O)₂R¹¹², wherein the alkynyl or haloalkoxy is substituted with 0, 1, 2, or 3 R¹¹³ and the phenyl, -alkylene-phenyl, heteroaryl, -alkylene-heteroaryl, or heterocyclyl is substituted with 0, 1, 2, or 3 R¹¹⁴;
each R¹¹¹ is independently C₁₋₆ alkyl, C₁₋₆ alkoxy, C₁₋₆ haloalkyl, C₁₋₆ haloalkoxy, or halogen;
each R¹¹² is independently H, C₁₋₆ alkyl, C₁₋₆ haloalkyl, or phenyl;
each R¹¹³ is independently —S(O)₂(C₁₋₆ alkyl) or —N(R¹¹⁵)₂;
each R¹¹⁴ is independently C₁₋₆ alkyl, —(C₁₋₆ alkyl)-OR¹¹⁶, —(C₁₋₆ alkyl)-N(R¹¹⁶)₂, —OR¹¹⁶, —N(R¹¹⁶)₂, —N(R¹¹⁶)(CO)R¹¹⁶, —N(R¹¹⁶)(CO)OR¹¹⁶, —N(R¹¹⁶)S(O)₂R¹¹⁶, —C(O)R¹¹⁶, —S(O)₂R¹¹⁶, —S(O)₂N(R¹¹⁶)₂, or R³⁰⁰;
each R¹¹⁵ and R¹¹⁶ is, at each occurrence, independently H, C₁₋₆ alkyl, C₁₋₆ haloalkyl or R³⁰⁰;
R²⁰³ is H or R³⁰⁰; and
R³⁰⁰ has one of the following structures:

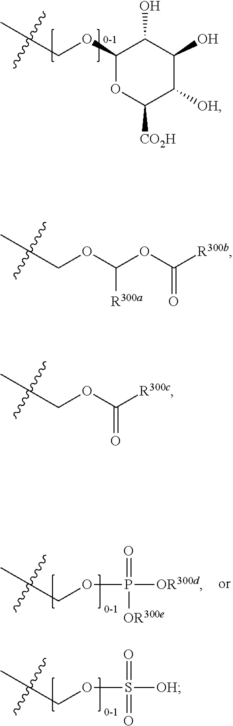

wherein
   R^{300a} is H or C₁₋₆ alkyl;
   R^{300b} is C₁₋₆ alkyl or C₁₋₆ alkoxy;
   R^{300c} is H, C₁₋₆ alkyl, —CH₂OH, or C₁₋₆ alkoxy;
   R^{300d} is H or C₁₋₆ alkyl;
   R^{300e} is H or C₁₋₆ alkyl; and
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

In some embodiments, R¹⁰⁵ comprises —NH— that is covalently attached to the linker.

As understood in the art, the conjugate of the present disclosure prepared from covalent attachment of a thiol group on a binding protein to a maleimide-containing linker-payload compound of the present invention can have a structure:

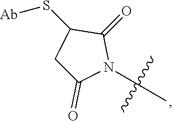

or the conjugate can have a ring-opened structure:

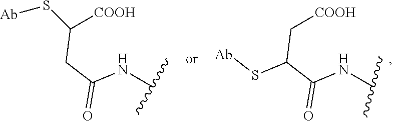

wherein Ab is a binding protein, and [symbol] represents the attachment to the remainder of the linker-payload. Representations of the conjugate of the present disclosure in one format, e.g., showing a sulfide attached to a succinimide ring, is not limiting and is intended to capture all possible structures, including the ring-opened structures described above. Accordingly, in one illustrative example with linker-payload described herein, the conjugate can have the structure:

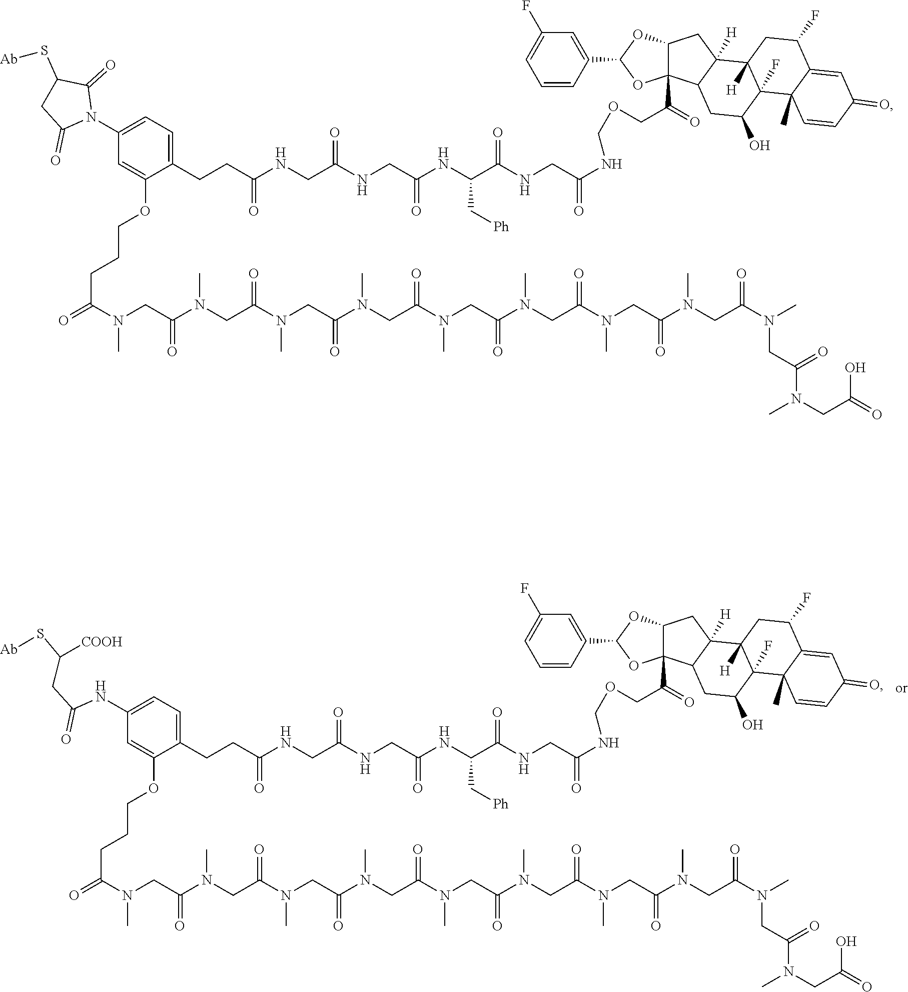

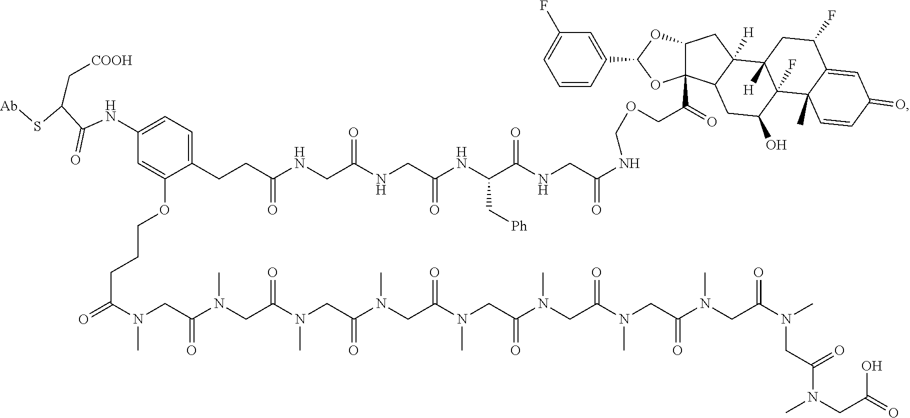

wherein Ab is a binding protein.

In some embodiments, the conjugate of the present disclosure has a structure according to a conjugate described in the Examples.

The conjugates described herein can be synthesized by coupling the linker-payloads described herein with a binding agent, e.g., antibody under standard conjugation conditions (see, e.g., US patent publications 2018/0155389, 2019/0167804, 2019/0209702, 2019/0262465, 2019/0367631, and 2021/0040144; and articles Drug Deliv. 2016 June; 23(5):1662-6; AAPS Journal, Vol. 17, No. 2, March 2015; and Int. J Mol. Sci. 2016, 17, 561; the entireties of which are incorporated herein by reference). Linker-payloads are synthetic intermediates comprising the compound of Structure (I) or (II) of interest and linking moiety that ultimately serves as the moiety (or portion thereof) that connects the binding agent with the compound of Structure (I) or (II). Linker-payloads comprise a reactive group that reacts with the binding agent to form the conjugates described herein. When the binding agent is an antibody, the antibody can be coupled to a linker-payload via one or more cysteine, lysine, or other residue of the antibody. Linker-payloads can be coupled to cysteine residues, for example, by subjecting the antibody to a reducing agent, e.g., dithiotheritol, to cleave the disulfide bonds of the antibody, purifying the reduced antibody, e.g., by gel filtration, and subsequently reacting the antibody with a linker-payload containing a reactive moiety, e.g., a maleimido group. Suitable solvents include, but are not limited to water, DMA, DMF, and DMSO. Linker-payloads containing a reactive group, e.g., activated ester or acid halide group, can be coupled to lysine residues. Suitable solvents include, but are not limited to water, DMA, DMF, and DMSO. Conjugates can be purified using known protein techniques, including, for example, size exclusion chromatography, dialysis, and ultrafiltration/diafiltration.

In some embodiments, a conjugate of the present invention can be attached to a linker-payload compound via cysteine-based bio-conjugation. A binding protein can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol (DTT) or tris(2-carboxyethyl)phosphine (TCEP). The resultant solution can be stirred for an appropriate amount of time and temperature to effect the desired reduction. A linker-payload compound, such as a GR agonist-linker described herein, e.g., a linker covalently attached to a compound of Formula I, can be added as a solution with stirring. Dependent on the physical properties of the linker-payload compound, a co-solvent can be introduced prior to the addition of the linker-payload compound to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload compound can be removed by applicable methods and the conjugate of the present invention can be exchanged into the desired formulation buffer. Such conjugates can be synthesized starting with a binding protein such as an antibody or mAb, and linker-payload compound, e.g., 7 equivalents, using the general conditions described in the Conjugation Reaction Scheme below. Monomer content and drug-antibody ratios can be determined by methods described herein and known in the art.

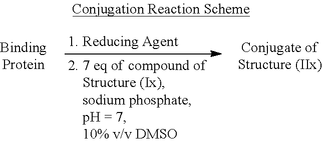

### V. Compositions

Binding protein conjugates and compositions thereof of this disclosure are useful as, or may be used in, pharmaceutical compositions for administration to a subject in need thereof. In some embodiments, pharmaceutical compositions comprise a conjugate of this disclosure and one or more pharmaceutically acceptable carriers, diluents, or excipients. In some embodiments, a pharmaceutical composition comprises at least one of the conjugates of this disclosure and further comprises one or more of a buffer, antibiotic, steroid, carbohydrate, second drug (e.g., anti-inflammatory drug), radiation, polypeptide, chelator, adjuvant, or preservative.

Pharmaceutical compositions may be formulated using one or more physiologically acceptable carriers comprising excipients and auxiliaries. Formulations may be modified depending upon the route of administration chosen. Pharmaceutical compositions comprising a conjugate may be manufactured, for example, by lyophilizing mixing, dissolving, emulsifying, encapsulating, or entrapping a conjugate of this disclosure. Pharmaceutical compositions may also include conjugates of this disclosure in a free-base form or a pharmaceutically acceptable salt form.

Methods for formulation of the conjugates may include formulating any of the conjugates with one or more inert, pharmaceutically acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the conjugates may be lyophilized or in powder form for re-constitution with a suitable vehicle, e.g., sterile pyrogen-free water, before use.

Pharmaceutical compositions of the conjugates provided herein may comprise at least one active ingredient (e.g., a conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nanoparticles, and nanocapsules) or in macroemulsions.

Pharmaceutical compositions may comprise more than one active compound (e.g., a compound, salt or conjugate and other agents) as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, or cardioprotectant. Such molecules may be present in combination in amounts that are effective for the purpose intended.

The compositions and formulations may be sterilized. Sterilization may be accomplished by filtration through sterile filtration.

Compositions of this disclosure may be formulated for administration as an injection. Non-limiting examples of formulations for injection may include a sterile suspension, solution, or emulsion in oily or aqueous vehicles. Suitable oily vehicles may include lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, a composition of this disclosure may be lyophilized or in powder form for reconstitution with a suitable vehicle, e.g., sterile pyrogen-free water, before use.

For parenteral administration, the conjugates may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations may be prepared for storage by mixing a conjugate of this disclosure with a pharmaceutically acceptable carrier, diluents, excipient, or a stabilizer. This formulation may be a lyophilized formulation or an aqueous solution. Acceptable carriers, diluents, excipients, or stabilizers may be nontoxic to recipients at the dosages and concentrations used. Acceptable carriers, diluents, excipients, or stabilizers may include buffers, such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; or non-ionic surfactants or polyethylene glycol.

In some embodiments, an aqueous formulation of a conjugate provided herein, such as for subcutaneous administration, has a pH from 4-5.2. The aqueous formulation may comprise one or more excipients, such as one or more buffering agents, one or more lyoprotectants, and the like. In some embodiments, the pH of the formulation is from 4-5.1, 4.1-5.1, 4.2-5.1, 4.3-5.1, 4.4-5.1, 4.5-5.1, 4-5, 4.1-5, 4.2-5, 4.3-5, 4.4-5, or 4.5-5. In some embodiments, the formulation comprises at least one buffer. In some embodiments, the buffer may be selected from histidine, citrate, aspartate, acetate, phosphate, lactate, tromethamine, gluconate, glutamate, tartrate, succinate, malic acid, fumarate, α-ketoglutarate, and combinations thereof. In some embodiments, the buffer is at least one buffer selected from histidine, citrate, aspartate, acetate, and combinations thereof. In some embodiments, the buffer is a combination of histidine and aspartate. In some embodiments, the total concentration of the buffer in the aqueous formulation is at least 0.01 mM, 0.1 mM, 1 mM, 5 mM, or 10 mM. In some embodiments, the total concentration of the buffer in the aqueous formulation is between 10 mM and 40 mM. In some embodiments, the total concentration of the buffer in the aqueous formulation is between 15 mM and 30 mM. In some embodiments, the total concentration of the buffer in the aqueous formulation is between 15 mM and 25 mM. In some embodiments, the total concentration of the buffer in the aqueous formulation is 20 mM or about 20 mM.

In some embodiments, the aqueous formulation comprises at least one lyoprotectant. In some embodiments, the at least one lyoprotectant is selected from sucrose, arginine, glycine, sorbitol, glycerol, trehalose, dextrose, alpha-cyclodextrin, hydroxypropyl beta-cyclodextrin, hydroxypropyl γ-cyclodextrin, proline, methionine, albumin, mannitol, maltose, dextran, and combinations thereof. In some embodiments, the lyoprotectant is sucrose. In some embodiments, the total concentration of lyoprotectant in the aqueous formulation is 3-12%, such as 5-12%, 6-10%, 5-9%, 7-9%, or 8%.

In some embodiments, the aqueous formulation comprises at least one surfactant. Exemplary surfactants include polysorbate 80, polysorbate 20, poloxamer 88, and combinations thereof. In some embodiments, the aqueous formulation comprises polysorbate 80. In some embodiments, the total concentration of the at least one surfactant is 0.01%-0.1%, such as 0.01%-0.05%, 0.01%-0.08%, or 0.01%-0.06%, 0.01%-0.04%, 0.01%-0.03%, or 0.02%.

In some embodiments, the concentration of the conjugate in the aqueous formulation is 1 mg/mL-200 mg/mL, such as 10 mg/mL-160 mg/mL, 10 mg/mL-140 mg/mL, 10 mg/mL-120 mg/mL, 20 mg/mL-120 mg/mL, or 30 mg/mL-120 mg/mL, or 40 mg/mL-120 mg/mL, or 40 mg/mL-100 mg/mL. In some embodiments, the concentration of the conjugate in the aqueous formulation is 10 mg/mL-140 mg/mL or 40 mg/mL-140 mg/mL.

Pharmaceutical formulations may have conjugates of this disclosure with an average ratio of the compound of the present disclosure, e.g., a compound of Formula I or Formula II, to binding protein (referred to herein as a drug-to-antibody ratio, or DAR) that ranges from 1 to about 20, from 1 to about 10, from 1 to about 8, from 1 to about 6, from 1 to about 5, from 1 to about 3, from 2 to about 8, from 2 to about 6, from 2 to about 5, from 2 to about 4, from about 3 to about 8, from about 3 to about 6, or from about 3 to about 5, wherein the drug is a compound of the present disclosure. In some embodiments, the average ratio of the compound of the present disclosure to binding protein of conjugates in a pharmaceutical formulation may range from 1 to 20, from 1 to 10, from 1 to 8, from 1 to 6, from 1 to 5, from 1 to 3, from 2 to 8, from 2 to 6, from 2 to 5, from 2 to 4, from 3 to 8, from 3 to 6, or from 3 to 5. In some embodiments, the average ratio of the compound of the present disclosure to binding protein in the conjugate is 2 or about 2, 3 or about 3, 4 or about 4, 5 or about 5, 6 or about 6, 7 or about 7, or 8 or about 8.

### VI. Methods of Treatment and/or Uses

The present disclosure provides methods of treating or preventing diseases or conditions (e.g., autoimmune, or inflammatory conditions, also referred to inflammation herein) in a subject in need thereof, comprising administering to the subject an effective amount of binding protein conjugates or compositions thereof disclosed herein. Similarly, the present disclosure provides use of binding protein conjugates or compositions thereof disclosed herein in the manufacture of a medicament for treating or preventing diseases or conditions, such as autoimmune or inflammatory conditions.

As used herein, the term “effective amount” or “effective dose” refers to a quantity of a binding protein conjugate or composition thereof sufficient to achieve a desired (e.g., beneficial) effect in a subject being treated with that compound, conjugate, or composition thereof, such as an amount sufficient to result in amelioration of one or more symptoms of the disease being treated in a statistically significant manner, delaying worsening of a progressive disease in a statistically significant manner, or preventing onset of additional associated symptoms or diseases in a statistically significant manner, or any combination thereof. In certain embodiments, an effective amount of a binding protein conjugate or composition thereof is an amount sufficient to inhibit or treat the disease with minimal to no toxicity in the subject, excluding the presence of one or more adverse side effects. An effective amount or dose can be administered one or more times over a given period of time. An effective amount or dose can depend on the purpose of the treatment and can be ascertainable by one skilled in the art based on a subject's needs. When referring to an individual active ingredient, administered alone, an effective amount or dose refers to that ingredient alone. When referring to a combination, an effective amount or dose refers to combined amounts of the active ingredients that result in the therapeutic effect, whether administered serially or simultaneously.

Conjugates, such as binding protein conjugates, compositions thereof, and methods of this disclosure are useful for treatment or prevention of disease in one or more subject species including humans, mammals, non-human mammals, non-human primates, dogs, cats, rodents, mice, hamsters, cows, birds, chickens, fish, pigs, horses, goats, sheep, rabbits, guinea pigs and any combination thereof. In preferred embodiments, binding protein conjugates, compositions thereof, and methods of this disclosure are useful for treatment or prevention of a disease in a human.

In some embodiments of the methods of the disclosure, a subject in need of treatment has been diagnosed with the disease or condition. In some embodiments, the subject has been treated with another therapy. In some embodiments, the subject is resistant to another therapy or has relapsed following administration of another therapy, and therefore, is in need of treatment by providing or administrating to the subject one or more of a binding protein conjugate or a composition thereof of this disclosure.

In some embodiments of the methods of the disclosure, the subject is at risk of developing a disease or condition as a result of genetic or environmental risk factors. In some embodiments, a subject carries one or more genetic markers for a disease or condition of the disclosure, including autoimmune conditions (related to inflammation). Alternatively, or in addition, in some embodiments, a subject is exposed to an infectious agent (e.g., a virus, bacterium, parasite or microbe), an allergen or irritant, or a carcinogen (e.g., radiation, mutagen, viral infection).

In some embodiments of the methods of this disclosure, a therapeutically effective amount of one or more of a binding protein conjugate or composition thereof of this disclosure is administered to a subject in need thereof. In practicing the methods described herein, therapeutically effective amounts of the binding protein conjugates and pharmaceutical compositions thereof can be administered to a subject in need thereof, often for treating or preventing a condition or progression thereof. The binding protein conjugates and pharmaceutical compositions thereof of this disclosure can affect the physiology of the subject, such as the immune system, an inflammatory response, or other physiologic affect. A therapeutically effective amount can vary depending on the severity of the disease, the age and relative health of the subject, the potency of the compounds used, and other factors.

Conjugates and compositions of the disclosure may be administered to a subject by any route, including intravenous, intraarterial, subcutaneous, subdural, intramuscular, intracranial, intrasternal, intratumoral, intraspinal, intrathecal, or intraperitoneal injection or infusion.

Conjugates and compositions of the disclosure may be administered to a subject in one or more doses, one or more injections/infusions, one or more cycles, or one or more administrations. Conjugates and compositions of the disclosure may be administered to a subject daily, weekly, or monthly. Conjugates and compositions of the disclosure may be administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. Conjugates and compositions of the disclosure may be administered to a subject once every 1, 2, 3, or 4 weeks. Conjugates and compositions of the disclosure may be administered to a subject once every 1, 2, 3, 4, 5, 6, 7, 8, 9, 10, 11, or 12 months.

Conjugates and compositions of this disclosure may be administered to a subject in one or more doses, wherein each dose or the total dose per treatment cycle may comprise between 0.1 mg/kg and 100 mg/kg, inclusive of the endpoints. Conjugates and compositions of this disclosure may be administered to a subject in one or more doses, wherein each dose or the total dose per treatment cycle may comprise between about 0.1 mg/kg and about 100 mg/kg, inclusive of the endpoints. In some embodiments, each dose or the total dose per treatment cycle may comprise between 1 mg/kg and 15 mg/kg, inclusive of the endpoints. In some embodiments, each dose or the total dose per treatment cycle may comprise between about 1 mg/kg and about 15 mg/kg, inclusive of the endpoints. In some embodiments, each dose or the total dose per treatment cycle may comprise between 1 mg/kg and 10 mg/kg, inclusive of the endpoints. In some embodiments, each dose or the total dose per treatment cycle may comprise between about 1 mg/kg and about 10 mg/kg, inclusive of the endpoints. In some embodiments, each dose or the total dose per treatment cycle may comprise at least 1, 2, 3, 4, 5, 6, 7, 8, 9, 10 mg/kg. In some embodiments, each dose or the total dose per treatment cycle may comprise 0.1, 0.5, 1, 2, 3, 4, 5, 6, 7, 8, 9, 10, 11, 12, 13, 14, 15 mg/kg or any number of mg/kg in between. In some embodiments, each dose or the total dose per treatment cycle may comprise about 0.1, about 0.5, about 1, about 2, about 3, about 4, about 5, about 6, about 7, about 8, about 9, about 10, about 11, about 12, about 13, about 14, about 15 mg/kg or any number of mg/kg in between. In some embodiments, the treatment cycle comprises one or more treatment administrations and one or more periods of observation. In some embodiments, the treatment cycle comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. In some embodiments, the treatment cycle comprises at least 1, 2, 3, or 4 weeks. In some embodiments, the treatment cycle comprises at least 1, 2, 3, 4, 5, 6, 7, 8, 9, 10, 11, or 12 months.

In various embodiments, methods of treating a disease mediated by GR activitation are provided, comprising administering to a subject in need thereof an effective amount of a conjugate or composition thereof provided herein. In some embodiments, the disease or condition is an autoimmune condition or inflammation. The conjugates, compositions thereof, and methods of this disclosure are useful for specifically targeting the signaling or activities of GR, a component of one of the related pathways, or combinations thereof, such as increasing signaling by GR, a component of one of the related pathways, or combinations thereof.

The conjugates, compositions thereof, and methods of this disclosure may be used in combination with a second therapeutic agent for treating or preventing diseases, such as autoimmune conditions or inflammation. In some embodiments of the disclosure, including those in which the conjugates and compositions thereof of this disclosure are intended for the treatment or prevention of inflammation or an autoimmune condition, a second therapeutic agent comprises one or more of a second compound, conjugate, or pharmaceutical composition of this disclosure; an anti-inflammatory composition; a steroid composition, a nonsteroidal anti-inflammatory drug (NSAID) composition, a cyclooxygenase (COX) enzyme (e.g., COX1 or COX2 inhibitor) composition; and a regulatory T-cell antagonist composition. In some embodiments, a second therapeutic agent comprises an anti-inflammatory agent. In some embodiments, an anti-inflammatory agents comprises a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In certain embodiments, anti-inflammatory agents such as ruxolitinib or anakinra are used. Other anti-inflammatory agents for use in a combination therapy of the present disclosure include non-steroidal anti-inflammatory drugs (NSAIDS). Examples of anti-inflammatory drugs include anakinra, aspirin, diclofenac, diflunisal, etodolac, ibuprofen, indomethacin, ketoprofen, ketorolac, nabumetone, naproxen, oxaprozin, piroxicam, ruxolitinib, salsalate, sulindac, tipredane, tolmetin, and triamcinolone acetonide.

In some embodiments, a second therapeutic agent comprises an immunosuppressant (e.g., 6-mercaptopurine, adalimumab, azathioprine, basiliximab, certolizumab pegol, cyclosporin, daclizumab, infliximab, mercaptopurine, methotrexate, methotrexate, mycophenolate mofetil, natalizumab, sirolimus, tacrolimus, ustekinumab, and vedolizumab).

In some embodiments, a second therapeutic agent comprises a pain reliever. In some embodiments, a second therapeutic agent comprises an analgesic (e.g., codeine, dihydromorphine, ergotamine, fentanyl, or morphine).

A second therapeutic agent may be administered simultaneously with a conjugate of this disclosure or a pharmaceutical composition thereof. Alternatively, a second therapeutic agent and a conjugate of the present disclosure or a pharmaceutical composition thereof may be administered sequentially.

A second therapeutic agent may be administered in the same route as a conjugate provided herein or a pharmaceutical composition comprising the conjugate, such as both intravenously or both subcutaneously. Alternatively, a second therapeutic agent and a conjugate provided herein, or a pharmaceutical composition may be administered via different routes, such as one intravenously and the other subcutaneously.

Examples of autoimmune conditions include acute disseminated encephalomyelitis; anti-N-Methyl-D-Aspartate (Anti-NMDA) receptor encephalitis; Addison's disease; adult-onset Still's disease; anti-glomerular basement membrane nephritis; antiphospholipid syndrome; aplastic anemia; autoimmune enteropathy; autoimmune hepatitis; autoimmune hemolytic anemia; autoimmune lymphoproliferative syndrome; autoimmune neutropenia; autoimmune oophoritis; autoimmune polyendocrine syndrome (APS) type 1; autoimmune polyendocrine syndrome (APS) type 2; autoimmune polyendocrine syndrome (APS) type 3; autoimmune pancreatitis; autoimmune neutropenia; autoimmune thrombocytopenic purpura; autoimmune thyroiditis; autoimmune vasculitis; balo concentric sclerosis; bullous pemphigoid; celiac disease; chronic inflammatory demyelinating polyneuropathy; cold agglutinin disease; dermatomyositis; dermatitis herpetiformis; drug-induced lupus; epidermolysis bullosa acquisita; Evans syndrome; felty syndrome; granulomatosis with polyangiitis (GPA); Graves' disease; Guillain-Barre syndrome; Hashimoto's thyroiditis; fnflammatory bowel disease (IBD): Crohn's disease, Ulcerative colitis; Juvenile arthritis; Kawasaki disease; Linear IgA disease; Lambert-Eaton myasthenic syndrome; lupus vasculitis; mixed connective tissue disease (MCTD); multiple sclerosis (MS); myasthenia gravis (MG); Ord's thyroiditis; pemphigus vulgaris; pernicious anemia; primary biliary cholangitis (PBC); psoriasis; psoriatic arthritis (PsA); relapsing polychondritis; rheumatoid arthritis (RA); rheumatic fever; rheumatoid vasculitis; Sjogren's syndrome (SS); systemic lupus erythematosus (SLE); type 1 diabetes; undifferentiated connective tissue disease (UCTD); and vasculitis.

Examples of inflammatory conditions include actinic conjunctivitis, active hepatitis, acute bronchitis, acute hemorrhagic conjunctivitis, acute pancreatitis, adenoiditis, adrenalitis, alcoholic hepatitis, allergic reactions, allergies, appendicitis, arachnoiditis, arteritis, arthritis, arthritis and other joint diseases, ascending cholangitis, asthma, atherosclerosis, atrophic vaginitis, balanitis, balanitis circinata, balanoposthitis, behçet's disease, blepharitis, bronchiolitis, bronchitis, bursitis, caecitis, calcific tendinitis, capillaritis, capsulitis, cardiovascular disease (CVD), carditis, catarrh, cellulitis, cerebral vasculitis, cervicitis, cheilitis, chemical colitis, chemosis, cholecystitis, chondritis, chorioamnionitis, chorioretinitis, chronic actinic dermatitis, chronic atrophic rhinitis, chronic deciduitis, chronic obstructive pulmonary disease (COPD), chronic peptic ulcer, climber's finger, climbing injuries, cold compression therapy, colitis, common cold, condensing osteitis, conjunctivitis, constrictive pericarditis, crohn's disease, cryptitis, cuffitis, cystitis, cytomegalovirus esophagitis, dacryoadenitis, dactylitis, dermatitis, dermatomyositis, dermatopolymyositis, diabetes, duodenal lymphocytosis, duodenitis, encephalitis, endocarditis, endometritis, endotheliitis, enteritis, enterocolitis, enterotoxin type b, enthesitis, eosinophilic fasciitis, epicondylitis, epididymitis, epiploic appendagitis, episcleritis, esophagitis, exposure keratopathy, fasciitis, folliculitis, funisitis, gastritis, gastroenteritis, gingivitis, gingivostomatitis, glomerulonephritis, glossitis, gnathitis, golfer's vasculitis, granuloma, hepatitis, hereditary gingival fibromatosis, herpetic gingivostomatitis, hidradenitis, hidradenitis suppurativa, hypophysitis, ileitis, immune-mediated inflammatory diseases, inclusion body myositis, infections associated with diseases, inflammatory bowel disease (IBD), inguinal lymphadenopathy, insulitis, intertrigo, Kawasaki disease, keratic precipitate, keratitis, keratoconjunctivitis, knee arthritis, labyrinthitis, laryngitis, ligneous conjunctivitis, lymphadenitis, lymphadenopathy, lymphangitis, lymphocytic colitis, mastitis, mastoiditis, mediastinitis, membranous glomerulonephritis, meningitis, metallosis, multifocal stenosing ulceration of the small intestine, multisystem inflammatory syndrome, multisystem inflammatory syndrome in children, musculoskeletal injury, myelitis, myocarditis, myopericarditis, myositis, myositis ossificans, necrotizing fasciitis, nephritis, nephritis (e.g., glomerulonephritis or pyelonephritis), neuritis, neurogenic inflammation, non-gonococcal urethritis, nonpuerperal mastitis, omasitis, omphalitis, omphalitis of newborn, oophoritis, ophthalmia, orchitis, ormdl sphingolipid biosynthesis regulator 3, Osgood-Schlatter disease, osteitis, osteitis pubis, osteochondritis, osteochondritis: osteitis/osteomyelitis, otitis externa, otitis media, pancreatitis, panniculitis, panophthalmitis, parametritis, paraproctitis, parathyroiditis, parotitis, pericarditis, perichondritis, perifolliculitis, perimyositis, perinephritis, periodic fever syndrome, periodontal disease, periodontitis, periostitis, peritonitis, pes anserine bursitis, pharyngitis, phlebitis, phlegmasia cerulea dolens, phlegmon, photophthalmia, plantar fasciitis, pleurisy, pleuritis, pneumonitis, polymyositis, posthitis, postvaccinal encephalitis, primary sclerosing cholangitis, proctitis, prostatitis, psoriasis, pulpitis, pyelonephritis, pylephlebitis, pyomyositis, reactive aldehyde species, reactive gastropathy, retinal vasculitis, retinitis, rheumatic fever, rheumatoid arthritis, rhinitis, salpingitis, scleritis, secondary sclerosing cholangitis, seminal vesiculitis, serpiginous choroiditis, sesamoiditis, sialadenitis, sialadenitis/parotitis, simple clinical colitis activity index, sinusitis, skip lesion, spondylitis, staphylococcal enteritis, stomatitis, subacromial bursitis, subacute sclerosing panencephalitis, superior limbic keratoconjunctivitis, synovitis, synovitis/tenosynovitis, systemic lupus, temporal arteritis, tendinitis, tendinopathy, tennis elbow, tenosynovitis, tertiary peritonitis, tetter, Theiler's disease, thrombophlebitis, thyroiditis, tonsillitis, tracheitis, transient synovitis, trigger finger, trigonitis, trochleitis, tuberculosis, type 3c (pancreatogenic) diabetes, ulcerative colitis and crohn's disease, ureteritis, urethritis, uveitis, vaginitis, vasculitis, and vulvitis.

In some embodiments, an autoimmune or inflammatory condition is selected from systemic lupus erythematosus (SLE), antiphospholipid (APL) syndrome, rheumatoid arthritis (RA), multiple sclerosis (MS), IgA-nephropathy, organ transplant (such as heart, liver, lung, bone marrow, kidney, or pancreas), psoriatic arthritis (PsA), ankylosing spondylitis (AS), Sjogren's syndrome (SS), and myasthenia gravis (MG).

In some embodiments, the autoimmune condition is selected from the group consisting of Addison's disease, aplastic anemia, autoimmune hepatitis, autoimmune vasculitis, celiac disease, chronic inflammatory demyelinating polyneuropathy, Graves' disease, Guillain-Barre syndrome, Hashimoto's thyroiditis, inflammatory bowel disease, multiple sclerosis (MS), myasthenia gravis (MG), pernicious anemia, primary biliary cirrhosis, psoriatic arthritis (PsA), rheumatoid arthristis (RA), Sjogren's syndrome (SS), systemic lupus erythematosus (SLE), type 1 diabetes, and vasculitis.

In some embodiments, the inflammatory condition is a chronic inflammatory condition. Examples of chronic inflammatory conditions include diabetes, cardiovascular disease (CVD), arthritis and other joint diseases, allergies, chronic obstructive pulmonary disease (COPD), psoriasis, and rheumatoid arthritis.

In some embodiments, the inflammatory condition is a condition of the nervous system. In some embodiments, the inflammatory condition is a condition of the central nervous system (e.g., encephalitis, myelitis, meningitis, and arachnoiditis), the peripheral nervous system (e.g., neuritis), the eye (e.g., dacryoadenitis, scleritis, episcleritis, keratitis, retinitis, chorioretinitis, blepharitis, conjunctivitis, and uveitis), or the ear (e.g., otitis externa, otitis media, labyrinthitis, and mastoiditis).

In some embodiments, the inflammatory condition is a condition of the cardiovascular system. In some embodiments, the inflammatory condition is carditis, endocarditis, myocarditis, pericarditis, vasculitis, arteritis, phlebitis, or capillaritis.

In some embodiments, the inflammatory condition is a condition of the respiratory system. In some embodiments, the inflammatory condition is a condition of the upper respiratory system (e.g., sinusitis, rhinitis, pharyngitis, and laryngitis) or of the lower respiratory system (e.g., tracheitis, bronchitis, bronchiolitis, pneumonitis, pleuritis, and mediastinitis).

In some embodiments, the inflammatory condition is a condition of the digestive system. In some embodiments, the inflammatory condition is a condition of the mouth (e.g., stomatitis, gingivitis, gingivostomatitis, glossitis, tonsillitis, sialadenitis/parotitis, cheilitis, pulpitis, and gnathitis), the gastrointestinal tract (e.g., esophagitis, gastritis, gastroenteritis, enteritis, colitis, enterocolitis, duodenitis, ileitis, caecitis, appendicitis, and proctitis), or the accessory digestive organs (e.g., hepatitis, ascending cholangitis, cholecystitis, pancreatitis, and peritonitis).

In some embodiments, the inflammatory condition is a condition of the integumentary system. In some embodiments, the inflammatory condition is carditis, dermatitis, folliculitis, cellulitis, or hidradenitis.

In some embodiments, the inflammatory condition is a condition of the musculoskeletal system. In some embodiments, the inflammatory condition is arthritis, dermatomyositis, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis, or chondritis.

In some embodiments, the inflammatory condition is a condition of the urinary system. In some embodiments, the inflammatory condition is nephritis (e.g., glomerulonephritis or pyelonephritis), ureteritis, cystitis, or urethritis.

In some embodiments, the inflammatory condition is a condition of the reproductive system. In some embodiments, the inflammatory condition is oophoritis, salpingitis, endometritis, parametritis, cervicitis, vaginitis, vulvitis, mastitis, orchitis, epididymitis, prostatitis, seminal vesiculitis, balanitis, posthitis, or balanoposthitis.

In some embodiments, the inflammatory condition is a condition related to pregnancy.

In some embodiments, the inflammatory condition is chorioamnionitis, funisitis, or omphalitis.

In some embodiments, the inflammatory condition is a condition of the endocrine system. In some embodiments, the inflammatory condition is insulitis, hypophysitis, thyroiditis, parathyroiditis, or adrenalitis.

In some embodiments, the inflammatory condition is a condition of the lymphatic system. In some embodiments, the inflammatory condition is lymphangitis or lymphadenitis.

In some embodiments, the inflammatory condition is selected from the group consisting of allergies, ankylosing spondylitis (AS), antiphospholipid (APL) syndrome, arthritis and other joint diseases, asthma, cardiovascular disease (CVD), chronic obstructive pulmonary disease (COPD), chronic peptic ulcer, dermatitis, diabetes, endometriosis, fatty liver disease, gout, hepatitis, inflammatory bowel disease, myositis, periodontitis, psoriasis, rheumatoid arthritis (RA), scleroderma, sinusitis, Sjogren's syndrome (SS), systemic lupus erythematosus (SLE), tuberculosis, and vasculitis.

In some embodiments, the target is BAFF, and the autoimmune or inflammatory condition is systemic lupus erythematosus (SLE) or antiphospholipid (APL) syndrome. In some embodiments, the target is BAFF, and the autoimmune or inflammatory condition is rheumatoid arthritis (RA), multiple sclerosis (MS), or IgA-nephropathy.

In some embodiments, the target is BAFF Receptor, and the autoimmune or inflammatory condition is systemic lupus erythematosus (SLE) or antiphospholipid (APL) syndrome. In some embodiments, the target is BAFF Receptor, and the autoimmune or inflammatory condition is rheumatoid arthritis (RA), multiple sclerosis (MS), or IgA-nephropathy.

In some embodiments, the target is CD80, and the autoimmune or inflammatory condition is rheumatoid arthritis (RA) or kidney transplant. In some embodiments, the target is CD80, and the autoimmune or inflammatory condition is psoriatic arthritis (PsA), ankylosing spondylitis (AS), or systemic lupus erythematosus (SLE).

In some embodiments, the target is CD86, and the autoimmune or inflammatory condition is rheumatoid arthritis (RA) or kidney transplant. In some embodiments, the target is CD86, and the autoimmune or inflammatory condition is psoriatic arthritis (PsA), ankylosing spondylitis (AS), or systemic lupus erythematosus (SLE).

In some embodiments, the target is CD40, and the autoimmune or inflammatory condition is Sjogren's syndrome (SS), myasthenia gravis (MG), or kidney transplant. In some embodiments, the target is CD40, and the autoimmune or inflammatory condition is systemic lupus erythematosus (SLE).

In some embodiments, the target is CD40 ligand, and the autoimmune or inflammatory condition is Sjogren's syndrome (SS), myasthenia gravis (MG), or kidney transplant. In some embodiments, the target is CD40 ligand, and the autoimmune or inflammatory condition is systemic lupus erythematosus (SLE).

In some embodiments, a pharmaceutical composition comprising an effective amount of a conjugate of the present disclosure is for use in a method of treating or preventing a disease or condition in a subject described herein.

In some embodiments, a use of an effective amount of a conjugate of the present disclosure is in the manufacture of a medicament in a method of treating or preventing a disease or condition in a subject described herein.

In some embodiments, an effective amount of a conjugate of the present disclosure is for use in a method of treating or preventing a disease or condition in a subject described herein.

### VII. Examples

Exemplary chemical entities useful in methods of the description are described by reference to illustrative synthetic schemes for their general preparation below and the specific examples that follow. Artisans recognize that, to obtain the various compounds herein, starting materials may be suitably selected so that the ultimately desired substituents can be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, or the like. Suitable protecting groups for amino, amidino and guanidino include t-butoxycarbonyl, benzyloxycarbonyl, or the like. Suitable protecting groups for mercapto include —C(O)—R″ (where R″ is alkyl, aryl or arylalkyl), p-methoxybenzyl, trityl or the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

Furthermore, all compounds of this disclosure which exist in free base or acid form can be converted to their salts by treatment with the appropriate inorganic or organic base or acid by methods known to one skilled in the art. Salts of the compounds of this disclosure can be converted to their free base or acid form by standard techniques.

The following Examples illustrate exemplary methods of making compounds and conjugates of this disclosure. It is understood that one skilled in the art may be able to make these compounds by similar methods or by combining other methods known to one skilled in the art. It is also understood that one skilled in the art would be able to make, in a similar manner as described below, other compounds and conjugates not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure. The compound 4-((S)-2-((S)-2-amino-3-methylbutanamido)-5-ureidopentanamido)benzyl 3-(2-amino-4-(dipropylcarbamoyl)-3H-benzo[b]azepine-8-carboxamido)-7,8-dihydro-1,6-naphthyridine-6(5H)-carboxylate can be synthesized according to U.S. Pat. No. 10,239,862, the synthetic procedure for this compound is hereby incorporated by reference.

### Abbreviations

The examples described herein use materials, including but not limited to, those described by the following abbreviations known to those skilled in the art:

| | |
|---|---|
| g | grams |
| eq | equivalents |
| mmol | millimoles |
| mL | milliliters |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| ° C. | degrees Celsius |
| N | normal |
| J | coupling constant |
| DMSO-d₆ | deuterated dimethyl sulfoxide |
| EtOH | ethanol |
| DIPEA | N,N-diisopropylethylamine |
| min | minutes |
| hr | hours |
| HPLC | high performance liquid chromatography |
| TLC | thin layer chromatography |
| M | molar |
| MS | mass spectrum |
| m/z | mass-to-charge ratio |
| DMAP | 4-(dimethylamino)pyridine |
| μM | micromolar |
| IC₅₀ | half maximal inhibitory concentration |
| Pd(dppf)Cl₂ | [1,1′-bis(diphenylphosphino)ferrocene]- |
| | dichloropalladium(II) |
| DCM | dichloromethane |
| DMF | N,N-dimethylformamide |
| EtOAc or EA | ethyl acetate |
| NMP | N-methylpyrrolidone |
| TfOH | trifluoromethanesulfonic acid |
| TFA | trifluoroacetic acid |
| THF | tetrahydrofuran |

### Example 1. Preparation of (6aR,6bS,7S,8aS,8bS,10R,11aR,12aS,12bS)-7-hydroxy-8b-(2-hydroxyacetyl)-6a,8a-dimethyl-10-(4-(4-methyl-1H-1,2,3-triazol-5-yl)phenyl)-1,2,6a,6b,7,8,8a,8b,11a,12,12a,12b-dodecahydro-4H-naphtho[2′,1′:4,5]indeno[1,2-d][1,3]dioxol-4-one

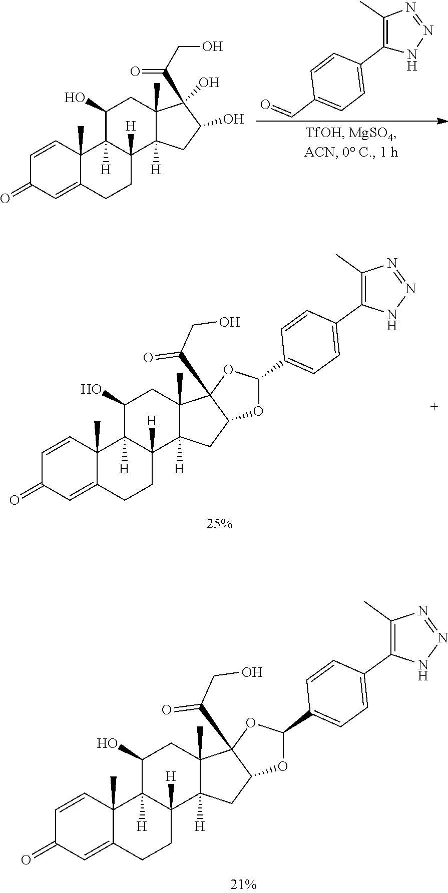

To a solution of 4-(4-methyl-1H-1,2,3-triazol-5-yl)benzaldehyde (20.00 mg, 0.10 mmol, 1.00 equiv) in acetonitrile (1.00 mL) with an inert atmosphere of nitrogen, were added (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (20.11 mg, 0.10 mmol, 1.00 equiv), magnesium sulfate (22.50 mg, 0.35 mmol, 3.50 equiv). The reaction mixture was stirred for 30 min at 0° C., under nitrogen atmosphere followed by the addition of trifluoromethanesulfonic acid (38.48 mg, 0.48 mmol, 4.8 equiv) dropwise at 0° C. The reaction was made alkalinity using sodium bicarbonate and extracted with 3×500 mL of dichloromethane and the organic layers were combined, washed with 3×500 mL of saturated sodium chloride solution, dried over anhydrous sodium sulfate. The concentrated product was purified by Flash-Prep-HPLC with the following conditions (IntelFlash-1): Column, C₁₈ silica gel; mobile phase, water (with 5.0 mmol/L ammonium bicarbonate) and acetonitrile (5.0% acetonitrile up to 100.0% in 30 min); Detector, UV 254 nm. 15.4 mg (25.73%) of (6aR,6bS,7S,8aS,8bS,10R,11aR,12aS,12bS)-7-hydroxy-8b-(2-hydroxyacetyl)-6a,8a-dimethyl-10-(4-(4-methyl-1H-1,2,3-triazol-5-yl)phenyl)-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]+(ESI): 546.25; ¹H NMR (400 MHz, Methanol-d₄) δ: 1.00 (s, 3H), 1.15-1.20 (m, 2H), 1.50 (s, 3H), 1.83-1.91 (m, 4H), 1.99-2.03 (m, 1H), 2.14-2.16 (m, 1H), 2.26-2.28 (m, 1H), 2.37-2.39 (m, 1H), 2.47 (s, 3H), 2.63-2.66 (m, 1H), 4.32-4.43 (m, 2H), 4.63-4.68 (m, 1H), 5.08-5.09 (m, 1H), 5.52 (s, 1H), 6.01 (s, 1H), 6.22-6.25 (m, 1H), 7.42-7.45 (m, 1H), 7.55-7.57 (m, 2H), 7.70-7.72 (m, 2H).

13.0 mg (21.72%) of (6aR,6bS,7S,8aS,8bS,10S,11aR,12aS,12bS)-7-hydroxy-8b-(2-hydroxyacetyl)-6a,8a-dimethyl-10-(4-(4-methyl-1H-1,2,3-triazol-5-yl)phenyl)-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]+ (ESI): 546.20; ¹H NMR (400 MHz, Methanol-d₄) δ: 1.02 (s, 3H), 1.13-1.16 (m, 2H), 1.51 (s, 3H), 1.76-1.82 (m, 2H), 1.83-1.94 (m, 2H), 2.03-2.07 (m, 1H), 2.16-2.24 (m, 2H), 2.42-2.43 (m, 1H), 2.48 (s, 3H), 2.67-2.69 (m, 1H), 4.11-4.16 (m, 1H), 4.32-4.44 (m, 2H), 5.42-5.44 (m, 1H), 6.03 (s, 1H), 6.19 (s, 1H), 6.25-6.28 (m, 1H), 7.42-7.48 (m, 3H), 7.60-7.68 (m, 2H).

### Preparation of 4-(prop-1-yn-1-yl)benzaldehyde

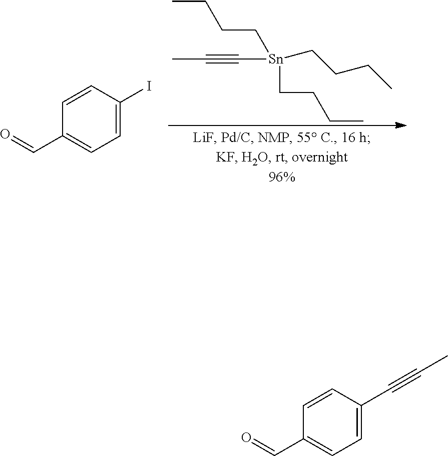

To a solution of 4-iodobenzaldehyde (500.00 mg, 2.16 mmol, 1.00 equiv) in N-methyl-2-pyrrolidone (5.00 mL) with an inert atmosphere of nitrogen, were added tributyl(prop-1-yn-1-yl)stannane (780.16 mg, 2.37 mmol, 1.10 equiv), Lithium fluoride (111.80 mg, 4.31 mmol, 2.00 equiv) and Palladium on Carbon (45.87 mg, 0.43 mmol, 0.20 equiv). The resulting solution was stirred for 16 h at 55° C. The mixture was allowed to cool down to 25° C. To the mixture at room temperature was added a saturated aqueous KF solution (10 mL), and the mixture was stirred overnight. The resulting mixture was diluted with water (100 mL). The resulting mixture was extracted with dichloromethane (3×100 mL). The combined organic layers were washed with sodium chloride, dried over anhydrous sodium sulfate. After filtration, the filtrate was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 30 min); Detector, UV 254 nm. 300.00 mg (96.56%) of 4-(prop-1-yn-1-yl)benzaldehyde was obtained as a yellow solid: MS m/z [M+H]⁺ (ESI): 144.06.

### Preparation of 4-(4-methyl-1H-1,2,3-triazol-5-yl)benzaldehyde

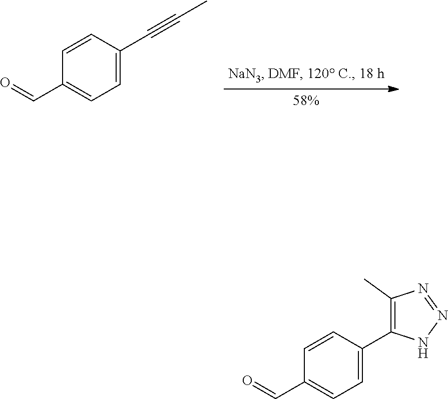

To a solution of 4-(prop-1-yn-1-yl)benzaldehyde (200.00 mg, 1.39 mmol, 1.00 equiv) in Dimethyl Formamide (8.00 mL) with an inert atmosphere of nitrogen, were added sodium azide (135.28 mg, 2.08 mmol, 1.50 equiv). The reaction mixture was stirred for 18 h at 120° C. The crude product was purified by Flash-Prep-HPLC with the following conditions (IntelFlash-1): Column, C₁₈ silica gel; mobile phase, water and acetonitrile (10.0% acetonitrile up to 100.0% in 30 min); Detector, UV 254 nm. 152.00 mg (58.53%) of 4-(4-methyl-1H-1,2,3-triazol-5-yl)benzaldehyde was obtained as a yellow solid: MS m/z [M+H]+ (ESI): 187.07.

### Example 2. Preparation of (6aR,6bS,7S,8aS,8bS,10R,11aR,12aS,12bS)-10-(4-((1H-1,2,3-triazol-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

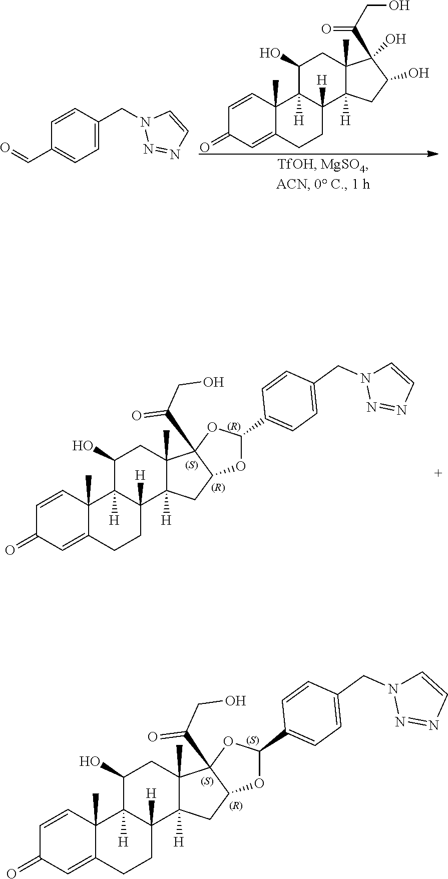

To a solution of 4-((1H-1,2,3-triazol-1-yl)methyl)benzaldehyde (120.0 mg, 0.6 mmol, 1.0 equiv.) in acetonitrile (5.0 mL) with an inert atmosphere of nitrogen, was added (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (241.3 mg, 0.6 mmol, 1.0 equiv.), magnesium sulfate (270.0 mg, 2.2 mmol, 3.5 equiv) for 3 min at 0° C. followed by the addition of trifluoromethanesulfonic acid (384.8 mg, 2.6 mmol, 4.0 equiv.) dropwise at 0° C. The resulting solution was stirred for 1 h at 0° C., adjusted pH to 7 with saturated sodium bicarbonate solution (1.0 mL). The mixture was diluted with 5 mL of water and extracted with 3×50 mL of dichloromethane and the organic layers were combined, washed with 3×50 mL of saturated sodium chloride, dried over anhydrous sodium sulfate, filtered, and concentrated under reduced pressure. 30.4 mg (8.3%) of (6aR,6bS,7S,8aS,8bS,10R,11aR,12aS,12bS)-10-(4-((1H-1,2,3-triazol-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 546.20; ¹H NMR (400 MHz, DMSO-d₆) δ: 0.86 (s, 3H), 0.99-1.03 (m, 2H), 1.39 (s, 3H), 1.64-1.77 (m, 5H), 2.00-2.15 (m, 2H), 2.31-2.32 (m, 1H), 2.52-2.54 (m, 1H), 4.15-4.19 (m, 1H), 4.21-4.29 (m, 1H), 4.48-4.55 (m, 1H), 4.78-4.79 (m, 1H), 4.93-4.94 (m, 1H), 5.09-5.12 (m, 1H), 5.44 (s, 1H), 5.62 (s, 2H), 5.93 (s, 1H), 6.15-6.18 (m, 1H), 7.29-7.32 (m, 3H), 7.46-7.48 (m, 2H), 7.74 (s, 1H), 8.17 (s, 1H).

16.0 mg (4.5%) of (6aR,6bS,7S,8aS,8bS,10S,11aR,12aS,12bS)-10-(4-((1H-1,2,3-triazol-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 546.20; ¹H NMR (400 MHz, DMSO-d₆) δ: 0.89 (s, 3H), 1.03-1.07 (m, 2H), 1.39 (s, 3H), 1.72-1.87 (m, 5H), 1.99-2.08 (m, 2H), 2.32-2.33 (m, 1H), 2.53-2.54 (m, 1H), 3.98-4.04 (m, 1H), 4.21-4.30 (m, 2H), 4.78-4.79 (m, 1H), 4.99-5.03 (m, 1H), 5.29-5.31 (m, 1H), 5.63 (s, 2H), 5.94 (s, 1H), 6.09 (s, 1H), 6.16-6.19 (m, 1H), 7.28-7.33 (m, 5H), 7.74 (s, 1H), 8.17 (s, 1H).

### Preparation 4-((1H-1,2,3-triazol-1-yl)methyl)benzaldehyde

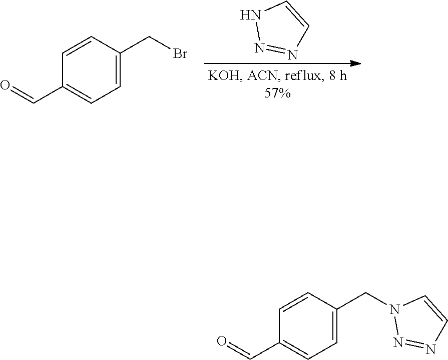 [SMILES: O=Cc1ccc(CBr)cc1 ; c1c[nH]nn1 ; O=Cc1ccc(Cn2ccnn2)cc1]

To a solution of 4-(bromomethyl)benzaldehyde (150.0 mg, 0.8 mmol, 1 equiv.) in acetonitrile (5.0 mL) with an inert atmosphere of nitrogen, was added 1,2,3-triazole (52.1 mg, 0.8 mmol, 1 equiv.), potassium hydroxide (63.4 mg, 1.1 mmol, 1.5 equiv.) at 25° C. The resulting mixture was stirred for 8 h at 90° C. The mixture was allowed to cool down to room temperature, diluted with 50 mL of water and extracted with 3×100 mL of dichloromethane and the organic layers were combined, washed with 3×100 mL of saturated sodium chloride, dried over anhydrous sodium sulfate, filtered, and concentrated under reduced pressure. The crude product was purified by Flash-Prep-HPLC with the following conditions (IntelFlash-1): Column, C₁₈ silica gel; mobile phase, water, and acetonitrile (10.0% acetonitrile up to 100.0% in 20 min); Detector, UV 254 nm. 80 mg (57%) of 4-((1H-1,2,3-triazol-1-yl)methyl)benzaldehyde was obtained as a white solid: MS m/z [M+H]⁺ (ESI): 188.20.

### Example 3. Preparation of (6aR,6bS,7S,8aS,8bS,10R,11aR,12aS,12bS)-10-(4-((1H-1,2,3-triazol-4-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2d][1,3]dioxol-4-one

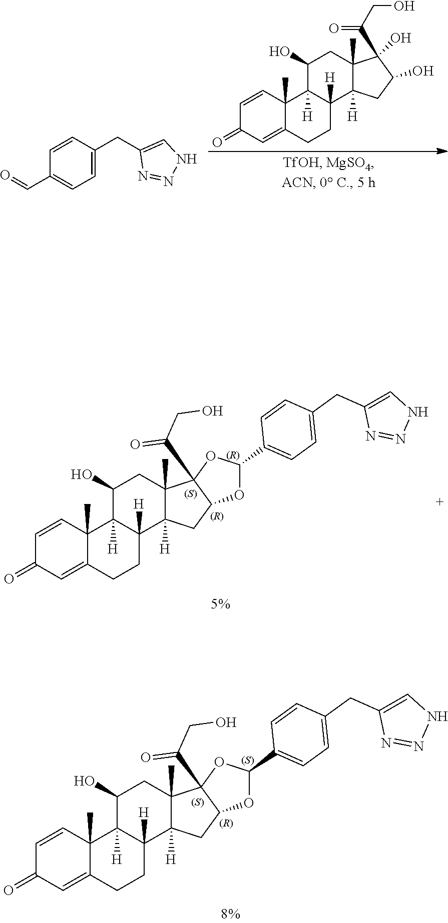

To a stirred solution of 4-((1H-1,2,3-triazol-4-yl)methyl)benzaldehyde (70.0 mg, 0.37 mmol, 1.0 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (140.7 mg, 0.37 mmol, 1.0 equiv) in acetonitrile were added magnesium sulfate (157.5 mg, 1.20 mmol, 3.5 equiv) and trifluoromethanesulfonic acid (168.3 mg, 0.96 mmol, 3.0 equiv) dropwise at 0° C. The residue was purified by reverse flash chromatography with the following conditions: column, silica gel; mobile phase, acetonitrile in water, 10% to 100% gradient in 20 min; detector, UV 254 nm. This resulted in (6aR,6bS,7S,8aS,8bS,10R,11aR,12aS,12bS)-10-(4-((1H-1,2,3-triazol-4-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (11.9 mg, 5.81%) as a white solid: MS m/z [M+H]⁺ (ESI): 546.25; ¹H NMR (400 MHz, Methanol-d₄) δ: 0.98 (s, 3H), 1.02-1.06 (m, 2H), 1.49 (s, 3H), 1.70-1.88 (m, 4H), 1.94-1.98 (m, 1H), 2.13-2.24 (m, 1H), 2.37-2.40 (m, 2H), 2.65-2.66 (m, 1H), 4.07 (s, 2H), 4.29-4.42 (m, 2H), 4.59-4.64 (m, 1H), 5.04-5.05 (m, 1H), 5.45 (s, 1H), 6.01 (s, 1H), 6.23-6.26 (m, 1H), 7.25-7.27 (m, 2H), 7.38-7.42 (m, 3H), 7.45 (s, 1H).

6.2 mg (8.99% yield) of (6aR,6bS,7S,8aS,8bS,10S,11aR,12aS,12bS)-10-(4-((1H-1,2,3-triazol-4-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 546.25; ¹H NMR (400 MHz, Methanol-d₄) δ: 0.99 (s, 3H), 1.11-1.14 (m, 2H), 1.49 (s, 3H), 1.74-1.89 (m, 4H), 1.91-1.99 (m, 1H), 2.14-2.22 (m, 2H), 2.38-2.42 (m, 1H), 2.66-2.68 (m, 1H), 4.07-4.11 (m, 3H), 4.26-4.31 (m, 1H), 4.42 (s, 1H), 5.38-5.39 (m, 1H), 6.02 (s, 1H), 6.12 (s, 1H), 6.24-6.27 (m, 1H), 7.21-7.24 (m, 4H), 7.45 (d, J=10.0 Hz, 1H), 7.55 (s, 1H).

### Preparation of 4-(3-(trimethylsilyl)prop-2-yn-1-yl)benzaldehyde

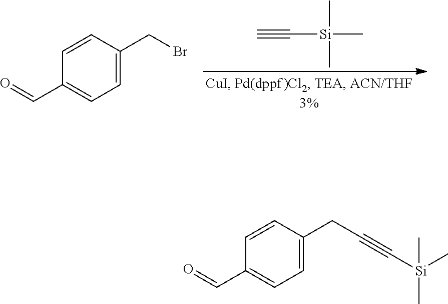

A solution of 4-(bromomethyl)benzaldehyde (1.0 g, 5.02 mmol, 1.0 equiv) and trimethylsilylacetylene (2.4 g, 25.10 mmol, 5.0 equiv), cuprous iodide (95.7 mg, 0.500 mmol, 0.1 equiv), [1,1′-Bis(diphenylphosphino)ferrocene]dichloropalladium(II) (919.0 mg, 1.26 mmol, 0.25 equiv), trimethylamine (1.5 g, 15.06 mmol, 3.0 equiv) in acetonitrile (10 mL) was stirred for 20 h at room temperature under nitrogen atmosphere. The residue was purified by reverse flash chromatography with the following conditions: column, C₁₈ silica gel; mobile phase, acetonitrile in water, 10% to 100% gradient in 20 min; detector, UV 254 nm. This resulted in 4-(3-(trimethylsilyl)prop-2-yn-1-yl)benzaldehyde (300 mg, 3.8%) as a white solid.

### Preparation of 4-((5-(trimethylsilyl)-1H-1,2,3-triazol-4 yl)methyl)benzaldehyde

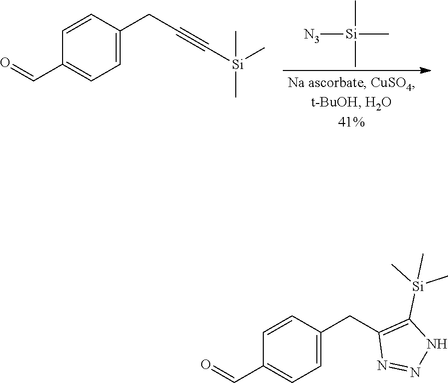

A solution of 4-(3-(trimethylsilyl)prop-2-yn-1-yl)benzaldehyde (300.0 mg, 1.38 mmol, 1.0 equiv) in tertiary butyl alcohol:water (3 mL, V:V=1:1) was treated with azidotrimethylsilane (479.40 mg, 4.14 mmol, 3 equiv) for 1 min at 25° C. followed by the addition of sodium ascorbate (247.33 mg, 1.242 mmol, 0.9 equiv) in portions at 25° C. To the above mixture was added pentahydrate copper sulphate (1.9 g, 12.22 mmol, 0.4 equiv) in portions over 1 min at room temperature. The resulting mixture was stirred for additional 1 day at room temperature. The resulting solution was purified by Flash-Prep-HPLC with the following conditions (IntelFlash-1): Column, C₁₈ silica gel; mobile phase, water (0.05% ammonium bicarbonate) and acetonitrile (10% acetonitrile up to 100% in 20 min and hold 100% for 5 min); Detector, UV 254 nm. 150 mg (41.7% yield) of 4-((5-(trimethylsilyl)-1H-1,2,3-triazol-4-yl)methyl)benzaldehyde was obtained as an off-white solid.

### Preparation of 4-((5-(trimethylsilyl)-1H-1,2,3-triazol-4-yl)methyl)benzaldehyde

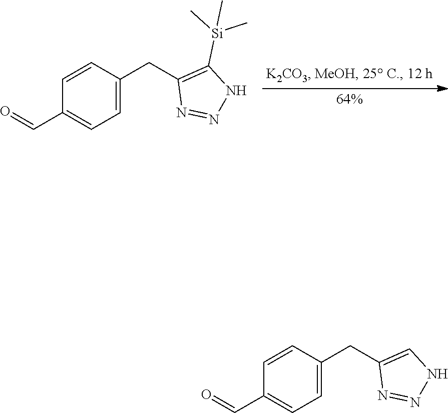 [SMILES: C[Si](C)(C)c1[nH]nnc1Cc1ccc(C=O)cc1 ; O=Cc1ccc(Cc2c[nH]nn2)cc1]

To a stirred solution of 4-((5-(trimethylsilyl)-1H-1,2,3-triazol-4-yl)methyl)benzaldehyde (150.0 mg, 0.57 mmol, 1.0 equiv) and potassium carbonate (119.8 mg, 0.86 mmol, 1.5 equiv) in methanol (2.0 mL) at 25° C. The resulting mixture was stirred for 12 h at 25° C. The aqueous layer was extracted with methylene chloride (3×10 mL). The residue was purified by reverse flash chromatography with the following conditions: column, silica gel; mobile phase, acetonitrile in water, 10% to 100% gradient in 20 min; detector, UV 254 nm. This resulted in 4-((1H-1,2,3-triazol-4-yl)methyl)benzaldehyde (70 mg, 64.6%) as a white solid.

### Example 4. Preparation of (6aR,6bS,7S,8aS,8bS,10R,11aR,12aS,12bS)-7-hydroxy-8b-(2-hydroxyacetyl)-6a,8a-dimethyl-10-(1-methyl-1H-pyrazol-5-yl)-1,2,6a,6b,7,8,8a,8b,11a,12,12a,12b-dodecahydro-4H-naphtho[2′,1′:4,5]indeno[1,2-d][1,3]dioxol-4-one

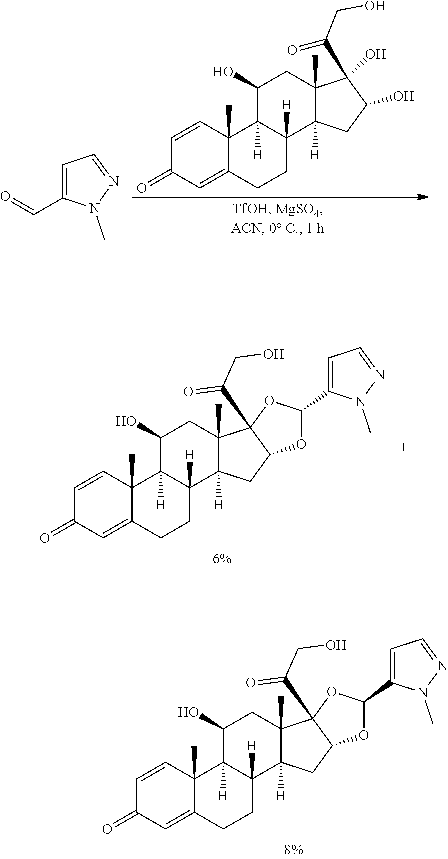

To a solution of 2-methylpyrazole-3-carbaldehyde (50.0 mg, 0.5 mmol, 1.0 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (170.9 mg, 0.5 mmol, 1.0 equiv) with an inert atmosphere of nitrogen, was added magnesium sulfate (163.9 mg, 1.4 mmol, 3.0 equiv), trifluoromethanesulfonic acid (204.4 mg, 1.4 mmol, 3.0 equiv) in order at −20° C. The resulting solution was stirred for 1 h at 0° C., adjusted pH value to 7 with saturated sodium bicarbonate solution. The aqueous layer was extracted with 3×35 mL of dichloromethane, dried over anhydrous sodium sulfate, filtered, and concentrated under reduced pressure. The crude product was purified by Flash-Prep-HPLC with the following conditions (IntelFlash-1): Column, C₁₈ silica gel; mobile phase, acetonitrile in water, 10% to 100% gradient in 15 min; detector, UV 254 nm). 13.1 mg (6%) of (6aR,6bS,7S,8aS,8bS,10R,11aR,12aS,12bS)-7-hydroxy-8b-(2-hydroxyacetyl)-6a,8a-dimethyl-10-(1-methyl-1H-pyrazol-5-yl)-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]+ (ESI): 469.15; ¹H NMR (400 MHz, Methanol-d₄) δ: 1.00-1.10 (m, 5H), 1.50 (s, 3H), 1.60-1.70 (m, 2H), 1.82-1.85 (m, 2H), 1.94-1.98 (m, 1H), 2.10-2.14 (m, 1H), 2.20-2.30 (m, 1H), 2.35-2.39 (m, 1H), 2.64-2.65 (m, 1H), 3.86 (s, 3H), 4.32-4.37 (m, 1H), 4.43-4.44 (m, 1H), 4.61-4.66 (m, 1H), 5.09-5.11 (m, 1H), 5.75 (s, 1H), 5.99 (s, 1H), 6.23-6.26 (m, 1H), 6.39 (s, 1H), 7.38-7.45 (m, 2H).

18.6 mg (8%) of (6aR,6bS,7S,8aS,8bS,10S,11aR,12aS,12bS)-7-hydroxy-8b-(2-hydroxyacetyl)-6a,8a-dimethyl-10-(1-methyl-1H-pyrazol-5-yl)-1,2,6a,6b,7,8,8a,8b,11a,12,12a,12b-dodecahydro-4H-naphtho[2′,1′:4,5]indeno[1,2-d][1,3]dioxol-4-one was obtained as white solid: MS m/z [M+H]+ (ESI): 469.15; ¹H NMR (400 MHz, Methanol-d₄) δ: 0.98 (s, 3H), 1.11-1.15 (m, 1H), 1.20-1.30 (m, 1H), 1.49 (s, 3H), 1.73-1.79 (m, 2H), 1.86-1.89 (m, 2H), 2.01-2.05 (m, 1H), 2.13-2.24 (m, 2H), 2.38-2.42 (m, 1H), 2.67-2.68 (m, 1H), 3.82 (s, 3H), 3.98-4.03 (m, 1H), 4.11-4.16 (m, 1H), 4.42-4.46 (m, 1H), 5.37-5.39 (m, 1H), 6.02 (s, 1H), 6.18 (s, 1H), 6.24-6.27 (m, 1H), 6.38 (s, 1H), 7.33 (s, 1H), 7.40-7.45 (m, 1H).

### Example 5. Preparation of (6aR,6bS,7S,8aS,8bS,10R,11aR,12aS,12bS)-7-hydroxy-8b-(2-hydroxyacetyl)-6a,8a-dimethyl-10-(1-methyl-1H-pyrazol-4-yl)-1,2,6a,6b,7,8,8a,8b,11a,12,12a,12b-dodecahydro-4H-naphtho[2′,1′:4,5]indeno[1,2-d][1,3]dioxol-4-one

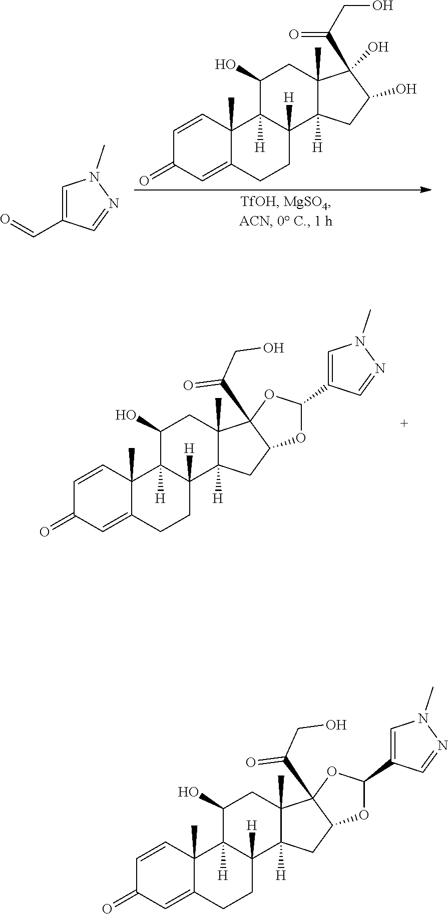

To a solution of 1-methylpyrazole-4-carbaldehyde (50 mg, 0.5 mmol, 1.0 equiv) in acetonitrile (5.0 mL) with an inert atmosphere of nitrogen, was added (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (170.9 mg, 0.5 mmol, 1.0 equiv), magnesium sulfate (190.9 mg, 1.6 mmol, 3.5 equiv) for 3 min at 0° C. followed by the addition of trifluoromethanesulfonic acid (238.51 mg, 1.589 mmol, 3.5 equiv) dropwise at 0° C. The resulting solution was stirred for 1 h at 0° C., adjusted pH value to 7 with saturated sodium bicarbonate solution (1.0 mL). The mixture was diluted with 5 mL of water and extracted with 3×50 mL of dichloromethane. The organic layers were combined, washed with 3×50 mL of saturated sodium chloride, dried over anhydrous sodium sulfate, filtered, and concentrated under reduced pressure. The crude product was purified by Flash with the following conditions: Column, C₁₈ silica gel; mobile phase, water and acetonitrile (5.0% acetonitrile up to 100.0% in 20 min); Detector, UV 254 nm. 21 mg (10%) of (6aR,6bS,7S,8aS,8bS,10R,11aR,12aS,12bS)-7-hydroxy-8b-(2-hydroxyacetyl)-6a,8a-dimethyl-10 (1-methyl-1H-pyrazol-4-yl)-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 469.25; ¹H NMR (400 MHz, Methanol-d₄) δ: 0.96 (s, 3H), 1.09-1.19 (m, 2H), 1.49 (s, 3H), 1.67-1.82 (m, 4H), 1.94-1.98 (m, 1H), 2.12-2.15 (m, 1H), 2.24-2.26 (m, 1H), 2.37-2.40 (m, 1H), 2.64-2.67 (m, 1H), 3.85 (s, 3H), 4.27-4.32 (m, 1H), 4.42-4.43 (m, 1H), 4.57-4.62 (m, 1H), 4.98-4.99 (m, 1H), 5.53 (s, 1H), 6.02 (s, 1H), 6.23-6.26 (m, 1H), 7.44-7.50 (m, 2H), 7.70 (s, 1H).

15 mg (7%) of (6aR,6bS,7S,8aS,8bS,10S,11aR,12aS,12bS)-7-hydroxy-8b-(2-hydroxyacetyl)-6a,8a-dimethyl-10-(1-methyl-1H-pyrazol-4-yl)-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 469.25; ¹H NMR (400 MHz, Methanol-d₄) δ: 0.99 (s, 3H), 1.08-1.11 (m, 2H), 1.49 (s, 3H), 1.70-1.87 (m, 4H), 1.97-2.02 (m, 1H), 2.13-2.21 (m, 2H), 2.37-2.41 (m, 1H), 2.66-2.67 (m, 1H), 3.83 (s, 3H), 4.10-4.15 (m, 1H), 4.37-4.42 (m, 2H), 5.29-5.31 (m, 1H), 6.02 (s, 1H), 6.19 (s, 1H), 6.24-6.27 (m, 1H), 7.38 (s, 1H), 7.44-7.47 (m, 1H), 7.55 (s, 1H).

### Example 6. Preparation of (6aR,6bS,7S,8aS,8bS,10R,11aR,12aS,12bS)-10-(benzo[d][1,3]dioxol-5-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

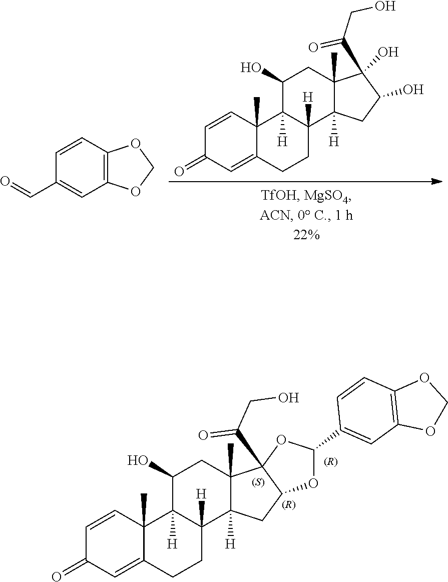

To a solution of benzo[d][1,3]dioxole-5-carbaldehyde (40.0 mg, 0.13 mmol, 1.0 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (150.4 mg, 0.20 mmol, 1.5 equiv) in acetonitrile (0.4 mL) with an inert atmosphere of nitrogen, were added dropwise trifluoromethanesulfonic acid (100.0 mg, 0.33 mmol, 2.5 equiv) and magnesium sulfate (96.2 mg, 0.40 mmol, 3.0 equiv) in portions at 0° C. The resulting mixture was stirred for 1 h at 0° C. The resulting solution was purified by Flash with the following conditions (column, C₁₈ silica gel; mobile phase, water (0.1% Ammonium hydroxide+10 mmol/L ammonium bicarbonate) and acetonitrile (10.0% acetonitrile up to 100.0% in 20 min); Detector, UV 254 nm. 30 mg (22%) of (6aR,6bS,7S,8aS,8bS,10R,11aR,12aS,12bS)-10-(benzo[d][1,3]dioxol-5-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 509.20; ¹H NMR (400 MHz, Methanol-d₄) δ: 0.98 (s, 3H), 1.04-1.07 (m, 2H), 1.49 (s, 3H), 1.71-1.87 (m, 4H), 1.94-1.98 (m, 1H), 2.15-2.30 (m, 2H), 2.35-2.40 (m, 1H), 2.60-2.70 (m, 1H), 4.28-4.33 (m, 1H), 4.42 (s, 1H), 4.58-4.63 (m, 1H), 5.02-5.03 (m, 1H), 5.38 (s, 1H), 5.94-6.03 (m, 3H), 6.24-6.27 (m, 1H), 6.78-6.80 (m, 1H), 6.91-6.93 (m, 2H), 7.40-7.43 (m, 1H).

### Preparation of benzo[d][1,3]dioxole-5-carbaldehyde

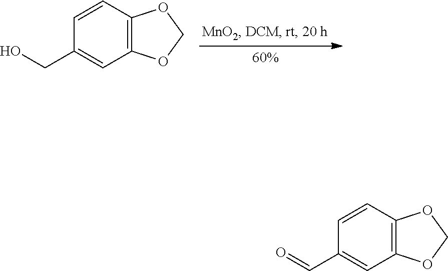 [SMILES: OCc1ccc2c(c1)OCO2 ; O=Cc1ccc2c(c1)OCO2]

To a solution of 1,3-benzodioxole-5-methanol (500.0 mg, 0.33 mmol, 1.0 equiv) and manganese dioxide (2850.7 mg, 3.3 mmol, 10.0 equiv) in dichloromethane (5 mL) with an inert atmosphere of nitrogen, were stirred for 20 h at room temperature. The precipitated solids were collected by filtration and washed with dichloromethane (5×10 mL). The crude product was purified by Flash-Prep-HPLC with the following conditions (IntelFlash-1): Column, C₁₈ silica gel; mobile phase, water (5 mmol/L ammonium bicarbonate) and acetonitrile (10.0% acetonitrile up to 100.0% in 20 min); Detector, UV 254 nm. 300.0 mg (60.81%) of benzo[d][1,3]dioxole-5-carbaldehyde was obtained as a white solid: MS m/z [M+H]⁺ (ESI): 151.03.

### Example 7. Preparation of (6aR,6bS,7S,8aS,8bS,10R,11aR,12aS,12bS)-10-(2,3-dihydrobenzo[b][1,4]dioxin-6-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

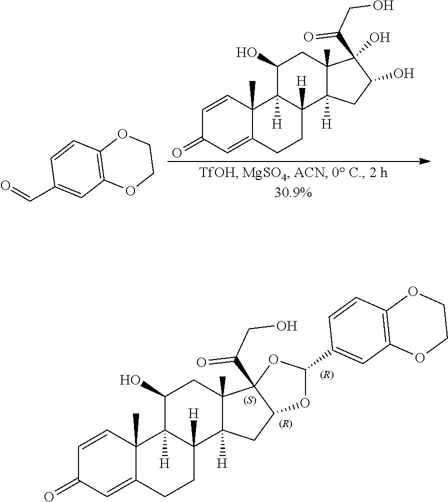

To a solution of 2,3-dihydro-1,4-benzodioxine-6-carbaldehyde (20.0 mg, 0.12 mmol, 1.0 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (68.8 mg, 0.18 mmol, 1.5 equiv) in acetonitrile (0.2 mL) with an inert atmosphere of nitrogen, were added dropwise trifluoromethanesulfonic acid (45.7 mg, 0.31 mmol, 2.5 equiv) and magnesium sulfate (44.0 mg, 0.37 mmol, 3.0 equiv) in portions at 0° C. The resulting mixture was stirred for 2 h at 0° C. The resulting solution was purified by Flash with the following conditions (column, C₁₈ silica gel; mobile phase, water, and acetonitrile (10.0% acetonitrile up to 100.0% in 20 min); Detector, UV 254 nm. 19.9 mg (30.94%) of (6aR,6bS,7S,8aS,8bS,10R,11aR,12aS,12bS)-10-(2,3-dihydrobenzo[b][1,4]dioxin-6-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M−H]⁻ (ESI): 523.20; ¹H NMR (400 MHz, Methanol-d₄) δ: 0.97 (s, 3H), 1.03-1.06 (m, 1H), 1.13-1.15 (m, 1H), 1.49 (s, 3H), 1.69-1.87 (m, 4H), 1.93-1.98 (m, 1H), 2.10-2.30 (m, 2H), 2.35-2.41 (m, 1H), 2.60-2.70 (m, 1H), 4.22 (m, 4H), 4.28-4.42 (m, 1H), 4.43 (s, 1H), 4.57-4.62 (m, 1H), 5.01-5.02 (m, 1H), 5.35 (s, 1H), 6.03 (s, 1H), 6.24-6.26 (m, 1H), 6.79-6.82 (m, 1H), 6.88-6.91 (m, 2H), 7.40-7.43 (m, 1H).

### Example 8. Preparation of (6aS,6bR,7S,8aS,8bS,10R,11aR,12aS,12bS)-6b-fluoro-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one

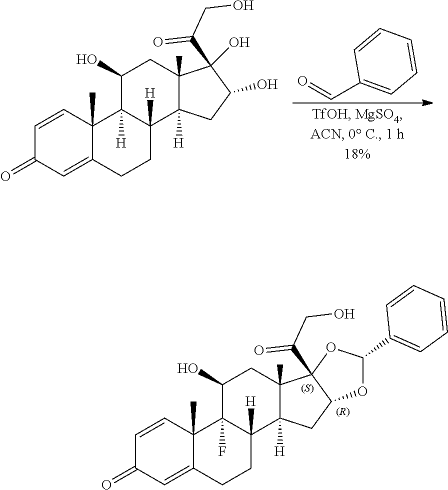

To a solution of triamcinolone (100.0 mg, 0.30 mmol, 1.0 equiv) in acetonitrile (5.0 mL) with an inert atmosphere of nitrogen, was added and benzaldehyde (26.9 mg, 0.30 mmol, 1.0 equiv), magnesium sulfate (106.6 mg, 0.90 mmol, 3.5 equiv) for 3 min at 0° C. followed by the addition of trifluoromethanesulfonic acid (133.2 mg, 0.90 mmol, 3.5 equiv) dropwise at 0° C. The resulting solution was stirred for 1 h at 0° C., adjusted pH value to 7 with saturated sodium bicarbonate solution (1.0 mL). The mixture was diluted with 5 mL of water and extracted with 3×50 mL of dichloromethane. The organic layers were combined, washed with 3×50 mL of saturated sodium chloride, dried over anhydrous sodium sulfate, filtered, and concentrated under reduced pressure. The resulting solution was purified by Flash with the following conditions (column, C₁₈ silica gel; mobile phase, water and acetonitrile (10.0% acetonitrile up to 100.0% in 20 min); Detector, UV 254 nm. 22.4 mg (18%) of (6aS,6bR,7S,8aS,8bS,10R,11aR,12aS,12bS)-6b-fluoro-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 483.35; ¹H NMR (400 MHz, Methanol-d₄) δ: 0.99 (s, 3H), 1.54-1.57 (m, 1H), 1.59 (s, 3H), 1.72-1.79 (m, 3H), 1.93-1.97 (m, 1H), 2.26-2.44 (m, 3H), 2.55-2.70 (m, 1H), 2.74-2.75 (m, 1H), 4.30-4.35 (m, 2H), 4.61-4.86 (m, 1H), 5.05-5.06 (m, 1H), 5.48 (s, 1H), 6.11 (s, 1H), 6.29-6.32 (m, 1H), 7.35-7.39 (m, 4H), 7.43-7.45 (m, 2H).

### Example 9. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one

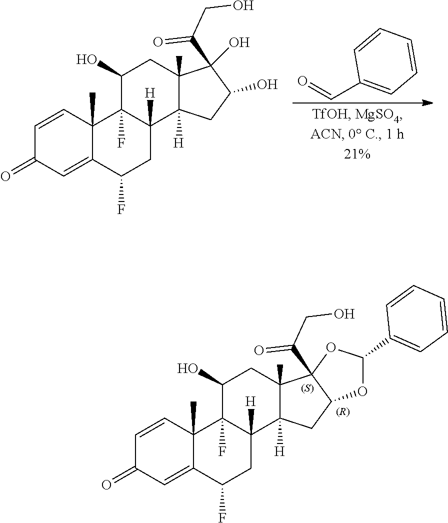

To a solution of fluocinolone (100.0 mg, 0.24 mmol, 1.0 equiv) in acetonitrile (5.0 mL) with an inert atmosphere of nitrogen, was added magnesium sulfate (102.1 mg, 0.80 mmol, 3.5 equiv) and benzaldehyde (25.7 mg, 0.24 mmol, 1.0 equiv) for 3 min at 0° C. followed by the addition of trifluoromethanesulfonic acid (109.2 mg, 0.70 mmol, 3.0 equiv). The resulting solution was stirred for 1 h at 0° C., adjusted pH value to 7 with saturated sodium bicarbonate solution (1.0 mL). The mixture was diluted with 5 mL of water and extracted with 3×50 mL of dichloromethane and the organic layers were combined, washed with 3×50 mL of saturated sodium chloride, dried over anhydrous sodium sulfate, filtered, and concentrated under reduced pressure. The resulting solution was purified by Flash with the following conditions (column, C₁₈ silica gel; mobile phase, water, and acetonitrile (10.0% acetonitrile up to 100.0% in 20 min); Detector, UV 254 nm. 25 mg (21%) of (2S,6aS,6bR,7S,8aS,8bS,10R,11aR,12aS,12bS)-2,6b-difluoro-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 501.20; ¹H NMR (400 MHz, Methanol-d₄) δ: 0.99 (s, 3H), 1.58 (s, 3H), 1.63-1.82 (m, 4H), 2.26-2.29 (m, 1H), 2.36-2.43 (m, 2H), 2.60-2.70 (m, 1H), 4.29-4.36 (m, 2H), 4.62-4.66 (m, 1H), 5.07-5.08 (m, 1H), 5.49 (s, 2H), 6.32-6.35 (m, 2H), 7.31-7.34 (m, 1H), 7.36-7.39 (m, 3H), 7.44-7.46 (m, 2H).

### Example 10. Preparation of (2S,6aS,6bR,7S,8aS,8bS,10R,11aR,12aS,12bS)-2,6b-difluoro-10-(4-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

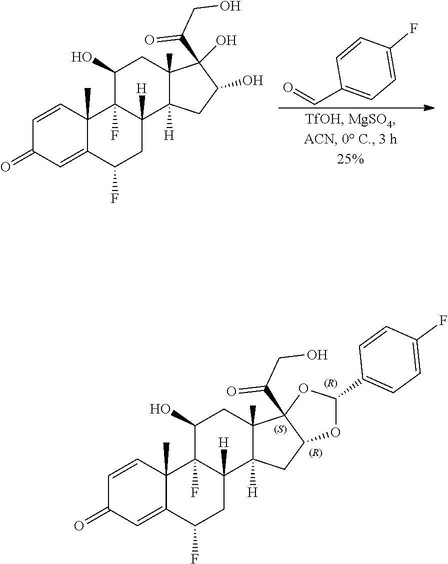

To a solution of fluocinolone (100.0 mg, 0.20 mmol, 1.0 equiv) in acetonitrile (5.0 mL) with an inert atmosphere of nitrogen, was added 4-fluorobenzaldehyde (45.1 mg, 0.40 mmol, 1.5 equiv), magnesium sulfate (102.1 mg, 0.80 mmol, 3.5 equiv) for 3 min at 0° C. followed by the addition of trifluoromethanesulfonic acid (109.2 mg, 0.7 mmol, 3.0 equiv) dropwise at 0° C. The resulting solution was stirred for 3 h at 0° C., adjusted pH value to 7 with saturated sodium bicarbonate solution (1.0 mL). The mixture was diluted with 5 mL of water and extracted with 3×50 mL of dichloromethane. The organic layers were combined, washed with 3×50 mL of saturated sodium chloride, dried over anhydrous sodium sulfate, filtered, and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 36.2 mg (25%) of (2S,6aS,6bR,7S,8aS,8bS,10R,11aR,12aS,12bS)-2,6b-difluoro-10-(4-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 519.15; ¹H NMR (300 MHz, Methanol-d₄) δ: 1.01 (s, 3H), 1.60-1.86 (m, 7H), 2.25-2.39 (m, 1H), 2.42-2.46 (m, 2H), 2.68-2.78 (m, 1H), 4.32-4.38 (m, 2H), 4.66 (d, J=19.5 Hz, 1H), 5.08-5.10 (m, 1H), 5.49-5.67 (m, 2H), 6.34-6.38 (m, 2H), 7.11-7.17 (m, 2H), 7.32-7.48 (m, 1H), 7.49-7.53 (m, 2H).

### Example 11. Preparation of (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

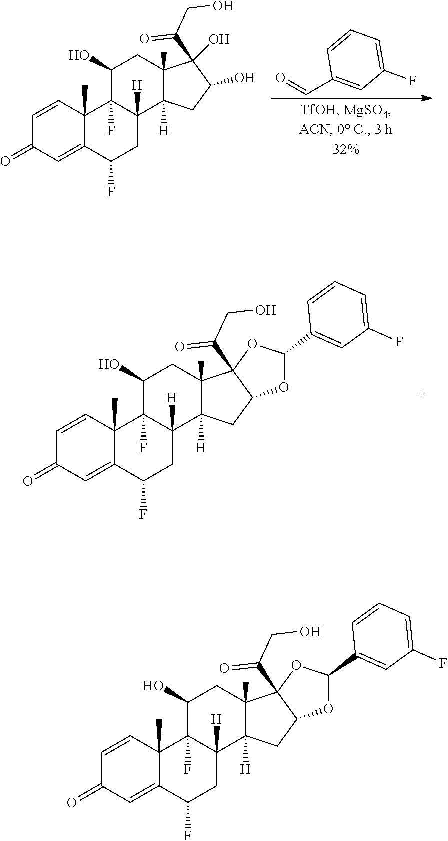

To a solution of fluocinolone (2.0 g, 4.85 mmol, 1.0 equiv.) in acetonitrile (20.0 mL) with an inert atmosphere of nitrogen, was added magnesium sulfate (2.0 g, 16.9 mmol, 3.5 equiv.) and 3-fluorobenzaldehyde (903.0 mg, 7.28 mmol, 1.5 equiv.), followed by the addition of trifluoromethanesulfonic acid (2.2 g, 14.6 mmol, 3.0 equiv.) at 0° C. The resulting solution was stirred for 3 h at 0° C., adjusted pH value to 7 with saturated sodium bicarbonate solution (1.0 mL). The mixture was diluted with 300 mL of water and extracted with 3×500 mL of dichloromethane. The organic layers were combined, washed with 3×500 mL of saturated sodium chloride solution, dried over anhydrous sodium sulfate, filtered, and concentrated under reduced pressure. The crude product (>1 g) was purified by achiral-SFC with the following conditions: Column, Torus 2-PIC Column 4.6×100 mm, 5 μm; mobile phase: isopropyl alcohol (1% 2 mol/L NH₃-methanol) and CO₂; Detector, UV 254 nm. 800 mg (32%) of (2S,6aS,6bR,7S,8aS,8bS,10R,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 519.20; ¹H NMR (400 MHz, Methanol-d₄) δ: 0.99 (s, 3H), 1.28-1.65 (m, 4H), 1.71-1.83 (m, 3H), 2.24-2.39 (m, 3H), 2.66-2.74 (m, 1H), 4.31-4.36 (m, 2H), 4.65 (d, J=19.6 Hz, 1H), 5.08-5.09 (m, 1H), 5.49-5.62 (m, 2H), 6.32-6.35 (m, 2H), 7.09-7.18 (m, 2H), 7.27-7.33 (m, 2H), 7.38-7.41 (m, 1H).

### Example 12. Preparation of (2S,6aS,6bR,7S,8aS,8bS,10R,11aR,12aS,12bS)-10-(3,4-difluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

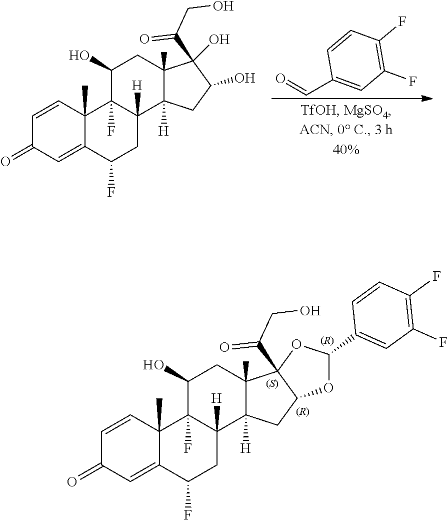

To a solution of fluocinolone (100.0 mg, 0.24 mmol, 1.0 equiv) and 3,4-difluorobenzaldehyde (51.7 mg, 0.36 mmol, 1.5 equiv) in acetonitrile (1 mL) with an inert atmosphere of nitrogen, was added magnesium sulfate (20.6 mg, 0.85 mmol, 3.5 equiv) in portions at room temperature under. To the above mixture was added trifluoromethanesulfonic acid (109.2 mg, 0.73 mmol, 3.0 equiv) in portions over 1 min at 0° C. The resulting mixture was stirred for additional 3 h at 0° C. The resulting solution was purified by Flash-Prep-HPLC with the following conditions (IntelFlash-1): Column, C₁₈ silica gel; mobile phase, water (with 5 mmol/L trifluoroacetic acid) and acetonitrile (5.0% acetonitrile up to 100.0% in 20 min); Detector, UV 254 nm. 26 mg (40%) of (2S,6aS,6bR,7S,8aS,8bS,10R,11aR,12aS,12bS)-10-(3,4-difluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as white solid: MS m/z [M+H]⁺ (ESI): 537.25; ¹H NMR (400 MHz, dimethyl sulfoxide-d₆+deuterium oxide) δ: 0.85 (s, 3H), 1.50-1.54 (m, 4H), 1.69-1.75 (m, 3H), 1.99-2.04 (m, 1H), 2.13-2.18 (m, 1H), 2.34-2.35 (m, 1H), 2.63-2.70 (m, 1H), 4.19-4.25 (m, 2H), 4.56 (d, J=19.6 Hz, 1H), 4.98 (s, 1H), 5.53-5.71 (m, 2H), 6.16 (s, 1H), 6.31-6.33 (m, 1H), 7.27-7.33 (m, 2H), 7.44-7.53 (m, 2H).

### Example 13. Preparation of (2S,6aS,6bR,7S,8aS,8bS,10R,11aR,12aS,12bS)-2,6b-difluoro-7-hydroxy-8b-(2-hydroxyacetyl)-10-isopropyl-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

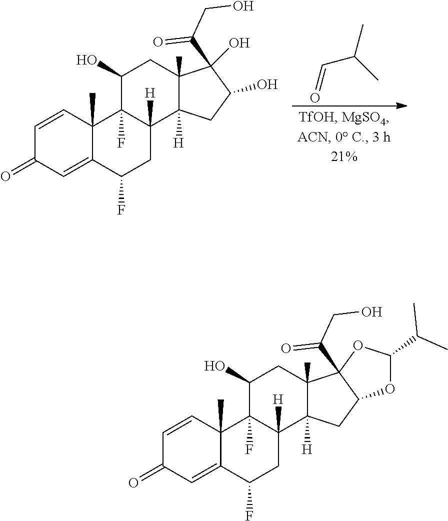

To a solution of fluocinolone (100 mg, 0.24 mmol, 1.0 equiv) in acetonitrile (1.0 mL) with an inert atmosphere of nitrogen, was added isobutyraldehyde (26.2 mg, 0.40 mmol, 1.5 equiv), magnesium sulfate (102.1 mg, 0.80 mmol, 3.5 equiv) for 3 min at 0° C. followed by the addition of trifluoromethanesulfonic acid (109.2 mg, 0.70 mmol, 3.0 equiv) dropwise at 0° C. The resulting solution was stirred for 3 h at 0° C., adjusted pH value to 7 with saturated sodium bicarbonate solution (1.0 mL). The mixture was diluted with 5 mL of water and extracted with 3×50 mL of dichloromethane. The organic layers were combined, washed with 3×50 mL of saturated sodium chloride, dried over anhydrous sodium sulfate, filtered, and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 23.4 mg (21%) of (2S,6aS,6bR,7S,8aS,8bS,10R,11aR,12aS,12bS)-2,6b-difluoro-7-hydroxy-8b-(2-hydroxyacetyl)-10-isopropyl-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 467.15; ¹H NMR (400 MHz, Methanol-d₄) δ: 0.93-0.95 (m, 9H), 1.52-1.59 (m, 4H), 1.65-1.69 (m, 3H), 1.82-1.86 (m, 1H), 2.21-2.34 (m, 3H), 2.59-2.67 (m, 1H), 4.26-4.30 (m, 2H), 4.39 (s, 1H), 4.51 (d, J=19.6 Hz, 1H), 4.88-4.89 (m, 1H), 5.47-5.59 (m, 1H), 6.29-6.35 (m, 2H), 7.30-7.33 (m, 1H).

### Example 14. Preparation of (2S,6aS,6bR,7S,8aS,8bS,10R,11aR,12aS,12bS)-10-cyclobutyl-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

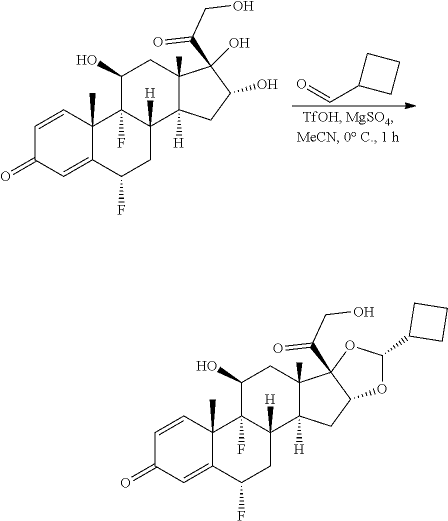

To a solution of fluocinolone (2.00 g, 4.85 mmol, 1.0 eq) in MeCN (20 mL) was added MgSO₄ (2.33 g, 19.4 mmol, 4.0 eq) at 0° C. After addition, the mixture was stirred at this temperature for 10 min, and then cyclobutanecarbaldehyde (612 mg, 7.27 mmol, 1.5 eq), TfOH (2.18 g, 14.6 mmol, 1.28 mL, 3.0 eq) was added dropwise at 0° C. The resulting mixture was stirred at 0° C. for 1 hour. The resulting solution was quenched until pH to ˜7 with saturated sodium bicarbonate solution. The mixture was diluted with 30 mL of water and extracted with 3×30 mL of dichloromethane and the organic layers were combined, washed with 40 mL of saturated sodium chloride solution, dried over anhydrous sodium sulfate, filtered, and concentrated under reduced pressure. The residue was purified by prep-HPLC (column: Phenomenex luna C₁₈ (250×70 mm, 15 um); mobile phase: [water (TFA)-ACN]; B %: 38% 58%, 23 min). (2S,6aS,6bR,7S,8aS,8bS,10R,11aR,12aS,12bS)-10-cyclobutyl-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (1.75 g, 3.60 mmol, 74.3% yield, 98.5% purity) was obtained as a white solid: MS m/z [M+H]+ (ESI): 479.3; ¹H NMR (400 MHz, Methanol-d₄) δ: 7.26 (dd, J=1.2, 10.0 Hz, 1H), 6.29 (dd, J=1.6, 10.0 Hz, 1H), 6.10 (s, 1H), 5.73-5.53 (m, 1H), 5.50 (d, J=2.8 Hz, 1H), 5.30-5.03 (m, 1H), 4.81 (t, J=2.4 Hz, 1H), 4.53 (d, J=3.6 Hz, 1H), 4.42 (d, J=19.6 Hz, 1H), 4.23-4.13 (m, 2H), 2.70-2.53 (m, 2H), 2.31-2.21 (m, 1H), 2.14-2.01 (m, 2H), 1.91-1.84 (m, 4H), 1.83-1.75 (m, 2H), 1.72-1.56 (m, 3H), 1.52 (s, 3H), 1.45-1.35 (m, 1H), 0.84-0.79 (s, 3H).

### Example 15. Preparation of (2S,6aS,6bR,7S,8aS,8bS,10R,11aR,12aS,12bS)-10-(bicyclo[1.1.1]pentan-1-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

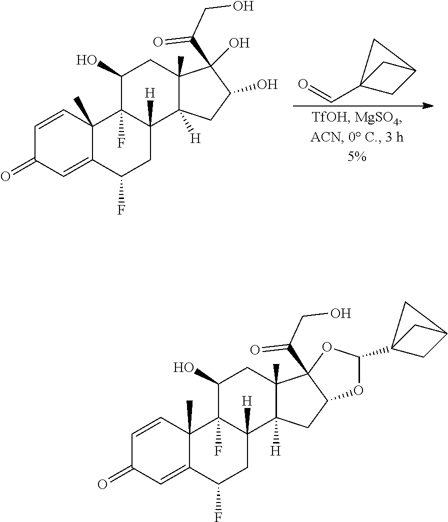

To a stirred solution of fluocinolone (100.0 mg, 0.20 mmol, 1.0 equiv), bicyclo[1.1.1]pentane-1-carbaldehyde (93.2 mg, 1.00 mmol, 4.0 equiv) and magnesium sulfate (102.0 mg, 0.7 mmol, 3.5 equiv) in acetonitrile (2.0 mL) were added trifluoromethanesulfonic acid (108.0 mg, 0.60 mmol, 3.0 equiv) at 0° C. under. The resulting mixture was stirred for 3 h at 0° C. The reaction was monitored by LCMS. The mixture was neutralized to pH 7 with saturated sodium bicarbonate (aq.). The aqueous layer was extracted with methylene chloride (3×30 mL). The solvent was removed by distillation under vacuum. The crude product was purified by reverse phase flash with the following conditions (column, C₁₈ silica gel; mobile phase, acetonitrile in water (10 mmol/L ammonium bicarbonate), 10% to 100% gradient in 20 min; detector, UV 254 nm) to afford (2S,6aS,6bR,7S,8aS,8bS,10R,11aR,12aS,12bS)-10-(bicyclo[1.1.1]pentan-1-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (6.0 mg, 5%) as a white solid: MS m/z [M+H]⁺ (ESI): 491.30; ¹H NMR (300 MHz, DMSO-d₆) δ: 0.75-0.85 (m, 3H), 1.45 (s, 3H), 1.71-1.78 (m, 10H), 1.94-1.99 (m, 2H), 2.20-2.32 (m, 1H), 2.45-2.46 (m, 2H), 4.10-4.19 (m, 2H), 4.35-4.37 (m, 1H), 4.51-4.59 (m, 1H), 5.03-5.14 (m, 2H), 5.49-5.53 (m, 2H), 6.11 (s, 1H), 6.28-6.32 (m, 1H), 7.24-7.28 (m, 1H).

### Example 16. Preparation of (2S,6aS,6bR,7S,8aS,8bS,10R,11aR,12aS,12bS)-2,6b-difluoro-10-(3-fluorophenyl)-8b-glycyl-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

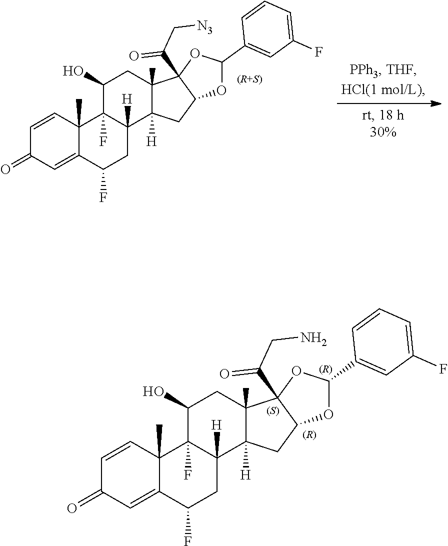

To a solution of (2S,6aS,6bR,7S,8aS,8bS,11aR,12aS,12bS)-8b-(2-azidoacetyl)-2,6b-difluoro-10-(3-fluorophenyl)-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (20.0 mg, 0.04 mmol, 1.0 equiv) and triphenylphosphine (14.5 mg, 0.06 mmol, 1.5 equiv) in tetrahydrofuran (1 mL) was added hydrochloric acid (2.7 mg, 0.08 mmol, 2.0 equiv) dropwise at room temperature under nitrogen atmosphere. The resulting mixture was stirred for 18 h at room temperature under nitrogen atmosphere. The crude product was purified by Flash-Prep-HPLC with the following conditions (IntelFlash-1): Column, C₁₈ silica gel; mobile phase, water (with 0.5% trifluoroacetic acid) and acetonitrile (10.0% acetonitrile up to 100.0% in 30 min); Detector, UV 254 nm. 7 mg (30%) of (2S,6aS,6bR,7S,8aS,8bS,10R,11aR,12aS,12bS)-2,6b-difluoro-10-(3-fluorophenyl)-8b-glycyl-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as white solid: MS m/z [M+H]⁺ (ESI): 518.55.

### Preparation of (2S,6aS,6bR,7S,8aS,8bS,11 aR,12aS,12bS)-2,6b-difluoro-1 O-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

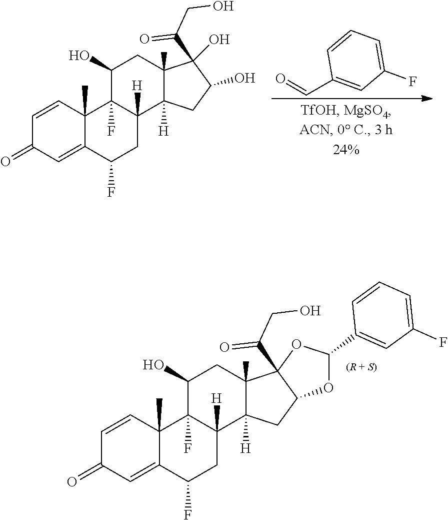

To a solution of fluocinolone (500.0 mg, 1.00 mmol, 1.0 equiv) and benzaldehyde, 3-fluoro- (225.0 mg, 1.50 mmol, 1.5 equiv) in acetonitrile (5 mL) were added trifluoro(sulfonyl)methane (544.0 mg, 3.0 mmol, 3.0 equiv) and magnesium sulfate (508.0 mg, 3.50 mmol, 3.5 equiv) dropwise at 0° C. under nitrogen atmosphere. The mixture was neutralized to pH 7 with saturated sodium bicarbonate (aq.). The aqueous layer was extracted with methylene chloride (3×30 mL). The solvent was removed by distillation under vacuum. The crude product was purified by Flash-Prep-HPLC with the following conditions (IntelFlash-1): Column, C₁₈ silica gel; mobile phase, water and acetonitrile (10.0% acetonitrile up to 100.0% in 30 min); Detector, UV 254 nm. 200.0 mg (24%) of (2S,6aS,6bR,7S,8aS,8bS,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as yellow oil: MS m/z [M+H]⁺ (ESI): 519.53.

### Preparation of 2-((2S,6aS,6bR,7S,8aS,8bS,11 aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl methanesulfonate

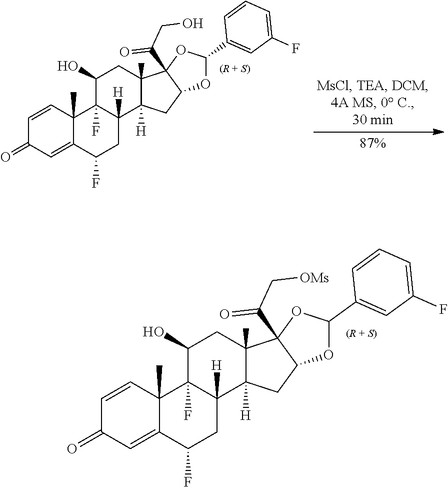

To a solution of (2S,6aS,6bR,7S,8aS,8bS,11aR,12aS,12bS)-2,6b-difluoro-10-(3-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (200.0 mg, 0.39 mmol, 1.0 equiv) and triethylamine (117.1 mg, 1.16 mmol, 3.0 equiv) in dichloromethane (5 mL) was added methanesulfonyl chloride (53.0 mg, 0.46 mmol, 1.2 equiv) dropwise at 0° C. under nitrogen atmosphere. The resulting mixture was stirred for 30 min at 0° C. under nitrogen atmosphere. The solvent was removed by distillation under vacuum. The crude product was purified by Flash-Prep-HPLC with the following conditions (IntelFlash-1): Column, C₁₈ silica gel; mobile phase, water and acetonitrile (10.0% acetonitrile up to 100.0% in 30 min); Detector, UV 254 nm. 200 mg (87%) of 2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl methanesulfonate was obtained as yellow solid: MS m/z [M+H]⁺ (ESI): 597.61.

### Preparation of (2S,6aS,6bR,7S,8aS,8bS,11 aR,12aS,12bS)-8b-(2-azidoacetyl)-2,6b-difluoro-10-(3 fluorophenyl)-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

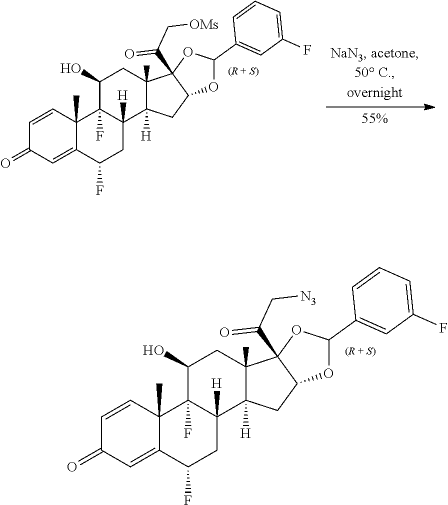

To a solution of 2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a,11a, 12,12a,12b-dodecahydro-8bH-naphtho[2′,1′:4,5]indeno[1,2-d][1,3]dioxol-8b-yl)-2-oxoethyl methanesulfonate (60.0 mg, 0.10 mmol, 1.0 equiv) and sodium azide (9.8 mg, 0.16 mmol, 1.5 equiv) in acetone was stirred for overnight at 50° C. under nitrogen atmosphere. The crude product was purified by Flash-Prep-HPLC with the following conditions (IntelFlash-1): Column, C₁₈ s silica gel; mobile phase, water and acetonitrile (10.0% acetonitrile up to 100.0% in 30 min); Detector, UV 254 nm. 30 mg (55%) of (2S,6aS,6bR,7S,8aS,8bS,11aR,12aS,12bS)-8b-(2-azidoacetyl)-2,6b-difluoro-10-(3-fluorophenyl)-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as yellow solid: MS m/z [M+H]⁺ (ESI): 544.54.

### Example 17. Preparation of (2S,6aS,6bR,7S,8aS,8bS,10R,11aR,12aS,12bS)-2,6b-difluoro-10-(3-fluoro-4-hydroxyphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

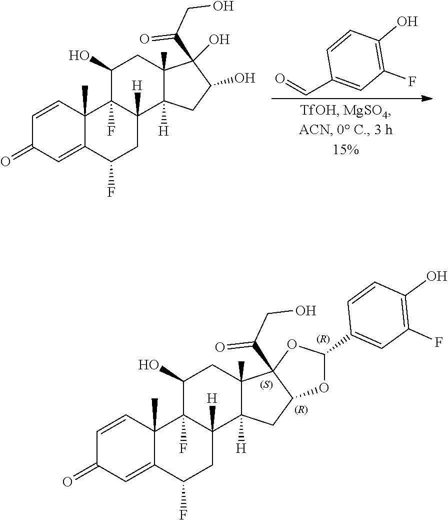

To a stirred solution of fluocinolone (100.0 mg, 0.24 mmol, 1.0 equiv) and 3-fluoro-4-hydroxybenzaldehyde (50.9 mg, 0.36 mmol, 1.5 equiv) in acetonitrile (1 mL) were added magnesium sulfate (102.1 mg, 0.84 mmol, 3.5 equiv) and trifluoromethanesulfonic acid (109.1 mg, 0.72 mmol, 3.0 equiv) dropwise at 0° C. under nitrogen atmosphere. The mixture was neutralized to pH 7 with saturated sodium bicarbonate (aq.). The aqueous layer was extracted with methylene chloride (3×30 mL). The solvent was removed by distillation under vacuum. The residue was purified by reverse flash chromatography with the following conditions: column, C₁₈ silica gel; mobile phase, acetonitrile in water (0.1% ammonium hydroxide+10 mmol/L ammonium bicarbonate), 10% to 100% gradient in 20 min; detector, UV 254 nm. This resulted in (2S,6aS,6bR,7S,8aS,8bS,10R,11aR,12aS,12bS)-2,6b-difluoro-10-(3-fluoro-4-hydroxyphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (22 mg, 15%) as a white solid: MS m/z [M−H]⁻ (ESI): 533.00; ¹H NMR (300 MHz, Methanol-d₄) δ: 1.00 (s, 3H), 1.61 (s, 3H), 1.66-1.85 (m, 4H), 2.26-2.46 (m, 3H), 2.67-2.78 (m, 1H), 4.31-4.37 (m, 2H), 4.64 (d, J=19.5 Hz, 1H), 5.05-5.06 (m, 1H), 5.43 (s, 1H), 5.47-5.67 (m, 1H), 6.35-6.39 (m, 2H), 6.89-6.95 (m, 1H), 7.09-7.17 (m, 2H), 7.33-7.37 (m, 1H).

### Example 18. Preparation of (2S,6aS,6bR,7S,8aS,8bS,10R,11aR,12aS,12bS)-2,6b-difluoro-10-(3-fluoro-4-phenoxyphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

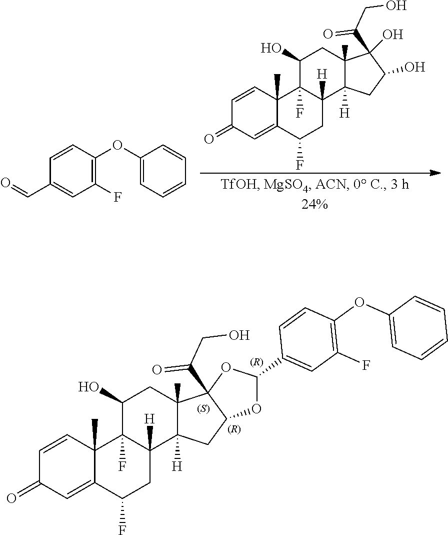

To a solution of 3-fluoro-4-phenoxybenzaldehyde (30.0 mg, 0.14 mmol, 1.0 equiv) and fluocinolone (85.8 mg, 0.21 mmol, 1.5 equiv) in acetonitrile (10 mL/g) was added magnesium sulfate (66.8 mg, 0.56 mmol, 4.0 equiv), followed by trifluoro(sulfonyl)methane (62.5 mg, 0.42 mmol, 3.0 equiv) was added dropwise at 0° C. under nitrogen atmosphere. The resulting mixture was stirred for 3 h at 0° C. The aqueous layer was extracted with methylene chloride (3×20 mL). The resulting mixture was concentrated under reduced pressure. The residue was purified by reverse flash chromatography with the following conditions: column, C₁₈ silica gel; mobile phase, acetonitrile in water (0.1% trifluoroacetic acid), 5% to 100% gradient in 30 min; detector, UV 254 nm. This resulted in (2S,6aS,6bR,7S,8aS,8bS,10R,11aR,12aS,12bS)-2,6b-difluoro-10-(3-fluoro-4-phenoxyphenyl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (21 mg, 24%) as a white solid: MS m/z [M+H]⁺ (ESI): 611.25; ¹H NMR (400 MHz, Methanol-d₄) δ: 1.00 (s, 3H), 1.55-1.85 (m, 7H), 2.24-2.38 (m, 3H), 2.63-2.76 (m, 1H), 4.31-4.37 (m, 2H), 4.65 (d, J=19.2 Hz, 1H), 5.08-5.09 (m, 1H), 5.48-5.60 (m, 2H), 6.31-6.34 (m, 2H), 6.93 (s, 2H), 7.01-7.08 (m, 2H), 7.24-7.42 (m, 5H).

### Preparation of 3 fluoro-4-phenoxybenzaldehyde

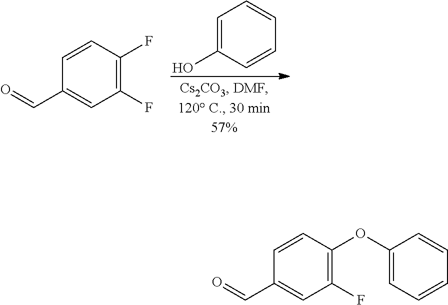 [SMILES: O=Cc1ccc(F)c(F)c1 ; Oc1ccccc1 ; O=Cc1ccc(Oc2ccccc2)c(F)c1]

To a solution of 3,4-difluorobenzaldehyde (100.0 mg, 0.70 mmol, 1.0 equiv) and phenol (99.3 mg, 1.06 mmol, 1.5 equiv) in N,N-dimethylformamide (10 mL/g) was added cesium carbonate (687.8 mg, 2.11 mmol, 3.0 equiv) dropwise at 120° C. under nitrogen atmosphere. The resulting mixture was stirred for 0.5 h at 120° C. The aqueous layer was extracted with methylene chloride (3×30 mL). The resulting mixture was concentrated under reduced pressure. The residue was purified by reverse flash chromatography with the following conditions: column, C₁₈ silica gel; mobile phase, acetonitrile in water (0.1% trifluoroacetic acid), 10% to 100% gradient in 20 min; detector, UV 254 nm. This resulted in 3-fluoro-4-phenoxybenzaldehyde (87 mg, 57%) as a white solid: MS m/z [M+H]⁺ (ESI): 217.06.

### Example 19. Preparation of (2S,6aS,6bR,7S,8aS,8bS,10R,11aR,12aS,12bS)-10-(4-(3-amino-2,2-difluoropropoxy)-3-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

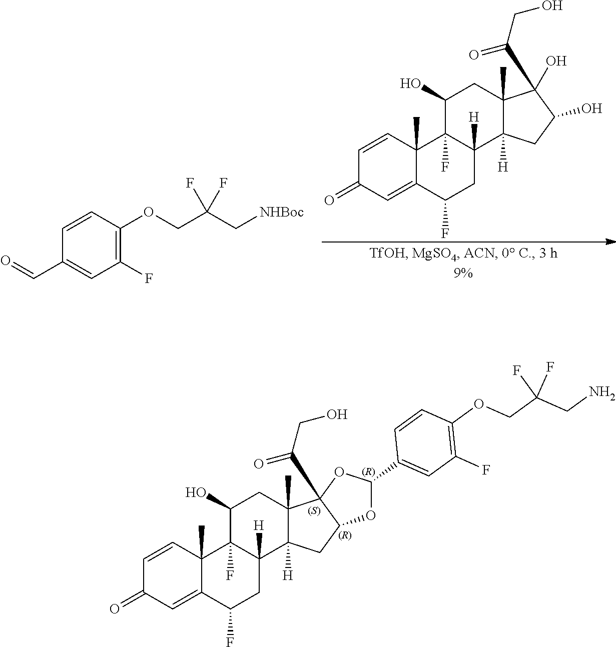

To a solution of tert-butyl (2,2-difluoro-3-(2-fluoro-4-formylphenoxy)propyl)carbamate (40.0 mg, 0.12 mmol, 1.0 equiv) and fluocinolone (49.5 mg, 0.12 mmol, 1.0 equiv), magnesium sulfate (50.6 mg, 0.42 mmol, 3.5 equiv) in acetonitrile (1 mL) was added trifluoromethanesulfonic acid (63.0 mg, 0.42 mmol, 3.5 equiv) dropwise at 0° C. under nitrogen atmosphere. The resulting mixture was stirred for 3 h at 0° C. under nitrogen atmosphere. The mixture was neutralized to pH 7 with saturated sodium bicarbonate. The residue was purified by reversed-phase flash chromatography with the following conditions: column, C₁₈ silica gel; mobile phase, acetonitrile in water (0.1% formic acid), 10% to 100% gradient in 30 min; detector, UV 254 nm. This resulted in (2S,6aS,6bR,7S,8aS,8bS,10R,11aR,12aS,12bS)-10-(4-(3-amino-2,2-difluoropropoxy)-3-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (7.8 mg, 9%) as a yellow solid: MS m/z [M+H]⁺ (ESI): 628.30; ¹H NMR (300 MHz, Methanol-d₄) δ: 0.98 (s, 3H), 1.58-1.81 (m, 7H), 2.22-2.27 (m, 1H), 2.30-2.41 (m, 2H), 2.65-2.70 (m, 1H), 3.16-3.26 (m, 2H), 4.29-4.41 (m, 4H), 4.63 (d, J=19.5 Hz, 1H), 5.05-5.06 (m, 1H), 5.47-5.60 (m, 2H), 6.32-6.36 (m, 2H), 7.14-7.33 (m, 4H).

### Preparation of tert-butyl (2,2-difluoro-3-(2 fluoro-4 formylphenoxy)propyl)carbamate d][1,3]dioxol-4-one

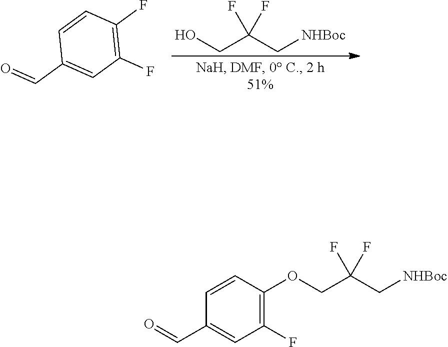 [SMILES: O=Cc1ccc(F)c(F)c1 ; CC(C)(C)OC(=O)NCC(F)(F)CO ; CC(C)(C)OC(=O)NCC(F)(F)COc1ccc(C=O)cc1F]

To a solution of 3,4-difluorobenzaldehyde (100.0 mg, 0.70 mmol, 1.0 equiv) and sodium hydride (25.3 mg, 1.06 mmol, 1.5 equiv) in N,N-dimethylformamide (1 mL) was added tert-butyl N-(2,2-difluoro-3-hydroxypropyl)carbamate (178.4 mg, 0.85 mmol, 1.2 equiv) dropwise at 0° C. under nitrogen atmosphere. The resulting mixture was stirred for 2 h at 0° C. under nitrogen atmosphere. The residue was purified by reversed-phase flash chromatography with the following conditions: column, C₁₈ silica gel; mobile phase, acetonitrile in water, 10% to 100% gradient in 30 min; detector, UV 254 nm. This resulted in tert-butyl (2,2-difluoro-3-(2-fluoro-4-formylphenoxy)propyl)carbamate (120 mg, 51%) as a white solid: MS m/z [M+H]⁺ (ESI): 334.31.

### Example 20. Preparation of (2S,6aS,6bR,7S,8aS,8bS,10R,11aR,12aS,12bS)-2,6b-difluoro-10-(3-fluoro-4-(methoxymethyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

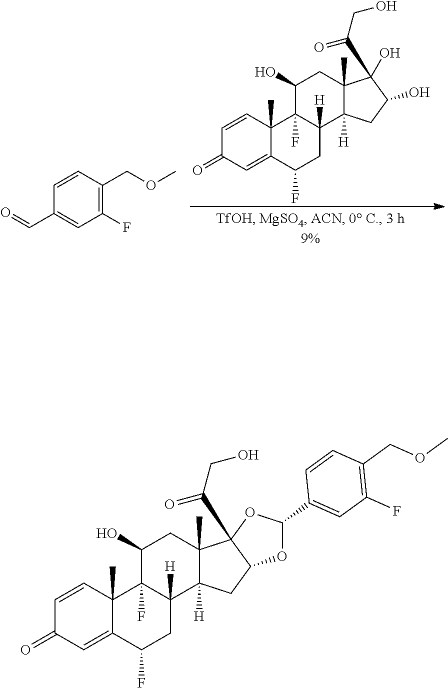

To a stirred solution of 3-fluoro-4-(methoxymethyl)benzaldehyde (100.0 mg, 0.6 mmol, 2.5 equiv) and fluocinolone (98.0 mg, 0.2 mmol, 1.0 equiv) in acetonitrile (1 mL) were added magnesium sulfate (99.9 mg, 0.8 mmol, 3.5 equiv) and trifluoromethanesulfonic acid (103.5 mg, 0.7 mmol, 3.0 equiv) dropwise in portions at 0° C. under nitrogen atmosphere. The resulting mixture was stirred for 3 h at room temperature under nitrogen atmosphere. The mixture was neutralized to pH 7 with saturated sodium bicarbonate (aq.). The resulting mixture was extracted with dichloromethane (3×30 mL). The combined organic layers were washed with water (3×30 mL), dried over anhydrous magnesium sulfate. After filtration, the filtrate was concentrated under reduced pressure. The crude product was purified by Prep-HPLC with the following conditions (column, C₁₈ silica gel; mobile phase, acetonitrile in water (0.1% ammonium hydroxide+I0 mmol/L ammonium bicarbonate) to afford (2S,6aS,6bR,7S,8aS,8bS,10R,11aR,12aS,12bS)-2,6b-difluoro-10-(3-fluoro-4-(methoxymethyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (13 mg, 9%) as a white solid: MS m/z [M−H]⁻ (ESI): 561.25; ¹H NMR (300 MHz, Methanol-d₄) δ: 1.00 (s, 3H), 1.60 (s, 4H), 1.72-1.84 (m, 3H), 2.35-2.39 (m, 3H), 2.63-2.80 (m, 1H), 3.39 (s, 3H), 4.33-4.39 (m, 2H), 4.53 (s, 2H), 4.67 (d, J=19.5 Hz, 1H), 5.10-5.12 (m, 1H), 5.46-5.69 (m, 2H), 6.34-6.39 (m, 2H), 7.18-7.22 (m, 1H), 7.29-7.36 (m, 2H), 7.45-7.49 (m, 1H).

### Preparation of 3 fluoro-4-(methoxymethyl)benzonitrile

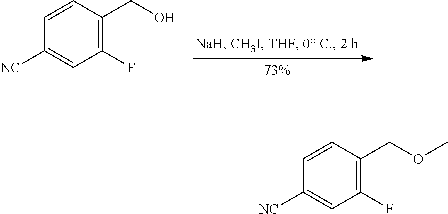

A solution of 3-fluoro-4-(hydroxymethyl)benzonitrile (1.0 g, 6.6 mol, 1.0 equiv) in tetrahydrofuran (10 mL) was treated with sodium hydride (190.5 mg, 7.92 mol, 1.2 equiv) for 1 h at 0° C. under nitrogen atmosphere followed by the addition of iodomethane (1.1 g, 7.92 mol, 1.2 equiv) dropwise at 0° C. The reaction was quenched with water at 0° C. The crude product was purified by reverse phase flash with the following conditions (column, C₁₈ silica gel; mobile phase, acetonitrile in water (0.1% ammonium hydroxide+10 mmol/L ammonium bicarbonate), 10% to 100% gradient in 20 min; detector, UV 254 nm) to afford 3-fluoro-4-(methoxymethyl)benzonitrile (800 mg, 73%) as a yellow oil: MS m/z [M+H]⁺ (ESI): 166.17.

### Preparation of 3 fluoro-4-(methoxymethyl)benzaldehyde

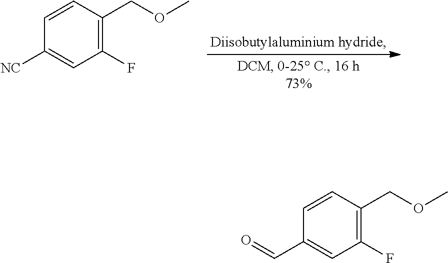

A solution of 3-fluoro-4-(methoxymethyl)benzonitrile (400.0 mg, 2.4 mmol, 1.0 equiv) and aluminium hydride (1 mol/L n-hexane) (516.6 mg, 3.6 mmol, 1.5 equiv) in acetonitrile (4 mL) was stirred for 5 min at 0° C. under nitrogen atmosphere. The resulting mixture was stirred for 16 h at room temperature under nitrogen atmosphere. The crude product was purified by reverse phase flash with the following conditions (column, C₁₈ silica gel; mobile phase, acetonitrile in water (0.1% ammonium hydroxide+10 mmol/L ammonium bicarbonate) to afford 3-fluoro-4-(methoxymethyl)benzaldehyde (300 mg, 73%) as a white solid: MS m/z [M+H]⁺ (ESI): 169.17.

### Example 21. Preparation of (2S,6aS,6bR,7S,8aS,8bS,10R,11aR,12aS,12bS)-2,6b-difluoro-10-(3-fluoro-4-(phenoxymethyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

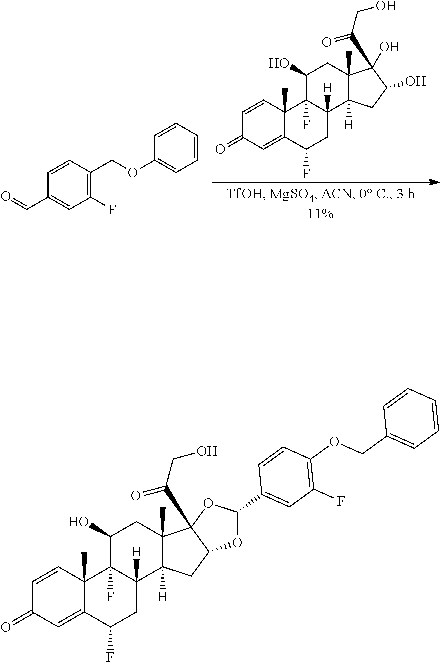

To a solution of 3-fluoro-4-(phenoxymethyl)benzaldehyde (100.0 mg, 0.4 mmol, 1.0 equiv) and fluocinolone (268.7 mg, 0.7 mmol, 1.5 equiv), magnesium sulfate (209.1 mg, 1.7 mmol, 4.0 equiv) in acetonitrile (1 mL) was added trifluoro(sulfonyl)methane (195.5 mg, 1.3 mmol, 3.0 equiv) dropwise 0° C. under nitrogen atmosphere. The residue was neutralized to pH 7 with saturated sodium bicarbonate. The aqueous layer was extracted with methylene chloride (3×30 mL). The residue was purified by reverse flash chromatography with the following conditions: column, C₁₈ silica gel; mobile phase, acetonitrile in water (0.1% ammonium hydroxide+10 mmol/L ammonium bicarbonate), 10% to 100% gradient in 30 min; detector, UV 254 nm. This resulted in (2S,6aS,6bR,7S,8aS,8bS,10R,11aR,12aS,12bS)-2,6b-difluoro-10-(3-fluoro-4-(phenoxymethyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (30 mg, 11%) as a white solid: MS m/z [M+H]⁺ (ESI): 625.25; ¹H NMR (400 MHz, Methanol-d₄) δ: 0.99 (s, 3H), 1.58-1.82 (m, 7H), 2.23-2.39 (m, 3H), 2.60-2.70 (m, 1H), 4.30-4.37 (m, 2H), 4.65 (d, J=19.6 Hz, 1H), 5.08-5.11 (m, 3H), 5.53-5.60 (m, 2H), 6.32-6.35 (m, 2H), 6.91-6.98 (m, 3H), 7.21-7.33 (m, 5H), 7.52-7.54 (m, 1H).

### Preparation of 3 fluoro-4-(phenoxymethyl)benzonitrile

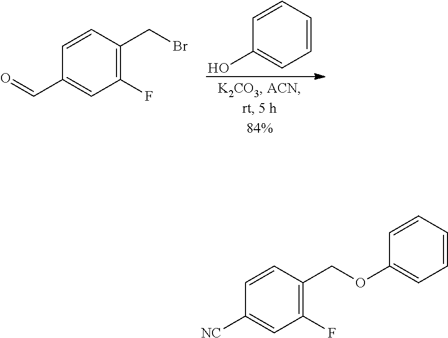 [SMILES: O=Cc1ccc(CBr)c(F)c1 ; Oc1ccccc1 ; N#Cc1ccc(COc2ccccc2)c(F)c1]

To a solution of 4-(bromomethyl)-3-fluorobenzonitrile (1.0 g, 4.7 mmol, 1.0 equiv) and potassium carbonate (1.3 g, 9.3 mmol, 2.0 equiv) in acetonitrile (10 mL) was added phenol (0.7 g, 7.0 mmol, 1.5 equiv) dropwise at room temperature under nitrogen atmosphere. The resulting mixture was stirred for 5 h at room temperature under nitrogen atmosphere. The aqueous layer was extracted with methylene chloride (3×100 mL). The resulting mixture was concentrated under reduced pressure. The residue was purified by reverse flash chromatography with the following conditions: column, C₁₈ silica gel; mobile phase, acetonitrile in water (0.1% Ammonium hydroxide), 10% to 100% gradient in 20 min; detector, UV 254 nm. This resulted in 3-fluoro-4-(phenoxymethyl)benzonitrile (0.9 g, 84%) as a yellow solid: MS m/z [M+H]⁺ (ESI): 228.07.

### Preparation of 3-fluoro-4-(phenoxymethyl)benzaldehyde

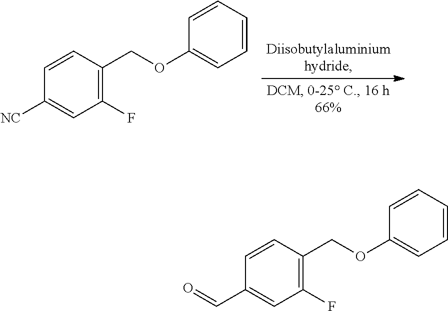 [SMILES: N#Cc1ccc(COc2ccccc2)c(F)c1 ; O=Cc1ccc(COc2ccccc2)c(F)c1]

To a solution of 3-fluoro-4-(phenoxymethyl)benzonitrile (600.0 mg, 2.6 mmol, 1.0 equiv) in methylene chloride (6 mL) was added bis(butan-2-yl)-1\hat{ }[2]-alumanyl (751.0 mg, 5.3 mmol, 2.0 equiv) dropwise at 0° C. under nitrogen atmosphere. The resulting mixture was stirred for 16 h at room temperature under nitrogen atmosphere. The residue was purified by Prep-TLC (petroleum ether/ethyl acetate 1:1) to afford PH-SVT-014-114-2. The resulting mixture was concentrated under reduced pressure. This resulted in 3-fluoro-4-(phenoxymethyl)benzaldehyde (400.0 mg, 66%) as a yellow solid: MS m/z [M+H]⁺ (ESI): 231.07.

### Example 22. Preparation of (2S,6aS,6bR,7S,8aS,8bS,10R,11aR,12aS,12bS)-2,6b-difluoro-10-(3-fluoro-4-methoxyphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

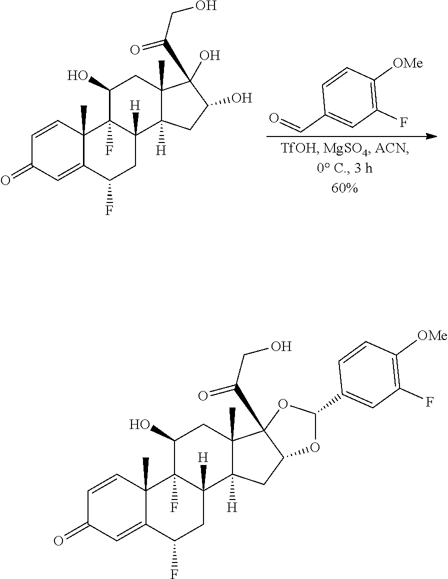

To a stirred solution of fluocinolone (30.0 mg, 0.07 mmol, 1.0 equiv) and 3-fluoro-4-methoxybenzaldehyde (16.8 mg, 0.1 mmol, 1.5 equiv) in acetonitrile were added trifluoro(sulfonyl)methane (32.8 mg, 0.22 mmol, 3.0 equiv) and magnesium sulfate (30.6 mg, 0.26 mmol, 3.5 equiv) dropwise at 0° C. under nitrogen atmosphere. The rude product was purified by reverse phase flash with the following conditions to afford (2S,6aS,6bR,7S,8aS,8bS,10R,11aR,12aS,12bS)-2,6b-difluoro-10-(3-fluoro-4-methoxyphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (24.7 mg, 60%) as a white solid: MS m/z [M+H]⁺ (ESI): 549.15; ¹H NMR (400 MHz, Methanol-d₄) δ: 0.98 (s, 3H), 1.58 (s, 3H), 1.59-1.81 (m, 4H), 2.23-2.25 (m, 1H), 2.27-2.39 (m, 2H), 2.60-2.75 (m, 1H), 3.85 (s, 3H), 4.29-4.35 (m, 2H), 4.63 (d, J=19.2 Hz, 1H), 5.04-5.05 (m, 1H), 5.44 (s, 1H), 5.55-5.65 (m, 1H), 6.33-6.36 (m, 2H), 7.07-7.17 (m, 2H), 7.21-7.24 (m, 1H), 7.31-7.33 (m, 1H).

### Example 23. Preparation of (2S,6aS,6bR,7S,8aS,8bS,10R,11aR,12aS,12bS)-8b-(2-(4-aminophenoxy)acetyl)-2,6b-difluoro-10-(3-fluorophenyl)-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

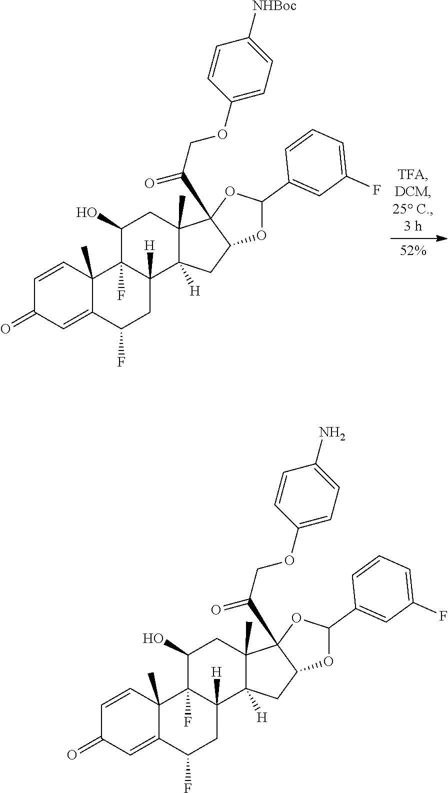

To a solution of tert-butyl (4-(2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)phenyl)carbamate (80.0 mg, 0.141 mmol, 1.0 equiv.) in dichloromethane (2.0 mL) with an inert atmosphere of nitrogen, was added trifluoroacetic acid (0.5 mL) at 25° C. The resulting solution was stirred for 3 h at 25° C. and concentrated under vacuum. The crude product was purified by Prep-HPLC with the following conditions (IntelFlash-1): Column: Xselect CSH F-Phenyl OBD column, 30×150 mm, 5 μm; Mobile Phase A: water, Mobile Phase B: acetonitrile; Flow rate: 60 mL/min; Gradient: 20% B to 50% B in 9 min, 62% B; Wave Length: 254 nm. The resulting mixture was lyophilized in a cool and dry place. 35.1 mg (52%) of (2S,6aS,6bR,7S,8aS,8bS,10R,11aR,12aS,12bS)-8b-(2-(4-aminophenoxy)acetyl)-2,6b-difluoro-10-(3-fluorophenyl)-7-hydroxy-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one was obtained as a yellow solid: MS m/z [M+H]⁺ (ESI): 610.15; ¹H NMR (400 MHz, Methanol-d₄) δ: 1.03 (s, 3H), 1.59-1.62 (m, 4H), 1.80-1.86 (m, 3H), 2.33-2.41 (m, 3H), 2.60-2.70 (m, 1H), 4.34-4.36 (m, 1H), 4.93-4.98 (m, 1H), 5.08-5.09 (m, 1H), 5.28 (d, J=18.0 Hz, 1H), 5.50-5.66 (m, 2H), 6.33-6.36 (m, 2H), 7.07-7.17 (m, 3H), 7.20-7.23 (m, 1H), 7.27-7.31 (m, 4H), 7.39-7.41 (m, 1H).

### Preparation of tert-butyl (4-(2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)phenyl)carbamate

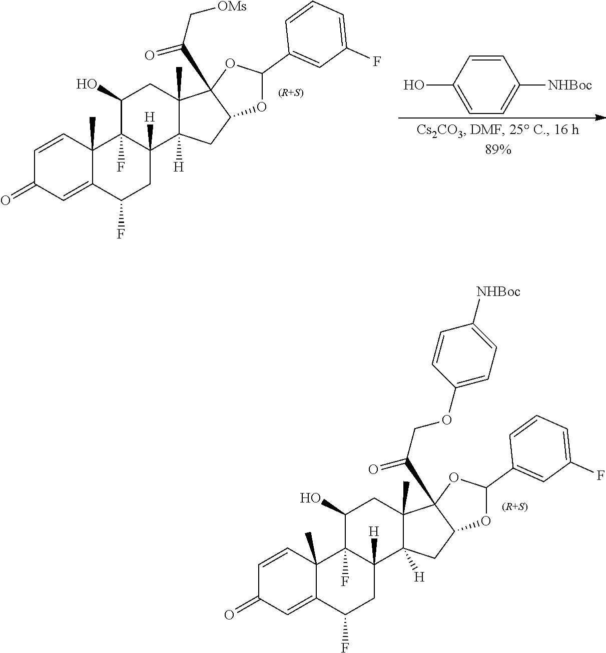

To a solution of 2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a,11a, 12,12a,12b-dodecahydro-8bH-naphtho[2′,1′:4,5]indeno[1,2-d][1,3]dioxol-8b-yl)-2-oxoethyl methanesulfonate (200.0 mg, 0.34 mmol, 3.0 equiv.) and tert-butyl N-(4-hydroxyphenyl) carbamate (23.4 mg, 0.11 mmol, 1.0 equiv.) in N, N-dimethylformamide (3.0 mL) was added cesium carbonate (109.22 mg, 0.34 mmol, 3.0 equiv.) at 25° C. under nitrogen atmosphere. The resulting solution was stirred for overnight at 25° C. The mixture was diluted with water (100 mL), extracted with dichloromethane (3×500 mL) and the organic layers were combined, washed with saturated sodium chloride solution (3×500 mL), dried over anhydrous sodium sulfate, filtered, and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 70 mg (89%) of tert-butyl (4-(2-((2S,6aS,6bR,7S,8aS,8bS,11aR,12aS,12bS)-2,6b-difluoro-10-(3-fluorophenyl)-7-hydroxy-6a,8a-dimethyl-4-oxo-1,2,4,6a,6b,7,8,8a, 11a, 12,12a,12b-dodecahydro-8bH-naphtho[2′, 1′: 4,5]indeno[1,2-d][1,3]dioxol-8b-yl)-2-oxoethoxy)phenyl)carbamate was obtained as a yellow oil: MS m/z [M+H]⁺ (ESI): 710.29.

### Example 24. Preparation of (2S,6aS,6bR,7S,8aS,8bS,10R,11aR,12aS,12bS)-2,6b-difluoro-10-(2-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

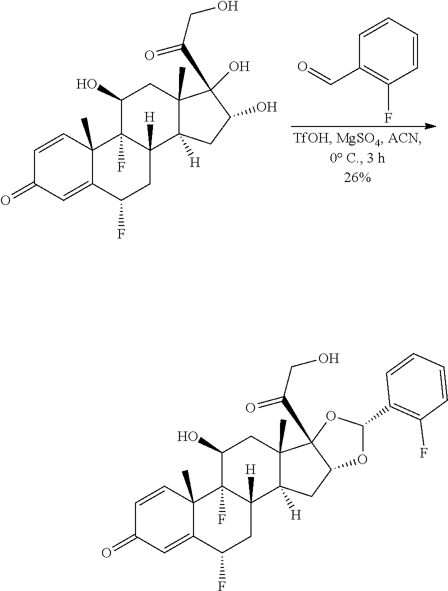

A solution of fluocinolone (100.0 mg, 0.24 mmol, 1.0 equiv) in acetonitrile (1 mL) was treated with 2-fluor-benzaldehyde (45.1 mg, 0.36 mmol, 1.5 equiv) for 5 min at 0° C. followed by the addition of magnesium sulfate (102.1 mg, 0.84 mmol, 3.5 equiv) dropwise at 0° C. The resulting mixture was stirred for 3 h at 0° C. under nitrogen atmosphere. The mixture was neutralized to pH 7 with saturated sodium bicarbonate (aq.). The resulting mixture was extracted with dichloromethane (3×30 mL). The combined organic layers were washed with water (3×10 mL), dried over anhydrous sodium sulfate. After filtration, the filtrate was concentrated under reduced pressure. The crude product was purified by Prep-HPLC with the following conditions (column, C₁₈ silica gel; mobile phase, acetonitrile in Water (0.1% ammonium hydroxide+10 mmol/L ammonium bicarbonate)) to afford (2S,6aS,6bR,7S,8aS,8bS,10R,11aR,12aS,12bS)-2,6b-difluoro-10-(2-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (33.9 mg, 26%) as a white solid: MS m/z [M+H]⁺ (ESI): 519.25; ¹H NMR (300 MHz, Methanol-d₄) δ: 0.99 (s, 3H), 1.58-1.73 (m, 5H), 1.77-1.83 (m, 2H), 2.22-2.27 (m, 1H), 2.34-2.39 (m, 2H), 2.65-2.70 (m, 1H), 4.28-4.31 (m, 2H), 4.65 (d, J=19.5 Hz, 1H), 5.08-5.10 (m, 1H), 5.50-5.65 (m, 1H), 5.77 (s, 1H), 6.31-6.35 (m, 2H), 7.08-7.19 (m, 1H), 7.21-7.29 (m, 1H), 7.33-7.39 (m, 1H), 7.40-7.43 (m, 1H), 7.55-7.60 (m, 1H).

### Example 25. Preparation of (2S,6aS,6bR,7S,8aS,8bS,10R,11aR,12aS,12bS)-10-(3,3-difluorocyclobutyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

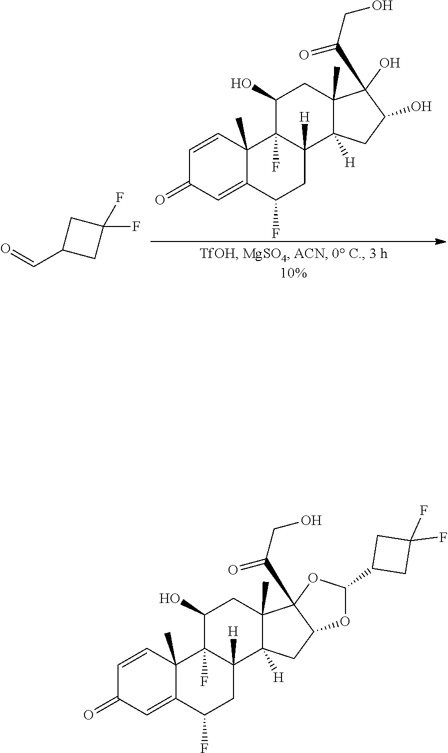

To a stirred solution of 3,3-difluorocyclobutane-1-carbaldehyde (50.0 mg, 0.4 mmol, 1.1 equiv) and fluocinolone (156.1 mg, 0.4 mmol, 1.0 equiv) and magnesium sulfate (136.7 mg, 1.2 mmol, 3.0 equiv) in acetonitrile (1 mL) was added trifluoromethanesulfonic acid (198.8 mg, 1.3 mmol, 3.5 equiv) dropwise at 0° C. under nitrogen atmosphere. The residue was basified to pH 7 with sodium bicarbonate. The crude product was purified by Prep-HPLC with the following conditions (acetonitrile/water) to afford (2S,6aS,6bR,7S,8aS,8bS,10R,11aR,12aS,12bS)-10-(3,3-difluorocyclobutyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (19.9 mg, 10.1%) as a white solid: MS m/z [M−H]⁻ (ESI): 513.20; ¹H NMR (300 MHz, Methanol-d₄) δ: 7.31 (dd, J=10.0, 1.5 Hz, 1H), 6.38-6.24 (m, 2H), 5.67-5.24 (m, 1H), 4.97-4.93 (m, 1H), 4.70 (d, J=3.3 Hz, 1H), 4.63-4.49 (m, 1H), 4.36-4.12 (m, 2H), 2.79-2.29 (m, 7H), 2.26-2.07 (m, 2H), 2.03 (s, 1H), 1.63 (d, J=40.7 Hz, 7H), 0.95 (d, J=7.6 Hz, 3H).

### Preparation of 3,3-difluorocyclobutane-1-carbaldehyde

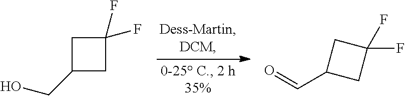 [SMILES: OCC1CC(F)(F)C1 ; O=CC1CC(F)(F)C1]

A solution of (3,3-difluorocyclobutyl)methanol (200.0 mg, 1.6 mmol, 1.0 equiv) in dichloromethane (2 mL) was treated with Dess-Martin (1042.0 mg, 2.5 mmol, 1.5 equiv) at 0° C. under nitrogen atmosphere. The temperature was increased to room temperature naturally. The residue was purified by reversed-phase flash chromatography with the following conditions: column, C₁₈ silica gel; mobile phase, acetonitrile in water, 10% to 100% gradient in 10 min; detector, UV 254 nm. The resulting mixture was concentrated under reduced pressure. This resulted in 3,3-difluorocyclobutane-1-carbaldehyde (70.0 mg, 35%) as a colorless oil.

### Example 26. Preparation of (2S,6aS,6bR,7S,8aS,8bS,10R,11aR,12aS,12bS)-2,6b-difluoro-10-(5-fluorothiophen-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

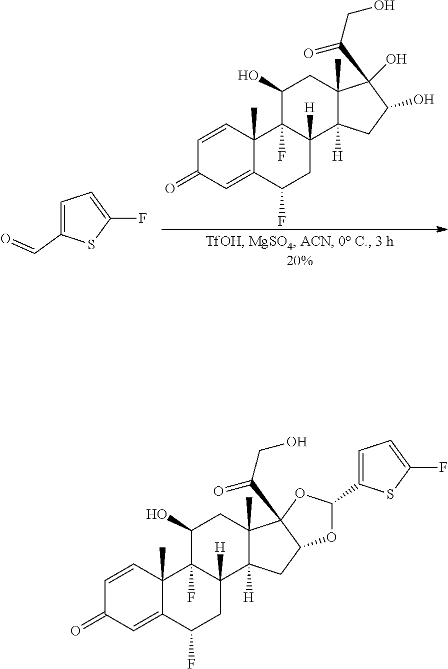

To a stirred solution of 5-fluorothiophene-2-carbaldehyde (50.0 mg, 0.38 mmol, 2.0 equiv), fluocinolone (79.2 mg, 0.19 mmol, 1.0 equiv) and magnesium sulfate (80.9 mg, 0.67 mmol, 3.5 equiv) in acetonitrile (2 mL) was added trifluoroacetic acid (65.7 mg, 0.58 mmol, 3.0 equiv) at 0° C. The resulting mixture was stirred for 3 h at 0° C. under. The crude product was purified by reverse phase flash with the following conditions (column, C₁₈ silica gel; mobile phase, acetonitrile in Water (10 mmol/L Ammonium bicarbonate), 10% to 100% gradient in 20 min; detector, UV 254 nm.) to afford (2S,6aS,6bR,7S,8aS,8bS,10R,11aR,12aS,12bS)-2,6b-difluoro-10-(5-fluorothiophen-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (20.0 mg, 20%) as a white solid: MS m/z [M+H]⁺ (ESI): 525.20; ¹H NMR (400 MHz, Methanol-d₄) δ: 0.99 (s, 3H), 1.58 (s, 3H), 1.62-1.78 (m, 4H), 2.21-2.24 (m, 1H), 2.31-2.39 (m, 2H), 2.64-2.74 (m, 1H), 4.29-4.33 (m, 2H), 4.62 (d, J=19.6 Hz, 1H), 5.03-5.04 (m, 1H), 5.49-5.64 (m, 1H), 5.72 (s, 1H), 6.33-6.43 (m, 3H), 6.87-6.89 (m, 1H), 7.31-7.35 (m, 1H).

### Preparation of 5-fluorothiophene-2-carbaldehyde

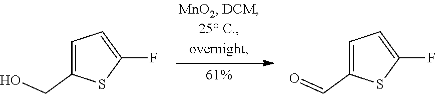 [SMILES: OCc1ccc(F)s1 ; O=Cc1ccc(F)s1]

To a stirred solution of (5-fluorothiophen-2-yl)methanol (100.0 mg, 0.76 mmol, 1.0 equiv) in dichloromethane (4.0 mL) was added manganese dioxide (657.8 mg, 7.6 mmol, 10 equiv) at room temperature. The resulting mixture was stirred for 12 h. The reaction was monitored by TLC. The resulting mixture was filtered, the filter cake was washed with dichloromethane. The filtrate was concentrated under reduced pressure to afford 5-fluorothiophene-2-carbaldehyde (60 mg, 61%) as a light yellow oil.

### Example 27. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-methyl-1H-indazol-5-yl)-1,2,6a,6b,7,8,8a,8b,11a,12,12a,12b-dodecahydro-4H-naphtho[2′,1′:4,5]indeno[1,2-d][1,3]dioxol-4-one

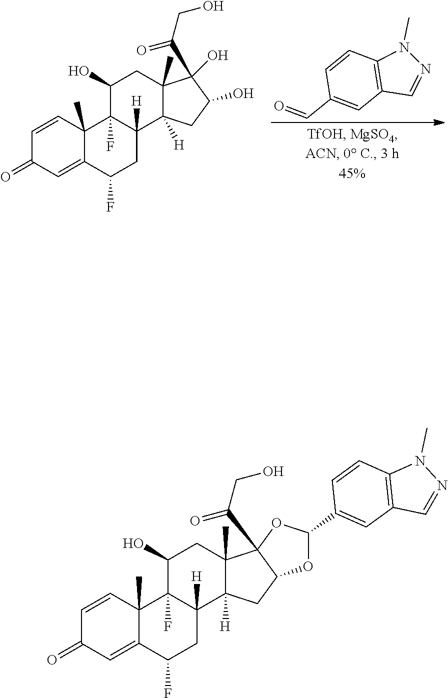

To a solution of fluocinolone (50.0 mg, 0.12 mmol, 1.0 equiv) and 1-methylindazole-5-carbaldehyde (29.1 mg, 0.18 mmol, 1.5 equiv) in acetonitrile (1 mL) were added trifluoro(sulfonyl)methane (72.8 mg, 0.48 mmol, 4.0 equiv) and magnesium sulfate (43.7 mg, 0.36 mmol, 3.0 equiv) dropwise at 0° C. under nitrogen atmosphere. The resulting mixture was stirred for 3 h at 0° C. under nitrogen atmosphere. The mixture was neutralized to pH 7 with saturated sodium bicarbonate. The aqueous layer was extracted with dichloromethane (3×50 mL). The crude product (50.0 mg) was purified by Prep-HPLC with the following conditions (Column: XBridge Prep OBD C₁₈ Column, 30×150 mm, 5 μm; Mobile Phase A: Water (10 mmol/L ammonium bicarbonate), Mobile Phase B: acetonitrile; Flow rate: 80 mL/min; Gradient: 5% B to 80% B in 30 min, to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-methyl-1H-indazol-5-yl)-1,2,6a,6b,7,8,8a,8b,11a,12,12a,12b-dodecahydro-4H-naphtho[2′,1′:4,5]indeno[1,2-d][1,3]dioxol-4-one (30.1 mg, 45%) as a white solid: MS m/z [M+H]⁺ (ESI): 555.30; ¹H NMR (300 MHz, DMSO-d₆) δ: 0.89 (s, 3H), 1.51-1.59 (m, 4H), 1.69-1.83 (m, 3H), 2.04-2.09 (m, 1H), 2.27-2.34 (m, 2H), 2.62-2.73 (m, 1H), 4.02 (s, 3H), 4.19-4.27 (m, 2H), 4.52-4.61 (m, 1H), 4.98-4.99 (m, 1H), 5.11-5.15 (m, 1H), 5.54-5.55 (m, 1H), 5.62 (s, 1H), 5.70-5.75 (m, 1H), 6.15 (s, 1H), 6.29-6.33 (m, 1H), 7.26-7.29 (m, 1H), 7.45-7.48 (m, 1H), 7.69-7.72 (m, 1H), 7.83 (s, 1H), 8.08 (s, 1H).

### Example 28. Preparation of (2S,6aS,6bR,7S,8aS,8bS,10R,11aR,12aS,12bS)-2,6b-difluoro-10-(3-fluorothiophene-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

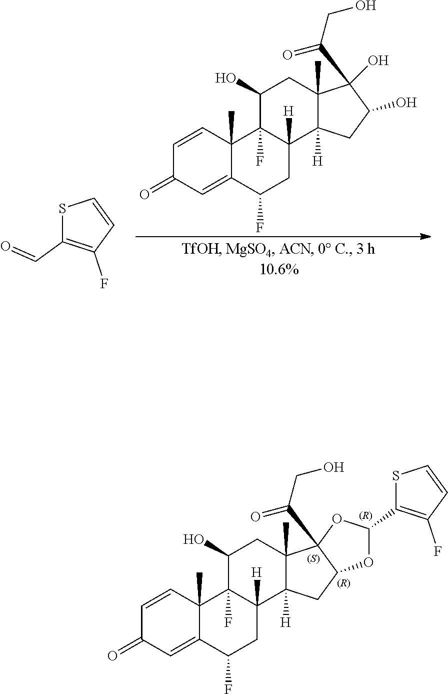

To a stirred solution of 3-fluorothiophene-2-carbaldehyde (40 mg, 0.307 mmol, 1 equiv) and fluocinolone (126.77 mg, 0.307 mmol, 1 equiv) in acetonitrile (1 mL) was added magnesium sulfate (129.48 mg, 1.075 mmol, 3.5 equiv), trifluoromethanesulfonic acid (138.38 mg, 0.921 mmol, 3 equiv) at 0° C. under nitrogen atmosphere. The crude product was purified by Prep-HPLC with the following conditions (acetonitrile/water with 0.05% ammonium bicarbonate) to afford (2S,6aS,6bR,7S,8aS,8bS,10R,11aR,12aS,12bS)-2,6b-difluoro-10-(3-fluorothiophen-2-yl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (17.2 mg, 10.6%) as a white solid: MS m/z [M+H]⁺ (ESI): 525.15; ¹H NMR (300 MHz, Methanol-d₄) δ: 7.46-7.35 (m, 1H), 7.32 (d, J=10.0 Hz, 1H), 6.82-6.75 (m, 1H), 6.33 (d, J=11.4 Hz, 2H), 5.93 (d, J=2.0 Hz, 1H), 5.69-5.38 (m, 1H), 5.05 (d, J=3.2 Hz, 1H), 4.62 (dd, J=19.4, 1.9 Hz, 1H), 4.46-4.23 (m, 2H), 2.83-2.55 (m, 1H), 2.49-2.30 (m, 2H), 2.24 (d, J=14.0 Hz, 1H), 1.87-1.48 (m, 7H), 0.97 (s, 3H).

### Example 29. Preparation of (2S,6aS,6bR,7S,8aS,8bS,10R,11aR,12aS,12bS)-2,6b-difluoro-10-(4-fluorothiophen-3-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

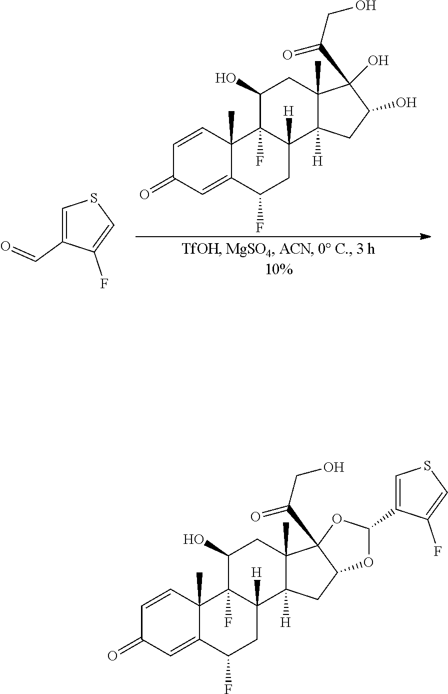

To a stirred solution of 4-fluorothiophene-3-carbaldehyde (60.0 mg, 0.5 mmol, 1.0 equiv), fluocinolone (95.1 mg, 0.2 mmol, 0.5 equiv) and magnesium sulfate (96.9 mg, 0.8 mmol, 1.7 equiv) in acetonitrile (2.0 mL) was added trifluoromethanesulfonic acid (103.8 mg, 0.7 mmol, 1.5 equiv) at 0° C. under air atmosphere. The resulting mixture was stirred for 2 h at 0° C. The reaction was monitored by LCMS. The mixture was basified to pH 7 with sodium bicarbonate(aq.). The resulting mixture was filtered, the filter cake was washed with acetonitrile (3×10.0 mL). The filtrate was concentrated under reduced pressure. The crude product was purified by reverse phase flash with the following conditions (column, C₁₈ silica gel; mobile phase, acetonitrile in water (0.1% trifluoroacetic acid), 10% to 100% gradient in 20 min; detector, UV 254 nm.) to afford (2S,6aS,6bR,7S,8aS,8bS,10R,11aR,12aS,12bS)-2,6b-difluoro-10-(4-fluorothiophen-3-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (11.7 mg, 9.9%) as a white solid: MS m/z [M+H]⁺ (ESI): 525.30; ¹H NMR (300 MHz, Methanol-d₄) δ: 0.97 (s, 3H), 1.57 (s, 3H), 1.62-1.81 (m, 4H), 2.23-2.48 (m, 3H), 2.62-2.71 (m, 1H), 4.28-4.36 (m, 2H), 4.63 (d, J=19.5 Hz, 1H), 5.04-5.06 (m, 1H), 5.46-5.62 (m, 2H), 6.31-6.35 (m, 2H), 6.91-6.93 (m, 1H), 7.31 (d, J=9.9 Hz, 1H), 7.48-7.49 (m, 1H).

### Preparation of (4-fluorothiophen-3-yl)methanol

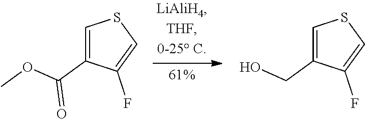

To a stirred solution of methyl 4-fluorothiophene-3-carboxylate (200.0 mg, 1.2 mmol, 1.0 equiv) in tetrahydrofuran (1.0 mL) was added lithium aluminium hydride (71.4 mg, 1.9 mmol, 1.5 equiv) at 0° C. under nitrogen atmosphere. The resulting mixture was stirred for 3 h at 25° C. under nitrogen atmosphere. The reaction was monitored by TLC. The resulting mixture was diluted with methylene chloride (20 mL). The reaction was quenched by the addition of water (20.0 mL) at 0° C. The resulting mixture was extracted with methylene chloride (3×20.0 mL). The combined organic layers were dried over anhydrous sodium sulfate. After filtration, the filtrate was concentrated under reduced pressure to afford (4-fluorothiophen-3-yl)methanol (100.7 mg, 60.6%) as a light yellow oil.

### Preparation of 4-fluorothiophene-3-carbaldehyde

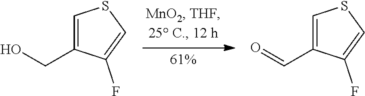 [SMILES: OCc1cscc1F ; O=Cc1cscc1F]

To a stirred solution of (4-fluorothiophen-3-yl)methanol (100.0 mg), 0.8 mmol, 1.0 equiv) in methylene chloride (2.0 mL) was added manganese dioxide (657.9 mg, 7.6 mmol, 10 equiv) at room temperature. The resulting mixture was stirred for 12 h at room temperature. The precipitated solids were collected by filtration and washed with methylene chloride (3×10.0 mL). The resulting mixture was concentrated under vacuum to afford 4-fluorothiophene-3-carbaldehyde (60.0 mg, 60.9%) as a yellow oil.

### Example 30. Preparation of (2S,6aS,6bR,7S,8aS,8bS,10R,11aR,12aS,12bS)-10-(2,3-difluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

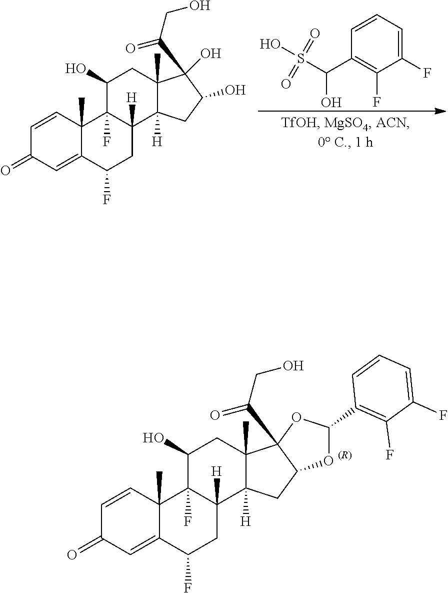

To a solution of fluocinolone (107 mg, 260 μmol, 1 eq) in CH₃CN (2 mL) was added MgSO₄ (94.0 mg, 780 μmol, 3 eq), (2,3-difluorophenyl)-hydroxy-methanesulfonic acid (70 mg, 312 μmol, 1.2 eq) and TfOH (195 mg, 1.30 mmol, 115 uL, 5 eq) at 0° C. The mixture was stirred at 20° C. for 1 hr. The pH of the reaction mixture was adjusted to ˜7 with NaHCO₃(aq) at 0° C. and then diluted with H₂O (4 mL) and extracted with DCM (3 mL×3). The combined organic layers were washed with brine (3 mL×2), dried over Na₂SO₄, filtered, and concentrated under reduced pressure to give a residue. The residue was purified by prep-HPLC (TFA condition; column: Phenomenex C₁₈ 80×30 mm×3 um; mobile phase: [water(TFA)-ACN]; B %: 30%-50%, 8 min) to give (2S,6aS,6bR,7S,8aS,8bS,10R,11aR,12aS,12bS)-10-(2,3-difluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (49.8 mg, 92.9 μmol, 35.7% yield) was obtained as a white solid: MS m/z [M+H]⁺ (ESI): 537.20; ¹H NMR (400 MHz, Methanol-d₄) δ: 7.39-7.35 (m, 1H), 7.34-7.30 (m, 2H), 7.23-7.17 (m, 1H), 6.34-6.31 (m, 2H), 5.80 (s, 1H), 5.63-5.47 (m, 1H), 5.11 (d, J=4.4 Hz, 1H), 4.65 (d, J=19.6 Hz, 1H), 4.37-4.29 (m, 2H), 2.77-2.63 (m, 1H), 2.39-2.31 (m, 2H), 2.26-2.21 (m, 1H), 1.87-1.79 (m, 2H), 1.74-1.70 (m, 1H), 1.62 (d, J=14.0 Hz, 1H), 1.58 (s, 3H), 1.00 (s, 3H).

### Preparation of (2,3-difluorophenyl)-hydroxy-methanesulfonic acid

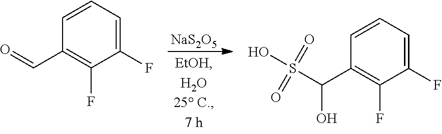

To a solution of 2,3-difluorobenzaldehyde (300 mg, 2.11 mmol, 231 uL, 1 eq) in EtOH (6 mL) was added a solution of sodium metabisulfite (200 mg, 1.06 mmol, 167 uL, 0.5 eq) in H₂O (0.4 mL) at 25° C., and then stirred at 25° C. for 7 hours. The reaction mixture was filtered to give a crude solid product. The crude product was triturated with CH₃CN at 25° C. for 5 min to give (2,3-difluorophenyl)-hydroxy-methanesulfonic acid (70 mg, 312 μmol, 14.8% yield) as a white solid: ¹H NMR (400 MHz, DMSO-d₆) δ: 7.45-7.39 (m, 1H), 7.31-7.21 (m, 1H), 7.16-7.06 (m, 1H), 6.21 (d, J=6.4 Hz, 1H), 5.25 (d, J=6.0 Hz, 1H).

### Example 31. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-methyl-1H-indol-4-yl)-1,2,6a,6b,7,8,8a,8b,11a,12,12a,12b-dodecahydro-4H-naphtho[2′,1′:4,5]indeno[1,2-d][1,3]dioxol-4-one

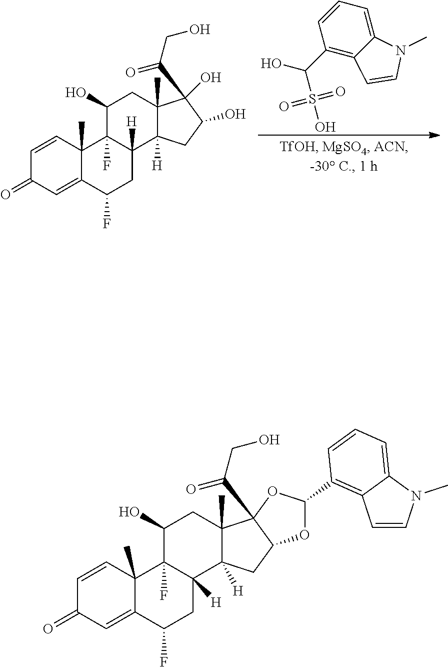

To a solution of fluocinolone (200 mg, 485 μmol, 1 eq) in CH₃CN (8.00 mL) was added MgSO₄ (350 mg, 2.91 mmol, 6 eq) and hydroxy-(1-methylindol-4-yl)methanesulfonic acid (140 mg, 582 μmol, 1.2 eq) and TfOH (362 mg, 2.41 mmol, 213 uL, 5 eq) at −30° C. The mixture was stirred at −30° C. for 1 h. The mixture was quenched until pH=˜7 with NaHCO₃(aq) at 0° C., and then diluted with H₂O (5 ml) and extracted with DCM (20 mL×3). The combined organic layers were washed with brine (3 mL×2), dried over Na₂SO₄, filtered, and concentrated under reduced pressure to give a residue. The residue was purified by prep-HPLC (column: Phenomenex Luna 80×30 mm×3 um; mobile phase: [water(TFA)-ACN]; B %: 35%-60%, 8 min) to give (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-methyl-1H-indol-4-yl)-1,2,6a,6b,7,8,8a,8b,11a,12,12a,12b-dodecahydro-4H-naphtho[2′,1′:4,5]indeno[1,2-d][1,3]dioxol-4-one (15 mg, 22.47 μmol, 4.63% yield, TFA) as gray solid: MS m/z [M+H]⁺ (ESI): 554.20; ¹H NMR (400 MHz, DMSO-d₆) δ: 7.49-7.43 (m, 1H), 7.33 (d, J=3.2 Hz, 1H), 7.25 (d, J=10.4 Hz, 1H), 7.18-7.11 (m, 2H), 6.50 (d, J=2.8 Hz, 1H), 6.28 (dd, J=1.6, 10.4 Hz, 1H), 6.11 (s, 1H), 5.78 (s, 1H), 5.73-5.54 (m, 1H), 5.01 (d, J=4.4 Hz, 1H), 4.54 (d, J=19.2 Hz, 1H), 4.22 (d, J=19.6 Hz, 1H), 4.16 (ddd, J=0.8, 2.8, 7.2 Hz, 1H), 3.75 (s, 3H), 2.56-2.50 (m, 1H), 2.38-2.18 (m, 2H), 2.06-1.95 (m, 1H), 1.79-1.63 (m, 3H), 1.49-1.43 (m, 4H), 0.86 (s, 3H).

### Preparation of hydroxy-(1-methylindol-4 yl)methanesulfonic acid

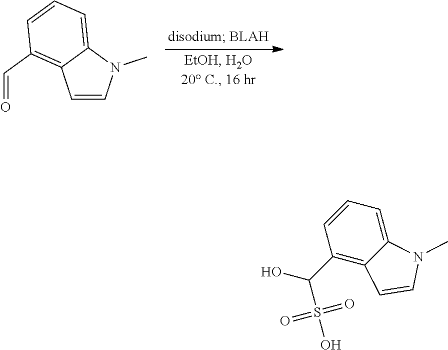 [SMILES: Cn1ccc2c(C=O)cccc21 ; Cn1ccc2c(C(O)S(=O)(=O)O)cccc21]

To a solution of 1-methylindole-4-carbaldehyde (850 mg, 5.34 mmol, 1.00 eq) in EtOH (20.0 mL) was added a solution of disodium; BLAH (1.52 g, 8.01 mmol, 1.27 mL, 1.50 eq) in H₂O (2 mL) at 20° C. The mixture was stirred at 20° C. for 16 hrs. The mixture was filtered, then the cake was dried in vacuum. The crude product was triturated with CH₃CN at 20° C. for 5 min to afford hydroxy-(1-methylindol-4-yl)methanesulfonic acid (1.20 g, 4.97 mmol, 93.15% yield) as white solid: ¹H NMR (400 MHz, DMSO-d₆) δ: 7.28-7.14 (m, 3H), 7.10-7.00 (m, 1H), 6.52 (d, J 3.2 Hz, 1H), 5.52 (d, J=5.2 Hz, 1H), 5.32 (d, J=5.2 Hz, 1H), 3.75 (s, 3H).

### Example 32. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-methyl-1H-indazol-4-yl)-1,2,6a,6b,7,8,8a,8b,11a,12,12a,12b-dodecahydro-4H-naphtho[2′,1′:4,5]indeno[1,2-d][1,3]dioxol-4-one

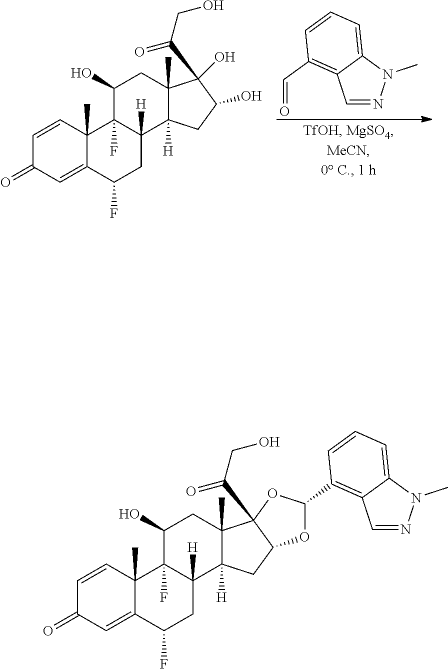

To a solution of fluocinolone (300 mg, 727 μmol, 1.0 eq) in MeCN (6 mL) was added MgSO₄ (306 mg, 2.55 mmol, 3.5 eq) at 0° C. After addition, the mixture was stirred at this temperature for 10 min, and then 1-methylindazole-4-carbaldehyde (175 mg, 1.09 mmol, 1.5 eq), TfOH (328 mg, 2.18 mmol, 193 uL, 3.0 eq) was added at 0° C. The resulting mixture was stirred at 0° C. for 1 hour. The resulting solution was quenched until pH to ˜7 with saturated sodium bicarbonate solution, the mixture was diluted with 10 mL of water and extracted with 3×15 mL of dichloromethane and the organic layers were combined, washed with 10 mL of saturated sodium chloride solution, dried over anhydrous sodium sulfate, filtered, and concentrated under reduced pressure. The residue was purified by prep-HPLC (column: Phenomenex Luna 80×30 mm×3 um; mobile phase: [water (TFA)-ACN]; B %: 30%-55%, 8 min). (2S,6aS,6bR,7S,8aS,8bS,10R,11aR,12aS,12bS)-2,6b-Difluoro-7-hydroxy-8b-(2-hydroxyacetyl)-6a,8a-dimethyl-10-(1-methyl-1H-indazol-4-yl)-1,2,6a,6b,7,8,8a,8b,11a,12,12a,12b-dodecahydro-4H-naphtho[2′,1′:4,5]indeno[1,2-d][1,3]dioxol-4-one (260 mg, 383 μmol, 52.6% yield, 98.5% purity, TFA) was obtained as a white solid: MS m/z [M+H]⁺ (ESI): 555.40; ¹H NMR (400 MHz, DMSO-d₆) δ: 8.13 (s, 1H), 7.59 (d, J=8.4 Hz, 1H), 7.42 (t, J=6.8 Hz, 1H), 7.33-7.25 (m, 2H), 6.35-6.25 (m, 2H), 5.83 (s, 1H), 5.65-5.42 (m, 1H), 5.18 (d, J=2.4 Hz, 1H), 4.71 (d, J=19.2 Hz, 1H), 4.43-4.27 (m, 2H), 4.05 (s, 3H), 2.81-2.59 (m, 1H), 2.51-2.32 (m, 2H), 2.30-2.21 (m, 1H), 1.91-1.80 (m, 2H), 1.77-1.73 (m, 1H), 1.67-1.60 (m, 1H), 1.57 (s, 3H), 1.02 (s, 3H).

### Example 33. Preparation of (2S,6aS,6bR,7S,8aS,8bS,10R,11aR,12aS,12bS)-2,6b-difluoro-10-(3-fluoro-1-methyl-1H-indazol-4-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

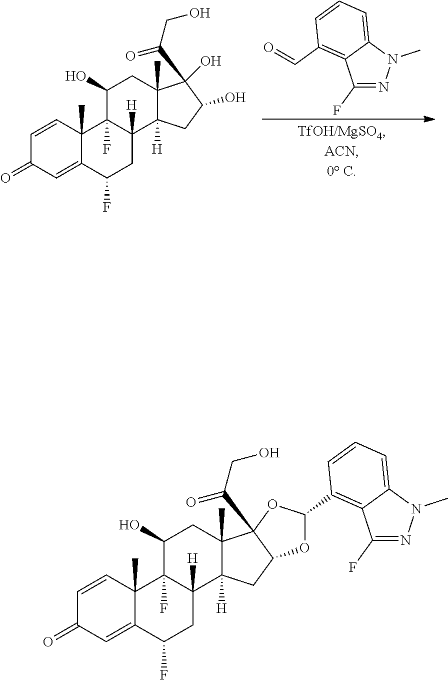

To a mixture of fluocinolone (135 mg, 327 μmol, 1 eq) in MeCN (10 mL) was added MgSO₄ (118 mg, 982 μmol, 3 eq) and 3-fluoro-1-methyl-indazole-4-carbaldehyde (70 mg, 393 μmol, 1.2 eq) and TfOH (172 mg, 1.15 mmol, 101 uL, 3.5 eq) at 0° C., and then stirred at 0° C. for 1 hr. The mixture was quenched by saturated aqueous solution of NaHCO₃ until PH=˜7. The mixture was poured into water (20 mL). The aqueous phase was extracted with DCM (20 mL). The organic layer was dried over Na₂SO₄, concentrated to residue. The crude was purified by Pre_HPLC(TFA)(column: Phenomenex Luna 80×30 mm×3 um; mobile phase: [water(TFA)-ACN]; B %: 35%-60%, 8 min) to give (2S,6aS,6bR,7S,8aS,8bS,10R,11aR,12aS,12bS)-2,6b-difluoro-10-(3-fluoro-1-methyl-1H-indazol-4-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4, 5]indeno[1,2-d][1,3]dioxol-4-one (70 mg, 122 μmol, 37.2% yield, 99.5% purity) as white solid: MS m/z [M+H]⁺ (ESI): 573.20; ¹H NMR (400 MHz, DMSO-d₆) δ: 7.67 (dd, J=2.0, 8.4 Hz, 1H), 7.55-7.47 (m, 1H), 7.35 (d, J=7.2 Hz, 1H), 7.23 (d, J=10.0 Hz, 1H), 6.27 (dd, J=1.6, 10.0 Hz, 1H), 6.10 (s, 1H), 5.93 (s, 1H), 5.75-5.48 (m, 2H), 5.07 (d, J=5.2 Hz, 1H), 4.60 (d, J=19.6 Hz, 1H), 4.26 (d, J=19.6 Hz, 1H), 4.16 (br d, J=8.4 Hz, 1H), 3.91 (s, 3H), 2.72-2.56 (m, 1H), 2.34-1.98 (m, 3H), 1.84-1.65 (m, 3H), 1.50-1.44 (m, 4H), 0.87 (s, 3H).

### Preparation of 4-bromo-3 fluoro-1H-indazole

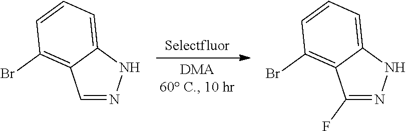 [SMILES: Brc1cccc2[nH]ncc12 ; Fc1n[nH]c2cccc(Br)c12]

1-(Chloromethyl)-4-fluoro-1,4-diazabicyclo[2.2.2]octane-1,4-diium ditetrafluoroborate (Selectfluor™, 9.89 g, 27.91 mmol, 1.1 eq) was added to a solution of 4-bromo-1H-indazole (5 g, 25.4 mmol, 1 eq) in DMA (10 mL) under N2. The mixture was stirred at 60° C. for 10 hr. The reaction mixture was partitioned between EtOAc (20 mL) and water (20 mL). The organic phase was separated, washed with brine (20 mL), dried over Na₂SO₄, filtered, and concentrated under reduced pressure to give a residue. The crude was purified by column chromatography (SiO₂, Petroleum ether/Ethyl acetate=1/0 to 1/1) to give 4-bromo-3-fluoro-1H-indazole (0.83 g, 3.86 mmol, 15.2% yield) as yellow solid: MS m/z [M+H]⁺ (ESI): 214.9; ¹H NMR (400 MHz, DMSO-d₆) δ: 7.51 (dd, J=1.6, 8.4 Hz, 1H), 7.46-7.23 (m, 2H).

### Preparation of 4-bromo-3-fluoro-1-methyl-indazole

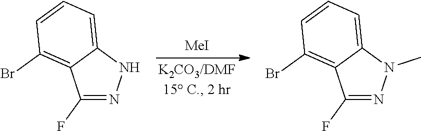

To a mixture of 4-bromo-3-fluoro-1H-indazole (0.82 g, 3.81 mmol, 1 eq) in DMSO (40 mL) was added Cs₂CO₃ (1.49 g, 4.58 mmol, 1.2 eq) and MeI (798 mg, 5.62 mmol, 0.35 mL, 1.47 eq), and then stirred at 15° C. for 2 hr. The reaction mixture was partitioned between EtOAc (100 mL) and water (100 mL). The organic phase was separated, washed with brine (30 mL), dried over Na₂SO₄, filtered, and concentrated under reduced pressure to give a residue. The crude was purified by column chromatography (SiO₂, Petroleum ether/Ethyl acetate=1/0 to 3/1) to give 4-bromo-3-fluoro-1-methyl-indazole (0.6 g, 2.62 mmol, 68.7% yield) as yellow solid: MS m/z [M+H]⁺ (ESI): 228.9; ¹H NMR (400 MHz, DMSO-d₆) δ: 7.52 (td, J=2.4, 6.8 Hz, 1H), 7.39-7.27 (m, 2H), 3.94 (s, 3H).

### Preparation 3-fluoro-1-meth

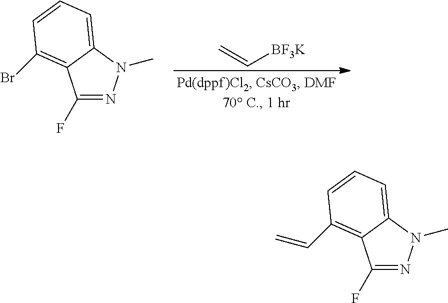

To a solution of 4-bromo-3-fluoro-1-methyl-indazole (0.4 g, 1.75 mmol, 1 eq) and potassium; trifluoro(vinyl)boranuide (468 mg, 3.49 mmol, 2 eq) in DMF (10 mL) was added Cs₂CO₃ (1.71 g, 5.24 mmol, 3 eq) and Pd(dppf)Cl₂ (128 mg, 175 μmol, 0.1 eq). The mixture was stirred at 70° C. for 1 hr. The reaction mixture was partitioned between EtOAc (10 mL) and water (10 mL). The organic phase was separated, washed with brine (5 mL), dried over Na₂SO₄, filtered, and concentrated under reduced pressure to give a residue. The mixture was purified by column chromatography (SiO₂, Petroleum ether/Ethyl acetate=1/0 to 2/1) to give 3-fluoro-1-methyl-4-vinyl-indazole (0.17 g, 965 μmol, 55.3% yield) as yellow solid: MS m/z [M+H]⁺ (ESI): 177.0; ¹H NMR (400 MHz, DMSO-d₆) δ: 7.56 (dd, J=2.0, 8.4 Hz, 1H), 7.50-7.37 (m, 2H), 7.18-7.08 (m, 1H), 6.04 (d, J=17.6 Hz, 1H), 5.52 (d, J=11.2 Hz, 1H), 3.93 (s, 3H).

### Preparation of 3 fluoro-1-methyl-indazole-4-carbaldehyde

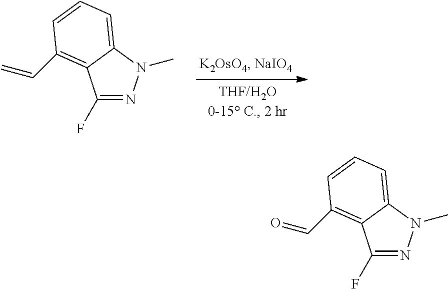

To a mixture of 3-fluoro-1-methyl-4-vinyl-indazole (0.17 g, 965 μmol, 1 eq) in THE (5 mL) and H₂O (1 mL) was added dipotassium; dioxido(dioxo)osmium; dihydrate (35.6 mg, 96.5 μmol, 0.1 eq) and sodium; periodate (1.03 g, 4.82 mmol, 267 uL, 5 eq) at 0° C., and then stirred at 15° C. for 2 hr. The mixture was diluted with EtOAc (20 mL) and water (20 mL). The organic layer was washed by saturated aqueous solution of Na₂S₂O₃ (20 mL). The organic layer was dried over Na₂SO₄, concentrated to residue. The crude was purified by column chromatography (SiO₂, Petroleum ether/Ethyl acetate=1/0 to 3/1) to give 3-fluoro-1-methyl-indazole-4-carbaldehyde (0.08 g, 449 μmol, 46.5% yield) as white solid: MS m/z [M+H]+ (ESI): 179.0; ¹H NMR (400 MHz, DMSO-d₆) δ: 10.25 (s, 1H), 8.06 (br d, J=9.2 Hz, 1H), 7.88 (br d, J=6.4 Hz, 1H), 7.78-7.62 (m, 1H), 4.02 (s, 3H).

### Example 34. Preparation of (2S,6aS,6bR,7S,8aS,8bS,10R,11aR,12aS,12bS)-10-(3-ethoxy-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

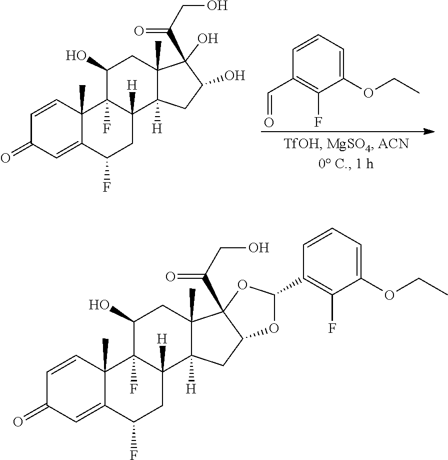

To a solution of fluocinolone (307 mg, 743 μmol, 1 eq) in CH₃CN (6 mL) was added MgSO₄ (268 mg, 2.23 mmol, 3 eq), 3-ethoxy-2-fluoro-benzaldehyde (150 mg, 892 μmol, 1.2 eq) and trifluoromethanesulfonic acid (558 g, 3.72 mmol, 328 uL, 5 eq) at 0° C. The mixture was stirred at 0° C. for 1 hr under N2 atmosphere. The reaction mixture was quenched until pH=˜7 with NaHCO₃(aq) at 0° C. and then diluted with H₂O (5 m1) and extracted with DCM (20 mL×3). The combined organic layers were washed with brine (3 mL×2), dried over Na₂SO₄, filtered, and concentrated under reduced pressure to give a residue. The residue was purified by prep-HPLC (TFA condition; column: Phenomenex Luna 80×30 mm×3 um; mobile phase: [water(TFA)-ACN]; B %: 40%-60%, 8 min) to give (2S,6aS,6bR,7S,8aS,8bS,10R,11aR,12aS,12bS)-10-(3-ethoxy-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (147 mg, 261.30 μmol, 35.15% yield) as a white solid: MS m/z [M+H]⁺ (ESI): 563.20; ¹H NMR (400 MHz, DMSO-d₆) δ: 7.26 (d, J=10.0 Hz, 1H), 7.20-7.12 (m, 2H), 7.05-7.01 (m, 1H), 6.31-6.28 (m, 1H), 6.12 (s, 1H), 5.71-5.54 (m, 2H), 4.98 (d, J=3.6 Hz, 1H), 4.50 (d, J=19.2 Hz, 1H), 4.24-4.16 (m, 2H), 4.10-4.05 (m, 2H), 2.70-2.56 (m, 1H), 2.31-2.28 (m, 1H), 2.22-2.15 (m, 1H), 2.05-2.01 (m, 1H), 1.73-1.69 (m, 3H), 1.51-1.39 (m, 4H), 1.31 (t, J=7.6 Hz, 3H), 0.86 (s, 3H).

### Preparation of 3-ethoxy-2 fluoro-benzaldehyde

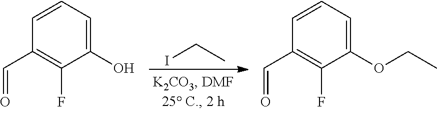

To a solution of 2-fluoro-3-hydroxy-benzaldehyde (200 mg, 1.43 mmol, 1 eq) in DMF (4 mL) was added K₂CO₃ (395 mg, 2.85 mmol, 2 eq) and iodoethane (445 mg, 2.85 mmol, 228 uL, 2 eq), and then stirred at 25° C. for 2 hr. The reaction mixture was quenched by addition H₂O (20 mL) at 0° C., and extracted with EtOAc (20 mL×3). The combined organic layers were washed with brine (10 mL×2), dried over Na₂SO₄, filtered, and concentrated under reduced pressure to give 3-ethoxy-2-fluoro-benzaldehyde (220 mg, 1.31 mmol, 91.7% yield) as yellow oil; MS m/z [M+H]⁺ (ESI): 169.0; ¹H NMR (400 MHz, DMSO-d₆) δ: 10.21 (s, 1H), 7.51-7.45 (m, 1H), 7.36-7.30 (m, 1H), 7.27 (t, J=15.6 Hz, 1H), 4.19-4.09 (m, 2H), 1.35 (t, J=7.6 Hz, 3H).

### Example 35. Preparation of (2S,6aS,6bR,7S,8aS,8bS,10R,11aR,12aS,12bS)-10-(3-ethyl-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

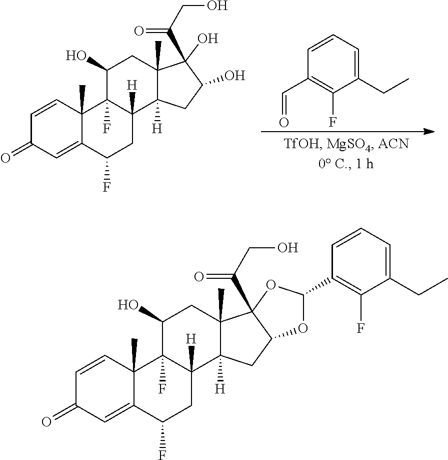

To a solution of fluocinolone (72.3 mg, 175 μmol, 1 eq) in MeCN (5 mL) was added MgSO₄ (73.8 mg, 613 μmol, 3.5 eq), 3-ethyl-2-fluoro-benzaldehyde (0.04 g, 263 μmol, 1.5 eq) followed by the addition of trifluoromethanesulfonic acid (78.9 mg, 526 μmol, 46.4 uL, 3 eq) at 0° C. The resulting solution was stirred for 1 h at 0° C. with an inert atmosphere of nitrogen. The mixture was quenched by saturated aqueous solution of NaHCO₃ until PH=˜7. The mixture was diluted with water (20 mL), extracted with DCM (20 mL). The organic layer was dried over Na₂SO₄, concentrated to give a residue. The crude was purified by Pre_HPLC (column: Phenomenex Luna 80×30 mm×3 um; mobile phase: [water(TFA)-ACN]; B %: 45%-65%, 8 min) to give (2S,6aS,6bR,7S,8aS,8bS,10R,11aR,12aS,12bS)-10-(3-ethyl-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (43 mg, 76.6 μmol, 43.7% yield, 97.4% purity): MS m/z [M+H]⁺ (ESI): 547.30; ¹H NMR (400 MHz, DMSO-d₆) δ: 7.41 7.10 (m, 4H), 6.29 (dd, J=1.6, 10.4 Hz, 1H), 6.11 (s, 1H), 5.77-5.21 (m, 6H), 4.99 (d, J=5.2 Hz, 1H), 4.51 (d, J=19.6 Hz, 1H), 4.23-4.15 (m, 2H), 2.65-2.59 (m, 3H), 2.36-1.97 (m, 3H), 1.75-1.70 (m, 3H), 1.52-1.47 (m, 4H), 1.15 (t, J=7.6 Hz, 3H), 0.87 (s, 3H).

### Preparation of 3-ethyl-2 fluoro-benzaldehyde

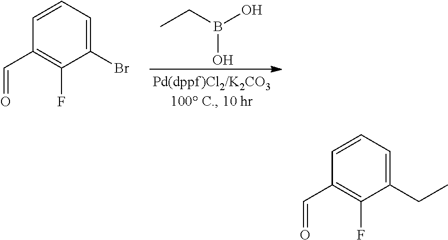

To a solution of 3-bromo-2-fluoro-benzaldehyde (0.5 g, 2.46 mmol, 1 eq) and Cs₂CO₃ (1.60 g, 4.93 mmol, 2 eq) in toluene (15 mL) and Water (0.5 mL) was added ethyl-trifluoro-potassio-boron (1−) (670 mg, 4.93 mmol, 2 eq) and Pd(dppf)Cl₂ (180 mg, 246 μmol, 0.1 eq). The mixture was stirred at 100° C. for 10 hr under N2. The reaction mixture was concentrated to give a residue. The residue was diluted in EtOAc (20 ml) and water (20 mL). The organic layer was dried over Na₂SO₄, concentrated. The crude was purified by column chromatography to give 3-ethyl-2-fluoro-benzaldehyde (0.05 g, 329 μmol, 13.3% yield) as yellow oil: ¹H NMR (400 MHz, CDCl₃) δ: 10.31 (s, 1H), 7.64-7.61 (m, 1H), 7.40 (t, J=7.2 Hz, 1H), 7.10 (t, J=7.2 Hz, 1H), 2.67 (q, J=7.6 Hz, 2H), 1.20 (t, J=7.6 Hz, 3H).

### Example 36. Preparation of (2S,6aS,6bR,7S,8aS,8bS,10R,11aR,12aS,12bS)-10-(3-(difluoromethoxy)-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

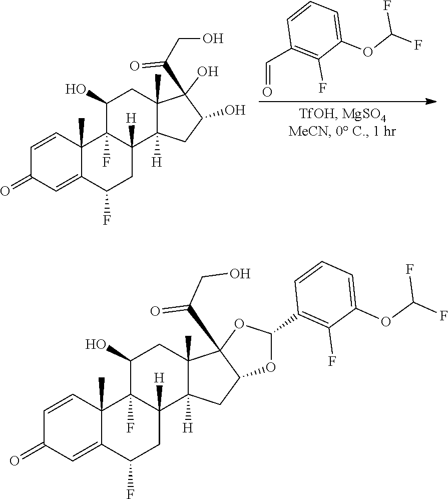

To a solution of fluocinolone (260 mg, 631 μmol, 1.0 eq) and MgSO₄ (266 mg, 2.21 mmol, 3.5 eq) in MeCN (2 mL) was added 3-(difluoromethoxy)-2-fluoro-benzaldehyde (180 mg, 947 μmol, 1.5 eq) and trifluoromethanesulfonic acid (289 mg, 1.93 mmol, 170 uL, 3.05 eq) at 0° C. The mixture was stirred at 0° C. for 1 hour. The reaction mixture was quenched until pH=7 with NaHCO₃(aq) at 0° C., and then diluted with H₂O (5 ml) and extracted with DCM (20 mL×3). The combined organic layers were washed with brine (20 mL×2), dried over Na₂SO₄, filtered, and concentrated under reduced pressure to give a residue. The residue was purified by prep-HPLC (column: Phenomenex Luna 80×30 mm×3 um; mobile phase: [water(TFA)-ACN]; B %: 40%-60%, 8 min). (2S,6aS,6bR,7S,8aS,8bS,10R,11aR,12aS,12bS)-10-(3-(difluoromethoxy)-2-fluorophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4, 5]indeno[1,2-d][1,3]dioxol-4-one (171 mg, 292.54 μmol, 46.35% yield) was obtained as a white solid: MS m/z [M+H]⁺ (ESI): 585.20; ¹H NMR (400 MHz, Methanol-d₄) δ: 7.48-7.45 (m, 1H), 7.37-7.29 (m, 2H), 7.26-7.20 (m, 1H), 7.02-6.65 (m, 1H), 6.35-6.29 (m, 2H), 5.81 (s, 1H), 5.66-5.46 (m, 1H), 5.11 (d, J=4.4 Hz, 1H), 4.69-4.32 (m, 2H), 4.32-4.28 (m, 1H), 2.79-2.62 (m, 1H), 2.41-2.30 (m, 2H), 2.23-2.21 (m, 1H), 1.88-1.60 (m, 4H), 1.58 (s, 3H), 1.00 (s, 3H).

### Preparation of 3-(difluoromethoxy)-2 fluoro-benzaldehyde

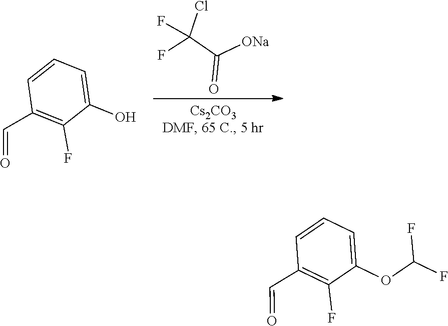

To a solution of 2-fluoro-3-hydroxy-benzaldehyde (400 mg, 2.85 mmol, 1.0 eq) in DMF (8 mL) was added (2-chloro-2,2-difluoro-acetyl)oxysodium (1.09 g, 7.14 mmol, 2.5 eq) and Cs₂CO₃ (3.26 g, 9.99 mmol, 3.5 eq). The mixture was stirred at 65° C. for 5 hours. The reaction mixture was quenched by addition H₂O 20 mL at 0° C., and then extracted with EtOAc (10 mL×3). The combined organic layers were washed with brine 10 mL, dried over Na₂SO₄, filtered, and concentrated under reduced pressure to give a residue. The residue was purified by flash silica gel chromatography (Biotage®; 12 g SepaFlash® Silica Flash Column, Eluent of 0-20% Ethyl acetate/petroleum ether gradient @ 40 mL/min). 3-(difluoromethoxy)-2-fluoro-benzaldehyde (170 mg, 894.18 μmol, 31.32% yield) was obtained as colorless oil.

### Example 37. Preparation of (2S,6aS,6bR,7S,8aS,8bS,10R,11aR,12aS,12bS)-2,6b-difluoro-10-(2-fluoro-3-(2,2,2-trifluoroethoxy)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

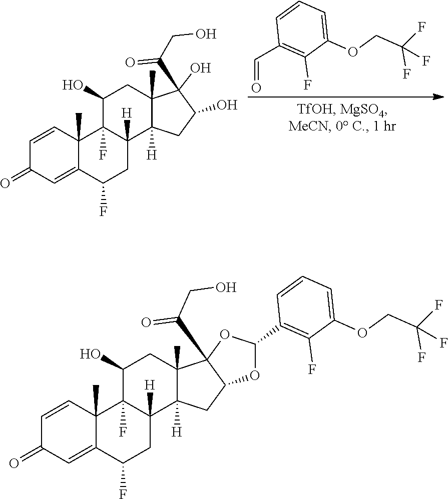

To a solution of fluocinolone (300 mg, 72 7 μmol, 1.0 eq), MgSO₄ (306 mg, 2.55 mmol, 3.5 eq) in MeCN (3 mL) was added 2-fluoro-3-(2,2,2-trifluoroethoxy)benzaldehyde (242 mg, 1.09 mmol, 1.5 eq) and trifluoromethanesulfonic acid (340 mg, 2.27 mmol, 200 uL, 3.11 eq) at 0° C. The mixture was stirred at 0° C. for 1 hour. The reaction mixture was quenched until pH=7 with NaHCO₃(aq) at 0° C., and then diluted with H₂O (5 ml) and extracted with DCM (20 mL×3). The combined organic layers were washed with brine (20 mL×2), dried over Na₂SO₄, filtered, and concentrated under reduced pressure to give a residue. The residue was purified by prep-HPLC (column: Phenomenex Luna 80×30 mm×3 um; mobile phase: [water(TFA)-ACN]; B %: 40%-60%, 8 min). (2S,6aS,6bR,7S,8aS,8bS,10R,11aR,12aS,12bS)-2,6b-difluoro-10-(2-fluoro-3-(2,2,2-trifluoroethoxy)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (153.4 mg, 248.81 μmol, 34.20% yield) was obtained as a white solid: MS m/z [M+H]⁺ (ESI): 617.20; ¹H NMR (400 MHz, Methanol-d₄) δ: 7.33-7.24 (m, 2H), 7.23-7.13 (m, 2H), 6.35-6.29 (m, 2H), 5.80 (s, 1H), 5.64-5.44 (m, 1H), 5.09 (d, J=4.8 Hz, 1H), 4.69-4.54 (m, 3H), 4.38-4.27 (m, 2H), 2.77-2.60 (m, 1H), 2.40-2.19 (m, 3H), 1.87-1.60 (m, 4H), 1.57 (s, 3H), 0.99 (s, 3H).

### Preparation of 2 fluoro-3-(2,2,2-trifluoroethoxy)benzaldehyde

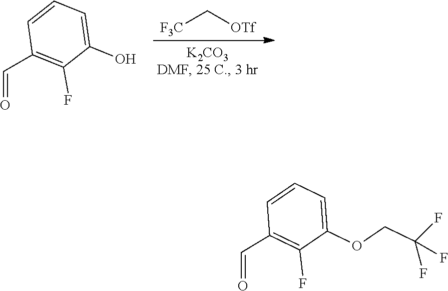 [SMILES: O=Cc1cccc(O)c1F ; O=S(=O)(OCC(F)(F)F)C(F)(F)F ; O=Cc1cccc(OCC(F)(F)F)c1F]

To a solution of 2,2,2-trifluoroethyl trifluoromethanesulfonate (994 mg, 4.28 mmol, 1.5 eq) in DMF (5 mL) was added 2-fluoro-3-hydroxy-benzaldehyde (0.40 g, 2.85 mmol, 1.0 eq) and K₂CO₃ (986 mg, 7.14 mmol, 2.5 eq), and then stirred at 25° C. for 3 hours. The reaction mixture was quenched by addition H₂O (20 mL) at 0° C., and then extracted with EtOAc (15 mL×3). The combined organic layers were washed with brine (10 mL) dried over Na₂SO₄, filtered, and concentrated under reduced pressure to give a residue. The residue was purified by flash silica gel chromatography (Biotage®; 20 g SepaFlash® Silica Flash Column, Eluent of 0-20% Ethyl acetate/petroleum ether gradient @ 60 mL/min). 2-fluoro-3-(2,2,2-trifluoroethoxy)benzaldehyde (600 mg, 2.70 mmol, 94.61% yield) was obtained as a white solid.

### Example 38. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(phenylethynyl)-1,2,6a,6b,7,8,8a,8b,11a,12,12a,12b-dodecahydro-4H-naphtho[2′,1′:4,5]indeno[1,2-d][1,3]dioxol-4-one

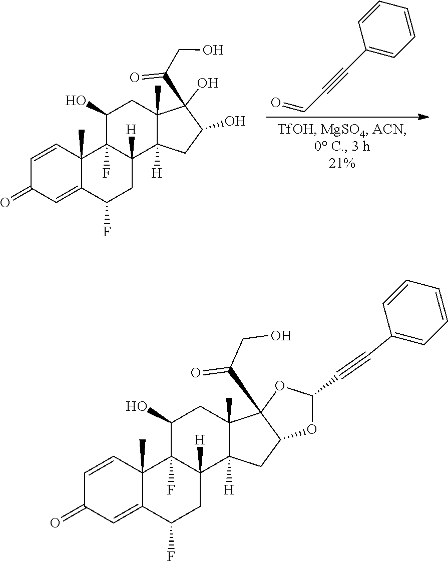

To a solution of fluocinolone (50.0 mg, 0.12 mmol, 1.0 equiv) and 3-phenylprop-2-ynal (17.4 mg, 0.13 mmol, 1.1 equiv) in acetonitrile (1 mL) were added magnesium sulfate (51.1 mg, 0.42 mmol, 3.5 equiv) and trifluoromethanesulfonic acid (54.6 mg, 0.36 mmol, 3.0 equiv) dropwise at 0° C. under nitrogen atmosphere. After completion of reaction. The mixture was neutralized to pH 7 with saturated sodium bicarbonate. The resulting mixture was extracted with ethyl acetate (3×30 mL). The combined organic layers were washed with brine (3×30 mL), dried over anhydrous magnesium sulfate. After filtration, the filtrate was concentrated under reduced pressure. The crude product (40.0 mg) was purified by Prep-HPLC with the following conditions (Column: XBridge Prep OBD C₁₈ Column, 30×150 mm, 5 μm; Mobile Phase A: water (10 mmol/L ammonium bicarbonate), Mobile Phase B: acetonitrile; Flow rate: 60 mL/min; Gradient: 5% B to 30% B in 9 min, 30% B; Wave Length: 254 nm; RT1(min): 7; Number Of Runs: 0) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(phenylethynyl)-1,2,6a,6b,7,8,8a,8b,11a,12,12a,12b-dodecahydro-4H-naphtho[2′,1′:4,5]indeno[1,2-d][1,3]dioxol-4-one (13.5 mg, 21%) as a white solid: MS m/z [M+H]⁺ (ESI): 525.15; ¹H NMR (300 MHz, Methanol-d₄) δ: 0.98 (s, 3H), 1.57 (s, 3H), 1.62-1.82 (m, 4H), 2.27-2.32 (m, 2H), 2.53-2.61 (m, 2H), 4.28-4.35 (m, 2H), 4.62 (d, J=19.2 Hz, 1H), 5.08-5.09 (m, 1H), 5.50-5.66 (m, 2H), 6.29-6.36 (m, 2H), 7.28-7.42 (m, 6H).

### Example 39. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(2-oxaspiro[3.3]heptan-6-yl)-1,2,6a,6b,7,8,8a,8b,11a,12,12a,12b-dodecahydro-4H-naphtho[2′,1′:4,5]indeno[1,2-d][1,3]dioxol-4-one

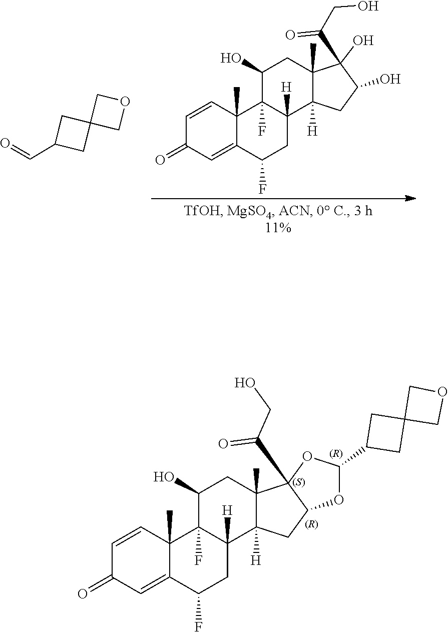

To a solution of 2-oxaspiro[3.3]heptane-6-carbaldehyde (20.0 mg, 0.159 mmol, 1.0 equiv.) in acetonitrile (2.0 mL) with an inert atmosphere of nitrogen, was added fluocinolone (65.4 mg, 0.159 mmol, 1.0 equiv.) and magnesium sulfate (66.8 mg, 0.556 mmol, 3.5 equiv.) for 3 min at 0° C., followed by the addition of trifluoromethanesulfonic acid (71.4 mg, 0.477 mmol, 3.0 equiv.) at 0° C. The resulting solution was stirred for 3 h at 0° C., adjusted pH to 7 with saturated sodium bicarbonate solution (2.0 mL). The mixture was diluted with water (5 mL) and extracted with dichloromethane (3×20 mL) and the organic layers were combined, washed with saturated sodium chloride solution (3×20 mL), dried over anhydrous sodium sulfate, filtered, and concentrated under reduced pressure. The crude product was purified by Flash-Prep-HPLC with the following conditions (IntelFlash-1): Column, GreenSep Naphthyl 120A, 4.6×100 mm, 3 um; Mobile Phase A: CO₂, Mobile Phase B: methanol (1% 2 mol/L ammonia in methanol); Flow rate: 2 mL/min; Gradient: isocratic 5% B; Detector: 220 nm. 2.9 mg (11%) of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(2-oxaspiro[3.3]heptan-6-yl)-1,2,6a,6b,7,8,8a,8b,11a,12,12a,12b-dodecahydro-4H-naphtho[2′,1′:4,5]indeno[1,2-d][1,3]dioxol-4-one was obtained as white solid: MS m/z [M+H]⁺ (ESI): 521.25; ¹H NMR (400 MHz, Methanol-d₄) δ: 0.98 (s, 3H), 1.20-1.28 (m, 1H), 1.48-1.53 (m, 2H), 1.54-1.55 (m, 3H), 1.56-1.68 (m, 3H), 2.16-2.34 (m, 6H), 2.62-2.71 (m, 1H), 3.76-3.88 (m, 4H), 4.24-4.31 (m, 2H), 4.36 (s, 1H), 4.46-4.59 (m, 1H), 4.87-4.93 (m, 1H), 5.46-5.59 (m, 1H), 6.30-6.35 (m, 2H), 7.30-7.32 (m, 1H).

### Preparation of 2-oxaspiro[3.3]heptane-6-carbaldehyde

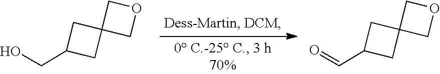 [SMILES: OCC1CC2(COC2)C1 ; O=CC1CC2(COC2)C1]

To a solution of 2-oxaspiro[3.3]heptan-6-ylmethanol (20.0 mg, 0.156 mmol, 1 equiv.) in dichloromethane (2.0 mL) with an inert atmosphere of nitrogen, was added Dess-Martin (132.4 mg, 0.31 mmol, 2 equiv.) at 0° C. The resulting solution was stirred for 3 h at 25° C., diluted with saturated sodium bicarbonate solution (5.0 mL), extracted with dichloromethane (3×20 mL) and organic layers were washed with saturated sodium chloride solution (3×20 mL), dried over anhydrous sodium sulfate, filtration and concentrated under reduced pressure. The residue was purified by silica gel column chromatography, eluted with petroleum ether/ethyl acetate (10:1). 20 mg (70%) of 2-oxaspiro[3.3]heptane-6-carbaldehyde was obtained as a white solid.

### Example 40. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(7-oxaspiro[3.5]nonan-2-yl)-1,2,6a,6b,7,8,8a,8b,11a,12,12a,12b-dodecahydro-4H-naphtho[2′,1′:4,5]indeno[1,2-d][1,3]dioxol-4-one

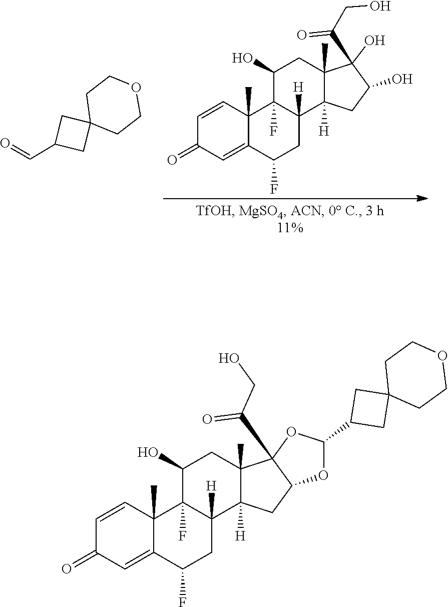

To a stirred solution of 7-oxaspiro[3.5]nonane-2-carbaldehyde (50.0 mg, 0.32 mmol, 1.0 equiv) and fluocinolone (200.5 mg, 0.48 mmol, 1.5 equiv) in acetonitrile was added magnesium sulfate (136.5 mg, 1.13 mmol, 3.5 equiv) dropwise at room temperature under air atmosphere. To the above mixture was added trifluoromethanesulfonic acid (145.9 mg, 0.97 mmol, 3.0 equiv) dropwise over 1 min at 0° C. The resulting mixture was stirred for additional 3 h at room 0° C. The mixture was basified to pH 7 with saturated Sodium bicarbonate (aq.). The crude product was purified by Prep-HPLC with the following conditions (column, C₁₈ silica gel; mobile phase, acetonitrile in water (0.1% ammonia+10 mmol/L ammonium bicarbonate) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(7-oxaspiro[3.5]nonan-2-yl)-1,2,6a,6b,7,8,8a,8b,11a,12,12a,12b-dodecahydro-4H-naphtho[2′,1′:4,5]indeno[1,2-d][1,3]dioxol-4-one (20.8 mg, 11%) as a white solid: MS m/z [M+H]+ (ESI): 549.35; ¹H NMR (400 MHz, Methanol-d₄) δ: 0.93 (s, 3H), 1.50-1.53 (m, 2H), 1.57-1.66 (m, 9H), 1.70-1.85 (m, 4H), 2.21-2.26 (m, 3H), 2.57-2.70 (m, 2H), 3.47-3.49 (m, 2H), 3.57-3.59 (m, 2H), 4.26-4.31 (m, 2H), 4.51-4.59 (m, 2H), 4.91-4.92 (m, 1H), 4.50-4.60 (m, 1H), 6.30-6.35 (m, 2H), 7.30-7.33 (m, 1H).

### Example 41. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(4,5,6,7-tetrahydrobenzo[b]thiophen-2-yl)-1,2,6a,6b,7,8,8a,8b,11a,12,12a,12b-dodecahydro-4H-naphtho[2′,1′:4,5]indeno[1,2-d][1,3]dioxol-4-one

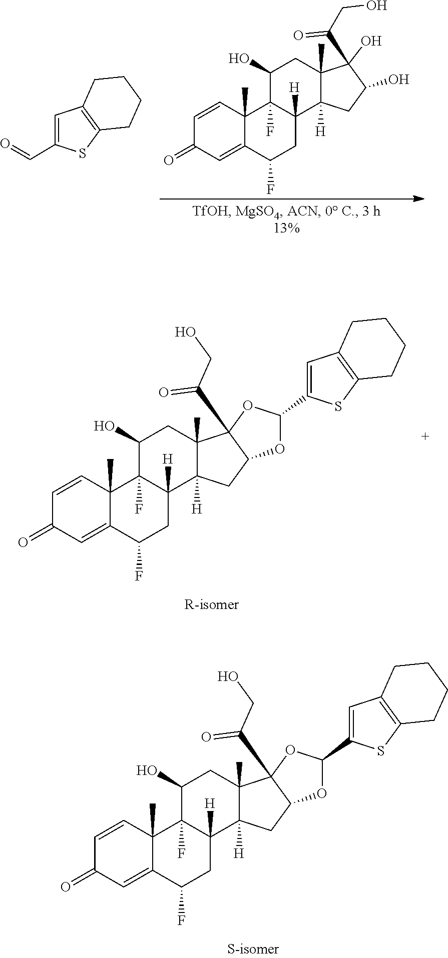

To a stirred solution of 4,5,6,7-tetrahydro-1-benzothiophene-2-carbaldehyde (50.0 mg, 0.30 mmol, 1.5 equiv), magnesium sulfate (84.5 mg, 0.70 mmol, 3.5 equiv) and fluocinolone (82.7 mg, 0.2 mmol, 1.2 equiv) in acetonitrile (5 mL) was added trifluoroacetic acid (68.6 mg, 0.6 mmol, 3 equiv) at 0° C. under air atmosphere. The resulting mixture was stirred for 3 h at 0° C. under. The reaction was monitored by LCMS. The residue was neutralized to pH 7 with saturated sodium bicarbonate (aq.). The resulting mixture was extracted with methylene chloride (3×20 mL). The combined organic layers were dried over anhydrous sodium sulfate. After filtration, the filtrate was concentrated under reduced pressure. The crude product was purified by reverse phase flash with the following conditions (column, C₁₈ silica gel; mobile phase, acetonitrile in water (0.1% trifluoroacetic acid), 10% to 100% gradient in 20 min; detector, UV 254 nm.) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(4,5,6,7-tetrahydrobenzo[b]thiophen-2-yl)-1,2,6a,6b,7,8,8a,8b,11a,12,12a,12b-dodecahydro-4H-naphtho[2′,1′:4,5]indeno[1,2-d][1,3]dioxol-4-one (15.5 mg, 12.7%) as a white solid: MS m/z [M+H]⁺ (ESI): 561.20; ¹H NMR (300 MHz, Methanol-d₄) δ: 1.00 (s, 3H), 1.58 (s, 3H), 1.65-1.82 (m, 8H), 2.23-2.37 (m, 3H), 2.54-2.56 (m, 2H), 2.69-2.73 (m, 3H), 4.27-4.33 (m, 2H), 4.61 (d, J=19.5 Hz, 1H), 5.01-5.02 (m, 1H), 5.50-5.73 (m, 2H), 6.33-6.36 (m, 2H), 6.79 (s, 1H), 7.31-7.34 (m, 1H).

### Example 42. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(4,5,6,7-tetrahydrobenzo[b]thiophen-3-yl)-1,2,6a,6b,7,8,8a,8b,11a,12,12a,12b-dodecahydro-4H-naphtho[2′,1′:4,5]indeno[1,2-d][1,3]dioxol-4-one

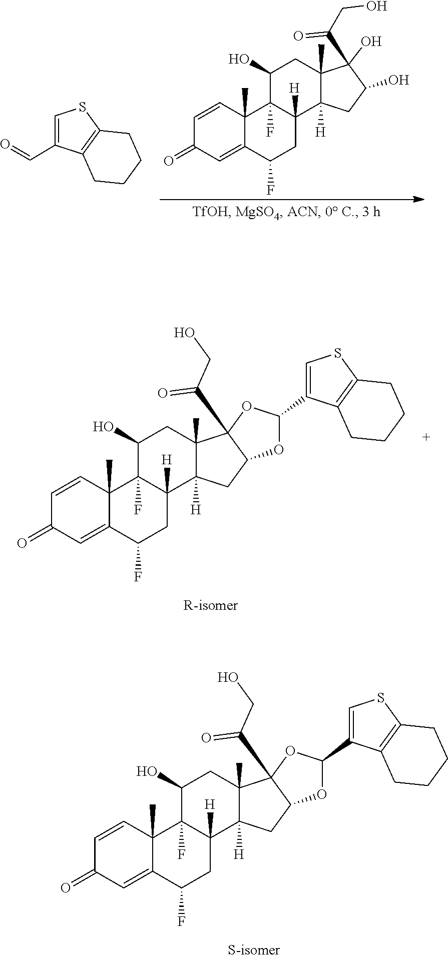

To a stirred solution of 4,5,6,7-tetrahydro-1-benzothiophene-3-carbaldehyde (50 mg, 0.301 mmol, 1 equiv) and fluocinolone (148.86 mg, 0.361 mmol, 1.2 equiv) in acetone were added trifluoromethanesulfonic acid (135.42 mg, 0.903 mmol, 3 equiv) and magnesium sulfate (126.70 mg, 1.053 mmol, 3.5 equiv) at 0° C. under nitrogen atmosphere. The residue product was purified by reverse phase flash with the following conditions (acetonitrile/water with 0.05% ammonium bicarbonate) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(4, 5, 6,7-tetrahydrobenzo[b]thiophen-3-yl)-1,2,6a,6b,7,8,8a,8b, 11a,12,12a,12b-dodecahydro-4H-naphtho[2′,1′:4,5]indeno[1,2-d][1,3]dioxol-4-one (15.5 mg, 8.89%) as a white solid: MS m/z [M+H]⁺ (ESI): 561.25; ¹H NMR (300 MHz, Methanol-d₄) δ 7.32 (dd, J=10.0, 1.5 Hz, 1H), 7.23 (s, 1H), 6.37-6.26 (m, 2H), 5.68-5.39 (m, 2H), 5.06-4.96 (m, 1H), 4.62 (d, J=19.4 Hz, 1H), 4.40-4.23 (m, 2H), 2.80-2.56 (m, 5H), 2.48-2.12 (m, 3H), 1.89-1.65 (m, 7H), 1.57 (s, 4H), 0.97 (s, 3H).

### Example 43. Preparation of methyl (2S,6aS,6bR,7S,8aS,8bS,10R,11aR,12aS,12bS)-2,6b-difluoro-10-((E)-3-fluorostyryl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

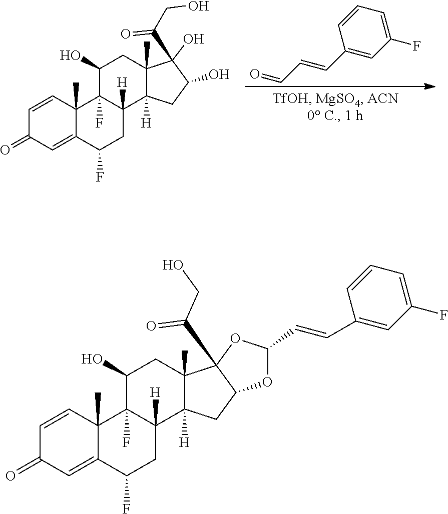

To a mixture of fluocinolone (0.1 g, 242 μmol, 1 eq) and MgSO₄ (116 mg, 969 μmol, 4 eq) in CH₃CN (5 mL) was added (E)-3-(3-fluorophenyl)prop-2-enal (54.6 mg, 363 μmol, 1.5 eq) and trifluoromethanesulfonic acid (109 mg, 727 μmol, 64.22 uL, 3 eq) dropwise at 0° C. under N2, and then stirred at 0° C. for 1 hour. The mixture was quenched with sat. aq. NaHCO₃ until pH to ˜7. The aqueous phase was extracted with ethyl acetate (15 mL×3). The combined organic phase was washed with brine (10 mL), dried with anhydrous Na₂SO₄, filtered, and concentrated in vacuum. The residue was purified by prep-HPLC (column: Phenomenex Luna 80×30 mm×3 um; mobile phase: [water(TFA)-ACN]; B %: 40%-60%, 8 min). (2S,6aS,6bR,7S,8aS,8bS,10R,11aR,12aS,12bS)-2,6b-difluoro-10-((E)-3-fluorostyryl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (32.03 mg, 58.60 μmol, 24.17% yield, 99.63% purity) was obtained as an off-white solid: MS m/z [M+H]⁺ (ESI): 545.3; ¹H NMR (400 MHz, DMSO-d₆) δ: 7.38-7.23 (m, 4H), 7.10 (dt, J=2.0, 8.4 Hz, 1H), 6.82 (d, J=16.0 Hz, 1H), 6.29 (dd, J=2.4, 10.4 Hz, 1H), 6.20 (dd, J=6.0, 16.0 Hz, 1H), 6.12 (s, 1H), 5.71-5.51 (m, 1H), 5.11 (d, J=6.0 Hz, 1H), 4.88 (d, J=4.0 Hz, 1H), 4.50-4.45 (m, 1H), 4.23-4.14 (m, 2H), 2.70-2.53 (m, 1H), 2.32-2.25 (m, 1H), 2.16-2.07 (m, 1H), 2.05-1.98 (m, 1H), 1.70-1.59 (m, 3H), 1.57-1.55 (m, 1H), 1.46 (s, 3H), 0.82 (s, 3H).

### Preparation of (E)-3-(3 fluorophenyl)prop-2-enal

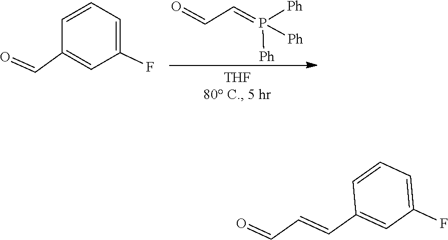 [SMILES: O=Cc1cccc(F)c1 ; O=CC=P(c1ccccc1)(c1ccccc1)c1ccccc1 ; O=C/C=C/c1cccc(F)c1]

A mixture of 3-fluorobenzaldehyde (2 g, 16.1 mmol, 1.69 mL, 1 eq) and 2-(triphenyl-5-phosphanylidene)acetaldehyde (5.39 g, 17.7 mmol, 1.1 eq) in THE (20 mL) was stirred at 80° C. for 5 hours. The mixture was concentrated under reduced pressure at 45° C. The residue was purified by flash silica gel chromatography (Biotage®; 20 g SepaFlash® Silica Flash Column, Eluent of 0-50% Ethyl acetate/Petroleum ether gradient @ 75 mL/min). The solvent was concentrated in vacuum. (E)-3-(3-fluorophenyl)prop-2-enal (1.7 g, 11.32 mmol, 70.26% yield) was obtained as yellow solid: MS m/z [M+H]⁺ (ESI): 151.1.

### Example 44. Preparation of (2S,6aS,6bR,7S,8aS,8bS,10R,11aR,12aS,12bS)-2,6b-difluoro-10-(1-fluorocyclopropyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

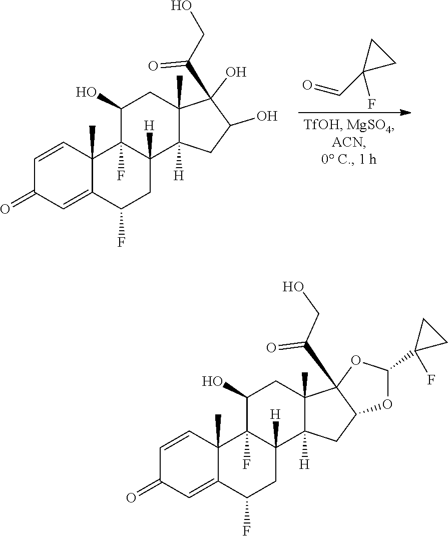

To a mixture of fluocinolone (0.15 g, 364 μmol, 1 eq) and MgSO₄ (175 mg, 1.45 mmol, 4 eq) in CH₃CN (10 mL) was added 1-fluorocyclopropanecarbaldehyde (48.1 mg, 546 μmol, 1.5 eq) and trifluoromethanesulfonic acid (164 mg, 1.09 mmol, 96.3 uL, 3 eq) at 0° C. under N₂, and then stirred at 0° C. for 1 hour. The mixture was quenched until pH=˜7 with sat.aq.NaHCO₃. The aqueous phase was extracted with ethyl acetate (20 mL×3). The combined organic phase was washed with brine (15 mL), dried with anhydrous Na₂SO₄, filtered, and concentrated in vacuum. The residue was purified by prep-HPLC (column: Phenomenex luna C₁₈ 100×40 mm×3 um; mobile phase: [water(TFA)-ACN]; B %: 5%-50%, 8 min). (2S,6aS,6bR,7S,8aS,8bS,10R,11a,12aS,12bS)-2,6b-difluoro-10-(1-fluorocyclopropyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (49.2 mg, 100 μmol, 27.56% yield, 98.29% purity) was obtained as light yellow solid: MS m/z [M+H]⁺ (ESI): 483.3; ¹H NMR (400 MHz, DMSO-d₆) δ: 7.25 (d, J=10.0 Hz, 1H), 6.29 (dd, J=1.6, 10.0 Hz, 1H), 6.11 (s, 1H), 5.75-5.46 (m, 1H), 4.86 (d, J=5.0 Hz, 1H), 4.61 (d, J=14.0 Hz, 1H), 4.43 (d, J=19.4 Hz, 1H), 4.24-4.09 (m, 2H), 2.58-2.56 (m, 1H), 2.31-2.22 (m, 1H), 2.12-2.09 (m, 1H), 2.05-1.95 (m, 1H), 1.71-1.52 (m, 3H), 1.45 (s, 3H), 1.39-1.29 (m, 1H), 1.05-1.00 (m, 2H), 0.79 (s, 3H), 0.76-0.71 (m, 2H).

### Example 45. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-phenylcyclopropyl)-1,2,6a,6b,7,8,8a,8b,11a,12,12a,12b-dodecahydro-4H-naphtho[2′,1′:4,5]indeno[1,2-d][1,3]dioxol-4-one

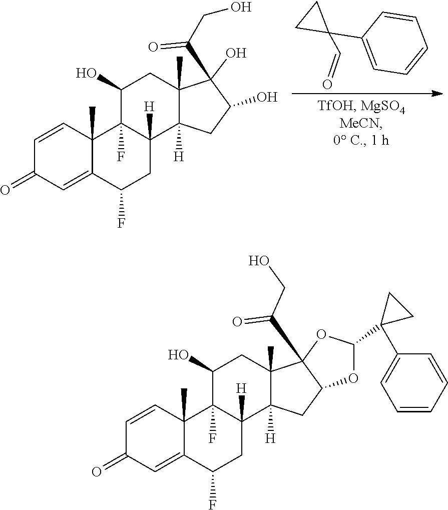

To a solution of fluocinolone (80.0 mg, 194 μmol, 1.0 eq) in MeCN (1 mL) was added MgSO₄ (93.4 mg, 776 μmol, 4.0 eq), 1-phenylcyclopropanecarbaldehyde (42.5 mg, 291 μmol, 1.5 eq) and trifluoromethanesulfonic acid (85.0 mg, 566 μmol, 0.05 mL, 2.92 eq) at 0° C. The mixture was stirred at 0° C. for 1 hour. The mixture was quenched with NaHCO₃(aq) until pH=7. The reaction mixture diluted with H₂O (15 mL) and extracted with EtOAc (10 mL×3). The combined organic layers were washed with brine 10 mL, dried over Na₂SO₄, filtered, and concentrated under reduced pressure to give a residue. The residue was purified by prep-HPLC (column: Phenomenex Luna 80×30 mm×3 um; mobile phase: [water(TFA)-ACN]; B %: 40%-60%, 8 min). (2S,6aS,6bR,7S,8aS,8bS,10R,11aR,12aS,12bS)-2,6b-difluoro-7-hydroxy-8b-(2-hydroxyacetyl)-6a,8a-dimethyl-10-(1-phenylcyclopropyl)-1,2,6a,6b,7,8,8a,8b,11a,12,12a,12b-dodecahydro-4H-naphtho[2′,1′:4,5]indeno[1,2-d][1,3]dioxol-4-one (65 mg, 119.53 μmol, 61.62% yield, 99.41% purity) was obtained as a light pink solid: MS m/z [M+H]⁺ (ESI): 541.3; ¹H NMR (400 MHz, DMSO-d₆) δ: 7.27-7.20 (m, 3H), 7.14-7.09 (m, 2H), 7.08-7.02 (m, 1H), 6.29 (dd, J=2.0, 10.0 Hz, 1H), 6.11 (s, 1H), 5.60-5.40 (m, 1H), 4.75 (d, J=4.8 Hz, 1H), 4.42-4.31 (m, 1H), 4.24 (s, 1H), 4.16-4.07 (m, 2H), 2.43-2.26 (m, 1H), 1.98-1.87 (m, 2H), 1.58 (d, J=12.8 Hz, 1H), 1.46-1.35 (m, 5H), 1.24-1.12 (m, 1H), 1.09-0.94 (m, 1H), 0.93-0.76 (m, 3H), 0.74-0.67 (m, 4H).

### Example 46. Preparation of (2S,6aS,6bR,7S,8aS,8bS,10R,11aR,12aS,12bS)-10-((E)-2-cyclobutylvinyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

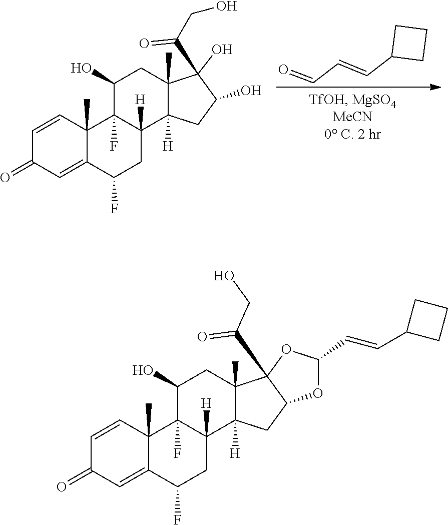

To a solution of fluocinolone (100 mg, 242 μmol, 1.0 eq) in MeCN (4 mL) was added MgSO₄ (117 mg, 970 μmol, 4.0 eq) at 0° C., (E)-3-cyclobutylprop-2-enal (40.1 mg, 364 μmol, 1.5 eq) and trifluoromethanesulfonic acid (109 mg, 727 μmol, 64.2 uL, 3.0 eq) at 0° C. The mixture was stirred at 0° C. for 2 hr under N2. The reaction mixture was quenched with aqueous of NaHCO₃ until pH to between 7 and 8. The reaction mixture was diluted by addition of H₂O (10 mL), and extracted with DCM (20 mL×3). The combined organic layers were washed with brine (10 mL), dried over Na₂SO₄, filtered, and concentrated under reduced pressure to give a residue. The residue (column: Phenomenex Luna 80×30 mm×3 um; mobile phase: [water (TFA)-ACN]; B %: 40%-65%, 8 min) was purified by prep-HPLC. (2S,6aS,6bR,7S,8aS,8bS,10R,11aR,12aS,12bS)-10-((E)-2-cyclobutylvinyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (60.4 mg, 97.6 μmol, 40.3% yield, 100% purity, TFA) was obtained as a white solid: MS m/z [M+H]⁺ (ESI): 505.3; ¹H NMR (400 MHz, DMSO-d₆) δ: 7.25 (d, J=10.4 Hz, 1H), 6.35-6.24 (m, 1H), 6.11 (s, 1H), 6.03 (dd, J=6.8, 15.6 Hz, 1H), 5.72-5.51 (m, 1H), 5.24 (dd, J=6.4, 15.6 Hz, 1H), 4.91 (d, J=6.4 Hz, 1H), 4.81 (d, J=3.2 Hz, 1H), 4.42 (d, J=19.2 Hz, 1H), 4.21-4.09 (m, 2H), 3.01-2.86 (m, 1H), 2.71-2.56 (m, 1H), 2.35-2.20 (m, 1H), 2.10-1.94 (m, 4H), 1.88-1.55 (m, 7H), 1.47 (s, 3H), 0.80 (s, 3H).

### Preparation of (E)-3-cyclobutylprop-2-enal

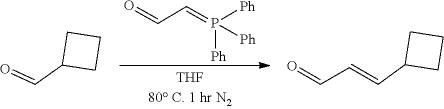

A mixture of cyclobutanecarbaldehyde (1.00 g, 11.9 mmol, 1.0 eq) and 2-(triphenyl-λ⁵-phosphanylidene)acetaldehyde (4.34 g, 14.3 mmol, 1.2 eq) in THE (20 mL) was degassed and purged with N2 for 3 times, and then stirred at 80° C. for 2 hr. The reaction mixture was concentrated under reduced pressure to give a residue. The residue was purified by column chromatography (SiO₂, Petroleum ether/Ethyl acetate=1/0 to 3/1). (E)-3-cyclobutylprop-2-enal (500 mg, 4.54 mmol, 38.2% yield) was obtained as yellow oil: ¹H NMR (400 MHz, CDCl₃) δ: 9.51 (d, J=8.0 Hz, 1H), 6.91 (dd, J=6.4, 15.6 Hz, 1H), 6.03 (dd, J=8.0, 15.6 Hz, 1H), 3.30-3.10 (m, 1H), 2.33-2.16 (m, 2H), 2.07-1.85 (m, 4H).

### Example 47. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-((R)-1-phenylethyl)-1,2,6a,6b,7,8,8a,8b,11a,12,12a,12b-dodecahydro-4H-naphtho[2′,1′:4,5]indeno[1,2-d][1,3]dioxol-4-one

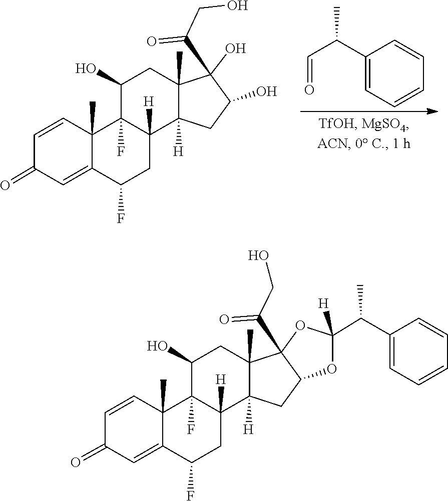

To a solution of fluocinolone (180 mg, 436 μmol, 1.0 eq) in MeCN (10 mL) was added MgSO₄ (210 mg, 1.75 mmol, 4.0 eq), (2R)-2-phenylpropanal (76.1 mg, 567 μmol, 1.3 eq) and trifluoromethanesulfonic acid (328 mg, 2.18 mmol, 193 uL, 5.0 eq) at 0° C. The mixture was stirred at 0° C. for 1 hr under N2 atmosphere. The reaction mixture was quenched until pH to ˜8 with sat. NaHCO₃, the result mixture was diluted with H₂O (10 mL), then extracted with DCM (10 mL×3). The combined organic phase was dried with anhydrous Na₂SO₄, filtered, and concentrated in vacuum. The residue was purified by prep-HPLC (column: Phenomenex Luna 80×30 mm×3 um; mobile phase: [water(TFA)-ACN]; B %: 35%-65%, 8 min) to give a crude product. The crude was further purified by SFC(column: DAICEL CHIRALPAK IC (250 mm×30 mm, 10 um); mobile phase: [0.1% NH₃H₂O ETOH]; B %: 29%-29%, 10 min) SFC (rt 1.187) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-((R)-1-phenylethyl)-1,2,6a,6b,7,8,8a,8b,11a,12,12a,12b-dodecahydro-4H-naphtho[2′,1′:4, 5]indeno[1,2-d][1,3]dioxol-4-one (42 mg, 79.5 μmol, 18.21% yield) as white solid: MS m/z [M+H]⁺ (ESI): 529.2; ¹H NMR (400 MHz, DMSO-d₆) δ: 7.31-7.07 (m, 6H), 6.29 (dd, J=2.0, 10.0 Hz, 1H), 6.17-6.10 (m, 1H), 5.66-5.46 (m, 1H), 4.84 (d, J=5.6 Hz, 1H), 4.66 (d, J=4.0 Hz, 1H), 4.37-3.99 (m, 3H), 3.01 (dq, J=4.0, 7.2 Hz, 1H), 2.60-2.52 (m, 1H), 2.23-2.10 (m, 1H), 2.08-1.89 (m, 3H), 1.77-1.45 (m, 8H), 1.43-1.28 (m, 1H), 1.27-1.22 (m, 4H), 0.81 (s, 3H).

### Preparation of (2R)-2-phenylpropanal

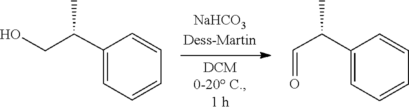

To a solution of (2R)-2-phenylpropan-1-ol (200 mg, 1.47 mmol, 1.0 eq) in DCM (5.0 mL) was added NaHCO₃ (148 mg, 1.76 mmol, 68.5 uL, 1.2 eq) and Dess-Martin (747 mg, 1.76 mmol, 546 uL, 1.2 eq) at 0° C. The mixture was stirred at 20° C. for 1 hr under N2. The reaction was quenched by addition of a 1:1 mixture of Na₂S₂O₃ (sat, aq) and NaHCO₃ (sat, aq). The aqueous phase was extracted with CH₂Cl₂ (20 mL×3) and the combined organic phases were washed with sat. NaHCO₃ (20 mL×1) and dried (Na₂SO₄), then concentrated in vacuum. The residue was purified by silica gel chromatography (column height: 250 mm, diameter: 100 mm, 100-200 mesh silica gel, Petroleum ether/Ethyl acetate=10/0, 10/1) to afford (2R)-2-phenylpropanal (140 mg, 1.04 mmol, 71.05% yield) as colorless oil: ¹H NMR (400 MHz, CDCl₃) δ: 9.70 (s, 1H), 7.44-7.29 (m, 3H), 7.25-7.19 (m, 2H), 3.72-3.57 (m, 1H), 1.46 (d, J=7.2 Hz, 3H).

### Example 48. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-((S)-1-phenylethyl)-1,2,6a,6b,7,8,8a,8b,11a,12,12a,12b-dodecahydro-4H-naphtho[2′,1′:4,5]indeno[1,2-d][1,3]dioxol-4-one

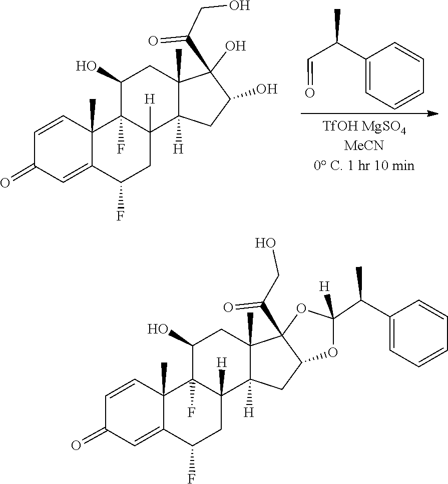

To a solution of fluocinolone (200 mg, 485 μmol, 1.0 eq), (2S)-2-phenylpropanal (97.6 mg, 727 μmol, 1.5 eq) in MeCN (4 mL) was added MgSO₄ (233 mg, 1.94 mmol, 4.0 eq) and trifluoromethanesulfonic acid (218 mg, 1.45 mmol, 128 uL, 3.0 eq) at 0° C., and then stirred at 0° C. 1 hr. The reaction mixture was quenched with aqueous of NaHCO₃ until pH to between 7 and 8. The reaction mixture was extracted with DCM (20 mL×3). The combined organic layers were washed with brine (10 mL), dried over Na₂SO₄, filtered, and concentrated under reduced pressure to give a residue. The residue was purified by prep-HPLC (column: Phenomenex Luna HILIC 250×30 mm I.D. 5 um; mobile phase: [0.1% NH₃H₂O MeOH]; B %: 20%-20%, 10 min) to give a crude product. The crude was further purified by SFC (column: Phenomenex Luna HILIC 250×30 mm I.D. 5 um; mobile phase: [0.1% NH₃H₂O MeOH]]; B %: 20%-20%, 10 min). (2S,6aS,6bR,7S,8aS,8bS,10R,11aR,12aS,12bS)-2,6b-difluoro-7-hydroxy-8b-(2-hydroxyacetyl)-6a,8a-dimethyl-10-((S)-1-phenylethyl)-1,2,6a,6b,7,8,7,8,8a,8b,11a,12,12a,12b-dodecahydro-4H-naphtho[2′,1′:4,5]indeno[1,2-d][1,3]dioxol-4-one (19.3 mg, 36.5 μmol, 7.53% yield) was obtained as a white solid: MS m/z [M+H]⁺ (ESI): 529.2; ¹H NMR (400 MHz, DMSO-d₆) δ: 7.31 7.06 (m, 6H), 6.31-6.22 (m, 1H), 6.19-6.08 (m, 1H), 5.66-5.43 (m, 1H), 4.84-4.76 (m, 1H), 4.67-4.60 (m, 1H), 4.41-3.90 (m, 3H), 3.01-2.95 (m, 1H), 2.48-2.13 (m, 2H), 2.08-1.73 (m, 2H), 1.70-1.48 (m, 6H), 1.43-1.27 (m, 1H), 1.26-1.18 (m, 3H), 1.17-1.01 (m, 1H), 0.80 (s, 3H).

### Preparation of (2S)-2-phenylpropanal

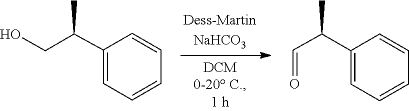

A mixture of (2S)-2-phenylpropan-1-ol (250 mg, 1.84 mmol, 1.0 eq), NaHCO₃ (185 mg, 2.20 mmol, 85.7 uL, 1.2 eq), and Dess-Martin (934 mg, 2.20 mmol, 682 uL, 1.2 eq) in DCM (5 mL) was degassed and purged with N2 for 3 times, and then stirred at 0° C. under N2 atmosphere for 1 hr. The reaction mixture was quenched by addition of aq Na₂S₂O₃ (10 mL), and extracted with DCM (20 mL×3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=1/0 to 5/1). (2S)-2-phenylpropanal (0.200 g, 1.49 mmol, 81.2% yield) was obtained as yellow oil: ¹H NMR (400 MHz, CDCl₃) δ: 9.67 (d, J=1.2 Hz, 1H), 7.20 (d, J=7.2 Hz, 2H), 7.40-7.17 (m, 3H), 3.62 (q, J=7.2 Hz, 1H), 1.43 (d, J=7.2 Hz, 3H).

### Example 49. Preparation of (6aS,6bR,7S,8aS,8bS,10R,11aR,12aS,12bS)-10-cyclobutyl-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

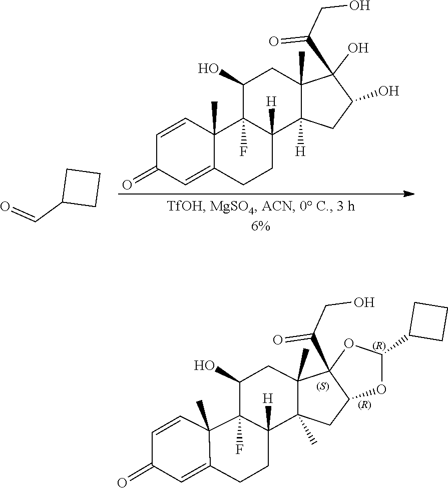

To a stirred solution of cyclobutyral (500 mg, 5.94 mmol, 1.5 equiv) and triamcinolone (1.5 g, 3.96 mmol, 1.0 equiv) in acetonitrile was added trifluoromethanesulfonic acid (1.7 g, 11.89 mmol, 3.0 equiv) dropwise at 0° C. under nitrogen atmosphere. The mixture was basified to pH 7 with saturated sodium bicarbonate (aq.). The aqueous layer was extracted with methylene chloride (3×30 mL). The crude product was purified by Prep-HPLC with the following conditions (column, C₁₈ silica gel; mobile phase, acetonitrile in Water (0.1% ammonia+10 mmol/L ammonium bicarbonate) to afford (6aS,6bR,7S,8aS,8bS,10R,11aR,12aS,12bS)-10-cyclobutyl-6b-fluoro-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (110.2 mg, 6%) as a white solid: MS m/z [M+H]⁺ (ESI): 461.15; ¹H NMR (300 MHz, Methanol-d₄) δ: 0.94 (s, 3H), 1.40-1.50 (m, 1H), 1.58 (s, 3H), 1.60-1.68 (m, 3H), 1.94-1.99 (m, 7H), 2.10-2.30 (m, 2H), 2.41-2.70 (m, 4H), 3.31-3.32 (m, 1H), 4.26-4.32 (m, 2H), 4.50-4.57 (m, 1H), 6.09 (s, 1H), 6.30-6.40 (m, 1H), 7.39 (d, J=10.2 Hz, 1H).

### Example 50. Preparation of (6aS,6bR,7S,8aS,8bS,10R,11aR,12aS,12bS)-6b-fluoro-10-(3-fluorothiophen-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

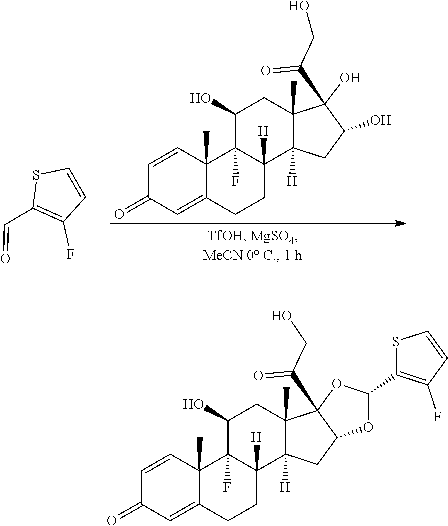

To a solution of triamcinolone (500 mg, 1.27 mmol, 1.0 eq) in MeCN (5 mL) was added MgSO₄ (610 mg, 5.07 mmol, 4 eq), 3-fluorothiophene-2-carbaldehyde (245 mg, 1.88 mmol, 1.49 eq) and trifluoromethanesulfonic acid (578 mg, 3.85 mmol, 0.34 mL, 3.04 eq) at 0° C. The result mixture was stirred at 0° C. for 1 hour. The mixture was quenched with NaHCO₃(aq) until pH=7. The reaction mixture was diluted with H₂O (15 mL) and extracted with EtOAc (10 mL×3). The combined organic layers were washed with brine (10 mL), dried over Na₂SO₄, filtered, and concentrated under reduced pressure to give a residue. The residue was purified by prep-HPLC (column: Phenomenex Luna C₁₈ 250 50 mm 10 um; mobile phase: [water(TFA)-ACN]; B %: 30%-50%, 10 min). (6aS,6bR,7S,8aS,8bS,10R,11aR,12aS,12bS)-6b-fluoro-10-(3-fluorothiophen-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (255 mg, 503.40 μmol, 39.71% yield) was obtained as a white solid: MS m/z [M+H]⁺ (ESI): 507.3; ¹H NMR (400 MHz, DMSO-d₆) δ: 7.57-7.55 (m, 1H), 7.28 (d, J=10.0 Hz, 1H), 6.93 (d, J=5.6 Hz, 1H), 6.23 (d, J=9.6 Hz, 1H), 6.03 (s, 1H), 5.88 (s, 1H), 4.91 (d, J=4.0 Hz, 1H), 4.47 (d, J=19.6 Hz, 1H), 4.22-4.12 (m, 2H), 2.68-2.57 (m, 1H), 2.36-2.33 (m, 1H), 2.19-2.07 (m, 1H), 2.02-1.99 (m, 1H), 1.89-1.80 (m, 1H), 1.69-1.56 (m, 3H), 1.47 (s, 3H), 1.40-1.34 (m, 1H), 0.84 (s, 3H).

### Preparation of (3 fluoro-2-thienyl)methanol

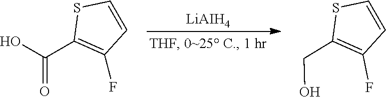

To a solution of 3-fluorothiophene-2-carboxylic acid (1.00 g, 6.84 mmol, 1.0 eq) in THE (10 mL) was added LiAlH₄ (363 mg, 9.58 mmol, 1.4 eq) at 0° C. The resulting mixture was stirred at 25° C. for 1 hour. The reaction mixture was quenched by addition sat. Na₂SO₄ (aq) 1 mL at 0° C., and then dried over Na₂SO₄, filtered, and concentrated under reduced pressure to give a residue. (3-fluoro-2-thienyl)methanol (1 g, crude) was obtained as a white solid: ¹H NMR (400 MHz, DMSO-d₆) δ: 7.44 (dd, J=4.0, 5.2 Hz, 1H), 6.92 (d, J=5.6 Hz, 1H), 5.44 (t, J=5.6 Hz, 1H), 4.54 (d, J=6.0 Hz, 2H).

### Preparation of 3 fluorothiophene-2-carbaldehyde

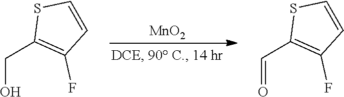

To a solution of (3-fluoro-2-thienyl)methanol (900 mg, 6.81 mmol, 1 eq) in DCE (10 mL) was added MnO₂ (2.96 g, 34.1 mmol, 5.0 eq). The mixture was stirred at 90° C. for 14 hours. The reaction mixture was filtered and concentrated under reduced pressure to give a residue. The residue was purified by flash silica gel chromatography (Biotage®; 20 g SepaFlash® Silica Flash Column, Eluent of 10-30% Ethyl acetate/petroleum ether gradient @ 60 mL/min). 3-Fluorothiophene-2-carbaldehyde (0.5 g, 3.84 mmol, 56.42% yield) was obtained as a yellow solid: MS m/z [M+H]⁺ (ESI): 131.0; ¹H NMR (400 MHz, CDCl₃) δ: 10.04 (s, 1H), 7.66 (t, J=2.8 Hz, 1H), 6.91 (d, J=5.6 Hz, 1H).

### Example 51. Preparation of (6aR,6bS,7S,8aS,8bS,10R,11aR,12aS,12bS)-10-(3-fluorothiophen-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

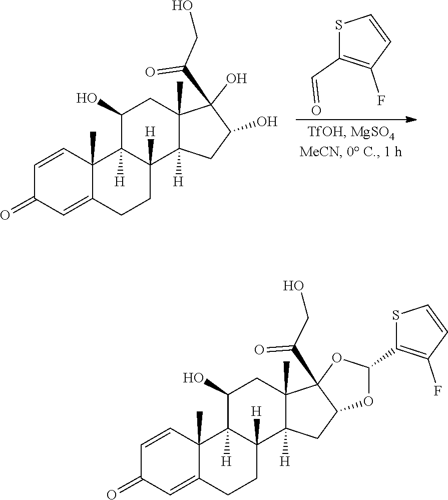

To a solution of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (500 mg, 1.33 mmol, 1.0 eq) in MeCN (5 mL) was added MgSO₄ (640 mg, 5.31 mmol, 4.0 eq), 3-fluorothiophene-2-carbaldehyde (242 mg, 1.86 mmol, 1.4 eq) and trifluoromethanesulfonic acid (612 mg, 4.08 mmol, 360 uL, 3.07 eq) at 0° C. The mixture was stirred at 0° C. for 1 hour. The result mixture was stirred at 0° C. for 1 hour. The mixture was quenched with NaHCO₃(aq) until pH=7. The reaction mixture was diluted with H₂O (30 mL) at 0° C. and extracted with EtOAc (30 mL×3). The combined organic layers were washed with brine (30 mL), dried over Na₂SO₄, filtered, and concentrated under reduced pressure to give a residue. The residue was purified by prep-HPLC (column: Phenomenex Luna C₁₈ 250×50 mm×10 um; mobile phase: [water(TFA)-ACN]; B %: 30%-50%, 10 min). (6aR,6b S,7S,8aS,8b S,10R,11aR,12aS,12b S)-10-(3-fluorothiophen-2-yl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (355 mg, 726.61 μmol, 54.71% yield) was obtained as a white solid: MS m/z [M+H]⁺ (ESI): 489.3; ¹H NMR (400 MHz, DMSO-d₆) δ: 7.54 7.51 (m, 1H), 7.32 (d, J=10.0 Hz, 1H), 6.92 (d, J=5.6 Hz, 1H), 6.17 (d, J=10.0 Hz, 1H), 5.93 (s, 1H), 5.85 (s, 1H), 4.90 (d, J=3.2 Hz, 1H), 4.46 (d, J=19.6 Hz, 1H), 4.29 (s, 1H), 4.15 (d, J=19.6 Hz, 1H), 2.53-2.49 (m, 1H), 2.31-2.29 (m, 1H), 2.17-2.00 (m, 2H), 1.77-1.57 (m, 5H), 1.36 (s, 3H), 1.01-0.91 (m, 1H), 0.82 (s, 3H).

### Example 52. Preparation of (2S,6aS,6bR,7S,8aS,8bS,10R,11aR,12aS,12bS)-2,6b-difluoro-7-hydroxy-8b-(2-hydroxyacetyl)-6a,8a-dimethyl-10-(thieno[3,2-b]thiophen-2-yl)-1,2,6a,6b,7,8,8a,8b,11a,12,12a,12b-dodecahydro-4H-naphtho[2′,1′:4,5]indeno[1,2-d][1,3]dioxol-4-one

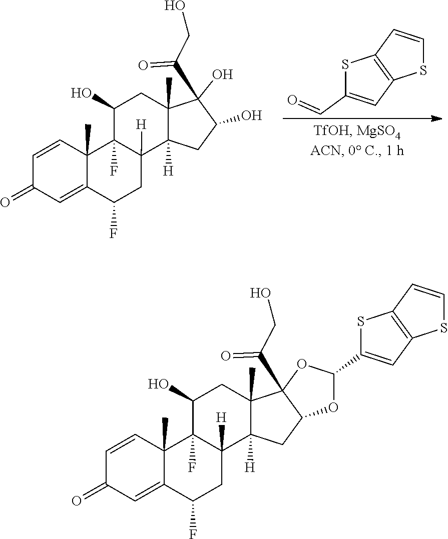

To a solution of fluocinolone (100 mg, 242 μmol, 1.0 eq) in MeCN (1 mL) was added MgSO₄ (146 mg, 1.21 mmol, 5.0 eq), thieno[3,2-b]thiophene-5-carbaldehyde (61.2 mg, 364 μmol, 1.5 eq) and trifluoromethanesulfonic acid (TfOH, 146 mg, 970 μmol, 85.6 uL, 4.0 eq) at 0° C. The mixture was stirred at 0° C. for 1 hr. The pH of mixture was adjusted to 8 with sat.NaHCO₃, the result mixture was diluted with water (10 mL), then was extracted with DCM (10 mL×3). The combined organic phase was dried with anhydrous Na₂SO₄, filtered, and concentrated in vacuum. The residue was purified by prep-HPLC(column: Phenomenex Luna 80×30 mm×3 um; mobile phase: [water(TFA)-ACN]; B %: 35%-60%, 8 min) to afford (2S,6aS,6bR,7S,8aS,8bS,10R,11aR,12aS,12bS)-2,6b-difluoro-7-hydroxy-8b-(2-hydroxyacetyl)-6a,8a-dimethyl-10-(thieno[3,2-b]thiophen-2-yl)-1,2,6a,6b,7,8,8a,8b,11a,12,12a,12b-dodecahydro-4H-naphtho[2′,1′:4,5]indeno[1,2-d][1,3]dioxol-4-one (70 mg, 124 μmol, 51.31% yield) as white solid: MS m/z [M+H]⁺ (ESI): 563.1; ¹H NMR (400 MHz, DMSO-d₆) δ: 7.69 (d, J=5.4 Hz, 1H), 7.62 (s, 1H), 7.46 (d, J=5.4 Hz, 1H), 7.30-7.24 (m, 1H), 6.30 (dd, J=2.0, 10.0 Hz, 1H), 6.14 (s, 1H), 5.93 (s, 1H), 5.76-5.56 (m, 1H), 4.97 (t, J=2.4 Hz, 1H), 4.55 (d, J=19.6 Hz, 1H), 4.25-4.16 (m, 2H), 2.67-2.57 (m, 1H), 2.32-2.29 (m, 1H), 2.08-2.00 (m, 1H), 1.74-1.65 (m, 3H), 1.58-1.47 (m, 4H), 0.86 (s, 3H).

### Example 53. Preparation of (2S,6aS,6bR,7S,8aS,8bS,10R,11aR,12aS,12bS)-10-(3-aminophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

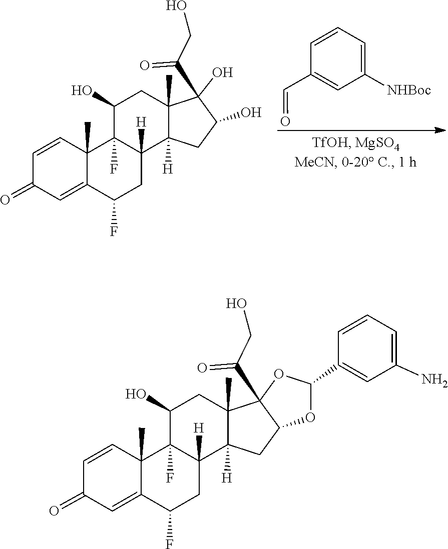

To a solution of fluocinolone (100 mg, 242 μmol, 1 eq) in MeCN (2 mL) was added MgSO₄ (117 mg, 970 μmol, 4 eq), tert-butyl N-(3-formylphenyl)carbamate (107 mg, 485 μmol, 2 eq) and TfOH (364 mg, 2.42 mmol, 214 uL, 10 eq) at 0° C., and then stirred at 20° C. for 1 hour. The reaction mixture was quenched with sat. NaHCO₃ until pH=˜8, diluted with water (10 mL), then extracted with DCM (10 mL×3). The combined organic phase was dried with anhydrous Na₂SO₄, filtered, and concentrated in vacuum. The residue was purified by prep-HPLC(column: Phenomenex Luna 80×30 mm×3 um; mobile phase: [water(TFA)-ACN]; B %: 20%-40%, 8 min) to afford (2S,6aS,6bR,7S,8aS,8bS,10R,11aR,12aS,12bS)-10-(3-aminophenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (64 mg, 102 μmol, 41.9% yield, TFA) as white solid: MS m/z [M+H]⁺ (ESI): 516.3; ¹H NMR (400 MHz, DMSO-d₆) δ: 7.30-7.21 (m, 2H), 7.02-6.88 (m, 3H), 6.29 (dd, J=1.6, 10.4 Hz, 1H), 6.12 (s, 1H), 5.74-5.54 (m, 1H), 5.39 (s, 1H), 4.95 (d, J=3.2 Hz, 1H), 4.50 (d, J=19.2 Hz, 1H), 4.19 (d, J=19.6 Hz, 2H), 2.55-2.50 (m, 1H), 2.29-2.27 (m, 1H), 2.24-2.10 (m, 1H), 2.06-1.95 (m, 1H), 1.76-1.66 (m, 3H), 1.50-1.45 (m, 4H), 0.85 (s, 3H).

### Example 54. Preparation of (6aR,6bS,7S,8aS,8bS,10R,11aR,12aS,12bS)-10-((E)-2-(3-fluorothiophen-2-yl)vinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

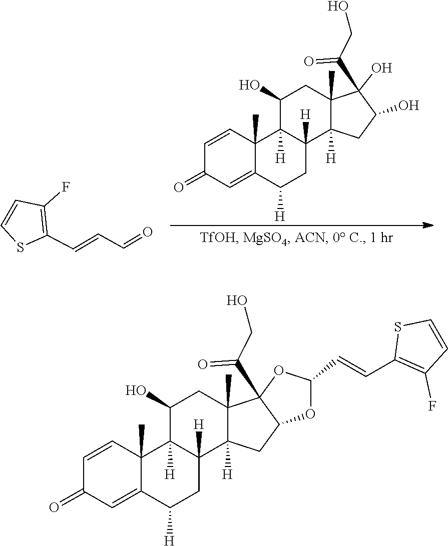

To a mixture of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (0.2 g, 531 μmol, 1 eq) and MgSO₄ (255 mg, 2.13 mmol, 4 eq) in CH₃CN (5 mL) was added (E)-3-(3-fluoro-2-thienyl)prop-2-enal (124 mg, 796 μmol, 1.5 eq) and TfOH (119 mg, 796 μmol, 70.3 uL, 1.5 eq) at 0° C. under N2, and then stirred at 0° C. for 1 hour. The mixture was quenched until pH=˜7 with sat. aq. NaHCO₃. The aqueous phase was extracted with ethyl acetate (40 mL×3). The combined organic phase was washed with brine (30 mL), dried with anhydrous Na₂SO₄, filtered, and concentrated in vacuum. The residue was purified by prep-HPLC (column: Phenomenex Luna C₁₈ 100×40 mm×3 um; mobile phase: [water(TFA)-ACN]; B %: 15%-60%, 8 min). (6aR,6bS,7S,8aS,8bS,10R,11aR,12aS,12bS)-10-((E)-2-(3-fluorothiophen-2-yl)vinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (84 mg, 160.87 μmol, 30.28% yield, 98.55% purity) was obtained as a white solid: MS m/z [M+H]⁺ (ESI): 515.3; ¹H NMR (400 MHz, DMSO-d₆) δ: 7.46 (t, J=4.8 Hz, 1H), 7.32 (d, J=10.0 Hz, 1H), 6.95 (d, J=5.2 Hz, 1H), 6.88 (d, J=16.0 Hz, 1H), 6.16 (dd, J=2.0, 10.0 Hz, 1H), 5.92 (s, 1H), 5.81 (dd, J=6.4, 16.0 Hz, 1H), 5.05 (d, J=6.4 Hz, 1H), 4.81 (d, J=4.4 Hz, 1H), 4.45 (d, J=19.2 Hz, 1H), 4.28 (d, J=2.8 Hz, 1H), 4.14 (d, J=19.2 Hz, 1H), 2.35-2.23 (m, 1H), 2.10-1.94 (m, 2H), 1.78-1.49 (m, 6H), 1.35 (s, 3H), 1.03-0.89 (m, 2H), 0.79 (s, 3H).

### Example 55. Preparation of (6aR,6bS,7S,8aS,8bS,10R,11aR,12aS,12bS)-10-((E)-2-cyclopropylvinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

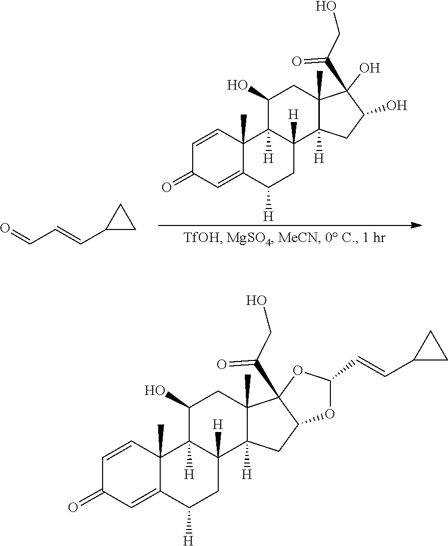

To a mixture of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (120 mg, 319 μmol, 1.0 eq) and MgSO₄ (153 mg, 1.28 mmol, 4.0 eq) in MeCN (10 mL) was added (Z)-3-cyclopropylprop-2-enal (46.0 mg, 478 μmol, 1.5 eq) and TfOH (47.8 mg, 319 μmol, 28.14 uL, 1.0 eq) at 0° C. under N2, and then stirred at 0° C. for 1 hour. The mixture was quenched with NaHCO₃(aq) until pH=˜7. The aqueous phase was extracted with ethyl acetate (20 mL×3). The combined organic phase was washed with brine (15 mL), dried with anhydrous Na₂SO₄, filtered, and concentrated in vacuum. The residue was purified by prep-HPLC (column: Phenomenex Luna 80×30 mm×3 um; mobile phase: [water(TFA)-ACN]; B %: 40%-60%, 8 min). (6aR,6bS,7S,8aS,8bS,10R,11aR,12aS,12bS)-10-((E)-2-(3-fluorothiophen-2-yl)vinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (99.4 mg, 218.68 μmol, 68.60% yield) was obtained as a white solid: MS m/z [M+H]⁺ (ESI): 455.3; ¹H NMR (400 MHz, DMSO-d₆) δ: 7.32 (d, J=10.0 Hz, 1H), 6.17 (dd, J=2.0, 10.0 Hz, 1H), 5.93 (s, 1H), 5.52-5.38 (m, 2H), 4.82 (d, J=6.0 Hz, 1H), 4.74 (d, J=4.0 Hz, 1H), 4.38 (d, J=19.6 Hz, 1H), 4.28 (d, J=2.8 Hz, 1H), 4.11 (d, J=19.6 Hz, 1H), 2.34-2.26 (m, 1H), 2.11-1.94 (m, 2H), 1.77-1.64 (m, 2H), 1.60-1.48 (m, 3H), 1.44-1.38 (m, 1H), 1.36 (s, 3H), 1.06-0.97 (m, 1H), 0.96-0.91 (m, 1H), 0.78 (m, 3H), 0.74-0.66 (m, 2H), 0.39-0.31 (m, 2H).

### Preparation of (Z)-3-cyclopropylprop-2-enal

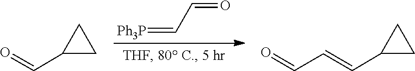

To a solution of cyclopropanecarbaldehyde (3.00 g, 42.8 mmol, 3.20 mL, 1 eq) in THF (30 mL) was added 2-(triphenyl-5-phosphanylidene)acetaldehyde (14.3 g, 47.1 mmol, 1.1 eq), and then stirred at 80° C. for 5 hours. The reaction mixture was concentrated under reduced pressure to give a residue. The residue was purified by flash silica gel chromatography (Biotage®; 20 g SepaFlash® Silica Flash Column, Eluent of 10-20% Ethyl acetate/petroleum ether gradient @ 60 mL/min). The eluent was removed under reduced pressure. (Z)-3-Cyclopropylprop-2-enal (500 mg, 5.20 mmol, 12.15% yield) was obtained as a yellow oil: ¹H NMR (400 MHz, CDCl₃) δ: 9.40 (d, J=7.6 Hz, 1H), 6.32-6.14 (m, 2H), 1.76-1.65 (m, 1H), 1.12-1.05 (m, 2H), 0.77-0.71 (m, 2H).

### Preparation of (E)-3-cyclobutylprop-2-enal

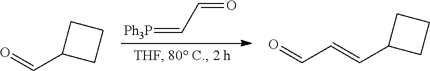

A mixture of cyclobutanecarbaldehyde (1 g, 11.9 mmol, 1 eq) and 2-(triphenyl-5-phosphanylidene)acetaldehyde (4.34 g, 14.3 mmol, 1.2 eq) in THF (20 mL) was degassed and purged with N2 for 3 times, and then stirred at 80° C. for 2 hours under N2 atmosphere. The reaction mixture was concentrated under reduced pressure to give a residue. The residue was purified by flash silica gel chromatography (ISCO®; 12 g SepaFlash® Silica Flash Column, Eluent of 0-20% Ethyl acetate/Petroleum ether gradient @ 50 mL/min) to give (E)-3-cyclobutylprop-2-enal (230 mg, 2.09 mmol, 17.6% yield) as green oil: ¹H NMR (400 MHz, CDCl₃) δ: 9.45 (d, J=7.6 Hz, 1H), 6.93-6.79 (m, 1H), 6.02-6.53 (m, 1H), 3.24-3.10 (m, 1H), 2.27-2.06 (m, 2H), 2.02-1.86 (m, 4H).

### Example 56. Preparation of (6aR,6bS,7S,8aS,8bS,10R,11aR,12aS,12bS)-10-((E)-2-cyclobutylvinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

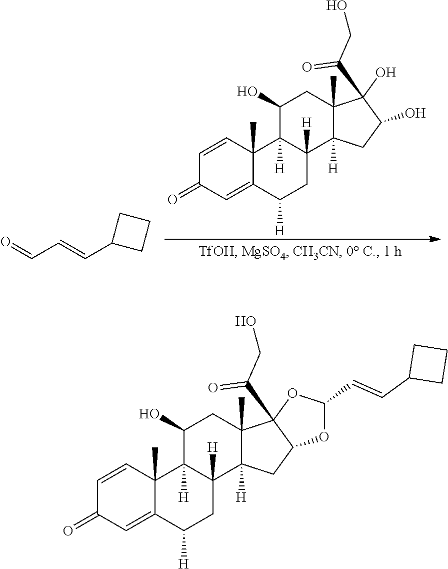

To a solution of (E)-3-cyclobutylprop-2-enal (351 mg, 3.19 mmol, 1.2 eq) in CH₃CN (15 mL) was added MgSO₄ (1.60 g, 13.3 mmol, 5 eq), (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (1 g, 2.66 mmol, 1 eq) and TfOH (1.02 g, 6.80 mmol, 600 uL, 2.56 eq) at 0° C. The mixture was stirred at 0° C. for 1 hour. The reaction mixture was quenched with NaHCO₃(aq) until pH=˜7 at 0° C., and then diluted with H₂O (30 mL) and extracted with DCM (15 mL×3). The combined organic layers were washed with brine (15 mL), dried over Na₂SO₄, filtered, and concentrated under reduced pressure to give a residue. The residue was purified by prep-HPLC (TFA condition, column: Phenomenex Luna C₁₈ (250×70 mm, 15 um); mobile phase: [water(TFA)-ACN]; B %: 43%-63%, 27 min) to give (6aR,6bS,7S,8aS,8bS,10R,11a,12aS,12bS)-10-((E)-2-cyclobutylvinyl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (1.04 g, 2.22 mmol, 83.5% yield) was obtained as a white solid: MS m/z [M+H]⁺ (ESI): 469.1; ¹H NMR (400 MHz, DMSO-d₆) δ: 7.31 (d, J=10.4 Hz, 1H), 6.19-6.13 (m, 1H), 6.04-5.96 (m, 1H), 5.92 (s, 1H), 5.36-5.26 (m, 1H), 4.86 (d, J=6.8 Hz, 1H), 4.77 (d, J=4.0 Hz, 1H), 4.45-4.36 (m, 1H), 4.28 (d, J=2.8 Hz, 1H), 4.17-4.08 (m, 1H), 2.99-2.85 (m, 1H), 2.54-2.50 (m, 1H), 2.36-2.23 (m, 1H), 2.15-1.91 (m, 4H), 1.89-1.65 (m, 6H), 1.64-1.46 (m, 3H), 1.37 (s, 3H), 1.08-0.92 (m, 2H), 0.80 (s, 3H).

### Example 57. Preparation of (6aS,6bR,7S,8aS,8bS,10R,11aR,12aS,12bS)-10-((E)-2-cyclobutylvinyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

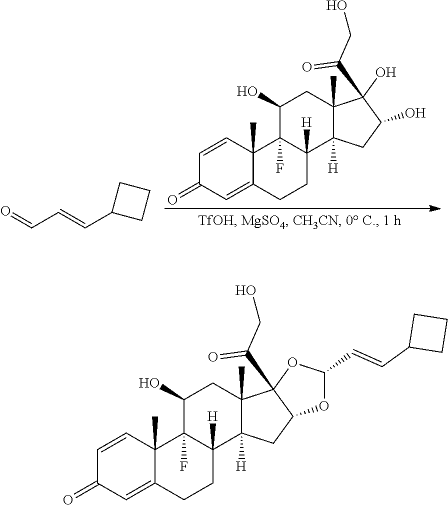

To a solution of (E)-3-cyclobutylprop-2-enal (335 mg, 3.04 mmol, 1.2 eq) in CH₃CN (15 mL) was added MgSO₄ (1.53 g, 12.7 mmol, 5 eq), triamcinolone (1 g, 2.54 mmol, 1 eq) and TfOH (681 mg, 4.54 mmol, 401 uL, 1.79 eq) at 0° C. The mixture was stirred at 0° C. for 1 hour.

The reaction mixture was quenched with NaHCO₃(aq) until pH=˜7 at 0° C., and then diluted with H₂O (30 mL) and extracted with DCM (15 mL×3). The combined organic layers were washed with brine (15 mL), dried over Na₂SO₄, filtered, and concentrated under reduced pressure to give a residue. The residue was purified by prep-HPLC (TFA condition, column: Phenomenex Luna C₁₈ (250×70 mm, 15 um); mobile phase: [water(TFA)-ACN]; B %: 43%-63%, 27 min) to give (6aS,6bR,7S,8aS,8bS,10R,11aR,12aS,12bS)-10-((E)-2-cyclobutylvinyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (656 mg, 1.35 mmol, 53.2% yield) as a white solid: MS m/z [M+H]⁺ (ESI): 487.2; ¹H NMR (400 MHz, DMSO-d₆) δ: 7.28 (d, J=10.4 Hz, 1H), 6.26-6.19 (m, 1H), 6.08-5.97 (m, 2H), 5.26-5.18 (m, 1H), 4.90 (d, J=6.4 Hz, 1H), 4.79 (d, J=4.0 Hz, 1H), 4.46-4.37 (m, 1H), 4.20-4.09 (m, 2H), 3.00-2.87 (m, 1H), 2.70-2.56 (m, 1H), 2.44-2.26 (m, 2H), 2.08-1.89 (m, 4H), 1.88-1.67 (m, 5H), 1.63 (d, J=13.6 Hz, 1H), 1.58-1.51 (m, 2H), 1.47 (s, 3H), 1.40-1.27 (m, 1H), 0.81 (s, 3H).

### Example 58. Preparation of (2S,6aS,6bR,7S,8aS,8bS,10R,11aR,12aS,12bS)-10-(5-(3-aminobenzyl)thiophen-2-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

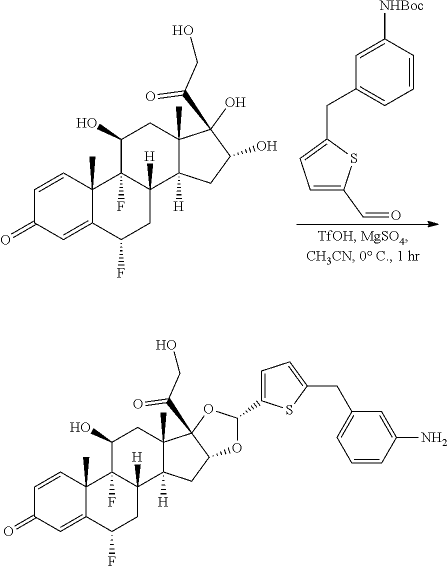

To a mixture of fluocinolone (0.15 g, 363 μmol, 1 eq) and MgSO₄ (175 mg, 1.45 mmol, 4 eq) in CH₃CN (5 mL) was added tert-butyl N-[3-[(5-formyl-2-thienyl)methyl]phenyl]carbamate (115 mg, 363 μmol, 1 eq) and TfOH (163 mg, 1.09 mmol, 96.3 uL, 3 eq) at 0° C. under N2, and then stirred at 0° C. for 1 hour. The mixture was quenched until pH=˜7 with sat. aq.NaHCO₃. The aqueous phase was extracted with ethyl acetate (30 mL×3). The combined organic phase was washed with brine (20 mL), dried with anhydrous Na₂SO₄, filtered, and concentrated in vacuum. The residue was purified by prep-HPLC (column: Phenomenex Luna C₁₈ 75×30 mm×3 um; mobile phase: [water(TFA)-ACN]; B %: 5%-50%, 8 min). (2S,6aS,6bR,7S,8aS,8bS,10R,11aR,12aS,12bS)-10-(5-(3-aminobenzyl)thiophen-2-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (57.7 mg, 89.83 μmol, 24.70% yield, 95.22% purity) was obtained as a light yellow solid: MS m/z [M+H]⁺ (ESI): 612.4; ¹H NMR (400 MHz, DMSO-d₆) δ: 7.25 (t, J=8.8 Hz, 1H), 7.08-7.02 (m, 2H), 7.01-6.92 (m, 2H), 6.75 (d, J=3.6 Hz, 1H), 6.31 (dd, J=1.6, 10.0 Hz, 1H), 6.16 (s, 1H), 5.72 (s, 1H), 5.68-5.50 (m, 1H), 4.87 (d, J=5.2 Hz, 1H), 4.46 (d, J=19.4 Hz, 1H), 4.20-4.11 (m, 2H), 4.07 (s, 2H), 2.70-2.55 (m, 1H), 2.32-2.29 (m, 1H), 2.18-2.10 (m, 1H), 1.96-1.93 (m, 1H), 1.66-1.58 (m, 2H), 1.42-138 (m, 4H), 0.80 (s, 3H).

### Preparation of tert-butyl N-[3-[(5 formyl-2-thienyl)methyl]phenyl]carbamate

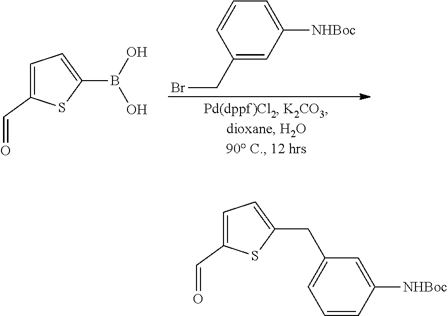 [SMILES: O=Cc1ccc(B(O)O)s1 ; CC(C)(C)OC(=O)Nc1cccc(CBr)c1 ; CC(C)(C)OC(=O)Nc1cccc(Cc2ccc(C=O)s2)c1]

To a mixture of (5-formyl-2-thienyl)boronic acid (0.1 g, 641 μmol, 1 eq) and tert-butyl N-[3-(bromomethyl)phenyl]carbamate (201 mg, 705 μmol, 1.1 eq) in dioxane (5 mL) and H₂O (1 mL) was added K₂CO₃ (177 mg, 1.28 mmol, 2 eq) and Pd(dppf)Cl₂ (46.9 mg, 64.1 μmol, 0.1 eq) at 25° C. under N2, and then stirred at 90° C. for 12 hours. The reaction mixture was filtered, and the filtrate was concentrated. The residue was purified by flash silica gel chromatography (Biotage®; 4 g SepaFlash® Silica Flash Column, Eluent of 0-50% Ethyl acetate/Petroleum ether gradient @ 40 mL/min). tert-Butyl N-[3-[(5-formyl-2-thienyl)methyl]phenyl]carbamate (0.13 g, 409 μmol, 63.88% yield) was obtained as a yellow solid: ¹H NMR (400 MHz, DMSO-d₆) δ: 9.81 (s, 1H), 7.87 (d, J=3.6 Hz, 1H), 7.44 (s, 1H), 7.27 (s, 1H), 7.20 (t, J=8.0 Hz, 1H), 7.11 (d, J=4.0 Hz, 1H), 6.89 (d, J=7.6 Hz, 1H), 4.18 (s, 2H), 1.46 (s, 9H).

### Example 59. Preparation of (2S,6aS,6bR,7S,8aS,8bS,10R,11aR,12aS,12bS)-10-(4-(3-aminobenzyl)thiophen-2-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

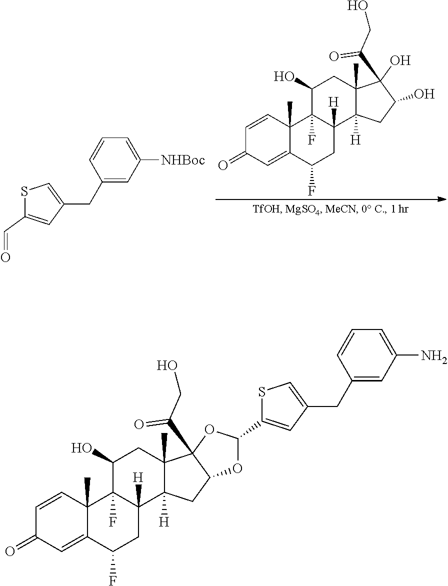

To a mixture of fluocinolone (100 mg, 242 μmol, 1.0 eq) and MgSO₄ (117 mg, 970 μmol, 4.0 eq) in MeCN (1 mL) was added tert-butyl N-[3-[(5-formyl-3-thienyl)methyl]phenyl]carbamate (100 mg, 315 μmol, 1.30 eq) and TfOH (109 mg, 727 μmol, 64.2 uL, 3.0 eq) at 0° C. under N2, and then stirred at 0° C. for 1 hour. The mixture was quenched with NaHCO₃(aq) until pH=˜7. The reaction mixture was filtered and concentrated under reduced pressure to give a residue. The residue was purified by prep-HPLC (column: Phenomenex Luna 80×30 mm×3 um; mobile phase: [water(TFA)-ACN]; B %: 25%-45%, 8 min). (2S,6aS,6bR,7S,8aS,8bS,10R,11aR,12aS,12bS)-10-(4-(3-aminobenzyl)thiophen-2-yl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (97 mg, 154.36 μmol, 63.66% yield, 97.34% purity) was obtained as a white solid: MS m/z [M+H]⁺ (ESI): 612.4; ¹H NMR (400 MHz, DMSO-d₆) δ: 7.31 (t, J=7.6 Hz, 1H), 7.26 (d, J=10.0 Hz, 2H), 7.11 (d, J=7.6 Hz, 1H), 7.05 (d, J=1.2 Hz, 1H), 7.03-6.97 (m, 2H), 6.30 (dd, J=2.0, 10.4 Hz, 1H), 6.13 (s, 1H), 5.79 (s, 1H), 5.73-5.54 (m, 1H), 4.90 (d, J=4.4 Hz, 1H), 4.46 (d, J=19.6 Hz, 1H), 4.19-4.13 (m, 2H), 3.86 (s, 2H), 2.71-2.61 (m, 1H), 2.35-2.27 (m, 1H), 2.27-2.19 (m, 1H), 2.03-1.95 (m, 1H), 1.69-1.61 (m, 3H), 1.48 (s, 3H), 1.46-1.41 (m, 1H), 0.83 (s, 3H).

### Preparation of tert-butyl N-[3-[(5 formyl-3-thienyl)methyl]phenyl]carbamate

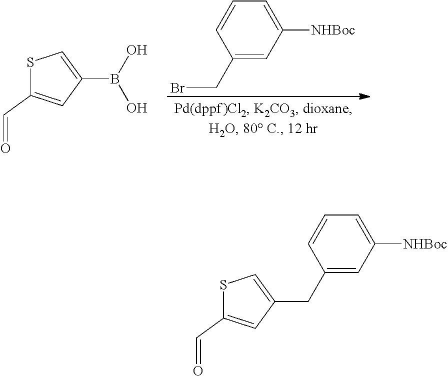 [SMILES: O=Cc1cc(B(O)O)cs1 ; CC(C)(C)OC(=O)Nc1cccc(CBr)c1 ; CC(C)(C)OC(=O)Nc1cccc(Cc2csc(C=O)c2)c1]

To a mixture of (5-formyl-3-thienyl)boronic acid (0.10 g, 641 μmol, 1.01 eq) and tert-butyl N-[3-(bromomethyl)phenyl]carbamate (200 mg, 699 μmol, 1.1 eq) and K₂CO₃ (176 mg, 1.27 mmol, 2.0 eq) in dioxane (2 mL) and H₂O (0.1 mL) was added Pd(dppf)Cl₂ (46.5 mg, 63.5 μmol, 0.1 eq) in one portion at 25° C. under N2, and then stirred at 80° C. for 12 hours. The reaction mixture was filtered and concentrated under reduced pressure to give a residue. The residue was purified by flash silica gel chromatography (Biotage®; 20 g SepaFlash® Silica Flash Column, Eluent of 20-35% Ethyl acetate/petroleum ether gradient @ 80 mL/min). tert-butyl N-[3-[(5-formyl-3-thienyl)methyl]phenyl]carbamate (130 mg, 410 μmol, 64.46% yield) was obtained as a yellow oil: MS m/z [M+H]⁺ (ESI): 340.1; ¹H NMR (400 MHz, DMSO-d₆) δ: 9.86 (s, 1H), 9.28 (s, 1H), 7.80 (s, 2H), 7.38 (s, 1H), 7.27 (d, J=8.0 Hz, 1H), 7.18 (t, J=7.6 Hz, 1H), 6.86 (d, J=7.6 Hz, 1H), 3.93 (s, 2H), 1.45 (s, 9H).

### Example 60. Preparation of (6aS,6bR,7S,8aS,8bS,10R,11aR,12aS,12bS)-6b-fluoro-10-((E)-2-(3-fluorothiophen-2-yl)vinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

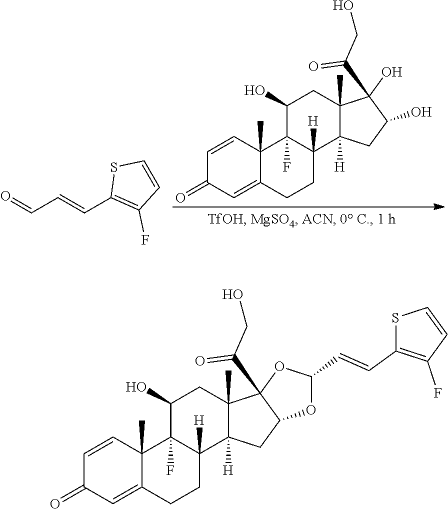

To a solution of triamcinolone (250 mg, 634 μmol, 1 eq) in CH₃CN (4 mL) was added MgSO₄ (382 mg, 3.17 mmol, 5 eq), (E)-3-(3-fluoro-2-thienyl)prop-2-enal (119 mg, 761 μmol, 1.2 eq) and TfOH (162 mg, 1.08 mmol, 95.0 uL, 1.70 eq) at 0° C. The mixture was stirred at 0° C. for 1 hr. The reaction mixture was quenched until pH=˜7 with NaHCO₃(aq) at 0° C., and then diluted with H₂O (10 mL) and extracted with DCM (10 mL×3). The combined organic layers were washed with brine (10 mL), dried over Na₂SO₄, filtered, and concentrated under reduced pressure to give a residue. The residue was purified by prep-HPLC (TFA condition, column: Phenomenex Luna 80×30 mm×3 um; mobile phase: [water(TFA)-ACN]; B %: 40%-60%, 8 min) to give (6aS,6bR,7S,8aS,8bS,10R,11 aR,12aS,12bS)-6b-fluoro-10-((E)-2-(3-fluorothiophen-2-yl)vinyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (84.2 mg, 158 μmol, 24.9% yield) as a white solid: MS m/z [M+H]⁺ (ESI): 533.3; ¹H NMR (400 MHz, DMSO-d₆) δ: 7.49 (t, J=4.8 Hz, 1H), 7.29 (d, J=10.0 Hz, 1H), 6.98 (d, J=2.8 Hz, 1H), 6.92 (d, J=16.0 Hz, 1H), 6.27-6.19 (m, 1H), 6.02 (s, 1H), 5.80-5.70 (m, 1H), 5.11 (d, J=6.0 Hz, 1H), 4.85 (d, J=4.4 Hz, 1H), 4.48 (d, J=19.2 Hz, 1H), 4.22-4.12 (m, 2H), 2.69-2.56 (m, 1H), 2.49-2.38 (m, 1H), 2.38-2.30 (m, 1H), 2.04-1.91 (m, 2H), 1.86-1.77 (m, 1H), 1.67-1.55 (m, 3H), 1.47 (s, 3H), 1.41-1.27 (m, 1H), 0.82 (s, 3H).

### Preparation of (E)-3-(3 fluoro-2-thienyl)prop-2-enal

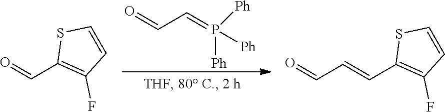

A mixture of 3-fluorothiophene-2-carbaldehyde (1.25 g, 9.61 mmol, 1 eq) and 2-(triphenyl-5-phosphanylidene)acetaldehyde (3.51 g, 11.5 mmol, 1.2 eq) in THE (26 mL) was degassed and purged with N2 for 3 times, and then stirred at 80° C. for 2 hr under N2 atmosphere. The reaction mixture was concentrated under reduced pressure. The residue was purified by flash silica gel chromatography (ISCO®; 4 g SepaFlash® Silica Flash Column, Eluent of 0-30% Ethyl acetate/Petroleum ether gradient @ 36 mL/min) to give (E)-3-(3-fluoro-2-thienyl)prop-2-enal (805 mg, 5.15 mmol, 53.7% yield) as yellow oil: ¹H NMR (400 MHz, DMSO-d₆) δ: 9.62 (d, J=7.6 Hz, 1H), 7.89 (t, J=4.8 Hz, 1H), 7.83 (d, J=15.6 Hz, 1H), 7.17 (d, J=5.6 Hz, 1H), 6.46-6.38 (m, 1H).

### Example 61. Preparation of (6aR,6bS,7S,8aS,8bS,10R,11aR,12aS,12bS)-10-(2-(3-fluorothiophen-2-yl)ethyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

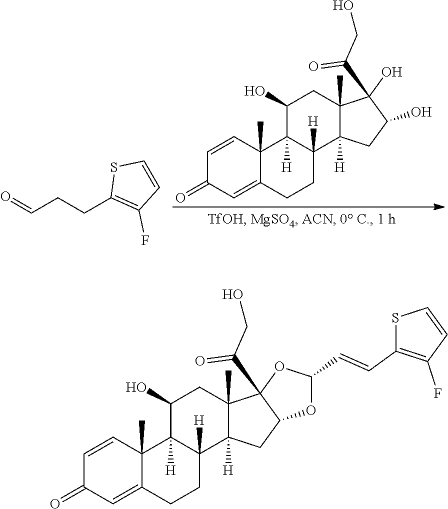

To a solution of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (99.2 mg, 263 μmol, 1 eq) in CH₃CN (1.5 mL) was added MgSO₄ (159 mg, 1.32 mmol, 5 eq), 3-(3-fluoro-2-thienyl)propanal (50 mg, 316 μmol, 1.2 eq) and TfOH (102 mg, 680 μmol, 60.0 uL, 2.58 eq) at 0° C. The mixture was stirred at 0° C. for 1 hr. The reaction mixture was quenched until pH=˜7 with NaHCO₃(aq) at 0° C., and then diluted with H₂O (5 mL) and extracted with DCM (5 mL×3). The combined organic layers were washed with brine (5 mL), dried over Na₂SO₄, filtered, and concentrated under reduced pressure to give a residue. The residue was purified by prep-HPLC (TFA condition, column: Phenomenex Luna 80×30 mm×3 um; mobile phase: [water(TFA)-ACN]; B %: 40%-60%, 8 min) to give (6aR,6bS,7S,8aS,8bS,10R,11aR,12aS,12bS)-10-(2-(3-fluorothiophen-2-yl)ethyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (26.0 mg, 50.2 μmol, 19.1% yield) as a white solid: MS m/z [M+H]⁺ (ESI): 517.3; ¹H NMR (400 MHz, DMSO-d₆) δ: 7.30 (d, J=10.0 Hz, 1H), 7.25-7.20 (m, 1H), 6.78 (d, J=5.6 Hz, 1H), 6.19-6.12 (m, 1H), 5.90 (s, 1H), 4.74 (d, J=4.4 Hz, 1H), 4.55 (t, J=4.0 Hz, 1H), 4.36 (d, J=19.2 Hz, 1H), 4.26-4.21 (m, 1H), 4.10 (d, J=19.2 Hz, 1H), 2.83-2.65 (m, 2H), 2.48-2.42 (m, 1H), 2.31-2.23 (m, 1H), 2.11-1.78 (m, 4H), 1.68-1.38 (m, 5H), 1.33 (s, 3H), 1.01-0.83 (m, 2H), 0.76 (s, 3H).

### Preparation of 3-(3 Fluoro-2-Thienyl)Propanal

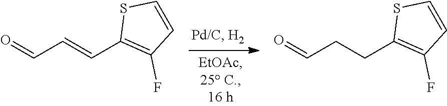 [SMILES: O=C/C=C/c1sccc1F ; O=CCCc1sccc1F]

A mixture of (E)-3-(3-fluoro-2-thienyl)prop-2-enal (300 mg, 1.92 mmol, 1 eq), Pd/C (0.1 g, 1.92 mmol, 10% purity, 1 eq) in EtOAc (6 mL) was degassed and purged with H₂ for 3 times, and then the mixture was stirred at 25° C. for 16 hrs under H₂ (15 psi) atmosphere. The reaction mixture was filtered with celite. The filter liquor was concentrated in vacuum to give a residue. The residue was purified by flash silica gel chromatography (ISCO®; 12 g SepaFlash® Silica Flash Column, Eluent of 0-30% Ethyl acetate/Petroleum ether gradient @ 40 mL/min) to give 3-(3-fluoro-2-thienyl)propanal (150 mg, 948 μmol, 49.4% yield) as green oil: ¹H NMR (400 MHz, DMSO-d₆) δ: 9.70 (s, 1H), 7.38-7.33 (m, 1H), 6.92 (d, J=5.6 Hz, 1H), 2.99-2.94 (m, 2H), 2.83-2.77 (m, 2H).

### Example 62. Preparation of (6aR,6bS,7S,8aS,8bS,10R,11aR,12aS,12bS)-10-((E)-3-aminostyryl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

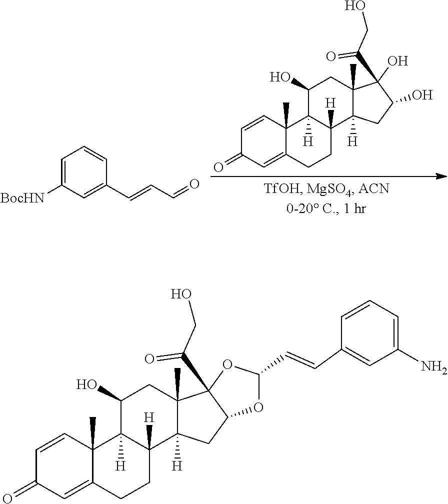

To a solution of (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (180 mg, 478 μmol, 1.0 eq) in MeCN (5.0 mL) was added MgSO₄ (230 mg, 1.91 mmol, 4.0 eq), tert-butyl N-[3-[(E)-3-oxoprop-1-enyl]phenyl]carbamate (177 mg, 717 μmol, 1.5 eq), TfOH (39.9 mg, 266 μmol, 23.5 uL, 5.0 eq) at 0° C. The mixture was stirred at 20° C. for 1 hour. The pH of mixture was adjusted to ˜8 with sat. NaHCO₃, water (10 mL) was added, then extracted with DCM (10 mL×3). The combined organic phase was dried with anhydrous Na₂SO₄, filtered, and concentrated in vacuum. The residue was purified by prep-HPLC(column: Phenomenex Luna 80×30 mm×3 um; mobile phase: [water(TFA)-ACN]; B %: 15%-35%, 8 min) to afford (6aR,6bS,7S,8aS,8bS,10R,11aR,12aS,12bS)-10-((E)-3-aminostyryl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (84 mg, 166 μmol, 34.7% yield) as yellow solid: MS m/z [M+H]⁺ (ESI): 506.4; ¹H NMR (400 MHz, DMSO-d₆) δ: 7.31 (d, J=9.6 Hz, 1H), 7.16-7.09 (m, 1H), 6.95-6.85 (m, 2H), 6.71 (d, J=16.0 Hz, 2H), 6.17 (d, J=1.6, 10.0 Hz, 1H), 6.07-6.02 (m, 1H), 5.93 (s, 1H), 5.08 (d, J=6.8 Hz, 1H), 4.85 (d, J=4.4 Hz, 1H), 4.82-4.74 (m, 1H), 4.51-4.49 (m, 1H), 4.31 (s, 1H), 4.17 (d, J=19.6 Hz, 1H), 2.35-2.27 (m, 2H), 2.16-1.96 (m, 2H), 1.85-1.69 (m, 2H), 1.68-1.53 (m, 3H), 1.39 (s, 3H), 1.10-0.96 (m, 2H), 0.84 (s, 3H).

### Preparation of tert-butyl N-[3-[(E)-3-oxoprop-1-enyl]phenyl]carbamate

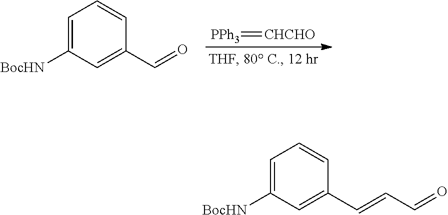

To a solution of tert-butyl N-(3-formylphenyl)carbamate (250 mg, 1.13 mmol, 1.0 eq) in THE (4 mL) was added 2-(triphenyl-λ⁵-phosphanylidene)acetaldehyde (413 mg, 1.36 mmol, 1.2 eq), and then stirred at 80° C. for 12 hours. The mixture was concentrated in vacuum. The residue was purified by silica gel chromatography (column height: 250 mm, diameter: 100 mm, 100-200 mesh silica gel, Petroleum ether/Ethyl acetate=1/0, 10/1) to afford tert-butyl N-[3-[(E)-3-oxoprop-1-enyl]phenyl]carbamate (250 mg, 1.01 mmol, 89.47% yield) as yellow oil: MS m/z [M+H]⁺ (ESI): 192.1.

### Example 63. Preparation of (6aR,6bS,7S,8aS,8bS,10R,11aR,12aS,12bS)-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one

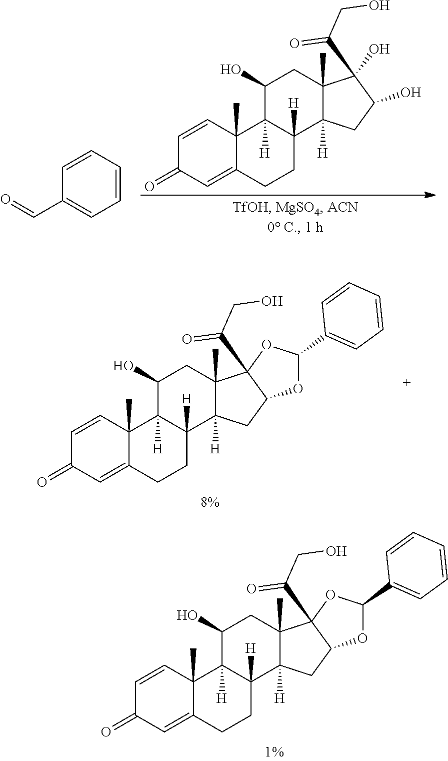

To a solution of benzaldehyde (200.0 mg, 1.88 mmol, 1.0 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (709.4 mg, 1.88 mmol, 1.0 equiv) in acetonitrile (2 mL) with an inert atmosphere of nitrogen, were added magnesium sulfate (160.3 mg, 6.59 mmol, 3.5 equiv) and trifluoromethanesulfonic acid (848.5 mg, 5.65 mmol, 3.0 equiv) dropwise at 0° C. The resulting solution was stirred for 1 h at 0° C. The resulting solution was purified by Flash with the following conditions: Column, C₁₈ silica gel; mobile phase, water and acetonitrile (5.0% acetonitrile up to 100.0% in 20 min); Detector, UV 254 nm. 19.4 mg (8.59%) of (6aR,6bS,7S,8aS,8bS, b OR, ii aR,12aS,12bS)-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 465.10; ¹H NMR (400 MHz, Methanol-d₄) δ: 0.99 (s, 3H), 1.00-1.15 (m, 2H), 1.49 (s, 3H), 1.70-1.82 (m, 4H), 1.90-2.00 (m, 1H), 2.10-2.20 (m, 1H), 2.25-2.35 (m, 2H), 2.60-2.70 (m, 1H), 4.30-4.35 (m, 1H), 4.40 (s, 1H), 4.60-4.65 (m, 1H), 5.06 (d, J=5.2 Hz, 1H), 5.46 (s, 1H), 6.02 (s, 1H), 6.20-6.25 (m, 1H), 7.36-7.38 (m, 3H), 7.44-7.46 (m, 3H).

11.9 mg (1.32%) of (6aR,6bS,7S,8aS,8bS,10S,11aR,12aS,12bS)-7-hydroxy-8b-(2-hydroxyacetyl)-6a,8a-dimethyl-10-phenyl-1,2,6a,6b,7,8,8a,8b,11a,12,12a,12b-dodecahydro-4H-naphtho[2′,1′:4,5]indeno[1,2-d][1,3]dioxol-4-one was obtained as a white solid: MS m/z [M+H]⁺ (ESI): 465.15; ¹H NMR (400 MHz, Methanol-d₄) δ: 1.01 (s, 3H), 1.12-1.15 (m, 2H), 1.50 (s, 3H), 1.75-1.79 (m, 2H), 1.82-1.93 (m, 2H), 2.01-2.05 (m, 1H), 2.18-2.23 (m, 2H), 2.41-2.42 (m, 1H), 2.60-2.70 (m, 1H), 4.08-4.13 (m, 1H), 4.27-4.31 (m, 1H), 4.42 (s, 1H), 5.39-5.41 (m, 1H), 6.03 (s, 1H), 6.13 (s, 1H), 6.24-6.27 (m, 1H), 7.29-7.35 (m, 5H), 7.45-7.50 (m, 1H).

### Example 64. Preparation of (6aR,7S,8aS,8bS,10R,11aR,12aS,12bS)-10-cyclobutyl-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

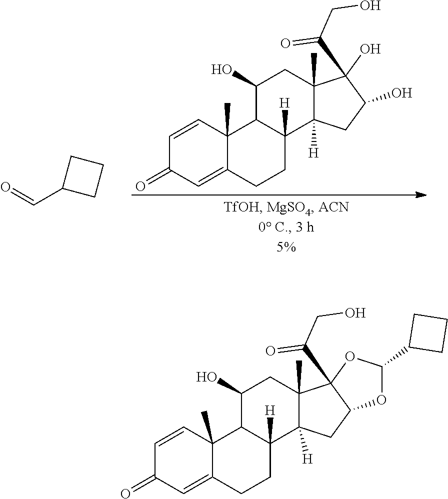

To a solution of cyclobutyral (500.0 mg, 5.94 mmol, 1.5 equiv) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (1.4 g, 3.96 mmol, 1.0 equiv), magnesium sulfate (1.6 g, 13.87 mmol, 3.5 equiv) in acetonitrile was added trifluoro(sulfonyl)methane (1.7 g, 11.8 mmol, 3.0 equiv) dropwise at 0° C. under nitrogen atmosphere. The mixture was basified to pH 7 with saturated sodium bicarbonate. The aqueous layer was extracted with methylene chloride (3×30 mL). The crude product was purified by Prep-HPLC with the following conditions (column, C₁₈ silica gel; mobile phase, acetonitrile in Water (0.1% ammonia+10 mmol/L ammonium bicarbonate) to afford (6aR,7S,8aS,8bS,10R,11aR,12aS,12bS)-10-cyclobutyl-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4, 5]indeno[1,2-d][1,3]dioxol-4-one (101.5 mg, 5%) as a white solid: MS m/z [M+H]⁺ (ESI): 443.15; ¹H NMR (300 MHz, Methanol-d₄) δ: 0.93-1.00 (m, 5H), 1.48 (s, 3H), 1.62-1.79 (m, 5H), 1.82-1.98 (m, 6H), 2.03-2.16 (m, 2H), 2.39-2.40 (m, 1H), 2.64-2.70 (m, 2H), 4.24-4.30 (m, 1H), 4.41-4.42 (m, 1H), 4.48-4.55 (m, 2H), 4.88-4.89 (m, 1H), 6.01 (s, 1H), 6.23-6.27 (m, 1H), 7.43-7.50 (m, 1H).

### Example 65. Preparation of (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

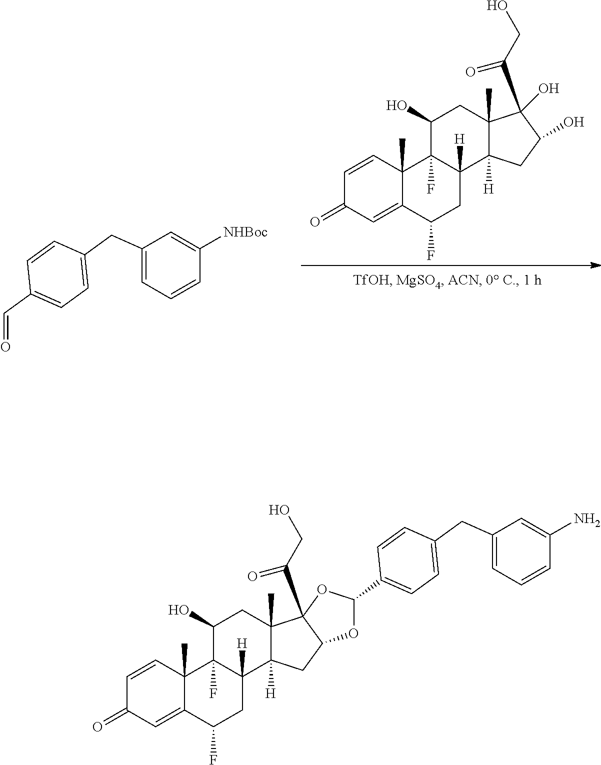

A mixture of fluocinolone (0.15 g, 363 μmol, 1 eq), tert-butyl N-[3-[(4-formylphenyl)methyl]phenyl]carbamate (169 mg, 545 μmol, 1.5 eq), MgSO₄ (218 mg, 1.82 mmol, 5 eq) in MeCN (3 mL) was degassed and purged with N2 for 3 times, then added TfOH (163 mg, 1.09 mmol, 96.3 uL, 3 eq) at 0° C., and then stirred at 0° C. for 1 hour under N2 atmosphere. The resulting solution was quenched with saturated aqueous solution of NaHCO₃ until pH˜7 and extracted with EtOAc (10 mL×2). The combined organic layers were washed with brine (10 mL), dried over Na₂SO₄, filtered, and concentrated under reduced pressure to give a residue. The residue was purified by prep-HPLC (column: Phenomenex Luna 80×30 mm×3 um; mobile phase: [water(TFA)-ACN]; B %: 25%-45%, 8 min). (2S,6aS,6bR,7S,8aS,8bS,10R,11aR,12aS,12bS)-10-(4-(3-aminobenzyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (135 mg, 222 μmol, 61.2% yield) was obtained as a white solid: MS m/z [M+H]⁺ (ESI): 606.2; ¹H NMR (400 MHz, methanol-d₄) δ: 7.40-7.21 (m, 6H), 7.11 (d, J=7.60 Hz, 1H), 7.03-6.94 (m, 2H), 6.30-6.28 (m, 1H), 6.13 (s, 1H), 5.75-5.52 (m, 1H), 5.44 (s, 1H), 4.93 (s, 1H), 4.49 (d, J=19.2 Hz, 1H), 4.49 (d, J=19.2 Hz, 1H), 4.20-4.15 (m, 2H), 3.92 (s, 2H) 2.75-2.57 (m, 1H), 2.36-2.27 (m, 1H), 2.22-2.19 (m, 1H), 2.00 (d, J=13.6 Hz, 1H) 1.76-1.64 (m, 3H), 1.52-1.43 (m, 4H), 0.85 (s, 3H).

### Preparation of tert-butyl N-[3-[(4 formylphenyl)methyl]phenyl]carbamate

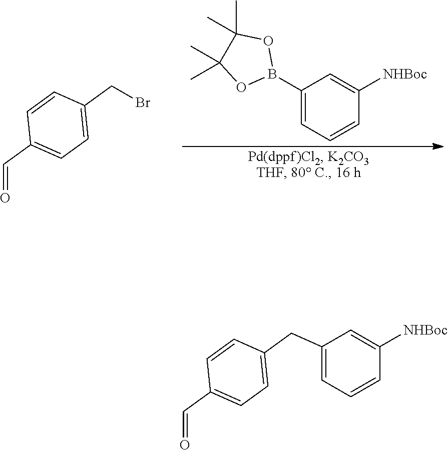

A mixture of tert-butyl N-[3-(4,4,5,5-tetramethyl-1,3,2-dioxaborolan-2-yl)phenyl]carbamate (1 g, 3.13 mmol, 1 eq), 4-(bromomethyl)benzaldehyde (935 mg, 4.70 mmol, 1.5 eq), Pd(dppf)Cl₂·CH₂Cl₂ (89.5 mg, 109 μmol, 0.035 eq), K₂CO₃ (2.16 g, 15.6 mmol, 5 eq) in THE (20 mL) was degassed and purged with N2 for 3 times, and then stirred at 80° C. for 16 hours under N₂ atmosphere. The reaction mixture was quenched by addition H₂O (50 mL) and extracted with EtOAc (50 mL×2). The combined organic layers were washed with brine (50 mL), dried over Na₂SO₄, filtered, and concentrated under reduced pressure to give a residue. The residue was purified by column chromatography (SiO₂, Petroleum ether/Ethyl acetate=100/1 to 1/1). tert-butyl N-[3-[(4-formylphenyl)methyl]phenyl]carbamate (0.7 g, 2.25 mmol, 71.8% yield) was obtained as a white solid: ¹H NMR (400 MHz, CDCl₃) δ: 9.89 (s, 1H), 7.72 (d, J=8.4 Hz, 2H), 7.27 (d, J=8.0 Hz, 2H), 7.18-7.11 (m, 3H), 6.77 (d, J=6.8 Hz, 1H), 6.40 (s, 1H), 3.94 (s, 2H), 1.42 (s, 9H).

### Example 66. Preparation of Di(ammonium) 2-(2-(2,5-Dioxo-2,5-dihydro-1H-pyrrol-1-yl)acetamido)ethyl diphosphato

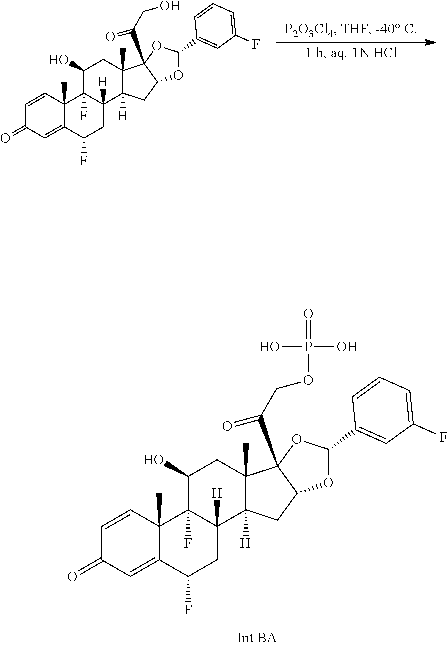

To a solution of starting material (800.0 mg, 1.54 mmol, 1.0 equiv.) in tetrahydrofuran (5.0 mL) with an inert atmosphere of nitrogen, was added pyrophosphoryl chloride (1.1 g, 4.62 mmol, 3.0 equiv.) dropwise at −40° C. The resulting solution was stirred for 1 h at −40° C. The reaction was quenched with water (5.0 mL), adjusted pH to 8 with saturated sodium bicarbonate solution (5.0 mL). The reaction was adjusted pH to 3 with hydrochloric acid (10.0 mL, 1.0 mol/L) and extracted with 3×300 mL of dichloromethane and the organic layers were combined, washed with 3×300 mL of saturated sodium chloride solution, dried over anhydrous sodium sulfate, filtered, and concentrated under reduced pressure. 650 mg (70%) of Int BA was obtained as white solid. MS m/z [M−H]⁻ (ESI): 597.15

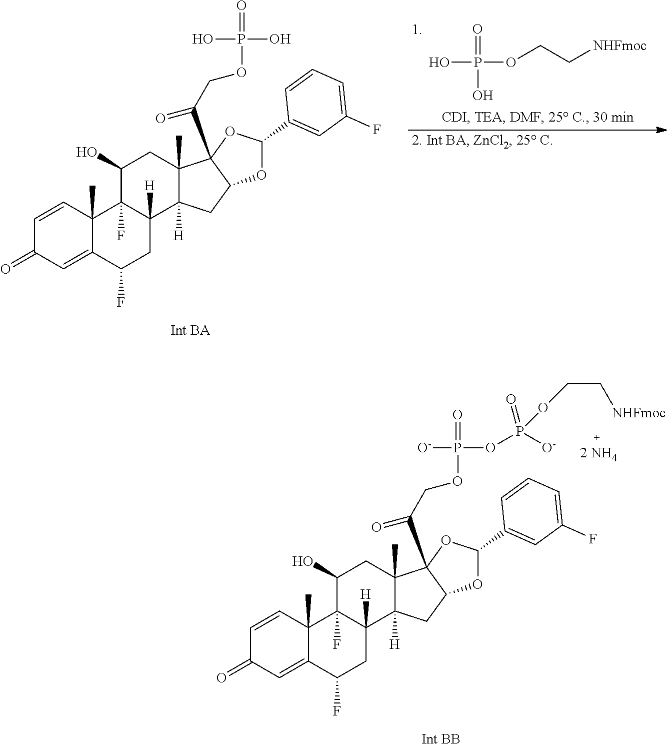

To a solution of (9H-fluoren-9-yl)methyl (2-(phosphonooxy)ethyl)carbamate (327.0 mg, 0.90 mmol, 1.5 equiv.) in N,N-dimethylformamide (5.0 mL) with an inert atmosphere of nitrogen, was added trimethylamine (91.0 mg, 0.90 mmol, 1.2 equiv.), 1,1′-carbonyldiimidazole (183.0 mg, 1.13 mmol, 1.5 equiv.). The resulting mixture was stirred for 30 min at 25° C. To this mixture was added Int BA (450.0 mg, 0.75 mmol, 1.0 equiv.) and zinc chloride (816.0 mg, 6.0 mmol, 8.0 equiv.). The resulting mixture was stirred for overnight at 25° C., diluted with methanol (10.0 mL) and filtered. The filtrate was concentrated under reduced pressure. The crude product was purified by Flash with the following conditions: Column, C₁₈ silica gel; mobile phase, water (with 5 mmol/L ammonium hydroxide) and acetonitrile (10.0% acetonitrile up to 100.0% in 20 min); Detector, UV 254 nm. 310 mg of Int BB was obtained as white solid. MS m/z [M−H]⁻ (ESI): 942.20

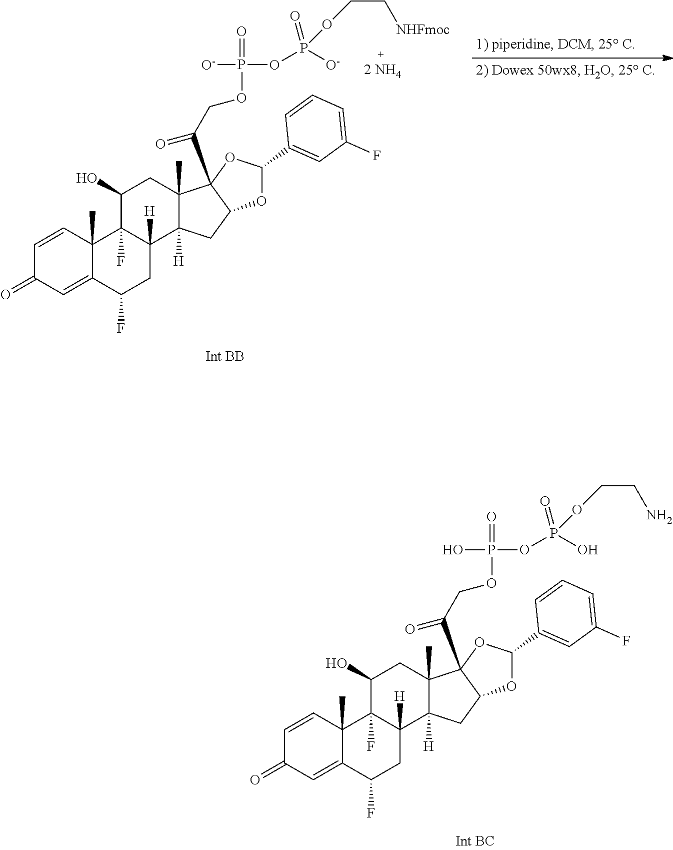

To a solution of Int BB (310.0 mg, 0.33 mmol, 1.0 equiv.) in dichloromethane (5.0 mL) with an inert atmosphere of nitrogen, was added piperidine (196.0 mg, 2.31 mmol, 7.0 equiv.).

The resulting solution was stirred for 3 h at 25° C., concentrated under reduced pressure. The crude product was purified by Flash with the following conditions: Column, C₁₈ silica gel; mobile phase, water (with 5 mmol/L ammonium hydroxide) and acetonitrile (5.0% acetonitrile up to 100.0% in 20 min); Detector, UV 254 nm. The eluent was concentrated under reduced pressure. To the solution of Int BC (ammonium salt) in water (5.0 mL) with an inert atmosphere of nitrogen, was added Dowex 50 w×8 (200.0 mg). The resulting solution was stirred for additional 1 h at 25° C., filtered. The filtrate was concentrated under reduced pressure. 115 mg (21% over two steps) of Int BC was obtained as white solid. MS m/z [M−H]⁻ (ESI): 720.20.

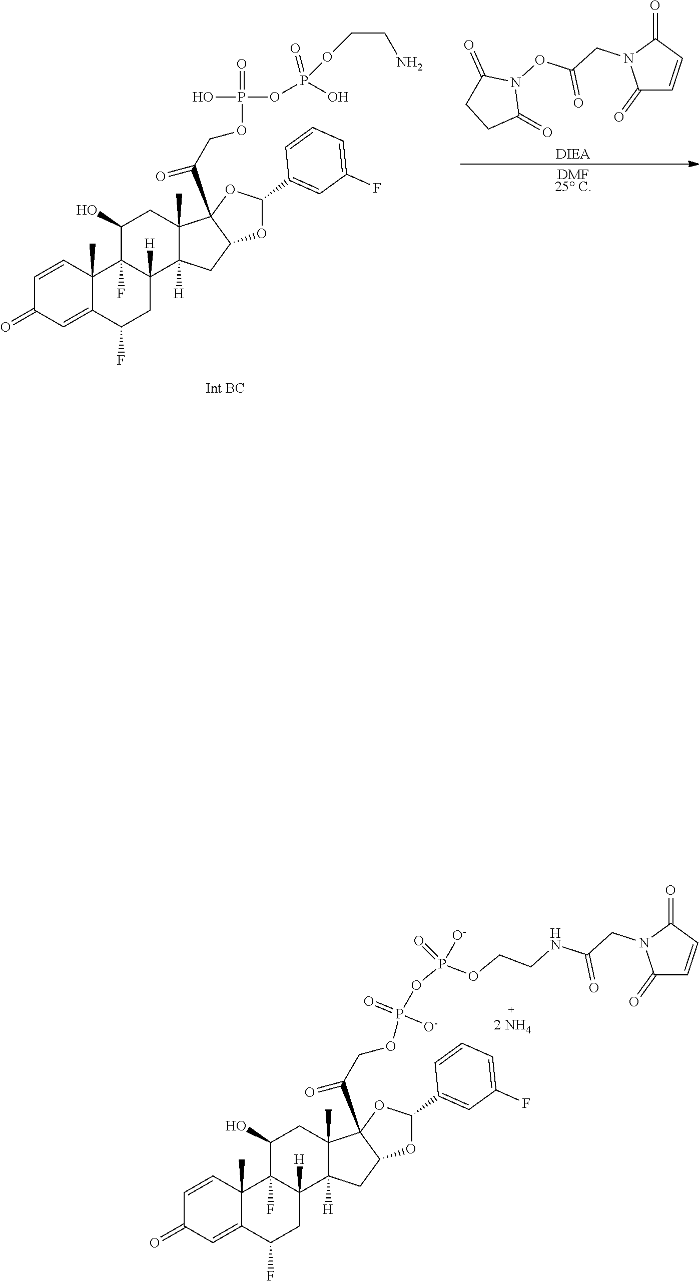

To a solution of Int BC (40.0 mg, 0.056 mmol, 1 equiv.) in N,N-dimethylformamide (1.0 mL) with an inert atmosphere of nitrogen, was added 2,5-dioxopyrrolidin-1-yl 2-(2,5-dioxopyrrol-1-yl)acetate (28.04 mg, 0.112 mmol, 2.0 equiv.) and N,N-diisopropylethylamine (21.55 mg, 0.168 mmol, 3.0 equiv.). The resulting solution was stirred for 6 h at 25° C. The crude product was purified by Prep-HPLC with the following conditions (IntelFlash-1): Column, XBridge Shield RP18 OBD Column, 19×250 mm, 10 μm; mobile phase, water (with 5.0 mmol/L ammonium bicarbonate) and acetonitrile (22.0% acetonitrile up to 28.0% in 12 min); Detector, UV 254 nm. 13.4 mg (27%) of desired compound was obtained as a white solid. MS m/z [M+H]⁺ (ESI): 859.16; ¹H NMR (400 MHz, Methanol-d₄) δ: 1.03 (s, 3H), 1.27-1.30 (m, 1H), 1.52-1.70 (m, 4H), 1.70-1.85 (m, 2H), 1.95-1.99 (m, 1H), 2.15-2.26 (m, 1H), 2.26-2.38 (m, 2H), 2.60-2.80 (m, 1H), 3.40-3.50 (m, 2H), 4.01-4.10 (m, 2H), 4.22-4.26 (m, 2H), 4.26-4.35 (m, 1H), 4.90-5.12 (m, 3H), 5.45-5.62 (m, 2H), 6.30-6.35 (m, 2H), 6.86 (s, 2H), 7.05-7.12 (m, 1H), 7.15-7.21 (m, 1H), 7.22-7.41 (m, 3H).

### Example 67. Preparation of GR Agonist-Linker Compounds

The compounds in Table 8 below were prepared in a manner similar to that described Example 66 using the appropriate compounds as starting material.

TABLE 8
GR Agonist-Linker Compounds

| Structure | MS/¹H NMR |
|---|---|
| 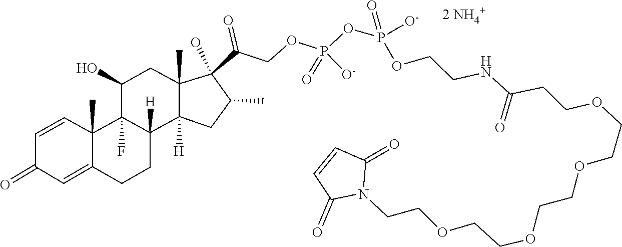 | MS m/z [M − H]⁻ (ESI): 921.35; ¹H NMR (400 MHz, Methanol- d₄) δ: 0.85-0.87 (d, J = 7.2 Hz, 3H), 1.01 (s, 3H), 1.11-1.21 (m, 1H), 1.46-1.58 (m, 1H), 1.59- 1.61 (s, 3H), 1.70-1.73 (m, 2H), 1.75-1.85 (m, 1H), 2.21-2.30 (m, 2H), 2.36-2.39 (m, 1H), 2.40-2.51 (m, 1H), 2.52-2.55 (t, 2H), 2.65-2.80 (m, 1H), 3.05- 3.15 (m, 1H), 3.42-3.45 (m, 2H), 3.56-3.59 (m, 13H), 3.61- 3.69 (m, 2H), 3.73-3.76 (m, 2H), 4.02-4.12 (s, 2H), 4.25- 4.27 (d, J = 9.6 Hz, 1H), 4.82- |
| | 4.85 (m, 2H), 5.02-5.06 (m, |
| | 1H), 6.06 (s, 1H), 6.29-6.30 (d, |
| | J = 2.0 Hz, 1H), 6.82 (s, 2H), |
| | 7.42-7.44 (d, J = 10.0 Hz, 1H). |
| 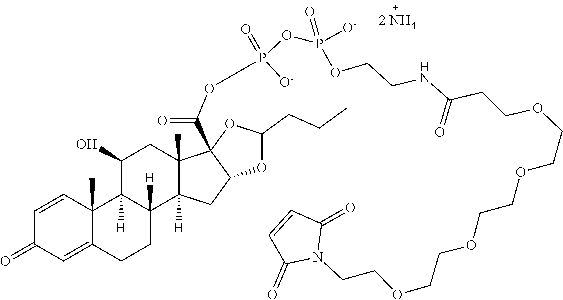 | MS m/z [M − H]⁻ (ESI): 959.35; ¹H NMR (400 MHZ, Methanol- d₄) δ: 0.89-1.00 (m, 6H), 1.05- 1.15 (m, 2H), 1.40-1.48 (m, 3H), 1.49 (s, 3H), 1.60-1.73 (m, 5H), 1.93-1.95 (m, 2H), 2.05- 2.15 (m, 2H), 2.35-2.49 (m, 1H), 2.51-2.52 (m, 2H), 2.60- 2.65 (m, 1H), 3.40-3.45 (m, 2H), 3.58-3.62 (m, 13H), 3.64- 3.67 (m, 2H), 3.68-3.74 (m, 2H), 4.01-4.04 (m, 2H), 4.43- 4.44 (m, 2H), 4.60-4.65 (m, 1H), 4.80-4.87 (m, 1H), 5.14- 5.20 (m, 1H), 6.00 (s, 1H), 6.25 (d, J = 10.0 Hz, 1H), 6.82 (s, 2H), 7.48 (d, J = 10.0 Hz, 1H). |
| 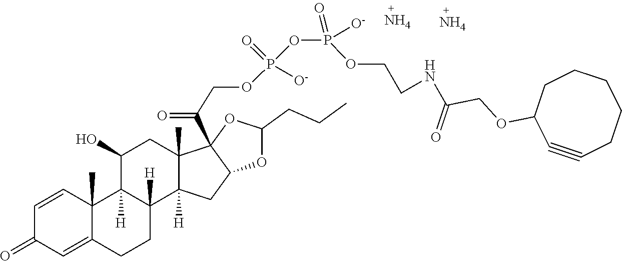 | MS m/z [M − H]⁻ (ESI): 796.15; ¹H NMR (400 MHZ, Methanol- d₄) δ: 0.91-1.02 (m, 7H), 1.43- 1.51 (m, 7H), 1.60-1.69 (m, 6H), 1.84-1.88 (m, 2H), 1.95- 2.14 (m, 4H), 2.16-2.19 (m, 5H), 2.25-2.30 (m, 1H), 2.60- 2.70 (m, 1H), 3.52-3.54 (m, 2H), 3.89-3.93 (m, 1H), 4.04- 4.08 (m, 3H), 4.32-4.33 (m, 1H), 4.45-4.50 (m, 1H), 4.67- 4.70 (m, 1H), 4.88-4.90 (m, 3H), 5.10-5.20 (m, 1H), 6.02 (s, 1H), 6.27 (d, J = 10.0 Hz, 1H), 7.50 (d, J = 10.0 Hz, 1H). |
| 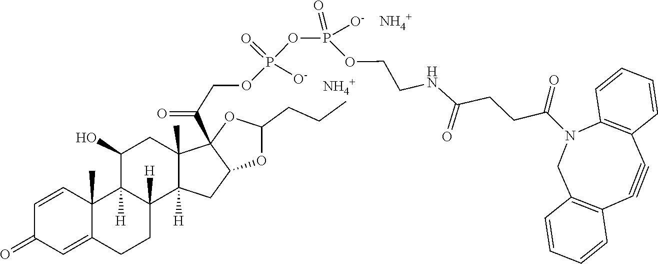 | MS m/z [M + H]⁺ (ESI): 921.31; ¹H NMR (400 MHZ, Methanol- d₄) δ: 0.87-1.00 (m, 8H), 1.39- 1.43 (m, 3H), 1.49-1.56 (m, 6H), 1.60-1.69 (m, 1H), 1.89- 1.96 (m, 3H), 2.10-2.15 (m, 1H), 2.20-2.30 (m, 2H), 2.38- 2.44 (m, 1H), 2.46-2.50 (m, 1H), 2.60-2.69 (m, 2H), 3.34- 3.35 (m, 2H), 3.68-3.71 (m, 1H), 3.97-3.98 (m, 2H), 4.41 (s, 1H), 4.60-4.63 (m, 1H), 4.86- 4.88 (m, 2H), 5.11-5.15 (m, 2H), 6.01 (s, 1H), 6.24-6.26 (m, 1H), 7.23-7.25 (m 1H), 7.31- 7.35 (m, 2H), 7.43-7.47 (m, |
| | 4H), 7.61-7.65 (m, 2H). |
| 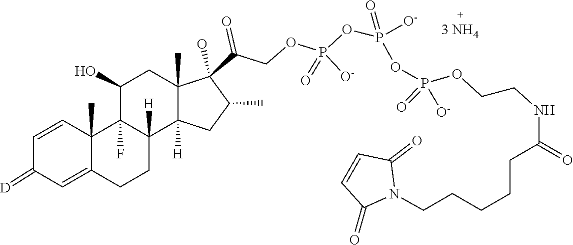 | MS m/z [M + H]⁺ (ESI): 869.25; ¹H NMR (400 MHZ, Methanol- d₄) δ: 0.86-0.88 (m, 3H), 1.02 (s, 3H), 1.19-1.20 (m, 1H), 1.29- 1.35 (m, 2H), 1.54-1.69 (m, 8H), 1.72-1.75 (m, 2H), 1.86- 1.87 (m, 1H), 2.22-2.27 (m, 4H), 2.32-2.39 (m, 2H), 2.71- 2.72 (m, 1H), 3.08-3.10 (m, 1H), 3.44-3.49 (m, 4H), 4.01- 4.06 (m, 2H), 4.26-4.28 (m, 1H), 4.70-4.78 (m, 1H), 4.81- 4.83 (m, 1H), 6.07 (s, 1H), 6.27- 6.29 (m, 1H), 6.78 (s, 2H), 7.43 (d, J = 10.0 Hz, 1H). |
| 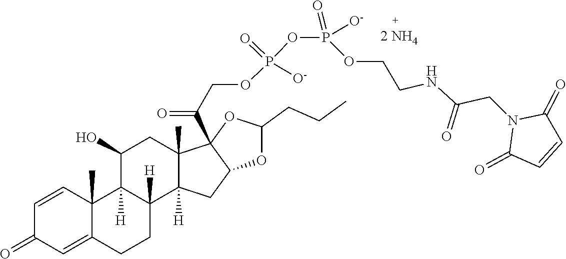 | MS m/z [M − H]⁻ (ESI): 769.00; ¹H NMR (400 MHZ, Methanol- d₄) δ: 0.88-0.92 (m, 6H), 0.97- 1.01 (m, 2H), 1.34-1.49 (m, 6H), 1.58-1.71 (m, 3H), 1.90- 2.00 (m, 2H), 2.01-2.12 (m, 4H), 2.30-2.40 (m, 1H), 2.60- 2.70 (m, 1H), 3.40-3.46 (m, 2H), 4.05-4.09 (m, 2H), 4.28 (s, 2H), 4.42 (s, 1H), 4.93-4.95 (m, 2H), 5.14-5.19 (m, 1H), 6.00 (s, 1H), 6.23-6.27 (m, 1H), 6.86 (s, 2H), 7.40-7.47 (m, 1H). |
| 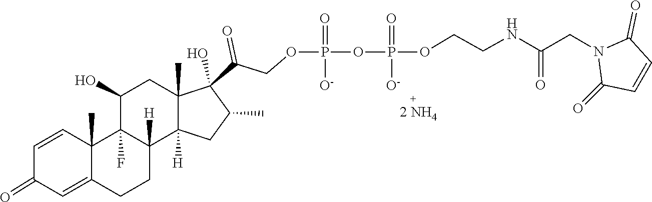 | MS m/z [M + H]⁺ (ESI): 733.30; ¹H NMR (400 MHZ, Methanol-d₄) δ: 0.86 (d, J = 7.2 Hz, 3H), 1.01 (s, 3H), 1.10-1.18 (m, 1H), 1.53-1.59 (m, 4H), 1.69-1.73 (m, 2H), 1.85-1.87 (m, 1H), 2.21-2.27 (m, 2H), 2.31-2.40 (m, 2H), 2.71-2.72 (m, 1H), 3.01-3.08 (m, 1H), 3.44-3.49 (m, 2H), 4.04-4.08 (m, 3H), 4.25-4.27 (m, 2H), 4.83-4.84 (m, 1H), |
| | 5.00-5.05 (m, 1H), 6.07 (s, |
| | 1H), 6.21-6.59 (m, 2H), 6.86 |
| | (s, 1H), 7.43 (d, J = 10.0 Hz, |
| | 1H). |
| 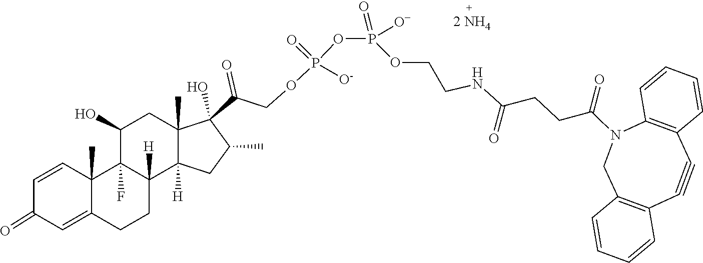 | MS m/z [M − H]⁻ (ESI): 881.25; ¹H NMR (400 MHZ, DMSO-d₆) δ: 0.76 (d, J = 6.8 Hz, 3H), 0.84 (s, 3H), 1.01-1.04 (m, 1H), 1.30- 1.37 (m, 1H), 1.47 (s, 3H), 1.57- 1.59 (m, 1H), 1.73-1.75 (m, 3H), 2.04-2.06 (m, 3H), 2.29- 2.39 (m, 3H), 2.60-2.67 (m, 1H), 2.93-3.00 (m, 1H), 3.10- 3.20 (m, 2H), 3.57-3.61 (m, 1H), 3.71-3.80 (m, 2H), 4.11- 4.14 (m, 1H), 4.60-4.70 (m, 1H), 5.02-5.05 (m, 1H), 5.99 (s, 1H), 6.18-6.19 (m, 1H), 6.95- 7.04 (m, 5H), 7.25-7.37 (m, 5H), 7.42-7.49 (m, 3H), 7.62- |
| | 7.63 (m, 1H), 7.71-7.75 (m, 1H) |
| 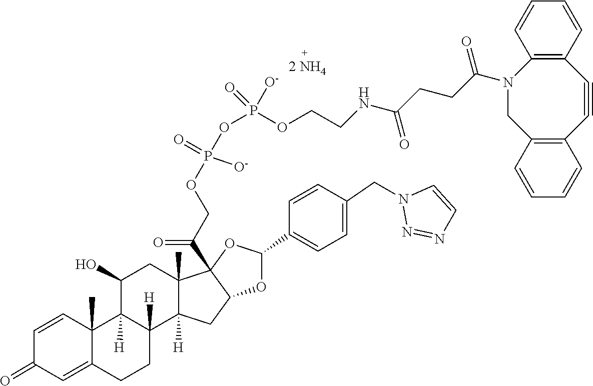 | MS m/z [M − H]⁻ (ESI): 1034.30; ¹H NMR (300 MHZ, DMSO-d₆) δ: 0.83-0.97 (m, 5H), 1.39 (s, 3H), 1.62-1.72 (m, 4H), 1.97- 2.05 (m, 3H), 2.27-2.31 (m, 1H), 2.57-2.59 (m, 1H), 3.00- 3.08 (m, 2H), 3.56-3.61 (m, 2H), 3.71-3.73 (m, 2H), 4.24 (s, 1H), 4.66-4.71 (m, 2H), 4.89 (s, 1H), 5.01-5.05 (m, 1H), 5.49 (s, 1H), 5.60 (s, 2H), 5.91 (s, 1H), 6.13 (d, J = 9.9 Hz, 1H), 7.27- 7.48 (m, 15H), 7.61-7.73 (m, 3H), 8.15 (s, 1H), 8.90 (s, 1H). |
| 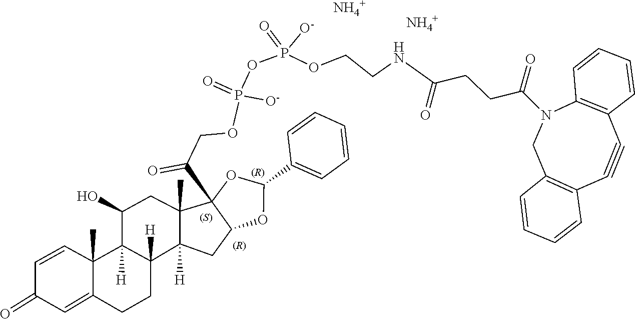 | MS m/z [M − H]⁻ (ESI): 953.35; ¹H NMR (400 MHZ, Methanol- d₄) δ: 1.00-1.05 (m, 4H), 1.10- 1.20 (m, 1H), 1.25-1.30 (m, 1H), 1.50 (s, 3H), 1.80-1.93 (m, 7H), 2.10-2.30 (m, 3H), 2.41- 2.50 (m, 2H), 2.66-2.69 (m, 2H), 3.66-3.69 (m, 1H), 3.97- 4.05 (m, 2H), 4.42 (s, 1H), 5.02- 5.13 (m, 4H), 5.53 (s, 1H), 6.02 (s, 1H), 6.24-6.26 (m, 1H), 7.22- 7.34 (m, 6H), 7.42-7.48 (m, 6H), 7.62-7.70 (m, 2H). |
| 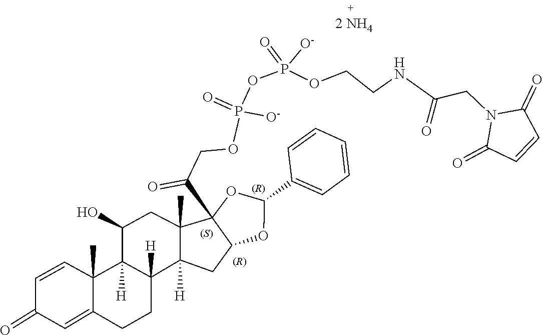 | MS m/z [M − H]⁻ (ESI): 803.25; ¹H NMR (400 MHZ, Methanol- d₄) δ: 1.00 (s, 3H), 1.50 (s, 3H), 1.71-1.84 (m, 4H), 1.93-1.99 (m, 2H), 2.01-2.15 (m, 1H), 2.24-2.27 (m, 1H), 2.37-2.39 (m, 1H), 2.67-2.69 (m, 1H), 2.96-2.99 (m, 1H), 3.31-3.43 (m, 2H), 4.05 (s, 2H), 4.12-4.23 (m, 3H), 4.44 (s, 1H), 5.00-5.05 (m, 2H), 5.57 (s, 1H), 6.01 (s, 1H), 6.23-6.26 (m, 1H), 6.84 (s, 2H), 7.35 (s, 3H), 7.47 (s, 3H). |
| 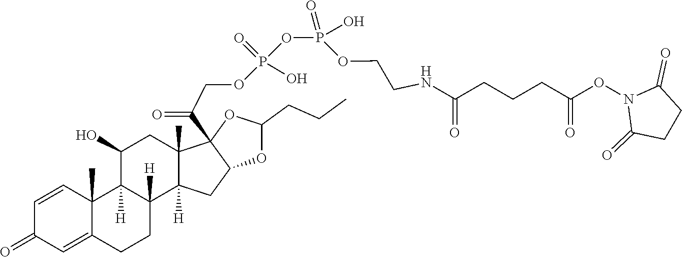 | MS m/z [M − H]⁻ (ESI): 843.30; ¹H NMR (400 MHZ, DMSO-d₆) δ: 0.83-0.88 (m, 7H), 1.14-1.39 (m, 25H), 1.51-1.57 (m, 3H), 1.60-1.69 (m, 2H), 1.80-1.84 (m, 2H), 1.96-1.98 (m, 3H), 2.21-2.28 (m, 4H), 2.59 (s, 1H), 2.66-2.69 (m, 2H), 2.80-2.85 (m, 5H), 3.04-3.17 (m, 7H), 3.59 (s, 2H), 3.78-3.79 (m, 2H), 4.25 (s, 1H), 4.50-4.56 (m, 1H), 4.70-4.72 (m, 1H), 5.17-5.20 (m, 1H), 5.60 (s, 1H), 5.91 (s, 1H), 6.13-6.16 (m, 1H), 7.29- 7.31 (m, 1H), 9.10-9.40 (m, 2H). |
| 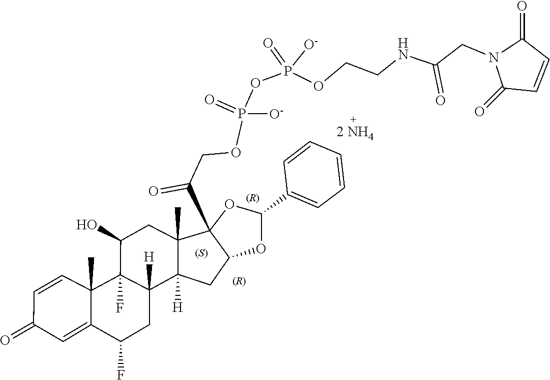 | MS m/z [M − H]⁻ (ESI): 839.20. ¹H NMR (400 MHZ, Methanol- d₄) δ: 1.03 (s, 3H), 1.58-1.76 (m, 4H), 1.78-1.80 (m, 2H), 1.93- 1.97 (m, 1H), 2.24-2.44 (m, 3H), 2.66-2.73 (m, 1H), 3.44- 3.47 (m, 2H), 4.03-4.07 (m, 2H), 4.24-4.35 (m, 3H), 4.98- 5.06 (m, 3H), 5.47-5.63 (m, 2H), 6.30-6.34 (m, 2H), 6.84 (s, 2H), 7.35-7.36 (m, 4H), 7.36- 7.46 (m, 2H). |
| 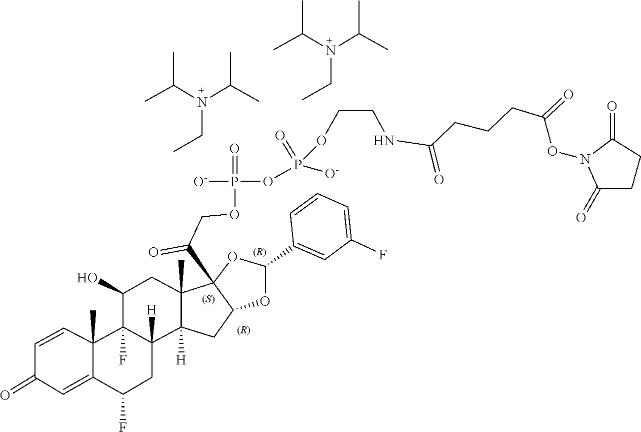 | MS m/z [M + H]⁺ (ESI): 933.30; ¹H NMR (300 MHz, Methanol- d₄) δ: 1.03 (s, 3H), 1.29-1.38 (m, 13H), 1.59-1.66 (m, 4H), 1.80- 1.87 (m, 2H), 1.96-2.03 (m, 3H), 2.12-2.39 (m, 4H), 2.53- 2.67 (m, 2H), 2.75-2.93 (m, 4H), 3.03-3.05 (m, 1H), 3.21- 3.23 (m, 2H), 3.43-3.69 (m, 2H), 3.72-3.74 (m, 1H), 4.07- 4.10 (m, 2H), 4.32-4.35 (m, 1H), 5.02-5.07 (m, 2H), 5.48- 5.60 (m, 2H), 6.31-6.36 (m, 2H), 7.11-7.22 (m, 2H), 7.33- 7.38 (m, 3H). |
| 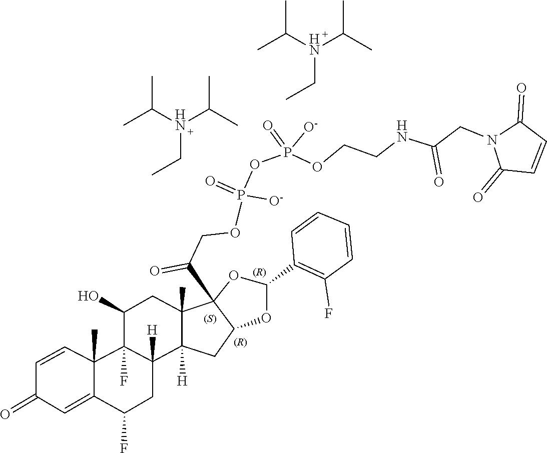 | MS m/z [M − H]⁻ (ESI): 857.00; ¹H NMR (300 MHz, Methanol- d₄) δ: 0.12 (s, 1H), 1.39 (s, 26H), 1.61 (s, 3H), 1.69-1.79 (m, 1H), 2.04-2.18 (m, 1H), 2.19-2.58 (m, 2H), 2.59-2.87 (m, 3H), 3.12-3.27 (m, 3H), 3.45 (s, 2H), 3.59-4.01 (m, 3H), 4.02-4.22 (m, 2H), 4.23-4.43 (m, 2H), 5.01-5.27 (m, 3H), 5.42-5.74 (m, 1H), 5.85 (s, 1H), 6.09-6.59 (m, 2H), 6.87 (s, 2H), 7.06-7.32 (m, 2H), 7.33-7.51 (m, 2H), 7.52-7.79 (m, 1H). |
| 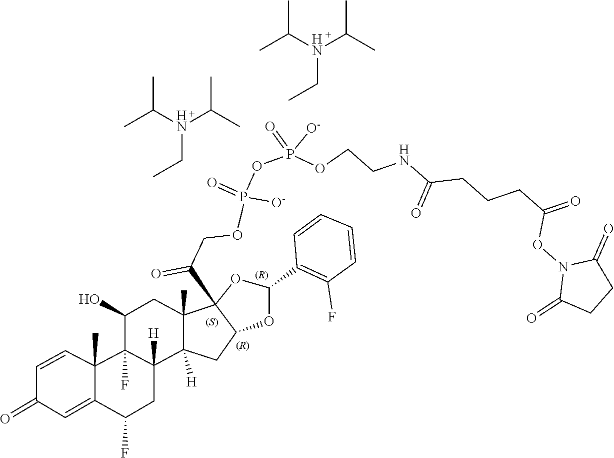 | MS m/z [M − H]⁻ (ESI): 931.15; ¹H NMR (400 MHZ, Methanol- d₄) δ: 1.00 (s, 3H), 1.40 (s, 26H), 1.79 (s, 3H), 1.81-1.99 (m, 1H), 2.01-2.08 (m, 3H), 2.24-2.41 (m, 4H), 2.70 (s, 3H), 2.76-2.86 (m, 6H), 3.21-3.24 (m, 3H), 3.33-3.45 (m, 1H), 3.71-3.75 (m, 3H), 4.09 (s, 2H), 4.34-4.37 (m, 1H), 5.02-5.10 (m, 2H), 5.50-5.65 (m, 1H), 5.84 (s, 1H), 6.32-6.38 (m, 2H), 7.09-7.16 (m, 1H), 7.20-7.25 (m, 1H), 7.37-7.41 (m, 2H), 7.61-7.63 (m, 1H) |
| 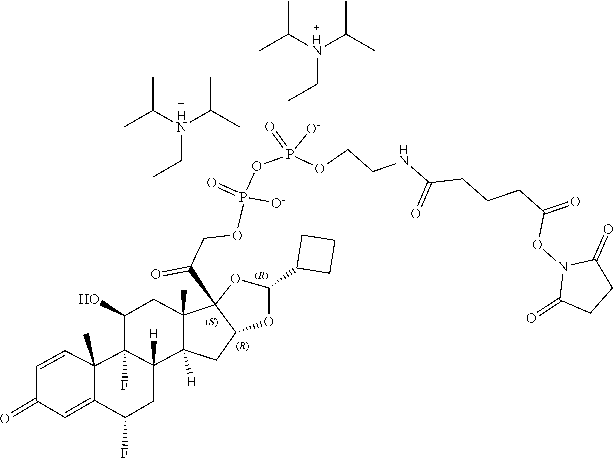 | MS m/z [M − H]⁻ (ESI): 891.15; ¹H NMR (300 MHz, Methanol- d₄) δ: 0.9 (s, 3H), 1.39 (s, 22H), 1.58 (s, 3H), 1.62-1.77 (m, 2H), 1.79-2.09 (m, 8H), 2.11-2.27 (m, 2H), 2.28-2.38 (m, 1H), 2.39-2.48 (m, 1H), 2.51-2.61 (m, 1H), 2.63-2.74 (m, 2H), 2.78-2.86 (m, 4H), 3.14-3.26 (m, 3H), 3.44 (s, 2H), 3.64-3.84 (m, 3H), 4.06-4.12 (m, 1H), 4.24-4.36 (m, 1H), 4.59-4.67 (m, 1H), 4.92-5.11 (m, 2H), 5.37-5.72 (m, 1H), 6.22-6.41 (m, 2H), 7.31-7.43 (m, 1H). |
| 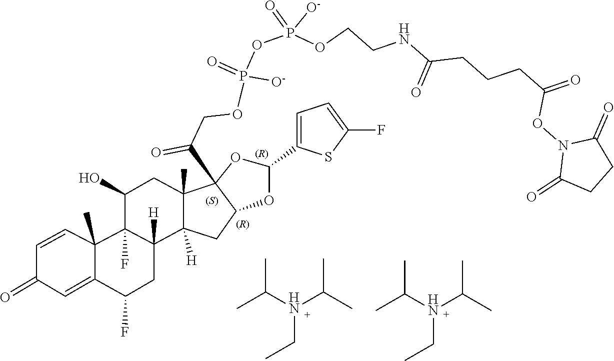 | MS m/z [M − H]⁻ (ESI): 937.10; ¹H NMR (400 MHZ, Methanol- d₄) δ: 0.99 (s, 3H), 1.29-1.38 (m, 27H), 1.59-1.68 (m, 4H), 1.73- 1.75 (m, 2H), 1.93-2.02 (m, 3H), 2.10-2.19 (m, 2H), 2.32- 2.42 (m, 4H), 2.68-2.71 (m, 3H), 2.81-2.84 (m, 7H), 3.18- 3.22 (m, 4H), 3.43 (s, 2H), 3.70- 3.73 (m, 3H), 4.07-4.09 (m, 2H), 4.31-4.33 (m, 1H), 5.02- 5.04 (m, 3H), 5.50-5.60 (m, 1H), 5.81 (s, 1H), 6.32-6.42 (m, 3H), 6.92 (s, 1H), 7.35-7.38 (m, 1H). |
| 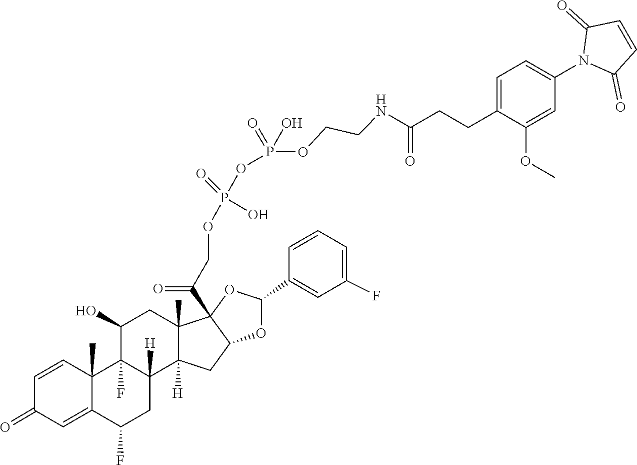 | MS m/z [M − H]⁻ (ESI): 977.30; ¹H NMR (300 MHz, Methanol- d₄) 0.99 (s, 3H), 1.56 -1.60 (m, 4H), 1.76-1.78 (m, 2H), 1.91 (d, J = 13.4 Hz, 1H), 2.31-2.34 (m, 3H), 2.49-2.53 (m, 2H), 2.84- 2.93 (m, 3H), 3.41-3.44 (m, 2H), 3.80 (s, 3H), 4.02-4.04 (m, 2H), 4.30-4.33 (m, 1H), 4.86- 4.99 (m, 1H), 5.02-5.05 (m, 2H), 5.50-5.59 (m, 2H), 6.30- 6.34 (m, 3H), 6.81-6.93 (m, 3H), 7.01-7.21 (m, 3H), 7.33- 7.38 (m, 3H). |
| 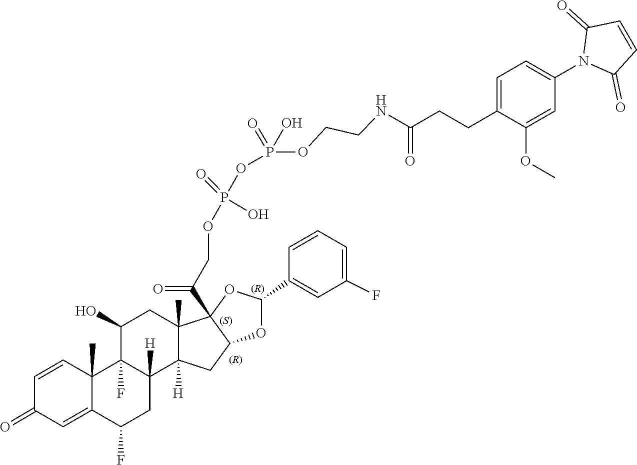 | MS m/z [M − H]− (ESI): 949.10; ¹H NMR (300 MHz, Methanol- d₄) δ: 0.99 (s, 3H), 1.20-1.30 (m, 1H), 1.57 (s, 4H), 1.70-1.78 (m, 2H), 1.90-2.00 (m, 1H), 2.15- 2.35 (m, 3H), 2.60-2.70 (m, 1H), 3.60-3.66 (m, 2H), 3.97- 3.99 (m, 3H), 4.13-4.15 (m, 2H), 4.30-4.35 (m, 1H), 5.01- 5.05 (m, 2H), 5.50-5.59 (m, 2H), 6.30-6.34 (m, 3H), 6.52- 6.60 (m, 1H), 7.16-7.34 (m, 6H), 7.61 (s, 1H), 7.89-7.92 (m, 1H). |

### Example 68. Preparation of 2-((6aR,6bS,7S,8aS,8bS,10R,11aR,12aS,12bS)-7-hydroxy-6a,8a-dimethyl-4-oxo-10-propyl-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl (3-(2,5-dioxo-2,5-dihydro-1H-pyrrol-1-yl)propanoyl)glycinate

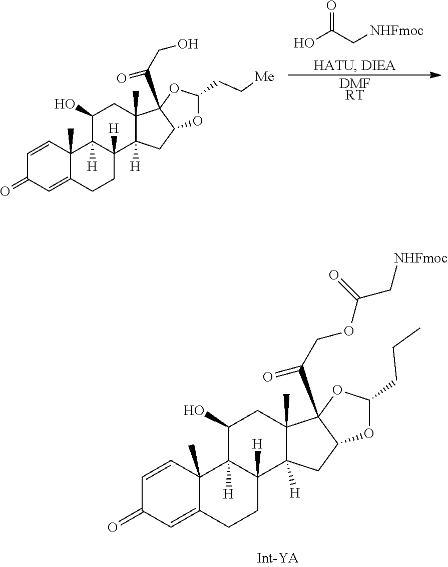

To a solution of budesonide (200.0 mg, 0.5 mmol, 1.0 equiv.) in N,N-Dimethylformamide (2.0 mL), was added {[(9H-fluoren-9-ylmethoxy)carbonyl]amino}acetic acid (207.2 mg, 0.7 mmol, 1.5 equiv.), O-Benzotriazole-N,N,N′,N′-tetramethyluronium hexafluorophosphate (352.3 mg, 0.9 mmol, 2.0 equiv.) and N,N-Diisopropylethylamine (180.1 mg, 1.4 mmol, 3.0 equiv.). The resulting solution was stirred for 5 h at 25° C., diluted with water (100 mL). The resulting mixture was extracted with dichloromethane (3×100 mL) and organic layers were washed with saturated sodium chloride solution (3×100 mL), dried over anhydrous sodium sulfate, filtration and concentrated under reduced pressure. The crude product was purified by Flash with the following conditions (IntelFlash-1): Column, C₁₈ silica gel; mobile phase, water and acetonitrile (10.0% acetonitrile up to 100.0% in 20 min); Detector, UV 254 nm. 210 mg (64%) of Int-YA was obtained as a white solid. MS m/z [M+H]⁺ (ESI): 710.33.

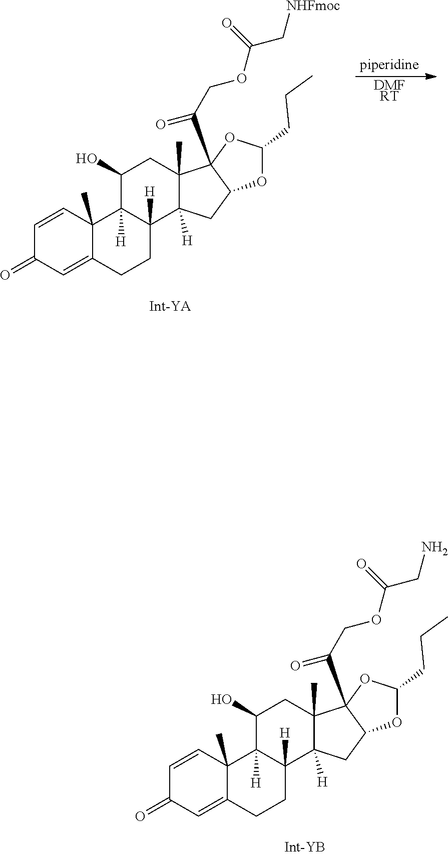

To a solution of Int-YA 110.0 mg, 0.15 mmol, 1.0 equiv. in dichloromethane 2.0 mL) with an inert atmosphere of nitrogen, was added piperidine (92.3 mg, 1.1 mmol, 7.0 equiv.). The resulting solution was stirred for additional 2 h at 25° C., concentrated under reduced pressure. The crude product was purified by Flash-Prep-HPLC with the following conditions (IntelFlash-1): Column, C₁₈ silica gel; mobile phase, water (with 5 mmol/L ammonium hydroxide) and acetonitrile (5.0% acetonitrile up to 100.0% in 20 min); Detector, UV 254 nm. 45 mg (59%) of

Int-YB was obtained as a white solid. MS m/z [M+H]⁺ (ESI):488.26.

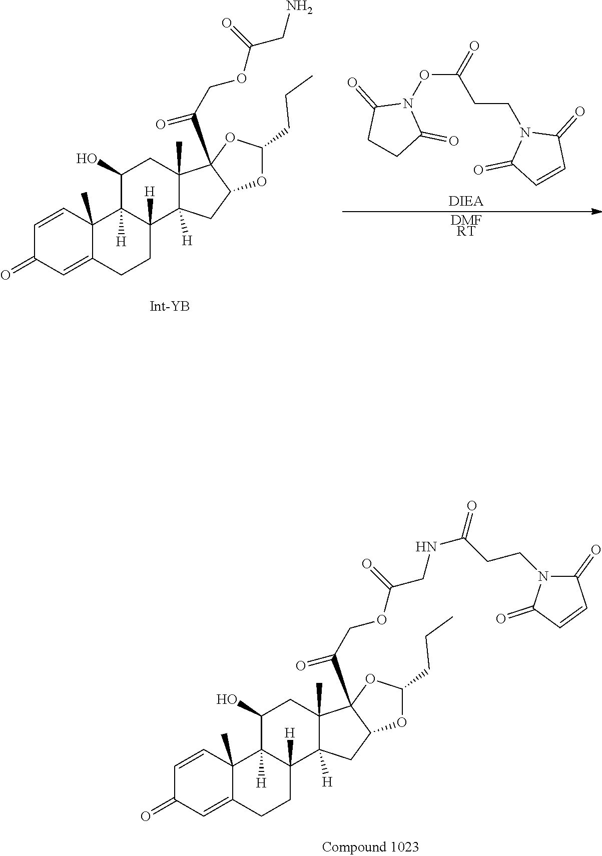

To a solution of Int-YB (45 mg, 0.1 mmol, 1.0 equiv.) N,N-dimethylformamide (1.0 mL) with an inert atmosphere of nitrogen, was added N,N-diisopropylethylamine (35.8 mg, 0.3 mmol, 3.0 equiv.) and 2,5-dioxopyrrolidin-1-yl 3-(2,5-dioxo-2,5-dihydro-1H-pyrrol-1-yl)propanoate(49 mg, 0.2 mmol, 2.0 equiv.). The resulting mixture was stirred for 3 h at 25° C. The crude product was purified by Prep-HPLC with the following conditions (IntelFlash-1): Column, YMC-Actus Triart C₁₈ ExRS, 30×150 mm, 5 μm; mobile phase, water and acetonitrile (10.0% acetonitrile up to 50.0% in 8 min); Detector, UV 254 nm. The resulting mixture was lyophilized in a cool and dry place. 16.7 mg (27%) of desired product was obtained as a white solid. MS m/z [M+H]⁺ (ESI): 639.20; ¹H NMR (400 MHz, Methanol-d₄) δ: 0.91-0.94 (m, 6H), 1.00-1.04 (m, 2H), 1.30-1.35 (m, 2H), 1.40 (s, 3H), 1.59-1.66 (m, 4H), 1.70-1.72 (m, 1H), 1.89-1.95 (m, 2H), 2.10-2.25 (m, 2H), 2.30-2.40 (m, 1H), 2.53-2.60 (m, 3H), 3.77-3.80 (m, 2H), 4.01-4.03 (m, 2H), 4.43 (s, 1H), 4.65-4.67 (m, 1H), 4.78-4.79 (m, 1H), 4.88-4.92 (m, 1H), 4.99-5.04 (m, 1H), 6.02 (s, 1H), 6.24-6.27 (m, 1H), 6.80 (s, 2H), 7.40-7.44 (m, 1H).

### Example 69. Preparation of GR Agonist-Linker Compounds

The compounds in Table 9 below were prepared in a manner similar to that described Example 68 using the appropriate compounds as starting material.

TABLE 9
GR Agonist-Linker Compounds

| Structure | MS/¹H NMR |
|---|---|
| 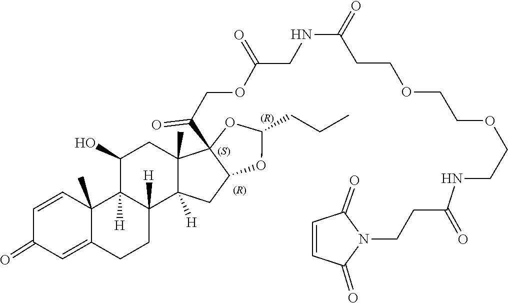 | MS m/z [M + Na]⁺ (ESI): 820.25; ¹H NMR (400 MHZ, DMSO-d₆) δ: 0.84-0.88 (m, 6H), 0.94-0.97 (m, 2H), 1.32- 1.38 (m, 5H), 1.52-1.61 (m, 5H), 1.81 (s, 2H), 1.98-2.07 (m, 2H), 2.28-2.33 (m, 3H), 2.35-2.40 (m, 2H), 2.50-2.55 (m, 1H), 3.12-3.16 (m, 2H), 3.34-3.36 (m, 2H), 3.37-3.60 (m, 8H), 3.95-3.99 (m, 2H), 4.31 (s, 1H), 4.64-4.65 (m, 2H), 4.71-4.84 (m, 2H), 5.06 (d, J = 18.0 Hz, 1H), 5.92 (s, 1H), 6.17 (d, J = 10.4 Hz, 1H), 6.99 (s, 2H), 7.31-7.33 (m, 1H), 7.99 (s, 1H), 8.34- 8.36 (m, 1H). |
| 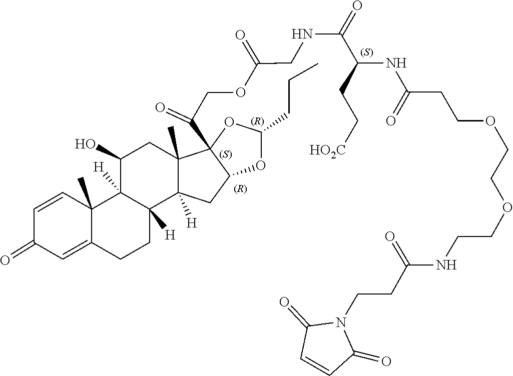 | MS m/z [M + H]⁺ (ESI): 927.50; ¹H NMR (400 MHZ, Methanol-d₄) δ: 0.90-1.10 (m, 8H), 1.42-1.49 (m, 5H), 1.59- 1.63 (m, 5H), 1.89-1.94 (m, 3H), 2.10-2.20 (m, 3H), 2.41- 2.54 (m, 7H), 2 |
| 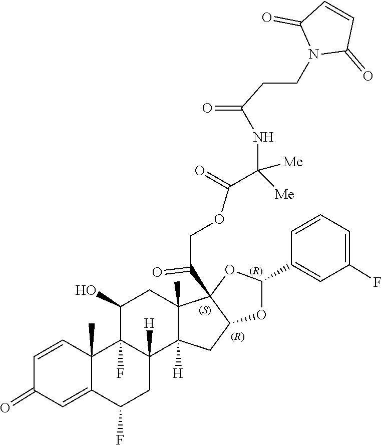 | MS m/z [M + H]⁺ (ESI): 755.35; ¹H NMR (400 MHZ, Methanol-d₄) δ: 1.01 (s, 3H), 1.43-1.51 (m, 6H), 1.58-1.65 (m, 4H), 1.77-1.90 (m, 3H), 2.26-2.38 (m, 3H), 2.41-2.49 (m, 2H), 2.66-2.73 (m, 1H), 3.73-3.76 (m, 2H), 4.32-4.34 (m, 1H), 4.89-4.94 (m, 1H), 5.02-5.10 (m, 2H), 5.49-5.62 (m, 2H), 6.32-6.35 (m, 2H), 6.79 (s, 2H), 7.11-7.21 (m, 2H), 7.30-7.42 (m, 3H). |
| 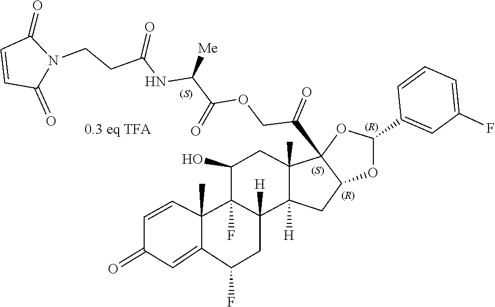 | MS m/z [M − H]⁻ (ESI): 739.35; ¹H NMR (400 MHZ, Methanol-d₄) δ: 1.01 (s, 3H), 1.47 (d, J = 7.6 Hz, 3H), 1.58- 1.65 (m, 4H), 1.78-1.90 (m, 3H), 2.26-2.37 (m, 3H), 2.50- 2.54 (m, 2H), 2.65-2.75 (m, 1H), 3.75-3.80 (m, 2H), 4.32- 4.34 (m, 1H), 4.46-4.48 (m, 1H), 5.01-5.06 (m, 3H), 5.50- 5.61 (m, 2H), 6.32-6.35 (m, 2H), 6.81 (s, 2H), 7.11-7.21 (m, 2H), 7.29-7.34 (m, 2H), 7.38-7.42 (m, 1H). |
| 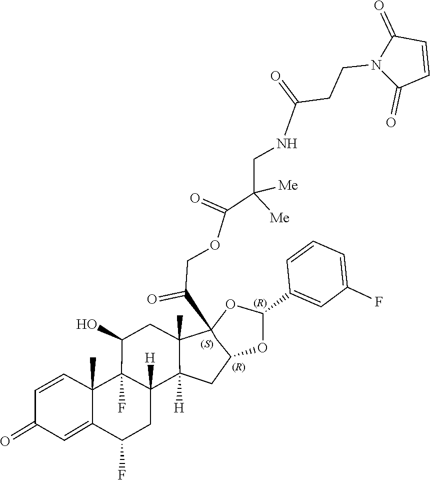 | MS m/z [M − H]⁻ (ESI): 767.30; ¹H NMR (300 MHZ, Methanol-d₄) δ: 1.03 (s, 3H), 1.21 (s, 6H), 1.59-1.66 (m, 4H), 1.81-1.91 (m, 3H), 2.27- 2.39 (m, 3H), 2.53-2.58 (m, 2H), 2.65-2.75 (m, 1H), 3.39 (s, 2H), 3.76-3.81 (m, 2H), 4.32-4.35 (m, 1H), 4.94-5.19 (m, 3H), 5.50-5.65 (m, 2H), 6.32-6.36 (m, 2H), 6.79 (s, 2H), 7.11-7.22 (m, 2H), 7.31- 7.43 (m, 3H). |
| 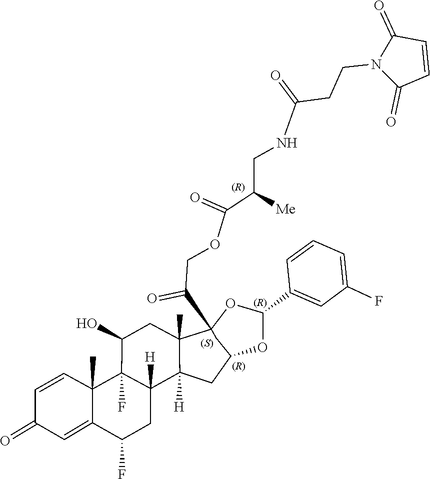 | MS m/z [M − H]⁻ (ESI): 753.30; ¹H NMR (400 MHZ, Methanol-d₄) δ: 1.02 (s, 3H), 1.19 (d, J = 7.2 Hz, 3H), 1.59- 1.62 (m, 4H), 1.79-1.89 (m, 3H), 2.27-2.38 (m, 3H), 2.48- 2.52 (m, 2H), 2.78-2.81 (m, 2H), 3.31-3.39 (m, 2H), 3.76- 3.79 (m, 2H), 4.32-4.34 (m, 1H), 4.96 (d, J = 17.6 Hz, 1H), 5.04 (s, 1H), 5.15 (d, J = 17.6 Hz, 1H), 5.50-5.63 (m, 2H), 6.32-6.35 (m, 2H), 6.79 (s, 2H), 7.13-7.21 (m, 2H), 7.30-7.40 (m, 3H). |

### Example 70. Preparation of 4-(2-((7S,10S)-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-7,10-dimethyl-1,6,9,12-tetraoxo-3-oxa-5,8,11-triazatetradecan-14-yl)-5-(2,5-dioxo-2,5-dihydro-1H-pyrrol-1-yl)phenoxy)butanoic acid

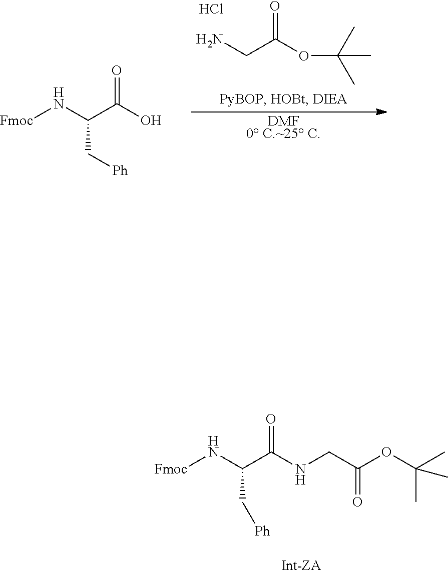

To a solution of (2S)-2-{[(9H-fluoren-9-ylmethoxy)carbonyl]amino}-3-phenylpropanoic acid (5.0 g, 12.9 mmol, 1.0 equiv.) in N,N-dimethylformamide (50.0 mL) with an inert atmosphere of nitrogen, was added tert-butyl 2-aminoacetate hydrochloride (2.6 g, 15.5 mmol, 1.2 equiv.), benzotriazol-1-yl-oxytripyrrolidinophosphonium hexafluorophosphate (10.1 g, 19.4 mmol, 1.5 equiv.), 1-hydroxybenzotriazole (2.6 g, 19.4 mmol, 1.5 equiv.), N,N-diisopropylethylamine (4.9 g, 38.7 mmol, 3.0 equiv.) at 0° C. The resulting solution was stirred for 4 h at 25° C., diluted with water (1000 mL). The resulting mixture was extracted with dichloromethane (3×1000 mL) and organic layers were washed with saturated sodium chloride solution (3×1000 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5.2 g (80%) of Int-ZA was obtained as white solid. MS m/z [M+H]⁺ (ESI): 501.23

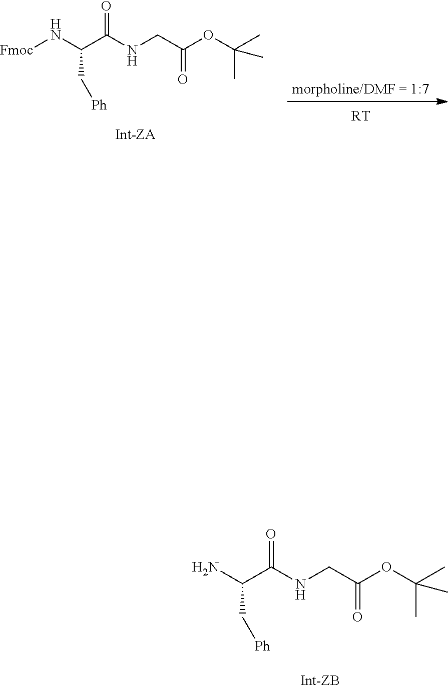

To a solution of Int-ZA (5.2 g, 10.4 mmol, 1.0 equiv.) in morpholine/N,N-dimethylformamide (7.0 mL/49.0 mL) with an inert atmosphere of nitrogen, the resulting solution was stirred for 1 h at 25° C., diluted with water (1000 mL). The resulting mixture was extracted with dichloromethane (3×1000 mL) and organic layers were washed with saturated sodium chloride solution (3×1000 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 2.7 g (93%) of Int-ZB was obtained as white solid. MS m/z [M+H]⁺ (ESI): 279.16.

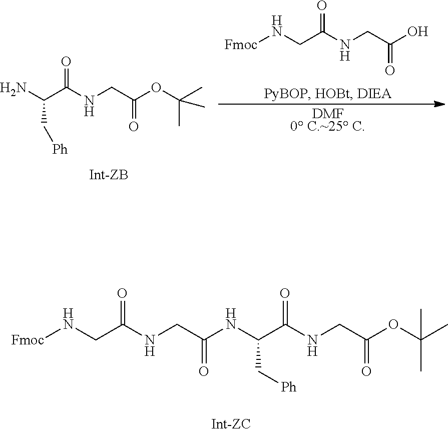

To a solution of Int-ZB (2.7 g, 9.7 mmol, 1.0 equiv.) in N,N-dimethylformamide (30.0 mL) with an inert atmosphere of nitrogen, was added (2-{[(9H-fluoren-9-ylmethoxy)carbonyl]amino}acetamido)acetic acid (3.4 g, 9.7 mmol, 1.0 equiv.), benzotriazol-1-yl-oxytripyrrolidinophosphonium hexafluorophosphate (7.6 g, 14.6 mmol, 1.5 equiv.), 1-hydroxybenzotriazole (1.9 g, 14.6 mmol, 1.5 equiv.), N,N-diisopropylethylamine (3.8 g, 29.1 mmol, 3.0 equiv.) at 0° C. The resulting solution was stirred for 4 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3.8 g (64%) of Int-ZC was obtained as white solid. MS m/z [M+H]⁺ (ESI): 615.27.

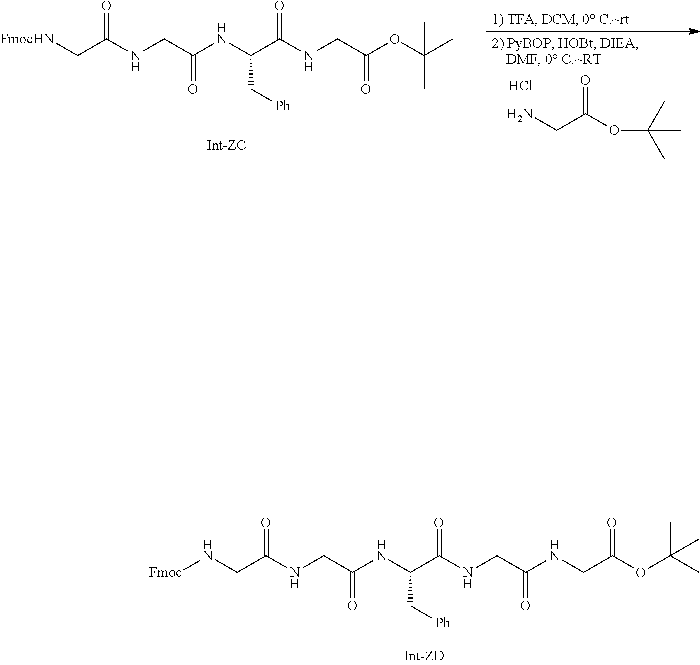

To a solution of Int-ZC (3.8 g, 6.2 mmol, 1.0 equiv.) in dichloromethane (40.0 mL) with an inert atmosphere of nitrogen, was added trifluoroacetic acid (10.0 mL) at 0° C. The resulting solution was stirred for 5 h at 25° C. and concentrated under vacuum. To this solution in N,N-dimethylformamide (30.0 mL) was added tert-butyl 2-aminoacetate hydrochloride (1.0 g, 6.2 mmol, 1.0 equiv.), benzotriazol-1-yl-oxytripyrrolidinophosphonium hexafluorophosphate (4.8 g, 9.3 mmol, 1.5 equiv.), 1-hydroxybenzotriazole (1.3 g, 9.3 mmol, 1.5 equiv.), N,N-diisopropylethylamine (2.4 g, 18.6 mmol, 3.0 equiv.) at 0° C. The resulting solution was stirred for 4 h at 25° C., diluted with water (300 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 2.6 g (64%) of Int-ZD was obtained as white solid. MS m/z [M+H]⁺ (ESI): 672.30; ¹H NMR (300 MHz, DMSO-d₆) δ: 1.39 (s, 9H), 2.75-2.82 (m, 1H), 3.03-3.09 (m, 1H), 3.56-3.80 (m, 8H), 4.19-4.31 (m, 3H), 4.49-4.57 (m, 1H), 7.15-7.24 (m, 7H), 7.25-7.44 (m, 2H), 7.57-7.61 (m, 1H), 7.69-7.72 (m, 2H), 7.88-7.99 (m, 2H), 8.01-8.15 (m, 3H), 8.31-8.35 (m, 1H).

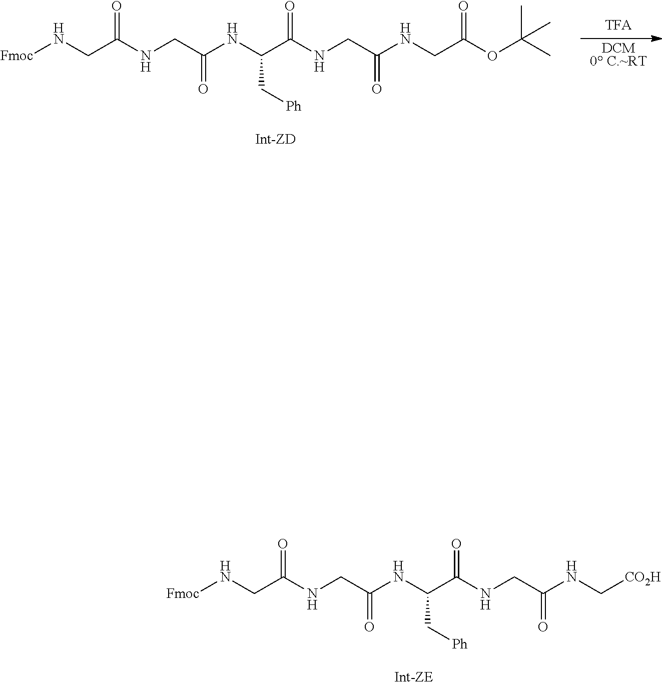

To a solution of Int-ZD (2.6 g, 3.9 mmol, 1.0 equiv.) in dichloromethane (30.0 mL) with an inert atmosphere of nitrogen, was added trifluoroacetic acid (15.0 mL) at 0° C. The resulting solution was stirred for 5 h at 25° C. and concentrated under vacuum. The crude product was purified by Flash-Prep-HPLC with the following conditions (IntelFlash-1): Column, C₁₈ silica gel; mobile phase, water (with 0.1% trifluoroacetic acid) and acetonitrile (10.0% acetonitrile up to 100.0% in 20 min); Detector, UV 254 nm. 2.1 g (88%) of Int-ZE was obtained as white solid. MS m/z [M+H]⁺ (ESI): 616.20.

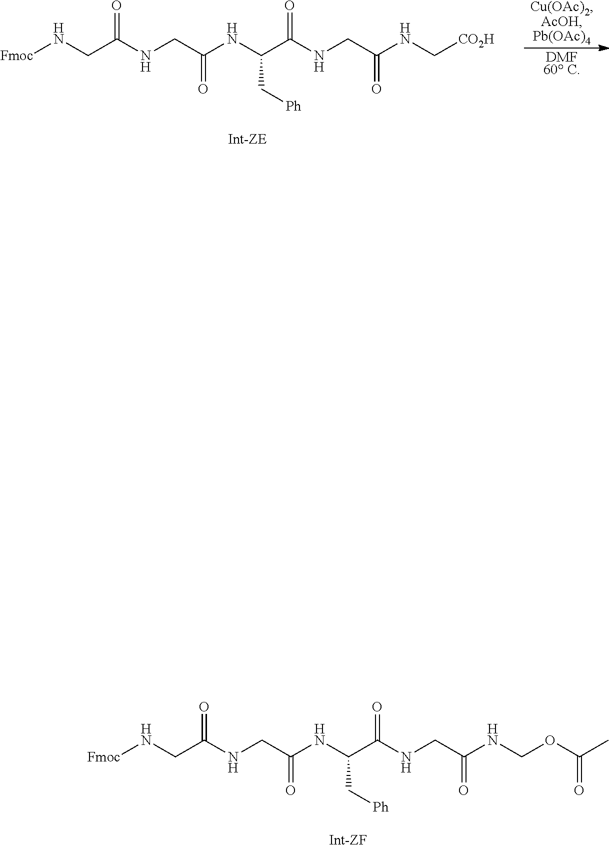

To a solution of Int-ZE (2.1 g, 3.4 mmol, 1.0 equiv.) in N,N-dimethylformamide (30.0 mL) with an inert atmosphere of nitrogen, was added cupric acetate anhydrous (254 mg, 1.4 mmol, 0.4 equiv.), acetic acid (468 mg, 7.8 mmol, 2.3 equiv.), lead tetraacetate (1.8 g, 4.1 mmol, 1.2 equiv.) at 25° C. The resulting mixture was stirred for 5 h at 60° C. The mixture was cooled down to 25° C., diluted with water (300 mL), extracted dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 870 mg (41%) of Int-ZF was obtained as white solid. MS m/z [M+H]⁺ (ESI): 630.20.

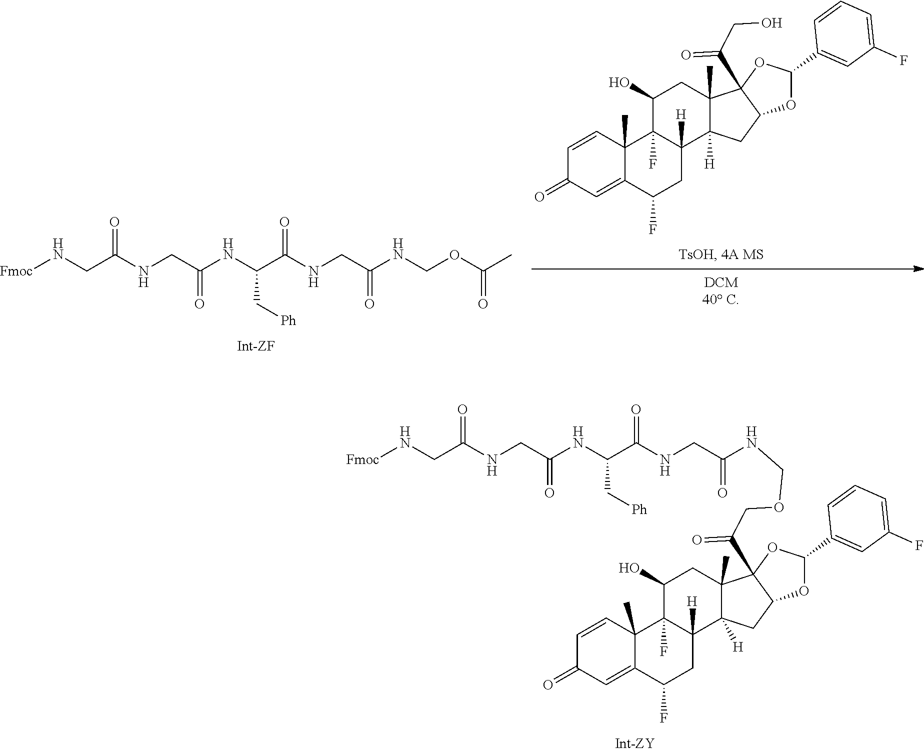

To a solution of Int-ZF (870.0 mg, 1.38 mmol, 3.0 equiv.) in dichloromethane (8.0 mL) was added starting material (238.8 mg, 0.46 mmol, 1.0 equiv.) at 25° C. under nitrogen atmosphere followed by the addition of p-toluenesulfonic acid (31.7 mg, 0.18 mmol, 0.4 equiv.) at 25° C. The resulting mixture was stirred for 12 h at 40° C. The mixture was cooled down to 25° C., diluted with water (300 mL), extracted dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 150.0 mg (30%) of Int-ZY was obtained as white solid. MS m/z [M+H]⁺ (ESI): 1088.42.

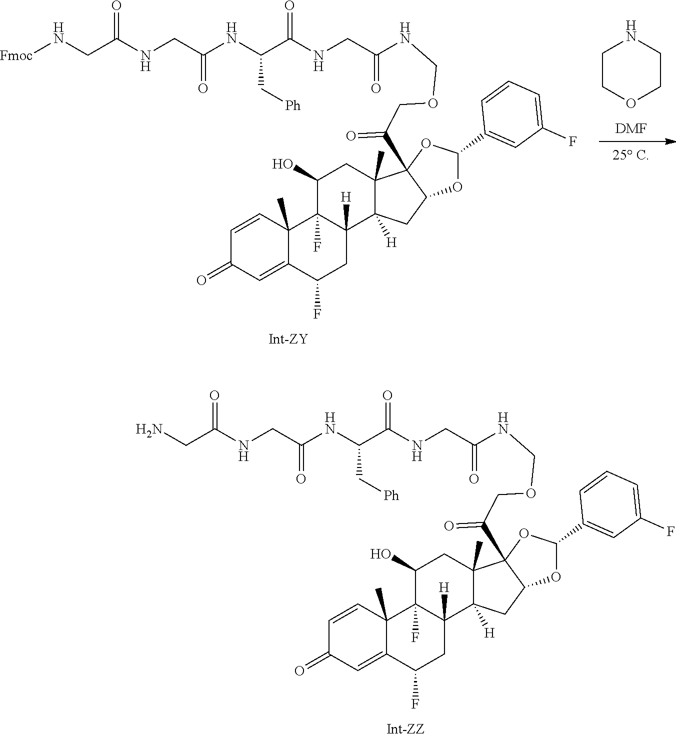

A solution of Int-ZY (150.0 mg, 0.14 mmol, 1.0 equiv.) in morpholine/N,N-dimethylformamide (0.1 mL/0.7 mL) with an inert atmosphere of nitrogen, the resulting solution was stirred for 1 h at 25° C., diluted with water (10 mL). The resulting mixture was extracted with dichloromethane (3×30 mL) and organic layers were washed with saturated sodium chloride solution (3×30 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 100.6 mg (84%) of Int-ZZ was obtained as white solid. MS m/z [M+H]⁺ (ESI): 866.35.

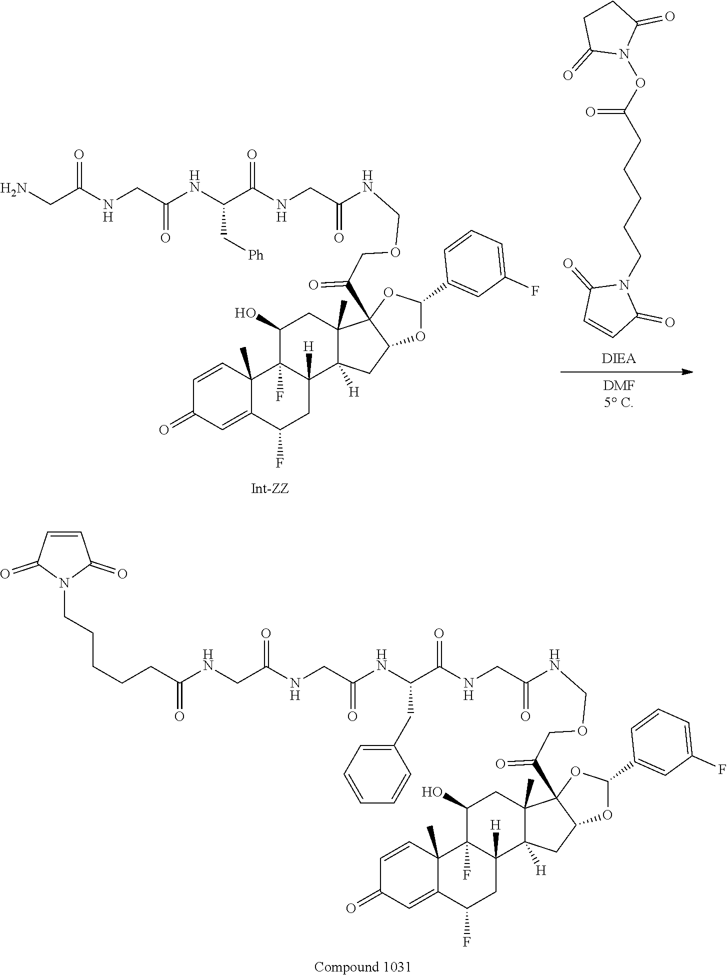

To a stirred mixture of Int-ZZ (50.0 mg, 0.06 mmol, 1.0 equiv.) in N,N-dimethylformamide (1.0 mL) with an inert atmosphere of nitrogen, was added N,N-diisopropylethylamine (22.4 mg, 0.17 mmol, 3.0 equiv.) and 2,5-dioxopyrrolidin-1-yl 6-(2,5-dioxopyrrol-1-yl)hexanoate (26.7 mg, 0.09 mmol, 1.5 equiv.) at 0° C. The resulting mixture was stirred for 3 h at 25° C. The crude product was purified by Prep-HPLC with the following conditions (IntelFlash-1): Column: XBridge Shield RP18 OBD Column, 30×150 mm, 5 μm; Mobile Phase A: water, Mobile Phase B: acetonitrile; Flow rate: 60 mL/min; Gradient: 32% B to 62% B in 9 min, 62% B; Wave Length: 254 nm. The resulting mixture was lyophilized in a cool and dry place. 5.1 mg (8%) of desired product was obtained as white solid. MS m/z [M+H]⁺ (ESI): 1059.40; 1H NMR (300 MHz, Methanol-d₄) δ: 1.00 (s, 3H), 1.29-1.32 (m, 2H), 1.54-1.65 (m, 9H), 1.79-1.83 (m, 3H), 2.23-2.28 (m, 6H), 3.00-3.29 (m, 2H), 3.44-3.49 (m, 2H), 3.76-3.86 (m, 6H), 4.37-4.43 (m, 3H), 4.67-4.77 (m, 3H), 5.04-5.06 (m, 1H), 5.50-5.61 (m, 1H), 6.25-6.31 (m, 2H), 6.77 (s, 2H), 7.19-7.38 (m, 10H).

### Example 71. Preparation of GR Agonist-Linker Compounds

The compounds in Table 10 below were prepared in a manner similar to that described Example 70 using the appropriate compounds as starting material.

TABLE 10
GR Agonist-Linker Compounds

| Structure | MS/¹H NMR |
|---|---|
| 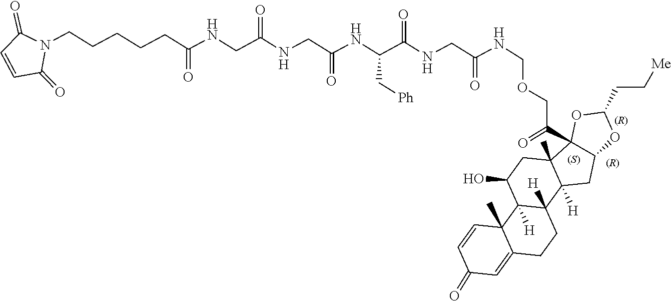 | MS m/z [M − H]⁻ (ESI): 969.35; ¹H NMR (400 MHZ, Methanol- d₄) δ: 0.79-0.91 (m, 7H), 1.22- 1.38 (m, 8H), 1.48 (s, 3H), 1.50-1.62 (m, 8H), 1.76-1.78 (m, 2H), 1.95-2.05 (m, 2H), 2.15-2.19 (m, 4H), 2.50-2.55 (m, 1H), 2.90-2.95 (m, 1H), 3.20-3.21 (m, 1H), 3.38-3.40 (m, 2H), 3.68-3.78 (m, 5H), 4.23-4.28 (m, 1H), 4.42-4.53 (m, 3H), 4.64-4.76 (m, 1H), 5.90 (s, 1H), 6.10-6.11 (m, 1H), 6.69 (s, 2H), 7.15-7.17 (m, 5H), 7.19-7.28 (m, 1H), 8.20-8.30 (m, 1H). |
| 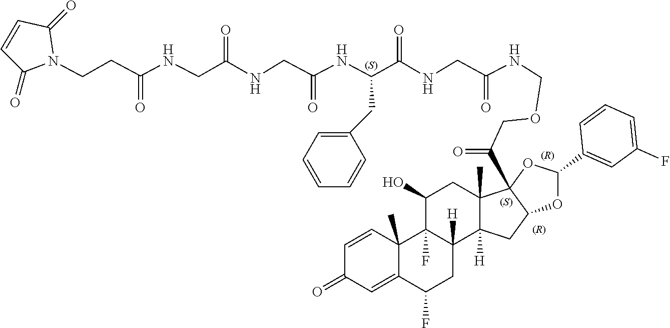 | MS m/z [M − H]⁻ (ESI): 1015.40; ¹H NMR (300 MHz, Methanol- d₄) δ: 0.99 (s, 3H), 1.50-1.57 (m, 4H), 1.78-1.82 (m, 3H), 2.20-2.35 (m, 3H), 2.52-2.56 (m, 2H), 2.60-2.70 (m, 1H), 3.00-3.03 (m, 1H), 3.10-3.18 (m, 1H), 3.76-3.92 (m, 8H), 4.37-4.44 (m, 3H), 4.68-4.80 (m, 2H), 5.04-5.06 (m, 1H), 5.50-5.61 (m, 2H), 6.26-6.30 (m, 2H), 6.78 (s, 2H), 7.19- 7.22 (m, 1H), 7.24-7.39 (m, 9H), 8.30-8.40 (m, 2H). |
| 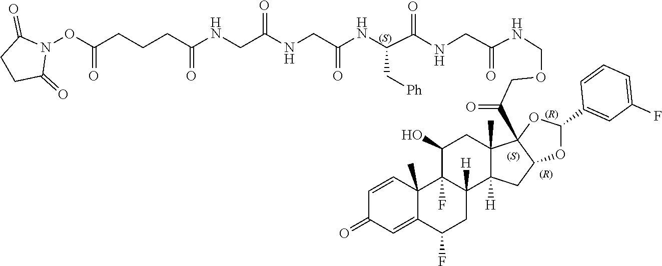 | MS m/z [M + H]⁺ (ESI): 1077.30; ¹H NMR (400 MHZ, Methanol-d₄) δ: 1.00 (s, 3H), 1.31 (s, 1H), 1.51-1.71 (m, 4H), 1.72-1.89 (m, 3H), 1.92- 2.09 (m, 2H), 2.15-2.25 (m, 1H), 2.26-2.44 (m, 4H), 2.61- 2.76 (m, 3H), 2.77-2.89 (m, 5H), 2.91-3.02 (m, 1H), 3.11- 3.23 (m, 1H), 3.69-3.98 (m, 6H), 4.24-4.38 (m, 1H), 4.39- 4.54 (m, 1H), 4.64-4.88 (m, 3H), 5.01-5.11 (m, 1H), 5.42- 5.68 (m, 2H), 6.21-6.38 (m, 2H), 7.08-7.29 (m, 7H), 7.30- 7.42 (m, 2H), 8.29-8.46 (m, 1H). |
| 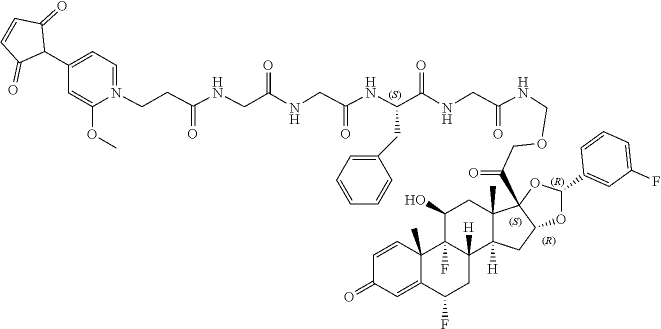 | MS m/z [M − H]⁻ (ESI): 1121.35; ¹H NMR (300 MHz, Methanol- d₄) δ: 0.98 (s, 3H), 1.48-1.62 (m, 4H), 1.72-1.88 (m, 3H), 2.12-2.27 (m, 1H), 2.28-2.39 (m, 2H), 2.46-2.79 (m, 3H), 2.81-2.92 (m, 2H), 2.93-3.03 (m, 1H), 3.14-3.26 (m, 1H), 3.69-3.99 (m, 9H), 4.21-4.54 (m, 3H), 4.64-4.82 (m, 3H), 4.99-5.09 (m, 1H), 5.38-5.70 (m, 2H), 6.19-6.36 (m, 3H), 6.43-6.59 (m, 1H), 6.89-7.02 (m, 1H), 7.03-7.16 (m, 2H), 7.17-7.42 (m, 10H). |

### Example 72. Preparation of 4-(5-(2,5-dioxo-2,5-dihydro-1H-pyrrol-1-yl)-2-(3-oxo-3-(2,3,5,6-tetrafluorophenoxy)propyl)phenoxy)butanoic acid (Int XX)

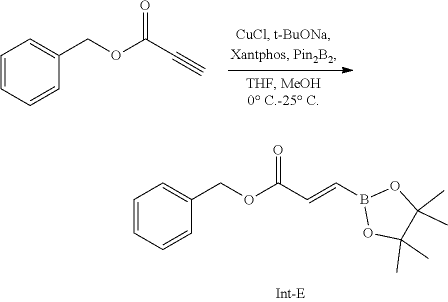 [SMILES: C#CC(=O)OCc1ccccc1 ; CC1(C)OB(/C=C/C(=O)OCc2ccccc2)OC1(C)C]

To a stirred solution of copper(I) chloride (0.09 g, 0.93 mmol, 0.03 equiv.) and 4,5-bis(diphenylphosphino)-9,9-dimethylxanthene (0.54 g, 0.93 mmol, 0.03 equiv.) in tetrahydrofuran (50 mL) was added sodium tert-butoxide (0.18 g, 1.87 mmol, 0.06 equiv.) at 0° C. The reaction solution was stirred at 0° C. for 1 h. To the above mixture was added 4,4,5,5-tetramethyl-2-(4,4,5,5-tetramethyl-1,3,2-dioxaborolan-2-yl)-1,3,2-dioxaborolane (15.85 g, 62.43 mmol, 2.0 equiv.) in portions at room temperature. The resulting mixture was stirred for additional 1 h at room temperature. To the above mixture was added benzyl prop-2-ynoate (5.0 g, 31.22 mmol, 1.0 equiv.) and methanol (1.5 g, 46.82 mmol, 1.5 equiv.) in portions at room temperature. The resulting mixture was stirred for additional 14 h at 25° C. The aqueous layer was extracted with methylene chloride (3×200 mL). The residue was purified by silica gel chromatography (column height: 250 mm, diameter: 100 mm, 100-200 mesh silica gel, petroleum ether/ethyl acetate=1/0, 3/1). 5.0 g (55%) of Int-E was obtained as a colorless liquid.

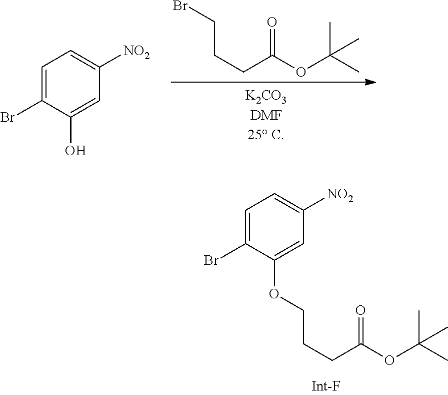 [SMILES: O=[N+]([O-])c1ccc(Br)c(O)c1 ; CC(C)(C)OC(=O)CCCBr ; CC(C)(C)OC(=O)CCCOc1cc([N+](=O)[O-])ccc1Br]

To a stirred solution of 2-bromo-5-nitrophenol (5.0 g, 22.9 mmol, 1.0 equiv.) and tert-butyl 4-bromobutanoate (7.7 g, 34.40 mmol, 1.5 equiv.) in N,N-dimethylformamide (100.0 mL) was added potassium carbonate (9.5 g, 68.8 mmol, 3 equiv.) in portions at 25° C. under nitrogen atmosphere. The resulting mixture was stirred for 3 h at 25° C. under nitrogen atmosphere, diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5 g (60%) of Int-F was obtained as a yellow solid.

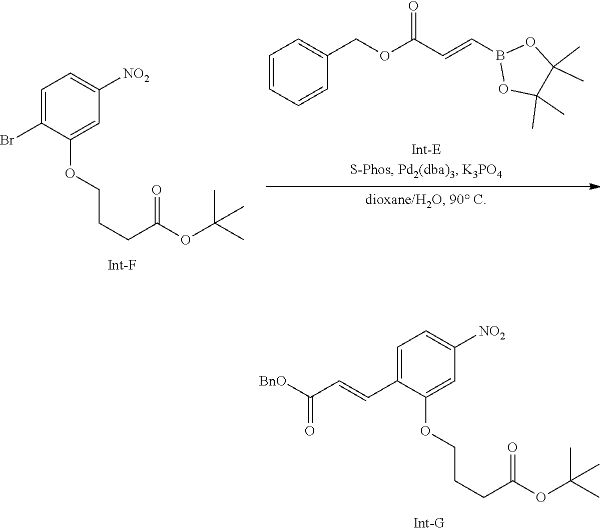 [SMILES: CC(C)(C)OC(=O)CCCOc1cc([N+](=O)[O-])ccc1Br ; CC1(C)OB(/C=C/C(=O)OCc2ccccc2)OC1(C)C ; CC(C)(C)OC(=O)CCCOc1cc([N+](=O)[O-])ccc1/C=C/C(=O)OCc1ccccc1]

To a stirred solution of Int-F (5.0 g, 13.88 mmol, 1.0 equiv.), benzyl (2E)-3-(4,4,5,5-tetramethyl-1,3,2-dioxaborolan-2-yl)prop-2-enoate Int-E (4.8 g, 16.65 mmol, 1.2 equiv.) and potassium phosphate tribasic (5.89 g, 27.76 mmol, 2.0 equiv.) in N,N-dimethylformamide (50.0 mL) were added 2-dicyclohexylphosphino-2′,6′-dimethoxy-1,1′-biphenyl (0.28 g, 0.69 mmol, 0.05 equiv.) and tris(dibenzylideneacetone)dipalladium (0.64 g, 0.69 mmol, 0.05 equiv.) in portions at room temperature under nitrogen atmosphere. And the reaction solution was stirred at 90° C. for another 12 hours under nitrogen atmosphere. The resulting mixture was cooled to 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3 g (49%) of Int-G was obtained as a yellow oil.

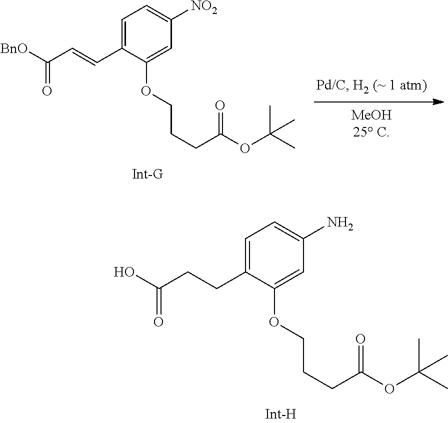 [SMILES: CC(C)(C)OC(=O)CCCOc1cc([N+](=O)[O-])ccc1/C=C/C(=O)OCc1ccccc1 ; CC(C)(C)OC(=O)CCCOc1cc(N)ccc1CCC(=O)O]

To a solution of Int-G (3.0 g, 6.80 mmol, 1.0 equiv.) in methanol (30.0 mL), was added palladium/carbon (1.0 g). The flask was evacuated and flushed five times with hydrogen. The resulting solution was stirred for 3 h at 25° C. The solids were filtered out and washed with methanol (3×10 mL). The resulting mixture was concentrated under reduced pressure and the crude product without further purification was used at next step directly. 2.5 g (81%) of Int-H was obtained as a white solid.

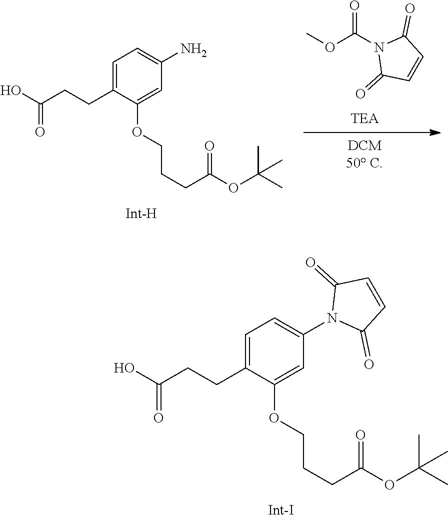 [SMILES: CC(C)(C)OC(=O)CCCOc1cc(N)ccc1CCC(=O)O ; COC(=O)N1C(=O)C=CC1=O ; CC(C)(C)OC(=O)CCCOc1cc(N2C(=O)C=CC2=O)ccc1CCC(=O)O]

To a solution of Int-H (2.5 g, 7.73 mmol, 1.0 equiv.) and methyl 2,5-dioxopyrrole-1-carboxylate (1.80 g, 11.59 mmol, 1.5 equiv.) in dichloromethane (25.0 mL) was added trimethylamine (1.56 g, 15.46 mmol, 2.0 equiv.) at room temperature under nitrogen atmosphere.

The resulting mixture was stirred for 4 h at 50° C. The resulting mixture was cooled to 25° C. and concentrated under reduced pressure. 1.5 g (48%) of Int-I was obtained as a yellow oil. MS m/z [M−H]⁻ (ESI): 402.16.

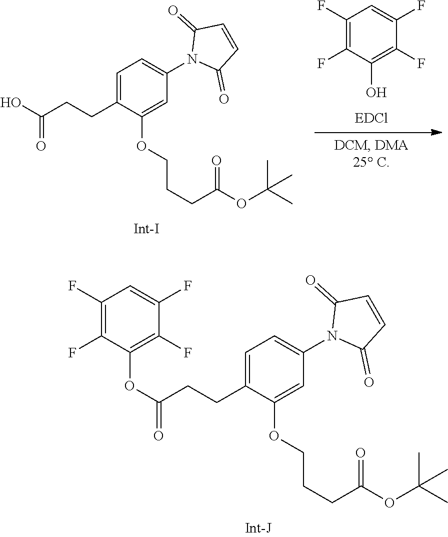 [SMILES: CC(C)(C)OC(=O)CCCOc1cc(N2C(=O)C=CC2=O)ccc1CCC(=O)O ; Oc1c(F)c(F)cc(F)c1F ; CC(C)(C)OC(=O)CCCOc1cc(N2C(=O)C=CC2=O)ccc1CCC(=O)Oc1c(F)c(F)cc(F)c1F]

A solution of Int-I (1.5 g, 3.71 mmol, 1 equiv.) in N,N-dimethylformamide (15 mL) was treated with 2,3,5,6-tetrafluor-phenol (1.85 g, 11.15 mmol, 3 equiv.) at room temperature under nitrogen atmosphere followed by the addition of 1-ethyl-3(3-dimethylpropylamine) carbodiimide (2.14 g, 11.15 mmol, 3 equiv.) at 25° C. The resulting mixture was stirred for 2 h at 25° C. under nitrogen atmosphere. The residue was purified by reversed-phase flash chromatography with the following conditions: column, C₁₈ silica gel; mobile phase, acetonitrile in water (0.1% formic acid), 10% to 100% gradient in 10 min; detector, UV 254 nm. The resulting mixture was concentrated under reduced pressure. 800 mg (39%) of Int-J was obtained as a colorless oil.

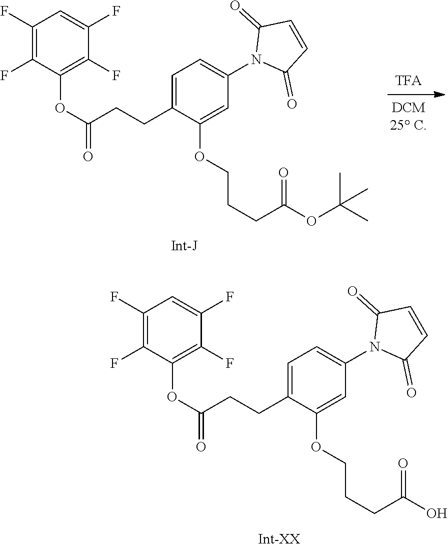 [SMILES: CC(C)(C)OC(=O)CCCOc1cc(N2C(=O)C=CC2=O)ccc1CCC(=O)Oc1c(F)c(F)cc(F)c1F ; O=C(O)CCCOc1cc(N2C(=O)C=CC2=O)ccc1CCC(=O)Oc1c(F)c(F)cc(F)c1F]

A solution of Int-J (800 mg, 1.45 mmol, 1 equiv.) in dichloromethane (6.0 mL) with an inert atmosphere of nitrogen, was added trifluoroacetic acid (2.0 mL) at 25° C. The resulting mixture was stirred for 2 h at 25° C. The resulting mixture was concentrated under reduced pressure. 700 mg (97%) of Int-XX was obtained as a yellow oil.

### Example 73. Preparation of (1-(2-(5-(2,5-dioxo-2,5-dihydro-1H-pyrrol-1-yl)-2-(3-oxo-3-(2,3,5,6-tetrafluorophenoxy)propyl)benzamido)acetamido)-2-oxo-6,9,12,15,18,21,24,27,30,33,36,39-dodecaoxa-3-azadotetracontan-42-oyl)-L-glutamic acid (Int 1)

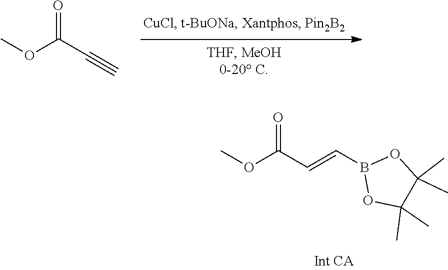

To a solution of copper(I) chloride (1.06 g, 10.7 mmol, 0.03 equiv.) and 4,5-Bis(diphenylphosphino)-9,9-dimethylxanthene (6.19 g, 10.7 mmol, 0.03 equiv.) in tetrahydrofuran (450.0 mL) was added sodium tert-butoxide (2.06 g, 21.4 mmol, 0.06 equiv.) at 0° C. The reaction solution was stirred at 0° C. for 1 hour, followed by addition of a solution of bis(pinacolato)diboron (90.6 g, 357 mmol, 1.0 equiv.) in tetrahydrofuran (150.0 mL). The reaction solution was stirred under nitrogen atmosphere at 20° C. for one hour. Methyl prop-2-ynoate (30.0 g, 357.0 mmol, 1.0 equiv.) and methanol (22.9 g, 714 mmol, 2.0 equiv.) were added to the above reaction solution. And the reaction solution was stirred at 20° C. for another 12 h. The result mixture was poured into ice-water (w/w=1/1) (300 mL) and stirred for 5 min. The aqueous phase was extracted with ethyl acetate (200 mL×3). The combined organic phase was washed with brine (50 mL×2), dried with anhydrous sodium sulfate, filtered, and concentrated in vacuum. The residue was purified by silica gel chromatography (column height: 250 mm, diameter: 100 mm, 100-200 mesh silica gel, petroleum ether/ethyl acetate=1/0, 3/1) to afford Int CA (70 g, 92%) as colorless oil: ¹H NMR (400 MHz, CDCl₃) δ 6.83-6.75 (m, 1H), 6.68-6.59 (m, 1H), 3.77 (s, 3H), 1.29 (s, 12H).

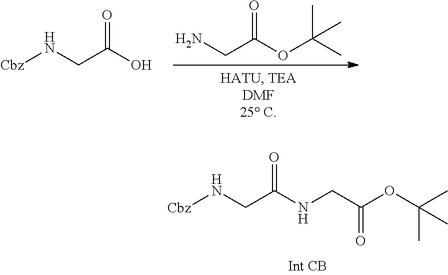

To a solution of 2-(benzyloxycarbonylamino) acetic acid (5.50 g, 26.3 mmol, 1.0 equiv.) in N,N-dimethylformamide (100.0 mL) was added triethylamine (7.98 g, 78.9 mmol, 3.0 equiv.), 2-(7-Azabenzotriazol-1-yl)-N,N,N′,N′-tetramethyluronium hexafluorophosphate (12.0 g, 31.5 mmol, 1.2 equiv.) and tert-butyl 2-aminoacetate (3.45 g, 26.3 mmol, 1.0 equiv.), and then stirred at 25° C. for 2 h. The reaction mixture was quenched by addition water (150 mL) at 0° C., and then extracted with ethyl acetate (100 mL×3). The combined organic layers were washed with water (80 mL×3), then were washed with brine (100 mL), dried over sodium sulfate, filtered, and concentrated under reduced pressure to give a residue. The residue was purified by silica gel chromatography (column height: 250 mm, diameter: 100 mm, 100-200 mesh silica gel, ethyl acetate/petroleum ether=1/1) to give Int CB (7.7 g, 91%) was obtained as a yellow oil: ¹H NMR (CDCl₃, 400 MHz) δ7.40-7.31 (m, 5H), 5.14 (s, 2H), 3.98-3.89 (m, 4H), 1.47 (s, 9H).

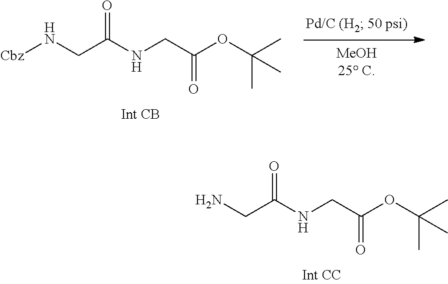 [SMILES: CC(C)(C)OC(=O)CNC(=O)CNC(=O)OCc1ccccc1 ; CC(C)(C)OC(=O)CNC(=O)CN]

To a solution of Int CB (8.8 g, 27.3 mmol, 1.0 equiv.) in methanol (150 mL) was added palladium/carbon (10%, 2.5 g) under nitrogen atmosphere. The suspension was degassed and purged with hydrogen for 3 times. The mixture was stirred under hydrogen (50 Psi) at 25° C. for 2 h. The reaction mixture was filtered and concentrated under reduced pressure to give a residue. Int CC (4.2 g, 81%) was obtained as a white solid. MS m/z [M+H]⁺ (ESI): 189.12; ¹H NMR (CDCl₃, 400 MHz) δ 7.67 (s, 1H), 3.96 (d, J=5.6 Hz, 2H), 3.39 (s, 2H), 1.75 (s, 2H), 1.46 (s, 9H).

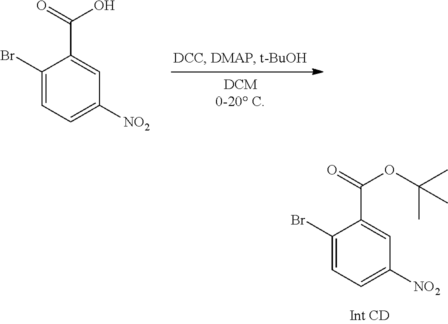 [SMILES: O=C(O)c1cc([N+](=O)[O-])ccc1Br ; CC(C)(C)OC(=O)c1cc([N+](=O)[O-])ccc1Br]

To a mixture of 2-bromo-5-nitro-benzoic acid (9.7 g, 39.4 mmol, 1.0 equiv.) in dichloromethane (100 mL) was added dicyclohexylcarbodiimide (8.95 g, 43.4 mmol, 1.1 equiv.) and 4-dimethylaminopyridine (2.41 g, 19.7 mmol, 0.5 equiv.) at 0° C. The mixture was stirred at 0° C. for 10 min, then tert-butanol (4.4 g, 59.1 mmol, 1.5 equiv.) was added and stirred at 20° C. for 12 h. The mixture was poured into ice-water (w/w=1/1) (50 mL) and stirred for 10 min. The aqueous phase was extracted with dichloromethane (500 mL×3). The combined organic phase was washed with brine (100 mL×3), dried with anhydrous sodium sulfate, filtered, and concentrated in vacuum. The residue was purified by silica gel chromatography (column height: 250 mm, diameter: 100 mm, 100-200 mesh silica gel, petroleum ether/ethyl acetate=1/0, 5/1) to afford Int CD (9.2 g, 77%) as white solid: ¹H NMR (400 MHz, CDCl₃) δ 8.52 (d, J=2.8 Hz, 1H), 8.13 (dd, J=2.8, 8.8 Hz, 1H), 7.83 (d, J=8.8 Hz, 1H), 1.65 (s, 9H).

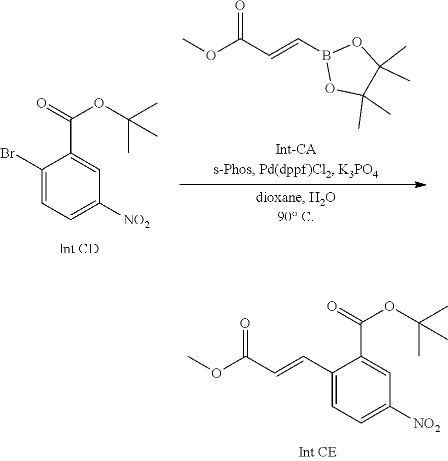

A mixture of Int CA (11.2 g, 53.0 mmol, 2.5 equiv.), Int CD (6.40 g, 21.2 mmol, 1.0 equiv.), potassium phosphate tribasic (6.74 g, 31.8 mmol, 1.5 equiv.), 2-dicyclohexylphosphino-2′,6′-dimethoxy-1,1′-biphenyl (870 mg, 2.12 mmol, 0.1 equiv.), tris(dibenzylideneacetone)dipalladium (970 mg, 1.06 mmol, 0.05 equiv.) in dioxane (120.0 mL) and water (25.0 mL) was degassed and purged with nitrogen for 3 times, and then the mixture was stirred at 90° C. for 12 h under nitrogen atmosphere. The mixture was poured into ice-water (w/w=1/1) (30 mL) and stirred for 10 min. The aqueous phase was extracted with ethyl acetate (500 mL×3). The combined organic phase was washed with brine (100 mL×3), dried with anhydrous sodium sulfate, filtered, and concentrated in vacuum. The residue was purified by silica gel chromatography (column height: 250 mm, diameter: 100 mm, 100-200 mesh silica gel, petroleum ether/ethyl acetate=1/0, 3/1) obtained Int CE (6 g, 92%) as yellow solid: ¹H NMR (400 MHz, CDCl₃) δ 8.74 (d, J=2.4 Hz, 1H), 8.41 (d, J=15.6 Hz, 1H), 8.34 (dd, J=2.4, 8.4 Hz, 1H), 7.72 (d, J=8.4 Hz, 1H), 6.38 (d, J=15.6 Hz, 1H), 3.85 (s, 3H), 1.65 (s, 9H).

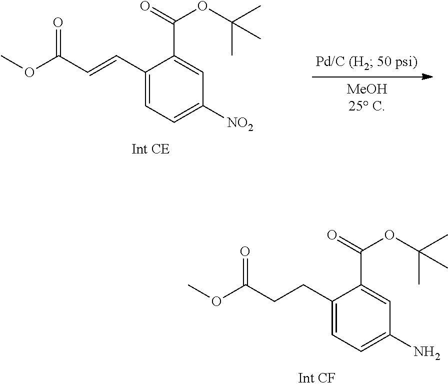 [SMILES: COC(=O)/C=C/c1ccc([N+](=O)[O-])cc1C(=O)OC(C)(C)C ; COC(=O)CCc1ccc(N)cc1C(=O)OC(C)(C)C]

A mixture of Int CE (4.0 g, 13.0 mmol, 1.0 equiv.), palladium/carbon (10%, 400.0 mg) in methanol (50.0 mL) was degassed and purged with hydrogen for 3 times, and then the mixture was stirred at 25° C. for 3 h under hydrogen atmosphere. The mixture was filtered and concentrated in vacuum to afford Int CF (3.50 g, 96%) as yellow oil: [M+H]⁺ (ESI): 280.30; ¹H NMR (400 MHz, CDCl₃) δ 7.14 (d, J=2.4 Hz, 1H), 7.04 (d, J=8.0 Hz, 1H), 6.73 (dd, J=2.4, 8.0 Hz, 1H), 3.67 (s, 3H), 3.12 (t, J=8.0 Hz, 2H), 2.61 (t, J=8.0 Hz, 2H), 1.59 (s, 9H).

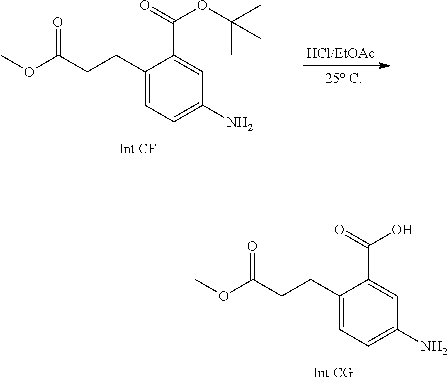

To a solution of Int CF (3.5 g, 12.5 mmol, 1.0 equiv.) in ethyl acetate (10.0 mL) was added hydrochloride (gas)/ethyl acetate (4 mol/L, 50 mL, 16.0 equiv.), and then stirred at 25° C. for 12 hours. The mixture was concentrated in vacuum to afford Int CG (3.20 g, 98%) as white solid: [M+H]⁺ (ESI): 224.23; ¹H NMR (400 MHz, DMSO-d₆) δ 7.69 (d, J=2.0 Hz, 1H), 7.39-7.31 (m, 2H), 3.57 (s, 3H), 3.15 (t, J=7.6 Hz, 2H), 2.59 (t, J=7.6 Hz, 2H).

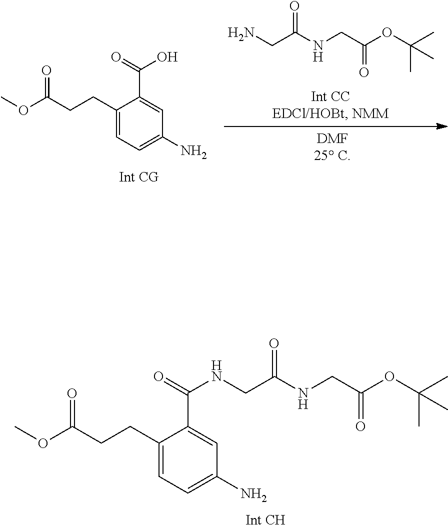 [SMILES: COC(=O)CCc1ccc(N)cc1C(=O)O ; CC(C)(C)OC(=O)CNC(=O)CN ; COC(=O)CCc1ccc(N)cc1C(=O)NCC(=O)NCC(=O)OC(C)(C)C]

To a solution of Int CG (3.20 g, 12.3 mmol, 1.0 equiv.) in N,N-dimethylformamide (40 mL) was added 4-methylmorpholine (3.74 g, 37.0 mmol, 3.0 equiv.), 1-hydroxybenzotriazole (833 mg, 6.16 mmol, 0.5 equiv.), N-(3-dimethylaminopropyl)-N′-ethylcarbodiimide hydrochloride (4.72 g, 24.7 mmol, 2.0 equiv.) and tert-butyl 2-[(2-aminoacetyl)amino]acetate (2.78 g, 14.8 mmol, 1.2 equiv.) at 0° C., and then stirred at 20° C. for 2 h. The mixture was poured into ice-water (w/w=1/1) (40 mL) and stirred for 10 min. The aqueous phase was extracted with ethyl acetate (500 mL×3). The combined organic phase was washed with brine (200 mL×3), dried with anhydrous sodium sulfate, filtered, and concentrated in vacuum. The residue was purified by silica gel chromatography (column height: 250 mm, diameter: 100 mm, 100-200 mesh silica gel, petroleum ether/ethyl acetate=1/0, 0/1) to afford Int CH (3.8 g, 78%) as yellow oil: [M+H]⁺ (ESI): 394.35; ¹H NMR (400 MHz, CDCl₃) δ 7.04 (d, J=8.0 Hz, 2H), 6.79-6.66 (m, 3H), 4.15 (d, J=5.6 Hz, 2H), 3.97 (d, J=5.2 Hz, 2H), 3.61 (s, 3H), 2.99 (t, J=7.2 Hz, 2H), 2.68 (t, J=7.2 Hz, 2H), 1.47 (s, 9H).

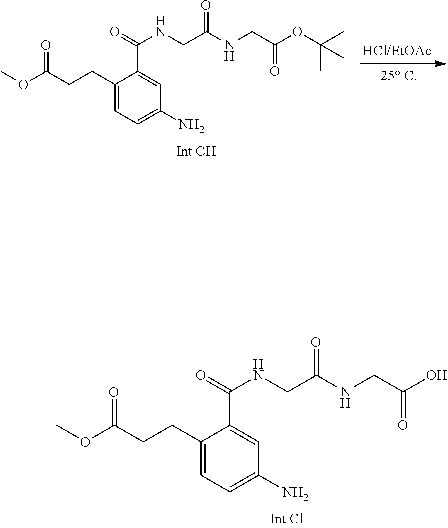

To a solution of Int CH (0.60 g, 1.53 mmol, 1.0 equiv.) in ethyl acetate (5.0 mL) was added hydrochloride (gas)/ethyl acetate (4 mol/L, 10 mL, 26.2 equiv.), and then stirred at 25° C. for 12 h. The mixture was concentrated in vacuum to afford Int CI (550 mg, 96% yield) as white solid: [M+H]⁺ (ESI): 338.14; ¹H NMR (400 MHz, DMSO-d₆) δ 8.57 (t, J=6.0 Hz, 1H), 8.25 (t, J=6.4 Hz, 1H), 7.28 (d, J=8.0 Hz, 1H), 7.20-7.10 (m, 2H), 3.89 (d, J=6.0 Hz, 2H), 3.81 (d, J=6.0 Hz, 2H), 3.57 (s, 3H), 2.90 (t, J=8.0 Hz, 2H), 2.60 (t, J=8.0 Hz, 2H).

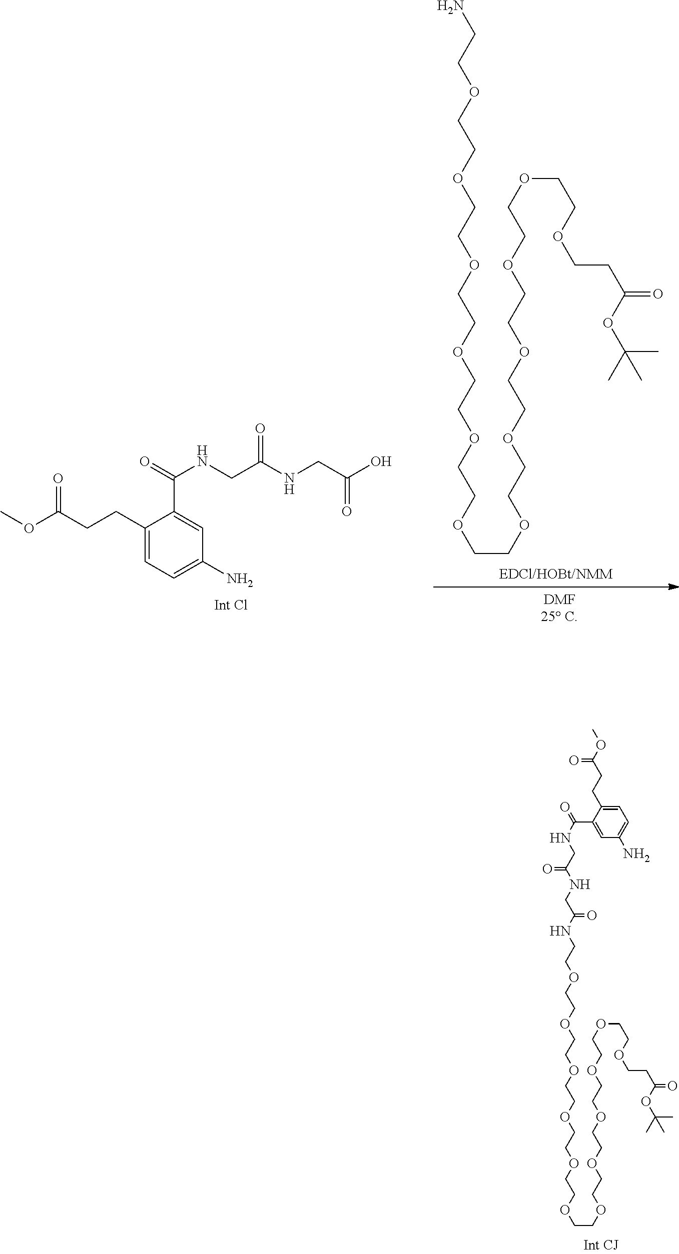 [SMILES: COC(=O)CCc1ccc(N)cc1C(=O)NCC(=O)NCC(=O)O ; CC(C)(C)OC(=O)CCOCCOCCOCCOCCOCCOCCOCCOCCOCCOCCOCCOCCN ; COC(=O)CCc1ccc(N)cc1C(=O)NCC(=O)NCC(=O)NCCOCCOCCOCCOCCOCCOCCOCCOCCOCCOCCOCCOCCC(=O)OC(C)(C)C]

A mixture of Int CI (320.0 mg, 0.86 mmol, 1.0 equiv.), tert-butyl 3-[2-[2-[2-[2-[2-[2 [2-[2-[2-[2-[2-(2-aminoethoxy)ethoxy]ethoxy]ethoxy]ethoxy]ethoxy]ethoxy]-ethoxy]ethoxy]ethoxy]ethoxy]ethoxy]propanoate (576.0 mg, 0.86 mmol, 1.0 equiv.), N-(3-dimethylaminopropyl)-N′-ethylcarbodiimide hydrochloride (328.0 mg, 1.71 mmol, 2.0 equiv.), 1-hydroxybenzotriazole (57.8 mg, 0.43 mmol, 0.5 equiv.) and 4-methylmorpholine (259.0 mg, 2.57 mmol, 3.0 equiv.) in N,N-dimethylformamide (5.0 mL) was stirred at 25° C. for 2 hours. The reaction mixture was quenched by addition water (10 mL) at 0° C., and then extracted with methylene chloride/isopropyl alcohol (v/v=3:1, 100 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 950 mg (90%) of Int CJ was obtained as yellow oil. [M+H]⁺ (ESI): 994.10.

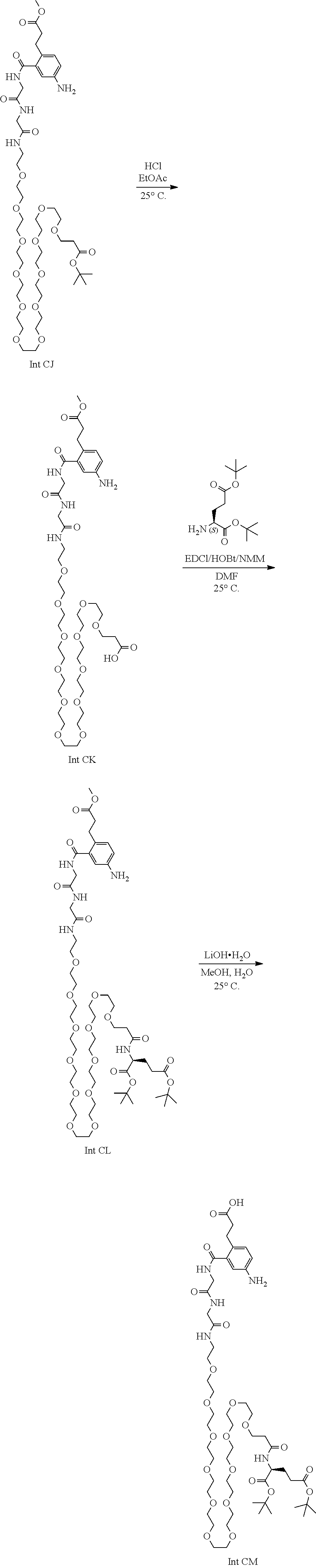

To a solution of Int CJ (850 mg, 956 μmol, 1.0 equiv.) in dioxane (5.0 mL) was added hydrochloride (gas)/ethyl acetate (15 mL, 4 mol/L). The mixture was stirred at 25° C. for 2 hours. The reaction mixture was concentrated under reduced pressure. Int CK (800 mg, crude) was obtained as a yellow solid. [M+H]⁺ (ESI): 936.20.

A mixture of Int CK (800 mg, 0.903 mmol, 1.0 equiv.), ditert-butyl (2S)-2-aminopentanedioate (588 mg, 1.99 mmol, 2.2 equiv., HCl), 1-ethyl-3(3-dimethylpropylamine) carbodiimide (346 mg, 1.81 mmol, 2.0 equiv.), 1-hydroxybenzotriazole (61.0 mg, 0.451 mmol, 0.5 equiv.) and 4-methylmorpholine (274 mg, 2.71 mmol, 3.0 equiv.) in N,N-dimethylformamide (10.0 mL) was stirred at 25° C. for 2 hours. The reaction mixture was quenched by addition water (20 mL) at 0° C., and then extracted with methylene chloride/isopropyl alcohol (v:v=3:1, 50 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 900 mg (89%) of Int CL was obtained as yellow oil. [M+H]⁺ (ESI): 1179.05.

To a solution of Int CL (800 mg, 0.68 mmol, 1.0 equiv.) in methanol (8.0 mL) and water (2.5 mL) was added sodium hydroxide (136 mg, 3.39 mmol, 5.0 equiv.), and then stirred at 0° C. for 2 h. The mixture was concentrated in vacuum to remove methanol, and then diluted with water (20 mL), the pH of the aqueous phase was adjusted to ˜7 with hydrochloric acid (2 mol/L), and extracted with methylene chloride/isopropyl alcohol (v/v=3:1, 30 mL×5). The combined organic layers were dried over sodium sulfate, filtered, and concentrated under reduced pressure to give a residue to afford Int CM (700 mg, crude) as red oil. [M+H]⁺ (ESI): 1165.10.

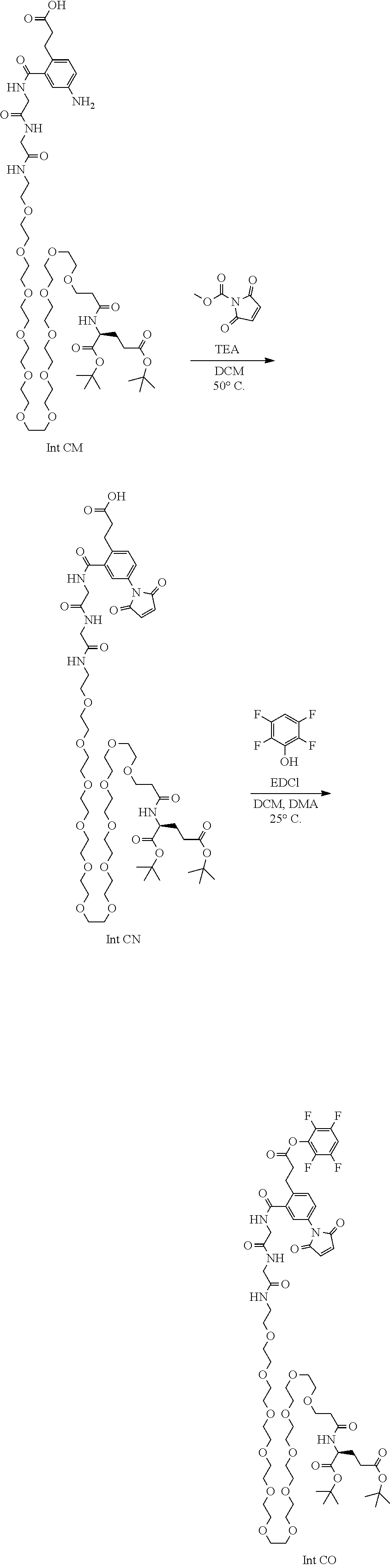 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N)ccc1CCC(=O)O)C(=O)OC(C)(C)C ; COC(=O)N1C(=O)C=CC1=O ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)O)C(=O)OC(C)(C)C ; Oc1c(F)c(F)cc(F)c1F ; CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)OC(C)(C)C]

To a solution of Int CM (700 mg, 0.60 mmol, 1.0 equiv.) and methyl 2,5-dioxopyrrole-1-carboxylate (139.8 mg, 0.92 mmol, 1.5 equiv.) in dichloromethane (7.0 mL) was added trimethylamine (182.2 mg, 1.80 mmol, 3.0 equiv.), and then stirred at 50° C. for 4 h. The pH of the mixture was adjusted ˜6 with trifluoroacetic acid, and diluted with addition water (10 mL) and extracted with methylene chloride: isopropyl alcohol (v/v=3:1, 30 mL×5). The combined organic layers were concentrated under reduced pressure to give a residue. Column, C₁₈ silica gel; mobile phase, acetonitrile in water (0.1% trifluoroacetic acid/formic acid), 10% to 70% gradient in 20 min; detector, UV 254 nm. 306 mg (41%) of Int CN was obtained as colorless oil. [M+H]⁺ (ESI): 1245.32.

A mixture of 2,3,5,6-tetrafluorophenol (108 mg, 0.65 mmol, 3.0 equiv.), Int CN (270 mg, 216 μmol, 1.0 equiv.) and 1-ethyl-3(3-dimethylpropylamine) carbodiimide (166 mg, 867 μmol, 4.0 equiv.) in N,N-dimethylformamide (3.0 mL) was stirred at 25° C. for 2 h. The reaction mixture was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 90 mg (30%) of Int CO was obtained as colorless oil. [M+H]⁺ (ESI): 1394.45; ¹H NMR (DMSO-d₆, 400 MHz) δ 7.94-7.84 (m, 1H), 7.50 (d, J=8.4 Hz, 1H), 7.43-7.34 (m, 2H), 7.18 (s, 2H), 4.13 (dd, J=5.2, 9.1 Hz, 1H), 3.89 (s, 2H), 3.70 (s, 2H), 3.60-3.58 (m, 3H), 3.51-3.47 (m, 44H), 3.39 (t, J=6.0 Hz, 2H), 3.22-3.17 (m, 2H), 3.14 (s, 3H), 2.42-2.32 (m, 2H), 2.27-2.19 (m, 2H), 1.95-1.83 (m, 1H), 1.77-1.65 (m, 1H), 1.38 (s, 18H).

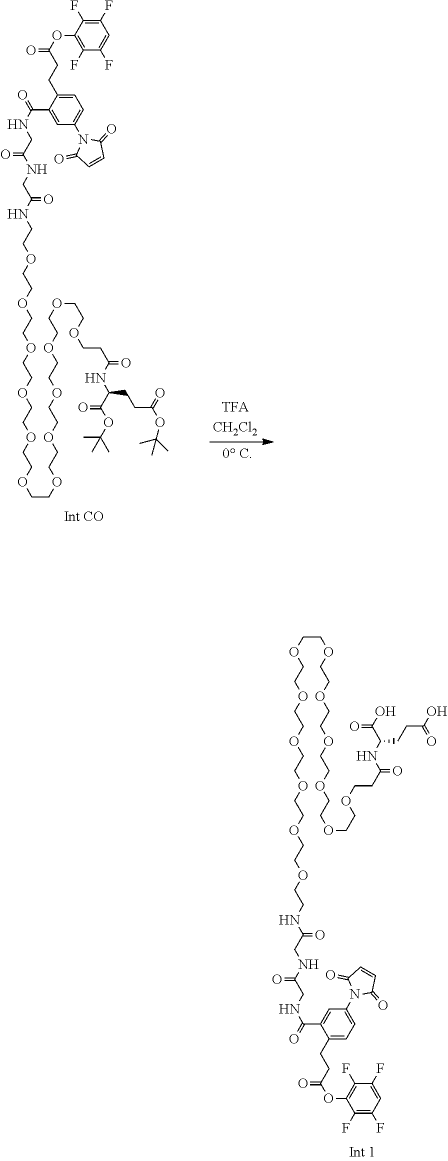 [SMILES: CC(C)(C)OC(=O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)OC(C)(C)C ; O=C(O)CC[C@H](NC(=O)CCOCCOCCOCCOCCOCCOCCOCCOCCOCCOCCOCCOCCNC(=O)CNC(=O)CNC(=O)c1cc(N2C(=O)C=CC2=O)ccc1CCC(=O)Oc1c(F)c(F)cc(F)c1F)C(=O)O]

A solution of Int CO (90 mg, 0.07 mmol, 1.0 equiv.) in dichloromethane (1.0 mL) with an inert atmosphere of nitrogen, was added trifluoroacetic acid (0.25 mL) at 0° C. The resulting solution was stirred for 4 h at 0° C. and concentrated under vacuum and concentrated under reduced pressure. 90 mg (crude) of Int 1 was obtained as a yellow oil. [M+H]⁺ (ESI): 1279.95.

### Example 74. Preparation of (1-(2-(2-(3-(((S)-1-(((S)-1-((4-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenyl)amino)-1-oxopropan-2-yl)amino)-3-methyl-1-oxobutan-2-yl)amino)-3-oxopropyl)-5-(2,5-dioxo-2,5-dihydro-1H-pyrrol-1-yl)benzamido)acetamido)-2-oxo-6,9,12,15,18,21,24,27,30,33,36,39-dodecaoxa-3-azadotetracontan-42-oyl)-L-glutamic acid

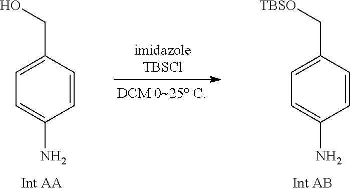 [SMILES: Nc1ccc(CO)cc1 ; CC(C)(C)[Si](C)(C)OCc1ccc(N)cc1]

To a solution of (4-aminophenyl)methanol, Int-AA, (5.00 g, 40.6 mmol, 1.0 eq) in DCM (50 mL) was added imidazole (4.15 g, 60.9 mmol, 1.5 eq) and TBSCl (7.34 g, 48.7 mmol, 5.97 mL, 1.2 eq) at 0° C., and then stirred at 25° C. for 1 hr. The reaction mixture was quenched by addition H₂O 100 mL at 0° C., and then extracted with DCM (30 mL×3). The combined organic layers were washed with brine 50 mL, dried over Na₂SO₄, filtered, and concentrated under reduced pressure to give a residue. The residue was purified by flash silica gel chromatography (Biotage®; 80 g SepaFlash® Silica Flash Column, Eluent of 50˜70% Ethyl acetate/petroleum ether gradient @ 80 mL/min). 4-[[tert-butyl(dimethyl)silyl]oxymethyl]aniline, Int-AB, (9.6 g, 40.44 mmol, 99.60% yield) was obtained as colorless oil: ¹H NMR (400 MHz, CDCl₃) δ 7.13 (d, J=8.0 Hz, 2H), 6.67 (d, J=8.0 Hz, 2H), 4.64 (s, 2H), 0.94 (s, 9H), 0.10 (s, 6H); LC/MS [M+H]238.2 (calculated); LC/MS [M+H] 238.1 (observed).

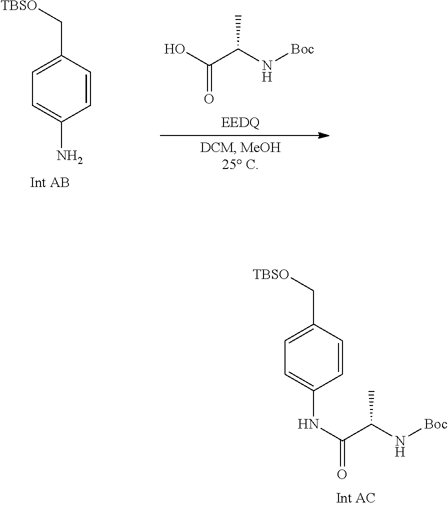 [SMILES: CC(C)(C)[Si](C)(C)OCc1ccc(N)cc1 ; C[C@H](NC(=O)OC(C)(C)C)C(=O)O ; C[C@H](NC(=O)OC(C)(C)C)C(=O)Nc1ccc(CO[Si](C)(C)C(C)(C)C)cc1]

To a solution of 4-[[tert-butyl(dimethyl)silyl]oxymethyl]aniline, Int-AB, (9.34 g, 39.3 mmol, 1 eq) and (2S)-2-(tert-butoxycarbonylamino)propanoic acid (8.93 g, 47.2 mmol, 1.2 eq) in DCM (50 mL) and MeOH (50 mL) was added EEDQ (29.2 g, 118 mmol, 3.0 eq), and then stirred at 25° C. for 1 hr. The reaction mixture was quenched by addition H₂O 100 mL at 0° C., then extracted with EtOAc (50 mL×3). The combined organic layers were washed with brine 50 mL, dried over Na₂SO₄, filtered, and concentrated under reduced pressure to give a residue. The residue was purified by flash silica gel chromatography (Biotage®; 120 g SepaFlash® Silica Flash Column, Eluent of 15-30% Ethyl acetate/petroleum ether gradient @ 80 mL/min). tert-butyl N-[(1S)-2-[4-[[tert-butyl(dimethyl)silyl]oxymethyl]anilino]-1-methyl-2-oxo-ethyl]carbamate, Int-AC, (25 g, crude) was obtained as oil.

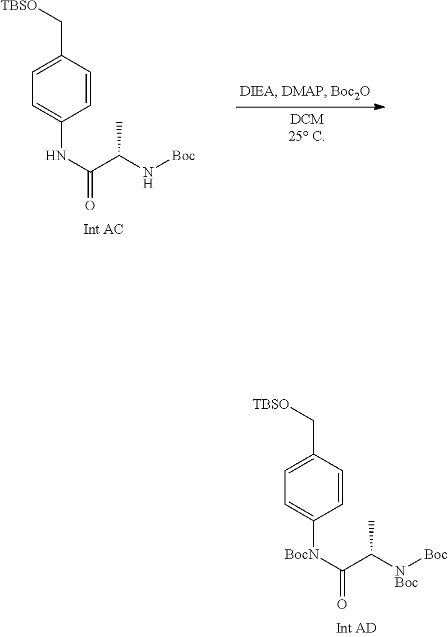 [SMILES: C[C@H](NC(=O)OC(C)(C)C)C(=O)Nc1ccc(CO[Si](C)(C)C(C)(C)C)cc1 ; C[C@@H](C(=O)N(C(=O)OC(C)(C)C)c1ccc(CO[Si](C)(C)C(C)(C)C)cc1)N(C(=O)OC(C)(C)C)C(=O)OC(C)(C)C]

To a solution of tert-butyl N-[(1S)-2-[4-[[tert-butyl(dimethyl)silyl]oxymethyl]anilino]-1-methyl-2-oxo-ethyl]carbamate, Int-AC, (10.0 g, 24.5 mmol, 1.0 eq) in DCM (100 mL) was added DMAP (2.99 g, 24.5 mmol, 1.0 eq), DIEA (9.49 g, 73.4 mmol, 12.8 mL, 3.0 eq) and Boc₂O (16.0 g, 73.4 mmol, 16.9 mL, 3.0 eq). The mixture was stirred at 40° C. for 24 hrs. The reaction mixture was quenched by addition H₂O 100 mL at 0° C., and then extracted with DCM (50 mL×2). The combined organic layers were washed with brine 100 mL, dried over Na₂SO₄, filtered, and concentrated under reduced pressure to give a residue. The residue was purified by flash silica gel chromatography (Biotage®; 120 g SepaFlash® Silica Flash Column, Eluent of 10-20% Ethyl acetate/petroleum ether gradient @ 80 mL/min). tert-butyl N-[(2S)-2-[bis(tert-butoxycarbonyl)amino]propanoyl]-N-[4-[[tert-buty l(dimethyl)silyl]oxymethyl]phenyl]carbamate, Int-AD, (4.70 g, 7.72 mmol, 31.54% yield) was obtained as colorless oil: ¹H NMR (CDCl₃, 400 MHz) δ 7.31 (d, J=8.4 Hz, 2H), 7.11 (d, J=8.4 Hz, 2H), 5.48-5.43 (m, 1H), 4.75 (s, 2H), 1.55-1.50 (m, 27H), 1.02 (d, J=6.4 Hz, 3H), 0.94 (s, 9H), 0.08 (s, 6H); LC/MS [M+Na] 631.35 (calculated); LC/MS [M+Na] 631.3 (observed).

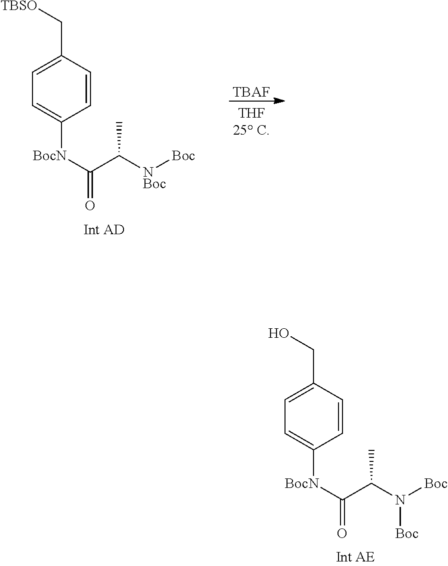 [SMILES: C[C@@H](C(=O)N(C(=O)OC(C)(C)C)c1ccc(CO[Si](C)(C)C(C)(C)C)cc1)N(C(=O)OC(C)(C)C)C(=O)OC(C)(C)C ; "TBAF" ; C[C@@H](C(=O)N(C(=O)OC(C)(C)C)c1ccc(CO)cc1)N(C(=O)OC(C)(C)C)C(=O)OC(C)(C)C]

To a solution of tert-butyl N-[(2S)-2-[bis(tert-butoxycarbonyl)amino]propanoyl]-N-[4-[[tert-butyl(dimethyl)silyl]oxymethyl]phenyl]carbamate, Int-AD, (4.68 g, 7.69 mmol, 1.0 eq) in THF (50 mL) was added TBAF (1 M, 15.4 mL, 2.0 eq), and then stirred at 25° C. for 1 hr. The reaction mixture was quenched by addition H₂O 100 mL at 0° C., then extracted with EtOAc (50 mL×3). The combined organic layers were washed with brine 50 mL, dried over Na₂SO₄, filtered, and concentrated under reduced pressure to give a residue. The residue was purified by flash silica gel chromatography (Biotage®; 40 g SepaFlash® Silica Flash Column, Eluent of 50-70% Ethyl acetate/petroleum ether gradient @ 60 mL/min). tert-butyl N-[(2S)-2-[bis(tert-butoxycarbonyl)amino]propanoyl]-N-[4-(hydroxy methyl)phenyl]carbamate, Int-AE, (2.10 g, 4.25 mmol, 55.24% yield) was obtained as colorless oil: ¹H NMR (400 MHz, CDCl₃) δ 7.36 (d, J=8.4 Hz, 2H), 7.15 (d, J=8.4 Hz, 2H), 5.49-5.44 (m, 1H), 4.70 (d, J=2.8 Hz, 2H), 1.53 (s, 21H), 1.35 (s, 9H), LC/MS [M+H] 517.2 (calculated), LC/MS [M+H] 517.2 (observed).

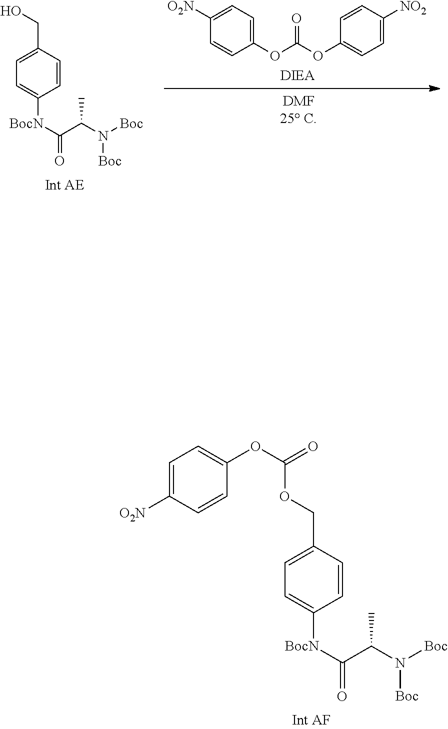 [SMILES: C[C@@H](C(=O)N(C(=O)OC(C)(C)C)c1ccc(CO)cc1)N(C(=O)OC(C)(C)C)C(=O)OC(C)(C)C ; O=C(Oc1ccc([N+](=O)[O-])cc1)Oc1ccc([N+](=O)[O-])cc1 ; C[C@@H](C(=O)N(C(=O)OC(C)(C)C)c1ccc(COC(=O)Oc2ccc([N+](=O)[O-])cc2)cc1)N(C(=O)OC(C)(C)C)C(=O)OC(C)(C)C]

To a solution of tert-butyl N-[(2S)-2-[bis(tert-butoxycarbonyl)amino]propanoyl]-N-[4-(hydroxymethyl)phenyl]carbamate, Int-AE, (1.00 g, 2.02 mmol, 1.0 eq) in DCM (15 mL) was added DIEA (784 mg, 6.07 mmol, 1.06 mL, 3.0 eq) and bis(4-nitrophenyl) carbonate (1.23 g, 4.04 mmol, 2.0 eq), and then stirred at 25° C. for 1 hr. The reaction mixture was quenched by addition H₂O 30 mL at 0° C., and then extracted with DCM (10 mL×3). The combined organic layers were washed with brine 20 mL, dried over Na₂SO₄, filtered, and concentrated under reduced pressure to give a residue. The residue was purified by flash silica gel chromatography (Biotage®; 20 g SepaFlash® Silica Flash Column, Eluent of 10-30% Ethyl acetate/petroleum ether gradient @ 40 mL/min). [4-[[(2S)-2-[bis(tert-butoxycarbonyl)amino]propanoyl]-tert-butoxycarbonyl-amino]phenyl]methyl (4-nitrophenyl) carbonate, Int-AF, (1.71 g, crude) was obtained as colorless oil: LC/MS [M+Na] 682.3 (calculated); LC/MS [M+Na] 682.2 (observed).

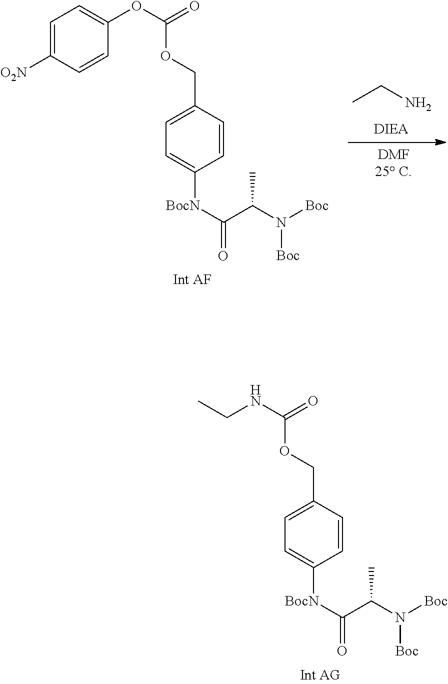

To a solution of ethanamine (315 mg, 3.87 mmol, 457 uL, 1.5 eq, HCl) in DMF (15 mL) was added DIEA (1.67 g, 12.9 mmol, 2.24 mL, 5.0 eq) and [4-[[(25)-2-[bis(tert-butoxycarbonyl)amino]propanoyl]-tert-butoxycarbonyl-amino]phenyl]methyl (4-nitrophenyl) carbonate, Int-AF, (1.70 g, 2.58 mmol, 1.0 eq) at 0° C. The mixture was stirred at 25° C. for 1 hr. The reaction mixture was quenched by addition H₂O 20 mL at 0° C., then extracted with EtOAc (20 mL×3). The combined organic layers were washed with brine 20 mL, dried over Na₂SO₄, filtered, and concentrated under reduced pressure to give a residue. The residue was purified by flash silica gel chromatography (Biotage®; 20 g SepaFlash® Silica Flash Column, Eluent of 30-50% Ethyl acetate/petroleum ether gradient @ 40 mL/min). tert-butyl N-[(2S)-2-[bis(tert-butoxycarbonyl)amino]propanoyl]-N-[4-(ethylcarbamoyloxymethyl)phenyl]carbamate, Int-AG, (1.14 g, 2.02 mmol, 78.21% yield) was obtained as a white solid: LC/MS [M+Na] 588.3 (calculated); LC/MS [M+Na] 588.3 (observed).

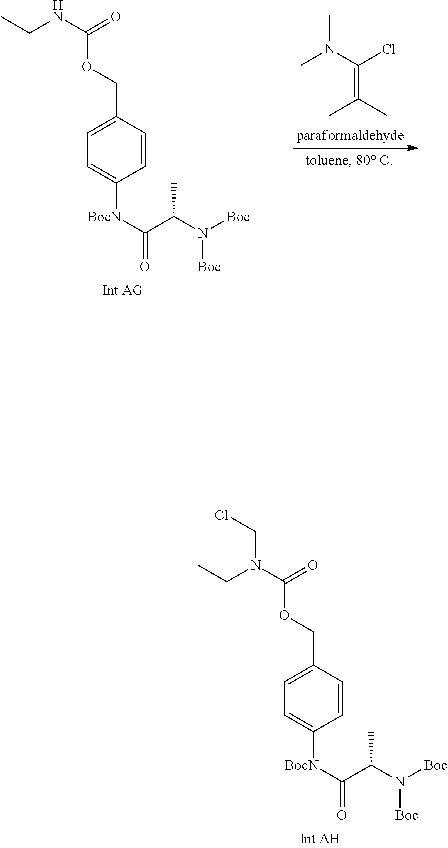

To a solution of tert-butyl N-[(2S)-2-[bis(tert-butoxycarbonyl)amino]propanoyl]-N-[4-(ethylcarbamoyloxymethyl)phenyl]carbamate, Int-AG, (1.00 g, 1.77 mmol, 1.0 eq) in toluene (10 mL) was added paraformaldehyde (3.15 g, 2.65 mmol, 1.5 eq) and 1-chloro-N,N,2-trimethyl-prop-1-en-1-amine (1.18 g, 8.84 mmol, 1.17 mL, 5.0 eq). The mixture was stirred at 80° C. for 1 hr. The reaction mixture was filtered and concentrated under reduced pressure to give a residue. tert-butyl N-[(2S)-2-[bis(tert-butoxycarbonyl)amino]propan oyl]-N-[4-[[chloromethyl(ethyl)carbamoyl]oxymethyl]phenyl]carbamate, Int-AH, (2 g, crude) was obtained as colorless oil.

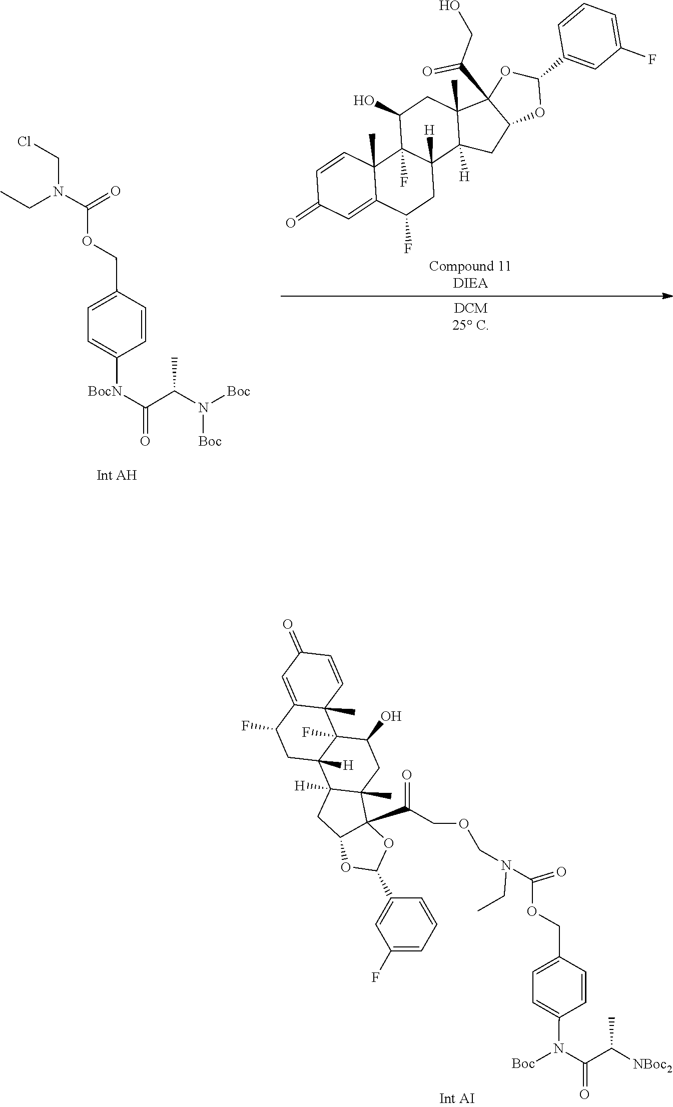

To a solution of tert-butyl N-[(2S)-2-[bis(tert-butoxycarbonyl)amino]propanoyl]-N-[4-[[chloromethyl(ethyl)carbamoyl]oxymethyl]phenyl]carbamate, Int-AH, (1.18 g, 1.93 mmol, 2.0 eq) in DCM (15 mL) was added DIEA (371 mg, 2.87 mmol, 0.5 mL, 2.98 eq) and (1S,2S,4R,6R,8S,9 S,11S,12R,13S,19S)-12,19-difluoro-6-(3-fluorophenyl)-11-hydroxy-8-(2-hydroxyacetyl)-9,13-dimethyl-5,7-dioxapentacyclo[10.8.0.02,9.04,8.013,18]icosa-14,17-dien-16-one (500 mg, 964.28 μmol, 1.0 eq), and then stirred at 25° C. for 12 hrs. The reaction mixture was quenched by addition H₂O 50 mL at 0° C., and then extracted with DCM (20 mL×3). The combined organic layers were washed with brine 20 mL, dried over Na₂SO₄, filtered, and concentrated under reduced pressure to give a residue. The residue was purified by flash silica gel chromatography (Biotage®; 20 g SepaFlash® Silica Flash Column, Eluent of 50˜70% Ethyl acetate/petroleum ether gradient @ 45 mL/min). tert-butyl N-[(2S)-2-[bis(tert-butoxycarbonyl)amino]propanoyl]-N-[4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04, 8.013,18]icosa-14,17-dien-8-yl]-2-oxo-ethoxy]methyl-ethyl-carbamoyl]oxymethyl]phenyl]carbamate, Int-AI, (658 mg, 600.26 μmol, 62.25% yield) was obtained as colorless oil: LC/MS [M+Na] 1118.5 (calculated); LC/MS [M+Na] 1118.3 (observed).

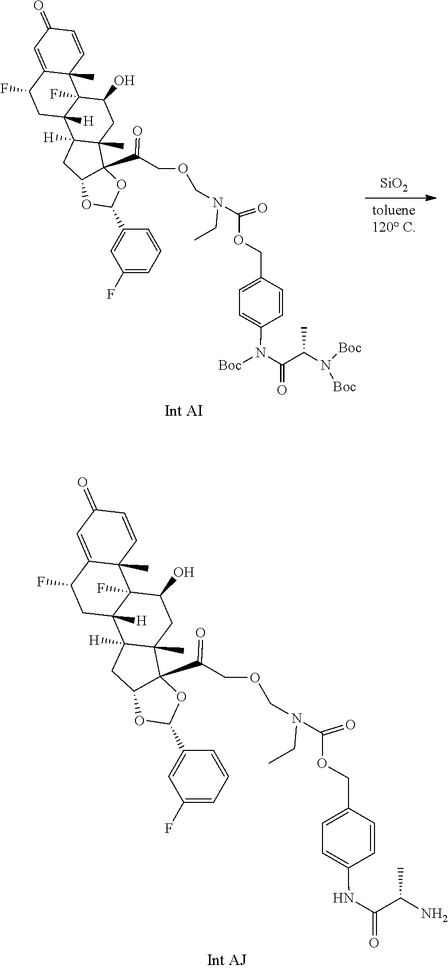

To a solution of tert-butyl N-[(2S)-2-[bis(tert-butoxycarbonyl)amino]propanoyl]-N-[4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]phenyl]carbamate, Int-AI, (0.96 g, 875 μmol, 1.0 eq) in toluene (15 mL) was added SiO₂ (10.0 g, 166 mmol, 190 eq). The mixture was stirred at 120° C. for 1 hr. The reaction mixture was filtered, SiO₂ was washed with MeOH (20 mL×2), and the filtrate was concentrated under reduced pressure to give a residue. [4-[[(2S)-2-aminopropanoyl]amino]phenyl]methylN-[[2-[(1S,2S,4R,6R,8S,9S,11S,12R,13S,19S)-12,19-difluoro-6-(3-fluorophenyl)-11-hydroxy-9,13-dimethyl-16-oxo-5,7dioxapentacyclo[10.8.0.02,9.04,8.013,18]icosa-14,17-dien-8-yl]-2-oxo-ethoxy]methyl]-N-ethyl-carbamate, Int-AJ, (500 mg, 628.27 μmol, 71.74% yield) was obtained as colorless oil: LC/MS [M+H] 796.3 (calculated); LC/MS [M+H] 796.4 (observed).

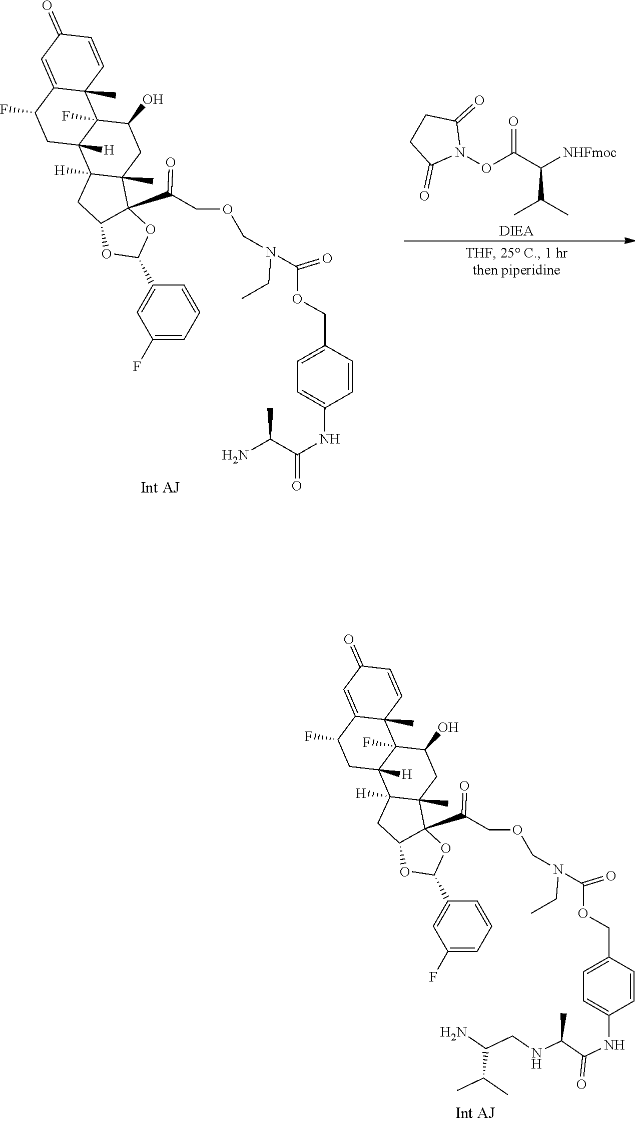

To a solution of [4-[[(2S)-2-aminopropanoyl]amino]phenyl]methyl N-[[2-[(1S,2S,4R,6R,8S,9S,11S,12R,13S,19S)-12,19-difluoro-6-(3-fluorophenyl)-11-hydroxy-9,13-dimeth yl-16-oxo-5,7-dioxapentacyclo[10.8.0.02, 9.04, 8.013,18]icosa-14,17-dien-8-yl]-2-oxo-ethoxy]methyl]-N-ethyl-carbamate, Int-AJ, (200 mg, 251 μmol, 1.0 eq) in THE (2 mL) was added DIEA (97.4 mg, 754 μmol, 131 uL, 3.0 eq) and (2,5-dioxopyrrolidin-1-yl) (2S)-2-(9H-fluoren-9-ylmethoxycarbonylamino)-3-methyl-butanoate (164 mg, 377 μmol, 1.5 eq), and then stirred at 25° C. for 1 hr. piperidine (86.2 mg, 1.01 mmol, 0.10 mL, 5.66 eq) was added. The mixture was stirred at 25° C. for another 1 hr. The mixture was purified by prep-HPLC (column: Phenomenex Luna 80×30 mm×3 um; mobile phase: [water(TFA)-ACN]; B %: 30%-65%, 8 min). [4-[[(2S)-2 [[(2S)-2-amino-3-methyl-butanoyl]amino]propanoyl]amino]phenyl]methyl N-[[2-[(1S,2S,4R,6R,8 S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl]-N-ethyl-carbamate, Int-AK, (100 mg, 44.69 μmol, 24.97% yield, 40% purity) was obtained as a light yellow solid: LC/MS [M+H] 895.4 (calculated); LC/MS [M+H] 895.5 (observed).

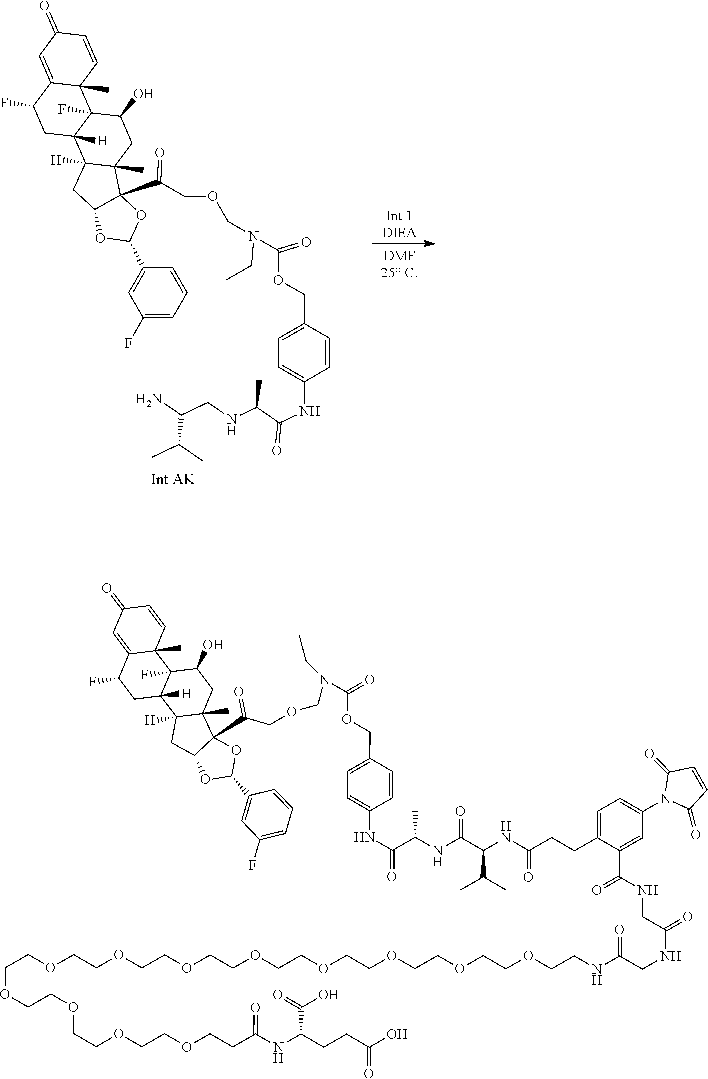

To a solution of [4-[[(2S)-2-[[(2S)-2-[[(2S)-2-amino-3-methyl-butanoyl]amino]propanoyl]amino]phenyl]methyl N-[[2-[(1S,2S,4R,6R,8S,9S,11 S,12R,13 S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl]-N-ethyl-carbamate, Int-AA, (94.3 mg, 51.4 μmol, 55% purity, 1.0 eq, TFA) in DMF (0.7 mL) was added DIEA (19.9 mg, 154 μmol, 26.8 uL, 3.0 eq) and (2S)-2-[3-[2-[2-[2-[2-[2-[2-[2-[2-[2-[2-[2-[2-[[2-[2-[[5-(2,5-dioxopyrrol- 1-yl)-2-[3-oxo-3-(2,3,5,6-tetrafluorophenoxy)propyl]benzoyl]amino]ethylamino]acetyl]amino]ethoxy]ethoxy]ethoxy]ethoxy]ethoxy]ethoxy]ethoxy]ethoxy]ethoxy]ethoxy]ethoxy]ethoxy]propanoylamino]pentanedioic acid, Int-1, (78.0 mg, 56.5 μmol, 1.1 eq, TFA) at 0° C. The mixture was stirred at 25° C. for 1 hr. The reaction mixture was quenched with HCOOH until pH=˜7. The reaction mixture was purified by prep-HPLC (column: Phenomenex Luna C₁₈ 200×40 mm×10 um; mobile phase: [water(FA)-ACN]; B %: 35%-65%, 8 min). (2S)-2-[3-[2-[2-[2-[2-[2-[2-[2-[2-[2-[2-[2-[2-[[2-[[2-[[2-[3-[[(1S)-1-[[(1S)-2-[4-[[[2-[(1S,2S,4R,6R,8S,9S,11S,12R,13S,19S)-12,19-Difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methyl-ethyl-carbamoyl]oxymethyl]anilino]-1-methyl-2-oxo-ethyl] carbamoyl]-2-methy 1-propyl] amino]-3-oxo-propyl]-5-(2,5-dioxopyrrol-1-yl)benzoyl]amino]acetyl] amino]-acetyl]amino]ethoxy]ethoxy]ethoxy]ethoxy]ethoxy]ethoxy]ethoxy]ethoxy]-ethoxy]ethoxy]ethoxy]ethoxy]propanoylamino]pentanedioic acid (21.9 mg, 10.85 μmol, 21.11% yield, 99.51% purity) was obtained as a white solid: ¹H NMR (400 MHz, DMSO-d₆) δ 9.97-9.80 (m, 1H), 8.70-8.62 (m, 1H), 8.27-8.16 (m, 2H), 7.92-7.85 (m, 2H), 7.64-7.16 (m, 12H), 7.14-7.08 (m, 2H), 6.29 (dd, J=1.6, 10.4 Hz, 1H), 6.12 (s, 1H), 5.72-5.47 (m, 2H), 5.05-4.89 (m, 3H), 4.86-4.57 (m, 3H), 4.38-4.24 (m, 2H), 4.19-4.17 (m, 2H), 4.12 (t, J=7.6 Hz, 1H), 3.92-3.86 (m, 2H), 3.74-3.69 (m, 2H), 3.60-3.54 (m, 3H), 3.49-3.44 (m, 44H), 3.38 (t, J=5.6 Hz, 3H), 3.31-3.27 (m, 1H), 3.22-3.19 (m, 2H), 3.01-2.91 (m, 2H), 2.38-2.26 (m, 4H), 2.20-2.09 (m, 1H), 1.99-1.87 (m, 3H), 1.78-1.64 (m, 4H), 1.47 (s, 3H), 1.27 (d, J=7.2 Hz, 3H), 1.06 (t, J=6.8 Hz, 3H), 0.87-0.74 (m, 9H); HPLC: 99.51% (220 nm), 98.86% (254 nm); LC/MS [M+H] 2008.9 (calculated); LC/MS [M+H] 2008.9 (observed).

### Example 75. Preparation of GR Agonist-Linker Compounds

The compounds in Table 11 below were prepared in a manner similar to that described Example 74 using the appropriate compounds as starting material.

TABLE 11
GR Agonist-Linker Compounds

| Structure | MS/¹H NMR |
|---|---|
| 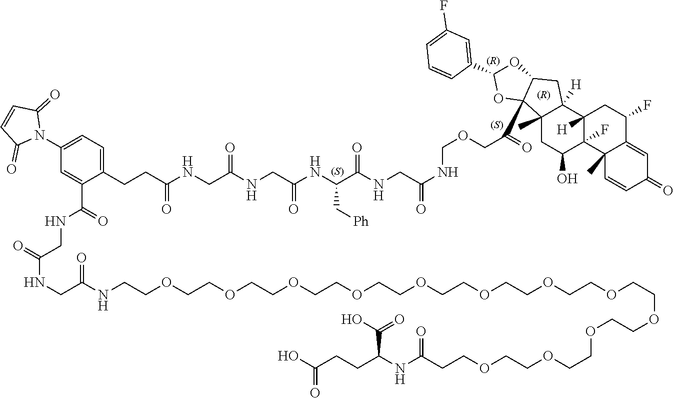 | MS m/z [M −H]⁻ (ESI): 1977.90; ¹H NMR (300 MHz, Methanol-d₄) δ 0.79 (d, J = 7.1 Hz, 3H), 0.89 (s, 3H), 1.08 (t, J = 7.1 Hz, 2H), 1.13-1.37 (m, 7H), 1.47 (s, 4H), 1.62-1.76 (m, 3H), 1.73- 1.89 (m, 1H), 2.11 (d, J = 12.7 Hz, 3H), 2.24 (t, J = 7.2 Hz, 3H), 2.31-2.45 (m, 2H), 2.49-2.62 (m, 2H), 2.78-3.16 (m, 4H), 3.28 (d, J = 5.4 Hz, 2H), 3.40-3.48 (m, 2H), 3.48- 3.58 (m, 36H), 3.56-3.74 (m, 6H), 3.79 (d, J = 11.7 Hz, 3H), 3.98 (s, 2H), 4.15-4.30 (m, 2H), 4.30-4.45 (m, 2H), 4.55- 4.77 (m, 4H), 4.95 (d, J = 4.3 Hz, 1H), 5.39 (s, 1H), 5.51 (s, 1H), 6.13- 6.24 (m, 2H), 6.88 (s, |
| | 1H), 7.01 (t, J = 8.9 Hz, |
| | 1H), 7.07-7.25 (m, 7H), |
| | 7.23-7.32 (m, 1H), 7.32- |
| | 7.49 (m, 2H) |
| 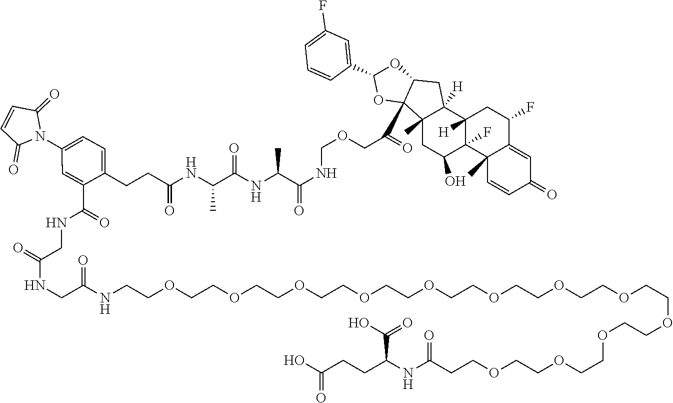 | MS m/z [M − H]⁻ (ESI): 1801.60; ¹H NMR (300 MHz, Methanol-d₄) δ: 0.98 (s, 3H), 1.27-1.34 (m, 6H), 1.57 (s, 4H), 1.80-1.91 (m, 4H), 2.10- 2.20 (m, 2H), 2.37-2.39 (m, 4H), 2.63-2.70 (m, 5H), 3.13-3.20 (m, 2H), 3.37-3.40 (m, 3H), 3.52- 3.59 (m, 6H), 3.61-3.62 (m, 37H), 3.72-3.74 (m, 2H), 3.92 (s, 3H), 4.09 (s, 2H), 4.17-4.23 (m, 3H), 4.26-4.43 (m, 2H), 4.66-4.75 (m, 4H), 5.03- 5.04 (m, 1H), 5.50-5.58 (m, 2H), 6.31-6.34 (m, 2H), 6.97 (s, 2H), 7.10- 7.20 (m, 2H), 7.32-7.47 (m, 6H). |
| 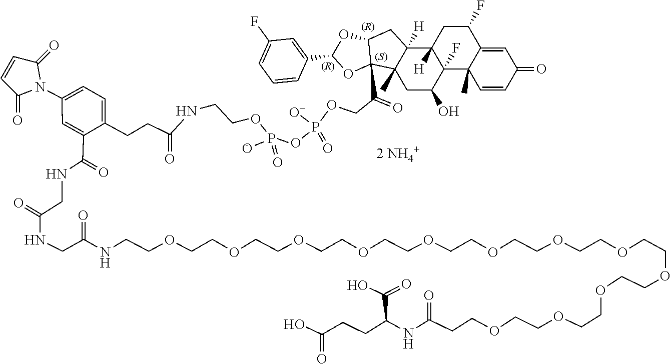 | MS m/z [M − H]⁻ (ESI): 1834.50; ¹H NMR (300 MHz, Methanol-d₄) δ: 0.75-1.12 (m, 3H), 1.20- 1.43 (m, 1H), 1.51-1.68 (m, 4H), 1.70-1.84 (m, 2H), 1.85-2.06 (m, 2H), 2.05-2.42 (m, 6H), 2.45- 2.61 (m, 4H), 2.69 (d, J = 31.9 Hz, 1H), 3.08 (s, 2H), 3.41 (t, J = 5.7 Hz, 2H), 3.53-3.59 (m, 2H), 3.59-3.66 (m, 43H), 3.74 (t, J = 6.2 Hz, 2H), 3.90 (d, J = 21.1 Hz, 4H), 4.11 (d, J = 4.1 Hz, 2H), 4.23 (dd, J = 7.9 Hz, 4.2 Hz, 1H), 4.32 (d, J = 9.7 Hz, 1H), 4.92-5.16 (m, 4H), 5.34-5.54 (m, 1H), 5.60 (s, 1H), 5.90-6.03 (m, 1H), 6.26-6.63 (m, |
| | 3H), 6.82-7.46 (m, 6H), |
| | 7.56-7.66 (m, 1H), 7.73- |
| | 7.88 (m, 1H) |

### Example 76. Preparation of 4-(2-((S)-10-benzyl-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-1,6,9,12,15,18-hexaoxo-3-oxa-5,8,11,14,17-pentaazaicosan-20-yl)-5-(2,5-dioxo-2,5-dihydro-1H-pyrrol-1-yl)phenoxy)butanoic acid

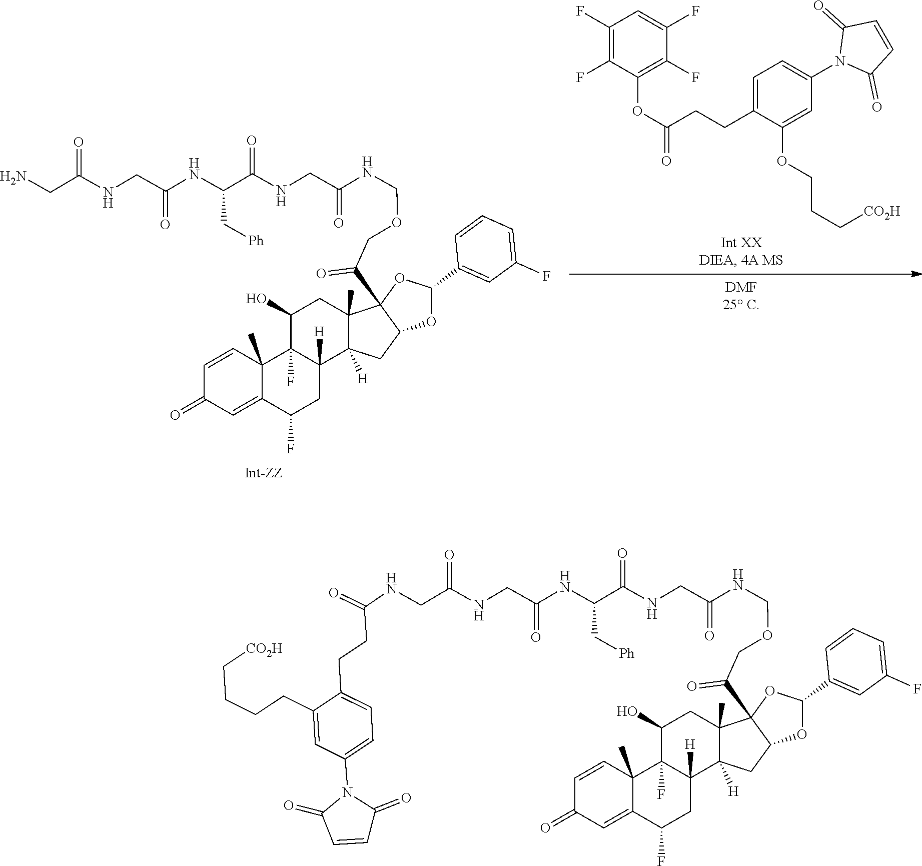

A solution of Int XX (120.0 mg, 0.20 mmol, 1.0 equiv.) in N,N-dimethylformamide (2.0 mL) was treated with N N-diisopropylethylamine (52.3 m 0.40 mmol, 2.0 equiv. at room temperature. To the above mixture was added Int ZZ (192.6 mg, 0.22 mmol, 1.1 equiv.) at 25° C. The resulting mixture was stirred for additional 5 h at 25° C. The crude product was purified by Prep-HPLC with the following conditions (acetonitrile/water with 0.05% formic acid). 64 mg (26%) of desired product was obtained as a white solid. [M−H]⁻ (ESI): 1193.40. ¹H NMR (300 MHz, Methanol-d₄) δ 0.98 (s, 3H), 1.57 (s, 4H), 1.64-1.90 (m, 3H), 1.99-2.23 (m, 3H), 2.24-2.4 (m, 2H), 2.42-2.82 (m, 5H), 2.86-3.08 (m, 3H), 3.20 (dd, J=13.9 Hz, 5.8 Hz, 1H), 3.68-3.95 (m, 6H), 4.02 (t, J=6.1 Hz, 2H), 4.24-4.38 (m, 1H), 4.39-4.52 (m, 2H), 4.62 (d, J=28.1 Hz, 1H), 4.70-4.81 (m, 2H), 5.04 (d, J=4.4 Hz, 1H), 5.37-5.71 (m, 2H), 6.16-6.35 (m, 2H), 6.81 (dd, J=8.0 Hz, 1.9 Hz, 1H), 6.88-6.98 (m, 3H), 7.03-7.17 (m, 1H), 7.14-7.44 (m, 10H).

### Example 77. Preparation of GR Agonist-Linker Compounds

The compounds in Table 12 below were prepared in a manner similar to that described Example 76 using the appropriate compounds as starting material.

TABLE 12
GR Agonist-Linker Compounds

| Structure | MS/¹H NMR |
|---|---|
| 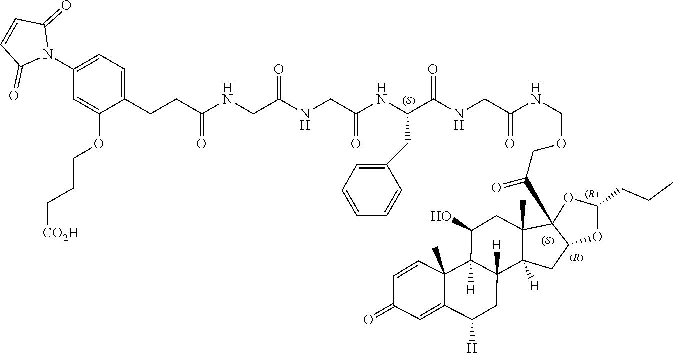 | MS m/z [M − H]⁻ (ESI): 1105.40; ¹H NMR (300 MHz, Methanol- d₄) δ: 0.88-0.98 (m, 6H), 0.99- 1.02 (m, 1H), 1.03-1.19 (m, 1H), 1.27 (s, 1H), 1.39-1.44 (m, 2H), 1.47 (s, 3H), 1.51-1.64 (m, 4H), 1.66-1.73 (m, 1H), 1.81-1.89 (m, 3H), 2.02-2.33 (m, 4H), 2.31- 2.39 (m, 1H), 2.46-2.53 (m, 2H), 2.58-2.69 (m, 3H), 2.91-3.09 (m, 3H), 3.11-3.19 (m, 1H), 3.21- 3.29 (m, 1H), 3.71-2.91 (m, 6H), 3.92-4.11 (m, 2H), 4.39-4.41 (m, 1H), 4.48-4.58 (m, 2H), 4.61- 4.66 (m, 1H), 4.69-4.79 (m, 2H), 5.99 (s, 1H), 6.12-6.28 (m, 1H), 6.76-6.88 (m, 1H), 6.89-6.99 (m, 3H), 7.11-7.31 (m, 6H), 7.32- 7.44 (m, 1H) |
| 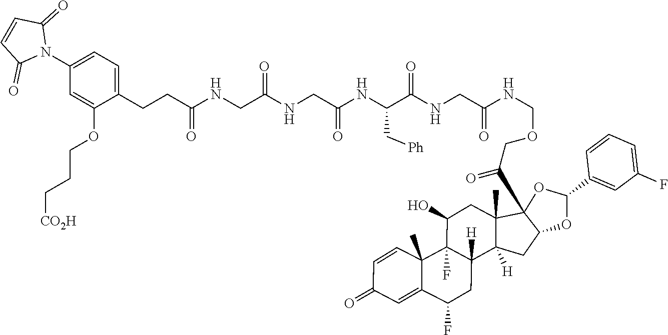 | [M − H]⁻ (ESI): 1193.40. ¹H NMR (300 MHz, Methanol-d₄) δ 0.98 (s, 3H), 1.57 (s, 4H), 1.64-1.90 (m, 3H), 1.99-2.23 (m, 3H), 2.24- 2.4 (m, 2H), 2.42-2.82 (m, 5H), 2.86-3.08 (m, 3H), 3.20 (dd, J = 13.9 Hz, 5.8 Hz, 1H), 3.68-3.95 (m, 6H), 4.02 (t, J = 6.1 Hz, 2H), 4.24-4.38 (m, 1H), 4.39-4.52 (m, 2H), 4.62 (d, J = 28.1 Hz, 1H), 4.70-4.81 (m, 2H), 5.04 (d, J = 4.4 Hz, 1H), 5.37-5.71 (m, 2H), 6.16-6.35 (m, 2H), 6.81 (dd, J = 8.0 Hz, 1.9 Hz, 1H), 6.88-6.98 (m, 3H), 7.03-7.17 (m, 1H), 7.14- 7.44 (m, 10H). |

### Example 78. Preparation of 34-(2-((10S)-10-benzyl-1-((2S,6aS,6bR,7S,8aS,8bS,10R,11aR,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-1,6,9,12,15,18-hexaoxo-3-oxa-5,8,11,14,17-pentaazaicosan-20-yl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid

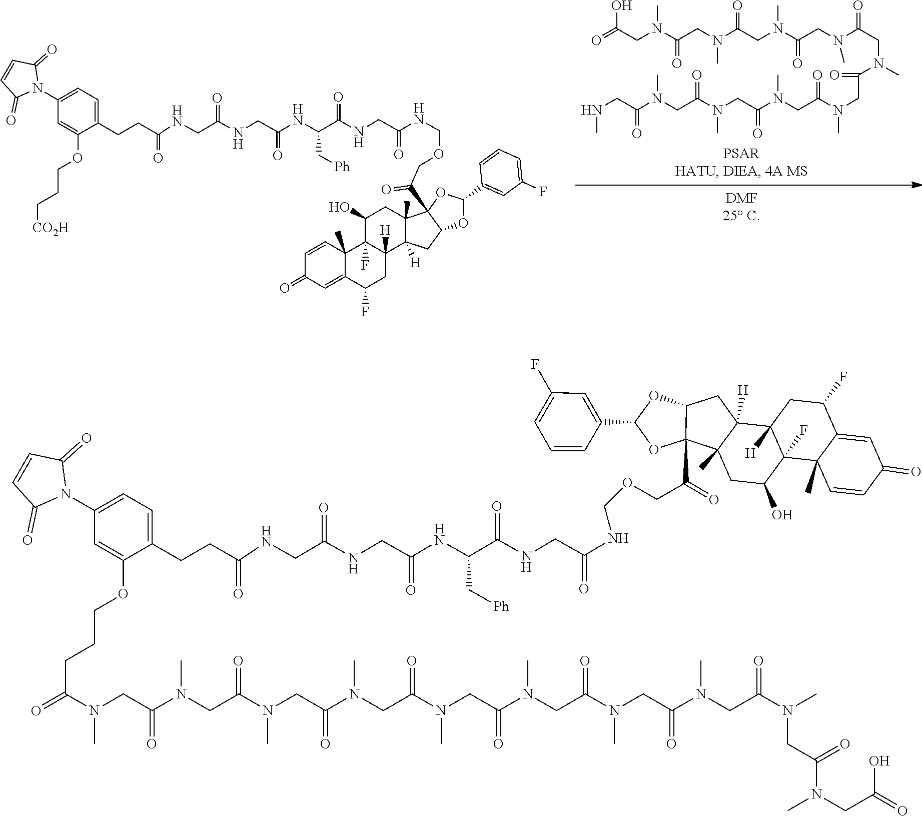

To a stirred solution of starting material (64.0 mg, 0.05 mmol, 1 equiv.) and PSAR (46.8 mg, 0.06 mmol, 1.2 equiv.) in N,N-dimethylformamide(1.0 mL) were added 2-(7-Azabenzotriazol-1-yl)-N,N,N′,N′-tetramethyluronium hexafluorophosphate (30.5 mg, 0.08 mmol, 1.5 equiv.) and N,N-diisopropylethylamine (17.3 mg, 0.13 mmol, 2.5 equiv.) at room temperature under nitrogen atmosphere. The resulting solution was stirred for 3 h at 25° C. The crude product was purified by Prep-HPLC with the following conditions (IntelFlash-1): Column: Xselect CSH C₁₈ OBD, 30×150 mm, 5 μm; Mobile Phase A: water (0.05% formic acid), Mobile Phase B: acetonitrile; Flow rate: 60 mL/min; Gradient: 32% B to 62% B in 9 min, 62% B; Wave Length: 254 nm. The resulting mixture was lyophilized in a cool and dry place. 24.3 mg (25% yield) of desired product was obtained as a white solid. [M−H]⁻ (ESI): 1904.75; ¹H NMR (300 MHz, Methanol-d₄) δ 0.98 (s, 4H), 1.29 (s, 1H), 1.56 (s, 4H), 1.79 (d, J=13.8 Hz, 3H), 2.01-2.24 (m, 4H), 2.33 (s, 2H), 2.52-2.78 (m, 5H), 2.81-3.24 (m, 33H), 3.61-3.90 (m, 6H), 3.90-4.23 (m, 12H), 4.24-4.53 (m, 11H), 4.66-4.81 (m, 3H), 5.03 (d, J=4.4 Hz, 1H), 5.60 (s, 2H), 6.28 (d, J=11.7 Hz, 2H), 6.74-7.16 (m, 5H), 7.16-7.42 (m, 10H).

### Example 79. Preparation of GR Agonist-Linker Compounds

The compounds in Table 13 below were prepared in a manner similar to that described Example 78 using the appropriate compounds as starting material.

TABLE 13
GR Agonist-Linker Compounds

| Structure | MS/¹H NMR |
|---|---|
| 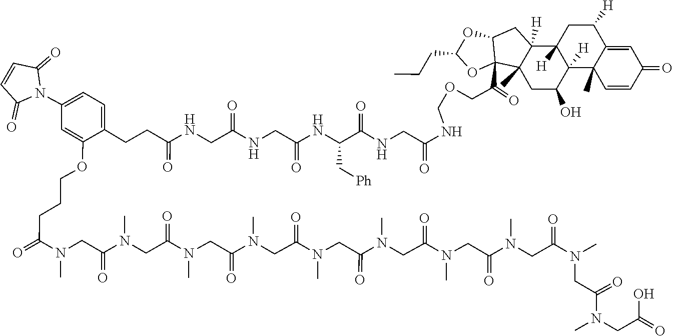 | MS m/z [M − H]⁻ (ESI): 1816.80; ¹H NMR (400 MHZ, Methanol- d₄) δ: 0.79-0.88 (m, 6H), 0.89- 0.92 (m, 1H), 0.94-1.04 (m, 1H), 1.13-1.24 (m, 5H), 1.26-1.34 (m, 3H), 1.37 (s, 3H), 1.42-1.51 (m, 4H), 1.58-1.62 (m, 1H), 1.69- 1.71 (m, 2H), 1.91-2.19 (m, 5H), 2.21-2.29 (m, 1H), 2.46-2.61 (m, 4H), 2.79-2.88 (m, 15H), 2.89- 2.99 (m, 13H), 3.11-3.18 (m, 1H), 3.66-3.81 (m, 6H), 3.91- 4.01 (m, 4H), 4.02-4.18 (m, 7H), 4.21-4.24 (m, 3H), 4.26-4.32 (m, 5H), 4.39-4.48 (m, 2H), 4.49- 4.58 (m, 3H), 4.61-4.68 (m, 3H), 4.71-4.76 (m, 2H), 5.89 (s, 1H), 5.99-6.22 (m, 1H), 6.61-6.78 (m, 1H), 6.79-6.98 (m, 3H), 7.01- 7.18 (m, 2H), 7.19-7.24 (m, 4H), 7.26-7.38 (m, 1H). |

### Example 80. Preparation of 4-(2-((7S,10S)-1-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-7,10-dimethyl-1,6,9,12-tetraoxo-3-oxa-5,8,11-triazatetradecan-14-yl)-5-(2,5-dioxo-2,5-dihydro-1H-pyrrol-1-yl)phenoxy)butanoic acid

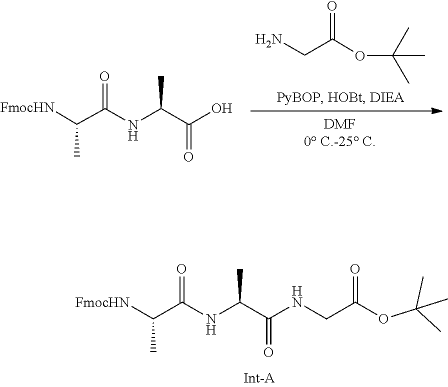

To a stirred solution of (2S)-2-[(2S)-2-{[(9H-fluoren-9-ylmethoxy) carbonyl]amino}propanamido] propanoic acid (1.0 g, 2.6 mmol, 1.0 equiv.) in N,N-dimethylformamide (5.0 mL) with an inert atmosphere of nitrogen, was added tert-butyl 2-aminoacetate (343.0 mg, 2.6 mmol, 1.0 equiv.), N,N-diisopropylethylamine (675.9 mg, 5.2 mmol, 2.0 equiv.), benzotriazol-1-yl-oxytripyrrolidinophosphonium hexafluorophosphate (530.0 mg, 3.9 mmol, 1.5 equiv.) at 0° C. The resulting solution was stirred for 4 h at 25° C., diluted with water (100 mL). The resulting mixture was extracted with dichloromethane (3×100 mL) and organic layers were washed with saturated sodium chloride solution (3×100 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 1.0 g (77%) of Int-A was obtained as a white solid. MS m/z [M+H]⁺ (ESI): 496.23.

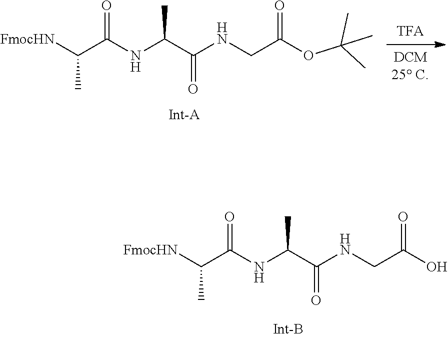

To a solution of Int-A (1.0 g, 2.0 mmol, 1.0 equiv.) in dichloromethane (20.0 mL) with an inert atmosphere of nitrogen, was added trifluoroacetic acid (10.0 mL) at 25° C. The resulting solution was stirred for 5 h at 25° C. and concentrated under vacuum. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 800 mg (90%) of Int-B was obtained as a yellow oil. MS m/z [M−H]⁻ (ESI): 438.17.

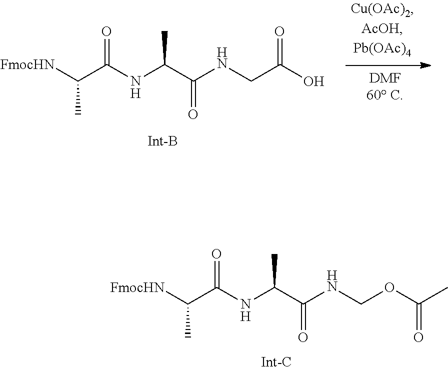

To a stirred solution of Int-B (800.0 mg, 1.8 mmol, 1.0 equiv.) and cupric acetate monohydrate (33.0 mg, 0.2 mmol, 0.1 equiv.) in N,N-Dimethylformamide (10.0 mL) were added acetic acid (164.0 mg, 2.7 mmol, 1.5 equiv.) and Lead tetraacetate (4.0 g, 9.0 mmol, 5.0 equiv.) at room temperature under air atmosphere. The resulting mixture was stirred for additional 5 h at 60° C. The resulting solution was cooled to 25° C., diluted with water (100 mL). The resulting mixture was extracted with dichloromethane (3×100 mL) and organic layers were washed with saturated sodium chloride solution (3×100 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 425 mg (51%) of Int-D was obtained as a white solid. MS m/z [M+Na]⁺ (ESI): 476.17.

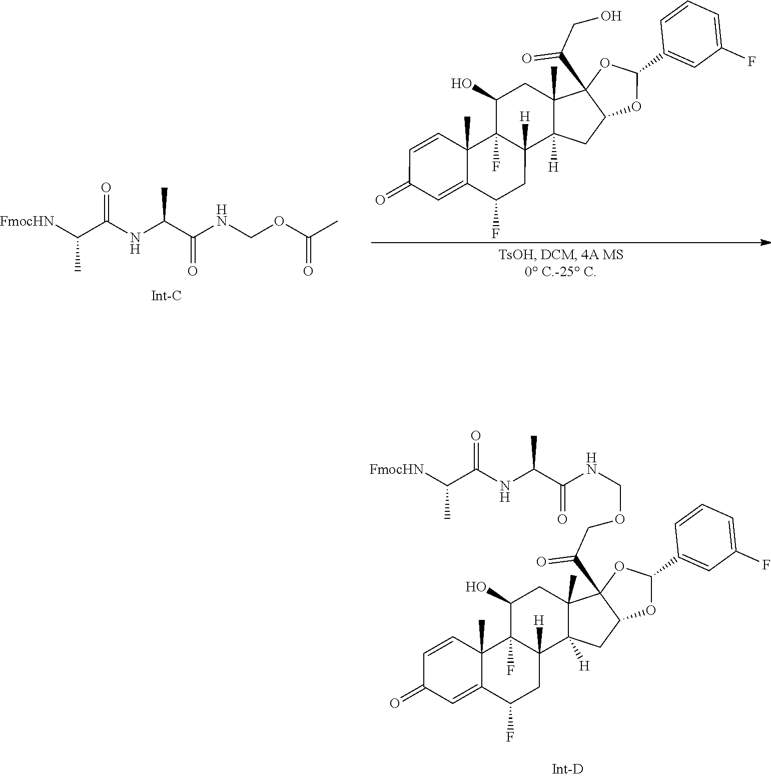

To a stirred solution of Int-C(425.0 mg, 0.94 mmol, 1.0 equiv.) and starting material (243.0 mg, 0.5 mmol, 0.5 equiv.) in dichloromethane (5.0 mL) were added p-toluenesulfonic acid (32.2 mg, 0.2 mmol, 0.20 equiv.) in portions at 0° C. under air atmosphere. The resulting mixture was stirred for additional 12 h at 25° C. The crude product was purified by reverse phase flash with the following conditions (column, C₁₈ silica gel; mobile phase, acetonitrile in water (10 mmol/L ammonium bicarbonate), 10% to 100% gradient in 20 min; detector, UV 254 nm.). 200 mg (47%) of Int-D was obtained as a white solid. MS m/z [M+H]⁺ (ESI): 912.36.

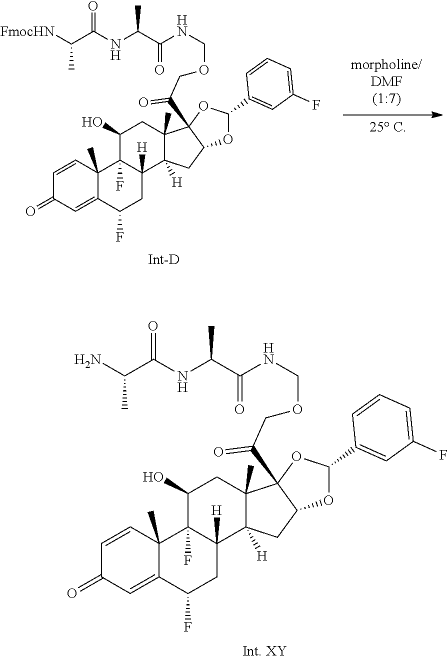

To a stirred solution of Int-D (200 mg, 0.2 mmol, 1.0 equiv.) in N, N-dimethylformamide (2.0 mL) were added morpholine (0.3 mL) at 25° C. under air atmosphere. The resulting mixture was stirred for additional 1 h at 25° C. The crude product was purified by reverse phase flash with the following conditions (column, C₁₈ silica gel; mobile phase, acetonitrile in water (0.1% trifluoroacetic acid), 10% to 100% gradient in 20 min; detector, UV 254 nm). 100 mg (66%) of Int XY was obtained as a white solid. MS m/z [M+H]⁺ (ESI): 690.29.

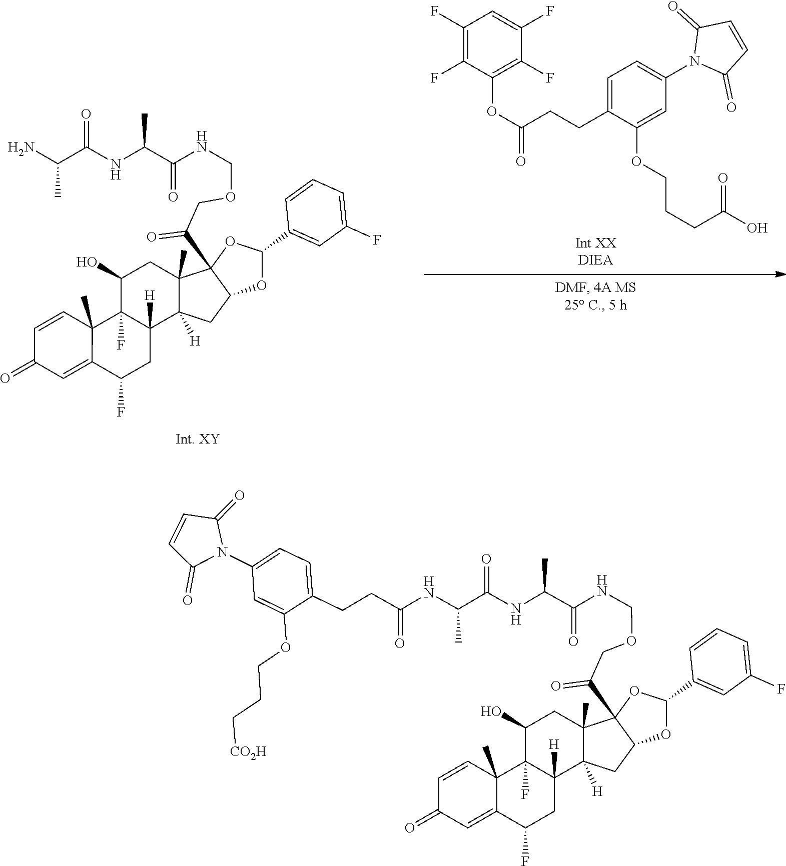

To a stirred solution of Int XX (200.0 mg, 0.40 mmol, 1.0 equiv.) and Int YY (417.7 mg, 0.60 mmol, 1.5 equiv.) in N,N-dimethylformamide (5.0 mL), was added N,N-diisopropylethylamine (156.5 mg, 1.20 mmol, 3.0 equiv.) dropwise in at 25° C. The resulting mixture was stirred for additional 5 h at 25° C. The crude product was purified by Flash with the following conditions (IntelFlash-1): Column, C₁₈ silica gel; mobile phase, water (with 5 mmol/L formic acid) and acetonitrile (10.0% acetonitrile up to 100.0% in 20 min); Detector, UV 254 nm. 150 mg (36%) of desired product was obtained as white solid: MS m/z [M+H]⁺ (ESI): 1019.38.

### Example 81. Preparation of GR Agonist-Linker Compounds

The compounds below were prepared in a manner similar to that described Example 80 using the appropriate compounds as starting material (Table 14).

TABLE 14
GR Agonist-Linker Compounds

| Structure | MS/¹H NMR |
|---|---|
| 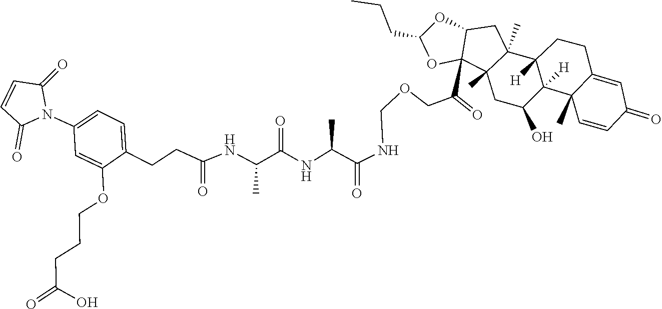 | |
| 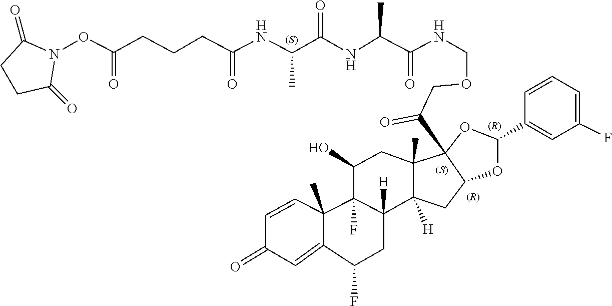 | MS m/z [M + H]⁺ (ESI): 901.30; ¹H NMR (300 MHz, Methanol- d₄) δ: 0.99 (s, 3H), 1.31-1.38 (m, 6H), 1.59 (s, 4H), 1.79-1.85 (m, 3H), 1.97-1.99 (m, 2H), 2.32- 2.46 (m, 6H), 2.48-2.50 (m, 2H), 2.61-2.68 (m, 3H), 2.82 (s, 2H), 4.20-4.44 (m, 4H), 4.66-4.77 (m, 2H), 5.04-5.06 (m, 1H), 5.59- 5.65 (m, 2H), 6.32-6.35 (m, 2H), 7.12-7.22 (m, 2H), 7.32-7.41 (m, 3H) |
| 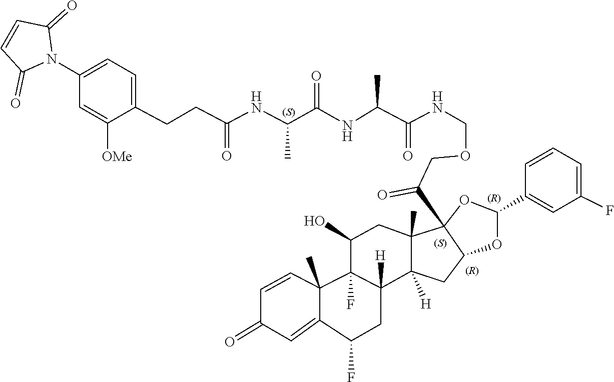 | MS m/z [M − H]⁻ (ESI): 945.30; ¹H NMR (400 MHZ, Methanol- d₄) δ: 0.99 (s, 3H), 1.27-1.29 (m, 3H), 1.35-1.36 (m, 3H), 1.56 (s, 4H), 1.78-1.81 (m, 3H), 2.15- 2.25 (m, 1H), 2.30-2.34 (m, 2H), 2.50-2.54 (m, 2H), 2.60-2.70 (m, 1H), 2.91-2.99 (m, 2H), 3.81 (s, 3H), 4.21-4.28 (m, 3H), 4.39- 4.44 (m, 1H), 4.68-4.77 (m, 3H), 5.04-5.05 (m, 1H), 5.50-5.58 (m, 2H), 6.30-6.33 (m, 2H), 6.79- 6.81 (m, 1H), 6.89-6.94 (m, 3H), 7.11-7.18 (m, 1H), 7.20-7.29 (m, 2H), 7.31-7.39 (m, 3H). |

### Example 82. Preparation of 34-(2-((7S,10S)-1-((2S,6aS,6bR,7S,8aS,8bS,10R,11aR,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-7,10-dimethyl-1,6,9,12-tetraoxo-3-oxa-5,8,11-triazatetradecan-14-yl)-5-(2,5-dioxo-2,5-dihydro-1H-pyrrol-1-yl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid

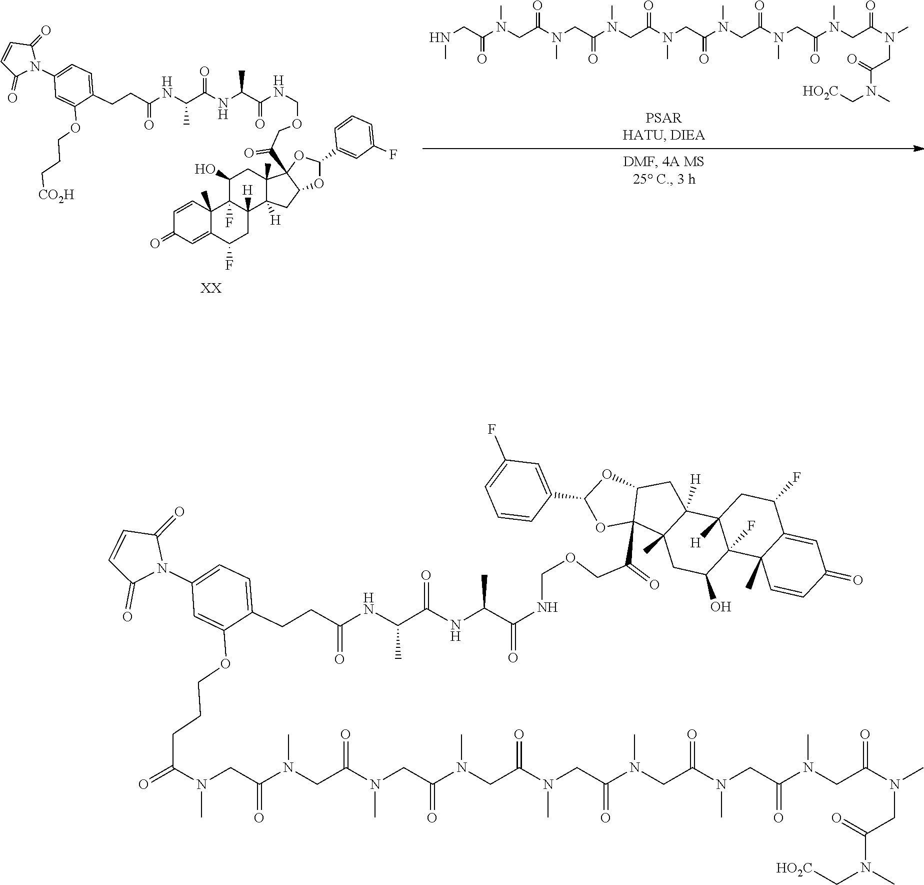

To a stirred solution of Compound XX (N-191096) (100.0 mg, 0.098 mmol, 1.0 equiv.) and HATU (2-(3H-[1,2,3]triazolo[4,5-b]pyridin-3-yl)-1,1,3,3-tetramethylisouronium) (55.97 mg, 0.147 mmol, 1.5 equiv.) in N,N-dimethylformamide (2.0 mL) were added N,N-diisopropylethylamine (38.05 mg, 0.294 mmol, 3.0 equiv.) and PSAR (5,8,11,14,17,20,23,26,29-nonamethyl-4,7,10,13,16,19,22,25,28-nonaoxo-2,5,8,11,14,17,20,23,26,29-decaazahentriacontan-31-oic acid) (143.04 mg, 0.196 mmol, 2.0 equiv.) dropwise at 25° C. under air atmosphere. The resulting mixture was stirred for additional 3 h at 25° C. The crude product was purified by Flash with the following conditions (IntelFlash-1): Column, C₁₈ silica gel; mobile phase, water and acetonitrile (10.0% acetonitrile up to 100.0% in 20 min); Detector, UV 254 nm. The resulting mixture was lyophilized in a cool and dry place. 20 mg (10%) of desired product was obtained as white solid: MS m/z [M+H]⁺ (ESI): 1728.05; ¹H NMR (400 MHz, Methanol-d₄) δ: 0.86-0.90 (m, 1H), 1.02 (s, 3H), 1.22-1.40 (m, 6H), 1.56 (s, 3H), 1.56-1.70 (m, 1H), 1.75-1.9 (m, 3H), 2.05-2.40 (m, 3H), 2.25-2.45 (m, 2H), 2.50-2.61 (m, 2H), 2.62-2.75 (m, 2H), 2.90-3.15 (m, 33H), 3.95-4.55 (m, 24H), 4.61-4.82 (m, 3H), 5.05-5.11 (m, 1H), 5.40-5.70 (m, 2H), 6.25-6.40 (m, 2H), 6.70-6.82 (m, 1H), 6.85-7.00 (m, 3H), 7.05-7.28 (m, 3H), 7.31-7.5 (m, 3H).

### Example 83. Preparation of GR Agonist-Linker Compounds

The compounds in Table 15 below were prepared in a manner similar to that described Example 82 using the appropriate compounds as starting material.

TABLE 15
GR Agonist-Linker Compounds

| Structure | MS/¹H NMR |
|---|---|
| 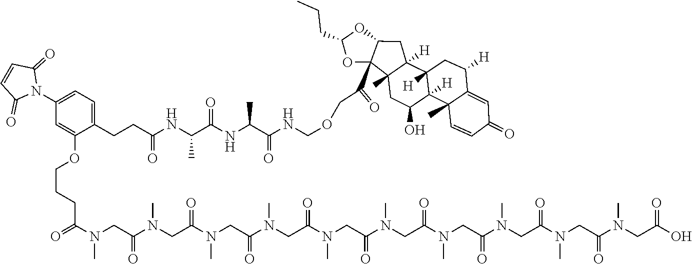 | |
| 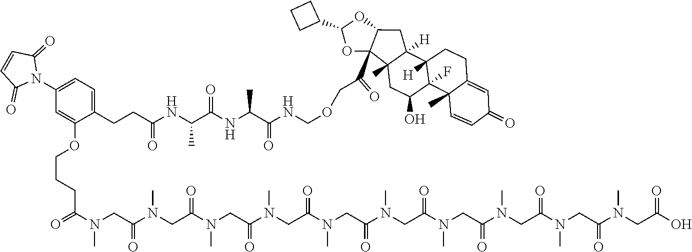 | MS m/z [M + H]⁺ (ESI): 1672.0; ¹H NMR (400 MHz, DMSO-d₆) δ 8.76-8.59 (m, 1H), 7.33-7.24 (m, 1H), 7.22-7.15 (m, 1H), 7.11 (s, 2H), 6.88 (br d, J = 8.8 Hz, 1H), 6.76 (br dd, J = 1.6, 7.6 Hz, 1H), 6.22 (dd, J = 1.6, 10.0 Hz, 1H), 6.01 (s, 1H), 4.74 (br s, 1H), 4.69- 4.42 (m, 4H), 4.38-4.14 (m, 13H), 4.13-3.86 (m, 12H), 3.06-2.67 (m, 32H), 2.61 (dt, J = 5.2, |
| | 13.2 Hz, 1H), 2.47-2.37 |
| | (m, 3H), 2.36-2.21 (m, |
| | 2H), 2.07-1.64 (m, |
| | 13H), 1.55 (br d, J = 8.4 |
| | Hz, 2H), 1.46 (s, 3H), |
| | 1.36-1.25 (m, 1H), |
| | 1.25-1.19 (m, 3H), |
| | 1.19-1.12 (m, 3H), 0.81 |
| | (s, 3H). |
| 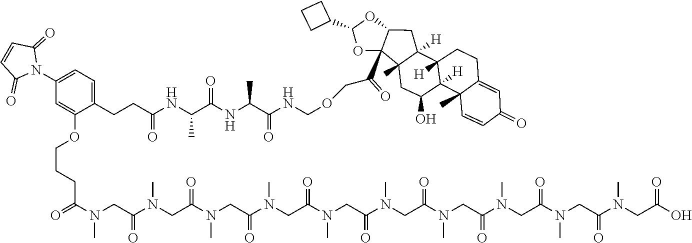 | MS m/z [M + Na]⁺ (ESI): 1675.9; ¹H NMR (400 MHZ, DMSO-d₆) δ 8.75-8.57 (m, 1H), 7.30 (br d, J = 10.0 Hz, 1H), 7.24-7.14 (m, 1H), 7.08 (s, 2H), 6.87 (br d, J = 9.2 Hz, 1H), 6.75 (br dd, J = 1.6, 7.6 Hz, 1H), 6.20- 6.10 (m, 1H), 5.91 (s, 1H), 4.72 (br d, J = 3.6 Hz, 1H), 4.67-4.39 (m, 4H), 4.37-4.14 (m, 13H), 4.12-3.82 (m, |
| | 12H), 3.06-2.62 (m, |
| | 33H), 2.47-2.37 (m, |
| | 3H), 2.34-2.19 (m, 2H), |
| | 2.13-1.66 (m, 13H), |
| | 1.65-1.43 (m, 3H), 1.35 |
| | (s, 3H), 1.25-1.18 (m, |
| | 3H), 1.18-1.10 (m, 3H), |
| | 1.01-0.82 (m, 2H), 0.79 |
| | (s, 3H). |
| 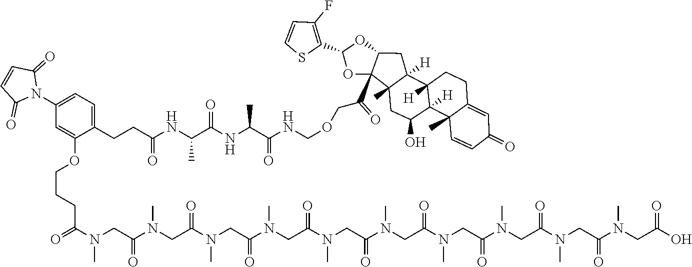 | MS m/z [M + Na]+ (ESI): 1722.0; ¹H NMR (400 MHZ, DMSO-d₆) δ8.74-8.60 (m, 1H), 7.57 (s, 1H), 7.32 (d, J = 9.6 Hz, 1H), 7.18 (dd, J = 3.2, 10.4 Hz, 1H), 7.12 (s, 2H), 6.97-6.86 (m, 2H), 6.80-6.71 (m, 1H), 6.21-6.14 (m, 1H), 5.96 (d, J = 18.8 Hz, 2H), 5.73 (s, 1H), 4.87 (d, J = 1.6 Hz, 1H), 4.71-4.50 (m, 4H), 4.31 (s, 6H), 4.22 (d, J = 14.4 Hz, 9H), 2.94-2.75 |
| | (m, 35H), 2.43-2.38 (m, |
| | 3H), 2.34-2.29 (m, 2H), |
| | 2.06 (s, 5H), 1.98-1.88 |
| | (m, 3H), 1.82-1.68 (m, |
| | 4H), 1.65 (s, 3H), 1.38 |
| | (s, 4H), 1.24-1.11 (m, |
| | 9H), 0.85 (s, 4H). |
| 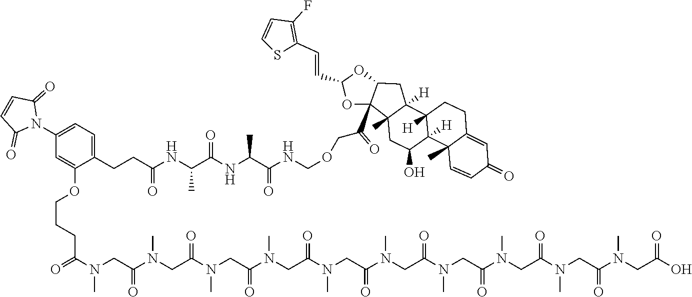 | MS m/z [M − H]⁻ (ESI): 1724.1; ¹H NMR (400 MHz, DMSO-d₆) δ7.51- 7.43 (m, 1H), 7.30 (d, J = 10.0 Hz, 1H), 7.21- 7.12 (m, 1H), 7.06 (s, 2H), 6.95 (d, J = 6.0 Hz, 1H), 6.90 (d, J = 16.0 Hz, 1H), 6.84 (d, J = 10.0 Hz, 1H), 6.76- 6.65 (m, 1H), 6.14 (d, J = 9.6 Hz, 1H), 5.91 (s, 1H), 5.87-5.76 (m, 1H), 5.12 (d, J = 6.4 Hz, 1H), 4.84-4.72 (m, 1H), 4.64-4.48 (m, 3H), 4.31-4.24(m, 5H), 4.24- 4.13 (m, 9H), 4.11-3.92 |
| | (m, 11H), 2.97-2.71 (m, |
| | 34H), 2.44-2.36 (m, |
| | 2H), 2.34-2.23 (m, 2H), |
| | 2.11-1.86 (m, 5H), 1.72 |
| | (s, 2H), 1.64-1.52 (m, |
| | 3H), 1.35 (s, 3H), 1.22- |
| | 1.13 (m, 6H), 1.04-0.91 |
| | (m, 2H), 0.80 (s, 3H). |
| 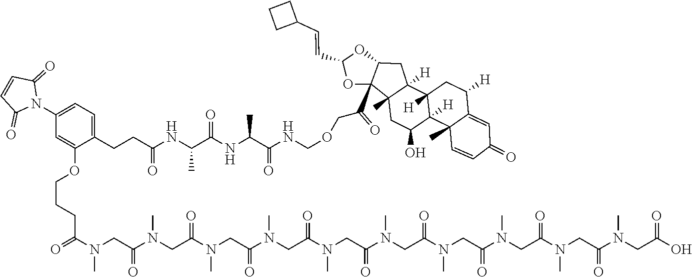 | MS m/z [M − H]⁻ (ESI): 1678.1; ¹H NMR (400 MHz, DMSO-d₆) δ7.31 (d, J = 10.0 Hz, 1H), 7.22-7.13 (m, 1H), 7.05 (s, 2H), 6.85 (d, J = 10.4 Hz, 1H), 6.78-6.70 (m, 1H), 6.21-6.11 (m, 1H), 6.05-5.96 (m, 1H), 5.92 (s, 1H), 5.32-5.22 (m, 1H), 4.91 (d, J = 6.8 Hz, 1H), 4.71 (d, J = 3.6 Hz, 1H), 4.65-4.40 (m, 3H), 4.38-3.96 (m, 24H), 3.01-2.66 (m, 36H), 2.45-2.26 (m, 4H), 2.09-1.90 (m, 6H), |
| | 1.82-1.64 (m, 6H), |
| | 1.61-1.47 (m, 3H), 1.35 |
| | (s, 3H), 1.27-1.11 (m, |
| | 7H), 1.05-0.88 (m, 2H), |
| | 0.79 (s, 3H). |
| 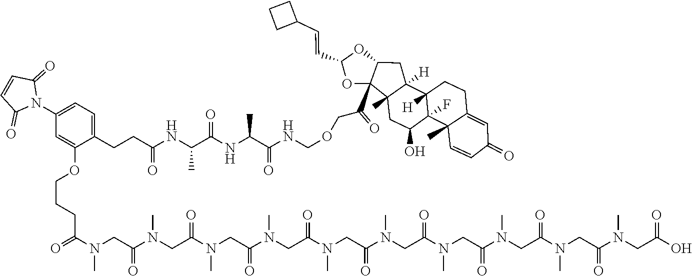 | MS m/z [M − H]⁻ (ESI): 1696.1; ¹H NMR (400 MHz, DMSO-d₆) δ7.33- 7.27 (m, 1H), 7.24-7.17 (m, 1H), 7.10 (s, 2H), 6.88 (d, J = 9.6 Hz, 1H), 6.83-6.72 (m, 1H), 6.28-6.20 (m, 1H), 6.09-6.00 (m, 2H), 5.27-5.17 (m, 1H), 4.99-4.89 (m, 1H), 4.82-4.74 (m, 1H), 4.67-4.44 (m, 3H), 4.32 (s, 4H), 4.28-4.15 (m, 10H), 4.14-3.90 (m, 12H), 3.03-2.72 (m, 34H), 2.6-2.58 (m, 1H), |
| | 2.45-2.29 (m, 4H), |
| | 2.07-1.89 (m, 6H), |
| | 1.88-1.51 (m, 9H), |
| | 1.50-1.46 (m, 3H), |
| | 1.41-1.29 (m, 1H), |
| | 1.27-1.15 (m, 6H), 0.82 |
| | (s, 3H). |

### Example 84. Preparation of 1-(2-((7S,10S)-1-((2S,6aS,6bR,7S,8aS,8bS,10R,11aR,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-7,10-dimethyl-1,6,9,12-tetraoxo-3-oxa-5,8,11-triazatetradecan-14-yl)-5-(2,5-dioxo-2,5-dihydro-1H-pyrrol-1-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid

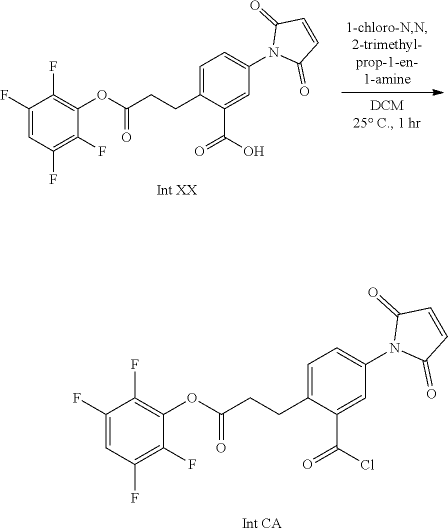 [SMILES: O=C(CCc1ccc(N2C(=O)C=CC2=O)cc1C(=O)O)Oc1c(F)c(F)cc(F)c1F ; O=C(CCc1ccc(N2C(=O)C=CC2=O)cc1C(=O)Cl)Oc1c(F)c(F)cc(F)c1F]

To a solution of 5-(2,5-dioxopyrrol-1-yl)-2-[3-oxo-3-(2,3,5,6-tetrafluorophenoxy)propyl] benzoic acid Int XX (1.80 g, 4.12 mmol, 1.0 eq) in DCM (36 mL) was added 1-chloro-N,N,2-trimethyl-prop-1-en-1-amine (1.10 g, 8.23 mmol, 1.09 mL, 2.0 eq). The mixture was stirred at 25° C. for 1 hr. The reaction mixture was concentrated under reduced pressure to remove solvent. Used for next step directly with no further purification. (2,3,5,6-tetrafluorophenyl) 3-[2-chlorocarbonyl-4-(2,5-dioxopyrrol-1-yl)phenyl]propanoate Int CA (1.88 g, 4.13 mmol, 100% yield) was obtained as yellow oil.

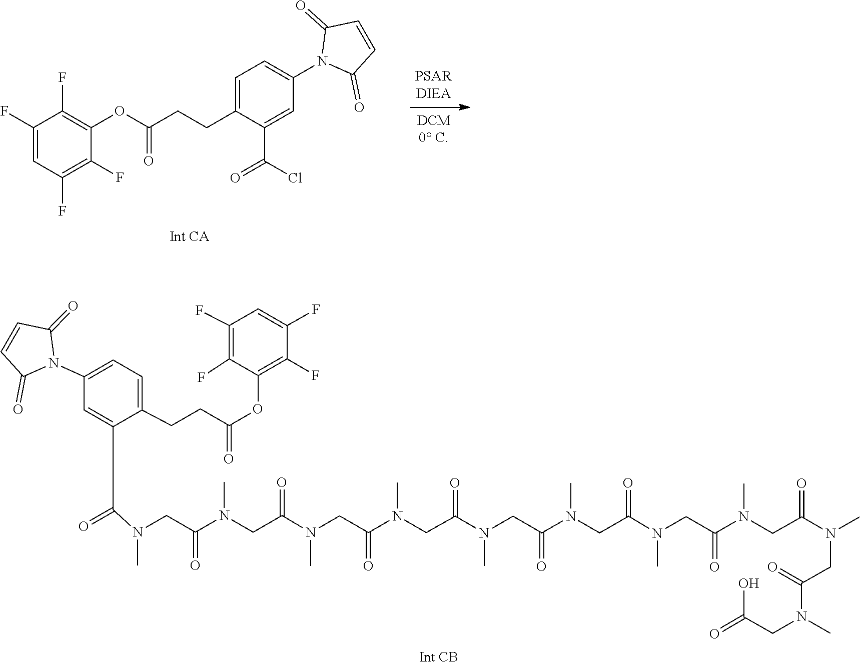 [SMILES: O=C(CCc1ccc(N2C(=O)C=CC2=O)cc1C(=O)Cl)Oc1c(F)c(F)cc(F)c1F ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)c1cc(N2C(=O)C=CC2=O)ccc1CCC(=O)Oc1c(F)c(F)cc(F)c1F]

To a solution of 2-[methyl-[2-[methyl-[2-[methyl-[2-[methyl-[2-[methyl-[2-[methyl-[2-[methyl-[2-[methyl-[2-[methyl-[2-(methylamino)acetyl]amino]acetyl]amino]acetyl]amino]-acetyl]amino]acetyl]amino]acetyl]amino]acetyl] amino]acetyl] amino]acetyl] amino]acetic acid (3.01 g, 4.13 mmol, 1.0 eq) PSAR in DCM (36 mL) was added DIEA (1.60 g, 12.4 mmol, 2.16 mL, 3.0 eq) at 0° C. After 10 min, to a solution of (2,3,5,6-tetrafluorophenyl) 3-[2-chlorocarbonyl-4-(2,5-dioxopyrrol-1-yl)phenyl]propanoate Int CA (1.88 g, 4.13 mmol, 1.0 eq) in DCM (18 mL) was added, and then stirred at 0° C. for 1 hr. The pH of the resulting mixture was adjusted to ˜6 with TFA at 0° C. and diluted with addition MeCN (2 mL) and concentrated under reduced pressure to remove DCM. The residue was purified by prep-HPLC (column: Phenomenex Luna C₁₈ (250×70 mm, 15 um); mobile phase: [water (TFA)-ACN]; B %: 25% 55%, 20 min). 2-[[2-[[2-[[2-[[2-[[2-[[2-[[2-[[2-[[2-[[5-(2,5-dioxopyrrol-1-yl)-2-[3- oxo-3-(2,3,5,6-tetrafluorophenoxy)propyl]benzoyl]-methyl-amino]acetyl]-methyl-amino]acetyl methyl-amino]acetyl]-methyl-amino]acetyl]-methyl-amino]acetyl]-methyl-amino]acetyl]methyl-amino]acetyl]-methyl-amino]acetyl]-methyl-amino]acetyl]-methyl-amino]acetic acid Int CB (360 mg, 314 μmol, 7.60% yield) was obtained as a white solid: LC/MS [M+H] 1148.4 (calculated); LC/MS [M+H] 1148.5 (observed).

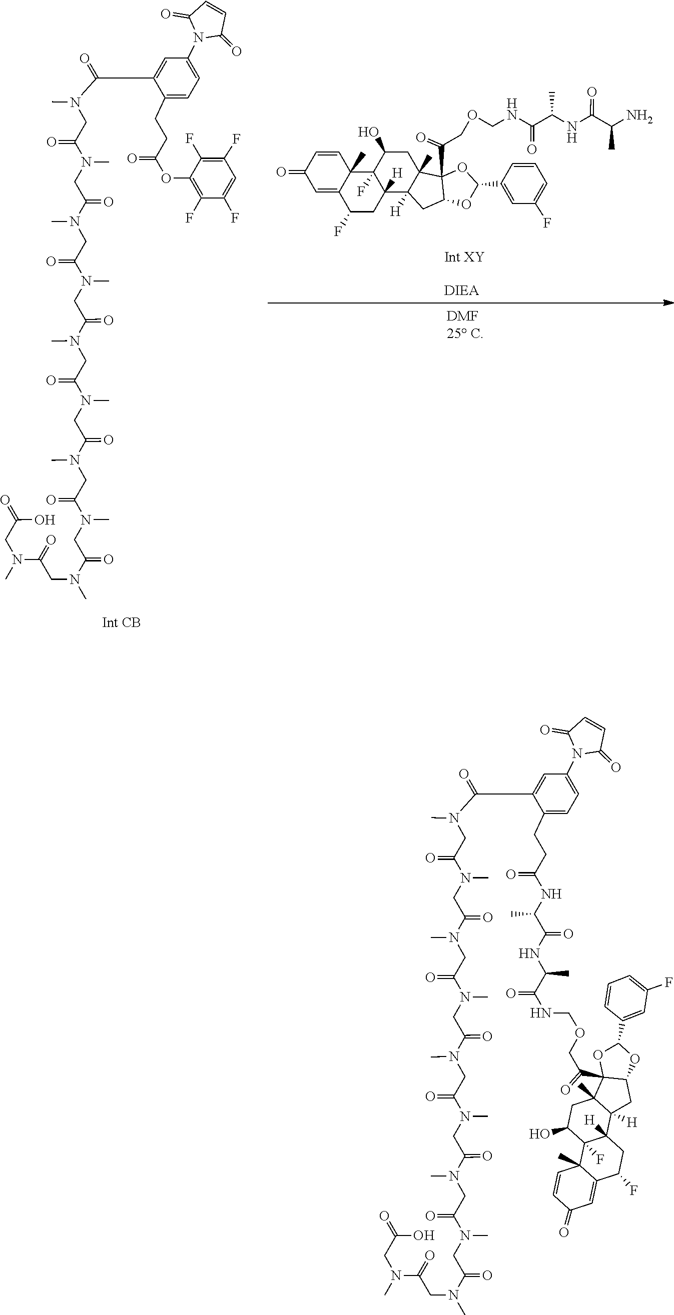

To a solution of (2S)-2-amino-N-[(1S)-2-[[2-[(1S,2S,4R,6R,8S,9S,11 S,12R,13 S,19S)-12,19-difluoro-6-(3-fluorophenyl)-11-hydroxy-9,13-dimethyl-16-oxo-5,7-dioxapentacyclo[10.8.0.02,9.04,8.013,18]icosa-14,17-dien-8-yl]-2-oxo-ethoxy]methylamino]-1-methyl-2-oxo-ethyl]propanamide Int CB (30.0 mg, 43.5 μmol, 1.0 eq) in DMF (0.5 mL) was added DIEA (16.8 mg, 130 μmol, 22.7 uL, 3.0 eq) and 2-[[2-[[2-[[2-[[2-[[2-[[2-[[2-[[2-[[2-[[5 (2,5-dioxopyrrol-1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methylamino]acetic acid (49.9 mg, 43.5 μmol, 1.0 eq), and then stirred at 25° C. for 1 hour. The pH of the mixture was adjusted ˜6 with TFA at 0° C. and diluted with addition MeCN (0.5 mL). The mixture was purified by (column: C₁₈-1 150×30 mm×5 um; mobile phase: [water(TFA)-ACN]; B %: 20%-45%, 20 min. The eluent was removed under freeze drying. 2-[[2-[[2-[[2-[[2-[[2-[[2-[[2-[[2-[[2-[[2-[3-[[(1S)-2-[[(1S)-2-[[2-[(1S,2S,4R,6R,8 S,9S,11 S,12R,13S,19S)-12,19-difluoro-6-(3-fluorophenyl)-11-hydroxy-9,13-dimethyl-16-oxo-5,7-dioxapentacyclo[10.8.0.02,9.04, 8.013,18]icosa-14,17-dien-8-yl]-2-oxo-ethoxy]methylamino]-1-methyl-2-oxo-ethyl]amino]-1-methyl-2-oxo-ethyl] amino]-3-oxo-propyl]-5-(2,5-dioxopyrrol-1-yl)benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetic acid (11.6 mg, 6.91 μmol, 15.89% yield, 99.61% purity) was obtained as a white solid: ¹H NMR (400 MHz, DMSO-d₆) δ 8.75-8.57 (m, 1H), 8.39-7.95 (m, 1H), 7.50-7.19 (m, 6H), 7.18-6.97 (m, 2H), 6.28 (d, J=10.4 Hz, 1H), 6.12 (s, 1H), 5.75-5.50 (m, 2H), 4.93 (d, J=4.0 Hz, 1H), 4.74-4.50 (m, 3H), 4.43-3.86 (m, 23H), 3.05-2.59 (m, 32H), 2.60-2.54 (m, 1H), 2.43 (s, 2H), 2.35-2.27 (m, 1H), 2.23-2.10 (m, 1H), 2.04-1.91 (m, 1H), 1.80-1.61 (m, 3H), 1.58-1.38 (m, 4H), 1.29-1.09 (m, 6H), 0.85 (s, 3H); LC/MS [M−H] 1669.7 (calculated); LC/MS [M−H] 1670.0 (observed).

### Example 85. Preparation of GR Agonist-Linker Compounds

The compounds in Table 16 below were prepared in a manner similar to that described Example 84 using the appropriate compounds as starting material.

TABLE 16
GR Agonist-Linker Compounds

| Structure | MS/¹H NMR |
|---|---|
| 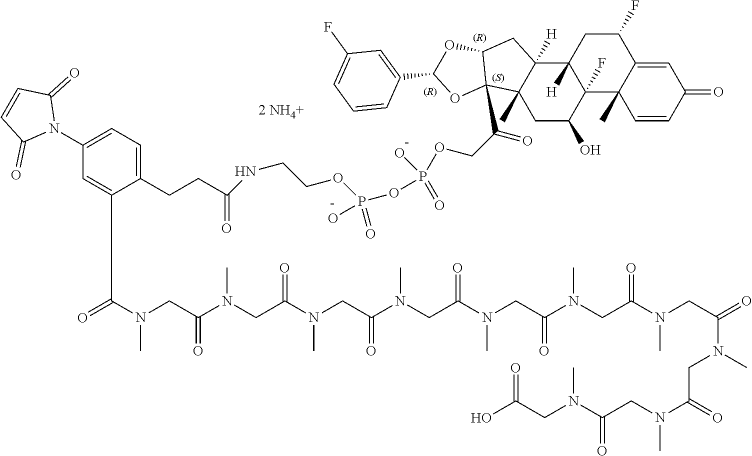 | MS m/z [M − H]⁻ (ESI): 1701.5896; ¹H NMR (DMSO-d₆, 400 MHz) δ 7.46-7.38 (m, 2H), 7.31- 7.14 (m, 6H), 7.08-7.01 (m, 2H), 6.26 (d, J = 9.6 Hz, 1H), 6.13 (s, 1H), 5.70-5.48 (m, 2H), 5.07- 4.92 (m, 2H), 4.73 (dd, J = 8.8 Hz, 18.0 Hz, 1H), 4.32-3.91 (m, 25H), 3.00-2.74 (m, 34H), 2.43 (t, J = 7.6 Hz, 2H), 2.25- 2.15 (m, 1H), 2.04 (d, J = 14.0 Hz, 1H), 1.89 (d, J = 14.0 Hz, 1H), 1.72 (d, J = 8.0 Hz, 2H), 1.50 (s, 4H), 0.92 (s, 3H) |
| 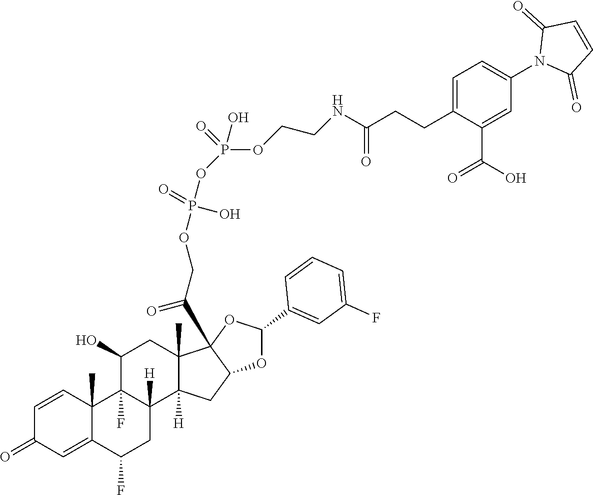 | MS m/z [M + H]⁺ (ESI): 993.4; ¹H NMR (DMSO-d₆, 400 MHz) δ 7.73 (d, J = 1.6 Hz, 1H), 7.46-7.38 (m, 3H), 7.31- 7.19 (m, 4H), 7.12 (s, 2H), 6.27 (dd, J = 1.6, 10.0 Hz, 1H), 6.12 (s, 1H), 5.74-5.52 (m, 2H), 5.05 (dd, J = 9.6, 18.8 Hz, 1H), 4.94 (d, J = 4.0 Hz, 1H), 4.74 (dd, J = 8.4, 18.8 Hz, 1H), 4.19 (d, J = 8.8 Hz, 1H), 3.91 (q, J = 6.0 Hz, 2H), 3.25 (t, J = 5.6 Hz, 2H), 3.13 (t, J = 8.0 Hz, 2H), 2.45- 2.38 (m, 2H), 2.31-2.26 (m, 1H), 2.20-2.12 (m, 1H), 2.02-1.92 (m, 1H), 1.82 (d, J = 14.0 Hz, 1H), 1.77-1.66 (m, 2H), 1.47 (s, 4H), 0.88 (s, 3H) |
| 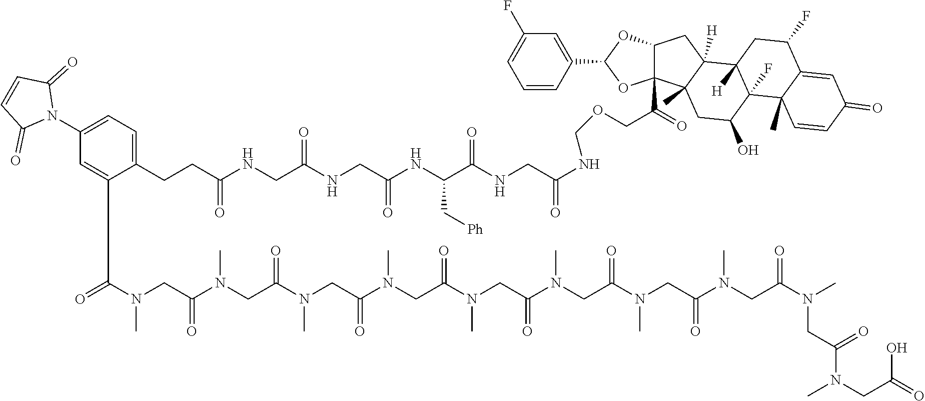 | MS m/z [M − H]⁻ (ESI): 1846.1; ¹H NMR (400 MHz, DMSO-d₆)δ 8.58 (s, 1H), 8.39-8.28 (m, 1H), 8.21-8.03 (m, 2H), 8.20-8.03 (m, 2H), 7.33- 7.07 (m, 11H), 6.25 (d, J = 9.6 Hz, 1H), 6.11 (s, 1H), 5.75-5.53 (m, 2H), 4.94 (d, J = 4.8 Hz, 1H), 4.72-4.56 (m, 3H), 4.50- 3.85 (m, 22H), 3.78-3.65 (m, 6H), 3.11-2.68 (m, 30H), 2.54-2.52 (m, 1H), 2.27-2.25 (m, 1H), 2.23- 2.10 (m, 2H), 1.99 (d, J = 12.8 Hz, 1H), 1.82- 1.63 (m, 3H), 1.48 (s, 4H), 0.86 (s, 3H). |
| 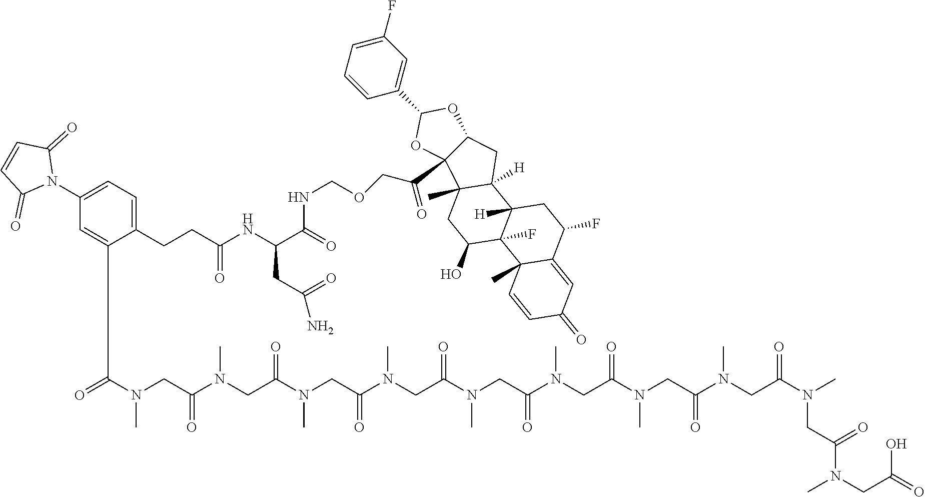 | MS m/z [M + Na]⁺ (ESI): 1665.8; ¹H NMR (400 MHz, DMSO-d₆) δ 8.72- 8.59 (m, 1H), 7.45-7.35 (m, 1H), 7.34-7.10 (m, 7H), 7.02 (s, 2H), 6.31- 6.23 (m, 1H), 6.13 (s, 1H), 5.69-5.60 (m, 1H), 5.57-5.52 (m, 1H), 4.89 (s, 1H), 4.63-4.50 (m, 4H), 4.32-4.15 (m, 12H), 2.98-2.67 (m, 35H), 2.44-2.28 (m, 5H), 2.18- 2.05 (m, 1H), 1.73-1.63 (m, 3H), 1.46-143 (m, 4H), 0.82 (s, 3H) |

### Example 86. Preparation of 1-(2-(3-((3-((2-(((2S,3R,4S,5S,6S)-6-carboxy-3,4,5-trihydroxytetrahydro-2H-pyran-2-yl)oxy)-5-(((((2-((2S,6aS,6bR,7S,8aS,8bS,10R,11aR,12aS,12bS)-2,6b-difluoro-10-(3-fluorophenyl)-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)methyl)(ethyl)carbamoyl)oxy)methyl)phenyl)amino)-3-oxopropyl)amino)-3-oxopropyl)-5-(2,5-dioxo-2,5-dihydro-1H-pyrrol-1-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid

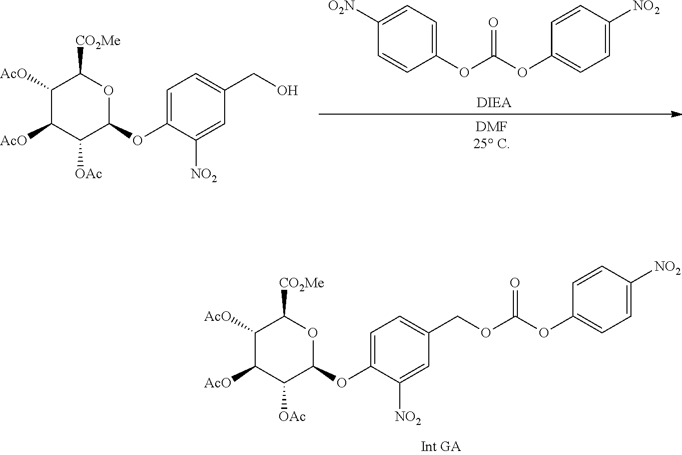 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; O=C(Oc1ccc([N+](=O)[O-])cc1)Oc1ccc([N+](=O)[O-])cc1 ; COC(=O)[C@H]1O[C@@H](Oc2ccc(COC(=O)Oc3ccc([N+](=O)[O-])cc3)cc2[N+](=O)[O-])[C@H](OC(C)=O)[C@@H](OC(C)=O)[C@@H]1OC(C)=O]

To a stirred solution of methyl (2S,3S,4S,5R,6S)-3,4,5-tris(acetyloxy)-6-[4-(hydroxymethyl)-2-nitrophenoxy]oxane-2-carboxylate (5.0 g, 10.3 mmol, 1.0 equiv.) and diisopropylethylamine (4.0 g, 31.0 mmol, 3.0 equiv.) in N,N-Dimethylformamide (50.0 mL) was added bis(4-nitrophenyl) carbonate (4.7 g, 15.5 mmol, 1.5 equiv.) at room temperature under air atmosphere. The resulting solution was stirred for 16 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 5.5 g (82%) of Int GA was obtained as a white solid. MS m/z [M+NH_{4]}⁺ (ESI): 668.12.

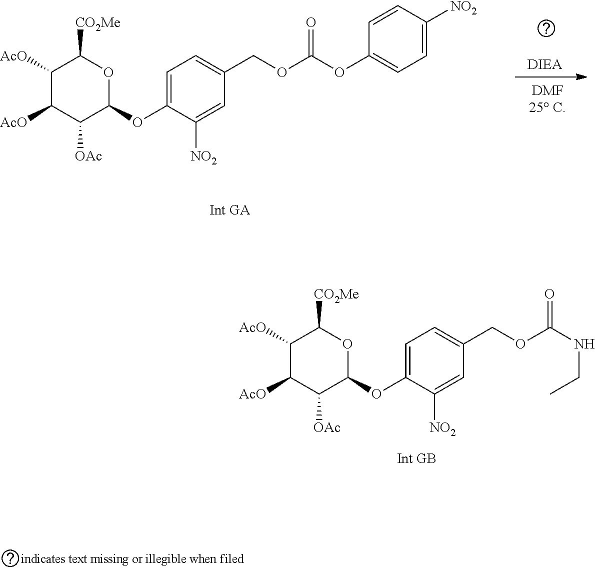 [SMILES: COC(=O)[C@H]1O[C@@H](Oc2ccc(COC(=O)Oc3ccc([N+](=O)[O-])cc3)cc2[N+](=O)[O-])[C@H](OC(C)=O)[C@@H](OC(C)=O)[C@@H]1OC(C)=O ; CCNC(=O)OCc1ccc(O[C@@H]2O[C@H](C(=O)OC)[C@@H](OC(C)=O)[C@H](OC(C)=O)[C@H]2OC(C)=O)c([N+](=O)[O-])c1]

To a stirred solution of Int GA (5.5 g, 8.5 mmol, 1.0 equiv.) and ethylamine (1.4 g, 16.9 mmol, 2.0 equiv.) in N,N-dimethylformamide (50.0 mL) was added diisopropylethylamine (3.3 g, 25.4 mmol, 3.0 equiv.) at 25° C. under air atmosphere. The resulting solution was stirred for 2 h at 25° C., diluted with water (500 mL). The resulting mixture was extracted with dichloromethane (3×500 mL) and organic layers were washed with saturated sodium chloride solution (3×500 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 3.5 g (75%) of Int GB was obtained as a white solid. MS m/z [M+NH₄]⁺ (ESI): 574.15.

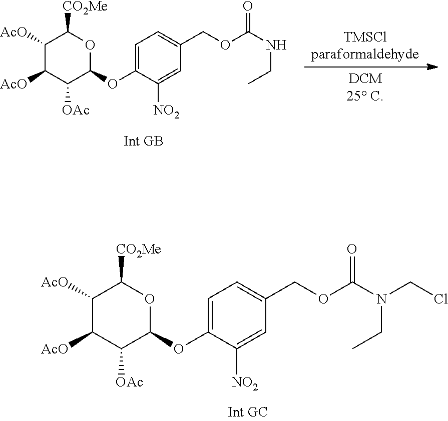 [SMILES: CCNC(=O)OCc1ccc(O[C@@H]2O[C@H](C(=O)OC)[C@@H](OC(C)=O)[C@H](OC(C)=O)[C@H]2OC(C)=O)c([N+](=O)[O-])c1 ; CCN(CCl)C(=O)OCc1ccc(O[C@@H]2O[C@H](C(=O)OC)[C@@H](OC(C)=O)[C@H](OC(C)=O)[C@H]2OC(C)=O)c([N+](=O)[O-])c1]

To a stirred solution of Int GB (3.5 g, 6.3 mmol, 1.0 equiv.) and paraformaldehyde (1.7 g, 18.9 mmol, 3.0 equiv.) in dichloromethane (35.0 mL) was added chlorotrimethylsilane (2.1 g, 18.9 mmol, 3.0 equiv.) at 25° C. under air atmosphere. The resulting mixture was stirred for additional 2 h at 25° C. The resulting mixture was concentrated under reduced pressure to afford Int GC (3.0 g, 79%) as a white solid.

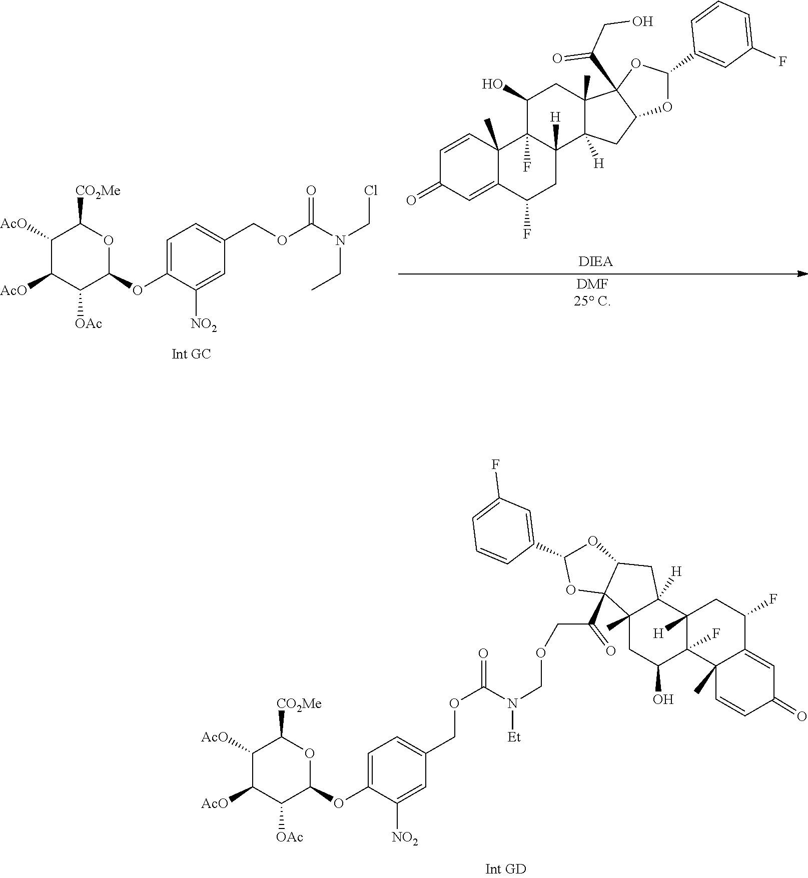

To a stirred solution of Int GC (3.0 g, 5.0 mmol, 2 equiv.) and diisopropylethylamine (51.28 mg, 0.397 mmol, 3 equiv.) in N,N-dimethylformamide (20.0 mL) was added starting material (1.3 g, 2.5 mmol, 1.0 equiv.) at 25° C. under air atmosphere. The resulting solution was stirred for 2 h at 25° C., diluted with water (300 mL). The resulting mixture was extracted with dichloromethane (3×300 mL) and organic layers were washed with saturated sodium chloride solution (3×300 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 1.1 g (41%) of Int GD was obtained as a white solid. MS m/z [M+H]⁺ (ESI): 1087.35.

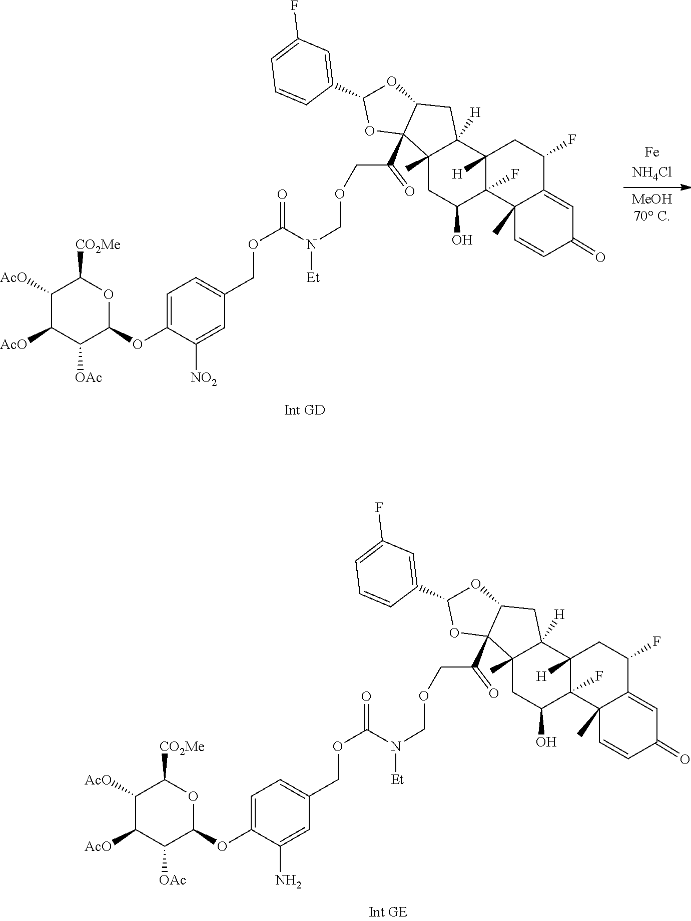

To a stirred solution methyl Int GD (1.1 g, 1.0 mmol, 1.0 equiv.) and ammonium chloride (536.8 mg, 10.1 mmol, 10.0 equiv.) in methanol (11.0 mL) was added iron powder (1.1 g, 20.0 mmol, 20.0 equiv.) at 25° C. The resulting mixture was stirred for 5 h at 70° C. The solids were filtered out and washed with methanol (3×10 mL). The resulting mixture was concentrated under reduced pressure. The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 810 mg (76%) of Int GE was obtained as a white solid. MS m/z [M+H]⁺ (ESI): 1057.37.

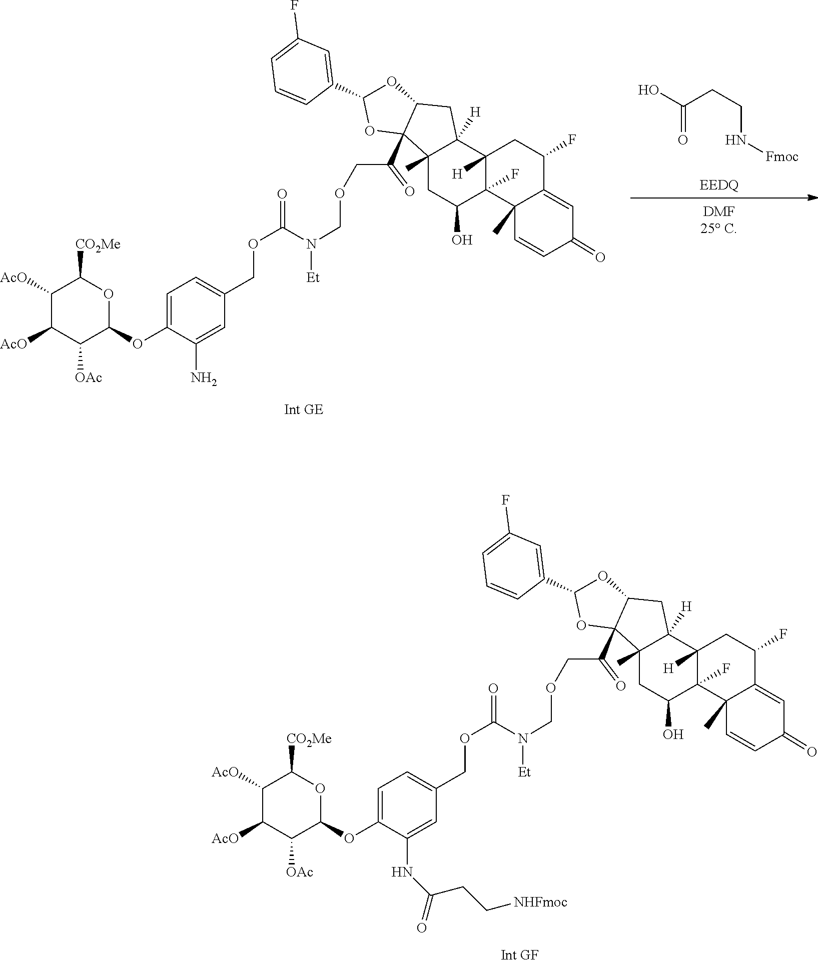

To a stirred solution Int GE (810.0 mg, 0.7 mmol, 1.0 equiv.) and ethyl 2-ethoxy-2H-quinoline-1-carboxylate (378.9 mg, 1.5 mmol, 2.0 equiv.) in dichloromethane (10.0 mL) was added 3-{[(9H-fluoren-9-ylmethoxy)carbonyl]amino}propanoic acid (357.8 mg, 1.2 mmol, 1.5 equiv.) at 25° C. The resulting solution was stirred for 3 h at 25° C., diluted with water (200 mL). The resulting mixture was extracted with dichloromethane (3×300 mL) and organic layers were washed with saturated sodium chloride solution (3×300 mL), dried over anhydrous sodium sulfate, filtration and concentrated under reduced pressure. The crude product was purified by Flash-Prep-HPLC with the following conditions (IntelFlash-1): Column, C₁₈ silica gel; mobile phase, water (10 mmol/L ammonium bicarbonate) and acetonitrile (10.0% acetonitrile up to 100.0% in 20 min); Detector, UV 254 nm. 600.0 mg (58%) of Int GF was obtained as a white solid. MS m/z [M+H]⁺ (ESI): 1050.48.

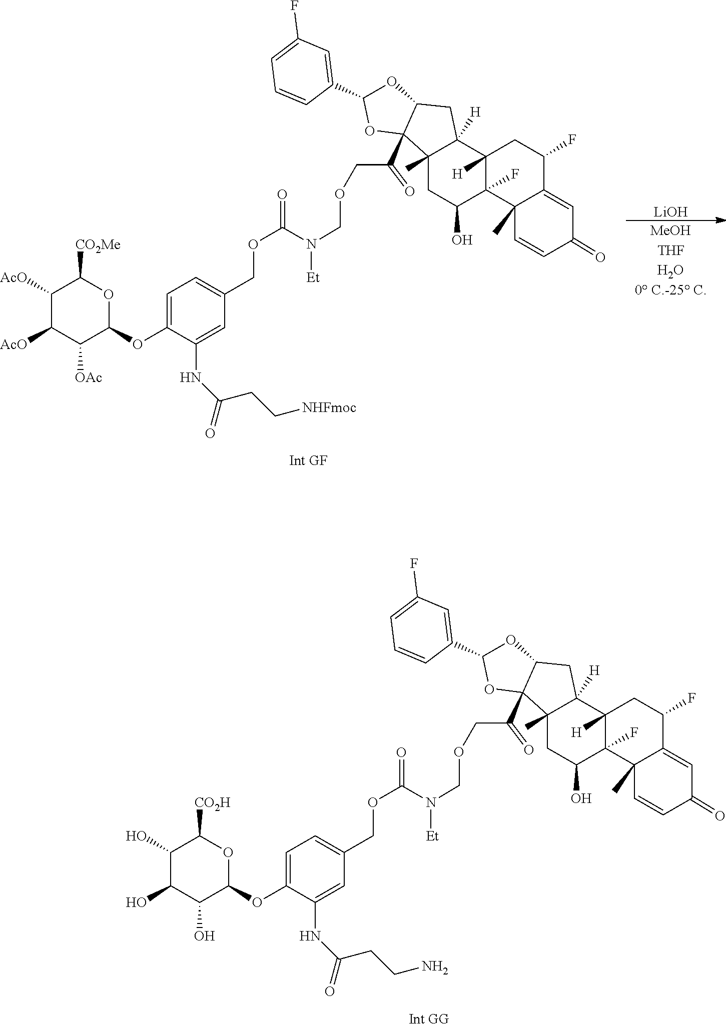

To a stirred solution of Int GF (600.0 mg, 0.4 mmol, 1.0 equiv.) in methanol (10.0 mL) and water (5.0 mL) with an inert atmosphere of nitrogen, was added lithium hydroxide (31.9 mg, 1.2 mmol, 3.0 equiv.) at 0° C. The resulting solution was stirred for 12 h at 25° C., adjusted pH to 7 with hydrochloric acid (1 mol/L). The crude product was purified by Flash-Prep-HPLC with the following conditions (IntelFlash-1): Column, C₁₈ silica gel; mobile phase, water and acetonitrile (10.0% acetonitrile up to 100.0% in 20 min); Detector, UV 254 nm. 180 mg (41%) of Int GG was obtained as a white solid. MS m/z [M+H]⁺ (ESI): 988.36.

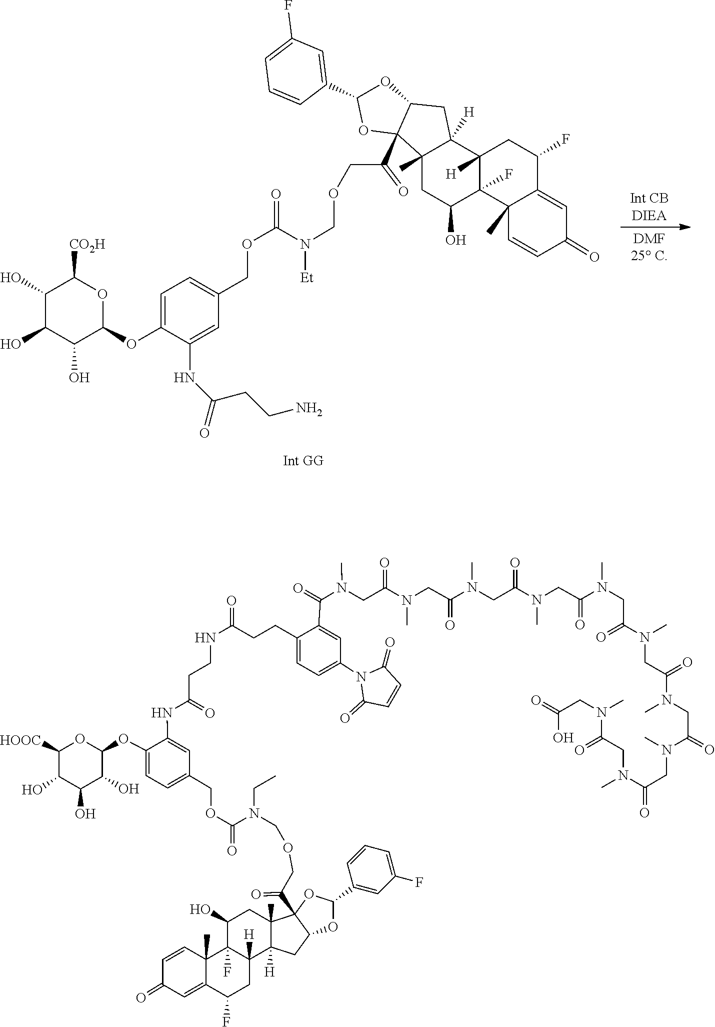

To a solution of (2S,3S,4S,5R,6S)-6-[2-(3-aminopropanoylamino)-4-[[[2-[(1S,2S,4R,6R,8S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04, 8.013,18]icosa-14,17-dien-8-yl]-2-oxo-ethoxy]methyl-ethyl-carbamoyl] oxymethyl]phenoxy]-3,4,5-trihydroxy-tetrahydropyran-2-carboxylic acid Int GG (30.0 mg, 30.3 μmol, 1.0 eq) in DMF (0.5 mL) was added DIEA (11.7 mg, 91.1 μmol, 15.8 uL, 3.0 eq) and 2-[[2-[[2-[[2-[[2-[[2-[[2-[[2-[[2-[[2-[[5-(2,5-dioxopyrrol- 1-yl)-2-[3-oxo-3-(2,3,5,6-tetrafluorophenoxy)propyl]benzoyl]-methyl-amino]acetyl]-methyl-amino]acetyl]-methyl-amino]acetyl]-methyl-amino]acetyl]-methyl-amino]acetyl]-methyl-amino]acetyl]-methyl-amino]acetyl]-methylamino]acetyl]-methyl-amino]acetyl]-methyl-amino]acetic acid (Int CB, 34.8 mg, 30.3 μmol, 1.0 eq.), and then stirred at 25° C. for 1 hour. The pH of the reaction mixture was adjusted to ˜6 with FA at 0° C. and diluted with addition MeCN (0.5 mL). The mixture was purified by prep-HPLC (column: Phenomenex Luna C₁₈ 200×40 mm×10 um; mobile phase: [water(FA)-ACN]; B %: 15%-50%, 8 min). The eluent was removed under freeze drying. (2S,3S,4S,5R,6S)-6-[2-[3-[3-[2-[[2-[[2-[[2-[[2-[[2-[[2-[[2-[[2-[[2-[carboxymethyl(methyl)amino]-2-oxo-ethyl]-methyl-amino]-2-oxo-ethyl]-methyl-amino]-2-oxo-ethyl]-methyl-amino]-2-oxo-ethyl]-methyl-amino]-2-oxo-ethyl]-methylamino]-2-oxo-ethyl]-methyl-amino]-2-oxo-ethyl]-methyl-amino]-2-oxo-ethyl]-methyl-amino]-2-oxo-ethyl]-methyl-carbamoyl]-4-(2,5-dioxopyrrol-1-yl)phenyl]propanoylamino]propanoylamino]-4-[[[2-[(1S,2S,4R,6R,8 S,9S,11S,12R,13S,19S)-12,19-difluoro-6-(3-fluorophenyl)-11-hydroxy-9,13-dimethyl-16-oxo-5,7-dioxapentacyclo[10.8.0.02,9.04, 8.013,18]icosa-14,17-dien-8-yl]-2-oxo-ethoxy]methyl-ethyl-carbamoyl] oxymethyl]phenoxy]-3,4,5-trihydroxy-tetrahydropyran-2-carboxylic acid (26.6 mg, 12.3 μmol, 40.77% yield, 91.69% purity) was obtained as a white solid: ¹H NMR (400 MHz, DMSO-d₆) δ 8.16 (s, 1H), 7.52-6.93 (m, 11H), 6.29 (dd, J=1.6, 10.0 Hz, 1H), 6.12 (s, 1H), 5.75-5.47 (m, 2H), 5.09-4.90 (m, 3H), 4.89-4.59 (m, 4H), 4.44-3.77 (m, 22H), 3.39-3.25 (m, 7H), 3.03-2.63 (m, 31H), 2.62-2.53 (m, 4H), 2.44-2.26 (m, 4H), 2.23-2.12 (m, 1H), 2.02-1.97 (m, 1H), 1.77-1.65 (m, 3H), 1.52-1.45 (m, 4H), 1.15-1.02 (m, 3H), 0.86 (s, 3H); LC/MS [M+H] 1969.8 (calculated); LC/MS [M+H] 1969.9 (observed).

### Example 87. Preparation of GR Agonist-Linker Compounds

The compounds in Table 17 below were prepared in a manner similar to that described Example 86 using the appropriate compounds as starting material.

TABLE 17
GR Agonist-Linker Compounds

| Structure | MS/¹H NMR |
|---|---|
| 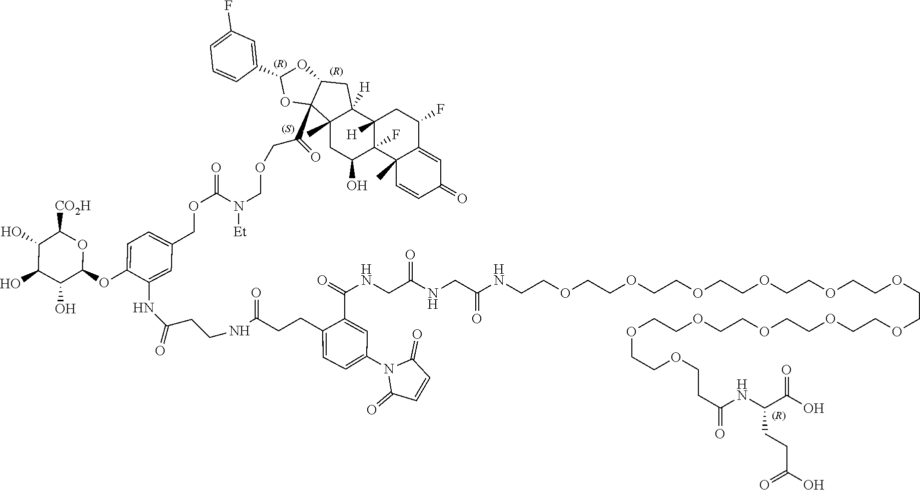 | MS m/z [M/2 − H]⁻ (ESI): 1050.05; ¹H NMR (300 MHz, Methanol-d₄) δ: 0.71- 0.93 (m, 4H), 1.08 (t, J = 7.1 Hz, 4H), 1.17- 1.31 (m, 1H), 1.47 (s, 4H), 1.67 (s, 3H), 1.76-1.95 (m, 2H), 2.00-2.17 (m, 2H), 2.19-2.33 (m, 4H), 2.32-2.55 (m, 6H), 3.04 (t, J = 7.3 Hz, 2H), 3.25-3.39 (m, 6H), 3.47-3.57 (m, 42H), 3.59-3.72 (m, 4H), 3.82 (s, 2H), 4.00 (s, 2H), 4.15- 4.37 (m, 4H), 4.63 (s, 2H), 4.76-5.07 (m, 6H), 5.41 (q, J = 23.0, 21.6 Hz, 3H), 6.24 (d, J = 19.0 Hz, 3H), 6.80 (s, 2H), 6.88-7.09 (m, 3H), 7.11-7.39 (m, 7H), 8.09 (s, 1H) |

### Example 88. Preparation of 34-(5-(2,5-dioxo-2,5-dihydro-1H-pyrrol-1-yl)-4-fluoro-2-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid (Compound Int H-9

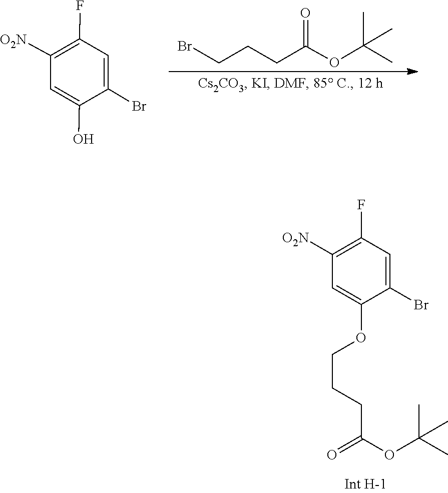 [SMILES: O=[N+]([O-])c1cc(O)c(Br)cc1F ; CC(C)(C)OC(=O)CCCBr ; CC(C)(C)OC(=O)CCCOc1cc([N+](=O)[O-])c(F)cc1Br]

To a mixture of 2-bromo-4-fluoro-5-nitrophenol (25.0 g, 106 mmol, 1.00 eq) and cesium carbonate (69.0 g, 212 mmol, 2.00 eq), potassium iodide (1.76 g, 10.6 mmol, 0.100 eq) in dimethyl formamide (200 mL) was added tert-butyl 4-bromobutanoate (35.5 g, 159 mmol, 1.50 eq). The mixture was stirred at 85° C. for 12 h. The reaction mixture was poured into ice water (500 mL). After filtration, the filter cake was dried under reduced pressure to afford tert-butyl 4-(2-bromo-4-fluoro-5-nitrophenoxy)butanoate, Int H-1, (40.0 g, crude) as a yellow solid.

¹H NMR (400 MHz, DMSO-d₆) δ=8.07 (d, J=10.4 Hz, 1H), 7.76 (d, J=6.4 Hz, 1H), 4.16 (t, J=6.4 Hz, 2H), 2.44-2.38 (m, 2H), 1.99-1.95 (m, 2H), 1.40 (s, 9H).

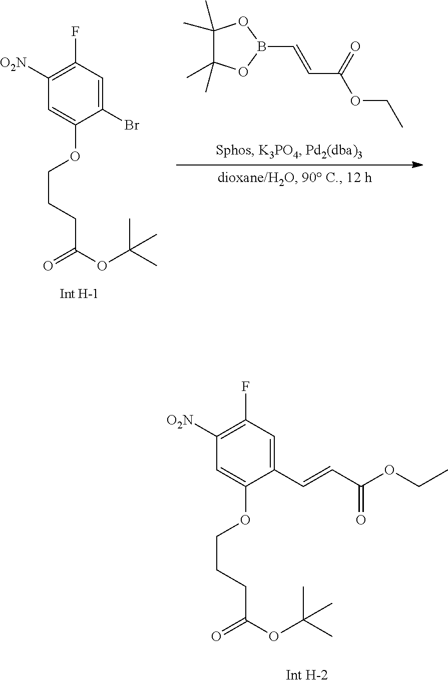 [SMILES: CC(C)(C)OC(=O)CCCOc1cc([N+](=O)[O-])c(F)cc1Br ; CCOC(=O)/C=C/B1OC(C)(C)C(C)(C)O1 ; CCOC(=O)/C=C/c1cc(F)c([N+](=O)[O-])cc1OCCCC(=O)OC(C)(C)C]

A mixture of tert-butyl 4-(2-bromo-4-fluoro-5-nitrophenoxy)butanoate (20.0 g, 52.9 mmol, 1.00 eq), ethyl (E)-3-(4,4,5,5-tetramethyl-1,3,2-dioxaborolan-2-yl)acrylate, Int H-1, (12.0 g, 52.9 mmol, 1.00 eq), tris(dibenzylideneacetone)dipalladium(0) (4.84 g, 5.29 mmol, 0.100 eq), dicyclohexyl(2′,6′-dimethoxy-[1,1′-biphenyl]-2-yl)phosphane (4.34 g, 10.6 mmol, 0.200 eq), potassium phosphate (22.5 g, 106 mmol, 2.00 eq) in water (40.0 mL) and dioxane (160 mL) was stirred at 90° C. for 12 h under nitrogen atmosphere. The mixture was concentrated to give a residue. The residue was purified by column chromatography (SiO₂, petroleum ether/ethyl acetate=1/0 to 10/1). The desired fraction was collected and concentrated under reduced pressure to afford tert-butyl (E)-4-(2-(3-ethoxy-3-oxoprop-1-en-1-yl)-4-fluoro-5-nitrophenoxy)butanoate, Int H-2, (16.6 g, 41.8 mmol, 79% yield) as a yellow solid.

¹H NMR (400 MHz, DMSO-d₆) δ=8.08 (d, J=12.0 Hz, 1H), 7.80 (d, J=16.0 Hz, 1H), 7.74 (d, J=6.4 Hz, 1H), 6.89 (d, J=16.4 Hz, 1H), 4.23-4.18 (m, 2H), 2.50 (br s, 2H), 2.43-2.34 (m, 2H), 2.03-1.94 (m, 2H), 1.39 (s, 9H), 1.26 (t, J=7.2 Hz, 3H).

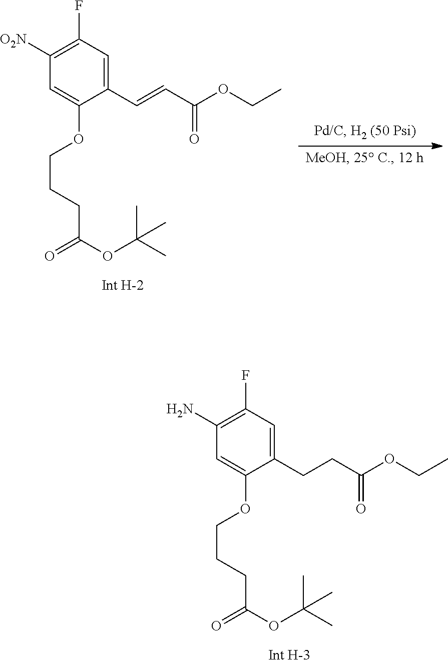 [SMILES: CCOC(=O)/C=C/c1cc(F)c([N+](=O)[O-])cc1OCCCC(=O)OC(C)(C)C ; CCOC(=O)CCc1cc(F)c(N)cc1OCCCC(=O)OC(C)(C)C]

To a solution of tert-butyl (E)-4-(2-(3-ethoxy-3-oxoprop-1-en-1-yl)-4-fluoro-5-nitrophenoxy)butanoate, Int H-2, (8.00 g, 20.1 mmol, 1.00 eq) in methanol (400 mL) was added palladium on activated carbon (1.00 g, 10% purity) under hydrogen (50 Psi). The mixture was stirred at 25° C. for 12 h. The mixture was filtered and the filtrate was concentrated to afford tert-butyl 4-(5-amino-2-(3-ethoxy-3-oxopropyl)-4-fluorophenoxy)butanoate, Int H-3, (6.86 g, crude) as yellow oil.

MS (ESI) m/z 370.3 [M+H]⁺

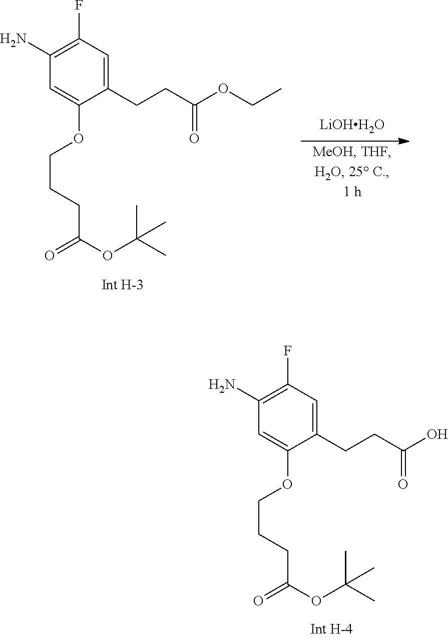 [SMILES: CCOC(=O)CCc1cc(F)c(N)cc1OCCCC(=O)OC(C)(C)C ; "LiOH·H2O" ; CC(C)(C)OC(=O)CCCOc1cc(N)c(F)cc1CCC(=O)O]

To a solution of tert-butyl 4-(5-amino-2-(3-ethoxy-3-oxopropyl)-4-fluorophenoxy)butanoate, Int H-3, (6.00 g, 16.2 mmol, 1.00 eq) in tetrahydrofuran (20.0 mL), methanol (20.0 mL) and water (20.0 mL) was added lithium hydroxide monohydrate (1.70 g, 40.6 mmol, 2.50 eq). The mixture was stirred at 25° C. for 1 h. The mixture was quenched with hydrochloric acid (1M) until pH=7. The mixture was concentrated to remove tetrahydrofuran and methanol. The mixture was diluted with water (200 mL) and extracted with ethyl acetate (3×100 mL). The organic layers was dried over anhydrous sodium sulfate and concentrated to afford 3-(4-amino-2-(4-(tert-butoxy)-4-oxobutoxy)-5-fluorophenyl)propanoic acid, Int H-4, (5.00 g, crude) as yellow oil.

¹H NMR (400 MHz, DMSO-d₆) δ=6.74 (d, J=12.0 Hz, 1H), 6.39-6.33 (m, 1H), 4.90 (br s, 2H), 3.88-3.78 (m, 2H), 2.68-2.53 (m, 2H), 2.40-2.37 (m, 2H), 1.99-1.89 (m, 4H), 1.39 (s, 9H).

MS (ESI) m/z 340.1 [M−H]⁻

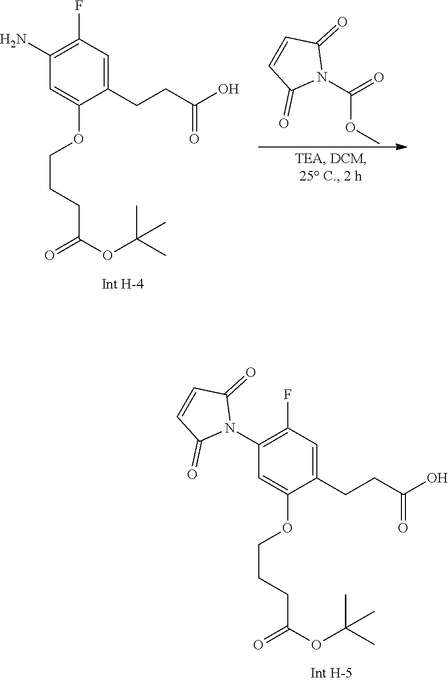 [SMILES: CC(C)(C)OC(=O)CCCOc1cc(N)c(F)cc1CCC(=O)O ; COC(=O)N1C(=O)C=CC1=O ; CC(C)(C)OC(=O)CCCOc1cc(N2C(=O)C=CC2=O)c(F)cc1CCC(=O)O]

To a solution of 3-(4-amino-2-(4-(tert-butoxy)-4-oxobutoxy)-5-fluorophenyl)propanoic acid, Int H-4, (2.78 g, 8.14 mmol, 1.00 eq) in dichloromethane (25.0 mL) were added triethylamine (2.47 g, 24.4 mmol, 3.40 mL, 3.00 eq) and methyl 2,5-dioxo-2,5-dihydro-1H-pyrrole-1-carboxylate (1.64 g, 10.6 mmol, 1.64 mL, 1.30 eq). The mixture was stirred at 25° C. for 2 h. The mixture was quenched with trifluoroacetic acid until pH=6, diluted with water (100 mL) and extracted with ethyl acetate (3×50 mL). The combined organic layers were dried over anhydrous sodium sulfate and concentrated to afford 3-(2-(4-(tert-butoxy)-4-oxobutoxy)-4-(2,5-dioxo-2,5-dihydro-1H-pyrrol-1-yl)-5-fluorophenyl)propanoic acid, Int H-5, (3.4 g, crude) as yellow oil.

MS (ESI) m/z 420.2 [M−H]⁻

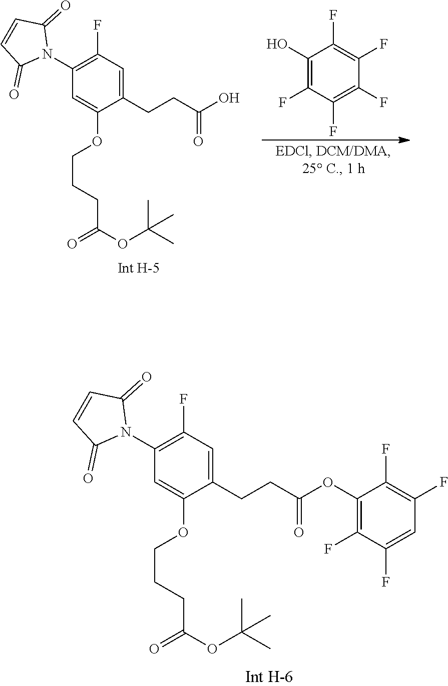 [SMILES: CC(C)(C)OC(=O)CCCOc1cc(N2C(=O)C=CC2=O)c(F)cc1CCC(=O)O ; Oc1c(F)c(F)c(F)c(F)c1F ; CC(C)(C)OC(=O)CCCOc1cc(N2C(=O)C=CC2=O)c(F)cc1CCC(=O)Oc1c(F)c(F)cc(F)c1F]

To a mixture of 3-(2-(4-(tert-butoxy)-4-oxobutoxy)-4-(2,5-dioxo-2,5-dihydro-1H-pyrrol-1-yl)-5-fluorophenyl)propanoic acid, Int H-5, (4.00 g, 9.49 mmol, 1.00 eq) and 2,3,5,6-tetrafluorophenol (3.15 g, 19.0 mmol, 2.00 eq) in N,N-dimethylacetamide (3.00 mL) and dichloromethane (30.0 mL) was added 3-(((ethylimino) methylene)amino)-N,N-dimethylpropan-1-aminium chloride (3.64 g, 19.0 mmol, 2.00 eq). The mixture was stirred at 25° C. for 1 h. The mixture was concentrated to give a residue. The residue was purified by column chromatography (SiO₂, petroleum ether/ethyl acetate=1/0 to 10/1). The desired fraction was collected and concentrated under reduced pressure to afford tert-butyl 4-(5-(2,5-dioxo-2,5-dihydro-1H-pyrrol-1-yl)-4-fluoro-2-(3-oxo-3-(2,3,5,6-tetrafluorophenoxy)propyl)phenoxy)butanoate, Int H-6, (500 mg, 878 μmol, 9% yield) as a yellow solid.

¹H NMR (400 MHz, DMSO-d₆) δ=7.98-7.89 (m, 1H), 7.32 (d, J=10.4 Hz, 1H), 7.25 (s, 2H), 7.07 (d, J=6.4 Hz, 1H), 3.98-3.93 (m, 2H), 3.15-3.09 (m, 2H), 3.04-2.98 (m, 2H), 2.39 (t, J=7.6 Hz, 2H), 1.98-1.92 (m, 2H), 1.36 (s, 9H).

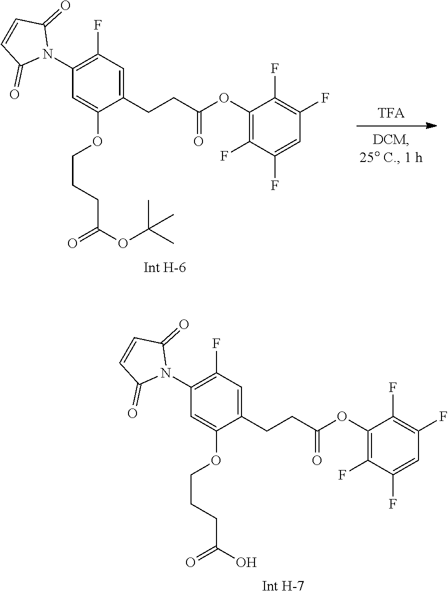 [SMILES: CC(C)(C)OC(=O)CCCOc1cc(N2C(=O)C=CC2=O)c(F)cc1CCC(=O)Oc1c(F)c(F)cc(F)c1F ; O=C(O)CCCOc1cc(N2C(=O)C=CC2=O)c(F)cc1CCC(=O)Oc1c(F)c(F)cc(F)c1F]

To solution of tert-butyl 4-(5-(2,5-dioxo-2,5-dihydro-1H-pyrrol-1-yl)-4-fluoro-2-(3-oxo-3-(2,3,5,6-tetrafluorophenoxy)propyl)phenoxy)butanoate, Int H-6, (500 mg, 878 μmol, 1.00 eq) in dichloromethane (12.0 mL) was added trifluoroacetic acid (3.00 mL). The mixture was stirred at 25° C. for 1 h. The mixture was concentrated under reduced pressure to give a residue. Then the residue was diluted with water (300 mL) and extracted with ethyl acetate (3×50.0 mL). The organic phases were washed with brine (20 mL), dried over anhydrous sodium sulfate and filtered. The filtrate was concentrated in vacuum, diluted with water (10 mL) and lyophilized to afford 4-(5-(2,5-dioxo-2,5-dihydro-1H-pyrrol-1-yl)-4-fluoro-2-(3-oxo-3-(2,3,5,6-tetrafluorophenoxy)propyl)phenoxy)butanoic acid, Int H-7, (450 mg, crude) as a white solid.

¹H NMR (400 MHz, DMSO-d₆) δ=12.24-12.06 (m, 1H), 7.94 (s, 1H), 7.31 (d, J=10.4 Hz, 1H), 7.25 (s, 2H), 7.08 (d, J=6.4 Hz, 1H), 3.96 (t, J=6.4 Hz, 2H), 3.17-3.09 (m, 2H), 3.05-2.95 (m, 2H), 2.41 (t, J=7.2 Hz, 2H), 1.96 (q, J=6.8 Hz, 2H).

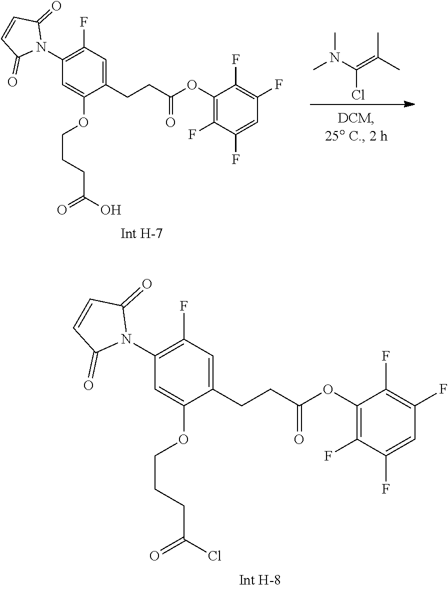

To a solution of 4-(5-(2,5-dioxo-2,5-dihydro-1H-pyrrol-1-yl)-4-fluoro-2-(3-oxo-3-(2,3,5,6-tetrafluorophenoxy)propyl)phenoxy)butanoic acid, Int H-7, (380 mg, 740 μmol, 1.00 eq) in dichloromethane (5.00 mL) was added 1-chloro-N,N,2-trimethylprop-1-en-1-amine (198 mg, 1.48 mmol, 196 μL, 2.00 eq). The mixture was stirred at 25° C. for 2 h. The mixture was concentrated under reduced pressure to afford 2,3,5,6-tetrafluorophenyl 3-(2-(4-chloro-4-oxobutoxy)-4-(2,5-dioxo-2,5-dihydro-1H-pyrrol-1-yl)-5-fluorophenyl) propanoate, Int H-8, (390 mg, crude) as yellow oil which was used in next step without purification.

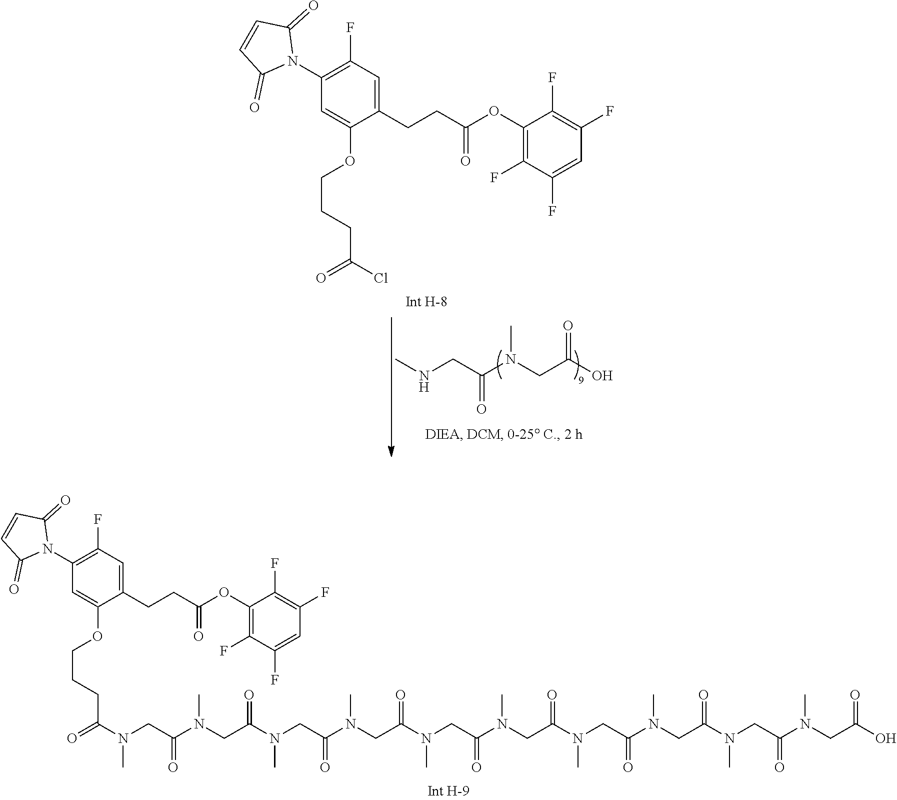

To a solution of 2,3,5,6-tetrafluorophenyl 3-(2-(4-chloro-4-oxobutoxy)-4-(2,5-dioxo 2,5-dihydro-1H-pyrrol-1-yl)-5-fluorophenyl)propanoate, Int H-8, (390 mg, 733 μmol, 1.00 eq) in dichloromethane (10.0 mL) was added diisopropylethylamine (284 mg, 2.20 mmol, 383 μL, 3.00 eq) and 5,8,11,14,17,20,23,26,29-nonamethyl-4,7,10,13,16,19,22,25,28-nonaoxo-2,5,8,11,14,17,20,23,26,29-decaazahentriacontan-31-oic acid (2.84 g, 3.89 mmol, 1.00 eq) at 0° C. The mixture was stirred at 25° C. for 2 h. The mixture was concentrated under reduced pressure to give a residue, which was purified by reverse-phase HPLC (0.1% formic acid condition). The desired fraction was collected and lyophilized to afford 34-(5-(2,5-dioxo-2,5-dihydro-1H-pyrrol-1-yl)-4-fluoro-2-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int H-9, (400 mg, 327 μmol, 44% yield, 98% purity) as a white solid.

¹H NMR (400 MHz, DMSO-d₆) δ=7.39-7.22 (m, 1H), 6.71-6.60 (m, 3H), 6.49-6.38 (m, 1H), 3.68-3.28 (m, 24H), 2.60-2.45 (m, 2H), 2.33-2.12 (m, 32H), 1.36-1.27 (m, 2H).

MS (ESI) m/z 1224.7 [M+2H]⁺

### Example 89. Preparation of 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid (Compound Int J-11)

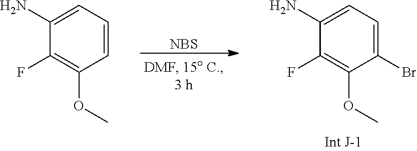

To a solution of 2-fluoro-3-methoxyaniline (11.0 g, 77.9 mmol, 1.00 eq) in N,N-dimethyl formamide (40.0 mL) was added a solution of 1-bromopyrrolidine-2,5-dione (13.9 g, 78.0 mmol, 1.00 eq) dropwise. The mixture was stirred at 15° C. for 3 h. The mixture was diluted with brine (300 mL) and extracted with ethyl acetate (3×300 mL). The organic layers were collected and dried over anhydrous sodium sulfate. After filtration, the filtrate was concentrated under reduced pressure to give a crude product, which was purified by column chromatography on silica gel (SiO₂, petroleum ether/ethyl acetate=1/0 to 3/1) and concentrated to afford 4-bromo-2-fluoro-3-methoxyaniline, Int J-1, (26.0 g, 118 mmol, 75% yield) as yellow oil.

¹H NMR (400 MHz, DMSO-d₆) δ=7.03 (dd, J=8.8, 1.2 Hz, 1H), 6.47 (t, J=8.8 Hz, 1H), 5.38 (s, 2H), 3.86-3.78 (m, 3H).

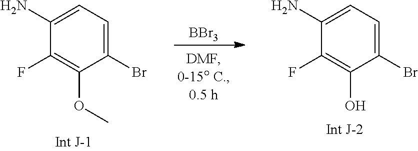

To a solution of 4-bromo-2-fluoro-3-methoxyaniline, Int J-1, (10.0 g, 45.5 mmol, 1.00 eq) in dichloromethane (100 mL) was added borontribromide (171 g, 682 mmol, 65.7 mL, 15.0 eq) at 0° C. The mixture was stirred at 15° C. for 0.5 h. The reaction mixture was quenched by addition water (300 mL) at 0° C. After filtration, the filtrate was extracted with ethyl acetate (3×200 mL). The organic layers were collected and dried over anhydrous sodium sulfate. After filtration, the filtrate was concentrated under reduced pressure to afford 3-amino-6-bromo-2-fluorophenol, Int J-2, (22.5 g, crude) as yellow oil.

¹H NMR (400 MHz, DMSO-d₆) δ=9.95-9.35 (m, 1H), 6.90 (dd, J=8.8, 1.6 Hz, 1H), 6.20-6.16 (m, 1H), 5.15 (br s, 2H).

MS (ESI) m/z 205.9 [M+H]⁺

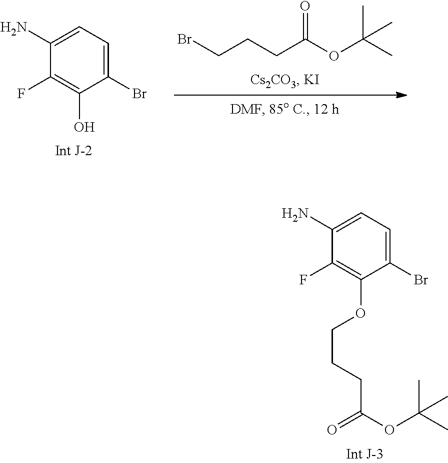 [SMILES: Nc1ccc(Br)c(O)c1F ; CC(C)(C)OC(=O)CCCBr ; CC(C)(C)OC(=O)CCCOc1c(Br)ccc(N)c1F]

To a solution of 3-amino-6-bromo-2-fluorophenol, Int J-2, (16.0 g, 77.7 mmol, 1.00 eq), cesium carbonate (50.6 g, 155 mmol, 2.00 eq) and potassium iodide (1.29 g, 7.77 mmol, 0.100 eq) in N, N-dimethyl formamide (50.0 mL) was added tert-butyl 4-bromobutanoate (19.1 g, 85.4 mmol, 1.10 eq). The mixture was stirred at 85° C. for 12 h. The mixture was diluted with brine (300 mL) and extracted with ethyl acetate (3×200 mL). The organic layers were collected and dried over anhydrous sodium sulfate. After filtration, the filtrate was concentrated under reduced pressure to give a crude product, which was purified by column chromatography on silica gel (SiO₂, petroleum ether/ethyl acetate=1/0 to 3/1) and concentrated to afford tert-butyl 4-(3-amino-6-bromo-2-fluorophenoxy)butanoate, Int J-3, (17.0 g, 48.8 mmol, 62% yield) as brown oil.

¹H NMR (400 MHz, DMSO-d₆) δ=7.03 (dd, J=8.8, 1.6 Hz, 1H), 6.46 (t, J=8.8 Hz, 1H), 5.36 (s, 2H), 3.98-3.93 (m, 2H), 2.43 (t, J=7.2 Hz, 2H), 1.91 (br t, J=6.8 Hz, 2H), 1.40 (s, 9H).

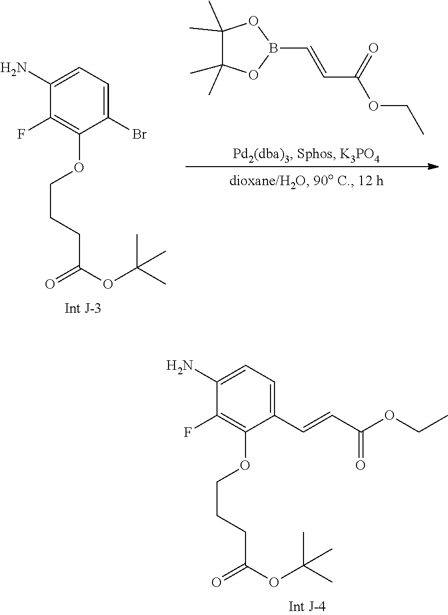 [SMILES: CC(C)(C)OC(=O)CCCOc1c(Br)ccc(N)c1F ; CCOC(=O)/C=C/B1OC(C)(C)C(C)(C)O1 ; CCOC(=O)/C=C/c1ccc(N)c(F)c1OCCCC(=O)OC(C)(C)C]

To a solution of tert-butyl 4-(3-amino-6-bromo-2-fluorophenoxy)butanoate, Int J-3, (12.0 g, 34.5 mmol, 1.00 eq) and ethyl (E)-3-(4,4,5,5-tetramethyl-1,3,2-dioxaborolan-2-yl)acrylate (19.5 g, 86.2 mmol, 2.50 eq) in dioxane (100 mL) and water (10.0 mL) was added 2-dicyclohexylphosphino-2,6-dimethoxybiphenyl (1.41 g, 3.45 mmol, 0.100 eq), potassium phosphate (11.0 g, 51.7 mmol, 1.50 eq) and bis(dibenzylideneacetone)-palladium(0) (1.58 g, 1.72 mmol, 0.0500 eq). The mixture was stirred at 90° C. for 12 h under nitrogen atmosphere. The mixture was concentrated under reduced pressure to give a crude product, which was purified by column chromatography on silica gel (SiO₂, petroleum ether/ethyl acetate=1/0 to 3/1) and concentrated to afford tert-butyl (E)-4-(3-amino-6-(3-ethoxy-3-oxoprop-1-en-1-yl)-2-fluorophenoxy)butanoate, Int J-4, (11.0 g, 30.0 mmol, 86% yield) as yellow solid.

¹H NMR (400 MHz, DMSO-d₆) δ=7.70 (d, J=16.0 Hz, 1H), 7.30 (dd, J=8.8, 1.2 Hz, 1H), 6.50 (t, J=8.4 Hz, 1H), 6.32 (d, J=16.0 Hz, 1H), 5.87 (s, 2H), 4.14 (q, J=7.2 Hz, 2H), 4.01 (t, J=6.4 Hz, 2H), 2.40 (t, J=7.2 Hz, 2H), 1.92 (quin, J=6.8 Hz, 2H), 1.38 (s, 9H), 1.25-1.22 (m, 3H).

### Example 90. Procedure for preparation of tert-butyl 4-(3-amino-6-(3-ethoxy-3-oxopropyl)-2-fluorophenoxy)butanoate

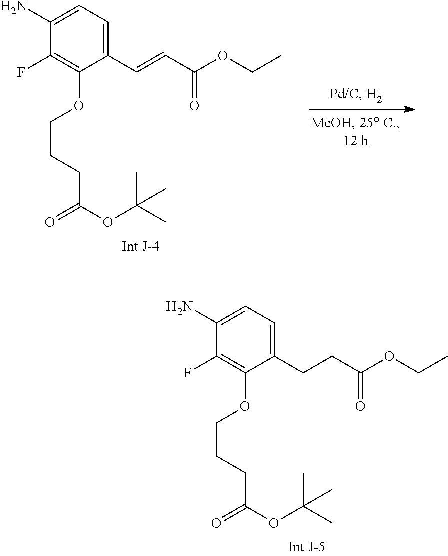 [SMILES: CCOC(=O)/C=C/c1ccc(N)c(F)c1OCCCC(=O)OC(C)(C)C ; CCOC(=O)CCc1ccc(N)c(F)c1OCCCC(=O)OC(C)(C)C]

A mixture of tert-butyl (E)-4-(3-amino-6-(3-ethoxy-3-oxoprop-1-en-1-yl)-2-fluorophenoxy)butanoate, Int J-4, (5.00 g, 13.6 mmol, 1.00 eq) in methanol (100 mL) was added palladium on activated carbon (5.00 g, 10% purity). The mixture was stirred at 25° C. for 12 h under hydrogen (50 Psi) atmosphere. The reaction mixture was filtered concentrated under reduced pressure to afford tert-butyl 4-(3-amino-6-(3-ethoxy-3-oxopropyl)-2-fluorophenoxy)butanoate, Int J-5, (3.40 g, crude) as black oil.

¹H NMR (400 MHz, DMSO-d₆) δ=6.64 (dd, J=8.4, 1.6 Hz, 1H), 6.39 (t, J=8.4 Hz, 1H), 5.01-4.91 (m, 2H), 4.03 (q, J=6.8 Hz, 2H), 3.93 (t, J=6.8 Hz, 2H), 2.74-2.64 (m, 2H), 2.47-2.44 (m, 2H), 2.39 (t, J=6.8 Hz, 2H), 1.90 (quin, J=6.8 Hz, 2H), 1.40 (s, 9H), 1.15 (t, J=6.8 Hz, 3H).

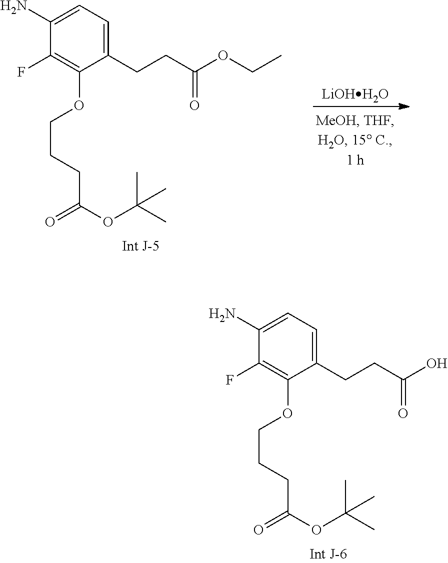 [SMILES: CCOC(=O)CCc1ccc(N)c(F)c1OCCCC(=O)OC(C)(C)C ; "LiOH·H2O" ; CC(C)(C)OC(=O)CCCOc1c(CCC(=O)O)ccc(N)c1F]

To a solution of tert-butyl 4-(3-amino-6-(3-ethoxy-3-oxopropyl)-2-fluorophenoxy)butanoate, Int J-5, (5.00 g, 13.5 mmol, 1.00 eq) in methanol (10.0 mL), tetrahydrofuran (10.0 mL) and water (10.0 mL) was added lithium hydroxide monohydrate (852 mg, 20.3 mmol, 1.50 eq). The mixture was stirred at 15° C. for 1 h. The mixture was quenched with hydrochloric acid (1M) until pH=7. The mixture was concentrated to remove tetrahydrofuran and methanol. The mixture was diluted with water (150 mL) and extracted with ethyl acetate (3×150 mL). The organic layers were collected and dried over anhydrous sodium sulfate. After filtration, the filtrate was concentrated under reduced pressure to afford 3-(4-amino-2-(4-(tert-butoxy)-4-oxobutoxy)-3-fluorophenyl)propanoic acid, Int J-6, (4.00 g, crude) as yellow oil.

¹H NMR (400 MHz, DMSO-d₆) δ=12.45-11.77 (m, 1H), 6.65 (dd, J=8.4, 1.2 Hz, 1H), 6.39 (t, J=8.4 Hz, 1H), 4.96 (s, 2H), 3.93 (t, J=6.4 Hz, 2H), 2.66 (t, J=7.2 Hz, 2H), 2.41-2.36 (m, 4H), 1.90 (quin, J=6.8 Hz, 2H), 1.40 (s, 9H).

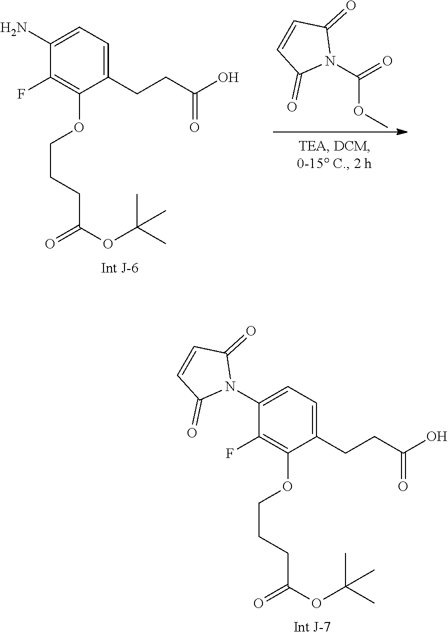 [SMILES: CC(C)(C)OC(=O)CCCOc1c(CCC(=O)O)ccc(N)c1F ; COC(=O)N1C(=O)C=CC1=O ; CC(C)(C)OC(=O)CCCOc1c(CCC(=O)O)ccc(N2C(=O)C=CC2=O)c1F]

To a solution of 3-(4-amino-2-(4-(tert-butoxy)-4-oxobutoxy)-3-fluorophenyl)propanoic acid, Int J-6, (4.00 g, 11.7 mmol, 1.00 eq) in dichloromethane (50.0 mL) was added triethylamine (3.56 g, 35.2 mmol, 4.89 mL, 3.00 eq) and methyl 2,5-dioxo-2,5-dihydro-1H-pyrrole-1-carboxylate (2.00 g, 12.9 mmol, 2.00 mL, 1.10 eq) at 0° C. The mixture was stirred at 15° C. for 2 h. The mixture was diluted with water (100 mL) and extracted with dichloromethane (3×80 mL). The organic layers were collected and dried over anhydrous sodium sulfate. After filtration, the filtrate was concentrated under reduced pressure to afford 3-(2-(4-(tert-butoxy)-4-oxobutoxy)-4-(2,5-dioxo-2,5-dihydro-1H-pyrrol-1-yl)-3-fluorophenyl)propanoic acid, Int J-7, (4.50 g, crude) as brown oil.

¹H NMR (400 MHz, DMSO-d₆) δ=7.25 (s, 2H), 7.16 (d, J=8.8 Hz, 1H), 7.08-7.03 (m, 1H), 4.01 (br t, J=6.4 Hz, 2H), 2.87 (br t, J=7.6 Hz, 2H), 2.52-2.51 (m, 2H), 2.42-2.36 (m, 4H), 1.39 (s, 9H).

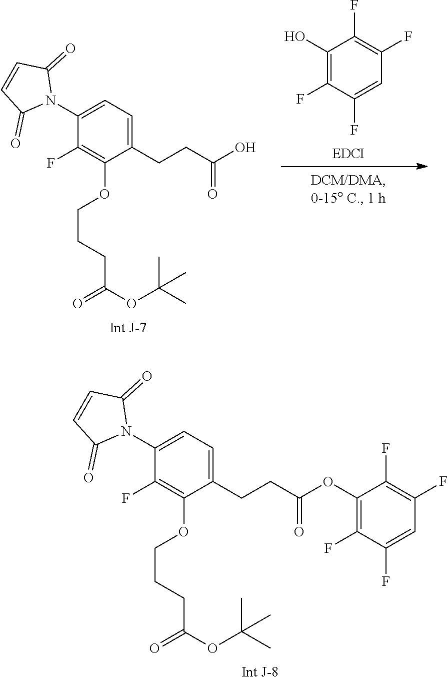 [SMILES: CC(C)(C)OC(=O)CCCOc1c(CCC(=O)O)ccc(N2C(=O)C=CC2=O)c1F ; Oc1c(F)c(F)cc(F)c1F ; CC(C)(C)OC(=O)CCCOc1c(CCC(=O)Oc2c(F)c(F)cc(F)c2F)ccc(N2C(=O)C=CC2=O)c1F]

To a solution of 3-(2-(4-(tert-butoxy)-4-oxobutoxy)-4-(2,5-dioxo-2,5-dihydro-1H-pyrrol-1-yl)-3-fluorophenyl)propanoic acid, Int J-7, (4.50 g, 10.7 mmol, 1.00 eq) and 2,3,5,6-tetrafluorophenol (3.55 g, 21.4 mmol, 2.00 eq) in dichloromethane (50.0 mL) and N,N-dimethylacetamide (5.00 mL) was added 1-(3-dimethylaminopropyl)-3-ethylcarbodiimide hydrochloride (4.09 g, 21.4 mmol, 2.00 eq) at 0° C. The mixture was stirred at 15° C. for 1 h. The mixture was concentrated under reduced pressure to give a crude product, which was purified by reversed-phase HPLC (column: spherical C18, 20-45 um, 100 Å, SW 330, mobile phase: [0.1% formic acid-acetonitrile]; B %: 85%-100%, 60 min). The desired fraction collected and lyophilized to afford tert-butyl 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoate, Int J-8, (3.00 g, 5.27 mmol, 49% yield) as a brown solid.

¹H NMR (400 MHz, DMSO-d₆) δ=7.97-7.91 (m, 1H), 7.26 (s, 2H), 7.24 (s, 1H), 7.13-7.08 (m, 1H), 4.06 (t, J=6.4 Hz, 2H), 3.18-3.12 (m, 2H), 3.10-3.04 (m, 2H), 2.43-2.39 (m, 2H), 1.97-1.92 (m, 2H), 1.37 (s, 9H).

MS (ESI) m/z 587.1 [M+H₂O]⁺

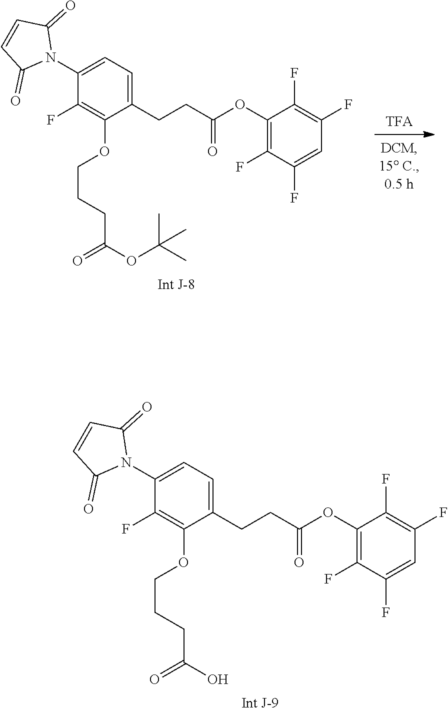 [SMILES: CC(C)(C)OC(=O)CCCOc1c(CCC(=O)Oc2c(F)c(F)cc(F)c2F)ccc(N2C(=O)C=CC2=O)c1F ; O=C(O)CCCOc1c(CCC(=O)Oc2c(F)c(F)cc(F)c2F)ccc(N2C(=O)C=CC2=O)c1F]

To a solution of tert-butyl 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoate, Int J-8, (1.00 g, 1.76 mmol, 1.00 eq) in dichloromethane (15.0 mL) was added trifluoroacetic acid (76.8 g, 673 mmol, 50.0 mL, 383 eq). The mixture was stirred at 15° C. for 0.5 h. The reaction mixture was lyophilized to give a crude product, which was triturated with methyl tert-butyl ether (10 mL) at 25° C. Then filtered, the filter cake was dried in vacuum to afford 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoic acid, Int J-9, (300 mg, 584 μmol, 33% yield) as a pink solid.

¹H NMR (400 MHz, DMSO-d₆) δ=13.15-11.06 (m, 1H), 7.94 (tt, J=7.6, 10.8 Hz, 1H), 7.26 (s, 2H), 7.24 (s, 1H), 7.15-7.06 (m, 1H), 4.08 (t, J=6.0 Hz, 2H), 3.23-3.13 (m, 2H), 3.11-3.02 (m, 2H), 2.42 (t, J=7.4 Hz, 2H), 1.96 (quin, J=6.8 Hz, 2H).

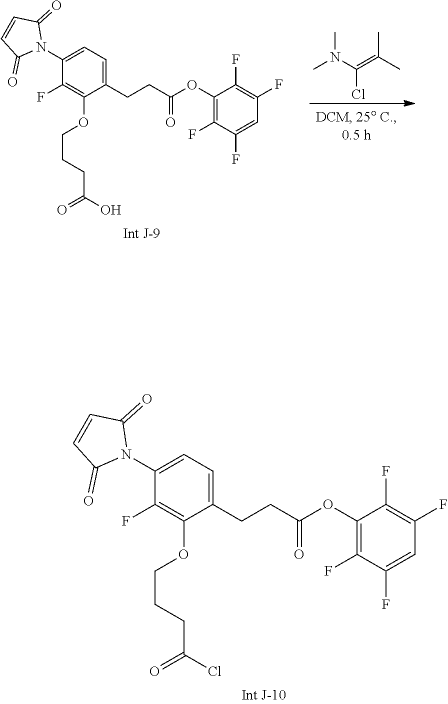

To a solution of 4-(3-(2,5-dioxo-2,5-dihydro-1H-pyrrol-1-yl)-2-fluoro-6-(3-oxo-3-(2,3,5,6-tetrafluorophenoxy)propyl)phenoxy)butanoic acid, Int J-9, (100 mg, 195 μmol, 1.00 eq) in dichloromethane (3.00 mL) was added 1-chloro-N,N,2-trimethylprop-1-en-1-amine, Int J-10, (52.1 mg, 390 μmol, 51.5 μL, 2.00 eq). The mixture was stirred at 25° C. for 0.5 h. The reaction mixture was used in next step directly.

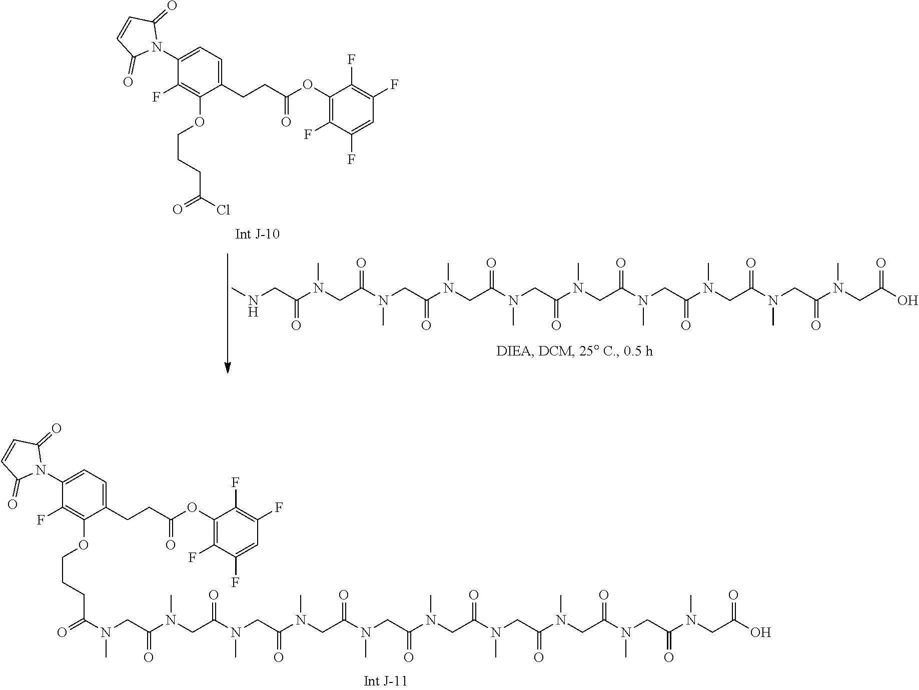 [SMILES: O=C(Cl)CCCOc1c(CCC(=O)Oc2c(F)c(F)cc(F)c2F)ccc(N2C(=O)C=CC2=O)c1F ; CNCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCCOc1c(CCC(=O)Oc2c(F)c(F)cc(F)c2F)ccc(N2C(=O)C=CC2=O)c1F]

To a solution of 5,8,11,14,17,20,23,26,29-nonamethyl-4,7,10,13,16,19,22,25,28-nonaoxo-2,5,8,11,14,17,20,23,26,29-decaazahentriacontan-31-oic acid, Intl-10, (137 mg, 188 μmol, 1.00 eq) in dichloromethane (2.00 mL) and N,N-diisopropylethylamine (72.9 mg, 564 μmol, 98.3 μL, 3.00 eq). The mixture was stirred at 25° C. for 10 min. Then the mixture was added 2,3,5,6-tetrafluorophenyl 3-(2-(4-chloro-4-oxobutoxy)-4-(2,5-dioxo-2,5-dihydro-1H-pyrrol-1-yl)-3-fluorophenyl)propanoate (100 mg, 188 μcool, 1.00 eq) at 0° C. The mixture was stirred at 25° C. for 20 min. The reaction mixture was concentrated under reduced pressure to give a crude product, which was purified by prep-HPLC (column: UniSil 3-100 C₁₈ Ultra (150×25 mm×3 um); mobile phase: [water (formic acid)-acetonitrile]; gradient: 20%-50% B over 40 min). The desired fraction collected and lyophilized to afford 34-(3-(2,5-dioxo-2,5-dihydro-1H-pyrrol-1-yl)-2-fluoro-6-(3-oxo-3-(2,3,5,6-tetrafluorophenoxy)propyl)phenoxy)-3,6,9,12,15,18,21,24,27,30-decamethyl-4,7,10,13,16,19,22,25,28,31-decaoxo-3,6,9,12,15,18,21,24,27,30-decaazatetratriacontanoic acid, Int J-11, (30.0 mg, 24.3 μmol, 12% yield, 99% purity) as a white solid.

¹H NMR (400 MHz, DMSO-d₆) δ=13.48-12.07 (m, 1H), 8.00-7.87 (m, 1H), 7.26 (s, 2H), 7.25-7.20 (m, 1H), 7.13-7.06 (m, 1H), 4.34-4.19 (m, 8H), 4.15-3.98 (m, 11H), 3.93 (br d, J=4.4 Hz, 2H), 3.16 (br d, J=6.0 Hz, 2H), 3.11-3.06 (m, 2H), 2.95-2.71 (m, 31H), 2.35-2.29 (m, 2H), 1.99-1.89 (m, 2H).

MS (ESI) m/z 1246.4 [M+Na]⁺

### Example 91. Preparation of GR Agonist-Linker Compounds

The compounds in Table 18 below were prepared in a manner similar to that described Example 83 using the appropriate compounds as starting material.

TABLE 18
GR Agonist-Linker Compounds

| Structure | |
|---|---|
| Compound No. | MS/¹H NMR |
| 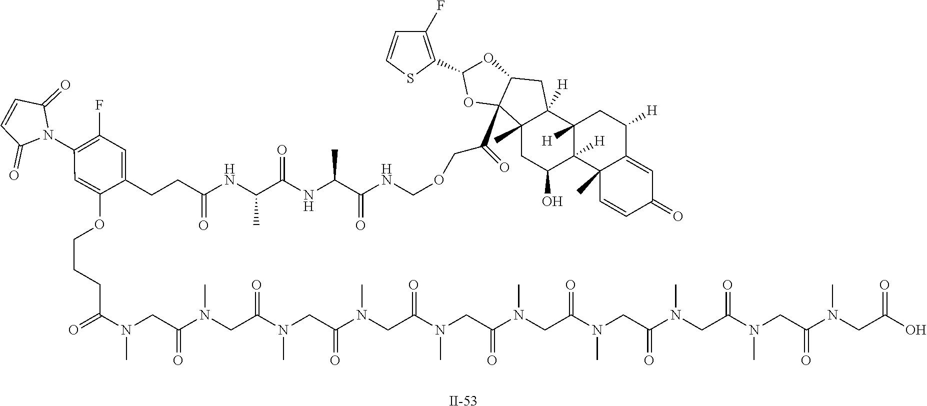 | ¹H NMR (400 MHz, DMSO-d₆) δ = 8.77-8.64 (m, 1H), 8.34-8.08 (m, 1H), 8.04 (d, J = 7.2 Hz, 1H), 7.62-7.55 (m, 1H), 7.33 (dd, J = 2.0, 10.0 Hz, 1H), 7.24 (s, 2H), 7.19-7.10 (m, 1H), 7.05- 6.92 (m, 2H), 6.19-6.16 (m, 1H), 6.04-5.91 (m, 2H), 4.92-4.85 (m, 1H), 4.84-4.72 (m, 1H), 4.70- 4.52 (m, 3H), 4.39-4.16 (m, 16H), 4.15-3.98 (m, 10H), 3.00-2.73 (m, 34H), 2.47-2.28 (m, 2H), 2.27-1.85 (m, 4H), 1.85- 1.60 (m, 5H), 1.40 (s, 3H), 1.29-1.10(m, 6H), 1.06-0.89 (m, 2H), 0.86 (s, 3H). MS (ESI) m/z 1717.8 [M + H]⁺ |
| 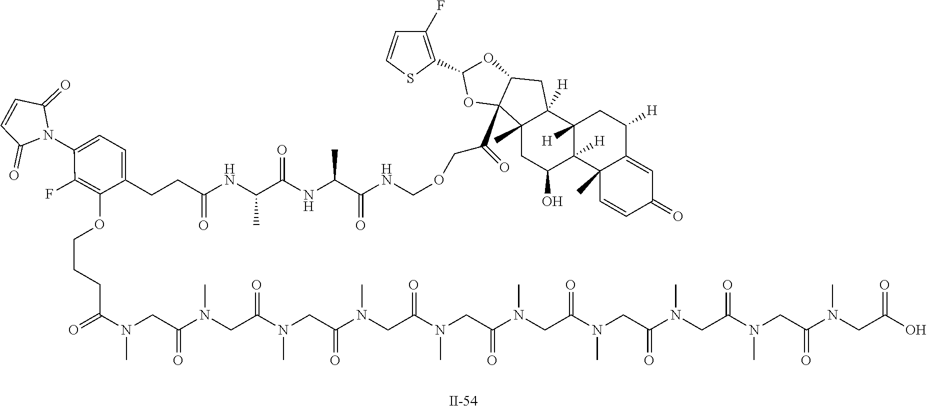 | ¹H NMR (400 MHz, DMSO-d₆) δ = 8.76-8.61 (m, 1H), 8.37-8.11 (m, 1H), 8.09-7.98 (m, 1H), 7.57 (br d, J = 4.0 Hz, 1H), 7.38-7.28 (m, 1H), 7.25 (s, 2H), 7.14-6.90 (m, 3H), 6.22-6.11 (m, 1H), 6.03-5.90 (m, 2H), 4.92-4.84 (m, 1H), 4.81- 4.64 (m, 2H), 4.62-4.48 (m, 2H), 4.41-3.90 (m, 24H), 3.08-2.62 (m, 34H), 2.37-1.59 (m, 12H), 1.39 (s, 3H), 1.26- 1.11 (m, 6H), 1.05-0.85 (m, 2H), 0.85 (s, 3H). MS (ESI) m/z 1717.70 [M + H]⁺ |
| 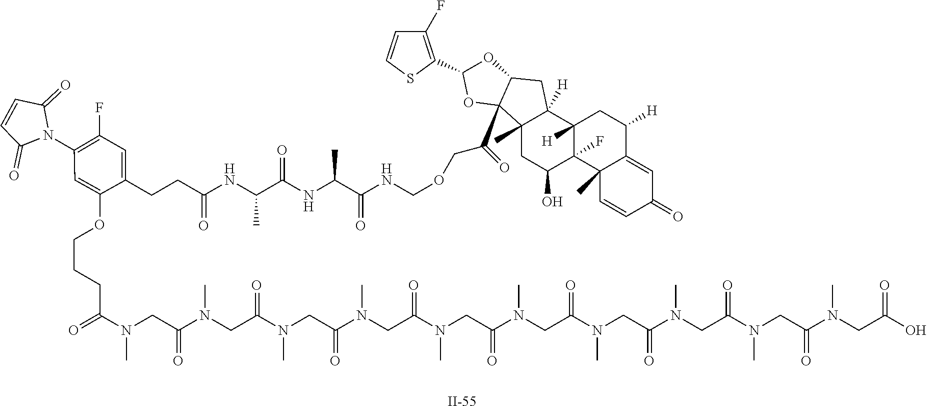 | ¹H NMR (400 MHz, DMSO-d₆) δ = 8.76-8.65 (m, 1H), 8.32-8.10 (m, 1H), 8.08-8.00 (m, 1H), 7.63-7.57 (m, 1H), 7.30 (dd, J = 3.2, 10.4 Hz, 1H), 7.24 (s, 2H), 7.15 (dd, J = 5.6, 10.4 Hz, 1H), 7.04-6.92 (m, 2H), 6.27-6.21 (m, 1H), 6.03 (d, J = 4.0 Hz, 1H), 5.42 (s, 1H), 4.89 (d, J = 3.2 Hz, 1H), 4.73-4.52 (m, 3H), 4.42-3.95 (m, 24H), 2.98-2.71 (m, 34H), 2.39-2.28 (m, 1H), 2.21- 2.11 (m, 1H), 2.08-1.79 (m, 4H), 1.73-1.70 (m, 1H), 1.63 (d, J = 8.4 Hz, 2H), 1.50 (s, 3H), 1.44- 1.30 (m, 1H), 1.22 (d, J = 7.2 Hz, 3H), 1.16 (d, J = 7.2 Hz, 3H), 0.86 (s, 3H). MS (ESI) m/z 1734.7 [M + Na]⁺ |
| 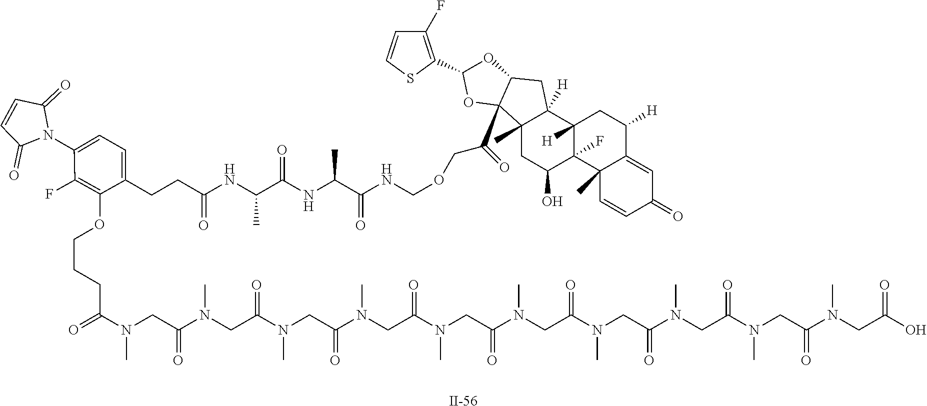 | ¹H NMR (400 MHz, DMSO-d₆) δ = 8.76-8.59 (m, 1H), 8.35-8.11 (m, 1H), 8.10-7.97 (m, 1H), 7.68-7.51 (m, 1H), 7.37- 7.20 (m, 3H), 7.18-6.86 (m, 3H), 6.30-6.17 (m, 1H), 6.03 (d, J = 5.0 Hz, 2H), 5.42 (br s, 1H), 4.95-4.82 (m, 1H), 4.79- 4.49 (m, 3H), 4.44-3.87 (m, 24H), 3.12-2.60 (m, 34H), 2.39-2.00 (m, 5H), 1.98-1.79 (m, 3H), 1.78- 1.57 (m, 3H), 1.49 (s, 3H), 1.43-1.30 (m, 1H), 1.28-1.07 (m, 6H), 0.86 (s, 3H). MS (ESI) m/z 1735.70 [M + H]⁺ |
| 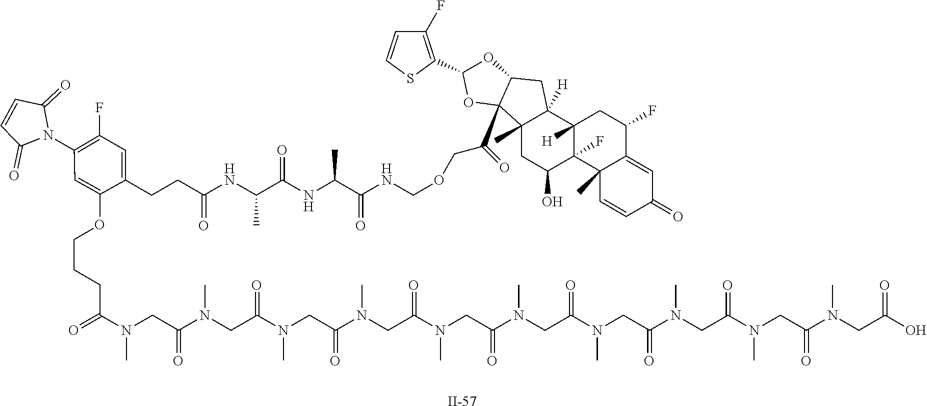 | ¹H NMR (400 MHz, DMSO-d₆) δ = 8.76-8.64 (m, 1H), 8.33-8.09 (m, 1H), 8.07-7.99 (m, 1H), 7.62-7.57 (m, 1H), 7.31- 7.21 (m, 3H), 7.17-7.09 (m, 1H), 7.04-6.90 (m, 2H), 6.34-6.27 (m, 1H), 6.12 (s, 1H), 6.03 (s, 1H), 5.76-5.55 (m, 1H), 5.52 (d, J = 2.0 Hz, 1H), 4.90 (s, 1H), 4.71-4.52 (m, 3H), 4.41-3.65 (m, 26H), 2.95-2.67 (m, 34H), 2.36-2.16 (m, 2H), 2.08-1.86 (m, 3H), 1.77- 1.63 (m, 3H), 1.53-1.42 (m, 4H), 1.21 (d, J = 7.2 Hz, 3H), 1.19-1.11 (m, 3H), 0.85 (s, 3H). MS (ESI) m/z 1775.6 [M + Na]⁺ |
| 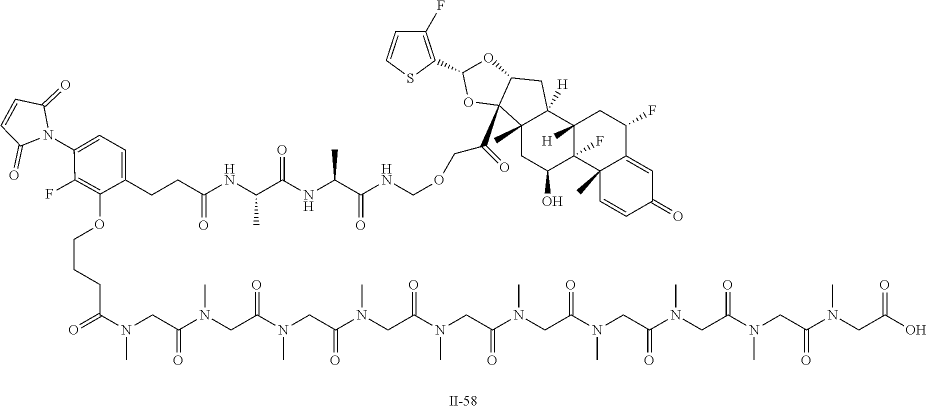 | ¹H NMR (400 MHz, DMSO-d₆) δ = 8.81-8.59 (m, 1H), 8.36-8.12 (m, 1H), 8.11-7.96 (m, 1H), 7.65-7.55 (m, 1H), 7.38- 7.22 (m, 3H), 7.15-6.99 (m, 2H), 6.98-6.92 (m, 1H), 6.30 (d, J = 10.7 Hz, 1H), 6.12 (s, 1H), 6.03 (s, 1H), 5.78-5.55 (m, 1H), 5.54-5.44 (m, 1H), 4.97-4.84 (m, 1H), 4.73-4.49 (m, 3H), 4.42- 3.85 (m, 24H), 3.09-2.59 (m, 34H), 2.36-2.16 (m, 5H), 2.11-1.85 (m, 3H), 1.80-1.59 (m, 3H), 1.57- 1.39 (m, 4H), 1.30-1.07 (m, 6H), 0.85 (s, 3H). MS (ESI) m/z 1753.70 [M + H]⁺ |
| 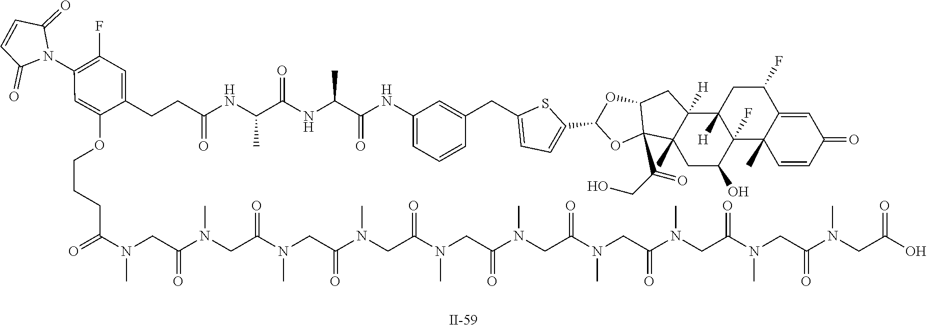 | ¹H NMR (400 MHz, DMSO-d₆) δ = 9.86 (s, 1H), 8.12-8.02 (m, 2H), 7.50-7.42 (m, 2H), 7.30- 7.21 (m, 3H), 7.20-7.11 (m, 2H), 7.09-7.04 (m, 1H), 7.03-6.95 (m, 1H), 6.90 (d, J = 7.2 Hz, 1H), 6.71 (d, J = 3.2 Hz, 1H), 6.34-6.26 (m, 1H), 6.14 (s, 1H), 5.80-5.74 (m, 1H), 5.73-5.55 (m, 1H), 5.54-5.43 (m, 1H), 5.00-4.88 (m, 1H), 4.50 (d, J = 19.2 Hz, 1H) 4.41-3.86 (m, 26H), 3.07-2.61 (m, 34H), 2.48-2.39 (m, 1H), 2.37- 2.13 (m, 2H), 2.06-1.82 (m, 3H), 1.80-1.59 (m, |
| | 3H), 1.56-1.39 (m, 3H), |
| | 1.27 (d, J = 7.2 Hz, 3H), |
| | 1.17 (d, J = 7.2 Hz, 3H), |
| | 0.82 (s, 3H). MS (ESI) |
| | m/z 1833.8 [M + Na]⁺ |
| 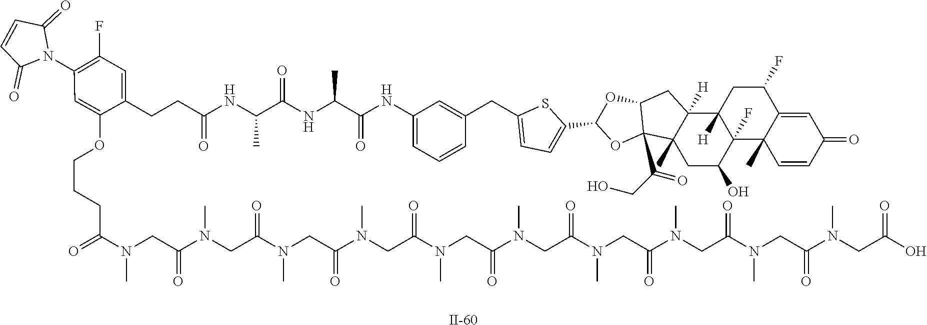 | |
| 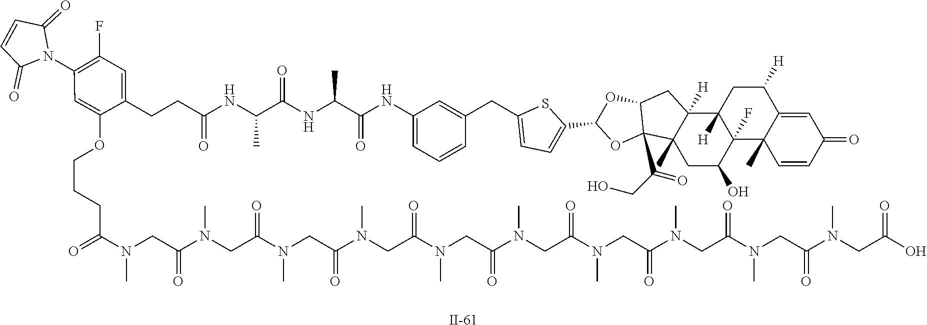 | ¹H NMR (400 MHz, DMSO-d₆) δ = 9.87 (s, 1H), 8.18-8.10 (m, 2H), 7.53-7.42 (m, 2H), 7.31- 7.26 (m, 1H), 7.24 (s, 2H), 7.22-7.12 (m, 2H), 7.07 (d, J = 4.0 Hz, 1H), 7.01 (t, J = 6.8 Hz, 1H), 6.91 (d, J = 7.2 Hz, 1H), 6.72 (d, J = 3.2 Hz, 1H), 6.24 (dd, J = 1.6, 10.0 Hz, 1H), 6.06 (s, 1H), 5.79-5.72 (m, 1H), 5.45- 5.36 (m, 1H), 4.98-4.87 (m, 1H), 4.50 (d, J = 19.2 Hz, 1H), 4.43-3.85 (m, 28H), 3.09-2.69 (m, 34H), 2.65-2.58 (m, 1H), 2.45-2.36 (m, 3H), 2.21- 2.07 (m, 1H), 2.05-1.73 |
| | (m, 5H), 1.68-1.56 (m, |
| | 3H), 1.49 (s, 3H), 1.43- |
| | 1.31 (m, 1H), 1.28 (d, |
| | J = 7.2 Hz, 3H), 1.18 (d, |
| | J = 6.8 Hz, 3H), 0.84 (s, |
| | 2H). MS (ESI) m/z |
| | 1793.8 [M + H]⁺ |
| 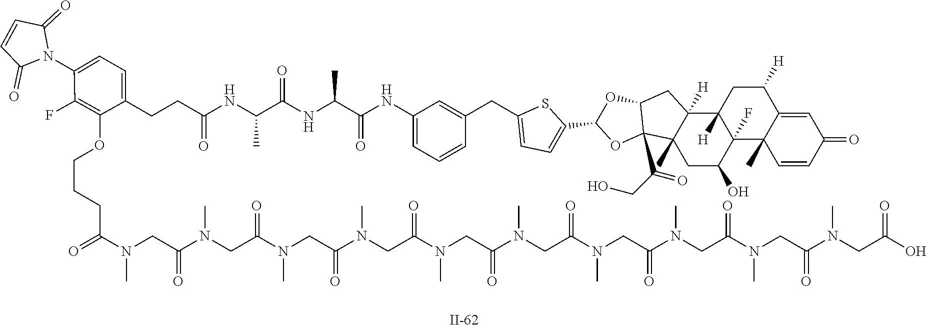 | ¹H NMR (400 MHz, DMSO-d₆) δ = 9.90-9.80 (m, 1H), 8.10 (d, J = 6.8 Hz, 2H), 7.51-7.42 (m, 2H), 7.29 (d, J = 10.4 Hz, 1H), 7.24 (s, 2H), 7.19 (t, J = 7.6 Hz, 1H), 7.14-6.98 (m, 3H), 6.90 (d, J = 7.2 Hz, 1H), 6.71 (d, J = 2.8 Hz, 1H), 6.23 (d, J = 10.0 Hz, 1H), 6.05 (s, 1H), 5.78-5.71 (m, 1H), 5.45-5.40 (m, 1H), 5.48-5.30 (m, 1H), 4.97-4.86 (m, 1H), 4.54- 4.43 (m, 1H), 4.37-4.00 (m, 24H), 3.95-3.90 (m, 2H), 2.98-2.71 (m, 34H), 2.46-2.28 (m, 1H), 2.27- 2.08 (m, 1H), 2.04-1.97 |
| | (m, 1H), 1.96-1.87 (m, |
| | 2H), 1.86-1.72 (m, 2H), |
| | 1.69-1.55 (m, 3H), 1.50 |
| | (s, 3H), 1.43-1.31 (m, |
| | 2H), 1.27 (d, J = 7.2 Hz, |
| | 3H), 1.18 (d, J = 7.2 Hz, |
| | 3H), 0.83 (s, 3H). MS |
| | (ESI) m/z 1793.8 |
| | [M + H]⁺ |
| 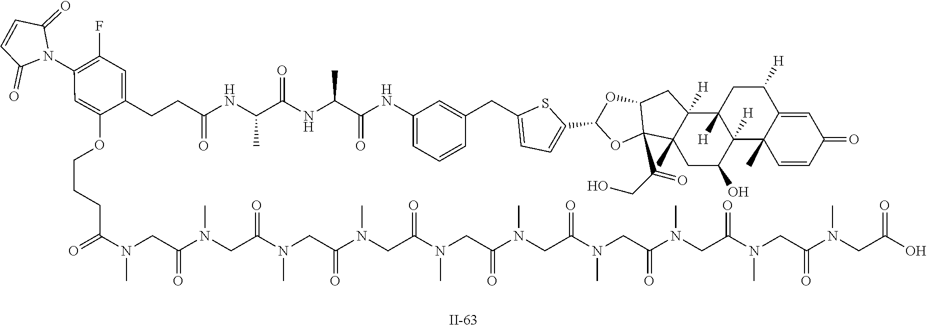 | ¹H NMR (400 MHz, DMSO-d₆) δ = 9.86 (s, 1H), 8.16-8.03 (m, 2H), 7.47 (s, 2H), 7.33-7.27 (m, 1H), 7.23 (s, 2H), 7.20-7.12 (m, 2H), 7.07 (d, J = 3.6 Hz, 1H), 7.00 (t, J = 7.2 Hz, 1H), 6.90 (d, J = 7.2 Hz, 1H), 6.72 (d, J = 3.2 Hz, 1H), 6.20- 6.14 (m, 1H), 5.97 (s, 1H), 5.77-5.72 (m, 1H), 4.96-4.82 (m, 2H), 4.52- 4.45 (m, 1H), 4.41-3.83 (m, 26H), 3.04-2.69 (m, 34H), 2.26-1.84 (m, 7H), 1.76-1.56 (m, 5H), 1.41- 1.36 (m, 3H), 1.27 (d, J = 7.2 Hz, 3H), 1.18 (d, J = 7.2 Hz, 3H), 0.83 (s, |
| | 3H). MS (ESI) m/z |
| | 1775.7[M + H]⁺ |
| 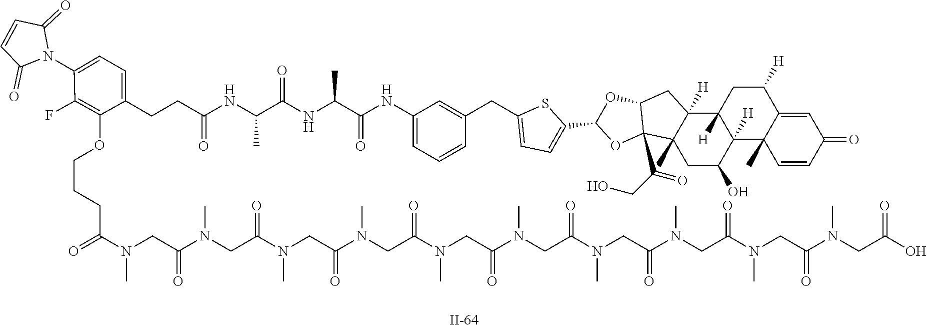 | ¹H NMR (400 MHz, DMSO-d₆) δ = 9.85 (s, 1H), 8.10 (d, J = 6.4 Hz, 2H), 7.47 (s, 2H), 7.31 (d, J = 10.0 Hz, 1H), 7.24 (s, 2H), 7.17 (t, J = 8.0 Hz, 1H), 7.14-7.09 (m, 1H), 7.08-6.99 (m, 2H), 6.90 (d, J = 7.2 Hz, 1H), 6.72 (d, J = 2.4 Hz, 1H), 6.18 (d, J = 9.6 Hz, 1H), 5.97 (s, 1H), 5.81- 5.67 (m, 1H), 4.97-4.85 (m, 1H), 4.83-4.70 (m, 1H), 4.49 (d, J = 19.6 Hz, 1H), 4.38-3.98 (m, 24H), 3.96-3.85 (m, 3H), 3.05-2.64 (m, 34H), 2.45-2.19 (m, 1H), 2.16- 2.07 (m, 1H), 2.06-1.99 (m, 1H), 1.97-1.77 (m, |
| | 3H), 1.75-1.49 (m, 5H), |
| | 1.39 (s, 3H), 1.28 (d, J = |
| | 6.8 Hz, 3H), 1.18 (d, J = |
| | 6.4 Hz, 3H), 1.02-0.85 |
| | (m, 2H), 0.83 (s, 3H). |
| | MS (ESI) m/z 1775.8 |
| | [M + H]⁺ |
| 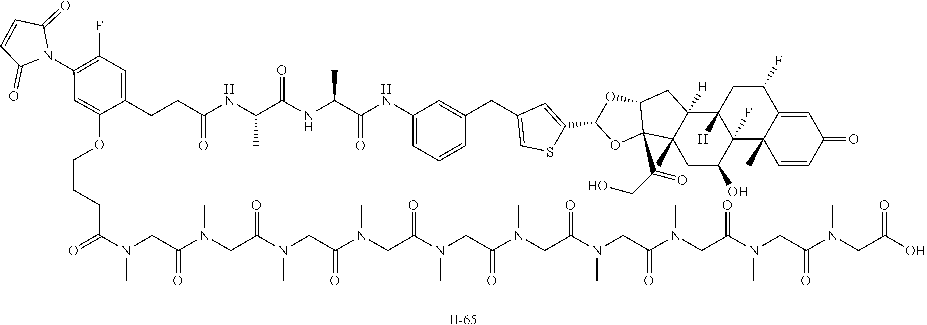 | ¹H NMR (400 MHz, DMSO-d₆) δ = 9.84 (s, 1H), 8.15-8.01 (m, 1H), 7.47-7.41 (m, 1H), 7.30- 7.11 (m, 5H), 7.05-6.96 (m, 2H), 6.88 (d, J = 7.2 Hz, 1H), 6.29 (dd, J = 1.6, 10.0 Hz, 1H), 6.13 (s, 1H), 5.80 (s, 1H), 5.74-5.54 (m, 1H), 5.50 (d, J = 3.2 Hz, 1H), 4.89 (s, 1H), 4.49 (d, J = 19.2 Hz, 1H), 4.42-3.84 (m, 26H), 3.81 (s, 2H), 3.04- 2.68 (m, 32H), 2.30-2.18 (m, 2H), 2.05-1.85 (m, 2H), 1.65 (d, J = 11.2 Hz, 2H), 1.49 (s, 3H), 1.26 (d, J = 7.2 Hz, 3H), 1.17 (d, J = 6.8 Hz, 3H), |
| | 0.83 (s, 2H). MS (ESI) |
| | m/z 1812.7 [M + H]⁺ |
| 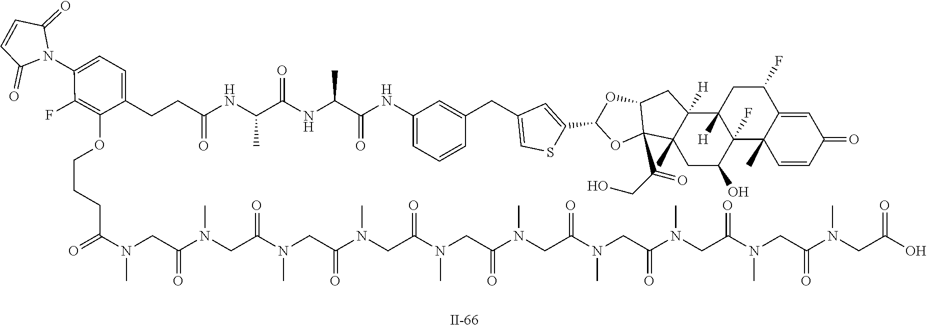 | ¹H NMR (400 MHz, DMSO-d₆) δ = 9.84 (s, 1H), 8.12-8.08 (m, 2H), 7.55-7.39 (m, 2H), 7.28- 7.26 (m, 1H), 7.25 (s, 2H), 7.24-7.16 (m, 3H), 7.14-7.07 (m, 1H), 7.05- 7.01 (m, 2H), 6.88 (d, J = 8.4 Hz, 1H), 6.30 (dd, J = 2.0, 10.0 Hz, 1H), 6.13 (s, 1H), 5.80 (s, 1H), 5.60-5.51 (m, 2H), 4.89 (s, 1H), 4.49 (d, J = 19.6 Hz, 1H), 4.34-3.88 (m, 24H), 3.81 (s, 2H), 2.95-2.75 (m, 34H), 2.68-2.65 (m, 1H), 2.35- 2.31 (m, 3H), 2.06-1.99 (m, 1H), 1.96-1.88 (m, 2H), 1.69-1.60 (m, 3H), 1.49 (s, 3H), 1.30-1.22 |
| | (m, 4H), 1.17 (d, J = 7.2 |
| | Hz, 3H), 0.84 (s, 3H). |
| | MS (ESI) m/z 1811.8 |
| | [M + H]⁺ |
| 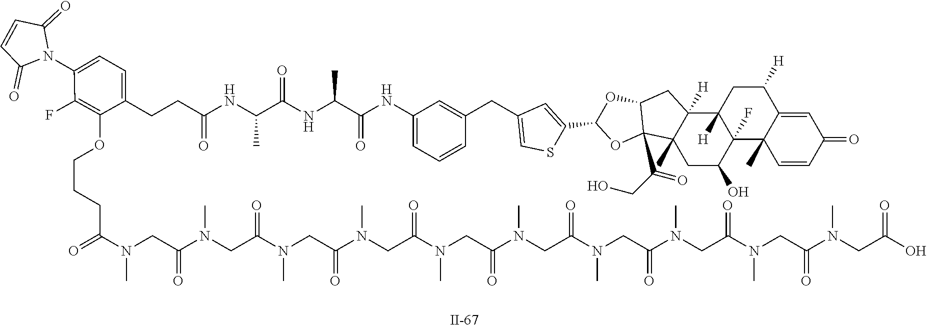 | ¹H NMR (400 MHz, DMSO-d₆) δ = 9.89-9.66 (m, 1H), 8.38-7.98 (m, 2H), 7.58-7.39 (m, 2H), 7.31-7.26 (m, 1H), 7.25- 7.16 (m, 3H), 7.14-6.95 (m, 3H), 6.88 (d, J = 7.6 Hz, 1H), 6.23 (d, J = 9.6 Hz, 1H), 6.08-6.00 (m, 1H), 5.81-5.74 (m, 1H), 5.42 (s, 1H), 4.88 (s, 1H), 4.48 (d, J = 19.2 Hz, 1H), 4.36-3.90 (m, 24H), 3.81 (s, 2H), 3.03- 2.70 (m, 34H), 2.60-2.52 (m, 1H), 2.43-2.29 (m, 2H), 2.28-2.07 (m, 3H), 2.06-1.98 (m, 1H), 1.97- 1.88 (m, 2H), 1.87-1.70 (m, 2H), 1.68-1.57 (m, |
| | 3H), 1.49 (s, 3H), 1.43- |
| | 1.34 (m, 1H), 1.31-1.21 |
| | (m, 3H), 1.18 (d, J = 6.0 |
| | Hz, 3H), 0.84 (s, 3H). |
| | MS (ESI) m/z 1793.8 |
| | [M + H]⁺ |
| 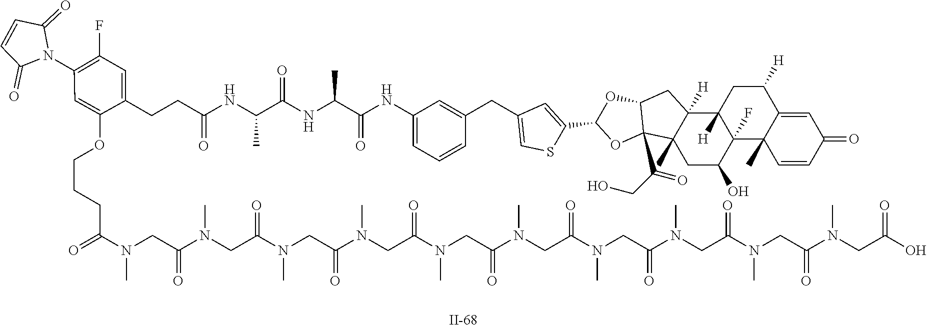 | ¹H NMR (400 MHz, DMSO-d₆) δ = 9.88-9.81 (m, 1H), 8.18-8.04 (m, 2H), 7.55-7.39 (m, 2H), 7.32-7.11 (m, 6H), 7.09- 6.94 (m, 2H), 6.89 (d, J = 7.6 Hz, 1H), 6.27-6.20 (m, 1H), 6.04 (s, 1H), 5.82-5.76 (m, 1H), 5.43 (s, 1H), 4.88 (d, J = 3.2 Hz, 1H), 4.49 (d, J = 19.2 Hz, 1H), 4.42-3.85 (m, 24H), 3.82 (s, 2H), 3.05-2.69 (m, 34H), 2.40-2.35 (m, 1H), 2.08- 1.78 (m, 5H), 1.68-1.58 (m, 3H), 1.49 (s, 3H), 1.27 (d, J = 6.8 Hz, 3H), 1.18 (d, J = 6.8 Hz, 3H), 0.84 (s, 3H). MS (ESI) m/z 1793.7 [M + H]⁺ |
| 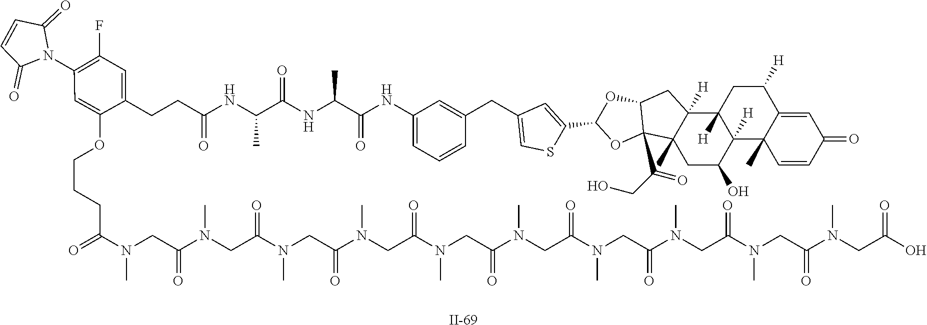 | ¹H NMR (400 MHz, DMSO-d₆) δ = 9.84 (d, J = 2.4 Hz, 1H), 8.14-8.04 (m, 2H), 7.44 (s, 1H), 7.31 (d, J = 9.6 Hz, 1H), 7.23 (s, 2H), 7.22-7.11 (m, 3H), 7.04-6.96 (m, 2H), 6.88 (d, J = 6.8 Hz, 1H), 6.17 (d, J = 9.6 Hz, 1H), 5.95 (s, 1H), 5.81- 5.75 (m, 1H), 4.89-4.78 (m, 2H), 4.52-4.44 (m, 1H), 4.41-3.85 (m, 26H), 3.81 (s, 2H), 3.05-2.69 (m, 34H), 2.25-1.95 (m, 4H), 1.77-1.58 (m, 5H), 1.39 (s, 3H), 1.27 (d, J = 6.4 Hz, 3H), 1.17 (d, J = 7.2 Hz, 3H), 1.01-0.86 (m, 2H), 0.84 (s, 2H). |
| | MS (ESI) m/z 1797.7 |
| | [M + Na]⁺ |
| 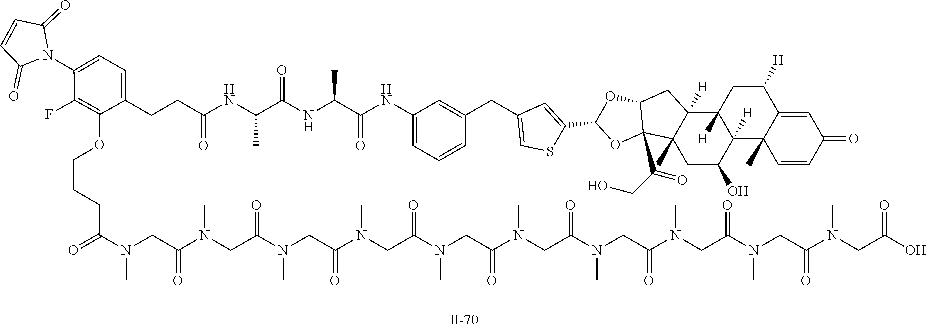 | ¹H NMR (400 MHz, DMSO-d₆) δ = 9.86-9.69 (m, 1H), 8.14-8.07 (m, 2H), 7.55-7.46 (m, 1H), 7.44-7.31 (m, 2H), 7.25 (d, J = 2.0 Hz, 2H), 7.22- 1.15 (m, 2H), 7.10 (d, J = 3.6 Hz, 1H), 7.05-6.99 (m, 2H), 6.90-6.86 (m, 1H), 6.19-6.14 (m, 1H), 5.95 (s, 1H), 5.79-5.74 (m, 1H), 4.87 (d, J = 4.8 Hz, 1H), 4.80 (d, J = 4.4 Hz, 1H), 4.48 (d, J = 19.2 Hz, 1H), 4.43-3.83 (m, 24H), 3.81 (s, 2H), 2.97-2.66 (m, 34H), 2.50-2.33 (m, 2H), 2.17- 1.99 (m, 3H), 1.96-1.87 (m, 2H), 1.73-1.62 (m, 5H), 1.39 (s, 3H), 1.27 |
| | (d, J = 6.4 Hz, 3H), 1.18 |
| | (d, J = 6.4 Hz, 3H), 1.05- |
| | 0.85 (m, 2 H), 0.84 (s, |
| | 2H). MS (ESI) m/z |
| | 1775.7 [M + H]⁺ |

### Biological Example 1. Glucocorticoid Receptor Agonist Activity

Certain GR agonist compounds of the present disclosure were tested in a GR reporter assay. Briefly, CD40 expressing GR-GAL4 Reporter (Luc)-HEK293 or parental GR-GAL4 reporter (Luc)-HEK293 cells were harvested from culture and seeded wells at a density of ˜30,000 cells per well in 45 μl of assay medium. Cells were incubated at 37° C. in a CO₂ incubator overnight. Duplicate five-fold serial dilutions (generally, testing 8 concentrations ranging from 0.005 ng/mL to 1,000 ng/ml) of the tested compound in assay medium were added (5 μl) to stimulated wells and assay medium was added to unstimulated control wells or to cell-free control wells (the latter for determining background luminescence). The plates were incubated at 37° C. in a CO₂ incubator for 24 hours. The Steady-Glo Luciferase Assay System (Promega), following manufacturer's instructions, was used to detect luminescence, which was measured using a luminometer.

The average background luminescence (cell-free control wells) was substracted from the luminescence reading of all wells. The fold induction of GAL4 luciferase reporter expression=background-subtracted luminescence of dexamethasone-stimulated well/average background-subtracted luminescence of unstimulated control wells. The EC₅₀ value for each conjugate was determined in both CD40 expressing and parental GR-GAL4 Reporter (Luc)-HEK293 cells and the ratio was used to as a measure of linker stability and non-specific uptake. The results are summarized in Table 19 below.

TABLE 18
Glucocorticoid Receptor Activity.

| Compound | GR reporter agonist (nM) |
|---|---|
| dexamethasone | 15.6 |
| budesonide | 5.5 |
| 1 | 22.2 |
| 2 | 17.0 |
| 3 | 25.1 |
| 4 | 21.8 |
| 5 | 49.1 |
| 6 | 25.2 |
| 7 | 47.0 |
| 8 | 1.1 |
| 9 | 0.68 |
| 10 | 0.82 |
| 11 | 0.58 |
| 12 | 1.4 |
| 13 | 1.0 |
| 14 | 0.68 |
| 15 | 2.9 |
| 16 | 39.1 |
| 17 | 2.8 |
| 18 | 1.2 |
| 19 | 23.8 |
| 20 | 2.6 |
| 21 | 4.58 |
| 22 | 2.2 |
| 24 | 53.8 |
| 25 | 0.48 |
| 26 | 4.1 |
| 27 | 0.46 |
| 29 | 3.7 |
| 30a | 0.44 |
| 31 | 0.52 |
| 32 | 0.79 |
| 33 | 9.1 |
| 34 | 23.4 |
| 35 | 15.7 |
| 36 | 6.4 |
| 37 | 2.5 |
| 38 | 3.0 |
| 39 | 7.8 |
| 40 | 14.3 |
| 41 | 3.1 |
| 42 | 9.0 |
| 43 | 3.7 |
| 44 | 9.9 |
| 45 | 9.2 |
| 46 | 0.58 |
| 47 | 24 |
| 48 | 0.58 |
| 49 | 27 |
| 50 | 23 |
| 51 | 1.2 |
| 52a | 0.57 |
| 53a | 0.89 |
| 54 | 3.2 |
| 55 | 2.8 |
| 56 | 39.6 |
| 58 | 32.6 |
| 59 | 20.6 |
| 60 | 7.8 |
| 61a | 0.9 |
| 62a | 1.4 |
| 64 | 10.7 |
| 67 | 172 |
| 84 | 2.8 |
| 85 | 4.9 |
| 86a | 0.77 |

### Biological Example 2. CTLA4I2-GR Conjugates with Different Payloads Show CD86 Ligand Dependent Increases in GR Agonism in a Reporter Assay

A HEK293 GR-GAL4 (LUC) reporter cell line (Glucocorticoid Receptor Pathway GR-GAL4 Reporter (Luc)-HEK293 cell line (BPS Bioscience, cat #60655) and transfectants of the reporter line stably expressing the CTLA4Ig ligand human CD86 were plated in growth media in 96 well plates and treated with a dilution series (500 nM-.0064 nM) of the CTLA4Ig (abatacept; SEQ ID NO:101) conjugates (GR agonist dexamethasone (“Dex”) or budesonide (“Bud”) conjugated to abatacept with Linker 1) and a control non-CD86 binding conjugate (Digoxin conjugated to GR agonist dexamethasone with Linker 2) for 24 hours. To determine the level of GR agonism found under the different assay conditions Firefly Luciferase was measured as per manufacturer's instructions with the Steady-Glo Luciferase Assay System (Promega, cat #E2520 or E2550). The data was analyzed and EC₅₀ values determined using Graphpad Prism Software v9.1 (Graphpad Inc.) As seen in Table 20 below, each CTLA4Ig conjugate displayed greater potency on the CD86-expressing HEK293 GR-GAL4 reporter line than the non-CD86-expressing parental line while the non-CD86 binding conjugate displayed the same activity on both cell lines.

TABLE 20
CTLA4Ig conjugates

| | EC₅₀ (nM) | |
|---|---|---|
| | CD86 Reporter Line | Parental Reporter Line |
| CTLA4Ig-Linker 1-Bud | 0.09 | 15.23 |
| CTLA4Ig-Linker 1-Dex | 30.61 | 148.9 |
| Control-Linker 2-Dex | 147.5 | 138.3 |
| (Digoxin) | | |

### Biological Example 3. An Anti-BAFF-GR Conjugate with a GR Agonist, Anti-BAFF-Linker 1-Budenoside Shows Increased BAFF Ligand Directed GR Agonism in a Reporter Cell Assay

A glucocorticoid receptor luciferase reporter cell line stably expressing membrane human BAFF (memBAFF) was generated by transfection of the commercial glucocorticoid receptor cell line (Glucocorticoid Receptor Pathway GR-GAL4 Reporter (Luc)-HEK293 cell line (BPS Bioscience, cat #60655) with an expression vector containing the human BAFF coding region mutated at positions K132A and R122A. For conjugate assay, cells from the BAFF cell line and the parental cell line were plated in complete DMEM growth media in 96 well plates and treated with a dilution series (500 nM-0.0064 nM) of the conjugate (belimumab (heavy chain=SEQ ID NO:9; light chain=SEQ ID NO: 10) conjugated to GR agonist budenoside (“Bud”) with Linker 1) for 24 hours. To determine the level of GR agonism at the end of the incubation luciferase levels were determined with the Steady-Glo Luciferase Assay System (Promega, cat #E2520 or E2550). The results were analyzed with Graphpad Prism software v9.1 (Graphpad Inc) to determine EC50. As seen in Table below, the Anti-BAFF conjugate displayed greater potency on the BIFF-expressing HEK293 GR-GAL4 reporter line than the non-expressing parental line. However, the non-BAFF targeting Anti-HER2 conjugate (Anti-HER2null-Compound 11-83) displayed the same lower potency on non-BAFF and BAFF-expressing lines than seen with the Anti-BAFF conjugates on the BAFF-expressing cell line.

TABLE 21
Anti-BAFF Conjugate

| | | EC₅₀ (nM) | |
|---|---|---|---|
| | | memBAFF reporter | Parental reporter |
| Conjugate | DAR | cell line | cell line |
| Anti-BAFF-Linker 1-Bud | 4.14 | 0.86 | 34.62 |
| (belimumab) | | | |
| Anti-HER2-Linker 1-Bud | 4.51 | 25.94 | 21.81 |
| (SBT050 G1) | | | |

### Biological Example 4. Anti-BAFF-GR Conjugates with the Same Linker but Different GR Agonists Show Ligand-Dependent Increased Potency of GR Agonism in a Cell Reporter Assay

The memBAFF and parental GR-GAL4 luciferase cell reporter cell lines were plated in 96 well plates in complete DMEM media. A dilution series of Anti-BAFF conjugates (belimumab conjugated to GR agonist dexamethasone (“Dex”) or budesonide (“Bud”) with Linker 1) and control conjugate (500 nM-0.0064 nM) were added to the wells followed by 24 hour incubation. To determine the level of GR agonism found for the different treatments, at the end of the incubation firefly luciferase was measured as per manufacturer's instructions with the Steady-Glo Luciferase Assay System (Promega, cat #E2520 or E2550) and then analyzed with Graphpad Prism software (Graphpad Inc v9.1) to determine EC50. As seen in Table 22, the Anti-BAFF-GR conjugates displayed greater potency on the BAFF-expressing HEK293 GR-GAL4 reporter line than the non-expressing parental line. However, the non-BAFF targeting conjugate Anti-HER2FcG1 null displayed a lower potency on the BAFF-expressing line similar to potency on the parental line. The unconjugated Anti-BAFF monoclonal antibody showed no GR agonism on either line over the complete dilution series.

TABLE 22
Anti-BAFF Conjugates with Same Linker
but Different GR Agonist Compounds

| | EC₅₀ (nM) | | |
|---|---|---|---|
| | Mem(BAFF) | Parental | Non-BAFF/ |
| Test Article | line | line | MemBAFF |
| Anti-BAFF-Linker 1-Dex | 3.1 | 139.4* | 45 |
| (belimumab) | | | |
| Anti-BAFF-Linker 1-Bud | 1.35 | 39.59* | 29 |
| (belimumab) | | | |
| Anti-HER2G1null-Linker 1-Bud | 27.98* | 19.2* | 1 |
| BAFF mAb | >500 nM | >500 nM | NA |
| (belimumab) | | | |

*Confidence in linear regression determined EC₅₀ low due to curve fit

### Biological Example 5. LPAM1 Ligand Supports Increased Delivery of Cellular GR Agonism with an Anti-LPAM1 Conjugate, Anti-LPAM-1-Linker 1-Bud, in a Reporter Cell Assay

Stable expression of human LPAM1 was introduced into the GR-GAL4 LUC reporter cell line by transfection of both αv and β7 integrin coding regions in the bi-cistronic expression vector (pIRES-puro3 Takara Bio cat. 631639). Dilution series of an anti-LPAM1 conjugate (vedolizumab conjugated to GR agonist budenoside with Linker 1) and control conjugate (500 nM-0.0064 nM) were added to the wells followed by 24 hour incubation. To determine the level of GR agonism found for the different treatments, at the end of the incubation firefly luciferase was measured as per manufacturer's instructions with the Steady-Glo Luciferase Assay System (Promega, cat #E2520 or E2550). Shown in Table 23 is the ratio of the fold luciferase induction for the anti-LPAM1 conjugate/fold luciferase induction by Anti-HER2 conjugate was high at low concentrations showing the effect of LPAM1-mediated delivery.

TABLE 23
Fold luciferase induction for Anti-LPAMI Conjugate

| | Fold induction | Fold induction | Fold Anti- |
|---|---|---|---|
| | Anti-LPAM1 | Anti-HER2 | LPAM1/Fold |
| Conc (nM) | conjugate | conjugate | Anti-HER2 |
| 0.0064 | 1 | 1 | 1 |
| 0.032 | 1 | 1 | 1 |
| 0.16 | 20 | 1 | 20 |
| 0.8 | 121 | 2 | 60 |
| 4 | 147 | 32 | 5 |
| 20 | 177 | 141 | 1 |
| 100 | 240 | 264 | 1 |
| 500 | 361 | 389 | 1 |

### Biological Example 6. CTLA4I2 and Anti-BAFF conjugates Suppress Dendritic Cell Production of TLR Induced Proinflammatory Cytokines

Mouse dendritic cells (BMDCs) were derived from bone marrow by harvest from femurs and tibia of Balb/c mice and cultured for 6 days under standard culture conditions for dendritic cell production that consisted of supplemented DMEM media and the addition of 40 ng/ml GM-CSF and 20 ng/ml IL-4 on days 0, 3, and 6. BMDCs were then harvested and plated into wells of 96 well plates for continued incubation in media. Dilution series of an Anti-BAFF-Linker 1-Bud (belimumab) conjugate, CTLA4Ig-Linker 2-Bud (abatacept), as well as an unconjugated mAb and CTLA4Ig controls were added to separate wells on both day 6 and day 7 to final concentrations from 150 nM to 0.01 nM. On day 8, 2.5 nM of a TLR7 agonist (2-amino-N-(2-((1-(4-amino-2-(ethoxymethyl)-1H-imidazo[4,5-c]quinolin-1-yl)-2-methylpropan-2-yl)oxy)ethyl)-2-methylpropanamide, Compound 1.2 in Example 1 of WO 2020/056192) was added to wells followed by 6 hours further incubation under growth conditions. Supernatants were then removed and levels of TNFα, IL-6, and IL12p70 were determined by MSD multiplex ELISA and plate reader per manufacturer's instructions (Meso Scale Diagnostics LLC). Only the Anti-BAFF conjugate and CTLA4Ig conjugate treated dendritic cells showed a dose dependent decrease of all 3 proinflammatory cytokines as seen in Table 24, Table 25, and Table 26 as a compared to media and unconjugated proteins (100%).

TABLE 24
TNFα Production Following Conjugate Treatment

| TNFα | Conjugate concentration | | |
|---|---|---|---|
| Conjugate | 0.24 nM | 1.2 nM | 150 nM |
| Anti-BAFF-Linker 1-Bud | 75% | 66% | 20% |
| CTLA4Ig-Linker 2-Bud | 67% | 50% | 17% |

TABLE 25
IL6 Production Following Conjugate Treatment

| IL6 | Conjugate concentration | | |
|---|---|---|---|
| Conjugate | 0.24 nM | 1.2 nM | 150 nM |
| Anti-BAFF-Linker 1-Bud | 73% | 61% | 20% |
| CTLA4Ig-Linker 2-Bud | 78% | 54% | 20% |

TABLE 26
IL12p70 Production Following Conjugate Treatment

| IL12p70 | Conjugate concentration | | |
|---|---|---|---|
| Conjugate | 0.24 nM | 1.2 nM | 150 nM |
| Anti-BAFF-Linker 1-Bud | 71% | 79% | 18% |
| CTLA4Ig-Linker 2-Bud | 60% | 46% | 17% |

### Biological Example 7. An Anti-BAFF Conjugate can Safely Suppress Antigen-Driven Inflammation In Vivo

The ability of Anti-BAFF-Linker 1-Bud (belimumab) conjugate to suppress an in vivo antigen-driven inflammatory response without toxic effects seen with systemic dexamethasone was tested in mice as follows. Female Balb/c mice aged 6-7 weeks (Envigo Labs) were used. On day 0 ear thickness was measured in all mice using microcalipers. On day 0 mice were then sensitized to the antigen fluorescsein isothiocyanate (FITC) on a shaved flank by application of 50 microliters of 1% FITC in a formulation of 50% acetone:50% dibutyl phthalate. On day 6 mice were sorted into treatment groups and treated by ip injection with 20 mpk of Anti-BAFF-Linker 1-Bud, 20 mpk unconjugated anti-BAFF antibody, 10 mpk dexamethasone, or left untreated just prior to rechallenge with 25 μl of 1% FITC on one ear. After 24 hours ear thickness was determined on the treated ear. Mice were then further treated by ip injection with further 20 mpk doses of Anti-BAFF-Linker 1-Bud, unconjugated Anti-BAFF (day 7, day 8), or 10 mpk dexamethasone (days 7-9), or left untreated. Animals were bled for serum and sacrificed on day 9. The serum levels of a marker for healthy bone activity were assessed in serum samples by pro-collagen-1 N-terminal propeptide (P1NP) ELISA using a kit (NovusBio, NBP2-76466) per manufacturer's recommendations. Ear thickness and ELISA results were analyzed for differences in means with GraphPad Prism software by one-way ANOVA with Dunnett's multiple comparisons. Mean ear thickness for both the conjugate and dexamethasone treated groups was significantly different from untreated and unconjugated antibody treated groups at a p<0.01 (Table 27). In addition, comparison of mean values for serum P1NP indicates the conjugate was safer and spared bone metabolism more than dexamethasone (p<0.01) and was not significantly different from the mean for the unconjugated antibody group (Table 28).

TABLE 27
Ear Swelling

| Test Group | Mean Swelling (mm) |
|---|---|
| Untreated | 0.11 |
| Unconjugated belimumab | 0.11 |
| Dexamethasone | 0.07 |
| Anti-BAFF-Linker 1-Bud | 0.02 |

TABLE 28
PN1P serum Levels

| Test Group | Mean P1NP (pg/mL) |
|---|---|
| Dexamethasone | 3977 |
| Unconjugated belimumab | 14029 |
| Anti-BAFF-Linker 1-Bud | 19552 |

### Biological Example 8. A Single Dose of a CTLA4Ig Conjugate Potently Lowers a TLR Response in Dendritic Cells

Mouse dendritic cells (BMDCs) were derived from bone marrow by harvest from femurs and tibia of Balb/c mice and cultured for 6 days under standard culture conditions for dendritic cell production that consisted of supplemented DMEM media and the addition of 40 ng/ml GM-CSF and 20 ng/ml IL-4 on days 0, 3, and 6. BMDCs were then harvested and plated into wells of 96 well plates for continued incubation in media. Dilution series (150 nM to 0.01M) of either an anti-BAFF-Linker 1-Bud (belimumab) conjugate, CTLA4Ig-Linker 2-Bud (abatacept), as well as an unconjugated mAb and CTLA4Ig controls were added followed by a 24 hour incubation, Then 2.5 nM of a TLR7 agonist (2-amino-N-(2-((1-(4-amino-2-(ethoxymethyl)-1H-imidazo[4,5-c]quinolin-1-yl)-2-methylpropan-2-yl)oxy)ethyl)-2-methylpropanamide, Compound 1.2 in Example 1 of WO 2020/056192) was added to wells followed by 6 hours further incubation under growth conditions. Supernatants were removed and levels of TNFα and IL6 were determined by MSD multiplex ELISA and plate reader per manufacturer's instructions (Meso Scale Diagnostics LLC). Both the Anti-BAFF conjugate and CTLA4Ig conjugate treated dendritic cells showed a dose dependent decrease of the proinflammatory cytokines with the CTLA4Ig conjugate showing the highest activity as seen in Table 29 and Table 30. For Table 29, IL6 cytokine levels are shown as a % compared to media and unconjugated proteins (100%).

TABLE 29
IL6 Production Following Conjugate Treatment

| IL6 | Conjugate concentration | | | | |
|---|---|---|---|---|---|
| Conjugate | 0.24 nM | 1.2 nM | 6 nM | 30 nM | 150 nM |
| αBAFF-Linker 1-Bud | 85% | 108% | 96% | 71% | 34% |
| CTLA4Ig-Linker 2-Bud | 55% | 51% | 43% | 35% | 24% |

TABLE 30
TNFα Production Following Conjugate Treatment

| | |
|---|---|
| TNFα | Conjugate concentration |

| Conjugate | 0.4 nM | 1.2 nM | 150 nM |
|---|---|---|---|
| αBAFF-Linker 1-Bud | 75% | 66% | 20% |
| CTLA4Ig-Linker 2-Bud | 67% | 50% | 17% |

### Biological Example 9. In an In Vitro Allo Mixed Lymphocyte Assay Dendritic Cells Treated with Conjugates can Lower T Cell and Dendritic Cell Derived Immunostimulatory Cytokines

Human monocyte derived dendritic cells were derived in vitro from healthy human donor CD 14⁺ monocytes, isolated from blood by standard cell marker negative selection with antibody coated beads, by culture of the monocytes under a standard dendritic cell generation protocol with added GM-CSF and IL4 for 6 days. On day 7, 4×10⁵ dendritic cells were added to wells along with 8×10⁵ CD3⁺ T cells derived from a different allogeneic healthy donor using a pan T cell isolation kit from STEMcell (Cat #17951). Conjugates, protein, and small molecule controls were added at 100 nM immediately after. After 48 hrs of co-culture supernatants were collected from wells and cytokine levels for T cell derived cytokines (IL2, IFNγ) and dendritic cell derived cytokines (IL6, IL 12p40) were measured by MSD multiplex ELISA and plate reader per manufacturer's instructions (Meso Scale Diagnostics LLC). As can be seen in Table 31, small molecule GR agonists dexamethasone (“Dex”) and budesonide (“Bud”), respectively, as well as CTLA4Ig conjugates of these agonists potently depressed immunostimulatory cytokine production. As expected unconjugated CTLA4Ig also had some activity in the assay lowering T cell cytokines. The Anti-BAFF conjugates also showed immunosuppressive activity.

TABLE 31
Cytokine Production Following Conjugate Treatment

| | % Media | | | |
|---|---|---|---|---|
| | IL12p40* | IL6* | IL2* | IFNγ* |
| Media | 100 | 100 | 100 | 100 |
| Dex | 1 | Below | 21 | 2 |
| | | LLOQ** | | |
| Bud | 1 | Below | 14 | 1 |
| | | LLOQ** | | |
| CTLA4Ig-Linker 2-Dex | 11 | 6 | 9 | >1 |
| CTLA4Ig-Linker 2-Bud | 1 | Below | 4 | 3 |
| | | LLOQ** | | |
| CTLA4Ig (abatacept) | 85 | 33 | 14 | 28 |
| Anti-BAFF-Linker 1-Dex | 95 | 66 | 67 | 75 |
| Anti-BAFF-Linker 1-Bud | 60 | 14 | 52 | 52 |
| Anti-BAFF | 139 | 121 | 72 | 140 |
| (belimumab) | | | | |

*Mean of duplicates
**LLOQ lower limit of quantitation in the MSD assay

### Biological Example 10. Additional Conjugates

Additional conjugates as described below were prepared having the characteristics as summarized in Table 32 below.

TABLE 32
Conjugates

| | | | | | | | Max |
|---|---|---|---|---|---|---|---|
| | | | | | | | Fold |
| | GR | | | | Max | | of |
| | Ago- | | | | Fold | EC₅₀ | In- |
| | nist- | | | EC₅₀ | of In- | (nM) | duction |
| | Pay- | | | (nM) | duction | Paren- | Paren- |
| Conj. | load | Binding | | Target | Target | tal | tal |
| # | # | Protein | target | Cells | Cells | Cells | Cells |
| III-1 | II-29 | EPR18005-4- | CD40 | 3.24 | 447.5 | 200 | 132.9 |
| | | mIgG2a null | | | | | |
| | | P329G | | | | | |
| III-2 | II-86 | EPR18005-4- | CD40 | 1.39 | 605.5 | 91.48 | 322.2 |
| | | mIgG2a null | | | | | |
| | | P329G | | | | | |
| III-3 | II-19 | EPR18005-4- | CD40 | 1.24 | 528.3 | 128.3 | 212.5 |
| | | mIgG2a null | | | | | |
| | | P329G | | | | | |
| III-4 | II-8 | EPR18005-4- | CD40 | 2.24 | 501.7 | 177.9 | 175.2 |
| | | mIgG2a null | | | | | |
| | | P329G | | | | | |
| III-5 | II-15 | EPR18005-4- | CD40 | 1.69 | 513.9 | 158.6 | 180.5 |
| | | mIgG2a null | | | | | |
| | | P329G | | | | | |
| III-6 | II-18 | EPR18005-4- | CD40 | 1.01 | 554.2 | 110.9 | 260.9 |
| | | mIgG2a null | | | | | |
| | | P329G | | | | | |
| III-7 | II-14 | EPR18005-4- | CD40 | 0.71 | 614.3 | 48.94 | 501.2 |
| | | mIgG2a null | | | | | |
| | | P329G | | | | | |
| III-8 | II-14 | 65924- | CD40 | 0.72 | 685.5 | 58.77 | 502.1 |
| | | mIgG2a | | | | | |
| | | null P329G | | | | | |
| III-9 | II-19 | bleselumab- | CD40 | 1.11 | 565.9 | 150.5 | 239.6 |
| | | hIgG1 null | | | | | |
| | | P329G | | | | | |
| III-10 | II-18 | bleselumab- | CD40 | 0.79 | 561.4 | 96.03 | 300.3 |
| | | hIgG1 null | | | | | |
| | | P329G | | | | | |
| III-11 | II-19 | bleselumab- | CD40 | 1.15 | 571.3 | 177 | 262.7 |
| | | hIgG1 null | | | | | |
| | | P329G | | | | | |
| III-12 | II-13 | bleselumab- | CD40 | 1.02 | 580.9 | 232.9 | 254.4 |
| | | hIgG1 null | | | | | |
| | | P329G | | | | | |
| III-13 | II-8 | bleselumab- | CD40 | 1.40 | 315.2 | 180.3 | 205.5 |
| | | hIgG1 null | | | | | |
| | | P329G | | | | | |
| III-14 | II-16 | bleselumab- | CD40 | 0.78 | 389.0 | 44.53 | 380.0 |
| | | hIgG1 null | | | | | |
| | | P329G | | | | | |
| III-15 | II-15 | bleselumab- | CD40 | 0.87 | 316.8 | 131 | 248.2 |
| | | hIgG1 null | | | | | |
| | | P329G | | | | | |
| III-16 | II-18 | bleselumab- | CD40 | 0.84 | 348.1 | 100.2 | 326.5 |
| | | hIgG1 null | | | | | |
| | | P329G | | | | | |
| III-17 | II-19 | bleselumab- | CD40 | 0.82 | 314.5 | 126.4 | 294.2 |
| | | hIgG1 null | | | | | |
| | | P329G | | | | | |
| III-18 | II-71 | bleselumab- | CD40 | 0.56 | 350.5 | 73.99 | 474.7 |
| | | hIgG1 null | | | | | |
| | | P329G | | | | | |
| III-19 | II-72 | bleselumab- | CD40 | 0.78 | 291.3 | 4.84 | 545.8 |
| | | hIgG1 null | | | | | |
| | | P329G | | | | | |
| III-20 | II-12 | bleselumab- | CD40 | 0.85 | 269.2 | 23.58 | 497.1 |
| | | hIgG1 null | | | | | |
| | | P329G | | | | | |
| III-21 | II-11 | bleselumab- | CD40 | 0.56 | 278.1 | 142.2 | 342.9 |
| | | hIgG1 null | | | | | |
| | | P329G | | | | | |
| III-22 | II-73 | bleselumab- | CD40 | 1.08 | 319.2 | 110.9 | 307.3 |
| | | hIgG1 null | | | | | |
| | | P329G | | | | | |
| III-23 | II-74 | bleselumab- | CD40 | 0.63 | 338.5 | 72.24 | 392.6 |
| | | hIgG1 null | | | | | |
| | | P329G | | | | | |
| III-24 | II-14 | bleselumab- | CD40 | 0.28 | 326.6 | 97.53 | 377.5 |
| | | hIgG1 null | | | | | |
| | | P329G | | | | | |
| III-25 | II-75 | bleselumab- | CD40 | 1.22 | 356.8 | 11 | 486.3 |
| | | hIgG1 null | | | | | |
| | | P329G | | | | | |
| III-26 | II-72 | bleselumab- | CD40 | 0.50 | 337.3 | 32.53 | 218.7 |
| | | hIgG1 null | | | | | |
| | | P329G | | | | | |
| III-27 | II-76 | bleselumab- | CD40 | 0.37 | 365.0 | 18.66 | 422.6 |
| | | hIgG1 null | | | | | |
| | | P329G | | | | | |
| III-28 | II-77 | bleselumab- | CD40 | 0.18 | 352.7 | 8.85 | 438.9 |
| | | hIgG1 null | | | | | |
| | | P329G | | | | | |
| III-29 | II-78 | bleselumab- | CD40 | 0.40 | 326.1 | 10.37 | 441.3 |
| | | hIgG1 null | | | | | |
| | | P329G | | | | | |
| III-30 | II-79 | bleselumab- | CD40 | 0.27 | 366.4 | 5.08 | 488.6 |
| | | hIgG1 null | | | | | |
| | | P329G | | | | | |
| III-31 | II-80 | bleselumab- | CD40 | 0.36 | 323.9 | 8.28 | 396.0 |
| | | hIgG1 null | | | | | |
| | | P329G | | | | | |
| III-32 | II-78 | bleselumab- | CD40 | 0.62 | 558.2 | 16.12 | 438.8 |
| | | hIgG1 null | | | | | |
| | | P329G | | | | | |
| III-33 | II-79 | 24.2.1-hIgG1 | CD40 | 0.47 | 489.4 | 6.49 | 546.7 |
| | | null P329G | | | | | |
| III-34 | II-78 | 24.2.1-hIgG1 | CD40 | 1.38 | 454.2 | 47.26 | 411.8 |
| | | null P329G | | | | | |
| III-35 | II-81 | 24.2.1-hIgG1 | CD40 | 0.59 | 456.6 | 11.42 | 471.5 |
| | | null P329G | | | | | |
| III-36 | II-78 | 24.2.1-hIgG1 | CD40 | 0.64 | 552.4 | 19.9 | 409.2 |
| | | null P329G | | | | | |
| III-37 | II-82 | adalimumab | TNFα | 0.16 | 500.7 | 101.7 | 457.7 |
| III-38 | II-83 | adalimumab | TNFα | 0.22 | 451.3 | 36.73 | 413.2 |
| III-39 | II-84 | adalimumab | TNFα | 1.25 | 268.1 | 345.5 | 95.4 |
| III-40 | II-85 | adalimumab | TNFα | 0.52 | 322.0 | 148.9 | 227.8 |
| III-41 | II-16 | adalimumab | TNFα | 0.14 | 411.4 | 110.7 | 326.2 |
| III-42 | II-14 | adalimumab | TNFα | 0.15 | 414.7 | 98.52 | 362.6 |
| III-43 | II-83 | adalimumab | TNFα | 0.73 | 439.5 | 38.42 | 485.7 |

SEQUENCES

Belimumab
HCDR1 (SEQ ID NO: 1)
NNAIN
Belimumab HCDR2 (SEQ ID NO: 2)
GIIPMFGTAKYSQNFQG
Belimumab HCDR3 (SEQ ID NO: 3)
SRDLLLFPHHALSP
Belimumab LCDR1 (SEQ ID NO: 4)
QGDSLRSYYAS
Belimumab LCDR2 (SEQ ID NO: 5)
GKNNRPS
Belimumab LCDR3 (SEQ ID NO: 6)
SSRDSSGNHWV
Belimumab VH (SEQ ID NO: 7)
QVQLQQSGAEVKKPGSSVRVSCKASGGTFNNNAINWVRQAPGQGLEWMGGIIPMFGT
AKYSQNFQGRVAITADESTGTASMELSSLRSEDTAVYYCARSRDLLLFPHHALSPWGRG
TMVTVSS
Belimumab VL (SEQ ID NO: 8)
SSELTQDPAVSVALGQTVRVTCQGDSLRSYYASWYQQKPGQAPVLVIYGKNNRPSGIPD
RFSGSSSGNTASLTITGAQAEDEADYYCSSRDSSGNHWVFGGGTELTVLG
Belimumab Heavy Chain (SEQ ID NO: 9)
QVQLQQSGAEVKKPGSSVRVSCKASGGTFNNNAINWVRQAPGQGLEWMGGIIPMFGT
AKYSQNFQGRVAITADESTGTASMELSSLRSEDTAVYYCARSRDLLLFPHHALSPWGRG
TMVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
Belimumab Light Chain (SEQ ID NO: 10)
SSELTQDPAVSVALGQTVRVTCQGDSLRSYYASWYQQKPGQAPVLVIYGKNNRPSGIPD
RFSGSSSGNTASLTITGAQAEDEADYYCSSRDSSGNHWVFGGGTELTVLGQPKAAPSVT
LFPPSSEELQANKATLVCLISDFYPGAVTVAWKADSSPVKAGVETTTPSKQSNNKYAAS
SYLSLTPEQWKSHRSYSCQVTHEGSTVEKTVAPTECS
Tabalumab HCDR1 (SEQ ID NO: 11)
GYYWS
Tabalumab HCDR2 (SEQ ID NO: 12)
EINHSGSTNYNPSLKS
Tabalumab HCDR3 (SEQ ID NO: 13)
GYYDILTGYYYYFDY
Tabalumab LCDR1 (SEQ ID NO: 14)
RASQSVSRYLA
Tabalumab LCDR2 (SEQ ID NO: 15)
DASNRAT
Tabalumab LCDR3 (SEQ ID NO: 16)
QQRSNWPRT
Tabalumab VH (SEQ ID NO: 17)
QVQLQQWGAGLLKPSETLSLTCAVYGGSFSGYYWSWIRQPPGKGLEWIGEINHSGSTN
YNPSLKSRVTISVDTSKNQFSLKLSSVTAADTAVYYCARGYYDILTGYYYYFDYWGQG
TLVTVSS
Tabalumab VL (SEQ ID NO: 18)
EIVLTQSPATLSLSPGERATLSCRASQSVSRYLAWYQQKPGQAPRLLIYDASNRATGIPA
RFSGSGSGTDSTLTISSLEPEDFAVYYCQQRSNWPRTFGQGTKVEIK
Tabalumab Heavy Chain (SEQ ID NO: 19)
QVQLQQWGAGLLKPSETLSLTCAVYGGSFSGYYWSWIRQPPGKGLEWIGEINHSGSTN
YNPSLKSRVTISVDTSKNQFSLKLSSVTAADTAVYYCARGYYDILTGYYYYFDYWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
Tabalumab Heavy Chain from tibulizumab (SEQ ID NO: 19)
QVQLQQWGAGLLKPSETLSLTCAVYGGSFSGYYWSWIRQPPGKGLEWIGEINHSGSTN
YNPSLKSRVTISVDTSKNQFSLKLSSVTAADTAVYYCARGYYDILTGYYYYFDYWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSPG
Tabalumab Light Chain (SEQ ID NO: 20)
EIVLTQSPATLSLSPGERATLSCRASQSVSRYLAWYQQKPGQAPRLLIYDASNRATGIPA
RFSGSGSGTDSTLTISSLEPEDFAVYYCQQRSNWPRTFGQGTKVEIKRTVAAPSVFIFPPS
DEQLKSGTASVVCLLNNFYPREAKVQWKVDNALQSGNSQESVTEQDSKDSTYSLSNTL
TLSKADYEKHKVYACEVTHQGLSSPVTKSFNRGEC
rozibafusp alfa HCDR1 (SEQ ID NO: 21)
SYWMS
rozibafusp alfa HCDR2 (SEQ ID NO: 22)
YIKQDGNEKYYVDSVKG
rozibafusp alfa HCDR3 (SEQ ID NO: 23)
EGILWFGDLPTF
rozibafusp alfa LCDR1 (SEQ ID NO: 24)
RASQGISNWLA
rozibafusp alfa LCDR2 (SEQ ID NO: 25)
AASSLQS
rozibafusp alfa LCDR3 (SEQ ID NO: 26)
QQYDSYPRT
rozibafusp alfa anti-ICOSL VH (SEQ ID NO: 27)
EVQLVESGGGLVQPGGSLRLSCAASGFTFSSYWMSWVRQAPGKGLEWVAYIKQDGNE
KYYVDSVKGRFTISRDNAKNSLYLQMNSLRAEDTAVYYCAREGILWFGDLPTFWGQGT
LVTVSS
rozibafusp alfa anti-ICOSL VL (SEQ ID NO: 28)
DIQMTQSPSSLSASVGDRVTITCRASQGISNWLAWYQQKPEKAPKSLIYAASSLQSGVPS
RFSGSGSGTDFTLTISSLQPEDFATYYCQQYDSYPRTFGQGTKVEIK
rozibafusp alfa anti-ICOSL Light Chain (SEQ ID NO: 29)
DIQMTQSPSSLSASVGDRVTITCRASQGISNWLAWYQQKPEKAPKSLIYAASSLQSGVPS
RFSGSGSGTDFTLTISSLQPEDFATYYCQQYDSYPRTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
rozibafusp alfa anti-ICOSL Heavy Chain (SEQ ID NO: 30)
EVQLVESGGGLVQPGGSLRLSCAASGFTFSSYWMSWVRQAPGKGLEWVAYIKQDGNE
KYYVDSVKGRFTISRDNAKNSLYLQMNSLRAEDTAVYYCAREGILWFGDLPTFWGQGT
LVTVSSASTKGPSVFPLAPCSRSTSESTAALGCLVKDYFPEPVTVSWNSGALTSGVHTFP
AVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSDGSFFLYSKL
TVDKSRWQQGNVFSCSVMHEALHNHYTQKSLSLSPGKG
rozibafusp alfa bispecific ICOSL Heavy Chain with 2x BAFF binding protein fusion (SEQ ID
NO: 31)
EVQLVESGGGLVQPGGSLRLSCAASGFTFSSYWMSWVRQAPGKGLEWVAYIKQDGNE
KYYVDSVKGRFTISRDNAKNSLYLQMNSLRAEDTAVYYCAREGILWFGDLPTFWGQGT
LVTVSSASTKGPSVFPLAPCSRSTSESTAALGCLVKDYFPEPVTVSWNSGALTSGVHTFP
AVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSDGSFFLYSKL
TVDKSRWQQGNVFSCSVMHEALHNHYTQKSLSLSPGKGGGGGLPGCKWDLLIKQWVC
DPLGSGSATGGSGSVASSGSGSATHLLPGCKWDLLIKQWVCDPL
rozibafusp alfa Heavy Chain Linker (SEQ ID NO: 32)
GGGG
rozibafusp alfaBAFF binding protein Linker (SEQ ID NO: 33)
GSGSATGGSGSVASSGSGSATHL
rozibafusp alfaBAFF-Binding Protein (SEQ ID NO: 34)
LPGCKWDLLIKQWVCDPL
Blisibimod (fusion protein)(SEQ ID NO: 35)
GCKWDLLIKQWVCDPLGSGSATGGSGSTASSGSGSATHMLPGCKWDLLIKQWVCDPL
GGGGGVDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKSLSLSPGK
Blisibimod BAFF binding protein (SEQ ID NO: 36)
GCKWDLLIKQWVCDPL
Blisibimod BAFF binding protein (SEQ ID NO: 37)
LPGCKWDLLIKQWVCDPL
Blisibimod BAFF binding protein Linker (SEQ ID NO: 38)
GSGSATGGSGSTASSGSGSATHM
Blisibimod
Linker to hIgG1 (SEQ ID NO: 39)
GGGGGV
TACI ectodomain (blocks BAFF and APRIL binding)(SEQ ID NO: 40)
AMRSCPEEQYWDPLLGTCMSCKTICNHQSQRTCAAFCRSLSCRKEQGKFYDHLLRDCIS
CASICGQHPKQCAYFCENKLRS
Atacicept (TACI ectodomain fused to hIgG1 Fc)(SEQ ID NO: 41)
AMRSCPEEQYWDPLLGTCMSCKTICNHQSQRTCAAFCRSLSCRKEQGKFYDHLLRDCIS
CASICGQHPKQCAYFCENKLRSEPKSSDKTHTCPPCPAPEAEGAPSVFLFPPKPKDTLMIS
RTPEVTCVVVDVSHEDPEVKFNWYVDGVEVHNAKTKPREEQYNSTYRVVSVLTVLHQ
DWLNGKEYKCKVSNKALPSSIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVM
HEALHNHYTQKSLSLSPGK
Tibulizumab anti-BAFF tabalumab Heavy Chain fused to anti-IL-17 scFv from ixekizumab
(combine with LC SEQ ID NO: 20)(SEQ ID NO: 42)
QVQLQQWGAGLLKPSETLSLTCAVYGGSFSGYYWSWIRQPPGKGLEWIGEINHSGSTN
YNPSLKSRVTISVDTSKNQFSLKLSSVTAADTAVYYCARGYYDILTGYYYYFDYWGQG
TLVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSPGGGGSGGGGTGGGGSQVQLVQSG
AEVKKPGSSVKVSCKASGYKFTDYHIHWVRQAPGQCLEWMGVINPTYGTTDYNQRFK
GRVTITADESTSTAYMELSSLRSEDTAVYYCARYDYFTGTGVYWGQGTLVTVSSGGGG
SGGGGSGGGGSGGGGSDIVMTQTPLSLSVTPGQPASISCRSSRSLVHSRGETYLHWYLQ
KPGQSPQLLIYKVSNRFIGVPDRFSGSGSGTDFTLKISRVEAEDVGVYYCSQSTHLPFTFG
CGTKLEIK
Ixekizumab anti-IL-17 VH from tibulizumab (SEQ ID NO: 43)
QVQLVQSGAEVKKPGSSVKVSCKASGYKFTDYHIHWVRQAPGQCLEWMGVINPTYGT
TDYNQRFKGRVTITADESTSTAYMELSSLRSEDTAVYYCARYDYFTGTGVYWGQGTLV
TVSS
Ixekizumab anti-IL-17 VL from tibulizumab (SEQ ID NO: 44)
DIVMTQTPLSLSVTPGQPASISCRSSRSLVHSRGETYLHWYLQKPGQSPQLLIYKVSNRFI
GVPDRFSGSGSGTDFTLKISRVEAEDVGVYYCSQSTHLPFTFGCGTKLEIK
Ixekizumab anti-IL-17 scFv (SEQ ID NO: 45)
QVQLVQSGAEVKKPGSSVKVSCKASGYKFTDYHIHWVRQAPGQCLEWMGVINPTYGT
TDYNQRFKGRVTITADESTSTAYMELSSLRSEDTAVYYCARYDYFTGTGVYWGQGTLV
TVSSGGGGSGGGGSGGGGSGGGGSDIVMTQTPLSLSVTPGQPASISCRSSRSLVHSRGET
YLHWYLQKPGQSPQLLIYKVSNRFIGVPDRFSGSGSGTDFTLKISRVEAEDVGVYYCSQS
THLPFTFGCGTKLEIK
Tibulizumab Heavy Chain Linker (SEQ ID NO: 46)
GGGSGGGGTGGGGS
Tibulizumab anti-IL-17 scFv Linker (SEQ ID NO: 47)
GGGGSGGGGSGGGGSGGGGS
BAFF-R Ectodomain (SEQ ID NO: 48)
DVRRGPRSLRGRDAPAPTPCNPAECFDPLVRHCVACGLLRTPRPKPAGASSPAPRTALQP
QESVGAGAGEAA
Briobacept (BAFF-R ectodomain fused to hIgG1 Fc)(SEQ ID NO: 49)
DVRRGPRSLRGRDAPAPTPCNPAECFDPLVRHCVACGLLRTPRPKPAGASSPAPRTALQP
QESVGAGAGEAAVDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSL
SLSPG
Ianalumab VH (SEQ ID NO: 50)
GRIYYRSKWYNSYAVSVKSRITINPDTSKNQFSLQLNSVTPEDTAVYYCARYQWVPKIG
VFDSWGQGTLVTVSS
Ianalumab VL (SEQ ID NO: 51)
DIVLTQSPATLSLSPGERATLSCRASQFILPEYLSWYQQKPGQAPRLLIYGSSSRATGVPA
RFSGSGSGTDFTLTISSLEPEDFAVYYCQQFYSSPLTFGQGTKVEIK
Ianalumab Heavy Chain (SEQ ID NO: 52)
GRIYYRSKWYNSYAVSVKSRITINPDTSKNQFSLQLNSVTPEDTAVYYCARYQWVPKIG
VFDSWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCD
KTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Ianalumab Light Chain (SEQ ID NO: 53)
DIVLTQSPATLSLSPGERATLSCRASQFILPEYLSWYQQKPGQAPRLLIYGSSSRATGVPA
RFSGSGSGTDFTLTISSLEPEDFAVYYCQQFYSSPLTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Vedolizumab VH (SEQ ID NO: 54)
QVQLVQSGAEVKKPGASVKVSCKGSGYTFTSYWMHWVRQAPGQRLEWIGEIDPSESNT
NYNQKFKGRVTLTVDISASTAYMELSSLRSEDTAVYYCARGGYDGWDYAIDYWGQGT
LVTVSS
Vedolizumab VL (SEQ ID NO: 55)
DVVMTQSPLSLPVTPGEPASISCRSSQSLAKSYGNTYLSWYLQKPGQSPQLLIYGISNRFS
GVPDRFSGSGSGTDFTLKISRVEAEDVGVYYCLQGTHQPYTFGQGTKVEIK
Vedolizumab Heavy Chain (SEQ ID NO: 56)
QVQLVQSGAEVKKPGASVKVSCKGSGYTFTSYWMHWVRQAPGQRLEWIGEIDPSESNT
NYNQKFKGRVTLTVDISASTAYMELSSLRSEDTAVYYCARGGYDGWDYAIDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELAGAPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
Vedolizumab Light Chain (SEQ ID NO: 57)
DVVMTQSPLSLPVTPGEPASISCRSSQSLAKSYGNTYLSWYLQKPGQSPQLLIYGISNRFS
GVPDRFSGSGSGTDFTLKISRVEAEDVGVYYCLQGTHQPYTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Etrolizumab VH (SEQ ID NO: 58)
EVQLVESGGGLVQPGGSLRLSCAASGFFITNNYWGWVRQAPGKGLEWVGYISYSGSTS
YNPSLKSRFTISRDTSKNTFYLQMNSLRAEDTAVYYCARTGSSGYFDFWGQGTLVTVSS
Etrolizumab VL (SEQ ID NO: 59)
DIQMTQSPSSLSASVGDRVTITCRASESVDDLLHWYQQKPGKAPKLLIKYASQSISGVPS
RFSGSGSGTDFTLTISSLQPEDFATYYCQQGNSLPNTFGQGTKVEIK
Etrolizumab Heavy Chain (SEQ ID NO: 60)
EVQLVESGGGLVQPGGSLRLSCAASGFFITNNYWGWVRQAPGKGLEWVGYISYSGSTS
YNPSLKSRFTISRDTSKNTFYLQMNSLRAEDTAVYYCARTGSSGYFDFWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPG
Etrolizumab Light Chain (SEQ ID NO: 61)
DIQMTQSPSSLSASVGDRVTITCRASESVDDLLHWYQQKPGKAPKLLIKYASQSISGVPS
RFSGSGSGTDFTLTISSLQPEDFATYYCQQGNSLPNTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGEC
Bleselumab VH (SEQ ID NO: 62)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
S
Bleselumab VL (SEQ ID NO: 63)
ASNLESGVPSRFSGSGSGTDFTLTISSLQPEDFATYYCQQFNSYPTFGQG1TKVEIK
Bleselumab Heavy Chain (SEQ ID NO: 64)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPS
VFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSR
WQEGNVFSCSVMHEALHNHYTQKSLSLSLGK
Bleselumab Light Chain (SEQ ID NO: 65)
AIQLTQSPSSLSASVGDRVTITCRASQGISSLAWYQQKPGKAPKLLIYDASNLESGVPSRF
SGSGSGTDFTLTISSLQPEDFATYYCQQFNSYPTFGQG1TKVEIKRTVAAPSVFIFPPSDEQ
LKSGTASVVCLLNNFYPREAKVQWKVDNALQSGNSQESVTEQDSKDSTYSLSSTLTLSK
ADYEKHKVYACEVTHQGLSSPVTKSFNRGEC
Iscalimab (or lucatumumab) VH (SEQ ID NO: 66)
QVQLVESGGGVVQPGRSLRLSCAASGFTFSSYGMHWVRQAPGKGLEWVAVISYEESNR
YHADSVKGRFTISRDNSKITLYLQMNSLRTEDTAVYYCARDGGIAAPGPDYWGQGTLV
TVSS
Iscalimab (or lucatumumab) VL (SEQ ID NO: 67)
DIVMTQSPLSLTVTPGEPASISCRSSQSLLYSNGYNYLDWYLQKPGQSPQVLISLGSNRAS
GVPDRFSGSGSGTDFTLKISRVEAEDVGVYYCMQARQTPFTFGPGTKVDIR
Iscalimab Heavy Chain (SEQ ID NO: 68)
QVQLVESGGGVVQPGRSLRLSCAASGFTFSSYGMHWVRQAPGKGLEWVAVISYEESNR
YHADSVKGRFTISRDNSKITLYLQMNSLRTEDTAVYYCARDGGIAAPGPDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSCDKTHTCPPCPAPE
LLGGPSVFLFPPKPKDTLMISRTPEVTCVVVDVSHEDPEVKFNWYVDGVEVHNAKTKPR
EEQYASTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
Iscalimab Light Chain (SEQ ID NO: 69)
DIVMTQSPLSLTVTPGEPASISCRSSQSLLYSNGYNYLDWYLQKPGQSPQVLISLGSNRAS
GVPDRFSGSGSGTDFTLKISRVEAEDVGVYYCMQARQTPFTFGPGTKVDIRRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Ravagalimab VH (SEQ ID NO: 70)
EVQLVESGGGLVKPGGSLRLSCAASGFTFSDYGMNWVRQAPGKGLEWIAYISSGRGNI
YYADTVKGRFTISRDNAKNSLYLQMNSLRAEDTAVYYCARSWGYFDVWGQGTTVTVS
S
Ravagalimab VL (SEQ ID NO: 71)
DIVMTQSPDSLAVSLGERATINCKSSQSLLNRGNQKNYLTWFQQKPGQPPKLLIYWAST
RESGVPDRFSGSGSGTDFTLTISSLQAEDVAVYYCONDYTYPLTFGQGTKLEIK
Ravagalimab Heavy Chain (SEQ ID NO: 72)
EVQLVESGGGLVKPGGSLRLSCAASGFTFSDYGMNWVRQAPGKGLEWIAYISSGRGNI
YYADTVKGRFTISRDNAKNSLYLQMNSLRAEDTAVYYCARSWGYFDVWGQGTTVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAG
GPSVFLFPPKPKDQLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLYSKLTV
DKSRWQQGNVFSCSVLHEALHNHYTQKSLSLSPGK
Ravagalimab Light Chain (SEQ ID NO: 73)
DIVMTQSPDSLAVSLGERATINCKSSQSLLNRGNQKNYLTWFQQKPGQPPKLLIYWAST
RESGVPDRFSGSGSGTDFTLTISSLQAEDVAVYYCONDYTYPLTFGQGTKLEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
Dacetuzumab VH (SEQ ID NO: 74)
EVQLVESGGGLVQPGGSLRLSCAASGYSFTGYYIHWVRQAPGKGLEWVARVIPNAGGT
SYNQKFKGRFTLSVDNSKNTAYLQMNSLRAEDTAVYYCAREGIYWWGQGTLVTVSS
Dacetuzumab VL (SEQ ID NO: 75)
DIQMTQSPSSLSASVGDRVTITCRSSQSLVHSNGNTFLHWYQQKPGKAPKLLIYTVSNRF
SGVPSRFSGSGSGTDFTLTISSLQPEDFATYFCSQTTHVPWTFGQGTKVEIK
Giloralimab VH (SEQ ID NO: 76)
EVQLQESGPGLVKPSETLSLTCTVSGYSITSNYYWNWIRQPPGKGLEWMGYIRYDGSNN
YNPSLKNRVTISRDTSKNQFSLKLSSVTAADTAVYYCARLDYWGQGTTVTVSS
Giloralimab VL (SEQ ID NO: 77)
DIVMTQTPLSLSVTPGQPASISCRSSQSLENTNGNTFLNWYLQKPGQSPQLLIYRVSNRFS
GVPDRFSGSGSGTDFTLKISRVEAEDVGVYYCLQVTHVPFTFGQGTKLEIK
Mitazalimab VH (SEQ ID NO: 78)
EVQLLESGGGLVQPGGSLRLSCAASGFTFSTYGMHWVRQAPGKGLEWLSYISGGSSYIF
YADSVRGRFTISRDNSENALYLQMNSLRAEDTAVYYCARILRGGSGMDLWGQGTLVTV
SS
Mitazalimab VL (SEQ ID NO: 79)
QSVLTQPPSASGTPGQRVTISCTGSSSNIGAGYNVYWYQQLPGTAPKLLIYGNINRPSGV
PDRFSGSKSGTSASLAISGLRSEDEADYYCAAWDKSISGLVFGGGTKLTVL
Selicrelumab VH (SEQ ID NO: 80)
QVQLVQSGAEVKKPGASVKVSCKASGYTFTGYYMHWVRQAPGQGLEWMGWINPDSG
GTNYAQKFQGRVTMTRDTSISTAYMELNRLRSDDTAVYYCARDQPLGYCTNGVCSYF
DYWGQGTLVTVSS
Selicrelumab VL (SEQ ID NO: 81)
DIQMTQSPSSVSASVGDRVTITCRASQGIYSWLAWYQQKPGKAPNLLIYTASTLQSGVPS
RFSGSGSGTDFTLTISSLQPEDFATYYCQQANIFPLTFGGGTKVEIK
Sotigalimab VH (SEQ ID NO: 82)
QVQLVESGGGVVQPGRSLRLSCAASGFSFSSTYVCWVRQAPGKGLEWIACIYTGDGTN
YSASWAKGRFTISKDSSKNTVYLQMNSLRAEDTAVYFCARPDITYGFAINFWGPGTLVT
VSS
Sotigalimab VL (SEQ ID NO: 83)
DIQMTQSPSSLSASVGDRVTIKCQASQSISSRLAWYQQKPGKPPKLLIYRASTLASGVPSR
FSGSGSGTDFTLTISSLQPEDVATYYCQCTGYGISWPIGGGTKVEIK
Vanalimab VH (SEQ ID NO: 84)
EVQLLESGGGLVQPGGSLRLSCAASGFTFSTYGMHWVRQAPGKGLEWLSYISGGSSYIF
YADSVRGRFTISRDNSENALYLQMNSLRAEDTAVYYCARILRGGSGMDLWGQGTLVTV
SS
Vanalimab VL (SEQ ID NO: 85)
QSVLTQPPSASGTPGQRVTISCTGSSSNIGAGYNVYWYQQLPGTAPKLLIYGNINRPSGV
PDRFSGSKSGTSASLAISGLRSEDEADYYCAAWDKSISGLVFGGGTKLTVL
dapirolizumab pego1 VH (SEQ ID NO: 86)
EVQLVESGGGLVQPGGSLRLSCAVSGFSSTNYHVHWVRQAPGKGLEWMGVIWGDGDT
SYNSVLKSRFTISRDTSKNTVYLQMNSLRAEDTAVYYCARQLTHYYVLAAWGQGTLVT
VSS
dapirolizumab pegol VL (SEQ ID NO: 87)
DIQMTQSPSSLSASVGDRVTITCRASEDLYYNLAWYQRKPGKAPKLLIYDTYRLADGVP
SRFSGSGSGTDYTLTISSLQPEDFASYYCQQYYKFPFTFGQGTKVEIK
dapirolizumab pego1 Heavy Chain (SEQ ID NO: 88)
EVQLVESGGGLVQPGGSLRLSCAVSGFSSTNYHVHWVRQAPGKGLEWMGVIWGDGDT
SYNSVLKSRFTISRDTSKNTVYLQMNSLRAEDTAVYYCARQLTHYYVLAAWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCAA
dapirolizumab pego1 Light Chain (SEQ ID NO: 89)
DIQMTQSPSSLSASVGDRVTITCRASEDLYYNLAWYQRKPGKAPKLLIYDTYRLADGVP
SRFSGSGSGTDYTLTISSLQPEDFASYYCQQYYKFPFTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
letolizumab VH (SEQ ID NO: 90)
EVQLLESGGGLVQPGGSLRLSCAASGFTFNWELMGWARQAPGKGLEWVSGIEGPGDVT
YYADSVKGRFTISRDNSKNTLYLQMNSLRAEDTAVYYCVKVGKDAKSDYRGQGTLVT
VSS
letolizumab (anti-CD40L IgG1 Fc fusion protein)(SEQ ID NO: 91)
EVQLLESGGGLVQPGGSLRLSCAASGFTFNWELMGWARQAPGKGLEWVSGIEGPGDVT
YYADSVKGRFTISRDNSKNTLYLQMNSLRAEDTAVYYCVKVGKDAKSDYRGQGTLVT
VSSASTEPKSSDKTHTSPPSPAPELLGGSSVFLFPPKPKDTLMISRTPEVTCVVVDVSHED
PEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSP
GK
Dazodalibep (anti-CD40L-HSA fusion protein)(SEQ ID NO: 92)
SQIEVKDVTDTTALITWSDDFGEYVWCELTYGIKDVPGDRTTIDLWYHHAHYSIGNLKP
DTEYEVSLICRSGDMSSNPAKETFTTGGGGGGGGGGGGGGGRLDAPSQIEVKDVTDTT
ALITWSDDFGEYVWCELTYGIKDVPGDRTTIDLWYHHAHYSIGNLKPDTEYEVSLICRS
GDMSSNPAKETFTTGGGGGGGGGGDAHKSEVAHRFKDLGEENFKALVLIAFAQYLQQS
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFYA
PELLFFAKRYKAAFTECCQAADKAACLLPKLDELRDEGKASSAKQRLKCASLQKFGER
AFKAWAVARLSQRFPKAEFAEVSKLVTDLTKVHTECCHGDLLECADDRADLAKYICEN
QDSISSKLKECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGSKCCKHPEA
KRMPCAEDYLSVVLNQLCVLHEKTPVSDRVTKCCTESLVNRRPCFSALEVDETYVPKEF
NAETFTFHADICTLSEKERQIKKQTALVELVKHKPKATKEQLKAVMDDFAAFVEKCCK
ADDKETCFAEEGKKLVAASQAALGL
anti-CD40L var1 (SEQ ID NO: 93)
SQIEVKDVTDTTALITWSDDFGEYVWCELTYGIKDVPGDRTTIDLWYHHAHYSIGNLKP
DTEYEVSLICRSGDMSSNPAKETFTT
anti-CD40L var2 (SEQ ID NO: 94)
RLDAPSQIEVKDVTDTTALITWSDDFGEYVWCELTYGIKDVPGDRTTIDLWYHHAHYSI
GNLKPDTEYEVSLICRSGDMSSNPAKETFTT
human serum albumin (SEQ ID NO: 95)
DAHKSEVAHRFKDLGEENFKALVLIAFAQYLQQSPFEDHVKLVNEVTEFAKTCVADES
AENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVS
KLVTDLTKVHTECCHGDLLECADDRADLAKYICENQDSISSKLKECCEKPLLEKSHCIAE
VENDEMPADLPSLAADFVESKDVCKNYAEAKDVFLGMFLYEYARRHPDYSVVLLLRL
AKTYETTLEKCCAAADPHECYAKVFDEFKPLVEEPQNLIKQNCELFEQLGEYKFQNALL
VRYTKKVPQVSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICTLSEKERQIKKQ
TALVELVKHKPKATKEQLKAVMDDFAAFVEKCCKADDKETCFAEEGKKLVAASQAAL
GL
anti-CD40L Gly linker (SEQ ID NO: 96)
GGGGGGGGGGGGGGG
albumin linker (SEQ ID NO: 97)
GGGGGGGGGG
CTLA4 Ectodomain (high affinity variant from belatacept)(SEQ ID NO: 98)
MHVAQPAVVLASSRGIASFVCEYASPGKYTEVRVTVLRQADSQVTEVCAATYMMGNE
LTFLDDSICTGTSSGNQVNLTIQGLRAMDTGLYICKVELMYPPPYYEGIGNGTQIYVIDPE
PCPDSDQ
Belatacept fusion protein (SEQ ID NO: 99)
MHVAQPAVVLASSRGIASFVCEYASPGKYTEVRVTVLRQADSQVTEVCAATYMMGNE
LTFLDDSICTGTSSGNQVNLTIQGLRAMDTGLYICKVELMYPPPYYEGIGNGTQIYVIDPE
PCPDSDQEPKSSDKTHTSPPSPAPELLGGSSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSL
SPGK
CTLA4 Ectodomain from abatacept (SEQ ID NO: 100)
MHVAQPAVVLASSRGIASFVCEYASPGKATEVRVTVLRQADSQVTEVCAATYMMGNE
LTFLDDSICTGTSSGNQVNLTIQGLRAMDTGLYICKVELMYPPPYYLGIGNGTQIYVIDPE
PCPDSDQ
Abatacept CTLA4-hIgG1 Fc fusion protein (SEQ ID NO: 101)
MHVAQPAVVLASSRGIASFVCEYASPGKATEVRVTVLRQADSQVTEVCAATYMMGNE
LTFLDDSICTGTSSGNQVNLTIQGLRAMDTGLYICKVELMYPPPYYLGIGNGTQIYVIDPE
PCPDSDQEPKSSDKTHTSPPSPAPELLGGSSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSL
SPGK
CTLA4 Ectodomain Asp2408 variant from abatacept (SEQ ID NO: 102)
MHVAQPAVVLASSRGIASFVCEYASPGKANEVRVTVLRQADSQVTEVCAMTYMKENE
LTFLDDPICTGTFSGNQVNLTIQGLRAMDTGLYICKVELMYPPPYYLGIGNGTQIYVIDPE
PCPDSDQ
Abatacept variant 1 CTLA4-hIgG1 Fc (CTLA4 Asp2408 variant)(SEQ ID NO: 103)
MHVAQPAVVLASSRGIASFVCEYASPGKANEVRVTVLRQADSQVTEVCAMTYMKENE
LTFLDDPICTGTFSGNQVNLTIQGLRAMDTGLYICKVELMYPPPYYLGIGNGTQIYVIDPE
PCPDSDQEPKSSDKTHTSPPSPAPELLGGSSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSL
SPGK
CTLA4 Ectodomain Asp2409 variant from abatacept (SEQ ID NO: 104)
MHVAQPAVVLASSRGIASFVCEYESPGKANEIRVTVLRQADSQVTEVCAMTYMKGDEL
TFLDDPSCTGTFSGNQVNLTIQGLRAMDTGLYICKVELMYPPPYYEGIGNGTQIYVIDPE
PCPDSDQ
Abatacept variant 2 CTLA4-hIgG1 Fc (CTLA4 Asp2409 variant)(SEQ ID NO: 105)
MHVAQPAVVLASSRGIASFVCEYESPGKANEIRVTVLRQADSQVTEVCAMTYMKGDEL
TFLDDPSCTGTFSGNQVNLTIQGLRAMDTGLYICKVELMYPPPYYEGIGNGTQIYVIDPE
PCPDSDQEPKSSDKTHTSPPSPAPELLGGSSVFLFPPKPKDTLMISRTPEVTCVVVDVSHE
DPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSL
SPGK
Alomfilimab VH (SEQ ID NO: 106)
EVQLVESGGGVVRPGGSLRLSCVASGVTFDDYGMSWVRQAPGKGLEWVSGINWNGG
DTDYSDSVKGRFTISRDNAKNSLYLQMNSLRAEDTALYYCARDFYGSGSYYHVPFDYW
GQGILVTVSS
Alomfilimab VL (SEQ ID NO: 107)
EIVLTQSPGTLSLSPGERATLSCRASQSVSRSYLAWYQQKRGQAPRLLIYGASSRATGIPD
RFSGDGSGTDFTLSISRLEPEDFAVYYCHQYDMSPFTFGPGTKVDIK
Feladilimab VH (SEQ ID NO: 108)
QVQLVQSGAEVKKPGSSVKVSCKASGYTFTDYAMHWVRQAPGQGLEWMGLISIYSDH
TNYNQKFQGRVTITADKSTSTAYMELSSLRSEDTAVYYCGRNNYGNYGWYFDVWGQG
TTVTVSS
Feladilimab VL (SEQ ID NO: 109)
EIVLTQSPATLSLSPGERATLSCSASSSVSYMHWYQQKPGQAPRLLIYDTSKLASGIPARF
SGSGSGTDYTLTISSLEPEDFAVYYCFQGSGYPYTFGQGTKLEIK
Izuralimab (Bispecific mAb / scFv targeting ICOS and PD-1; ICOS binding domain provided)
VH (SEQ ID NO: 110)
QVQLVQSGAEVKKPGASVKVSCKASGYTFTGYYMHWVRQAPGQGLEWMGWINPHSG
GTNYAQKFQGRVTMTRDTSISTAYMELSRLRSDDTAVYYCARTYYYDSSGYYHDAFDI
WGQGTMVTVSS
Feladilimab VL (SEQ ID NO: 111)
DIQMTQSPSSVSASVGDRVTITCRASQGISRLLAWYQQKPGKAPKLLIYVASSLQSGVPS
RFSGSGSGTDFTLTISSLQPEDFATYYCQQANSFPWTFGQGTKVEIK
Vopratelimab VH (SEQ ID NO: 112)
EVQLVESGGGLVQPGGSLRLSCAASGFTFSDYWMDWVRQAPGKGLVWVSNIDEDGSIT
EYSPFVKGRFTISRDNAKNTLYLQMNSLRAEDTAVYYCTRWGRFGFDSWGQGTLVTVS
S
Vopratelimab VL (SEQ ID NO: 113)
DIVMTQSPDSLAVSLGERATINCKSSQSLLSGSFNYLTWYQQKPGQPPKLLIFYASTRHT
GVPDRFSGSGSGTDFTLTISSLQAEDVAVYYCHHHYNAPPTFGPGTKVDIK
ICOSL N-terminal fragment (used in acazicolcept)(SEQ ID NO: 114)
DTQEKEVRAMVGSDVELSCACPEGSRFDLNDVYVYWQTSESKTVVTYHIPQHSSLEYV
DSRYRNRALMSPAGMLRGDFSLRLFNVTPQDEQKFHCLVLSRSLGFQEVLSVEVTLHV
AANFSV
acazicolcept (ICOSL fragment-null huFc fusion protein)(SEQ ID NO: 115)
DTQEKEVRAMVGSDVELSCACPEGSRFDLNDVYVYWQTSESKTVVTYHIPQHSSLEYV
DSRYRNRALMSPAGMLRGDFSLRLFNVTPQDEQKFHCLVLSRSLGFQEVLSVEVTLHV
AANFSVGGGGSGGGGSEPKSSDKTHTCPPCPAPEAEGAPSVFLFPPKPKDTLMISRTPEV
TCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALH
NHYTQKSLSLSPG
ICOSL Gly-Ser linker (SEQ ID NO: 116)
GGGGSGGGGS
Lulizumab pegol (pegylated anti-CD28 V-kappa domain antibody (dAb))(SEQ ID NO: 117)
DIQMTQSPSSLSASVGDRVTITCRASRPIWPFLEWYQQKPGKAPKLLIYFTSRLRHGVPSR
FSGSGSGTCFTLTISSLQPEDFATYYCLQNVANPATFSQGTKVEIKR
Abrilumab VH (SEQ ID NO: 118)
QVQLVQSGAEVKKPGASVKVSCKVSGYTLSDLSIHWVRQAPGKGLEWMGGFDPQDGE
TIYAQKFQGRVTMTEDTSTDTAYMELSSLKSEDTAVYYCATGSSSSWFDPWGQGTLVT
VSS
Abrilumab VL (SEQ ID NO: 119)
DIQMTQSPSSVSASVGDRVTITCRASQGISSWLAWYQQKPGKAPKLLIYGASNLESGVPS
RFSGSGSGTDFTLTISSLQPEDFANYYCQQANSFPWTFGQGTKVEIK
Natalizumab VH (SEQ ID NO: 120)
QVQLVQSGAEVKKPGASVKVSCKASGFNIKDTYIHWVRQAPGQRLEWMGRIDPANGY
TKYDPKFQGRVTITADTSASTAYMELSSLRSEDEAVYYCAREGYYGNYGVYAMDYWG
QGTLVTVSS
Natalizumab VL (SEQ ID NO: 121)
DIQMTQSPSSLSASVGDRVTITCKTSQDINKYMAWYQQTPGKAPRLLIHYTSALQPGIPS
RFSGSGSGRDYTFTISSLQPEDIATYYCLQYDNLWTFGQGTKVEIK
Natalizumab Heavy Chain (SEQ ID NO: 122)
QVQLVQSGAEVKKPGASVKVSCKASGFNIKDTYIHWVRQAPGQRLEWMGRIDPANGY
TKYDPKFQGRVTITADTSASTAYMELSSLRSEDEAVYYCAREGYYGNYGVYAMDYWG
QGTLVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVE
Natalizumab Light Chain (SEQ ID NO: 123)
DIQMTQSPSSLSASVGDRVTITCKTSQDINKYMAWYQQTPGKAPRLLIHYTSALQPGIPS
RFSGSGSGRDYTFTISSLQPEDIATYYCLQYDNLWTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNR
Ontamalimab VH (SEQ ID NO: 124)
QVQLVQSGAEVKKPGASVKVSCKASGYTFTSYGINWVRQAPGQGLEWMGWISVYSGN
TNYAQKVQGRVTMTADTSTSTAYMDLRSLRSDDTAVYYCAREGSSSSGDYYYGMDV
WGQGTTVTVSS
Ontamalimab VL (SEQ ID NO: 125)
DIVMTQTPLSLSVTPGQPASISCKSSQSLLHTDGTTYLYWYLQKPGQPPQLLIYEVSNRFS
GVPDRFSGSGSGTDFTLKISRVEAEDVGIYYCMQNIQLPWTFGQGTKVEIK
Ontamalimab Heavy Chain (SEQ ID NO: 126)
QVQLVQSGAEVKKPGASVKVSCKASGYTFTSYGINWVRQAPGQGLEWMGWISVYSGN
TNYAQKVQGRVTMTADTSTSTAYMDLRSLRSDDTAVYYCAREGSSSSGDYYYGMDV
WGQGTTVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK
Ontamalimab Light Chain (SEQ ID NO: 127)
DIVMTQTPLSLSVTPGQPASISCKSSQSLLHTDGTTYLYWYLQKPGQPPQLLIYEVSNRFS
GVPDRFSGSGSGTDFTLKISRVEAEDVGIYYCMQNIQLPWTFGQGTKVEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
adalimumab HCDR1 (SEQ ID NO: 128)
DYAMH
adalimumab HCDR2 (SEQ ID NO: 129)
AITWNSGHIDYADSVEG
adalimumab HCDR3 (SEQ ID NO: 130)
VSYLSTASSLDY
adalimumab LCDR1 (SEQ ID NO: 131)
RASQGIRNYLA
adalimumab LCDR2 (SEQ ID NO: 132)
AASTLQS
adalimumab LCDR3 (SEQ ID NO: 133)
QRYNRAPYT
adalimumab VH (SEQ ID NO: 134)
EVQLVESGGGLVQPGRSLRLSCAASGFTFDDYAMHWVRQAPGKGLEWVSAITWNSGHI
DYADSVEGRFTISRDNAKNSLYLQMNSLRAEDTAVYYCAKVSYLSTASSLDYWGQGTL
VTVSS
adalimumab VL (SEQ ID NO: 135)
DIQMTQSPSSLSASVGDRVTITCRASQGIRNYLAWYQQKPGKAPKLLIYAASTLQSGVPS
RFSGSGSGTDFTLTISSLQPEDVATYYCQRYNRAPYTFGQGTKVEIK
Etanercept TNFR-CYS Repeat 1 (SEQ ID NO: 136)
LPAQVAFTPYAPEPGSTCRLREYYDQTAQMCCSKCSPGQHAKVFCTKTSDTVCD
Etanercept TNFR-CYS Repeat 2 (SEQ ID NO: 137)
SCEDSTYTQLWNWVPECLSCGSRCSSDQVETQACTREQNRIC
Etanercept TNFR-CYS Repeat 3 (SEQ ID NO: 138)
TCRPGWYCALSKQEGCRLCAPLRKCRPGFGVARPGTETSDVVCK
Etanercept TNFR-CYS Repeat 4 (SEQ ID NO: 139)
PCAPGTFSNTTSSTDICRPHQICNVVAIPGNASMDAVCT
Etanercept TNFR-CYS Repeat 5 (SEQ ID NO: 140)
STSPTRSMAPGAVHLPQPVSTRSQHTQPTPEPSTAPSTSFLLPMGPSPPAEGSTGD
Etanercept fusion protein (SEQ ID NO: 141)
LPAQVAFTPYAPEPGSTCRLREYYDQTAQMCCSKCSPGQHAKVFCTKTSDTVCDSCEDS
TYTQLWNWVPECLSCGSRCSSDQVETQACTREQNRICTCRPGWYCALSKQEGCRLCAP
LRKCRPGFGVARPGTETSDVVCKPCAPGTFSNTTSSTDICRPHQICNVVAIPGNASMDAV
CTSTSPTRSMAPGAVHLPQPVSTRSQHTQPTPEPSTAPSTSFLLPMGPSPPAEGSTGDEPK
SCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
golimumab HCDR1 (SEQ ID NO: 142)
SYAMH
golimumab HCDR2 (SEQ ID NO: 143)
FMSYDGSNKKYADSVKG
golimumab HCDR3 (SEQ ID NO: 144)
DRGIAAGGNYYYYGMDV
golimumab LCDR1 (SEQ ID NO: 145)
RASQSVYSYLA
golimumab LCDR2 (SEQ ID NO: 146)
DASNRAT
golimumab LCDR3 (SEQ ID NO: 147)
QQRSNWPPFT
golimumab VH (SEQ ID NO: 148)
QVQLVESGGGVVQPGRSLRLSCAASGFIFSSYAMHWVRQAPGNGLEWVAFMSYDGSN
KKYADSVKGRFTISRDNSKNTLYLQMNSLRAEDTAVYYCARDRGIAAGGNYYYYGMD
VWGQGTTVTVSS
golimumab VL (SEQ ID NO: 149)
EIVLTQSPATLSLSPGERATLSCRASQSVYSYLAWYQQKPGQAPRLLIYDASNRATGIPA
RFSGSGSGTDFTLTISSLEPEDFAVYYCQQRSNWPPFTFGPGTKVDIKRTV
golimumab Heavy Chain (SEQ ID NO: 150)
QVQLVESGGGVVQPGRSLRLSCAASGFIFSSYAMHWVRQAPGNGLEWVAFMSYDGSN
KKYADSVKGRFTISRDNSKNTLYLQMNSLRAEDTAVYYCARDRGIAAGGNYYYYGMD
VWGQGTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
golimumab Light Chain Heavy Chain (SEQ ID NO: 151)
EIVLTQSPATLSLSPGERATLSCRASQSVYSYLAWYQQKPGQAPRLLIYDASNRATGIPA
RFSGSGSGTDFTLTISSLEPEDFAVYYCQQRSNWPPFTFGPGTKVDIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
infliximab VH Heavy Chain (SEQ ID NO: 152)
EVKLEESGGGLVQPGGSMKLSCVASGFIFSNHWMNWVRQSPEKGLEWVAEIRSKSINS
ATHYAESVKGRFTISRDDSKSAVYLQMTDLRTEDTGVYYCSRNYYGSTYDYWGQGTTL
TVS
infliximab VL Heavy Chain (SEQ ID NO: 153)
DILLTQSPAILSVSPGERVSFSCRASQFVGSSIHWYQQRINGSPRLLIKYASESMSGIPSRF
SGSGSGTDFTLSINTVESEDIADYYCQQSHSWPFTFGSGTNLEVK
infliximab HCDR1 Heavy Chain (SEQ ID NO: 154)
NHWMN
infliximab HCDR2 Heavy Chain (SEQ ID NO: 155)
EIRSKSINSATHYAESVKG
infliximab HCDR3 Heavy Chain (SEQ ID NO: 156)
NYYGSTYDY
infliximab LCDR1 Heavy Chain (SEQ ID NO: 157)
RASQFVGSSIH
infliximab LCDR2 Heavy Chain (SEQ ID NO: 158)
YASESMS
infliximab LCDR3 Heavy Chain (SEQ ID NO: 159)
QQSHSWPFT
certolizumab pego1 HCDR1 Heavy Chain (SEQ ID NO: 160)
DYGMN
certolizumab pego1 HCDR2 (SEQ ID NO: 161)
WINTYIGEPIYADSVKG
certolizumab pego1 HCDR3 (SEQ ID NO: 162)
GYRSYAMDY
certolizumab pego1 LCDR1 (SEQ ID NO: 163)
NVGTNVA
certolizumab pego1 LCDR2 (SEQ ID NO: 164)
SASFLYS
certolizumab pego1 LCDR3 (SEQ ID NO: 165)
QQYNIYPLTF
certolizumab pego1 VH (SEQ ID NO: 166)
EVQLVESGGGLVQPGGSLRLSCAASGYVFTDYGMNWVRQAPGKGLEWMGWINTYIGE
PIYADSVKGRFTFSLDTSKSTAYLQMNSLRAEDTAVYYCARGYRSYAMDYWGQGTLV
TVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCAA
certolizumab pego1 VL (SEQ ID NO: 167)
DIQMTQSPSSLSASVGDRVTITCKASQNVGTNVAWYQQKPGKAPKALIYSASFLYSGVP
YRFSGSGSGTDFTLTISSLQPEDFATYYCQQYNIYPLTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
certolizumab pego1 VH (SEQ ID NO: 168)
EVQLVESGGGLVQPGGSLRLSCAASGYVFTDYGMNWVRQAPGKGLEWMGWINTYIGE
PIYADSVKGRFTFSLDTSKSTAYLQMNSLRAEDTAVYYCARGYRSYAMDYWGQGTLV
TVSS
Sibeprenlimab VH (SEQ ID NO: 169)
QVQLVQSGAEVKKPGASVKVSCKASGYTFTDYTIHWVRQATGQGLEWMGWIYPLRGS
INYAQKFQGRVTMTADKSISTVYMELSSLRSEDTAVYFCARHGAYYSNAFDYWGQGTL
VTVSS
Sibeprenlimab VL (SEQ ID NO: 170)
EIVMTQSPATLSVSPGERATLSCRASESVDNDGIRFLHWYQQKPGQAPRLLIYRASTRAT
GIPARFSGSGSRTEFTLTISSLQSEDFAVYYCQQSNKDPYTFGGGTKVEIK
Sibeprenlimab Heavy Chain (SEQ ID NO: 171)
QVQLVQSGAEVKKPGASVKVSCKASGYTFTDYTIHWVRQATGQGLEWMGWIYPLRGS
INYAQKFQGRVTMTADKSISTVYMELSSLRSEDTAVYFCARHGAYYSNAFDYWGQGTL
VTVSSASTKGPSVFPLAPCSRSTSESTAALGCLVKDYFPEPVTVSWNSGALTSGVHTFPA
VLQSSGLYSLSSVVTVPSSNFGTQTYTCNVDHKPSNTKVDKTVERKCCVECPPCPAPPV
AGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPP
SREEMTKNQVSLTCLVKGFYPSDIAVEWESNGQPENNYKTTPPMLDSDGSFFLYSKLTV
DKSRWQQGNVFSCSVMHEALHNHYTQKSLSLSPGK
Sibeprenlimab Light Chain (SEQ ID NO: 172)
EIVMTQSPATLSVSPGERATLSCRASESVDNDGIRFLHWYQQKPGQAPRLLIYRASTRAT
GIPARFSGSGSRTEFTLTISSLQSEDFAVYYCQQSNKDPYTFGGGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
BION-1301 HCDR1 (SEQ ID NO: 173)
GYTFTSYV
BION-1301 HCDR2 (SEQ ID NO: 174)
YINPYNDAPKYNEKFK
BION-1301 HCDR3 (SEQ ID NO: 175)
GLGYALYYAMDY
BION-1301 LCDR1 (SEQ ID NO: 176)
KASQNVGNNVA
BION-1301 LCDR2 (SEQ ID NO: 177)
SASNRDS
BION-1301 LCDR3 (SEQ ID NO: 178)
QQYNIYPFTF
BION-1301 VH (SEQ ID NO: 179)
QVQLVQSGAEVKKPGASVKVSCKASGYTFTSYVMHWVRQAPGQGLEWMGYINPYND
APKYNEKFKGRVTMTRDTSISTAYMELSRLRSDDTAVYYCARGLGYALYYAMDYWGQ
GTTVTVSS
BION-1301 VL (SEQ ID NO: 180)
DIVMTQSPSTLSASVGDRVTITCKASQNVGNNVAWYQQKPGKAPKLLISSASNRDSGVP
SRFSGSGSGTEFTLTISSLQPDDFATYFCQQYNIYPFTFGQGTKLEIK
human IgG1 constant region (SEQ ID NO: 181)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
human IgG1 null constant region (SEQ ID NO: 182)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAG
APSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPS
REEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
human kappa constant region (SEQ ID NO: 183)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
bleselumab variant 1 heavy chain (SEQ ID NO: 184)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAG
APSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPS
REEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
bleselumab variant 1 heavy chain variable region (SEQ ID NO: 185)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
S
bleselumab variant 1 heavy chain constant domain (SEQ ID NO: 186)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAG
APSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPS
REEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
bleselumab variant 2 heavy chain (SEQ ID NO: 187)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAEG
APSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPSSIEKTISKAKGQPREPQVYTLPPS
REEMTKNQVSLTCLVKGFYPSDIA VEWESNGQPENNYKTTPPVLDSDGSFFLYSKLTVD
KSRWQQGNVFSCSVMHEALHNHYTQKSLSLSPGK
bleselumab variant 2 heavy chain variable region (SEQ ID NO: 188)
QLQLQESGPGLLKPSETLSLTCTVSGGSISSPGYYGGWIRQPPGKGLEWIGSIYKSGSTYH
NPSLKSRVTISVDTSKNQFSLKLSSVTAADTAVYYCTRPVVRYFGWFDPWGQGTLVTVS
S
bleselumab variant 2 heavy chain constant domain (SEQ ID NO: 189)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAEGA
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPSSIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
bleselumab variant 1 light chain (SEQ ID NO: 190)
AIQLTQSPSSLSASVGDRVTITCRASQGISSALAWYQQKPGKAPKLLIYDASNLESGVPSR
FSGSGSGTDFTLTISSLQPEDFATYYCQQFNSYPTFGQGTKVEIKRTVAAPSVFIFPPSDEQ
LKSGTASVVCLLNNFYPREAKVQWKVDNALQSGNSQESVTEQDSKDSTYSLSSTLTLSK
ADYEKHKVYACEVTHQGLSSPVTKSFNRGEC
bleselumab variant 1 light chain variable region (SEQ ID NO: 191)
AIQLTQSPSSLSASVGDRVTITCRASQGISSALAWYQQKPGKAPKLLIYDASNLESGVPSR
FSGSGSGTDFTLTISSLQPEDFATYYCQQFNSYPTFGQGTKVEIK
bleselumab variant 1 light chain constant domain (SEQ ID NO: 192)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
variant 3 heavy chain (SEQ ID NO: 193)
QVQLQESGPGLVKPSETLSLTCTVSGGSIRGYYWSWIRQPPGKGLEWIGYIYYSGSTNYN
PSLKSRVTISVDTSKNQFSLKLSSVTAADTAVYYCARRGGLYGDYGWFAPWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEA
AGAPSVFLFPPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
variant 3 heavy chain variable region (SEQ ID NO: 194)
QVQLQESGPGLVKPSETLSLTCTVSGGSIRGYYWSWIRQPPGKGLEWIGYIYYSGSTNYN
PSLKSRVTISVDTSKNQFSLKLSSVTAADTAVYYCARRGGLYGDYGWFAPWGQGTLVT
VSS
variant 3 heavy chain constant domain (SEQ ID NO: 195)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAAG
APSVFLFPPKPKDTLMISRTPEVTCVVVDVSHEDPEVKFNWYVDGVEVHNAKTKPREEQ
YNSTYRVVSVLTVLHQDWLNGKEYKCAVSNKALPAPIEKTISKAKGQPREPQVYTLPPS
REEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
variant 4 heavy chain (SEQ ID NO: 196)
QVQLQESGPGLVKPSETLSLTCTVSGGSIRGYYWSWIRQPPGKGLEWIGYIYYSGSTNYN
PSLKSRVTISVDTSKNQFSLKLSSVTAADTAVYYCARRGGLYGDYGWFAPWGQGTLVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEA
EGAPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPSSIEKTISKAKGQPREPQVYTLP
PSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
variant 4 heavy chain variable region (SEQ ID NO: 197)
QVQLQESGPGLVKPSETLSLTCTVSGGSIRGYYWSWIRQPPGKGLEWIGYIYYSGSTNYN
PSLKSRVTISVDTSKNQFSLKLSSVTAADTAVYYCARRGGLYGDYGWFAPWGQGTLVT
VSS
variant 4 heavy chain constant domain (SEQ ID NO: 198)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPEAEGA
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPSSIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
variant 2 light chain (SEQ ID NO: 199)
EIVLTQSPGTLSLSPGERATLSCRASQSVSSTYLAWYQQKPGQAPRLLIYGASSRATGIPD
RFSGSGSGTDFTLTISRLEPEDFAVYYCQQYSSLFTFGPGTKVDIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
variant 2 light chain variable region (SEQ ID NO: 200)
EIVLTQSPGTLSLSPGERATLSCRASQSVSSTYLAWYQQKPGQAPRLLIYGASSRATGIPD
RFSGSGSGTDFTLTISRLEPEDFAVYYCQQYSSLFTFGPGTKVDIK
variant 2 light chain constant domain (SEQ ID NO: 201)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1-11. (canceled)

12. A compound of Formula I: 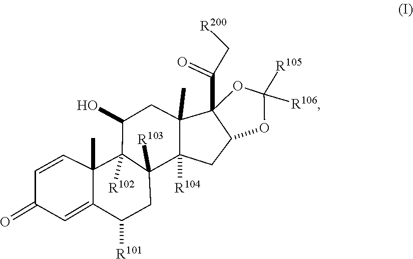
or a pharmaceutically acceptable salt thereof, wherein:
R¹⁰¹, R¹⁰², R¹⁰³, and R¹⁰⁴ are each independently H or F;
R¹⁰⁵ is C₂₋₆ alkenyl or heteroaryl wherein the alkenyl is substituted with 1, 2, or 3 R¹⁰⁷ and the heteroaryl is substituted with 1, 2 or 3 R¹¹⁰;
R¹⁰⁶ is H;
each R¹⁰⁷ is independently C₂₋₆ alkoxyalkyl, C₁₋₆ haloalkoxy, phenyl, heteroaryl, C₃₋₈ cycloalkyl, heterocyclyl, or halogen, wherein the phenyl is substituted with 1, 2, or 3 R¹¹¹, and the heteroaryl, cycloalkyl, or heterocyclyl is substituted with 0, 1, 2, or 3 R¹¹¹;
each R¹¹⁰ is independently C₂₋₆ alkyl, C₂₋₆ alkenyl, C₂₋₆ alkynyl, C₂₋₆ alkoxyalkyl, —(C₁₋₆ alkylene)-OR¹¹⁴, —(C₁₋₆ alkylene)-N(R¹¹⁴)₂, C₁₋₆ haloalkyl, phenyl, —(C₁₋₆ alkylene)-phenyl, heteroaryl, —(C₁₋₆ alkylene)-heteroaryl, halogen, —N₃, —OR¹¹⁵, —N(R¹¹⁵)₂, —N(R¹¹⁵)(CO)R¹¹⁵, —N(R¹¹⁵)(CO)OR¹¹⁵, —N(R¹¹⁵)S(O)₂R¹¹⁵, —(CO)R¹¹⁵, —SO₂R¹¹⁵, or —SO₂N(R¹¹⁵)₂, wherein the phenyl, alkylene-phenyl, heteroaryl, or alkylene-heteroaryl is substituted with 0, 1, 2, or 3 R¹¹⁶;
each R¹¹¹ and R¹¹² is independently C₁₋₆ alkyl, C₁₋₆ alkoxy, C₁₋₆ haloalkyl, C₁₋₆ haloalkoxy, halogen, —OR¹¹⁴ or —N(R¹¹⁴)²;
each R¹¹³ is independently H, C₁₋₆ alkyl, C₁₋₆ alkoxy, C₁₋₆ haloalkyl, or phenyl, wherein the haloalkyl is substituted with 0 or 1 N(R¹¹⁴)₂;
each R¹¹⁴ is independently H or C₁₋₆ alkyl;
each R¹¹⁶ is —OR¹¹⁷, —N(R¹¹⁷)₂, —N(R¹¹⁷)(CO)R¹¹⁷, —N(R¹¹⁷)(CO)OR¹¹⁷, —N(R¹¹⁷)S(O)₂R¹¹⁷, —(CO)R¹¹⁷, —SO₂R¹¹⁷, —SO₂N(R¹¹⁷)₂, or R³⁰⁰;
each R¹¹⁵ and R¹¹⁷ is independently H, C₁₋₆ alkyl, C₁₋₆ haloalkyl, phenyl, or R³⁰⁰;
R²⁰⁰ is —OR²⁰¹ or —N(R²⁰¹)₂;
R²⁰¹ is H, C₁₋₆ alkyl, phenyl, or heteroaryl, wherein the phenyl or heteroaryl is substituted with 0, 1, or 2 —OR²⁰², —N(R²⁰²)₂ or R³⁰⁰;
R²⁰² is H or C₁₋₆ alkyl; and
R³⁰⁰ has one of the following structures: 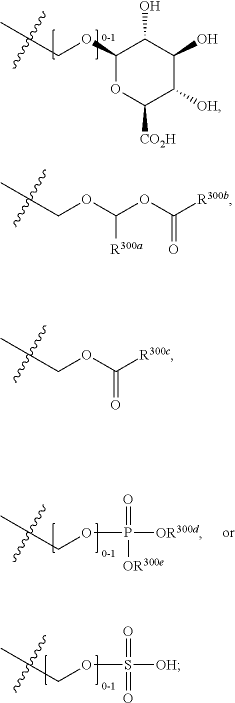
wherein:
R^{300a} is H or C₁₋₆ alkyl;
R^{300b} is C₁₋₆ alkyl or C₁₋₆ alkoxy;
R^{300c} is H, C₁₋₆ alkyl, —CH₂OH, or C₁₋₆ alkoxy;
R^{300d} is H or C₁₋₆ alkyl; and
R^{300e} is H or C₁₋₆ alkyl;
wherein the heteroaryl in each instance is a 5- to 10-membered heteroaryl having 1, 2, or 3 heteroatoms selected from N, O, and S; and
the heterocyclyl in each instance is a 4- to 10-membered heterocyclyl having 1, 2, or 3 heteroatoms selected from N, O, and S.

13-19. (canceled)

20. The compound of claim 12, or a pharmaceutucally acceptable salt thereof, wherein R¹⁰⁵ is 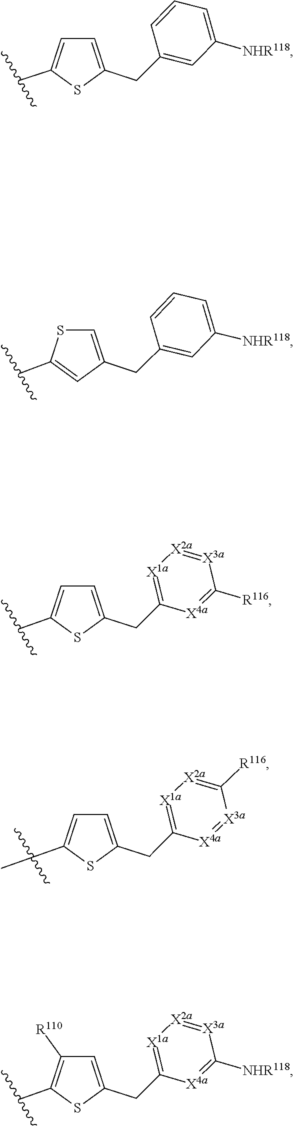 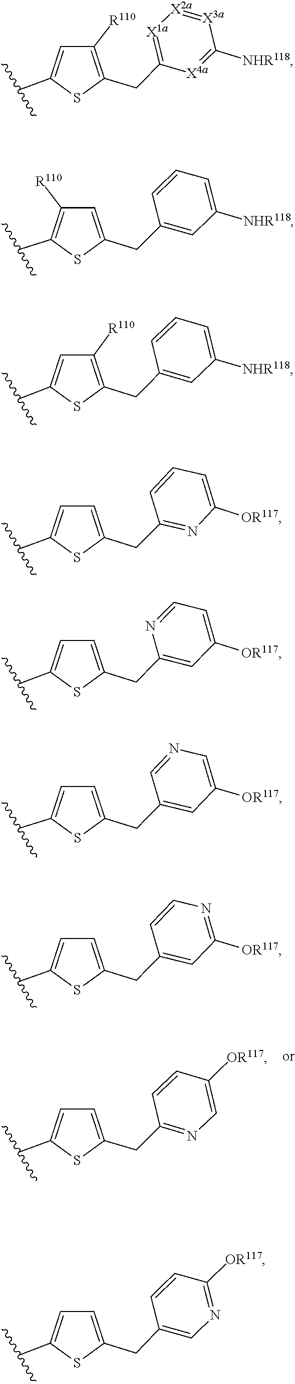
wherein
each X^{1a}, X^{2a}, X^{3a}, and X^{4a} is independently CH or N;
R¹¹⁰ is CH₃, CH₂F, CHF₂, or CF₃;
R¹¹⁶ is —NH(CO)CH₃, —NHS(O)₂CH₃ or R³⁰⁰; and
R¹¹⁷ is CH₃, CH₂F, CHF₂, CF₃, or R³⁰⁰:
R¹¹⁸ is H or R³⁰⁰.

21. (canceled)

22. The compound of claim 12, wherein R¹⁰³, and R¹⁰⁴ are each H.

23. The compound of claim 12, wherein R¹⁰¹ and R¹⁰² are each H.

24. The compound of claim 12, wherein R¹⁰¹ and R¹⁰² are each F.

25. The compound of claim 12, wherein R¹⁰¹ is F, and R¹⁰² is H.

26. The compound of claim 12, wherein R¹⁰¹ is F, and R¹⁰² is H.

27. (canceled)

28. A conjugate comprising:
(a) a compound of claim 12, or a pharmaceutically acceptable salt thereof;
(b) a binding protein comprising a binding domain capable of specifically binding to a target or multiple targets, wherein the target is selected from the group consisting of CD40,
CD40 Ligand, T-lymphocyte activation antigen CD86 (CD86), cytotoxic T-lymphocyte protein 4 (CTLA4), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), tumor necrosis factor receptor 2 (TNF-R2), killer cell lectin-like receptor G1 (KLRG1), B-cell-activating factor (BAFF), BAFF Receptor (BAFFR), transmembrane activator and CAML interactor (TACI), Peyer patches-specific homing receptor (LPAM-1), B-cell maturation antigen (BCMA), and a proliferation-inducing ligand (APRIL); and
(c) a linker covalently attaching the compound to the binding protein.

29-44. (canceled)

45. A pharmaceutical composition comprising a compound of claim 12, or a pharmaceutically acceptable salt thereof and a pharmaceutically acceptable excipient.

46. (canceled)

47. A method of treating or preventing an autoimmune or inflammatory condition in a subject in need thereof, comprising administering to the subject an effective amount of the pharmaceutical composition of claim 45.

48-49. (canceled)

50. The compound of claim 12, wherein R¹⁰⁵ has one of the following structures:

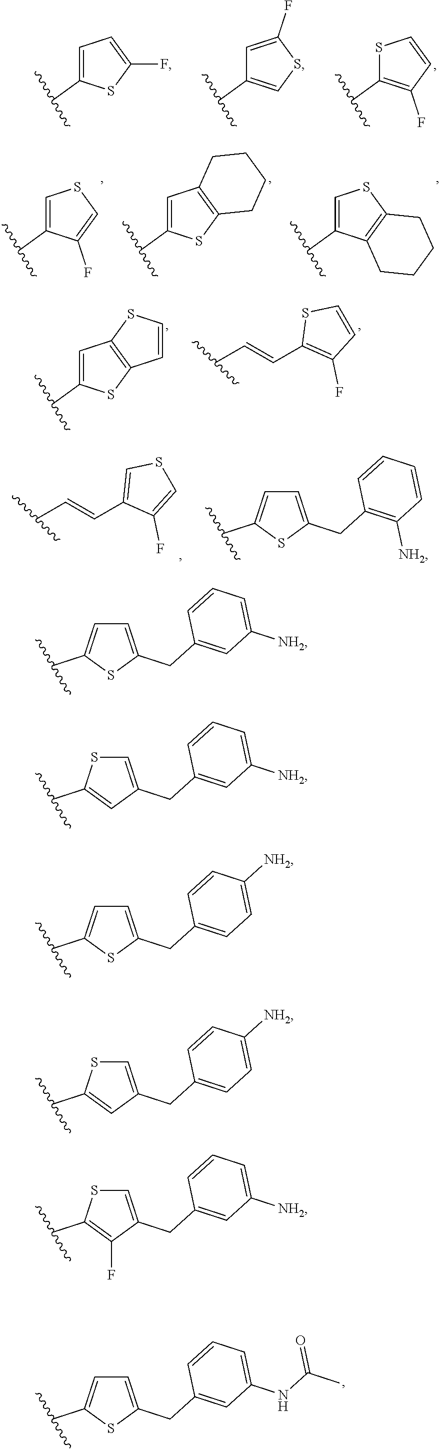

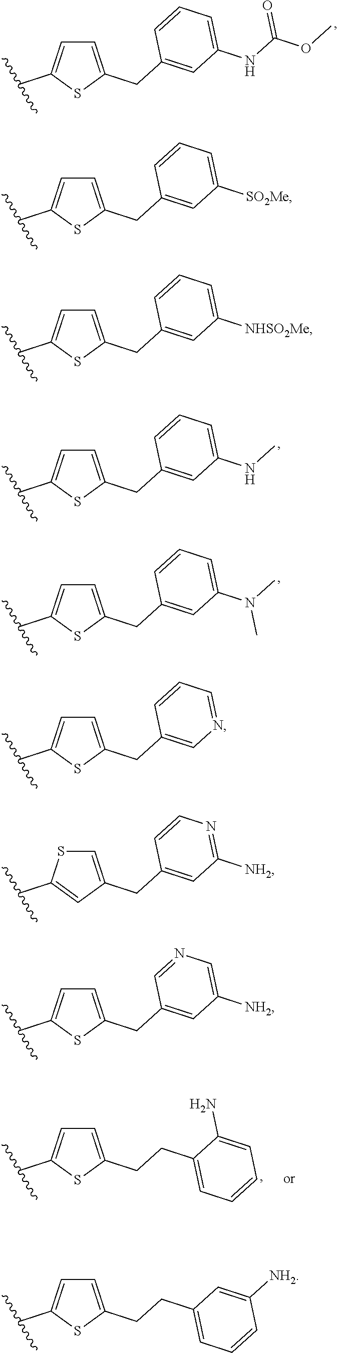

51. A compound having the following structure: 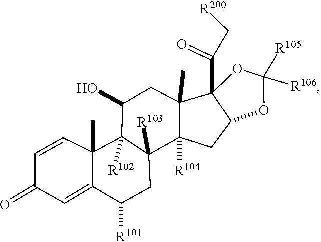
or a pharmaceutically acceptable salt thereof, wherein
R¹⁰¹, R¹⁰², R¹⁰³, and R¹⁰⁴ are each independently H or F;
R¹⁰⁵ has one of the following structures: 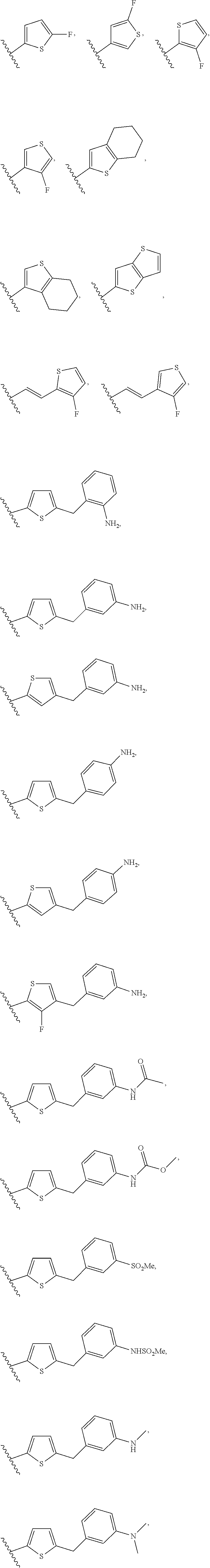 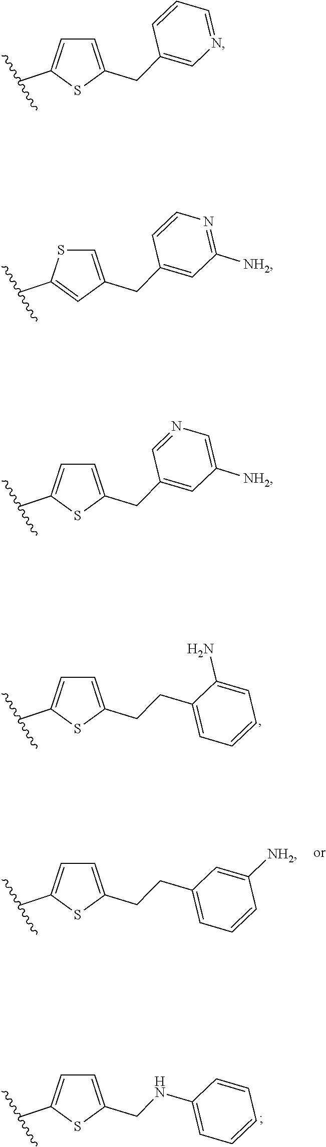
R¹⁰⁶ is H;
R²⁰⁰ is —OR²⁰¹ or —N(R²⁰¹)₂;
R²⁰¹ is H, C₁₋₆ alkyl, phenyl, or heteroaryl, wherein the phenyl or heteroaryl is substituted with 0, 1, or 2 —OR²⁰², —N(R²⁰²)₂ or R³⁰⁰;
R²⁰² is H or C₁₋₆ alkyl; and
R³⁰⁰ has one of the following structures: 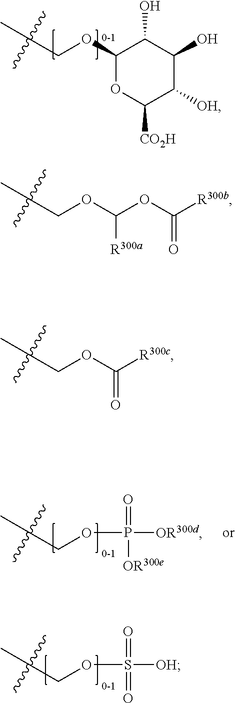
wherein:
R^{300a} is H or C₁₋₆ alkyl;
R^{300b} is C₁₋₆ alkyl or C₁₋₆ alkoxy;
R^{300c} is H, C₁₋₆ alkyl, —CH₂OH, or C₁₋₆ alkoxy;
R^{300d} is H or C₁₋₆ alkyl; and
R^{300e} is H or C₁₋₆ alkyl;
wherein the heteroaryl in each instance is a 5- to 10-membered heteroaryl having 1, 2, or 3 heteroatoms selected from N, O, and S.

52. A compound having one of the following structures:

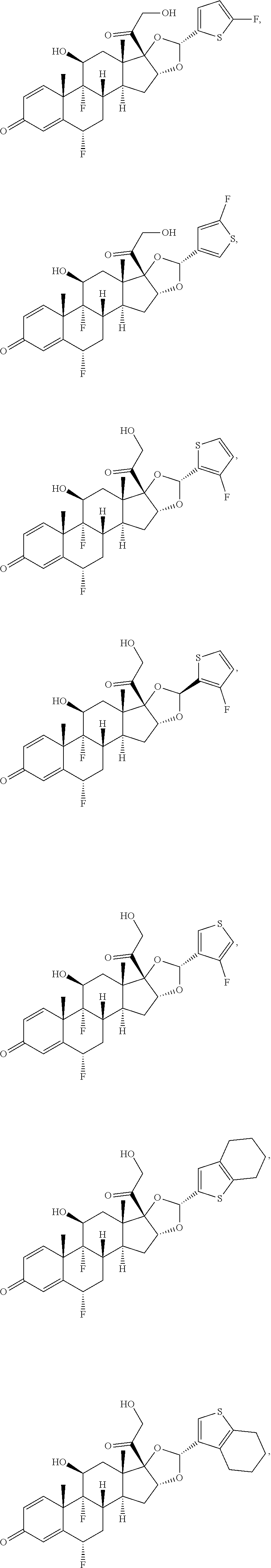

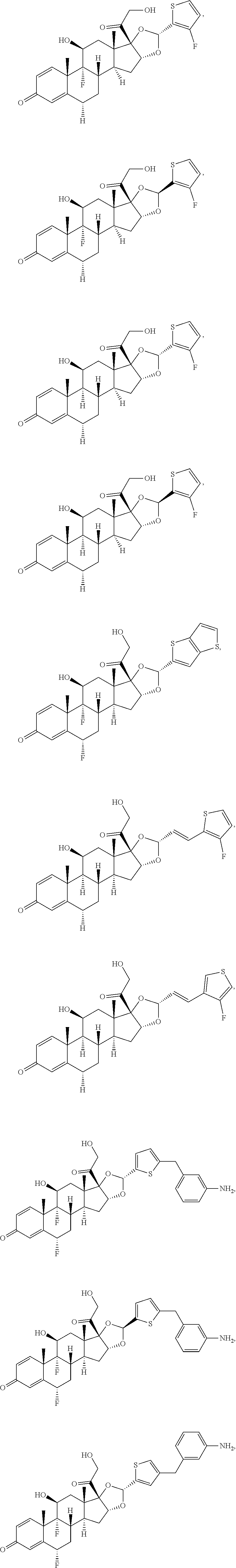

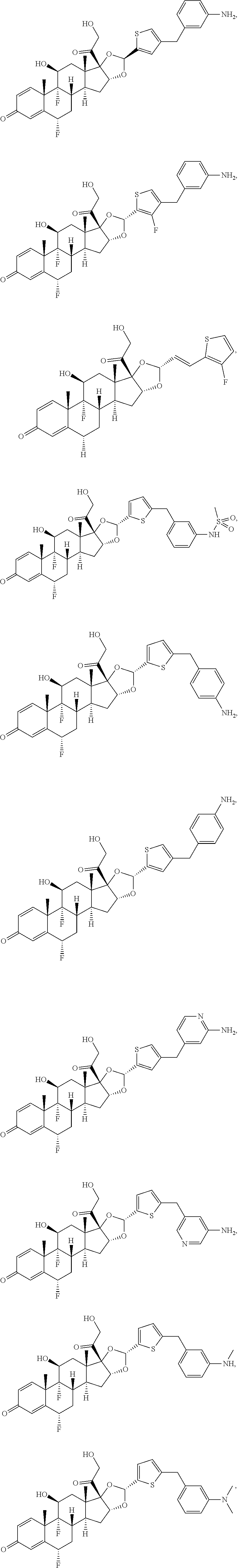

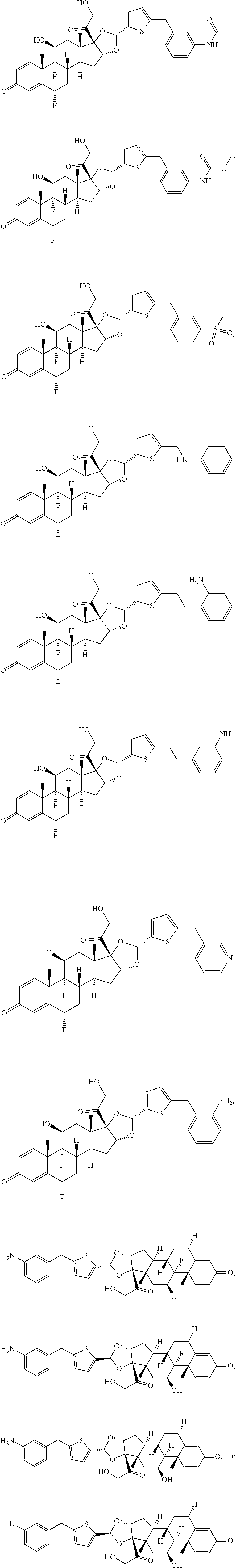

53. A conjugate comprising:
(a) a compound of claim 51, or a pharmaceutically acceptable salt thereof,
(b) a binding protein comprising a binding domain capable of specifically binding to a target or multiple targets, wherein the target is selected from the group consisting of CD40,
CD40 Ligand, T-lymphocyte activation antigen CD86 (CD86), cytotoxic T-lymphocyte protein 4 (CTLA4), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), tumor necrosis factor receptor 2 (TNF-R2), killer cell lectin-like receptor G (KLRG1), B-cell-activating factor (BAFF), BAFF Receptor (BAFFR), transmembrane activator and CAML interactor (TALI), Peyer patches-specific homing receptor (LPAM-1), B-cell maturation antigen (BCMA), and a proliferation-inducing ligand (APRIL); and
(c) a linker covalently attaching the compound to the binding protein.

54. A pharmaceutical composition comprising the compound of claim 51 or a pharmaceutically acceptable salt thereof and a pharmaceutically acceptable excipient.

55. A method of treating or preventing an auto immune or inflammatory condition in a subject in need thereof, comprising administering to the subject an effective amount of the pharmaceutical composition of claim 54.

56. A conjugate comprising:
(a) a compound of claim 52, or a pharmaceutically acceptable salt thereof,
(b) a binding protein comprising a binding domain capable of specifically binding to a target or multiple targets, wherein the target is selected from the group consisting of CD40, CD40 Ligand, T-lymphocyte activation antigen CD86 (CD86), cytotoxic T-lymphocyte protein 4 (CTLA4), inducible T-cell costimulator (ICOS), ICOS Ligand (ICOSL), T-cell-specific surface glycoprotein CD28 (CD28), T-lymphocyte activation antigen CD80 (CD80), integrin β7, Integrin α4, mucosal addressin cell adhesion molecule 1 (MADCAM), tumor necrosis factor alpha (TNFα), tumor necrosis factor receptor 2 (TNF-R2), killer cell lectin-like receptor G1 (KLRG1), B-cell-activating factor (BAFF), BAFF Receptor (BAFFR), transmembrane activator and CAML interactor (TALI), Peyer patches-specific homing receptor (LPAM-1), B-cell maturation antigen (BCMA), and a proliferation-inducing ligand (APRIL); and
(c) a linker covalently attaching the compound to the binding protein.

57. A pharmaceutical composition comprising the compound of claim 52 or a pharmaceutically acceptable salt thereof and a pharmaceutically acceptable excipient.

58. A method of treating or preventing an auto immune or inflammatory condition in a subject in need thereof, comprising administering to the subject an effective amount of the pharmaceutical composition of claim 57.

59. The compound of claim 51, wherein R²⁰⁰ is —OH.

60. A linker-payload molecule comprising:
(a) a compound of claim 12; and
(b) a linker covalently attached to the compound and covalently attached to a functional group capable of covalently linking the linker to a binding protein.

61. A linker-payload molecule comprising:
(a) a compound of claim 51; and
(b) a linker covalently attached to the compound and covalently attached to a functional group capable of covalently linking the linker to a binding protein.

62. A linker-payload molecule comprising:
(a) a compound of claim 52; and
(b) a linker covalently attached to the compound and covalently attached to a functional group capable of covalently linking the linker to a binding protein.
